(19) United States
(12) Patent Application Publication
(10) Pub. No.: US 2026/0138998 A1
(43) Pub. Date: May 21, 2026

(54) GLP-1 RECEPTOR AGONISTS AND METHODS OF USE

(71) Applicant: Merck Sharp & Dohme LLC, Rahway, NJ (US)

(72) Inventors: Vlad Bacauanu, San Francisco, CA (US); Zhiyong Hu, Livingston, NJ (US); Salman Jabri, Moraga, CA (US); Rebecca Elizabeth Johnson, Oakland, CA (US); Devleena M. Shivakumar, Menlo Park, CA (US); Emma H. Southgate, Half Moon Bay, CA (US); Leah Stateman, San Francisco, CA (US); Brandon M. Taoka, Pacifica, CA (US); Mycah Uehling, New Brunswick, NJ (US); Ethan Wappes, San Francisco, CA (US); Xin Wen, Belmont, CA (US); Brandon A. Wright, San Francisco, CA (US); Rose Yen, San Francisco, CA (US); Meng Yao Zhang, San Mateo, CA (US)

(73) Assignee: Merck Sharp & Dohme LLC, Rahway, NJ (US)

(21) Appl. No.: 19/369,471

(22) Filed: Oct. 27, 2025

Related U.S. Application Data
(60) Provisional application No. 63/712,726, filed on Oct. 28, 2024.
(60) Provisional application No. 63/873,205, filed on Aug. 29, 2025.

(51) Int. Cl. C07F 9/6561; A61K 31/437; A61K 31/438; A61K 31/439; A61K 31/444; A61K 31/4545; A61K 31/46; A61K 31/4709; A61K 31/4725; A61K 31/501; A61K 31/506; A61K 31/513; A61K 31/5377; A61K 31/5386; A61K 31/55; A61K 31/675; C07B 59/00; C07D 471/04; C07D 471/18; C07D 471/20; C07D 487/04; C07D 491/20; C07D 519/00

(52) U.S. Cl. CPC C07F 9/6561; A61K 31/437; A61K 31/438; A61K 31/439; A61K 31/444; A61K 31/4545; A61K 31/46; A61K 31/4709; A61K 31/4725; A61K 31/501; A61K 31/506; A61K 31/513; A61K 31/5377; A61K 31/5386; A61K 31/55; A61K 31/675; C07B 59/002; C07D 471/04; C07D 471/18; C07D 471/20; C07D 487/04; C07D 491/20; C07D 519/00; C07B 2200/05

(57) ABSTRACT

The present invention relates to GLP-1 receptor agonists compounds of Formula I:

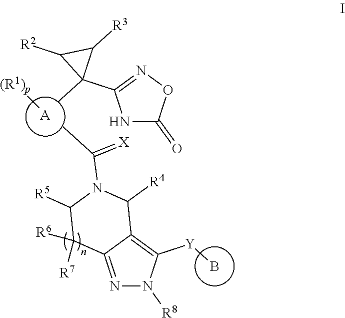

and pharmaceutically acceptable salts thereof, wherein R¹, R², R³, R⁴, R⁵, R⁶, R⁷, R⁸, n, p, A, X, Y and B are as defined herein. The present invention also relates to compositions which comprise a GLP-1 receptor agonist compound of the invention or a pharmaceutically acceptable salt thereof, and a pharmaceutically acceptable carrier. The invention further relates to methods for treating obesity or non-insulin-dependent diabetes mellitus (Type 2 diabetes) comprising administering to a patient a therapeutically effective amount of a compound of the invention.

### CROSS-REFERENCE TO RELATED APPLICATIONS

This application claims priority to and benefit of U.S. Provisional Application Nos. 63/712,726, filed Oct. 28, 2024, and 63/873,205, filed Aug. 29, 2025, the disclosures of which are hereby incorporated by reference in their entireties.

### FIELD

This disclosure relates generally to compounds which are GLP-1 receptor agonists and their uses. A preferred use of GLP-1 receptor agonists is for the treatment of obesity.

### BACKGROUND

Glucagon-like peptide-1 (GLP-1) is a hormone secreted by L cells in the small intestine. GLP-1 consists of 30 or 31 amino acids and acts through the GLP-1 receptor to exert its effects. These effects include promotion of glucose dependent insulin secretion, inhibition of glucagon secretion, delay of gastric emptying, and suppression of feeding. As a result, GLP-1 analogs have been developed as effective therapeutic agents for diabetes and obesity, showing significant benefits in reducing HbA1c levels and promoting weight loss.

There is a need for small molecule GLP-1 receptor agonists for treating various disorders, including obesity.

### SUMMARY

The present invention is directed to compounds which are GLP-1 receptor agonists. The compounds, and their pharmaceutically acceptable salts, are useful, for example, for the treatment of obesity. More particularly, the present invention provides in one embodiment (embodiment no. 1) compounds of Formula I:

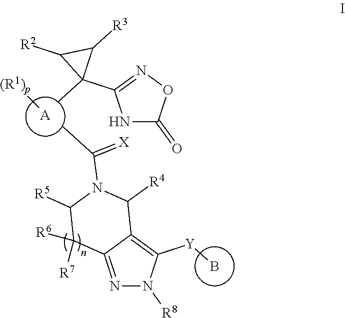

or a pharmaceutically acceptable salt thereof,
wherein:
R¹ is halogen, CN, OH, aminoalkyl, hydroxyalkyl, diaminoalkyl, dihydroxyalkyl, C₁-C₆ alkyl, heteroalkyl, C₁-C₆ heteroalkyl, C₃-C₈ cycloalkyl, 3-10 membered heterocycloalkyl, —C≡C-(3-10 membered heterocycloalkyl), —C≡C—(C₃-C₈ cycloalkyl), or -phenyl-(C₃-C₈ cycloalkyl), wherein R¹ is substituted with 0 to 5 substituents independently selected from halogen, CN, OH, C₁-C₆ alkyl, 3-10 membered heterocycloalkyl or C₃-C₈ cycloalkyl, wherein the C₁-C₆ alkyl, 3-10 membered heterocycloalkyl or C₃-C₈ cycloalkyl is substituted with one or more halogen atoms, C₁-C₂ alkyl, or C₁-C₆ alkyloxy;
R² is H or methyl;
R³ is H or methyl;
R⁴ is hydrogen or C₁-C₆ alkyl;
R⁵ is hydrogen or C₁-C₆ alkyl; or
R⁴ and R⁵, together with the atom which they are attached, or R⁴ and R⁶, together with the atom which they are attached, form a bridged C₇-C₈ heterocyclic ring fused to the pyrazole; each R⁶ is independently H, halo, or C₁-C₆ alkyl and each R⁷ is independently H, halo, or C₁-C₆ alkyl, or
R⁶ and R⁷ together with the atom to which they are attached, form a C₃-C₆ cycloalkyl ring or a 3-6 membered heterocycloalkyl ring, wherein the C₃-C₆ cycloalkyl ring or the 3-6 membered heterocycloalkyl ring is substituted with 0 or 1 instances of R⁹, provided that if there are two R⁶ and R⁷ then only one set forms a ring and the other R⁶ and R⁷ is each independently H or C₁-C₆ alkyl;
R⁹ is H, C₁-C₆ alkyl, —SO₂—(C₁-C₆ alkyl), —((C₁-C₆ alkylenyl)O)ₜ(C₁-C₆ alkyl), —(C₁-C₆ alkylenyl)(C₃-C₈cycloalkyl), —(C)(O)(C₁-C₆ alkyl), —(CH₂)aryl, C₃-C₈ cycloalkyl, —(CH₂)(C₃-C₈ cycloalkyl), C(O)C₁-C₆ alkyl, —((C₁-C₆ alkylenyl)O)ₜ(C₁-C₆ alkyl), or S(O)₂(C₁-C₆ alkyl) wherein one or more of the hydrogen atoms of the C₁-C₆ alkyl may be substituted with fluoro or hydroxyl, and 0 or 1 of the carbons of the C₁-C₆ alkyl form a cyclopropyl ring, and wherein t is 1, 2, 3, 4 or 5;
n is 1 or 2;
p is 1, 2, 3, 4 or 5;
R⁴, R⁵, R⁶ and R⁷ are each independently substituted with 0 to 2 substituents independently selected from halogen, NH₂, aminoalkyl, diaminoalkyl, C₁-C₆ alkyl, and C₁-C₆ alkyl substituted with one or more halogen atoms, C₁-C₆ alkoxy, hydroxy, oxo, CONR^{w1}R^{w2}, SO₂N R^{w3}R^{w4}, SO₂R^{w5}, N(R^{w6})COR^{w7}, NR^{w8}SO₂R^{w9}, wherein R^{w1}, R^{w2}, R^{w3}, R^{w4}, R^{w5}, R^{w6}, R^{w7},R^{w8}, and R^{w9}, are each individually selected from H and C₁-C₆ alkyl;
A is 5-10 membered heteroaryl or C₆-C₁₀ aryl;
X is O, S, or NR^{XX} wherein R^{XX} is H or C₁-C₃ alkyl, wherein C₁-C₃ alkyl is substituted with 0, 1, or 2 substituents selected from halogen and C₃-C₆ cycloalkyl;
Y is

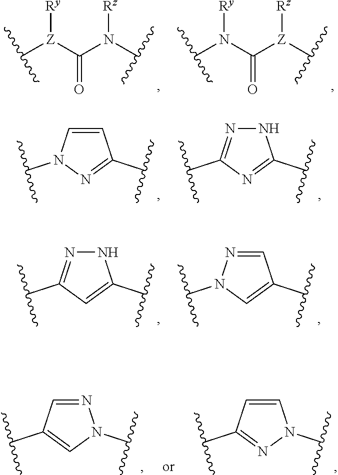

wherein the left [symbol] on Y is bound to the pyrazole moiety and the right [symbol] on Y is bound to B,
Z is C, CR^{x} or N;
R^{x} is H, halo, hydroxy, C₁-C₆ alkyl, C₃-C₆ cycloalkyl, C₁-C₆ alkoxy, or —O(C₃-C₆ cycloalkyl);
R^{y}, and R^{z}, together with the atom which they are attached and optionally R^{X}, form a 5-8 membered heterocycloalkyl ring or a 5-6 membered heteroaryl; or
R^{y}, and R^{z} are each independently H, halo, C₁-C₆ alkyl, or C₃-C₆ cycloalkyl; or
R^{x} and R^{y}, together with the carbon to which they are attached, form a spiro C₃-C₆ cycloalkyl ring and R^{z} is H, halo, or C₁-C₆ alkyl; or
R^{x} and R^{z}, together with the carbon to which they are attached, form a spiro C₃-C₆ cycloalkyl ring and R^{y} is H, halo, or C₁-C₆ alkyl; and
B is C₃-C₈ cycloalkyl, 5-13 membered heterocycloalkyl, aryl or heteroaryl, and B is substituted by 0, 1, 2, or 3 substituents, each independently selected from C₁-C₃ alkyl, —O(C₁-C₆ alkyl), —(C₁-C₆ alkylenyl)-O(C₁-C₃ alkyl), —(C₁-C₆ alkylenyl)-O(C₁-C₆ alkylenyl)-O(C₁-C₃ alkyl), halogen, heteroalkyl, hydroxyl, 5-9 membered heterocycloalkyl, —O(C₃-C₆ cycloalkyl), C₃-C₆ cycloalkyl, 5-9 membered heteroaryl, 5-9 membered aryl, —O(C₆-C₁₀ aryl), and phosphorus atom substituted with oxo and/or C₁-C₃ alkyl, wherein one or more of the hydrogen atoms of the C₁-C₃ alkyl, C₁—C₆ alkyl or heteroalkyl may be substituted with deuterium or fluoro and wherein the 5-9 membered heterocycloalkyl, 5-9 membered heteroaryl or 5-9 membered aryl is substituted with 0, 1, 2, or 3 substituents independently selected from —C₁-C₃ alkyl, —C₁-C₃ haloalkyl, —C₃-C₆ cycloalkyl, and oxo;
R⁸ is C₁-C₆ alkyl, 5-6 membered heterocycloalkyl, heteroaryl, or aryl, and R⁸ is substituted by 0, 1, 2, or 3 substituents, each independently selected from C₁-C₃ alkyl, C₃-C₆ cycloalkyl, O(C₃-C₆ cycloalkyl), phenyl, halogen, SO₂CH₃ and oxo, wherein one or more of the hydrogen atoms of the C₁-C₃ alkyl or C₃-C₆ cycloalkyl may be substituted with fluoro or chloro; or
R⁸ is —R^{8a}—R^{8b} and R^{8a} is

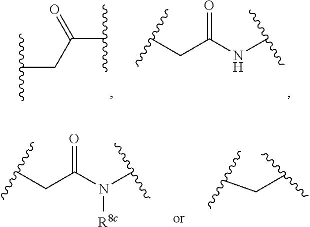

and R^{8b} is aryl, heteroaryl, 4-10 membered heterocycloalkyl, C₃-C₁₀ cycloalkyl, wherein R^{bb} is substituted by 0, 1, 2 or 3 substituents, each independently selected from C₁-C₃ alkyl, halogen, and oxo, wherein one or more of the hydrogen atoms of the C₁-C₃ alkyl may be substituted with fluoro or chloro; optionally, wherein if R^{8a} is

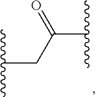 [SMILES: *CC(*)=O]

then R^{8b} is heteroaryl or 4-10 membered heterocycloalkyl with at least one nitrogen, and R^{8a} forms a bond with the nitrogen of the heteroaryl or 4-10 membered heterocycloalkyl;
wherein R^{8c}, when present, is C₁-C₆ alkyl, substituted with 0, 1, 2, or 3 substituents independently selected from halogen, C₃-C₆ cycloalkyl, phenyl or 3-8 membered heterocycloalkyl;
optionally, wherein at least one of (a)-(h) is true:
(a) R⁸ is other than optionally substituted C₆₋₁₀ aryl and optionally substituted or 5 to 10 membered heteroaryl;
(b) Y is other than

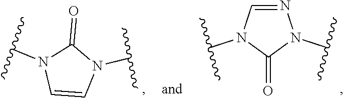 [SMILES: *n1ccn(*)c1=O ; *n1cnn(*)c1=O]

wherein the left [symbol] on Y is bound to the pyrazole moiety and the right [symbol] on Y is bound to B;
(c) R¹ is other than optionally substituted 3 to 10 membered heterocycloalkyl and 5 to 10 membered heteroaryl;
(d) n is 1 and R⁶ is C₁-C₆ alkyl and R⁷ is C₁-C₆ alkyl, or
(e) n is 1 and R⁶ and R⁷ together with the atom to which they are attached, form a C₃-C₆ cycloalkyl ring or a 3-6 membered heterocycloalkyl ring;
(f) n is 2;
(g) Y is other than

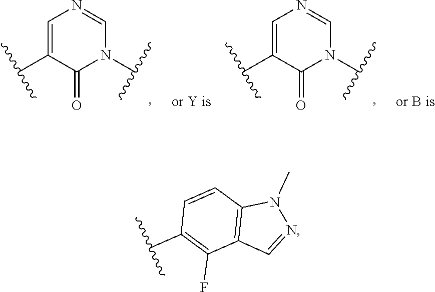 [SMILES: *c1cncn(*)c1=O ; *c1cncn(*)c1=O ; *c1ccc2c(cnn2C)c1F]

wherein the left [symbol] on Y is bound to the pyrazole moiety and the right [symbol] on Y is bound to B; and
(h) Y is other than

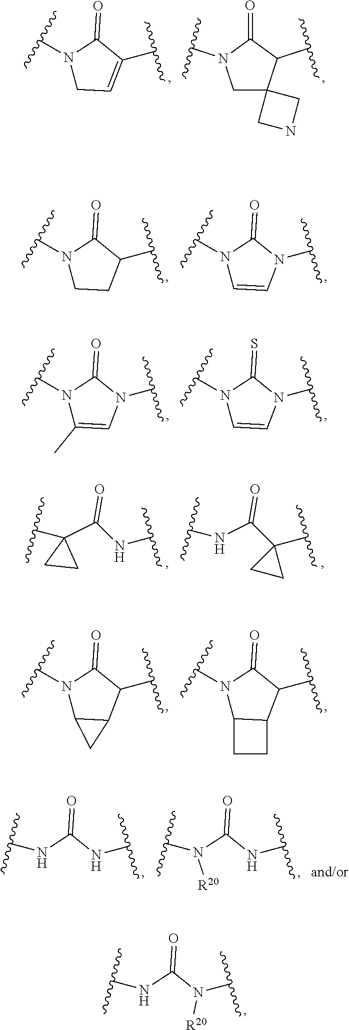

wherein the left [symbol] is bound to the pyrazole moiety and the right [symbol] is bound to B and R²⁰ is selected from C₁-C₃ alkyl or cyclopropyl.

The invention also includes compositions comprising a compound of Formula I or a pharmaceutically acceptable salt thereof, and a pharmaceutically acceptable carrier. The invention further includes methods for treating obesity by administration of a compound of Formula I, or a pharmaceutically acceptable salt thereof, to a patient in need thereof, or by administration of a pharmaceutical composition comprising a compound of Formula I or its salt and a pharmaceutically acceptable carrier.

The summary of the technology described above is non-limiting and other features and advantages of the technology will be apparent from the following detailed description, and from the claims.

### DETAILED DESCRIPTION

As noted above, the present invention includes compounds of Formula I, wherein the compounds are GLP-1 receptor agonists suitable for use for the treatment of obesity.

Unless otherwise designated, reference to a compound of the Formula I, as used herein, includes compounds of the Formulas I, Ia, II, IIa, III, IV, IVa, IVb, V, Va, Vb, VI, VIa, VIb, VII, VIIa, VIII, IX, IXa, IXb, X, Xa and Xb.

The present invention encompasses for each of the various embodiments of the compounds of the invention described herein, including those of Formula I, and the various embodiments thereof and the compounds of the examples, all forms of the compounds such as, for example, any solvates, hydrates, stereoisomers, and tautomers of said compounds and of any pharmaceutically acceptable salts thereof, unless otherwise indicated. Additionally, in the examples described herein, the compounds of the invention may be depicted in the salt form. In such cases, it is to be understood that the compounds of the invention include the free acid or free base forms of such salts, and any pharmaceutically acceptable salt of said free acid or free base forms. In addition, in instances where an acidic group such as tetrazole and a basic group such as an amine are present within the same compound, these compounds may be drawn herein for convenience as the free acid and base forms but it should be understood that these can also be alternatively depicted in their zwitterionic forms in which the tetrazole bears a negative charge and the amine bears a positive charge, which are also included as compounds of the invention.

In one aspect, the present invention includes compounds of Formula I:

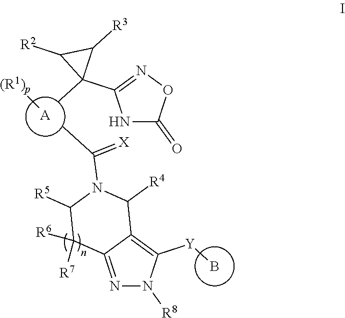

or a pharmaceutically acceptable salt thereof, wherein R¹, R², R³, R⁴, R⁵, R⁶, R⁷, R⁸, n, p, A, X, Y and B are as defined herein for the compounds of Formula (I) in embodiment no. 1 (i.e., as defined in the Summary of the Invention); wherein the compounds may be suitable for use for the treatment of obesity.

In embodiment no. 2, the present invention provides a compound of Formula I, or a pharmaceutically acceptable salt thereof, wherein:
R¹ is halogen, CN, OH, aminoalkyl, hydroxyalkyl, diaminoalkyl, dihydroxyalkyl, C₁-C₆ alkyl, heteroalkyl, C₃-C₈ cycloalkyl, 3-10 membered heterocycloalkyl, —C≡C-(3-10 membered heterocycloalkyl), —C≡C—(C₃-C₈ cycloalkyl), or -phenyl-(C₃-C₈ cycloalkyl), wherein R¹ is substituted with 0 to 5 substituents independently selected from halogen, CN, OH, C₁-C₆ alkyl, C₁-C₆ alkyl substituted with one or more halogen atoms, or C₁-C₆ alkyloxy;
R² is H or methyl;
R³ is H or methyl;
R⁴ is hydrogen or C₁-C₆ alkyl;
R⁵ is hydrogen or C₁-C₆ alkyl; or
R⁴ and R⁵, together with the atom which they are attached, or R⁴ and R⁶, together with the atom which they are attached, form a bridged C₇-C₈ heterocyclic ring fused to the pyrazole; each R⁶ is independently H, halo, or C₁-C₆ alkyl and each R⁷ is independently H, halo, or C₁-C₆ alkyl, or
R⁶ and R⁷ together with the atom to which they are attached, form a C₃-C₆ cycloalkyl ring or a 3-6 membered heterocycloalkyl ring, provided that if there are two R⁶ and R⁷ then only one set forms a ring and the other R⁶ and R⁷ is each independently H or C₁-C₆ alkyl;
n is 1 or 2;
p is 1, 2, 3, 4 or 5;
R⁴, R⁵, R⁶ and R⁷ are each independently substituted with 0 to 2 substituents independently selected from halogen, NH₂, aminoalkyl, diaminoalkyl, C₁-C₆ alkyl, and C₁-C₆ alkyl substituted with one or more halogen atoms, C₁-C₆ alkoxy, hydroxy, oxo, CONR^{w1}R^{w2}, SO₂N R^{w3}R^{w4}, SO₂R^{w5}, N(R^{w6})COR^{w7}, NR^{w8}SO₂R^{w9}, wherein R^{w1}, R^{w2}, R^{w3}, R^{w4}, R^{w5}, R^{w6}, R^{w7},R^{w8}, and R^{w9}, are each individually selected from H and C₁-C₆ alkyl;
A is 5-10 membered heteroaryl or C₅-C₁₀ aryl;
X is O, S, or NR^{XX} wherein R^{XX} is H or C₁-C₃ alkyl, wherein C₁-C₃ alkyl is substituted with 0, 1, or 2 substituents selected from halogen and C₃-C₆ cycloalkyl;
Y is

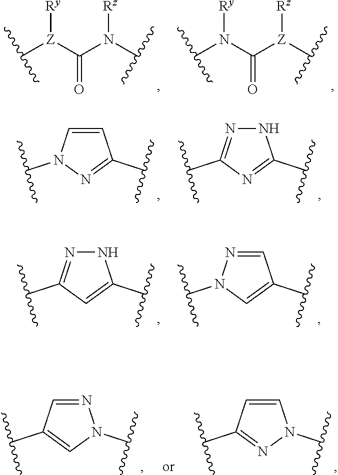

wherein the left [symbol] is bound to the pyrazolopyridine structure and the right [symbol] is bound to B,
Z is C, CR^{x} or N; [symbol]
R^{x} is H, halo, or C₁-C₆ alkyl;
R^{y}, and R^{z}, together with the atom which they are attached and optionally R^{x}, form a 5-8 membered heterocycloalkyl ring or a 5-6 membered heteroaryl; or
R^{y}, and R^{z} are each independently H, halo, or C₁-C₆ alkyl; or
R^{x} and R^{y}, together with the carbon to which they are attached, form a spiro C₃-C₆ cycloalkyl ring and R^{z} is H, halo, or C₁-C₆ alkyl; or
R^{x} and R^{z}, together with the carbon to which they are attached, form a spiro C₃-C₆ cycloalkyl ring and R^{y} is H, halo, or C₁-C₆ alkyl; and
B is C₃-C₈ cycloalkyl, 5-13 membered heterocycloalkyl, aryl or heteroaryl, and B is substituted by 0, 1, 2, or 3 substituents, each independently selected from C₁-C₃ alkyl, halogen, heteroalkyl, 6-9 membered heterocycloalkyl, —O(C₃-C₆ cycloalkyl), C₃-C₆ cycloalkyl, 5-9 membered heterocycloalkyl, —O(C₆-C₁₀ aryl), and phosphorus atom substituted with oxo and/or C₁-C₃ alkyl, wherein one or more of the hydrogen atoms of the C₁-C₃ alkyl or heteroalkyl may be substituted with fluoro and wherein the 5-9 membered heterocycloalkyl may be unsubstituted or substituted with 1, 2, or 3 substituents independently selected from —C₁-C₃ alkyl, —C₁-C₃ haloalkyl, —C₃-C₆ cycloalkyl, and oxo;
R⁸ is C₁-C₆ alkyl, 5-6 membered heterocycloalkyl, heteroaryl, or aryl, and R⁸ is substituted by 0, 1, 2, or 3 substituents, each independently selected from C₁-C₃ alkyl, C₃-C₆ cycloalkyl, O(C₃-C₆ cycloalkyl), phenyl, halogen, SO₂CH₃ and oxo, wherein one or more of the hydrogen atoms of the C₁-C₃ alkyl or C₃-C₆ cycloalkyl may be substituted with fluoro or chloro; or R⁸ is R^{8a}R^{8b} and R^{8a} is

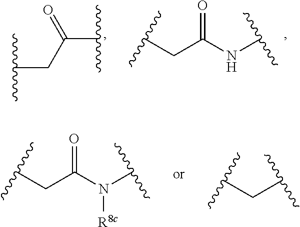

and R^{8b} is aryl, heteroaryl, 4-10 membered heterocycloalkyl, C₃-C₁₀ cycloalkyl, wherein R^{8b} is substituted by 0, 1, 2 or 3 substituents, each independently selected from C₁-C₃ alkyl, halogen, and oxo, wherein one or more of the hydrogen atoms of the C₁-C₃ alkyl may be substituted with fluoro or chloro; wherein if R^{8a} is

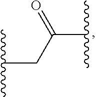 [SMILES: *CC(*)=O]

then R^{8b} is heteroaryl or 4-10 membered heterocycloalkyl with at least one nitrogen, and R^{8b} forms a bond with the nitrogen of the heteroaryl or 4-10 membered heterocycloalkyl;
wherein R^{8c}, when present, is C₁-C₆ alkyl, substituted with 0, 1, 2, or 3 substituents independently selected from halogen, C₃-C₆ cycloalkyl, phenyl or 3-8 membered heterocycloalkyl;
optionally, wherein at least one of (a)-(f) is true:
(a) R⁸ is other than optionally substituted C₆₋₁₀ aryl and optionally substituted or 5 to 10 membered heteroaryl;
(b) Y is other than

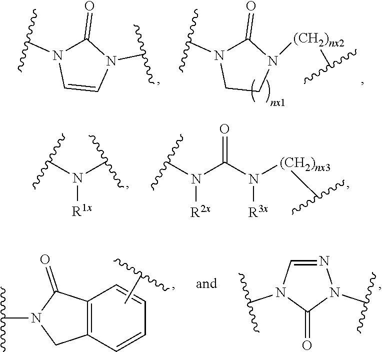

wherein the left [symbol] is bound to the pyrazolopyridine structure and the right [symbol] is bound to B, and
nx1 is an integer of 1 to 3; [symbol]
nx2 and nx3 are independently an integer of 1 to 10;
R^{1x} is hydrogen, C₁-C₆ alkyl, or (C₁-C₆ alkyl)carbonyl;
R^{2x} and R^{3x} are independently hydrogen or C₁-C₆ alkyl;
(c) R¹ is other than optionally substituted 3 to 12 membered heterocycloalkyl and 5 to 10 membered heteroaryl;
(d) n is 1 and R⁶ is C₁-C₆ alkyl and R⁷ is C₁-C₆ alkyl, or
(e) n is 1 and R⁶ and R⁷ together with the atom to which they are attached, form a C₃-C₆ cycloalkyl ring or a 3-6 membered heterocycloalkyl ring; and
(f) n is 2.

In embodiment no. 3, the present invention provides a compound of Formula I, or a pharmaceutically acceptable salt thereof, wherein the compound of Formula I has the Formula Ia,

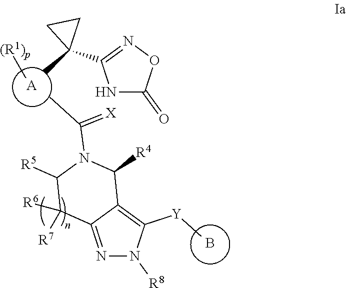

and R¹, R⁴, R⁵, R⁶, R⁷, R⁸, n, p, A, X, Y and B are as defined in embodiment no. 1.

In embodiment no. 4, the present invention provides a compound of Formula I, or a pharmaceutically acceptable salt thereof, wherein the compound of Formula I has the Formula II

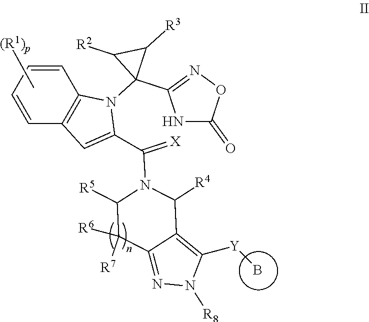

and R¹, R², R³, R⁴, R⁵, R⁶, R⁷, R⁸, n, p, X, Y and B are as defined in embodiment no. 1.

In embodiment no. 5, the present invention provides a compound of Formula IIa, or a pharmaceutically acceptable salt thereof, wherein the compound of Formula I has the Formula IIa,

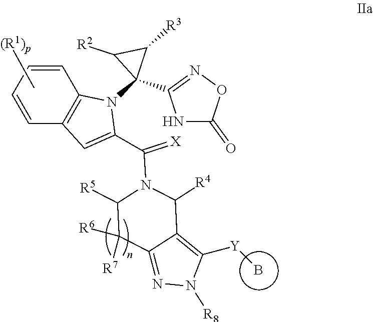

and R¹, R², R³, R⁴, R⁵, R⁶, R⁷, R¹, n, p, X, Y and B are as defined in embodiment no. 1.

In embodiment no. 6, the present invention provides a compound of Formula I, or a pharmaceutically acceptable salt thereof, wherein the compound of Formula I has the Formula III,

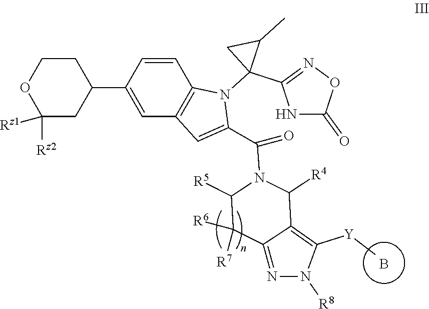

and R¹, R⁴, R⁵, R⁶, R⁷, R¹, n, X, Y and B are as defined in embodiment no. 1 and R^{z1} and R^{z2} are as defined below.

In embodiment no. 7, the present invention provides a compound of Formula I, or a pharmaceutically acceptable salt thereof, wherein the compound of Formula I has the Formula IV,

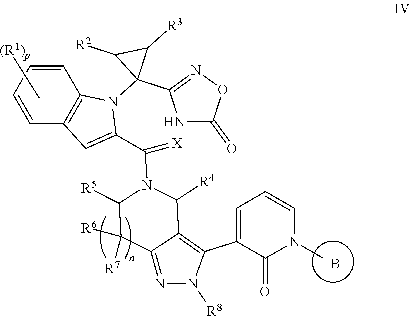

and R¹, R², R³, R⁴, R⁵, R⁶, R⁷, R⁸, n, p, X, and B are as defined in embodiment no. 1.

In embodiment no. 8, the present invention provides a compound of Formula I, or a pharmaceutically acceptable salt thereof, wherein the compound of Formula I has the Formula IVa,

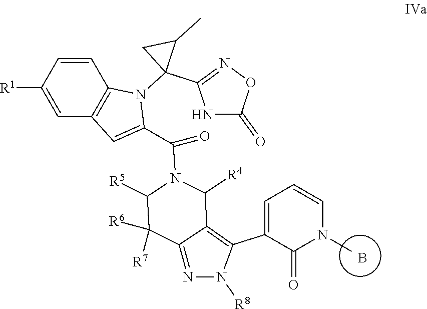

and R¹, R⁴, R⁵, R⁶, R⁷, R⁸, and B are as defined in embodiment no. 1.

In embodiment no. 9, the present invention provides a compound of Formula I, or a pharmaceutically acceptable salt thereof, wherein the compound of Formula I has the Formula IVb,

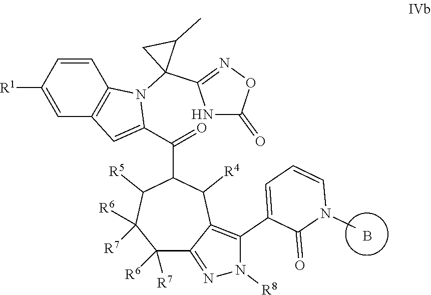

and R¹, R⁴, R⁵, R⁶, R⁷, R⁸, and B are as defined in embodiment no. 1.

In embodiment no. 10, the present invention provides a compound of Formula I, or a pharmaceutically acceptable salt thereof, wherein the compound of Formula I has the Formula V,

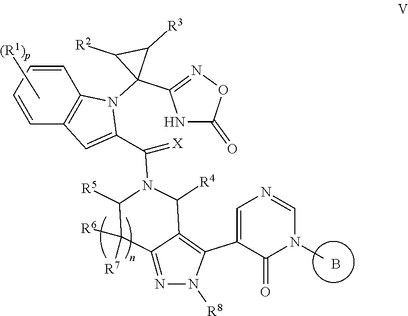

and R¹, R², R³, R⁴, R⁵, R⁶, R⁷, R⁸, n, p, X, and B are as defined in embodiment no. 1.

In embodiment no. 11, the present invention provides a compound of Formula I, or a pharmaceutically acceptable salt thereof, wherein the compound of Formula I has the Formula Va,

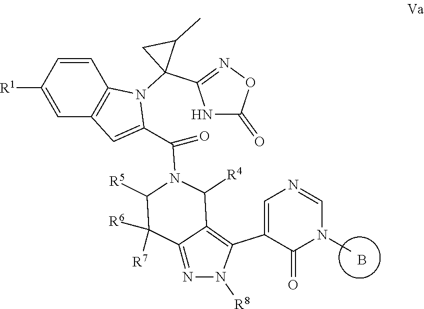

and R¹, R⁴, R⁵, R⁶, R⁷, R⁸, and B are as defined in embodiment no. 1.

In embodiment no. 12, the present invention provides a compound of Formula I, or a pharmaceutically acceptable salt thereof, wherein the compound of Formula I has the Formula Vb,

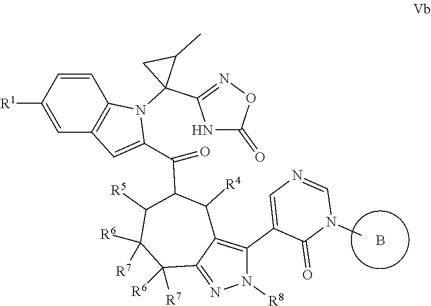

and R¹, R⁴, R⁵, R⁶, R⁷, R⁸, and B are as defined in embodiment no. 1.

In embodiment no. 13, the present invention provides a compound of Formula I, or a pharmaceutically acceptable salt thereof, wherein the compound of Formula I has the Formula VI,

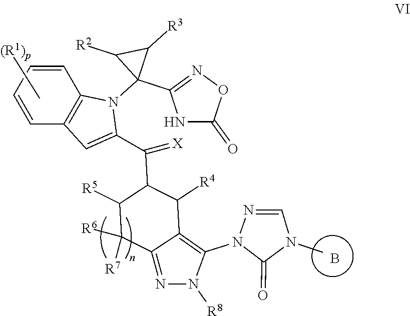

and R¹, R², R³, R⁴, R⁵, R⁶, R⁷, R⁸, n, p, X, and B are as defined in embodiment no. 1.

In embodiment no. 14, the present invention provides a compound of Formula I, or a pharmaceutically acceptable salt thereof, wherein the compound of Formula I has the Formula VIa,

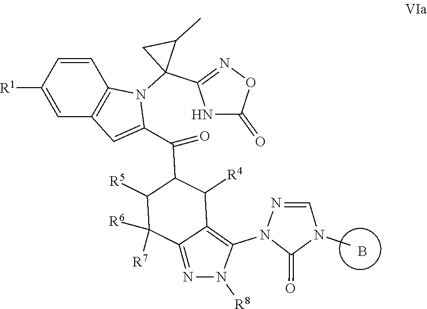

and R¹, R⁴, R⁵, R⁶, R⁷, R⁸, and B are as defined in embodiment no. 1.

In embodiment no. 15, the present invention provides a compound of Formula I, or a pharmaceutically acceptable salt thereof, wherein the compound of Formula I has the Formula VIb,

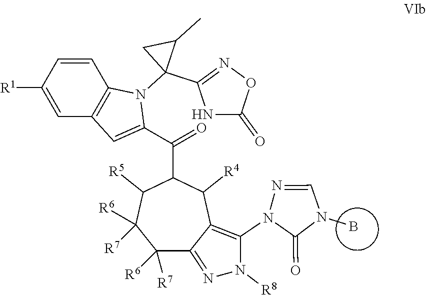

and R¹, R⁴, R⁵, R⁶, R⁷, R⁸, and B are as defined in embodiment no. 1.

In embodiment no. 16, the present invention provides a compound of Formula I, or a pharmaceutically acceptable salt thereof, wherein the compound of Formula I has the Formula VII,

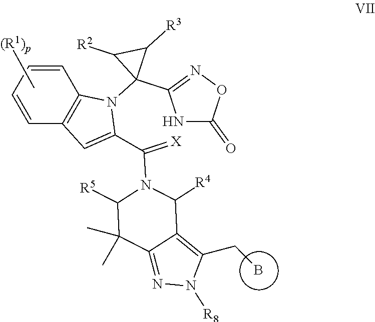

and R¹, R², R³, R⁴, R⁵, R⁸, p, A, X, Y and B are as defined in embodiment no. 1.

In embodiment no. 17, the present invention provides a compound of Formula I, or a pharmaceutically acceptable salt thereof, wherein the compound of Formula I has the Formula VIIa,

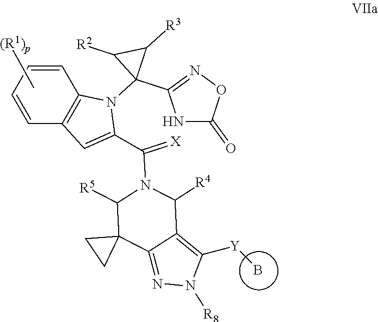

and R¹, R², R³, R⁴, R⁵, R⁸, p, A, X, Y and B are as defined in embodiment no. 1.

In embodiment no. 18, the present invention provides a compound of Formula I, or a pharmaceutically acceptable salt thereof, wherein the compound of Formula I has the Formula VIII,

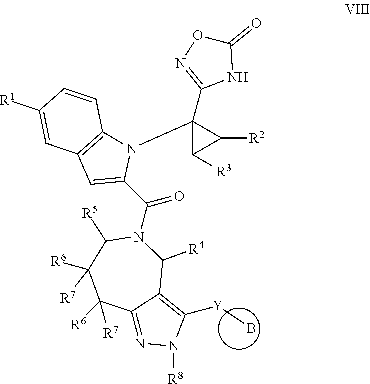

and R¹, R², R³, R⁴, R⁵, R⁶, R⁷, R⁸, Y and B are as defined in embodiment no. 1.

In embodiment no. 19, the present invention provides a compound of Formula I, or a pharmaceutically acceptable salt thereof, wherein the compound of Formula I has the Formula IX,

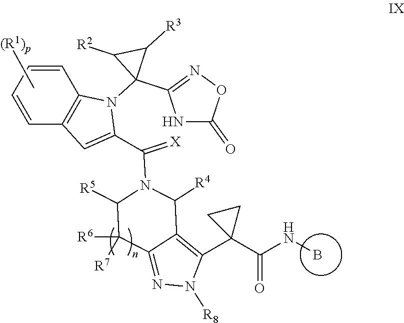

and R¹, R², R³, R⁴, R⁵, R⁶, R⁷, R⁸, n, p, X, and B are as defined in embodiment no. 1.

In embodiment no. 20, the present invention provides a compound of Formula I, or a pharmaceutically acceptable salt thereof, wherein the compound of Formula I has the Formula IXa,

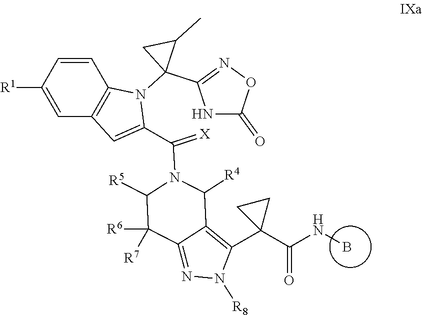

and R¹, R⁴, R⁵, R⁶, R⁷, R⁸, and B are as defined in embodiment no. 1.

In embodiment no. 21, the present invention provides a compound of Formula I, or a pharmaceutically acceptable salt thereof, wherein the compound of Formula I has the Formula

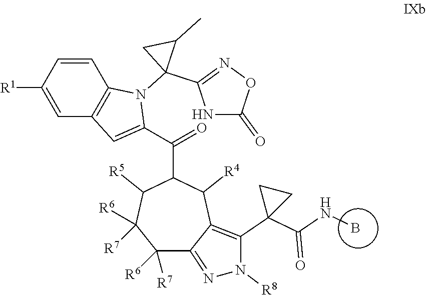

and R¹, R⁴, R⁵, R⁶, R⁷, R⁸, and B are as defined in embodiment no. 1.

In embodiment no. 22, the present invention provides a compound of Formula I, or a pharmaceutically acceptable salt thereof, wherein the compound of Formula I has the Formula X,

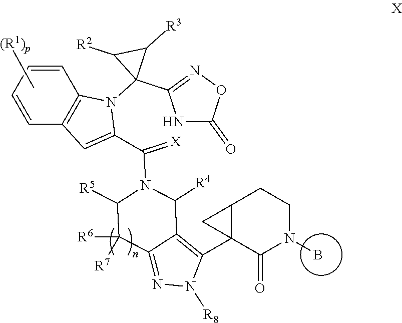

and R¹, R², R³, R⁴, R⁵, R⁶, R⁷, R⁸, n, p, X, and B are as defined in embodiment no. 1.

In embodiment no. 23, the present invention provides a compound of Formula I, or a pharmaceutically acceptable salt thereof, wherein the compound of Formula I has the Formula Xa,

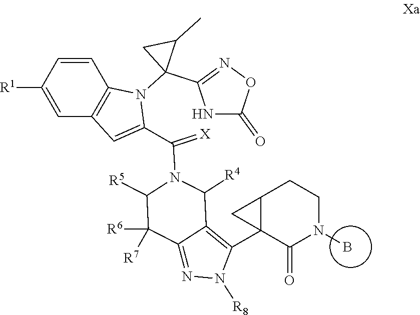

and R¹, R⁴, R⁵, R⁶, R⁷, R⁸, and B are as defined in embodiment no. 1.

In embodiment no. 24, the present invention provides a compound of Formula I, or a pharmaceutically acceptable salt thereof, wherein the compound of Formula I has the Formula Xb,

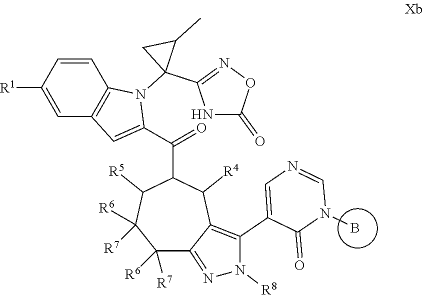

and R¹, R⁴, R⁵, R⁶, R⁷, R⁸, and B are as defined in embodiment no. 1.

In embodiment no. 25, the present invention provides a compound of Formula I as set forth in any one of embodiment nos. 1-3, or a pharmaceutically acceptable salt thereof, wherein A is 6-9 membered heteroaryl or C₆-C₉ aryl.

In embodiment no. 26, the present invention provides a compound of Formula I as set forth in any one of embodiment nos. 1-3 or a pharmaceutically acceptable salt thereof, wherein A is

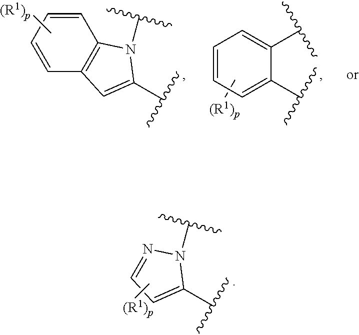

In embodiment no. 27, the present invention provides a compound of Formula I as set forth in any one of embodiment nos. 1-5 and 7-24, or a pharmaceutically acceptable salt thereof, wherein

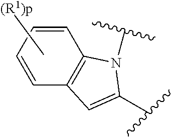

is

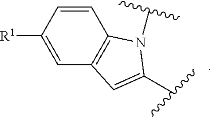

In embodiment no. 28, the present invention provides a compound of Formula I as set forth in any one of embodiment nos. 1-5 and 7-24, or a pharmaceutically acceptable salt thereof, wherein A is

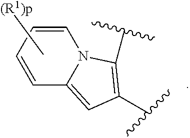

In embodiment no. 29, the present invention provides a compound of Formula I as set forth in any one of embodiment nos. 1-5 and 7-24, or a pharmaceutically acceptable salt thereof, wherein A is

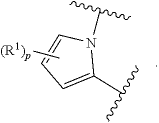

In embodiment no. 30, the present invention provides a compound of Formula I as set forth in embodiment no. 28, or a pharmaceutically acceptable salt thereof, wherein

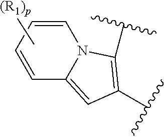

is

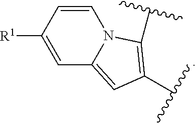

In embodiment no. 31, the present invention provides a compound of Formula I as set forth in embodiment no. 29, or a pharmaceutically acceptable salt thereof, wherein

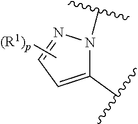

is

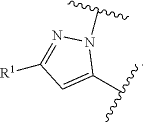

In embodiment no. 32, the present invention provides a compound of Formula I as set forth in embodiment no. 31, or a pharmaceutically acceptable salt thereof, wherein

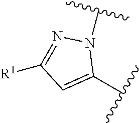

is

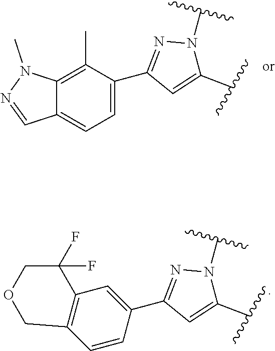 [SMILES: *c1cc(-c2ccc3cnn(C)c3c2C)nn1* ; *c1cc(-c2ccc3c(c2)C(F)(F)COC3)nn1*]

In embodiment no. 33 the present invention provides a compound of Formula I as set forth in any one of embodiment nos. 1-24, or a pharmaceutically acceptable salt thereof, wherein R¹ is 6-10 membered heterocycloalkyl or C₃-C₈ cycloalkyl, wherein R¹ is substituted with 0 to 5 substituents independently selected from a halogen, C₁-C₆ alkyl, C₁-C₆ alkyl substituted with one or more halogen atoms, and C₁-C₆ alkoxy.

In embodiment no. 34, the present invention provides a compound of Formula I as set forth in any one of embodiment nos. 1-24, or a pharmaceutically acceptable salt thereof, wherein R¹ is 6-10 membered heterocycloalkyl wherein R¹ is substituted with 0 to 2 substituents independently selected from C₁-C₆ alkyl.

In embodiment no. 35, the present invention provides a compound of Formula I as set forth in any one of embodiment nos. 1-24, or a pharmaceutically acceptable salt thereof, wherein R¹ is 6-10 membered heterocycloalkyl, wherein R¹ is substituted with two methyl groups.

In embodiment no. 36, the present invention provides a compound of Formula I as set forth in any one of embodiment nos. 1-24, or a pharmaceutically acceptable salt thereof, wherein R¹ is 6-10 membered heterocycloalkyl containing one or two oxygen atoms as ring members, wherein R¹ is substituted with 0 to 3 substituents independently selected from OH, C₁-C₆ alkyl, or C₁-C₆ alkyloxy.

In embodiment no. 37, the present invention provides a compound of Formula I as set forth in any one of embodiment nos. 1-24, or a pharmaceutically acceptable salt thereof, wherein R¹ is C₃-C₈ cycloalkyl, wherein R¹ is substituted with 0 to 2 substituents independently selected from OH and C₁-C₆ alkoxy.

In embodiment no. 38, the present invention provides a compound of Formula I as set forth in any one of embodiment nos. 1-24, or a pharmaceutically acceptable salt thereof, wherein R¹ is

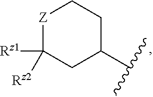

Z is O or CH(OCH₃); R^{z1} is H or C₁-C₆ alkyl and R^{z2} is H or C₁-C₆ alkyl.

In embodiment no. 39, the present invention provides a compound of Formula I as set forth in any one of embodiment nos. 1-24, or a pharmaceutically acceptable salt thereof, wherein R¹ is

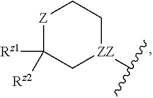

Z is O or CH(OCH₃); R^{z1} is H, halogen, or C₁-C₆ alkyl and R^{z2} is H, halogen or C₁-C₆ alkyl, and ZZ is N.

In embodiment no. 40, the present invention provides a compound of Formula I as set forth in any one of embodiment nos. 1-24, or a pharmaceutically acceptable salt thereof, wherein R¹ is tetrahydropyranyl, wherein R¹ is substituted with 0 or 2 methyl groups.

In embodiment no. 41, the present invention provides a compound of Formula I as set forth in any one of embodiment nos. 1-5 and 7-24, or a pharmaceutically acceptable salt thereof, wherein R¹ is

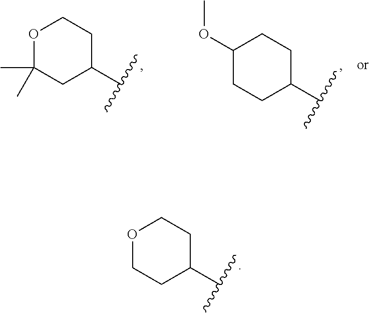

In embodiment no. 42, the present invention provides a compound of Formula I as set forth in any one of embodiment nos. 1-5and 7-24, or a pharmaceutically acceptable salt thereof, wherein R¹ is

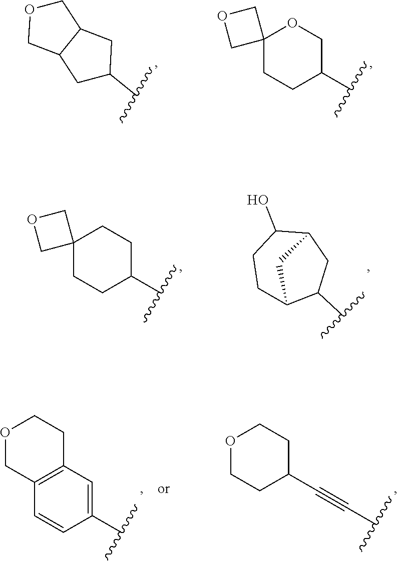 [SMILES: *C1CC2COCC2C1 ; *C1CCC2(COC2)OC1 ; *C1CCC2(CC1)COC2 ; *C1C[C@H]2C[C@@H]1CCC2O ; *c1ccc2c(c1)CCOC2 ; *C#CC1CCOCC1]

and R¹ is substituted with 0 to 3 substituents independently selected from halogen, C₁-C₆ alkyl, C₁-C₆ alkyl substituted with one or more halogen atoms, and C₁-C₆ alkoxy.

In embodiment no. 43, the present invention provides a compound of Formula I as set forth in any one of embodiment nos. 1-5 and 7-18, or a pharmaceutically acceptable salt thereof, wherein R¹ is

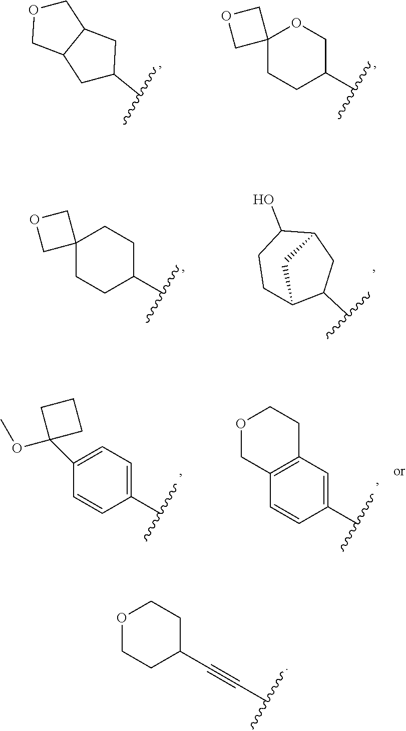

In embodiment no. 44, the present invention provides a compound of Formula I as set forth in any one of embodiment nos. 1-5 and 7-24, or a pharmaceutically acceptable salt thereof, wherein R¹ is

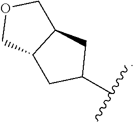 [SMILES: *C1C[C@@H]2COC[C@H]2C1]

In embodiment no. 45, the present invention provides a compound of Formula I as set forth in any one of embodiment nos. 1-5 and 7-24, or a pharmaceutically acceptable salt thereof, wherein R¹ is

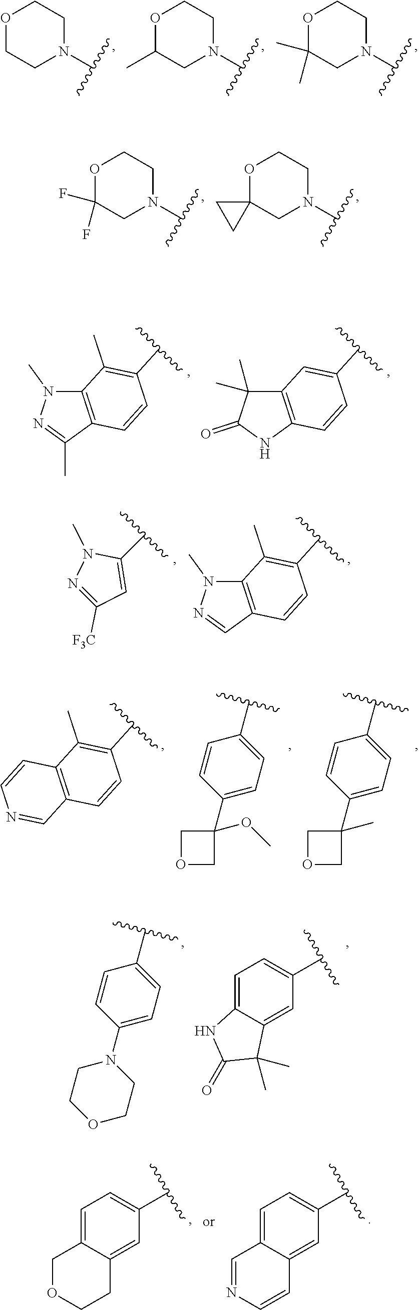

In a sub-embodiment of embodiment no. 45, R¹ is

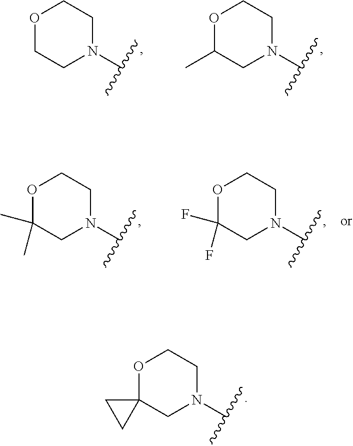

In embodiment no. 46, the present invention provides a compound of Formula I as set forth in any one of embodiment nos. 1-2, 4-5, 7, 10, 13, and 16-18, 20, 23 and 25-45, or a pharmaceutically acceptable salt thereof, wherein R² is H and R³ is H.

In embodiment no. 47, the present invention provides a compound of Formula I as set forth in any one of embodiment nos. 1-2, 4-5, 7, 10, 13, and 16-18, 20, 23 and 25-45, or a pharmaceutically acceptable salt thereof, wherein R² is H and R³ is methyl.

In embodiment no. 48, the present invention provides a compound of Formula I as set forth in any one of embodiment nos. 1-47, or a pharmaceutically acceptable salt thereof, wherein
R⁴ is hydrogen, methyl, or ethyl;
R⁵ is hydrogen or methyl;
R⁴ and R⁵, together with the atom which they are attached, or R⁴ and R⁶, together with the atom which they are attached, form a bridged C₇₋₈ heterocyclic ring fused to the pyrazole; and
R⁶ is H, halo, or methyl, and R⁷ is H, halo, or methyl, or
R⁶ and R⁷ together with the atom to which they are attached, form a cyclopropyl ring or a cyclobutyl ring provided that if there are two R⁶ and R⁷ then only one set forms a ring and the other R⁶ and R⁷ is each independently H or methyl.

In embodiment no. 49, the present invention provides a compound of Formula I as set forth in embodiment no. 48, or a pharmaceutically acceptable salt thereof, wherein R⁴ is hydrogen or methyl.

In embodiment no. 50, the present invention provides a compound of Formula I as set forth in any one of embodiment nos. 1-48, or a pharmaceutically acceptable salt thereof, wherein R⁴ is methyl.

In embodiment no. 51, the present invention provides a compound of Formula I as set forth in any one of embodiment nos. 1-50, or a pharmaceutically acceptable salt thereof, wherein R⁵ is H.

In embodiment no. 52, the present invention provides a compound of Formula I as set forth in any one of embodiment nos. 1-48, or a pharmaceutically acceptable salt thereof, wherein R⁴ is methyl and R⁵ is H.

In embodiment no. 53, the present invention provides a compound of Formula I as set forth in any one of embodiment nos. 1-7, 10, 13, and 25-52, or a pharmaceutically acceptable salt thereof, wherein R⁶ is methyl, R⁷ is methyl, and n=1.

In embodiment no. 54, the present invention provides a compound of Formula I as set forth in any one of embodiment nos. 1-7, 10, 13, and 25-52, or a pharmaceutically acceptable salt thereof, wherein R⁶ is halo, R⁷ is halo, and n=1.

In embodiment no. 55, the present invention provides a compound of Formula I as set forth in any one of embodiment nos. 1-7, 10, 13, and 25-52, or a pharmaceutically acceptable salt thereof, wherein R⁶ is fluoro, R⁷ is fluoro, and n=1,

In embodiment no. 56, the present invention provides a compound of Formula I as set forth in any one of embodiment nos. 1-15, 18-48 and 51, or a pharmaceutically acceptable salt thereof, wherein R⁴ and R⁶, together with the atom to which they are attached, form a bridged C₇₋₈ heterocyclic ring fused to the pyrazole.

In embodiment no. 57, the present invention provides a compound of Formula I as set forth in any one of embodiment nos. 1-7, 10, 13, 25-48 and 5118-34 and 36, or a pharmaceutically acceptable salt thereof, wherein R⁴ and R⁶, together with the atom to which they are attached, form a bridged C₇₋₈ heterocyclic ring fused to the pyrazole, and n=1.

In embodiment no. 58, the present invention provides a compound of Formula I as set forth in any one of embodiment nos. 1-15, and 25-49, or a pharmaceutically acceptable salt thereof, wherein R⁴ and R⁵, together with the atom to which they are attached, form a bridged C₇₋₈ heterocyclic ring fused to the pyrazole.

In embodiment no. 59, the present invention provides a compound of Formula I as set forth in any one of embodiment nos. 1-7, 10, 13, and 25-49, or a pharmaceutically acceptable salt thereof, wherein R⁴ and R⁵, together with the atom to which they are attached, form a bridged C₇₋₈ heterocyclic ring fused to the pyrazole, and n=1.

In embodiment no. 60, the present invention provides a compound of Formula I as set forth in any one of embodiment nos. 1-7, 10, 13, 25-52, 56 and 58, or a pharmaceutically acceptable salt thereof, wherein n is 1.

In embodiment no. 61, the present invention provides a compound of Formula I as set forth in any one of embodiment nos. 1-7, 10, 13, 25-52, 56 and 58, or a pharmaceutically acceptable salt thereof, wherein n is 2.

In embodiment no. 62, the present invention provides a compound of Formula I as set forth in any one of embodiment nos. 1-47, or a pharmaceutically acceptable salt thereof, wherein

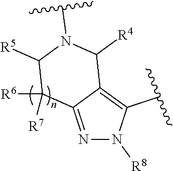

is:

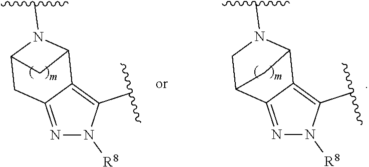

In embodiment no. 63, the present invention provides a compound of Formula I as set forth in any one of embodiment nos. 1-47, or a pharmaceutically acceptable salt thereof, wherein

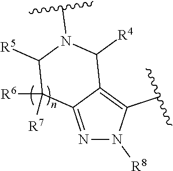

is:

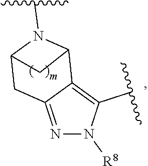

and m is 2.

In embodiment no. 64, the present invention provides a compound of Formula I as set forth in any one of embodiment nos. 1-47, or a pharmaceutically acceptable salt thereof, wherein

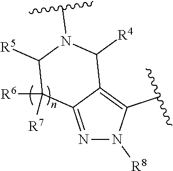

is:

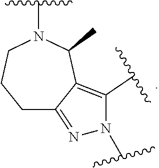 [SMILES: *c1c2c(nn1*)CCCN(*)[C@H]2C]

In embodiment no. 65, the present invention provides a compound of Formula I as set forth in any one of embodiment nos. 1-47, or a pharmaceutically acceptable salt thereof, wherein

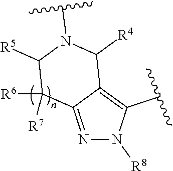

is:

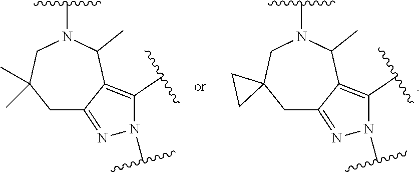 [SMILES: *c1c2c(nn1*)CC(C)(C)CN(*)C2C ; *c1c2c(nn1*)CC1(CC1)CN(*)C2C]

In embodiment no. 66, the present invention provides a compound of Formula I as set forth in any one of embodiment nos. 1-47, or a pharmaceutically acceptable salt thereof, wherein

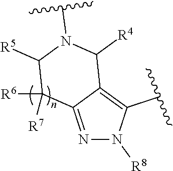

is:

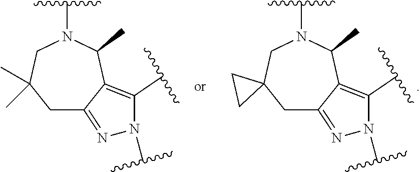 [SMILES: *c1c2c(nn1*)CC(C)(C)CN(*)[C@H]2C ; *c1c2c(nn1*)CC1(CC1)CN(*)[C@H]2C]

In embodiment no. 67, the present invention provides a compound of Formula I as set forth in embodiment no. 66, or a pharmaceutically acceptable salt thereof, wherein

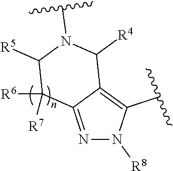

is:

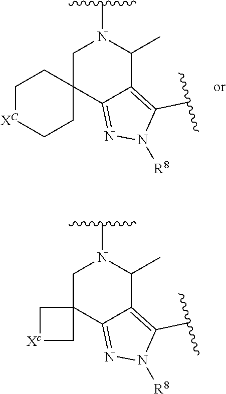

and X^{c} is O or NR⁹ wherein t is 1, 2, 3, 4 or 5 and R⁹ is H, C₁-C₆ alkyl, SO₂—(C₁-C₆ alkyl), ((C₁-C₆ alkylenyl)O)ₜ(C₁-C₆ alkyl), —(C₁-C₆ alkylenyl)(C₃-C₈cycloalkyl), —(C)(O)(C₁-C₆ alkyl), —(CH₂)aryl, C₃-C₈ cycloalkyl, —(CH₂)(C₃-C₈cycloalkyl), or C₁-C₆ alkyl wherein one or more of the hydrogen atoms of the C₁-C₆ alkyl may be substituted with fluoro. In embodiments, X^{c} is O, NH, N(C₁-C₆ alkyl), N(C)(O)(C₁-C₆ alkyl), N(SO₂—(C₁-C₆ alkyl), or N((C₁-C₆ alkylenyl)O)ₜ(C₁-C₆ alkyl), wherein t is 1, 2, 3, 4 or 5.

In embodiment no. 68, the present invention provides a compound of Formula I as set forth in embodiment no. 67, or a pharmaceutically acceptable salt thereof, wherein X^{c} is NR⁹ and R⁹is (C₁-C₆ alkylenyl)(C₃-C₈ cycloalkyl), C₃-C₈ cycloalkyl, C(O)C₁-C₆ alkyl, —((C₁-C₆ alkylenyl)O)ₜ(C₁-C₆ alkyl), or S(O)₂(C₁-C₆ alkyl) wherein one or more of the hydrogen atoms of the C₁-C₆ alkyl may be substituted with fluoro or hydroxy, and wherein t is 1, 2, 3, 4 or 5.

In embodiment no. 69, the present invention provides a compound of Formula I as set forth in embodiment no. 67, or a pharmaceutically acceptable salt thereof, wherein X^{c} is NR⁹ and R⁹ is —(CH₂)aryl, —(CH₂)(C₃-C₈cycloalkyl), or C₁-C₆ alkyl wherein one or more of the hydrogen atoms of the C₁-C₆ alkyl may be substituted with fluoro.

In embodiment no. 70, the present invention provides a compound of Formula I as set forth in embodiment no. 67, or a pharmaceutically acceptable salt thereof, wherein X^{c} is NR⁹ and R⁹ is H, CH₂CF₃,CH₂CHF₂, CH₂C(CH₃)₂OH, cyclopentyl, phenylmethyl, C(O)CH₃, C(O)C(CH₃)₃, C(O)CHF₂, C(O)CF₂CHF₂, (CH₂CH₂O)₄(CH₃), S(O)₂CH₃, or

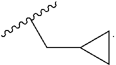 [SMILES: *CC1CC1]

In embodiment no. 71, the present invention provides a compound of Formula I as set forth in any one of embodiment nos. 1-47, or a pharmaceutically acceptable salt thereof, wherein

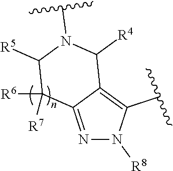

is:

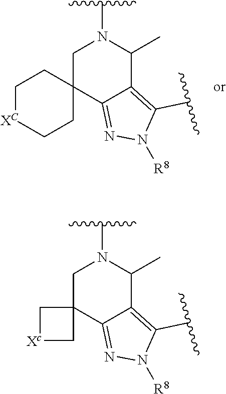

and X^{c} is O, NH, N(C₁-C₆ alkyl), N(C)(O)(C₁-C₆ alkyl), or N(SO₂—(C₁-C₆ alkyl).

In embodiment no. 72, the present invention provides a compound of Formula I as set forth in any one of embodiment nos. 1-71, or a pharmaceutically acceptable salt thereof, wherein

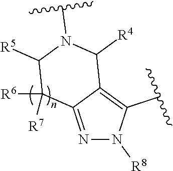

is:

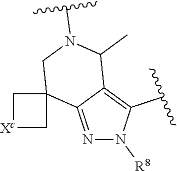

and X^{c} is O, NH, N(C₁-C₆ alkyl), N(C)(O)(C₁-C₆ alkyl), or N(SO₂—(C₁-C₆ alkyl).

In embodiment no. 73, the present invention provides a compound of Formula I as set forth in any one of embodiment nos. 1-71, or a pharmaceutically acceptable salt thereof, wherein

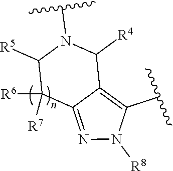

is:

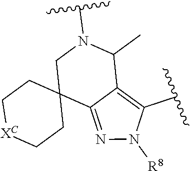

and X^{c} is O, NH, N(C₁-C₆ alkyl), N(C)(O)(C₁-C₆ alkyl), or N(SO₂—(C₁-C₆ alkyl).

In embodiment no. 74, the present invention provides a compound of Formula I as set forth in any one of embodiment nos. 1-47, or a pharmaceutically acceptable salt thereof, wherein

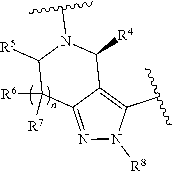

is:

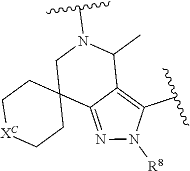

and X^{c} is O, NH, N(C₁-C₆ alkyl), NC(O)(C₁-C₆ alkyl), or NSO₂(C₁-C₆ alkyl).

In embodiment no. 75, the present invention provides a compound of Formula I as set forth in any one of embodiment nos. 1-74, or a pharmaceutically acceptable salt thereof, wherein

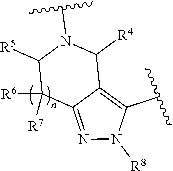

is:

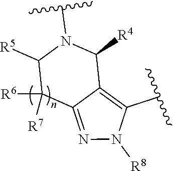

In embodiment no. 76, the present invention provides a compound of Formula I as set forth in any one of embodiment nos. 1-5, 7, 10, 13, 16-17, 19, 22 and 25-75, or a pharmaceutically acceptable salt thereof, wherein X is O.

In embodiment no. 77, the present invention provides a compound of Formula I as set forth in any one of embodiment nos. 1-5, 7, 10, 13, 16-17, 19, 22 and 25-75, or a pharmaceutically acceptable salt thereof, wherein X is =NCH₃ or

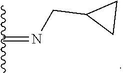 [SMILES: *=NCC1CC1]

In embodiment no. 78, the present invention provides a compound of Formula I as set forth in any one of embodiment nos. 1-77, or a pharmaceutically acceptable salt thereof, wherein R⁴, R⁵, R⁶ and R⁷ are each independently substituted with 0 to 2 substituents independently selected from halogen, NH₂, aminoalkyl, diaminoalkyl, C₁-C₆ alkyl, and C₁-C₆ alkyl substituted with one or more halogen atoms, C₁-C₆ alkoxy, and hydroxy.

In embodiment no. 79, the present invention provides a compound of Formula I as set forth in any one of embodiment nos. 1-6, 16-18 and 25-78, or a pharmaceutically acceptable salt thereof, wherein R^{y}, and R^{z}, together with the atom which they are attached, form a 5-7 membered heterocycloalkyl ring or a 5-6 membered heteroaryl.

In embodiment no. 80, the present invention provides a compound of Formula I as set forth in any one of embodiment nos. 1-6, 16-18 and 25-78, or a pharmaceutically acceptable salt thereof, wherein R^{z} is H.

In embodiment no. 81, the present invention provides a compound of Formula I as set forth in any one of embodiment nos. 1-6, 16-18 and 25-78, or a pharmaceutically acceptable salt thereof, wherein Y is

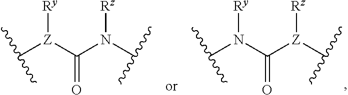

Z is CR^{x}, and R^{x} is H, halo, or CH₃;

In embodiment no. 82, the present invention provides a compound of Formula I as set forth in any one of embodiment nos. 1-6, 16-18 and 25-78, or a pharmaceutically acceptable salt thereof, wherein Y is

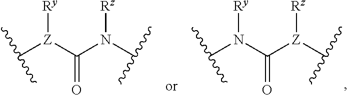

Z is CR^{x} and R^{x} is H and R^{y} is H.

In embodiment no. 83, the present invention provides a compound of Formula I as set forth in any one of embodiment nos. 1-6, 16-18 and 25-78, or a pharmaceutically acceptable salt thereof, wherein Y is

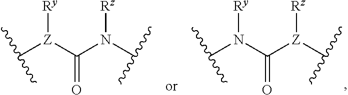

Z is CR^{x} and R^{x} is F and R is F. In an alternative embodiment, R^{x} is H, halo, or C₁-C₆ alkyl. In another alternative embodiment, R^{y}, and R^{z} are each independently H, halo, or C₁-C₆ alkyl.

In embodiment no. 84, the present invention provides a compound of Formula I as set forth in any one of embodiment nos. 1-6, 16-18 and 25-78, or a pharmaceutically acceptable salt thereof, wherein

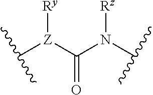

### or Y is

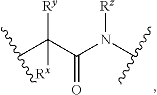

and
R^{x}, R^{y}, and R^{z} are each independently H, halogen, or CH₃. In a class of this embodiment, the halogen is fluoro.

In embodiment no. 85, the present invention provides a compound of Formula I as set forth in any one of embodiment nos. 1-6, 16-18 and 25-78, or a pharmaceutically acceptable salt thereof, wherein Y is

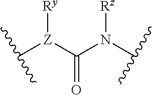

and Z is CR^{x}, and R^{x} and R^{y} together with the carbon to which they are attached, form a spiro cyclopropyl ring and R^{z} is H, fluoro, or methyl.

In embodiment no. 86, the present invention provides a compound of Formula I as set forth in any one of embodiment nos. 1-6, 16-18 and 25-78, or a pharmaceutically acceptable salt thereof, wherein Y is

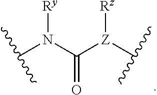

In embodiment no. 87, the present invention provides a compound of Formula I as set forth in any one of embodiment nos. 1-6, 16-18 and 25-78, or a pharmaceutically acceptable salt thereof, wherein Y is

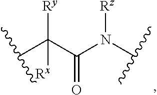

and R^{x}, R^{y}, and R^{z} are each independently H, halogen, or CH₃. In other embodiments, Y is

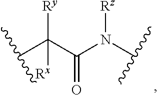

and R^{x}, R^{y}, and R^{z} together with the atom which they are attached, form a 5-8 membered heterocycloalkyl ring, for example

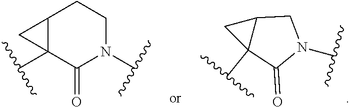 [SMILES: *N1CCC2CC2(*)C1=O ; *N1CC2CC2(*)C1=O]

In embodiment no. 88, the present invention provides a compound of Formula I as set forth in any one of embodiment nos. 1-6, 16-18 and 25-78, or a pharmaceutically acceptable salt thereof, wherein Y is

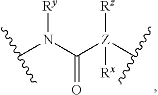

and
R^{x}, R^{y}, and R^{z} are each independently H, halogen, or CH₃.

In embodiment no. 89, the present invention provides a compound of Formula I as set forth in any one of embodiment nos. 1-6, 16-18 and 25-78, or a pharmaceutically acceptable salt thereof, wherein
Y is

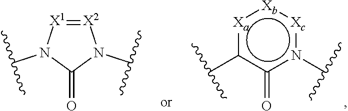

and
X¹ is N or C(H);
X² is N or C(H);
Xₐ is N or C(H);
X_{b} is N or C(H); and
X_{c} is N or C(H).

In embodiment no. 90, the present invention provides a compound of Formula I as set forth in embodiment no. 89, or a pharmaceutically acceptable salt thereof, wherein at least one of X¹ or X² is N.

In embodiment no. 91, the present invention provides a compound of Formula I as set forth in embodiment no. 89, or a pharmaceutically acceptable salt thereof, wherein at least one of Xₐ, X_{b}, and X_{c} is N.

In embodiment no. 92, the present invention provides a compound of Formula I as set forth in any one of embodiment nos. 1-6, 16-18 and 25-78, or a pharmaceutically acceptable salt thereof, wherein
Y is

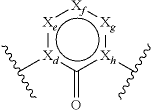

and 1-3 of X_{d}, Xₑ, X_{f}, X_{g}, and Xₕ, X_{d} are nitrogen and the remaining are C(H).

In embodiment no. 93, the present invention provides a compound of Formula I as set forth in embodiment no. 92, or a pharmaceutically acceptable salt thereof, wherein one or two of X_{d}, Xₑ, X_{f}, X_{g}, and Xₕ, are nitrogen and the remaining are C(H).

In embodiment no. 94, the present invention provides a compound of Formula I as set forth in any one of embodiment nos. 1-6, 16-18 and 25-78, or a pharmaceutically acceptable salt thereof, wherein
Y is

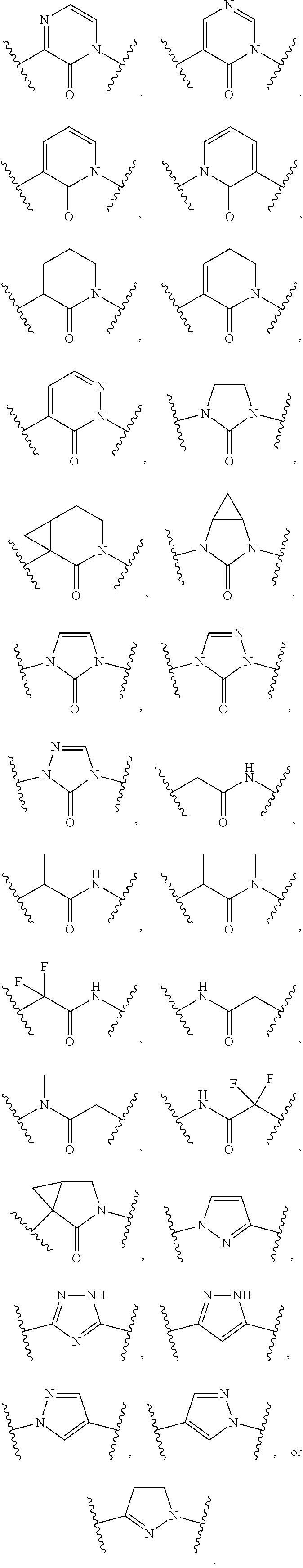

In embodiment no. 95, the present invention provides a compound of Formula I as set forth in any one of embodiment nos. 1-6, 16-18 and 25-78, or a pharmaceutically acceptable salt thereof, wherein
Y is

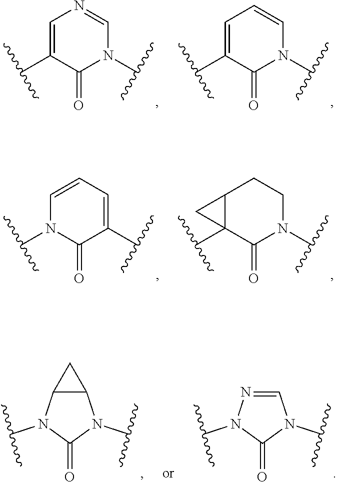 [SMILES: *c1cncn(*)c1=O ; *c1cccn(*)c1=O ; *c1cccn(*)c1=O ; *N1CCC2CC2(*)C1=O ; *N1C(=O)N(*)C2CC21 ; *n1cnn(*)c1=O]

In embodiment no. 96 the present invention provides a compound of Formula I as set forth in any one of embodiment nos. 1-6, 16-18 and 25-78, or a pharmaceutically acceptable salt thereof, wherein
Y is

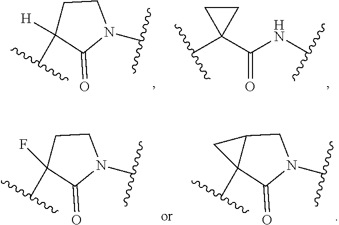 [SMILES: *C1CCN(*)C1=O ; *NC(=O)C1(*)CC1 ; *N1CCC(*)(F)C1=O ; *N1CC2CC2(*)C1=O]

In embodiment no. 97, the present invention provides a compound of Formula I as set forth in any one of embodiment nos. 1-6, 16-18 and 25-78, or a pharmaceutically acceptable salt thereof, wherein Y is:

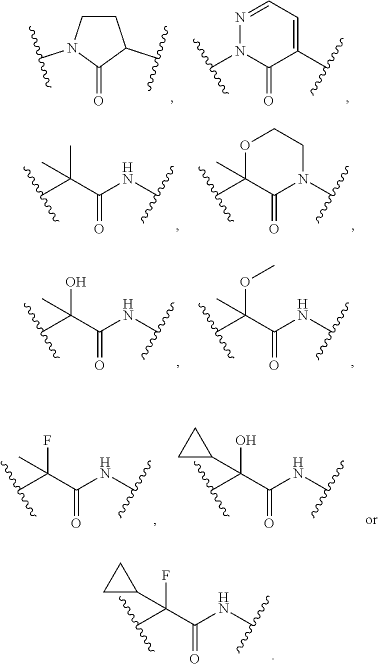

In a sub-embodiment of embodiment no. 97, Y is

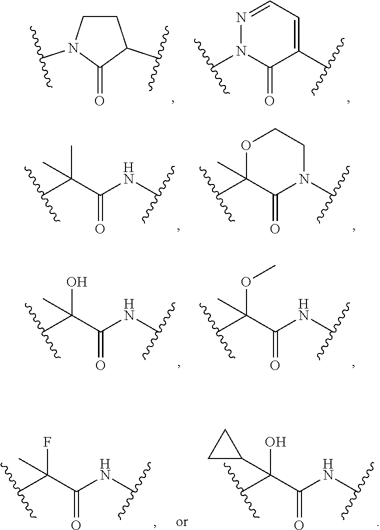

In embodiment no. 98, the present invention provides a compound of Formula I as set forth in any one of embodiment nos. 1-6, 16-18 and 25-78, or a pharmaceutically acceptable salt thereof, wherein Y is:

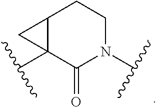 [SMILES: *N1CCC2CC2(*)C1=O]

In embodiment no. 99, the present invention provides a compound of Formula I as set forth in any one of embodiment nos. 1-6, 16-18 and 25-78, or a pharmaceutically acceptable salt thereof, wherein Y is:

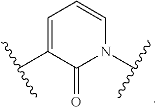 [SMILES: *c1cccn(*)c1=O]

In embodiment no. 100, the present invention provides a compound of Formula I as set forth in any one of embodiment nos. 1-6, 16-18 and 25-78, or a pharmaceutically acceptable salt thereof, wherein Y is:

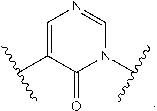 [SMILES: *c1cncn(*)c1=O]

In embodiment no. 101, the present invention provides a compound of Formula I as set forth in any one of embodiment nos. 1-6, 16-18 and 25-78, or a pharmaceutically acceptable salt thereof, wherein Y is:

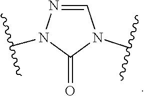 [SMILES: *n1cnn(*)c1=O]

In embodiment no. 102, the present invention provides a compound of Formula I as set forth in any one of embodiment nos. 1-6, 16-18 and 25-78, or a pharmaceutically acceptable salt thereof, wherein Y is:

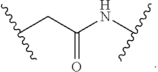 [SMILES: *CC(=O)N*]

In embodiment no. 103, the present invention provides a compound of Formula I as set forth in any one of embodiment nos. 1-6, 16-18 and 25-78, or a pharmaceutically acceptable salt thereof, wherein Y is:

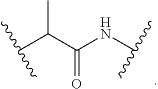 [SMILES: *NC(=O)C(*)C]

In embodiment no. 104, the present invention provides a compound of Formula I as set forth in any one of embodiment nos. 1-6, 16-18 and 25-78, or a pharmaceutically acceptable salt thereof, wherein Y is:

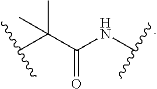 [SMILES: *NC(=O)C(*)(C)C]

In embodiment no. 105, the present invention provides a compound of Formula I as set forth in any one of embodiment nos. 1-6, 16-18 and 25-78, or a pharmaceutically acceptable salt thereof, wherein Y is:

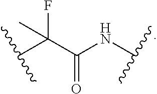 [SMILES: *NC(=O)C(*)(C)F]

In embodiment no. 106, the present invention provides a compound of Formula I as set forth in any one of embodiment nos. 1-78, or a pharmaceutically acceptable salt thereof, wherein Y is:

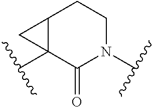 [SMILES: *N1CCC2CC2(*)C1=O]

### and B is

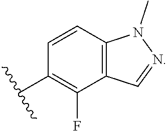 [SMILES: *c1ccc2c(cnn2C)c1F]

In embodiment no. 107, the present invention provides a compound of Formula I as set forth in any one of embodiment nos. 1-78, or a pharmaceutically acceptable salt thereof, wherein Y is:

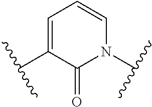 [SMILES: *c1cccn(*)c1=O]

### and B is

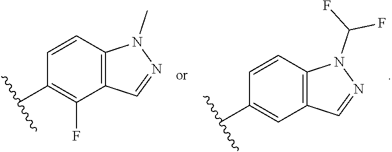 [SMILES: *c1ccc2c(cnn2C)c1F ; *c1ccc2c(cnn2C(F)F)c1]

In embodiment no. 108, the present invention provides a compound of Formula I as set forth in any one of embodiment nos. 1-78, or a pharmaceutically acceptable salt thereof, wherein Y is:

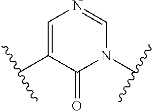 [SMILES: *c1cncn(*)c1=O]

### and B is

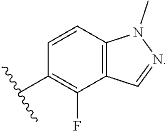 [SMILES: *c1ccc2c(cnn2C)c1F]

In embodiment no. 109, the present invention provides a compound of Formula I as set forth in any one of embodiment nos. 1-78, or a pharmaceutically acceptable salt thereof, wherein Y is:

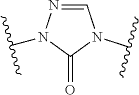 [SMILES: *n1cnn(*)c1=O]

### and B is

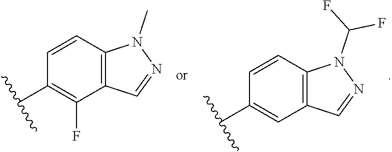 [SMILES: *c1ccc2c(cnn2C)c1F ; *c1ccc2c(cnn2C(F)F)c1]

In embodiment no. 110, the present invention provides a compound of Formula I as set forth in any one of embodiment nos. 1-78, or a pharmaceutically acceptable salt thereof, wherein Y is

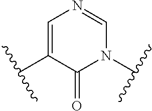 [SMILES: *c1cncn(*)c1=O]

and B is other than

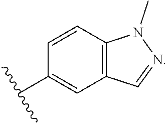 [SMILES: *c1ccc2c(cnn2C)c1]

In embodiment no. 111, the present invention provides a compound of Formula I as set forth in any one of embodiment nos. 98-110, or a pharmaceutically acceptable salt thereof, wherein A is

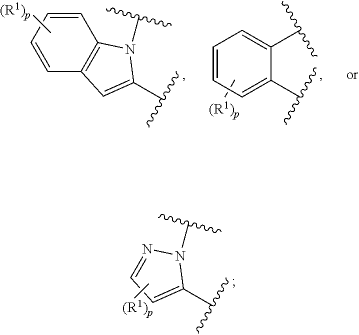

R¹ is

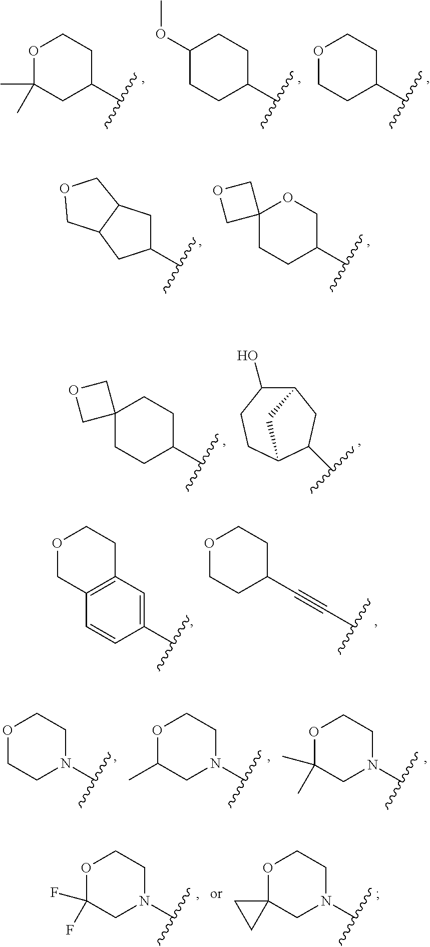

R² is H;
R³ is H or methyl;
R⁴ is hydrogen, methyl, or ethyl;
R⁵ is hydrogen or methyl;
   or R⁴ and R⁵, together with the atom which they are attached, or R⁴ and R⁶, together with the atom which they are attached, form a bridged C₇₋₈ heterocyclic ring fused to the pyrazole; and
R⁶ is H, halo, or methyl, and R⁷ is H, halo, or methyl, or
R⁶ and R⁷ together with the atom to which they are attached, form a cyclopropyl ring or a cyclobutyl ring provided that if there are two R⁶ and R⁷ then only one set forms a ring and the other R⁶ and R⁷ is each independently H or methyl.

In embodiment no. 112, the present invention provides a compound of Formula I as set forth in embodiment no. 111, or a pharmaceutically acceptable salt thereof, wherein B is

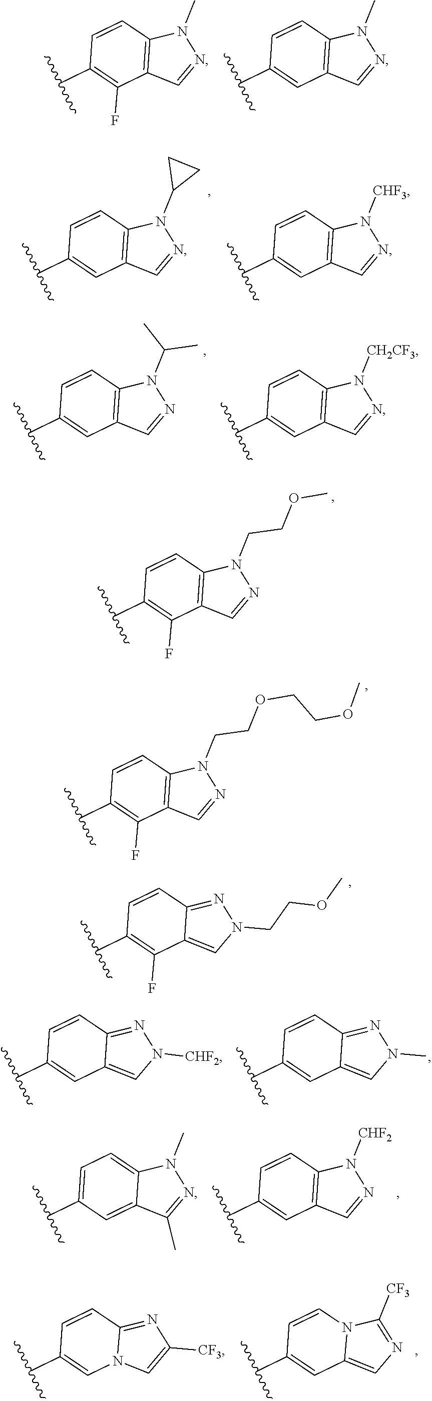

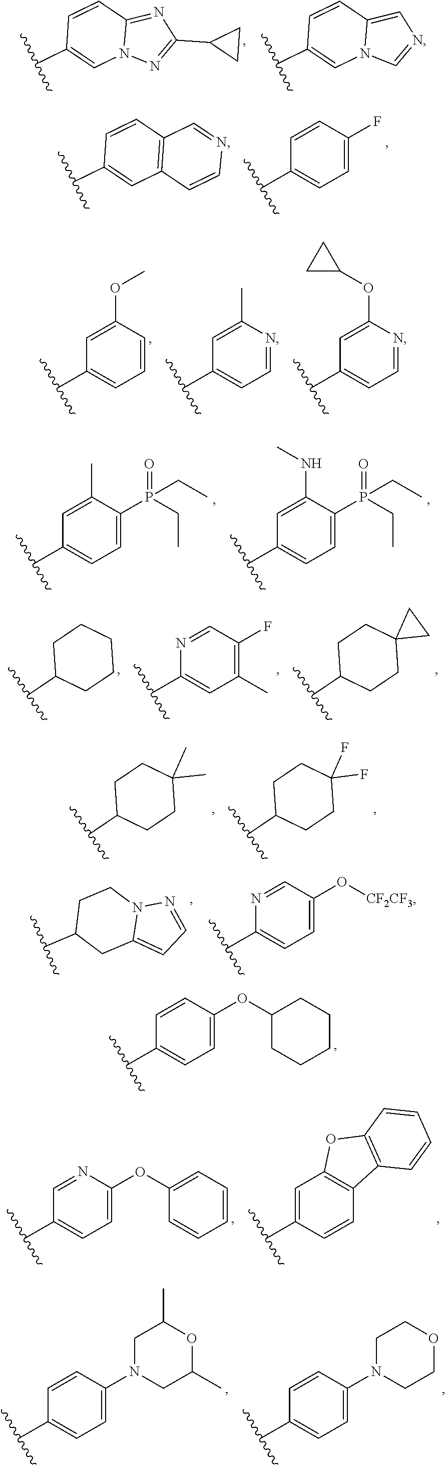

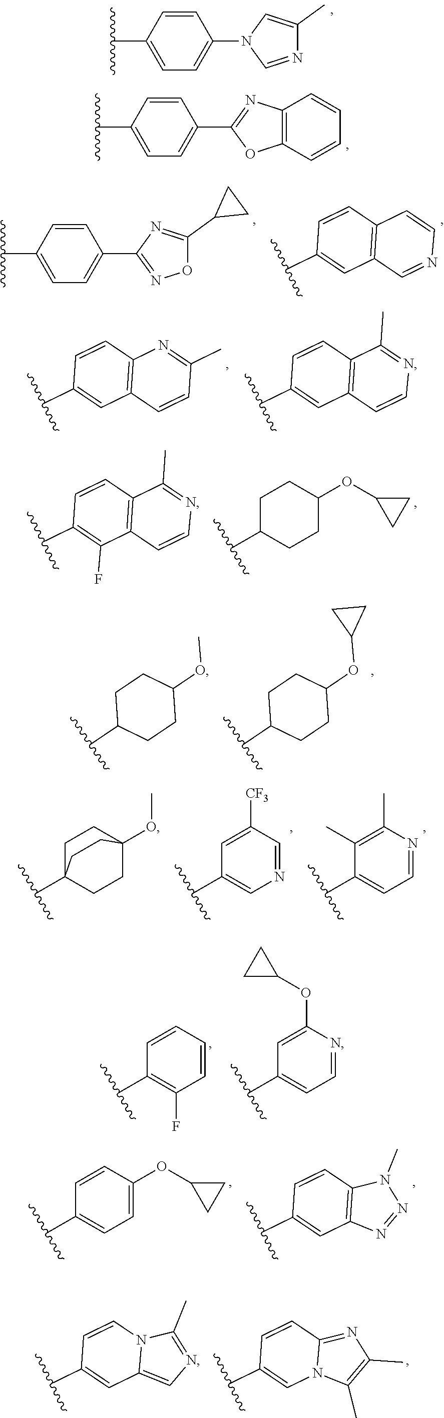

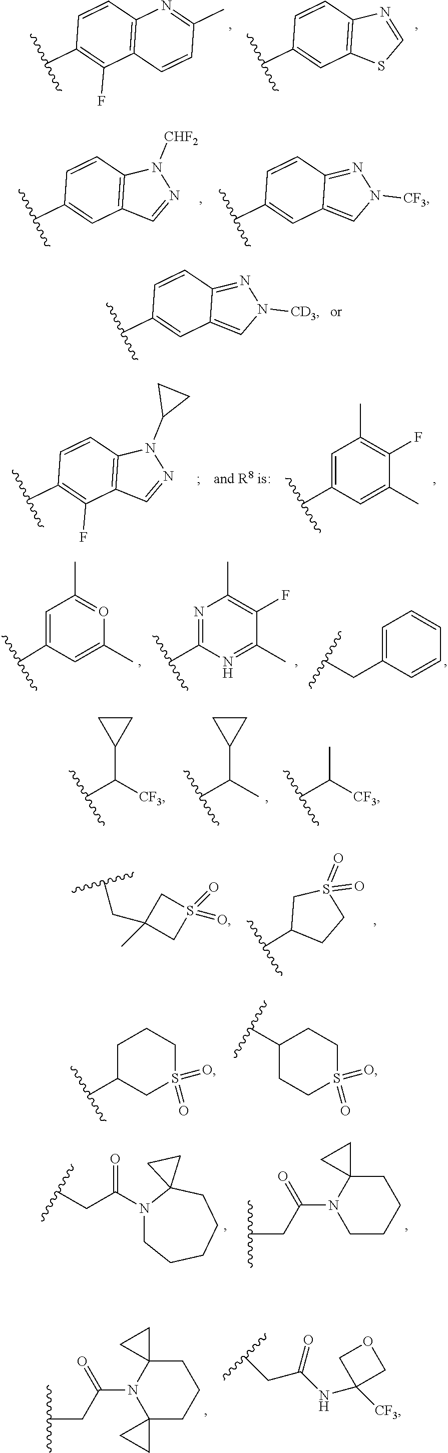

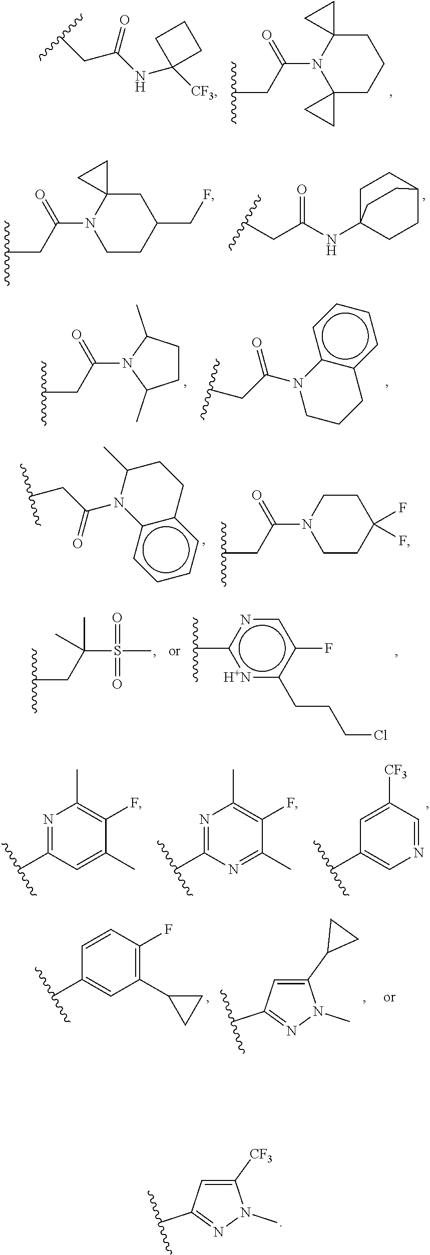

In embodiment no. 113, the present invention provides a compound of Formula I as set forth in embodiment no. 114, or a pharmaceutically acceptable salt thereof, wherein
R⁸ is:

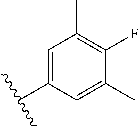 [SMILES: *c1cc(C)c(F)c(C)c1]

### and B is

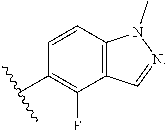 [SMILES: *c1ccc2c(cnn2C)c1F]

In embodiment no. 114, the present invention provides a compound of Formula I as set forth in any one of embodiment nos. 1-105, or a pharmaceutically acceptable salt thereof, wherein B is 5-13 membered heterocycloalkyl, aryl or heteroaryl, and B is substituted by 0, 1 or 2 substituents, each independently selected from C₁-C₃ alkyl, halo, —C₁-C₆ alkylenyl-O—(C₁-C₃ alkyl), C₁-C₅ alkylenyl-O— C₁-C₃ alkylenyl-O— C₁-C₃ alkyl, —O(C₁-C₃ alkyl), —NH(C₁-C₃ alkyl), —O(C₃-C₆ cycloalkyl), 5-9 membered heterocycloalkyl, —O(C₆-C₁₀ aryl), and phosphorus atom substituted with oxo and/or C₁-C₃ alkyl, wherein one or more of the hydrogen atoms of the —C₁-C₃ alkyl or —O(C₁-C₃ alkyl) may be substituted with fluoro, and wherein the 5-9 membered heterocycloalkyl may be unsubstituted or substituted with 1, 2, or 3 substituents independently selected from —C₁-C₃ alkyl, —C₁-C₃ haloalkyl, and oxo.

In embodiment no. 115, the present invention provides a compound of Formula I as set forth in any one of embodiment nos. 1-103 and 106, or a pharmaceutically acceptable salt thereof, wherein B is substituted by 1 or 2 substituents, each independently selected from fluoro, methyl, CHF₂, CF₃, cyclopropyl, OCF₂CF₃,

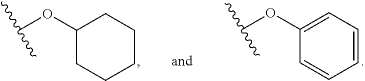 [SMILES: *OC1CCCCC1 ; *Oc1ccccc1]

In embodiment no. 116, the present invention provides a compound of Formula I as set forth in any one of embodiment nos. 1-105, 110 and 114-115, or a pharmaceutically acceptable salt thereof, wherein
B is

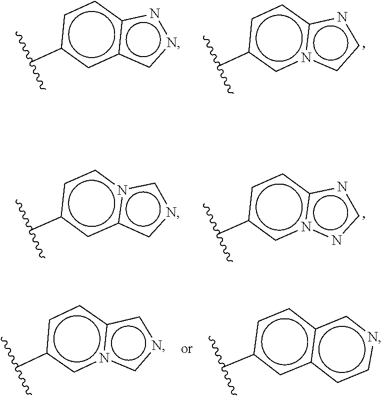

and B is substituted by 0, 1 or 2 substituents, as provided for in embodiments 1-105, 110 or 114-115.

In embodiment no. 117, the present invention provides a compound of Formula I as set forth in any one of embodiment nos. 1-105, 110 or 114-115, or a pharmaceutically acceptable salt thereof, wherein B is substituted by 1 or 2 C₁-C₃ alkyl.

In embodiment no. 118, the present invention provides a compound of Formula I as set forth in any one of embodiment nos. 1-105, or a pharmaceutically acceptable salt thereof, wherein
B is

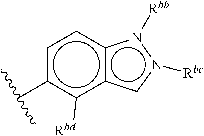

and
one of R^{bb} and R^{bc} is C₁-C₃ alkyl or heteroalkyl, and the other is not present,
R^{bd} is hydrogen or halogen,
   wherein one or more of the hydrogen atoms of the —C₁-C₃ alkyl or heteroalkyl may be substituted with fluoro.

In embodiment no. 119, the present invention provides a compound of Formula I as set forth in any one of embodiment nos. 1-105 and 111, or a pharmaceutically acceptable salt thereof, wherein
B is

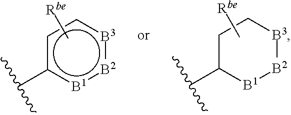

wherein at least two of B¹, B² and B³ are CH or CH₂, and the third is CH, CH₂, N or NH, and R^{be} represents one, or two optional ring substituents, which can be the same or different at each occurrence and wherein each occurrence of R^{e} is independently selected from C₁-C₃ alkyl, halo, —O(C₁-C₃ alkyl), —O(C₃-C₆ cycloalkyl), 5-9 membered heterocycloalkyl, —O(C₆-C₁₀ aryl) and phosphorus atom substituted with oxo and/or C₁-C₃ alkyl, wherein one or more of the hydrogen atoms of the —C₁-C₃ alkyl or —O(C₁-C₃ alkyl) is optionally substituted with fluoro, and wherein the 5-9 membered heterocycloalkyl may be unsubstituted or substituted with 1, 2, or 3 substituents independently selected from —C₁-C₃ alkyl, —C₁-C₃ haloalkyl, —C₃-C₆ cycloalkyl, and oxo.

In embodiment no. 120, the present invention provides a compound of Formula I as set forth in embodiment no. 118, or a pharmaceutically acceptable salt thereof, wherein the heteroalkyl is —O(C₁-C₃ alkyl).

In embodiment no. 121, the present invention provides a compound of Formula I as set forth in any one of embodiment nos. 1-105, 111 and 119-120, or a pharmaceutically acceptable salt thereof, wherein
B is substituted with C₁-C₃ fluoroalkyl.

In embodiment no. 122, the present invention provides a compound of Formula I as set forth in any one of embodiment nos. 1-105, 111 and 119-120, or a pharmaceutically acceptable salt thereof, wherein
B is substituted by 1 or 2 substituents, each independently selected from fluoro, methyl, —CHF₂, CF₃, —CH₂CF₃, —OCF₂CF₃, —CH₂CH₂—O—CH₂CH₂—O—CH₃, —CH₂CH₂—O—CH₃, —OCH₃, NH(CH₃), morpholinyl, 3-methyl-2-oxoimidazolidinyl,

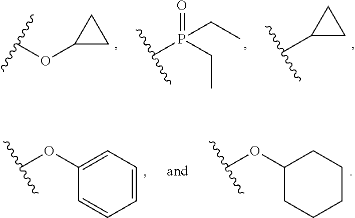

In embodiment no. 123, the present invention provides a compound of Formula I as set forth in any one of embodiment nos. 105, 111 and 119-122, or a pharmaceutically acceptable salt thereof, wherein
B is substituted by 1 or 2 substituents, each independently selected from fluoro, methyl, —CHF₂, CF₃, —CH₂CF₃, —OCF₂CF₃, —CH₂CH₂—O—CH₂CH₂—O—CH₃, —CH₂CH₂—O—CH₃, —OCH₃, NH(CH₃), 3-methyl-2-oxoimidazolidinyl,

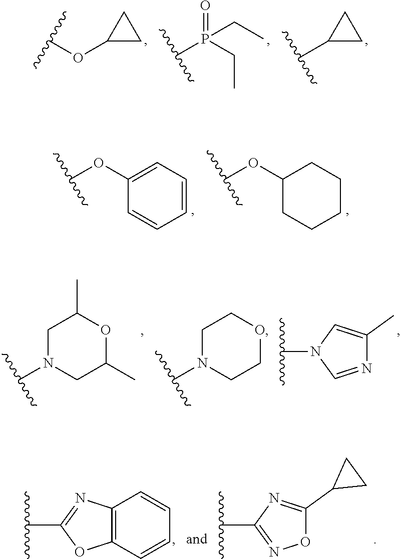

In embodiment no. 124, the present invention provides a compound of Formula I as set forth in any one of embodiment nos. 1-105 and 111, or a pharmaceutically acceptable salt thereof, wherein B is

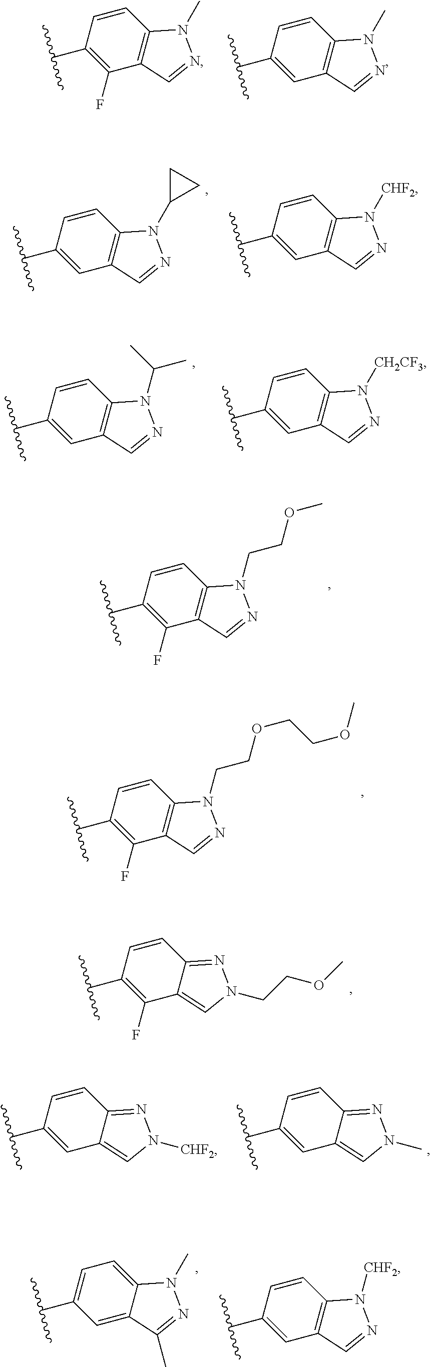

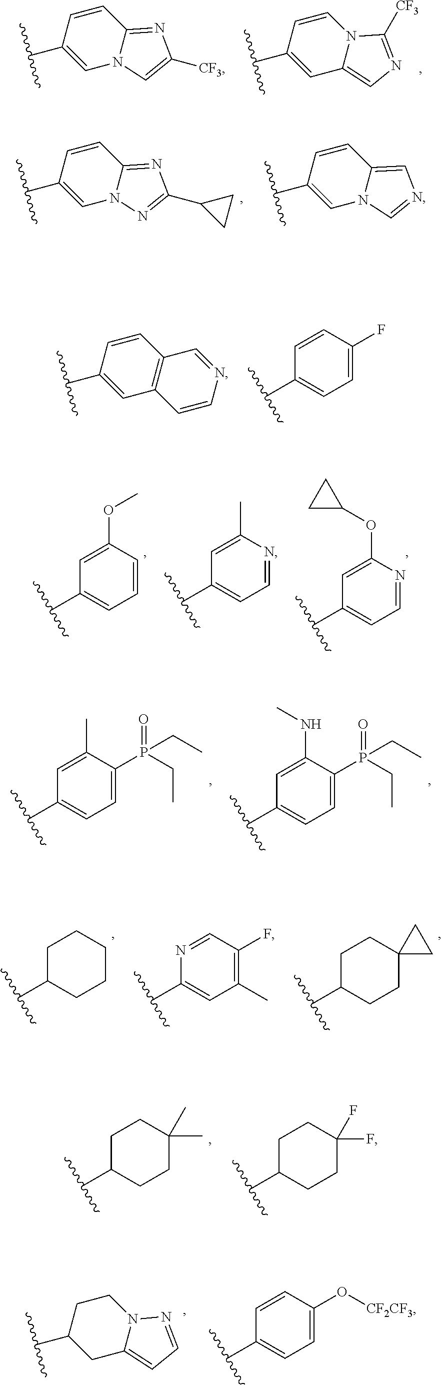

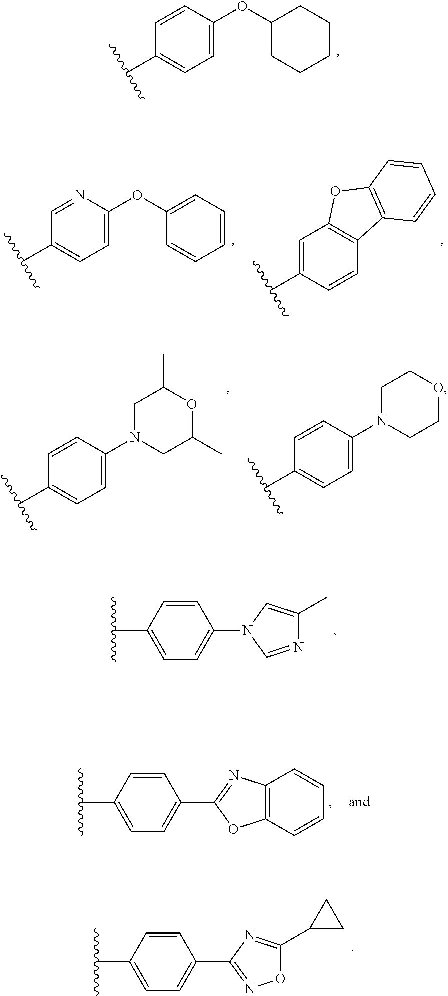

In embodiment no. 125, the present invention provides a compound of Formula I as set forth in any one of embodiment nos. 1-105 and 111, or a pharmaceutically acceptable salt thereof, wherein B is

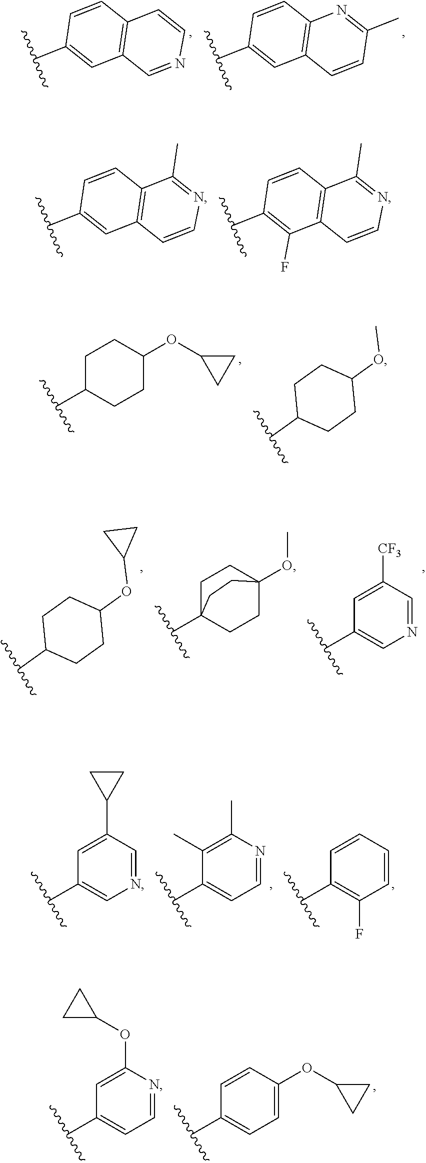

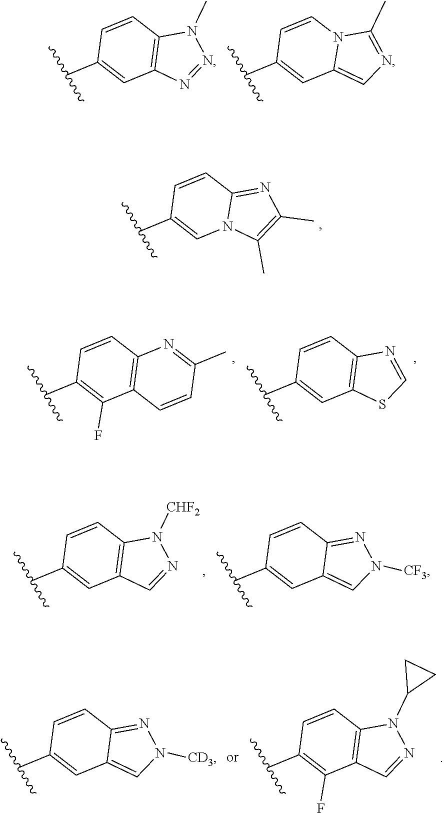

In embodiment no. 126, the present invention provides a compound of Formula I as set forth in any one of embodiment nos. 1-105 and 111, or a pharmaceutically acceptable salt thereof, wherein B is

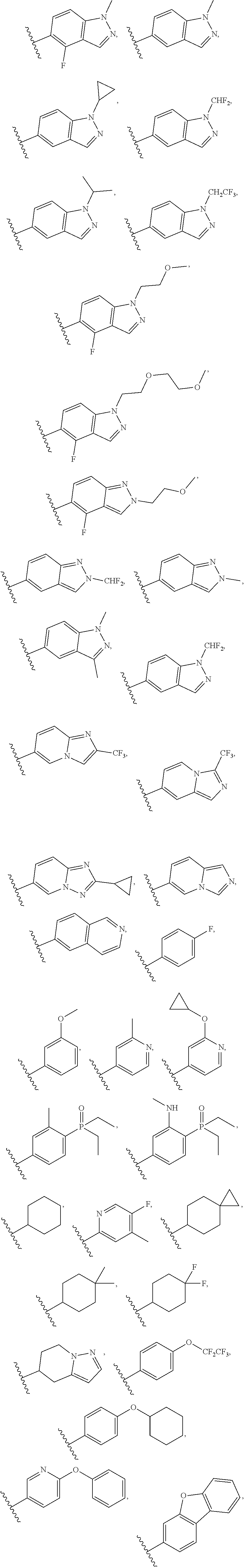

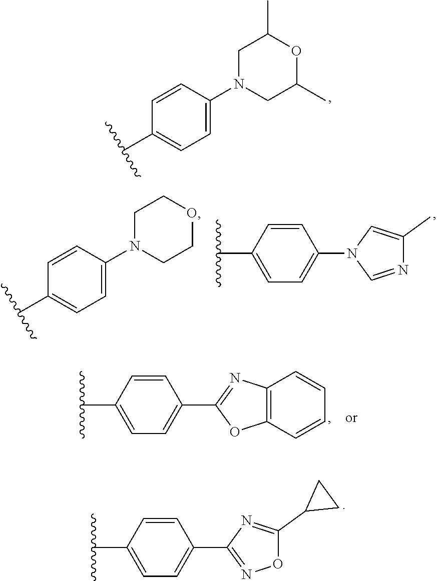

In embodiment no. 127, the present invention provides a compound of Formula I as set forth in any one of embodiment nos. 1-105 and 111, or a pharmaceutically acceptable salt thereof, wherein B is

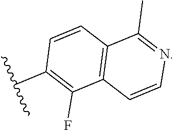 [SMILES: *c1ccc2c(C)nccc2c1F]

In embodiment no. 128, the present invention provides a compound of Formula I as set forth in any one of embodiment nos. 1-111 and 119-120, or a pharmaceutically acceptable salt thereof, wherein R⁸ is substituted by 1 or 2 substituents, each independently selected from fluoro, methyl, CF₃, CHF₂, cyclopropyl, cyclopropyloxy, and oxo. In embodiment no. 129, R⁸ is substituted by 1 or 2 substituents, each independently selected from fluoro, methyl, CF₃, cyclopropyl, and oxo.

In embodiment no. 130, the present invention provides a compound of Formula I as set forth in any one of embodiment nos. 1-111 and 119-120, or a pharmaceutically acceptable salt thereof, wherein R⁸ is

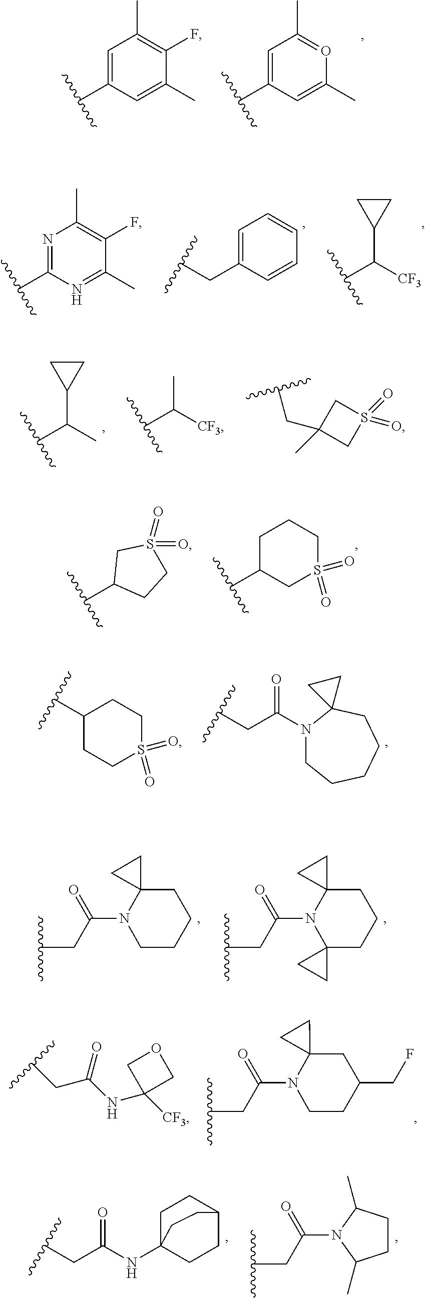

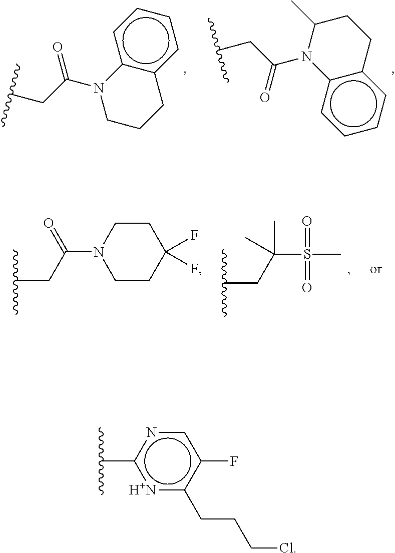

In a sub-embodiment of embodiment no. 130, wherein R⁸ is:

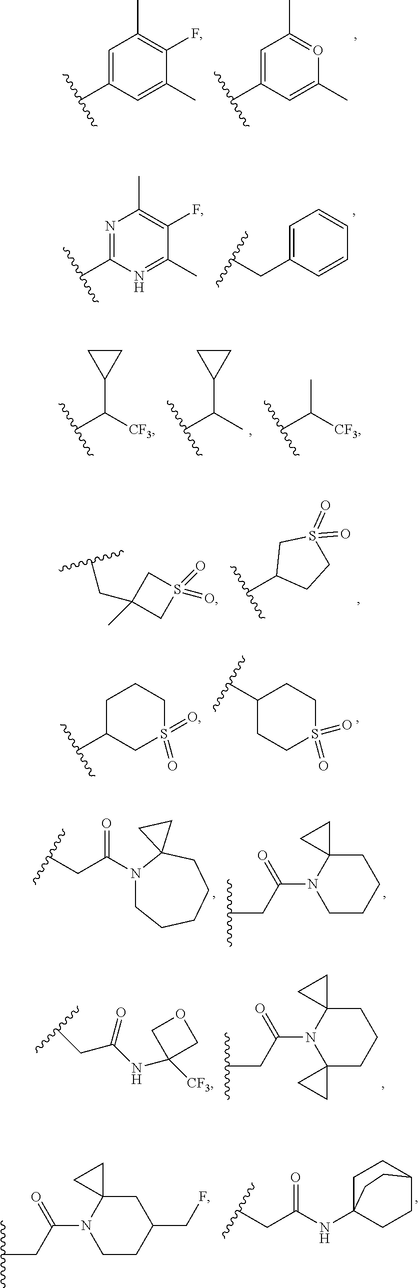

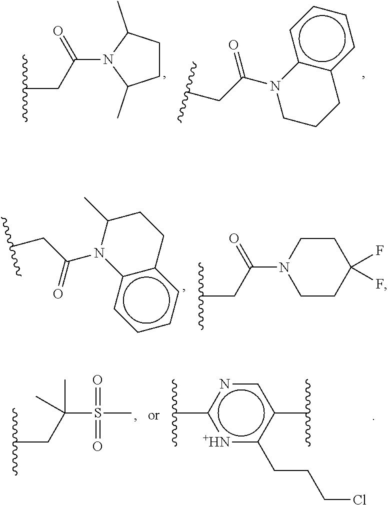

In embodiment no. 131, the present invention provides a compound of Formula I as set forth in any one of embodiment nos. 1-111 and 119-120, or a pharmaceutically acceptable salt thereof, wherein R⁸ is:

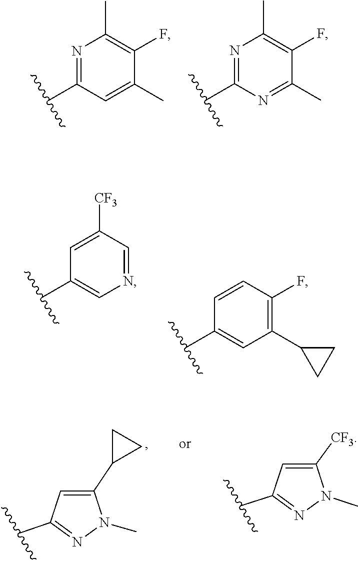

In embodiment no. 132, the present invention provides a compound of Formula I as set forth in any one of embodiment nos. 1-111 and 119-120, or a pharmaceutically acceptable salt thereof, wherein R⁸ is:

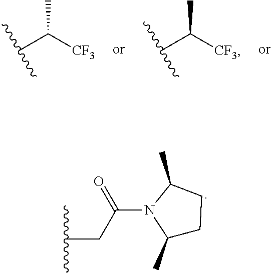

In embodiment no. 132, the present invention provides a compound of Formula I as set forth in any one of embodiment nos. 1-133, or a pharmaceutically acceptable salt thereof, wherein P is 1.

In embodiment no. 133, the present invention provides a compound selected from:
3-((1S,2S)-1-(2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4,7,7-trimethyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3′-(3-(4-fluoro-1-methyl-1H-indazol-5-yl)-2-oxo-2,3-dihydro-1H-imidazol-1-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-2,4,5,6,7,8-hexahydropyrazolo[4,3-c]azepine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-2,4,5,6,7,8-hexahydropyrazolo[4,3-c]azepine-5-carbonyl)-1H-indol-1-yl)-2-methylcyclopropyl)-1,2,4-oxadiazol-5(4H)-one;
ethyl 2-((S)-3-(3-(4-fluoro-1-methyl-1H-indazol-5-yl)-2-oxo-2,3-dihydro-1H-imidazol-1-yl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-2-yl)acetate;
(S)-3-(1-(5-bromo-2-(3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)cyclopropyl)-1,2,4-oxadiazol-5(4H)-one;
3-((1S,2S)-1-(2-((S)-2-benzyl-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-((S)-1-cyclopropylethyl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-((R)-1-cyclopropylethyl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-3-(3-(4-fluoro-1-methyl-1H-indazol-5-yl)-2-oxo-2,3-dihydro-1H-imidazol-1-yl)-4-methyl-2-(2-methyl-2-(methylsulfonyl)propyl)-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(5-(2,2-dimethyltetrahydro-2H-pyran-4-yl)-2-((S)-2-(1,1-dioxidotetrahydro-2H-thiopyran-4-yl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(2-((S)-3-(3-(4-fluoro-1-methyl-1H-indazol-5-yl)-2-oxo-2,3-dihydro-1H-imidazol-1-yl)-4-methyl-2-((3-methyl-1,1-dioxidothietan-3-yl)methyl)-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-3′-(3-(4-fluoro-1-methyl-1H-indazol-5-yl)-2-oxo-2,3-dihydro-1H-imidazol-1-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4,7,7-trimethyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
(4′S)-5′-(5-(2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-3′-(3-(4-fluoro-1-methyl-1H-indazol-5-yl)-2-oxo-2,3-dihydro-1H-imidazol-1-yl)-2′-(4-fluoro-3,5-dimethylphenyl)-4′-methyl-2′,4′,5′,6′-tetrahydrospiro[cyclobutane-1,7′-pyrazolo[4,3-c]pyridin]-1′-ium;
3-((1S,2S)-1-(2-((S)-3′-(3-(4-fluoro-1-methyl-1H-indazol-5-yl)-2-oxo-2,3-dihydro-1H-imidazol-1-yl)-2′-(4-fluoro-3,5-dimethylphenyl)-4′-methyl-2′,4′,5′,6′-tetrahydrospiro[cyclobutane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(5-(2,2-dimethyltetrahydro-2H-pyran-4-yl)-2-((S)-3′-(3-(4-fluoro-1-(2-methoxyethyl)-1H-indazol-5-yl)-2-oxo-2,3-dihydro-1H-imidazol-1-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-1H-indol-1-yl)-2-methylcyclopropyl)-1,2,4-oxadiazol-5(4H)-one;
3-((1S,2S)-1-(5-(2,2-dimethyltetrahydro-2H-pyran-4-yl)-2-((S)-3′-(3-(4-fluoro-2-(2-methoxyethyl)-2H-indazol-5-yl)-2-oxo-2,3-dihydro-1H-imidazol-1-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-1H-indol-1-yl)-2-methylcyclopropyl)-1,2,4-oxadiazol-5(4H)-one;
3-((1S,2S)-1-(5-(2,2-dimethyltetrahydro-2H-pyran-4-yl)-2-((S)-3′-(3-(4-fluoro-1-(2-(2-methoxyethoxy)ethyl)-1H-indazol-5-yl)-2-oxo-2,3-dihydro-1H-imidazol-1-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-1H-indol-1-yl)-2-methylcyclopropyl)-1,2,4-oxadiazol-5(4H)-one;
3-((1S,2S)-1-(2-((S)-3′-(3-(4-fluoro-1-methyl-1H-indazol-5-yl)-2-oxo-2,3-dihydro-1H-imidazol-1-yl)-2′-(4-fluoro-3,5-dimethylphenyl)-4′-methyl-2,2′,3,4′,5,5′,6,6′-octahydrospiro[pyran-4,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-((S)-1-cyclopropyl-2,2,2-trifluoroethyl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-((R)-1-cyclopropyl-2,2,2-trifluoroethyl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-((R)-1-cyclopropylethyl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-((S)-2,2-dimethyltetrahydro-2H-pyran-4-yl)-1H-indol-1-yl)-2-methylcyclopropyl)-1,2,4-oxadiazol-5(4H)-one or 3-((1S,2S)-1-(2-((S)-2-((S)-1-cyclopropylethyl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-((S)-2,2-dimethyltetrahydro-2H-pyran-4-yl)-1H-indol-1-yl)-2-methylcyclopropyl)-1,2,4-oxadiazol-5(4H)-one;
3-((1S,2S)-1-(2-((S)-2-((R)-1-cyclopropylethyl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-((S)-2,2-dimethyltetrahydro-2H-pyran-4-yl)-1H-indol-1-yl)-2-methylcyclopropyl)-1,2,4-oxadiazol-5(4H)-one;
3-((1S,2S)-1-(2-((S)-2-((S)-1-cyclopropylethyl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-((S)-2,2-dimethyltetrahydro-2H-pyran-4-yl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-2-((S)-1,1-dioxidotetrahydro-2H-thiopyran-3-yl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-2-((R)-1,1-dioxidotetrahydro-2H-thiopyran-3-yl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-2-((S)-1,1-dioxidotetrahydro-2H-thiopyran-3-yl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-2-((R)-1,1-dioxidotetrahydro-2H-thiopyran-3-yl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(2-((S)-2-((S)-1,1-dioxidotetrahydrothiophen-3-yl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-((R)-1,1-dioxidotetrahydrothiophen-3-yl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-((S)-1,1-dioxidotetrahydrothiophen-3-yl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one or 3-((1S,2S)-1-(2-((S)-2-((R)-1,1-dioxidotetrahydrothiophen-3-yl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((4S,7R)-3-(3-(4-fluoro-1-methyl-1H-indazol-5-yl)-2-oxo-2,3-dihydro-1H-imidazol-1-yl)-2-(4-fluoro-3,5-dimethylphenyl)-2,4,5,6,7,8-hexahydro-4,7-epiminocyclohepta[c]pyrazole-9-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((4R,7S)-3-(3-(4-fluoro-1-methyl-1H-indazol-5-yl)-2-oxo-2,3-dihydro-1H-imidazol-1-yl)-2-(4-fluoro-3,5-dimethylphenyl)-2,4,5,6,7,8-hexahydro-4,7-epiminocyclohepta[c]pyrazole-9-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((4S,7R)-3-(3-(4-fluoro-1-methyl-1H-indazol-5-yl)-2-oxo-2,3-dihydro-1H-imidazol-1-yl)-2-(4-fluoro-3,5-dimethylphenyl)-2,4,5,6,7,8-hexahydro-4,7-epiminocyclohepta[c]pyrazole-9-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((4R,7S)-3-(3-(4-fluoro-1-methyl-1H-indazol-5-yl)-2-oxo-2,3-dihydro-1H-imidazol-1-yl)-2-(4-fluoro-3,5-dimethylphenyl)-2,4,5,6,7,8-hexahydro-4,7-epiminocyclohepta[c]pyrazole-9-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-((2R,4r,6S)-2,6-dimethyltetrahydro-2H-pyran-4-yl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-((2R,4s,6S)-2,6-dimethyltetrahydro-2H-pyran-4-yl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-((2R,4r,6S)-2,6-dimethyltetrahydro-2H-pyran-4-yl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-((2R,4s,6S)-2,6-dimethyltetrahydro-2H-pyran-4-yl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-(1-(4-fluoro-1-methyl-1H-indazol-5-yl)-2-oxo-1,2-dihydropyridin-3-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-3-(1-(4-fluoro-1-methyl-1H-indazol-5-yl)-2-oxo-1,2-dihydropyridin-3-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-2-(4-fluoro-3,5-dimethylphenyl)-4-methyl-3-(1-(1-methyl-1H-indazol-5-yl)-2-oxo-1,2-dihydropyridin-3-yl)-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-2-(4-fluoro-3,5-dimethylphenyl)-4-methyl-3-(1-(1-methyl-1H-indazol-5-yl)-6-oxo-1,6-dihydropyrimidin-5-yl)-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(2-((S)-3-(1-(1-(difluoromethyl)-1H-indazol-5-yl)-2-oxo-1,2-dihydropyridin-3-yl)-2-(4-fluoro-3,5-dimethylphenyl)-4,7,7-trimethyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-(1-(2-(difluoromethyl)-2H-indazol-5-yl)-2-oxo-1,2-dihydropyridin-3-yl)-2-(4-fluoro-3,5-dimethylphenyl)-4,7,7-trimethyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-(4-fluoro-3,5-dimethylphenyl)-4-methyl-3-(1-(1-methyl-1H-indazol-5-yl)-2-oxo-1,2-dihydropyridin-3-yl)-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-(4-fluoro-3,5-dimethylphenyl)-4,7,7-trimethyl-3-(1-(2-methyl-2H-indazol-5-yl)-2-oxo-1,2-dihydropyridin-3-yl)-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-(1-(1,3-dimethyl-1H-indazol-5-yl)-2-oxo-1,2-dihydropyridin-3-yl)-2-(4-fluoro-3,5-dimethylphenyl)-4,7,7-trimethyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-(4-fluoro-3,5-dimethylphenyl)-4,7,7-trimethyl-3-(1-(1-methyl-1H-indazol-5-yl)-2-oxo-1,2-dihydropyridin-3-yl)-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3′-(1-(1,3-dimethyl-1H-indazol-5-yl)-2-oxo-1,2-dihydropyridin-3-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2′-(4-fluoro-3,5-dimethylphenyl)-4′-methyl-3′-(1-(2-methyl-2H-indazol-5-yl)-2-oxo-1,2-dihydropyridin-3-yl)-2′,4′,5′,6′-tetrahydrospiro[cyclopropane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-(1-(4-fluoro-1-methyl-1H-indazol-5-yl)-2-oxo-1,2-dihydropyridin-3-yl)-2-(4-fluoro-3,5-dimethylphenyl)-4,7,7-trimethyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-3-(1-(4-fluoro-1-methyl-1H-indazol-5-yl)-2-oxo-1,2-dihydropyridin-3-yl)-2-(4-fluoro-3,5-dimethylphenyl)-4,7,7-trimethyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(2-((S)-3-(1-(2-(difluoromethyl)-2H-indazol-5-yl)-2-oxo-1,2-dihydropyridin-3-yl)-2-(4-fluoro-3,5-dimethylphenyl)-4,7,7-trimethyl-4,5,6,7-tetrahydro-2H-pyrazolo[4,3-c]pyridine-5-carbonyl)-5-((S)-2,2-dimethyltetrahydro-2H-pyran-4-yl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-2-(4-fluoro-3,5-dimethylphenyl)-4,7,7-trimethyl-3-(1-(1-methyl-1H-indazol-5-yl)-2-oxo-1,2-dihydropyridin-3-yl)-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(2-((S)-2′-(4-fluoro-3,5-dimethylphenyl)-4′-methyl-3′-(1-(1-methyl-1H-indazol-5-yl)-2-oxo-1,2-dihydropyridin-3-yl)-2′,4′,5′,6′-tetrahydrospiro[cyclopropane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-(4-fluoro-3,5-dimethylphenyl)-4-methyl-3-(2-oxo-1-(2-(trifluoromethyl)imidazo[1,2-a]pyridin-6-yl)-1,2-dihydropyridin-3-yl)-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-(1-(2-cyclopropyl-[1,2,4]triazolo[1,5-a]pyridin-6-yl)-2-oxo-1,2-dihydropyridin-3-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-(4-fluoro-3,5-dimethylphenyl)-4-methyl-3-(2-oxo-1-(3-(trifluoromethyl)imidazo[1,5-a]pyridin-7-yl)-1,2-dihydropyridin-3-yl)-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-(1-(1-(difluoromethyl)-1H-indazol-5-yl)-2-oxo-1,2-dihydropyridin-3-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-(1-(1-(difluoromethyl)-1H-indazol-5-yl)-2-oxo-1,2-dihydropyridin-3-yl)-2-(4-fluoro-3,5-dimethylphenyl)-4-methyl-4,5,6,7-tetrahydro-2H-pyrazolo[4,3-c]pyridine-5-carbonyl)-5-((S)-2,2-dimethyltetrahydro-2H-pyran-4-yl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-2-(4-fluoro-3,5-dimethylphenyl)-4-methyl-3-(2′-methyl-2-oxo-2H-[1,4′-bipyridin]-3-yl)-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-2-(4-fluoro-3,5-dimethylphenyl)-3-(1-(imidazo[1,5-a]pyridin-6-yl)-2-oxo-1,2-dihydropyridin-3-yl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(2-((S)-3′-(2′-cyclopropoxy-2-oxo-2H-[1,4′-bipyridin]-3-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-(2′-cyclopropoxy-2-oxo-2H-[1,4′-bipyridin]-3-yl)-2-(4-fluoro-3,5-dimethylphenyl)-4,7,7-trimethyl-4,5,6,7-tetrahydro-2H-pyrazolo[4,3-c]pyridine-5-carbonyl)-5-((S)-2,2-dimethyltetrahydro-2H-pyran-4-yl)-1H-indol-1-yl)-2-methylcyclopropyl)-1,2,4-oxadiazol-5(4H)-one;
3-((1S,2S)-1-(2-((S)-3-(2′-cyclopropoxy-2-oxo-2H-[1,4′-bipyridin]-3-yl)-2-(4-fluoro-3,5-dimethylphenyl)-4,7,7-trimethyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3′-(2′-cyclopropoxy-2-oxo-2H-[1,4′-bipyridin]-3-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-5-((S)-2,2-dimethyltetrahydro-2H-pyran-4-yl)-1H-indol-1-yl)-2-methylcyclopropyl)-1,2,4-oxadiazol-5(4H)-one;
3-((1S,2S)-1-(2-((S)-3-(2′-cyclopropoxy-2-oxo-2H-[1,4′-bipyridin]-3-yl)-2-(4-fluoro-3,5-dimethylphenyl)-4-methyl-4,5,6,7-tetrahydro-2H-pyrazolo[4,3-c]pyridine-5-carbonyl)-5-((S)-2,2-dimethyltetrahydro-2H-pyran-4-yl)-1H-indol-1-yl)-2-methylcyclopropyl)-1,2,4-oxadiazol-5(4H)-one;
3-((1S,2S)-1-(2-((S)-3-(2′-cyclopropoxy-2-oxo-2H-[1,4′-bipyridin]-3-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one
3-((1S,2S)-1-(2-((S)-2-(4-fluoro-3,5-dimethylphenyl)-3-(1-(isoquinolin-6-yl)-2-oxo-1,2-dihydropyridin-3-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-(1-(4-(diethylphosphoryl)-3-(methylamino)phenyl)-2-oxo-1,2-dihydropyridin-3-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-(4-fluoro-3,5-dimethylphenyl)-4-methyl-3-(1-(4-morpholinophenyl)-2-oxo-1,2-dihydropyridin-3-yl)-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-7,7-difluoro-2-(4-fluoro-3,5-dimethylphenyl)-4-methyl-3-(1-(1-methyl-1H-indazol-5-yl)-2-oxo-1,2-dihydropyridin-3-yl)-4,5,6,7-tetrahydro-2H-pyrazolo[4,3-c]pyridine-5-carbonyl)-5-((S)-2,2-dimethyltetrahydro-2H-pyran-4-yl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-2-(4-fluoro-3,5-dimethylphenyl)-4,7,7-trimethyl-3-(1-(1-methyl-1H-indazol-5-yl)-6-oxo-1,6-dihydropyrimidin-5-yl)-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-2-(4-fluoro-3,5-dimethylphenyl)-4-methyl-3-(1-(1-methyl-1H-indazol-5-yl)-2-oxo-1,2-dihydropyridin-3-yl)-2,4,5,6,7,8-hexahydropyrazolo[4,3-c]azepine-5-carbonyl)-1H-indol-1-yl)-2-methylcyclopropyl)-1,2,4-oxadiazol-5(4H)-one;
3-((1S,2S)-1-(2-((S)-2-(4-fluoro-3,5-dimethylphenyl)-3-(1-(3-methoxyphenyl)-2-oxo-1,2-dihydropyridin-3-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-(4-fluoro-3,5-dimethylphenyl)-4-methyl-3-(2-oxo-1-(1-(2,2,2-trifluoroethyl)-1H-indazol-5-yl)-1,2-dihydropyridin-3-yl)-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-(1-(1-cyclopropyl-1H-indazol-5-yl)-2-oxo-1,2-dihydropyridin-3-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-(2-(4-fluoro-1-methyl-1H-indazol-5-yl)-3-oxo-2,3-dihydropyridazin-4-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-(4-fluoro-3,5-dimethylphenyl)-4-methyl-3-(2-(1-methyl-1H-indazol-5-yl)-3-oxo-2,3-dihydropyridazin-4-yl)-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-(4-fluoro-3,5-dimethylphenyl)-4,7,7-trimethyl-3-(2-(1-methyl-1H-indazol-5-yl)-3-oxo-2,3-dihydropyridazin-4-yl)-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2′-(4-fluoro-3,5-dimethylphenyl)-4′-methyl-3′-(2-(1-methyl-1H-indazol-5-yl)-3-oxo-2,3-dihydropyridazin-4-yl)-2′,4′,5′,6′-tetrahydrospiro[cyclopropane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-(4-fluoro-3,5-dimethylphenyl)-4-methyl-3-(1-(1-methyl-1H-indazol-5-yl)-6-oxo-1,6-dihydropyrimidin-5-yl)-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3′-(1-(4-fluoro-1-methyl-1H-indazol-5-yl)-2-oxo-1,2-dihydropyridin-3-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-3-(1-(4-fluoro-1-methyl-1H-indazol-5-yl)-6-oxo-1,6-dihydropyrimidin-5-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(2-((S)-3-(4-(4-fluoro-1-methyl-1H-indazol-5-yl)-5-oxo-4,5-dihydro-1H-1,2,4-triazol-1-yl)-2-(4-fluoro-3,5-dimethylphenyl)-4,7,7-trimethyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-(4-(4-fluoro-1-methyl-1H-indazol-5-yl)-5-oxo-4,5-dihydro-1H-1,2,4-triazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
(S)-3-(1-(2-(3-(4-(4-fluoro-1-methyl-1H-indazol-5-yl)-5-oxo-4,5-dihydro-1H-1,2,4-triazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)cyclopropyl)-1,2,4-oxadiazol-5(4H)-one;
3-((1S,2S)-1-(2-((S)-3′-(4-(4-fluoro-1-methyl-1H-indazol-5-yl)-5-oxo-4,5-dihydro-1H-1,2,4-triazol-1-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-3-(4-(4-fluoro-1-methyl-1H-indazol-5-yl)-5-oxo-4,5-dihydro-1H-1,2,4-triazol-1-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-3′-(4-(4-fluoro-1-methyl-1H-indazol-5-yl)-5-oxo-4,5-dihydro-1H-1,2,4-triazol-1-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-1H-indol-1-yl)-2-methylcyclopropyl)-1,2,4-oxadiazol-5(4H)-one;
N-(4-fluoro-1-methyl-1H-indazol-5-yl)-2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)acetamide;
2-((4'S)-5′-(5-(2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2′-(4-fluoro-3,5-dimethylphenyl)-4′-methyl-2′,4′,5′,6′-tetrahydrospiro[cyclobutane-1,7′-pyrazolo[4,3-c]pyridin]-3′-yl)-N-(4-fluoro-1-methyl-1H-indazol-5-yl)acetamide;
N-(4-fluoro-1-methyl-1H-indazol-5-yl)-2-((S)-2′-(4-fluoro-3,5-dimethylphenyl)-4′-methyl-5′-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-2′,4′,5′,6′-tetrahydrospiro[cyclobutane-1,7′-pyrazolo[4,3-c]pyridin]-3′-yl)acetamide;
2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N-(isochroman-7-yl)acetamide;
2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N-(4-(3-methyl-2-oxoimidazolidin-1-yl)phenyl)acetamide;
2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N-(1-methyl-1H-benzo[d]imidazol-6-yl)acetamide;
2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N-(1-methyl-1H-benzo[d][1,2,3]triazol-5-yl)acetamide;
2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N-(1-isopropyl-1H-indazol-5-yl)acetamide;
2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N-(4-morpholinophenyl)acetamide;
2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N-(2-methyl-2H-indazol-5-yl)acetamide;
2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N-(2-methylpyridin-4-yl)acetamide;
2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N-((1r,4S)-4-hydroxycyclohexyl)acetamide;
N-((1r,4S)-4-cyclopropoxycyclohexyl)-2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)acetamide;
N-(4,4-difluorocyclohexyl)-2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)acetamide
2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N-(4-(2-methoxyethoxy)cyclohexyl)acetamide;
2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N-(trans-4-methoxycyclohexyl)acetamide;
2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N-(1-methyl-1H-indazol-5-yl)acetamide;
2-((4S)-5-(5-(2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)-N-(4-fluoro-1-methyl-1H-indazol-5-yl)acetamide;
N-cyclohexyl-2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)acetamide;
N-(dibenzo[b,d,]furan-2-yl)-2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)acetamide
N-(4-(diethylphosphoryl)-3-(methylamino)phenyl)-2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)acetamide;
2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N—((R)-4,5,6,7-tetrahydropyrazolo[1,5-a]pyridin-5-yl)acetamide or 2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N—((S)-4,5,6,7-tetrahydropyrazolo[1,5-a]pyridin-5-yl)acetamide;
2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N—((R)-4,5,6,7-tetrahydropyrazolo[1,5-a]pyridin-5-yl)acetamide;
2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N—((S)-4,5,6,7-tetrahydropyrazolo[1,5-a]pyridin-5-yl)acetamide;
2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N-(4-(4-methyl-1H-imidazol-1-yl)phenyl)acetamide;
3-((1S,2S)-1-(2-((S)-3-(5-(4-fluoro-1-methyl-1H-indazol-5-yl)-1H-1,2,4-triazol-3-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-3-(5-(4-fluoro-1-methyl-1H-indazol-5-yl)-1H-1,2,4-triazol-3-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
(S)-3-(1-(2-(3-(5-(4-fluoro-1-methyl-1H-indazol-5-yl)-1H-1,2,4-triazol-3-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)cyclopropyl)-1,2,4-oxadiazol-5(4H)-one;
3-((1S,2S)-1-(2-((S)-3′-(3-(4-fluoro-1-methyl-1H-indazol-5-yl)-1H-pyrazol-1-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-3′-(3-(4-fluoro-1-methyl-1H-indazol-5-yl)-1H-pyrazol-1-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-1H-indol-1-yl)-2-methylcyclopropyl)-1,2,4-oxadiazol-5(4H)-one;
3-((1S,2S)-1-(2-((S)-3-(3-(4-fluoro-1-methyl-1H-indazol-5-yl)-1H-pyrazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
(S)-3-(1-(2-(3-(3-(4-fluoro-1-methyl-1H-indazol-5-yl)-1H-pyrazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)cyclopropyl)-1,2,4-oxadiazol-5(4H)-one;
2,2-difluoro-2-(4-fluoro-1-methyl-1H-indazol-5-yl)-N—((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)acetamide:
2,2-difluoro-2-(4-fluoro-1-methyl-1H-indazol-3-yl)-N—((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)acetamide;
N—((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)-2,2-difluoro-2-(4-fluoro-1-methyl-1H-indazol-5-yl)acetamide;
(S)-2-(4-fluoro-1-methyl-1H-indazol-5-yl)-N-(2-(4-fluoro-3,5-dimethylphenyl)-4-methyl-5-(1-(1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)acetamide;
2-(4-fluoro-1-methyl-1H-indazol-5-yl)-N—((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N-methylacetamide;
3-((1S,2S)-1-(2-((S)-3-((1S,5R)-4-(4-fluoro-1-methyl-1H-indazol-5-yl)-3-oxo-2,4-diazabicyclo[3.1.0]hexan-2-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-((1R,5S)-4-(4-fluoro-1-methyl-1H-indazol-5-yl)-3-oxo-2,4-diazabicyclo[3.1.0]hexan-2-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-((1S,5R)-4-(4-fluoro-1-methyl-1H-indazol-5-yl)-3-oxo-2,4-diazabicyclo[3.1.0]hexan-2-yl)-2-(4-fluoro-3,5-dimethylphenyl)-4,7,7-trimethyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-((1R,5S)-4-(4-fluoro-1-methyl-1H-indazol-5-yl)-3-oxo-2,4-diazabicyclo[3.1.0]hexan-2-yl)-2-(4-fluoro-3,5-dimethylphenyl)-4,7,7-trimethyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3′-((1S,5R)-4-(4-fluoro-1-methyl-1H-indazol-5-yl)-3-oxo-2,4-diazabicyclo[3.1.0]hexan-2-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3′-((1R,5S)-4-(4-fluoro-1-methyl-1H-indazol-5-yl)-3-oxo-2,4-diazabicyclo[3.1.0]hexan-2-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-(4-fluoro-3,5-dimethylphenyl)-4-methyl-3-((1S,5R)-4-(1-methyl-1H-indazol-5-yl)-3-oxo-2,4-diazabicyclo[3.1.0]hexan-2-yl)-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-(4-fluoro-3,5-dimethylphenyl)-4-methyl-3-((1R,5S)-4-(1-methyl-1H-indazol-5-yl)-3-oxo-2,4-diazabicyclo[3.1.0]hexan-2-yl)-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-2-(4-fluoro-3,5-dimethylphenyl)-4-methyl-3-((1S,5R)-4-(1-methyl-1H-indazol-5-yl)-3-oxo-2,4-diazabicyclo[3.1.0]hexan-2-yl)-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-2-(4-fluoro-3,5-dimethylphenyl)-4-methyl-3-((1R,5S)-4-(1-methyl-1H-indazol-5-yl)-3-oxo-2,4-diazabicyclo[3.1.0]hexan-2-yl)-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-3-((1S,5R)-4-(4-fluoro-1-methyl-1H-indazol-5-yl)-3-oxo-2,4-diazabicyclo[3.1.0]hexan-2-yl)-2-(4-fluoro-3,5-dimethylphenyl)-4,7,7-trimethyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-3-((1R,5S)-4-(4-fluoro-1-methyl-1H-indazol-5-yl)-3-oxo-2,4-diazabicyclo[3.1.0]hexan-2-yl)-2-(4-fluoro-3,5-dimethylphenyl)-4,7,7-trimethyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-3′-((1S,5R)-4-(4-fluoro-1-methyl-1H-indazol-5-yl)-3-oxo-2,4-diazabicyclo[3.1.0]hexan-2-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-3′-((1R,5S)-4-(4-fluoro-1-methyl-1H-indazol-5-yl)-3-oxo-2,4-diazabicyclo[3.1.0]hexan-2-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-1H-indol-1-yl)-2-methylcyclopropyl)-1,2,4-oxadiazol-5(4H)-one;
3-((1S,2S)-1-(2-((S)-3′-((1S,5R)-4-(4-(diethylphosphoryl)-3-(methylamino)phenyl)-3-oxo-2,4-diazabicyclo[3.1.0]hexan-2-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3′-((1R,5S)-4-(4-(diethylphosphoryl)-3-(methylamino)phenyl)-3-oxo-2,4-diazabicyclo[3.1.0]hexan-2-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3′-((1R,5S)-4-(4-fluoro-1-methyl-1H-indazol-5-yl)-3-oxo-2,4-diazabicyclo[3.1.0]hexan-2-yl)-2′-(4-fluoro-3,5-dimethylphenyl)-4′-methyl-2′,4′,5′,6′-tetrahydrospiro[cyclobutane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3′-((1S,5R)-4-(4-fluoro-1-methyl-1H-indazol-5-yl)-3-oxo-2,4-diazabicyclo[3.1.0]hexan-2-yl)-2′-(4-fluoro-3,5-dimethylphenyl)-4′-methyl-2′,4′,5′,6′-tetrahydrospiro[cyclobutane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-2-(4-fluoro-3,5-dimethylphenyl)-4-methyl-3-((1S,5R)-4-(1-methyl-1H-indazol-5-yl)-3-oxo-2,4-diazabicyclo[3.1.0]hexan-2-yl)-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-2-(4-fluoro-3,5-dimethylphenyl)-4-methyl-3-((1R,5S)-4-(1-methyl-1H-indazol-5-yl)-3-oxo-2,4-diazabicyclo[3.1.0]hexan-2-yl)-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(2-((S)-3-(1-(4-fluoro-1-methyl-1H-indazol-5-yl)-2-oxo-1,2,5,6-tetrahydropyridin-3-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-(3-(4-fluoro-1-methyl-1H-indazol-5-yl)-2-oxo-2,3-dihydro-1H-imidazol-1-yl)-2-(2-(6-fluoro-3,4-dihydroquinolin-1(2H)-yl)-2-oxoethyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-(3-(4-fluoro-1-methyl-1H-indazol-5-yl)-2-oxo-2,3-dihydro-1H-imidazol-1-yl)-4-methyl-2-(2-oxo-2-(4-azaspiro[2.6]nonan-4-yl)ethyl)-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-(2-(4-azadispiro[2.1.25.33]decan-4-yl)-2-oxoethyl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-(2-((1R,5S)-8-azabicyclo[3.2.1]octan-8-yl)-2-oxoethyl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-(2-((1R,4R)-7-azabicyclo[2.2.1]heptan-7-yl)-2-oxoethyl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-(2-((2S,5R)-2,5-dimethylpyrrolidin-1-yl)-2-oxoethyl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-(2-(3,4-dihydroquinolin-1(2H)-yl)-2-oxoethyl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
N-benzyl-N-(cyclopropylmethyl)-2-((S)-3-(3-(4-fluoro-1-methyl-1H-indazol-5-yl)-2-oxo-2,3-dihydro-1H-imidazol-1-yl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-2-yl)acetamide;
3-((1S,2S)-1-(2-((S)-2-(2-(4,4-difluoropiperidin-1-yl)-2-oxoethyl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-(3-(4-fluoro-1-methyl-1H-indazol-5-yl)-2-oxo-2,3-dihydro-1H-imidazol-1-yl)-4-methyl-2-(2-oxo-2-(4-azaspiro[2.5]octan-4-yl)ethyl)-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
2-((S)-3-(3-(4-fluoro-1-methyl-1H-indazol-5-yl)-2-oxo-2,3-dihydro-1H-imidazol-1-yl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-2-yl)-N-(3-(trifluoromethyl)oxetan-3-yl)acetamide;
N-(bicyclo[2.2.2]octan-1-yl)-2-((S)-3-(3-(4-fluoro-1-methyl-1H-indazol-5-yl)-2-oxo-2,3-dihydro-1H-imidazol-1-yl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-2-yl)acetamide;
3-((1S,2S)-1-(2-((S)-3′-(3-(4-fluoro-1-methyl-1H-indazol-5-yl)-2-oxoimidazolidin-1-yl)-2′-(5-fluoro-4,6-dimethylpyrimidin-2-yl)-4′-methyl-2′,4′,5′,6′-tetrahydrospiro[cyclopropane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3′-(3-(4-fluoro-1-methyl-1H-indazol-5-yl)-2-oxoimidazolidin-1-yl)-1′-(5-fluoro-4,6-dimethylpyrimidin-2-yl)-4′-methyl-1′,4′,5′,6′-tetrahydrospiro[cyclopropane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
(S)-3-(1-(5-(3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-3-(isochroman-6-yl)-1H-pyrazol-1-yl)cyclopropyl)-1,2,4-oxadiazol-5(4H)-one;
3-(1-(5-((S)-2,2-dimethyltetrahydro-2H-pyran-4-yl)-2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)phenyl)cyclopropyl)-1,2,4-oxadiazol-5(4H)-one;
3-(1-(5-((R)-2,2-dimethyltetrahydro-2H-pyran-4-yl)-2-((S)-3-(3-(4-fluoro-1-methyl-H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)phenyl)cyclopropyl)-1,2,4-oxadiazol-5(4H)-one;
(S)-3-(1-(5-(3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-3-((tetrahydro-2H-pyran-4-yl)ethynyl)-1H-pyrazol-1-yl)cyclopropyl)-1,2,4-oxadiazol-5(4H)-one;
(S)-3-(1-(5-(3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-3-(4-(1-methoxycyclobutyl)phenyl)-1H-pyrazol-1-yl)cyclopropyl)-1,2,4-oxadiazol-5(4H)-one;
3-(1-(2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-((3aR,6aR)-hexahydro-1H-cyclopenta[c]furan-5-yl)phenyl)cyclopropyl)-1,2,4-oxadiazol-5(4H)-one;
3-(1-(2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-((3aS,6aS)-hexahydro-1H-cyclopenta[c]furan-5-yl)phenyl)cyclopropyl)-1,2,4-oxadiazol-5(4H)-one;
(S)-3-(1-(2-(3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(2-oxaspiro[3.5]nonan-7-yl)phenyl)cyclopropyl)-1,2,4-oxadiazol-5(4H)-one;
3-(1-(2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-((3aR,6aR)-hexahydro-1H-cyclopenta[c]furan-5-yl)-1H-indol-1-yl)cyclopropyl)-1,2,4-oxadiazol-5(4H)-one;
3-(1-(2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-((3aS,6aS)-hexahydro-1H-cyclopenta[c]furan-5-yl)-1H-indol-1-yl)cyclopropyl)-1,2,4-oxadiazol-5(4H)-one;
3-(1-(2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-((R)-2,5-dioxaspiro[3.5]nonan-7-yl)-1H-indol-1-yl)cyclopropyl)-1,2,4-oxadiazol-5(4H)-one;
3-(1-(2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-((S)-2,5-dioxaspiro[3.5]nonan-7-yl)-1H-indol-1-yl)cyclopropyl)-1,2,4-oxadiazol-5(4H)-one;
3-(1-(2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(2-hydroxybicyclo[3.2.1]octan-6-yl)-1H-indol-1-yl)cyclopropyl)-1,2,4-oxadiazol-5(4H)-one;
(S)-3-(1-(2-(3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(4-methoxycyclohexyl)-1H-indol-1-yl)cyclopropyl)-1,2,4-oxadiazol-5(4H)-one;
N-(4-fluoro-1-methyl-1H-indazol-5-yl)-2-((S)-2′-(4-fluoro-3,5-dimethylphenyl)-4′-methyl-5′-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-2′,4′,5′,6′-tetrahydrospiro[cyclopropane-1,7′-pyrazolo[4,3-c]pyridin]-3′-yl)acetamide;
2-((S)-5′-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2′-(4-fluoro-3,5-dimethylphenyl)-4′-methyl-2′,4′,5′,6′-tetrahydrospiro[cyclopropane-1,7′-pyrazolo[4,3-c]pyridin]-3′-yl)-N-(4-fluoro-1-methyl-1H-indazol-5-yl)acetamide;
2-((S)-5′-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2′-(4-fluoro-3,5-dimethylphenyl)-4′-methyl-2′,4′,5′,6′-tetrahydrospiro[cyclopropane-1,7′-pyrazolo[4,3-c]pyridin]-3′-yl)-N-(1-methyl-1H-indazol-5-yl)acetamide;
2-((S)-2′-(4-fluoro-3,5-dimethylphenyl)-4′-methyl-5′-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-2′,4′,5′,6′-tetrahydrospiro[cyclopropane-1,7′-pyrazolo[4,3-c]pyridin]-3′-yl)-N-(1-methyl-1H-indazol-5-yl)acetamide;
2-((S)-2-(4-fluoro-3,5-dimethylphenyl)-4,7,7-trimethyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N-(1-methyl-1H-indazol-5-yl)acetamide;
N-(4-fluoro-1-methyl-1H-indazol-5-yl)-2-((S)-2-(4-fluoro-3,5-dimethylphenyl)-4,7,7-trimethyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)acetamide;
2,2-difluoro-N-(4-fluoro-1-methyl-1H-indazol-5-yl)-2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)acetamide;
3-((1S,2S)-1-(2-((S)-3-(1-(4-fluoro-1-methyl-1H-indazol-5-yl)-5-oxo-1,5-dihydro-4H-1,2,4-triazol-4-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-((S)-1-(4-fluoro-1-methyl-1H-indazol-5-yl)-2-oxopiperidin-3-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-((R)-1-(4-fluoro-1-methyl-1H-indazol-5-yl)-2-oxopiperidin-3-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-((1S,6S)-3-(4-fluoro-1-methyl-1H-indazol-5-yl)-2-oxo-3-azabicyclo[4.1.0]heptan-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-((1R,6R)-3-(4-fluoro-1-methyl-1H-indazol-5-yl)-2-oxo-3-azabicyclo[4.1.0]heptan-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-3-((1R,6R)-3-(4-fluoro-1-methyl-1H-indazol-5-yl)-2-oxo-3-azabicyclo[4.1.0]heptan-1-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-3-((1S,6S)-3-(4-fluoro-1-methyl-1H-indazol-5-yl)-2-oxo-3-azabicyclo[4.1.0]heptan-1-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one
3-((1S,2S)-1-(5-((S)-2,2-dimethyltetrahydro-2H-pyran-4-yl)-2-((S)-3-((1R,6R)-3-(4-fluoro-1-methyl-1H-indazol-5-yl)-2-oxo-3-azabicyclo[4.1.0]heptan-1-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-3-((1S,6S)-3-(4-fluoro-1-methyl-1H-indazol-5-yl)-2-oxo-3-azabicyclo[4.1.0]heptan-1-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(2-((S)-3-((1R,6R)-3-(4-fluoro-1-methyl-1H-indazol-5-yl)-2-oxo-3-azabicyclo[4.1.0]heptan-1-yl)-2-(4-fluoro-3,5-dimethylphenyl)-4,7,7-trimethyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-((1S,6S)-3-(4-fluoro-1-methyl-1H-indazol-5-yl)-2-oxo-3-azabicyclo[4.1.0]heptan-1-yl)-2-(4-fluoro-3,5-dimethylphenyl)-4,7,7-trimethyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-((1R,6R)-3-(4-fluoro-1-methyl-1H-indazol-5-yl)-2-oxo-3-azabicyclo[4.1.0]heptan-1-yl)-2-(4-fluoro-3,5-dimethylphenyl)-4,7,7-trimethyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-((1S,6S)-3-(4-fluoro-1-methyl-1H-indazol-5-yl)-2-oxo-3-azabicyclo[4.1.0]heptan-1-yl)-2-(4-fluoro-3,5-dimethylphenyl)-4,7,7-trimethyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-(5-(4-fluoro-1-methyl-1H-indazol-5-yl)-1H-pyrazol-3-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((Z)—((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-2,4,6,7-tetrahydro-5H-pyrazolo[4,3-c]pyridin-5-yl)(methylimino)methyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((Z)-(ethylimino)((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-2,4,6,7-tetrahydro-5H-pyrazolo[4,3-c]pyridin-5-yl)methyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((Z)-((cyclopropylmethyl)imino)((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-2,4,6,7-tetrahydro-5H-pyrazolo[4,3-c]pyridin-5-yl)methyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-(4-(4-fluoro-1-methyl-1H-indazol-5-yl)-1H-pyrazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
(2S,4S)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-6,11-dioxo-N-(pyrrolidin-3-yl)-1,2,3,4,6,11-hexahydrotetracene-2-carboxamide;
3-((1S,2S)-1-(2-((S)-3-(1-(4-fluoro-1-methyl-1H-indazol-5-yl)-1H-pyrazol-4-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one; and
3-((1S,2S)-1-(5-((S)-2,2-dimethyltetrahydro-2H-pyran-4-yl)-2-((S)-3-(3-(4-fluoro-1-methyl-1H-indazol-5-yl)-2-oxopyridin-1(2H)-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one,
   or a pharmaceutically acceptable salt thereof.

In embodiment no. 134, the present invention provides a compound selected from:
3-[(1S,2S)-1-[5-[(4S)-2,2-dimethyltetrahydropyran-4-yl]-2-[(4S)-2-(4-fluoro-3,5-dimethyl-phenyl)-4-methyl-3-[3-(3-methylbenzimidazol-1-ium-5-yl)-2-oxo-imidazol-1-yl]-4,6,7,8-tetrahydropyrazolo[4,3-c]azepine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-4-methyl-3-[3-(1-methylbenzotriazol-5-yl)-2-oxo-imidazol-1-yl]-4,6,7,8-tetrahydropyrazolo[4,3-c]azepin-1-ium-5-carbonyl]indol-1-yl]-2-methyl-cyclopropyl]-4H-1,2,4-oxadiazol-5-one;
3-[(1S,2S)-1-[2-[(4S)-3-[3-(2,3-dimethylimidazo[1,2-a]pyridin-1-ium-6-yl)-2-oxo-imidazol-1-yl]-2-(4-fluoro-3,5-dimethyl-phenyl)-4-methyl-4,6,7,8-tetrahydropyrazolo[4,3-c]azepine-5-carbonyl]-5-[(4S)-2,2-dimethyltetrahydropyran-4-yl]indol-1-yl]-2-methyl-cyclopropyl]-4H-1,2,4-oxadiazol-5-one;
3-[(1S,2S)-1-[2-[(4S)-3-[3-[2-(difluoromethyl)indazol-5-yl]-2-oxo-imidazol-1-yl]-2-(4-fluoro-3,5-dimethyl-phenyl)-4-methyl-4,6,7,8-tetrahydropyrazolo[4,3-c]azepin-1-ium-5-carbonyl]-5-[(4S)-2,2-dimethyltetrahydropyran-4-yl]indol-1-yl]-2-methyl-cyclopropyl]-4H-1,2,4-oxadiazol-5-one;
3-[(1S,2S)-1-[2-[(4S)-3-[3-(2,3-dimethylpyridin-1-ium-4-yl)-2-oxo-imidazol-1-yl]-2-(4-fluoro-3,5-dimethyl-phenyl)-4-methyl-4,6,7,8-tetrahydropyrazolo[4,3-c]azepine-5-carbonyl]-5-[(4S)-2,2-dimethyltetrahydropyran-4-yl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-4-methyl-3-[3-(1-methylisoquinolin-2-ium-6-yl)-2-oxo-imidazol-1-yl]-4,6,7,8-tetrahydropyrazolo[4,3-c]azepine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[3-(4-fluoro-1-methyl-indazol-5-yl)-2-oxo-imidazol-1-yl]-4-methyl-spiro[6,8-dihydro-4H-pyrazolo[4,3-c]azepin-1-ium-7,1′-cyclopropane]-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(5-fluoro-4,6-dimethyl-pyridin-1-ium-2-yl)-3-[3-(4-fluoro-1-methyl-indazol-5-yl)-2-oxo-imidazol-1-yl]-4-methyl-4,6,7,8-tetrahydropyrazolo[4,3-c]azepine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-4-methyl-3-[2-oxo-3-[2-(2,2,2-trifluoroethyl)indazol-5-yl]imidazol-1-yl]-4,6,7,8-tetrahydropyrazolo[4,3-c]azepin-1-ium-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-4-methyl-3-[3-(1-methylindazol-5-yl)-2-oxo-imidazol-1-yl]-4,6,7,8-tetrahydropyrazolo[4,3-c]azepin-1-ium-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-4-methyl-3-[2-oxo-3-[1-(trideuteriomethyl)indazol-5-yl]imidazol-1-yl]-4,6,7,8-tetrahydropyrazolo[4,3-c]azepin-1-ium-5-carbonyl]indol-1-yl]-2-methyl-cyclopropyl]-4H-1,2,4-oxadiazol-5-one;
3-[(1S,2S)-1-[2-[(4S)-3-[3-(1-cyclopropylindazol-5-yl)-2-oxo-imidazol-1-yl]-2-(4-fluoro-3,5-dimethyl-phenyl)-4-methyl-4,6,7,8-tetrahydropyrazolo[4,3-c]azepin-1-ium-5-carbonyl]-5-[(4S)-2,2-dimethyltetrahydropyran-4-yl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[3-(4-fluoro-1-methyl-indazol-5-yl)-2-oxo-imidazol-1-yl]-4,7,7-trimethyl-6,8-dihydro-4H-pyrazolo[4,3-c]azepine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-4-methyl-3-[2-oxo-3-[1-(2,2,2-trifluoroethyl)indazol-5-yl]imidazol-1-yl]-4,6,7,8-tetrahydropyrazolo[4,3-c]azepin-1-ium-5-carbonyl]indol-1-yl]-2-methyl-cyclopropyl]-4H-1,2,4-oxadiazol-5-one;
3-[(1S,2S)-1-[5-(2,2-dimethylmorpholin-4-ium-4-yl)-2-[(4S)-4-ethyl-2-(4-fluoro-3,5-dimethyl-phenyl)-3-[3-(4-fluoro-1-methyl-indazol-5-yl)-2-oxo-imidazol-1-yl]-4,6,7,8-tetrahydropyrazolo[4,3-c]azepine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-3-[3-(4-fluoro-1-methyl-indazol-5-yl)-2-oxo-imidazol-1-yl]-4-methyl-2-[5-(trifluoromethyl)-3-pyridyl]-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[3-(4-fluoro-1-methyl-indazol-5-yl)-2-oxo-imidazol-1-yl]-4-methyl-spiro[4,6-dihydropyrazolo[4,3-c]pyridine-7,4′-tetrahydropyran]-5-carbonyl]indol-1-yl]-2-methyl-cyclopropyl]-4H-1,2,4-oxadiazol-5-one;
calcium 3-[(1S,2S)-1-[5-[(4S)-2,2-dimethyltetrahydropyran-4-yl]-2-[(4S)-2-(4-fluoro-3,5-dimethyl-phenyl)-4-methyl-3-[1-(2-methyl-6-quinolyl)-2-oxo-3-pyridyl]-6,7-dihydro-4H-pyrazolo[4,3-c]pyridine-5-carbonyl]indol-1-yl]-2-methyl-cyclopropyl]-1-oxa-2-aza-4-azanidacyclopent-2-en-5-one;
3-[(1S,2S)-1-[2-[(4S)-2-(4-fluoro-3,5-dimethyl-phenyl)-4-methyl-3-[1-(2-methyl-6-quinolyl)-2-oxo-3-pyridyl]-6,7-dihydro-4H-pyrazolo[4,3-c]pyridine-5-carbonyl]-5-[(2S)-2-methylmorpholin-4-yl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-4-methyl-3-[1-(2-methyl-6-quinolyl)-6-oxo-pyrimidin-5-yl]-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[1-(4-fluoro-1-methyl-indazol-5-yl)-6-oxo-pyrimidin-5-yl]-4-methyl-4,6,7,8-tetrahydropyrazolo[4,3-c]azepin-1-ium-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-4-methyl-3-[1-(1-methylisoquinolin-2-ium-6-yl)-2-oxo-3-pyridyl]-4,6,7,8-tetrahydropyrazolo[4,3-c]azepine-5-carbonyl]indol-1-yl]-2-methyl-cyclopropyl]-4H-1,2,4-oxadiazol-5-one;
3-[(1S,2S)-1-[2-[(4S)-3-[1-[2-(cyclopropoxy)pyridin-1-ium-4-yl]-2-oxo-3-pyridyl]-2-(4-fluoro-3,5-dimethyl-phenyl)-4-methyl-4,6,7,8-tetrahydropyrazolo[4,3-c]azepine-5-carbonyl]-5-[(4S)-2,2-dimethyltetrahydropyran-4-yl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[1-(4-fluoro-1-methyl-indazol-5-yl)-2-oxo-3-pyridyl]-4-methyl-4,6,7,8-tetrahydropyrazolo[4,3-c]azepin-1-ium-5-carbonyl]indol-1-yl]-2-methyl-cyclopropyl]-4H-1,2,4-oxadiazol-5-one;
3-[(1S,2S)-1-[2-[(4S)-2-(4-fluoro-3,5-dimethyl-phenyl)-3-[1-(4-fluoro-1-methyl-indazol-2-ium-5-yl)-2-oxo-3-pyridyl]-4-methyl-spiro[4,6-dihydropyrazolo[4,3-c]pyridine-7,1′-cyclobutane]-5-carbonyl]-5-tetrahydropyran-4-yl-indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[1-(5-fluoro-2-methyl-6-quinolyl)-2-oxo-3-pyridyl]-4-methyl-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[1-(4-fluoro-1-methyl-indazol-5-yl)-2-oxo-3-pyridyl]-4-methyl-1′-(2,2,2-trifluoroethyl)spiro[4,6-dihydropyrazolo[4,3-c]pyridine-7,3′-azetidin-1-ium]-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[1-(5-fluoro-1-methyl-isoquinolin-2-ium-6-yl)-2-oxo-3-pyridyl]-4-methyl-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[1-(4-fluoro-1-methyl-indazol-2-ium-5-yl)-2-oxo-3-pyridyl]-4-methyl-spiro[4,6-dihydropyrazolo[4,3-c]pyridine-7,1′-cyclobutane]-5-carbonyl]indol-1-yl]-2-methyl-cyclopropyl]-4H-1,2,4-oxadiazol-5-one;
N-(1-cyclopropyl-4-fluoro-indazol-5-yl)-2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-spiro[6,8-dihydro-4H-pyrazolo[4,3-c]azepin-1-ium-7,1′-cyclopropane]-3-yl]acetamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-spiro[6,8-dihydro-4H-pyrazolo[4,3-c]azepin-1-ium-7,1′-cyclopropane]-3-yl]-N-(1-methylindazol-5-yl)acetamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-spiro[6,8-dihydro-4H-pyrazolo[4,3-c]azepin-1-ium-7,1′-cyclopropane]-3-yl]-N-(6-fluoro-1-methyl-indazol-5-yl)acetamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-spiro[6,8-dihydro-4H-pyrazolo[4,3-c]azepin-1-ium-7,1′-cyclopropane]-3-yl]-N-(4-fluoro-1-methyl-indazol-5-yl)acetamide;
N-(1-methylindazol-5-yl)-2-[(4S)-4,7,7-trimethyl-5-[1-[(1S,2S)-2-methyl-1-(5-oxo-4H-1,2,4-oxadiazol-3-yl)cyclopropyl]-5-tetrahydropyran-4-yl-indole-2-carbonyl]-2-[1-methyl-5-(trifluoromethyl)pyrazol-3-yl]-4,6-dihydropyrazolo[4,3-c]pyridin-1-ium-3-yl]acetamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-4,7,7-trimethyl-2-[1-methyl-5-(trifluoromethyl)pyrazol-3-yl]-4,6-dihydropyrazolo[4,3-c]pyridin-1-ium-3-yl]-N-(1-methylindazol-5-yl)acetamide;
2-[(4S)-2-(4-fluoro-3,5-dimethyl-phenyl)-4-methyl-5-[1-[(1S,2S)-2-methyl-1-(5-oxo-4H-1,2,4-oxadiazol-3-yl)cyclopropyl]-5-tetrahydropyran-4-yl-indole-2-carbonyl]-6,7-dihydro-4H-pyrazolo[4,3-c]pyridin-3-yl]-N-(2-methylquinolin-1-ium-6-yl)acetamide;
2-[(4S)-2-(4-fluoro-3,5-dimethyl-phenyl)-4,7,7-trimethyl-5-[5-[(2S)-2-methylmorpholin-4-yl]-1-[(1S,2S)-2-methyl-1-(5-oxo-4H-1,2,4-oxadiazol-3-yl)cyclopropyl]indole-2-carbonyl]-4,6-dihydropyrazolo[4,3-c]pyridin-3-yl]-N-(2-methylquinolin-1-ium-6-yl)acetamide;
2-[(4S)-2-(4-fluoro-3,5-dimethyl-phenyl)-4,7,7-trimethyl-5-[5-[(2S)-2-methylmorpholin-4-ium-4-yl]-1-[(1S,2S)-2-methyl-1-(5-oxo-4H-1,2,4-oxadiazol-3-yl)cyclopropyl]indole-2-carbonyl]-4,6-dihydropyrazolo[4,3-c]pyridin-3-yl]-N-(1-methylindazol-5-yl)acetamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4,7,7-trimethyl-4,6-dihydropyrazolo[4,3-c]pyridin-1-ium-3-yl]-N-(4-fluoro-1-methyl-indazol-5-yl)acetamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4,7,7-trimethyl-4,6-dihydropyrazolo[4,3-c]pyridin-3-yl]-N-(2-methylquinolin-1-ium-6-yl)acetamide;
2-[(4S)-2-(4-fluoro-3,5-dimethyl-phenyl)-4-methyl-5-[1-[(1S,2S)-2-methyl-1-(5-oxo-4H-1,2,4-oxadiazol-3-yl)cyclopropyl]-5-tetrahydropyran-4-yl-indole-2-carbonyl]-6,7-dihydro-4H-pyrazolo[4,3-c]pyridin-1-ium-3-yl]-N-(4-methoxy-1-bicyclo[2.2.2]octanyl)acetamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4,7,7-trimethyl-4,6-dihydropyrazolo[4,3-c]pyridin-1-ium-3-yl]-N-(1-methylindazol-5-yl)acetamide;
2-[(4S)-2-(4-fluoro-3,5-dimethyl-phenyl)-4-methyl-5-[5-[(2S)-2-methylmorpholin-4-ium-4-yl]-1-[(1S,2S)-2-methyl-1-(5-oxo-4H-1,2,4-oxadiazol-3-yl)cyclopropyl]indole-2-carbonyl]-6,7-dihydro-4H-pyrazolo[4,3-c]pyridin-3-yl]-N-(4-fluoro-1-methyl-indazol-5-yl)acetamide;
2-[(4S)-2-(4-fluoro-3,5-dimethyl-phenyl)-4-methyl-5-[5-[(2R)-2-methylmorpholin-4-ium-4-yl]-1-[(1S,2S)-2-methyl-1-(5-oxo-4H-1,2,4-oxadiazol-3-yl)cyclopropyl]indole-2-carbonyl]-6,7-dihydro-4H-pyrazolo[4,3-c]pyridin-3-yl]-N-(4-fluoro-1-methyl-indazol-5-yl)acetamide;
N-[4-(cyclopropoxy)cyclohexyl]-2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4,7,7-trimethyl-4,6-dihydropyrazolo[4,3-c]pyridin-1-ium-3-yl]acetamide;
3-[(1S,2S)-1-[5-[(4S)-2,2-dimethyltetrahydropyran-4-yl]-2-[(4S)-2-(4-fluoro-3,5-dimethyl-phenyl)-3-[4-(4-fluoro-1-methyl-indazol-5-yl)-3-oxo-2,4-diazabicyclo[3.1.0]hexan-2-yl]-4-methyl-4,6,7,8-tetrahydropyrazolo[4,3-c]azepin-1-ium-5-carbonyl]indol-1-yl]-2-methyl-cyclopropyl]-4H-1,2,4-oxadiazol-5-one;
3-[1-[2-[(4S)-2-(4-fluoro-3,5-dimethyl-phenyl)-3-[3-(4-fluoro-1-methyl-indazol-5-yl)-2-oxo-imidazol-1-yl]-4-methyl-6,7-dihydro-4H-pyrazolo[4,3-c]pyridine-5-carbonyl]-5-[(1S,5R)-6-hydroxy-2-bicyclo[3.2.1]octanyl]indol-1-yl]cyclopropyl]-4H-1,2,4-oxadiazol-5-one;
3-[1-[2-[(4S)-2-(4-fluoro-3,5-dimethyl-phenyl)-3-[3-(4-fluoro-1-methyl-indazol-5-yl)-2-oxo-imidazol-1-yl]-4-methyl-6,7-dihydro-4H-pyrazolo[4,3-c]pyridine-5-carbonyl]-5-[(1S,5R)-6-hydroxy-2-bicyclo[3.2.1]octanyl]indol-1-yl]cyclopropyl]-4H-1,2,4-oxadiazol-5-one;
3-[(1S,2S)-1-[2-[(4S)-2-(4-fluoro-3,5-dimethyl-phenyl)-3-[3-(4-fluoro-1-methyl-indazol-5-yl)-2-oxo-imidazol-1-yl]-4-methyl-4,6,7,8-tetrahydropyrazolo[4,3-c]azepine-5-carbonyl]-5-(4-oxa-7-azoniaspiro[2.5]octan-7-yl)indol-1-yl]-2-methyl-cyclopropyl]-4H-1,2,4-oxadiazol-5-one;
3-[(1S,2S)-1-[2-[(4S)-2-(4-fluoro-3,5-dimethyl-phenyl)-3-[3-(4-fluoro-1-methyl-indazol-5-yl)-2-oxo-imidazol-1-yl]-4-methyl-4,6,7,8-tetrahydropyrazolo[4,3-c]azepine-5-carbonyl]-5-[(2S)-2-methylmorpholin-4-ium-4-yl]indol-1-yl]-2-methyl-cyclopropyl]-4H-1,2,4-oxadiazol-5-one;
3-[(1S,2S)-1-[5-(2,2-dimethylmorpholin-4-ium-4-yl)-2-[(4S)-2-(4-fluoro-3,5-dimethyl-phenyl)-3-[3-(4-fluoro-1-methyl-indazol-5-yl)-2-oxo-imidazol-1-yl]-4-methyl-4,6,7,8-tetrahydropyrazolo[4,3-c]azepine-5-carbonyl]indol-1-yl]-2-methyl-cyclopropyl]-4H-1,2,4-oxadiazol-5-one;
3-[1-[3-(4,4-difluoroisochroman-6-yl)-5-[(4S)-2-(4-fluoro-3,5-dimethyl-phenyl)-3-[3-(4-fluoro-1-methyl-indazol-5-yl)-2-oxo-imidazol-1-yl]-4-methyl-6,7-dihydro-4H-pyrazolo[4,3-c]pyridine-5-carbonyl]pyrazol-1-yl]cyclopropyl]-4H-1,2,4-oxadiazol-5-one;
3-[(1S,2S)-1-[3-(1,7-dimethylindazol-2-ium-6-yl)-5-[(4S)-2-(4-fluoro-3,5-dimethyl-phenyl)-3-[3-(4-fluoro-1-methyl-indazol-5-yl)-2-oxo-imidazol-1-yl]-4-methyl-6,7-dihydro-4H-pyrazolo[4,3-c]pyridine-5-carbonyl]pyrazol-1-yl]-2-methyl-cyclopropyl]-4H-1,2,4-oxadiazol-5-one;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-2,2-difluoro-N-(4-fluoro-1-methyl-indazol-5-yl)acetamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-1-ium-3-yl]-N-(4-fluoro-1-methyl-indazol-5-yl)acetamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-1′-(2,2,2-trifluoroethyl)spiro[4,6-dihydropyrazolo[4,3-c]pyridine-7,3′-azetidin-1-ium]-3-yl]-N-(4-fluoro-1-methyl-indazol-5-yl)acetamide;
3-[(1S,2S)-1-[5-[(4S)-2,2-dimethyltetrahydropyran-4-yl]-2-[(4S)-2-(4-fluoro-3,5-dimethyl-phenyl)-3-[(1S,6S)-3-(4-fluoro-1-methyl-indazol-5-yl)-2-oxo-3-azabicyclo[4.1.0]heptan-1-yl]-4-methyl-spiro[4,6-dihydropyrazolo[4,3-c]pyridine-7,1′-cyclopropane]-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[(1R,6R)-3-(4-fluoro-1-methyl-indazol-5-yl)-2-oxo-3-azabicyclo[4.1.0]heptan-1-yl]-4-methyl-spiro[4,6-dihydropyrazolo[4,3-c]pyridine-7,1′-cyclopropane]-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[3-(4-fluoro-1-methyl-indazol-5-yl)-2-oxo-3-azabicyclo[4.1.0]heptan-1-yl]-4-methyl-4,6,7,8-tetrahydropyrazolo[4,3-c]azepine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[3-(4-fluoro-1-methyl-indazol-5-yl)-2-oxo-3-azabicyclo[4.1.0]heptan-1-yl]-4-methyl-4,6,7,8-tetrahydropyrazolo[4,3-c]azepine-5-carbonyl]indol-1-yl]-2-methyl-cyclopropyl]-4H-1,2,4-oxadiazol-5-one;
1-[(4S)-2-(4-fluoro-3,5-dimethyl-phenyl)-4-methyl-5-[1-[(1S,2S)-2-methyl-1-(5-oxo-4H-1,2,4-oxadiazol-3-yl)cyclopropyl]-5-tetrahydropyran-4-yl-indole-2-carbonyl]-6,7-dihydro-4H-pyrazolo[4,3-c]pyridin-3-yl]-N-(1-methylindazol-5-yl)cyclopropanecarboxamide;
3-[(1S,2S)-1-[2-[(4S)-2-(4-fluoro-3,5-dimethyl-phenyl)-4-methyl-3-[1-(1-methylindazol-5-yl)-2-oxo-pyrrolidin-3-yl]-6,7-dihydro-4H-pyrazolo[4,3-c]pyridine-5-carbonyl]-5-tetrahydropyran-4-yl-indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-4-methyl-3-[(1S,6S)-3-(2-methyl-6-quinolyl)-2-oxo-3-azabicyclo[4.1.0]heptan-1-yl]-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-4-methyl-3-[(1S,6S)-3-(1-methyl-6-isoquinolyl)-2-oxo-3-azabicyclo[4.1.0]heptan-1-yl]-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[2-[(4S)-3-[(1S,6S)-3-[2-(cyclopropoxy)-4-pyridyl]-2-oxo-3-azabicyclo[4.1.0]heptan-1-yl]-2-(4-fluoro-3,5-dimethyl-phenyl)-4-methyl-6,7-dihydro-4H-pyrazolo[4,3-c]pyridine-5-carbonyl]-5-[(4S)-2,2-dimethyltetrahydropyran-4-yl]indol-1-yl]-2-methyl-cyclopropyl]-4H-1,2,4-oxadiazol-5-one;
3-[(1S,2S)-1-[2-[(4S)-3-[1-[4-(cyclopropoxy)cyclohexyl]-6-oxo-pyrimidin-5-yl]-2-(4-fluoro-3,5-dimethyl-phenyl)-4-methyl-6,7-dihydro-4H-pyrazolo[4,3-c]pyridine-5-carbonyl]-5-[(4S)-2,2-dimethyltetrahydropyran-4-yl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[1-(4-methoxycyclohexyl)-6-oxo-pyrimidin-5-yl]-4-methyl-4,6,7,8-tetrahydropyrazolo[4,3-c]azepin-1-ium-5-carbonyl]indol-1-yl]-2-methyl-cyclopropyl]-4H-1,2,4-oxadiazol-5-one;
3-[(1S,2S)-1-[2-[(4S)-1′-(2,2-dimethylpropanoyl)-2-(4-fluoro-3,5-dimethyl-phenyl)-3-[3-(4-fluoro-1-methyl-indazol-5-yl)-2-oxo-imidazol-1-yl]-4-methyl-spiro[4,6-dihydropyrazolo[4,3-c]pyridine-7,3′-azetidine]-5-carbonyl]-5-tetrahydropyran-4-yl-indol-1-yl]-2-methyl-cyclopropyl]-4H-1,2,4-oxadiazol-5-one;
3-[(1S,2S)-1-[2-[(4S)-1′-(2,2-difluoroacetyl)-2-(4-fluoro-3,5-dimethyl-phenyl)-3-[3-(4-fluoro-1-methyl-indazol-5-yl)-2-oxo-imidazol-1-yl]-4-methyl-spiro[4,6-dihydropyrazolo[4,3-c]pyridine-7,3′-azetidine]-5-carbonyl]-5-tetrahydropyran-4-yl-indol-1-yl]-2-methyl-cyclopropyl]-4H-1,2,4-oxadiazol-5-one;
3-[(1S,2S)-1-[2-[(4S)-1′-(2,2-difluoropropanoyl)-2-(4-fluoro-3,5-dimethyl-phenyl)-3-[3-(4-fluoro-1-methyl-indazol-5-yl)-2-oxo-imidazol-1-yl]-4-methyl-spiro[4,6-dihydropyrazolo[4,3-c]pyridine-7,3′-azetidine]-5-carbonyl]-5-[(4S)-2,2-dimethyltetrahydropyran-4-yl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[3-(4-fluoro-1-methyl-indazol-5-yl)-2-oxo-imidazol-1-yl]-4-methyl-1′-(2,2,3,3-tetrafluoropropanoyl)spiro[4,6-dihydropyrazolo[4,3-c]pyridine-7,3′-azetidine]-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[3-(4-fluoro-1-methyl-indazol-5-yl)-2-oxo-imidazol-1-yl]-4-methyl-1′-(2,2,2-trifluoroacetyl)spiro[4,6-dihydropyrazolo[4,3-c]pyridine-7,3′-azetidine]-5-carbonyl]indol-1-yl]-2-methyl-cyclopropyl]-4H-1,2,4-oxadiazol-5-one;
3-[(1S,2S)-1-[2-[(4S)-1-acetyl-2-(4-fluoro-3,5-dimethyl-phenyl)-3-[3-(4-fluoro-1-methyl-indazol-5-yl)-2-oxo-imidazol-1-yl]-4-methyl-spiro[4,6-dihydropyrazolo[4,3-c]pyridine-7,3′-azetidine]-5-carbonyl]-5-tetrahydropyran-4-yl-indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[3-(4-fluoro-1-methyl-indazol-5-yl)-2-oxo-imidazol-1-yl]-4-methyl-1′-(2,2,2-trifluoroethyl)spiro[4,6-dihydropyrazolo[4,3-c]pyridine-7,3′-azetidin-1-ium]-5-carbonyl]indol-1-yl]-2-methyl-cyclopropyl]-4H-1,2,4-oxadiazol-5-one;
3-[(1S,2S)-1-[2-[(4S)-2-(4-fluoro-3,5-dimethyl-phenyl)-3-[3-(4-fluoro-1-methyl-indazol-5-yl)-2-oxo-imidazol-1-yl]-4-methyl-1′-(2,2,2-trifluoroethyl)spiro[4,6-dihydropyrazolo[4,3-c]pyridine-7,3′-azetidine]-5-carbonyl]-5-tetrahydropyran-4-yl-indol-1-yl]-2-methyl-cyclopropyl]-4H-1,2,4-oxadiazol-5-one;
3-[(1S,2S)-1-[2-[(4S)-1′-(2,2-difluoroethyl)-2-(4-fluoro-3,5-dimethyl-phenyl)-3-[3-(4-fluoro-1-methyl-indazol-5-yl)-2-oxo-imidazol-1-yl]-4-methyl-spiro[4,6-dihydropyrazolo[4,3-c]pyridine-7,3′-azetidin-1-ium]-5-carbonyl]-5-tetrahydropyran-4-yl-indol-1-yl]-2-methyl-cyclopropyl]-4H-1,2,4-oxadiazol-5-one;
3-[(1S,2S)-1-[2-[(4S)-2-(4-fluoro-3,5-dimethyl-phenyl)-3-[3-(4-fluoro-1-methyl-indazol-5-yl)-2-oxo-imidazol-1-yl]-1′-[2-[2-[2-(2-methoxyethoxy)ethoxy]ethoxy]ethyl]-4-methyl-spiro[4,6-dihydropyrazolo[4,3-c]pyridine-7,3′-azetidin-1-ium]-5-carbonyl]-5-tetrahydropyran-4-yl-indol-1-yl]-2-methyl-cyclopropyl]-4H-1,2,4-oxadiazol-5-one;
3-[(1S,2S)-1-[2-[(4S)-1-benzyl-2-(4-fluoro-3,5-dimethyl-phenyl)-3-[3-(4-fluoro-1-methyl-indazol-5-yl)-2-oxo-imidazol-1-yl]-4-methyl-spiro[4,6-dihydropyrazolo[4,3-c]pyridine-7,3′-azetidin-1-ium]-5-carbonyl]-5-tetrahydropyran-4-yl-indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[3-(4-fluoro-1-methyl-indazol-5-yl)-2-oxo-imidazol-1-yl]-1′-(2-hydroxy-2-methyl-propyl)-4-methyl-spiro[4,6-dihydropyrazolo[4,3-c]pyridine-7,3′-azetidin-1-ium]-5-carbonyl]indol-1-yl]-2-methyl-cyclopropyl]-4H-1,2,4-oxadiazol-5-one;
3-[(1S,2S)-1-[2-[(4S)-1′-cyclohexyl-2-(4-fluoro-3,5-dimethyl-phenyl)-3-[3-(4-fluoro-1-methyl-indazol-5-yl)-2-oxo-imidazol-1-yl]-4-methyl-spiro[4,6-dihydropyrazolo[4,3-c]pyridine-7,3′-azetidin-1-ium]-5-carbonyl]-5-tetrahydropyran-4-yl-indol-1-yl]-2-methyl-cyclopropyl]-4H-1,2,4-oxadiazol-5-one;
3-[(1S,2S)-1-[2-[(4S)-1′-(cyclopropylmethyl)-2-(4-fluoro-3,5-dimethyl-phenyl)-3-[3-(4-fluoro-1-methyl-indazol-5-yl)-2-oxo-imidazol-1-yl]-4-methyl-spiro[4,6-dihydropyrazolo[4,3-c]pyridine-7,3′-azetidin-1-ium]-5-carbonyl]-5-tetrahydropyran-4-yl-indol-1-yl]-2-methyl-cyclopropyl]-4H-1,2,4-oxadiazol-5-one;
3-[(1S,2S)-1-[2-[(4S)-2-(4-fluoro-3,5-dimethyl-phenyl)-3-[3-(4-fluoro-1-methyl-indazol-5-yl)-2-oxo-imidazol-1-yl]-4-methyl-1′-methylsulfonyl-spiro[4,6-dihydropyrazolo[4,3-c]pyridine-7,3′-azetidine]-5-carbonyl]-5-tetrahydropyran-4-yl-indol-1-yl]-2-methyl-cyclopropyl]-4H-1,2,4-oxadiazol-5-one;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-4-methyl-2-[5-(trifluoromethyl)-3-pyridyl]-6,7-dihydro-4H-pyrazolo[4,3-c]pyridin-3-yl]-N-(4-fluoro-1-methyl-indazol-5-yl)acetamide;
2-[(4S)-2-(3-cyclopropyl-4-fluoro-phenyl)-5-[7-[(4S)-2,2-dimethyltetrahydropyran-4-yl]-3-[1-(5-oxo-4H-1,2,4-oxadiazol-3-yl)cyclopropyl]indolizine-2-carbonyl]-4-methyl-6,7-dihydro-4H-pyrazolo[4,3-c]pyridin-3-yl]-N-(4-fluoro-1-methyl-indazol-5-yl)acetamide;
3-[1-[2-[(4S)-2-(3-cyclopropyl-4-fluoro-phenyl)-4-methyl-3-[1-(1-methylindazol-5-yl)-2-oxo-3-pyridyl]-6,7-dihydro-4H-pyrazolo[4,3-c]pyridine-5-carbonyl]-7-[(4S)-2,2-dimethyltetrahydropyran-4-yl]indolizin-3-yl]cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-4-methyl-3-[5-(3-methylimidazo[1,5-a]pyridin-7-yl)-1H-1,2,4-triazol-4-ium-3-yl]-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[5-(4-fluoro-1-methyl-indazol-5-yl)-1H-1,2,4-triazol-4-ium-3-yl]-4-methyl-4,6,7,8-tetrahydropyrazolo[4,3-c]azepine-5-carbonyl]indol-1-yl]-2-methyl-cyclopropyl]-4H-1,2,4-oxadiazol-5-one;
3-[(1S,2S)-1-[2-[(4S)-3-[5-(2,3-dimethylimidazo[1,2-a]pyridin-6-yl)-1H-1,2,4-triazol-4-ium-3-yl]-2-(4-fluoro-3,5-dimethyl-phenyl)-4-methyl-6,7-dihydro-4H-pyrazolo[4,3-c]pyridine-5-carbonyl]-5-[(4S)-2,2-dimethyltetrahydropyran-4-yl]indol-1-yl]-2-methyl-cyclopropyl]-4H-1,2,4-oxadiazol-5-one;
1-[(4S)-2-(4-fluoro-3,5-dimethyl-phenyl)-4-methyl-5-[1-[(1S,2S)-2-methyl-1-(5-oxo-4H-1,2,4-oxadiazol-3-yl)cyclopropyl]-5-tetrahydropyran-4-yl-indole-2-carbonyl]-6,7-dihydro-4H-pyrazolo[4,3-c]pyridin-3-yl]-N-(4-fluoro-1-methyl-indazol-5-yl)cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-1-ium-3-yl]-N-(1-methylindazol-5-yl)cyclopropanecarboxamide;
N-[2-(cyclopropoxy)pyridin-1-ium-4-yl]-1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]cyclopropanecarboxamide;
N-[4-(cyclopropoxy)cyclohexyl]-1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-1-ium-3-yl]cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-1-ium-3-yl]-N-(2-fluorophenyl)cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-N-(2-fluorophenyl)cyclopropanecarboxamide;
1-[(4S)-2-(4-fluoro-3,5-dimethyl-phenyl)-4-methyl-5-[5-[(2S)-2-methylmorpholin-4-ium-4-yl]-1-[(1S,2S)-2-methyl-1-(5-oxo-4H-1,2,4-oxadiazol-3-yl)cyclopropyl]indole-2-carbonyl]-6,7-dihydro-4H-pyrazolo[4,3-c]pyridin-3-yl]-N-(4-fluoro-1-methyl-indazol-5-yl)cyclopropanecarboxamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-1-ium-3-yl]-N-(4-fluoro-1-methyl-indazol-5-yl)propanamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-1-ium-3-yl]-N-(4-fluoro-1-methyl-indazol-5-yl)propanamide;
N-(1-methylindazol-5-yl)-2-[rac-(4S)-5-[5-(2,2-dimethyltetrahydropyran-4-yl)-1-[rac-(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]propanamide;
N-(1-methylindazol-5-yl)-2-[rac-(4S)-5-[5-(2,2-dimethyltetrahydropyran-4-yl)-1-[rac-(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]propanamide;
N-(2-fluorophenyl)-2-[rac-(4S)-5-[5-(2,2-dimethyltetrahydropyran-4-yl)-1-[rac-(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]propanamide;
N-(2-fluorophenyl)-2-[rac-(4S)-5-[5-(2,2-dimethyltetrahydropyran-4-yl)-1-[rac-(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]propanamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-1-ium-3-yl]-N-(4-methoxy-1-bicyclo[2.2.2]octanyl)propanamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-1-ium-3-yl]-N-(4-methoxy-1-bicyclo[2.2.2]octanyl)propanamide;
N-[4-(cyclopropoxy)cyclohexyl]-2-[rac-(4S)-5-[5-(2,2-dimethyltetrahydropyran-4-yl)-1-[rac-(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]propanamide;
N-[4-(cyclopropoxy)cyclohexyl]-2-[rac-(4S)-5-[5-(2,2-dimethyltetrahydropyran-4-yl)-1-[rac-(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]propanamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-3-yl]-N-(4-fluoro-1-methyl-indazol-5-yl)propanamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-3-yl]-N-(4-fluoro-1-methyl-indazol-5-yl)propanamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-1-ium-3-yl]-N-(4-fluoro-1-methyl-indazol-5-yl)cyclopropanecarboxamide;
N-(1,3-benzothiazol-3-ium-6-yl)-1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-3-yl]cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-1-ium-3-yl]-N-(2-methylindazol-5-yl)cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-1-ium-3-yl]-N-(1-methylindazol-5-yl)cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-1-ium-3-yl]-N-[1-(trideuteriomethyl)indazol-5-yl]cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-1-ium-3-yl]-N-(6-fluoro-1-methyl-indazol-5-yl)cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-1-ium-3-yl]-N-(1-ethylindazol-5-yl)cyclopropanecarboxamide;
N-(1-cyclopropyl-4-fluoro-indazol-5-yl)-1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-1-ium-3-yl]cyclopropanecarboxamide;
N-(1-cyclopropylindazol-5-yl)-1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-1-ium-3-yl]cyclopropanecarboxamide;
1-[(4S)-2-(4-fluoro-3,5-dimethyl-phenyl)-4-methyl-5-[1-[(1S,2S)-2-methyl-1-(5-oxo-4H-1,2,4-oxadiazol-3-yl)cyclopropyl]-5-tetrahydropyran-4-yl-indole-2-carbonyl]-4,6,7,8-tetrahydropyrazolo[4,3-c]azepin-1-ium-3-yl]-N-(4-fluoro-1-methyl-indazol-5-yl)cyclopropanecarboxamide;
N-(1-cyclopropyl-4-fluoro-indazol-5-yl)-1-[(4S)-2-(4-fluoro-3,5-dimethyl-phenyl)-4-methyl-5-[1-[(1S,2S)-2-methyl-1-(5-oxo-4H-1,2,4-oxadiazol-3-yl)cyclopropyl]-5-tetrahydropyran-4-yl-indole-2-carbonyl]-4,6,7,8-tetrahydropyrazolo[4,3-c]azepin-1-ium-3-yl]cyclopropanecarboxamide;
N-[4-(cyclopropoxy)cyclohexyl]-1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-1-ium-3-yl]cyclopropanecarboxamide;
N-(1-cyclopropyl-4-fluoro-indazol-5-yl)-1-[(4S)-2-(4-fluoro-3,5-dimethyl-phenyl)-4-methyl-5-[5-[(2S)-2-methylmorpholin-4-ium-4-yl]-1-[(1S,2S)-2-methyl-1-(5-oxo-4H-1,2,4-oxadiazol-3-yl)cyclopropyl]indole-2-carbonyl]-4,6,7,8-tetrahydropyrazolo[4,3-c]azepin-3-yl]cyclopropanecarboxamide;
1-[(4S)-2-(4-fluoro-3,5-dimethyl-phenyl)-4-methyl-5-[5-[(2S)-2-methylmorpholin-4-ium-4-yl]-1-[(1S,2S)-2-methyl-1-(5-oxo-4H-1,2,4-oxadiazol-3-yl)cyclopropyl]indole-2-carbonyl]-4,6,7,8-tetrahydropyrazolo[4,3-c]azepin-3-yl]-N-(4-fluoro-1-methyl-indazol-5-yl)cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-1-ium-3-yl]-N-[1-(2,2,2-trifluoroethyl)indazol-5-yl]cyclopropanecarboxamide;
N-(1-cyclopropyl-4-fluoro-indazol-5-yl)-1-[(4S)-2-(4-fluoro-3,5-dimethyl-phenyl)-4-methyl-5-[5-[(2S)-2-methylmorpholin-4-ium-4-yl]-1-[(1S,2S)-2-methyl-1-(5-oxo-4H-1,2,4-oxadiazol-3-yl)cyclopropyl]indole-2-carbonyl]spiro[6,8-dihydro-4H-pyrazolo[4,3-c]azepine-7,1′-cyclopropane]-3-yl]cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-3-yl]-N-isoquinolin-2-ium-6-yl-cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-3-yl]-N-(1-methylisoquinolin-2-ium-6-yl)cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-3-yl]-N-(5,6,7,8-tetrahydroquinolin-1-ium-6-yl)cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-3-yl]-N-(5,6,7,8-tetrahydroisoquinolin-7-yl)cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-3-yl]-N-(5,6,7,8-tetrahydroisoquinolin-7-yl)cyclopropanecarboxamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-N-(4-fluoro-1-methyl-indazol-5-yl)-2-hydroxy-propanamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-2-fluoro-N-(4-fluoro-1-methyl-indazol-5-yl)propanamide;
2-cyclopropyl-2-[(4S)-5-[5-[(4S)-2,2-dimethyltetrahydropyran-4-yl]-1-[(1 S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-N-(4-fluoro-1-methyl-indazol-2-ium-5-yl)-2-hydroxy-acetamide;
2-cyclopropyl-2-[(4S)-5-[5-[(4S)-2,2-dimethyltetrahydropyran-4-yl]-1-[(1 S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-N-(4-fluoro-1-methyl-indazol-2-ium-5-yl)-2-hydroxy-acetamide;
2-cyclopropyl-2-[(4S)-5-[5-[(4S)-2,2-dimethyltetrahydropyran-4-yl]-1-[(1 S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-2-fluoro-N-(4-fluoro-1-methyl-indazol-5-yl)acetamide;
2-cyclopropyl-2-[(4S)-5-[5-[(4S)-2,2-dimethyltetrahydropyran-4-yl]-1-[(1 S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-2-fluoro-N-(4-fluoro-1-methyl-indazol-5-yl)acetamide;
2-cyclopropyl-2-[(4S)-5-[5-[(4S)-2,2-dimethyltetrahydropyran-4-yl]-1-[(1 S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-2-fluoro-N-(1-methyl-6-isoquinolyl)acetamide;
2-cyclopropyl-2-[(4S)-5-[5-[(4S)-2,2-dimethyltetrahydropyran-4-yl]-1-[(1 S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-2-fluoro-N-(1-methyl-6-isoquinolyl)acetamide;
[1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-2-[(4-fluoro-1-methyl-indazol-2-ium-5-yl)amino]-1-methyl-2-oxo-ethyl]-dimethyl-ammonium;
[1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-2-[(4-fluoro-1-methyl-indazol-2-ium-5-yl)amino]-1-methyl-2-oxo-ethyl]-dimethyl-ammonium;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-N-(4-fluoro-1-methyl-indazol-5-yl)-2-methoxy-propanamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-N-(4-fluoro-1-methyl-indazol-5-yl)-2-methoxy-propanamide;
3-[(1S,2S)-1-[2-[(4S)-2-(1-cyclopropylpyrazol-4-yl)-3-[3-(4-fluoro-1-methyl-indazol-5-yl)-2-oxo-imidazol-1-yl]-4-methyl-spiro[4,6-dihydropyrazolo[4,3-c]pyridine-7,1′-cyclopropane]-5-carbonyl]-5-[(4S)-2,2-dimethyltetrahydropyran-4-yl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-3-[3-(4-fluoro-1-methyl-indazol-5-yl)-2-oxo-imidazol-1-yl]-4-methyl-2-[1-methyl-5-(trifluoromethyl)pyrazol-3-yl]-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-3-[3-(4-fluoro-1-methyl-indazol-5-yl)-2-oxo-imidazol-1-yl]-4-methyl-2-[1-methyl-5-(trifluoromethyl)pyrazol-3-yl]spiro[4,6-dihydropyrazolo[4,3-c]pyridine-7,1′-cyclopropane]-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-3-[3-(4-fluoro-1-methyl-indazol-5-yl)-2-oxo-imidazol-1-yl]-4-methyl-2-[5-(trifluoromethyl)-3-pyridyl]spiro[4,6-dihydropyrazolo[4,3-c]pyridine-7,1′-cyclopropane]-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-4-methyl-3-[5-(2-methyl-4-pyridyl)-1H-1,2,4-triazol-4-ium-3-yl]-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[3-(4-fluoro-1-methyl-indazol-5-yl)-2-oxo-3-azabicyclo[3.1.0]hexan-1-yl]-4-methyl-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[3-(4-fluoro-1-methyl-indazol-5-yl)-2-oxo-3-azabicyclo[3.1.0]hexan-1-yl]-4-methyl-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[5-(4-fluoro-1-methyl-indazol-2-ium-5-yl)-6-oxo-pyridazin-1-yl]-4-methyl-6,7-dihydro-4H-pyrazolo[4,3-c]pyridine-5-carbonyl]indol-1-yl]-2-methyl-cyclopropyl]-4H-1,2,4-oxadiazol-5-one;
1-[(4S)-5-[7-(2,2-dimethyltetrahydropyran-4-yl)-3-[1-(5-oxo-4H-1,2,4-oxadiazol-3-yl)cyclopropyl]indolizine-2-carbonyl]-2-(4-fluoro-3,5-dimethyl-phenyl)-4-methyl-6,7-dihydro-4H-pyrazolo[4,3-c]pyridin-3-yl]-N-(4-fluoro-1-methyl-indazol-5-yl)cyclopropanecarboxamide;
3-[1-[7-(2,2-dimethyltetrahydropyran-4-yl)-2-[(4S)-2-(4-fluoro-3,5-dimethyl-phenyl)-3-[1-(4-fluoro-1-methyl-indazol-5-yl)-2-oxo-pyrrolidin-3-yl]-4-methyl-6,7-dihydro-4H-pyrazolo[4,3-c]pyridine-5-carbonyl]indolizin-3-yl]cyclopropyl]-4H-1,2,4-oxadiazol-5-one;
3-[1-[7-(2,2-dimethyltetrahydropyran-4-yl)-2-[(4S)-2-(4-fluoro-3,5-dimethyl-phenyl)-3-[1-(4-fluoro-1-methyl-indazol-5-yl)-2-oxo-pyrrolidin-3-yl]-4-methyl-6,7-dihydro-4H-pyrazolo[4,3-c]pyridine-5-carbonyl]indolizin-3-yl]cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[5-(4-fluoro-1-methyl-indazol-5-yl)-6-oxo-pyrimidin-1-yl]-4-methyl-6,7-dihydro-4H-pyrazolo[4,3-c]pyridin-1-ium-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[5-(4-fluoro-1-methyl-indazol-5-yl)-6-oxo-pyrimidin-1-yl]-4-methyl-4,6,7,8-tetrahydropyrazolo[4,3-c]azepine-5-carbonyl]indol-1-yl]-2-methyl-cyclopropyl]-4H-1,2,4-oxadiazol-5-one;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-1-ium-3-yl]-4-(4-fluoro-1-methyl-indazol-5-yl)-2-methyl-morpholin-3-one;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-1-ium-3-yl]-4-(4-fluoro-1-methyl-indazol-5-yl)-2-methyl-morpholin-3-one;
3-[(1S,2S)-1-[5-[(4S)-2,2-dimethyltetrahydropyran-4-yl]-2-[(4S)-2-(4-fluoro-3,5-dimethyl-phenyl)-4-methyl-3-[4-(1-methylindazol-2-ium-5-yl)-5-oxo-1,2,4-triazol-1-yl]-4,6,7,8-tetrahydropyrazolo[4,3-c]azepine-5-carbonyl]indol-1-yl]-2-methyl-cyclopropyl]-4H-1,2,4-oxadiazol-5-one;
3-[(1S,2S)-1-[2-[(4S)-2-(3-cyclopropyl-4-fluoro-phenyl)-3-[4-(4-fluoro-1-methyl-indazol-2-ium-5-yl)-5-oxo-1,2,4-triazol-1-yl]-4-methyl-4,6,7,8-tetrahydropyrazolo[4,3-c]azepine-5-carbonyl]-5-[(4S)-2,2-dimethyltetrahydropyran-4-yl]indol-1-yl]-2-methyl-cyclopropyl]-4H-1,2,4-oxadiazol-5-one;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-N-(4-fluoro-1-methyl-indazol-2-ium-5-yl)-2-methyl-propanamide;
3-[(1S,2S)-1-[2-[(4S)-2-(4-fluoro-3,5-dimethyl-phenyl)-3-[3-(4-fluoro-1-methyl-indazol-5-yl)-2-oxo-imidazol-1-yl]-4-methyl-spiro[4,6-dihydropyrazolo[4,3-c]pyridine-7,3′-azetidine]-5-carbonyl]-5-tetrahydropyran-4-yl-indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-4-ethyl-2-(4-fluoro-3,5-dimethyl-phenyl)-3-[3-(4-fluoro-1-methyl-indazol-5-yl)-2-oxo-imidazol-1-yl]-4,6,7,8-tetrahydropyrazolo[4,3-c]azepin-1-ium-5-carbonyl]indol-1-yl]-2-methyl-cyclopropyl]-4H-1,2,4-oxadiazol-5-one;
3-[(1S,2S)-1-[5-(2,2-dimethyltetrahydropyran-4-yl)-2-[(4S)-2-(4-fluoro-3,5-dimethyl-phenyl)-3-[1-(4-fluoro-1-methyl-indazol-5-yl)-2-oxo-pyrrolidin-3-yl]-4-methyl-6,7-dihydro-4H-pyrazolo[4,3-c]pyridin-1-ium-5-carbonyl]indol-1-yl]-2-methyl-cyclopropyl]-4H-1,2,4-oxadiazol-5-one;
3-[(1S,2S)-1-[5-(2,2-dimethyltetrahydropyran-4-yl)-2-[(4S)-2-(4-fluoro-3,5-dimethyl-phenyl)-3-[1-(4-fluoro-1-methyl-indazol-5-yl)-2-oxo-pyrrolidin-3-yl]-4-methyl-6,7-dihydro-4H-pyrazolo[4,3-c]pyridin-1-ium-5-carbonyl]indol-1-yl]-2-methyl-cyclopropyl]-4H-1,2,4-oxadiazol-5-one;
3-[(1S,2S)-1-[2-[(4S)-3-[2-[2-(cyclopropoxy)-4-pyridyl]-3-oxo-pyridazin-4-yl]-2-(4-fluoro-3,5-dimethyl-phenyl)-4-methyl-6,7-dihydro-4H-pyrazolo[4,3-c]pyridine-5-carbonyl]-5-[(4S)-2,2-dimethyltetrahydropyran-4-yl]indol-1-yl]-2-methyl-cyclopropyl]-4H-1,2,4-oxadiazol-5-one;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-4-methyl-2-[5-(trifluoromethyl)-3-pyridyl]-6,7-dihydro-4H-pyrazolo[4,3-c]pyridin-3-yl]-N-(4-fluoro-1-methyl-indazol-5-yl)propanamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-4-methyl-2-[5-(trifluoromethyl)-3-pyridyl]-6,7-dihydro-4H-pyrazolo[4,3-c]pyridin-3-yl]-N-(4-fluoro-1-methyl-indazol-5-yl)propanamide;
N-(4-fluoro-1-methyl-indazol-5-yl)-2-[(4S)-4,7,7-trimethyl-5-[5-[(2S)-2-methylmorpholin-4-yl]-1-[(1S,2S)-2-methyl-1-(5-oxo-4H-1,2,4-oxadiazol-3-yl)cyclopropyl]indole-2-carbonyl]-2-[5-(trifluoromethyl)pyridin-1-ium-3-yl]-4,6-dihydropyrazolo[4,3-c]pyridin-3-yl]propanamide;
N-(4-fluoro-1-methyl-indazol-5-yl)-2-[(4S)-4,7,7-trimethyl-5-[5-[(2S)-2-methylmorpholin-4-yl]-1-[(1S,2S)-2-methyl-1-(5-oxo-4H-1,2,4-oxadiazol-3-yl)cyclopropyl]indole-2-carbonyl]-2-[5-(trifluoromethyl)pyridin-1-ium-3-yl]-4,6-dihydropyrazolo[4,3-c]pyridin-3-yl]propanamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-1′-(2,2,2-trifluoroethyl)spiro[4,6-dihydropyrazolo[4,3-c]pyridine-7,3′-azetidin-1-ium]-3-yl]-N-(4-fluoro-1-methyl-indazol-5-yl)cyclopropanecarboxamide;
3-[(1S,2S)-1-[2-[(4S)-2-(5-cyclopropylpyridin-1-ium-3-yl)-3-[3-(4-fluoro-1-methyl-indazol-5-yl)-2-oxo-imidazol-1-yl]-4-methyl-6,7-dihydro-4H-pyrazolo[4,3-c]pyridine-5-carbonyl]-5-[(4S)-2,2-dimethyltetrahydropyran-4-yl]indol-1-yl]-2-methyl-cyclopropyl]-4H-1,2,4-oxadiazol-5-one;
3-[(1S,2S)-1-[2-[(4S)-3-[1-[4-(cyclopropoxy)cyclohexyl]-6-oxo-pyrimidin-5-yl]-2-(4-fluoro-3,5-dimethyl-phenyl)-4-methyl-4,6,7,8-tetrahydropyrazolo[4,3-c]azepin-1-ium-5-carbonyl]-5-[(4S)-2,2-dimethyltetrahydropyran-4-yl]indol-1-yl]-2-methyl-cyclopropyl]-4H-1,2,4-oxadiazol-5-one;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4,7,7-trimethyl-6,8-dihydro-4H-pyrazolo[4,3-c]azepin-1-ium-3-yl]-N-(4-fluoro-1-methyl-indazol-5-yl)acetamide;
1-[(4S)-1′-(2,2-difluoroethyl)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-spiro[4,6-dihydropyrazolo[4,3-c]pyridine-7,3′-azetidin-1-ium]-3-yl]-N-(4-fluoro-1-methyl-indazol-5-yl)cyclopropanecarboxamide;
3-[(1S,2S)-1-[2-[(4S)-2-(4-fluoro-3,5-dimethyl-phenyl)-4-methyl-3-[2-(1-methylindazol-5-yl)-3-oxo-pyridazin-4-yl]spiro[4,6-dihydropyrazolo[4,3-c]pyridine-7,1′-cyclopropane]-5-carbonyl]-5-[(2S)-2-methylmorpholin-4-ium-4-yl]indol-1-yl]-2-methyl-cyclopropyl]-4H-1,2,4-oxadiazol-5-one;
3-[(1S,2S)-1-[2-[(4S)-3-[1-[2-(difluoromethyl)indazol-5-yl]-2-oxo-3-pyridyl]-2-(5-fluoro-4,6-dimethyl-pyridin-1-ium-2-yl)-4-methyl-6,7-dihydro-4H-pyrazolo[4,3-c]pyridine-5-carbonyl]-5-[(4S)-2,2-dimethyltetrahydropyran-4-yl]indol-1-yl]-2-methyl-cyclopropyl]-4H-1,2,4-oxadiazol-5-one;
3-[(1S,2S)-1-[2-[(4S)-2-(4-fluoro-3,5-dimethyl-phenyl)-3-[2-(4-fluoro-1-methyl-indazol-5-yl)-3-oxo-pyridazin-4-yl]-4-methyl-6,7-dihydro-4H-pyrazolo[4,3-c]pyridine-5-carbonyl]-5-[(2S)-2-methylmorpholin-4-ium-4-yl]indol-1-yl]-2-methyl-cyclopropyl]-4H-1,2,4-oxadiazol-5-one;
3-[(1S,2S)-1-[5-(2,2-difluoromorpholin-4-ium-4-yl)-2-[(4S)-2-(4-fluoro-3,5-dimethyl-phenyl)-3-[1-(4-fluoro-1-methyl-indazol-5-yl)-2-oxo-3-pyridyl]-4-methyl-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[2-[(4S)-2-(4-fluoro-3,5-dimethyl-phenyl)-3-[1-(4-fluoro-1-methyl-indazol-5-yl)-2-oxo-3-pyridyl]-4-methyl-6,7-dihydro-4H-pyrazolo[4,3-c]pyridine-5-carbonyl]-5-morpholin-4-ium-4-yl-indol-1-yl]-2-methyl-cyclopropyl]-4H-1,2,4-oxadiazol-5-one;
3-[(1S,2S)-1-[2-[(4S)-2-(4-fluoro-3,5-dimethyl-phenyl)-3-[1-(4-fluoro-1-methyl-indazol-5-yl)-6-oxo-pyrimidin-5-yl]-4-methyl-6,7-dihydro-4H-pyrazolo[4,3-c]pyridine-5-carbonyl]-5-[(2S)-2-methylmorpholin-4-ium-4-yl]indol-1-yl]-2-methyl-cyclopropyl]-4H-1,2,4-oxadiazol-5-one;
3-[(1S,2S)-1-[2-[(4S)-2-(4-fluoro-3,5-dimethyl-phenyl)-3-[1-(4-fluoro-1-methyl-indazol-5-yl)-6-oxo-pyrimidin-5-yl]-4-methyl-6,7-dihydro-4H-pyrazolo[4,3-c]pyridine-5-carbonyl]-5-morpholin-4-ium-4-yl-indol-1-yl]-2-methyl-cyclopropyl]-4H-1,2,4-oxadiazol-5-one;
3-[(1S,2S)-1-[2-[(4S)-2-(4-fluoro-3,5-dimethyl-phenyl)-3-[1-(4-fluoro-1-methyl-indazol-5-yl)-2-oxo-3-pyridyl]-4-methyl-6,7-dihydro-4H-pyrazolo[4,3-c]pyridine-5-carbonyl]-5-[(2S)-2-methylmorpholin-4-ium-4-yl]indol-1-yl]-2-methyl-cyclopropyl]-4H-1,2,4-oxadiazol-5-one;
3-[(1S,2S)-1-[2-[(4S)-2-(4-fluoro-3,5-dimethyl-phenyl)-4,7,7-trimethyl-3-[1-(1-methylindazol-5-yl)-2-oxo-3-pyridyl]-4,6-dihydropyrazolo[4,3-c]pyridine-5-carbonyl]-5-[(2S)-2-methylmorpholin-4-ium-4-yl]indol-1-yl]-2-methyl-cyclopropyl]-4H-1,2,4-oxadiazol-5-one;
3-[(1S,2S)-1-[2-[(4S)-3-[1-[2-(cyclopropoxy)-4-pyridyl]-2-oxo-3-pyridyl]-2-(5-fluoro-4,6-dimethyl-pyridin-1-ium-2-yl)-4-methyl-6,7-dihydro-4H-pyrazolo[4,3-c]pyridine-5-carbonyl]-5-[(4S)-2,2-dimethyltetrahydropyran-4-yl]indol-1-yl]-2-methyl-cyclopropyl]-4H-1,2,4-oxadiazol-5-one;
3-[(1S,2S)-1-[2-[(4S)-2-(4-fluoro-3,5-dimethyl-phenyl)-3-[(1S,6S)-3-(4-fluoro-1-methyl-indazol-5-yl)-2-oxo-3-azabicyclo[4.1.0]heptan-1-yl]-4-methyl-6,7-dihydro-4H-pyrazolo[4,3-c]pyridine-5-carbonyl]-5-[(2S)-2-methylmorpholin-4-ium-4-yl]indol-1-yl]-2-methyl-cyclopropyl]-4H-1,2,4-oxadiazol-5-one,
2-[(4S)-2-(4-fluoro-3,5-dimethyl-phenyl)-4-methyl-5-[1-[(1S,2S)-2-methyl-1-(5-oxo-4H-1,2,4-oxadiazol-3-yl)cyclopropyl]-5-tetrahydropyran-4-yl-indole-2-carbonyl]-6,7-dihydro-4H-pyrazolo[4,3-c]pyridin-3-yl]-N-(4-fluoro-1-methyl-indazol-2-ium-5-yl)-2-methyl-propanamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-3-yl]-2-fluoro-N-(1-methyl-6-isoquinolyl)propanamide;
3-[(1S,2S)-1-[5-[(4S)-2,2-dimethyltetrahydropyran-4-yl]-2-[(4S)-2-(4-fluoro-3,5-dimethyl-phenyl)-3-[3-fluoro-1-(4-fluoro-1-methyl-indazol-2-ium-5-yl)-2-oxo-pyrrolidin-3-yl]-4-methyl-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[3-fluoro-1-(4-fluoro-1-methyl-indazol-2-ium-5-yl)-2-oxo-pyrrolidin-3-yl]-4-methyl-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[3-(4-fluoro-1-methyl-indazol-2-ium-5-yl)-2-oxo-3-azabicyclo[3.1.0]hexan-1-yl]-4-methyl-4,6,7,8-tetrahydropyrazolo[4,3-c]azepine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-4-methyl-3-[3-(2-methylquinolin-1-ium-6-yl)-2-oxo-3-azabicyclo[3.1.0]hexan-1-yl]-6,7-dihydro-4H-pyrazolo[4,3-c]pyridine-5-carbonyl]indol-1-yl]-2-methyl-cyclopropyl]-4H-1,2,4-oxadiazol-5-one;
2-[(4S)-2-(4-fluoro-3,5-dimethyl-phenyl)-4,7,7-trimethyl-5-[1-[(1S,2S)-2-methyl-1-(5-oxo-4H-1,2,4-oxadiazol-3-yl)cyclopropyl]-5-tetrahydropyran-4-yl-indole-2-carbonyl]-4,6-dihydropyrazolo[4,3-c]pyridin-3-yl]-N-(4-fluoro-1-methyl-indazol-5-yl)-2-methyl-propanamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4,7,7-trimethyl-4,6-dihydropyrazolo[4,3-c]pyridin-3-yl]-N-(4-fluoro-1-methyl-indazol-5-yl)-2-methyl-propanamide;
3-[(1R,2S)-1-[5-(2,2-dimethyltetrahydropyran-4-yl)-2-[(4S)-2-(4-fluoro-3,5-dimethyl-phenyl)-3-[3-(4-fluoro-1-methyl-indazol-5-yl)-2-oxo-imidazol-1-yl]-4-methyl-6,7-dihydro-4H-pyrazolo[4,3-c]pyridin-1-ium-5-carbonyl]phenyl]-2-methyl-cyclopropyl]-4H-1,2,4-oxadiazol-5-one;
2-[(4S)-2-(4-fluoro-3,5-dimethyl-phenyl)-4-methyl-5-[2-[(1S,2S)-2-methyl-1-(5-oxo-4H-1,2,4-oxadiazol-3-yl)cyclopropyl]-5-(1,3,7-trimethylindazol-6-yl)pyrazole-3-carbonyl]-6,7-dihydro-4H-pyrazolo[4,3-c]pyridin-3-yl]-N-(4-fluoro-1-methyl-indazol-5-yl)acetamide;
N-(1-cyclopropyl-4-fluoro-indazol-5-yl)-1-[(4S)-5-[5-(3,3-dimethyl-2-oxo-indolin-5-yl)-2-[(1S,2S)-2-methyl-1-(5-oxo-4H-1,2,4-oxadiazol-3-yl)cyclopropyl]pyrazole-3-carbonyl]-2-(4-fluoro-3,5-dimethyl-phenyl)-4-methyl-6,7-dihydro-4H-pyrazolo[4,3-c]pyridin-3-yl]cyclopropanecarboxamide;
N-(1-cyclopropyl-4-fluoro-indazol-5-yl)-1-[(4S)-2-(4-fluoro-3,5-dimethyl-phenyl)-4-methyl-5-[2-[(1S,2S)-2-methyl-1-(5-oxo-4H-1,2,4-oxadiazol-3-yl)cyclopropyl]-5-[1-methyl-3-(trifluoromethyl)pyrazol-5-yl]pyrazole-3-carbonyl]-6,7-dihydro-4H-pyrazolo[4,3-c]pyridin-3-yl]cyclopropanecarboxamide;
1-[(4S)-5-[5-(1,7-dimethylindazol-6-yl)-2-[(1S,2S)-2-methyl-1-(5-oxo-4H-1,2,4-oxadiazol-3-yl)cyclopropyl]pyrazole-3-carbonyl]-2-(4-fluoro-3,5-dimethyl-phenyl)-4-methyl-6,7-dihydro-4H-pyrazolo[4,3-c]pyridin-3-yl]-N-(4-fluoro-1-methyl-indazol-5-yl)cyclopropanecarboxamide;
N-(1-cyclopropyl-4-fluoro-indazol-5-yl)-1-[(4S)-2-(4-fluoro-3,5-dimethyl-phenyl)-4-methyl-5-[5-[4-(3-methyloxetan-3-yl)phenyl]-2-[(1S,2S)-2-methyl-1-(5-oxo-4H-1,2,4-oxadiazol-3-yl)cyclopropyl]pyrazole-3-carbonyl]-6,7-dihydro-4H-pyrazolo[4,3-c]pyridin-3-yl]cyclopropanecarboxamide;
1-[(4S)-5-[5-(1,7-dimethylindazol-6-yl)-2-[(1S,2S)-2-methyl-1-(5-oxo-4H-1,2,4-oxadiazol-3-yl)cyclopropyl]pyrazole-3-carbonyl]-2-(4-fluoro-3,5-dimethyl-phenyl)-4-methyl-spiro[4,6-dihydropyrazolo[4,3-c]pyridine-7,1′-cyclobutane]-3-yl]-N-(4-fluoro-1-methyl-indazol-5-yl)cyclopropanecarboxamide;
N-(1-cyclopropyl-4-fluoro-indazol-5-yl)-1-[(4S)-5-[5-(1,7-dimethylindazol-6-yl)-2-[(1S,2S)-2-methyl-1-(5-oxo-4H-1,2,4-oxadiazol-3-yl)cyclopropyl]pyrazole-3-carbonyl]-2-(4-fluoro-3,5-dimethyl-phenyl)-4-methyl-6,7-dihydro-4H-pyrazolo[4,3-c]pyridin-3-yl]cyclopropanecarboxamide;
N-(1-cyclopropyl-4-fluoro-indazol-5-yl)-1-[(4S)-2-(4-fluoro-3,5-dimethyl-phenyl)-5-[5-[4-(1-methoxycyclobutyl)phenyl]-2-[(1S,2S)-2-methyl-1-(5-oxo-4H-1,2,4-oxadiazol-3-yl)cyclopropyl]pyrazole-3-carbonyl]-4-methyl-6,7-dihydro-4H-pyrazolo[4,3-c]pyridin-3-yl]cyclopropanecarboxamide;
3-[(1S,2S)-1-[5-[(4S)-2-(4-fluoro-3,5-dimethyl-phenyl)-3-[3-(4-fluoro-1-methyl-indazol-5-yl)-2-oxo-imidazol-1-yl]-4-methyl-6,7-dihydro-4H-pyrazolo[4,3-c]pyridine-5-carbonyl]-3-(5-methyl-6-isoquinolyl)pyrazol-1-yl]-2-methyl-cyclopropyl]-4H-1,2,4-oxadiazol-5-one;
5-[4-[(4S)-2-(4-fluoro-3,5-dimethyl-phenyl)-3-[3-(4-fluoro-1-methyl-indazol-2-ium-5-yl)-2-oxo-imidazol-1-yl]-4-methyl-6,7-dihydro-4H-pyrazolo[4,3-c]pyridin-1-ium-5-carbonyl]-3-[(1R,2S)-1-(5-hydroxy-1,2,4-oxadiazole-2,4-diium-3-yl)-2-methyl-cyclopropyl]phenyl]-3,3-dimethyl-indolin-2-one;
N-(2,3-dimethyl-4-pyridyl)-2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-3-yl]acetamide;
N-(1-cyclopropylindazol-5-yl)-2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-spiro[6,8-dihydro-4H-pyrazolo[4,3-c]azepin-1-ium-7,1′-cyclopropane]-3-yl]acetamide;
N-(1-cyclopropylindazol-2-ium-5-yl)-2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4,7,7-trimethyl-6,8-dihydro-4H-pyrazolo[4,3-c]azepin-3-yl]acetamide;
N-(1-cyclopropylindazol-5-yl)-2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-spiro[4,6-dihydropyrazolo[4,3-c]pyridine-7,1′-cyclobutane]-3-yl]acetamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-spiro[6,8-dihydro-4H-pyrazolo[4,3-c]azepin-1-ium-7,1′-cyclopropane]-3-yl]-N-(4-fluoro-1-methyl-indazol-5-yl)acetamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-spiro[6,8-dihydro-4H-pyrazolo[4,3-c]azepine-7,1′-cyclopropane]-3-yl]-N-(1-methylisoquinolin-2-ium-6-yl)acetamide;
N-(1-cyclopropyl-4-fluoro-indazol-5-yl)-2-[(4S)-2-(4-fluoro-3,5-dimethyl-phenyl)-4-methyl-5-[1-[(1S,2S)-2-methyl-1-(5-oxo-4H-1,2,4-oxadiazol-3-yl)cyclopropyl]-5-tetrahydropyran-4-yl-indole-2-carbonyl]spiro[4,6-dihydropyrazolo[4,3-c]pyridine-7,1′-cyclobutane]-3-yl]acetamide;
N-(4,6-difluoro-1-methyl-indazol-5-yl)-2-[(4S)-2-(4-fluoro-3,5-dimethyl-phenyl)-4-methyl-5-[1-[(1S,2S)-2-methyl-1-(5-oxo-4H-1,2,4-oxadiazol-3-yl)cyclopropyl]-5-tetrahydropyran-4-yl-indole-2-carbonyl]spiro[4,6-dihydropyrazolo[4,3-c]pyridine-7,1′-cyclobutane]-3-yl]acetamide;
N-(1-cyclopropyl-4-fluoro-indazol-5-yl)-2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-spiro[4,6-dihydropyrazolo[4,3-c]pyridine-7,1′-cyclobutane]-3-yl]acetamide;
N-(4,6-difluoro-1-methyl-indazol-5-yl)-2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-spiro[6,8-dihydro-4H-pyrazolo[4,3-c]azepin-1-ium-7,1′-cyclopropane]-3-yl]acetamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-spiro[6,8-dihydro-4H-pyrazolo[4,3-c]azepine-7,1′-cyclopropane]-3-yl]-N-(3-methylisoquinolin-2-ium-6-yl)acetamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-spiro[6,8-dihydro-4H-pyrazolo[4,3-c]azepine-7,1′-cyclopropane]-3-yl]-N-(2-methylquinolin-1-ium-6-yl)acetamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4,7,7-trimethyl-6,8-dihydro-4H-pyrazolo[4,3-c]azepin-1-ium-3-yl]-N-(4-fluoro-1-methyl-indazol-5-yl)acetamide;
3-[(1S,2S)-1-[2-[(4S)-2-(4-fluoro-3,5-dimethyl-phenyl)-4-methyl-3-[3-(1-methylisoquinolin-2-ium-6-yl)-2-oxo-3-azabicyclo[4.1.0]heptan-1-yl]-6,7-dihydro-4H-pyrazolo[4,3-c]pyridine-5-carbonyl]-5-[(2S)-2-methylmorpholin-4-yl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[3-(7-fluoro-1-methyl-isoquinolin-2-ium-6-yl)-2-oxo-3-azabicyclo[4.1.0]heptan-1-yl]-4-methyl-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[3-(5-fluoro-1-methyl-isoquinolin-2-ium-6-yl)-2-oxo-3-azabicyclo[4.1.0]heptan-1-yl]-4-methyl-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[3-(7-fluoro-2-methyl-quinolin-1-ium-6-yl)-2-oxo-3-azabicyclo[4.1.0]heptan-1-yl]-4-methyl-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[2-[(4S)-3-[3-(1-cyclopropyl-4-fluoro-indazol-2-ium-5-yl)-2-oxo-3-azabicyclo[4.1.0]heptan-1-yl]-2-(4-fluoro-3,5-dimethyl-phenyl)-4-methyl-6,7-dihydro-4H-pyrazolo[4,3-c]pyridine-5-carbonyl]-5-[(4S)-2,2-dimethyltetrahydropyran-4-yl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[(1S,6S)-3-(4-hydroxycyclohexyl)-2-oxo-3-azabicyclo[4.1.0]heptan-1-yl]-4-methyl-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[3-(6-fluoro-1-methyl-indazol-2-ium-5-yl)-2-oxo-3-azabicyclo[4.1.0]heptan-1-yl]-4-methyl-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[2-[(4S)-3-[(1S,6S)-3-[4-(cyclopropoxy)cyclohexyl]-2-oxo-3-azabicyclo[4.1.0]heptan-1-yl]-2-(4-fluoro-3,5-dimethyl-phenyl)-4-methyl-6,7-dihydro-4H-pyrazolo[4,3-c]pyridine-5-carbonyl]-5-[(4S)-2,2-dimethyltetrahydropyran-4-yl]indol-1-yl]-2-methyl-cyclopropyl]-4H-1,2,4-oxadiazol-5-one
N-(4-chloro-1-methyl-indazol-5-yl)-1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-1-ium-3-yl]cyclopropanecarboxamide;
1-[(4S)-2-(4-fluoro-3,5-dimethyl-phenyl)-4-methyl-5-[1-[(1S,2S)-2-methyl-1-(5-oxo-4H-1,2,4-oxadiazol-3-yl)cyclopropyl]-5-tetrahydropyran-4-yl-indole-2-carbonyl]spiro[4,6-dihydropyrazolo[4,3-c]pyridine-7,1′-cyclobutane]-3-yl]-N-(1-methylisoquinolin-2-ium-6-yl)cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-1-ium-3-yl]-N-(6-fluoro-1-methyl-indazol-5-yl)cyclopropanecarboxamide;
1-[(4S)-2-(4-fluoro-3,5-dimethyl-phenyl)-4-methyl-5-[1-[(1S,2S)-2-methyl-1-(5-oxo-4H-1,2,4-oxadiazol-3-yl)cyclopropyl]-5-tetrahydropyran-4-yl-indole-2-carbonyl]-6,7-dihydro-4H-pyrazolo[4,3-c]pyridin-1-ium-3-yl]-N-(6-fluoro-1-methyl-indazol-5-yl)cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-N-(5,6,7,8-tetrahydroisoquinolin-2-ium-7-yl)cyclopropanecarboxamide;
N-(4,6-difluoro-1-methyl-indazol-5-yl)-1-[(4S)-2-(4-fluoro-3,5-dimethyl-phenyl)-4-methyl-5-[1-[(1S,2S)-2-methyl-1-(5-oxo-4H-1,2,4-oxadiazol-3-yl)cyclopropyl]-5-tetrahydropyran-4-yl-indole-2-carbonyl]-6,7-dihydro-4H-pyrazolo[4,3-c]pyridin-1-ium-3-yl]cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-N-(5,6,7,8-tetrahydroisoquinolin-2-ium-7-yl)cyclopropanecarboxamide;
N-(1-cyclopropyl-4-fluoro-indazol-5-yl)-1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-1-ium-3-yl]cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-N-(2-methylquinolin-1-ium-6-yl)cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4,7,7-trimethyl-4,6-dihydropyrazolo[4,3-c]pyridin-1-ium-3-yl]-N-(1-methylindazol-5-yl)cyclopropanecarboxamide;
1-[(4S)-2-(4-fluoro-3,5-dimethyl-phenyl)-4-methyl-5-[1-[(1S,2S)-2-methyl-1-(5-oxo-4H-1,2,4-oxadiazol-3-yl)cyclopropyl]-5-tetrahydropyran-4-yl-indole-2-carbonyl]spiro[4,6-dihydropyrazolo[4,3-c]pyridin-1-ium-7,1′-cyclobutane]-3-yl]-N-(1-methylindazol-5-yl)cyclopropanecarboxamide;
N-(4,6-difluoro-1-methyl-indazol-5-yl)-1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-1-ium-3-yl]cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4,7,7-trimethyl-4,6-dihydropyrazolo[4,3-c]pyridin-1-ium-3-yl]-N-(4-fluoro-1-methyl-indazol-5-yl)cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-1-ium-3-yl]-N-(7-fluoro-2-methyl-indazol-5-yl)cyclopropanecarboxamide;
N-(4,6-difluoro-1-methyl-indazol-5-yl)-1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-1-ium-3-yl]cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-spiro[4,6-dihydropyrazolo[4,3-c]pyridin-1-ium-7,1′-cyclobutane]-3-yl]-N-(4-fluoro-1-methyl-indazol-5-yl)cyclopropanecarboxamide;
N-(1-cyclopropyl-4-fluoro-indazol-5-yl)-1-[(4S)-2-(4-fluoro-3,5-dimethyl-phenyl)-4-methyl-5-[2-[(1S,2S)-2-methyl-1-(5-oxo-4H-1,2,4-oxadiazol-3-yl)cyclopropyl]-5-(4-morpholinophenyl)pyrazole-3-carbonyl]-6,7-dihydro-4H-pyrazolo[4,3-c]pyridin-3-yl]cyclopropanecarboxamide;
N-(4-chloro-1-methyl-indazol-5-yl)-1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-1-ium-3-yl]cyclopropanecarboxamide;
1-[(4S)-2-(4-fluoro-3,5-dimethyl-phenyl)-4-methyl-5-[5-[(2S)-2-methylmorpholin-4-yl]-1-[(1S,2S)-2-methyl-1-(5-oxo-4H-1,2,4-oxadiazol-3-yl)cyclopropyl]indole-2-carbonyl]spiro[4,6-dihydropyrazolo[4,3-c]pyridine-7,1′-cyclobutane]-3-yl]-N-(1-methylisoquinolin-2-ium-6-yl)cyclopropanecarboxamide;
N-(1-cyclopropyl-4-fluoro-indazol-2-ium-5-yl)-2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4,7,7-trimethyl-4,6-dihydropyrazolo[4,3-c]pyridin-3-yl]-2-hydroxy-propanamide;
2-cyclopropyl-2-[(4S)-5-[5-[(4S)-2,2-dimethyltetrahydropyran-4-yl]-1-[(1 S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-2-fluoro-N-(1-methylindazol-5-yl)acetamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-1′-[(1-methylcyclopropyl)methyl]spiro[4,6-dihydropyrazolo[4,3-c]pyridine-7,3′-azetidin-1-ium]-3-yl]-N-(4-fluoro-1-methyl-indazol-5-yl)cyclopropanecarboxamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-spiro[4,6-dihydropyrazolo[4,3-c]pyridine-7,1′-cyclobutane]-3-yl]-N-(5-fluoro-1-methyl-6-isoquinolyl)acetamide;
N-(1-cyclopropyl-4-fluoro-indazol-5-yl)-1-[(4S)-5-[4-(3,3-dimethyl-2-oxo-indolin-5-yl)-2-[(1R,2S)-2-methyl-1-(5-oxo-4H-1,2,4-oxadiazol-3-yl)cyclopropyl]benzoyl]-2-(4-fluoro-3,5-dimethyl-phenyl)-4-methyl-6,7-dihydro-4H-pyrazolo[4,3-c]pyridin-3-yl]cyclopropanecarboxamide;
N-(1-cyclopropyl-4-fluoro-indazol-5-yl)-1-[(4S)-2-(4-fluoro-3,5-dimethyl-phenyl)-5-[4-isochroman-6-yl-2-[(1R,2S)-2-methyl-1-(5-oxo-4H-1,2,4-oxadiazol-3-yl)cyclopropyl]benzoyl]-4-methyl-6,7-dihydro-4H-pyrazolo[4,3-c]pyridin-3-yl]cyclopropanecarboxamide;
N-(1-cyclopropyl-4-fluoro-indazol-5-yl)-1-[(4S)-2-(4-fluoro-3,5-dimethyl-phenyl)-5-[4-(6-isoquinolyl)-2-[(1R,2S)-2-methyl-1-(5-oxo-4H-1,2,4-oxadiazol-3-yl)cyclopropyl]benzoyl]-4-methyl-6,7-dihydro-4H-pyrazolo[4,3-c]pyridin-3-yl]cyclopropanecarboxamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-spiro[4,6-dihydropyrazolo[4,3-c]pyridine-7,1′-cyclobutane]-3-yl]-N-(7-fluoro-1-methyl-6-isoquinolyl)acetamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4,7,7-trimethyl-4,6-dihydropyrazolo[4,3-c]pyridin-3-yl]-N-(5-fluoro-2-methyl-6-quinolyl)acetamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4,7,7-trimethyl-4,6-dihydropyrazolo[4,3-c]pyridin-3-yl]-N-(5-fluoro-1-methyl-6-isoquinolyl)acetamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-spiro[4,6-dihydropyrazolo[4,3-c]pyridine-7,1′-cyclobutane]-3-yl]-N-(5-fluoro-2-methyl-6-quinolyl)acetamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-N-(5-fluoro-2-methyl-quinolin-1-ium-6-yl)cyclopropanecarboxamide; and
3-[(1S,2S)-1-[5-[(4S)-2,2-dimethyltetrahydropyran-4-yl]-2-[(4S)-2-(4-fluoro-3,5-dimethyl-phenyl)-3-[3-(5-fluoro-2-methyl-quinolin-1-ium-6-yl)-2-oxo-3-azabicyclo[4.1.0]heptan-1-yl]-4-methyl-6,7-dihydro-4H-pyrazolo[4,3-c]pyridine-5-carbonyl]indol-1-yl]-2-methyl-cyclopropyl]-4H-1,2,4-oxadiazol-5-one,
   or a pharmaceutically acceptable salt thereof

In embodiment no. 136, the present invention provides a compound of Formula I as set forth in any one of embodiment nos. 1-132, or a pharmaceutically acceptable salt thereof, wherein Y is other than

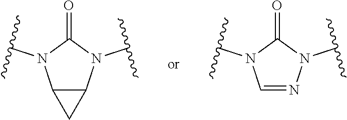 [SMILES: *N1C(=O)N(*)C2CC21 ; *n1cnn(*)c1=O]

wherein the left [symbol] is bound to the pyrazole moiety and the right [symbol] is bound to B.

In embodiment no. 137, the present invention provides a compound of Formula I as set forth in any one of embodiment nos. 1-132 and 136, or a pharmaceutically acceptable salt thereof, wherein Y is other than

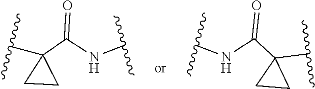 [SMILES: *NC(=O)C1(*)CC1 ; *NC(=O)C1(*)CC1]

wherein the left [symbol] on Y is bound to the pyrazole moiety and the right [symbol] on Y is bound to B.

In embodiment no. 138, the present invention provides a compound of Formula I as set forth in any one of embodiment nos. 1-132 and 136-129, or a pharmaceutically acceptable salt thereof, wherein Y is other than

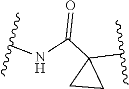 [SMILES: *NC(=O)C1(*)CC1]

wherein the left [symbol] on Y is bound to the pyrazole moiety and the right [symbol] on Y is bound to B.

In embodiment no. 139, the present invention provides a compound of Formula I as set forth in any one of embodiment nos. 1-132 and 136-138, or a pharmaceutically acceptable salt thereof, wherein Y is

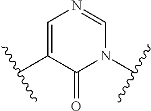 [SMILES: *c1cncn(*)c1=O]

### and B is

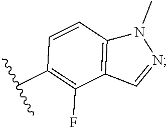 [SMILES: *c1ccc2c(cnn2C)c1F]

or Y is other than

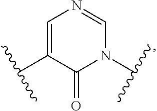 [SMILES: *c1cncn(*)c1=O]

wherein the left [symbol] N is bound to the pyrazole moiety and the right [symbol] is bound to B;

In embodiment no. 140, the present invention provides a compound of Formula I as set forth in any one of embodiment nos. 1-132 and 136-139, or a pharmaceutically acceptable salt thereof, wherein Y is other than

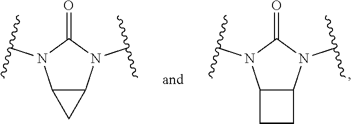 [SMILES: *N1C(=O)N(*)C2CC21 ; *N1C(=O)N(*)C2CCC21]

wherein the left [symbol] on Y is bound to the pyrazole moiety and the right [symbol] on Y is bound to B.

In embodiment no. 141, the present invention provides a compound of Formula I as set forth in any one of embodiment nos. 1-132 and 136-140, or a pharmaceutically acceptable salt thereof, wherein Y is other than

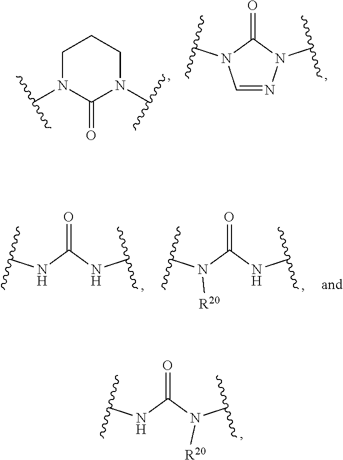

wherein R²⁰ is selected from C₁-C₃ alkyl or C₁-C₃ cycloalkyl, wherein the left on Y is bound to the pyrazole moiety and the right [symbol] on Y is bound to B.

In embodiment no. 142, the present invention provides a compound of Formula I as set forth in any one of embodiment nos. 1-132 and 136-141, or a pharmaceutically acceptable salt thereof, wherein Y is other than

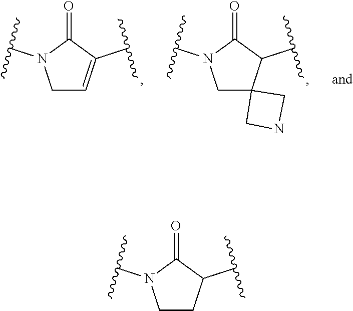 [SMILES: *C1=CCN(*)C1=O ; *C1C(=O)N(*)CC12CNC2 ; *C1CCN(*)C1=O]

wherein the left [symbol] on Y is bound to the pyrazole moiety and the right [symbol] on Y is bound to B.

In embodiment no. 143, the present invention provides a compound of Formula I as set forth in any one of embodiment nos. 1-132 and 136-142, or a pharmaceutically acceptable salt thereof, wherein Y is other than

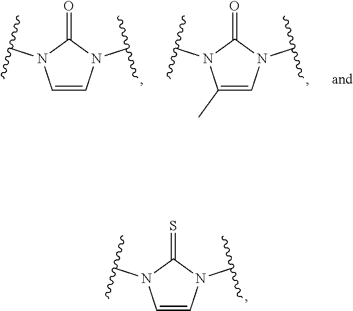

wherein the left [symbol] on Y is bound to the pyrazole moiety and the right [symbol] on Y is bound to B.

In instances, the pyrazole moiety is referred to as the pyrazolopyridine structure or the pyrazole.

The present disclosure is also directed to a pharmaceutical composition which comprises a compound according to any one of embodiments 1-135, or a pharmaceutically acceptable salt thereof, and a pharmaceutically acceptable carrier.

The present disclosure is also directed to a method for treating obesity or non-insulin-dependent diabetes mellitus (Type 2 diabetes) which comprises administering to a subject in need of such treatment (i) a therapeutically effective amount of a compound according to any one of embodiments 1-135, or a pharmaceutically acceptable salt thereof, or (ii) a pharmaceutical composition which comprises a compound according to any one of embodiments 1-135, or a pharmaceutically acceptable salt thereof, and a pharmaceutically acceptable carrier.

The present disclosure is also directed to use of a compound according to any one of embodiments 1-135, or a pharmaceutically acceptable salt thereof, in the manufacture of a medicament for treating obesity or non-insulin-dependent diabetes mellitus (Type 2 diabetes).

### Definitions

Listed below are definitions of various terms used herein. These definitions apply to the terms as they are used throughout this specification and claims, unless otherwise limited in specific instances, either individually or as part of a larger group.

Unless defined otherwise, all technical and scientific terms used herein have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” in quantitative terms refers to plus or minus 10% of the value it modifies (rounded up to the nearest whole number if the value is not sub-dividable, such as a number of molecules or nucleotides).

All ranges disclosed herein are inclusive of the recited endpoint and independently combinable (for example, the range of “from 50 mg to 500 mg” is inclusive of the endpoints, 50 mg and 500 mg, and all the intermediate values). The endpoints of the ranges and any values disclosed herein are not limited to the precise range or value; they are sufficiently imprecise to include values approximating these ranges and/or values.

As used herein, the term “comprising” may include the embodiments “consisting of” and “consisting essentially of” The terms “comprise(s),” “include(s),” “having,” “has,” “may,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated components, which allows the presence of only the named components or compounds, along with any acceptable carriers or fluids, and excludes other components or compounds.

With respect to a compound of the invention which has one or more asymmetric centers and can occur as mixtures of stereoisomers, a substantially pure compound can be either a substantially pure mixture of the stereoisomers or a substantially pure individual diastereomer or enantiomer unless expressly depicted otherwise. The present invention encompasses all stereoisomeric forms of the compounds of Formula I. Unless a specific stereochemistry is indicated, the present invention is meant to comprehend all such isomeric forms of these compounds. Centers of asymmetry that are present in the compounds of Formula I can all independently of one another have (R) configuration or (S) configuration. When bonds to the chiral carbon are depicted as straight lines in the structural Formulas of the invention, it is understood that both the (R) and (S) configurations of the chiral carbon, and hence both enantiomers and mixtures thereof, are embraced within the Formula. Similarly, when a compound name is recited without a chiral designation for a chiral carbon, it is understood that both the (R) and (S) configurations of the chiral carbon, and hence individual enantiomers, diastereomers and mixtures thereof, are embraced by the name. The production of specific stereoisomers or mixtures thereof may be identified in the Examples where such stereoisomers or mixtures were obtained, but this in no way limits the inclusion of all stereoisomers and mixtures thereof from being within the scope of this invention.

The invention includes all possible enantiomers and diastereomers and mixtures of two or more stereoisomers, for example mixtures of enantiomers and/or diastereomers, in all ratios. Thus, enantiomers are a subject of the invention in enantiomerically pure form, both as levorotatory and as dextrorotatory antipodes, in the form of racemates and in the form of mixtures of the two enantiomers in all ratios. In the case of a cis/trans isomerism the invention includes both the cis form and the trans form as well as mixtures of these forms in all ratios. The preparation of individual stereoisomers can be carried out, if desired, by separation of a mixture by customary methods, for example by chromatography or crystallization, by the use of stereochemically uniform starting materials for the synthesis or by stereoselective synthesis. Optionally a derivatization can be carried out before a separation of stereoisomers. The separation of a mixture of stereoisomers can be carried out at an intermediate step during the synthesis of a compound of Formula I or it can be done on a final racemic product. Absolute stereochemistry may be determined by X-ray crystallography of crystalline products or crystalline intermediates which are derivatized, if necessary, with a reagent containing a stereogenic center of known configuration. Unless a particular isomer, salt, solvate (including hydrates) or solvated salt of such racemate, enantiomer, or diastereomer is indicated, the present invention includes all such isomers, as well as salts, solvates (including hydrates) and solvated salts of such racemates, enantiomers, diastereomers and mixtures thereof.

The term “alkyl”, as well as other groups having the prefix “alk”, such as alkoxy, dialkylamino, and trialkylammonium, and the like, refers to an aliphatic hydrocarbon group having one of its hydrogen atoms replaced with a bond. An alkyl group may be straight or branched and contain from about 1 to about 10 carbon atoms. In one embodiment, an alkyl group contains from about 1 to about 10 carbon atoms. In different embodiments, an alkyl group contains from 1 to 6 carbon atoms (C₁₋₆ alkyl) or from about 1 to about 4 carbon atoms (C₁-C₄ alkyl). Non-limiting examples of alkyl groups include methyl, ethyl, n-propyl, isopropyl, n-butyl, sec-butyl, isobutyl, tert-butyl, n-pentyl, neopentyl, isopentyl, n-hexyl, isohexyl and neohexyl. In one embodiment, an alkyl group is linear. In another embodiment, an alkyl group is branched. Unless otherwise indicated, an alkyl group (and any other group recited herein) is unsubstituted.

“Alkylene” or “alkylenyl” refers to an alkyl group, as defined above, wherein one of the alkyl group's hydrogen atoms has been replaced with a bond. The alkylene diradicals are also known as alkylenyl radicals. Non-limiting examples of alkylene groups include —CH₂—, —CH₂CH₂—, —CH₂CH₂CH₂—, —CH₂CH₂CH₂CH₂—, —CH(CH₃)CH₂CH₂—, —CH(CH₃)— and —CH₂CH(CH₃)CH₂—. In one embodiment, an alkylene group has from 1 to about 10 carbon atoms. In another embodiment, an alkylene group has from 1 to about 6 carbon atoms. In another embodiment, an alkylene group is branched. In another embodiment, an alkylene group is linear. In one embodiment, an alkylene group is —CH₂—. The term “C₁-C₆ alkylene” refers to an alkylene group having from 1 to 6 carbon atoms.

“Alkynylene,” and “alkynylenyl” as used herein, refers to an alkynyl group, as defined above, wherein one of the alkynyl group's hydrogen atoms has been replaced with a bond. Non-limiting examples of alkylene groups include —C≡C—, —C≡CCH₂—, and —C≡CCH(CH₃)₂—. In one embodiment, an alkynylene group has from 2 to about 6 carbon atoms. In another embodiment, an alkynylene group has from 2 to about 10 carbon atoms. In another embodiment, an alkynylene group is branched. In another embodiment, an alkynylene group is linear. The term “C₂-C₆ alkynylene” refers to an alkynylene group having from 2 to 6 carbon atoms. The term “C₂-C₁₀ alkynylene” refers to an alkynylene group having from 2 to 10 carbon atoms.

“Alkoxy”, “alkyloxy” and “alkyl-O—” are used interchangeably and refer to an alkyl (carbon and hydrogen chain) group linked to oxygen (R—O). Non-limiting examples of alkoxy are methoxy (CH₃O—), ethoxy (CH₃CH₂O—) and propoxy (CH₃CH₂CH₂O—).

“Aminoalkyl” means saturated carbon chains which may be linear or branched or combinations thereof which are substituted with one amino group which may be terminal (—NH₂) or internal (—NH—).

“Hydroxyalkyl” means saturated carbon chains which may be linear or branched or combinations thereof which are substituted with one hydroxy (—OH) group.

“Diaminoalkyl” means saturated carbon chains which may be linear or branched or combinations thereof which are substituted with two amino (—NH₂) groups.

“Dihydroxyalkyl” means saturated carbon chains which may be linear or branched or combinations thereof which are substituted with two hydroxy (—OH) groups.

“Hydroxyaminoalkyl” means saturated carbon chains which may be linear or branched or combinations thereof which are substituted with one hydroxy (—OH) group and one amino (—NH₂) group.

“Aryl” refers to an aromatic monocyclic or multicyclic ring moiety comprising 5 to 14 ring carbon atoms, or more specifically, 6 to 10 ring carbon atoms. Monocyclic aryl rings include, but are not limited to, phenyl and naphthyl. Bonding can be through any of the carbon atoms of any ring.

“Cycloalkyl” (or “C₃₋₁₂ cycloalkyl”) means any univalent non-aromatic radical derived from a monocyclic, bicyclic, tricyclic or tetracyclic ring system having 3 to 12 ring carbons atoms. These non-aromatic radicals, which have 3, 4, 5, 6, 7, 8, or up to 12 carbon ring atoms may be fully saturated, or partially unsaturated. Unless stated specifically in the specification, the cycloalkyl radical may be a monocyclic, bicyclic, tricyclic or tetracyclic ring system, which may include fused or bridged ring systems. Here, the point of attachment for a “cycloalkyl” to the rest of the molecule is on the saturated ring. Bicyclic cycloalkyl ring systems include fused ring systems, where two rings share two atoms (e.g., decalin), spiro ring systems where two rings share one atom (e.g., spiro[4.5]decanyl) and bridged groups (e.g., norbornyl).

Additional examples within the above meaning include, but are not limited to univalent radicals of cyclopropyl, cyclobutyl, cyclopentyl, cyclohexyl, cycloheptyl, bicyclo[2.2.2]octanyl, bicyclo[1.1.1]pentanyl, bicyclo[2.2.1]heptanyl, [1.1.1]-bicyclo pentane, bicyclo[3.1.0]hexanyl, cyclohexenyl, cyclopentenyl, 1-decalinyl, spiro[2.4]heptyl, spiro[2.3]hexyl, spiro[2.2]pentyl, and norbornyl.

The term “C₃₋₈ cycloalkyl” (or “C₃-C₈ cycloalkyl” or “C₃₋₈ cycloalkyl”) means a cyclic ring of an alkane having three to eight total carbon atoms (i.e., cyclopropyl, cyclobutyl, cyclopentyl, cyclohexyl, cycloheptyl, or cyclooctyl). The terms “C₃₋₇ cycloalkyl”, “C₃₋₆ cycloalkyl”, “C₅₋₇ cycloalkyl” and the like have analogous meanings.

“Cycloheteroalkyl” or “heterocycloalkyl” means a stable and non-aromatic (including not fully aromatic, e.g., one double bond) monocyclic, bicyclic (including spirocyclic) or bridged carbocyclic ring or ring system comprising 3 to about 12 ring atoms, containing at least one ring heteroatom selected from N, S and O and the remainder of the ring atoms are carbon atoms. The nitrogen or sulfur atom of the heterocycloalkyl can be optionally oxidized to the corresponding N-oxide, S-oxide (S═O) or S-dioxide (SO₂). Where the ring or ring system contains one or more N atoms, the N can be in the form of quarternary amine. A heterocycloalkyl group can be joined via a ring carbon, or ring nitrogen atom, unless specified otherwise. The cycloheteroalkyl ring may be substituted on the ring carbons and/or the ring nitrogen(s). Whenever it appears herein, a numerical range such as “3 to 12” or “3-12” refers to each integer in the given range. In one embodiment, a heterocycloalkyl group is monocyclic and has from about 3 to about 7 ring atoms (a “3 to 7-membered monocyclic heterocycloalkyl” group). In another embodiment, a heterocycloalkyl group is monocyclic has from about 4 to about 7 ring atoms (a “4 to 7-membered monocyclic heterocycloalkyl” group). In other embodiments, the heterocycloalkyl group is bicyclic and has 7-10 ring atoms, 8-10 ring atoms, or 9 or 10 ring atoms (a “9 or 10-membered bicyclic heterocycloalkyl” group). In still another embodiment, a heterocycloalkyl group is monocyclic and has 5 or 6 ring atoms. In one embodiment, a heterocycloalkyl group is monocyclic. There are no adjacent oxygen and/or sulfur atoms present in the ring system. In some embodiments, the cycloheteroalkyl or the heterocycloalkyl is a tricyclic or tetracyclic ring system which are optionally spirocyclic containing at least one spiro atom.

Non-limiting examples of heterocycloalkyl rings include decahydroisoquinoline, dioxaspiro[4.5]decane, 2,5-diazabicyclo[2.2.1]heptyl, quinuclidinyl, oxetanyl, piperidyl, pyrrolidinyl, piperazinyl, morpholinyl, thiomorpholinyl, thiazolidinyl, 1,4-dioxanyl, tetrahydrofuranyl, tetrahydrothiophenyl, beta-lactam, gamma-lactam, delta-lactam, beta-lactone, gamma-lactone, delta-lactone, piperidinyl, 3-azabixyclo[3.1.0]hexyl, 2-azabicyclo[2.1.1]hexyl, 6-azaspiro[2.5]octanyl, azetidinyl, 2,3-dihydro-1H-indenyl, dihydro-1H-indenyl, 3H-spiro[benzofuran-2′,4′-piperidinyl, 2,3-dihydro-1H-pyrrolo[3,2,1-ij][1,6]naphthyridinyl, 3,4,6,7-tetrahydro-5H-imidazo[4,5-c]pyridyl, 3a,5,6,6a-tetrahydro-4H-pyrrolo[3,4-d]isoxazole, diazabicyclo[3.3.2]decanyl, 2,3,4,5,6,7-hexahydroisothiazolo[5,4-c]pyridyl, hexahydro-2H-pyrrolo[3,4-d]isothiazolyl, 3,9-diazabicyclo[3.3.2]decanyl, bicyclo[2,2,1]heptenyl, 2′,3′-dihydro-1′H-spiro[piperidine-4,4′-quinazolin], octahydropyrrolo[3,4-b][1,4]oxazinyl, (diazabicyclo[2.2.1]heptanyl), 2,5-diazabicyclo[2.2.1]heptanyl, tetrahydrobenzo[d]thiazolyl, 4,5,6,7-tetrahydrobenzo[d]thiazolyl, 2,3-dihydrobenzofuranyl, oxabicyclo[2.1.1]hexyl, dihydro-5H-pyrrolo[3,4-d]thiazolyl, 4,6-dihydro-5H-pyrrolo[3,4-d]thiazolyl, dihydro-5H-pyrrolo[3,4-d]oxazolyl, 4,6-dihydro-5H-pyrrolo[3,4-d]oxazolyl, dihydrothiazolo[5,4-c]pyridin-5(4H)-yl, 6,7-dihydrothiazolo[5,4-c]pyridin-5(4H)-yl, benzo[d]imidazolyl, 1H-enzo[d]imidazolyl, diazaspiro[4.4]nonanyl, and 2,7-diazaspiro[4.4]nonanyl, and pyrrolidinone, and oxides thereof and all isomers thereof.

“Fluoroalkyl” includes mono-substituted as well as multiple fluoro-substituted linear and branched alkyl groups, up to perfluoro substituted alkyl. “C₁₋₆ fluoroalkyl” refers to a fluoronated alkyl group containing from 1 to 6 carbon atoms. For example, fluoromethyl, 1,1-difluoroethyl, difluoromethyl, trifluoromethyl or 3,3,4,4,4-pentafluorobutyl are included.

“Heteroalkyl” means a non-cyclic stable straight or branched chain, or combination thereof, including at least one carbon atom and at least one heteroatom selected from O, N, P, Si, and S, (including S(O), S(O)₂, NH, and N(alkyl)), wherein the heteroatoms may be placed at any interior position of the heteroalkyl group or at the position at which the heteroalkyl group is attached to the remainder of the molecule. Non-limiting examples include ethers, thioethers, amines, and the like. Other non-limiting examples include alkoxy, and aminoalkyl. Further non-limiting examples include —OC₁-C₆ alkyl, —(C₁-C₆ alkyl)-OC₁-C₆ alkyl, —(C₁-C₆ alkyl)-O—(C₁-C₆ alkyl)-OC₁-C₆ alkyl, —NC₁-C₆ alkyl, —(C₁-C₆ alkyl)-NC₁-C₆ alkyl, and —(C₁-C₆ alkyl)-N—(C₁-C₆ alkyl)-NC₁-C₆ alkyl. Additional non-limiting examples include —OCF₂CF₃, —CH₂CH₂—O—CH₂CH₂—O—CH₃, —CH₂CH₂—O—CH₃, and —OCH₃.

“Heteroaryl” refers to an aromatic monocyclic or multicyclic ring moiety comprising ring carbon atoms and 1-4 ring heteroatoms provided in the aromatic ring system, wherein each heteroatom is independently selected from S, N, or O. Heteroaryl bicyclic ring systems can include one or more heteroatoms in one or both rings. Examples of heteroaryl rings include pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, and pyrazinyl. Other examples of heteroaryl rings include indolyl, indazolyl, and isoquinolinyl.

“Halogen” or “halo” includes fluorine, chlorine, bromine and iodine.

“Oxo” means an oxygen atom connected to another atom by a double bond and is represented by “═O” herein.

Where any amine is present in the compound, the N atom may be optionally in the form of a quaternary amine having one or more appropriate additional substitutions, as further described herein.

When any ring atom is specified as being optionally substituted with, or in a specified form, for example, S substituted with oxo groups, or N in the form of a N-oxide, this does not preclude the substitution of any ring atom with the other listed optional substituents when not substituted with oxo groups or in the form of a N-oxide.

When any variable (e.g., n, R^{a}, R^{b}, etc.) occurs more than one time in any constituent or in Formula I (or any other Formula), its definition on each occurrence is independent of its definition at every other occurrence. Also, combinations of substituents and/or variables are permissible only if such combinations result in stable compounds.

A wavy line [symbol] , as used herein, indicates a point of attachment to the rest of the compound. Lines drawn into a ring system, for example:

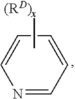

indicate that the bond may be attached to any of the substitutable ring atoms.

Under standard nomenclature used throughout this disclosure, the terminal portion of the designated side chain is described last, preceded by the adjacent functionality toward the point of attachment.

In choosing compounds of the present invention, one of ordinary skill in the art will recognize that the various substituents, R¹, R^{A}, etc., are to be chosen in conformity with well-known principles of chemical structure connectivity and stability.

The term “substituted” shall be deemed to include multiple degrees of substitution by a named substitutent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

In the compounds of Formula I, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of Formula I. For example, different isotopic forms of hydrogen (H) include protium (¹H) and deuterium (²H or D). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within Formula I, can be prepared without undue experimentation by conventional techniques well known to those skilled in the art or by processes analogous to those described in the Schemes and Examples herein using appropriate isotopically-enriched reagents and/or intermediates.

Unless expressly stated to the contrary in a particular context, any of the various cyclic ring and ring system variables or substituents described herein may be attached to the rest of the compound at any ring atom (i.e., any carbon atom or any heteroatom) provided that a stable compound results.

Unless expressly stated to the contrary, all ranges cited herein are inclusive. For example, a heteroaryl ring described as containing from “1 to 4 heteroatoms” means the ring can contain 1, 2, 3 or 4 heteroatoms. It is also to be understood that any range cited herein includes within its scope all of the sub-ranges within that range. Thus, for example, a heterocycloalkyl ring described as containing from “1 to 4 heteroatoms” is intended to include as aspects thereof, heterocycloalkyl rings containing 2 to 4 heteroatoms, 3 or 4 heteroatoms, 1 to 3 heteroatoms, 2 or 3 heteroatoms, 1 or 2 heteroatoms, 1 heteroatom, 2 heteroatoms, 3 heteroatoms, and 4 heteroatoms. Similarly, C₁-C₆ when used with a chain, for example an alkyl chain, means that the chain can contain 1, 2, 3, 4, 5 or 6 carbon atoms. It also includes all ranges contained therein including C₁-C₅, C₁-C₄, C₁-C₃, C₁-C₂, C₂-C₆, C₃-C₆, C₄-C₆, C₅-C₆, and all other possible combinations.

A “stable” compound is a compound which can be prepared and isolated and whose structure and properties remain or can be caused to remain essentially unchanged for a period of time sufficient to allow use of the compound for the purposes described herein (e.g., therapeutic administration to a subject). The compounds of the present invention are limited to stable compounds embraced by Formulas I.

The term “compound” refers to the compound and, in certain embodiments, to the extent they are stable, any hydrate or solvate thereof. A hydrate is the compound complexed with water, and a solvate is the compound complexed with an organic solvent.

As indicated above, the compounds of the present invention can be employed in the form of pharmaceutically acceptable salts. Those skilled in the art will recognize those instances in which the compounds of the invention may form salts. The term “pharmaceutically acceptable salt” refers to a salt (including an inner salt such as a zwitterion) which possesses effectiveness similar to the parent compound and which is not biologically or otherwise undesirable (e.g., is neither toxic nor otherwise deleterious to the recipient thereof). Thus, an embodiment of the invention provides pharmaceutically acceptable salts of the compounds of the invention. The term “salt(s)”, as employed herein, denotes any of the following: acidic salts formed with inorganic and/or organic acids, as well as basic salts formed with inorganic and/or organic bases. Salts of compounds of the invention may be formed by methods known to those of ordinary skill in the art, for example, by reacting a compound of the invention with an amount of acid or base, such as an equivalent amount, in a medium such as one in which the salt precipitates or in aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates (“mesylates”), naphthalenesulfonates, nitrates, oxalates, phosphates, propionates, salicylates, succinates, sulfates, tartarates, thiocyanates, toluenesulfonates (also known as tosylates) and the like. Additionally, acids which are generally considered suitable for the formation of pharmaceutically useful salts from basic pharmaceutical compounds are discussed, for example, by P. Stahl et al., et al., Camille G. (eds.) Handbook of Pharmaceutical Salts. Properties, Selection and Use. (2002) Zurich: Wiley-VCH; S. Berge et al., et al., Journal of Pharmaceutical Sciences (1977) 66(1) 1-19; P. Gould, International J. of Pharmaceutics (1986) 33 201-217; Anderson et al., et al., The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamine, t-butyl amine, choline, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

In addition, when a compound of the invention contains both a basic moiety, such as, but not limited to an aliphatic primary, secondary, tertiary or cyclic amine, an aromatic or heteroaryl amine, pyridine or imidazole, and an acidic moiety, such as, but not limited to tetrazole or carboxylic acid, zwitterions (“inner salts”) may be formed and are included within the terms “salt(s)” as used herein. It is understood that certain compounds of the invention may exist in zwitterionic form, having both anionic and cationic centers within the same compound and a net neutral charge. Such zwitterions are included within the invention.

The compounds of Formula I may exist as rapidly interconverting tautomers with different points of attachment of hydrogen accompanied by one or more double bond shifts. The individual tautomers as well as mixtures thereof are encompassed by the present invention. The ratio between the tautomeric forms will vary depending on the conditions. As is well known to one of ordinary skill in the art, such compounds may be drawn and named in different ways. For example, the following structures depicted below show different ways that an illustrative compound of the invention may be drawn:

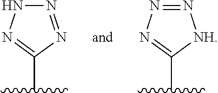 [SMILES: *c1nn[nH]n1 ; "and" ; *c1nnn[nH]1]

Furthermore, a subject of the present disclosure are pharmaceutical preparations (or pharmaceutical compositions) which comprise as active component a therapeutically effective dose of at least one compound of Formula I and/or a pharmaceutically acceptable salt thereof and a customary pharmaceutically acceptable carrier, i.e., one or more pharmaceutically acceptable carrier substances and/or additives.

Thus, a subject of the invention is, for example, said compound and its pharmaceutically acceptable salts for use as a pharmaceutical, pharmaceutical preparations which comprise as active component a therapeutically effective dose of said compound and/or a pharmaceutically acceptable salt thereof and a customary pharmaceutically acceptable carrier, and the uses of said compound and/or a pharmaceutically acceptable salt thereof in the therapy or prophylaxis of the above mentioned syndromes as well as their use for preparing medicaments for these purposes.

The pharmaceuticals according to the invention can be administered orally, for example in the form of pills, tablets, lacquered tablets, sugar-coated tablets, granules, hard and soft gelatin capsules, aqueous, alcoholic or oily solutions, syrups, emulsions or suspensions, or rectally, for example in the form of suppositories. Administration can also be carried out parenterally, for example subcutaneously, intramuscularly or intravenously in the form of solutions for injection or infusion. Other suitable administration forms are, for example, percutaneous or topical administration, for example in the form of ointments, tinctures, sprays or transdermal therapeutic systems, or the inhalative administration in the form of nasal sprays or aerosol mixtures, or, for example, microcapsules, implants or rods. The preferred administration form depends, for example, on the disease to be treated and on its severity.

For the production of pills, tablets, sugar-coated tablets and hard gelatin capsules it is possible to use, for example, lactose, starch, for example maize starch, or starch derivatives, talc, stearic acid or its salts, etc. Carriers for soft gelatin capsules and suppositories are, for example, fats, waxes, semisolid and liquid polyols, natural or hardened oils, etc. Suitable carriers for the preparation of solutions, for example of solutions for injection, or of emulsions or syrups are, for example, water, physiologically sodium chloride solution, alcohols such as ethanol, glycerol, polyols, sucrose, invert sugar, glucose, mannitol, vegetable oils, etc. It is also possible to lyophilize the compounds of Formula I and their pharmaceutically acceptable salts and to use the resulting lyophilizates, for example, for preparing preparations for injection or infusion. Suitable carriers for microcapsules, implants or rods are, for example, copolymers of glycolic acid and lactic acid.

Suitable solid or galenical preparation forms are, for example, granules, powders, coated tablets, tablets, capsules, microcapsules, suppositories, syrups, juices, suspensions, emulsions, drops or injectable solutions and preparations having prolonged release of active substance, in whose preparation customary excipients such as vehicles, disintegrants, binders, coating agents, swelling agents, glidants or lubricants, flavorings, sweeteners and solubilizers are used. Frequently used auxiliaries which may be mentioned are magnesium carbonate, titanium dioxide, lactose, mannitol and other sugars, talc, lactose, gelatin, starch, cellulose and its derivatives, animal and plant oils such as cod liver oil, sunflower, peanut or sesame oil, polyethylene glycol and solvents such as, for example, sterile water and mono- or polyhydric alcohols such as glycerol.

Besides the active compounds and carriers, the pharmaceutical preparations can also contain customary additives, for example fillers, disintegrants, binders, lubricants, wetting agents, stabilizers, emulsifiers, dispersants, preservatives, sweeteners, colorants, flavorings, aromatizers, thickeners, diluents, buffer substances, solvents, solubilizers, agents for achieving a depot effect, salts for altering the osmotic pressure, coating agents or antioxidants.

The compounds of the present disclosure can be administered alone or in combination with one or more additional therapeutic agents disclosed herein or other suitable agents, depending on the condition being treated. Hence, in some embodiments the one or more compounds of the invention will be co-administered with other agents as described herein. When used in combination therapy, the compounds described herein are administered with the second agent simultaneously or separately. This administration in combination can include simultaneous administration of the two agents in the same dosage form, simultaneous administration in separate dosage forms, and separate administration. That is, a compound of Formula I and any of the agents described herein can be formulated together in the same dosage form and administered simultaneously. Alternatively, a compound of Formula I and any of the agents described herein can be simultaneously administered, wherein both the agents are present in separate formulations. In another alternative, a compound of Formula I can be administered just followed by any of the agents described herein, or vice versa. In some embodiments of the separate administration protocol, a compound of Formula I and any of the agents described herein are administered a few minutes apart, or a few hours apart, or a few days apart.

As one aspect of the present disclosure contemplates the treatment of the disease/conditions with a combination of pharmaceutically active compounds that may be administered separately, the invention further relates to combining separate pharmaceutical compositions in kit form. The kit comprises two separate pharmaceutical compositions: a compound of Formula I, and a second pharmaceutical compound. The kit comprises a container for containing the separate compositions such as a divided bottle or a divided foil packet. Additional examples of containers include syringes, boxes, and bags. In some embodiments, the kit comprises directions for the use of the separate components. The kit form is particularly advantageous when the separate components are preferably administered in different dosage forms (e.g., oral, parenteral; IV, transdermal and subcutaneous), are administered at different dosage intervals, or when titration of the individual components of the combination is desired by the prescribing health care professional.

One or more additional pharmacologically active agents may be administered in combination with a compound of Formula I. An additional active agent (or agents) is intended to mean a pharmaceutically active agent (or agents) that is active in the body, including pro-drugs that convert to pharmaceutically active form after administration, which are different from the compound of Formula I and also includes free-acid, free-base and pharmaceutically acceptable salts of said additional active agents. Generally, any suitable additional active agent or agents, including but not limited to anti-hypertensive agents, anti-obetic, anti-inflammatory, anti-fibrotic, and anti-atherosclerotic agents such as a lipid modifying compound, anti-diabetic agents and/or anti-obesity agents may be used in any combination with the compound of Formula I in a single dosage formulation (a fixed dose drug combination), or may be administered to the patient in one or more separate dosage formulations which allows for concurrent or sequential administration of the active agents (co-administration of the separate active agents).

Examples of additional active agents which may be employed include but are not limited to anti-myostatin or anti-promyostatin antibodies (e. g. trevogrumab, apitegromab), anti-ACVR2/ACVR2B antibodies (e. g. bimagrumab), angiotensin converting enzyme inhibitors (e.g., alacepril, benazepril, captopril, ceronapril, cilazapril, delapril, enalapril, enalaprilat, fosinopril, imidapril, lisinopril, moveltipril, perindopril, quinapril, ramipril, spirapril, temocapril, or trandolapril), angiotensin II receptor antagonists (e.g., losartan, valsartan, candesartan, olmesartan, telmesartan and any of these drugs used in combination with hydrochlorothiazide such as HYZAAR®); inhibin/active βC or βE antibody or siRNA (e.g., WVE-007), apelin receptor antagonist (e. g. ANPA-0073), DGAT2 inhibitor (e.g., those disclosed in US2024-0327391 and WO2022/076496, each of which is hereby incorporated by reference), neutral endopeptidase inhibitors (e.g., thiorphan and phosphoramidon), aldosterone antagonists, aldosterone synthase inhibitors, renin inhibitors (e.g., urea derivatives of di- and tri-peptides, amino acids and derivatives, amino acid chains linked by non-peptidic bonds, di- and tri-peptide derivatives, peptidyl amino diols and peptidyl beta-aminoacyl aminodiol carbamates; also, and small molecule renin inhibitors including diol sulfonamides and, N-morpholino derivatives, N-heterocyclic alcohols and pyrolimidazolones; also, pepstatin derivatives and fluoro- and chloro-derivatives of statone-containing peptides, enalkrein, remikiren, A 65317, terlakiren, ES 1005, ES 8891, SQ 34017, SPP600, SPP630 and SPP635), endothelin receptor antagonists, phosphodiesterase-5 inhibitors (e.g., sildenafil, tadalfil and vardenafil), vasodilators, calcium channel blockers (e.g., amlodipine, nifedipine, veraparmil, diltiazem, gallopamil, niludipine, nimodipins, nicardipine), potassium channel activators (e.g., nicorandil, pinacidil, cromakalim, minoxidil, aprilkalim, loprazolam), diuretics (e.g., hydrochlorothiazide), sympatholitics, beta-adrenergic blocking drugs (e.g., propranolol, atenolol, bisoprolol, carvedilol, metoprolol, or metoprolol tartate), alpha adrenergic blocking drugs (e.g., doxazosin, prazosin or alpha methyldopa) central alpha adrenergic agonists, peripheral vasodilators (e.g., hydralazine); lipid lowering agents e.g., HMG-CoA reductase inhibitors such as simvastatin and lovastatin, and pharmaceutically acceptable salts of dihydroxy open ring acid HMG-CoA reductase inhibitors such as atorvastatin (particularly the calcium salt thereof), rosuvastatin (particularly the calcium salt thereof), pravastatin (particularly the sodium salt therof), fluvastatin (particularly the sodium salt thereof), cerivastatin, and pitavastatin; a cholesterol absorption inhibitor such as ezetimibe (ZETIA®) and ezetimibe in combination with any other lipid lowering agents such as the HMG-CoA reductase inhibitors noted above and particularly with simvastatin (VYTORIN®) or with atorvastatin calcium; niacin in immediate-release or controlled release forms, and/or with an HMG-CoA reductase inhibitor; niacin receptor agonists such as acipimox and acifran, as well as niacin receptor partial agonists; anti-cholesterol agents such as PCSK9 inhibitors (alirocumab, evolocumab), Nexletol™ (bempedoic acid, ACL inhibitor), and Vascepa® (Icosapent ethyl); insulin or insulin analogs (e.g., insulin detemir, insulin glulisine, insulin degludec, insulin glargine, insulin lispro and inhalable formulations of each); leptin and leptin derivatives and agonists; amylin and amylin analogs (e.g., pramlintide); sulfonylurea and non-sulfonylurea insulin secretagogues (e.g., tolbutamide, glyburide, glipizide, glimepiride, mitiglinide, meglitinides, nateglinide and repaglinide); metabolic altering agents including insulin and insulin mimetics mentioned above, dipeptidyl peptidase-IV (DPP-4) inhibitors (e.g., sitagliptin, alogliptin, omarigliptin, linagliptin, vildagliptin); insulin sensitizers, including (i) β-klotho/FGFR1 activating monoclonal antibody (e.g., those disclosed in U.S. Pat. No. 10,093,735, the contents of which are hereby incorporated by reference), pan FGFR1-4/KLB modulators, FGF19 analogue (e.g., Aldafermin) (ii) PPARγ agonists, such as the glitazones (e.g., pioglitazone, AMG 131, mitoglitazone, lobeglitazone, rosiglitazone, and balaglitazone), and other PPAR ligands, including (1) PPARα/γ dual agonists (e.g., ZYH2, ZYH1, GFT505, chiglitazar, muraglitazar, aleglitazar, sodelglitazar, and naveglitazar); (2) PPARα agonists such as fenofibric acid derivatives (e.g., gemfibrozil, clofibrate, ciprofibrate, fenofibrate, bezafibrate), (3) selective PPARγ modulators (SPPARγM's), (e.g., such as those disclosed in WO 02/060388, WO 02/08188, WO 2004/019869, WO 2004/020409, WO 2004/020408, and WO 2004/066963); (4) PPARγ partial agonists, (5) PPAR α/δ dual agonists (e.g., elafibranor); (iii) biguanides, such as metformin and its pharmaceutically acceptable salts, in particular, metformin hydrochloride, and extended-release formulations thereof, such as Glumetza™, Fortamet™, and GlucophageXR™; and (iv) protein tyrosine phosphatase-1B (PTP-1B) inhibitors (e.g., ISIS-113715 and TTP814); α-glucosidase inhibitors (e.g., acarbose, voglibose and miglitol); PPAR modulators such as PPAR pan agonists (e.g., lanifibranor), PPARα agonists (e.g., pemafibrate), PPARγ agonist (e.g., CHS 131), MPC inhibitor (e.g., PXL065), PPAR α/γ agonist (e.g., T3D 959)); glucagon receptor antagonists (e.g., 3-(4-((1R,2S)-1-(4-chlorophenyl)-1-(7-fluoro-5-methyl-1H-indol-3-yl)pentan-2-yl)benzamido)propanoic acid, N-[(4-{(1S)-1-[3-(3,5-dichlorophenyl)-5-(6-methoxynaphthalen-2-yl)-1H-pyrazol-1-yl]ethyl}phenyl)carbonyl]-β-alanine, adomeglivant and KT6-971); incretin mimetics, bile acid sequestering agents (e.g., colestilan, colestimide, colesevalam hydrochloride, colestipol, cholestyramine, and dialkylaminoalkyl derivatives of a cross-linked dextran), acyl CoA: cholesterol acyltransferase inhibitors, (e.g., avasimibe); antiobesity compounds; agents intended for use in inflammatory conditions, such as aspirin, non-steroidal anti-inflammatory drugs or NSAIDs, glucocorticoids, and selective cyclooxygenase-2 or COX-2 inhibitors; glucokinase activators (GKAs) (e.g., AZD6370); inhibitors of 11β-hydroxysteroid dehydrogenase type 1, (e.g., such as those disclosed in U.S. Pat. No. 6,730,690, and LY-2523199); CETP inhibitors; inhibitors of fructose 1,6-bisphosphatase, (e.g., such as those disclosed in U.S. Pat. Nos. 6,054,587; 6,110,903; 6,284,748; 6,399,782; and 6,489,476); inhibitors of acetyl CoA carboxylase-1 or 2 (ACC1 or ACC2); AMP-activated Protein Kinase (AMPK) activators; other agonists of the G-protein-coupled receptors: (i) GPR-109, (ii) GPR-119 (e.g., MBX2982 and PSN821), and (iii) GPR-40 (e.g., TAK875); SSTR3 antagonists (e.g., such as those disclosed in WO 2009/001836); neuromedin U receptor agonists (e.g., such as those disclosed in WO 2009/042053, including, but not limited to, neuromedin S (NMS)); SCD modulators (e.g., Aramchol); GPR-105 antagonists (e.g., such as those disclosed in WO 2009/000087); glucose pathway modulators such as SGLT inhibitors (e.g., ASP1941, SGLT-3, SGLT-2 such as empagliflozin, dapagliflozin, canagliflozin, and ertugliflozin, BI-10773, remogloflozin, TS-071, tofogliflozin, ipragliflozin, and LX-4211); dual SGLT-1/2 inhibitor (e.g., licogliflozin), Glucose-6-P dehydrogenase inhibitor (e.g., fluasterone) LAPS glucagon combo (e.g., HM14320), SGLT-1 inhibitor (e.g., SGL5213)); inhibitors of acyl coenzyme A carboxylase (ACC, 5-(1′-(1-cyclopropyl-4-methoxy-3-methyl-1H-indole-6-carbonyl)-4-oxospiro[chromane-2,4′-piperidin]-6-yl)nicotinic acid); inhibitors of diacylglycerol acyltransferase 1 and 2 (DGAT-1 and DGAT-2); inhibitors of fatty acid synthase; inhibitors of acyl coenzyme A: monoacylglycerol acyltransferase 1 and 2 (MGAT-1 and MGAT-2); agonists of the TGR5 receptor (also known as GPBAR1, BG37, GPCR19, GPR131, and M-BAR); ileal bile acid transporter inhibitors; bile acid modulators; PACAP, PACAP mimetics, and PACAP receptor 3 agonists; IL-1b antibodies, (e.g., XOMA052 and canakinumab); anti-fibrotic and/or anti-inflammatory agents (CCR2/CCR5 dual receptor antagonist (e.g., cenicriviroc); galectin 3 inhibitor (e.g., belapectin, GB-1107, GB-1211), siRNA against HSP 47 (e.g., BMS-986263); NSAID derived from pirfenidone (e.g., hydronidone), A3AR agonist (e.g., namodenoson, FM101); TGFTX4 (e.g., nitazoxanide); 5-lipoxygenase inhibitor (e.g., tipelukast), bifunctional urate inhibitor (e.g., ACQT1127), adiponectin receptor agonist (e.g., ALY688), TNF receptor antagonist (e.g., atrosimab), autotaxin inhibitor (e.g., BLD-0409, TJC 0265, TJC 0316), CCL24 blocking monoclonal antibody (e.g., CM101), IL-11 inhibitor (e.g., ENx 108A), LPA1 receptor antagonist (e.g., EPGN 696), Dual JAK1/2 inhibitor (e.g., EX 76545), GPR antagonist (e.g., GPR91 antagonist), integrin avb1, avb3 and avb6 inhibitor (e.g., IDL 2965), NLRP3 antagonist (e.g., IFM-514), inflammasome inhibitors (e.g., JT194, JT349), Cell membrane permeability inhibitor (e.g., larazotide), CCR5 antagonist (e.g., leronlimab), TNF inhibitor (e.g., LIVNate), integrin avβ6 inhibitor (e.g., MORF beta6), NLRP inflammasome antagonists, siRNA (e.g., OLX 701), dual TFGβ/Hedgehog inhibitor (e.g., Oxy 200), GPR40 agonist/GPR84 antagonist (e.g., PBI-4547), neutrophil elastase inhibitor (e.g., PHP-303), integrin inhibitor (e.g., PLN-1474), TGFβ1 modulator (e.g., PRM-151), CCK receptor antagonist (e.g., proglumide), LOXL2 inhibitor (e.g., PXS-5338K, PXS-5382A), MPYS protein inhibitor (e.g., cGAS/STING antagonists), kinase inhibiting RNase, membrane protein mAbs, tumor necrosis factor inhibitor, NRF2 activator (e.g., SCO 116), SSAO inhibitor (e.g., TERN 201), TRAIL2 agonist (e.g., TLY012), IL-6 receptor antagonist (e.g., TZLS 501), AOC3 inhibitor (e.g., UD-014), SSAO/VAP-1 inhibitor, TREM2); anti-oxidant (e.g., vitamin E); anti-inflammatory agents (e.g., norfloxacin, ciprofloxacin, ceftriaxone); coagulation modifiers (e.g., anti-coagulants, anti-platelet agents, pentoxifylline, vitamin K, DDAVP); GRP120 stimulant/inflammasome modulator/PPARg dual agonist (e.g., KDT501); selective thyroid hormone receptor-β agonist (e.g., resmetirom); apoptosis modulators (JNK-1 inhibitor (e.g., CC-90001), peroxidase inhibitor (e.g., AZM198), ASK-1 inhibitor (e.g., CS-17919, SRT 015); erythropoietin-stimulating agents (erythropoietin receptor agonist (e.g., cibinetide)); immune modulators (TLR4 inhibitor (e.g., GBK-233), immunomodulatory polyclonal antibody (e.g., IMM-124E), TLR4 antagonist (e.g., JKB-122), CD3 monoclonal antibody (e.g., foralumab), TLR4 antagonist (e.g., JKB 133), TLR4 inhibitor (e.g., mosedipimod), macrophage inhibitor via CD206 targeting (e.g., MT2002), TLR2/4 antagonist (e.g., VB-201, VB-703), immunomodulatory polyclonal antibody (e.g., IMM-124E)); prandial insulin (e.g., ORMD 0801)); lipid modulators (AMPK activator/glutathione transferase (e.g., oltipraz), THR-beta agonist (e.g., resmetirom, VK2809, MGL-3745, ALG-009, ASC41, CNPT-101101, TERN 501), BAT inhibitor (e.g., elobixibat, CJ 14199), omega-6-fatty acid (e.g., epeleuton), FASN inhibitor (e.g., TVB2640, FT 4101, FT 8225), ANGPTL3 inhibitor (e.g., vupanorsen), PNPLA3 inhibitor (e.g., AZD2693), RAS domain kinase inhibitor (e.g., BioE1115), NTCP inhibitor (e.g., bulevirtide), P2Y13 receptor agonist (e.g., CER-209), omega-3 fatty acid, HSD17 β13 inhibitor; metabolism modulators (FXR agonist (e.g., obeticholic acid, IOT022), recombinant variant of FGF19 (e.g., aldafermin), bi-specific FGFR1/KLB antibody (e.g., BFKB8488A), mTOT modulator (e.g., MSDC-0602K), pegylated analog of FGF21 (e.g., pegbelfermin, BMS-986171), non-bile FXR agonist (e.g., cilofexor, EDP-305, EYP 001, tropifexor, MET409, AGN-242256, AGN-242266, EDP 297, HPG 1860, MET642, RDX023, TERN 101), ACC inhibitor (e.g., firsocostat, PF-05221304), ketohexokinase inhibitor (e.g., PF-06835919), AMPK activator (e.g., PXL770, MSTM 101, 0304), bile acid modulator (e.g., Albiero), FGF21 analog (e.g., BIO89-100), MOTSc analog (e.g., CB4211), cyclophilin inhibitor (e.g., CRV 431, NV556), FGF19 (e.g., DEL 30), mitochondrial uncoupler (e.g., GEN 3026), FXR/GPCR dual agonist (e.g., INT-767), cysteamine derivative (e.g., KB-GE-001), dual amylin and calcitonin receptor agonist (e.g., KBP-089), transient FXR agonist (e.g., M 1217), anti-beta-klotho (KLB)-FGFRlc receptor complex mAb (e.g., those disclosed in U.S. Pat. No. 10,093,735), GDF15 analog (e.g., NGM395), LXR modulator (e.g., PX 329, PX 655, PX 788), LXR inverse agonist (e.g., PX016), deuterated obeticholic acid (e.g., ZG 5216)); RAAS mIMModulators (mineralocorticoid receptor antagonist (e.g., apararenone, eplerenone, or spironolactone), angiotensin receptor blocker (e.g., losartan potassium)); neurotransmitter modulators (cannabinoid receptor modulator, CB1 receptor antagonist (e.g., CRB-4001, IM-102, nimacimab), TPH1 inhibitor (e.g., CU 02), GPR120 agonist (e.g., KBR2001), combination of cannabinoid and botanical anti-inflammatory compound (e.g., SCN 002)); PDE modulator (PDE4 inhibitor (e.g., ART 648)); CYP2E1 inhibitor (e.g., SNP-610); cell therapies (e.g., liver-derived mesenchymal stem cells such as HepaStem®)and bromocriptine mesylate and rapid-release formulations thereof, or with other drugs beneficial for the prevention or the treatment of the above-mentioned diseases including nitroprusside and diazoxide the free-acid, free-base, and pharmaceutically acceptable salt forms of the above active agents where chemically possible.

Preferred examples of additional active agents include GIP modulators, AMYR agonists, APLNR agonists and PDE10 inhibitors.

### Dosages of the Compounds of the Present Disclosure

The dosage regimen utilizing a compound of the present disclosure is selected in accordance with a variety of factors including type, species, age, weight, sex and medical condition of the patient; the severity of the condition to be treated; the potency of the compound chosen to be administered; the route of administration; and the renal and hepatic function of the patient. A consideration of these factors is well within the purview of the ordinarily skilled clinician for the purpose of determining the therapeutically effective or prophylactically effective dosage amount needed to prevent, counter, or arrest the progress of the condition. It is understood that a specific daily dosage amount can simultaneously be both a therapeutically effective amount, e.g., for treatment of an immunological condition, and a prophylactically effective amount, e.g., for prevention of an immunological condition.

While individual needs vary, determination of optimal ranges of effective amounts of the compound of the present disclosure is within the skill of one of ordinary skill in the art. For administration to a human in the curative or prophylactic treatment of the conditions and disorders identified herein, for example, typical dosages of the compounds of the present disclosure can be about 0.05 mg/kg/day to about 1000 mg/kg/day. Such doses may be administered in a single dose or may be divided into multiple doses.

For administration to a human in the curative or prophylactic treatment of the conditions and disorders identified herein, for example, typical dosages of the compounds of the present disclosure can be preferably 0.025-7.5 mg/kg/day, more preferably 0.1-2.5 mg/kg/day, and most preferably 0.1-0.5 mg/kg/day (unless specified otherwise, amounts of active ingredients are on free base basis). For example, an 80 kg patient would receive between about 0.8 mg/day and 2.4 g/day, preferably 2-600 mg/day, more preferably 8-200 mg/day, and most preferably 8-40 mg/kg/day. A suitably prepared medicament for once a day administration would thus contain between 0.8 mg and 2.4 g, preferably between 2 mg and 600 mg, more preferably between 8 mg and 200 mg, and most preferably between 8 mg and 100 mg, e.g., 8 mg, 10 mg, 20 mg, 30 mg, 40 mg, 50 mg, 60 mg, 70 mg, 80 mg, 90 mg and 100 mg. Advantageously, the compounds may be administered in divided doses of two, three, or four times daily. For administration twice a day, a suitably prepared medicament would contain between 0.4 mg and 4 g, preferably between 1 mg and 300 mg, more preferably between 4 mg and 100 mg, and most preferably between 4 mg and 50 mg, e.g., 4 mg, 5 mg, 10 mg 15 mg, 20 mg, 25 mg, 30 mg, 35 mg, 45 mg, and 50 mg.

### Pharmaceutical Compositions

The compounds of the disclosure and their pharmaceutically acceptable salts can be administered to animals, preferably to mammals, and particularly to humans, as pharmaceuticals by themselves, in mixtures with one another or in the form of pharmaceutical compositions. The term “subject” or “patient” includes animals, preferably mammals and especially humans, who use the instant active agents for the prevention or treatment of a medical condition.

Administering of the drug to the subject includes both self-administration and administration to the patient by another person. The subject may be in need of or desire treatment for an existing disease or medical condition or may be in need of or desire prophylactic treatment to prevent or reduce the risk of occurrence of the disease or medical condition. As used herein, a subject “in need” of treatment of an existing condition or of prophylactic treatment encompasses both a determination of need by a medical professional as well as the desire of a patient for such treatment.

It is understood that all possible tautomeric forms of the compounds of Formula I are contemplated as being within the scope of the instant invention, as well as mixtures thereof. It is further understood that while only one said tautomeric form of each example compound and embodiment of the invention may be depicted in the specification and appended claims, such depiction includes reference to all tautomeric forms of said compounds, which are included within the scope of the invention.

### EXAMPLES

The following examples are meant to be illustrative and should not be construed as further limiting. The contents of the figures and all references, patents, and published patent applications cited throughout this application are expressly incorporated herein by reference.

### Abbreviations

Abbreviations and acronyms employed herein include the following:

| | |
|---|---|
| AcOH = acetic acid | M = molar |
| Ac = acetyl group | Me = methyl |
| ACN = acetonitrile | |
| aq or aq. = aqueous | MeCN or CH₃CN = acetonitrile |
| Ar = Argon | MeI = iodomethane |
| B2Pin2 = | PPh3 = triphenylphosphane |
| Boc = tert-butyloxycarbonyl | MeOH = methanol |
| BrettPhos = 2-(Dicyclohexylphosphino)3,6- | mg = milligrams |
| dimethoxy-2′,4′,6′-triisopropyl-1,1′- | |
| biphenyl | |
| BrettPhos Pd G3 = [(2-Di- | min or min. = minute |
| cyclohexylphosphino-3,6-dimethoxy- | |
| 2′,4′,6′- triisopropyl-1,1′-biphenyl)-2-(2′- | |
| amino-1,1′-biphenyl)]palladium(II) | |
| methanesulfonate methanesulfonate | |
| n-BuLi = n-butyllithium | MS = mass spectrometry |
| ° C. = degree Celsius | Ms = methanesulfonyl group |
| CDI = N,N′-carbonyldiimidazole | MTBE = methyl tert-butyl ether |
| CSH = charged surface hybrid | mL = milliliters |
| DAST = diethylaminosulfur trifluoride | mmol = millimoles |
| DBU = 1,8-Diazabicyclo[5.4.0]undec- | MsCl = methanesulfonyl chloride |
| 7-ene | |
| DCE = 1,2-dichloroethane | N = normal |
| DCM = dichloromethane | nM = nanomolar |
| DEA = diethylamine | NMP = N-methylpyrrolidone |
| DIPEA or DIEA = N,N- | NMR = nuclear magnetic resonance |
| diisopropylethylamine | |
| dppf = 1,1′- | |
| bis(diphenylphosphino)ferrocene | |
| DMA = dimethylacetamide | PE or Pet. ether = petroleum ether |
| DPPA = diphenylphosphoryl azide | TCFH = N,N,N′,N′- |
| | Tetramethylchloroformamidinium |
| | hexafluorophosphate |
| DMF = N,N-dimethylformamide | |
| DMSO = dimethyl sulfoxide | Pd/C = palladium on carbon |
| Dtbpf = 1,1′-Bis(di-tert- | Prep. or prep = preparative |
| butylphosphino)ferrocene | |
| Equiv = equivalent | psi = pounds per square inch |
| ESI: electrospray ionization | Rt, rt, or RT = room temperature |
| Et₃N = triethyl amine | SFC = Supercritical Fluid Chromatography |
| EtOAc = ethyl acetate | sat aq or sat. aq. = saturated aqueous |
| EtOH = ethanol | sol = solution |
| Et₂O = diethyl ether | TBAF = tetra-N-butylammonium fluoride |
| Ex. = Example | TBAI = tetra-n-butylammonium iodide |
| g = grams | TLC = thin layer chromatography |
| H or h or hr(s) = hour(s) | TEA = triethylamine |
| Hex = hexane | TFA = trifluoroacetic acid |
| HPLC = high performance liquid | t-AmOH = tert-Amyl alcohol |
| chromatography | |
| HATU = 1-[bis(dimethylamino)- | t-BuOH = tert-butanol |
| methylene]-1H-1,2,3-triazolo[4,5- | |
| b]pyridinium-3-oxidehexa- | |
| fluorophosphate | |
| IPA = isopropyl alcohol | THF = tetrahydrofuran |
| (Ir[dF(CF₃)ppy]₂(dtbpy))PF₆ = [4,4′- | TFAA = Trifluoroacetic anhydride |
| Bis(1,1-dimethylethyl)-2,2′-bipyridine- | |
| N1,N1′]bis[3,5-difluoro-2-[5- | |
| (trifluoromethyl)-2-pyridinyl- | |
| N]phenyl-C]Iridium(III) | |
| hexafluorophosphate | |
| | Ts = tosyl group, toluenesulfonyl |
| KHMDS = Potassium | UV = ultraviolet |
| bis(trimethylsilyl)amide | |
| L = liter | v/v = volume per volume |
| LC/MS = liquid chromatography -mass | Wt = weight |
| spectrometry | |
| LiHMDS = lithium | ~ = approximately |
| bis(trimethylsilyl)amide | |
| LDA = lithium diisopropylamide | XPhos Pd G2 = Chloro(2- |
| | dicyclohexylphosphino-2′,4′,6′-triisopropyl- |
| | 1,1′-biphenyl)[2-(2′-amino-1,1′- |
| | biphenyl)]palladium(II) |
| LCMS = Liquid Chromatography | 4CzIPN = 1,2,3,5-Tetrakis(carbazol-9-yl)- |
| Mass spectroscopy | 4,6-dicyanobenzene |

### Intermediates

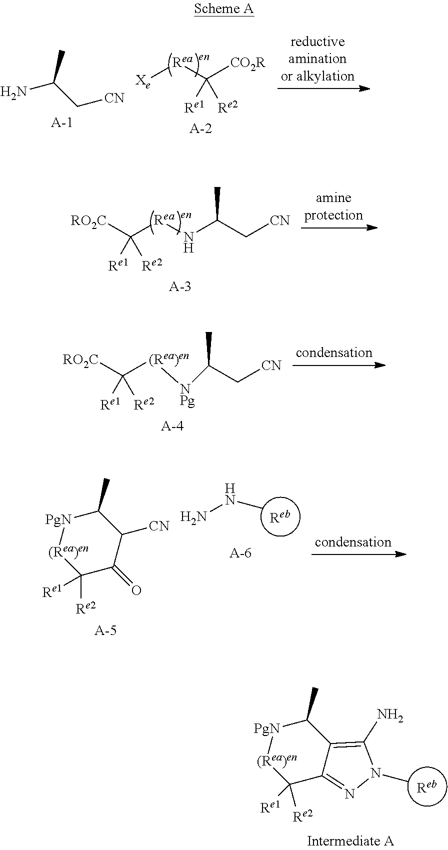

Xₑ is CHO, halide, OTs, or OMs; R^{ea} is alkyl group substituted with 1 or 2 substituents to include H, alkyl, cycloalkyls, OR, F, NRR, or can be combined in a cycloalkyl or saturated heterocycle; n is 1 or 2; R^{e2} is aryl, heteroaryl, linear alkyl, cycloalkyl or saturated heterocycle.

Intermediate A is prepared according to Scheme A via reductive amination or alkylation of intermediate A-2 with amine A-1 to yield intermediate A-3. Protection of A-3 provides intermediate A-4, which is subsequently transformed by a condensation reaction to ketone A-5. Treatment of A-5 with known or synthesized hydrazines (A-6) provides intermediate A.

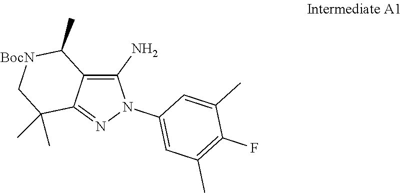 [SMILES: Cc1cc(-n2nc3c(c2N)[C@H](C)N(C(=O)OC(C)(C)C)CC3(C)C)cc(C)c1F]

### tert-butyl (S)-3-amino-2-(4-fluoro-3,5-dimethylphenyl)-4,7,7-trimethyl-2,4,6,7-tetrahydro-5H-pyrazolo[4,3-c]pyridine-5-carboxylate (Scheme A)

### Step 1: methyl (S)-3-((1-cyanopropan-2-yl)amino)-2,2-dimethylpropanoate

To a solution of (S)-3-aminobutanenitrile hydrochloride (71.0 g, 589 mmol) and methyl 2,2-dimethyl-3-oxopropanoate (76.6 g, 589 mmol) in 1,2-dichlorethane (2.10 L) stirred at 25° C. was added NaOAc (48.3 g, 589 mmol) and NaBH(OAc)₃ (187 g, 883 mmol). The reaction mixture was stirred for 12 h at 25° C. Upon reaction completion, the mixture was quenched with NaHCO₃ (saturated aq., 3 L) and extracted with CH₂Cl₂ (3×1 L). The combined organic layers were dried over anhydrous Na₂SO₄, filtered, and then concentrated under reduced pressure. The crude residue was purified by silica gel chromatography (petroleum ether/EtOAc: 1/0 to 0/1) to provide the title compound. ¹H NMR: (400 MHz, DMSO-d6) δ 3.58 (s, 3H), 2.78-2.84 (m, 1H), 2.54-2.63 (m, 2H), 2.50-2.52 (m, 2H), 1.68-1.69 (m, 1H), 1.09 (d, J=0.8 Hz, 6H), 1.06 (t, J=3.2 Hz, 4H). TLC Rf 0.3 (petroleum ether/EtOAc: 3/1, KMnO₄).

### Step 2: methyl (S)-3-((tert-butoxycarbonyl)(1-cyanopropan-2-yl)amino)-2,2-dimethylpropanoate

To a solution of methyl (S)-3-((1-cyanopropan-2-yl)amino)-2,2-dimethylpropanoate (109 g, 548 mmol) in dioxane/H₂O (1/2, 750 mL) at 25° C. was added K₂CO₃ (4 M, 270 mL, 1.97 eq) and Boc₂O (198 g, 908 mol). The reaction mixture was stirred for 12 h at 25° C. The reaction mixture was poured into water (1 L) and extracted with EtOAc (2×300 mL). The combined organic layers were washed with brine (500 mL), dried over anhydrous Na₂SO₄, concentrated under reduced pressure. The crude residue was purified by silica gel chromatography (petroleum ether/EtOAc: 1/0 to 0/1) to provide the title compound. TLC Rf 0.5 (petroleum ether/EtOAc: 3/1, I₂).

### Step 3: tert-butyl (2S)-3-cyano-2,5,5-trimethyl-4-oxopiperidine-1-carboxylate

To a solution of methyl (S)-3-((tert-butoxycarbonyl)(1-cyanopropan-2-yl)amino)-2,2-dimethylpropanoate (72.0 g, 241 mmol) in THF (2.10 L) at 25° C. was added t-BuOK (27.1 g, 241 mmol). The reaction mixture was stirred for 12 h at 25° C. Upon reaction completion, the mixture was quenched with HCl (0.5M aq., 483 mL), diluted with water (2 L), and then extracted with EtOAc (2×1 L). The combined organic layers were washed with brine (1 L), dried over anhydrous Na₂SO₄, filtered and then concentrated under reduced pressure. The crude residue was purified by silica gel chromatography (petroleum ether/EtOAc: 1/0 to 0/1) to provide the title compound. TLC Rf 0.4 (petroleum ether/EtOAc: 3/1, I₂).

### Step 4: tert-butyl (S)-3-amino-2-(4-fluoro-3,5-dimethylphenyl)-4,7,7-trimethyl-2,4,6,7-tetrahydro-5H-pyrazolo[4,3-c]pyridine-5-carboxylate

To a solution of tert-butyl (2S)-3-cyano-2,5,5-trimethyl-4-oxopiperidine-1-carboxylate (53.7 g, 202 mmol) in EtOH (430 mL) at 25° C. was added pyridine (39.9 g, 504 mmol) and (4-fluoro-3,5-dimethylphenyl)hydrazine (46.0 g, 242 mmol). The reaction mixture was stirred for 12 h at 90° C. Upon reaction completion, the mixture was concentrated under reduced pressure and then redissolved in a mixture of MTBE (450 mL) and NaOH (10% aq. 450 mL). The layers were separated, and the aqueous layer was extracted with MTBE (2×150 mL). The combined organic layers were washed with CuSO₄ (aq. 350 mL) and brine (350 mL), dried over anhydrous Na₂SO₄, filtered, and then concentrated under reduced pressure. The crude residue was purified by silica gel chromatography (petroleum ether/EtOAc: 1/0 to 0/1) and then by reverse phase chromatography (neutral conditions) to provide the title compound. ¹H NMR: (400 MHz, DMSO-d6) δ 7.20 (d, J=6.4 Hz, 2H), 5.01-5.16 (m, 3H), 3.67-3.82 (m, 1H), 2.79-2.94 (m, 1H), 2.25 (d, J=1.6 Hz, 6H), 1.42-1.44 (m, 9H), 1.20-1.21 (m, 6H), 1.06-1.09 (m, 3H). MS: 403.3.

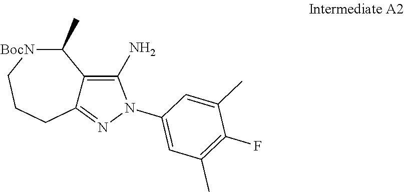 [SMILES: Cc1cc(-n2nc3c(c2N)[C@H](C)N(C(=O)OC(C)(C)C)CCC3)cc(C)c1F]

### tert-butyl (S)-3-amino-2-(4-fluoro-3,5-dimethylphenyl)-4-methyl-2,6,7,8-tetrahydropyrazolo[4,3-c]azepine-5(4H)-carboxylate (Scheme A)

### Step 1: ethyl (S)-4-((1-cyanopropan-2-yl)amino)butanoate

To a solution of 3-aminobutanenitrile (3.9 g, 47 mmol) in DMF (50 mL) was added ethyl 4-bromobutyrate (10 g, 52 mmol), sodium iodide (35 g, 230 mmol) and potassium carbonate (19 g, 140 mmol). The reaction mixture was stirred at 20° C. for 16 h, then filtered and concentrated under reduced pressure to provide the title compound. ¹H NMR (400 MHz, chloroform-d) δ 4.21-4.01 (m, 2H), 2.67-2.60 (m, 1H), 2.48-2.29 (m, 4H), 2.17 (s, 1H), 1.82-1.70 (m, 2H), 1.29-1.17 (m, 6H). MS (ESI) m/z: 199.2 [M+H]+.

### Step 2-4: tert-butyl (S)-3-amino-2-(4-fluoro-3,5-dimethylphenyl)-4-methyl-2,6,7,8-tetrahydropvrazolo[4,3-c]azepine-5(4H)-carboxylate

The procedures described for preparation of Intermediate A1 were followed for ethyl (S)-4-((1-cyanopropan-2-yl)amino)butanoate to provide the title compound. ¹H NMR (CH₃OH-d4, 400 MHz): δ 7.15 (2H, d, J=6.3 Hz), 5.28-5.30 (1H, in), 4.09 (1H, d, J=15.0 Hz), 3.36 (1H, d, J=13.0 Hz), 2.71-2.77 (2H, n), 2.28 (6H, d, J=2.2 Hz), 1.62-1.93 (2H, m), 1.40-1.45 (12H, m). MS (ESI) m/z: 389.2 [M+H]+.

TABLE A

| Intermediate | Structure | Name | MS (M + 1) |
|---|---|---|---|
| A3 | 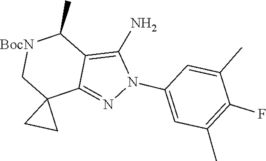 [SMILES: Cc1cc(-n2nc3c(c2N)[C@H](C)N(C(=O)OC(C)(C)C)CC32CC2)cc(C)c1F] | tert-butyl (S)-3′-amino-2′-(4-fluoro- 3,5-dimethylphenyl)-4′-methyl-2′,4′- dihydrospiro[cyclopropane-1,7′- pyrazolo[4,3-c]pyridine]-5′(6′H)- carboxylate | 401.3 |
| A4 | 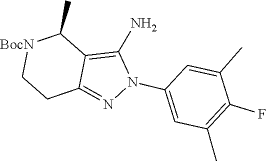 [SMILES: Cc1cc(-n2nc3c(c2N)[C@H](C)N(C(=O)OC(C)(C)C)CC3)cc(C)c1F] | tert-butyl (S)-3-amino-2-(4-fluoro- 3,5-dimethylphenyl)-4-methyl- 2,4,6,7-tetrahydro-5H-pyrazolo[4,3- c]pyridine-5-carboxylate | 375.3 |
| A5 | 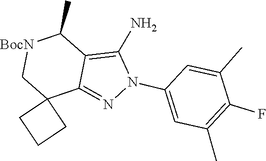 [SMILES: Cc1cc(-n2nc3c(c2N)[C@H](C)N(C(=O)OC(C)(C)C)CC32CCC2)cc(C)c1F] | tert-butyl (S)-3′-amino-2′-(4-fluoro- 3,5-dimethylphenyl)-4′-methyl-2′,4′- dihydrospiro[cyclobutane-1,7′- pyrazolo[4,3-c]pyridine]-5′(6′H)- carboxylate | 415.4 |
| A6 | 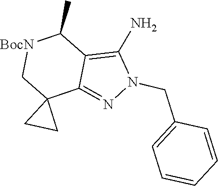 [SMILES: C[C@H]1c2c(nn(Cc3ccccc3)c2N)C2(CC2)CN1C(=O)OC(C)(C)C] | tert-butyl (S)-3′-amino-2′-benzyl-4′- methyl-2′,4′- dihydrospiro[cyclopropane-1,7′- pyrazolo[4,3-c]pyridine]-5′(6′H)- carboxylate | 369.1 |
| A7 | 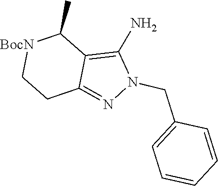 [SMILES: C[C@H]1c2c(nn(Cc3ccccc3)c2N)CCN1C(=O)OC(C)(C)C] | tert-butyl (S)-3-amino-2-benzyl-4- methyl-2,4,6,7-tetrahydro-5H- pyrazolo[4,3-c]pyridine-5- carboxylate | 343.2 |
| A8 | 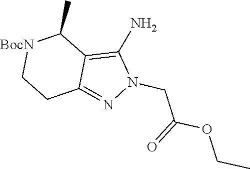 [SMILES: CCOC(=O)Cn1nc2c(c1N)[C@H](C)N(C(=O)OC(C)(C)C)CC2] | tert-butyl (S)-3-amino-2-(2-ethoxy- 2-oxoethyl)-4-methyl-2,4,6,7- tetrahydro-5H-pyrazolo[4,3- c]pyridine-5-carboxylate | 339.1 |
| A9 | 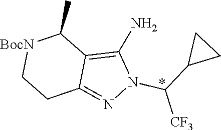 [SMILES: C[C@H]1c2c(nn(C(C3CC3)C(F)(F)F)c2N)CCN1C(=O)OC(C)(C)C] | tert-butyl (S)-3-amino-2-((S)-1- cyclopropyl-2,2,2-trifluoroethyl)-4- methyl-2,4,6,7-tetrahydro-5H- pyrazolo[4,3-c]pyridine-5- carboxylate and tert-butyl (S)-3- amino-2-((R)-1-cyclopropyl-2,2,2- trifluoroethyl)-4-methyl-2,4,6,7- tetrahydro-5H-pyrazolo[4,3- c]pyridine-5-carboxylate | 375.1 |
| A10-1 | 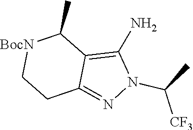 [SMILES: C[C@H](n1nc2c(c1N)[C@H](C)N(C(=O)OC(C)(C)C)CC2)C(F)(F)F] | tert-butyl (S)-3-amino-4-methyl-2- ((S)-1,1,1-trifluoropropan-2-yl)- 2,4,6,7-tetrahydro-5H-pyrazolo[4,3- c]pyridine-5-carboxylate | 349.4 |
| A10-2 | 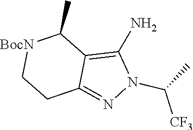 [SMILES: C[C@H]1c2c(nn([C@H](C)C(F)(F)F)c2N)CCN1C(=O)OC(C)(C)C] | tert-butyl (S)-3-amino-4-methyl-2- ((R)-1,1,1-trifluoropropan-2-yl)- 2,4,6,7-tetrahydro-5H-pyrazolo[4,3- c]pyridine-5-carboxylate | 349.2 |
| A11 | 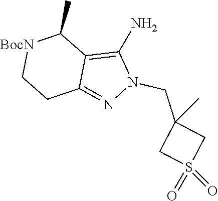 [SMILES: C[C@H]1c2c(nn(CC3(C)CS(=O)(=O)C3)c2N)CCN1C(=O)OC(C)(C)C] | tert-butyl (S)-3-amino-4-methyl-2- ((3-methyl-1,1-dioxidothietan-3- yl)methyl)-2,4,6,7-tetrahydro-5H- pyrazolo[4,3-c]pyridine-5- carboxylate | 385.1 |
| A12 | 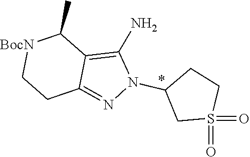 [SMILES: C[C@H]1c2c(nn(C3CCS(=O)(=O)C3)c2N)CCN1C(=O)OC(C)(C)C] | tert-butyl (S)-3-amino-2-((S)-1,1- dioxidotetrahydrothiophen-3-yl)-4- methyl-2,4,6,7-tetrahydro-5H- pyrazolo[4,3-c]pyridine-5- carboxylate and tert-butyl (S)-3- amino-2-((R)-1,1- dioxidotetrahydrothiophen-3-y1)-4- methyl-2,4,6,7-tetrahydro-5H- pyrazolo[4,3-c]pyridine-5-carboxylate | 371.4 |
| A13 | 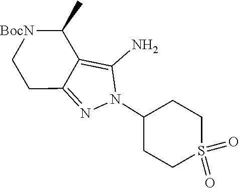 [SMILES: C[C@H]1c2c(nn(C3CCS(=O)(=O)CC3)c2N)CCN1C(=O)OC(C)(C)C] | tert-butyl (S)-3-amino-2-(1,1- dioxidotetrahydro-2H-thiopyran-4- yl)-4-methyl-2,4,6,7-tetrahydro-5H- pyrazolo[4,3-c]pyridine-5- carboxylate | 385.4 |
| A14 | 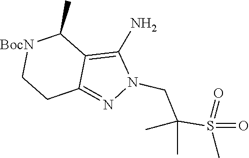 [SMILES: C[C@H]1c2c(nn(CC(C)(C)S(C)(=O)=O)c2N)CCN1C(=O)OC(C)(C)C] | tert-butyl (S)-3-amino-4-methyl-2- (2-methyl-2- (methylsulfonyl)propyl)-2,4,6,7- tetrahydro-5H-pyrazolo[4,3- c]pyridine-5-carboxylate | 387.4 |
| A15 | 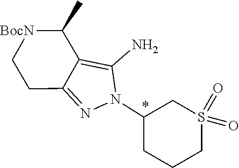 [SMILES: C[C@H]1c2c(nn(C3CCCS(=O)(=O)C3)c2N)CCN1C(=O)OC(C)(C)C] | tert-butyl (S)-3-amino-2-((R)-1,1- dioxidotetrahydro-2H-thiopyran-3- yl)-4-methyl-2,4,6,7-tetrahydro-5H- pyrazolo[4,3-c]pyridine-5- carboxylate and tert-butyl (S)-3- amino-2-((S)-1,1-dioxidotetrahydro- 2H-thiopyran-3-yl)-4-methyl- 2,4,6,7-tetrahydro-5H-pyrazolo[4,3- c]pyridine-5-carboxylate | 385.4 |
| A16 | 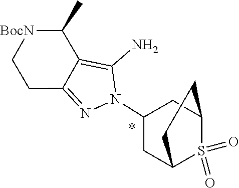 [SMILES: C[C@H]1c2c(nn(C3C[C@H]4CC[C@@H](C3)S4(=O)=O)c2N)CCN1C(=O)OC(C)(C)C] | tert-butyl (S)-3-amino-2-((1R,3r,5S)- 8,8-dioxido-8- thiabicyclo[3.2.1]octan-3-yl)-4- methyl-2,4,6,7-tetrahydro-5H- pyrazolo[4,3-c]pyridine-5- carboxylate and tert-butyl (S)-3- amino-2-((1R,3s,5S)-8,8-dioxido-8- thiabicyclo[3.2.1]octan-3-yl)-4- methyl-2,4,6,7-tetrahydro-5H- pyrazolo[4,3-c]pyridine-5- carboxylate | 411.2 |
| A17 | 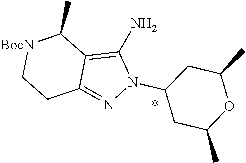 [SMILES: C[C@@H]1CC(n2nc3c(c2N)[C@H](C)N(C(=O)OC(C)(C)C)CC3)C[C@H](C)O1] | tert-butyl (S)-3-amino-2-((2R,4r,6S)- 2,6-dimethyltetrahydro-2H-pyran-4- yl)-4-methyl-2,4,6,7-tetrahydro-5H- pyrazolo[4,3-c]pyridine-5- carboxylate and tert-butyl (S)-3- amino-2-((2R,4s,6S)-2,6- dimethyltetrahydro-2H-pyran-4-yl)- 4-methyl-2,4,6,7-tetrahydro-5H- pyrazolo[4,3-c]pyridine-5- carboxylate | 365.2 |
| A18 | 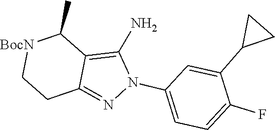 [SMILES: C[C@H]1c2c(nn(-c3ccc(F)c(C4CC4)c3)c2N)CCN1C(=O)OC(C)(C)C] | tert-butyl (S)-3-amino-2-(3- cyclopropyl-4-fluorophenyl)-4- methyl-2,4,6,7-tetrahydro-5H- pyrazolo[4,3-c]pyridine-5- carboxylate | 387.2 |
| A19 | 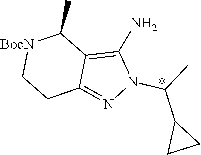 [SMILES: CC(C1CC1)n1nc2c(c1N)[C@H](C)N(C(=O)OC(C)(C)C)CC2] | tert-butyl (S)-3-amino-2-((S)-1- cyclopropylethyl)-4-methyl-2,4,6,7- tetrahydro-5H-pyrazolo[4,3- c]pyridine-5-carboxylate and tert- butyl (S)-3-amino-2-((R)-1- cyclopropylethyl)-4-methyl-2,4,6,7- tetrahydro-5H-pyrazolo[4,3- c]pyridine-5-carboxylate | 321.4 |
| A20 | 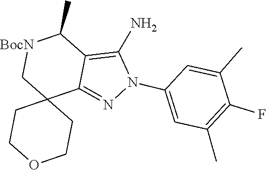 [SMILES: Cc1cc(-n2nc3c(c2N)[C@H](C)N(C(=O)OC(C)(C)C)CC32CCOCC2)cc(C)c1F] | tert-butyl (S)-3′-amino-2′-(4-fluoro- 3,5-dimethylphenyl)-4′-methyl- 2,2′,3,4′,5,6-hexahydrospiro[pyran- 4,7′-pyrazolo[4,3-c]pyridine]- 5′(6′H)-carboxylate | 209.1 (M − Boc + 1) |
| VB-1 | 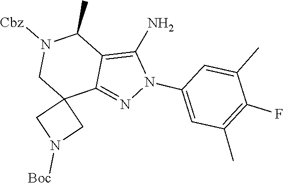 [SMILES: Cc1cc(-n2nc3c(c2N)[C@H](C)N(C(=O)OCc2ccccc2)CC32CN(C(=O)OC(C)(C)C)C2)cc(C)c1F] | 5′-benzyl 1-(tert-butyl) (S)-3′-amino- 2′-(4-fluoro-3,5-dimethylphenyl)-4′- methyl-2′,4′-dihydrospiro[azetidine- 3,7′-pyrazolo[4,3-c]pyridine]- 1,5′(6′H)-dicarboxylate | 494.2 [M − tBu + H]⁺ |
| BW-0 | 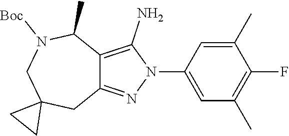 [SMILES: Cc1cc(-n2nc3c(c2N)[C@H](C)N(C(=O)OC(C)(C)C)CC2(CC2)C3)cc(C)c1F] | tert-butyl (S)-3′-amino-2′-(4-fluoro- 3,5-dimethylphenyl)-4′-methyl-2′,8′- dihydro-4′H-spiro[cyclopropane- 1,7′-pyrazolo[4,3-c]azepine]-5′(6′H)- carboxylate | 415.2 |
| MU-5 | 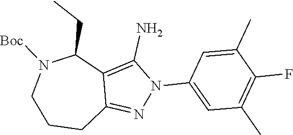 [SMILES: CC[C@H]1c2c(nn(-c3cc(C)c(F)c(C)c3)c2N)CCCN1C(=O)OC(C)(C)C] | tert-butyl (S)-3-amino-4-ethyl-2-(4- fluoro-3,5-dimethylphenyl)-2,6,7,8- tetrahydropyrazolo[4,3-c]azepine- 5(4H)-carboxylate | 403.3 |
| RJ-IB | 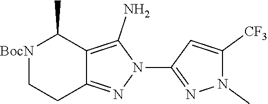 | tert-butyl (S)-3-amino-4-methyl-2- (1-methyl-5-(trifluoromethyl)-1H- pyrazol-3-yl)-2,4,6,7-tetrahydro-5H- pyrazolo[4,3-c]pyridine-5- carboxylate | 401.5 |
| VB-B1 | 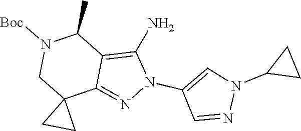 [SMILES: C[C@H]1c2c(nn(-c3cnn(C4CC4)c3)c2N)C2(CC2)CN1C(=O)OC(C)(C)C] | tert-butyl (S)-3′-amino-2′-(1- cyclopropyl-1H-pyrazol-4-yl)-4′- methyl-2′,4′- dihydrospiro[cyclopropane-1,7′- pyrazolo[4,3-c]pyridine]-5′(6′H)- carboxylate | 385.4 |
| VB-B3 | 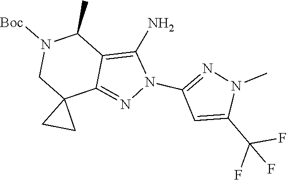 [SMILES: C[C@H]1c2c(nn(-c3cc(C(F)(F)F)n(C)n3)c2N)C2(CC2)CN1C(=O)OC(C)(C)C] | tert-butyl (S)-3′-amino-4′-methyl-2′- (1-methyl-5-(trifluoromethyl)-1H- pyrazol-3-yl)-2′,4′- dihydrospiro[cyclopropane-1,7′- pyrazolo[4,3-c]pyridine]-5′(6′H)- carboxylate | 427.3 |
| VB-B4 | 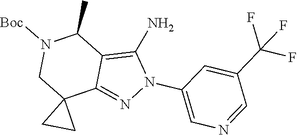 [SMILES: C[C@H]1c2c(nn(-c3cncc(C(F)(F)F)c3)c2N)C2(CC2)CN1C(=O)OC(C)(C)C] | tert-butyl (S)-3′-amino-4′-methyl-2′- (5-(trifluoromethyl)pyridin-3-yl)- 2′,4′-dihydrospiro[cyclopropane-1,7′- pyrazolo[4,3-c]pyridine]-5′(6′H)- carboxylate | 424.5 |

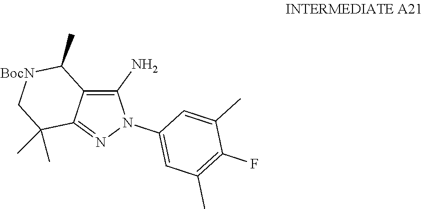 [SMILES: Cc1cc(-n2nc3c(c2N)[C@H](C)N(C(=O)OC(C)(C)C)CC3(C)C)cc(C)c1F]

### tert-butyl (S)-3-amino-7,7-difluoro-2-(4-fluoro-3,5-dimethylphenyl)-4-methyl-2,4,6,7-tetrahydro-5H-pyrazolo[4,3-c]pyridine-5-carboxylate

### Step 1: (S)-3-(benzylamino)butanenitrile

To a solution of (S)-3-aminobutanenitrile hydrochloride (4.0 g, 33 mmol) in DCE (100 mL) was added sodium acetate (5.4 g, 66 mmol) and benzaldehyde (3.5 g, 33 mmol), and stirred at 0° C. for 30 min under an atmosphere of N₂. Then, sodium triacetoxyhydroborate (15 g, 66 mmol) was added and the resulting mixture was stirred at 0° C. for 3 h. The mixture was quenched with sat aq. NH₄Cl (20 mL) and H₂O (50 mL) at 0° C., and extracted with DCM (3×80 mL). The combined organic layers were washed with brine (50 mL), dried over anhydrous Na₂SO₄, filtered, and concentrated under reduced pressure. The crude residue was purified by SiO₂ chromatography (1:1 Pet. ether/EtOAc) to afford the title compound. LCMS m/z[M+H]: 175.2

### Step 2: (S)-3-(((1H-benzo[d][1,2,3]triazol-1-yl)methyl)(benzyl)amino)butanenitrile

To a solution of (S)-3-(benzylamino)butanenitrile (4.0 g, 23 mmol) in ethanol (30 mL) was added (1H-benzo[d][1,2,3]triazol-1-yl)methanol (3.4 g, 23 mmol). The reaction mixture was stirred at 25° C. for 16 h under a N₂ atmosphere. Then, the mixture was concentrated to afford the title compound, which was used in the next step. ¹H NMR (400 MHz, DMSO-d6) δ ppm 8.05 (d, J=8.46 Hz, 1H) 7.75 (d, J=8.46 Hz, 1H) 7.50-7.56 (m, 1H) 7.37-7.43 (m, 3H) 7.35 (t, J=7.33 Hz, 2H) 7.25-7.30 (m, 1H) 5.61-5.71 (m, 1H) 5.50 (d, J=14.31 Hz, 1H) 3.94 (d, J=14.31 Hz, 1H) 3.74-3.83 (m, 1H) 3.41-3.49 (m, 1H) 2.77-2.89 (m, 1H) 2.62-2.72 (m, 1H) 0.83-0.89 (m, 3H).

### Step 3: ethyl (S)-3-(benzyl(1-cyanopropan-2-yl)amino)-2,2-difluoropropanoate

To a solution of (S)-3-(((1H-benzo[d][1,2,3]triazol-1-yl)methyl)(benzyl)amino) butanenitrile (2.0 g, 5.9 mmol) in THF (10 mL) was added (2-ethoxy-1,1-difluoro-2-oxoethyl)zinc(II) bromide (98 mL, 18 mmol) at 25° C. Then, the reaction mixture was stirred for 16 h. The reaction mixture was filtered and the filtrate was concentrated under reduced pressure The crude residue was purified by SiO₂ chromatography (0 to 10% EtOAc/Pet. ether) to afford the title compound. LCMS m/z[M+H]: 311.0

### Step 4: (2S)-1-benzyl-5,5-difluoro-2-methyl-4-oxopiperidine-3-carbonitrile

To a solution of ethyl (S)-3-(benzyl(1-cyanopropan-2-yl)amino)-2,2-difluoropropanoate (200 mg, 0.644 mmol) in THF (2.00 mL) was added lithium diisopropylamide (0.644 mL, 1.29 mmol) at −78° C. under a N₂ atmosphere. The mixture was stirred at −78° C. for 0.5 h, then stirred at 25° C. for 2 h. The mixture was quenched with NH₄Cl (3 mL), extracted with EtOAc (3×10 mL). The combined organic layers were dried over Na₂SO₄, filtered and concentrated under reduced pressure to afford the title compound without further purification. MS (ESI) m/z: 265.0 [M+H]+.

### Step 5: (S)-5-benzyl-7,7-difluoro-2-(4-fluoro-3,5-dimethylphenyl)-4-methyl-4,5,6,7-tetrahydro-2H-pyrazolo[4,3-c]pyridin-3-amine

To a solution of (4-fluoro-3,5-dimethylphenyl)hydrazine (23 mg, 0.15 mmol) in toluene (1.0 mL) was added (2S)-1-benzyl-5,5-difluoro-2-methyl-4-oxopiperidine-3-carbonitrile (40 mg, 0.151 mmol) and pyridine (17 mg, 0.15 mmol). The mixture was stirred at 90° C. for 40 min under an atmosphere of N₂. The reaction was concentrated under reduced pressure and then purified by Prep-TLC (2:1 Pet.ether/EtOAc) and reverse-phase HPLC (36 to 56% MeCN/H₂O+0.1% TFA) to afford the title compound. LCMS m/z[M+H]: 401.1

### Step 6: tert-butyl (S)-3-amino-7,7-difluoro-2-(4-fluoro-3,5-dimethylphenyl)-4-methyl-2,4,6,7-tetrahydro-5H-pyrazolo[4,3-c]pyridine-5-carboxylate

A solution of (S)-5-benzyl-7,7-difluoro-2-(4-fluoro-3,5-dimethylphenyl)-4-methyl-4,5,6,7-tetrahydro-2H-pyrazolo[4,3-c]pyridin-3-amine (30 mg, 0.075 mmol), Boc₂O (0.035 mL, 0.15 mmol) and Pd/C (8.0 mg, 7.5 μmol) in EtOAc (6.0 mL) was stirred at 25° C. under a H₂ atmosphere (via balloon/15 psi) for 16 h. Then, the atmosphere was replaced with an inert atmosphere and the reaction mixture was filtered. The filtrate was concentrated under reduced pressure. The crude residue was purified by Prep-TLC (SiO2, 1/3=EtOAc/petroleum ether) to afford the title compound. LCMS m/z[M+H]: 411.1.

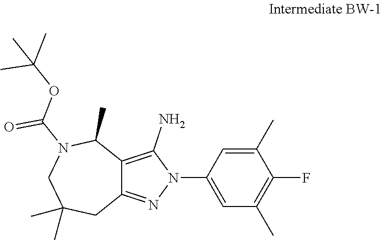 [SMILES: Cc1cc(-n2nc3c(c2N)[C@H](C)N(C(=O)OC(C)(C)C)CC(C)(C)C3)cc(C)c1F]

### Step 1: methyl (S)-2-(1-(((1-cyanopropan-2-yl)amino)methyl)cyclopropyl)acetate

To a 200 mL round-bottomed flask was added (S)-3-aminobutanenitrile hydrochloride (5.300 g, 43.95 mL, 1 molar, 1 equiv, 43.95 mmol), potassium carbonate (18.2 g, 3 equiv, 132 mmol), sodium iodide (33 g, 5 equiv, 220 mmol), and DMF (44 mL) under nitrogen atmosphere. methyl 2-(1-(bromomethyl)cyclopropyl)acetate (9.1 g, 1 equiv, 44 mmol) was then added dropwise, and the resulting heterogeneous mixture stirred rapidly for 18 h. Then, the mixture was diluted with EtOAc (50 mL), filtered over Celite® (a product made from diatomaceous earth) and concentrated. The crude material was used directly in the next step. MS (ESI) m/z: 211.0 [M+H]+

### Step 2: methyl (S)-2-(1-(((1-cyanopropan-2-yl)amino)methyl)cyclopropyl)acetate

To a mixture of methyl (S)-2-(1-(((1-cyanopropan-2-yl)amino)methyl) cyclopropyl)acetate (9.25 g, 1 equiv, 44.0 mmol) and potassium carbonate (12.2 g, 2 equiv, 88.0 mmol) was added 1,4-dioxane (40 mL), water (80 mL), and then Boc₂O (10.1 mL, 1 equiv, 44.0 mmol). The mixture was stirred for 18 h before sat. aq. NaHCO₃ (100 mL) was added, and the resulting biphasic mixture as extracted with EtOAc (3×100 mL), washed with 1M HCl (100 mL), then NaHCO₃ (100 mL). The organic layer was dried over Na₂SO₄ and concentrated. Purified by silica gel flash column chromatography (220 g column, 0 to 80% EtOAc/hexanes, 150 mL/min). Isolated methyl (S)-2-(1-(((tert-butoxycarbonyl)(1-cyanopropan-2-yl)amino)methyl)cyclopropyl)acetate as an oil. MS (ESI) m/z: 233.2 [M+Na]+

### Step 3: tert-butyl (S)-3′-amino-2′-(4-fluoro-3,5-dimethylphenyl)-4′-methyl-2′,8′-dihydro-4′H-spiro[cyclopropane-1,7′-pyrazolo[4,3-c]azepine]-5′(6′H)-carboxylate

To a 250 mL round-bottomed flask was added methyl (S)-2-(1-(((tert-butoxycarbonyl)(1-cyanopropan-2-yl)amino)methyl)cyclopropyl)acetate (1.20 g, 77.3 mL, 1 equiv, 3.866 mmol) and THF (65 mL, 0.05 M) under N₂. Then cooled to −78° C. and KHMDS (1.620 g, 8.119 mL, 1 molar, 2.1 equiv, 8.119 mmol) added dropwise to form a solution. After stirring for 30 min at this temperature, AcOH (1.161 g, 1.107 mL, 5 equiv, 19.33 mmol) added to quench. Then aq. NH₄Cl (50 mL) and EtOAc (50 mL) were added, and the mixture warmed to ambient temperature. The biphasic mixture was extracted with EtOAc (3×40 mL), dried over Na₂SO₄, and concentrated to a colorless oil.

Then to the crude mixture was added (4-fluoro-3,5-dimethylphenyl)hydrazine (655.7 mg, 14.18 mL, 0.3 molar, 1.1 equiv, 4.253 mmol) along with AcOH (1.161 g, 1.107 mL, 5 equiv, 19.33 mmol) and EtOH (12 mL). The vial was capped and heated to 90° C. for 2 h. Then the mixture was concentrated under reduced pressure and directly purified by flash column chromatography (40 g ISCO, 0 to 40% EtOAc/hexanes, 30 mL/min) to give tert-butyl (S)-3′-amino-2′-(4-fluoro-3,5-dimethylphenyl)-4′-methyl-2′,8′-dihydro-4′H-spiro[cyclopropane-1,7′-pyrazolo[4,3-c]azepine]-5′(6′H)-carboxylate. MS (ESI) m/z: 417.6 [M+H]⁺

### Step 4: tert-butyl (S)-3-amino-2-(4-fluoro-3,5-dimethylphenyl)-4,7,7-trimethyl-2,6,7,8-tetrahydropyrazolo[4,3-c]azepine-5(4H)-carboxylate

To a solution of tert-butyl (S)-3′-amino-2′-(4-fluoro-3,5-dimethylphenyl)-4′-methyl-2′,8′-dihydro-4′H-spiro[cyclopropane-1,7′-pyrazolo[4,3-c]azepine]-5′(6′H)-carboxylate in AcOH (1.5 mL) was added platinum(IV) oxide hydrate (30 mg, 0.1 mmol, 0.5 equiv). The mixture was then sparged with H₂ for 10 min and stirred for 7 days under a balloon of H₂. The mixture was then purged with N₂, filtered over Celite®, and concentrated under reduced pressure. The crude residue was purified by flash column chromatography (0 to 70% EtOAc/hexanes) to give tert-butyl (S)-3-amino-2-(4-fluoro-3,5-dimethylphenyl)-4,7,7-trimethyl-2,6,7,8-tetrahydropyrazolo[4,3-c]azepine-5(4H)-carboxylate ¹H NMR (500 MHz, CDCl₃) δ 7.16 (d, J=6.0 Hz, 2H), 6.40 (s, 4H), 5.45 (d, J=6.9 Hz, 1H), 3.83 (d, J=15.0 Hz, 1H), 3.23 (d, J=15.1 Hz, 1H), 2.78-2.66 (m, 2H), 2.28 (d, J=1.6 Hz, 6H), 1.44 (d, J=6.5 Hz, 12H), 1.07 (s, 3H), 0.88 (s, 3H). MS (ESI) m/z: 419.6 [M+H]⁺

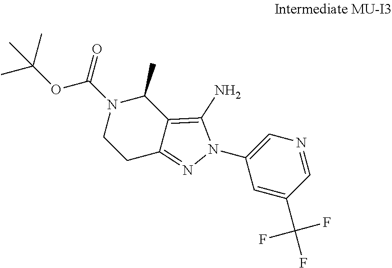 [SMILES: C[C@H]1c2c(nn(-c3cncc(C(F)(F)F)c3)c2N)CCN1C(=O)OC(C)(C)C]

### tert-butyl (S)-3-amino-4-methyl-2-(5-(trifluoromethyl)pyridin-3-yl)-2,4,6,7-tetrahydro-5H-pyrazolo[4,3-c]pyridine-5-carboxylate

### Step 1: 3-hydrazineyl-5-(trifluoromethyl)pyridine

To a solution of 5-Trifluoromethyl-pyridin-3-yl amine (900 mg, 5.55 mmol) in 6N HCl (aq) (9 mL) was added a solution of sodium nitrite (383 mg, 5.55 mmol) in water (5 mL) dropwise over 3 minutes at 0° C. After stirring for 1 hour at 0° C., a solution of dichlorostannane (2.63 g, 682 μL, 2.5 Eq, 13.9 mmol) in 6N HCl (aq) (9 mL) was added dropwise over 5 minutes. After 30 minutes at 0° C., this mixture was stirred at room temperature for 18 hours. The mixture was cooled to 0° C. and 6M aqueous sodium hydroxide (25 ml) was added dropwise. The mixture was extracted with EtOAc (3×25 ml) and the combined organic phase was washed with brine, dried over Na₂SO₄, filtered and concentrated under reduced pressure. MS (ESI) m/z: 178.1 [M+H]+.

### Step 2: tert-butyl (S)-3-amino-4-methyl-2-(5-(trifluoromethyl)pyridin-3-yl)-2,4,6,7-tetrahydro-5H-pyrazolo[4,3-c]pyridine-5-carboxylate

(2S)-tert-Butyl 3-cyano-2-methyl-4-oxopiperidine-1-carboxylate (899 mg, 3.77 mmol), 3-hydrazineyl-5-(trifluoromethyl)pyridine (668 mg, 1 Eq, 3.77 mmol), pyridine hydrochloride (43.6 mg, 377 mol) and dry toluene (10 mL) was heated at 90° C. for 1 h. After cooling to room temperature, the mixture was poured into water (10 mL) and the pH adjusted to 9 with 1M NaOH (aq). The organic phase was extracted with ethyl acetate (10 mL×3), and the combined organic layers were dried over sodium sulfate and concentrated. The crude product was purified over silica by flash column chromatography (40 g silica, 0 to 30% ethyl acetate in heptane). MS (ESI) m/z: 398.1 [M+H]+.

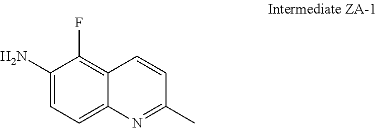

### 5-fluoro-2-methylquinolin-6-amine

To a solution of 2-methylquinolin-6-amine (1.0 g, 6.3 mmol) in MeCN (40 mL) was added 1-chloromethyl-4-fluoro-1,4-diazoniabicyclo[2.2.2]octane bis(tetrafluoroborate)(2.24 g, 6.32 mmol), and the resulting mixture was stirred at 20° C. for 2 hours. The reaction mixture was poured into water (50 mL), and extracted with EtOAc (3×50 mL), washed with brine (2×200 mL), dried over Na₂SO₄, filtered, and concentrated under reduced pressure to afford crude product. The crude material was purified by Prep-HPLC (YMC-Actus Triart C18 MeCN/H₂O (0.1% TFA) 3-100%) to provide the title compound. MS (ESI) m/z: 177.1 [M+H]+.

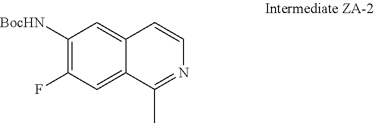 [SMILES: Cc1nccc2cc(NC(=O)OC(C)(C)C)c(F)cc12]

### tert-butyl (7-fluoro-1-methylisoquinolin-6-yl)carbamate

Tris((1E,4E)-1,5-diphenylpenta-1,4-dien-3-one) dipalladium (0.12 g, 0.14 mmol) and dicyclohexyl({3,6-dimethoxy-2-[2,4,6-tris(propan-2-yl)phenyl]phenyl})phosphane (0.15 g, 0.27 mmol) were added to an argon flushed mixture of 6-bromo-7-fluoro-1-methylisoquinoline (0.65 g, 2.7 mmol). To these solids were added tert-butyl carbamate (0.48 g, 4.1 mmol) and cesium carbonate (1.8 g, 5.4 mmol, in 0.43 mL H₂O) followed by dioxane (14 mL). The suspension was stirred for 16 hours at 90° C., filtered, and concentrated under reduced pressure. The crude mixture was purified by silica gel column chromatography (0-100% EtOAc in heptane) to afford the title compound. MS (ESI) m/z: 277.1 [M+H]+.

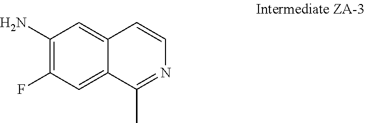

### 7-fluoro-1-methylisoquinolin-6-amine

Hydrogen chloride (4.0 M in 1,4-dioxane) (18 mL, 72 mmol) was added to a solution of tert-butyl (7-fluoro-1-methylisoquinolin-6-yl)carbamate (610 mg, 2.21 mmol) in DCM (18 mL). The mixture was stirred for 16 hours at room temperature. The mixture was then concentrated under reduced pressure to afford the title compound. MS (ESI) m/z: 177.2 [M+H]+.

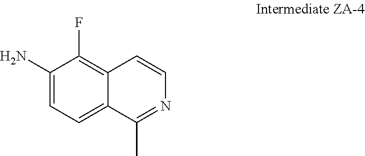

### 5-fluoro-1-methylisoquinolin-6-amine

This procedure was performed in a similar fashion as described for Intermediate ZA-1 to provide the title compound. MS (ESI) m/z: 177.1 [M+H]+.

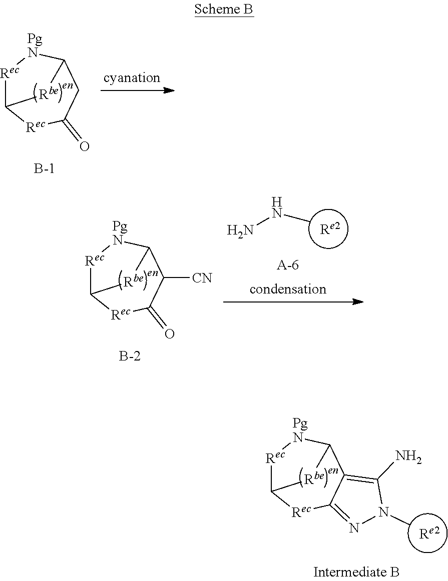

R^{eb} is CH₂ or O; en is 1, 2 or 3; R^{ec} (independently)=CH₂, CHR^{ec1}, or CR^{ec1}R^{ec2}, wherein R^{ec1} and R^{ec2} can form a carbocycle or heterocycle; R2=aryl, heteroaryl, alkyl, cycloalkyl, or heterocycle.

Intermediate B is prepared according to Scheme B via cyanation of intermediate B-1 with cyanide reagents such as p-toluenesulfonyl cyanide to yield intermediate B-2. Subsequently, treatment of B-2 with known or synthesized hydrazines (A-6) provides intermediate B.

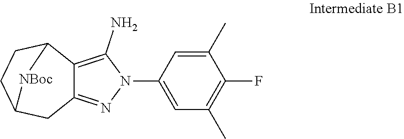 [SMILES: Cc1cc(-n2nc3c(c2N)C2CCC(C3)N2C(=O)OC(C)(C)C)cc(C)c1F]

### tert-butyl 3-amino-2-(4-fluoro-3,5-dimethylphenyl)-2,4,5,6,7,8-hexahydro-4,7-epiminocyclohepta[c]pyrazole-9-carboxylate (Scheme B)

### Step 1: tert-butyl 2-cyano-3-oxo-8-azabicyclo[3.2.1]octane-8-carboxylate

To a solution of LDA (5.33 mL, 10.7 mmol) at −78° C. under an atmosphere of nitrogen was added tert-butyl 3-oxo-8-azabicyclo[3.2.1]octane-8-carboxylate (2.00 g, 8.88 mmol) in THF (10 mL). After 25 minutes, the solution containing the above enolate was cannulated into a solution of 4-methylbenzenesulfonyl cyanide (3.22 g, 17.8 mmol) in THF (5.00 mL) at −78° C. The resultant mixture was stirred at −78° C. for 30 min. Then, the reaction mixture was quenched by addition of concentrated ammonium hydroxide (5 mL) and was warmed to rt. The resultant mixture was neutralized with 3 N HC aq. solution (adjusting the pH of the mixture to approx. 6 to 7). The mixture was then extracted with EtOAc, washed with brine, dried over Na₂SO₄, filtered, and concentrated under reduced pressure. The crude residue was purified by SiO₂ chromatography (0 to 50% EtOAc/hexanes) to provide the title compound. MS (ESI) m/z: 250.9 [M+H]+.

### Step 2: tert-butyl 3-amino-2-(4-fluoro-3,5-dimethylphenyl)-2,4,5,6,7,8-hexahydro-4,7-epiminocyclohepta[c]pyrazole-9-carboxylate

To a solution of tert-butyl 2-cyano-3-oxo-8-azabicyclo[3.2.1]octane-8-carboxylate (100 mg, 0.40 mmol) in EtOH (10 mL) was added (4-fluoro-3,5-dimethylphenyl)hydrazine (80 mg, 0.52 mmol), the resulting mixture was stirred at 90° C. for 3 h. Then, the reaction mixture was poured into water (20 mL), the pH of the mixture was adjusted to 9 by the addition of aqueous NaOH, and extracted with EtOAc (3×20 mL). The combined organic layers were dried over Na₂SO₄, filtered, and concentrated under reduced pressure to provide the title compound. MS (ESI) m/z: 387.0 [M+H]+. The following intermediates in table B were prepared according to scheme B using the procedures outlined in the synthesis of intermediates B1.

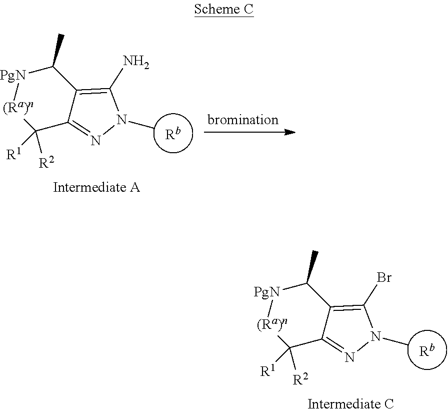

Intermediate C is prepared according to Scheme C via nucleophilic aromatic substitution of intermediate A by treatment with sodium nitrite or isoamyl nitrite (or other diazotization reagents) and bromide salts such as Cu(I)Br and to yield intermediate C.

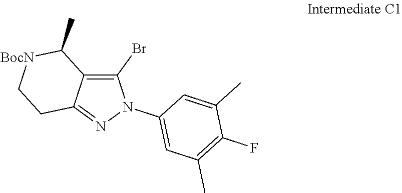 [SMILES: Cc1cc(-n2nc3c(c2Br)[C@H](C)N(C(=O)OC(C)(C)C)CC3)cc(C)c1F]

### tert-butyl (S)-3-bromo-2-(4-fluoro-3,5-dimethylphenyl)-4-methyl-2,4,6,7-tetrahydro-5H-pyrazolo[4,3-c]pyridine-5-carboxylate (Scheme C)

### Step 1: tert-butyl (S)-3-bromo-2-(4-fluoro-3,5-dimethylphenyl)-4-methyl-2,4,6,7-tetrahydro-5H-pyrazolo[4,3-c]pyridine-5-carboxylate

To a solution of tert-butyl (S)-3-amino-2-(4-fluoro-3,5-dimethylphenyl)-4-methyl-2,4,6,7-tetrahydro-5H-pyrazolo[4,3-c]pyridine-5-carboxylate in MeCN (1.40 L) at room temperature under an inert (nitrogen) atmosphere was added copper(I) bromide (188 g, 1310 mmol) then isoamyl nitrite (153 g, 1310 mmol) drop-wise. The resulting mixture was stirred for 1 h at room temperature. The solid precipitate was filtered and washed with MeCN (2×100 mL). The resulting mixture was concentrated under reduced pressure, then purified by silica gel chromatography (petroleum ether/EtOAc: 5/1) to provide the title compound. 1H NMR: (400 MHz, DMSO-d6) δ 7.27 (d, J=6.3 Hz, 2H), 5.02 (d, J=33.2 Hz, 1H), 4.21 (d, J=27.9 Hz, 1H), 3.14 (s, 1H), 2.76-2.52 (m, 2H), 2.28 (d, J=2.2 Hz, 6H), 1.44 (s, 9H), 1.36 (d, J=6.6 Hz, 3H). MS: 43 8.1, 440.1. The following intermediates in table C were prepared according to scheme C using the procedures outlined in the synthesis of intermediate C1.

TABLE C

| | | | MS |
|---|---|---|---|
| Intermediate | Structure | Name | (M + 1) |
| C2 | 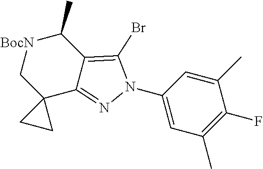 [SMILES: Cc1cc(-n2nc3c(c2Br)[C@H](C)N(C(=O)OC(C)(C)C)CC32CC2)cc(C)c1F] | tert-butyl (S)-3′-bromo-2′-(4-fluoro-3,5- dimethylphenyl)-4′-methyl-2′,4′- dihydrospiro[cyclopropane-1,7′- pyrazolo[4,3-c]pyridine]-5′(6′H)- carboxylate | 464.4, 466.4 |
| C3 | 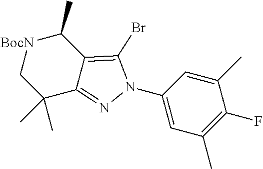 [SMILES: Cc1cc(-n2nc3c(c2Br)[C@H](C)N(C(=O)OC(C)(C)C)CC3(C)C)cc(C)c1F] | tert-butyl (S)-3-bromo-2-(4-fluoro-3,5- dimethylphenyl)-4,7,7-trimethyl-2,4,6,7- tetrahydro-5H-pyrazolo[4,3-c]pyridine-5- carboxylate | 466.4, 468.4 |
| C4 | 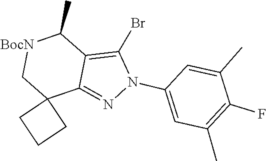 [SMILES: Cc1cc(-n2nc3c(c2Br)[C@H](C)N(C(=O)OC(C)(C)C)CC32CCC2)cc(C)c1F] | tert-butyl (S)-3′-bromo-2′-(4-fluoro-3,5- dimethylphenyl)-4′-methyl-2′,4′- dihydrospiro[cyclobutane-1,7′-pyrazolo[4,3- c]pyridine]-5′(6′H)-carboxylate | 478.0, 480.0 |
| C5 | 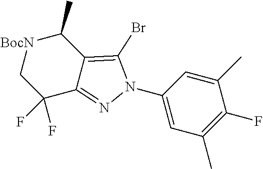 [SMILES: Cc1cc(-n2nc3c(c2Br)[C@H](C)N(C(=O)OC(C)(C)C)CC3(F)F)cc(C)c1F] | tert-butyl (S)-3-bromo-7,7-difluoro-2-(4- fluoro-3,5-dimethylphenyl)-4-methyl- 2,4,6,7-tetrahydro-5H-pyrazolo[4,3- c]pyridine-5-carboxylate | 473.8, 475.8 |
| VB-2 | 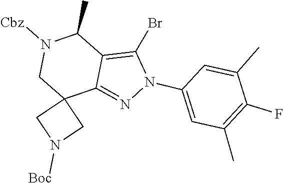 [SMILES: Cc1cc(-n2nc3c(c2Br)[C@H](C)N(C(=O)OCc2ccccc2)CC32CN(C(=O)OC(C)(C)C)C2)cc(C)c1F] | 5′-benzyl 1-(tert-butyl)(S)-3′-bromo-2′-(4- fluoro-3,5-dimethylphenyl)-4′-methyl-2′,4′- dihydrospiro[azetidine-3,7′-pyrazolo[4,3- c]pyridine]-1,5′(6′H)-dicarboxylate | 635.0, 637.0 |
| BW-2A | 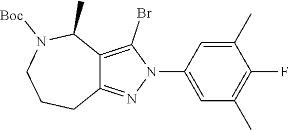 [SMILES: Cc1cc(-n2nc3c(c2Br)[C@H](C)N(C(=O)OC(C)(C)C)CCC3)cc(C)c1F] | tert-butyl (S)-3-bromo-2-(4-fluoro-3,5- dimethylphenyl)-4-methyl-2,6,7,8- tetrahydropyrazolo[4,3-c]azepine-5(4H)- carboxylate | 452.2, 454.2 |
| BW-2B | 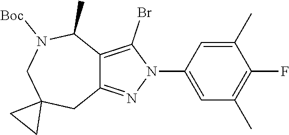 [SMILES: Cc1cc(-n2nc3c(c2Br)[C@H](C)N(C(=O)OC(C)(C)C)CC2(CC2)C3)cc(C)c1F] | tert-butyl (S)-3′-bromo-2′-(4-fluoro-3,5- dimethylphenyl)-4′-methyl-2′,8′-dihydro- 4′H-spiro[cyclopropane-1,7′-pyrazolo[4,3- c]azepine]-5′(6′H)-carboxylate | 478.0, 480.0 |
| BW-2C | 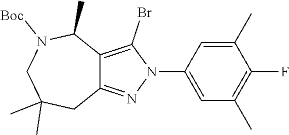 [SMILES: Cc1cc(-n2nc3c(c2Br)[C@H](C)N(C(=O)OC(C)(C)C)CC(C)(C)C3)cc(C)c1F] | tert-butyl (S)-3-bromo-2-(4-fluoro-3,5- dimethylphenyl)-4,7,7-trimethyl-2,6,7,8- tetrahydropyrazolo[4,3-c]azepine-5(4H)- carboxylate | 480.0, 482.0 |
| MU-C1 | 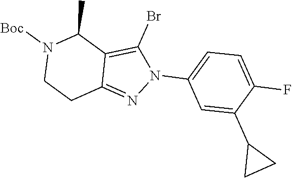 [SMILES: C[C@H]1c2c(nn(-c3ccc(F)c(C4CC4)c3)c2Br)CCN1C(=O)OC(C)(C)C] | tert-butyl (S)-3-bromo-2-(3-cyclopropyl-4- fluorophenyl)-4-methyl-2,4,6,7-tetrahydro- 5H-pyrazolo[4,3-c]pyridine-5-carboxylate | 450.0, 452.2 |
| MU-C2 | 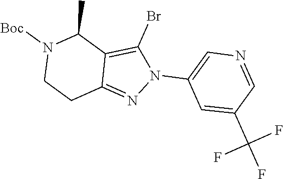 [SMILES: C[C@H]1c2c(nn(-c3cncc(C(F)(F)F)c3)c2Br)CCN1C(=O)OC(C)(C)C] | tert-butyl (S)-3-bromo-4-methyl-2-(5- (trifluoromethyl)pyridin-3-yl)-2,4,6,7- tetrahydro-5H-pyrazolo[4,3-c]pyridine-5- carboxylate | 461.1, 463.0 |
| MU-C3 | 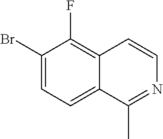 | 6-bromo-5-fluoro-1-methylisoquinoline | 241.9 |

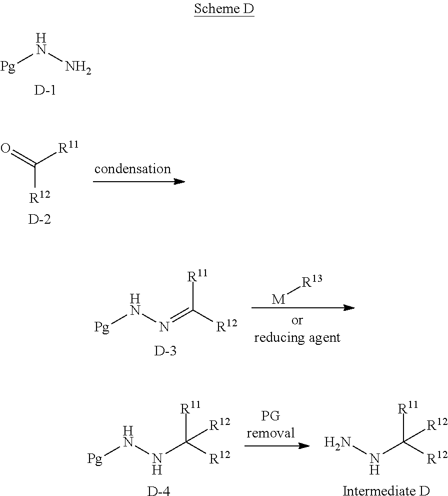

Intermediate D is prepared according to Scheme C via condensation of hydrazine D-1 with ketone or aldehyde D-2 to yield intermediate D-3. Subsequently, treatment of D-3 with organometallic reagents (e.g., RLi or RMgBr) or hydride reagents (e.g., NaCNBH₃ or NaBH₄) synthesized D-4. Removal of the protecting group (e.g., acidic conditions or Pd-catalyzed hydrogenolysis) provides intermediate D.

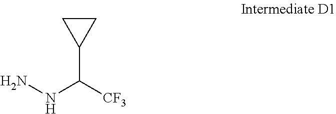

### (1-cyclopropyl-2,2,2-trifluoroethyl)hydrazine (scheme D)

### Step 1: N′-(2,2,2-trifluoroethylidene)benzohydrazide

A solution of benzohydrazide (10 g, 73 mmol) and 2,2,2-trifluoroethane-1,1-diol (8.5 g, 73 mmol) in MeOH (37 mL) was placed in a sealed tube. After addition of freshly dried 4A molecular sieves (10 g), the tube was sealed and heated in an oil bath to 75° C. for 24 h. Upon reaction completion, the mixture was cooled to rt, filtered, and washed with a portion of MeOH (3×50 mL). The combined filtrate was concentrated under reduced pressure. The crude mixture was then purified by silica gel chromatography (0 to 45% EtOAc/Pet.ether) to provide the title compound. LCMS (ESI) m/z: 217.1 [M+H]+.

### Step 2: N′-(1-cyclopropyl-2,2,2-trifluoroethyl)benzohydrazide

To a solution of N′-(2,2,2-trifluoroethylidene)benzohydrazide (2 g, 9.3 mmol) in THF (60 mL) at 0° C. was added a cyclopropylmagnesium bromide (0.5 M in THF) (40 mL, 20 mmol). The reaction mixture was stirred at 10° C. for 16 h. Upon reaction completion, the mixture was quenched with a saturated aqueous solution of NH₄Cl (50 mL) and extracted with EtOAc (3×50 mL), dried over Na₂SO₄, filtered, and concentrated under reduced pressure. The crude residue was purified by silica gel chromatography (9% EtOAc/Pet.ether) to provide the title compound. LCMS (ESI) m/z: 259.2 [M+H]+.

### Step 3: (1-cyclopropyl-2,2,2-trifluoroethyl)hydrazine

To a solution of hydrochloric acid (12 M in water) (50 mL) was added N′-(1-cyclopropyl-2,2,2-trifluoroethyl)benzohydrazide (5.6 g, 22 mmol). The reaction mixture was stirred at 80° C. for 16 h, then concentrated under reduced pressure. The crude residue was suspended in EtOAc, and the solid precipitate was filtered and washed with EtOAc to provide the title compound. ¹H NMR (400 MHz, MeOD) δ=2.85 (qd, J=6.6, 9.8 Hz, 1H), 1.05-0.91 (m, 1H), 0.82-0.74 (m, 2H), 0.60-0.50 (m, 2H).

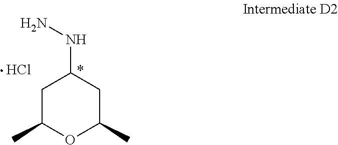

### ((2R,4r,6S)-2,6-dimethyltetrahydro-2H-pyran-4-yl)hydrazine hydrochloride and ((2R,4s,6S)-2,6-dimethyltetrahydro-2H-pyran-4-yl)hydrazine hydrochloride (scheme D)

### Step 1: tert-butyl 2-(2,6-dimethyltetrahydro-4H-pyran-4-vlidene)hydrazine-1-carboxylate

To a stirred solution of 2,6-dimethyltetrahydro-4H-pyran-4-one (500 mg, 3.9 mmol) in methanol (3.9 mL) was added tert-butyl hydrazinecarboxylate (516 mg, 3.9 mmol) at rt. The reaction mixture was stirred at rt for 19 hours. The reaction was concentrated to afford the title compound that was used directly in the next step. LCMS (ESI) m/z: 187.1 [M−tBu].

### Step 2: tert-butyl 2-((2R,4r,6S)-2,6-dimethyltetrahydro-2H-pyran-4-yl)hydrazine-1-carboxylate and tert-butyl 2-((2R,4s,6S)-2,6-dimethyltetrahydro-2H-pyran-4-yl)hydrazine-1-carboxylate

To a stirred solution of tert-butyl 2-(2,6-dimethyltetrahydro-4H-pyran-4-ylidene)hydrazine-1-carboxylate (945 mg, 3.9 mmol) in water (3.9 mL) and acetic acid (3.9 mL) was added sodium cyanoborohydride (270 mg, 4.29 mmol) at rt. The reaction mixture was stirred at rt for 18 hours. The mixture was basified with 4M aq NaOH (10 mL) and extracted with DCM (2×10 mL). The combined organic layers were washed with saturated aqueous sodium bicarbonate (10 mL), dried with magnesium sulfate, filtered and concentrated under reduced pressure to afford the title compound that was used in the next step without purification. LCMS (ESI) m/z: 189.2 [M−tBu].

### Step 3: ((2R,4r,6S)-2,6-dimethyltetrahydro-2H-pyran-4-yl)hydrazine hydrochloride and ((2R,4s,6S)-2,6-dimethyltetrahydro-2H-pyran-4-yl)hydrazine hydrochloride

To a stirred solution of tert-butyl 2-(2,6-dimethyltetrahydro-2H-pyran-4-yl)hydrazine-1-carboxylate (950 mg, 3.9 mmol) in 1,4-dioxane (4.0 mL) was added HCl in dioxane (4M, 4.86 mL, 19.4 mmol) at room temperature. The reaction mixture was stirred at room temperature for 3 h. Additional HCl in dioxane (4M, 4.86 mL, 19.4 mmol) was added and the reaction stirred at rt for 18 h. The mixture was concentrated under reduced pressure to afford the title compound that was used in the next step without purification. ¹H-NMR (400 MHz, CDCl₃, 2:1 mixture of diastereomers) 6 7.60 (4H, br s, both isomers, —NHNH₃Cl), [3.99 (0.66H, m, major isomer, H^{a}), 3.77 (0.33H, m, minor isomer, H^{a})], 3.66-3.43 (2H, m, H^{b}), 2.26-2.11 (2H, m, H^{c}), 1.61-1.39 (2H, m, H^{d}), [1.26 (4H, d, J=5.6 Hz, major isomer, 2×CH₃), 1.20 (2H, d, J=5.9 Hz, minor isomer, 2×CH₃)]. MS: 145.2. The following intermediates in table D were prepared according to scheme D using the procedures outlined in the synthesis of intermediates D1 or D2.

TABLE D

| Intermediate | Structure | Name | MS (M + 1) |
|---|---|---|---|
| D3 | 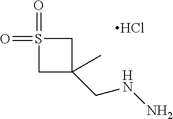 | 3-(hydrazineylmethyl)-3- methylthietane 1,1-dioxide hydrochloride | 165.1 |
| D4 | 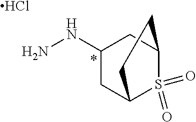 [SMILES: Cl ; NNC1C[C@H]2CC[C@@H](C1)S2(=O)=O] | (1R,3r,5S)-3-hydrazineyl-8- thiazbicyclo[3.2.1]octane 8,8-dioxide and (1R,3s,5S)-3-hydrazineyl-8- thiabicyclo[3.2.1]octane 8,8-dioxide | 191.1 |

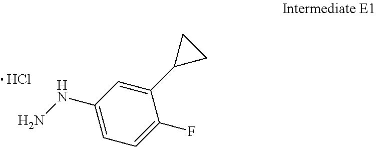

### (3-cyclopropyl-4-fluorophenyl)hydrazine

3-cyclopropyl-4-fluoroaniline (3 g, 19.8 mmol) was added to concentrated HCl (30 mL) at 0° C., and the mixture was stirred for 10 min. Sodium nitrite (1.37 g, 19.8 mmol) in water (10 mL) was added slowly to over a period of 15 min to this mixture. Then, after stirring for 2 h, tin(II)chloride dihydrate (8.96 g, 39.7 mmol) in concentrated HCl (20 mL) was added drop-wise over a period of 10 min. The resulting mixture was stirred for 12 h at rt. The reaction mixture was filtered and washed with concentrated HCl (20 mL) followed by Et₂O (50 mL) and dried under reduced pressure to afford the title compound. ¹H NMR (DMSO-d6, 400 MHz): δ 10.31 (3H, s), 6.98-7.08 (1H, m), 6.77-6.80 (1H, m), 6.71 (1H, d, J=6.3 Hz), 1.94-1.99 (1H, m), 0.94 (2H, m), 0.64-0.74 (2H, m).

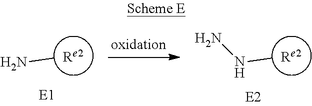

Intermediate E2 is prepared through an oxidation nitration using sodium nitrite for example in concentrated HCl. R^{e2} is aryl, heteroaryl, linear alkyl, cycloalkyl or saturated heterocycle.

The following intermediates in table E were prepared according to scheme E using the procedures outlined in the synthesis of intermediate E2.

TABLE E

| Intermediate | Structure | Name | MS (M + 1) |
|---|---|---|---|
| RJ-IA | 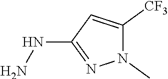 | 3-hydraziney1-1-methyl-5- (trifluoromethyl)-1H-pyrazole | 181.2 |

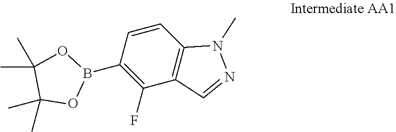

### 4-fluoro-1-methyl-5-(4,4,5,5-tetramethyl-1,3,2-dioxaborolan-2-yl)-1H-indazole

To a mixture of bis(pinacolato)diboron (670 mg, 2.6 mmol), 5-bromo-4-fluoro-1-methyl-1H-indazole (300 mg, 1.3 mmol) and potassium acetate (390 mg, 3.9 mmol) in dioxane (3.0 mL) was added PdCl₂(dppf) (96 mg, 0.13 mmol). The mixture was stirred at 90° C. for 12 h under an atmosphere of N₂. Upon reaction completion, the mixture was filtered and then quenched with H₂O (10 mL) and extracted with EtOAc (3×10 mL). The combined organic layers were washed with brine (20 mL), dried over Na₂SO₄, filtered, and concentrated under reduced pressure. The crude residue was purified by prep-HPLC (40% to 70% MeCN/H₂O+0.1 TFA gradient) to provide the title compound. MS (ESI) m/z: 277.3 [M+H]+.

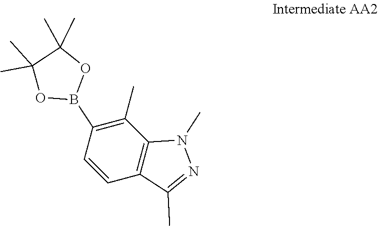

### 1,3,7-trimethyl-6-(4,4,5,5-tetramethyl-1,3,2-dioxaborolan-2-yl)-1H-indazole

The title compound was made in a similar fashion as Intermediate AA1. MS (ESI) m/z: 287.2 [M+H]+.

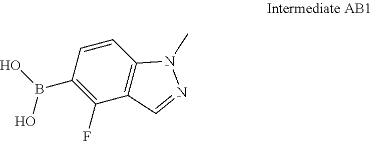

### (4-fluoro-1-methyl-1H-indazol-5-yl)boronic acid

A solution of 5-bromo-4-fluoro-1-methyl-1H-indazole (1.5 g, 6.5 mmol) in THF (35 mL) was cooled to −78° C. Then a solution of nBuLi (0.46 g, 2.3 molar, 7.2 mmol) in hexane was added dropwise. The reaction mixture was stirred at −78° C. Then, trimethyl borate (1.0 g, 9.8 mmol) was added at −60° C. After warming to rt, the resulting mixture was quenched by the addition of aq. citric acid (1M) and stirred for 30 min. The pH of the aqueous layer was adjusted to pH 5 and extracted with EtOAc (3×40 mL). The combined organic layers were dried over sodium sulfate and concentrated under reduced pressure to afford the title compound. MS (ESI) m/z: 195.1 [M+H]+.

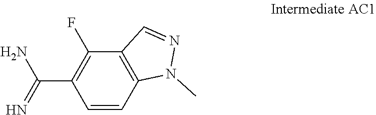

### 4-fluoro-1-methyl-1H-indazole-5-carboximidamide

### Step 1: 4-fluoro-1-methyl-1H-indazole-5-carbonitrile

To a solution of 5-bromo-4-fluoro-1-methyl-1H-indazole (100 mg, 0.44 mmol), potassium ferrocyanide (40 mg, 0.11 mmol), sodium carbonate (46 mg, 0.44 mmol) in NMP (2.0 mL) was added chloro[(tri-tert-butylphosphine)-2-(2-aminobiphenyl)]palladium(II) (22 mg, 0.044 mmol). The resulting mixture was stirred at 80° C. for 18 h. Upon reaction completion, the mixture was quenched with H₂O (10 mL) and extracted with EtOAc (3×5 mL). The combined organic extracts were washed with brine (20 mL), dried over Na₂SO₄, filtered, and concentrated under reduced pressure. The crude residue was purified by prep-HPLC (27% to 47% MeCN/H₂O+0.1 TFA gradient) to provide the title compound. MS (ESI) m/z 176.3.

### Step 2: 4-fluoro-1-methyl-1H-indazole-5-carboximidamide

To a solution of 4-fluoro-1-methyl-1H-indazole-5-carbonitrile (90 mg, 0.51 mmol) in THF (2.0 mL) at 0° C. was added LiHMDS (2.1 mL, 2.1 mmol, 1M in THF). The reaction mixture was stirred at 25° C. under an atmosphere of N₂ for 16 h. Upon reaction completion, the reaction mixture was quenched with HCl, then concentrated under reduced pressure. The crude residue was purified by prep-HPLC (0% to 20% MeCN/H₂O+0.1 TFA gradient) to provide the title compound. MS (ESI) m/z 192.9.

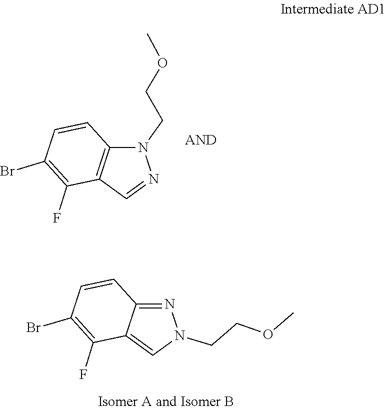

### 5-bromo-4-fluoro-1-(2-methoxyethyl)-1H-indazole and 5-bromo-4-fluoro-2-(2-methoxyethyl)-2H-indazole

Procedure: A vial was charged with 5-bromo-4-fluoro-1H-indazole (90 mg, 0.42 mmol), cesium carbonate (0.27 g, 0.84 mmol), and 1-bromo-2-(2-methoxyethoxy)ethane (0.11 g, 0.63 mmol). N-methyl-2-pyrrolidine (NMP) (3 mL) was added and the mixture was heated to 100° C. in a hot plate for 2 hours. Upon reaction completion, the mixture was cooled to room temperature and diluted with ethyl acetate (20 mL) and extracted with water (3×50 mL), dried over Na₂SO₄, filtered then concentrated under reduced pressure. The crude mixture was purified by silica gel chromatography (0-100% EtOAc/hexanes) to afford the title compounds. Data for isomer A (faster eluting): LCMS (ESI) m/z: 275.2 [M+H]+. ¹H NMR (600 MHz, CDCl₃)¹H NMR (600 MHz, CDCl₃) δ 8.05 (s, 1H), 7.43 (dd, J=8.7, 6.3 Hz, 1H), 7.17 (d, J=8.8 Hz, 1H), 4.52 (t, J=5.3 Hz, 2H), 3.81 (t, J=5.3 Hz, 2H), 3.27 (s, 3H). Data for isomer B (slower eluting): LCMS (ESI) m/z: 275.2 [M+H]+. ¹H NMR (600 MHz, CDCl₃)¹H NMR (600 MHz, CDCl₃) δ 8.09 (s, 1H), 7.38 (d, J=9.1 Hz, 1H), 7.32 (dd, J=8.9, 6.5 Hz, 1H), 4.58 (t, J=5.0 Hz, 2H), 3.87 (t, J=5.0 Hz, 2H), 3.34 (s, 3H). The following intermediates in table AD were prepared using the procedures outlined in the synthesis of intermediate AD1 Isomer A and Isomer B. In cases wherein isomers of the examples were separated via silica gel chromatography, the faster eluting isomer is listed first.

TABLE AD

| | | | MS |
|---|---|---|---|
| Intermediate | Structure | Name | (M + 1) |
| AD2 Isomer A | 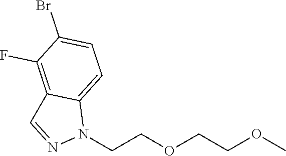 [SMILES: COCCOCCn1ncc2c(F)c(Br)ccc21] | 5-bromo-4-fluoro-1-(2-(2- methoxyethoxy)ethyl)-1H- indazole | 319.2 |
| AD2 Isomer B | 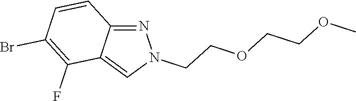 [SMILES: COCCOCCn1cc2c(F)c(Br)ccc2n1] | 5-bromo-4-fluoro-2-(2-(2- methoxyethoxy)ethyl)-2H- indazole | 319.2 |
| AD3 Isomer A | 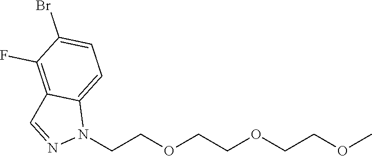 [SMILES: COCCOCCOCCn1ncc2c(F)c(Br)ccc21] | 5-bromo-4-fluoro-1-(2-(2-(2- methoxyethoxy)ethoxy)ethyl)- 1H-indazole | 363.2 |
| AD3 Isomer B | 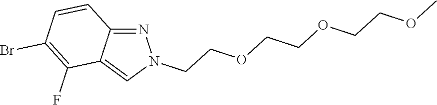 [SMILES: COCCOCCOCCn1cc2c(F)c(Br)ccc2n1] | 5-bromo-4-fluoro-2-(2-(2-(2- methoxyethoxy)ethoxy)ethyl)- 2H-indazole | 363.2 |
| AD4 Isomer A | 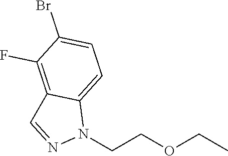 [SMILES: CCOCCn1ncc2c(F)c(Br)ccc21] | 5-bromo-1-(2-ethoxyethyl)-4- fluoro-1H-indazole | 287.2 |
| AD4 Isomer B | 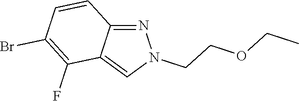 [SMILES: CCOCCn1cc2c(F)c(Br)ccc2n1] | 5-bromo-2-(2-ethoxyethyl)-4- fluoro-2H-indazole | 287.2 |
| AD5 Isomer A | 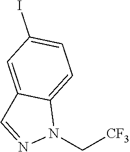 [SMILES: FC(F)(F)Cn1ncc2cc(I)ccc21] | 5-iodo-1-(2,2,2-trifluoroethyl)- 1H-indazole | 327.2 |
| AD5 Isomer B | 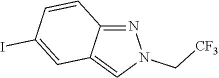 [SMILES: FC(F)(F)Cn1cc2cc(I)ccc2n1] | 5-iodo-2-(2,2,2-trifluoroethyl)- 2H-indazole | 327.2 |

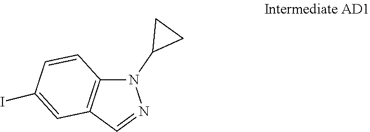 [SMILES: Ic1ccc2c(cnn2C2CC2)c1]

### 1-cyclopropyl-5-iodo-1H-indazole

A mixture of 5-iodo-1H-indazole (2.00 g, 8.20 mmol), cyclopropaneboronic acid (1.41 g, 16.4 mmol), copper diacetate (1.49 g, 8.20 mmol), 2,2′-biyridine (1.28 g, 8.20 mmol) and sodium carbonate (1.74 g, 16.4 mmol) in DCE (41.0 mL) was stirred at 60° C. for 3 h. Then, the mixture was cooled to rt, quenched with aq. NH₄OH and extracted with DCM (2×). The combined organic layers were dried over Na₂SO₄, filtered, and concentrated under reduced pressure. The crude residue was purified by SiO₂ chromatography (0 to 50% EtOAc/Hex) to provide the title compound. ¹H NMR (500 MHz, CD³CN) δ 8.15 (d, J=0.9 Hz, 1H), 7.89 (s, 1H), 7.68 (dd, J=8.8, 1.5 Hz, 1H), 7.52 (d, J=8.8 Hz, 1H), 3.67-3.60 (m, 1H), 1.15 (d, J=5.4 Hz, 4H). MS (ESI) m/z. 285.2 [M+H]+.

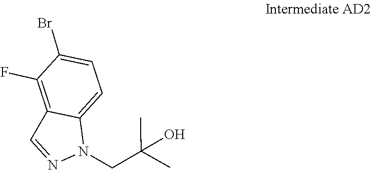 [SMILES: CC(C)(O)Cn1ncc2c(F)c(Br)ccc21]

### 1-(5-bromo-4-fluoro-1H-indazol-1-yl)-2-methylpropan-2-ol

A vial was charged with 5-bromo-4-fluoro-1H-indazole (200 mg, 0.9 mmol), 2,2-dimethyloxirane (134 mg, 1.86 mmol) and potassium carbonate (386 mg, 2.79 mmol. NMP (6 mL) was added to the vial and the reaction mixture was heated to 180° C. for 45 minutes. The mixture was cooled to room temperature, diluted with EtOAc, and extracted with water (3×20 mL), dried over sodium sulfate, filtered then concentrated in vacuo. The crude mixture was purified by silica gel chromatography (0-100% EtOAc/hexanes) to afford the title compound. MS (ESI) m/z: 271.2 [M-OH]+

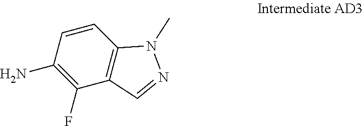

### 4-fluoro-1-methyl-1H-indazol-5-amine

### Step 1: tert-butyl (4-fluoro-1-methyl-1H-indazol-5-yl)carbamate

To a solution of potassium phosphate tribasic (5.0 g, 26 mmol), 5-bromo-4-fluoro-1-methyl-1H-indazole (2.0 g, 8.7 mmol) and tert-butyl carbamate (1.5 g, 13 mmol) in dioxane (29 mL) was added dtbpf-Pd-G3 (400 mg, 0.440 mmol) under an atmosphere of N₂. The reaction mixture was stirred at 100° C. overnight, then cooled to rt and filtered with washing with DCM. The filtrate was concentrated under reduced pressure and the crude residue was purified by silica gel chromatography (0 to 100% EtOAc/Hex) to provide the title compound. MS (ESI) m/z: 266.2 [M+H]+.

### Step 2: 4-fluoro-1-methyl-1H-indazol-5-amine

To a solution of tert-butyl (4-fluoro-1-methyl-1H-indazol-5-yl)carbamate (500 mg, 1.9 mmol) in DCM (1.9 mL) was added 4M HCl (2.4 mL, 9.42 mmol) at rt. After 1 h an additional portion of HCl was added, and the reaction mixture was stirred for 1 h. To the mixture was added Et₂O and the solid precipitate was collected by filtration. The HCl salt was suspended in DCM and washed with 1M aq. NaOH. The layers were separated, and aqueous layer was extracted with DCM (2 x). The combined organic layers were dried over Na₂SO₄, filtered then concentrated under reduced pressure to afford the title compound. ¹H NMR (500 MHz, CD₃CN) δ 7.84 (s, 1H), 7.15 (d, J=8.7 Hz, 1H), 7.02 (t, J=8.4 Hz, 1H), 2.16 (s, 3H). MS (ESI) m/z: 166.2 [M+H]+.

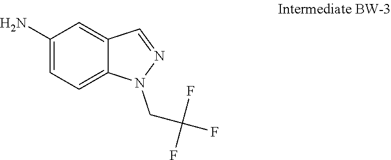 [SMILES: Nc1ccc2c(cnn2CC(F)(F)F)c1]

### Step 1: (1-(2,2,2-trifluoroethyl)-1H-indazol-5-yl)carbamate

To a mixture of 5-iodo-1-(2,2,2-trifluoroethyl)-1H-indazole (250 mg, 770 μmol, 1 equiv), tripotassium phosphate (488 mg, 2.30 mmol), tert-butyl carbamate (135 mg, 1.15 mmol) and BrettPhos Pd G3 (35 mg, 38 μmol) was added 1,4-dioxane (3.0 mL, 0.25 M), and the resulting solution was sparged with argon for 10 min. The vial was then sealed and the mixture heated to 100° C. for 18 h before being cooled, filtered over Celite®, and concentrated in vacuo. The crude residue was purified by flash column chromatography (0-100% EtOAc/hexanes) to give tert-butyl (1-(2,2,2-trifluoroethyl)-1H-indazol-5-yl)carbamate. MS (ESI) m/z: 316.2 [M+H]

### Step 2: 1-(2,2,2-trifluoroethyl)-1H-indazol-5-amine

To tert-butyl (1-(2,2,2-trifluoroethyl)-1H-indazol-5-yl)carbamate (314 mg, 996 μmol, 1 equiv) was added HCl in 1,4-dioxane (4 M,1.2 mL, 5.0 mmol, 5 equiv) and the resulting mixture was stirred at ambient temperature for 18 h. The resulting heterogeneous mixture was then concentrated under reduced pressure before sat. aq. NaHCO₃ (10 mL) and EtOAc (10 mL) were added, and the layers were separated. The aqueous phase was extracted with EtOAc (2×5 mL) and the combined organic layers were dried over Na₂SO₄ and concentrated under reduced pressure to provide 1-(2,2,2-trifluoroethyl)-1H-indazol-5-amine. ¹H NMR (500 MHz, CDCl₃) δ 7.88 (s, 1H), 7.25 (s, 1H), 6.98-6.89 (m, 2H), 4.87 (q, J=8.5 Hz, 2H). ¹⁹F NMR (471 MHz, CDCl₃) δ −70.84 (t, J=8.5 Hz). MS (ESI) m/z: 216.2 [M+H]+

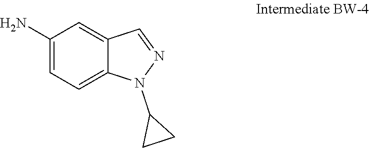 [SMILES: Nc1ccc2c(cnn2C2CC2)c1]

### Step 1: tert-butyl (1-cyclopropyl-1H-indazol-5-yl)carbamate

To a mixture of 1-cyclopropyl-5-iodo-1H-indazole (250 mg, 880 μmol), tripotassium phosphate (560 mg, 2.6 mmol), tert-butyl carbamate (155 mg, 1.32 mmol) and BrettPhos Pd G3 (40 mg, 40 μmol) was added 1,4-dioxane (3.3 mL, 0.25 M) and the resulting solution was sparged with argon for 10 min. The vial was then sealed and the mixture heated to 100° C. for 18 h before being cooled, filtered over Celite®, and concentrated in vacuo. The crude residue was purified by flash column chromatography (0-100% EtOAc/hexanes) to give tert-butyl (1-cyclopropyl-1H-indazol-5-yl)carbamate. MS (ESI) m/z: 274.4 [M+H]+

### Step 2: 1-cyclopropyl-1H-indazol-5-amine

To tert-butyl (1-cyclopropyl-1H-indazol-5-yl)carbamate (179 mg, 655 μmol, 1 equiv) was added HCl in 1,4-dioxane (4 M, 0.8 mL, 3 mmol) and the resulting mixture was stirred at ambient temperature for 18 h. The resulting heterogeneous mixture was then concentrated under reduced pressure before sat. aq. NaHCO₃ (10 mL) and EtOAc (10 mL) were added, and the layers were separated. The aqueous phase was extracted with EtOAc (2×5 mL) and the combined organic layers were dried over Na₂SO₄ and concentrated under reduced pressure to provide titled compound. 1H NMR (500 MHz, CDCl₃) δ 7.73 (s, 1H), 7.42 (d, J=8.7 Hz, 1H), 6.92 (d, J=1.9 Hz, 1H), 6.88 (dd, J=8.7, 2.0 Hz, 1H), 3.52 (tt, J=7.0, 3.6 Hz, 1H), 1.22-1.16 (m, 2H), 1.14-1.08 (m, 2H). MS (ESI) m/z: 174.4 [M+H]⁺

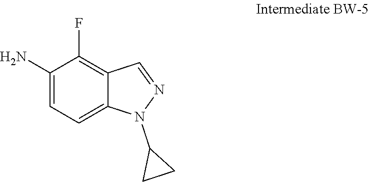 [SMILES: Nc1ccc2c(cnn2C2CC2)c1F]

### Step 1: 5-bromo-1-cyclopropyl-4-fluoro-1H-indazole

To a 100 mL round-bottomed flask was added 5-bromo-4-fluoro-1H-indazole (1.00 g, 31.0 mL, 0.15 molar, 4.65 mmol) copper (II) acetate (845 mg, 4.65 mmol) 2,2′-bipyridine (726 mg, 4.65 mmol) cyclopropylboronic acid (799 mg 9.30 mmol) sodium carbonate (986 mg, 9.30 mmol) 1,2-DCE (30 mL) and heated to 70° C. for 2 h. Then the mixture was filtered, concentrated under reduced pressure, redissolved in EtOAc (30 mL), and washed twice with sat. aq. CuSO₄ (2×10 mL). Then the organic layer was washed with brine, dried with Na₂SO₄, and concentrated. The crude mixture was purified by flash column chromatography (40 g SiO₂ column, 0 to 100% EtOAc, 40 mL/min) to provide 5-bromo-1-cyclopropyl-4-fluoro-1H-indazole. ¹H NMR (500 MHz, CDCl₃) δ 7.98 (s, 1H), 7.46 (dd, J=8.8, 6.2 Hz, 1H), 7.28 (d, J=8.8 Hz, 1H), 3.58 (tt, J=7.0, 3.5 Hz, 1H), 1.26-1.15 (m, 4H). MS (ESI) m/z: 257.0 [M+H]⁺

### Step 2: tert-butyl (1-cyclopropyl-4-fluoro-1H-indazol-5-yl)carbamate

To a mixture of 5-bromo-1-cyclopropyl-4-fluoro-1H-indazole (500 mg, 2 mmol, 1 equiv), tripotassium phosphate (1.25 g, 5.88 mmol), tert-butyl carbamate (344 mg, 2.94 mmol) and BrettPhos Pd G3 (90 mg, 100 μmol) was added 1,4-dioxane (7.3 mL, 0.25 M), and the resulting solution was sparged with argon for 10 min. The vial was then sealed and the mixture heated to 100° C. for 18 h before being cooled, filtered over Celite®, and concentrated in vacuo. The crude residue was purified by flash column chromatography (0-100% EtOAc/hexanes) to provide tert-butyl (1-cyclopropyl-4-fluoro-1H-indazol-5-yl)carbamate. MS (ESI) m/z: 292.2 [M+H]⁺

### Step 3: 1-cyclopropyl-4-fluoro-1H-indazol-5-amine

To tert-butyl (1-cyclopropyl-4-fluoro-1H-indazol-5-yl)carbamate (403 mg, 1.38 mmol) was added HCl in 1,4-dioxane (4 M, 3.5 mL, 14 mmol) and the resulting mixture was stirred at ambient temperature for 18 h. The resulting heterogeneous mixture was then concentrated under reduced pressure before sat. aq. NaHCO₃ (10 mL) and DCM (10 mL) were added, and the layers were separated. The aqueous phase was extracted with DCM (2×5 mL) and the combined organic layers were dried over Na₂SO₄ and concentrated under reduced pressure to provide the titled compound. ¹H NMR (500 MHz, CDCl₃) δ 7.88 (s, 1H), 7.23 (d, J=8.7 Hz, 1H), 7.07 (d, J=8.5 Hz, 1H), 3.56-3.50 (m, 1H), 1.21-1.18 (m, 2H), 1.16-1.11 (m, 2H). ¹⁹F NMR (471 MHz, CDCl₃) δ −140.21. MS (ESI) m/z: 192.2 [M+H]⁺

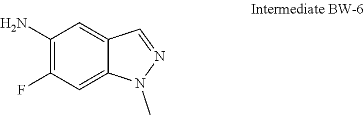

### Step 1: tert-butyl (6-fluoro-1-methyl-1H-indazol-5-yl)carbamate

To a mixture of 5-bromo-6-fluoro-1-methyl-1H-indazole (150 mg, 660 μmol), tripotassium phosphate (417 mg, 1.96 mmol), tert-butyl carbamate (115 mg, 982 μmol) and BrettPhos Pd G3 (30 mg, 30 μmol) was added 1,4-dioxane (2.5 mL, 0.25 M) and the resulting solution was sparged with argon for 10 min. The vial was then sealed and the mixture heated to 100° C. for 18 h before being cooled, filtered over Celite®, and concentrated in vacuo. The crude residue was purified by flash column chromatography (0-100% EtOAc/hexanes) to provide tert-butyl (6-fluoro-1-methyl-1H-indazol-5-yl)carbamate. MS (ESI) m/z: 266.2 [M+H]⁺

### Step 2: 1-cyclopropyl-1H-indazol-5-amine

To tert-butyl (6-fluoro-1-methyl-1H-indazol-5-yl)carbamate (230 mg, 870 μmol, 1 equiv) was added HCl in 1,4-dioxane (4 M, 2.2 mL, 8.7 mmol, 10 equiv) and the resulting mixture was stirred at ambient temperature for 18 h. The resulting heterogeneous mixture was then concentrated under reduced pressure before sat. aq. NaHCO₃ (10 mL) and DCM (10 mL) were added, and the layers were separated. The aqueous phase was extracted with DCM (2×5 mL) and the combined organic layers were dried over Na₂SO₄ and concentrated under reduced pressure to provide title compound. ¹H NMR (500 MHz, CDCl₃) δ 7.79 (s, 1H), 7.05 (d, J=6.7 Hz, 1H), 7.03 (d, J=4.4 Hz, 1H), 4.00 (s, 3H). ¹⁹F NMR (471 MHz, CDCl₃) δ −130.65 (dd, J=10.6, 8.5 Hz). MS (ESI) m/z: 166.2 [M+H]⁺

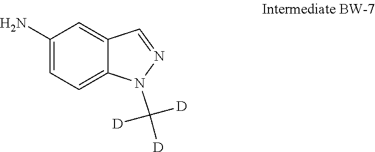 [SMILES: [2H]C([2H])([2H])n1ncc2cc(N)ccc21]

### Step 1: tert-butyl (1-(methyl-d3)-1H-indazol-5-yl)carbamate

To a mixture of 5-bromo-1-(methyl-d3)-1H-indazole (150 mg, 0.70 mmol), tripotassium phosphate (446 mg, 2.10 mmol), tert-butyl carbamate (123 mg, 1.05 μmol) and BrettPhos Pd G3 (32 mg, 35 μmol) was added 1,4-dioxane (2.6 mL, 0.25 M) and the resulting solution was sparged with argon for 10 min. The vial was then sealed and the mixture heated to 100° C. for 18 h before being cooled, filtered over Celite®, and concentrated in vacuo. The crude residue was purified by flash column chromatography (0-100% EtOAc/hexanes) to provide tert-butyl (1-(methyl-d3)-1H-indazol-5-yl)carbamate. MS (ESI) m/z: 251.2 [M+H]+

### Step 2: 1-(methyl-d3)-1H-indazol-5-amine

To tert-butyl (1-(methyl-d3)-1H-indazol-5-yl)carbamate (179 mg, 715 μmol) was added HCl in 1,4-dioxane (4 M, 0.90 mL, 3.6 mmol) and the resulting mixture was stirred at ambient temperature for 18 h. The resulting heterogeneous mixture was then concentrated under reduced pressure before sat. aq. NaHCO₃ (10 mL) and EtOAc (10 mL) were added, and the layers were separated. The aqueous phase was extracted with EtOAc (2×5 mL) and the combined organic layers were dried over Na₂SO₄ and concentrated under reduced pressure to give 1-(methyl-d3)-1H-indazol-5-amine as a solid. 1H NMR (500 MHz, CDCl₃) δ 7.77 (s, 1H), 7.22 (d, J=8.7 Hz, 1H), 6.93 (d, J=1.5 Hz, 1H), 6.88 (dd, J=8.7, 2.1 Hz, 1H). MS (ESI) m/z: 151.2 [M+H]+

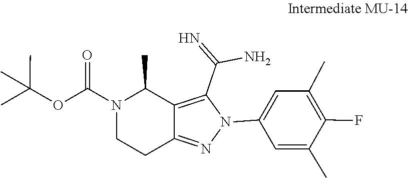 [SMILES: Cc1cc(-n2nc3c(c2C(=N)N)[C@H](C)N(C(=O)OC(C)(C)C)CC3)cc(C)c1F]

### tert-butyl (S)-3-carbamimidoyl-2-(4-fluoro-3,5-dimethylphenyl)-4-methyl-2,4,6,7-tetrahydro-5H-pyrazolo[4,3-c]pyridine-5-carboxylate

### Step 1: tert-butyl (S)-3-cyano-2-(4-fluoro-3,5-dimethylphenyl)-4-methyl-2,4,6,7-tetrahydro-5H-pyrazolo[4,3-c]pyridine-5-carboxylate

To a solution of tert-butyl (S)-3-bromo-2-(4-fluoro-3,5-dimethylphenyl)-4-methyl-2,4,6,7-tetrahydro-5H-pyrazolo[4,3-c]pyridine-5-carboxylate (200.00 mg, 456.27 mol), potassium ferricyanide (37.556 mg, 20.06 μL, 114.07 mol), Na₂CO₃ (48.360 mg, 456.27 mol) in NMP (5 mL) was added XPhos Pd G2 (35.900 mg, 0.1 Eq, 45.627 mol) in a nitrogen filled glovebox, and the resulting mixture was stirred at 100° C. for 16 h. The mixture was diluted with H₂O (15 mL) and extracted with EtOAc (10 mL, 3×). The combined organic fraction was washed with brine (20 mL), dried over anhydrous Na₂SO₄, filtered and concentrated. The resulting material was purified by prep-HPLC (Column Y: MC-Actus Triart C18 150*30 mm*5 m. Mobile phase A: water (containing water (0.10% TFA)-ACN), mobile phase B: acetonitrile. Gradient: 27%˜47% B, 0-10 min. Flow Rate: 40 mL/min) followed by lyophilization. m/z: 385.2 [M+H]⁺.

### Step 2: tert-butyl (S)-3-carbamimidoyl-2-(4-fluoro-3,5-dimethylphenyl)-4-methyl-2,4,6,7-tetrahydro-5H-pyrazolo[4,3-c]pyridine-5-carboxylate

To a solution of tert-butyl (S)-3-cyano-2-(4-fluoro-3,5-dimethylphenyl)-4-methyl-2,4,6,7-tetrahydro-5H-pyrazolo[4,3-c]pyridine-5-carboxylate (340.50 mg, 885.66 mol) in THF (5 mL) was added lithium bis(trimethylsilyl)amide (889.18 mg, 5.31 mL, 1 molar, 5.31 mmol) at 0° C. The reaction mixture was stirred at 30° C. under N₂ for 16 hours. The reaction solution was quenched with aq. HCl (1M), then was concentrated under reduced pressure. The residue was purified by prep-HPLC (Column: YMC-Actus Triart C18 150*30 mm*5 m. Mobile phase A: water (containing Water (water (0.1% TFA)-ACN), mobile phase B: acetonitrile. Gradient: 20%˜50% B, 0˜11 min. Flow Rate: 40 mL/min) followed by lyophilization. ¹H NMR (400 MHz, chloroform-d) δ ppm 2.65 (s, 3H) 6.62 (dd, J=7.51, 1.79 Hz, 1H) 7.30 (s, 1H) 7.50-7.66 (m, 2 H), m/z: 402.1 [M+H]⁺.

The following intermediates in table MU-Ti were prepared using the procedures outlined in the synthesis of intermediate MU-14.

TABLE MU-T1

| Intermediate | Structure | Name | MS (M + H) |
|---|---|---|---|
| MU-15 | 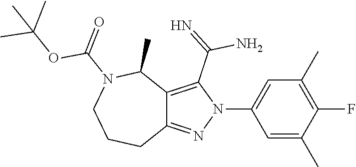 [SMILES: Cc1cc(-n2nc3c(c2C(=N)N)[C@H](C)N(C(=O)OC(C)(C)C)CCC3)cc(C)c1F] | tert-butyl (S)-3-carbamimidoyl-2-(4- fluoro-3,5-dimethylphenyl)-4-methyl- 2,6,7,8-tetrahydropyrazolo[4,3- c]azepine-5(4H)-carboxylate | 416.2 |

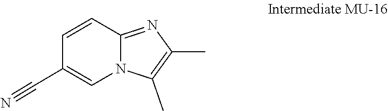

### 2,3-dimethylimidazo[1,2-a]pyridine-6-carbonitrile

To a solution of 6-aminonicotinonitrile (400 mg, 3.36 mmol) in Ethanol (10 mL) was added 3-bromobutan-2-one (2.08 g, 13.8 mmol) at 25° C. Then the reaction mixture was stirred at 100° C. for 16 h. The mixture was concentrated in vacuo and the residue was diluted with water (20 mL) and DCM (30 mL). The resulting mixture was adjusted to pH=10 with NaOH (2M) aqueous solution, then extracted with DCM (30 mL×2). The combined organic layers were dried over anhydrous Na₂SO₄, filtered, and concentrated in vacuo. The residue was purified by silica gel column chromatography (EtOAc/Petroleum ether (v/v)=1/10 to 1/5 to 1/2). ¹H NMR (400 MHz, acetonitrile-d₃) δ=8.57 (s, 1H), 7.54-7.44 (m, 1H), 7.34-7.19 (m, 1H), 2.43 (s, 3H), 2.38 (s, 3H).

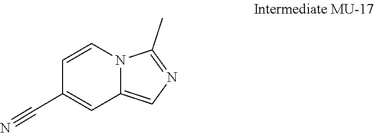

### 3-methylimidazo[1,5-a]pyridine-7-carbonitrile

### Step 1: 7-bromo-3-methylimidazo[1,5-a]pyridine

To a solution of (4-bromopyridin-2-yl)methanamine (1000 mg, 5.35 mmol) in acetic anhydride (3 g, 3 mL, 0.03 mol) was added Ts-OH (1.017 g, 5.346 mmol) at 25° C. Then the reaction mixture was stirred at 100° C. for 16 hours. The mixture was allowed to cool to room temperature, concentrated and poured into water (30 mL). The pH was adjusted pH to 7 with 20% NaOH, and the mixture extracted with dichloromethane (20 mL, ×3). The organic phase was dried over anhydrous sodium sulfate, concentrated, and the crude product was purified by column chromatography with (EA:PE=0:1). m/z: 211.1 [M+H]⁺.

### Step 2: 3-methylimidazo[1,5-a]pyridine-7-carbonitrile

A solution of zinc cyanide (0.46 g, 0.25 mL, 3.9 mmol), tetrakis(triphenylphosphine) palladium(0) (109.50 mg, 94.760 mol), 7-bromo-3-methylimidazo[1,5-a]pyridine (200.00 mg, 947.60 mol) in DMF (3 mL) in a nitrogen filled glovebox, was stirred at 100° C. under N₂ for 16 hours. The mixture was diluted with H₂O (15 mL) and extracted with EtOAc (10 mL, 3×). The combined organic extracts were washed with brine (20 mL), dried over anhydrous Na₂SO₄, filtered, and concentrated under reduced pressure. The pH of the aqueous phase was adjusted 11 with NaOH, then zinc cyanide was quenched with sodium hypochlorite (aq). The residue was purified by prep-TLC(SiO₂, Pet.ether:EtOAc=0:1), m/z: 158.1 [M+H]⁺.

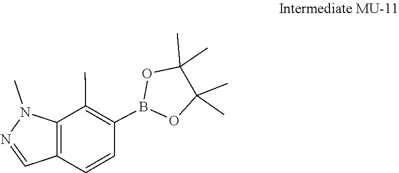 [SMILES: Cc1c(B2OC(C)(C)C(C)(C)O2)ccc2cnn(C)c12]

### 1,7-dimethyl-6-(4,4,5,5-tetramethyl-1,3,2-dioxaborolan-2-yl)-1H-indazole

6-bromo-1,7-dimethyl-1H-indazole (1.72 g, 7.76 mmol), B₂Pin₂ (2.32 g, 9.19 mmol), potassium acetate (1.50 g, 15.32 mmol) and 1,4-dioxane (27.35 mL) were added to a flame dried reaction tube. The mixture was purged with argon for 5 minutes after which PdCl₂(dppf) (168.1 mg, 229.8 mol) was added, and argon purging was continued for another 5 minutes. The mixture was sealed and placed in an oil bath which was preheated to 95° C. and stirred for 18 hours. The reaction mixture was cooled to room temperature and poured into aq. sat. NaHCO₃ (100 mL). The mixture was extracted using EtOAc (2×100 mL) and the combined organic phases were dried over Na₂SO₄, filtered, and concentrated. The resulting material was purified using flash column chromatography (80 g silica, DCM injection) eluting with a gradient from heptane:DCM 8:2 to DCM. ¹H NMR (400 MHz, CDCl₃) δ 7.88 (s, 1H), 7.51 (d, J=8.2 Hz, 1H), 7.46 (d, J=8.1 Hz, 1H), 4.37 (s, 3H), 3.01 (s, 3H), 1.38 (s, 12H). MS (ESI) m/z: 273.0 [M+H]⁺.

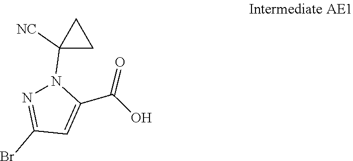 [SMILES: N#CC1(n2nc(Br)cc2C(=O)O)CC1]

### 3-bromo-1-(1-cyanocyclopropyl)-1H-pyrazole-5-carboxylic acid

### Step 1: methyl 3-bromo-1-(cyanomethyl)-1H-pyrazole-5-carboxylate

A mixture of methyl 5-bromo-1H-pyrazole-3-carboxylate (15.5 g, 75.6 mmol), cesium carbonate (61.6 g, 189 mmol) and 2-bromoacetonitrile (18.1 g, 151 mmol) in MeCN (180 mL) was stirred at rt for 3 h. Then, the mixture was filtered through Celite® and washed with CH₃CN (100 ml) and EtOAc (100 mL). The filtrate was concentrated under reduced pressure. The crude material was purified by SiO₂ chromatography (0 to 60% EtOAc/Hex) to afford the title compound. ¹H NMR (400 MHz, CDCl₃): δ 6.91 (s; 1 H); 5.48 (s; 2 H); 3.95 (s; 3 H). MS (ESI) m/z: 244.0, 246.0 [M+H]+.

### Step 2: methyl 3-bromo-1-(1-cyanocyclopropyl)-1H-pyrazole-5-carboxylate

Sodium hydride (369 mg, 60% wt, 9.22 mmol) was added with stirring under an atmosphere of Ar to DMSO (10 mL) maintained at rt by use of a water bath. The resulting suspension was stirred for 10 min before the addition of a solution of methyl 3-bromo-1-(cyanomethyl)-1H-pyrazole-5-carboxylate (500 mg, 2.05 mmol) and 1-bromo-2-chloro-ethane (735 mg, 5.12 mmol) in DMSO (6.0 mL) slowly (via drop-wise addition over a period of 10-15 min (few pieces of ice was added to water bath to keep the temperature below room temperature during the addition process). The reaction mixture was stirred for 1.5 h in the water bath. Then, sat. aq. NH₄Cl (32 mL) was added slowly and the resulting mixture was stirred for 15 min. The mixture was extracted with EtOAc (4×40 mL). The combined organic layers were washed with brine, dried over Na₂SO₄, filter and concentrated under reduced pressure. The crude residue was purified by SiO₂ chromatography (0-50% EtOAc/Hex) to provide the title compound. ¹H NMR (400 MHz, CDCl₃): δ 6.89 (s; 1 H); 3.96 (s; 3 H); 1.93 (t; J=5.68 Hz; 2 H); 1.86 (t; J=5.65 Hz; 2 H).

### Step 3: 3-bromo-1-(1-cyanocyclopropyl)-1H-pyrazole-5-carboxylic acid

A mixture of methyl 3-bromo-1-(1-cyanocyclopropyl)-1H-pyrazole-5-carboxylate (300 mg, 1.11 mmol) in THF (10 mL) was added aq sodium hydroxide (2.22 mL, 1 molar, 2.22 mmol) at 23° C. Then, the reaction mixture was stirred at 23° C. for 1 h. Then, the mixture was concentrated under reduced pressure to afford the title compound. ¹H NMR (400 MHz, CD₃OD): δ 6.96 (s; 1 H); 1.89 (d; J=9.54 Hz; 4 H).

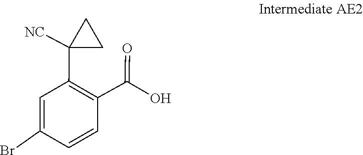 [SMILES: N#CC1(c2cc(Br)ccc2C(=O)O)CC1]

### 4-bromo-2-(1-cyanocyclopropyl)benzoic acid

### Step 1: methyl-4-bromo-2-(1-cyanocyclopropyl)benzoate

To a mixture of methyl-4-bromo-2(cyanomethyl)benzoate (4 g, 15.7 mmoL) in DMSO (23 mL) was added 2-(tert-butyl)-1,1,3,3-tetramethylgaunidine (5.4 g, 31.5 mmol) followed by 1-bromo-chloroethane (2.5 g, 17.32 mmol). The mixture was stirred at 23° C. for 18 h then diluted with EtOAc (20 mL), quenched with saturated ammonium chloride (20 mL), extracted with EtOAc (3×20 mL), washed with water (15 mL), washed with brine (15 mL), dried over Na₂SO₄, and concentrated under reduced pressure. The crude residue was purified by flash silica gel chromatography (0% to 30% EtOAc/Hex) to provide the title compound. MS (ESI) m/z: 280.1 [M+H]+.

### Step 2: 4-bromo-2-(1-cyanocyclopropyl)benzoic acid

To a mixture of methyl 4-bromo-2-(1-cyanocyclopropyl)benzoate (100 mg, 0.36 mmol) in DMSO (1.1 mL) was added sodium hydroxide (0.36 mL, 0.71 mmol, 2 M in water). The mixture was stirred at 23° C. for 10 min then quenched with aqueous HCl (1 mL, 1 M). The mixture was then diluted with water (10 mL) and extracted with EtOAc (3×10 mL). The combined organic layers were dried over Na₂SO₄, filtered, and concentrated under reduced pressure. ¹H NMR (499 MHz, CDCl₃) δ 7.49 (d, J=8.2 Hz, 1H), 7.38 (d, J=28.3 Hz, 1H), 7.23-7.18 (m, 1H), 2.20 (s, 1H), 1.34 (s, 2H), 1.05-0.66 (m, 3H). MS (ESI) m/z: 266.0 [M+H]+.

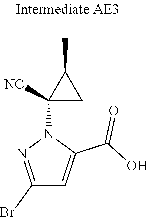

### 3-bromo-1-((1S,2S)-1-cyano-2-methylcyclopropyl)-1H-pyrazole-5-carboxylic acid

### Step 1: methyl 3-bromo-1-((1S,2S)-1-cyano-2-methylcyclopropyl)-1H-pyrazole-5-carboxylate

A solution of methyl 3-bromo-1-(cyanomethyl)-1H-pyrazole-5-carboxylate (66 g, 0.27 mol) and (R)-4-methyl-1,3,2-dioxathiolane 2,2-dioxide (94 g, 67 mL, 0.66 mol) in THF (1.3 L) cooled (0° C.) under an inert (Ar) atmosphere was treated with LiHMDS (1.0 M in THF) (0.13 kg, 0.80 L, 0.80 mol) (drop-wise addition over 1 h) and stirred at 0° C. for 1 h. The reaction mixture was quenched with a saturated aq NH₄Cl (1.0 L) at 0° C., then extracted with EtOAc (2×1.0 L). The combined organic layers were washed with water (1.0 L) then brine (1000 mL), dried over Na₂SO₄, filtered, and concentrated under reduced pressure. The crude residue was purified by flash silica column chromatography (2:1 EtOAc/heptane) to afford the product as a mixture of isomers. The mixture was purified by preparative SFC (i-Amylose-3 column, isopropylalcohol (0.1% NH₄OH) as a modifier). The fractions which contained the second eluting diastereomer were combined, concentrated in vacuo and co-evaporated with dichloromethane to afford the title compound. ¹H NMR (400 MHz, CDCl₃) δ 6.88 (s, 1H), 3.96 (s, 3H), 2.09 (dd, J=9.6, 6.5 Hz, 1H), 1.94 (ddd, J=9.6, 8.3, 6.3 Hz, 1H), 1.64-1.58 (m, 1H), 1.52 (d, J=6.3 Hz, 3H). MS (ESI) m/z: 283.9, 285.9 [M+H]+.

### Step 2: 3-bromo-1-((1S,2S)-1-cyano-2-methylcyclopropyl)-1H-pyrazole-5-carboxylic acid

A mixture of methyl 3-bromo-1-((1S,2S)-1-cyano-2-methylcyclopropyl)-1H-pyrazole-5-carboxylate (5 g, 17.6 mmol) in THF (50 mL) was added aq lithium hydroxide (886 mg, 21.1 mmol, 50 mL) at rt. Then, the reaction mixture was stirred at rt for 1 h. The mixture was poured into a solution of 0.5 M HCl (500 mL) and extracted with EtOAc (3×300 mL). The combined organic layers were washed with brine, dried over anhydrous Na₂SO₄, and concentrated under reduced pressure to afford the title compound. ¹H NMR (400 MHz, CDCl₃) δ 7.02 (s, 1H), 5.71 (s, 1H), 2.12 (dd, J=9.6, 6.5 Hz, 1H), 2.05-1.94 (m, 1H), 1.63 (dd, J=8.2, 6.6 Hz, 1H), 1.51 (d, J=6.2 Hz, 3H). MS (ESI) m/z: 270.0, 272.0 [M+H]⁺.

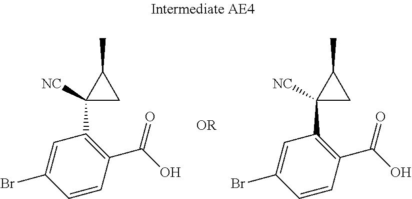 [SMILES: C[C@H]1C[C@]1(C#N)c1cc(Br)ccc1C(=O)O ; C[C@H]1C[C@@]1(C#N)c1cc(Br)ccc1C(=O)O]

### 4-bromo-2-((1R,2S)-1-cyano-2-methylcyclopropyl)benzoic acid or 4-bromo-2-((1S,2S)-1-cyano-2-methylcyclopropyl)benzoic acid

### Step 1: methyl 4-bromo-2-((1R,2S)-1-cyano-2-methylcyclopropyl)benzoate

At 0° C. under an inert (N₂) atmosphere, (R)-4-methyl-1,3,2-dioxathiolane 2,2-dioxide (19.1 g, 139 mmol) and methyl 4-bromo-2-(cyanomethyl)benzoate (17.6 g, 69.3 mmol) were mixed in THF (352 mL). Lithium bis(trimethylsilyl)amide (1.0 in THF) (29.0 g, 173 mL, 173 mmol) was added drop-wise over a period of 1 h and the mixture was subsequently stirred at 0° C. for 1 h. Then, the reaction mixture was poured into saturated aq NH₄Cl (400 mL), water (200 mL) and EtOAc (100 mL) were added. The layers were separated and the aq layer was extracted with EtOAc (2×200 mL). The combined organic layers were washed with brine, dried over Na₂SO₄, filtered, and concentrated in vacuo. The crude residue was purified using flash silica column chromatography (0 to 35% di-isopropyl ether/heptane) to obtain separated isomers as the titled compounds. Data for the first eluting isomer: ¹H NMR (400 MHz, CDCl₃) δ 7.83 (d, J=8.1 Hz, 1H), 7.59-7.51 (m, 2H), 3.98 (s, 3H), 1.54-1.41 (m, 5H), 1.41-1.32 (m, 1H). LCMS: MS (ESI) m/z: 294.0, 296.0 [M+H]+. Data for the second eluting isomer: ¹H NMR (400 MHz, CDCl₃) δ 7.90 (d, J=8.4 Hz, 1H), 7.62-7.52 (m, 2H), 3.98 (s, 3H), 2.00 (dp, J=9.0, 6.4 Hz, 1H), 1.86 (dd, J=9.0, 5.5 Hz, 1H), 1.19 (dd, J=7.0, 5.5 Hz, 1H), 0.80 (d, J=6.3 Hz, 3H). LCMS: MS (ESI) m/z: 294.0, 296.0 [M+H]+.

### Step 2: 4-bromo-2-((1R,2S)-1-cyano-2-methylcyclopropyl)benzoic acid or 4-bromo-2-((1S,2S)-1-cyano-2-methylcyclopropyl)benzoic acid

A solution of methyl 4-bromo-2-((1R,2S)-1-cyano-2-methylcyclopropyl)benzoate or methyl 4-bromo-2-((1S,2S)-1-cyano-2-methylcyclopropyl)benzoate (6.49 g, 22.1 mmol, first eluting isomer from previous step) in THF (130 mL) at rt was added lithium hydroxide monohydrate (1.11 g, 26.5 mmol) in water (130 mL). The resulting mixture was stirred at rt for 3.5 h. Additional lithium hydroxide monohydrate (185 mg, 4.41 mmol) was added and stirring was continued for 18 h. A third portion of lithium hydroxide monohydrate (185 mg, 4.41 mmol) was added and the mixture was stirred for 2 h. Then, the mixture was poured into aq 0.5 M HCl (250 mL) and extracted with EtOAc (3×100 mL). The combined organic layers were dried over Na₂SO₄, filtered, and concentrated in vacuo to obtain the title compound. ¹H NMR (400 MHz, DMSOd₆) δ 13.46 (s, 1H), 7.83-7.73 (m, 2H), 7.70 (dd, J=8.3, 2.0 Hz, 1H), 1.66 (dd, J=8.6, 5.4 Hz, 1H), 1.59-1.46 (m, 1H), 1.43-1.27 (m, 4H). MS (ESI) m/z: 280.0, 282.0 [M+H]⁺.

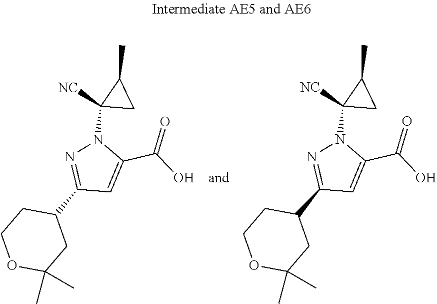

### 1-((1S,2S)-1-cyano-2-methylcyclopropyl)-3-((S)-2,2-dimethyltetrahydro-2H-pyran-4-yl)-1H-pyrazole-5-carboxylic acid and 1-((1S,2S)-1-cyano-2-methylcyclopropyl)-3-((S)-2,2-dimethyltetrahydro-2H-pyran-4-yl)-1H-pyrazole-5-carboxylic acid

### Step 1: methyl 1-((1S,2S)-1-cyano-2-methylcyclopropyl)-3-((S)-2,2-dimethyltetrahydro-2H-pyran-4-yl)-1H-pyrazole-5-carboxylate AND methyl 1-((1S,2S)-1-cyano-2-methylcyclopropyl)-3-((S)-2,2-dimethyltetrahydro-2H-pyran-4-yl)-1H-pyrazole-5-carboxylate

To a vial was added methyl 3-bromo-1-((1S,2S)-1-cyano-2-methylcyclopropyl)-1H-pyrazole-5-carboxylate (200 mg, 704 mol), 2,2-dimethyltetrahydro-2H-pyran-4-carboxylic acid (167 mg, 1.06 mmol), isoindoline-1,3-dione (104 mg, 704 mol), tetramethly-phenanthroline nickel di-bromide (66.2 mg, 141 mol), (Ir[dF(CF₃)ppy]₂(dtbpy))PF₆ (15.8 mg, 14.1 mol), and 2-(tert-butyl)-1,1,3,3-tetramethylguanidine (301 mg, 0.35 mL, 1.76 mmol), and DMSO (6 mL). The vial was sealed inside the glove box and irradiated under 450 nm LED modules at 100% light intensity for 24 hours. The mixture was quenched with sat. NH₄Cl and extracted with EtOAc. The combined organic layers were washed with brine, dried over MgSO₄, filtered, and concentrated under reduced pressure. The crude material was purified by column chromatography on silica gel (0-40% EtOAc/hexanes) to afford the title compound as a racemate. The racemic material was separated using SFC (Whelk-O® 1 (S,S), 5% IPA in MeOH) to afford the two isomers. Isomer 1: MS (ESI) m/z: 317.0. Isomer 2: MS (ESI) m/z: 317.0.

### Step 2: 1-((1S,2S)-1-cyano-2-methylcyclopropyl)-3-((S)-2,2-dimethyltetrahydro-2H-pyran-4-yl)-1H-pyrazole-5-carboxylic acid OR 1-((1S,2S)-1-cyano-2-methylcyclopropyl)-3-((S)-2,2-dimethyltetrahydro-2H-pyran-4-yl)-1H-pyrazole-5-carboxylic acid

To a solution of methyl 1-((1S,2S)-1-cyano-2-methylcyclopropyl)-3-(2,2-dimethyltetrahydro-2H-pyran-4-yl)-1H-pyrazole-5-carboxylate (56 mg, 0.18 mmol) and methyl 1-((1S,2S)-1-cyano-2-methylcyclopropyl)-3-(2,2-dimethyltetrahydro-2H-pyran-4-yl)-1H-pyrazole-5-carboxylate (54 mg, 0.17 mmol) in DMSO (1 ml) was added sodium hydroxide (0.35 mL, 1 M, 0.35 mmol) and the resultant reaction was stirred at room temperature for 1 hour. The reaction mixture was diluted by water (1 mL), adjusted to pH=3 with 1 M HCl (˜0.5 mL), and extracted with EtOAc (3×50 mL). The combined organic layers were washed with brine, dried over MgSO₄, filtered, and concentrated under reduced pressure to afford the title compound. MS (ESI) m/z: 304.0.

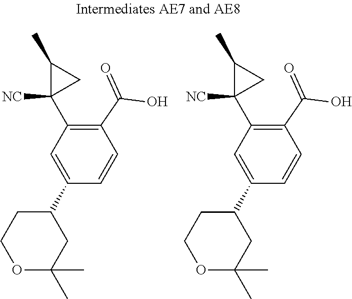

### 2-((1R,2S)-1-cyano-2-methylcyclopropyl)-4-((S)-2,2-dimethyltetrahydro-2H-pyran-4-yl)benzoic acid AND 2-((1R,2S)-1-cyano-2-methylcyclopropyl)-4-((S)-2,2-dimethyltetrahydro-2H-pyran-4-yl)benzoic acid

### Step 1: methyl 2-((1R,2S)-1-cyano-2-methylcyclopropyl)-4-((S)-2,2-dimethyltetrahydro-2H-pyran-4-yl)benzoate and methyl 2-((1R,2S)-1-cyano-2-methylcyclopropyl)-4-((S)-2,2-dimethyltetrahydro-2H-pyran-4-yl)benzoate

This step was performed in a similar fashion as described in Step 1 towards Intermediates AE5 and AE6. SFC conditions used to separate isomers: ColumnTek Enantiocel® A6-5, 20% methanol (0.1% DEA). Isomer 1: MS (ESI) m/z: 328.2. Isomer 2: MS (ESI) m/z: 328.2.

### Step 2: 2-((1R,2S)-1-cyano-2-methylcyclopropyl)-4-((S)-2,2-dimethyltetrahydro-2H-pyran-4-yl)benzoic acid and 2-((1R,2S)-1-cyano-2-methylcyclopropyl)-4-((S)-2,2-dimethyltetrahydro-2H-pyran-4-yl)benzoic acid

This step was performed in a similar fashion as described in Step 2 towards Intermediates AE5 and AE6. MS (ESI) m/z: 314.2.

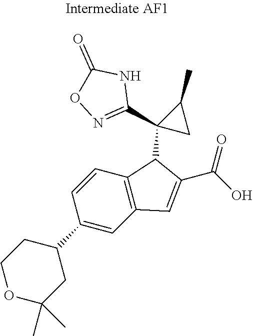

### 5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carboxylic acid

### Step 1: ethyl 5-(2,2-dimethyl-3,6-dihydro-2H-pyran-4-yl)-1H-indole-2-carboxylate and ethyl 5-(6,6-dimethyl-3,6-dihydro-2H-pyran-4-yl)-1H-indole-2-carboxylate

To a solution of a 1:1 mixture of 2-(2,2-dimethyl-3,6-dihydro-2H-pyran-4-yl)-4,4,5,5-tetramethyl-1,3,2-dioxaborolane and 2-(6,6-dimethyl-3,6-dihydro-2H-pyran-4-yl)-4,4,5,5-tetramethyl-1,3,2-dioxaborolane (101 g, 424 mmol) in dioxane (1.30 L) at 20° C. was added H₂O (130 mL), ethyl 5-bromo-1H-indole-2-carboxylate (114 gm 424 mmol), K₂CO₃ (117 g, 848 mmol) and Pd(dppf)Cl₂ (9.31 g, 12.7 mmol). The atmosphere of the reaction mixture was replaced with N₂ (via 3 purge-pump cycles) and then stirred for 12 h at 90° C. Upon reaction completion, the mixture was cooled to 20° C., quenched with H₂O (0.50 L) and EtOAc (0.5 L), filtered and then extracted with EtOAc (3×0.5 L). The combined organic layers were dried over anhydrous Na₂SO₄, filtered, and then concentrated under reduced pressure. This protocol was carried out twice in parallel and the combined crude residue was purified sequentially by silica gel chromatography (petroleum ether/EtOAc: 1/0 to 85/15) and then reverse-phase HPLC (MeCN/H₂O+0.1% TFA 40% to 75% gradient) to provide the title compounds as a mixture. TLC Rf 0.4 (petroleum ether/EtOAc: 3/1). MS: 300.1.

### Step 2: ethyl (S)-5-(2,2-dimethyltetrahydro-2H-pyran-4-yl)-1H-indole-2-carboxylate

To a solution Pd/C (18.8 g, 17.7 mmol, 10% by wt) in MeOH (470 mL) and EtOAc (470 mL) at 20° C. under an atmosphere of Ar was added the mixture of ethyl 5-(2,2-dimethyl-3,6-dihydro-2H-pyran-4-yl)-1H-indole-2-carboxylate and ethyl 5-(6,6-dimethyl-3,6-dihydro-2H-pyran-4-yl)-1H-indole-2-carboxylate (94.0 g, 289 mmol). The atmosphere of the reaction mixture was replaced with H₂ (via 3 purge-pump cycles) and then stirred under H₂ (15 Psi) at 20° C. for 4 h. Upon reaction completion, the mixture was filtered and concentrated under reduced pressure. The enantiomers of the resultant product were separated by SFC (column: DAICEL CHIRALPAK i.e., i.e., (50*250 mm, 10 m); mobile phase: [CO₂—4:1 EtOH/MeCN]; B %: 55%, isocratic elution mode) to obtain the title compound as the slower eluting isomer. ¹H NMR: (400 MHz, DMSO-d6) δ 11.76 (br s, 1H), 7.47 (s, 1H), 7.38 (br d, J=8.5 Hz, 1H), 7.17 (br d, J=8.6 Hz, 1H), 7.08 (s, 1H), 4.33 (q, J=7.0 Hz, 2H), 3.69 (br d, J=8.3 Hz, 2H), 2.98 (br t, J=12.3 Hz, 1H), 1.71-1.63 (m, 2H), 1.62-1.43 (m, 2H), 1.33 (t, J=7.1 Hz, 3H), 1.26 (s, 3H), 1.17 (s, 3H).

### Step 3: (S)-5-(2,2-dimethyltetrahydro-2H-pyran-4-yl)-1H-indole-2-carboxylic acid

To a solution of ethyl (S)-5-(2,2-dimethyltetrahydro-2H-pyran-4-yl)-1H-indole-2-carboxylate (48.0 g, 127 mmol) in MeOH (720 mL) at 20° C. was added NaOH (2M aqueous, 140 mL) to the mixture. The reaction mixture was stirred at 65° C. for 1 h. Upon reaction completion, the mixture was cooled to 15° C. and HCl (5N aqueous, 58.6 mL) was added slowly (by drop-wise addition). Water (360 mL) was added slowly and then the resultant solid precipitate was collected by filtration. After washing with water (240 mL), the solid was dried under reduced pressure to provide the title compound. ¹H NMR: (400 MHz, DMSO-d6) δ 12.88 (br s, 1H), 11.63 (br s, 1H), 7.46 (s, 1H), 7.35 (d, J=8.6 Hz, 1H), 7.15 (br d, J=8.6 Hz, 1H), 7.02 (s, 1H), 3.70 (br d, J=7.6 Hz, 2H), 3.05-2.88 (m, 1H), 1.68 (br d, J=11.9 Hz, 2H), 1.63-1.43 (m, 2H), 1.26 (s, 3H), 1.17 (s, 3H).

### Step 4: (S)-5-(2,2-dimethyltetrahydro-2H-pyran-4-yl)-N-methyl-N-phenyl-1H-indole-2-carboxamide

To a solution of (S)-5-(2,2-dimethyltetrahydro-2H-pyran-4-yl)-1H-indole-2-carboxylic acid (33.0 g, 121 mmol) in DMA (330 mL) under an atmosphere of N₂ and cooled to 0-10° C. was added SOCl₂ (17.2 g, 145 mmol) slowly by drop-wise addition. The mixture was stirred for 1 h at 0-10° C., then N-methylaniline (15.5 g, 145 mmol) and Et₃N (29.3 g, 290 mmol) were added. The resulting mixture was stirred at 20° C. for 1 h. Upon reaction completion, the mixture was quenched by the slow addition of water (165 mL) and then the resultant solid precipitate was collected by filtration. After washing with water (330 mL), the solid was dried under reduced pressure to provide the title compound.

### Step 5: (S)-1-(cyanomethyl)-5-(2,2-dimethyltetrahydro-2H-pyran-4-yl)-N-methyl-N-phenyl-1H-indole-2-carboxamide

To a solution of (S)-5-(2,2-dimethyltetrahydro-2H-pyran-4-yl)-N-methyl-N-phenyl-1H-indole-2-carboxamide (42.0 g, 114 mmol) in 1,3-Dimethyl-2-imidazolidinone was added KOH (8M aqueous, 42.8 mL) and H₂O (42.0 mL). The reaction mixture was cooled to 10° C. and 2-chloroacetonitrile (12.9 g, 171 mmol) was added. The reaction mixture was stirred at 10° C. for 2.5 h. Upon reaction completion, the mixture was quenched by the addition of HCl (5N aqueous, 79.9 mL) and H₂O (42.0 mL) at 10° C. The resulting mixture was extracted with cyclopentyl methyl ether (2×420 mL). The combined organic layers were washed with brine and then concentrated under reduced pressure to provide the title compound. MS: 402.3.

### Step 6: 1-((1S,2S)-1-cyano-2-methylcyclopropyl)-5-((S)-2,2-dimethyltetrahydro-2H-pyran-4-yl)-N-methyl-N-phenyl-1H-indole-2-carboxamide

To a solution of (S)-1-(cyanomethyl)-5-(2,2-dimethyltetrahydro-2H-pyran-4-yl)-N-methyl-N-phenyl-1H-indole-2-carboxamide (54.2 g, 112 mmol) in N,N-dimethylpropyleneurea (540 mL) was added (4R)-4-methyl-1,3,2-dioxathiolane 2,2-dioxide (46.4 g, 336 mmol) to the mixture. The mixture was cooled to 0° C. under an atmosphere of N₂ then KHMDS (1 M, 448 mL) was added slowly by drop-wise addition. The resulting mixture was stirred at 0° C. for 2.5 h. Upon reaction completion, the mixture was quenched by the addition of formic acid (50.7 mL) slowly at 0° C. then water (270 mL). The mixture was extracted with petroleum ether/EtOAc (1:3 2×540 mL). The combined organic layers were washed with water (3×270 mL), 10% aq. NaHCO₃ (2×540 mL), brine (270 mL) then dried over Na₂SO₄, filtered, and concentrated under reduced pressure. The crude residue was purified by silica gel chromatography (petroleum ether/EtOAc: 19/1 to 3/1) to provide the title compound. ¹H NMR: (400 MHz, DMSO-d6) δ 7.50-7.30 (m, 5H), 7.30-7.21 (m, 3H), 5.94 (br s, 1H), 3.66 (br d, J=7.3 Hz, 2H), 3.43 (s, 3H), 3.02-2.89 (m, 1H), 1.98-1.89 (m, 1H), 1.89-1.69 (m, 2H), 1.66-1.37 (m, 7H), 1.22 (s, 3H), 1.14 (s, 3H). MS: 442.4.

### Step 7: 5-((S)-2,2-dimethyltetrahydro-2H-pyran-4-yl)-N-methyl-1-((1S,2S)-2-methyl-1-(5-oxo-4,5-dihydro-1,2,4-oxadiazol-3-yl)cyclopropyl)-N-phenyl-1H-indole-2-carboxamide

To a solution of 1-((1S,2S)-1-cyano-2-methylcyclopropyl)-5-((S)-2,2-dimethyltetrahydro-2H-pyran-4-yl)-N-methyl-N-phenyl-1H-indole-2-carboxamide (20.7 g, 46.6 mmol) in DMSO (248 mL) was hydroxylamine (30.8 g, 466 mmol). The reaction mixture was stirred at 25° C. for 17 h under an atmosphere of N₂ then quenched by the addition of water (250 mL) and EtOAc (125 mL). The resultant solid precipitate was collected by filtration. After washing with water (250 mL), the solid was dried under reduced pressure. The filtrate was further extracted with EtOAc (125 mL). The organic phase was washed with brine (125 mL), dried over Na₂SO₄, filtered, and concentrated under reduced pressure. The combined crude residues were dissolved in DMSO (166 mL), and CDI (15.1 g, 93.1 mmol) and then DBU (17.7 g, 116 mmol) was added. The mixture was stirred at 25° C. for 0.5 h under an atmosphere of N₂. Upon reaction completion, the mixture was quenched by the addition of formic acid (to pH<3) at 15° C. then water (100 mL) was added slowly by drop-wise addition. The resultant solid precipitate was collected by filtration. After washing with water (200 mL), the solid was dried under reduced pressure. The crude residue was purified sequentially by trituration (petroleum ether/EtOAc=5/1, 200 mL) then silica gel chromatography (petroleum ether/EtOAc: 10/1 to 1/2) to provide the title compound. ¹H NMR: (400 MHz, DMSO-d6) δ 12.36-11.57 (m, 1H), 7.45-7.15 (m, 8H), 6.11-5.82 (m, 1H), 3.66 (br d, J=7.8 Hz, 2H), 3.52-3.35 (m, 3H), 2.93 (br t, J=12.1 Hz, 1H), 2.11-1.88 (m, 1H), 1.86-1.54 (m, 4H), 1.54-1.24 (m, 5H), 1.22 (s, 3H), 1.14 (s, 3H). MS: 501.3.

### Step 8: 5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carboxylic acid

To a solution of 5-((S)-2,2-dimethyltetrahydro-2H-pyran-4-yl)-N-methyl-1-((1S,2S)-2-methyl-1-(5-oxo-4,5-dihydro-1,2,4-oxadiazol-3-yl)cyclopropyl)-N-phenyl-1H-indole-2-carboxamide (15.0 g, 29.2 mmol), in 2-methoxyethanol (75 mL) was added KOH (24.6 g, 439 mmol). The reaction mixture was stirred at 100° C. for 4 h under an atmosphere of N₂. Upon reaction completion, the mixture was cooled to 0-5° C. and quenched by the addition of HCl (4N aqueous, 97.5 mL). The suspension was stirred for 0.5 h at 20° C. then the resultant solid precipitate was collected by filtration. After washing with water (150 mL), the solid was dried under reduced pressure. The crude residue was suspended in with water (75 mL) and extracted with THF/EtOAc (1/1 2×75 mL). The combined organic layers were washed with brine (75 mL) then dried over Na₂SO₄, filtered, and concentrated under reduced pressure. The crude residue was purified by trituration (petroleum ether/EtOAc=1/1, 150 mL). The solid was redissolved with MeCN (75 mL) and water (75 mL), filtered and dried by lyophilization to provide the title compound. ¹H NMR: (400 MHz, DMSO-d6) δ 12.88 (br s, 1H), 12.47-11.74 (m, 1H), 7.53 (d, J=5.0 Hz, 1H), 7.39-7.33 (m, 1H), 7.32-7.24 (m, 1H), 7.19 (d, J=4.3 Hz, 1H), 3.70 (br dd, J=2.0, 4.4 Hz, 2H), 3.09-2.95 (m, 1H), 2.04-1.86 (m, 1H), 1.85-1.73 (m, 1H), 1.72-1.45 (m, 5H), 1.43-1.22 (m, 6H), 1.18 (d, J=5.1 Hz, 3H). MS: 410.2 (negative mode). The following intermediates in table AF were prepared using the procedures outlined in the synthesis of intermediate AF1.

TABLE AF

| Intermediate | Structure | Name | NMR and MS (M + 1) |
|---|---|---|---|
| AF2 | 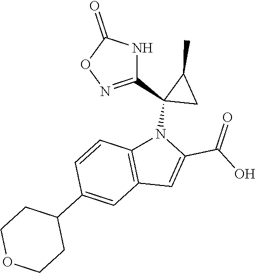 [SMILES: C[C@H]1C[C@]1(c1noc(=O)[nH]1)n1c(C(=O)O)cc2cc(C3CCOCC3)ccc21] | 1-((1S,2S)-2- methyl-1-(5-oxo- 4,5-dihydro-1,2,4- oxadiazol-3- yl)cyclopropyl)-5- (tetrahydro-2H- pyran-4-yl)-1H- indole-2-carboxylic acid | ¹H NMR: (400 MHz, CDCl₃) δ 12.9 (br d, J = 2.6 H, 1H), 12.3 (br s, 1H), 11.9-12.1 (m, 1H), 7.54 (d, J = 4.5 Hz, 1H), 7.34- 7.42 (m, 1H), 7.26-7.33 (m, 1H), 7.20 (d, J = 4.0 Hz, 1H), 3.92- 4.00 (m, 2H), 3.31-3.54 (m, 3H), 2.79-2.90 (m, 1H), 1.89-2.07 (m, 1H), 1.68-1.86 (m, 5H), 1.53- 1.67 (m, 1H), 1.40 (d, J = 6.4 Hz, 1.3H), 1.28 (d, J = 6.4 Hz, 1.7H). MS: 384.1 |
| AF3 | 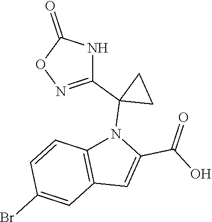 [SMILES: O=C(O)c1cc2cc(Br)ccc2n1C1(c2noc(=O)[nH]2)CC1] | 5-bromo-1-(1-(5- oxo-4,5-dihydro- 1,2,4-oxadiazol-3- yl)cyclopropyl)- 1H-indole-2- carboxylic acid | ¹H NMR (500 MHz, DMSO) δ 7.92 (d, J = 1.7 Hz, 1H), 7.57 (d, J = 8.9 Hz, 1H), 7.46 (dd, J = 8.9, 1.8 Hz, 1H), 7.20 (s, 1H), 2.01- 1.90 (m, 2H), 1.81 (dd, J = 11.4, 4.0 Hz, 1H), 1.67 (q, J = 10.5 Hz, 2H). |

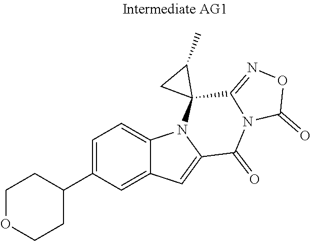 [SMILES: C[C@H]1C[C@@]12c1noc(=O)n1C(=O)c1cc3cc(C4CCOCC4)ccc3n12]

### (1S,2S)-2-methyl-8′-(tetrahydro-2H-pyran-4-yl)-3′H,5′H-spiro[cyclopropane-1,12′-[1,2,4]oxadiazolo[4′,3′:4,5]pyrazino[1,2-a]indole]-3′,5′-dione

A mixture of 1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carboxylic acid (1.21 g, 3.16 mmol) and HATU (1.44 g, 3.79 mmol) in DMF (10.5 mL) was cooled to 0° C. then treated with DIPEA (612 mg, 4.73 mmol) via slow (drop-wise) addition. The reaction mixture was allowed to stir while warming to rt. After 15 min, the mixture was treated with H2O (˜40 mL) and the resulting solid precipitate was collected by filtration with additional washing with DCM then dried under reduced pressure to afford the title compound. ¹H NMR (500 MHz, DMSO-d6) δ 7.64 (s, 1H), 7.62 (s, 1H), 7.56 (d, J=9.0 Hz, 1H), 7.33 (dd, J=9.0, 1.6 Hz, 1H), 3.96 (dd, J=10.8, 3.2 Hz, 2H), 3.45 (td, J=11.3, 2.5 Hz, 2H), 3.16 (dd, J=9.9, 7.6 Hz, 1H), 2.86 (tt, J=11.1, 4.8 Hz, 1H), 2.68 (dq, J=9.7, 4.8 Hz, 1H), 1.79-1.63 (m, 4H), 1.61-1.55 (m, 1H), 1.30 (d, J=6.1 Hz, 3H). MS (ESI) m/z: 388.4 [M+Na]+.

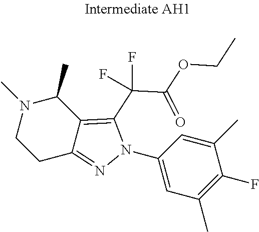 [SMILES: CCOC(=O)C(F)(F)c1c2c(nn1-c1cc(C)c(F)c(C)c1)CCN(C)[C@H]2C]

### tert-butyl (S)-3-(2-ethoxy-1,1-difluoro-2-oxoethyl)-2-(4-fluoro-3,5-dimethylphenyl)-4-methyl-2,4,6,7-tetrahydro-5H-pyrazolo[4,3-c]pyridine-5-carboxylate

A vial containing tert-butyl (S)-3-bromo-2-(4-fluoro-3,5-dimethylphenyl)-4-methyl-2,4,6,7-tetrahydro-5H-pyrazolo[4,3-c]pyridine-5-carboxylate (100 mg, 0.228 mmol) and copper powder (29.0 mg, 0.456 mmol) was placed under argon. To the vial was then added DMSO (0.57 mL) and ethyl-2-bromo-2,2-difluoroacetate (69.5 mg, 44 μL, 0.342 mmol). The reaction mixture was stirred under argon at 80° C. overnight. At this point, additional copper (40.0 mg, 0.629 mmol) and ethyl-2-bromo-2,2-difluoroacetate (94.7 mg, 60 μL, 0.342 mmol) were added. The mixture was stirred under argon at 100° C. for 1.5 h. The mixture was then cooled down, diluted with DCM, filtered, and concentrated. The residue was purified via silica gel column chromatography (0-50% EtOAc/hexanes) to afford the title compound as a solid. MS (ESI): m/z 482.6 [M+H]+.

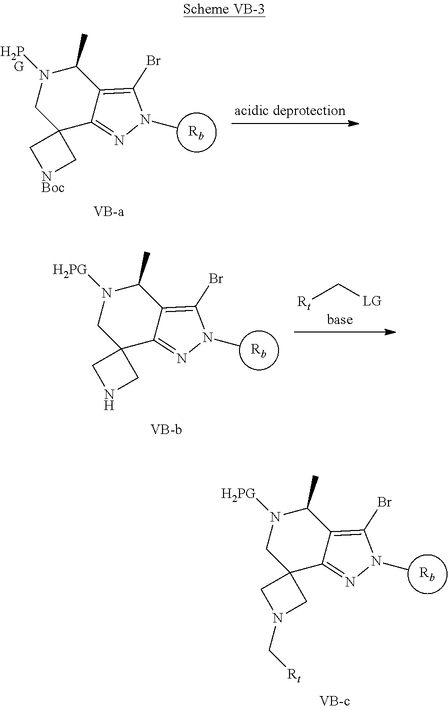

Intermediate VB-c is prepared through an acidic deprotection of compounds of the form VB-a where R_{b} is aryl, heteroaryl, alkyl, cycloalkyl, or heterocycle. Alkylation using basic conditions provides compounds of the form VB-c where Rₜ is aryl, heteroaryl, linear alkyl, cycloalkyl or saturated heterocycle.

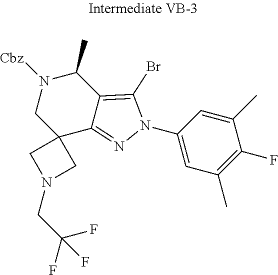 [SMILES: Cc1cc(-n2nc3c(c2Br)[C@H](C)N(C(=O)OCc2ccccc2)CC32CN(CC(F)(F)F)C2)cc(C)c1F]

### benzyl (S)-3′-bromo-2′-(4-fluoro-3,5-dimethylphenyl)-4′-methyl-1-(2,2,2-trifluoroethyl)-2′,4′-dihydrospiro[azetidine-3,7′-pyrazolo[4,3-c]pyridine]-5′(6′H)-carboxylate

### Step 1: benzyl (S)-3′-bromo-2′-(4-fluoro-3,5-dimethylphenyl)-4′-methyl-2′,4′-dihydrospiro[azetidine-3,7′-pyrazolo[4,3-c]pyridine]-5′(6′H)-carboxylate

A mixture of 5′-benzyl 1-(tert-butyl) (S)-3′-bromo-2′-(4-fluoro-3,5-dimethylphenyl)-4′-methyl-2′,4′-dihydrospiro[azetidine-3,7′-pyrazolo[4,3-c]pyridine]-1,5′(6′H)-dicarboxylate (600 mg, 0.98 mmol) and HCl in dioxane (2M, 6 mL, 12 mmol) was stirred at 15° C. for 16 h. The mixture was concentrated under reduced pressure to provide the title compound, which was used in the next step without further purification. MS (ESI) m/z: 512.8, 514.8 [M+H]⁺.

### Step 2: benzyl (S)-3′-bromo-2′-(4-fluoro-3,5-dimethylphenyl)-4′-methyl-1-(2,2,2-trifluoroethyl)-2′,4′-dihydrospiro[azetidine-3,7′-pyrazolo[4,3-c]pyridine]-5′(6′H)-carboxylate

To a mixture of benzyl (S)-3′-bromo-2′-(4-fluoro-3,5-dimethylphenyl)-4′-methyl-2′,4′-dihydrospiro[azetidine-3,7′-pyrazolo[4,3-c]pyridine]-5′(6′H)-carboxylate (500 mg, 0.97 mmol) and TEA (0.49 g, 0.68 mL, 4.9 mmol) in DMF (5 mL) was added 2,2,2-trifluoroethyl trifluoromethanesulfonate (0.34 g, 1.5 mmol), and the reaction mixture was stirred for 1.5 h at 15° C. The reaction mixture was then diluted with water (5 mL) and extracted with EtOAc (3×5 mL). The combined organic layers were dried over Na₂SO₄, filtered, and concentrated under reduced pressure. The residue was purified via silica gel column chromatography (0-33% EtOAc/hexanes) to provide the title compound. MS (ESI) m/z: 596.4 [M+H]⁺.

The following intermediates in table DW were prepared using the procedures outlined in Scheme VB-3 (Intermediate VB-3).

TABLE DW

| Intermediate | Structure | Name | MS (M + H) |
|---|---|---|---|
| DW-1 | 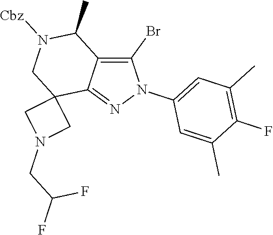 [SMILES: Cc1cc(-n2nc3c(c2Br)[C@H](C)N(C(=O)OCc2ccccc2)CC32CN(CC(F)F)C2)cc(C)c1F] | benzyl (S)-3′-bromo-1- (2,2-difluoroethyl)-2-(4-fluoro- 3,5-dimethylphenyl)-4′-methyl- 2′,4′-dihydrospiro[azetidine-3,7′- pyrazolo[4,3-c]pyridine]-5′(6′H)- carboxylate | 576.9 |

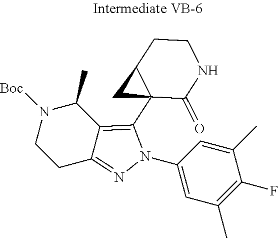 [SMILES: Cc1cc(-n2nc3c(c2[C@]24C[C@H]2CCNC4=O)[C@H](C)N(C(=O)OC(C)(C)C)CC3)cc(C)c1F]

### tert-butyl (S)-2-(4-fluoro-3,5-dimethylphenyl)-4-methyl-3-((1S,6S)-2-oxo-3-azabicyclo[4.1.0]heptan-1-yl)-2,4,6,7-tetrahydro-5H-pyrazolo[4,3-c]pyridine-5-carboxylate

### Step 1: (S)-(5-(tert-butoxycarbonyl)-2-(4-fluoro-3,5-dimethylphenyl)-4-methyl-4,5,6,7-tetrahydro-2H-pyrazolo[4,3-c]pyridin-3-yl)boronic acid

A stirred mixture of tert-butyl (S)-3-bromo-2-(4-fluoro-3,5-dimethylphenyl)-4-methyl-2,4,6,7-tetrahydro-5H-pyrazolo[4,3-c]pyridine-5-carboxylate (10.0 g, 22.8 mmol) in anhydrous THF (46 mL) cooled to −78° C. was treated with n-butyllithium (2.5M in hexanes, 11.0 mL, 27.4 mmol) dropwise. The reaction mixture was stirred at −78° C. for 20 min. To the mixture was then added trimethyl borate (5.1 mL, 4.7 g, 46 mmol) dropwise at −78° C. The reaction mixture was then allowed to return to rt and stirred for 36 h. The mixture was quenched with 1M aqueous HCl, stirred for 3 min, then extracted with EtOAc (3×). The combined organic layers were washed with brine, dried over MgSO₄, and concentrated under reduced pressure. The residue was dissolved in DCM (˜30 mL) and then treated with hexanes (˜300 mL). The resultant precipitate was collected via vacuum filtration, rinsed with hexanes, and air-dried to afford the title compound, which was used in the next step without further purification. MS (ESI) m/z: 404.2 [M+H]⁺.

### Step 2: tert-butyl (S)-2-(4-fluoro-3,5-dimethylphenyl)-4-methyl-3-(2-oxo-1,2,5,6-tetrahydropyridin-3-yl)-2,4,6,7-tetrahydro-5H-pyrazolo[4,3-c]pyridine-5-carboxylate

A stirred mixture of (S)-(5-(tert-butoxycarbonyl)-2-(4-fluoro-3,5-dimethylphenyl)-4-methyl-4,5,6,7-tetrahydro-2H-pyrazolo[4,3-c]pyridin-3-yl)boronic acid (1.5 g, 3.8 mmol), [1,1′-bis(diphenylphosphino)ferrocene]dichloropalladium(II) complex with dichloromethane (0.62 g, 0.76 mmol) and 3-bromo-5,6-dihydropyridin-2(1H)-one (0.44 g, 2.5 mmol) in dioxane (36 mL) was sparged with argon for 5 min. To the mixture was then added an aqueous K₂CO₃ solution (1M, 6.3 mL, 6.3 mmol) that was pre-sparged with argon for 5 min. The reaction mixture was then stirred under argon at 80° C. for 45 min. The mixture was cooled to rt, concentrated, redissolved in DCM, and filtered. The filtrate was concentrated. The residue was purified via silica gel column chromatography (0-100% (3:1 EtOAc/EtOH) in hexanes) to provide the title compound. MS (ESI) m/z: 455.2 [M+H]⁺.

### Step 3: tert-butyl (S)-2-(4-fluoro-3,5-dimethylphenyl)-4-methyl-3-((1S,6S)-2-oxo-3-azabicyclo[4.1.0]heptan-1-yl)-2,4,6,7-tetrahydro-5H-pyrazolo[4,3-c]pyridine-5-carboxylate

Trimethyl(oxo)sulfonium iodide (0.70 g, 3.2 mmol) was added to a stirred suspension of sodium hydride (60% wt, 0.14 g, 3.5 mmol) in DMSO (3.5 mL). The mixture was stirred at 40° C. for 25 min. The mixture was cooled to rt and added to a solution of tert-butyl (S)-2-(4-fluoro-3,5-dimethylphenyl)-4-methyl-3-(2-oxo-1,2,5,6-tetrahydropyridin-3-yl)-2,4,6,7-tetrahydro-5H-pyrazolo[4,3-c]pyridine-5-carboxylate (0.48 g, 1.1 mmol) in DMSO (3.5 mL). The reaction mixture was stirred at rt, with occasional swirling, for 50 min. The mixture was quenched with formic acid (0.16 mL, 4.2 mmol), syringe filtered, and purified via reverse-phase prep-HPLC (1 peak 0-100% MeCN/water with 0.1% formic acid modifier) to afford the title compound (minor diastereomer, early-eluting). ¹H NMR (500 MHz, CD₃CN) δ 7.02 (d, J=6.0 Hz, 2H), 6.03 (s, 1H), 5.18 (br s, 1H), 4.23 (br s, 1H), 3.16 (br s, 1H), 3.07-2.93 (m, 2H), 2.69-2.54 (m, 2H), 2.27 (d, J=2.0 Hz, 6H), 1.81-1.70 (m, 2H), 1.45 (s, 9H), 1.40 (d, J=6.6 Hz, 3H), 1.37-1.28 (m, 2H), 1.25-1.16 (m, 1H). MS (ESI) m/z: 469.2 [M+H]⁺.

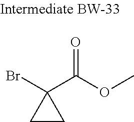

### (1-(methoxycarbonyl)cyclopropyl)zinc(II) bromide

To a suspension of zinc dust (3.06 g, 46.8 mmol, 2 equiv) in THF (60 mL) was added TMS-Cl (0.30 mL, 2.3 mmol, 0.1 equiv) and 1,2-dibromoethane (0.40 mL, 4.7 mmol, 0.2 equiv) under argon atmosphere. The mixture was stirred for 30 min before methyl 1-bromocyclopropane-1-carboxylater (3.00 mL, 23.4 mmol, 1 equiv) was added and the resulting mixture was stirred for 2 h at 60° C. The cloudy gray suspension was titrated with I₂ and the concentration determined to be 0.35 M.

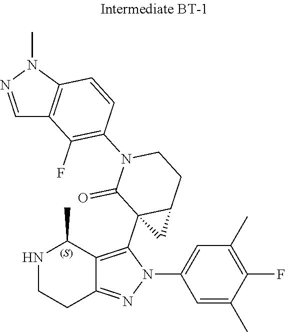

### (1S,6S)-3-(4-fluoro-1-methyl-1H-indazol-5-yl)-1-((S)-2-(4-fluoro-3,5-dimethylphenyl)-4-methyl-4,5,6,7-tetrahydro-2H-pyrazolo[4,3-c]pyridin-3-yl)-3-azabicyclo[4.1.0]heptan-2-one

### Step 1: 1-(4-fluoro-1-methyl-1H-indazol-5-yl)piperidin-2-one

A solution of 5-bromo-4-fluoro-1-methyl-1H-indazole (7.5 g, 32.7 mmol), piperidin-2-one (81 g, 0.82 mol), CuI (6.2 g, 33 mmol), K₂CO₃ (14 g, 98 mmol) and N¹, N²-dimethylethane-1,2-diamine (3.2 g, 36 mmol) in DMF (20 mL) was stirred for 3 hours at 135° C. After cooling to rt, the reaction mixture was filtered through Celite® pad and washed with EtOAc (3×70 mL). The filtrate was concentrated under reduced pressure. EtOAc (100 mL) and water (80 mL) were then added, and the layers were separated. The organic layer was washed with brine (30 mL×2), dried over Na₂SO₄, filtered and concentrated under reduced pressure. The material was then purified by reverse phase chromatography (Cis, 120 g, flow rate of 70 mL/min, run time of 22 CV) using a gradient of 5-100% acetonitrile and water (contains 0.1% formic acid) to provide the title compound. MS (ESI) m/z: 248.0 [M+H]⁺.

### Step 2: 3,3-dichloro-1-(4-fluoro-1-methyl-1H-indazol-5-yl)piperidin-2-one

PCl₅ (7.8 g, 38 mmol) was added to a solution of 1-(4-fluoro-1-methyl-1H-indazol-5-yl)piperidin-2-one (3.1 g, 13 mmol) in chloroform (60 mL) at 65° C. The reaction mixture was stirred at the same temperature for 3 hours. The cooled reaction mixture was then slowly poured onto ice water (60 mL) and vigorously stirred while keeping the internal temperature below 20° C. After stirring for an additional 10 min, the mixture was extracted with DCM (60 mL), and the extract was washed with a saturated aqueous solution of sodium bicarbonate (60 mL) then with brine (60 mL), dried over sodium sulfate, filtered and concentrated to provide the title compound. The residue was used in the following step without further purification. MS (ESI) m/z: 316.1 [M+H]⁺.

### Step 2: 3,3-dichloro-1-(4-fluoro-1-methyl-1H-indazol-5-yl)piperidin-2-one

PCl₅ (7.8 g, 38 mmol) was added to a solution of 1-(4-fluoro-1-methyl-1H-indazol-5-yl)piperidin-2-one (3.1 g, 13 mmol) in chloroform (60 mL) at 65° C. The reaction mixture was stirred at the same temperature for 3 hours. The cooled reaction mixture was then slowly poured onto ice water (60 mL) and vigorously stirred while keeping the internal temperature below 20° C. After stirring for an additional 10 min, the mixture was extracted with DCM (60 mL). and the extract was washed with a saturated aqueous solution of sodium bicarbonate (60 mL) then with brine (60 mL), dried over sodium sulfate, filtered and concentrated to provide the title compound. The residue was used in the following step without further purification. MS (ESI) m/z: 316.1 [M+H]⁺.

### Step 3: 3,3-dichloro-1-(4-fluoro-1-methyl-1H-indazol-5-yl)piperidin-2-one

PCl₅ (7.8 g, 38 mmol) was added to a solution of 1-(4-fluoro-1-methyl-1H-indazol-5-yl)piperidin-2-one (3.1 g, 13 mmol) in chloroform (60 mL) at 65° C. The reaction mixture was stirred at the same temperature for 3 hours. The cooled reaction mixture was then slowly poured onto ice water (60 mL) and vigorously stirred while keeping the internal temperature below 20° C. After stirring for an additional 10 min, the mixture was extracted with DCM (60 mL) and the extract was washed with a saturated aqueous solution of sodium bicarbonate (60 mL) then with brine (60 mL), dried over sodium sulfate, filtered and concentrated to provide the title compound. The residue was used in the following step without further purification. MS (ESI) m/z: 316.1 [M+H]⁺.

### Step 4: 3-chloro-1-(4-fluoro-1-methyl-1H-indazol-5-yl)-5,6-dihydropyridin-2(1H)-one

Lithium carbonate (1.7 g, 0.82 mL, 23 mmol) and lithium chloride (0.50 g, 12 mmol) were sequentially added to a solution of 3,3-dichloro-1-(4-fluoro-1-methyl-1H-indazol-5-yl)piperidin-2-one (3.7 g, 12 mmol) in DMF (25 mL) at rt. The resulting mixture was heated to 135° C. and stirred for 4 hours. After cooling to room temperature, the mixture was quenched with H₂O (15 mL) and stirred for 5 min. The aqueous phase was extracted with EtOAc (20 mL×2), the combined organic layers were washed with a saturated aqueous solution of NaHCO₃ (20 mL×2) and brine (20 mL×2), dried over sodium sulfate, filtered and concentrated under reduced pressure to provide the title compound. The residue was used in the following step without further purification. MS (ESI) m/z: 279.9 [M+H]⁺.

### Step 5: 3-chloro-1-(4-fluoro-1-methyl-1H-indazol-5-yl)-5,6-dihydropyridin-2(1H)-one

Potassium acetate (2.05 g, 20.9 mmol) was added to a solution of 3-chloro-1-(4-fluoro-1-methyl-1H-indazol-5-yl)-5,6-dihydropyridin-2(1H)-one (1.95 g, 6.97 mmol) and bis(pinacolato)diborane (2.30 g, 9.06 mmol) in toluene (30 mL). The reaction mixture was sparged with argon for 10 min. BrettPhos Pd G3 (1.26 g, 1.39 mmol) was then added, and the reaction mixture was sparged with argon for an additional 5 min and stirred at 105° C. for 2 hours. The reaction mixture was used directly in the following step. MS (ESI) m/z: 372.3 [M+H]⁺.

### Step 6: tert-butyl (S)-3-(1-(4-fluoro-1-methyl-1H-indazol-5-yl)-2-oxo-1,2,5,6-tetrahydropyridin-3-yl)-2-(4-fluoro-3,5-dimethylphenyl)-4-methyl-2,4,6,7-tetrahydro-5H-pyrazolo[4,3-c]pyridine-5-carboxylate

A solution of potassium phosphate, tribasic (3.41 g, 16.1 mmol) in H₂O (3.5 mL) and tert-butyl (S)-3-bromo-2-(4-fluoro-3,5-dimethylphenyl)-4-methyl-2,4,6,7-tetrahydro-5H-pyrazolo[4,3-c]pyridine-5-carboxylate (2.35 g, 5.36 mmol) were sequentially added to the solution of 1-(4-fluoro-1-methyl-1H-indazol-5-yl)-3-(4,4,5,5-tetramethyl-1,3,2-dioxaborolan-2-yl)-5,6-dihydropyridin-2(1H)-one (2.59 g, 6.97 mmol) in toluene (30 mL) obtained in the previous step. The reaction mixture was sparged with argon for 10 min and BrettPhos Pd G3 (0.972 g, 1.07 mmol) was then added. The reaction mixture was again sparged with argon for 5 min and stirred at 105° C. for 1 hour. After cooling to rt, the reaction mixture was filtered through a Celite® pad and washed with DCM (35 mL×3). The volatiles were removed under reduced pressure and the residue was purified by flash chromatography (ISCO Combiflash, UV detection @254 nm) eluting with an increasing proportion of EtOAc (0 to 40%) in hexanes to give the title compound. MS (ESI) m/z: 603.3 [M+H]+.

### Step 7: tert-butyl (4S)-3-(3-(4-fluoro-1-methyl-1H-indazol-5-yl)-2-oxo-3-azabicyclo[4.1.0]heptan-1-yl)-2-(4-fluoro-3,5-dimethylphenyl)-4-methyl-2,4,6,7-tetrahydro-5H-pyrazolo[4,3-c]pyridine-5-carboxylate

Trimethyl(oxo)sulfonium iodide (3 g, 0.01 mol) was added to a suspension of sodium hydride (0.60 g, 60% wt, 10 mmol) in DMSO (20 mL) at room temperature. The reaction mixture was stirred for 30 min at the same temperature. A solution of tert-butyl (S)-3-(1-(4-fluoro-1-methyl-1H-indazol-5-yl)-2-oxo-1,2,5,6-tetrahydropyridin-3-yl)-2-(4-fluoro-3,5-dimethylphenyl)-4-methyl-2,4,6,7-tetrahydro-5H-pyrazolo[4,3-c]pyridine-5-carboxylate (3.2 g, 5.3 mmol) in DMSO (20 mL) was then added dropwise to the reaction mixture followed by stirring at 23° C. for 2 hours. The reaction mixture was poured into ice-water (15 mL), extracted with EtOAc (50 mL), washed with brine (25 mL×3), dried on MgSO₄ and concentrated under reduced pressure to provide a mixture of unassigned diastereomers (major:minor: 7.5/2.5). MS (ESI) m/z: 617.4 [M+H]⁺.

### Step 8: tert-butyl (S)-3-((1S,6S)-3-(4-fluoro-1-methyl-1H-indazol-5-yl)-2-oxo-3-azabicyclo[4.1.0]heptan-1-yl)-2-(4-fluoro-3,5-dimethylphenyl)-4-methyl-2,4,6,7-tetrahydro-5H-pyrazolo[4,3-c]pyridine-5-carboxylate

The diastereomeric mixture of tert-butyl (4S)-3-(3-(4-fluoro-1-methyl-1H-indazol-5-yl)-2-oxo-3-azabicyclo[4.1.0]heptan-1-yl)-2-(4-fluoro-3,5-dimethylphenyl)-4-methyl-2,4,6,7-tetrahydro-5H-pyrazolo[4,3-c]pyridine-5-carboxylate was separated by semi-preparative SFC (flow rate: 70 mL/min; sample concentration: 15 mg/mL; injection volume: 4000 μL; column: ChiralPak 1H 250×21.2 mm, 5 μm; co-solvent: methanol+0.1% NH₄OH (35%); outlet pressure: 100 bar; oven temperature: 40° C.; UV wavelength: 220 nm) collecting the fractions eluting. The fastest eluting was a major undesired and the second fasting eluting was a minor desired diastereomer. The purity of the desired diastereomer (minor) was determined by analytical SFC (sample concentration: 1 mg/mL; injection volume: 5.00 μL; column: i-Cellulose-5 IH 250×4.6 mm, 5 μm; co-solvent: methanol (35%); outlet pressure: 130 bar; oven temperature: 40° C.). Desired (RT 5.49 min) title compound, >99% de; LCMS purity: 96%. MS (ESI) m/z: 617.4 [M+H]⁺. ¹H NMR (CH₃OH-d₄, 400 MHz; Rotamers): δ_{H} 8.09 (s, 1H), 7.40 (d, J=8.9 Hz, 1H), 7.22 (s, 1H), 7.11 (d, J=6.2 Hz, 2H), 5.27 (s, 1H), 4.25 (d, J=10.7 Hz, 1H), 4.07 (d, J=9.8 Hz, 3H), 3.70-3.65 (m, 1H), 3.34 (s, 1H), 3.19 (s, 1H), 2.66-2.59 (m, 2H), 2.32 (s, 6H), 2.18 (s, 1H), 1.97 (d, J=13.7 Hz, 1H), 1.67 (s, 2H), 1.51 (d, J=16.2 Hz, 9H), 1.43 (d, J=6.4 Hz, 2H), 0.95-0.83 (m, 2H). ¹⁹F NMR (CH₃OH-d₄, 376 MHz; Rotamers): δ_{F} 122.4 (m, 1F), −126.8 (m, 1F).

### Step 9: (1S,6S)-3-(4-fluoro-1-methyl-1H-indazol-5-yl)-1-((S)-2-(4-fluoro-3,5-dimethylphenyl)-4-methyl-4,5,6,7-tetrahydro-2H-pyrazolo[4,3-c]pyridin-3-yl)-3-azabicyclo[4.1.0]heptan-2-one

tert-Butyl (4S)-3-(3-(4-fluoro-1-methyl-1H-indazol-5-yl)-2-oxo-3-azabicyclo[4.1.0]heptan-1-yl)-2-(4-fluoro-3,5-dimethylphenyl)-4-methyl-2,4,6,7-tetrahydro-5H-pyrazolo[4,3-c]pyridine-5-carboxylate (0.650 g, 1.05 mmol) was added to a solution of HCl in 1.4-dioxane (961 mg, 6.59 mL, 4 molar, 26.3 mmol) at rt followed by stirring for 1 hour at the same temperature. Et₂O (40 mL) was added and the precipitate was filtered and washed with Et₂O (20 mL×2) to provide the title compound. MS (ESI) m/z: 517.5 [M+H]⁺.

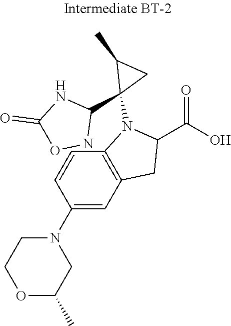 [SMILES: C[C@H]1CN(c2ccc3c(c2)CC(C(=O)O)N3[C@@]2(C3NOC(=O)N3)C[C@@H]2C)CCO1]

### 1-((1S,2S)-2-methyl-1-(5-oxo-4,5-dihydro-1,2,4-oxadiazol-3-yl)cyclopropyl)-5-((S)-2-methylmorpholino)-1H-indole-2-carboxylic acid

### Step 1: 1-((1S,2S)-2-methyl-1-(5-oxo-4,5-dihydro-1,2,4-oxadiazol-3-yl)cyclopropyl)-5-((S)-2-methylmorpholino)-1H-indole-2-carboxylic acid

(S)-2-methylmorpholine (742 mg, 7.34 mmol), 5-bromo-1-((1S,2S)-2-methyl-1-(5-oxo-4,5-dihydro-1,2,4-oxadiazol-3-yl)cyclopropyl)-1H-indole-2-carboxylic acid (1.85 g, 4.89 mmol), 2-Dicyclohexylphosphino-2′,6′-diisopropoxybiphenyl (457 mg, 978 mol), sodium tert-butoxide (1.88 g, 19.6 mmol) and Pd₂(dba)₃ (448 mg, 489 mol) were combined in a reaction tube. The reaction was brought into the glove box. NMP (24.5 mL) was added and the reaction tube sealed. The reaction was taken out of the glove box and stirred at 100° C. for 1 h. The reaction was diluted with EtOAc and water. The pH was adjusted to pH 2 with TFA. The aqueous layer was extracted 3x with EtOAc. Combined organic layers were dried with magnesium sulfate, filtered and concentrated. The residue still had ˜5 mL of NMP remaining. An additional 2 mL of DMSO was added and the residue purified directly by C18 reverse phase chromatography (10-100% MeCN/Water, with 0.1% TFA). Fractions were combined and extracted 3x with EtOAc. The combined organic layers were dried with magnesium sulfate, filtered and concentrated to give the title compound. MS (ESI) m/z: 399.4 [M+H]⁺.

The following intermediates in table BT were prepared using the procedures outlined in the synthesis of intermediate BT-2.

TABLE BT

| Intermediate | Structure | Name | MS (M + H) |
|---|---|---|---|
| BT-3 | 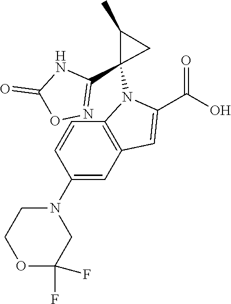 [SMILES: C[C@H]1C[C@]1(c1noc(=O)[nH]1)n1c(C(=O)O)cc2cc(N3CCOC(F)(F)C3)ccc21] | 5-(2,2-difluoromorpholino)-1- ((1S,2S)-2-methyl-1-(5-oxo-4,5- dihydro-1,2,4-oxadiazol-3- yl)cyclopropyl)-1H-indole-2- carboxylic acid | 421.2 |

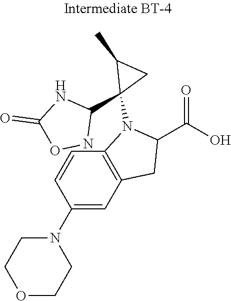 [SMILES: C[C@H]1C[C@]1(C1NOC(=O)N1)N1c2ccc(N3CCOCC3)cc2CC1C(=O)O]

### 1-((1S,2S)-2-methyl-1-(5-oxo-4,5-dihydro-1,2,4-oxadiazol-3-yl)cyclopropyl)-5-morpholino-1H-indole-2-carboxylic acid

### Step 1: (1R,2R)-8′-bromo-2-methyl-3′H,5′H-spiro[cyclopropane-1,12′-[1,2,4]oxadiazolo[4′,3′:4,5]pyrazino[1,2-a]indole]-3′,5′-dione

5-bromo-1-((1S,2S)-2-methyl-1-(5-oxo-4,5-dihydro-1,2,4-oxadiazol-3-yl)cyclopropyl)-1H-indole-2-carboxylic acid (5.0 g, 13.22 mmol) was combined with HATU (6.5 g, 17.2 mmol) and DIPEA (6.9 mL, 39.7 mmol) in DMF (66.1 mL). The reaction was stirred at 25° C. for 4 hour. Additional HATU (2.5 g, 6.6 mmol) and DIPEA (2.3 mL, 1 Eq, 13.22 mmol) was added and the reaction stirred at RT for 15 h. The reaction was diluted with water and EtOAc. The organic layer was washed with water 3x. The organic layer was dried with magnesium sulfate, filtered and concentrated. The residue was purified by silica gel chromatography (0-100% EtOAc in Hexanes). Fractions combined and concentrated to give the title compound. MS (ESI) m/z: 360.0 [M+H]⁺.

### Step 2: methyl 5-bromo-1-((1S,2S)-2-methyl-1-(5-oxo-4,5-dihydro-1,2,4-oxadiazol-3-yl)cyclopropyl)-1H-indole-2-carboxylate

Sodium methoxide (18.8 mL, 0.5 molar in MeOH, 9.41 mmol) was added to a mixture of (1R,2R)-8′-bromo-2-methyl-3′H,5′H-spiro[cyclopropane-1,12′-[1,2,4]oxadiazolo[4′,3′:4,5]pyrazino[1,2-a]indole]-3′,5′-dione (2.3 g, 6.27 mmol) in methanol (31.4 mL). The reaction mixture was allowed to stir at RT for 1 h. The reaction was added to a saturated ammonium chloride solution, then water was added (pH around 6.5) and the mixture was extracted with EtOAc 3x. The combined organic layers were dried with magnesium sulfate, filtered and concentrated. The residue was purified by silica gel chromatography (0-100% 3:1 EtOAc:EtOH in hexane). Fractions combined and concentrated to give the title compound. MS (ESI) m/z: 414.2 [M+Na]⁺.

### Step 3: methyl 1-((1S,2S)-2-methyl-1-(5-oxo-4,5-dihydro-1,2,4-oxadiazol-3-yl)cyclopropyl)-5-morpholino-1H-indole-2-carboxylate

Morpholine (53.4 μL, 620 mol), methyl 5-bromo-1-((1S,2S)-2-methyl-1-(5-oxo-4,5-dihydro-1,2,4-oxadiazol-3-yl)cyclopropyl)-1H-indole-2-carboxylate (0.162 g, 413 mol), Pd₂dba₃ (75.6 mg, 82.6 mol), cesium carbonate (404 mg, 1.24 mmol) and 2-dicyclohexylphosphino-2′,6′-diisopropoxybiphenyl (77.1 mg, 165 mol) were combined in a sealed vial which was then purged with nitrogen gas. Next, 1,4-Dioxane (8.26 mL) added, and the reaction purged with nitrogen gas. Then the reaction was heated to 100° C. for 16 hour after which the reaction mixture was filtered and concentrated. The residue was diluted with 2 mL of DMSO, filtered and purified by reverse phase chromatography (10-100% MeCN/water with 0.1% TFA). Fractions were combined and extracted with EtOAc 3x. The combined organic layers were dried with magnesium sulfate, filtered and concentrated to give the title compound. MS (ESI) m/z: 399.4 [M+H]⁺.

### Step 4: 1-((1S,2S)-2-methyl-1-(5-oxo-4,5-dihydro-1,2,4-oxadiazol-3-yl)cyclopropyl)-5-morpholino-1H-indole-2-carboxylic acid

Methyl 1-((1S,2S)-2-methyl-1-(5-oxo-4,5-dihydro-1,2,4-oxadiazol-3-yl)cyclopropyl)-5-morpholino-1H-indole-2-carboxylate (0.080 g, 0.20 mmol) and sodium hydroxide (0.60 mL, 1 molar solution in water, 0.60 mmol) were combined in DMSO (1.0 mL). The reaction was stirred at 25° C. for 15 hour. The reaction was loaded directly onto a reverse phase column (10-100% MeCN/water with 0.1% TFA). Fractions were combined and extracted 3× with EtOAc. The combined organic layers were dried with magnesium sulfate, filtered and concentrated to give the title compound. MS (ESI) m/z: 385.4 [M+H]+.

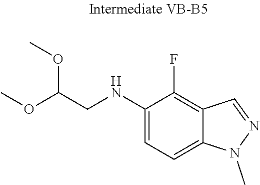 [SMILES: COC(CNc1ccc2c(cnn2C)c1F)OC]

### N-(2,2-dimethoxyethyl)-4-fluoro-1-methyl-1H-indazol-5-amine

A mixture of 2,2-dimethoxyacetaldehyde (60% wt in water, 7.18 g, 6.24 mL, 41.4 mmol) in MeOH (63 mL) was treated sequentially with 4-fluoro-1-methyl-1H-indazol-5-aminium chloride (4.17 g, 20.7 mmol) and sodium acetate (1.87 g, 22.8 mmol). The mixture was stirred vigorously for 30 min. The mixture was then cooled to 0° C., and sodium cyanoborohydride (3.90 g, 62.0 mmol) was added. The mixture was warmed to room temperature and stirred for 16 h. The mixture was then quenched by addition of saturated aqueous NaHCO₃ (20 mL) and partially concentrated under reduced pressure. The remaining aqueous mixture was shaken with EtOAc (45 mL). After separation, the aqueous layer was back-extracted with EtOAc (2×45 mL). The combined organic layers were washed with brine (25 mL), dried over MgSO₄, filtered and concentrated under reduced pressure. The residue was purified via C18 reverse phase column chromatography (10-100% MeCN/water) to provide the title compound. ¹HNMR (CDCl₃, 400 MHz): δ 7.90 (1H, s), 7.01-7.07 (2H, m), 4.54 (1H, t, J=5.5 Hz), 4.02 (3H, s), 3.43 (6H, s), 3.33 (2H, d, J=5.5 Hz). MS (ESI) m/z: 222.2 [M−MeOH+H]⁺.

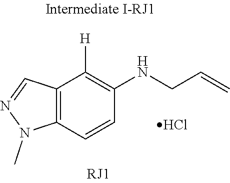

### tert-butyl allyl(4-fluoro-1-methyl-1H-indazol-5-yl)carbamate

### Step 1: tert-butyl (4-fluoro-1-methyl-1H-indazol-5-yl)carbamate

Tert-butyl (4-fluoro-1-methyl-1H-indazol-5-yl)carbamate (1.0 g, 4.0 mmol) was added to a suspension of sodium hydride (0.2 g, 60% wt, 4 mmol) in THF (12 mL) at 0° C. The reaction mixture was stirred for 1 hour at the same temperature. A solution of allyl bromide (0.5 mL, 6 mmol) in THF (13 mL) was then added dropwise. The cooling bath was then removed, and the mixture was stirred at 25° C. for 16 hours. The reaction mixture was then poured into ice-water (15 mL) and extracted with EtOAc (25 mL). The organic layer was washed with brine (15 mL×3), dried over MgSO₄, filtered, and then concentrated under reduced pressure. The crude residue was purified by silica gel chromatography (hexanes/EtOAc: 1/4 to 0/1) to provide the title compound. LCMS m/z[M+H]:306.1

### Step 2: N-allyl-4-fluoro-1-methyl-1H-indazol-5-ammonium chloride

To a solution of tert-butyl allyl(4-fluoro-1-methyl-1H-indazol-5-yl)carbamate (980 mg, 3.21 mmol) in 1,4-dioxane was added HCl (12.0 mL, 48.1 mmol, 4 M in 1,4-dioxane). The reaction mixture was stirred for 1 hour at 23° C. Et₂O (20 mL) was added, and the resultant precipitate was filtered and washed with Et₂O (10 mL×2) to provide the title compound without further purification. LCMS m/z[M+H]: 206.2.

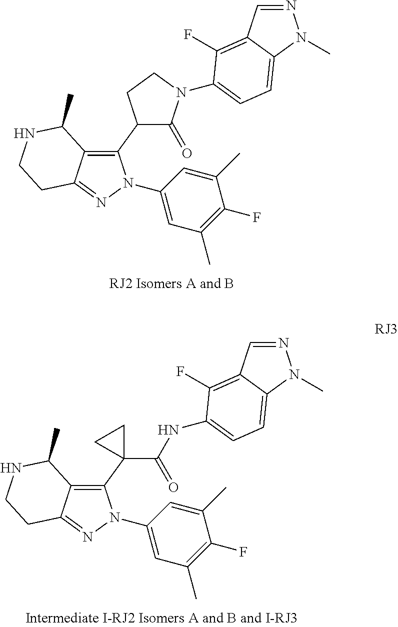 [SMILES: Cc1cc(-n2nc3c(c2C2CCN(c4ccc5c(cnn5C)c4F)C2=O)[C@H](C)NCC3)cc(C)c1F ; Cc1cc(-n2nc3c(c2C2(C(=O)Nc4ccc5c(cnn5C)c4F)CC2)[C@H](C)NCC3)cc(C)c1F]

### Step 1: tert-butyl (S)-3-((S)-1-(4-fluoro-1-methyl-1H-indazol-5-yl)-2-oxopyrrolidin-3-yl)-2-(4-fluoro-3,5-dimethylphenyl)-4-methyl-2,4,6,7-tetrahydro-5H-pyrazolo[4,3-c]pyridine-5-carboxylate and tert-butyl (S)-3-((R)-1-(4-fluoro-1-methyl-1H-indazol-5-yl)-2-oxopyrrolidin-3-yl)-2-(4-fluoro-3,5-dimethylphenyl)-4-methyl-2,4,6,7-tetrahydro-5H-pyrazolo[4,3-c]pyridine-5-carboxylate and tert-butyl (S)-3-(1-((4-fluoro-1-methyl-1H-indazol-5-yl)carbamoyl)cyclopropyl)-2-(4-fluoro-3,5-dimethylphenyl)-4-methyl-2,4,6,7-tetrahydro-5H-pyrazolo[4,3-c]pyridine-5-carboxylate

To a stirred solution of trimethyl(oxo)sulfonium iodide (126 mg, 572 mol), 1-methyl-2,3,4,6,7,8-hexahydro-1H-pyrimido[1,2-a]pyrimidine (104 mg, 97.1 L, 676 mol) in MeCN (1 mL) was added tert-butyl (S)-3-(3-((4-fluoro-1-methyl-1H-indazol-5-yl)amino)-3-oxoprop-1-en-2-yl)-2-(4-fluoro-3,5-dimethylphenyl)-4-methyl-2,4,6,7-tetrahydro-5H-pyrazolo[4,3-c]pyridine-5-carboxylate (300 mg, 520 mol). The reaction mixture was heated to 60° C. and stirred for 1 hour. The mixture was cooled to room temperature, concentrated under reduced pressure, and redissolved in DCM. The crude solution was directly purified by silica gel chromatography (hexanes/EtOAc: 1/0 to 0/1) to provide the title compounds. Intermediate A Isomer A: LCMS m/z[M+H]: 591.2. Intermediate A Isomer B: LCMS m/z[M+H]: 591.2. Intermediate B: LCMS m/z[M+H]: 591.2

### Step 2: (S)-1-(4-fluoro-1-methyl-1H-indazol-5-yl)-3-((S)-2-(4-fluoro-3,5-dimethylphenyl)-4-methyl-4,5,6,7-tetrahydro-2H-pyrazolo[4,3-c]pyridin-3-yl)pyrrolidin-2-one and (R)-1-(4-fluoro-1-methyl-1H-indazol-5-yl)-3-((S)-2-(4-fluoro-3,5-dimethylphenyl)-4-methyl-4,5,6,7-tetrahydro-2H-pyrazolo[4,3-c]pyridin-3-yl)pyrrolidin-2-one

To a solution of either (S)-3-((S)-1-(4-fluoro-1-methyl-1H-indazol-5-yl)-2-oxopyrrolidin-3-yl)-2-(4-fluoro-3,5-dimethylphenyl)-4-methyl-2,4,6,7-tetrahydro-5H-pyrazolo[4,3-c]pyridine-5-carboxylate or tert-butyl (S)-3-((R)-1-(4-fluoro-1-methyl-1H-indazol-5-yl)-2-oxopyrrolidin-3-yl)-2-(4-fluoro-3,5-dimethylphenyl)-4-methyl-2,4,6,7-tetrahydro-5H-pyrazolo[4,3-c]pyridine-5-carboxylate (51.3 mg, 86.8 mol) in DCM (868 L) was added HCl (217 μL, 868 mol, 4.00 M in 1,4-dioxane). The resultant mixture was stirred at 20° C. for 18 hours. The crude reaction mixture was concentrated and used in the next step without purification. LCMS m/z[M+H]:491.4

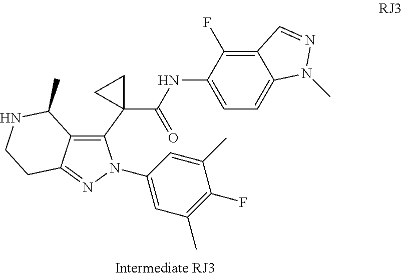 [SMILES: Cc1cc(-n2nc3c(c2C2(C(=O)Nc4ccc5c(cnn5C)c4F)CC2)[C@H](C)NCC3)cc(C)c1F]

### (S)—N-(4-fluoro-1-methyl-1H-indazol-5-yl)-1-(2-(4-fluoro-3,5-dimethylphenyl)-4-methyl-4,5,6,7-tetrahydro-2H-pyrazolo[4,3-c]pyridin-3-yl)cyclopropane-1-carboxamide

The procedure to synthesize RJ3 follows the same preparation as demonstrated for Intermediate RJ2. LCMS m/z[M+H]:491.2

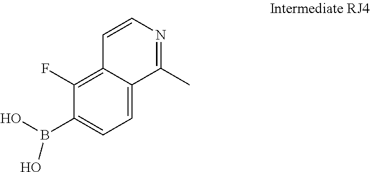

### Step 1: 1,6-dibromo-5-fluoroisoquinoline

To a solution of 6-bromo-5-fluoroisoquinolin-1-ol (1.5 g, 6.2 mmol) in EtOAc (30 mL) was added phosphoryl tribromide (7.1 g, 25 mmol), the resultant mixture was stirred at 15° C. for 20 hours. The pH was adjusted to 8 using saturated sodium bicarbonate (50 mL) and extracted with tetrahydrofuran (3×30 mL). The combined organic layers were washed with brine (50 mL), dried over anhydrous sodium sulfate, filtered, and concentrated under reduced pressure. The residue was purified by C18 reverse phase column chromatography (55-85% MeCN/water+0.1% FA modifier) to afford the title compound. LCMS m/z[M+H]: 305.7

### Step 2: 6-bromo-5-fluoro-1-methylisoquinoline

To a solution of 1,6-dibromo-5-fluoroisoquinoline (1.45 g, 4.76 mmol) and 2,4,4,5,5-pentamethyl-1,3,2-dioxaborolane (1.22 g, 8.56 mmol) in 1,4-dioxane (15 mL) and H₂O (3.0 mL) was added potassium phosphate, tribasic (4.0 g, 19 mmol) and 1,1′-bis(diphenylphosphino) ferrocene-palladium(II) dichloride (348 mg, 476 mol). The mixture was stirred at 80° C. for 16 hours. The reaction mixture was diluted with water (20 mL) and extracted with ethyl acetate (3×20 mL). The combined organic layers were dried over anhydrous sodium sulfate and filtered. The filtrate was concentrated under reduced pressure and purified by silica gel chromatography (hexanes/EtOAc: 1/0 to 65/35) to provide the title compound. LCMS m/z[M+H]: 239.8

### Step 3: (5-fluoro-1-methylisoquinolin-6-yl)boronic acid

To a solution of 6-bromo-5-fluoro-1-methylisoquinoline (380 mg, 1.58 mmol) and 4,4,4′,4′,5,5,5′,5′-octamethyl-2,2′-bi(1,3,2-dioxaborolane) (603 mg, 2.37 mmol) in 1,4-dioxane (1 mL) was added potassium acetate (621 mg, 6.33 mmol) and 1,1′-bis(diphenylphosphino) ferrocene-palladium(II) dichloride (115 mg, 158 mol). The mixture was stirred at 80° C. for 16 hours under a N₂ atmosphere. The reaction mixture was concentrated under reduced pressure to afford crude, which was purified by C18 reverse phase column chromatography (0-15% MeCN/water+0.1% TFA modifier) to provide the title compound. LCMS m/z[M+H]: 206.0.

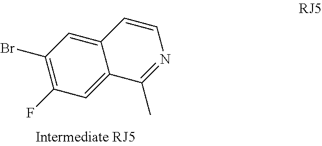

### Step 1: 6-bromo-7-fluoro-1-methylisoquinoline

A mixture of iron (III) (Z)-4-oxopent-2-en-2-olate (13.6 mg, 38.4 mol), N1,N1,N2,N2-tetramethylethane-1,2-diamine (44.6 mg, 57.6 μL, 384 mol) and 6-bromo-1-chloro-7-fluoroisoquinoline (200 mg, 768 mol) were combined in THF (6 mL). The resultant mixture was allowed to stir for 10 minutes at 0° C. under nitrogen. To this mixture was added methylmagnesium bromide (101 mg, 282 μL, 3 M, 845 mol) dropwise under nitrogen and the mixture was stirred for 2 hours at 0° C. A 5% aqueous solution of citric acid (10 mL) was added at 0° C. and the organic phase was separated. The organic phase was washed with 5% aqueous solution of citric acid (10 mL), then washed with brine (10 mL), and finally dried over sodium sulfate to afford the crude residue which was purified by prep-TLC(EtOAc) to provide the title compound. MS(ESI)m/z: 241.9 [M+H]⁺.

### EXAMPLES

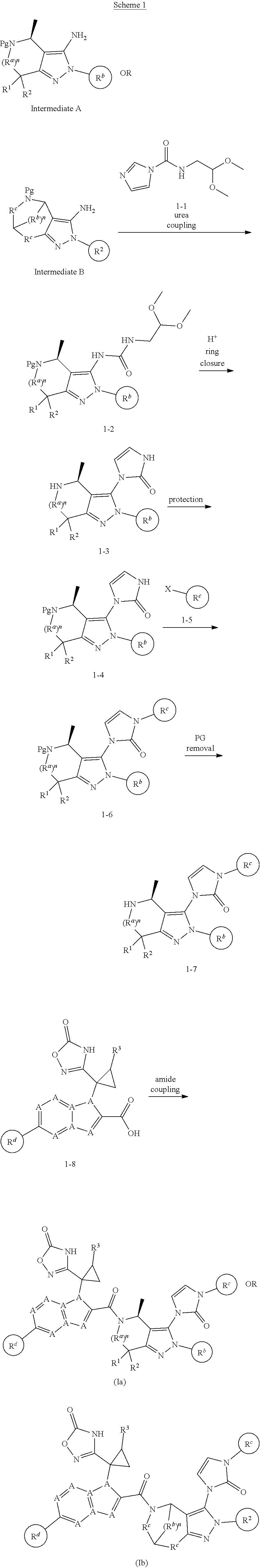

In Schemes 1-16, R^{c} is C₅-C₁₂ aryl, C₅-C₁₂ heteroaryl, alkyl, cycloalkyl, or heterocycle; R^{d}=alkyl, cycloalkyl, heterocycle, aryl, or heteroaryl; and R^{e} is C₅-C₁₂ aryl, C₅-C₁₂ heteroaryl. The remaining variables in Schemes 1-16 and would be understood by a person of ordinary skill in the art in view of their own knowledge and the below examples. The variables the Schemes herein (1-18 and VB-A-VbQ) are separate from the definitions of these variables in the summary, detailed description and claims of the present application.

Compounds of formula (Ia) are prepared from intermediate A by coupling with N-(2,2-dimethoxyethyl)-1H-imidazole-1-carboxamide (1-1) under basic conditions to provide urea 1-2. Ring closure of 1-2 promoted by acidic conditions provides imidazol-2-one 1-3. Protection of the amine group of 1-3 provides intermediate 1-4. Alternatively, in some examples, ring closure of 1-2 provides 1-4 directly, without the protection step. Subsequently, 1-4 is coupled with halides or other partners such as boronic acids or sulfonates (1-5) via transition metal catalyzed cross-coupling (e.g., Pd- or Cu-catalysis) or alkylation (e.g., basic) conditions to furnish intermediate 1-6. Removal of the protecting group (e.g., acidic conditions) of 1-6 provides amine 1-7 that is subsequently coupled with carboxylic acid 1-8 to provide compounds of formula (I). Alternatively, the identical sequence of steps starting with intermediate B provides compounds of formula (Ib).

### Example 1

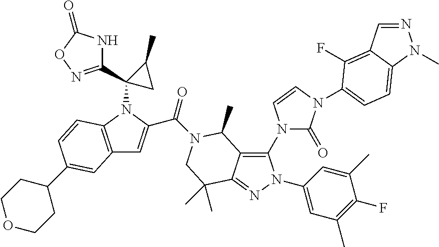 [SMILES: Cc1cc(-n2nc3c(c2-n2ccn(-c4ccc5c(cnn5C)c4F)c2=O)[C@H](C)N(C(=O)c2cc4cc(C5CCOCC5)ccc4n2[C@@]2(c4noc(=O)[nH]4)C[C@@H]2C)CC3(C)C)cc(C)c1F]

### 3-((1S,2S)-1-(2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4,7,7-trimethyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one (Scheme 1)

### Step 1: tert-butyl (S)-3-(3-(2,2-dimethoxyethyl)ureido)-2-(4-fluoro-3,5-dimethylphenyl)-4,7,7-trimethyl-2,4,6,7-tetrahydro-5H-pyrazolo[4,3-c]pyridine-5-carboxylate

To a mixture of tert-butyl (S)-3-amino-2-(4-fluoro-3,5-dimethylphenyl)-4,7,7-trimethyl-2,4,6,7-tetrahydro-5H-pyrazolo[4,3-c]pyridine-5-carboxylate (1.0 g, 2.5 mmol) and N-(2,2-dimethoxyethyl)-1H-imidazole-1-carboxamide (1.1 g, 5.5 mmol) in DMA (20 mL) at 25° C. was added potassium 2-methylpropan-2-olate (2.0 g, 17 mmol). The reaction mixture was stirred at 25° C. for 2 h. Upon reaction completion, the mixture was quenched with H₂O (20 mL) and extracted with EtOAc (3×20 mL). The combined organic layers were washed with brine (50 mL), dried over Na₂SO₄, filtered, and concentrated under reduced pressure. The crude mixture was purified by silica gel chromatography (0 to 70% EtOAc/Pet.ether) to provide the title compound. MS (ESI) m/z 534.2.

### Step 2: tert-butyl (S)-2-(4-fluoro-3,5-dimethylphenyl)-4,7,7-trimethyl-3-(2-oxo-2,3-dihydro-1H-imidazol-1-yl)-2,4,6,7-tetrahydro-5H-pyrazolo[4,3-c]pyridine-5-carboxylate

To a solution of tert-butyl (S)-3-(3-(2,2-dimethoxyethyl)ureido)-2-(4-fluoro-3,5-dimethylphenyl)-4,7,7-trimethyl-2,4,6,7-tetrahydro-5H-pyrazolo[4,3-c]pyridine-5-carboxylate (660 mg, 1.24 mmol) in THF (10 mL) at rt was added methanesulfonic acid (96 mg, 1.0 mmol). The reaction mixture was stirred at 60° C. for 2 h. Upon reaction completion, K₃PO₄ (saturated aqueous solution) was added at 0° C. to adjust the pH of the mixture to approx. 8 to 9, then the resultant mixture was extracted with EtOAc (3×15 mL). The combined organic layers were washed with brine (20 mL), dried over Na₂SO₄, filtered, and concentrated under reduced pressure to provide the title compound. MS (ESI) m/z 470.1

### Step 3: tert-butyl (S)-3-(3-(4-fluoro-1-methyl-1H-indazol-5-yl)-2-oxo-2,3-dihydro-1H-imidazol-1-yl)-2-(4-fluoro-3,5-dimethylphenyl)-4,7,7-trimethyl-2,4,6,7-tetrahydro-5H-pyrazolo[4,3-c]pyridine-5-carboxylate

To a mixture of 5-bromo-4-fluoro-1-methyl-1H-indazole (290 mg, 1.28 mmol), tert-butyl (S)-2-(4-fluoro-3,5-dimethylphenyl)-4,7,7-trimethyl-3-(2-oxo-2,3-dihydro-1H-imidazol-1-yl)-2,4,6,7-tetrahydro-5H-pyrazolo[4,3-c]pyridine-5-carboxylate (300 mg, 0.639 mmol), potassium phosphate tribasic (407 mg, 1.92 mmol) and (1R,2R)—N1,N2-dimethylcyclohexane-1,2-diamine (54.5 mg, 0.383 mmol) in dioxane (6.0 mL) in an inert atmosphere glove box was added copper(I) iodide (36.5 mg, 0.192 mmol). The mixture was stirred at 100° C. for 2.5 h. Then, the mixture was cooled to rt, filtered and the filtrate was quenched with H₂O (10 mL) and extracted with EtOAc (3×10 mL). The combined organic layers were washed with brine (20 mL), dried over Na₂SO₄, filtered, and concentrated under reduced pressure. The residue was purified by silica gel chromatography (0 to 40% EtOAc/Pet.ether) to provide the title compound. ¹H NMR (400 MHz, methanol-d4) δ 8.22 (s, 1H), 7.59-7.53 (m, 1H), 7.50-7.41 (m, 1H), 7.19 (d, J=6.4 Hz, 2H), 6.90 (d, J=3.2 Hz, 1H), 6.78 (s, 1H), 5.44-5.29 (m, 1H), 4.09-3.95 (m, 1H), 2.33 (d, J=2.0 Hz, 7H), 1.56 (s, 9H), 1.46 (s, 3H). MS (ESI) 618.2.

### Step 4: (S)-1-(4-fluoro-1-methyl-1H-indazol-5-yl)-3-(2-(4-fluoro-3,5-dimethylphenyl)-4,7,7-trimethyl-4,5,6,7-tetrahydro-2H-pyrazolo[4,3-c]pyridin-3-yl)-1,3-dihydro-2H-imidazol-2-one hydrochloride

To a solution of tert-butyl (S)-3-(3-(4-fluoro-1-methyl-1H-indazol-5-yl)-2-oxo-2,3-dihydro-1H-imidazol-1-yl)-2-(4-fluoro-3,5-dimethylphenyl)-4,7,7-trimethyl-2,4,6,7-tetrahydro-5H-pyrazolo[4,3-c]pyridine-5-carboxylate (200 mg, 0.32 mmol) in dioxane (0.50 mL) was added 2M HCl in dioxane (0.16 mL, 0.32 mmol), and the reaction mixture was stirred for 2 h at 25° C. Then, the reaction mixture was concentrated under reduced pressure to provide the title compound. MS (ESI) m/z 518.2.

### Step 5: 3-((1S,2S)-1-(2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4,7,7-trimethyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one

To a mixture of 1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carboxylic acid (89 mg, 0.23 mmol), (S)-1-(4-fluoro-1-methyl-1H-indazol-5-yl)-3-(2-(4-fluoro-3,5-dimethylphenyl)-4,7,7-trimethyl-4,5,6,7-tetrahydro-2H-pyrazolo[4,3-c]pyridin-3-yl)-1,3-dihydro-2H-imidazol-2-one, HCl (130 mg, 0.23 mmol) and HATU (130 mg, 0.35 mmol) in DMF (3.0 mL) was added DIPEA (0.16 mL, 0.93 mmol) at 0° C. The reaction mixture was stirred for 16 h at 25° C. then concentrated under reduced pressure. The crude residue was purified by prep-HPLC (57 to 87% MeCN/H₂O+0.1 TFA gradient) to provide the title compound as a TFA salt. ¹H NMR (400 MHz, DMSO-d6) δ 12.31-11.67 (m, 1H), 8.32-8.23 (m, 1H), 7.65-7.58 (m, 1H), 7.52-7.44 (m, 1H), 7.44-7.24 (m, 3H), 7.19-7.12 (m, 2H), 7.10-6.94 (m, 2H), 6.87 (d, J=1.6 Hz, 1H), 5.83-5.50 (m, 1H), 4.15-4.07 (m, 4H), 3.99-3.93 (m, 2H), 3.48-3.43 (m, 2H), 2.88-2.80 (m, 1H), 2.25 (s, 6H), 2.14-1.84 (m, 1H), 1.79-1.65 (m, 7H), 1.55-1.30 (m, 9H), 1.25-1.15 (m, 3H). MS (ESI) m/z 883.4. The following examples in table 1a were prepared according to scheme 1 using the procedures outlined in the synthesis of example 1. In cases wherein isomers of the examples were separated via chiral chromatography, conditions are included, and the faster eluting isomer is listed first.

TABLE la

| | | | MS |
|---|---|---|---|
| | | | (M + |
| Ex. | Structure | Name | 1) |
| 2 | 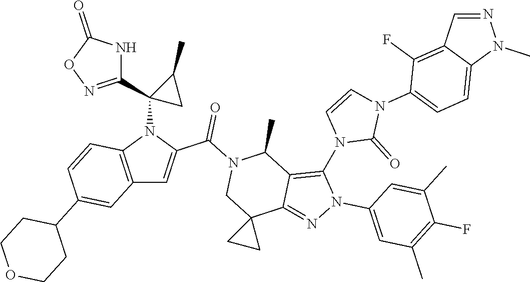 [SMILES: Cc1cc(-n2nc3c(c2-n2ccn(-c4ccc5c(cnn5C)c4F)c2=O)[C@H](C)N(C(=O)c2cc4cc(C5CCOCC5)ccc4n2[C@@]2(c4noc(=O)[nH]4)C[C@@H]2C)CC32CC2)cc(C)c1F] | 3-((1S,2S)-1-(2-((S)-3′-(3-(4- fluoro-1-methyl-1H-indazol-5- yl)-2-oxo-2,3-dihydro-1H- imidazol-1-yl)-2-(4-fluoro-3,5- dimethylphenyl)-4′-methyl- 2′,4′,5′,6′- tetrahydrospiro[cyclopropane- 1,7′-pyrazolo[4,3-c]pyridine]-5′- carbonyl)-5-(tetrahydro-2H- pyran-4-yl)-1H-indol-1-yl)-2- methylcyclopropyl)-1,2,4- oxadiazol-5(4H)-one | 881.3 |
| 3 | 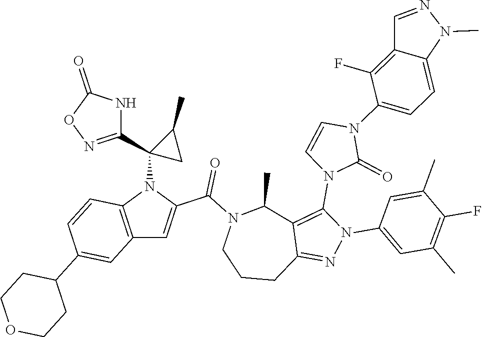 [SMILES: Cc1cc(-n2nc3c(c2-n2ccn(-c4ccc5c(cnn5C)c4F)c2=O)[C@H](C)N(C(=O)c2cc4cc(C5CCOCC5)ccc4n2[C@@]2(c4noc(=O)[nH]4)C[C@@H]2C)CCC3)cc(C)c1F] | 3-((1S,2S)-1-(2-((S)-3-(3-(4- fluoro-1-methyl-1H-indazol-5- yl)-2-oxo-2,3-dihydro-1H- imidazol-1-yl)-2-(4-fluoro-3,5- dimethylphenyl)-4-methyl- 2,4,5,6,7,8- hexahydropyrazolo[4,3- c]azepine-5-carbonyl)-5- (tetrahydro-2H-pyran-4-yl)-1H- indol-1-yl)-2-methylcyclopropyl)- 1,2,4-oxadiazol-5(4H)-one | 869.2 |
| 4 | 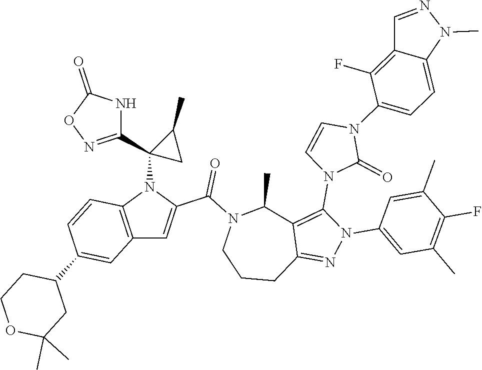 | 3-((1S,2S)-1-(5-((S)-2,2- dimethyltetrahydro-2H-pyran-4- yl)-2-((S)-3-(3-(4-fluoro-1- methyl-1H-indazol-5-yl)-2-oxo- 2,3-dihydro-1H-imidazol-1-yl)-2- (4-fluoro-3,5-dimethylphenyl)-4- methyl-2,4,5,6,7,8- hexahydropyrazolo[4,3- c]azepine-5-carbonyl)-1H-indol- 1-yl)-2-methylcyclopropyl)-1,2,4- oxadiazol-5(4H)-one | 897.3 |
| 5 | 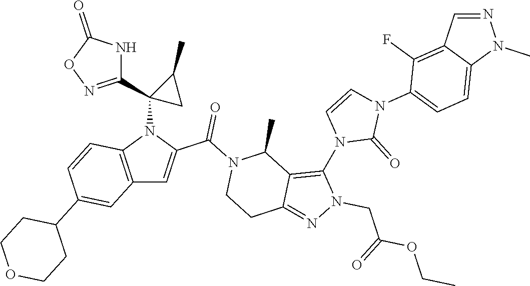 [SMILES: CCOC(=O)Cn1nc2c(c1-n1ccn(-c3ccc4c(cnn4C)c3F)c1=O)[C@H](C)N(C(=O)c1cc3cc(C4CCOCC4)ccc3n1[C@@]1(c3noc(=O)[nH]3)C[C@@H]1C)CC2] | ethyl 2-((S)-3-(3-(4-fluoro-1- methyl-1H-indazol-5-yl)-2-oxo- 2,3-dihydro-1H-imidazol-1-yl)-4- methyl-5-(1-((1S,2S)-2-methyl-1- (5-oxo-4,5-dihydro-1,2,4- oxadiazol-3-yl)cyclopropyl)-5- (tetrahydro-2H-pyran-4-yl)-1H- indole-2-carbonyl)-4,5,6,7- tetrahydro-2H-pyrazolo[4,3- c]pyridin-2-yl)acetate | 819.3 |
| 6 | 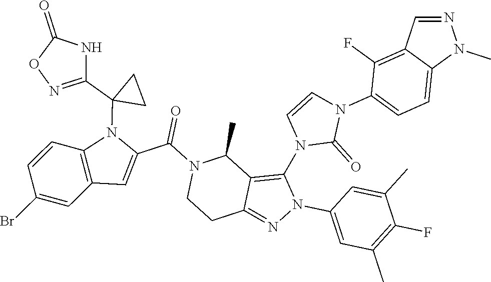 [SMILES: Cc1cc(-n2nc3c(c2-n2ccn(-c4ccc5c(cnn5C)c4F)c2=O)[C@H](C)N(C(=O)c2cc4cc(Br)ccc4n2C2(c4noc(=O)[nH]4)CC2)CC3)cc(C)c1F] | (S)-3-(1-(5-bromo-2-(3-(3-(4- fluoro-1-methyl-1H-indazol-5- yl)-2-oxo-2,3-dihydro-1H- imidazol-1-yl)-2-(4-fluoro-3,5- dimethylphenyl)-4-methyl- 4,5,6,7-tetrahydro-2H- pyrazolo[4,3-c]pyridine-5- carbonyl)-1H-indol-1- yl)cyclopropyl)-1,2,4-oxadiazol- 5(4H)-one | 837.4 |
| 7 | 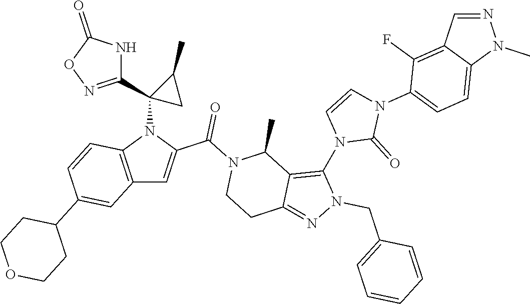 [SMILES: C[C@H]1c2c(nn(Cc3ccccc3)c2-n2ccn(-c3ccc4c(cnn4C)c3F)c2=O)CCN1C(=O)c1cc2cc(C3CCOCC3)ccc2n1[C@@]1(c2noc(=O)[nH]2)C[C@@H]1C] | 3-((1S,2S)-1-(2-((S)-2-benzyl-3- (3-(4-fluoro-1-methyl-1H- indazol-5-yl)-2-oxo-2,3-dihydro- 1H-imidazol-1-yl)-4-methyl- 4,5,6,7-tetrahydro-2H- pyrazolo[4,3-c]pyridine-5- carbonyl)-5-(tetrahydro-2H- pyran-4-yl)-1H-indol-1-yl)-2- methylcyclopropyl)-1,2,4- oxadiazol-5(4H)-one | 823.1 |
| 8 | 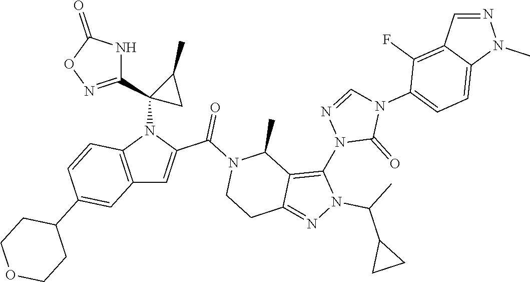 [SMILES: CC(C1CC1)n1nc2c(c1-n1ncn(-c3ccc4c(cnn4C)c3F)c1=O)[C@H](C)N(C(=O)c1cc3cc(C4CCOCC4)ccc3n1[C@@]1(c3noc(=O)[nH]3)C[C@@H]1C)CC2] | 3-((1S,2S)-1-(2-((S)-2-((S)-1- cyclopropylethyl)-3-(3-(4-fluoro- 1-methyl-1H-indazol-5-yl)-2-oxo- 2,3-dihydro-1H-imidazol-1-yl)-4- methyl-4,5,6,7-tetrahydro-2H- pyrazolo[4,3-c]pyridine-5- carbonyl)-5-(tetrahydro-2H- pyran-4-yl)-1H-indol-1-yl)-2- methylcyclopropyl)-1,2,4- oxadiazol-5(4H)-one and 3- ((1S,2S)-1-(2-((S)-2-((R)-1- cyclopropylethyl)-3-(3-(4-fluoro- 1-methyl-1H-indazol-5-yl)-2-oxo- 2,3-dihydro-1H-imidazol-1-yl)-4- methyl-4,5,6,7-tetrahydro-2H- pyrazolo[4,3-c]pyridine-5- carbonyl)-5-(tetrahydro-2H- | 801.6 |
| | | pyran-4-yl)-1H-indol-1-yl)-2- | |
| | | methylcyclopropyl)-1,2,4- | |
| | | oxadiazol-5(4H)-one | |
| 9 | 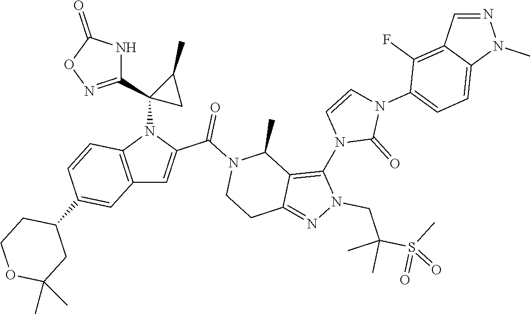 [SMILES: C[C@H]1c2c(nn(CC(C)(C)S(C)(=O)=O)c2-n2ccn(-c3ccc4c(cnn4C)c3F)c2=O)CCN1C(=O)c1cc2cc([C@H]3CCOC(C)(C)C3)ccc2n1[C@@]1(c2noc(=O)[nH]2)C[C@@H]1C] | 3-((1S,2S)-1-(5-((S)-2,2- dimethyltetrahydro-2H-pyran-4- yl)-2-((S)-3-(3-(4-fluoro-1- methyl-1H-indazol-5-yl)-2-oxo- 2,3-dihydro-1H-imidazol-1-yl)-4- methyl-2-(2-methyl-2- (methylsulfonyl)propyl)-4,5,6,7- tetrahydro-2H-pyrazolo[4,3- c]pyridine-5-carbonyl)-1H-indol- 1-yl)-2-methylcyclopropyl)-1,2,4- oxadiazol-5(4H)-one | 895.7 |
| 10 | 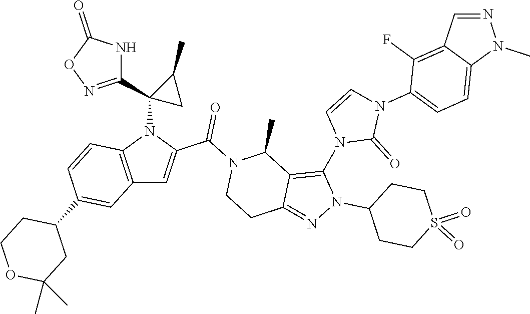 [SMILES: C[C@H]1c2c(nn(C3CCS(=O)(=O)CC3)c2-n2ccn(-c3ccc4c(cnn4C)c3F)c2=O)CCN1C(=O)c1cc2cc([C@H]3CCOC(C)(C)C3)ccc2n1[C@@]1(c2noc(=O)[nH]2)C[C@@H]1C] | 3-((1S,2S)-1-(5-(2,2- dimethyltetrahydro-2H-pyran-4- yl)-2-((S)-2-(1,1- dioxidotetrahydro-2H-thiopyran- 4-yl)-3-(3-(4-fluoro-1-methyl-1H- indazol-5-yl)-2-oxo-2,3-dihydro- 1H-imidazol-1-yl)-4-methyl- 4,5,6,7-tetrahydro-2H- pyrazolo[4,3-c]pyridine-5- carbonyl)-1H-indol-1-yl)-2- methylcyclopropyl)-1,2,4- oxadiazol-5(4H)-one | 893.4 |
| 11 | 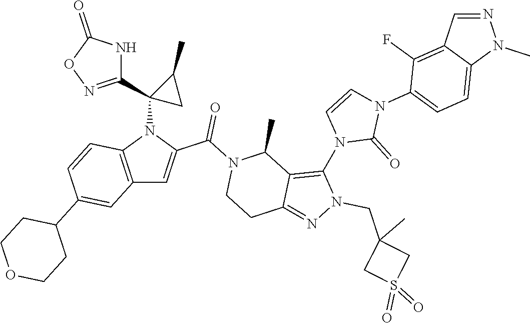 [SMILES: C[C@H]1c2c(nn(CC3(C)CS(=O)(=O)C3)c2-n2ccn(-c3ccc4c(cnn4C)c3F)c2=O)CCN1C(=O)c1cc2cc(C3CCOCC3)ccc2n1[C@@]1(c2noc(=O)[nH]2)C[C@@H]1C] | 3-((1S,2S)-1-(2-((S)-3-(3-(4- fluoro-1-methyl-1H-indazol-5- yl)-2-oxo-2,3-dihydro-1H- imidazol-1-yl)-4-methyl-2-((3- methyl-1,1-dioxidothietan-3- yl)methyl)-4,5,6,7-tetrahydro-2H- pyrazolo[4,3-c]pyridine-5- carbonyl)-5-(tetrahydro-2H- pyran-4-yl)-1H-indol-1-yl)-2- methylcyclopropyl)-1,2,4- oxadiazol-5(4H)-one | 865.5 |
| 12 | 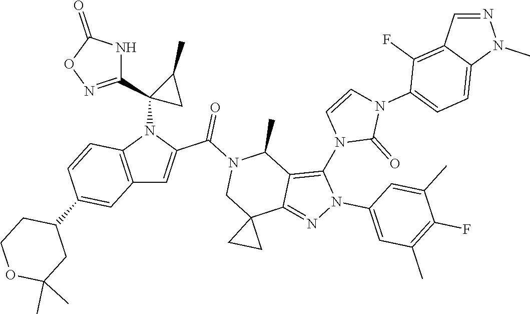 | 3-((1S,2S)-1-(5-((S)-2,2- dimethyltetrahydro-2H-pyran-4- yl)-2-((S)-3′-(3-(4-fluoro-1- methyl-1H-indazol-5-yl)-2-oxo- 2,3-dihydro-1H-imidazol-1-yl)-2′- (4-fluoro-3,5-dimethylphenyl)-4'- methyl-2′,4′,5′,6′- tetrahydrospiro[cyclopropane- 1,7′-pyrazolo[4,3-c]pyridine]-5′- carbonyl)-1H-indol-1-yl)-2- methylcyclopropyl)-1,2,4- oxadiazol-5(4H)-one | 909.6 |
| 13 | 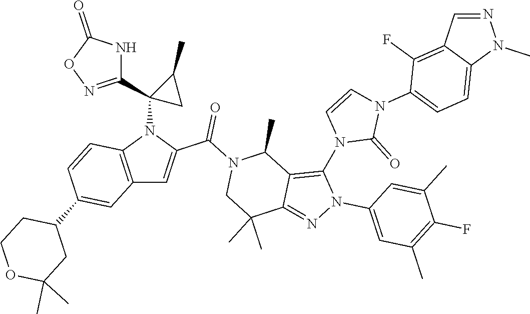 | 3-((1S,2S)-1-(5-((S)-2,2- dimethyltetrahydro-2H-pyran-4- yl)-2-((S)-3-(3-(4-fluoro-1- methyl-1H-indazol-5-yl)-2-oxo- 2,3-dihydro-1H-imidazol-1-yl)-2- (4-fluoro-3,5-dimethylphenyl)- 4,7,7-trimethyl-4,5,6,7- tetrahydro-2H-pyrazolo[4,3- c]pyridine-5-carbonyl)-1H-indol- 1-yl)-2-methylcyclopropyl)-1,2,4- oxadiazol-5(4H)-one | 911.8 |
| 14 | 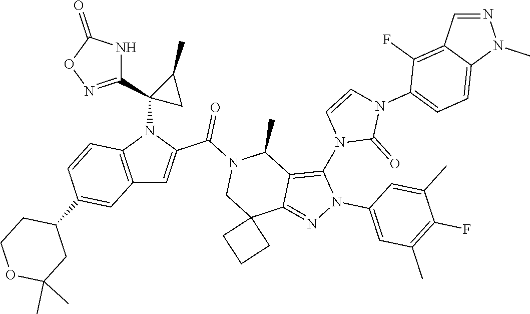 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)C[C@@H]2C)CC32CCC2)cc(C)c1F] | (4′S)-5′-(5-(2,2- dimethyltetrahydro-2H-pyran-4- yl)-1-((1S,2S)-2-methyl-1-(5- oxo-4,5-dihydro-1,2,4-oxadiazol- 3-yl)cyclopropyl)-1H-indole-2- carbonyl)-3′-(3-(4-fluoro-1- methyl-1H-indazol-5-yl)-2-oxo- 2,3-dihydro-1H-imidazol-1-yl)-2′- (4-fluoro-3,5-dimethylphenyl)-4′- methyl-2′,4′,5′,6′- tetrahydrospiro[cyclobutane-1,7′- pyrazolo[4,3-c]pyridin]-l′-ium | 923.0 |
| 15 | 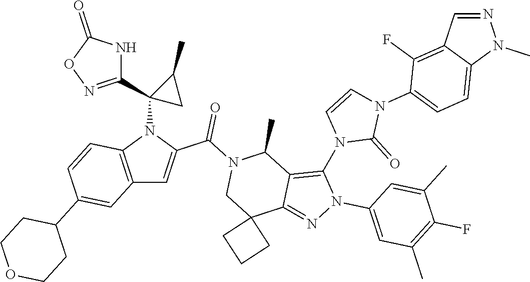 [SMILES: Cc1cc(-n2nc3c(c2-n2ccn(-c4ccc5c(cnn5C)c4F)c2=O)[C@H](C)N(C(=O)c2cc4cc(C5CCOCC5)ccc4n2[C@@]2(c4noc(=O)[nH]4)C[C@@H]2C)CC32CCC2)cc(C)c1F] | 3-((1S,2S)-1-(2-((S)-3′-(3-(4- fluoro-1-methyl-1H-indazol-5- yl)-2-oxo-2,3-dihydro-1H- imidazol-1-yl)-2′-(4-fluoro-3,5- dimethylphenyl)-4′-methyl- 2′,4′,5′,6′- tetrahydrospiro[cyclobutane-1,7′- pyrazolo[4,3-c]pyridine]-5′- carbonyl)-5-(tetrahydro-2H- pyran-4-yl)-1H-indol-1-yl)-2- methylcyclopropyl)-1,2,4- oxadiazol-5(4H)-one | 895.6 |
| 16 | 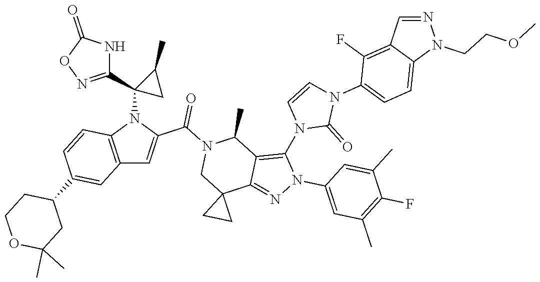 [SMILES: COCCn1ncc2c(F)c(-n3ccn(-c4c5c(nn4-c4cc(C)c(F)c(C)c4)C4(CC4)CN(C(=O)c4cc6cc([C@H]7CCOC(C)(C)C7)ccc6n4[C@@]4(c6noc(=O)[nH]6)C[C@@H]4C)[C@H]5C)c3=O)ccc21] | 3-((1S,2S)-1-(5-(2,2- dimethyltetrahydro-2H-pyran-4- yl)-2-((S)-3′-(3-(4-fluoro-1-(2- methoxyethyl)-1H-indazol-5-yl)- 2-oxo-2,3-dihydro-1H-imidazol- 1-yl)-2′-(4-fluoro-3,5- dimethylphenyl)-4′-methyl- 2′,4′,5′,6′- etrahydrospiro[cyclopropane-1,7′- pyrazolo[4,3-c]pyridine]-5′- carbonyl)-1H-indol-1-yl)-2- methylcyclopropyl)-1,2,4- oxadiazol-5(4H)-one | 953.9 |
| 17 | 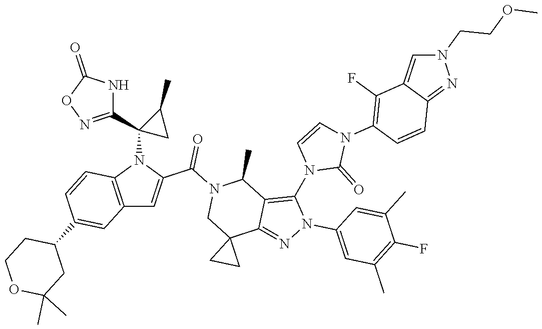 [SMILES: COCCn1cc2c(F)c(-n3ccn(-c4c5c(nn4-c4cc(C)c(F)c(C)c4)C4(CC4)CN(C(=O)c4cc6cc([C@H]7CCOC(C)(C)C7)ccc6n4[C@@]4(c6noc(=O)[nH]6)C[C@@H]4C)[C@H]5C)c3=O)ccc2n1] | 3-((1S,2S)-1-(5-(2,2- dimethyltetrahydro-2H-pyran-4- yl)-2-((S)-3′-(3-(4-fluoro-2-(2- methoxyethyl)-2H-indazol-5-yl)- 2-oxo-2,3-dihydro-1H-imidazol- 1-yl)-2′-(4-fluoro-3,5- dimethylphenyl)-4′-methyl- 2′,4′,5′,6′- tetrahydrospiro[cyclopropane- 1,7′-pyrazolo[4,3-c]pyridine]-5′- carbonyl)-1H-indol-1-yl)-2- methylcyclopropyl)-1,2,4- oxadiazol-5(4H)-one | 953.4 |
| 18 | 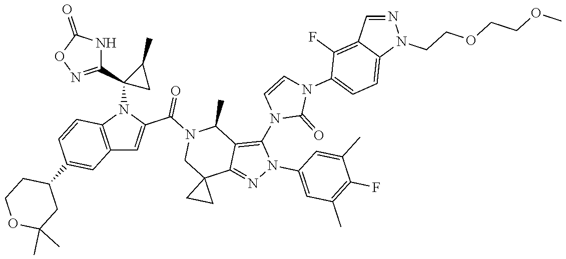 [SMILES: COCCOCCn1ncc2c(F)c(-n3ccn(-c4c5c(nn4-c4cc(C)c(F)c(C)c4)C4(CC4)CN(C(=O)c4cc6cc([C@H]7CCOC(C)(C)C7)ccc6n4[C@@]4(c6noc(=O)[nH]6)C[C@@H]4C)[C@H]5C)c3=O)ccc21] | 3-((1S,2S)-1-(5-(2,2- dimethyltetrahydro-2H-pyran-4- yl)-2-((S)-3′-(3-(4-fluoro-1-(2-(2- methoxyethoxy)ethyl)-1H- indazol-5-yl)-2-oxo-2,3-dihydro- 1H-imidazol-1-yl)-2′-(4-fluoro- 3,5-dimethylphenyl)-4'-methyl- 2′,4′,5′,6′- tetrahydrospiro[cyclopropane- 1,7′-pyrazolo[4,3-c]pyridine]-5′- carbonyl)-1H-indol-1-yl)-2- methylcyclopropyl)-1,2,4- oxadiazol-5(4H)-one | 997.8 |
| 19 | 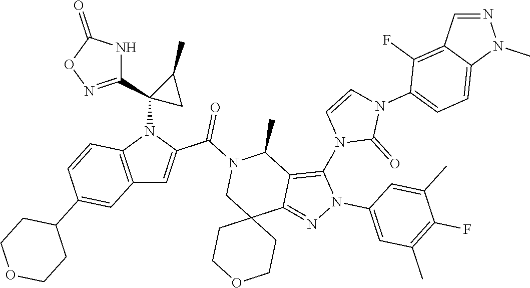 [SMILES: Cc1cc(-n2nc3c(c2-n2ccn(-c4ccc5c(cnn5C)c4F)c2=O)[C@H](C)N(C(=O)c2cc4cc(C5CCOCC5)ccc4n2[C@@]2(c4noc(=O)[nH]4)C[C@@H]2C)CC32CCOCC2)cc(C)c1F] | 3-((1S,2S)-1-(2-((S)-3′-(3-(4- fluoro-1-methyl-1H-indazol-5- yl)-2-oxo-2,3-dihydro-1H- imidazol-1-yl)-2′-(4-fluoro-3,5- dimethylphenyl)-4′-methyl- 2,2′,3,4′,5,5′,6,6′- octahydrospiro[pyran-4,7′- pyrazolo[4,3-c]pyridine]-5′- carbonyl)-5-(tetrahydro-2H- pyran-4-yl)-1H-indol-1-yl)-2- methylcyclopropyl)-1,2,4- oxadiazol-5(4H)-one | 925.6 |
| 170 | 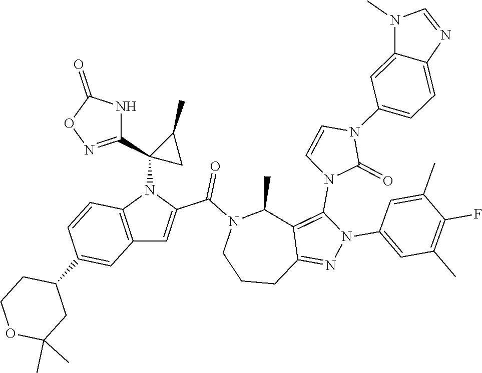 [SMILES: Cc1cc(-n2nc3c(c2-n2ccn(-c4ccc5ncn(C)c5c4)c2=O)[C@H](C)N(C(=O)c2cc4cc([C@H]5CCOC(C)(C)C5)ccc4n2[C@@]2(c4noc(=O)[nH]4)C[C@@H]2C)CCC3)cc(C)c1F] | 3-[(1S,2S)-1-[5-[(4S)-2,2- dimethyltetrahydropyran-4-yl]-2- [(4S)-2-(4-fluoro-3,5-dimethyl- phenyl)-4-methyl-3-[3-(3- methylbenzimidazol-1-ium-5-yl)- 2-oxo-imidazol-1-yl]-4,6,7,8- tetrahydropyrazolo[4,3-c]azepine- 5-carbonyl]indol-1-yl]-2-methyl- cyclopropyl]-4H-1,2,4-oxadiazol- 5-one formate | 879.4 |
| 171 | 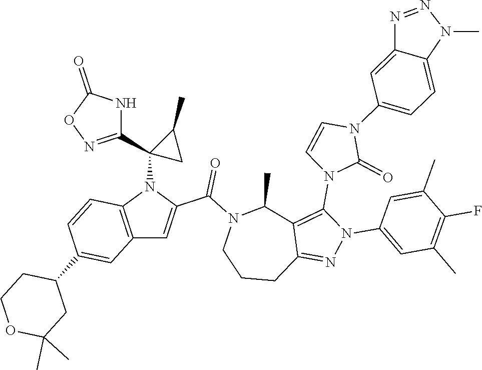 [SMILES: Cc1cc(-n2nc3c(c2-n2ccn(-c4ccc5c(c4)nnn5C)c2=O)[C@H](C)N(C(=O)c2cc4cc([C@H]5CCOC(C)(C)C5)ccc4n2[C@@]2(c4noc(=O)[nH]4)C[C@@H]2C)CCC3)cc(C)c1F] | 3-[(1S,2S)-1-[5-[(4S)-2,2- dimethyltetrahydropyran-4-yl]-2- [(4S)-2-(4-fluoro-3,5-dimethyl- phenyl)-4-methyl-3-[3-(1- methylbenzotriazol-5-yl)-2-oxo- imidazol-1-yl]-4,6,7,8- tetrahydropyrazolo[4,3-c]azepin- 1-ium-5-carbonyl]indol-1-yl]-2- methyl-cyclopropyl]-4H-1,2,4- oxadiazol-5-one formate | 880.4 |
| 172 | 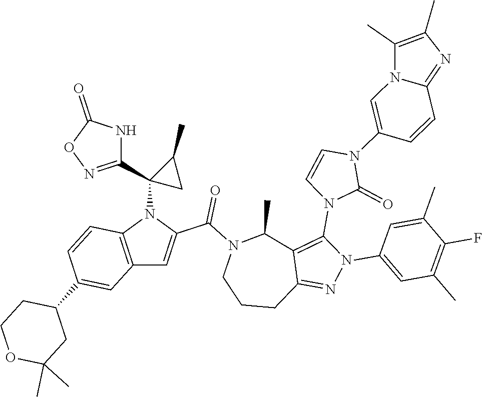 [SMILES: Cc1cc(-n2nc3c(c2-n2ccn(-c4ccc5nc(C)c(C)n5c4)c2=O)[C@H](C)N(C(=O)c2cc4cc([C@H]5CCOC(C)(C)C5)ccc4n2[C@@]2(c4noc(=O)[nH]4)C[C@@H]2C)CCC3)cc(C)c1F] | 3-[(1S,2S)-1-[2-[(4S)-3-[3-(2,3- dimethylimidazo[1,2-a]pyridin-1- ium-6-yl)-2-oxo-imidazol-1-yl]- 2-(4-fluoro-3,5-dimethyl-phenyl)- 4-methyl-4,6,7,8- tetrahydropyrazolo[4,3-c]azepine- 5-carbonyl]-5-[(4S)-2,2- dimethyltetrahydropyran-4- yl]indol-1-yl]-2-methyl- cyclopropyl]-4H-1,2,4-oxadiazol- 5-one formate | 893.8 |
| 173 | 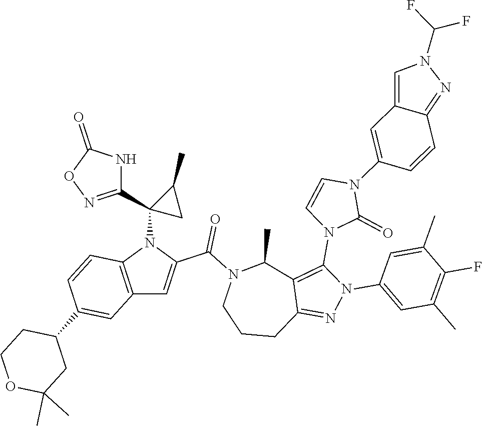 [SMILES: Cc1cc(-n2nc3c(c2-n2ccn(-c4ccc5nn(C(F)F)cc5c4)c2=O)[C@H](C)N(C(=O)c2cc4cc([C@H]5CCOC(C)(C)C5)ccc4n2[C@@]2(c4noc(=O)[nH]4)C[C@@H]2C)CCC3)cc(C)c1F] | 3-[(1S,2S)-1-[2-[(4S)-3-[3-[2- (difluoromethyl)indazol-5-yl]-2- oxo-imidazol-1-yl]-2-(4-fluoro- 3,5-dimethyl-phenyl)-4-methyl- 4,6,7,8-tetrahydropyrazolo[4,3- c]azepin-1-ium-5-carbonyl]-5- [(4S)-2,2- dimethyltetrahydropyran-4- yl]indol-1-yl]-2-methyl- cyclopropyl]-4H-1,2,4-oxadiazol- 5-one formate | 915.4 |
| 174 | 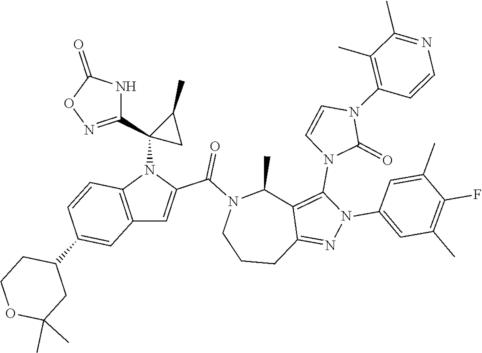 | 3-[(1S,2S)-1-[2-[(4S)-3-[3-(2,3- dimethylpyridin-1-ium-4-yl)-2- oxo-imidazol-1-yl]-2-(4-fluoro- 3,5-dimethyl-phenyl)-4-methyl- 4,6,7,8-tetrahydropyrazolo[4,3- c]azepine-5-carbonyl]-5-[(4S)- 2,2-dimethyltetrahydropyran-4- yl]indol-1-yl]-2-methyl- cyclopropyl]-4H-1,2,4-oxadiazol- 5-one formate | 854.4 |
| 175 | 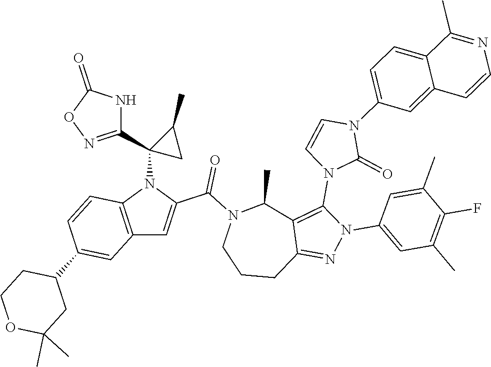 | 3-[(1S,2S)-1-[5-[(4S)-2,2- dimethyltetrahydropyran-4-yl]-2- [(4S)-2-(4-fluoro-3,5-dimethyl- phenyl)-4-methyl-3-[3-(1- methylisoquinolin-2-ium-6-yl)-2- oxo-imidazol-1-yl]-4,6,7,8- tetrahydropyrazolo[4,3-c]azepine- 5-carbonyl]indol-1-yl]-2-methyl- cyclopropyl]-4H-1,2,4-oxadiazol- 5-one formate | 890.4 |
| 176 | 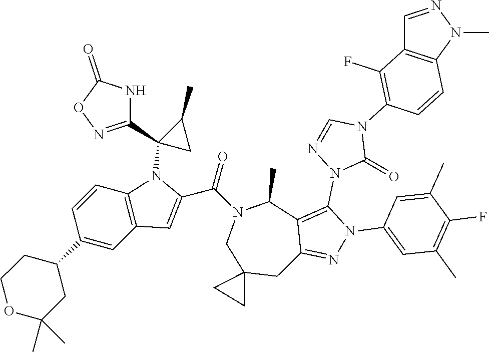 [SMILES: Cc1cc(-n2nc3c(c2-n2ncn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)C[C@@H]2C)CC2(CC2)C3)cc(C)c1F] | 3-[(1S,2S)-1-[5-[(4S)-2,2- dimethyltetrahydropyran-4-yl]-2- [(4S)-2-(4-fluoro-3,5-dimethyl- phenyl)-3-[3-(4-fluoro-1-methyl- indazol-5-yl)-2-oxo-imidazol-1- yl]-4-methyl-spiro[6,8-dihydro- 4H-pyrazolo[4,3-c]azepin-1-ium- 7,1′-cyclopropane]-5- carbonyl]indol-1-yl]-2-methyl- cyclopropyl]-4H-1,2,4-oxadiazol- 5-one formate | 923.4 |
| 177 | 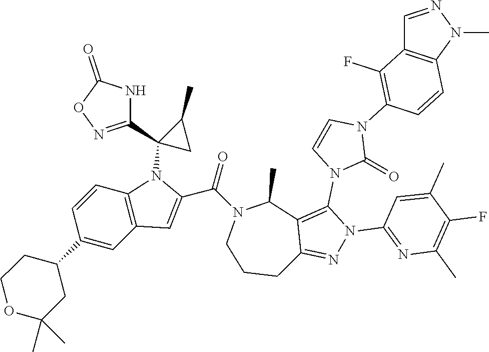 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)C[C@@H]2C)CCC3)nc(C)c1F] | 3-[(1S,2S)-1-[5-[(4S)-2,2- dimethyltetrahydropyran-4-yl]-2- [(4S)-2-(5-fluoro-4,6-dimethyl- pyridin-1-ium-2-yl)-3-[3-(4- fluoro-1-methyl-indazol-5-yl)-2- oxo-imidazol-1-yl]-4-methyl- 4,6,7,8-tetrahydropyrazolo[4,3- c]azepine-5-carbonyl]indol-1-yl]- 2-methyl-cyclopropyl]-4H-1,2,4- oxadiazol-5-one formate | 899.2 |
| 178 | 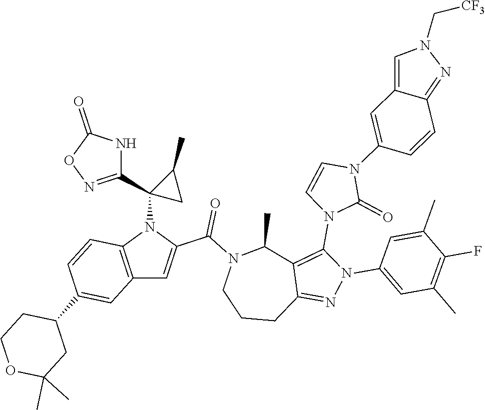 [SMILES: Cc1cc(-n2nc3c(c2-n2ccn(-c4ccc5nn(CC(F)(F)F)cc5c4)c2=O)[C@H](C)N(C(=O)c2cc4cc([C@H]5CCOC(C)(C)C5)ccc4n2[C@@]2(c4noc(=O)[nH]4)C[C@@H]2C)CCC3)cc(C)c1F] | 3-[(1S,2S)-1-[5-[(4S)-2,2- dimethyltetrahydropyran-4-yl]-2- [(4S)-2-(4-fluoro-3,5-dimethyl- phenyl)-4-methyl-3-[2-oxo-3-[2- (2,2,2-trifluoroethyl)indazol-5- yl]imidazol-1-yl]-4,6,7,8- tetrahydropyrazolo[4,3-c]azepin- 1-ium-5-carbonyl]indol-1-yl]-2- methyl-cyclopropyl]-4H-1,2,4- oxadiazol-5-one formate | 947.6 |
| 179 | 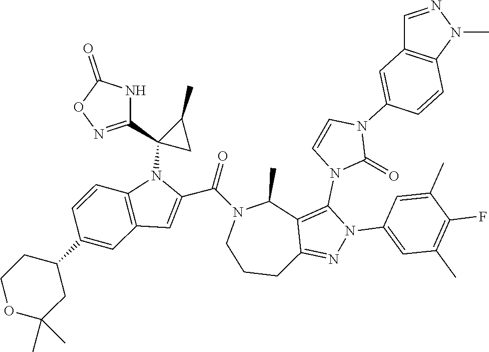 [SMILES: Cc1cc(-n2nc3c(c2-n2ccn(-c4ccc5c(cnn5C)c4)c2=O)[C@H](C)N(C(=O)c2cc4cc([C@H]5CCOC(C)(C)C5)ccc4n2[C@@]2(c4noc(=O)[nH]4)C[C@@H]2C)CCC3)cc(C)c1F] | 3-[(1S,2S)-1-[5-[(4S)-2,2- dimethyltetrahydropyran-4-yl]-2- [(4S)-2-(4-fluoro-3,5-dimethyl- phenyl)-4-methyl-3-[3-(1- methylindazol-5-yl)-2-oxo- imidazol-1-yl]-4,6,7,8- tetrahydropyrazolo[4,3-c]azepin- 1-ium-5-carbonyl]indol-1-yl]-2- methyl-cyclopropyl]-4H-1,2,4- oxadiazol-5-one formate | 879.4 |
| 180 | 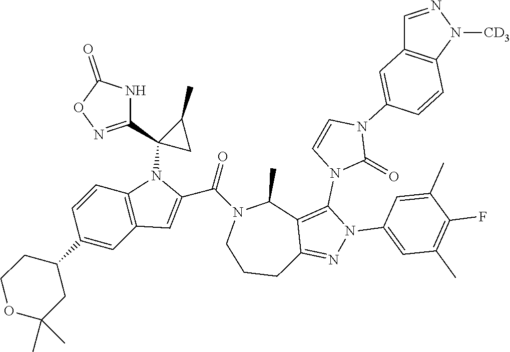 [SMILES: [2H]C([2H])([2H])n1ncc2cc(-n3ccn(-c4c5c(nn4-c4cc(C)c(F)c(C)c4)CCCN(C(=O)c4cc6cc([C@H]7CCOC(C)(C)C7)ccc6n4[C@@]4(c6noc(=O)[nH]6)C[C@@H]4C)[C@H]5C)c3=O)ccc21] | 3-[(1S,2S)-1-[5-[(4S)-2,2- dimethyltetrahydropyran-4-yl]-2- [(4S)-2-(4-fluoro-3,5-dimethyl- phenyl)-4-methyl-3-[2-oxo-3-[1- (trideuteriomethyl)indazol-5- yl]imidazol-1-yl]-4,6,7,8- tetrahydropyrazolo[4,3-c]azepin- 1-ium-5-carbonyl]indol-1-yl]-2- methyl-cyclopropyl]-4H-1,2,4- oxadiazol-5-one formate | 882.6 |
| 181 | 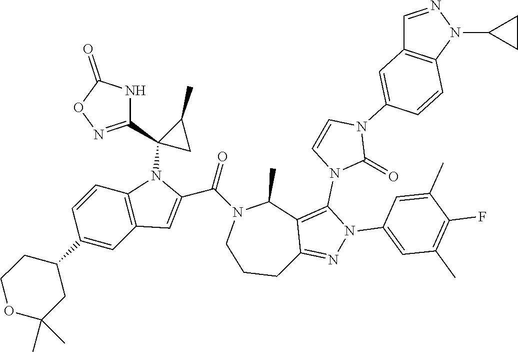 [SMILES: Cc1cc(-n2nc3c(c2-n2ccn(-c4ccc5c(cnn5C5CC5)c4)c2=O)[C@H](C)N(C(=O)c2cc4cc([C@H]5CCOC(C)(C)C5)ccc4n2[C@@]2(c4noc(=O)[nH]4)C[C@@H]2C)CCC3)cc(C)c1F] | 3-[(1S,2S)-1-[2-[(4S)-3-[3-(1- cyclopropylindazol-5-yl)-2-oxo- imidazol-1-yl]-2-(4-fluoro-3,5- dimethyl-phenyl)-4-methyl- 4,6,7,8-tetrahydropyrazolo[4,3- c]azepin-1-ium-5-carbonyl]-5- [(4S)-2,2- dimethyltetrahydropyran-4- yl]indol-1-yl]-2-methyl- cyclopropyl]-4H-1,2,4-oxadiazol- 5-one formate | 905.6 |
| 182 | 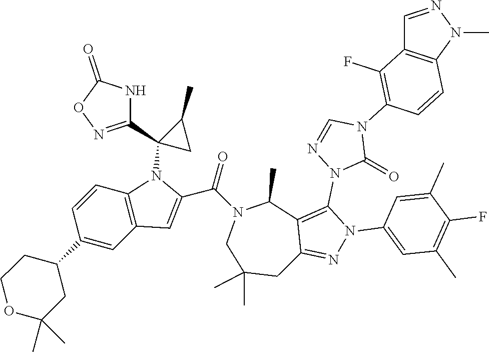 [SMILES: Cc1cc(-n2nc3c(c2-n2ncn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)C[C@@H]2C)CC(C)(C)C3)cc(C)c1F] | 3-[(1S,2S)-1-[5-[(4S)-2,2- dimethyltetrahydropyran-4-yl]-2- [(4S)-2-(4-fluoro-3,5-dimethyl- phenyl)-3-[3-(4-fluoro-1-methyl- indazol-5-yl)-2-oxo-imidazol-1- yl]-4,7,7-trimethyl-6,8-dihydro- 4H-pyrazolo[4,3-c]azepine-5- carbonyl]indol-1-yl]-2-methyl- cyclopropyl]-4H-1,2,4-oxadiazol- 5-one | 925.4 |
| 183 | 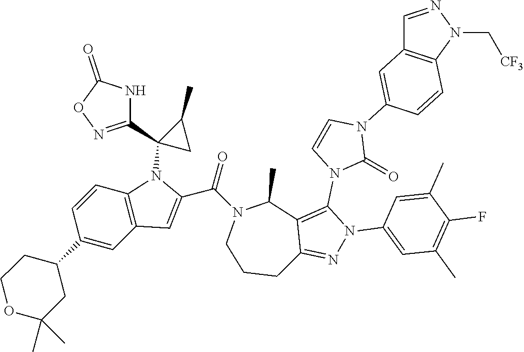 [SMILES: Cc1cc(-n2nc3c(c2-n2ccn(-c4ccc5c(cnn5CC(F)(F)F)c4)c2=O)[C@H](C)N(C(=O)c2cc4cc([C@H]5CCOC(C)(C)C5)ccc4n2[C@@]2(c4noc(=O)[nH]4)C[C@@H]2C)CCC3)cc(C)c1F] | 3-[(1S,2S)-1-[5-[(4S)-2,2- dimethyltetrahydropyran-4-yl]-2- [(4S)-2-(4-fluoro-3,5-dimethyl- phenyl)-4-methyl-3-[2-oxo-3-[1- (2,2,2-trifluoroethyl)indazol-5- yl]imidazol-1-yl]-4,6,7,8- tetrahydropyrazolo[4,3-c]azepin- 1-ium-5-carbonyl]indol-1-yl]-2- methyl-cyclopropyl]-4H-1,2,4- oxadiazol-5-one formate | 947.6 |
| 184 | 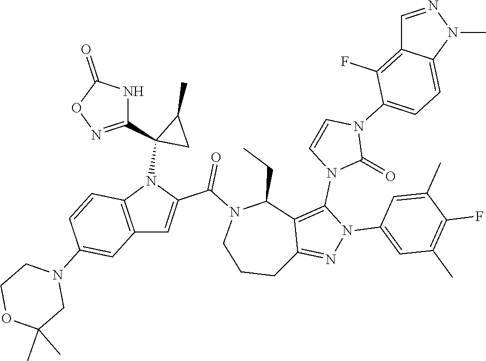 [SMILES: CC[C@H]1c2c(nn(-c3cc(C)c(F)c(C)c3)c2-n2ccn(-c3ccc4c(cnn4C)c3F)c2=O)CCCN1C(=O)c1cc2cc(N3CCOC(C)(C)C3)ccc2n1[C@@]1(c2noc(=O)[nH]2)C[C@@H]1C] | 3-((1S,2S)-1-(5-(2,2- dimethylmorpholino)-2-((4S)-4- ethyl-3-(3-(4-fluoro-1-methyl- 1H-indazol-5-yl)-2-oxo-2,3- dihydro-1H-imidazol-1-yl)-2-(4- fluoro-3,5-dimethylphenyl)- 2,4,5,6,7,8- hexahydropyrazolo[4,3- c]azepine-5-carbonyl)-1H-indol- 1-yl)-2-methylcyclopropyl)-1,2,4- oxadiazol-5(4H)-one | 911.4 |
| 185 | 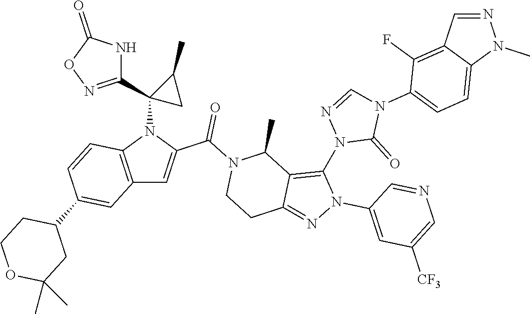 | 3-((1S,2S)-1-(5-((S)-2,2- dimethyltetrahydro-2H-pyran-4- yl)-2-((4S)-3-(3-(4-fluoro-1- methyl-1H-indazol-5-yl)-2-oxo- 2,3-dihydro-1H-imidazol-1-yl)-4- methyl-2-(5- (trifluoromethyl)pyridin-3-yl)- 4,5,6,7-tetrahydro-2H- pyrazolo[4,3-c]pyridine-5- carbonyl)-1H-indol-1-yl)-2- methylcyclopropyl)-1,2,4- oxadiazol-5(4H)-one | 906.6 |
| 186 | 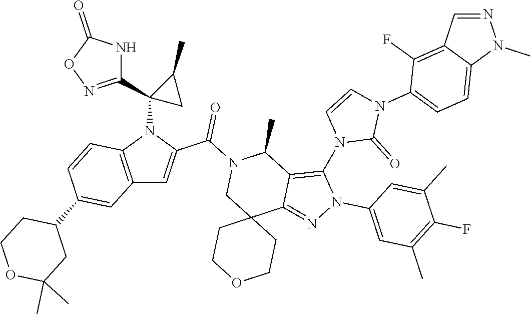 | 3-((1S,2S)-1-(5-((S)-2,2- dimethyltetrahydro-2H-pyran-4- yl)-2-((4′S)-3′-(3-(4-fluoro-1- methyl-1H-indazol-5-yl)-2-oxo- 2,3-dihydro-1H-imidazol-1-yl)-2′- (4-fluoro-3,5-dimethylphenyl)-4′- methyl-2,2′,3,4′,5,5′,6,6′- octahydrospiro[pyran-4,7′- pyrazolo[4,3-c]pyridine]-5′- carbonyl)-1H-indol-1-yl)-2- methylcyclopropyl)-1,2,4- oxadiazol-5(4H)-one | 953.8 |
| 330 | 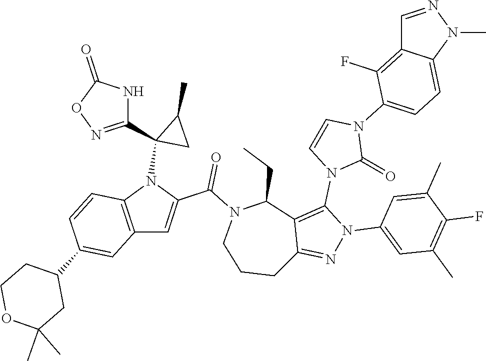 | 3-((1S,2S)-1-(5-((S)-2,2- dimethyltetrahydro-2H-pyran-4- yl)-2-((4S)-4-ethyl-3-(3-(4- fluoro-1-methyl-1H-indazol-5- yl)-2-oxo-2,3-dihydro-1H- imidazol-1-yl)-2-(4-fluoro-3,5- dimethylphenyl)-2,4,5,6,7,8- hexahydropyrazolo[4,3- c]azepine-5-carbonyl)-1H-indol- 1-yl)-2-methylcyclopropyl)-1,2,4- oxadiazol-5(4H)-one | 911.0 |
| 336 | 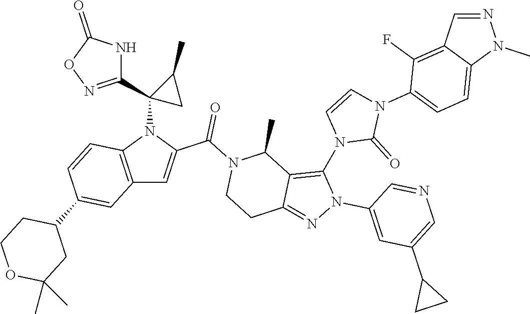 | 3-((1S,2S)-1-(2-((4S)-2-(5- cyclopropylpyridin-3-yl)-3-(3-(4- fluoro-1-methyl-1H-indazol-5- yl)-2-oxo-2,3-dihydro-1H- imidazol-1-yl)-4-methyl-4,5,6,7- tetrahydro-2H-pyrazolo[4,3- c]pyridine-5-carbonyl)-5-((S)-2,2- dimethyltetrahydro-2H-pyran-4- yl)-1H-indol-1-yl)-2- methylcyclopropyl)-1,2,4- oxadiazol-5(4H)-one | 878.4 |

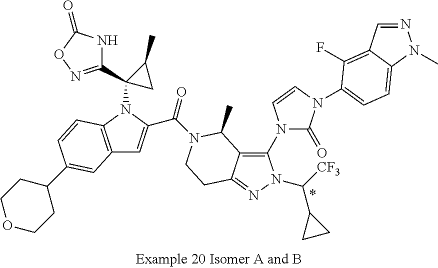 [SMILES: C[C@H]1c2c(nn(C(C3CC3)C(F)(F)F)c2-n2ccn(-c3ccc4c(cnn4C)c3F)c2=O)CCN1C(=O)c1cc2cc(C3CCOCC3)ccc2n1[C@@]1(c2noc(=O)[nH]2)C[C@@H]1C]

### 3-((1S,2S)-1-(2-((S)-2-((S)-1-cyclopropyl-2,2,2-trifluoroethyl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one and 3-((1S,2S)-1-(2-((S)-2-((R)-1-cyclopropyl-2,2,2-trifluoroethyl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one (scheme 1)

### Step 1: tert-butyl (4S)-2-(1-cyclopropyl-2,2,2-trifluoroethyl)-3-(3-(2,2-dimethoxyethyl)ureido)-4-methyl-2,4,6,7-tetrahydro-5H-pyrazolo[4,3-c]pyridine-5-carboxylate

This step was performed in a similar fashion as described for example 1 to provide the title compound. MS (ESI) m/z: 506.2 [M+H]+

### Step 2: tert-butyl (4S)-2-(1-cyclopropyl-2,2,2-trifluoroethyl)-4-methyl-3-(2-oxo-2,3-dihydro-1H-imidazol-1-yl)-2,4,6,7-tetrahydro-5H-pyrazolo[4,3-c]pyridine-5-carboxylate

To a solution of tert-butyl (4S)-2-(1-cyclopropyl-2,2,2-trifluoroethyl)-3-(3-(2,2-dimethoxyethyl)ureido)-4-methyl-2,4,6,7-tetrahydro-5H-pyrazolo[4,3-c]pyridine-5-carboxylate (440 mg, 0.87 mmol) in THF (7.5 mL) was added methanesulfonic acid (67 mg, 0.70 mmol). The reaction mixture was stirred at 60° C. for 3 h. Then, the reaction mixture was used in the next step without further purification. LCMS (ESI) m/z: 342.2 [M+H]+. The crude mixture from the previous reaction was cooled in an ice-water bath and the pH was adjusted to ˜9 with potassium phosphate tribasic (2.16 mL, 4.32 mmol), then di-tert-butyl dicarbonate (75 mg, 0.35 mmol) was added to the reaction mixture. The mixture was stirred at 10° C. for 1 h. Then, the reaction mixture was poured into water (40 mL) and extracted with ethyl acetate (3×30 mL). The combined organic layers were washed with brine (3×20 mL), dried over Na₂SO₄, filtered, and concentrated under reduced pressure to provide the title compound. MS (ESI) m/z: 442.1 [M+H]+.

### Step 3: tert-butyl (S)-2-((R)-1-cyclopropyl-2,2,2-trifluoroethyl)-3-(3-(4-fluoro-1-methyl-1H-indazol-5-yl)-2-oxo-2,3-dihydro-1H-imidazol-1-yl)-4-methyl-2,4,6,7-tetrahydro-5H-pyrazolo[4,3-c]pyridine-5-carboxylate and tert-butyl (S)-2-((S)-1-cyclopropyl-2,2,2-trifluoroethyl)-3-(3-(4-fluoro-1-methyl-1H-indazol-5-yl)-2-oxo-2,3-dihydro-1H-imidazol-1-yll-4-methyl-2,4,6,7-tetrahydro-5H-pyrazolo[4,3-c]pyridine-5-carboxylate

A mixture of tert-butyl (4S)-2-(1-cyclopropyl-2,2,2-trifluoroethyl)-4-methyl-3-(2-oxo-2,3-dihydro-1H-imidazol-1-yl)-2,4,6,7-tetrahydro-5H-pyrazolo[4,3-c]pyridine-5-carboxylate (700 mg, 1.586 mmol), 5-bromo-4-fluoro-1-methyl-1H-indazole (545 mg, 2.378 mmol), copper(I) iodide (151 mg, 0.793 mmol), K₃PO₄ (1.68 g, 7.93 mmol) and (1R,2R)—N1,N2-dimethylcyclohexane-1,2-diamine (90 mg, 0.634 mmol) in dioxane (7.0 mL) under an atmosphere of N₂ was stirred at 100° C. for 16 h. Then, the mixture was cooled to rt and quenched by the addition of water (20 mL) and extracted with ethyl acetate (3×20 mL). The combined organic layers were washed with brine (3×20 mL), dried over Na₂SO₄, filtered, and concentrated under reduced pressure. The crude residue was purified by silica gel chromatography (0% to 55% EtOAc/Pet.ether. The enantiomers of the product were separated by SFC (Daicel CHIRALPAK® AD(250 mm*30 mm, 10 μm); condition CO₂—EtOH(0.1%NH3H2O); B %: 15% isocratic elution mode) to obtain the enantiomer title compounds. Data for the faster eluting isomer A: ¹H NMR of (400 MHz, methanol-d4) δ=8.18 (s, 1H), 7.60-7.46 (m, 2H), 6.98 (d, J=2.9 Hz, 1H), 6.83 (br s, 1H), 5.24 (br dd, J=1.3, 4.0 Hz, 1H), 4.41-4.28 (m, 1H), 4.13 (s, 3H), 4.06-3.96 (m, 1H), 3.23-3.02 (m, 1H), 2.83-2.64 (m, 2H), 1.88-1.74 (m, 1H), 1.51 (s, 9H), 1.22 (d, J=6.7 Hz, 3H), 0.87 (br t, J=7.9 Hz, 1H), 0.72-0.52 (m, 3H). MS (ESI) m/z: 590.2 [M+H]+. Data for the slower eluting isomer B: ¹H NMR of B-001(400 MHz, methanol-d4) δ=8.18 (s, 1H), 7.62-7.47 (m, 2H), 6.97 (d, J=3.1 Hz, 1H), 6.73 (br s, 1H), 5.25-5.12 (m, 1H), 4.41-4.27 (m, 1H), 4.13 (s, 3H), 3.99-3.88 (m, 1H), 3.23-3.10 (m, 1H), 2.82-2.65 (m, 2H), 1.80-1.68 (m, 1H), 1.49 (s, 9H), 1.35-1.28 (m, 3H), 0.90-0.79 (m, 1H), 0.73-0.66 (m, 1H), 0.64-0.55 (m, 1H), 0.43 (br s, 1H). MS (ESI) m/z: 590.2 [M+H]+.

### Steps 4 and 5: 3-((1S,2S)-1-(2-((S)-2-((S)-1-cyclopropyl-2,2,2-trifluoroethyl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one and 3-((1S,2S)-1-(2-((S)-2-((R)-1-cyclopropyl-2,2,2-trifluoroethyl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one

These steps were performed in a similar fashion as described for example 1 for each isomer separately to provide the title compounds as TFA salts. Data for isomer A: ¹H NMR (400 MHz, DMSO-d6) δ=11.81-11.69 (m, 1H), 11.92-11.61 (m, 1H), 11.83-11.60 (m, 1H), 8.38-8.18 (m, 1H), 7.77-7.34 (m, 4H), 7.30-7.14 (m, 2H), 6.95 (br s, 2H), 4.44-3.85 (m, 7H), 4.18-3.82 (m, 1H), 2.96-2.76 (m, 2H), 1.83-1.54 (m, 7H), 1.47-1.06 (m, 6H), 0.86-0.44 (m, 4H). MS (ESI) m/z: 855.3 [M+H]+. Data for isomer B: ¹H NMR (400 MHz, methanol-d4) δ=8.22-7.89 (m, 1H), 7.63-7.40 (m, 4H), 7.25-7.23 (m, 1H), 7.09-6.98 (m, 1H), 6.91-6.77 (m, 2H), 4.17-4.00 (m, 7H), 3.64-3.53 (m, 3H), 3.00-2.86 (m, 2H), 1.85-1.75 (m, 6H), 1.63 (br d, J=6.7 Hz, 2H), 1.58-1.50 (m, 3H), 1.22 (br d, J=5.4 Hz, 2H), 1.03-0.96 (m, 1H), 0.94-0.79 (m, 1H), 0.72 (br s, 2H). MS (ESI) m/z: 855.2 [M+H]+.

The following examples in table 1b were prepared according to scheme 1 using the procedures outlined in the synthesis of example 20. In cases wherein isomers of the examples were separated via chiral chromatography, conditions are included, and the faster eluting isomer is listed first.

TABLE 1b

| | | | MS |
|---|---|---|---|
| | | | (M + |
| Ex. | Structure | Name | 1) |

| | | | |
|---|---|---|---|
| 21 Isomer A (Regis Whelk- O ® 1 RR, 250 × 30 (ID) mm; CO2: MeOH + 0.01% NH₄OH) | 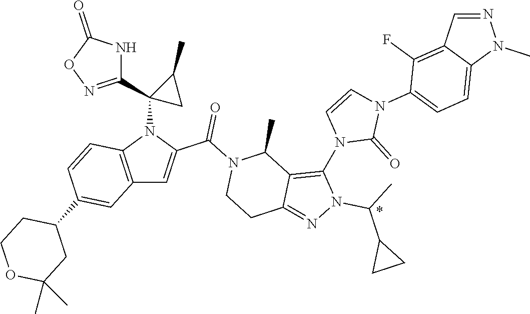 [SMILES: CC(C1CC1)n1nc2c(c1-n1ccn(-c3ccc4c(cnn4C)c3F)c1=O)[C@H](C)N(C(=O)c1cc3cc([C@H]4CCOC(C)(C)C4)ccc3n1[C@@]1(c3noc(=O)[nH]3)C[C@@H]1C)CC2] | 3-((1S,2S)-1-(2-((S)-2- ((R)-1-cyclopropylethyl)- 3-(3-(4-fluoro-1-methyl- 1H-indazol-5-yl)-2-oxo- 2,3-dihydro-1H-imidazol- 1-yl)-4-methyl-4,5,6,7- tetrahydro-2H- pyrazolo[4,3-c]pyridine-5- carbonyl)-5-((S)-2,2- dimethyltetrahydro-2H- pyran-4-yl)-1H-indol-1- yl)-2-methylcyclopropyl)- 1,2,4-oxadiazol-5(4H)-one or 3-((1S,2S)-1-(2-((S)-2- ((S)-1-cyclopropylethyl)- 3-(3-(4-fluoro-1-methyl- 1H-indazol-5-yl)-2-oxo- 2,3-dihydro-1H-imidazol- 1-yl)-4-methyl-4,5,6,7- tetrahydro-2H- | 829.8 |
| | | pyrazolo[4,3-c]pyridine-5- | |
| | | carbonyl)-5-((S)-2,2- | |
| | | dimethyltetrahydro-2H- | |
| | | pyran-4-yl)-1H-indol-1- | |
| | | yl)-2-methylcyclopropyl)- | |
| | | 1,2,4-oxadiazol-5(4H)-one | |
| 21 Isomer B (Regis Whelk- O ® 1 RR, 250 × 30 (ID) mm; CO2: MeOH + 0.01% NH₄OH) | 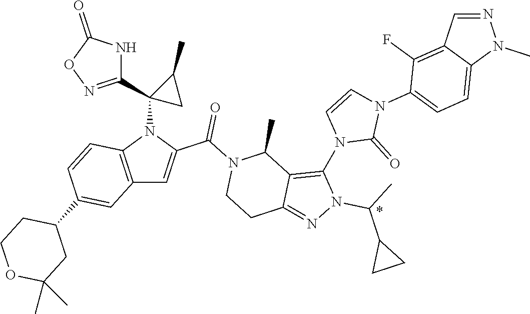 [SMILES: CC(C1CC1)n1nc2c(c1-n1ccn(-c3ccc4c(cnn4C)c3F)c1=O)[C@H](C)N(C(=O)c1cc3cc([C@H]4CCOC(C)(C)C4)ccc3n1[C@@]1(c3noc(=O)[nH]3)C[C@@H]1C)CC2] | 3-((1S,2S)-1-(2-((S)-2- ((R)-1-cyclopropylethyl)- 3-(3-(4-fluoro-1-methyl- 1H-indazol-5-yl)-2-oxo- 2,3-dihydro-1H-imidazol- 1-yl)-4-methyl-4,5,6,7- tetrahydro-2H- pyrazolo[4,3-c]pyridine-5- carbonyl)-5-((S)-2,2- dimethyltetrahydro-2H- pyran-4-yl)-1H-indol-1- yl)-2-methylcyclopropyl)- 1,2,4-oxadiazol-5(4H)-one or 3-((1S,2S)-1-(2-((S)-2- ((S)-1-cyclopropylethyl)- 3-(3-(4-fluoro-1-methyl- 1H-indazol-5-yl)-2-oxo- 2,3-dihydro-1H-imidazol- 1-yl)-4-methyl-4,5,6,7- tetrahydro-2H- | 829.8 |
| | | pyrazolo[4,3-c]pyridine-5- | |
| | | carbonyl)-5-((S)-2,2- | |
| | | dimethyltetrahydro-2H- | |
| | | pyran-4-yl)-1H-indol-1- | |
| | | yl)-2-methylcyclopropyl)- | |
| | | 1,2,4-oxadiazol-5(4H)-one | |
| 22 Isomer A (Regis Whelk- O ® 1 RR, 250 × 30 (ID) mm; CO₂: MeOH + 0.1% diethyl amine) | 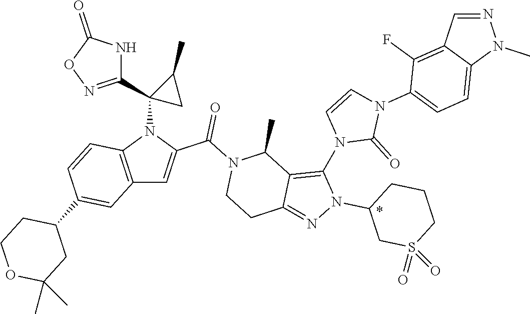 [SMILES: C[C@H]1c2c(nn(C3CCCS(=O)(=O)C3)c2-n2ccn(-c3ccc4c(cnn4C)c3F)c2=O)CCN1C(=O)c1cc2cc([C@H]3CCOC(C)(C)C3)ccc2n1[C@@]1(c2noc(=O)[nH]2)C[C@@H]1C] | 3-((1S,2S)-1-(5-((S)-2,2- dimethyltetrahydro-2H- pyran-4-yl)-2-((S)-2-((S)- 1,1-dioxidotetrahydro-2H- thiopyran-3-yl)-3-(3-(4- fluoro-1-methyl-1H- indazol-5-yl)-2-oxo-2,3- dihydro-1H-imidazol-1- yl)-4-methyl-4,5,6,7- tetrahydro-2H- pyrazolo[4,3-c]pyridine-5- carbonyl)-1H-indol-1-yl)- 2-methylcyclopropyl)- 1,2,4-oxadiazol-5(4H)-one or 3-((1S,2S)-1-(5-((S)- 2,2-dimethyltetrahydro- 2H-pyran-4-yl)-2-((S)-2- ((R)-1,1- dioxidotetrahydro-2H- thiopyran-3-yl)-3-(3-(4- | 893.6 |
| | | fluoro-1-methyl-1H- | |
| | | indazol-5-yl)-2-oxo-2,3- | |
| | | dihydro-1H-imidazol-1- | |
| | | yl)-4-methyl-4,5,6,7- | |
| | | tetrahydro-2H- | |
| | | pyrazolo[4,3-c]pyridine-5- | |
| | | carbonyl)-1H-indol-1-yl)- | |
| | | 2-methylcyclopropyl)- | |
| | | 1,2,4-oxadiazol-5(4H)-one | |
| 22 Isomer B (Regis Whelk- O ® 1 RR, 250 × 30 (ID) mm; CO₂: MeOH + 0.1% diethyl amine) | 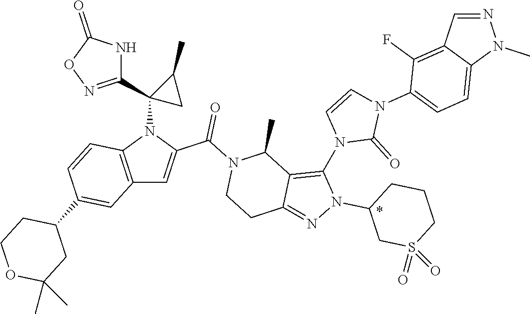 [SMILES: C[C@H]1c2c(nn(C3CCCS(=O)(=O)C3)c2-n2ccn(-c3ccc4c(cnn4C)c3F)c2=O)CCN1C(=O)c1cc2cc([C@H]3CCOC(C)(C)C3)ccc2n1[C@@]1(c2noc(=O)[nH]2)C[C@@H]1C] | 3-((1S,2S)-1-(5-((S)-2,2- dimethyltetrahydro-2H- pyran-4-yl)-2-((S)-2-((S)- 1,1-dioxidotetrahydro-2H- thiopyran-3-yl)-3-(3-(4- fluoro-1-methyl-1H- indazol-5-yl)-2-oxo-2,3- dihydro-1H-imidazol-1- yl)-4-methyl-4,5,6,7- tetrahydro-2H- pyrazolo[4,3-c]pyridine-5- carbonyl)-1H-indol-1-yl)- 2-methylcyclopropyl)- 1,2,4-oxadiazol-5(4H)-one or 3-((1S,2S)-1-(5-((S)- 2,2-dimethyltetrahydro- 2H-pyran-4-yl)-2-((S)-2- ((R)-1,1- dioxidotetrahydro-2H- | 893.6 |
| | | thiopyran-3-yl)-3-(3-(4- | |
| | | fluoro-1-methyl-1H- | |
| | | indazol-5-yl)-2-oxo-2,3- | |
| | | dihydro-1H-imidazol-1- | |
| | | yl)-4-methyl-4,5,6,7- | |
| | | tetrahydro-2H- | |
| | | pyrazolo[4,3-c]pyridine-5- | |
| | | carbonyl)-1H-indol-1-yl)- | |
| | | 2-methylcyclopropyl)- | |
| | | 1,2,4-oxadiazol-5(4H)-one | |
| 23 Isomer A (Daicel Chiralpak IG; CO₂:Et OH + 0.1% NH₃H₂O) | 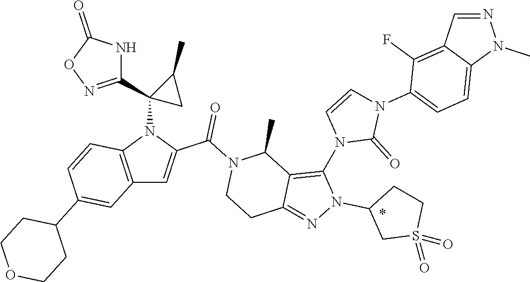 [SMILES: C[C@H]1c2c(nn(C3CCS(=O)(=O)C3)c2-n2ccn(-c3ccc4c(cnn4C)c3F)c2=O)CCN1C(=O)c1cc2cc(C3CCOCC3)ccc2n1[C@@]1(c2noc(=O)[nH]2)C[C@@H]1C] | 3-((1S,2S)-1-(2-((S)-2- ((S)-1,1- dioxidotetrahydrothiophen- 3-yl)-3-(3-(4-fluoro-1- methyl-1H-indazol-5-yl)- 2-oxo-2,3-dihydro-1H- imidazol-1-yl)-4-methyl- 4,5,6,7-tetrahydro-2H- pyrazolo[4,3-c]pyridine-5- carbonyl)-5-(tetrahydro- 2H-pyran-4-yl)-1H-indol- 1-yl)-2- methylcyclopropyl)-1,2,4- oxadiazol-5(4H)-one or 3- ((1S,2S)-1-(2-((S)-2-((R)- 1,1- dioxidotetrahydrothiophen- | 851.3 |
| | | 3-yl)-3-(3-(4-fluoro-1- | |
| | | methyl-1H-indazol-5-yl)- | |
| | | 2-oxo-2,3-dihydro-1H- | |
| | | imidazol-1-yl)-4-methyl- | |
| | | 4,5,6,7-tetrahydro-2H- | |
| | | pyrazolo[4,3-c]pyridine-5- | |
| | | carbonyl)-5-(tetrahydro- | |
| | | 2H-pyran-4-yl)-1H-indol- | |
| | | 1-yl)-2- | |
| | | methylcyclopropyl)-1,2,4- | |
| | | oxadiazol-5(4H)-one | |
| 23 Isomer B (Daicel Chiralpak IG; CO₂:Et OH + 0.1%N H₃H₂O) | 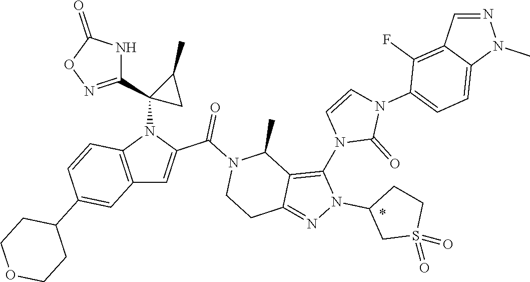 [SMILES: C[C@H]1c2c(nn(C3CCS(=O)(=O)C3)c2-n2ccn(-c3ccc4c(cnn4C)c3F)c2=O)CCN1C(=O)c1cc2cc(C3CCOCC3)ccc2n1[C@@]1(c2noc(=O)[nH]2)C[C@@H]1C] | 3-((1S,2S)-1-(2-((S)-2- ((S)-1,1- dioxidotetrahydrothiophen- 3-yl)-3-(3-(4-fluoro-1- methyl-1H-indazol-5-yl)- 2-oxo-2,3-dihydro-1H- imidazol-1-yl)-4-methyl- 4,5,6,7-tetrahydro-2H- pyrazolo[4,3-c]pyridine-5- carbonyl)-5-(tetrahydro- 2H-pyran-4-yl)-1H-indol- 1-yl)-2- methylcyclopropyl)-1,2,4- oxadiazol-5(4H)-one or 3- ((1S,2S)-1-(2-((S)-2-((R)- 1,1-dioxidotetrahydrothiophen- 3-yl)-3-(3-(4-fluoro-1- methyl-1H-indazol-5-yl)- | 851.2 |
| | | 2-oxo-2,3-dihydro-1H- | |
| | | imidazol-1-yl)-4-methyl- | |
| | | 4,5,6,7-tetrahydro-2H- | |
| | | pyrazolo[4,3-c]pyridine-5- | |
| | | carbonyl)-5-(tetrahydro- | |
| | | 2H-pyran-4-yl)-1H-indol- | |
| | | 1-yl)-2- | |
| | | methylcyclopropyl)-1,2,4- | |
| | | oxadiazol-5(4H)-one | |
| 24 Isomer A (Daicel Chiralcel OD-3; CO₂:Et OH + 0.05% DEA) | 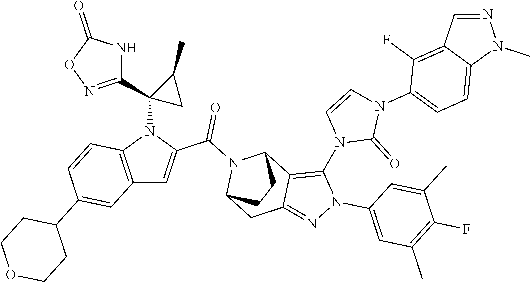 [SMILES: Cc1cc(-n2nc3c(c2-n2ccn(-c4ccc5c(cnn5C)c4F)c2=O)[C@@H]2CC[C@H](C3)N2C(=O)c2cc3cc(C4CCOCC4)ccc3n2[C@@]2(c3noc(=O)[nH]3)C[C@@H]2C)cc(C)c1F] | 3-((1S,2S)-1-(2-((4S,7R)- 3-(3-(4-fluoro-1-methyl- 1H-indazol-5-yl)-2-oxo- 2,3-dihydro-1H-imidazol- 1-yl)-2-(4-fluoro-3,5- dimethylphenyl)- 2,4,5,6,7,8-hexahydro-4,7- epiminocyclohepta[c] pyrazole-9-carbonyl)-5- (tetrahydro-2H-pyran-4- yl)-1H-indol-1-yl)-2- methylcyclopropyl)-1,2,4- oxadiazol-5(4H)-one OR 3-((1S,2S)-1-(2-((4R,7S)- 3-(3-(4-fluoro-1-methyl- 1H-indazol-5-yl)-2-oxo- 2,3-dihydro-1H-imidazol- | 867.3 |
| | | 1-yl)-2-(4-fluoro-3,5- | |
| | OR | dimethylphenyl)- | |
| | | 2,4,5,6,7,8-hexahydro-4,7- | |
| | 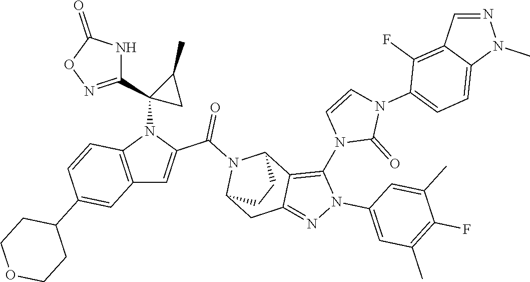 [SMILES: Cc1cc(-n2nc3c(c2-n2ccn(-c4ccc5c(cnn5C)c4F)c2=O)[C@H]2CC[C@@H](C3)N2C(=O)c2cc3cc(C4CCOCC4)ccc3n2[C@@]2(c3noc(=O)[nH]3)C[C@@H]2C)cc(C)c1F] | epiminocyclohepta[c] pyrazole-9-carbonyl)-5- (tetrahydro-2H-pyran-4- yl)-1H-indol-1-yl)-2- methylcyclopropyl)-1,2,4- oxadiazol-5(4H)-one | |
| 24 Isomer B (Chiralcel OD- 3; CO₂:Et OH + 0.05% DEA) | 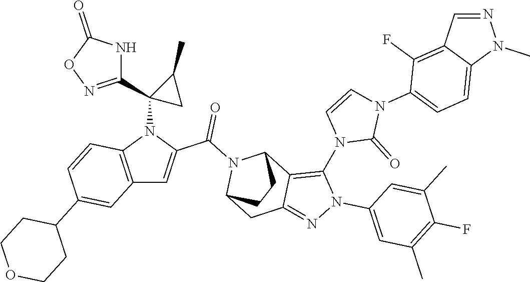 [SMILES: Cc1cc(-n2nc3c(c2-n2ccn(-c4ccc5c(cnn5C)c4F)c2=O)[C@@H]2CC[C@H](C3)N2C(=O)c2cc3cc(C4CCOCC4)ccc3n2[C@@]2(c3noc(=O)[nH]3)C[C@@H]2C)cc(C)c1F] | 3-((1S,2S)-1-(2-((4S,7R)- 3-(3-(4-fluoro-1-methyl- 1H-indazol-5-yl)-2-oxo- 2,3-dihydro-1H-imidazol- 1-yl)-2-(4-fluoro-3,5- dimethylphenyl)- 2,4,5,6,7,8-hexahydro-4,7- epiminocyclohepta[c]pyra zole-9-carbonyl)-5- (tetrahydro-2H-pyran-4- yl)-1H-indol-1-yl)-2- methylcyclopropyl)-1,2,4- oxadiazol-5(4H)-one OR 3-((1S,2S)-1-(2-((4R,7S)- 3-(3-(4-fluoro-1-methyl- 1H-indazol-5-yl)-2-oxo- 2,3-dihydro-1H-imidazol- | 867.3 |
| | | 1-yl)-2-(4-fluoro-3,5- | |
| | OR | dimethylphenyl)- | |
| | | 2,4,5,6,7,8-hexahydro-4,7- | |
| | 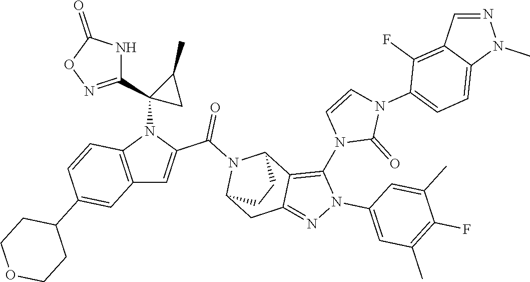 [SMILES: Cc1cc(-n2nc3c(c2-n2ccn(-c4ccc5c(cnn5C)c4F)c2=O)[C@H]2CC[C@@H](C3)N2C(=O)c2cc3cc(C4CCOCC4)ccc3n2[C@@]2(c3noc(=O)[nH]3)C[C@@H]2C)cc(C)c1F] | epiminocyclohepta[c] pyrazole-9-carbonyl)-5- (tetrahydro-2H-pyran-4- yl)-1H-indol-1-yl)-2- methylcyclopropyl)-1,2,4- oxadiazol-5(4H)-one | |
| 25 Isomer A (SiO₂ chrom.; Hexx: Et OAc) | 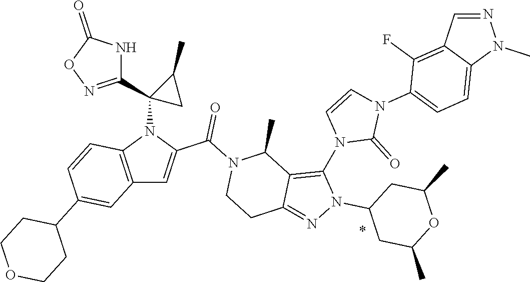 [SMILES: C[C@@H]1CC(n2nc3c(c2-n2ccn(-c4ccc5c(cnn5C)c4F)c2=O)[C@H](C)N(C(=O)c2cc4cc(C5CCOCC5)ccc4n2[C@@]2(c4noc(=O)[nH]4)C[C@@H]2C)CC3)C[C@H](C)O1] | 3-((1S,2S)-1-(2-((S)-2- ((2R,4r,6S)-2,6- dimethyltetrahydro-2H- pyran-4-yl)-3-(3-(4-fluoro- 1-methyl-1H-indazol-5- yl)-2-oxo-2,3-dihydro-1H- imidazol-1-yl)-4-methyl- 4,5,6,7-tetrahydro-2H- pyrazolo[4,3-c]pyridine-5- carbonyl)-5-(tetrahydro- 2H-pyran-4-yl)-1H-indol- 1-yl)-2- methylcyclopropyl)-1,2,4- oxadiazol-5(4H)-one or 3- ((1S,2S)-1-(2-((S)-2- ((2R,4s,6S)-2,6- dimethyltetrahydro-2H- | 845.5 |
| | | pyran-4-yl)-3-(3-(4-fluoro- | |
| | | 1-methyl-1H-indazol-5- | |
| | | yl)-2-oxo-2,3-dihydro-1H- | |
| | | imidazol-1-yl)-4-methyl- | |
| | | 4,5,6,7-tetrahydro-2H- | |
| | | pyrazolo[4,3-c]pyridine-5- | |
| | | carbonyl)-5-(tetrahydro- | |
| | | 2H-pyran-4-yl)-1H-indol- | |
| | | 1-yl)-2- | |
| | | methylcyclopropyl)-1,2,4- | |
| | | oxadiazol-5(4H)-one | |
| 25 Isomer B (SiO₂ chrom.; Hex:Et OAc) | 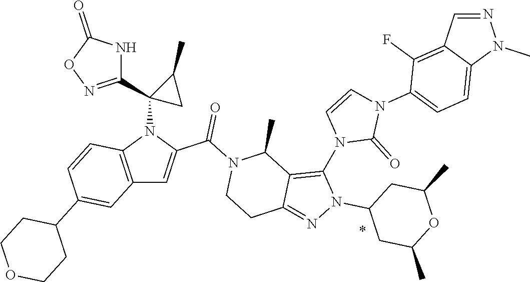 [SMILES: C[C@@H]1CC(n2nc3c(c2-n2ccn(-c4ccc5c(cnn5C)c4F)c2=O)[C@H](C)N(C(=O)c2cc4cc(C5CCOCC5)ccc4n2[C@@]2(c4noc(=O)[nH]4)C[C@@H]2C)CC3)C[C@H](C)O1] | 3-((1S,2S)-1-(2-((S)-2- ((2R,4r,6S)-2,6- dimethyltetrahydro-2H- pyran-4-yl)-3-(3-(4-fluoro- 1-methyl-1H-indazol-5- yl)-2-oxo-2,3-dihydro-1H- imidazol-1-yl)-4-methyl- 4,5,6,7-tetrahydro-2H- pyrazolo[4,3-c]pyridine-5- carbonyl)-5-(tetrahydro- 2H-pyran-4-yl)-1H-indol- 1-yl)-2- methylcyclopropyl)-1,2,4- oxadiazol-5(4H)-one or 3- ((1S,2S)-1-(2-((S)-2- ((2R,4s,6S)-2,6- dimethyltetrahydro-2H- | 845.5 |
| | | pyran-4-yl)-3-(3-(4-fluoro- | |
| | | 1-methyl-1H-indazol-5- | |
| | | yl)-2-oxo-2,3-dihydro-1H- | |
| | | imidazol-1-yl)-4-methyl- | |
| | | 4,5,6,7-tetrahydro-2H- | |
| | | pyrazolo[4,3-c]pyridine-5- | |
| | | carbonyl)-5-(tetrahydro- | |
| | | 2H-pyran-4-yl)-1H-indol- | |
| | | 1-yl)-2- | |
| | | methylcyclopropyl)-1,2,4- | |
| | | oxadiazol-5(4H)-one | |

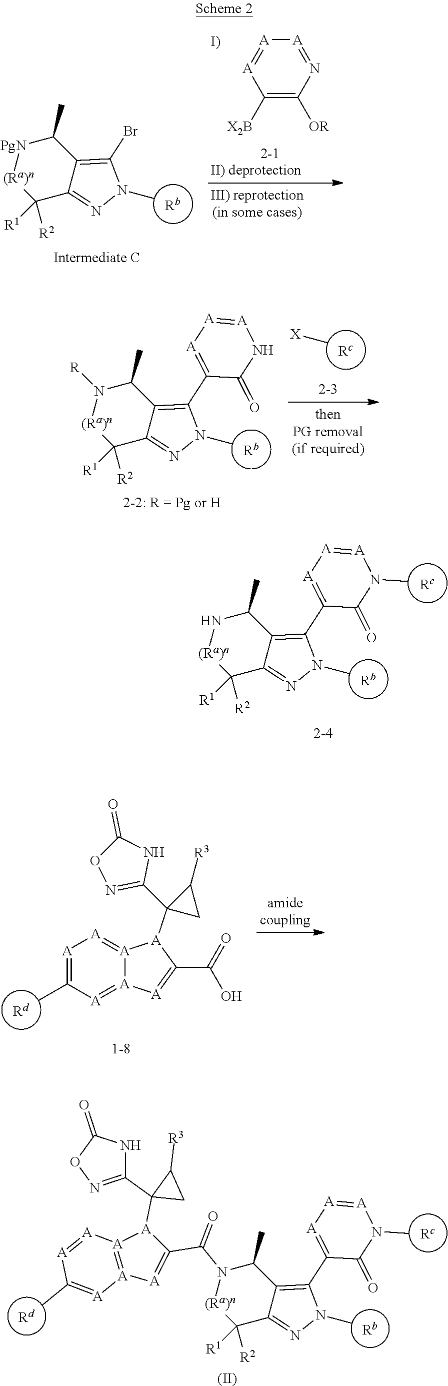

Compounds of formula (II) are prepared from intermediate C by coupling with heteroaryl boronic acid or other boronate partners (2-1) via transition metal catalysis (e.g., Pd-catalysis) and then removal of the protecting groups (that are present) via Lewis (e.g., MgI₂) or Bronsted (e.g., HCl) acids to provide intermediate and re-protection of the N atom (in some examples) to provide 2-2. Subsequently, 2-2 is coupled with halides, boronic acids, boronates, or sulfonates (2-3) via transition metal catalyzed cross-coupling (e.g., Pd—or Cu-catalysis) or alkylation (e.g., basic) conditions to furnish amine 2-4 after removal of the protecting group if required (e.g., acidic conditions). Then 2-4 is coupled with carboxylic acid 1-8 to provide compounds of formula (II).

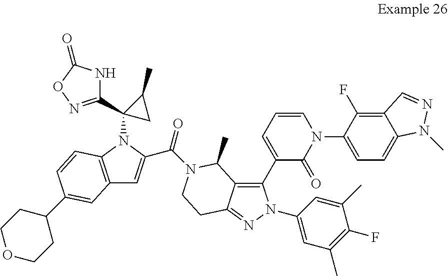 [SMILES: Cc1cc(-n2nc3c(c2-c2cccn(-c4ccc5c(cnn5C)c4F)c2=O)[C@H](C)N(C(=O)c2cc4cc(C5CCOCC5)ccc4n2[C@@]2(c4noc(=O)[nH]4)C[C@@H]2C)CC3)cc(C)c1F]

### 3-((1S,2S)-1-(2-((S)-3-(1-(4-fluoro-1-methyl-1H-indazol-5-yl)-2-oxo-1,2-dihydropyridin-3-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one (Scheme 2)

### Step 1: tert-butyl (S)-2-(4-fluoro-3,5-dimethylphenyl)-4-methyl-3-(2-oxo-1,2-dihydropyridin-3-yl)-2,4,6,7-tetrahydro-5H-pyrazolo[4,3-c]pyridine-5-carboxylate hydrochloride

To a solution of potassium phosphate tribasic (1.45 g, 6.84 mmol), (2-methoxypyridin-3-yl) boronic acid (0.349 g, 2.28 mmol) and tert-butyl (S)-3-bromo-2-(4-fluoro-3,5-dimethylphenyl)-4-methyl-2,4,6,7-tetrahydro-5H-pyrazolo[4,3-c]pyridine-5-carboxylate (1.00 g, 2.28 mmol) in t-AmOH (10.0 mL) and water (2.0 mL) was added dtbpf-Pd-G3 (0.199 g, 0.228 mmol) under an atmosphere of N₂. The reaction mixture was stirred at 60° C. for 16 h, then cooled to rt and extracted with EtOAc (3×15 mL). The combined organic layers were filtered and concentrated under reduced pressure. The crude residue was purified by silica gel chromatography (3:1 Pet.ether/EtOAc) to provide tert-butyl (S)-2-(4-fluoro-3,5-dimethylphenyl)-3-(2-methoxypyridin-3-yl)-4-methyl-2,4,6,7-tetrahydro-5H-pyrazolo[4,3-c]pyridine-5-carboxylate. MS (ESI) m/z: 467.8 [M+H+]. A solution of tert-butyl (S)-2-(4-fluoro-3,5-dimethylphenyl)-3-(2-methoxypyridin-3-yl)-4-methyl-2,4,6,7-tetrahydro-5H-pyrazolo[4,3-c]pyridine-5-carboxylate (1.6 g, 3.4 mmol) in 6M HCl (6.0 mL) and EtOAc (6.0 mL) was stirred at 40° C. for 16 h. The reaction mixture was concentrated under reduced pressure to provide (S)-3-(2-(4-fluoro-3,5-dimethylphenyl)-4-methyl-4,5,6,7-tetrahydro-2H-pyrazolo[4,3-c]pyridin-3-yl)pyridin-2(1H)-one hydrochloride. A solution of (S)-3-(2-(4-fluoro-3,5-dimethylphenyl)-4-methyl-4,5,6,7-tetrahydro-2H-pyrazolo[4,3-c]pyridin-3-yl)pyridin-2(1H)-one hydrochloride (1.2 g, 2.8 mmol), triethylamine (1.5 mL, 11 mmol) and di-tert-butyl dicarbonate (1.2 g, 5.6 mmol) in DCM (3.0 mL) was stirred at 25° C. for 16 h. The reaction mixture was concentrated under reduced pressure and purified by silica gel chromatography (33% to 100% EtOAc/Pet.ether) to provide the title compound. MS (ESI) m/z: 453.1 [M+H+]

### Step 2: tert-butyl (S)-3-(1-(4-fluoro-1-methyl-1H-indazol-5-yl)-2-oxo-1,2-dihydropyridin-3-yl)-2-(4-fluoro-3,5-dimethylphenyl)-4-methyl-2,4,6,7-tetrahydro-5H-pyrazolo[4,3-c]pyridine-5-carboxylate

A solution of tert-butyl (S)-2-(4-fluoro-3,5-dimethylphenyl)-4-methyl-3-(2-oxo-1,2-dihydropyridin-3-yl)-2,4,6,7-tetrahydro-5H-pyrazolo[4,3-c]pyridine-5-carboxylate (400 mg, 0.884 mmol), 4-fluoro-1-methyl-5-(4,4,5,5-tetramethyl-1,3,2-dioxaborolan-2-yl)-1H-indazole (488 mg, 1.77 mmol), pyridine (0.858 mL, 10.6 mmol) and copper(II) acetate (321 mg, 1.77 mmol) in DCM (2.0 mL) was stirred at 60° C. for 16 h under an atmosphere of O₂ (via balloon). Then, the reaction mixture was quenched with NH₄Cl (1 mL) and the mixture and stirred at rt for 10 min, filtered and concentrated under reduced pressure. The crude residue was purified by Prep-HPLC (55% to 75% MeCN/H₂O+0.1% TFA gradient) to provide the title compound. MS (ESI) m/z: 601.1 [M+H+]

### Step 3: (S)-1-(4-fluoro-1-methyl-1H-indazol-5-yl)-3-(2-(4-fluoro-3,5-dimethylphenyl)-4-methyl-4,5,6,7-tetrahydro-2H-pyrazolo[4,3-c]pyridin-3-yl)pyridin-2(1H)-one

A solution of tert-butyl (S)-3-(1-(4-fluoro-1-methyl-1H-indazol-5-yl)-2-oxo-1,2-dihydropyridin-3-yl)-2-(4-fluoro-3,5-dimethylphenyl)-4-methyl-2,4,6,7-tetrahydro-5H-pyrazolo[4,3-c]pyridine-5-carboxylate (25 mg, 0.042 mmol) in DCM (1.0 mL) and 2M HCl in dioxane (1.0 mL) was stirred at 25° C. for 2 h. Then, the reaction mixture was concentrated under reduced pressure to provide the title compound. MS (ESI) m/z: 501.1.

### Step 4: 3-((1S,2S)-1-(2-((S)-3-(1-(4-fluoro-1-methyl-1H-indazol-5-yl)-2-oxo-1,2-dihydropyridin-3-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one

To a solution of 1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carboxylic acid (18.38 mg, 0.048 mmol), (S)-1-(4-fluoro-1-methyl-1H-indazol-5-yl)-3-(2-(4-fluoro-3,5-dimethylphenyl)-4-methyl-4,5,6,7-tetrahydro-2H-pyrazolo[4,3-c]pyridin-3-yl)pyridin-2(1H)-one (20 mg, 0.040 mmol) and DIPEA (0.028 mL, 0.16 mmol) in DMF (1.0 mL) was added HATU (23 mg, 0.060 mmol) at 25° C. under an atmosphere of N₂. The reaction mixture was stirred at 25° C. for 16 h. Then, the reaction mixture was purified by reverse phase prep-HPLC (50% to 70% MeCN/H₂O+0.1% TFA gradient) to provide the title compound as the TFA salt. ¹H NMR (methanol-d4, 400 MHz) δ 8.20 (s, 0.5H), 7.72-7.78 (m, 0.5H), 7.21-7.72 (m, 7H), 7.10-7.15 (m, 1H), 7.0.1-7.06 (m, 1H), 6.80-6.87 (m, 1H), 6.51-6.66 (m, 0.5H,), 6.21-6.42 (m, 0.5H), 5.60-5.74 (m, 0.5H), 5.31-5.52 (m, 0.5H), 4.42-4.61 (m, 0.5H), 4.02-4.22 (m, 6H), 3.51-3.75 (m, 3H), 2.81-3.16 (m, 2H), 2.20-2.26 (m, 6H), 1.72-1.93 (m, 6H), 1.51-1.63 (m, 2H), 1.42-1.50 (m, 2H), 1.20-1.33 (m, 3H), 1.01-1.03 (m, 1H). MS (ESI) m/z: 866.2 [M+H+].

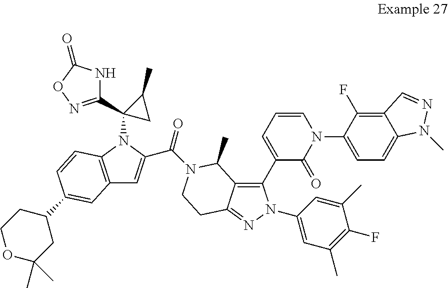

### 3-((1S,2S)-1-(5-((S)-2,2-dimethyltetrahydro-2H-pyran-4-yl)-2-((S)-3-(1-(4-fluoro-1-methyl-1H-indazol-5-yl)-2-oxo-1,2-dihydropyridin-3-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one

The steps were performed in a similar fashion as described for example 26 to provide the title compound as the TFA salt. ¹H NMR (methanol-d4, 400 MHz) δ 8.20-8.22 (m, 0.5H), 7.80-7.86 (m, 0.5H), 7.21-7.76 (m, 7H), 7.01-7.26 (m, 2H), 6.81-6.88 (m, 1H), 6.50-6.63 (m, 0.6H), 6.30-6.38 (m, 0.4H), 5.72-5.85 (m, 0.5H), 4.41-4.53 (m, 0.5H), 4.00-4.25 (m, 3H), 3.81-4.09 (m, 2H), 3.53-3.78 (m, 1H), 3.02-3.38 (m, 2H), 2.91-3.08 (m, 1H), 2.20-2.60 (m, 6H), 1.71-1.95 (m, 4H), 1.51-1.73 (m, 4H), 1.37-1.58 (m, 6H), 1.30-1.36 (m, 3H), 1.21-1.28 (m, 2H), 0.80-1.13 (m, 2H). MS (ESI) m/z: 894.3 [M+H]+.

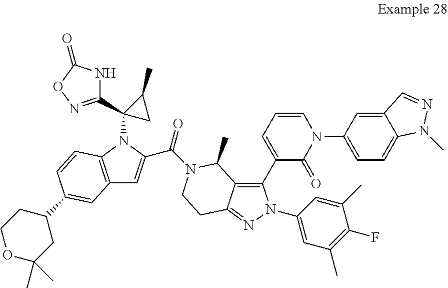

### 3-((1S,2S)-1-(5-((S)-2,2-dimethyltetrahydro-2H-pyran-4-yl)-2-((S)-2-(4-fluoro-3,5-dimethylphenyl)-4-methyl-3-(1-(1-methyl-1H-indazol-5-yl)-2-oxo-1,2-dihydropyridin-3-yl)-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one (scheme 2)

### Step 1: Same as Step 1 of Example 26

### Step 2: tert-butyl (S)-2-(4-fluoro-3,5-dimethylphenyl)-4-methyl-3-(1-(1-methyl-1H-indazol-5-yl)-2-oxo-1,2-dihydropyridin-3-yl)-2,4,6,7-tetrahydro-5H-pyrazolo[4,3-c]pyridine-5-carboxylate

To a solution of tert-butyl (S)-2-(4-fluoro-3,5-dimethylphenyl)-4-methyl-3-(2-oxo-1,2-dihydropyridin-3-yl)-2,4,6,7-tetrahydro-5H-pyrazolo[4,3-c]pyridine-5-carboxylate (500 mg, 0.552 mmol), 5-bromo-1-methyl-1H-indazole (233 mg, 1.11 mmol), potassium phosphate tribasic (352 mg, 1.66 mmol) and N1,N2-dimethylethane-1,2-diamine (97 mg, 1.11 mmol) in dioxane (5.0 mL) was added copper(I) iodide (105 mg, 0.552 mmol) at 25° C. under an atmosphere of nitrogen. The mixture was stirred at 110° C. for 16 h. The reaction mixture was cooled to rt, ammonium hydroxide (91 mL) was added. Then, the resulting mixture was stirred at rt for 10 min, filtered, and the filtrate was concentrated under reduced pressure. The crude residue was purified by prep-TLC (SiO₂, Pet. Ether/EtOAc=1:1) to provide the title compound. MS (ESI) m/z: 583.4 [M+H]+.

### Steps 3-4: 3-((1S,2S)-1-(5-((S)-2,2-dimethyltetrahydro-2H-pyran-4-yl)-2-((S)-2-(4-fluoro-3,5-dimethylphenyl)-4-methyl-3-(1-(1-methyl-1H-indazol-5-yl)-2-oxo-1,2-dihydropyridin-3-yl)-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one

The steps were performed in a similar fashion as described for example 26 to provide the title compound as the TFA salt. ¹H NMR (400 MHz, acetonitrile-d3) δ 11.62 (s, 0.7H),11.54 (s, 0.3H), 8.06 (s, 0.7H), 7.97 (s, 0.3H), 7.61-7.77 (m, 2H), 7.45-7.56 (m, 3H), 7.20-7.36 (m, 2H), 7.11 (d, J=6.4 Hz, 1.4H), 7.02 (d, J=6.4 Hz, 0.6H), 6.84 (s, 0.7H), 6.79 (s, 0.3H), 6.36 (t, J=6.8 Hz, 0.7H), 6.17 (t, J=6.8 Hz, 0.3H), 5.82 (m, 0.7H), 5.29 (m, 0.3H), 4.79 (dd, J=5.4, 13.2 Hz, 0.3H), 4.40 (dd, J=5.2, 13.2 Hz, 0.7H), 4.03-4.14 (m, 3H), 3.71-3.86 (m, 2H), 3.49-3.68 (m, 1H), 2.99-3.23 (m, 2H), 2.87-2.98 (m, 1H), 2.14-2.32 (m, 6H), 1.78-1.86 (m, 1H), 1.49-1.76 (m, 8H), 1.42 (d, J=6.4 Hz, 2H), 1.27-1.35 (m, 3H), 1.21 (s, 3H), 1.15 (d, J=6.0 Hz, 2H), 0.95 (d, J=6.0 Hz, 1H). MS (ESI) m/z: 876.7 [M+H]+.

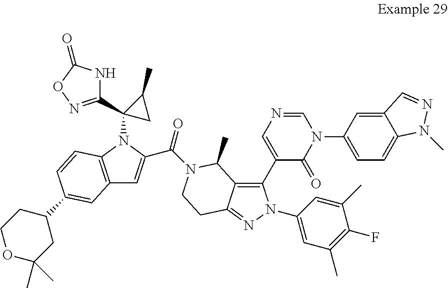

### 3-((1S,2S)-1-(5-((S)-2,2-dimethyltetrahydro-2H-pyran-4-yl)-2-((S)-2-(4-fluoro-3,5-dimethylphenyl)-4-methyl-3-(1-(1-methyl-1H-indazol-5-yl)-6-oxo-1,6-dihydropyrimidin-5-yl)-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one (scheme 2)

### Step 1: (S)-5-(2-(4-fluoro-3,5-dimethylphenyl)-4-methyl-4,5,6,7-tetrahydro-2H-pyrazolo[4,3-c]pyridin-3-yl)pyrimidin-4(3H)-one

To a mixture of potassium phosphate tribasic (145 mg, 0.684 mmol), dtbpf-Pd-G3 (19.3 mg 0.023 mmol), (4-methoxypyrimidin-5-yl) boronic acid (52.7 mg, 0.342 mmol) and tert-butyl (S)-3-bromo-2-(4-fluoro-3,5-dimethylphenyl)-4-methyl-2,4,6,7-tetrahydro-5H-pyrazolo[4,3-c]pyridine-5-carboxylate (100 mg, 0.228 mmol) in t-AmOH (1.9 mL) and water (0.30 mL) was sparged with argon for 5 min. The reaction mixture was then stirred under argon at 100° C. for 2.5 h. The reaction mixture was cooled down, treated with additional (4-methoxypyrimidin-5-yl)boronic acid (35.1 mg, 0.228 mmol), then stirred at 100° C. for 20 min. The reaction mixture was cooled down, treated with additional dtbpf-Pd-G3 and stirred at 100° C. for 1 h. The reaction mixture was cooled down, concentrated and purified via silica gel chromatography (0-20% MeOH/DCM) to provide tert-butyl (4S)-2-(4-fluoro-3,5-dimethylphenyl)-3-(4-methoxypyrimidin-5-yl)-4-methyl-2,4,6,7-tetrahydro-5H-pyrazolo[4,3-c]pyridine-5-carboxylate. MS (ESI) m/z: 468.4 [M+H]+.

A mixture of tert-butyl (4S)-2-(4-fluoro-3,5-dimethylphenyl)-3-(4-methoxypyrimidin-5-yl)-4-methyl-2,4,6,7-tetrahydro-5H-pyrazolo[4,3-c]pyridine-5-carboxylate (100 mg, 0.214 mmol) in acetic acid (0.71 mL) was treated with HBr (262 mg, 0.18 mL, 1.07 mmol, 33% wt in acetic acid) and stirred at 60° C. for 2 h. The reaction mixture was then diluted with methanol, concentrated under reduced pressure, treated with DCM, sonicated, and concentrated under reduced pressure again to afford the title compound as the HBr salt, which was used further without purification. MS (ESI) m/z: 354.4 [M+H]+.

### Step 2: (S)-5-(2-(4-fluoro-3,5-dimethylphenyl)-4-methyl-4,5,6,7-tetrahydro-2H-pyrazolo[4,3-c]pyridin-3-yl)-3-(1-methyl-1H-indazol-5-yl)pyrimidin-4(3H)-one

A mixture of (S)-5-(2-(4-fluoro-3,5-dimethylphenyl)-4-methyl-4,5,6,7-tetrahydro-2H-pyrazolo[4,3-c]pyridin-3-yl)-3-(1-methyl-1H-indazol-5-yl)pyrimidin-4(3H)-one (HBr salt, 73 mg, 0.17 mmol), (1-methyl-1H-indazol-5-yl)boronic acid (44 mg, 0.25 mmol) and copper(II) acetate (61 mg, 0.34 mmol) in pyridine (0.84 mL) was stirred under air at rt for 4 days. The reaction mixture was then treated with additional (1-methyl-1H-indazol-5-yl)boronic acid and stirred under air at 70° C. for 1 h. The reaction mixture was then concentrated and purified via C18 reverse phase column chromatography (10-60% MeCN/water+0.1% TFA modifier) to afford the title compound as the TFA salt. MS (ESI) m/z: 484.4 [M+H]+.

### Step 3: 3-((1S,2S)-1-(5-((S)-2,2-dimethyltetrahydro-2H-pyran-4-yl)-2-((S)-2-(4-fluoro-3,5-dimethylphenyl)-4-methyl-3-(1-(1-methyl-1H-indazol-5-yl)-6-oxo-1,6-dihydropyrimidin-5-yl)-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one

A mixture of (S)-5-(2-(4-fluoro-3,5-dimethylphenyl)-4-methyl-4,5,6,7-tetrahydro-2H-pyrazolo[4,3-c]pyridin-3-yl)-3-(1-methyl-1H-indazol-5-yl)pyrimidin-4(3H)-one (TFA salt, 41 mg, 0.069 mmol), HATU (39 mg, 0.10 mmol) and 5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carboxylic acid (42 mg, 0.10 mmol) in DMF (0.46 mL) was treated with DIPEA (44 mg, 60 μL, 0.34 mmol) and stirred at room temperature for 2 h. The reaction mixture was then directly purified via C18 reverse phase column chromatography (10-100% MeCN/water+0.1% formic acid modifier) to provide the title compound as a solid. ¹H NMR (500 MHz, CD₃CN) δ 8.40-8.20 (m, 1H), 8.17-8.01 (m, 1H), 7.98-7.68 (m, 2H), 7.61-7.36 (m, 3H), 7.35-7.08 (m, 3H), 7.08-6.88 (m, 1H), 6.90-6.77 (m, 1H), 5.92-5.30 (m, 1H), 4.86-4.34 (m, 1H), 4.16-4.04 (m, 3H), 3.85-3.73 (m, 2H), 3.70-3.51 (m, 1H), 3.25-3.02 (m, 2H), 3.02-2.90 (m, 1H), 2.33-2.25 (m, 6H), 1.85-1.56 (m, 7H), 1.52-1.38 (m, 3H), 1.35-1.29 (m, 3H), 1.22 (s, 3H), 1.20-0.97 (m, 3H). MS (ESI) m/z: 877.6 [M+H]+.

The following examples in table 2 were prepared according to scheme 2 using the procedures outlined in the synthesis of examples 26, 27, 28, and 29 recited above.

TABLE 2

| Ex. | Structure |
|---|---|
| 30 | 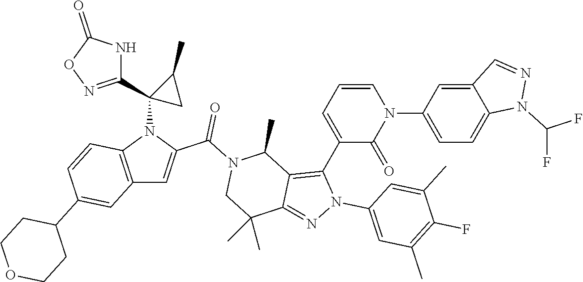 [SMILES: Cc1cc(-n2nc3c(c2-c2cccn(-c4ccc5c(cnn5C(F)F)c4)c2=O)[C@H](C)N(C(=O)c2cc4cc(C5CCOCC5)ccc4n2[C@@]2(c4noc(=O)[nH]4)C[C@@H]2C)CC3(C)C)cc(C)c1F] |
| 31 | 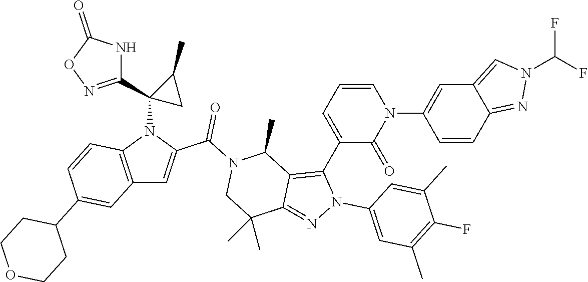 [SMILES: Cc1cc(-n2nc3c(c2-c2cccn(-c4ccc5nn(C(F)F)cc5c4)c2=O)[C@H](C)N(C(=O)c2cc4cc(C5CCOCC5)ccc4n2[C@@]2(c4noc(=O)[nH]4)C[C@@H]2C)CC3(C)C)cc(C)c1F] |
| 32 | 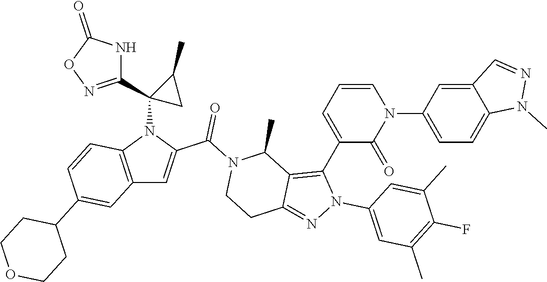 [SMILES: Cc1cc(-n2nc3c(c2-c2cccn(-c4ccc5c(cnn5C)c4)c2=O)[C@H](C)N(C(=O)c2cc4cc(C5CCOCC5)ccc4n2[C@@]2(c4noc(=O)[nH]4)C[C@@H]2C)CC3)cc(C)c1F] |
| 33 | 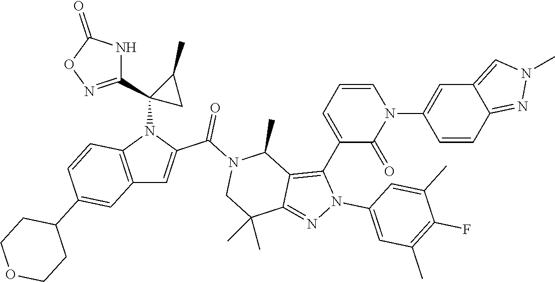 [SMILES: Cc1cc(-n2nc3c(c2-c2cccn(-c4ccc5nn(C)cc5c4)c2=O)[C@H](C)N(C(=O)c2cc4cc(C5CCOCC5)ccc4n2[C@@]2(c4noc(=O)[nH]4)C[C@@H]2C)CC3(C)C)cc(C)c1F] |
| 34 | 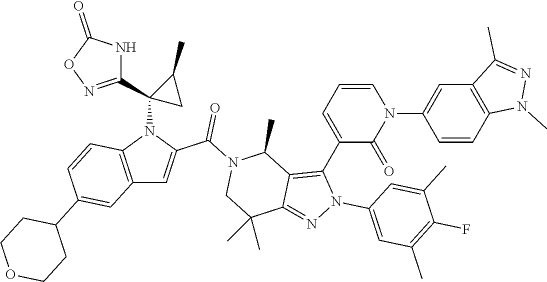 [SMILES: Cc1cc(-n2nc3c(c2-c2cccn(-c4ccc5c(c4)c(C)nn5C)c2=O)[C@H](C)N(C(=O)c2cc4cc(C5CCOCC5)ccc4n2[C@@]2(c4noc(=O)[nH]4)C[C@@H]2C)CC3(C)C)cc(C)c1F] |
| 35 | 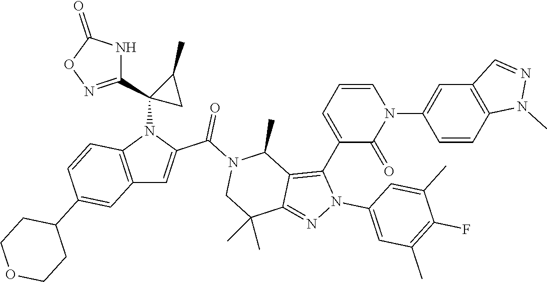 [SMILES: Cc1cc(-n2nc3c(c2-c2cccn(-c4ccc5c(cnn5C)c4)c2=O)[C@H](C)N(C(=O)c2cc4cc(C5CCOCC5)ccc4n2[C@@]2(c4noc(=O)[nH]4)C[C@@H]2C)CC3(C)C)cc(C)c1F] |
| 36 | 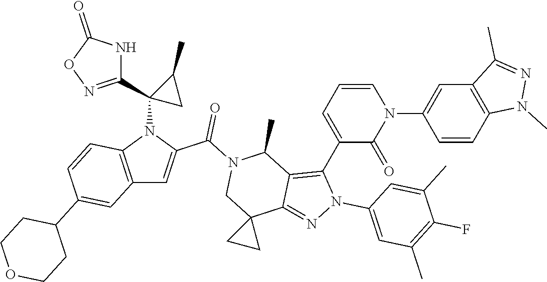 [SMILES: Cc1cc(-n2nc3c(c2-c2cccn(-c4ccc5c(c4)c(C)nn5C)c2=O)[C@H](C)N(C(=O)c2cc4cc(C5CCOCC5)ccc4n2[C@@]2(c4noc(=O)[nH]4)C[C@@H]2C)CC32CC2)cc(C)c1F] |
| 37 | 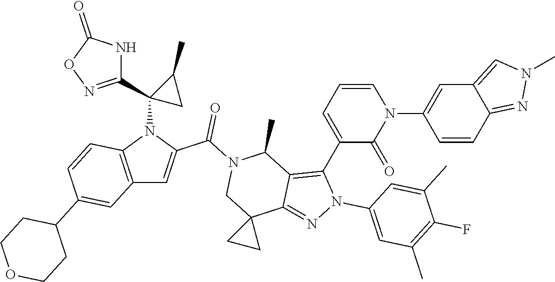 [SMILES: Cc1cc(-n2nc3c(c2-c2cccn(-c4ccc5nn(C)cc5c4)c2=O)[C@H](C)N(C(=O)c2cc4cc(C5CCOCC5)ccc4n2[C@@]2(c4noc(=O)[nH]4)C[C@@H]2C)CC32CC2)cc(C)c1F] |
| 38 | 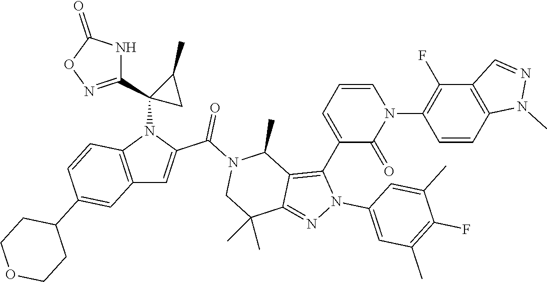 [SMILES: Cc1cc(-n2nc3c(c2-c2cccn(-c4ccc5c(cnn5C)c4F)c2=O)[C@H](C)N(C(=O)c2cc4cc(C5CCOCC5)ccc4n2[C@@]2(c4noc(=O)[nH]4)C[C@@H]2C)CC3(C)C)cc(C)c1F] |
| 39 | 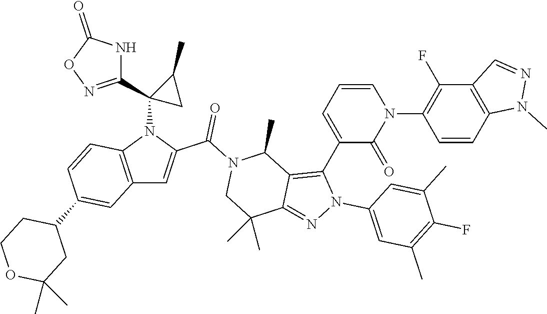 |
| 40 | 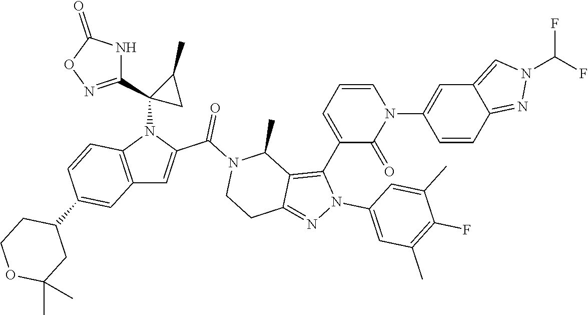 |
| 41 | 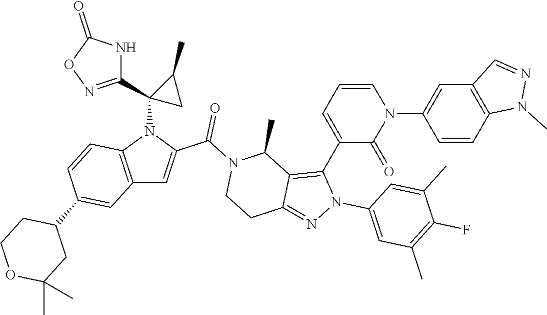 |
| 42 | 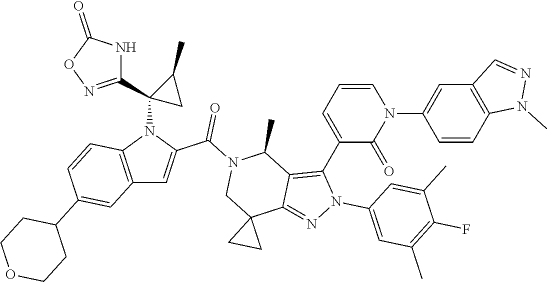 [SMILES: Cc1cc(-n2nc3c(c2-c2cccn(-c4ccc5c(cnn5C)c4)c2=O)[C@H](C)N(C(=O)c2cc4cc(C5CCOCC5)ccc4n2[C@@]2(c4noc(=O)[nH]4)C[C@@H]2C)CC32CC2)cc(C)c1F] |
| 43 | 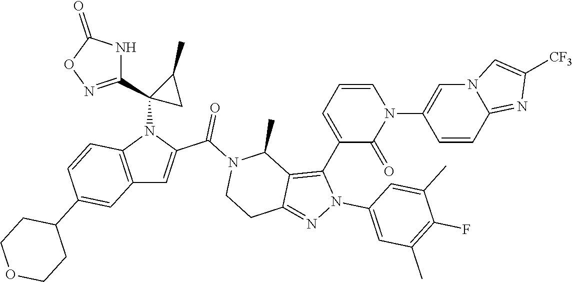 [SMILES: Cc1cc(-n2nc3c(c2-c2cccn(-c4ccc5nc(C(F)(F)F)cn5c4)c2=O)[C@H](C)N(C(=O)c2cc4cc(C5CCOCC5)ccc4n2[C@@]2(c4noc(=O)[nH]4)C[C@@H]2C)CC3)cc(C)c1F] |
| 44 | 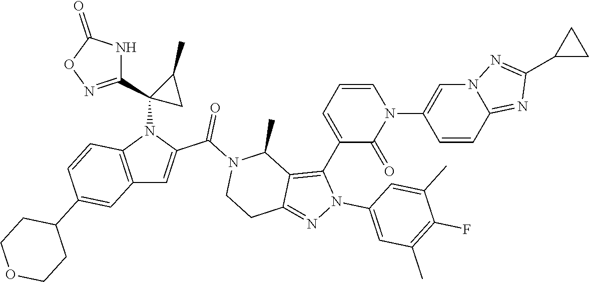 [SMILES: Cc1cc(-n2nc3c(c2-c2cccn(-c4ccc5nc(C6CC6)nn5c4)c2=O)[C@H](C)N(C(=O)c2cc4cc(C5CCOCC5)ccc4n2[C@@]2(c4noc(=O)[nH]4)C[C@@H]2C)CC3)cc(C)c1F] |
| 45 | 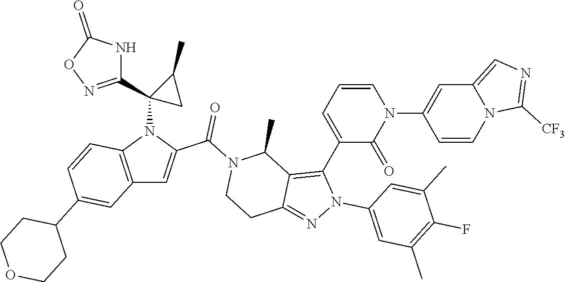 [SMILES: Cc1cc(-n2nc3c(c2-c2cccn(-c4ccn5c(C(F)(F)F)ncc5c4)c2=O)[C@H](C)N(C(=O)c2cc4cc(C5CCOCC5)ccc4n2[C@@]2(c4noc(=O)[nH]4)C[C@@H]2C)CC3)cc(C)c1F] |
| 46 | 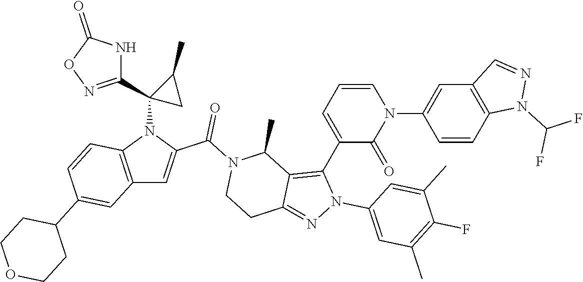 [SMILES: Cc1cc(-n2nc3c(c2-c2cccn(-c4ccc5c(cnn5C(F)F)c4)c2=O)[C@H](C)N(C(=O)c2cc4cc(C5CCOCC5)ccc4n2[C@@]2(c4noc(=O)[nH]4)C[C@@H]2C)CC3)cc(C)c1F] |
| 47 | 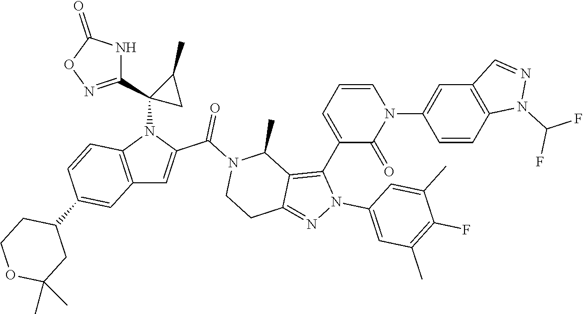 |
| 48 | 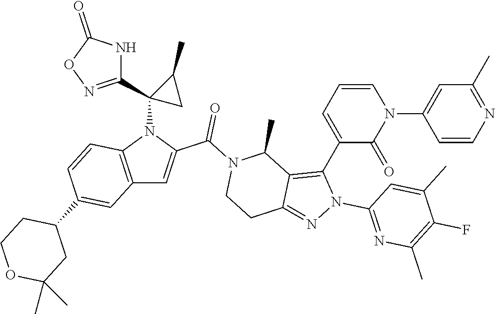 [SMILES: Cc1cc(-n2cccc(-c3c4c(nn3-c3cc(C)c(F)c(C)n3)CCN(C(=O)c3cc5cc([C@H]6CCOC(C)(C)C6)ccc5n3[C@@]3(c5noc(=O)[nH]5)C[C@@H]3C)[C@H]4C)c2=O)ccn1] |
| 49 | 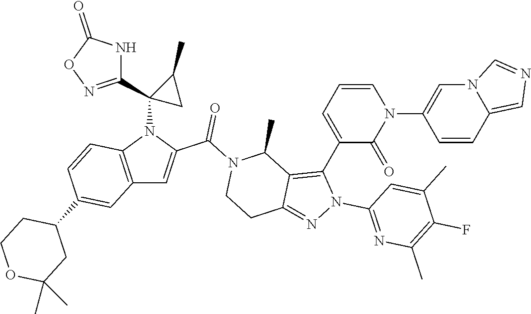 [SMILES: Cc1cc(-n2nc3c(c2-c2cccn(-c4ccc5cncn5c4)c2=O)[C@H](C)N(C(=O)c2cc4cc([C@H]5CCOC(C)(C)C5)ccc4n2[C@@]2(c4noc(=O)[nH]4)C[C@@H]2C)CC3)nc(C)c1F] |
| 50 | 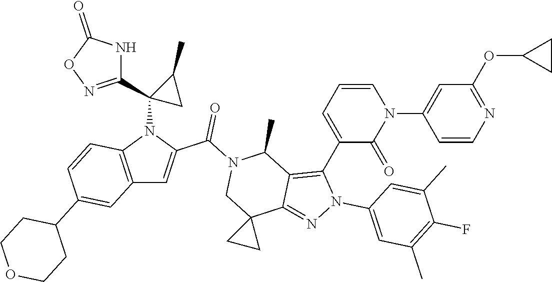 [SMILES: Cc1cc(-n2nc3c(c2-c2cccn(-c4ccnc(OC5CC5)c4)c2=O)[C@H](C)N(C(=O)c2cc4cc(C5CCOCC5)ccc4n2[C@@]2(c4noc(=O)[nH]4)C[C@@H]2C)CC32CC2)cc(C)c1F] |
| 51 | 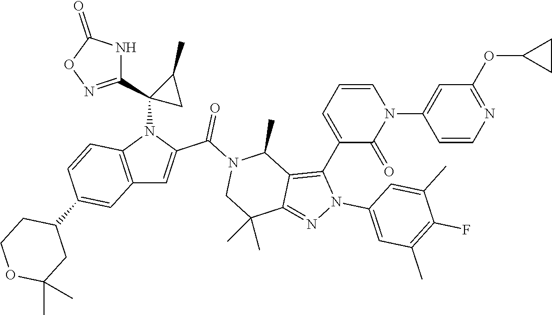 |
| 52 | 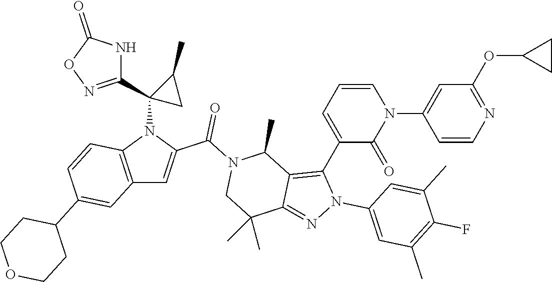 [SMILES: Cc1cc(-n2nc3c(c2-c2cccn(-c4ccnc(OC5CC5)c4)c2=O)[C@H](C)N(C(=O)c2cc4cc(C5CCOCC5)ccc4n2[C@@]2(c4noc(=O)[nH]4)C[C@@H]2C)CC3(C)C)cc(C)c1F] |
| 53 | 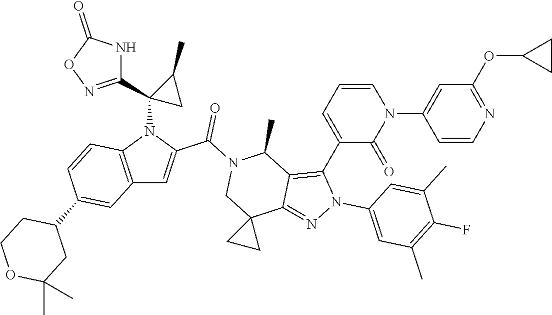 |
| 54 | 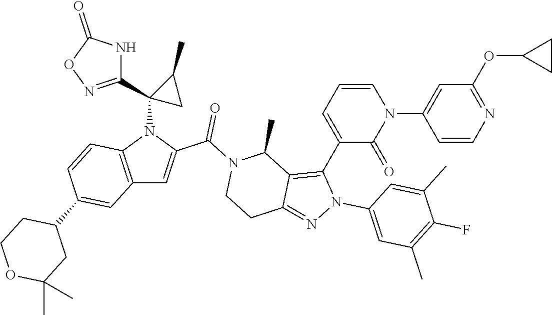 |
| 55 | 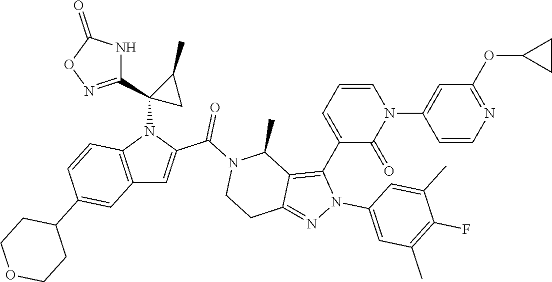 [SMILES: Cc1cc(-n2nc3c(c2-c2cccn(-c4ccnc(OC5CC5)c4)c2=O)[C@H](C)N(C(=O)c2cc4cc(C5CCOCC5)ccc4n2[C@@]2(c4noc(=O)[nH]4)C[C@@H]2C)CC3)cc(C)c1F] |
| 56 | 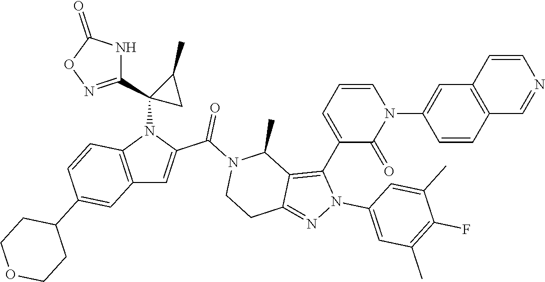 [SMILES: Cc1cc(-n2nc3c(c2-c2cccn(-c4ccc5cnccc5c4)c2=O)[C@H](C)N(C(=O)c2cc4cc(C5CCOCC5)ccc4n2[C@@]2(c4noc(=O)[nH]4)C[C@@H]2C)CC3)cc(C)c1F] |
| 57 | 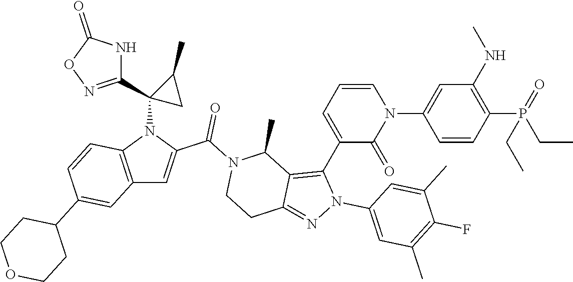 [SMILES: CCP(=O)(CC)c1ccc(-n2cccc(-c3c4c(nn3-c3cc(C)c(F)c(C)c3)CCN(C(=O)c3cc5cc(C6CCOCC6)ccc5n3[C@@]3(c5noc(=O)[nH]5)C[C@@H]3C)[C@H]4C)c2=O)cc1NC] |
| 58 | 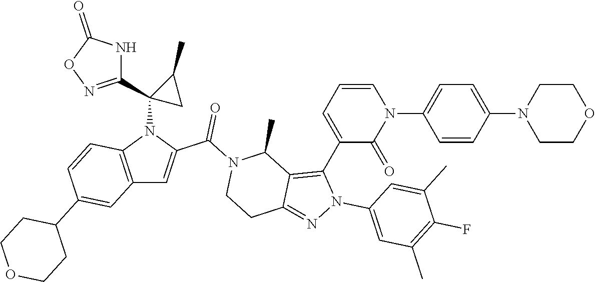 [SMILES: Cc1cc(-n2nc3c(c2-c2cccn(-c4ccc(N5CCOCC5)cc4)c2=O)[C@H](C)N(C(=O)c2cc4cc(C5CCOCC5)ccc4n2[C@@]2(c4noc(=O)[nH]4)C[C@@H]2C)CC3)cc(C)c1F] |
| 59 | 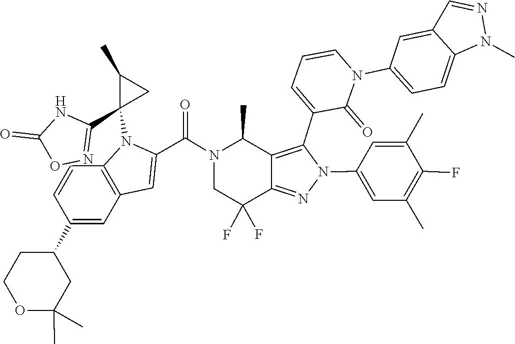 [SMILES: Cc1cc(-n2nc3c(c2-c2cccn(-c4ccc5c(cnn5C)c4)c2=O)[C@H](C)N(C(=O)c2cc4cc([C@H]5CCOC(C)(C)C5)ccc4n2[C@@]2(c4noc(=O)[nH]4)C[C@@H]2C)CC3(F)F)cc(C)c1F] |
| 60 | 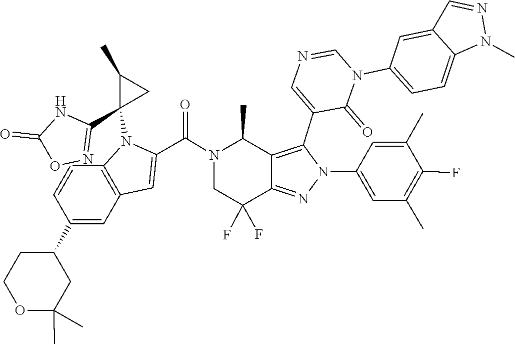 [SMILES: Cc1cc(-n2nc3c(c2-c2cncn(-c4ccc5c(cnn5C)c4)c2=O)[C@H](C)N(C(=O)c2cc4cc([C@H]5CCOC(C)(C)C5)ccc4n2[C@@]2(c4noc(=O)[nH]4)C[C@@H]2C)CC3(F)F)cc(C)c1F] |
| 61 | 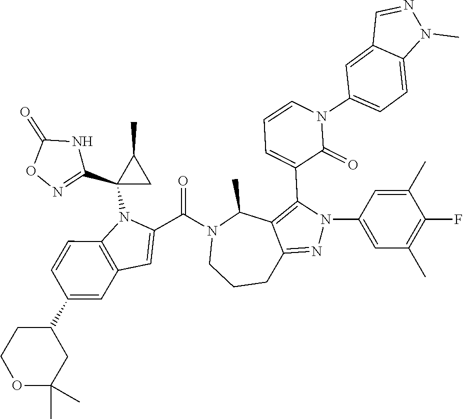 |
| 187 | 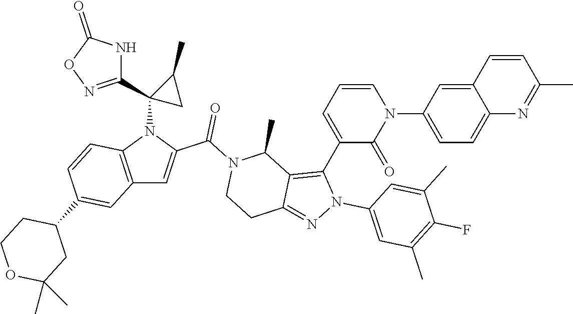 [SMILES: Cc1ccc2cc(-n3cccc(-c4c5c(nn4-c4cc(C)c(F)c(C)c4)CCN(C(=O)c4cc6cc([C@H]7CCOC(C)(C)C7)ccc6n4[C@@]4(c6noc(=O)[nH]6)C[C@@H]4C)[C@H]5C)c3=O)ccc2n1] |
| 188 | 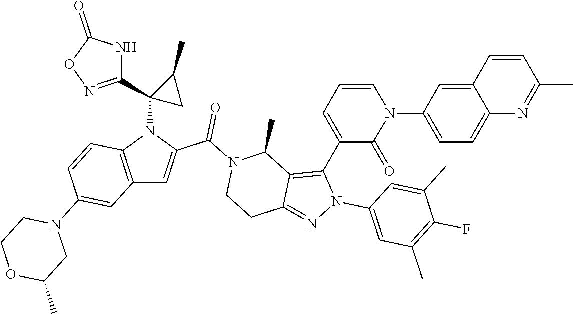 [SMILES: Cc1ccc2cc(-n3cccc(-c4c5c(nn4-c4cc(C)c(F)c(C)c4)CCN(C(=O)c4cc6cc(N7CCO[C@@H](C)C7)ccc6n4[C@@]4(c6noc(=O)[nH]6)C[C@@H]4C)[C@H]5C)c3=O)ccc2n1] |
| 189 | 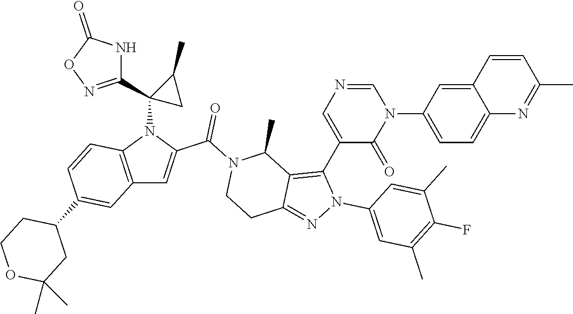 [SMILES: Cc1ccc2cc(-n3cncc(-c4c5c(nn4-c4cc(C)c(F)c(C)c4)CCN(C(=O)c4cc6cc([C@H]7CCOC(C)(C)C7)ccc6n4[C@@]4(c6noc(=O)[nH]6)C[C@@H]4C)[C@H]5C)c3=O)ccc2n1] |
| 190 | 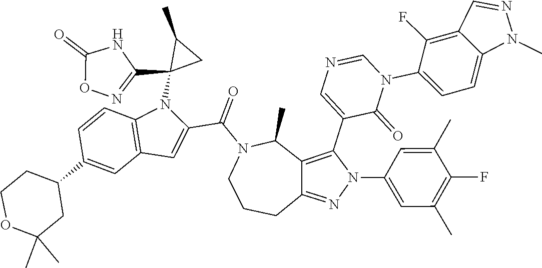 [SMILES: Cc1cc(-n2nc3c(c2-c2cncn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)C[C@@H]2C)CCC3)cc(C)c1F] |
| 191 | 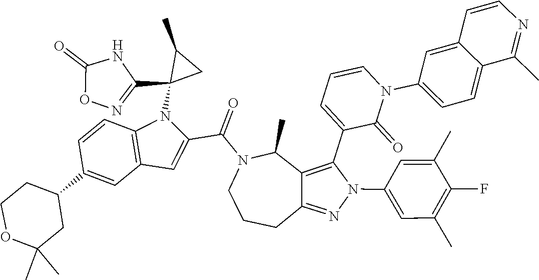 [SMILES: Cc1cc(-n2nc3c(c2-c2cccn(-c4ccc5c(C)nccc5c4)c2=O)[C@H](C)N(C(=O)c2cc4cc([C@H]5CCOC(C)(C)C5)ccc4n2[C@@]2(c4noc(=O)[nH]4)C[C@@H]2C)CCC3)cc(C)c1F] |
| 192 | 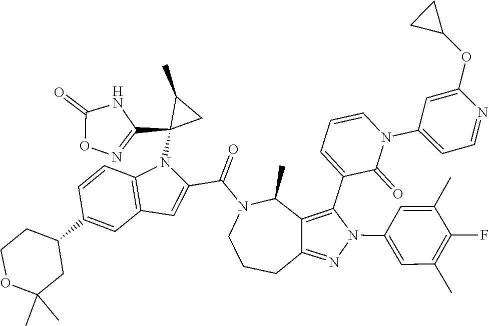 [SMILES: Cc1cc(-n2nc3c(c2-c2cccn(-c4ccnc(OC5CC5)c4)c2=O)[C@H](C)N(C(=O)c2cc4cc([C@H]5CCOC(C)(C)C5)ccc4n2[C@@]2(c4noc(=O)[nH]4)C[C@@H]2C)CCC3)cc(C)c1F] |
| 193 | 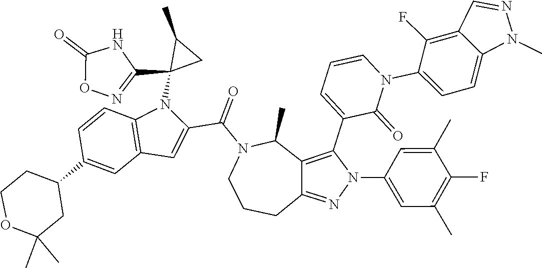 [SMILES: Cc1cc(-n2nc3c(c2-c2cccn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)C[C@@H]2C)CCC3)cc(C)c1F] |
| 194 | 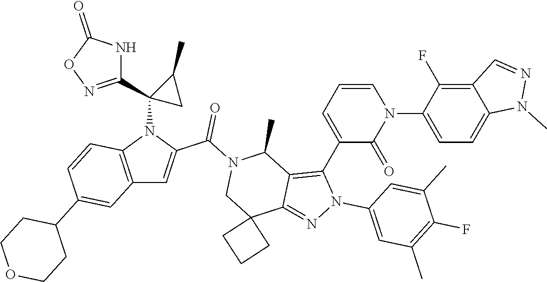 [SMILES: Cc1cc(-n2nc3c(c2-c2cccn(-c4ccc5c(cnn5C)c4F)c2=O)[C@H](C)N(C(=O)c2cc4cc(C5CCOCC5)ccc4n2[C@@]2(c4noc(=O)[nH]4)C[C@@H]2C)CC32CCC2)cc(C)c1F] |
| 195 | 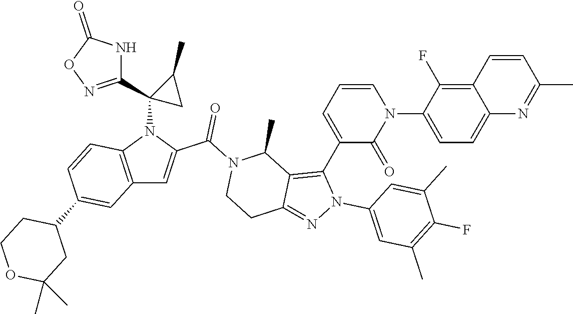 |
| 196 | 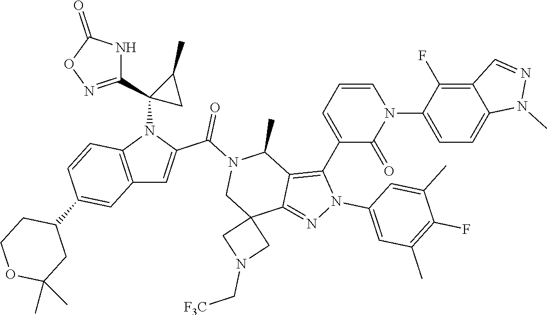 |
| 197 | 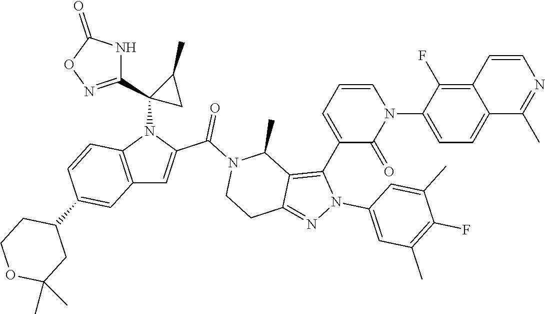 |
| 198 | 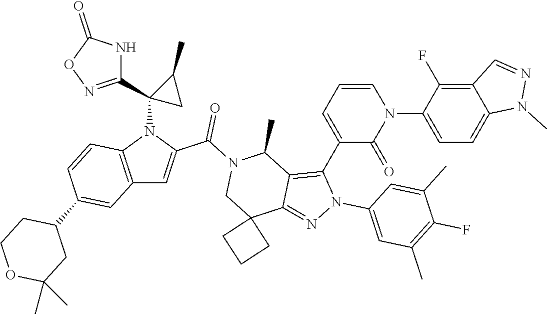 |

| | | MS |
|---|---|---|
| Ex. | Name | (M + 1) |
| 30 | 3-((1S,2S)-1-(2-((S)-3-(1-(1- | 912.3 |
| | (difluoromethyl)-1H-indazol-5-yl)- | |
| | 2-oxo-1,2-dihydropyridin-3-yl)-2- | |
| | (4-fluoro-3,5-dimethylphenyl)- | |
| | 4,7,7-trimethyl-4,5,6,7-tetrahydro- | |
| | 2H-pyrazolo[4,3-c]pyridine-5- | |
| | carbonyl)-5-(tetrahydro-2H-pyran- | |
| | 4-yl)-1H-indol-1-yl)-2- | |
| | methylcyclopropyl)-1,2,4- | |
| | oxadiazol-5(4H)-one | |
| 31 | 3-((1S,2S)-1-(2-((S)-3-(1-(2- | 912.3 |
| | (difluoromethyl)-2H-indazol-5-yl)- | |
| | 2-oxo-1,2-dihydropyridin-3-yl)-2- | |
| | (4-fluoro-3,5-dimethylphenyl)- | |
| | 4,7,7-trimethyl-4,5,6,7-tetrahydro- | |
| | 2H-pyrazolo[4,3-c]pyridine-5- | |
| | carbonyl)-5-(tetrahydro-2H-pyran- | |
| | 4-yl)-1H-indol-1-yl)-2- | |
| | methylcyclopropyl)-1,2,4- | |
| | oxadiazol-5(4H)-one | |
| 32 | 3-((1S,2S)-1-(2-((S)-2-(4-fluoro- | 848.6 |
| | 3,5-dimethylphenyl)-4-methyl-3- | |
| | (1-(1-methyl-1H-indazol-5-yl)-2- | |
| | oxo-1,2-dihydropyridin-3-yl)- | |
| | 4,5,6,7-tetrahydro-2H- | |
| | pyrazolo[4,3-c]pyridine-5- | |
| | carbonyl)-5-(tetrahydro-2H-pyran- | |
| | 4-yl)-1H-indol-1-yl)-2- | |
| | methylcyclopropyl)-1,2,4- | |
| | oxadiazol-5(4H)-one | |
| 33 | 3-((1S,2S)-1-(2-((S)-2-(4-fluoro- | 876.2 |
| | 3,5-dimethylphenyl)-4,7,7- | |
| | trimethyl-3-(1-(2-methyl-2H- | |
| | indazol-5-yl)-2-oxo-1,2- | |
| | dihydropyridin-3-yl)-4,5,6,7- | |
| | tetrahydro-2H-pyrazolo[4,3- | |
| | c]pyridine-5-carbonyl)-5- | |
| | (tetrahydro-2H-pyran-4-yl)-1H- | |
| | indol-1-yl)-2-methylcyclopropyl)- | |
| | 1,2,4-oxadiazol-5(4H)-one | |
| 34 | 3-((1S,2S)-1-(2-((S)-3-(1-(1,3- | 890.3 |
| | dimethyl-1H-indazol-5-yl)-2-oxo- | |
| | 1,2-dihydropyridin-3-yl)-2-(4- | |
| | fluoro-3,5-dimethylphenyl)-4,7,7- | |
| | trimethyl-4,5,6,7-tetrahydro-2H- | |
| | pyrazolo[4,3-c]pyridine-5- | |
| | carbonyl)-5-(tetrahydro-2H-pyran- | |
| | 4-yl)-1H-indol-1-yl)-2- | |
| | methylcyclopropyl)-1,2,4- | |
| | oxadiazol-5(4H)-one | |
| 35 | 3-((1S,2S)-1-(2-((S)-2-(4-fluoro- | 876.6 |
| | 3,5-dimethylphenyl)-4,7,7- | |
| | trimethyl-3-(1-(1-methyl-1H- | |
| | indazol-5-yl)-2-oxo-1,2- | |
| | dihydropyridin-3-yl)-4,5,6,7- | |
| | tetrahydro-2H-pyrazolo[4,3- | |
| | c]pyridine-5-carbonyl)-5- | |
| | (tetrahydro-2H-pyran-4-yl)-1H- | |
| | indol-1-yl)-2-methylcyclopropyl)- | |
| | 1,2,4-oxadiazol-5(4H)-one | |
| 36 | 3-((1S,2S)-1-(2-((S)-3′-(1-(1,3- | 888.2 |
| | dimethyl-1H-indazol-5-yl)-2-oxo- | |
| | 1,2-dihydropyridin-3-yl)-2′-(4- | |
| | fluoro-3,5-dimethylphenyl)-4′- | |
| | methyl-2′,4′,5′,6′- | |
| | tetrahydrospiro[cyclopropane-1,7′- | |
| | pyrazolo[4,3-c]pyridine]-5′- | |
| | carbonyl)-5-(tetrahydro-2H-pyran- | |
| | 4-yl)-1H-indol-1-yl)-2- | |
| | methylcyclopropyl)-1,2,4- | |
| | oxadiazol-5(4H)-one | |
| 37 | 3-((1S,2S)-1-(2-((S)-2′-(4-fluoro- | 874.3 |
| | 3,5-dimethylphenyl)-4′-methyl-3′- | |
| | (1-(2-methyl-2H-indazol-5-yl)-2- | |
| | oxo-1,2-dihydropyridin-3-yl)- | |
| | 2′,4′,5′,6′- | |
| | tetrahydrospiro[cyclopropane-1,7′- | |
| | pyrazolo[4,3-c]pyridine]-5′- | |
| | carbonyl)-5-(tetrahydro-2H-pyran- | |
| | 4-yl)-1H-indol-1-yl)-2- | |
| | methylcyclopropyl)-1,2,4- | |
| | oxadiazol-5(4H)-one | |
| 38 | 3-((1S,2S)-1-(2-((S)-3-(1-(4- | 894.1 |
| | fluoro-1-methyl-1H-indazol-5-yl)- | |
| | 2-oxo-1,2-dihydropyridin-3-yl)-2- | |
| | (4-fluoro-3,5-dimethylphenyl)- | |
| | 4,7,7-trimethyl-4,5,6,7-tetrahydro- | |
| | 2H-pyrazolo[4,3-c]pyridine-5- | |
| | carbonyl)-5-(tetrahydro-2H-pyran- | |
| | 4-yl)-1H-indol-1-yl)-2- | |
| | methylcyclopropyl)-1,2,4- | |
| | oxadiazol-5(4H)-one | |
| 39 | 3-((1S,2S)-1-(5-((S)-2,2- | 922.4 |
| | dimethyltetrahydro-2H-pyran-4- | |
| | yl)-2-((S)-3-(1-(4-fluoro-1-methyl- | |
| | 1H-indazol-5-yl)-2-oxo-1,2- | |
| | dihydropyridin-3-yl)-2-(4-fluoro- | |
| | 3,5-dimethylphenyl)-4,7,7- | |
| | trimethyl-4,5,6,7-tetrahydro-2H- | |
| | pyrazolo[4,3-c]pyridine-5- | |
| | carbonyl)-1H-indol-1-yl)-2- | |
| | methylcyclopropyl)-1,2,4- | |
| | oxadiazol-5(4H)-one | |
| 40 | 3-((1S,2S)-1-(2-((S)-3-(1-(2- | 941.1 |
| | (difluoromethyl)-2H-indazol-5-yl)- | |
| | 2-oxo-1,2-dihydropyridin-3-yl)-2- | |
| | (4-fluoro-3,5-dimethylphenyl)- | |
| | 4,7,7-trimethyl-4,5,6,7-tetrahydro- | |
| | 2H-pyrazolo[4,3-c]pyridine-5- | |
| | carbonyl)-5-((S)-2,2- | |
| | dimethyltetrahydro-2H-pyran-4- | |
| | yl)-1H-indol-1-yl)-2- | |
| | methylcyclopropyl)-1,2,4- | |
| | oxadiazol-5(4H)-one | |
| 41 | 3-((1S,2S)-1-(5-((S)-2,2- | 904.6 |
| | dimethyltetrahydro-2H-pyran-4- | |
| | yl)-2-((S)-2-(4-fluoro-3,5- | |
| | dimethylphenyl)-4,7,7-trimethyl-3- | |
| | (1-(1-methyl-1H-indazol-5-yl)-2- | |
| | oxo-1,2-dihydropyridin-3-yl)- | |
| | 4,5,6,7-tetrahydro-2H- | |
| | pyrazolo[4,3-c]pyridine-5- | |
| | carbonyl)-1H-indol-1-yl)-2- | |
| | methylcyclopropyl)-1,2,4- | |
| | oxadiazol-5(4H)-one | |
| 42 | 3-((1S,2S)-1-(2-((S)-2′-(4-fluoro- | 874.7 |
| | 3,5-dimethylphenyl)-4′-methyl-3′- | |
| | (1-(1-methyl-1H-indazol-5-yl)-2- | |
| | oxo-1,2-dihydropyridin-3-yl)- | |
| | 2′,4′,5′,6′- | |
| | tetrahydrospiro[cyclopropane-1,7′- | |
| | pyrazolo[4,3-c]pyridine]-5′- | |
| | carbonyl)-5-(tetrahydro-2H-pyran- | |
| | 4-yl)-1H-indol-1-yl)-2- | |
| | methylcyclopropyl)-1,2,4- | |
| | oxadiazol-5(4H)-one | |
| 43 | 3-((1S,2S)-1-(2-((S)-2-(4-fluoro- | 902.7 |
| | 3,5-dimethylphenyl)-4-methyl-3- | |
| | (2-oxo-1-(2- | |
| | (trifluoromethyl)imidazo[1,2- | |
| | a]pyridin-6-yl)-1,2- | |
| | dihydropyridin-3-yl)-4,5,6,7- | |
| | tetrahydro-2H-pyrazolo[4,3- | |
| | c]pyridine-5-carbonyl)-5- | |
| | (tetrahydro-2H-pyran-4-yl)-1H- | |
| | indol-1-yl)-2-methylcyclopropyl)- | |
| | 1,2,4-oxadiazol-5(4H)-one | |
| 44 | 3-((1S,2S)-1-(2-((S)-3-(1-(2- | 875.7 |
| | cyclopropyl-[1,2,4]triazolo[1,5- | |
| | a]pyridin-6-yl)-2-oxo-1,2- | |
| | dihydropyridin-3-yl)-2-(4-fluoro- | |
| | 3,5-dimethylphenyl)-4-methyl- | |
| | 4,5,6,7-tetrahydro-2H- | |
| | pyrazolo[4,3-c]pyridine-5- | |
| | carbonyl)-5-(tetrahydro-2H-pyran- | |
| | 4-yl)-1H-indol-1-yl)-2- | |
| | methylcyclopropyl)-1,2,4- | |
| | oxadiazol-5(4H)-one | |
| 45 | 3-((1S,2S)-1-(2-((S)-2-(4-fluoro- | 902.7 |
| | 3,5-dimethylphenyl)-4-methyl-3- | |
| | (2-oxo-1-(3- | |
| | (trifluoromethyl)imidazo[1,5- | |
| | a]pyridin-7-yl)-1,2- | |
| | dihydropyridin-3-yl)-4,5,6,7- | |
| | tetrahydro-2H-pyrazolo[4,3- | |
| | c]pyridine-5-carbonyl)-5- | |
| | (tetrahydro-2H-pyran-4-yl)-1H- | |
| | indol-1-yl)-2-methylcyclopropyl)- | |
| | 1,2,4-oxadiazol-5(4H)-one | |
| 46 | 3-((1S,2S)-1-(2-((S)-3-(1-(1- | 884.6 |
| | (difluoromethyl)-1H-indazol-5-yl)- | |
| | 2-oxo-1,2-dihydropyridin-3-yl)-2- | |
| | (4-fluoro-3,5-dimethylphenyl)-4- | |
| | methyl-4,5,6,7-tetrahydro-2H- | |
| | pyrazolo[4,3-c]pyridine-5- | |
| | carbonyl)-5-(tetrahydro-2H-pyran- | |
| | 4-yl)-1H-indol-1-yl)-2- | |
| | methylcyclopropyl)-1,2,4- | |
| | oxadiazol-5(4H)-one | |
| 47 | 3-((1S,2S)-1-(2-((S)-3-(1-(1- | 912.6 |
| | (difluoromethyl)-1H-indazol-5-yl)- | |
| | 2-oxo-1,2-dihydropyridin-3-yl)-2- | |
| | (4-fluoro-3,5-dimethylphenyl)-4- | |
| | methyl-4,5,6,7-tetrahydro-2H- | |
| | pyrazolo[4,3-c]pyridine-5- | |
| | carbonyl)-5-((S)-2,2- | |
| | dimethyltetrahydro-2H-pyran-4- | |
| | yl)-1H-indol-1-yl)-2- | |
| | methylcyclopropyl)-1,2,4- | |
| | oxadiazol-5(4H)-one | |
| 48 | 3-((1S,2S)-1-(5-((S)-2,2- | 837.7 |
| | dimethyltetrahydro-2H-pyran-4- | |
| | yl)-2-((S)-2-(4-fluoro-3,5- | |
| | dimethylphenyl)-4-methyl-3-(2′- | |
| | methyl-2-oxo-2H-[1,4′-bipyridin]- | |
| | 3-yl)-4,5,6,7-tetrahydro-2H- | |
| | pyrazolo[4,3-c]pyridine-5- | |
| | carbonyl)-1H-indol-1-yl)-2- | |
| | methylcyclopropyl)-1,2,4- | |
| | oxadiazol-5(4H)-one | |
| 49 | 3-((1S,2S)-1-(5-((S)-2,2- | 862.8 |
| | dimethyltetrahydro-2H-pyran-4- | |
| | yl)-2-((S)-2-(4-fluoro-3,5- | |
| | dimethylphenyl)-3-(1- | |
| | (imidazo[1,5-a]pyridin-6-yl)-2- | |
| | oxo-1,2-dihydropyridin-3-yl)-4- | |
| | methyl-4,5,6,7-tetrahydro-2H- | |
| | pyrazolo[4,3-c]pyridine-5- | |
| | carbonyl)-1H-indol-1-yl)-2- | |
| | methylcyclopropyl)-1,2,4- | |
| | oxadiazol-5(4H)-one | |
| 50 | 3-((1S,2S)-1-(2-((S)-3′-(2′- | 877.6 |
| | cyclopropoxy-2-oxo-2H-[1,4′- | |
| | bipyridin]-3-yl)-2′-(4-fluoro-3,5- | |
| | dimethylphenyl)-4′-methyl- | |
| | 2′,4′,5′,6′- | |
| | tetrahydrospiro[cyclopropane-1,7′- | |
| | pyrazolo[4,3-c]pyridine]-5′- | |
| | carbonyl)-5-(tetrahydro-2H-pyran- | |
| | 4-yl)-1H-indol-1-yl)-2- | |
| | methylcyclopropyl)-1,2,4- | |
| | oxadiazol-5(4H)-one | |
| 51 | 3-((1S,2S)-1-(2-((S)-3-(2′- | 907.8 |
| | cyclopropoxy-2-oxo-2H-[1,4′- | |
| | bipyridin]-3-yl)-2-(4-fluoro-3,5- | |
| | dimethylphenyl)-4,7,7-trimethyl- | |
| | 4,5,6,7-tetrahydro-2H- | |
| | pyrazolo[4,3-c]pyridine-5- | |
| | carbonyl)-5-((S)-2,2- | |
| | dimethyltetrahydro-2H-pyran-4- | |
| | yl)-1H-indol-1-yl)-2- | |
| | methylcyclopropyl)-1,2,4- | |
| | oxadiazol-5(4H)-one | |
| 52 | 3-((1S,2S)-1-(2-((S)-3-(2′- | 879.6 |
| | cyclopropoxy-2-oxo-2H-[1,4′- | |
| | bipyridin]-3-yl)-2-(4-fluoro-3,5- | |
| | dimethylphenyl)-4,7,7-trimethyl- | |
| | 4,5,6,7-tetrahydro-2H- | |
| | pyrazolo[4,3-c]pyridine-5- | |
| | carbonyl)-5-(tetrahydro-2H-pyran- | |
| | 4-yl)-1H-indol-1-yl)-2- | |
| | methylcyclopropyl)-1,2,4- | |
| | oxadiazol-5(4H)-one | |
| 53 | 3-((1S,2S)-1-(2-((S)-3′-(2′- | 905.6 |
| | cyclopropoxy-2-oxo-2H-[1,4′- | |
| | bipyridin]-3-yl)-2′-(4-fluoro-3,5- | |
| | dimethylphenyl)-4′-methyl- | |
| | 2′,4′,5′,6′- | |
| | tetrahydrospiro[cyclopropane-1,7′- | |
| | pyrazolo[4,3-c]pyridine]-5′- | |
| | carbonyl)-5-((S)-2,2- | |
| | dimethyltetrahydro-2H-pyran-4- | |
| | yl)-1H-indol-1-yl)-2- | |
| | methylcyclopropyl)-1,2,4- | |
| | oxadiazol-5(4H)-one | |
| 54 | 3-((1S,2S)-1-(2-((S)-3-(2′- | 879.6 |
| | cyclopropoxy-2-oxo-2H-[1,4′- | |
| | bipyridin]-3-yl)-2-(4-fluoro-3,5- | |
| | dimethylphenyl)-4-methyl-4,5,6,7- | |
| | tetrahydro-2H-pyrazolo[4,3- | |
| | c]pyridine-5-carbonyl)-5-((S)-2,2- | |
| | dimethyltetrahydro-2H-pyran-4- | |
| | yl)-1H-indol-1-yl)-2- | |
| | methylcyclopropyl)-1,2,4- | |
| | oxadiazol-5(4H)-one | |
| 55 | 3-((1S,2S)-1-(2-((S)-3-(2′- | 851.6 |
| | cyclopropoxy-2-oxo-2H-[1,4′- | |
| | bipyridin]-3-yl)-2-(4-fluoro-3,5- | |
| | dimethylphenyl)-4-methyl-4,5,6,7- | |
| | tetrahydro-2H-pyrazolo[4,3- | |
| | c]pyridine-5-carbonyl)-5- | |
| | (tetrahydro-2H-pyran-4-yl)-1H- | |
| | indol-1-yl)-2-methylcyclopropyl)- | |
| | 1,2,4-oxadiazol-5(4H)-one | |
| 56 | 3-((1S,2S)-1-(2-((S)-2-(4-fluoro- | 845.6 |
| | 3,5-dimethylphenyl)-3-(1- | |
| | (isoquinolin-6-yl)-2-oxo-1,2- | |
| | dihydropyridin-3-yl)-4-methyl- | |
| | 4,5,6,7-tetrahydro-2H- | |
| | pyrazolo[4,3-c]pyridine-5- | |
| | carbonyl)-5-(tetrahydro-2H-pyran- | |
| | 4-yl)-1H-indol-1-yl)-2- | |
| | methylcyclopropyl)-1,2,4- | |
| | oxadiazol-5(4H)-one | |
| 57 | 3-((1S,2S)-1-(2-((S)-3-(1-(4- | 927.6 |
| | (diethylphosphoryl)-3- | |
| | (methylamino)phenyl)-2-oxo-1,2- | |
| | dihydropyridin-3-yl)-2-(4-fluoro- | |
| | 3,5-dimethylphenyl)-4-methyl- | |
| | 4,5,6,7-tetrahydro-2H- | |
| | pyrazolo[4,3-c]pyridine-5- | |
| | carbonyl)-5-(tetrahydro-2H-pyran- | |
| | 4-yl)-1H-indol-1-yl)-2- | |
| | methylcyclopropyl)-1,2,4- | |
| | oxadiazol-5(4H)-one | |
| 58 | 3-((1S,2S)-1-(2-((S)-2-(4-fluoro- | 879.6 |
| | 3,5-dimethylphenyl)-4-methyl-3- | |
| | (1-(4-morpholinophenyl)-2-oxo- | |
| | 1,2-dihydropyridin-3-yl)-4,5,6,7- | |
| | tetrahydro-2H-pyrazolo[4,3- | |
| | c]pyridine-5-carbonyl)-5- | |
| | (tetrahydro-2H-pyran-4-yl)-1H- | |
| | indol-1-yl)-2-methylcyclopropyl)- | |
| | 1,2,4-oxadiazol-5(4H)-one | |
| 59 | 3-((1S,2S)-1-(2-((S)-7,7-difluoro- | 912.2 |
| | 2-(4-fluoro-3,5-dimethylphenyl)-4- | |
| | methyl-3-(1-(1-methyl-1H- | |
| | indazol-5-yl)-2-oxo-1,2- | |
| | dihydropyridin-3-yl)-4,5,6,7- | |
| | tetrahydro-2H-pyrazolo[4,3- | |
| | c]pyridine-5-carbonyl)-5-((S)-2,2- | |
| | dimethyltetrahydro-2H-pyran-4- | |
| | yl)-1H-indol-1-yl)-2- | |
| | methylcyclopropyl)-1,2,4- | |
| | oxadiazol-5(4H)-one | |
| 60 | 3-((1S,2S)-1-(5-((S)-2,2- | 905.8 |
| | dimethyltetrahydro-2H-pyran-4- | |
| | yl)-2-((S)-2-(4-fluoro-3,5- | |
| | dimethylphenyl)-4,7,7-trimethyl-3- | |
| | (1-(1-methyl-1H-indazol-5-yl)-6- | |
| | oxo-1,6-dihydropyrimidin-5-yl)- | |
| | 4,5,6,7-tetrahydro-2H- | |
| | pyrazolo[4,3-c]pyridine-5- | |
| | carbonyl)-1H-indol-1-yl)-2- | |
| | methylcyclopropyl)-1,2,4- | |
| | oxadiazol-5(4H)-one | |
| 61 | 3-((1S,2S)-1-(5-((S)-2,2- | 890.4 |
| | dimethyltetrahydro-2H-pyran-4- | |
| | yl)-2-((S)-2-(4-fluoro-3,5- | |
| | dimethylphenyl)-4-methyl-3-(1-(1- | |
| | methyl-1H-indazol-5-yl)-2-oxo- | |
| | 1,2-dihydropyridin-3-yl)- | |
| | 2,4,5,6,7,8- | |
| | hexahydropyrazolo[4,3-c]azepine- | |
| | 5-carbonyl)-1H-indol-1-yl)-2- | |
| | methylcyclopropyl)-1,2,4- | |
| | oxadiazol-5(4H)-one | |
| 187 | 3-((1S,2S)-1-(5-((S)-2,2- | 887.4 |
| | dimethyltetrahydro-2H-pyran-4- | |
| | yl)-2-((4S)-2-(4-fluoro-3,5- | |
| | dimethylphenyl)-4-methyl-3-(1-(2- | |
| | methylquinolin-6-yl)-2-oxo-1,2- | |
| | dihydropyridin-3-yl)-4,5,6,7- | |
| | tetrahydro-2H-pyrazolo[4,3- | |
| | c]pyridine-5-carbonyl)-1H-indol- | |
| | 1-yl)-2-methylcyclopropyl)-1,2,4- | |
| | oxadiazol-5(4H)-one | |
| 188 | 3-((1S,2S)-1-(2-((4S)-2-(4-fluoro- | 874.6 |
| | 3,5-dimethylphenyl)-4-methyl-3- | |
| | (1-(2-methylquinolin-6-yl)-2-oxo- | |
| | 1,2-dihydropyridin-3-yl)-4,5,6,7- | |
| | tetrahydro-2H-pyrazolo[4,3- | |
| | c]pyridine-5-carbonyl)-5-((S)-2- | |
| | methylmorpholino)-1H-indol-1- | |
| | yl)-2-methylcyclopropyl)-1,2,4- | |
| | oxadiazol-5(4H)-one | |
| 189 | 3-((1S,2S)-1-(5-((S)-2,2- | 888.4 |
| | dimethyltetrahydro-2H-pyran-4- | |
| | yl)-2-((4S)-2-(4-fluoro-3,5- | |
| | dimethylphenyl)-4-methyl-3-(1-(2- | |
| | methylquinolin-6-yl)-6-oxo-1,6- | |
| | dihydropyrimidin-5-yl)-4,5,6,7- | |
| | tetrahydro-2H-pyrazolo[4,3- | |
| | c]pyridine-5-carbonyl)-1H-indol- | |
| | 1-yl)-2-methylcyclopropyl)-1,2,4- | |
| | oxadiazol-5(4H)-one | |
| 190 | 3-[(1S,2S)-1-[5-[(4S)-2,2- | 909.4 |
| | dimethyltetrahydropyran-4-yl]-2- | |
| | [(4S)-2-(4-fluoro-3,5-dimethyl- | |
| | phenyl)-3-[1-(4-fluoro-1-methyl- | |
| | indazol-5-yl)-6-oxo-pyrimidin-5- | |
| | yl]-4-methyl-4,6,7,8- | |
| | tetrahydropyrazolo[4,3-c]azepin-1- | |
| | ium-5-carbonyl]indol-1-yl]-2- | |
| | methyl-cyclopropyl]-4H-1,2,4- | |
| | oxadiazol-5-one formate | |
| 191 | 3-[(1S,2S)-1-[5-[(4S)-2,2- | 901.4 |
| | dimethyltetrahydropyran-4-yl]-2- | |
| | [(4S)-2-(4-fluoro-3,5-dimethyl- | |
| | phenyl)-4-methyl-3-[1-(1- | |
| | methylisoquinolin-2-ium-6-yl)-2- | |
| | oxo-3-pyridyl]-4,6,7,8- | |
| | tetrahydropyrazolo[4,3-c]azepine- | |
| | 5-carbonyl]indol-1-yl]-2-methyl- | |
| | cyclopropyl]-4H-1,2,4-oxadiazol- | |
| | 5-one formate | |
| 192 | 3-[(1S,2S)-1-[2-[(4S)-3-[1-[2- | 893.4 |
| | (cyclopropoxy)pyridin-1-ium-4- | |
| | yl]-2-oxo-3-pyridyl]-2-(4-fluoro- | |
| | 3,5-dimethyl-phenyl)-4-methyl- | |
| | 4,6,7,8-tetrahydropyrazolo[4,3- | |
| | cJazepine-5-carbonyl]-5-[(4S)-2,2- | |
| | dimethyltetrahydropyran-4- | |
| | yl]indol-1-yl]-2-methyl- | |
| | cyclopropyl]-4H-1,2,4-oxadiazol- | |
| | 5-one formate | |
| 193 | 3-[(1S,2S)-1-[5-[(4S)-2,2- | 908.5 |
| | dimethyltetrahydropyran-4-yl]-2- | |
| | [(4S)-2-(4-fluoro-3,5-dimethyl- | |
| | phenyl)-3-[1-(4-fluoro-1-methyl- | |
| | indazol-5-yl)-2-oxo-3-pyridyl]-4- | |
| | methyl-4,6,7,8- | |
| | tetrahydropyrazolo[4,3-c]azepin-1- | |
| | ium-5-carbonyl]indol-1-yl]-2- | |
| | methyl-cyclopropyl]-4H-1,2,4- | |
| | oxadiazol-5-one formate | |
| 194 | 3-[(1S,2S)-1-[2-[(4S)-2-(4-fluoro- | 906.4 |
| | 3,5-dimethyl-phenyl)-3-[1-(4- | |
| | fluoro-1-methyl-indazol-2-ium-5- | |
| | yl)-2-oxo-3-pyridyl]-4-methyl- | |
| | spiro[4,6-dihydropyrazolo[4,3- | |
| | c]pyridine-7,1′-cyclobutane]-5- | |
| | carbonyl]-5-tetrahydropyran-4-yl- | |
| | indol-1-yl]-2-methyl-cyclopropyl]- | |
| | 4H-1,2,4-oxadiazol-5-one;2,2,2- | |
| | trifluoroacetate | |
| 195 | 3-[(1S,2S)-1-[5-[(4S)-2,2- | 905.5 |
| | dimethyltetrahydropyran-4-yl]-2- | |
| | [(4S)-2-(4-fluoro-3,5-dimethyl- | |
| | phenyl)-3-[1-(5-fluoro-2-methyl-6- | |
| | quinolyl)-2-oxo-3-pyridyl]-4- | |
| | methyl-6,7-dihydro-4H- | |
| | pyrazolo[4,3-c]pyridine-5- | |
| | carbonyl]indol-1-yl]-2-methyl- | |
| | cyclopropyl]-4H-1,2,4-oxadiazol- | |
| | 5-one | |
| 196 | 3-[(1S,2S)-1-[5-[(4S)-2,2- | 1007.4 |
| | dimethyltetrahydropyran-4-yl]-2- | |
| | [(4S)-2-(4-fluoro-3,5-dimethyl- | |
| | phenyl)-3-[1-(4-fluoro-1-methyl- | |
| | indazol-5-yl)-2-oxo-3-pyridyl]-4- | |
| | methyl-1′-(2,2,2- | |
| | trifluoroethyl)spiro[4,6- | |
| | dihydropyrazolo[4,3-c]pyridine- | |
| | 7,3′-azetidin-1-ium]-5- | |
| | carbonyl]indol-1-yl]-2-methyl- | |
| | cyclopropyl]-4H-1,2,4-oxadiazol- | |
| | 5-one;2,2,2-trifluoroacetate | |
| 197 | 3-[(1S,2S)-1-[5-[(4S)-2,2- | 905.3 |
| | dimethyltetrahydropyran-4-yl]-2- | |
| | [(4S)-2-(4-fluoro-3,5-dimethyl- | |
| | phenyl)-3-[1-(5-fluoro-1-methyl- | |
| | isoquinolin-2-ium-6-yl)-2-oxo-3- | |
| | pyridyl]-4-methyl-6,7-dihydro-4H- | |
| | pyrazolo[4,3-c]pyridine-5- | |
| | carbonyl]indol-1-yl]-2-methyl- | |
| | cyclopropyl]-4H-1,2,4-oxadiazol- | |
| | 5-one;2,2,2-trifluoroacetate | |
| 198 | 3-((1S,2S)-1-(5-((S)-2,2- | 934.4 |
| | dimethyltetrahydro-2H-pyran-4- | |
| | yl)-2-((4S)-3-(1-(5-fluoro-1- | |
| | methylisoquinolin-6-yl)-2-oxo-1,2- | |
| | dihydropyridin-3-yl)-2-(4-fluoro- | |
| | 3,5-dimethylphenyl)-4-methyl- | |
| | 4,5,6,7-tetrahydro-2H- | |
| | pyrazolo[4,3-c]pyridine-5- | |
| | carbonyl)-1H-indol-1-yl)-2- | |
| | methylcyclopropyl)-1,2,4- | |
| | oxadiazol-5(4H)-one | |

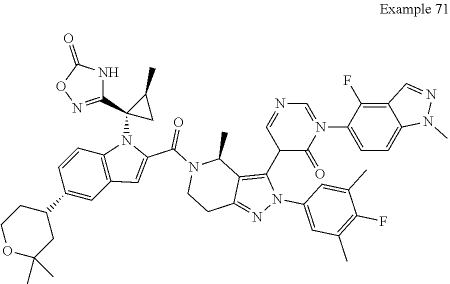

### 3-((1S,2S)-1-(5-((S)-2,2-dimethyltetrahydro-2H-pyran-4-yl)-2-((S)-3-(1-(4-fluoro-1-methyl-1H-indazol-5-yl)-6-oxo-1,6-dihydropyrimidin-5-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one

### Step 1: 3-((4S)-2-(4-fluoro-3,5-dimethylphenyl)-4-methyl-4,5,6,7-tetrahydro-2H-pyrazolo[4,3-c]pyridin-3-yl)pyridin-2(1H)-one

This step was performed in a similar fashion as in Step 1 as described for Example 26 to provide the title compound (without reprotection). MS (ESI) m/z:353.3 [M+H+].

### Step 2: 3-((1S,2S)-1-(5-((S)-2,2-dimethyltetrahydro-2H-pyran-4-yl)-2-((4S)-2-(4-fluoro-3,5-dimethylphenyl)-4-methyl-3-(6-oxo-1,6-dihydropyrimidin-5-yl)-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one

This step was performed in a similar fashion as in Step 3 described for Example 26 to provide the title compound. MS (ESI) m/z: 747.4 [M+H+]

### Step 4: 3-((1S,2S)-1-(5-((S)-2,2-dimethyltetrahydro-2H-pyran-4-yl)-2-((4S)-3-(1-(4-fluoro-1-methyl-1H-indazol-5-yl)-6-oxo-1,6-dihydropyrimidin-5-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one

A vial was charged with 3-((1S,2S)-1-(5-((S)-2,2-dimethyltetrahydro-2H-pyran-4-yl)-2-((4S)-2-(4-fluoro-3,5-dimethylphenyl)-4-methyl-3-(6-oxo-1,6-dihydropyrimidin-5-yl)-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one (172 mg, 230 mol) and 2-chloro-1-methyl-pyridin-1-ium iodide (Mukaiyama's reagent, 103 mg, 403 mol) and DCM (1 mL) followed by Hunig's base (100 μL, 576 mol). The mixture was allowed to stir at room temperatrure for 1 hour. The mixture was concentrated under reduced pressure and resuspended in DCE (1 mL) and 4-fluoro-1-methyl-1H-indazol-5-amine (57.1 mg, 345 mol) followed by Hunig's base (100 L, 576 mol) was added and the mixture was heated to 80° C. for 2 days. The reaction mixture was purified by silica gel chromatography (0% to 100% EtOAc/hexanes) to provide the title compound. MS (ESI) m/z: 895.6 [M+H+]. ¹H NMR (400 MHz, DMSO-d6) δ ppm 0.97-1.21 (m, 5H) 1.21-1.43 (m, 6H) 1.45-1.56 (m, 2H) 1.57-1.88 (m, 5H) 1.90-2.30 (m, 6H) 1.90-2.29 (m, 1H) 2.63-2.94 (m, 1H) 3.03(br t, J=11.88 Hz, 1H) 3.10-3.26 (m, 1H) 3.40-3.80 (m, 3H) 3.99-4.19 (m, 3H) 4.23-4.88 (m, 1H) 5.18-5.91 (m, 1H) 6.63-6.99 (m, 1H) 7.13 (br s, 2H) 7.25 (br d, J=8.25 Hz, 1H) 7.30-7.43 (m, 1H) 7.44-7.56 (m, 1H) 7.61 (br s, 1H) 7.66-7.79 (m, 1H) 7.89-8.25 (m, 1H) 8.35 (s, 1H) 8.46-8.77 (m, 1H) 11.59-12.36 (m, 1H).

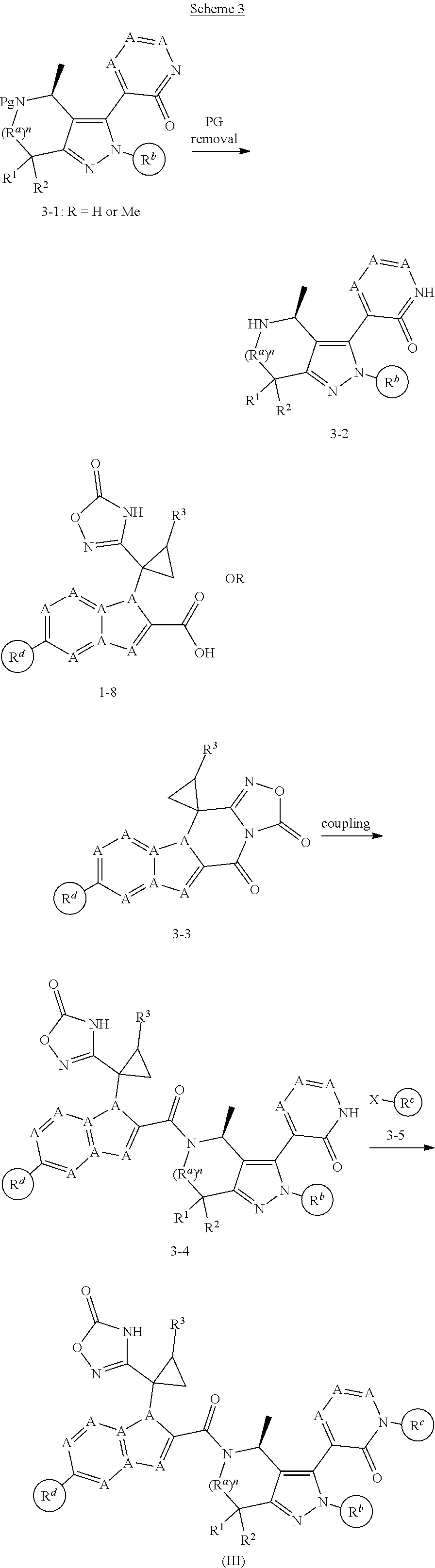

Compounds of formula (III) are prepared from intermediate 3-1 by removal of the protecting groups and then coupling of 3-2 with carboxylic acid 1-8 or heterocycle 3-3 to afford adduct 3-4. Subsequently, 3-4 is coupled with halides, boronic acids, boronates, or sulfonates or other suitably functionalized partners (3-5) via transition metal catalysis (e.g., Pd—or Cu-catalysis) or alkylation (e.g., basic) conditions to provide compounds of formula (III).

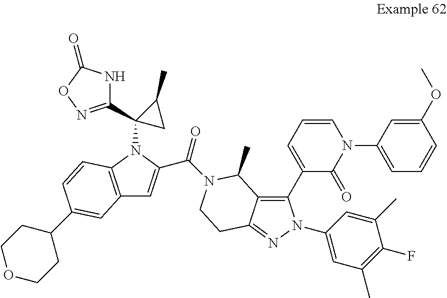 [SMILES: COc1cccc(-n2cccc(-c3c4c(nn3-c3cc(C)c(F)c(C)c3)CCN(C(=O)c3cc5cc(C6CCOCC6)ccc5n3[C@@]3(c5noc(=O)[nH]5)C[C@@H]3C)[C@H]4C)c2=O)c1]

### 3-((1S,2S)-1-(2-((S)-2-(4-fluoro-3,5-dimethylphenyl)-3-(1-(3-methoxyphenyl)-2-oxo-1,2-dihydropyridin-3-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one (Scheme 3)

### Step 1: (S)-3-(2-(4-fluoro-3,5-dimethylphenyl)-4-methyl-4,5,6,7-tetrahydro-2H-pyrazolo[4,3-c]pyridin-3-yl)pyridin-2(1H)-one

A mixture of tert-butyl (4S)-2-(4-fluoro-3,5-dimethylphenyl)-3-(2-methoxypyridin-3-yl)-4-methyl-2,4,6,7-tetrahydro-5H-pyrazolo[4,3-c]pyridine-5-carboxylate (304 mg, 0.652 mmol) in acetic acid (2.2 mL) was treated with HBr (33% wt in acetic acid, 800 mg, 0.54 mL, 3.26 mmol) and stirred at 60° C. for 20 min. The reaction was then quenched with methanol and concentrated under reduced pressure to afford the title compound as the HBr salt, which was used further without purification. MS (ESI) m/z: 353.4 [M+H]+.

### Step 2: 3-((1S,2S)-1-(2-((S)-2-(4-fluoro-3,5-dimethylphenyl)-4-methyl-3-(2-oxo-1,2-dihydropyridin-3-yl)-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one

A mixture of (S)-3-(2-(4-fluoro-3,5-dimethylphenyl)-4-methyl-4,5,6,7-tetrahydro-2H-pyrazolo[4,3-c]pyridin-3-yl)pyridin-2(1H)-one (HBr salt, 300 mg, 0.692 mmol), (1S,2S)-2-methyl-8′-(tetrahydro-2H-pyran-4-yl)-3′H,5′H-spiro[cyclopropane-1,12′-[1,2,4]oxadiazolo[4′,3′:4,5]pyrazino[1,2-a]indole]-3′,5′-dione (379 mg, 1.04 mmol) in DMF (2.8 mL) was treated with DIPEA (358 mg, 0.48 mL, 2.77 mmol) and stirred at rt overnight. At this point, the mixture was treated with additional (1S,2S)-2-methyl-8′-(tetrahydro-2H-pyran-4-yl)-3′H,5′H-spiro[cyclopropane-1,12′-[1,2,4]oxadiazolo[4′,3′:4,5]pyrazino[1,2-a]indole]-3′,5′-dione (100 mg, 0.274 mmol) and stirred at rt for 5 h. The reaction mixture was then directly purified via C18 reverse phase chromatography (10-100% MeCN/water+0.1% formic acid modifier) to afford the title compound as a solid. MS (ESI) m/z: 718.6 [M+H]+.

### Step 3: 3-((1S,2S)-1-(2-((S)-2-(4-fluoro-3,5-dimethylphenyl)-3-(1-(3-methoxyphenyl)-2-oxo-1,2-dihydropyridin-3-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one

A mixture of 3-((1S,2S)-1-(2-((4S)-2-(4-fluoro-3,5-dimethylphenyl)-4-methyl-3-(2-oxo-1,2-dihydropyridin-3-yl)-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one (45 mg, 0.063 mmol), 1-cyclopropyl-5-iodo-1H-indazole (89 mg, 0.31 mmol), copper(I) iodide (3.0 mg, 0.016 mmol) and potassium phosphate tribasic (40 mg, 0.19 mmol) in dioxane (0.31 mL) was sparged with argon for 5 min. The mixture was then treated with N,N′-dimethylethylenediamine (2.8 mg, 3.4 μL, 0.031 mmol) and stirred under argon at 110° C. for 105 min. The mixture was then cooled down, concentrated, acidified with formic acid and purified via C18 reverse-phase column chromatography (10-100% MeCN/water+0.1% formic acid modifier) to afford the title compound as a solid. ¹H NMR (500 MHz, CD₃CN) δ 7.61-7.39 (m, 4H), 7.34-7.05 (m, 3H), 7.06-6.86 (m, 3H), 6.85-6.40 (m, 2H), 6.39-6.09 (m, 1H), 5.87-5.21 (m, 1H), 4.86-4.32 (m, 1H), 4.03-3.96 (m, 2H), 3.83-3.72 (m, 3H), 3.65-3.45 (m, 3H), 3.20-2.80 (m, 3H), 2.26-2.19 (m, 6H), 1.83-1.62 (m, 6H), 1.59-1.37 (m, 4H), 1.17-0.94 (m, 3H). MS (ESI) m/z: 824.6 [M+H]+. The following examples in table 3 were prepared according to scheme 3 using the procedures outlined in the synthesis of Example 62.

TABLE 3

| | | | MS |
|---|---|---|---|
| Ex. | Structure | Name | (M + 1) |
| 63 | 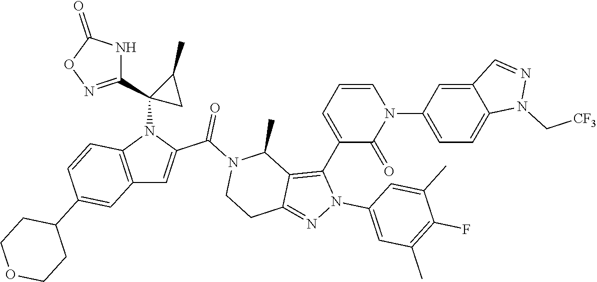 [SMILES: Cc1cc(-n2nc3c(c2-c2cccn(-c4ccc5c(cnn5CC(F)(F)F)c4)c2=O)[C@H](C)N(C(=O)c2cc4cc(C5CCOCC5)ccc4n2[C@@]2(c4noc(=O)[nH]4)C[C@@H]2C)CC3)cc(C)c1F] | 3-((1S,2S)-1-(2-((S)-2-(4- fluoro-3,5-dimethylphenyl)-4- methyl-3-(2-oxo-1-(1-(2,2,2- trifluoroethyl)-1H-indazol-5- yl)-1,2-dihydropyridin-3-yl)- 4,5,6,7-tetrahydro-2H- pyrazolo[ 4,3-c] pyridine-5- carbonyl)-5-(tetrahydro-2H- pyran-4-yl)-1H-indol-1-yl)-2- methylcyclopropyl)-1,2,4- oxadiazol-5(4H)-one | 916.6 |
| 64 | 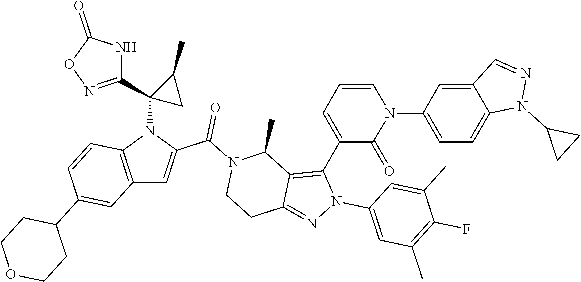 [SMILES: Cc1cc(-n2nc3c(c2-c2cccn(-c4ccc5c(cnn5C5CC5)c4)c2=O)[C@H](C)N(C(=O)c2cc4cc(C5CCOCC5)ccc4n2[C@@]2(c4noc(=O)[nH]4)C[C@@H]2C)CC3)cc(C)c1F] | 3-((1S,2S)-1-(2-((S)-3-(1-(1- cyclopropyl-1H-indazol-5-yl)- 2-oxo-1,2-dihydropyridin-3- yl)-2-(4-fluoro-3,5- dimethylphenyl)-4-methyl- 4,5,6,7-tetrahydro-2H- pyrazolo[4,3-c]pyridine-5- carbonyl)-5-(tetrahydro-2H- pyran-4-yl)-1H-indol-1-yl)-2- methylcyclopropyl)-1,2,4- oxadiazol-5(4H)-one | 874.6 |

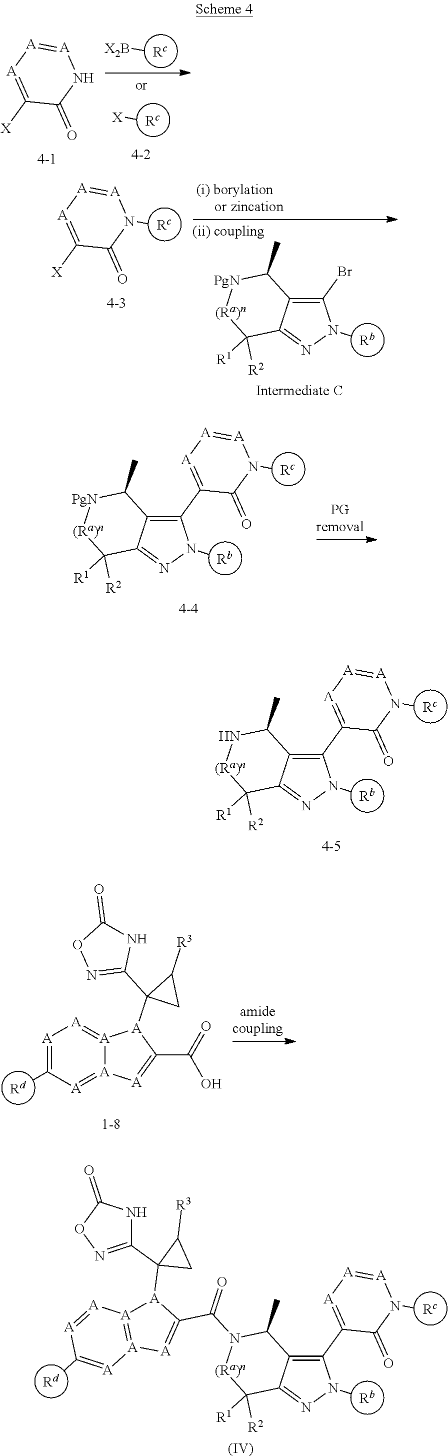

Compounds of formula (IV) are prepared from intermediate 4-1 by coupling with aryl or heteroaryl boronic acid or other boronate partners or halides (4-2) via transition metal catalysis (e.g., Cu—or Pd-catalysis) or basic conditions. Halide 4-3 is converted to a boronate or zincate and then coupled with intermediate C to provide 4-4 via transition metal catalyzed cross-coupling (e.g., Pd-catalysis). Removal of the protecting group (e.g., acidic conditions) of 4-4 provides amine 4-5 that is then coupled with carboxylic acid 1-8 to provide compounds of formula (IV).

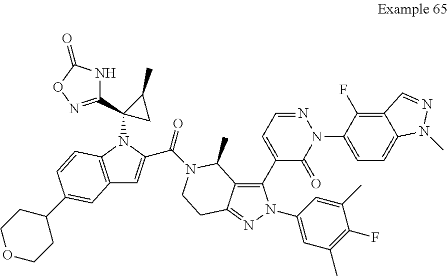 [SMILES: Cc1cc(-n2nc3c(c2-c2ccnn(-c4ccc5c(cnn5C)c4F)c2=O)[C@H](C)N(C(=O)c2cc4cc(C5CCOCC5)ccc4n2[C@@]2(c4noc(=O)[nH]4)C[C@@H]2C)CC3)cc(C)c1F]

### 3-((1S,2S)-1-(2-((S)-3-(2-(4-fluoro-1-methyl-1H-indazol-5-yl)-3-oxo-2,3-dihydropyridazin-4-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one (Scheme 4)

### Step 1: 4-chloro-2-(4-fluoro-1-methyl-1H-indazol-5-yl)pyridazin-3(2H)-one

A mixture of 4-chloropyridazin-3-ol (100 mg, 0.766 mmol), (4-fluoro-1-methyl-1H-indazol-5-yl)boronic acid (223 mg, 1.15 mmol), copper(I) acetate (18.8 mg, 0.153 mmol), and triethylamine (77.5 mg, 766 mmol) in CH₂Cl₂ (5.0 mL) was stirred at 23° C. for 16 hour under an oxygen atmosphere. Then, the mixture was filtered through Celite® and washed with CH₂Cl₂ (2×10 mL). The filtrate was concentrated under reduced pressure, then purified by SiO₂ chromatography (acetone/CH₂Cl₂) to afford the title compound. MS (ESI) m/z: 279.2 [M+H+].

### Step 2: tert-butyl (S)-3-(2-(4-fluoro-1-methyl-1H-indazol-5-yl)-3-oxo-2,3-dihydropyridazin-4-yl)-2-(4-fluoro-3,5-dimethylphenyl)-4-methyl-2,4,6,7-tetrahydro-5H-pyrazolo[4,3-c]pyridine-5-carboxylate

To a mixture of 4-chloro-2-(4-fluoro-1-methyl-1H-indazol-5-yl)pyridazin-3(2H)-one (110 mg, 0.395 mmol) and potassium acetate (116 mg, 1.18 mmol) in dioxane (4.0 mL) was added bis(pinacolato)diborane (130 mg, 0.513 mmol). Argon was bubbled through the mixture for 10 min then [1,1′-bis(diphenylphosphino)ferrocene]dichloropalladium(II) CH₂Cl₂ complex (64.5 mg, 78.9 mol) was added, and argon was again bubbled through the mixture for 5 min. The reaction mixture was stirred at 110° C. for 3 h, then cooled to rt. To the mixture was added tert-butyl (S)-3-bromo-2-(4-fluoro-3,5-dimethylphenyl)-4-methyl-2,4,6,7-tetrahydro-5H-pyrazolo[4,3-c]pyridine-5-carboxylate (99.5 mg, 227 mmol) and potassium phosphate, dibasic (148 mg, 851 mmol). Argon was bubbled through the mixture for 10 min. Then, [1,1′-bis(diphenylphosphino)ferrocene]dichloropalladium(II) CH₂Cl₂ complex (46.3 mg, 56.7 mmol) was added and then argon was bubbled through the mixture for 5 min. The reaction mixture was stirred at 110° C. for 2 h, then cooled to rt. The reaction mixture was filtered through Celite® and washed with DCM (3×15 mL). The filtrate was concentrated under reduced pressure then purified by prep-HPLC (5 to 100% MeCN/H₂O+0.1% formic acid) to provide the title compound. MS (ESI) m/z: 602.4 [M+H+].

### Step 3: (S)-2-(4-fluoro-1-methyl-1H-indazol-5-yl)-4-(2-(4-fluoro-3,5-dimethylphenyl)-4-methyl-4,5,6,7-tetrahydro-2H-pyrazolo[4,3-c]pyridin-3-yl)pyrid9azin-3(2H)-one

A mixture of tert-butyl (S)-3-(2-(4-fluoro-1-methyl-1H-indazol-5-yl)-3-oxo-2,3-dihydropyridazin-4-yl)-2-(4-fluoro-3,5-dimethylphenyl)-4-methyl-2,4,6,7-tetrahydro-5H-pyrazolo[4,3-c]pyridine-5-carboxylate (135 mg, 0.224 mmol) and 4M HCl/Dioxane (0.56 mL, 2.24 mmol) was stirred at 22° C. for 2 h. Then, Et₂O (10 mL) was added to mixture. The resulting solid precipitate was filtered and washed with Et₂O (2×5 mL) to provide title compounds as a mixture. MS (ESI) m/z 502.3 [M+H]+.

### Step 4: 3-((1S,2S)-1-(2-((S)-3-(2-(4-fluoro-1-methyl-1H-indazol-5-yl)-3-oxo-2,3-dihydropyridazin-4-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one

This step was performed in a similar fashion as was described in example 1 to afford the title compound. ¹H NMR (400 MHz, methanol-d4) δ: 7.87-8.16 (2H, m), 7.43-7.53 (3H, m), 7.22-7.37 (2H, m), 7.09-7.16 (2H, m), 7.02-704 (1H, m), 6.84-6.87 (1H, m), 5.66-5.92 (1H, m), 4.38-4.74 (1H, m), 4.06-4.12 (5H, m), 3.47-3.64 (3H, m), 2.85-3.20 (3H, m), 2.24-2.26 (6H, m), 1.78 (5H, br s), 1.61-1.63 (1H, m), 1.54-1.56 (2H, m), 1.43-1.45 (2H, m), 1.18-1.32 (2H, m), 1.00-1.01 (1H, m). MS (ESI) m/z: 867.5 [M+H]+. The following examples in table 4 were prepared according to scheme 4 using the procedures outlined in the synthesis of example 65.

TABLE 4

| | | | MS |
|---|---|---|---|
| Ex. | Structure | Name | (M + 1) |
| 66 | 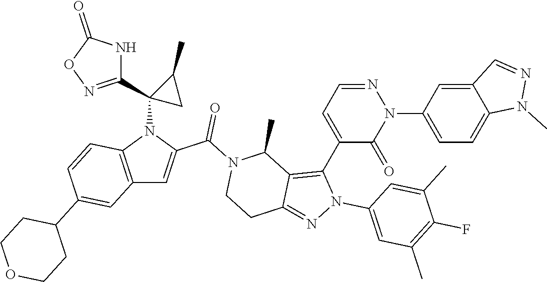 [SMILES: Cc1cc(-n2nc3c(c2-c2ccnn(-c4ccc5c(cnn5C)c4)c2=O)[C@H](C)N(C(=O)c2cc4cc(C5CCOCC5)ccc4n2[C@@]2(c4noc(=O)[nH]4)C[C@@H]2C)CC3)cc(C)c1F] | 3-((1S,2S)-1-(2-((S)-2-(4- fluoro-3,5-dimethylphenyl)-4- methyl-3-(2-(1-methyl-1H- indazol-5-yl)-3-oxo-2,3- dihydropyridazin-4-yl)-4,5,6,7- tetrahydro-2H-pyrazolo[4,3- c]pyridine-5-carbonyl)-5- (tetrahydro-2H-pyran-4-yl)-1H- indol-1-yl)-2- methylcyclopropyl)-1,2,4- oxadiazol-5(4H)-one | 849.6 |
| 67 | 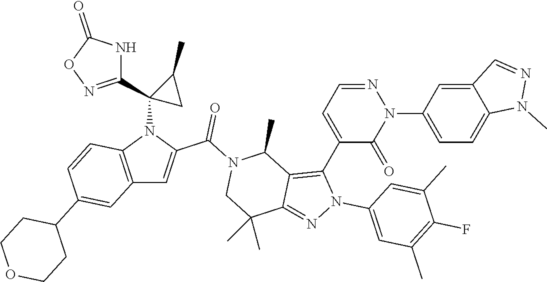 [SMILES: Cc1cc(-n2nc3c(c2-c2ccnn(-c4ccc5c(cnn5C)c4)c2=O)[C@H](C)N(C(=O)c2cc4cc(C5CCOCC5)ccc4n2[C@@]2(c4noc(=O)[nH]4)C[C@@H]2C)CC3(C)C)cc(C)c1F] | 3-((1S,2S)-1-(2-((S)-2-(4- fluoro-3,5-dimethylphenyl)- 4,7,7-trimethyl-3-(2-(1-methyl- 1H-indazol-5-yl)-3-oxo-2,3- dihydropyridazin-4-yl)-4,5,6,7- tetrahydro-2H-pyrazolo[4,3- c]pyridine-5-carbonyl)-5- (tetrahydro-2H-pyran-4-yl)-1H- indol-1-yl)-2- methylcyclopropyl)-1,2,4- oxadiazol-5(4H)-one | 877.5 |
| 68 | 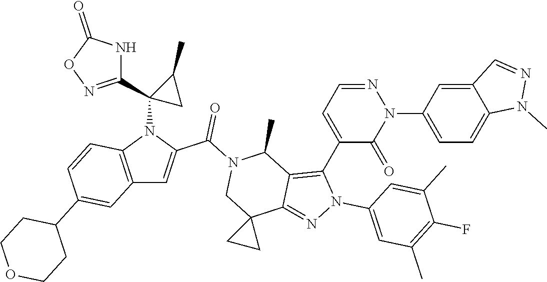 [SMILES: Cc1cc(-n2nc3c(c2-c2ccnn(-c4ccc5c(cnn5C)c4)c2=O)[C@H](C)N(C(=O)c2cc4cc(C5CCOCC5)ccc4n2[C@@]2(c4noc(=O)[nH]4)C[C@@H]2C)CC32CC2)cc(C)c1F] | 3-((1S,2S)-1-(2-((S)-2′-(4- fluoro-3,5-dimethylphenyl)-4′- methyl-3′-(2-(1-methyl-1H- indazol-5-yl)-3-oxo-2,3- dihydropyridazin-4-yl)- 2′,4′,5′,6′- tetrahydrospiro[cyclopropane- 1,7′-pyrazolo[4,3-c]pyridine]- 5′-carbonyl)-5-(tetrahydro-2H- pyran-4-yl)-1H-indol-1-yl)-2- methylcyclopropyl)-1,2,4- oxadiazol-5(4H)-one | 875.5 |
| 69 | 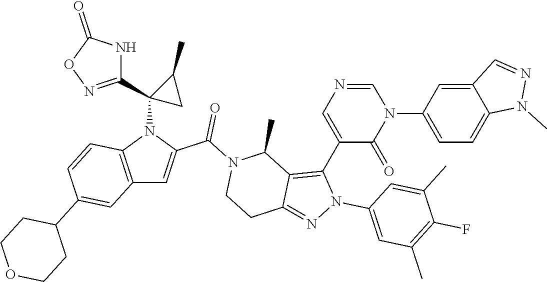 [SMILES: Cc1cc(-n2nc3c(c2-c2cncn(-c4ccc5c(cnn5C)c4)c2=O)[C@H](C)N(C(=O)c2cc4cc(C5CCOCC5)ccc4n2[C@@]2(c4noc(=O)[nH]4)C[C@@H]2C)CC3)cc(C)c1F] | 3-((1S,2S)-1-(2-((S)-2-(4- fluoro-3,5-dimethylphenyl)-4- methyl-3-(1-(1-methyl-1H- indazol-5-yl)-6-oxo-1,6- dihydropyrimidin-5-yl)-4,5,6,7- tetrahydro-2H-pyrazolo[4,3- c]pyridine-5-carbonyl)-5- (tetrahydro-2H-pyran-4-yl)-1H- indol-1-yl)-2- methylcyclopropyl)-1,2,4- oxadiazol-5(4H)-one | 849.6 |
| 70 | 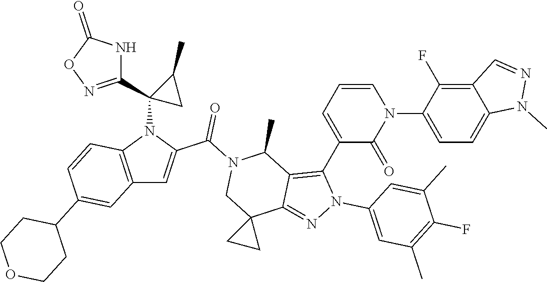 [SMILES: Cc1cc(-n2nc3c(c2-c2cccn(-c4ccc5c(cnn5C)c4F)c2=O)[C@H](C)N(C(=O)c2cc4cc(C5CCOCC5)ccc4n2[C@@]2(c4noc(=O)[nH]4)C[C@@H]2C)CC32CC2)cc(C)c1F] | 3-((1S,2S)-1-(2-((S)-3′-(1-(4- fluoro-1-methyl-1H-indazol-5- yl)-2-oxo-1,2-dihydropyridin-3- yl)-2′-(4-fluoro-3,5- dimethylphenyl)-4′-methyl- 2′,4′,5′,6′- tetrahydrospiro[cyclopropane- 1,7′-pyrazolo[4,3-c]pyridine]- 5′-carbonyl)-5-(tetrahydro-2H- pyran-4-yl)-1H-indol-1-yl)-2- methylcyclopropyl)-1,2,4- oxadiazol-5(4H)-one | 892.6 |
| 71 | 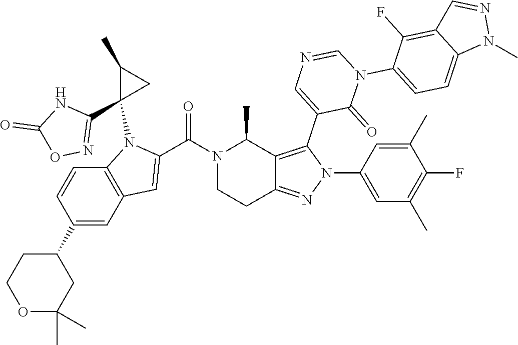 [SMILES: Cc1cc(-n2nc3c(c2-c2cncn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | 3-((1S,2S)-1-(5-((S)-2,2- dimethyltetrahydro-2H-pyran- 4-yl)-2-((S)-3-(1-(4-fluoro-1- methyl-1H-indazol-5-yl)-6-oxo- 1,6-dihydropyrimidin-5-yl)-2- (4-fluoro-3,5-dimethylphenyl)- 4-methyl-4,5,6,7-tetrahydro- 2H-pyrazolo[4,3-c]pyridine-5- carbonyl)-1H-indol-1-yl)-2- methylcyclopropyl)-1,2,4- oxadiazol-5(4H)-one | 895.6 |
| 332 | 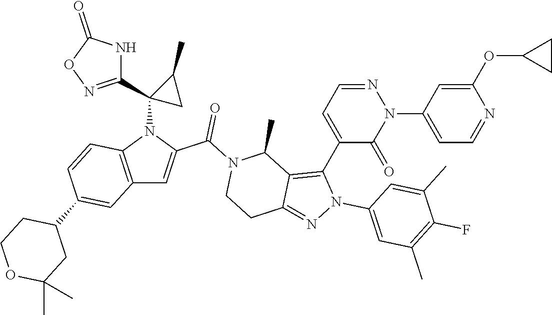 | 3-((1S,2S)-1-(2-((4S)-3-(2-(2- cyclopropoxypyridin-4-yl)-3- oxo-2,3-dihydropyridazin-4-yl)- 2-(4-fluoro-3,5- dimethylphenyl)-4-methyl- 4,5,6,7-tetrahydro-2H- pyrazolo[4,3-c]pyridine-5- carbonyl)-5-((S)-2,2- dimethyltetrahydro-2H-pyran- 4-yl)-1H-indol-1-yl)-2- methylcyclopropyl)-1,2,4- oxadiazol-5(4H)-one | 880.4 |
| 340 | 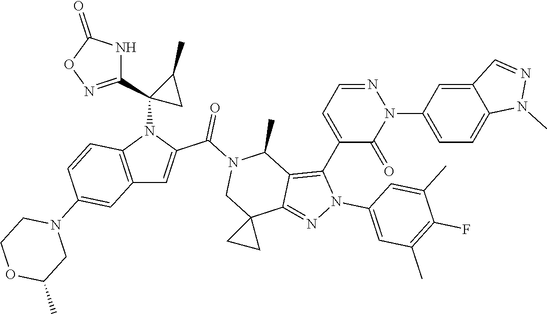 [SMILES: Cc1cc(-n2nc3c(c2-c2ccnn(-c4ccc5c(cnn5C)c4)c2=O)[C@H](C)N(C(=O)c2cc4cc(N5CCO[C@@H](C)C5)ccc4n2[C@@]2(c4noc(=O)[nH]4)C[C@@H]2C)CC32CC2)cc(C)c1F] | 1-((S)-1-(2,2-difluoroethyl)-5′- (5-((S)-2,2-dimethyltetrahydro- 2H-pyran-4-yl)-1-((1S,2S)-2- methyl-1-(5-oxo-4,5-dihydro- 1,2,4-oxadiazol-3- yl)cyclopropyl)-1H-indole-2- carbonyl)-2′-(4-fluoro-3,5- dimethylphenyl)-4′-methyl- 2′,4′,5′,6′- tetrahydrospiro[azetidine-3,7′- pyrazolo[4,3-c]pyridin]-3′-yl)- N-(4-fluoro-1-methyl-1H- indazol-5-yl)cyclopropane-1- carboxamide | 890.6 |
| 341 | 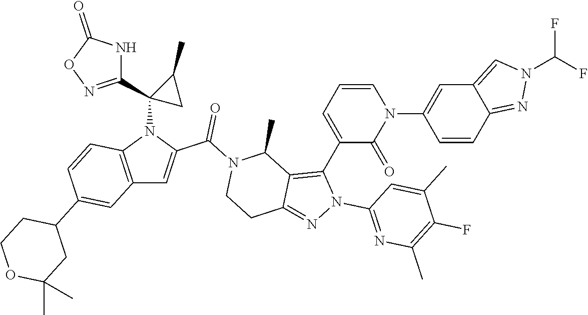 [SMILES: Cc1cc(-n2nc3c(c2-c2cccn(-c4ccc5nn(C(F)F)cc5c4)c2=O)[C@H](C)N(C(=O)c2cc4cc(C5CCOC(C)(C)C5)ccc4n2[C@@]2(c4noc(=O)[nH]4)C[C@@H]2C)CC3)nc(C)c1F] | 3-((1S,2S)-1-(2-((4S)-3-(1-(2- (difluoromethyl)-2H-indazol-5- yl)-2-oxo-1,2-dihydropyridin-3- yl)-2-(5-fluoro-4,6- dimethylpyridin-2-yl)-4- methyl-4,5,6,7-tetrahydro-2H- pyrazolo[4,3-c]pyridine-5- carbonyl)-5-(2,2- dimethyltetrahydro-2H-pyran- 4-yl)-1H-indol-1-yl)-2- methylcyclopropyl)-1,2,4- oxadiazol-5(4H)-one | 913.6 |
| 342 | 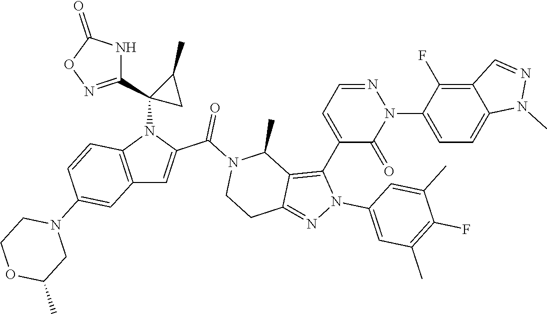 [SMILES: Cc1cc(-n2nc3c(c2-c2ccnn(-c4ccc5c(cnn5C)c4F)c2=O)[C@H](C)N(C(=O)c2cc4cc(N5CCO[C@@H](C)C5)ccc4n2[C@@]2(c4noc(=O)[nH]4)C[C@@H]2C)CC3)cc(C)c1F] | 3-((1S,2S)-1-(2-((4S)-3-(2-(4- fluoro-1-methyl-1H-indazol-5- yl)-3-oxo-2,3-dihydropyridazin- 4-yl)-2-(4-fluoro-3,5- dimethylphenyl)-4-methyl- 4,5,6,7-tetrahydro-2H- pyrazolo[4,3-c]pyridine-5- carbonyl)-5-((S)-2- methylmorpholino)-1H-indol-1- yl)-2-methylcyclopropyl)-1,2,4- oxadiazol-5(4H)-one | 882.6 |
| 343 | 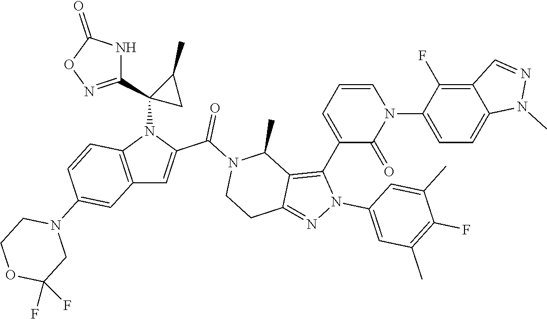 [SMILES: Cc1cc(-n2nc3c(c2-c2cccn(-c4ccc5c(cnn5C)c4F)c2=O)[C@H](C)N(C(=O)c2cc4cc(N5CCOC(F)(F)C5)ccc4n2[C@@]2(c4noc(=O)[nH]4)C[C@@H]2C)CC3)cc(C)c1F] | 3-((1S,2S)-1-(5-(2,2- difluoromorpholino)-2-((4S)-3- (1-(4-fluoro-1-methyl-1H- indazol-5-yl)-2-oxo-1,2- dihydropyridin-3-yl)-2-(4- fluoro-3,5-dimethylphenyl)-4- methyl-4,5,6,7-tetrahydro-2H- pyrazolo[4,3-c]pyridine-5- carbonyl)-1H-indol-1-yl)-2- methylcyclopropyl)-1,2,4- oxadiazol-5(4H)-one | 903.6 |
| 344 | 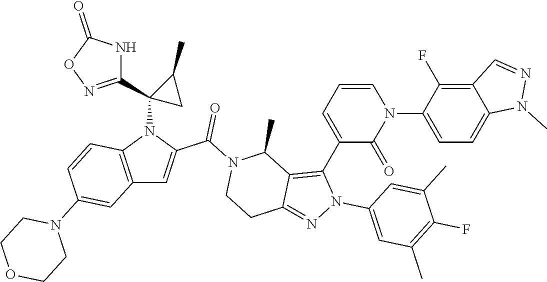 [SMILES: Cc1cc(-n2nc3c(c2-c2cccn(-c4ccc5c(cnn5C)c4F)c2=O)[C@H](C)N(C(=O)c2cc4cc(N5CCOCC5)ccc4n2[C@@]2(c4noc(=O)[nH]4)C[C@@H]2C)CC3)cc(C)c1F] | 3-((1S,2S)-1-(2-((4S)-3-(1-(4- fluoro-1-methyl-1H-indazol-5- yl)-2-oxo-1,2-dihydropyridin-3- yl)-2-(4-fluoro-3,5- dimethylphenyl)-4-methyl- 4,5,6,7-tetrahydro-2H- pyrazolo[4,3-c]pyridine-5- carbonyl)-5-morpholino-1H- indol-1-yl)-2- methylcyclopropyl)-1,2,4- oxadiazol-5(4H)-one | 867.8 |
| 345 | 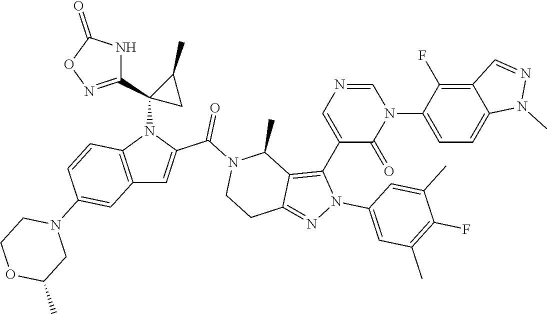 [SMILES: Cc1cc(-n2nc3c(c2-c2cncn(-c4ccc5c(cnn5C)c4F)c2=O)[C@H](C)N(C(=O)c2cc4cc(N5CCO[C@@H](C)C5)ccc4n2[C@@]2(c4noc(=O)[nH]4)C[C@@H]2C)CC3)cc(C)c1F] | 3-((1S,2S)-1-(2-((4S)-3-(1-(4- fluoro-1-methyl-1H-indazol-5- yl)-6-oxo-1,6- dihydropyrimidin-5-yl)-2-(4- fluoro-3,5-dimethylphenyl)-4- methyl-4,5,6,7-tetrahydro-2H- pyrazolo[4,3-c]pyridine-5- carbonyl)-5-((S)-2- methylmorpholino)-1H-indol-1- yl)-2-methylcyclopropyl)-1,2,4- oxadiazol-5(4H)-one | 882.6 |
| 346 | 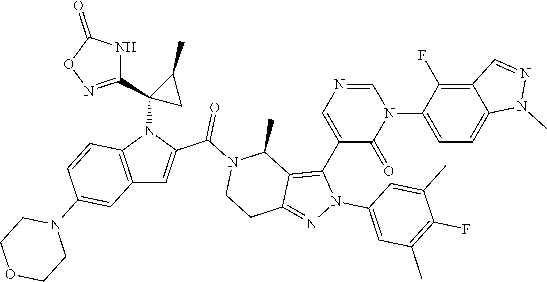 [SMILES: Cc1cc(-n2nc3c(c2-c2cncn(-c4ccc5c(cnn5C)c4F)c2=O)[C@H](C)N(C(=O)c2cc4cc(N5CCOCC5)ccc4n2[C@@]2(c4noc(=O)[nH]4)C[C@@H]2C)CC3)cc(C)c1F] | 3-((1S,2S)-1-(2-((4S)-3-(1-(4- fluoro-1-methyl-1H-indazol-5- yl)-6-oxo-1,6- dihydropyrimidin-5-yl)-2-(4- fluoro-3,5-dimethylphenyl)-4- methyl-4,5,6,7-tetrahydro-2H- pyrazolo[4,3-c]pyridine-5- carbonyl)-5-morpholino-1H- indol-1-yl)-2- methylcyclopropyl)-1,2,4- oxadiazol-5(4H)-one | 868.8 |
| 347 | 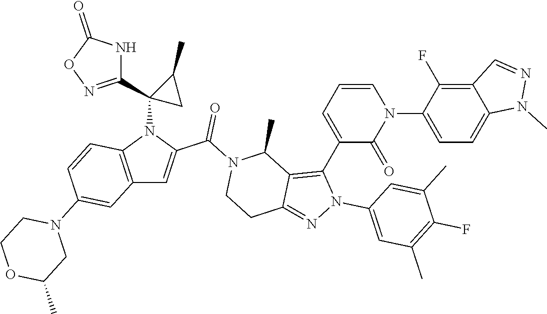 [SMILES: Cc1cc(-n2nc3c(c2-c2cccn(-c4ccc5c(cnn5C)c4F)c2=O)[C@H](C)N(C(=O)c2cc4cc(N5CCO[C@@H](C)C5)ccc4n2[C@@]2(c4noc(=O)[nH]4)C[C@@H]2C)CC3)cc(C)c1F] | 3-((1S,2S)-1-(2-((4S)-3-(1-(4- fluoro-1-methyl-1H-indazol-5- yl)-2-oxo-1,2-dihydropyridin-3- yl)-2-(4-fluoro-3,5- dimethylphenyl)-4-methyl- 4,5,6,7-tetrahydro-2H- pyrazolo[4,3-c]pyridine-5- carbonyl)-5-((S)-2- methylmorpholino)-1H-indol-1- yl)-2-methylcyclopropyl)-1,2,4- oxadiazol-5(4H)-one | 881.6 |
| 348 | 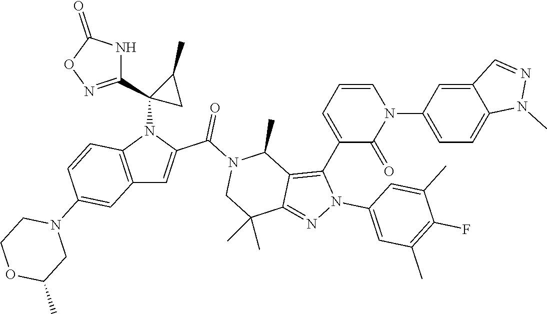 [SMILES: Cc1cc(-n2nc3c(c2-c2cccn(-c4ccc5c(cnn5C)c4)c2=O)[C@H](C)N(C(=O)c2cc4cc(N5CCO[C@@H](C)C5)ccc4n2[C@@]2(c4noc(=O)[nH]4)C[C@@H]2C)CC3(C)C)cc(C)c1F] | 3-((1S,2S)-1-(2-((4S)-2-(4- fluoro-3,5-dimethylphenyl)- 4,7,7-trimethyl-3-(1-(1-methyl- 1H-indazol-5-yl)-2-oxo-1,2- dihydropyridin-3-yl)-4,5,6,7- tetrahydro-2H-pyrazolo[4,3- c]pyridine-5-carbonyl)-5-((S)- 2-methylmorpholino)-1H-indol- 1-yl)-2-methylcyclopropyl)- 1,2,4-oxadiazol-5(4H)-one | 891.6 |
| 349 | 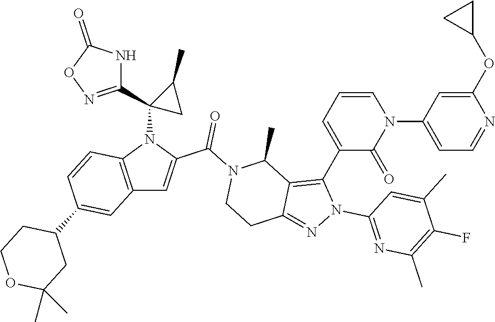 | 3-((1S,2S)-1-(2-((4S)-3-(2′- cyclopropoxy-2-oxo-2H-[1,4′- bipyridin]-3-yl)-2-(5-fluoro- 4,6-dimethylpyridin-2-yl)-4- methyl-4,5,6,7-tetrahydro-2H- pyrazolo[4,3-c]pyridine-5- carbonyl)-5-((S)-2,2- dimethyltetrahydro-2H-pyran- 4-yl)-1H-indol-1-yl)-2- methylcyclopropyl)-1,2,4- oxadiazol-5(4H)-one | 880.6 |

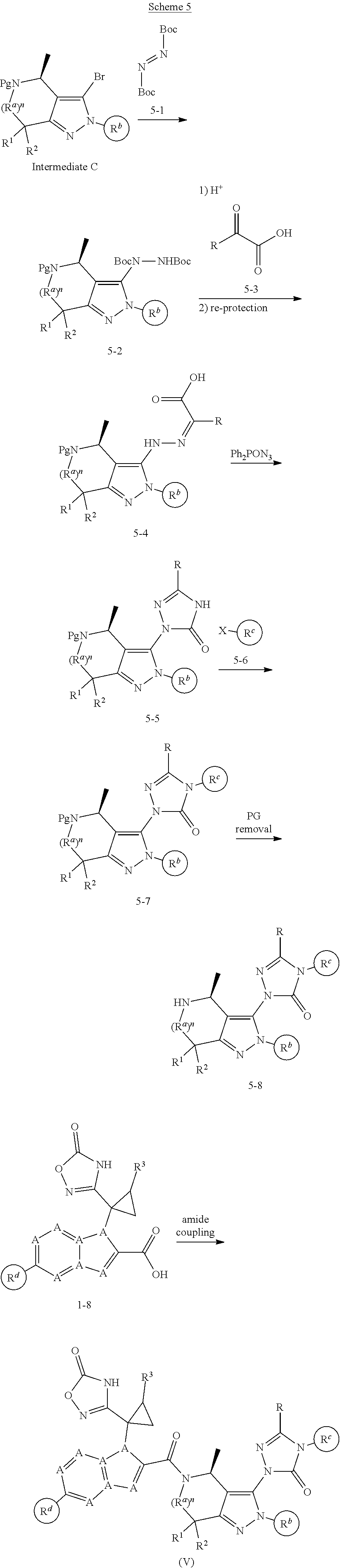

Compounds of formula (V) are prepared from intermediate C by coupling with di-tert-butyl azodicarboxylate (5-1) through the generation of an organometallic intermediate (e.g., addition of nBuLi) to provide 5-2. Subsequently, the Boc groups of 5-2 are removed (e.g., acidic conditions) and the resulting hydrazine is condensed with 2-oxoacetic acid or 2-oxoalkanoic acids (5-3). The alkyl nitrogen is re-protected (e.g., Boc₂O under basic conditions) to provide imine 5-4. Treatment of 5-4 with diphenylphosphoryl azide (or related azide reagent) provides 1,2,4-triazol-3-one 5-5. Cross-coupling on 5-5 with halides, boronic acids, boronates, or sulfonates (5-6) via transition metal catalyzed cross-coupling (e.g., Pd—or Cu-catalysis) or alkylation (e.g., basic) conditions results in 5-7. Removal of the protecting group (e.g., acidic conditions) of 5-7 provides amine 5-8 that is then coupled with carboxylic acid 1-8 to provide compounds of formula (V).

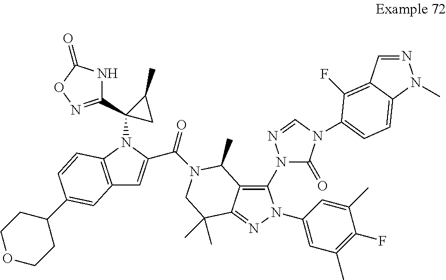 [SMILES: Cc1cc(-n2nc3c(c2-n2ncn(-c4ccc5c(cnn5C)c4F)c2=O)[C@H](C)N(C(=O)c2cc4cc(C5CCOCC5)ccc4n2[C@@]2(c4noc(=O)[nH]4)C[C@@H]2C)CC3(C)C)cc(C)c1F]

### 3-((1S,2S)-1-(2-((S)-3-(4-(4-fluoro-1-methyl-1H-indazol-5-yl)-5-oxo-4,5-dihydro-1H-1,2,4-triazol-1-yl)-2-(4-fluoro-3,5-dimethylphenyl)-4,7,7-trimethyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one (Scheme 5)

### Step 1: di-tert-butyl (S)-1-(5-(tert-butoxycarbonyl)-2-(4-fluoro-3,5-dimethylphenyl-4,7,7-trimethyl-4,5,6,7-tetrahydro-2H-pyrazolo[4,3-c]pyridin-3-yl)hydrazine-1,2-dicarboxylate

To a solution of tert-butyl (S)-3-bromo-2-(4-fluoro-3,5-dimethylphenyl)-4,7,7-trimethyl-2,4,6,7-tetrahydro-5H-pyrazolo[4,3-c]pyridine-5-carboxylate (800 mg, 1.72 mmol) in THF (8.0 mL) was added n-BuLi (2.5M in hexane) (0.823 mL, 2.06 mmol) at −78° C. under an atmosphere of nitrogen. The mixture was stirred at −78° C. for 0.5 h. Then, the mixture was added a solution of di-tert-butyl (E)-diazene-1,2-dicarboxylate (790 mg, 3.43 mmol) in THF (4 mL) at −78° C., and stirred for 1 h. The mixture was quenched by the addition of sat. aq. NH₄Cl solution (15 mL) at 0° C. and extracted with EtOAc (3×30 mL). The combined organic layers were washed with brine (10 mL), dried over anhydrous Na₂SO₄, filtered, and concentrated under reduced pressure. The crude residue was purified by SiO₂ chromatography (Pet.ether:EtOAc=5:1) to provide the title compound. ¹H NMR (400 MHz, chloroform-d) δ 7.19-6.87 (m, 2H), 6.40-5.72 (m, 1H), 5.62-5.34 (m, 1H), 4.07-3.73 (m, 1H), 3.05-2.82 (m, 1H), 2.28 (br s, 6H), 1.51 (br d, J=11.2 Hz, 27H), 1.43 (br s, 3H), 1.40-1.33 (m, 6H). MS (ESI) m/z: 618.2 [M+H]+.

### Step 2: (S,Z)-2-(2-(5-(tert-butoxycarbonyl)-2-(4-fluoro-3,5-dimethylphenyl)-4,7,7-trimethyl-4,5,6,7-tetrahydro-2H-pyrazolo[4,3-c]pyridin-3-yl)hydrazineylidene)acetic acid and (S,E)-2-(2-(5-(tert-butoxycarbonyl)-2-(4-fluoro-3,5-dimethylphenyl)-4,7,7-trimethyl-4,5,6,7-tetrahydro-2H-pyrazolo[4,3-c]pyridin-3-yl)hydrazineylidene)acetic acid

To a solution of di-tert-butyl (S)-1-(5-(tert-butoxycarbonyl)-2-(4-fluoro-3,5-dimethylphenyl)-4,7,7-trimethyl-4,5,6,7-tetrahydro-2H-pyrazolo[4,3-c]pyridin-3-yl)hydrazine-1,2-dicarboxylate (340 mg, 0.55 mmol) in HCl (5.0 mL) was added glyoxylic acid monohydrate (51 mg, 0.55 mmol), and the reaction mixture was stirred for 16 h at 15° C. Then, the mixture was concentrated by lyophilization to provide the title compound. LCMS (ESI) m/z: 374.1 [M+1]+. To a solution of (S,Z)-2-(2-(2-(4-fluoro-3,5-dimethylphenyl)-4,7,7-trimethyl-4,5,6,7-tetrahydro-2H-pyrazolo[4,3-c]pyridin-3-yl)hydrazineylidene)acetic acid and (S,E)-2-(2-(2-(4-fluoro-3,5-dimethylphenyl)-4,7,7-trimethyl-4,5,6,7-tetrahydro-2H-pyrazolo[4,3-c]pyridin-3-yl)hydrazineylidene)acetic acid (320 mg, 0.857 mmol) in THF (2 mL) and water (2 mL) was added Na₂CO₃ (136 mg, 1.29 mmol) and Boc₂O (0.199 mL, 0.857 mmol), and the mixture was stirred at 25° C. for 16 h. Then, the reaction mixture was diluted with EtOAc (10 mL) and aqueous 1 M HCl (5 mL). The organic layer was separated, and the aqueous phase was extracted with EtOAc (3×10 mL). The organic layers were combined, washed with brine, dried over Na₂SO₄, filtered, and concentrated under reduced pressure to afford the title compound. MS (ESI) m/z 474.5 [M+1]+.

### Step 3: tert-butyl (S)-2-(4-fluoro-3,5-dimethylphenyl)-4,7,7-trimethyl-3-(5-oxo-4,5-dihydro-1H-1,2,4-triazol-1-yl)-2,4,6,7-tetrahydro-5H-pyrazolo[4,3-c]pyridine-5-carboxylate

A mixture of (S,Z)-2-(2-(5-(tert-butoxycarbonyl)-2-(4-fluoro-3,5-dimethylphenyl)-4,7,7-trimethyl-4,5,6,7-tetrahydro-2H-pyrazolo[4,3-c]pyridin-3-yl)hydrazineylidene)acetic acid and (S,E)-2-(2-(5-(tert-butoxycarbonyl)-2-(4-fluoro-3,5-dimethylphenyl)-4,7,7-trimethyl-4,5,6,7-tetrahydro-2H-pyrazolo[4,3-c]pyridin-3-yl)hydrazineylidene)acetic acid (300 mg, 0.634 mmol), diphenylphosphoryl azide (192 mg, 0.697 mmol), and TEA (192 mg, 1.90 mmol) in toluene (4.0 mL) was stirred at 100° C. via microwave irradiation for 20 min. Then, the reaction mixture was concentrated under reduced pressure, and the crude residue was purified by reverse-phase HPLC (58% to 78% MeCN/water+0.1% TFA). MS (ESI) m/z: 471.2 [M+H]+.

### Step 4: tert-butyl (S)-3-(4-(4-fluoro-1-methyl-1H-indazol-5-yl)-5-oxo-4,5-dihydro-1H-1,2,4-triazol-1-yl)-2-(4-fluoro-3,5-dimethylphenyl)-4,7,7-trimethyl-2,4,6,7-tetrahydro-5H-pyrazolo[4,3-c]pyridine-5-carboxylate

To a solution of tert-butyl (S)-2-(4-fluoro-3,5-dimethylphenyl)-4,7,7-trimethyl-3-(5-oxo-4,5-dihydro-1H-1,2,4-triazol-1-yl)-2,4,6,7-tetrahydro-5H-pyrazolo[4,3-c]pyridine-5-carboxylate (60 mg, 0.13 mmol), (4-fluoro-1-methyl-1H-indazol-5-yl)boronic acid (50 mg, 0.26 mmol), 4A MS (10 mg), and TEA (0.071 mL, 0.51 mmol) was added copper(II) acetate monohydrate (5.1 mg, 0.026 mmol) in DCE (1.0 mL). The mixture was stirred at 40° C. for 16 h under an atmosphere of oxygen. Then, the reaction mixture was purified by prep-TLC (SiO₂; 33% EtOAc/Pet.ether) to provide the title compound. MS (ESI) m/z: 619.4 [M+H]+.

### Step 5: (S)-4-(4-fluoro-1-methyl-1H-indazol-5-yl)-2-(2-(4-fluoro-3,5-dimethylphenyl)-4,7,7-trimethyl-4,5,6,7-tetrahydro-2H-pyrazolo[4,3-c]pyridin-3-yl)-2,4-dihydro-3H-1,2,4-triazol-3-one

A solution of tert-butyl (S)-3-(4-(4-fluoro-1-methyl-1H-indazol-5-yl)-5-oxo-4,5-dihydro-1H-1,2,4-triazol-1-yl)-2-(4-fluoro-3,5-dimethylphenyl)-4,7,7-trimethyl-2,4,6,7-tetrahydro-5H-pyrazolo[4,3-c]pyridine-5-carboxylate (27 mg, 0.044 mmol) in 2 M HCl/dioxane (1 mL) was stirred for 2 h at 20° C. Then, the reaction mixture was filtered, and concentrated under reduced pressure to provide the title compound. MS (ESI) m/z: 519.3 [M+H]+.

### Step 6: 3-((1S,2S)-1-(2-((S)-3-(4-(4-fluoro-1-methyl-1H-indazol-5-yl)-5-oxo-4,5-dihydro-1H-1,2,4-triazol-1-yl)-2-(4-fluoro-3,5-dimethylphenyl)-4,7,7-trimethyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one

To a solution of 1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carboxylic acid (16 mg, 0.042 mmol) and (S)-4-(4-fluoro-1-methyl-1H-indazol-5-yl)-2-(2-(4-fluoro-3,5-dimethylphenyl)-4,7,7-trimethyl-4,5,6,7-tetrahydro-2H-pyrazolo[4,3-c]pyridin-3-yl)-2,4-dihydro-3H-1,2,4-triazol-3-one (22 mg, 0.042 mmol) in DMF (0.10 mL) was added DIPEA (0.022 mL, 0.13 mmol). The mixture was stirred at 20° C. for 10 min. Then, HATU (24 mg, 0.064 mmol) was added, and the mixture was stirred at 20° C. for 16 h. Then, the crude mixture was purified by reverse-phase HPLC (purification 1: 62% to 82% MeCN/water+0.1% TFA then purification 2: 33% to 63% MeCN/water+0.05% NH₃H₂O+10 mM NH₄HCO₃) to provide the title compound. ¹H NMR (400 MHz, methanol-d4) δ=8.36-8.09 (m, 2H), 7.57 (br s, 3H), 7.43 (br d, J=9.3 Hz, 2H), 7.18-7.05 (m, 2H), 7.01-6.81 (m, 1H), 5.97-5.61 (m, 1H), 4.17-3.95 (m, 6H), 3.67-3.41 (m, 3H), 2.96-2.79 (m, 1H), 2.32-2.24 (m, 6H), 1.86-1.75 (m, 6H), 1.68-1.53 (m, 5H), 1.47 (br d, J=18.4 Hz, 4H), 1.37-1.28 (m, 1H), 1.22 (br d, J=5.4 Hz, 2H), 1.14-1.06 (m, 1H). MS (ESI) m/z: 884.3 [M+H]+. The following examples in table 5 were prepared according to Scheme 5 using the procedures outlined in the synthesis of example 72.

TABLE 5

| | | | MS |
|---|---|---|---|
| Ex. | Structure | Name | (M + 1) |
| 73 | 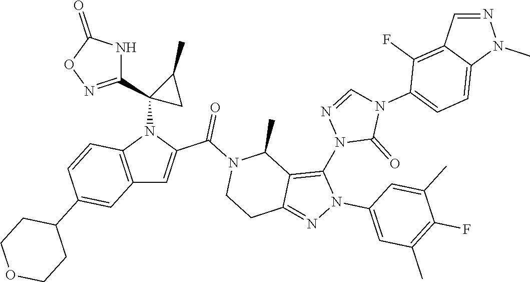 [SMILES: Cc1cc(-n2nc3c(c2-n2ncn(-c4ccc5c(cnn5C)c4F)c2=O)[C@H](C)N(C(=O)c2cc4cc(C5CCOCC5)ccc4n2[C@@]2(c4noc(=O)[nH]4)C[C@@H]2C)CC3)cc(C)c1F] | 3-((1S,2S)-1-(2-((S)-3-(4-(4- fluoro-1-methyl-1H-indazol-5- yl)-5-oxo-4,5-dihydro-1H- 1,2,4-triazol-1-yl)-2-(4-fluoro- 3,5-dimethylphenyl)-4-methyl- 4,5,6,7-tetrahydro-2H- pyrazolo[4,3-c]pyridine-5- carbonyl)-5-(tetrahydro-2H- pyran-4-yl)-1H-indol-1-yl)-2- methylcyclopropyl)-1,2,4- oxadiazol-5(4H)-one | 856.2 |
| 74 | 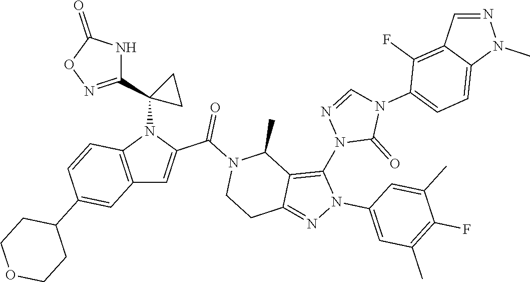 | (S)-3-(1-(2-(3-(4-(4-fluoro-1- methyl-1H-indazol-5-yl)-5-oxo- 4,5-dihydro-1H-1,2,4-triazol-1- yl)-2-(4-fluoro-3,5- dimethylphenyl)-4-methyl- 4,5,6,7-tetrahydro-2H- pyrazolo[4,3-c]pyridine-5- carbonyl)-5-(tetrahydro-2H- pyran-4-yl)-1H-indol-1- yl)cyclopropyl)-1,2,4- oxadiazol-5(4H)-one | 842.2 |
| 75 | 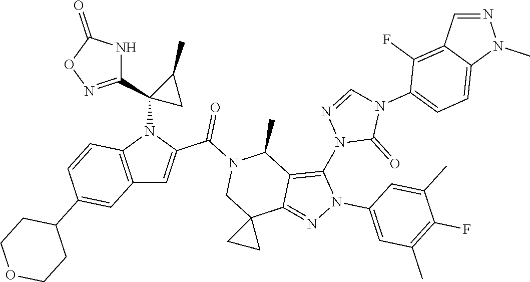 [SMILES: Cc1cc(-n2nc3c(c2-n2ncn(-c4ccc5c(cnn5C)c4F)c2=O)[C@H](C)N(C(=O)c2cc4cc(C5CCOCC5)ccc4n2[C@@]2(c4noc(=O)[nH]4)C[C@@H]2C)CC32CC2)cc(C)c1F] | 3-((1S,2S)-1-(2-((S)-3′-(4-(4- fluoro-1-methyl-1H-indazol-5- yl)-5-oxo-4,5-dihydro-1H- 1,2,4-triazol-1-yl)-2′-(4-fluoro- 3,5-dimethylphenyl)-4′-methyl- 2′,4′,5′,6′- tetrahydrospiro[cyclopropane- 1,7′-pyrazolo[4,3-c]pyridine]- 5′-carbonyl)-5-(tetrahydro-2H- pyran-4-yl)-1H-indol-1-yl)-2- methylcyclopropyl)-1,2,4- oxadiazol-5(4H)-one | 882.2 |
| 76 | 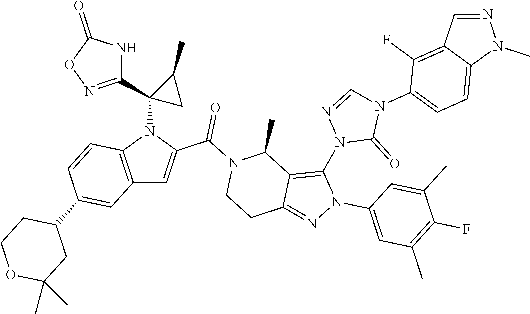 | 3-((1S,2S)-1-(5-((S)-2,2- dimethyltetrahydro-2H-pyran- 4-yl)-2-((S)-3-(4-(4-fluoro-1- methyl-1H-indazol-5-yl)-5-oxo- 4,5-dihydro-1H-1,2,4-triazol-1- yl)-2-(4-fluoro-3,5- dimethylphenyl)-4-methyl- 4,5,6,7-tetrahydro-2H- pyrazolo[4,3-c]pyridine-5- carbonyl)-1H-indol-1-yl)-2- methylcyclopropyl)-1,2,4- oxadiazol-5(4H)-one | 884.2 |
| 77 | 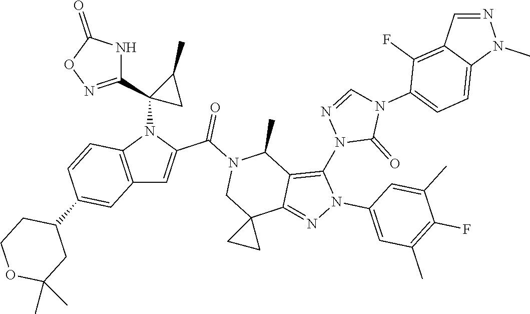 | 3-((1S,2S)-1-(5-((S)-2,2- dimethyltetrahydro-2H-pyran- 4-yl)-2-((S)-3′-(4-(4-fluoro-1- methyl-1H-indazol-5-yl)-5-oxo- 4,5-dihydro-1H-1,2,4-triazol-1- yl)-2′-(4-fluoro-3,5- dimethylphenyl)-4′-methyl- 2′,4′,5′,6′- tetrahydrospiro[cyclopropane- 1,7′-pyrazolo[4,3-c]pyridine]- 5′-carbonyl)-1H-indol-1-yl)-2- methylcyclopropyl)-1,2,4- oxadiazol-5(4H)-one | 910.3 |
| 325 | 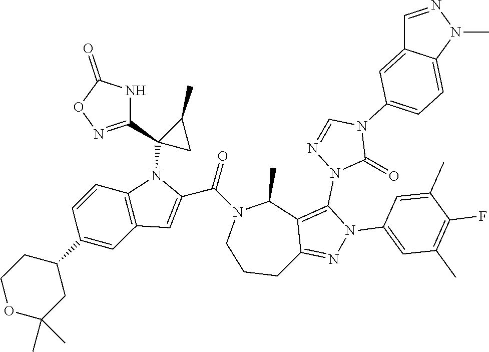 | 3-[(1S,2S)-1-[5-[(4S)-2,2- dimethyltetrahydropyran-4-yl]- 2-[(4S)-2-(4-fluoro-3,5- dimethyl-phenyl)-4-methyl-3- [4-(1-methylindazol-2-ium-5- yl)-5-oxo-1,2,4-triazol-1-yl]- 4,6,7,8-tetrahydropyrazolo[4,3- c]azepine-5-carbonyl]indol-1- yl]-2-methyl-cyclopropyl]-4H- 1,2,4-oxadiazol-5-one | 880.4 |
| 326 | 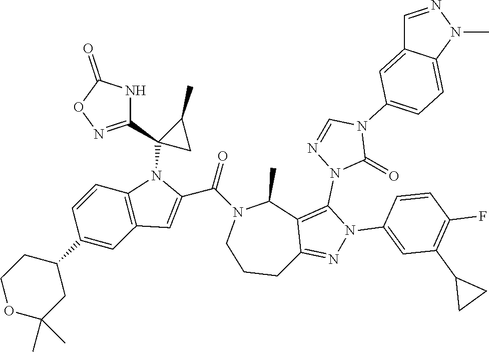 | 3-[(1S,2S)-1-[5-[(4S)-2,2- dimethyltetrahydropyran-4-yl]- 2-[(4S)-2-(4-fluoro-3,5- dimethyl-phenyl)-4-methyl-3- [4-(1-methylindazol-2-ium-5- yl)-5-oxo-1,2,4-triazol-1-yl]- 4,6,7,8-tetrahydropyrazolo[4,3- c]azepine-5-carbonyl]indol-1- yl]-2-methyl-cyclopropyl]-4H- 1,2,4-oxadiazol-5-one | 910.4 |

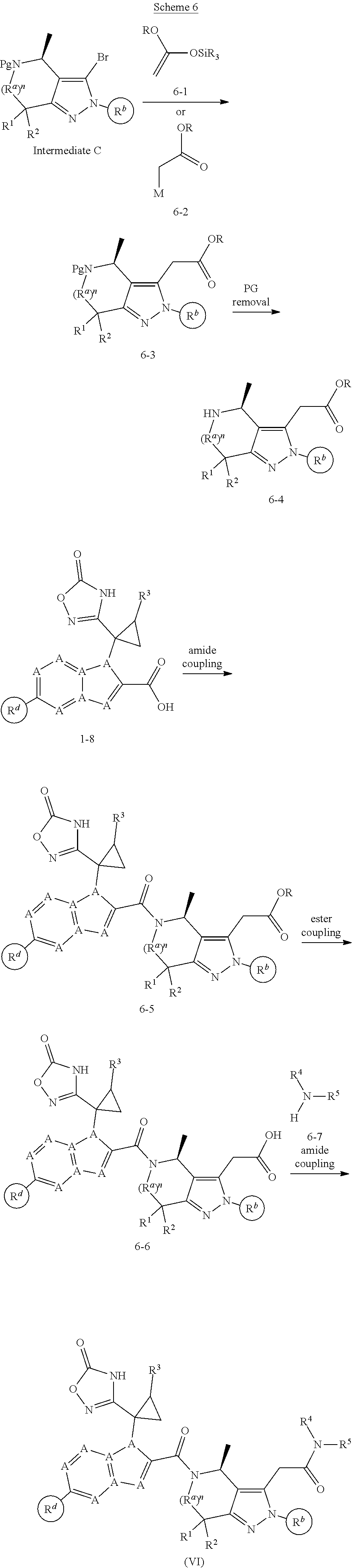

Compounds of formula (VI) are prepared from intermediate C by coupling with silyl enol ethers (6-1) or metal enolates such as zinc enolates (6-2, depicted arbitrarily as one possible tautomer) via transition metal catalyzed cross-coupling conditions (e.g., Pd-catalysis) to provide 6-3. Removal of the protecting group (e.g., acidic conditions) of 6-3 provides amine 6-4 that is then coupled with carboxylic acid 1-8 to provide intermediate 6-5. Subsequently, the ester of 6-5 is removed (e.g., basic aqueous conditions) to furnish carboxylic acid 6-6 that is then coupled with amines (6-7) to provide compounds of formula (VI).

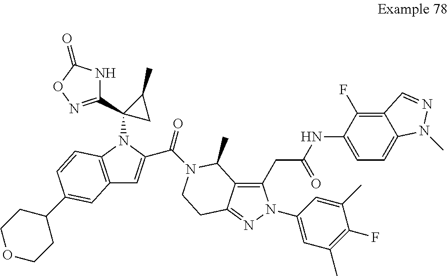 [SMILES: Cc1cc(-n2nc3c(c2CC(=O)Nc2ccc4c(cnn4C)c2F)[C@H](C)N(C(=O)c2cc4cc(C5CCOCC5)ccc4n2[C@@]2(c4noc(=O)[nH]4)C[C@@H]2C)CC3)cc(C)c1F]

### N-(4-fluoro-1-methyl-1H-indazol-5-yl)-2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)acetamide (Scheme 6)

### Step 1: tert-butyl (S)-2-(4-fluoro-3,5-dimethylphenyl)-3-(2-methoxy-2-oxoethyl)-4-methyl-2,4,6,7-tetrahydro-5H-pyrazolo[4,3-c]pyridine-5-carboxylate

Under a nitrogen atmosphere, to a mixture of tert-butyl (S)-3-bromo-2-(4-fluoro-3,5-dimethylphenyl)-4-methyl-2,4,6,7-tetrahydro-5H-pyrazolo[4,3-c]pyridine-5-carboxylate (281 mg, 0.64 mmol), bis(tri-t-butylphosphine)palladium(0) (33 mg, 0.064 mmol) and lithium fluoride (100 mg, 3.9 mmol) in DMF (3.2 mL) were added tert-butyl((1-methoxyvinyl)oxy)dimethylsilane (424 L, 1.92 mmol), and then the reaction mixture was stirred at 100° C. overnight. The reaction mixture was cooled to rt, then water and EtOAc were added. The layers were separated, and the aqueous layer was extracted with EtOAc (3×). The combined organic layers were dried with Na₂SO₄, filtered, and concentrated under reduced pressure. The crude mixture was purified by silica gel chromatography (0 to 100% EtOAc/Hex) to afford the title compound. MS (ESI) m/z 432.2 [M+H]+.

### Step 2: methyl (S)-2-(2-(4-fluoro-3,5-dimethylphenyl)-4-methyl-4,5,6,7-tetrahydro-2H-pyrazolo[4,3-c]pyridin-3-yl)acetate

Tert-butyl (S)-2-(4-fluoro-3,5-dimethylphenyl)-3-(2-methoxy-2-oxoethyl)-4-methyl-2,4,6,7-tetrahydro-5H-pyrazolo[4,3-c]pyridine-5-carboxylate (88 mg, 0.20 mmol) was dissolved in DCM (0.81 mL) and TFA (0.20 mL) at rt. The reaction was stirred at rt overnight. The reaction mixture was concentrated under reduced pressure, then EtOAc and sat. aq. Na₂CO₃ were added. The layers were separated, and the aqueous layer was extracted with EtOAc (3×). The combined organic layers were dried with Na₂SO₄, filtered, and concentrated under reduced pressure to produce the title compound. MS (ESI) m/z 332.2 [M+H]+.

### Step 3: methyl 2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)acetate

To a mixture of methyl (S)-2-(2-(4-fluoro-3,5-dimethylphenyl)-4-methyl-4,5,6,7-tetrahydro-2H-pyrazolo[4,3-c]pyridin-3-yl)acetate (725 mg, 2.19 mmol), 1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carboxylic acid (1.05 g, 2.73 mmol), and HATU (1.04 g, 2.73 mmol) in DMF (21.9 mL) was added DIPEA (1.91 mL, 10.9 mmol). The reaction mixture was stirred at room temperature overnight. Then, EtOAc and brine were added, and the layers were separated. The aqueous layer was extracted with EtOAc (3×). The combined organic layers were dried with Na₂SO₄, filtered, and concentrated under reduced pressure. The residue was purified by silica gel chromatography (0 to 100% EtOAc/hexanes) to provide the title compound. MS (ESI) m/z 697.2 [M+H]+.

### Step 4: 2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)acetic acid

To a solution of methyl 2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)acetate (1.02 g, 1.46 mmol) in THF (5.85 mL) and MeOH (2.93 mL) was added a solution of LiOH—H₂O (184 mg, 4.39 mmol) in water (5.85 mL). The mixture was stirred for 2 h then a solution of 1N aqueous HCl was added to reach pH 0. EtOAc was added, the layers separated, and the aqueous layer was extracted with EtOAc (3×). The combined organic layers were dried with Na₂SO₄, filtered, and concentrated under reduced pressure to afford the title compound. MS (ESI) m/z 683.2 [M+H]+.

### Step 5: N-(4-fluoro-1-methyl-1H-indazol-5-yl)-2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)acetamide

To a mixture of 2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)acetic acid (140 mg, 0.20 mmol), 4-fluoro-1-methyl-1H-indazol-5-amine (49 mg, 0.30 mmol), and HATU (110 mg, 0.30 mmol) in DMF (2.0 mL) was added DIPEA (0.17 mL, 0.99 mmol) at rt. The reaction mixture was stirred at rt overnight. Then, the mixture was filtered and purified by prep-HPLC (60% to 85% MeCN/H2O+0.1% formic acid gradient) to provide the title compound. ¹H NMR (500 MHz, DMSO-d6) δ 11.92-11.74 (m, 1H), 10.04-9.77 (m, 1H), 8.17-8.02 (m, 1H), 7.54-7.24 (m, 6H), 7.21-6.97 (m, 1H), 6.94-6.86 (m, 1H), 5.76-5.08 (m, 1H), 4.81-4.28 (m, 1H), 4.07-3.59 (m, 8H), 3.53-3.40 (m, 3H), 3.16-2.76 (m, 3H), 2.28-2.21 (m, 6H), 1.79-1.52 (m, 9H), 1.46-1.29 (m, 2H), 1.22-0.92 (in, 3H). MS (ESI) m/z 830.4 [M+H]+. The following examples in table 6 were prepared according to Scheme 6 using the procedures outlined in the synthesis of example 78. Additionally, Examples 331 Isomer A and B are made from intermediates I-RJ-2 isomers A and B using the last step from Scheme 6.

TABLE 6

| | | | MS |
|---|---|---|---|
| | | | (M + |
| Ex. | Structure | Name | 1) |
| 79 | 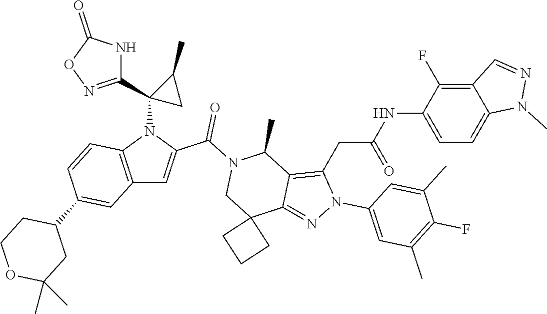 | 2-((4′S)-5′-(5-(2,2- dimethyltetrahydro-2H-pyran- 4-yl)-1-((1S,2S)-2- methyl-1-(5-oxo- 4,5-dihydro-1,2,4-oxadiazol-3- yl)cyclopropyl)-1H-indole-2- carbonyl)-2′-(4-fluoro-3,5- dimethylphenyl)-4′-methyl- 2′,4′,5′,6′- tetrahydrospiro [cyclobutane-1,7′- pyrazolo[4,3-c] pyridin]-3′-yl)-N- (4-fluoro-1-methyl- 1H-indazol-5- yl)acetamide | 898.4 |
| 80 | 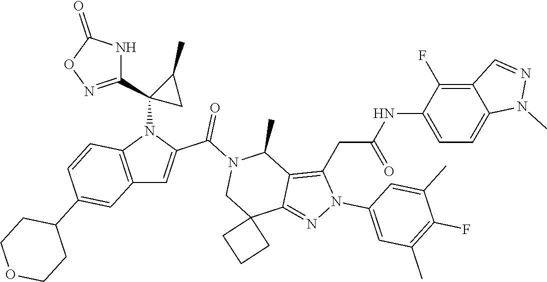 [SMILES: Cc1cc(-n2nc3c(c2CC(=O)Nc2ccc4c(cnn4C)c2F)[C@H](C)N(C(=O)c2cc4cc(C5CCOCC5)ccc4n2[C@@]2(c4noc(=O)[nH]4)C[C@@H]2C)CC32CCC2)cc(C)c1F] | N-(4-fluoro-1- methyl-1H-indazol- 5-yl)-2-((S)-2′-(4-fluoro-3,5- dimethylphenyl)- 4′-methyl-5′-(1- ((1S,2S)-2-methyl- 1-(5-oxo-4,5- dihydro-1,2,4-oxadiazol-3- yl)cyclopropyl)- 5-(tetrahydro-2H- pyran-4-yl)-1H-indole-2- carbonyl)-2′,4′,5′,6′- tetrahydrospiro [cyclobutane-1,7′- pyrazolo[4,3-c]pyridin]-3′- yl)acetamide | 870.4 |
| 81 | 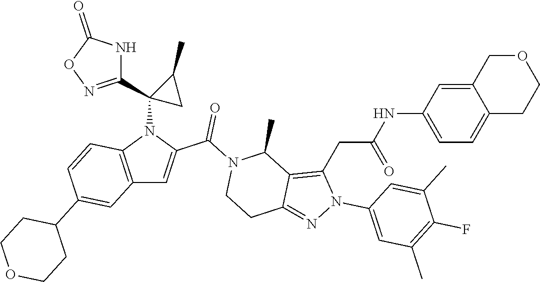 [SMILES: Cc1cc(-n2nc3c(c2CC(=O)Nc2ccc4c(c2)COCC4)[C@H](C)N(C(=O)c2cc4cc(C5CCOCC5)ccc4n2[C@@]2(c4noc(=O)[nH]4)C[C@@H]2C)CC3)cc(C)c1F] | 2-((S)-2-(4-fluoro-3,5- dimethylphenyl)- 4-methyl-5-(1- ((1S,2S)-2-methyl- 1-(5-oxo-4,5- dihydro-1,2,4-oxadiazol-3- yl)cyclopropyl)-5- (tetrahydro-2H- pyran-4-yl)-1H-indole-2- carbonyl)-4,5,6,7- tetrahydro-2H- pyrazolo[4,3-c] pyridin-3-yl)-N- (isochroman-7-yl) acetamide | 814.4 |
| 82 | 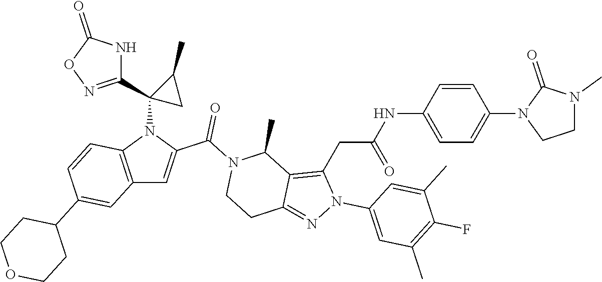 [SMILES: Cc1cc(-n2nc3c(c2CC(=O)Nc2ccc(N4CCN(C)C4=O)cc2)[C@H](C)N(C(=O)c2cc4cc(C5CCOCC5)ccc4n2[C@@]2(c4noc(=O)[nH]4)C[C@@H]2C)CC3)cc(C)c1F] | 2-((S)-2-(4-fluoro-3,5- dimethylphenyl)- 4-methyl-5-(1- ((1S,2S)-2-methyl- 1-(5-oxo-4,5- dihydro-1,2,4-oxadiazol-3- yl)cyclopropyl)- 5-(tetrahydro-2H- pyran-4-yl)-1H-indole-2- carbonyl)-4,5,6,7- tetrahydro-2H- pyrazolo[4,3-c] pyridin-3-yl)-N-(4- (3-methyl-2- oxoimidazolidin-1- yl)phenyl)acetamide | 856.4 |
| 83 | 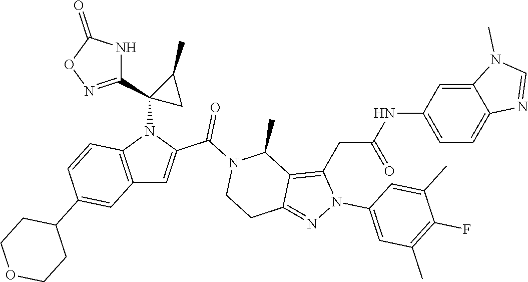 [SMILES: Cc1cc(-n2nc3c(c2CC(=O)Nc2ccc4ncn(C)c4c2)[C@H](C)N(C(=O)c2cc4cc(C5CCOCC5)ccc4n2[C@@]2(c4noc(=O)[nH]4)C[C@@H]2C)CC3)cc(C)c1F] | 2-((S)-2-(4-fluoro-3,5- dimethylphenyl)- 4-methyl-5-(1- ((1S,2S)-2-methyl- 1-(5-oxo-4,5- dihydro-1,2,4-oxadiazol-3- yl)cyclopropyl)- 5-(tetrahydro-2H- pyran-4-yl)-1H-indole-2- carbonyl)-4,5,6,7- tetrahydro-2H- pyrazolo[4,3-c] pyridin-3-yl)-N-(1- methyl-1H-benzo [d]imidazol-6- yl)acetamide | 812.4 |
| 84 | 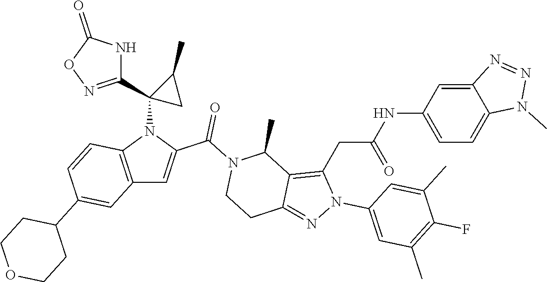 [SMILES: Cc1cc(-n2nc3c(c2CC(=O)Nc2ccc4c(c2)nnn4C)[C@H](C)N(C(=O)c2cc4cc(C5CCOCC5)ccc4n2[C@@]2(c4noc(=O)[nH]4)C[C@@H]2C)CC3)cc(C)c1F] | 2-((S)-2-(4-fluoro-3,5- dimethylphenyl)- 4-methyl-5-(1- ((1S,2S)-2-methyl- 1-(5-oxo-4,5- dihydro-1,2,4-oxadiazol-3- yl)cyclopropyl)- 5-(tetrahydro-2H- pyran-4-yl)-1H-indole-2- carbonyl)-4,5,6,7- tetrahydro-2H- pyrazolo[4,3-c] pyridin-3-yl)-N-(1- methyl-1H-benzo [d][1,2,3]triazol- 5-yl)acetamide | 813.4 |
| 85 | 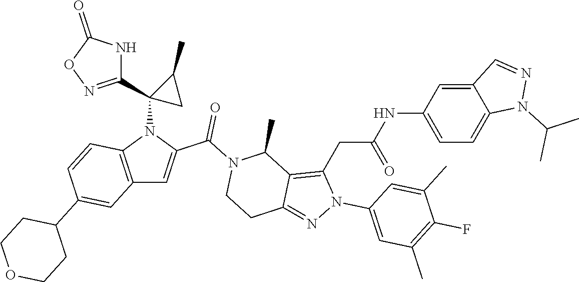 [SMILES: Cc1cc(-n2nc3c(c2CC(=O)Nc2ccc4c(cnn4C(C)C)c2)[C@H](C)N(C(=O)c2cc4cc(C5CCOCC5)ccc4n2[C@@]2(c4noc(=O)[nH]4)C[C@@H]2C)CC3)cc(C)c1F] | 2-((S)-2-(4-fluoro-3,5- dimethylphenyl)- 4-methyl-5-(1- ((1S,2S)-2-methyl- 1-(5-oxo-4,5- dihydro-1,2,4-oxadiazol-3- yl)cyclopropyl)- 5-(tetrahydro-2H- pyran-4-yl)-1H-indole-2- carbonyl)-4,5,6,7- tetrahydro-2H- pyrazolo[4,3-c] pyridin-3-yl)-N-(1- isopropyl-1H-indazol-5- yl)acetamide | 840.0 |
| 86 | 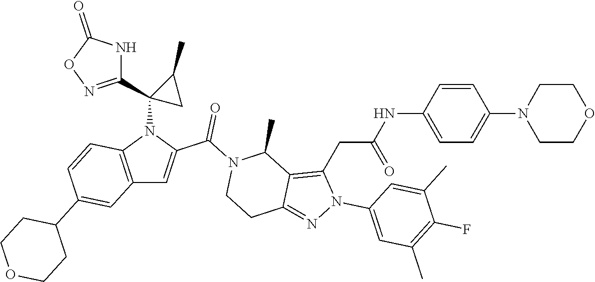 [SMILES: Cc1cc(-n2nc3c(c2CC(=O)Nc2ccc(N4CCOCC4)cc2)[C@H](C)N(C(=O)c2cc4cc(C5CCOCC5)ccc4n2[C@@]2(c4noc(=O)[nH]4)C[C@@H]2C)CC3)cc(C)c1F] | 2-((S)-2-(4-fluoro-3,5- dimethylphenyl)- 4-methyl-5-(1- ((1S,2S)-2-methyl- 1-(5-oxo-4,5- dihydro-1,2,4-oxadiazol-3- yl)cyclopropyl)- 5-(tetrahydro-2H- pyran-4-yl)-1H-indole-2- carbonyl)-4,5,6,7- tetrahydro-2H- pyrazolo[4,3-c] pyridin-3-yl)-N-(4- morpholinophenyl) acetamide | 843.4 |
| 87 | 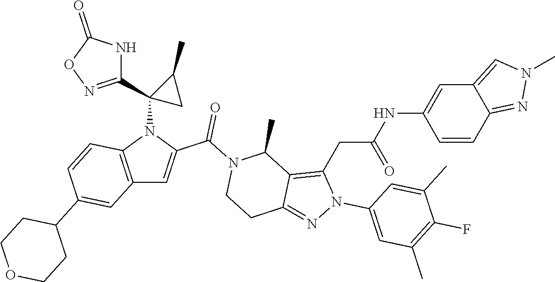 [SMILES: Cc1cc(-n2nc3c(c2CC(=O)Nc2ccc4nn(C)cc4c2)[C@H](C)N(C(=O)c2cc4cc(C5CCOCC5)ccc4n2[C@@]2(c4noc(=O)[nH]4)C[C@@H]2C)CC3)cc(C)c1F] | 2-((S)-2-(4-fluoro-3,5- dimethylphenyl)- 4-methyl-5-(1- ((1S,2S)-2-methyl- 1-(5-oxo-4,5- dihydro-1,2,4-oxadiazol-3- yl)cyclopropyl)- 5-(tetrahydro-2H- pyran-4-yl)-1H-indole-2- carbonyl)-4,5,6,7- tetrahydro-2H- pyrazolo[4,3-c] pyridin-3-yl)-N-(2- methyl-2H-indazol- 5-yl)acetamide | 812.4 |
| 88 | 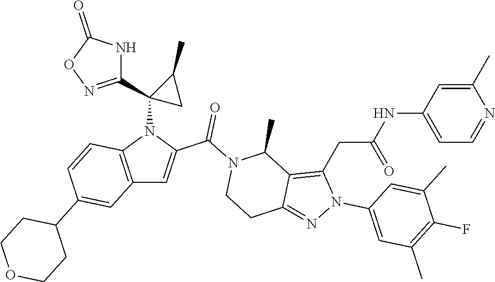 [SMILES: Cc1cc(NC(=O)Cc2c3c(nn2-c2cc(C)c(F)c(C)c2)CCN(C(=O)c2cc4cc(C5CCOCC5)ccc4n2[C@@]2(c4noc(=O)[nH]4)C[C@@H]2C)[C@H]3C)ccn1] | 2-((S)-2-(4-fluoro-3,5- dimethylphenyl)- 4-methyl-5-(1- ((1S,2S)-2-methyl- 1-(5-oxo-4,5- dihydro-1,2,4-oxadiazol- 3-yl)cyclopropyl)- 5-(tetrahydro-2H- pyran-4-yl)-1H-indole-2- carbonyl)-4,5,6,7- tetrahydro-2H- pyrazolo[4,3-c] pyridin-3-yl)-N-(2- methylpyridin-4-yl) acetamide | 773.4 |
| 89 | 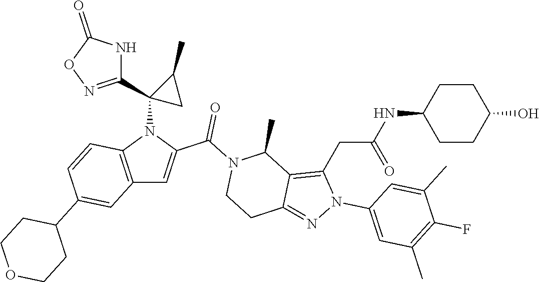 [SMILES: Cc1cc(-n2nc3c(c2CC(=O)N[C@H]2CC[C@H](O)CC2)[C@H](C)N(C(=O)c2cc4cc(C5CCOCC5)ccc4n2[C@@]2(c4noc(=O)[nH]4)C[C@@H]2C)CC3)cc(C)c1F] | 2-((S)-2-(4-fluoro-3,5- dimethylphenyl)- 4-methyl-5-(1- ((1S,2S)-2-methyl- 1-(5-oxo-4,5- dihydro-1,2,4-oxadiazol- 3-yl)cyclopropyl)- 5-(tetrahydro-2H- pyran-4-yl)-1H-indole-2- carbonyl)-4,5,6,7- tetrahydro-2H- pyrazolo[4,3-c] pyridin-3-yl)-N- ((1r,4S)-4- hydroxycyclohexyl) acetamide | 780.4 |
| 90 | 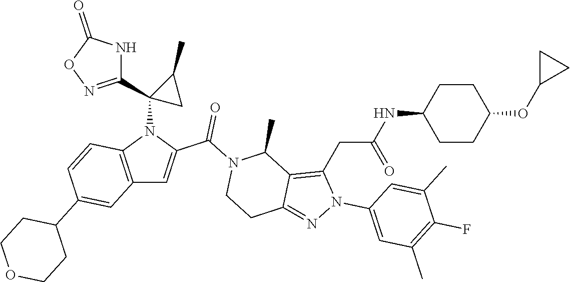 [SMILES: Cc1cc(-n2nc3c(c2CC(=O)N[C@H]2CC[C@H](OC4CC4)CC2)[C@H](C)N(C(=O)c2cc4cc(C5CCOCC5)ccc4n2[C@@]2(c4noc(=O)[nH]4)C[C@@H]2C)CC3)cc(C)c1F] | N-((1r,4S)-4- cyclopropoxycyclohexyl)- 2-((S)-2- (4-fluoro-3,5- dimethylphenyl)-4- methyl-5-(1-((1S,2S)- 2-methyl-1- (5-oxo-4,5-dihydro-1,2,4- oxadiazol-3-yl) cyclopropyl)-5- (tetrahydro-2H- pyran-4-yl)-1H- indole-2- carbonyl)-4,5,6,7- tetrahydro-2H- pyrazolo[4,3- c]pyridin-3-yl)acetamide | 820.4 |
| 91 | 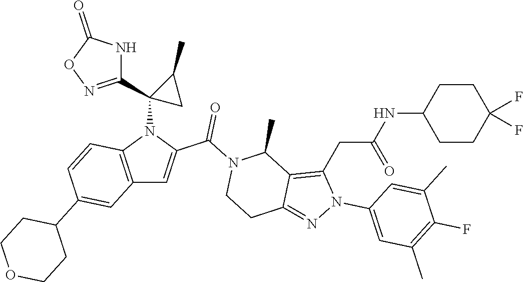 [SMILES: Cc1cc(-n2nc3c(c2CC(=O)NC2CCC(F)(F)CC2)[C@H](C)N(C(=O)c2cc4cc(C5CCOCC5)ccc4n2[C@@]2(c4noc(=O)[nH]4)C[C@@H]2C)CC3)cc(C)c1F] | N-(4,4-difluorocyclohexyl)- 2-((S)- 2-(4-fluoro-3,5- dimethylphenyl)-4- methyl-5-(1-((1S,2S)- 2-methyl-1- (5-oxo-4,5-dihydro-1,2,4- oxadiazol-3-yl) cyclopropyl)-5- (tetrahydro-2H- pyran-4-yl)-1H- indole-2- carbonyl)-4,5,6,7- tetrahydro-2H- pyrazolo[4,3- c]pyridin-3-yl)acetamide | 800.7 |
| 92 | 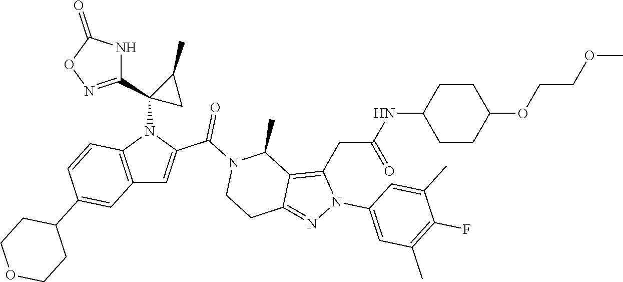 [SMILES: COCCOC1CCC(NC(=O)Cc2c3c(nn2-c2cc(C)c(F)c(C)c2)CCN(C(=O)c2cc4cc(C5CCOCC5)ccc4n2[C@@]2(c4noc(=O)[nH]4)C[C@@H]2C)[C@H]3C)CC1] | 2-((S)-2-(4-fluoro-3,5- dimethylphenyl)- 4-methyl-5-(1- ((1S,2S)-2-methyl- 1-(5-oxo-4,5- dihydro-1,2,4-oxadiazol-3- yl)cyclopropyl)- 5-(tetrahydro-2H- pyran-4-yl)-1H-indole-2- carbonyl)-4,5,6,7- tetrahydro-2H- pyrazolo[4,3- c]pyridin-3-yl)- N-(4-(2- methoxyethoxy) cyclohexyl)acetamide | 838.0 |
| 93 | 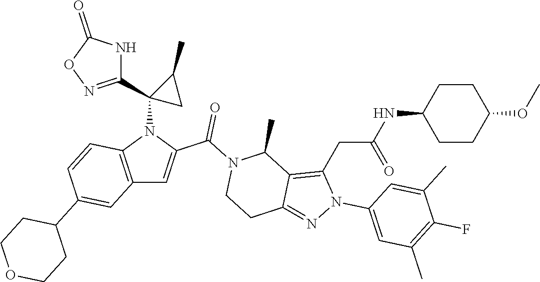 [SMILES: CO[C@H]1CC[C@H](NC(=O)Cc2c3c(nn2-c2cc(C)c(F)c(C)c2)CCN(C(=O)c2cc4cc(C5CCOCC5)ccc4n2[C@@]2(c4noc(=O)[nH]4)C[C@@H]2C)[C@H]3C)CC1] | 2-((S)-2-(4-fluoro-3,5- dimethylphenyl)- 4-methyl-5-(1- ((1S,2S)-2-methyl- 1-(5-oxo-4,5- dihydro-1,2,4-oxadiazol-3- yl)cyclopropyl)- 5-(tetrahydro-2H- pyran-4-yl)-1H-indole-2- carbonyl)-4,5,6,7- tetrahydro-2H- pyrazolo[4,3-c] pyridin-3-yl)-N- (trans-4- methoxycyclohexyl) acetamide | 794.4 |
| 94 | 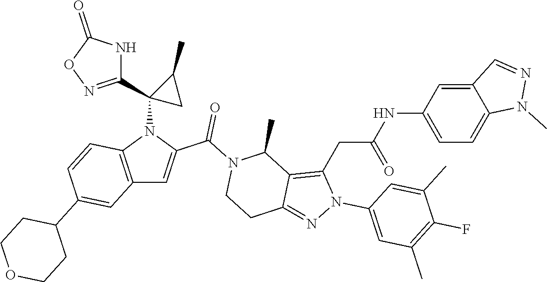 [SMILES: Cc1cc(-n2nc3c(c2CC(=O)Nc2ccc4c(cnn4C)c2)[C@H](C)N(C(=O)c2cc4cc(C5CCOCC5)ccc4n2[C@@]2(c4noc(=O)[nH]4)C[C@@H]2C)CC3)cc(C)c1F] | 2-((S)-2-(4-fluoro-3,5- dimethylphenyl)- 4-methyl-5-(1- ((1S,2S)-2-methyl- 1-(5-oxo-4,5- dihydro-1,2,4-oxadiazol- 3-yl)cyclopropyl)- 5-(tetrahydro-2H- pyran-4-yl)-1H-indole-2- carbonyl)-4,5,6,7- tetrahydro-2H- pyrazolo[4,3-c] pyridin-3-yl)-N-(1- methyl-1H-indazol- 5-yl)acetamide | 812.4 |
| 95 | 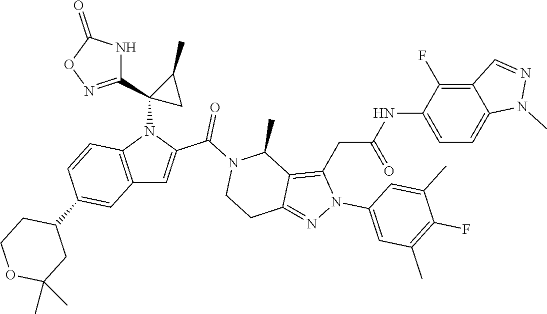 | 2-((4S)-5-(5-(2,2- dimethyltetrahydro- 2H-pyran-4- yl)-1-((1S,2S)-2- methyl-1-(5-oxo- 4,5-dihydro-1,2,4- oxadiazol-3- yl)cyclopropyl)- 1H-indole-2- carbonyl)-2-(4-fluoro-3,5- dimethylphenyl)- 4-methyl-4,5,6,7- tetrahydro-2H- pyrazolo[4,3- c]pyridin-3-yl)- N-(4-fluoro-1- methyl-1H-indazol- 5-yl)acetamide | 858.4 |
| 96 | 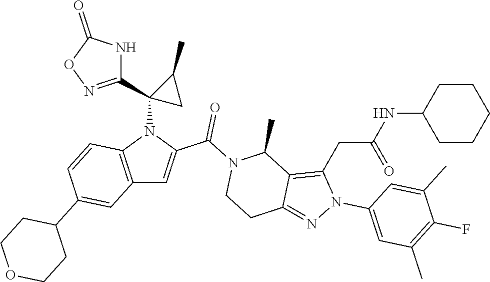 [SMILES: Cc1cc(-n2nc3c(c2CC(=O)NC2CCCCC2)[C@H](C)N(C(=O)c2cc4cc(C5CCOCC5)ccc4n2[C@@]2(c4noc(=O)[nH]4)C[C@@H]2C)CC3)cc(C)c1F] | N-cyclohexyl-2- ((S)-2-(4-fluoro- 3,5-dimethylphenyl)- 4-methyl-5- (1-((1S,2S)-2- methyl-1-(5-oxo- 4,5-dihydro-1,2,4- oxadiazol-3- yl)cyclopropyl)- 5-(tetrahydro-2H- pyran-4-yl)-1H-indole-2- carbonyl)-4,5,6,7- tetrahydro-2H- pyrazolo[4,3-c]pyridin-3- yl)acetamide | 764.7 |
| 97 | 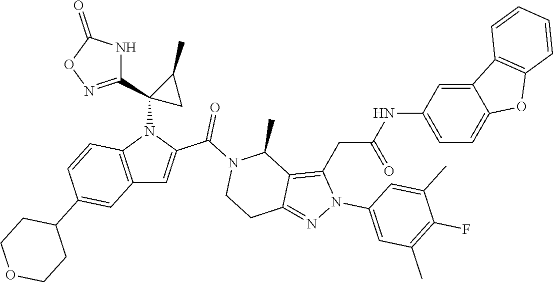 [SMILES: Cc1cc(-n2nc3c(c2CC(=O)Nc2ccc4oc5ccccc5c4c2)[C@H](C)N(C(=O)c2cc4cc(C5CCOCC5)ccc4n2[C@@]2(c4noc(=O)[nH]4)C[C@@H]2C)CC3)cc(C)c1F] | N-(dibenzo[b,d] furan-2-yl)-2-((S)- 2-(4-fluoro-3,5- dimethylphenyl)-4- methyl-5-(1- ((1S,2S)-2-methyl-1- (5-oxo-4,5-dihydro-1,2,4- oxadiazol-3-yl) cyclopropyl)-5- (tetrahydro-2H- pyran-4-yl)-1H- indole-2- carbonyl)-4,5,6,7- tetrahydro-2H- pyrazolo[4,3- c]pyridin-3-yl) acetamide | 848.7 |
| 98 | 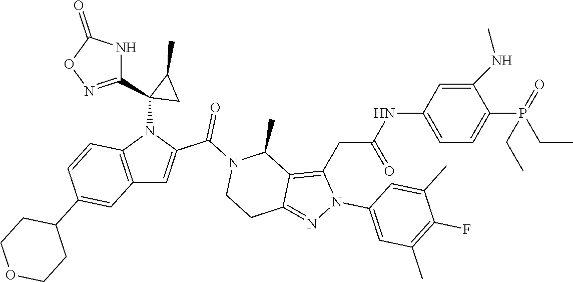 [SMILES: CCP(=O)(CC)c1ccc(NC(=O)Cc2c3c(nn2-c2cc(C)c(F)c(C)c2)CCN(C(=O)c2cc4cc(C5CCOCC5)ccc4n2[C@@]2(c4noc(=O)[nH]4)C[C@@H]2C)[C@H]3C)cc1NC] | N-(4-(diethylphosphoryl)- 3-(methylamino) phenyl)-2-((S)-2-(4- fluoro-3,5- dimethylphenyl)-4- methyl-5-(1- ((1S,2S)-2-methyl-1- (5-oxo-4,5-dihydro-1,2,4- oxadiazol-3-yl) cyclopropyl)-5- (tetrahydro-2H- pyran-4-yl)-1H- indole-2-carbonyl)-4,5,6,7- tetrahydro-2H- pyrazolo[4,3- c]pyridin-3-yl)acetamide | 891.3 |
| 99 | 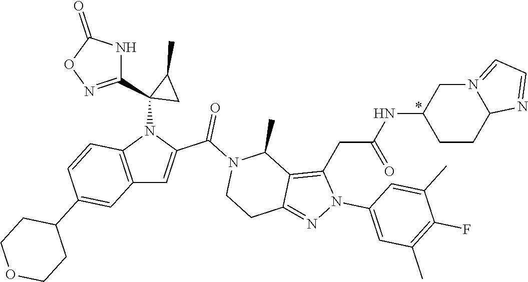 | 2-((S)-2-(4-fluoro-3,5- dimethylphenyl)- 4-methyl-5-(1- ((1S,2S)-2-methyl- 1-(5-oxo-4,5- dihydro-1,2,4-oxadiazol-3- yl)cyclopropyl)- 5-(tetrahydro-2H- pyran-4-yl)-1H-indole-2- carbonyl)-4,5,6,7- tetrahydro-2H- pyrazolo[4,3-c] pyridin-3-yl)-N- ((R)-4,5,6,7- tetrahydropyrazolo [1,5-a]pyridin- 5-yl)acetamide or 2-((S)-2-(4- fluoro-3,5- | 802.6 |
| | | dimethylphenyl)-4- | |
| | | methyl-5-(1- | |
| | | ((1S,2S)-2-methyl-1- | |
| | | (5-oxo-4,5-dihydro-1,2,4- | |
| | | oxadiazol-3-yl) | |
| | | cyclopropyl)-5- | |
| | | (tetrahydro-2H- | |
| | | pyran-4-yl)-1H- | |
| | | indole-2-carbonyl)-4,5,6,7- | |
| | | tetrahydro-2H-pyrazolo | |
| | | [4,3-c]pyridin-3-yl)- | |
| | | N-((S)-4,5,6,7- | |
| | | tetrahydropyrazolo | |
| | | [1,5-a]pyridin- | |
| | | 5-yl)acetamide | |
| 100 | 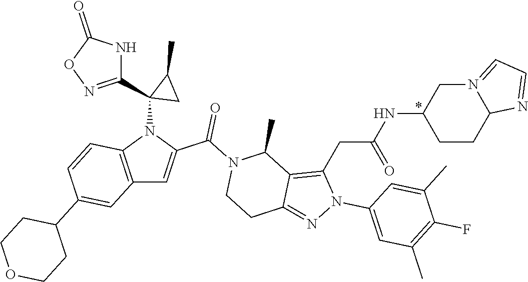 | 2-((S)-2-(4-fluoro-3,5- dimethylphenyl)- 4-methyl-5-(1- ((1S,2S)-2-methyl- 1-(5-oxo-4,5- dihydro-1,2,4-oxadiazol-3- yl)cyclopropyl)- 5-(tetrahydro-2H- pyran-4-yl)-1H-indole-2- carbonyl)-4,5,6,7- tetrahydro-2H- pyrazolo[4,3-c] pyridin-3-yl)-N- ((R)-4,5,6,7- tetrahydropyrazolo [1,5-a]pyridin- 5-yl)acetamide or 2-((S)-2-(4- | 802.6 |
| | | fluoro-3,5- | |
| | | dimethylphenyl)-4- | |
| | | methyl-5-(1- | |
| | | ((1S,2S)-2-methyl-1- | |
| | | (5-oxo-4,5-dihydro-1,2,4- | |
| | | oxadiazol-3-yl) | |
| | | cyclopropyl)-5- | |
| | | (tetrahydro-2H- | |
| | | pyran-4-yl)-1H- | |
| | | indole-2-carbonyl)-4,5,6,7- | |
| | | tetrahydro-2H-pyrazolo | |
| | | [4,3-c]pyridin-3-yl)- | |
| | | N-((S)-4,5,6,7- | |
| | | tetrahydropyrazolo | |
| | | [1,5-a]pyridin- | |
| | | 5-yl)acetamide | |
| 101 | 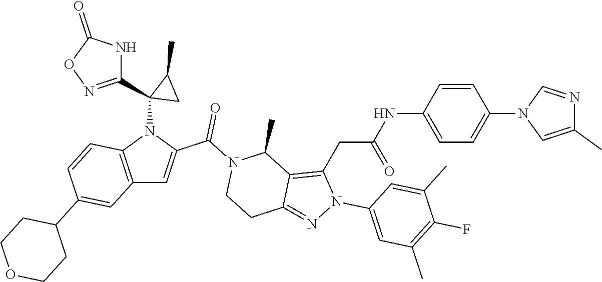 [SMILES: Cc1cn(-c2ccc(NC(=O)Cc3c4c(nn3-c3cc(C)c(F)c(C)c3)CCN(C(=O)c3cc5cc(C6CCOCC6)ccc5n3[C@@]3(c5noc(=O)[nH]5)C[C@@H]3C)[C@H]4C)cc2)cn1] | 2-((S)-2-(4-fluoro-3,5- dimethylphenyl)- 4-methyl-5-(1- ((1S,2S)-2-methyl- 1-(5-oxo-4,5- dihydro-1,2,4-oxadiazol-3- yl)cyclopropyl)- 5-(tetrahydro-2H- pyran-4-yl)-1H-indole-2- carbonyl)-4,5,6,7- tetrahydro-2H- pyrazolo[4,3-c] pyridin-3-yl)-N-(4- (4-methyl-1H-imidazol-1- yl)phenyl)acetamide | 838.4 |
| 199 | 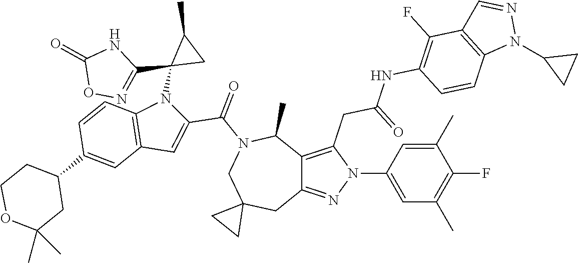 [SMILES: Cc1cc(-n2nc3c(c2CC(=O)Nc2ccc4c(cnn4C4CC4)c2F)[C@H](C)N(C(=O)c2cc4cc([C@H]5CCOC(C)(C)C5)ccc4n2[C@@]2(c4noc(=O)[nH]4)C[C@@H]2C)CC2(CC2)C3)cc(C)c1F] | N-(1-cyclopropyl- 4-fluoro- indazol-5-yl)-2- [(4S)-5-[5-[(4S)- 2,2-dimethyl- tetrahydropyran-4-yl]- 1-[(1S,2S)-2- methyl-1-(5-oxo-4H- 1,2,4-oxadiazol-3- yl)cyclopropyl] indole-2-carbonyl]- 2-(4-fluoro-3,5- dimethyl-phenyl)- 4-methyl-spiro[6, 8-dihydro-4H- pyrazolo[4,3-c] azepin-1-ium-7,1′- cyclopropane]-3-yl] acetamide formate | 925.4 |
| 200 | 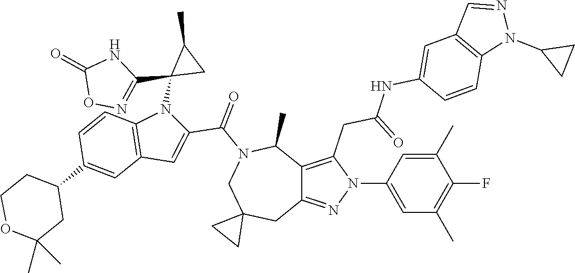 [SMILES: Cc1cc(-n2nc3c(c2CC(=O)Nc2ccc4c(cnn4C4CC4)c2)[C@H](C)N(C(=O)c2cc4cc([C@H]5CCOC(C)(C)C5)ccc4n2[C@@]2(c4noc(=O)[nH]4)C[C@@H]2C)CC2(CC2)C3)cc(C)c1F] | 2-[(4S)-5-[5-[(4S)-2,2- dimethyltetrahydropyran- 4-yl]-1- [(1S,2S)-2-methyl- 1-(5-oxo-4H- 1,2,4-oxadiazol-3- yl)cyclopropyl] indole-2-carbonyl]- 2-(4-fluoro-3,5- dimethyl-phenyl)- 4-methyl-spiro[6,8- dihydro-4H- pyrazolo[4,3-c] azepin-1-ium-7,1′- cyclopropane]-3-yl]-N-(1- methylindazol-5-yl) acetamide formate | 880.4 |
| 201 | 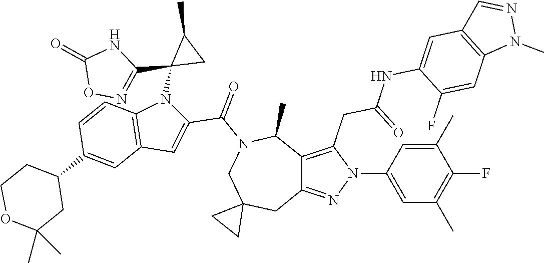 [SMILES: Cc1cc(-n2nc3c(c2CC(=O)Nc2cc4cnn(C)c4cc2F)[C@H](C)N(C(=O)c2cc4cc([C@H]5CCOC(C)(C)C5)ccc4n2[C@@]2(c4noc(=O)[nH]4)C[C@@H]2C)CC2(CC2)C3)cc(C)c1F] | 2-[(4S)-5-[5-[(4S)-2,2- dimethyltetrahydropyran- 4-yl]-1- [(1S,2S)-2-methyl- 1-(5-oxo-4H- 1,2,4-oxadiazol-3- yl)cyclopropyl] indole-2-carbonyl]- 2-(4-fluoro-3,5- dimethyl-phenyl)- 4-methyl-spiro[6,8- dihydro-4H- pyrazolo[4,3-c] azepin-1-ium-7,1′- cyclopropane]-3- yl]-N-(6-fluoro- 1-methyl-indazol-5-yl) acetamide formate | 898.4 |
| 202 | 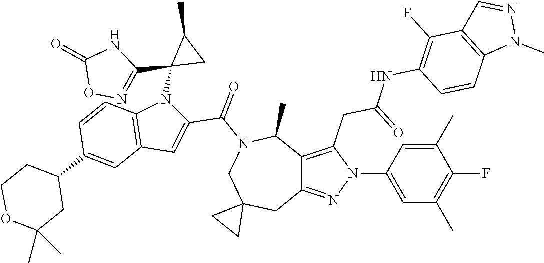 [SMILES: Cc1cc(-n2nc3c(c2CC(=O)Nc2ccc4c(cnn4C)c2F)[C@H](C)N(C(=O)c2cc4cc([C@H]5CCOC(C)(C)C5)ccc4n2[C@@]2(c4noc(=O)[nH]4)C[C@@H]2C)CC2(CC2)C3)cc(C)c1F] | 2-[(4S)-5-[5-[(4S)-2,2- dimethyltetrahydropyran- 4-yl]-1- [(1S,2S)-2-methyl- 1-(5-oxo-4H- 1,2,4-oxadiazol-3- yl)cyclopropyl] indole-2-carbonyl]- 2-(4-fluoro-3,5- dimethyl-phenyl)- 4-methyl-spiro[6,8- dihydro-4H- pyrazolo[4,3-c] azepin-1-ium-7,1′- cyclopropane]-3- yl]-N-(4-fluoro- 1-methyl-indazol-5-yl) acetamide formate | 898.4 |
| 203 | 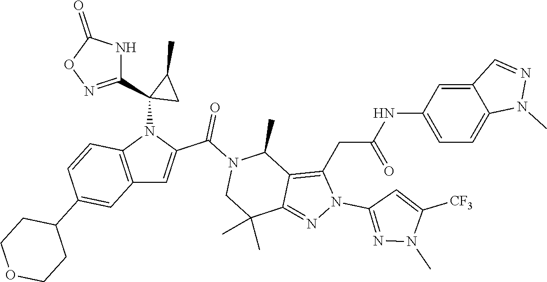 [SMILES: C[C@H]1c2c(nn(-c3cc(C(F)(F)F)n(C)n3)c2CC(=O)Nc2ccc3c(cnn3C)c2)C(C)(C)CN1C(=O)c1cc2cc(C3CCOCC3)ccc2n1[C@@]1(c2noc(=O)[nH]2)C[C@@H]1C] | N-(1-methyl-1H- indazol-5-yl)-2- ((S)-4,7,7-trimethyl- 5-(1-((1S,2S)- 2-methyl-1-(5-oxo- 4,5-dihydro- 1,2,4-oxadiazol-3- yl)cyclopropyl)- 5-(tetrahydro-2H- pyran-4-yl)-1H- indole-2-carbonyl)- 2-(1-methyl-5- (trifluoromethyl)- 1H-pyrazol-3- yl)-4,5,6,7-tetrahydro-2H- pyrazolo[4,3-c]pyridin-3- yl)acetamide, Formic Acid | 866.7 |
| 204 | 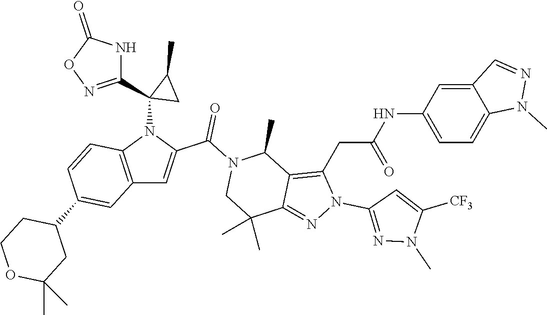 | 2-((S)-5-(5-((S)-2,2- dimethyltetrahydro- 2H-pyran-4- yl)-1-((1S,2S)-2- methyl-1-(5-oxo- 4,5-dihydro-1,2,4- oxadiazol-3-yl) cyclopropyl)-1H-indole- 2-carbonyl)-4,7,7- trimethyl-2-(1- methyl-5- (trifluoromethyl)-1H- pyrazol-3-yl)- 4,5,6,7-tetrahydro- 2H-pyrazolo[4,3- c]pyridin-3-yl)- N-(1-methyl-1H-indazol-5- yl)acetamide, Formic Acid | 894.5 |
| 205 | 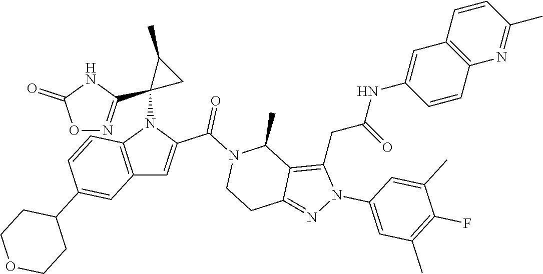 [SMILES: Cc1ccc2cc(NC(=O)Cc3c4c(nn3-c3cc(C)c(F)c(C)c3)CCN(C(=O)c3cc5cc(C6CCOCC6)ccc5n3[C@@]3(c5noc(=O)[nH]5)C[C@@H]3C)[C@H]4C)ccc2n1] | 2-((S)-2-(4-fluoro-3,5- dimethylphenyl)- 4-methyl-5-(1- ((1S,2S)-2-methyl- 1-(5-oxo-4,5- dihydro-1,2,4-oxadiazol-3- yl)cyclopropyl)- 5-(tetrahydro-2H- pyran-4-yl)-1H-indole-2- carbonyl)-4,5,6,7- tetrahydro-2H- pyrazolo[4,3-c] pyridin-3-yl)-N-(2- methylquinolin-6-yl) acetamide | 823.8 |
| 206 | 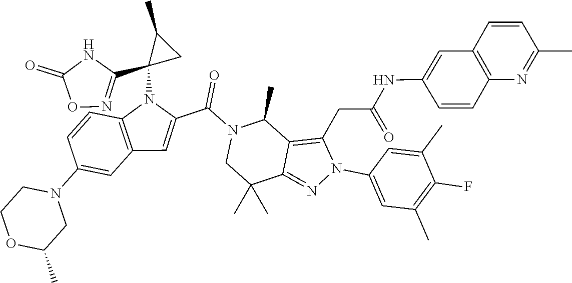 [SMILES: Cc1ccc2cc(NC(=O)Cc3c4c(nn3-c3cc(C)c(F)c(C)c3)C(C)(C)CN(C(=O)c3cc5cc(N6CCO[C@@H](C)C6)ccc5n3[C@@]3(c5noc(=O)[nH]5)C[C@@H]3C)[C@H]4C)ccc2n1] | 2-((S)-2-(4-fluoro-3,5- dimethylphenyl)- 4,7,7-trimethyl-5- (1-((1S,2S)-2- methyl-1-(5-oxo- 4,5-dihydro-1,2,4- oxadiazol-3- yl)cyclopropyl)-5-((S)-2- methylmorpholino)- 1H-indole-2- carbonyl)-4,5,6,7- tetrahydro-2H- pyrazolo[4,3-c] pyridin-3-yl)-N-(2- methylquinolin-6-yl) acetamide | 867.0 |
| 207 | 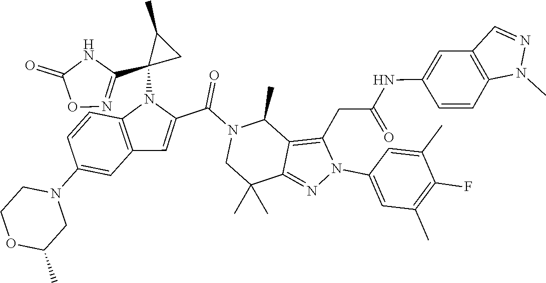 [SMILES: Cc1cc(-n2nc3c(c2CC(=O)Nc2ccc4c(cnn4C)c2)[C@H](C)N(C(=O)c2cc4cc(N5CCO[C@@H](C)C5)ccc4n2[C@@]2(c4noc(=O)[nH]4)C[C@@H]2C)CC3(C)C)cc(C)c1F] | 2-((S)-2-(4-fluoro-3,5- dimethylphenyl)- 4,7,7-trimethyl-5- (1-((1S,2S)-2-methyl- 1-(5-oxo- 4,5-dihydro-1,2,4- oxadiazol-3- yl)cyclopropyl)-5-((S)-2- methylmorpholino)- 1H-indole-2- carbonyl)-4,5,6,7- tetrahydro-2H- pyrazolo[4,3-c] pyridin-3-yl)-N-(1- methyl-1H-indazol- 5-yl)acetamide | 856.0 |
| 208 | 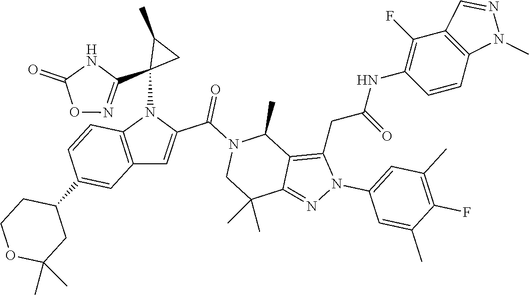 [SMILES: Cc1cc(-n2nc3c(c2CC(=O)Nc2ccc4c(cnn4C)c2F)[C@H](C)N(C(=O)c2cc4cc([C@H]5CCOC(C)(C)C5)ccc4n2[C@@]2(c4noc(=O)[nH]4)C[C@@H]2C)CC3(C)C)cc(C)c1F] | 2-((S)-5-(5-((S)-2,2- dimethyltetrahydro- 2H-pyran-4- yl)-1-((1S,2S)-2- methyl-1-(5-oxo- 4,5-dihydro-1,2,4- oxadiazol-3- yl)cyclopropyl)- 1H-indole-2- carbonyl)-2-(4-fluoro-3,5- dimethylphenyl)- 4,7,7-trimethyl- 4,5,6,7-tetrahydro-2H- pyrazolo[4,3-c] pyridin-3-yl)-N-(4- fluoro-1-methyl-1H- indazol-5-yl)acetamide | 886.5 |
| 209 | 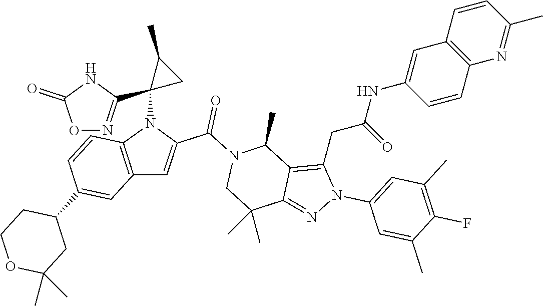 [SMILES: Cc1ccc2cc(NC(=O)Cc3c4c(nn3-c3cc(C)c(F)c(C)c3)C(C)(C)CN(C(=O)c3cc5cc([C@H]6CCOC(C)(C)C6)ccc5n3[C@@]3(c5noc(=O)[nH]5)C[C@@H]3C)[C@H]4C)ccc2n1] | 2-((S)-5-(5-((S)-2,2- dimethyltetrahydro- 2H-pyran-4- yl)-1-((1S,2S)-2- methyl-1-(5-oxo- 4,5-dihydro-1,2,4- oxadiazol-3- yl)cyclopropyl)- 1H-indole-2- carbonyl)-2-(4-fluoro- 3,5-dimethylphenyl)- 4,7,7-trimethyl- 4,5,6,7-tetrahydro-2H- pyrazolo[4,3-c] pyridin-3-yl)-N-(2- methylquinolin-6-yl) acetamide | 880.1 |
| 210 | 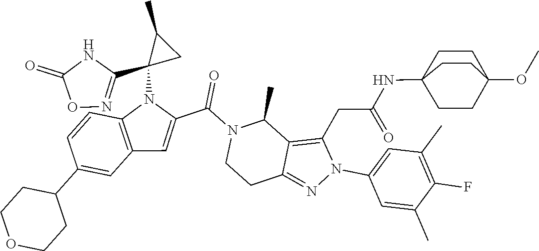 [SMILES: COC12CCC(NC(=O)Cc3c4c(nn3-c3cc(C)c(F)c(C)c3)CCN(C(=O)c3cc5cc(C6CCOCC6)ccc5n3[C@@]3(c5noc(=O)[nH]5)C[C@@H]3C)[C@H]4C)(CC1)CC2] | 2-((S)-2-(4-fluoro-3,5- dimethylphenyl)- 4-methyl-5-(1- ((1S,2S)-2-methyl- 1-(5-oxo-4,5- dihydro-1,2,4-oxadiazol-3- yl)cyclopropyl)- 5-(tetrahydro-2H- pyran-4-yl)-1H-indole-2- carbonyl)-4,5,6,7- tetrahydro-2H- pyrazolo[4,3-c] pyridin-3-yl)-N-(4- methoxybicyclo[2.2.2] octan-1-yl)acetamide | 820.4 |
| 211 | 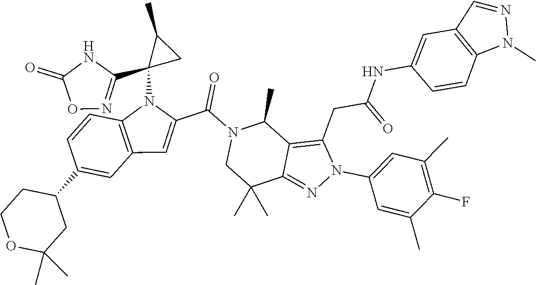 [SMILES: Cc1cc(-n2nc3c(c2CC(=O)Nc2ccc4c(cnn4C)c2)[C@H](C)N(C(=O)c2cc4cc([C@H]5CCOC(C)(C)C5)ccc4n2[C@@]2(c4noc(=O)[nH]4)C[C@@H]2C)CC3(C)C)cc(C)c1F] | 2-((S)-5-(5-((S)-2,2- dimethyltetrahydro- 2H-pyran-4- yl)-1-((1S,2S)-2- methyl-1-(5-oxo- 4,5-dihydro-1,2,4 -oxadiazol-3- yl)cyclopropyl)- 1H-indole-2- carbonyl)-2-(4-fluoro-3,5- dimethylphenyl)- 4,7,7-trimethyl- 4,5,6,7-tetrahydro-2H- pyrazolo[4,3-c] pyridin-3-yl)-N-(1- methyl-1H-indazol- 5-yl)acetamide | 869.1 |
| 212 | 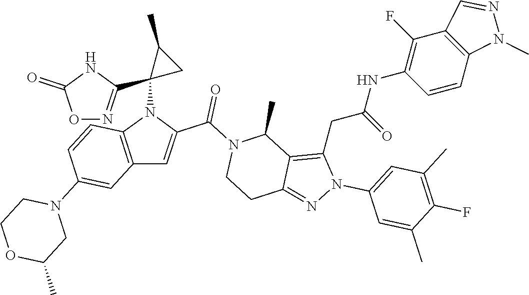 [SMILES: Cc1cc(-n2nc3c(c2CC(=O)Nc2ccc4c(cnn4C)c2F)[C@H](C)N(C(=O)c2cc4cc(N5CCO[C@@H](C)C5)ccc4n2[C@@]2(c4noc(=O)[nH]4)C[C@@H]2C)CC3)cc(C)c1F] | N-(4-fluoro-1-methyl- 1H-indazol- 5-yl)-2-((S)-2-(4-fluoro- 3,5-dimethylphenyl)- 4-methyl-5-(1- ((1S,2S)-2-methyl- 1-(5-oxo-4,5- dihydro-1,2,4-oxadiazol-3- yl)cyclopropyl)-5-((S)-2- methylmorpholino)- 1H-indole-2- carbonyl)-4,5,6,7- tetrahydro-2H- pyrazolo[4,3-c]pyridin- 3-yl)acetamide | 845.4 |
| 213 | 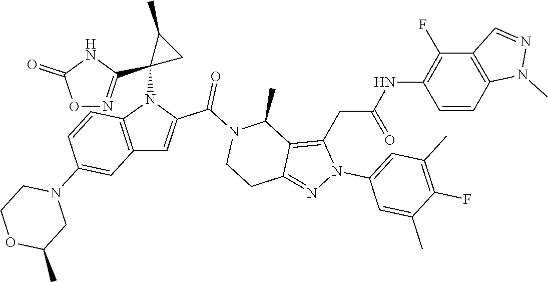 [SMILES: Cc1cc(-n2nc3c(c2CC(=O)Nc2ccc4c(cnn4C)c2F)[C@H](C)N(C(=O)c2cc4cc(N5CCO[C@H](C)C5)ccc4n2[C@@]2(c4noc(=O)[nH]4)C[C@@H]2C)CC3)cc(C)c1F] | N-(4-fluoro-1-methyl- 1H-indazol- 5-yl)-2-((S)-2-(4-fluoro- 3,5-dimethylphenyl)- 4-methyl-5- (1-((1S,2S)-2-methyl- 1-(5-oxo-4,5- dihydro-1,2,4-oxadiazol-3- yl)cyclopropyl)-5-((R)-2- methylmorpholino)- 1H-indole-2- carbonyl)-4,5,6,7- tetrahydro-2H- pyrazolo[4,3-c]pyridin- 3-yl)acetamide | 845.4 |
| 214 | 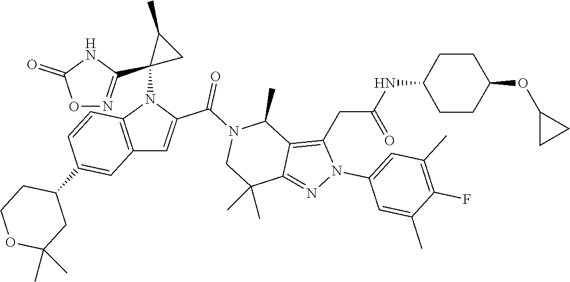 [SMILES: Cc1cc(-n2nc3c(c2CC(=O)N[C@H]2CC[C@H](OC4CC4)CC2)[C@H](C)N(C(=O)c2cc4cc([C@H]5CCOC(C)(C)C5)ccc4n2[C@@]2(c4noc(=O)[nH]4)C[C@@H]2C)CC3(C)C)cc(C)c1F] | N-((1r,4S)-4- cyclopropoxycyclohexyl)- 2-((S)-5- (5-((S)-2,2- dimethyltetrahydro- 2H-pyran-4-yl)-1-((1S,2S)- 2-methyl-1-(5-oxo- 4,5-dihydro- 1,2,4-oxadiazol-3- yl)cyclopropyl)- 1H-indole-2-carbonyl)-2-(4- fluoro-3,5- dimethylphenyl)-4,7,7- trimethyl-4,5,6,7- tetrahydro-2H- pyrazolo[4,3-c]pyridin-3- yl)acetamide | 877.1 |
| 331 Iso- mer A | 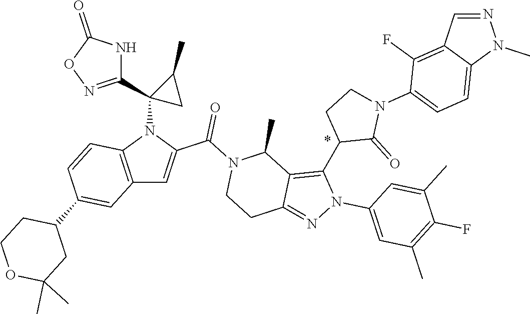 | 3-((1S,2S)-1-(5-((S)-2,2- dimethyltetrahydro- 2H-pyran-4- yl)-2-((S)-3-((R)- 1-(4-fluoro-1- methyl-1H-indazol-5-yl)-2- oxopyrrolidin-3- yl)-2-(4-fluoro- 3,5-dimethylphenyl)- 4-methyl- 4,5,6,7-tetrahydro-2H- pyrazolo[4,3-c]pyridine-5- carbonyl)-1H-indol-1-yl)-2- methylcyclopropyl)-1,2,4- oxadiazol-5(4H)-one or 3- ((1S,2S)-1-(5-((S)-2,2- dimethyltetrahydro- 2H-pyran-4- yl)-2-((S)-3-((S)- 1-(4-fluoro-1- | 884.4 |
| | | methyl-1H-indazol-5-yl)-2- | |
| | | oxopyrrolidin-3- | |
| | | yl)-2-(4-fluoro- | |
| | | 3,5-dimethylphenyl)- | |
| | | 4-methyl- | |
| | | 4,5,6,7-tetrahydro-2H- | |
| | | pyrazolo[4,3-c]pyridine-5- | |
| | | carbonyl)-1H-indol-1-yl)-2- | |
| | | methylcyclopropyl)-1,2,4- | |
| | | oxadiazol-5(4H)-one | |
| 331 Iso- mer B | 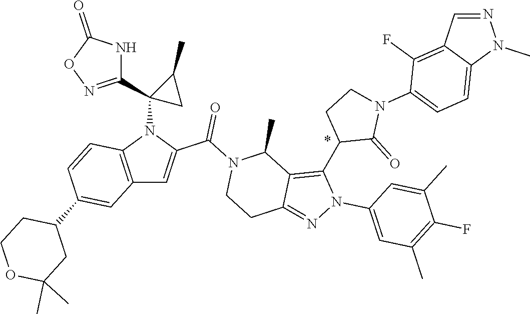 | 3-((1S,2S)-1-(5-((S)-2,2- dimethyltetrahydro- 2H-pyran-4-yl)-2-((S)- 3-((R)-1-(4-fluoro-1- methyl-1H-indazol-5-yl)- 2-oxopyrrolidin-3- yl)-2-(4-fluoro- 3,5-dimethylphenyl)- 4-methyl- 4,5,6,7-tetrahydro-2H- pyrazolo[4,3-c]pyridine-5- carbonyl)-1H-indol-1-yl)-2- methylcyclopropyl)-1,2,4- oxadiazol-5(4H)-one or 3- ((1S,2S)-1-(5-((S)-2,2- dimethyltetrahydro- 2H-pyran-4-yl)-2- ((S)-3-((S)-1-(4-fluoro-1- methyl-1H-indazol-5-yl)-2- | 884.4 |
| | | oxopyrrolidin-3- | |
| | | yl)-2-(4-fluoro-3,5- | |
| | | dimethylphenyl)-4-methyl- | |
| | | 4,5,6,7-tetrahydro-2H- | |
| | | pyrazolo[4,3-c]pyridine-5- | |
| | | carbonyl)-1H-indol-1-yl)-2- | |
| | | methylcyclopropyl)-1,2,4- | |
| | | oxadiazol-5(4H)-one | |
| 413 | 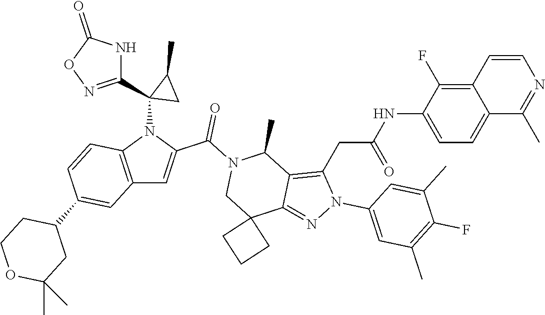 | 2-((S)-5′-(5-((S)-2,2- dimethyltetrahydro- 2H-pyran- 4-yl)-1-((1S,2S)-2- methyl-1-(5-oxo- 4,5-dihydro-1,2,4- oxadiazol-3-yl) cyclopropyl)-1H-indole-2- carbonyl)-2′-(4-fluoro-3,5- dimethylphenyl)-4′-methyl- 2′,4′,5′,6′- tetrahydrospiro [cyclobutane-1,7′- pyrazolo[4,3-c] pyridin]-3′-yl)-N- (5-fluoro-1- methylisoquinolin- 6-yl)acetamide | 909.4 |

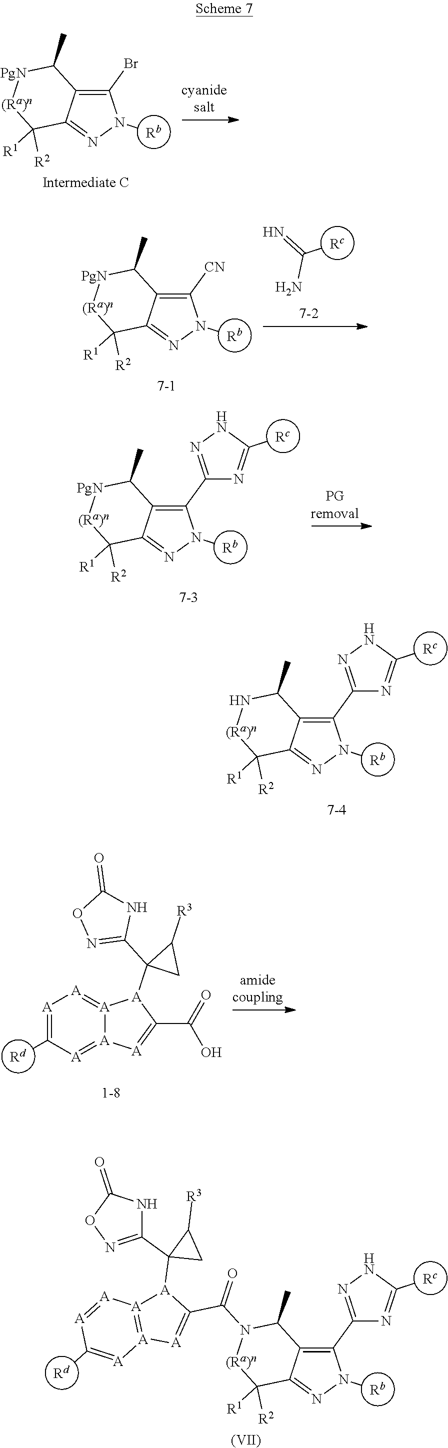

Compounds of formula (VII) are prepared from intermediate C by coupling with cyanide reagents such as K₄[Fe(CN)₆] via transition metal catalyzed cross-coupling conditions (e.g., Pd-catalysis) to provide nitrile 7-1. Treatment of 7-1 with amidines (7-2) in the presence of base (e.g., Cs₂CO₃) gives triazole 7-3. Removal of the protecting group (e.g., acidic conditions) of 7-3 provides amine 7-4 that is then coupled with carboxylic acid 1-8 to provide compounds of formula (VII).

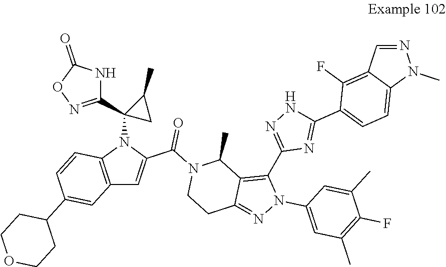 [SMILES: Cc1cc(-n2nc3c(c2-c2n[nH]c(-c4ccc5c(cnn5C)c4F)n2)[C@H](C)N(C(=O)c2cc4cc(C5CCOCC5)ccc4n2[C@@]2(c4noc(=O)[nH]4)C[C@@H]2C)CC3)cc(C)c1F]

### 3-((1S,2S)-1-(2-((S)-3-(5-(4-fluoro-1-methyl-1H-indazol-5-yl)-1H-1,2,4-triazol-3-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one (Scheme 7)

### Step 1: tert-butyl (S)-3-cyano-2-(4-fluoro-3,5-dimethylphenyl)-4-methyl-2,4,6,7-tetrahydro-5H-pyrazolo[4,3-c]pyridine-5-carboxylate

To a solution of tert-butyl (S)-3-bromo-2-(4-fluoro-3,5-dimethylphenyl)-4-methyl-2,4,6,7-tetrahydro-5H-pyrazolo[4,3-c]pyridine-5-carboxylate (500 mg, 1.14 mmol), K₄[Fe(CN)₆](210 mg, 0.570 mmol) and Na₂CO₃ (242 mg, 2.28 mmol) in NMP (8.0 mL) was added chloro(2-dicyclohexylphosphino-2′,4′,6′-triisopropyl-1,1′-biphenyl)[2-(2′-amino-1,1′-biphenyl)]palladium(II) (90 mg, 0.114 mmol) under an atmosphere of N₂. The resulting mixture was stirred at 100° C. for 16 h. Then, the mixture was quenched with H₂O (10 mL) and extracted with EtOAc (3×10 mL). The combined organic extracts were washed with brine (20 mL), dried over Na₂SO₄, filtered, and concentrated under reduced pressure. The crude residue was purified by prep-HPLC (67% to 87% MeCN/H₂O+0.1% TFA) to provide the title compound. MS (ESI) m/z 385.0.

### Step 2: tert-butyl (S)-3-(5-(4-fluoro-1-methyl-1H-indazol-5-yl)-1H-1,2,4-triazol-3-yl)-2-(4-fluoro-3,5-dimethylphenyl)-4-methyl-2,4,6,7-tetrahydro-5H-pyrazolo[4,3-c]pyridine-5-carboxylate

To a solution of tert-butyl (S)-3-cyano-2-(4-fluoro-3,5-dimethylphenyl)-4-methyl-2,4,6,7-tetrahydro-5H-pyrazolo[4,3-c]pyridine-5-carboxylate (56 mg, 0.15 mmol), 4-fluoro-1-methyl-1H-indazole-5-carboximidamide (28 mg, 0.15 mmol) and sodium carbonate (62 mg, 0.58 mmol) in toluene (2.0 mL) was added copper(II) acetate monohydrate (2.9 mg, 0.015 mmol), and the resulting mixture was stirred at 110° C. for 16 h. Then, the mixture was quenched with H₂O (10 mL) and extracted with EtOAc (3×10 mL). The combined organic extracts were dried over Na₂SO₄, filtered, and concentrated under reduced pressure. The crude residue was purified by pre-HPLC (57% to 77% MeCN/H₂O+0.1% TFA) to provide the title compound. MS (ESI) m/z 575.2.

### Step 4: (S)-3-(5-(4-fluoro-1-methyl-1H-indazol-5-yl)-1H-1,2,4-triazol-3-yl)-2-(4-fluoro-3,5-dimethylphenyl)-4-methyl-4,5,6,7-tetrahydro-2H-pyrazolo[4,3-c]pyridine

To a solution of tert-butyl (S)-3-(3-(4-fluoro-1-methyl-1H-indazol-5-yl)-1H-1,2,4-triazol-5-yl)-2-(4-fluoro-3,5-dimethylphenyl)-4-methyl-2,4,6,7-tetrahydro-5H-pyrazolo[4,3-c]pyridine-5-carboxylate (17 mg, 0.030 mmol) in dioxane (1.0 mL) was added 2M HCl/Dioxane (0.015 mL, 0.030 mmol), and the resulting mixture was stirred at 25° C. for 2 h. Then, the reaction mixture was concentrated under reduced pressure to provide title compound. MS (ESI) m/z 475.1.

### Step 5: 3-((1S,2S)-1-(2-((S)-3-(5-(4-fluoro-1-methyl-1H-indazol-5-yl)-1H-1,2,4-triazol-3-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one

To a solution of (S)-3-(3-(4-fluoro-1-methyl-1H-indazol-5-yl)-1H-1,2,4-triazol-5-yl)-2-(4-fluoro-3,5-dimethylphenyl)-4-methyl-4,5,6,7-tetrahydro-2H-pyrazolo[4,3-c]pyridine (14 mg, 0.030 mmol), 1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carboxylic acid (11 mg, 0.030 mmol), DIPEA (11 mg, 0.089 mmol) in DMF (1.0 mL) was added HATU (17 mg, 0.044 mmol), and the resulting mixture was stirred at 25° C. for 16 h. Then, the reaction mixture was filtered, and purified by pre-HPLC (62% to 82% MeCN/H₂O+0.1% TFA) to provide the title compound as the TFA salt. ¹H NMR (400 MHz, chloroform-d) δ ppm 11.39-11.60 (m, 1H) 8.02-8.41 (m, 2H) 7.42-7.84 (m, 3H) 7.15-7.23 (m, 2H) 7.10 (d, J=6.20 Hz, 1H) 6.69-6.82 (m, 1H) 5.57-6.28 (m, 1H) 4.36-5.02 (m, 1H) 4.11-4.15 (m, 4H) 3.53-3.70 (m, 3H) 2.97-3.09 (m, 2H) 2.24-2.30 (m, 8H) 1.74-1.97 (m, 8H) 1.68 (d, J=6.44 Hz, 3H) 1.21-1.26 (m, 3H). MS (ESI) m/z 840.3. The following examples in table 7 were prepared according to scheme 7 using the procedures outlined in the synthesis of example 102.

TABLE 7

| | | | MS |
|---|---|---|---|
| Ex. | Structure | Name | (M + 1) |
| 103 | 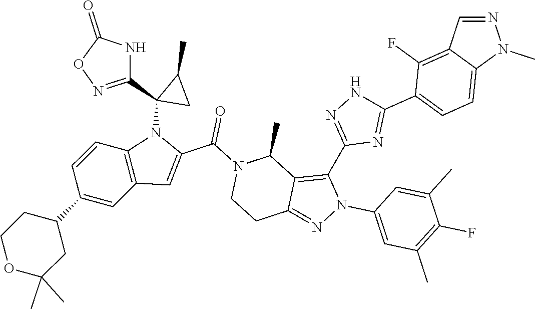 | 3-((1S,2S)-1-(5-((S)-2,2- dimethyltetrahydro-2H- pyran-4-yl)-2-((S)-3-(5-(4- fluoro-1-methyl-1H- indazol-5-yl)-1H-1,2,4- triazol-3-yl)-2-(4-fluoro- 3,5-dimethylphenyl)-4- methyl-4,5,6,7-tetrahydro- 2H-pyrazolo[4,3- c]pyridine-5-carbonyl)- 1H-indol-1-yl)-2- methylcyclopropyl)-1,2,4- oxadiazol-5(4H)-one | 868.3 |
| 104 | 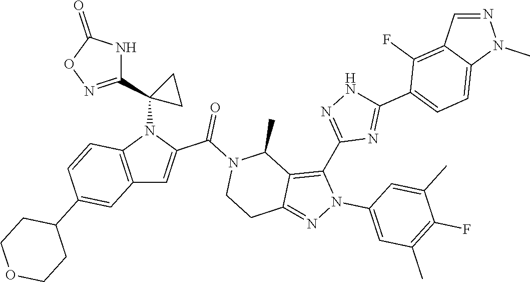 | (S)-3-(1-(2-(3-(5-(4- fluoro-1-methyl-1H- indazol-5-yl)-1H-1,2,4- triazol-3-yl)-2-(4-fluoro- 3,5-dimethylphenyl)-4- methyl-4,5,6,7-tetrahydro- 2H-pyrazolo[4,3- c]pyridine-5-carbonyl)-5- (tetrahydro-2H-pyran-4- yl)-1H-indol-1- yl)cyclopropyl)-1,2,4- oxadiazol-5(4H)-one | 826.3 |

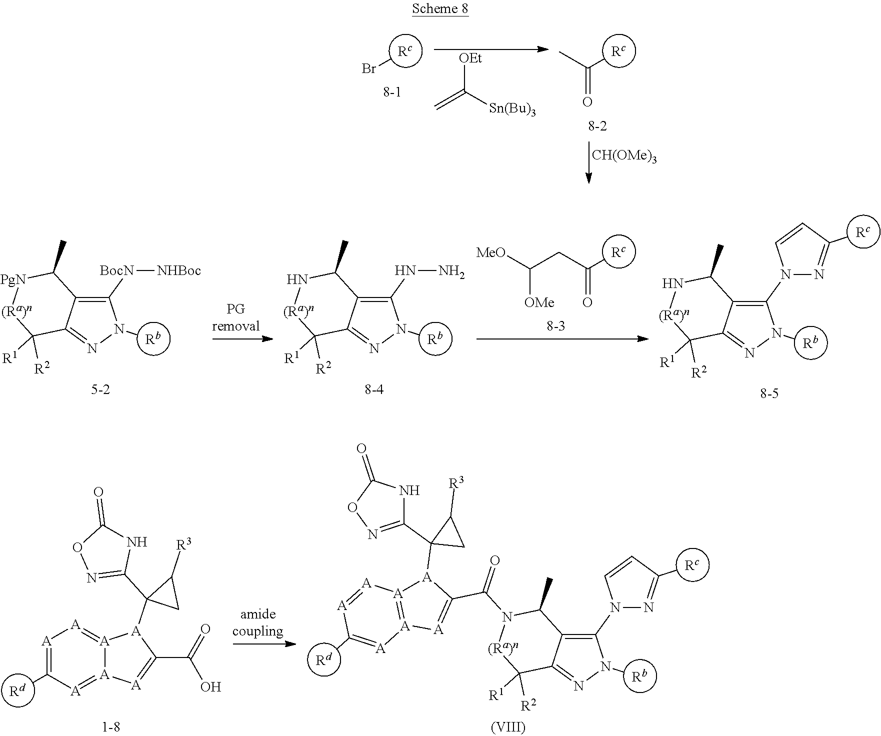

Compounds of formula (VIII) are prepared from intermediate 5-2 by removal of protecting groups (e.g., acidic conditions) to afford hydrazine 8-4. Then, 8-4 is condensed with ketone intermediates (8-3, two steps from 8-1 via Pd-catalyzed cross-coupling with tributyl(1-ethoxyvinyl)tin then condensation with trimethyl orthoformate) via acidic conditions (e.g., aqueous HCl) to provide pyrazole 8-5. Subsequently, 8-5 is coupled with carboxylic acid 1-8 to provide compounds of formula (VIII).

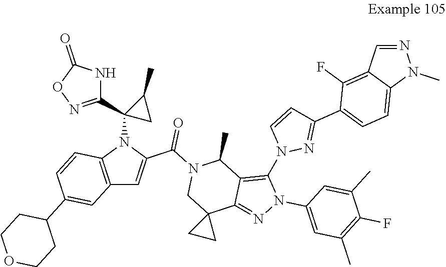 [SMILES: Cc1cc(-n2nc3c(c2-n2ccc(-c4ccc5c(cnn5C)c4F)n2)[C@H](C)N(C(=O)c2cc4cc(C5CCOCC5)ccc4n2[C@@]2(c4noc(=O)[nH]4)C[C@@H]2C)CC32CC2)cc(C)c1F]

### 3-((1S,2S)-1-(2-((S)-3′-(3-(4-fluoro-1-methyl-1H-indazol-5-yl)-1H-pyrazol-1-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one (Scheme 8)

### Step 1: 1-(4-fluoro-1-methyl-1H-indazol-5-yl)ethan-1-one

Tributyl(1-ethoxyvinyl)stannane (4.78 g, 13.2 mmol) and dichlorobis(triphenylphosphine)palladium(II) (0.306 g, 0.437 mmol) were added to 5-bromo-4-fluoro-1-methyl-1H-indazole (1.00 g, 4.37 mmol) in DMF (10.0 mL) at rt. The mixture was stirred at 80° C. for 16 h under an atmosphere of nitrogen. The mixture was cooled to rt, then diluted with EtOAc (50 mL) and treated with aq. KF solution (10 g of KF in 50 mL water). The mixture was stirred vigorously for 20 minutes. The resulting mixture was extracted with EtOAc (2×50 mL). The combined organic layers were stirred vigorously with 1M HCl (30 mL) for 16 h. The resulting mixture was extracted with EtOAc (3×50 mL), dried over Na₂SO₄, filtered, and concentrated under reduced pressure. The crude residue was purified by SiO₂ chromatography (0 to 33% EtOAc/Pet.ether) to provide the title compound. MS (ESI) m/z: 193.0 [M+H]+.

### Step 2: 1-(4-fluoro-1-methyl-1H-indazol-5-yl)-3,3-dimethoxypropan-1-one

Boron trifluoride etherate (0.31 mL, 2.5 mmol) was added drop-wise over 5 min to a solution of trimethoxymethane (0.23 mL, 2.1 mmol) in CH₂Cl₂ (5 mL) cooled to −40° C. The mixture was stirred for 10 min, then was transferred to an ice-water bath and stirred at 0° C. for 20 min. Then, the mixture was cooled to −78° C., and 1-(4-fluoro-1-methyl-1H-indazol-5-yl)ethan-1-one (200 mg, 1.0 mmol) was added followed by drop-wise addition of N-ethyl-N-isopropylpropan-2-amine (0.54 mL, 3.1 mmol) over 15 min. The resulting mixture was stirred for 16 h at 25° C., then poured onto a vigorously stirred mixture of sat. aq. NaHCO₃ solution (10 mL) and dichloromethane (10 mL). The organic phase was separated, washed with ice-cold 1M sulfuric acid solution (2×10 mL) and ice-cold water (2×10 mL), dried over anhydrous Na₂SO₄, filtered, and concentrated under reduced pressure provide the title compound. MS (ESI) m/z: 221.0 [M−OMe]+.

### Step 3: (S)-2′-(4-fluoro-3,5-dimethylphenyl)-3′-hydrazineyl-4′-methyl-2′,4′,5′,6′-tetrahydrospiro[cyclopropane-1,7′-pyrazolo[4,3-c]pyridine]

A solution of di-tert-butyl (S)-1-(5′-(tert-butoxycarbonyl)-2′-(4-fluoro-3,5-dimethylphenyl)-4′-methyl-2′,4′,5′,6′-tetrahydrospiro[cyclopropane-1,7′-pyrazolo[4,3-c]pyridin]-3′-yl)hydrazine-1,2-dicarboxylate (600 mg, 0.974 mmol) in 2 M HCl MeOH/MeOH (1:1) (6.0 mL) was stirred at 80° C. for 3 h, then cooled to rt and concentrated under reduced pressure to afford the title compound. MS (ESI) m/z: 316.4 [M+H]+.

### Step 4: (S)-3′-(3-(4-fluoro-1-methyl-1H-indazol-5-yl)-1H-pyrazol-1-yl)-2′-(4-fluoro-3,5-dimethylphenyl)-4′-methyl-2′,4′,5′,6′-tetrahydrospiro[cyclopropane-1,7′-pyrazolo[4,3-c]pyridine]

To a solution of (S)-2′-(4-fluoro-3,5-dimethylphenyl)-3′-hydrazineyl-4′-methyl-2′,4′,5′,6′-tetrahydrospiro[cyclopropane-1,7′-pyrazolo[4,3-c]pyridine](300 mg, 0.95 mmol) in MeOH/2M HCl MeOH (1:1) (6.0 mL) was added 1-(4-fluoro-1-methyl-1H-indazol-5-yl)-2,2-dimethoxyethan-1-one and (250 mg, 0.99 mmol). Then the mixture was stirred at 80° C. under an atmosphere of N₂ for 1 h. The reaction mixture was purified by reverse-phase HPLC (32% to 52% MeCN/water+0.1% TFA) to provide the undesired (first eluting regioisomer) product (S)-3′-(5-(4-fluoro-1-methyl-1H-indazol-5-yl)-1H-pyrazol-1-yl)-2′-(4-fluoro-3,5-dimethylphenyl)-4′-methyl-2′,4′,5′,6′-tetrahydrospiro[cyclopropane-1,7′-pyrazolo[4,3-c]pyridine](170 mg, 0.340 mmol) MS (ESI) m/z: 500.2 [M+H]+ and the title compound (second eluting regioisomer) MS (ESI) m/z: 500.2 [M+H]+.

### Step 5: 3-((1S,2S)-1-(2-((S)-3′-(3-(4-fluoro-1-methyl-1H-indazol-5-yl)-1H-pyrazol-1-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one

To a solution of 1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carboxylic acid (31 mg, 0.080 mmol) and (S)-3′-(3-(4-fluoro-1-methyl-1H-indazol-5-yl)-1H-pyrazol-1-yl)-2′-(4-fluoro-3,5-dimethylphenyl)-4′-methyl-2′,4′,5′,6′-tetrahydrospiro[cyclopropane-1,7′-pyrazolo[4,3-c]pyridine](40 mg, 0.080 mmol) in DMF (1.0 mL) was added DIPEA (0.042 mL, 0.24 mmol). The mixture was stirred at 20° C. for 10 min. Then, HATU (46 mg, 0.12 mmol) was added, and the resulting mixture was stirred at 20° C. for 16 h. The reaction mixture was purified by reverse-phase HPLC (80% to 100% MeCN/water+0.1% TFA) to provide the title compound. ¹H NMR (400 MHz, Acetonitrile-d3) δ=11.54-11.32 (m, 1H), 8.12-7.96 (m, 1H), 7.76-7.54 (m, 1H), 7.54-7.36 (m, 3H), 7.33-7.24 (m, 1H), 7.09-6.90 (m, 2H), 6.89-6.70 (m, 3H), 5.87-5.45 (m, 1H), 4.27-4.18 (m, 1H), 4.08-4.04 (m, 3H), 4.03-3.69 (m, 3H), 3.53-3.40 (m, 2H), 2.92-2.76 (m, 1H), 2.18-2.12 (m, 6H), 1.82-1.72 (m, 3H), 1.70-1.62 (m, 4H), 1.45 (br s, 4H), 1.41-1.21 (m, 2H), 0.88 (br s, 4H). The following examples in table 8 were prepared according to Scheme 8 using the procedures outlined in the synthesis of example 105.

TABLE 8

| | | | MS |
|---|---|---|---|
| Ex. | Structure | Name | (M + 1) |
| 106 | 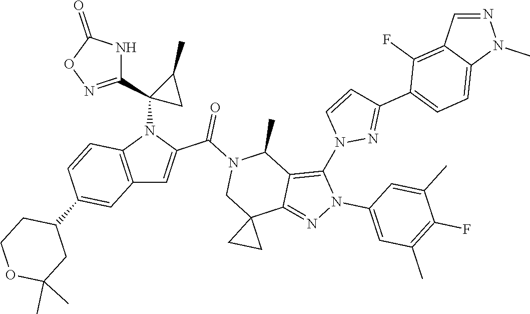 | 3-((1S,2S)-1-(5-((S)-2,2- dimethyltetrahydro-2H-pyran- 4-yl)-2-((S)-3′-(3-(4-fluoro-1- methyl-1H-indazol-5-yl)-1H- pyrazol-1-yl)-2′-(4-fluoro-3,5- dimethylphenyl)-4′-methyl- 2′,4′,5′,6′- tetrahydrospiro[cyclopropane- 1,7′-pyrazolo[4,3-c]pyridine]- 5′-carbonyl)-1H-indol-1-yl)-2- methylcyclopropyl)-1,2,4- oxadiazol-5(4H)-one | 893.3 |
| 107 | 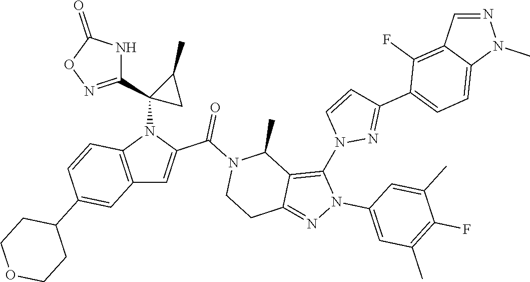 [SMILES: Cc1cc(-n2nc3c(c2-n2ccc(-c4ccc5c(cnn5C)c4F)n2)[C@H](C)N(C(=O)c2cc4cc(C5CCOCC5)ccc4n2[C@@]2(c4noc(=O)[nH]4)C[C@@H]2C)CC3)cc(C)c1F] | 3-((1S,2S)-1-(2-((S)-3-(3-(4- fluoro-1-methyl-1H-indazol-5- yl)-1H-pyrazol-1-yl)-2-(4- fluoro-3,5-dimethylphenyl)-4- methyl-4,5,6,7-tetrahydro-2H- pyrazolo[4,3-c]pyridine-5- carbonyl)-5-(tetrahydro-2H- pyran-4-yl)-1H-indol-1-yl)-2- methylcyclopropyl)-1,2,4- oxadiazol-5(4H)-one | 839.3 |
| 108 | 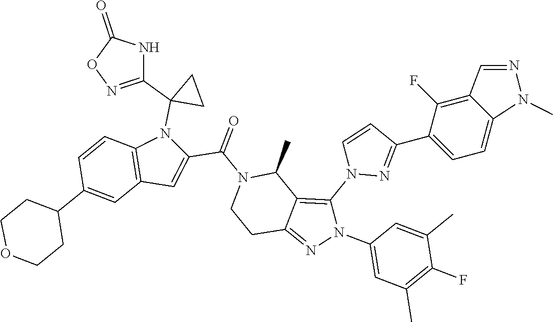 [SMILES: Cc1cc(-n2nc3c(c2-n2ccc(-c4ccc5c(cnn5C)c4F)n2)[C@H](C)N(C(=O)c2cc4cc(C5CCOCC5)ccc4n2C2(c4noc(=O)[nH]4)CC2)CC3)cc(C)c1F] | (S)-3-(1-(2-(3-(3-(4-fluoro-1- methyl-1H-indazol-5-yl)-1H- pyrazol-1-yl)-2-(4-fluoro-3,5- dimethylphenyl)-4-methyl- 4,5,6,7-tetrahydro-2H- pyrazolo[4,3-c]pyridine-5- carbonyl)-5-(tetrahydro-2H- pyran-4-yl)-1H-indol-1- yl)cyclopropyl)-1,2,4- oxadiazol-5(4H)-one | 825.3 |

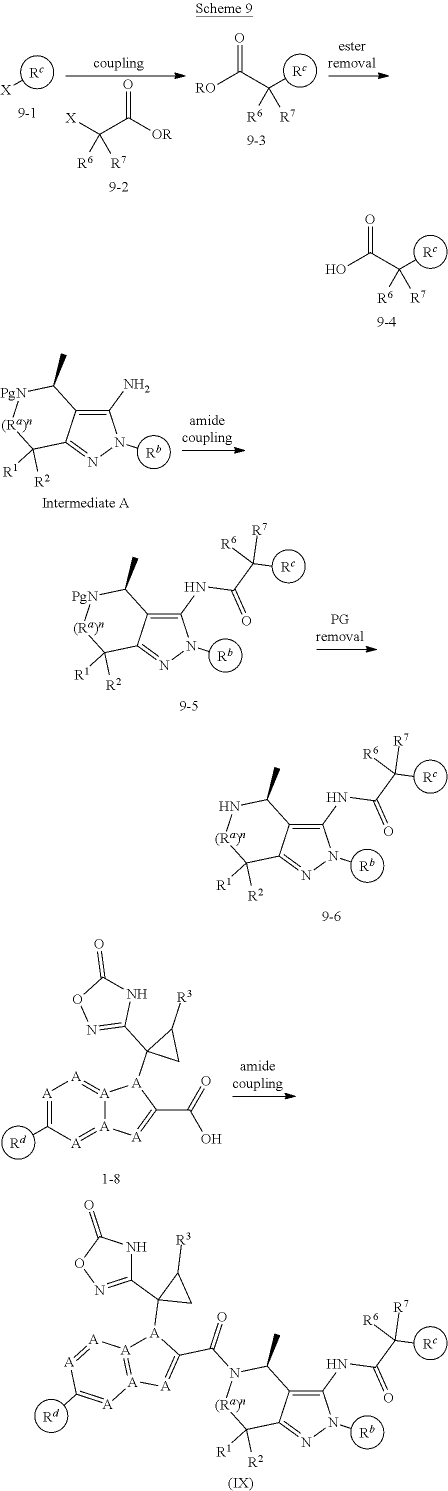

Compounds of formula (IX) are prepared from 9-1 (e.g., boronic acid or halide) by coupling with 9-2 (e.g., halide or zinc salt) via transition metal-catalyzed coupling conditions (e.g., Photoredox/Cu dual catalysis or Pd-catalysis) to provide ester 9-3. Ester removal (e.g., basic aqueous or acidic conditions) of 9-3 furnishes acid 9-4 that is then coupled with intermediate A. Removal of the protecting group (e.g., acidic conditions) of 9-5 provides amine 9-6 that is then coupled with carboxylic acid 1-8 to provide compounds of formula (IX).

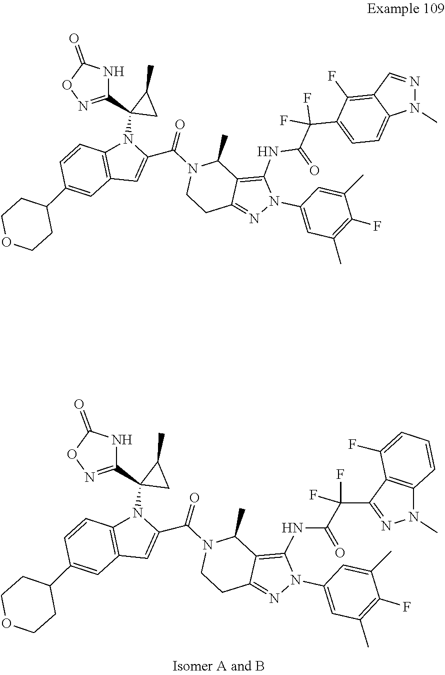 [SMILES: Cc1cc(-n2nc3c(c2NC(=O)C(F)(F)c2ccc4c(cnn4C)c2F)[C@H](C)N(C(=O)c2cc4cc(C5CCOCC5)ccc4n2[C@@]2(c4noc(=O)[nH]4)C[C@@H]2C)CC3)cc(C)c1F ; Cc1cc(-n2nc3c(c2NC(=O)C(F)(F)c2nn(C)c4cccc(F)c24)[C@H](C)N(C(=O)c2cc4cc(C5CCOCC5)ccc4n2[C@@]2(c4noc(=O)[nH]4)C[C@@H]2C)CC3)cc(C)c1F]

### 2,2-difluoro-2-(4-fluoro-1-methyl-1H-indazol-5-yl)-N—((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)acetamide and 2,2-difluoro-2-(4-fluoro-1-methyl-1H-indazol-3-yl)-N—((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)acetamide (scheme 9)

### Step 1: ethyl 2,2-difluoro-2-(4-fluoro-1-methyl-1H-indazol-5-yl)acetate and ethyl 2,2-difluoro-2-(4-fluoro-1-methyl-1H-indazol-3-yl)acetate

To a solution of copper(I) triflate toluene complex (96 mg, 0.19 mmol), (4-fluoro-1-methyl-1H-indazol-5-yl)boronic acid (290 mg, 1.5 mmol), dipotassium phosphate (260 mg, 1.5 mmol) and tris(2-phenylpyridine)iridium (9.7 mg, 0.015 mmol) was added ethyl bromodifluoroacetate (150 mg, 0.74 mmol) and DMF (6 mL) in an inert atmosphere glovebox. The mixture was stirred at 25° C. for 16 h under blue LEDs. The reaction was quenched with water (10 mL), and extracted with EtOAc (3×15 mL). The organic layers were combined, washed with brine (50 mL), dried over Na₂SO₄, filtered, and concentrated under reduced pressure. The crude residue was purified by SiO₂ chromatography (Pet. Et./EtOAc=2:1) to provide the title compounds. MS (ESI) m/z: 273.0, 273.0, [M+1]+.

### Step 2: 2,2-difluoro-2-(4-fluoro-1-methyl-1H-indazol-5-yl)acetic acid and 2,2-difluoro-2-(4-fluoro-1-methyl-1H-indazol-3-yl)acetic acid

To a solution of ethyl 2,2-difluoro-2-(4-fluoro-1-methyl-1H-indazol-5-yl)acetate and ethyl 2,2-difluoro-2-(4-fluoro-1-methyl-1H-indazol-3-yl)acetate (118 mg, 0.433 mmol) in THF (3 mL) and water (1 mL) was added sodium hydroxide (52.0 mg, 1.30 mmol). The reaction mixture was stirred for 3 h at 25° C., then concentrated under reduced pressure (to remove MeOH), and the pH was adjusted with HCl (pH<4). The resulting mixture was poured into water (5 mL) and extracted with EtOAc (3×5 mL). The organic layers were dried over Na₂SO₄, filtered, and concentrated under reduced pressure to provide the title compounds. MS (ESI) m/z: 244.9, 245.0, [M+1]+.

### Step 3: tert-butyl (S)-3-(2,2-difluoro-2-(4-fluoro-1-methyl-1H-indazol-5-yl)acetamido)-2-(4-fluoro-3,5-dimethylphenyl)-4-methyl-2,4,6,7-tetrahydro-5H-pyrazolo[4,3-c]pyridine-5-carboxylate and tert-butyl (S)-3-(2,2-difluoro-2-(4-fluoro-1-methyl-1H-indazol-3-yl)acetamido)-2-(4-fluoro-3,5-dimethylphenyl)-4-methyl-2,4,6,7-tetrahydro-5H-pyrazolo[4,3-c]pyridine-5-carboxylate

To a solution of 2,2-difluoro-2-(4-fluoro-1-methyl-1H-indazol-5-yl)acetic acid and 2,2-difluoro-2-(4-fluoro-1-methyl-1H-indazol-3-yl)acetic acid (35 mg, 0.14 mmol), tert-butyl (S)-3-amino-2-(4-fluoro-3,5-dimethylphenyl)-4-methyl-2,4,6,7-tetrahydro-5H-pyrazolo[4,3-c]pyridine-5-carboxylate (54 mg, 0.14 mmol), pyridine (0.12 mL, 1.4 mmol) in DCM (2.0 mL) was added POCl₃ (0.040 mL, 0.43 mmol) at 0° C. The mixture was stirred at 25° C. for 3 h. Then, the reaction mixture was diluted by water (5 mL) and extracted with DCM (3×8 mL). The organic layers were combined, dried over Na₂SO₄, filtered, and concentrated under reduced pressure. The crude mixture was purified by SiO₂ chromatography (Pet. Ether/EtOAc=2:1) to provide the title compounds. MS (ESI) m/z 601.1, 601.2 [M+1]+.

### Step 4: (S)-2,2-difluoro-2-(4-fluoro-1-methyl-1H-indazol-5-yl)-N-(2-(4-fluoro-3,5-dimethylphenyl)-4-methyl-4,5,6,7-tetrahydro-2H-pyrazolo[4,3-c]pyridin-3-yl)acetamide and (S)-2,2-difluoro-2-(4-fluoro-1-methyl-1H-indazol-3-yl)-N-(2-(4-fluoro-3,5-dimethylphenyl)-4-methyl-4,5,6,7-tetrahydro-2H-pyrazolo[4,3-c]pyridin-3-yl)acetamide

To a solution of tert-butyl (S)-3-(2,2-difluoro-2-(4-fluoro-1-methyl-1H-indazol-5-yl)acetamido)-2-(4-fluoro-3,5-dimethylphenyl)-4-methyl-2,4,6,7-tetrahydro-5H-pyrazolo[4,3-c]pyridine-5-carboxylate and tert-butyl (S)-3-(2,2-difluoro-2-(4-fluro-1-methyl-1H-indazol-3-yl)acetamido)-2-(4-fluoro-3,5-dimethylphenyl)-4-methyl-2,4,6,7-tetrahydro-5H-pyrazolo[4,3-c]pyridine-5-carboxylate (55 mg, 0.092 mmol) in dioxane (1 mL) was added 2M HCl/dioxane (0.046 mL, 0.092 mmol). The resulting mixture was stirred at 25° C. for 2 h then concentrated under reduced pressure to provide the title compounds. MS (ESI) m/z 501.2, 501.2 [M+1]+.

### Step 5: 2,2-difluoro-2-(4-fluoro-1-methyl-1H-indazol-5-yl)-N—((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)acetamide and 2,2-difluoro-2-(4-fluoro-1-methyl-1H-indazol-3-yl)-N—((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)acetamide

To a solution of (S)-2,2-difluoro-2-(4-fluoro-1-methyl-1H-indazol-5-yl)-N-(2-(4-fluoro-3,5-dimethylphenyl)-4-methyl-4,5,6,7-tetrahydro-2H-pyrazolo[4,3-c]pyridin-3-yl)acetamide and (S)-2,2-difluoro-2-(4-fluoro-1-methyl-1H-indazol-3-yl)-N-(2-(4-fluoro-3,5-dimethylphenyl)-4-methyl-4,5,6,7-tetrahydro-2H-pyrazolo[4,3-c]pyridin-3-yl)acetamide (45 mg, 0.090 mmol), 1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carboxylic acid (35 mg, 0.090 mmol), DIPEA (35 mg, 0.27 mmol) in DMF (1.0 mL) was added HATU (51 mg, 0.14 mmol), and the resulting mixture was stirred at 25° C. for 16 h. The reaction mixture was filtered, and the pH was adjusted with TFA (pH<7). Then, the crude mixture was purified by reverse-phase HPLC (60% to 80% MeCN/water+0.1% TFA) to provide the title compounds. Data for faster eluting regioisomer: ¹H NMR (400 MHz, acetonitrile-d3) δ ppm 11.36-11.54 (m, 1H), 8.77-9.16 (m, 1H), 7.94-8.19 (m, 1H), 7.50-7.59 (m, 2H), 7.42-7.49 (m, 1H), 7.31-7.41 (m, 1H), 7.31-7.41 (m, 1H), 7.23-7.30 (m, 1 H), 6.96-7.11 (m, 2H), 6.96-7.11 (m, 2H), 6.83 (d, J=2.74 Hz, 1H), 5.17-5.78 (m, 1H), 4.31-4.83 (m, 1H), 3.93-4.12 (m, 5H), 3.45-3.62 (m, 3H), 2.66-2.87 (m, 6H), 2.18 (d, J=1.79 Hz, 3H), 1.68-1.83 (m, 6H), 1.50-1.60 (m, 2H), 1.44 (d, J=6.68 Hz, 2H), 1.15 (d, J=6.20 Hz, 2H), 0.96 (d, J=5.84 Hz, 1H). MS (ESI) m/z 866.2. Data for slower eluting regioisomer: ¹H NMR (400 MHz, Acetonitrile-d3) δ ppm 11.33-11.63 (m, 1H), 8.72-9.13 (m, 1H), 7.49-7.56 (m, 2H), 7.35-7.47 (m, 2H), 7.18-7.31 (m, 2H), 6.75-7.13 (m, 3H), 5.23-5.88 (m, 1H), 4.36-4.80 (m, 1H), 4.15 (s, 2H), 3.94-4.00 (m, 3H), 3.43-3.66 (m, 3H), 2.63-3.23 (m, 9 H), 2.26 (d, J=1.55 Hz, 3H), 1.65-1.75 (m, 5H), 1.54-1.58 (m, 2H), 1.17 (d, J=6.08 Hz, 2H), 0.95 (br d, J=5.48 Hz, 1H). MS (ESI) m/z 866.2.

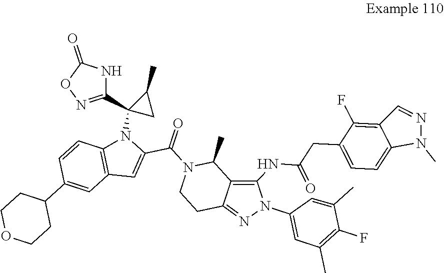 [SMILES: Cc1cc(-n2nc3c(c2NC(=O)Cc2ccc4c(cnn4C)c2F)[C@H](C)N(C(=O)c2cc4cc(C5CCOCC5)ccc4n2[C@@]2(c4noc(=O)[nH]4)C[C@@H]2C)CC3)cc(C)c1F]

### 2-(4-fluoro-1-methyl-1H-indazol-5-yl)-N—((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)acetamide (scheme 9)

### Step 1: tert-butyl 2-(4-fluoro-1-methyl-1H-indazol-5-yl)acetate

To a solution of Pd₂(dba)₃ (200 mg, 0.218 mmol), 5-bromo-4-fluoro-1-methyl-1H-indazole (500 mg, 2.18 mmol), 2-(dicyclohexylphosphino)-2′,4′,6′-triisopropyl-1,1′-biphenyl (208 mg, 0.437 mmol) in THF (8.0 mL) was added (2-(tert-butoxy)-2-oxoethyl)zinc(II) bromide (24.81 ml, 10.91 mmol), and the mixture was stirred at 75° C. for 16 h under an atmosphere of N₂. Then, the reaction mixture was quenched with H₂O (20 mL) and extracted with EtOAc (3×30 mL). The organic layers were combined, washed with brine (50 mL), dried over Na₂SO₄, filtered, and concentrated under reduced pressure. The crude residue was purified by silica gel chromatography (Pet.ether/EtOAc=3:1) to give the title compound. MS (ESI) m/z 265.0.

### Step 2: 2-(4-fluoro-1-methyl-1H-indazol-5-yl)acetic acid

To a solution of tert-butyl 2-(4-fluoro-1-methyl-1H-indazol-5-yl) acetate (770 mg, 2.91 mmol) in dioxane (10 mL) was added 2M HCl/dioxane (1.46 mL, 2.91 mmol), and the resulting mixture was stirred at 25° C. for 2 h. Then, the reaction mixture was concentrated under reduced pressure to provide the title compound. MS (ESI) m/z 209.0.

### Steps 3-5: 2-(4-fluoro-1-methyl-1H-indazol-5-yl)-N—((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)acetamide

The steps were performed in a similar fashion as described for example 110 to provide the title compound as the TFA salt. ¹H NMR (400 MHz, DMSO-d6) δ ppm 1.16 (br d, J=5.01 Hz, 3H) 1.38 (br d, J=6.32 Hz, 3H) 1.71 (br s, 6H) 2.20 (s, 9H) 2.78-2.88 (m, 2H) 3.80 (br s, 2H) 3.96 (br d, J=9.78 Hz, 3H) 4.01-4.09 (m, 4H) 4.20-4.66 (m, 1H) 5.09-5.59 (m, 1H) 6.89-6.94 (m, 1H) 7.17 (br d, J=5.96 Hz, 2H) 7.26 (br d, J=8.70 Hz, 1H) 7.34 (br d, J=7.51 Hz, 1H) 7.39 (br d, J=8.58 Hz, 1H) 7.44 (d, J=8.46 Hz, 1H) 7.49-7.58 (m, 1H) 8.03-8.13 (m, 1H) 10.14 (s, 1H) 11.74-11.82 (in, 1H). MS (ESI) m/z 830.3.

The following examples in table 9 were prepared according to scheme 9 using the procedures outlined in the synthesis of examples 109 or 110.

TABLE 9

| | | | MS |
|---|---|---|---|
| Example | Structure | Name | (M + 1) |
| 111 | 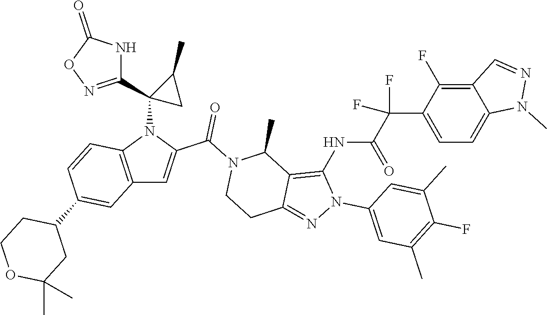 | N-((S)-5-(5-((S)-2,2- dimethyltetrahydro-2H- pyran-4-yl)-1-((1S,2S)-2- methyl-1-(5-oxo-4,5- dihydro-1,2,4-oxadiazol- 3-yl)cyclopropyl)-1H- indole-2-carbonyl)-2-(4- fluoro-3,5- dimethylphenyl)-4- methyl-4,5,6,7- tetrahydro-2H- pyrazolo[4,3-c]pyridin-3- yl)-2,2-difluoro-2-(4- fluoro-1-methyl-1H- indazol-5-yl)acetamide | 894.2 |
| 112 | 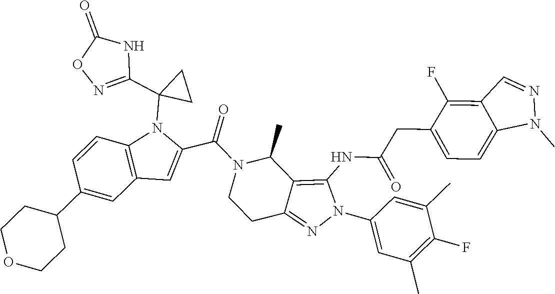 [SMILES: Cc1cc(-n2nc3c(c2NC(=O)Cc2ccc4c(cnn4C)c2F)[C@H](C)N(C(=O)c2cc4cc(C5CCOCC5)ccc4n2C2(c4noc(=O)[nH]4)CC2)CC3)cc(C)c1F] | (S)-2-(4-fluoro-1-methyl- 1H-indazol-5-yl)-N-(2- (4-fluoro-3,5- dimethylphenyl)-4- methyl-5-(1-(1-(5-oxo- 4,5-dihydro-1,2,4- oxadiazol-3- yl)cyclopropyl)-5- (tetrahydro-2H-pyran-4- yl)-1H-indole-2- carbonyl)-4,5,6,7- tetrahydro-2H- pyrazolo[4,3-c]pyridin-3- yl)acetamide | 816.3 |

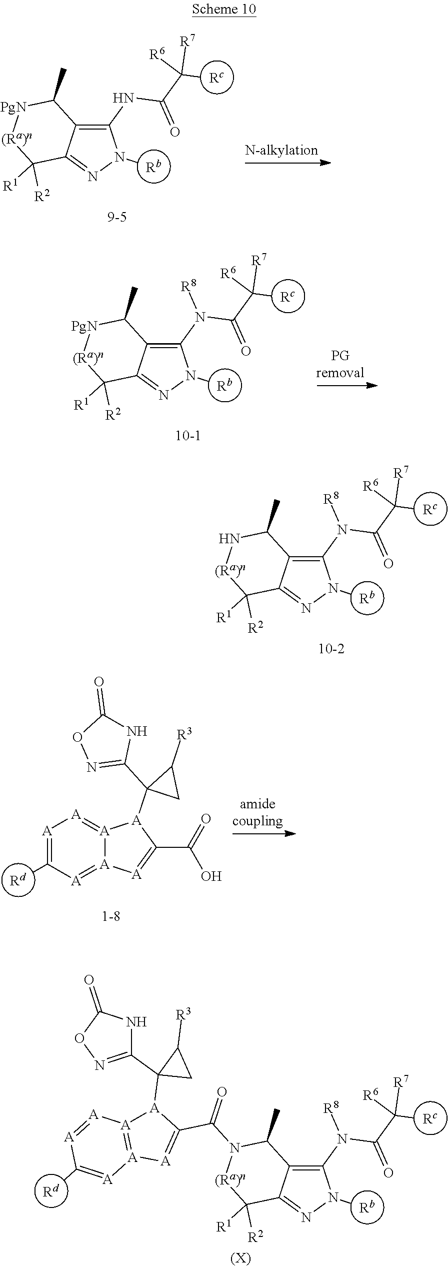

Compounds of formula (X) are prepared from 9-5 by N-alkylation (e.g., with alkyl halides under basic conditions) to provide 10-1. Removal of the protecting group (e.g., acidic conditions) of 10-1 provides amine 10-2 that is then coupled with carboxylic acid 1-8 to provide compounds of formula (X).

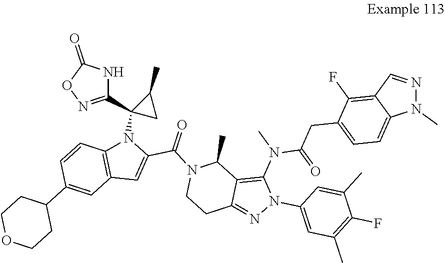 [SMILES: Cc1cc(-n2nc3c(c2N(C)C(=O)Cc2ccc4c(cnn4C)c2F)[C@H](C)N(C(=O)c2cc4cc(C5CCOCC5)ccc4n2[C@@]2(c4noc(=O)[nH]4)C[C@@H]2C)CC3)cc(C)c1F]

### 2-(4-fluoro-1-methyl-1H-indazol-5-yl)-N—((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N-methylacetamide (scheme 10)

### Step 1: tert-butyl (S)-3-(2-(4-fluoro-1-methyl-1H-indazol-5-yl)-N-methylacetamido)-2-(4-fluoro-3,5-dimethylphenyl)-4-methyl-2,4,6,7-tetrahydro-5H-pyrazolo[4,3-c]pyridine-5-carboxylate

To a stirring solution of tert-butyl (S)-3-(2-(4-fluoro-1-methyl-1H-indazol-5-yl)acetamido)-2-(4-fluoro-3,5-dimethylphenyl)-4-methyl-2,4,6,7-tetrahydro-5H-pyrazolo[4,3-c]pyridine-5-carboxylate (36 mg, 64 mol) and K₂CO₃ (26 mg, 0.19 mmol) in DMF (0.64 mL) was added Mel (2M in MTBE, 50 μL, 100 mol) at rt, and the mixture was stirred overnight. Then, EtOAc and water were added and the layers were separated. The aqueous layer was extracted with EtOAc (3 x) and the combined organic layers were dried with Na₂SO₄, filtered, and concentrated under reduced pressure. The crude mixture was purified by silica gel chromatography (0 to 100% EtOAc/Hex) to afford the title compound. MS (ESI) m/z 579.2 [M+H]+.

### Step 2: (S)-2-(4-fluoro-1-methyl-1H-indazol-5-yl)-N-(2-(4-fluoro-3,5-dimethylphenyl)-4-methyl-4,5,6,7-tetrahydro-2H-pyrazolo[4,3-c]pyridin-3-yl)-N-methylacetamide

Tert-butyl (S)-3-(2-(4-fluoro-1-methyl-1H-indazol-5-yl)-N-methylacetamido)-2-(4-fluoro-3,5-dimethylphenyl)-4-methyl-2,4,6,7-tetrahydro-5H-pyrazolo[4,3-c]pyridine-5-carboxylate (33 mg, 58 μmol) was dissolved in DCM (0.46 mL) and TFA (0.12 mL) at rt. The reaction was stirred at rt overnight. The reaction mixture was concentrated under reduced pressure, then EtOAc and sat. aq. Na₂CO₃ were added. The layers were separated, and the aqueous layer was extracted with EtOAc (3×). The combined organic layers were dried with Na₂SO₄, filtered, and concentrated under reduced pressure to produce the title compound. MS (ESI) m/z 479.2 [M+H]+.

### Step 3: 2-(4-fluoro-1-methyl-1H-indazol-5-yl)-N—((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N-methylacetamide

To a mixture of (S)-2-(4-fluoro-1-methyl-1H-indazol-5-yl)-N-(2-(4-fluoro-3,5-dimethylphenyl)-4-methyl-4,5,6,7-tetrahydro-2H-pyrazolo[4,3-c]pyridin-3-yl)-N-methylacetamide (28 mg, 58 mol), 1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carboxylic acid (26 mg, 69 mol), and HATU (26 mg, 69 μmol) in DMF (0.58 mL) was added DIPEA (50 μL, 290 mol) at rt. The reaction mixture was stirred at rt overnight. Then, the mixture was filtered and purified by prep-HPLC (70% to 99% MeCN/H2O+0.1% formic acid gradient) to provide the title compound. ¹H NMR (500 MHz, DMSO-d6) δ 11.74 (s, 1H), 8.20-7.96 (m, 1H), 7.66-7.04 (m, 7H), 6.96-6.70 (m, 1H), 5.76-5.27 (m, 1H), 4.71-4.23 (m, 1H), 4.09-3.93 (m, 6H), 3.54-3.43 (m, 3H), 3.15-2.80 (m, 6H), 2.28-2.22 (m, 6H), 1.84-1.59 (m, 8H), 1.50-1.37 (m, 2H), 1.30-1.03 (m, 4H). MS (ESI) m/z 844.4 [M+H]+.

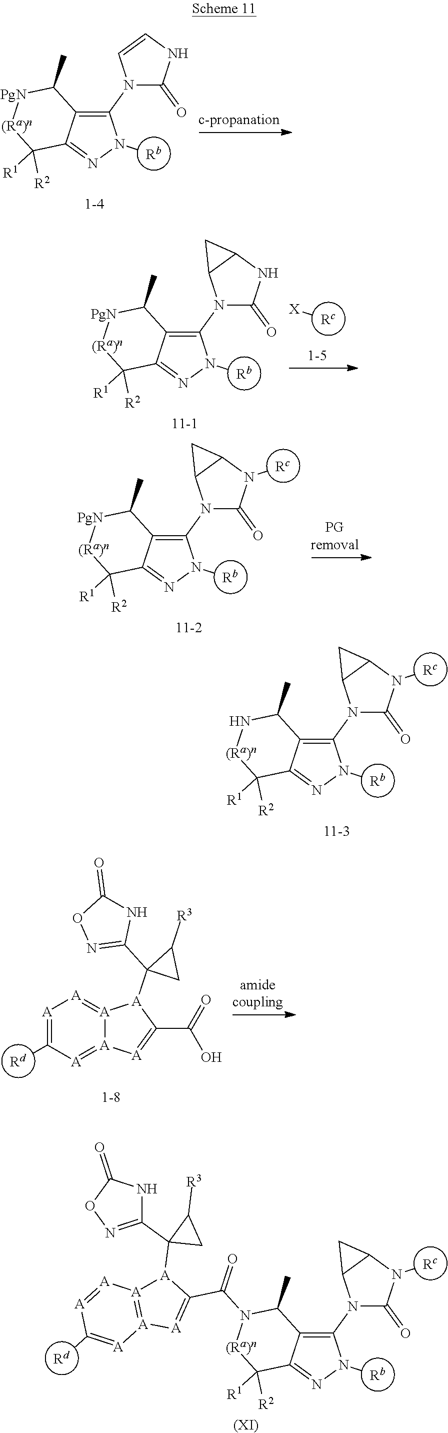

Compounds of formula (XI) are prepared from imidazol-2-one 1-4 by cyclopropanation (e.g., Fe-catalyzed carbene insertion conditions) to provide urea 11-1. Subsequently, 11-1 is coupled with halides or other partners such as boronic acids or sulfonates (1-5) via transition metal catalyzed cross-coupling (e.g., Pd—or Cu-catalysis) or alkylation (e.g., basic) conditions to furnish intermediate 11-3. Removal of the protecting group (e.g., acidic conditions) of 11-2 provides amine 11-3 that is then coupled with carboxylic acid 1-8 to provide compounds of formula (XI).

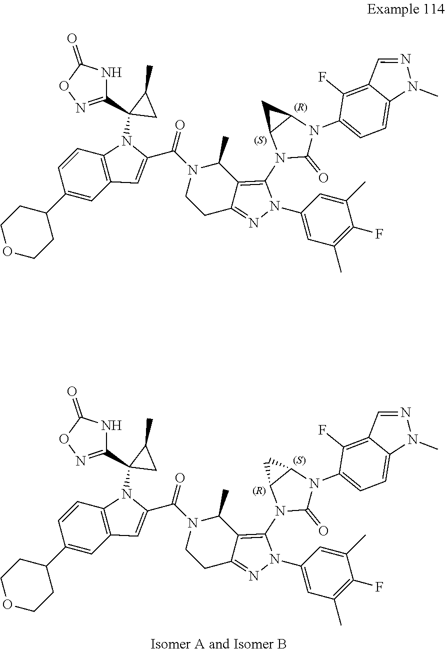

### 3-((1S,2S)-1-(2-((S)-3-((1R,5S)-4-(4-fluoro-1-methyl-1H-indazol-5-yl)-3-oxo-2,4-diazabicyclo[3.1.0]hexan-2-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one (scheme 11)

### Step 1: tert-butyl (S)-2-(4-fluoro-3,5-dimethylphenyl)-4-methyl-3-((1S,5R)-3-oxo-2,4-diazabicyclo[3.1.0]hexan-2-yl)-2,4,6,7-tetrahydro-5H-pyrazolo[4,3-c]pyridine-5-carboxylate and tert-butyl (S)-2-(4-fluoro-3,5-dimethylphenyl)-4-methyl-3-((1R,5S)-3-oxo-2,4-diazabicyclo[3.1.0]hexan-2-yl)-2,4,6,7-tetrahydro-5H-pyrazolo[4,3-c]pyridine-5-carboxylate

To an oven-dried vial were added a stir bar, tert-butyl (4S)-2-(4-fluoro-3,5-dimethylphenyl)-4-methyl-3-(2-oxo-2,3-dihydro-1H-imidazol-1-yl)-2,4,6,7-tetrahydro-5H-pyrazolo[4,3-c]pyridine-5-carboxylate (250 mg, 0.566 mmol), 5,10,15,20-tetraphenyl-21h,23h-porphine iron(III) (19.9 mg, 0.028 mmol), zinc (111 mg, 1.70 mmol) and lithium iodide (227 mg, 1.70 mmol). The vial was then capped and purged with argon. To the vial were then added anhydrous THF (5.6 mL) and anhydrous DCM (0.36 mL). The mixture was then stirred at 60° C. for 5 h. The reaction mixture was then quenched with methanol, concentrated under reduced pressure. The crude residue was purified sequentially via silica gel chromatography (1^{st} purification: 0-100% (3:1 EtOAc/EtOH) in hexanes then 2^{nd} purification: 40-80% (3:1 EtOAc/EtOH) in hexanes) then C18 reverse-phase chromatography (20-70% MeCN/water+0.1% formic acid modifier) to afford each of the two diastereomers of the title compound. Data for isomer A (faster eluting on C18 silica; contains minor amounts of isomer B as a contaminant): MS (ESI) m/z 456.4 [M+H]+. Data for Isomer B (slower eluting on C18 silica): MS (ESI) m/z 456.4 [M+H]+.

### Step 2: tert-butyl (S)-3-((1S,5R)-4-(4-fluoro-1-methyl-1H-indazol-5-yl)-3-oxo-2,4-diazabicyclo[3.1.0]hexan-2-yl)-2-(4-fluoro-3,5-dimethylphenyl)-4-methyl-2,4,6,7-tetrahydro-5H-pyrazolo[4,3-c]pyridine-5-carboxylate and tert-butyl (S)-3-((1R,5S)-4-(4-fluoro-1-methyl-1H-indazol-5-yl)-3-oxo-2,4-diazabicyclo[3.1.0]hexan-2-yl)-2-(4-fluoro-3,5-dimethylphenyl)-4-methyl-2,4,6,7-tetrahydro-5H-pyrazolo[4,3-c]pyridine-5-carboxylate

A mixture of tert-butyl (S)-2-(4-fluoro-3,5-dimethylphenyl)-4-methyl-3-((1S,5R)-3-oxo-2,4-diazabicyclo[3.1.0]hexan-2-yl)-2,4,6,7-tetrahydro-5H-pyrazolo[4,3-c]pyridine-5-carboxylate or tert-butyl (S)-2-(4-fluoro-3,5-dimethylphenyl)-4-methyl-3-((1R,5S)-3-oxo-2,4-diazabicyclo[3.1.0]hexan-2-yl)-2,4,6,7-tetrahydro-5H-pyrazolo[4,3-c]pyridine-5-carboxylate (isomer A, 18.0 mg, 0.040 mmol), copper(I) iodide (7.5 mg, 0.040 mmol), trans-N,N′-dimethylcyclohexane-1,2-diamine (11.2 mg, 12.5 μL, 0.079 mmol), 4-fluoro-5-iodo-1-methyl-1H-indazole (16.4 mg, 0.59 mmol) and potassium phosphate tribasic (25.2 mg, 0.119 mmol) in toluene (0.20 mL) was sparged with argon for 5 min. The mixture was then stirred under argon at 110° C. overnight. The mixture was then purified via silica gel chromatography (0-100% (3:1 EtOAc/EtOH) in hexanes) to afford isomer A of the title compound as a solid. MS (ESI) m/z 604.6 [M+H]+.

Isomer B of the title compound was prepared from isomer B of the starting material, in analogous fashion to isomer A. MS (ESI) m/z 604.6 [M+H]+.

### Step 3: (1S,5R)-2-(4-fluoro-1-methyl-1H-indazol-5-yl)-4-((S)-2-(4-fluoro-3,5-dimethylphenyl)-4-methyl-4,5,6,7-tetrahydro-2H-pyrazolo[4,3-c]pyridin-3-yl)-2,4-diazabicyclo[3.1.0]hexan-3-one and (1R,5S)-2-(4-fluoro-1-methyl-1H-indazol-5-yl)-4-((S)-2-(4-fluoro-3,5-dimethylphenvl)-4-methyl-4,5,6,7-tetrahydro-2H-pyrazolo[4,3-c]pyridin-3-yl)-2,4-diazabicyclo[3.1.0]hexan-3-one

A mixture of tert-butyl (S)-3-((1S,5R)-4-(4-fluoro-1-methyl-1H-indazol-5-yl)-3-oxo-2,4-diazabicyclo[3.1.0]hexan-2-yl)-2-(4-fluoro-3,5-dimethylphenyl)-4-methyl-2,4,6,7-tetrahydro-5H-pyrazolo[4,3-c]pyridine-5-carboxylate or tert-butyl (S)-3-((1R,5S)-4-(4-fluoro-1-methyl-1H-indazol-5-yl)-3-oxo-2,4-diazabicyclo[3.1.0]hexan-2-yl)-2-(4-fluoro-3,5-dimethylphenyl)-4-methyl-2,4,6,7-tetrahydro-5H-pyrazolo[4,3-c]pyridine-5-carboxylate (isomer A, 12.0 mg, 0.020 mmol) and 4M HCl/dioxane (0.20 mL, 0.80 mmol) was stirred at rt for 1 h. The mixture was the concentrated to provide crude isomer A of the title compound, which was used further without purification. MS (ESI) m/z 504.4 [M+H]+.

Isomer B of the title compound was prepared from isomer B of the starting material, in analogous fashion to isomer A. MS (ESI) m/z 504.6 [M+H]+.

### Step 4: 3-((1S,2S)-1-(2-((S)-3-((1S,5R)-4-(4-fluoro-1-methyl-1H-indazol-5-yl)-3-oxo-2,4-diazabicyclo[3.1.0]hexan-2-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one and 3-((1S,2S)-1-(2-((S)-3-((1R,5S)-4-(4-fluoro-1-methyl-1H-indazol-5-yl)-3-oxo-2,4-diazabicyclo[3.1.0]hexan-2-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one

To crude (1S,5R)-2-(4-fluoro-1-methyl-1H-indazol-5-yl)-4-((S)-2-(4-fluoro-3,5-dimethylphenyl)-4-methyl-4,5,6,7-tetrahydro-2H-pyrazolo[4,3-c]pyridin-3-yl)-2,4-diazabicyclo[3.1.0]hexan-3-one or (1R,5S)-2-(4-fluoro-1-methyl-1H-indazol-5-yl)-4-((S)-2-(4-fluoro-3,5-dimethylphenyl)-4-methyl-4,5,6,7-tetrahydro-2H-pyrazolo[4,3-c]pyridin-3-yl)-2,4-diazabicyclo[3.1.0]hexan-3-one (isomer A) from Step 3 were added 1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carboxylic acid (11.4 mg, 0.030 mmol), HATU (11.3 mg, 0.030 mmol) and DMF (0.13 mL). The mixture was then treated with DIPEA (25.7 mg, 34.6 μL, 0.20 mmol) and stirred at rt for 2 h. The mixture was then directly purified via C18 reverse-phase chromatography (10-100% MeCN/water+0.1% formic acid) to afford isomer A of the title compound as a solid. ¹H NMR (500 MHz, CD₃CN) δ 11.50 (s, 1H), 8.10-7.92 (m, 1H), 7.59-7.36 (m, 3H), 7.32-6.98 (m, 4H), 6.84 (s, 1H), 5.89-5.31 (m, 1H), 4.85-4.31 (m, 1H), 4.09-3.93 (m, 5H), 3.65-3.37 (m, 5H), 3.30-3.05 (m, 1H), 3.07-2.81 (m, 2H), 2.36-2.27 (m, 6H), 1.84-1.60 (m, 7H), 1.59-1.54 (m, 2H), 1.46-1.28 (m, 1H), 1.21-0.92 (m, 3H), 0.91-0.79 (m, 1H), 0.72-0.35 (m, 1H). MS (ESI) m/z 869.6.

Isomer B of the title compound was prepared from isomer B of the starting material, in analogous fashion to isomer A. ¹H NMR (500 MHz, CD₃CN) δ 11.54 (br s, 1H), 8.10-7.97 (m, 1H), 7.59-7.26 (m, 4H), 7.27-7.13 (m, 3H), 6.86-6.76 (m, 1H), 5.81-5.23 (m, 1H), 4.80-4.31 (m, 1H), 4.10-3.93 (m, 5H), 3.71-3.36 (m, 5H), 3.24-3.08 (m, 1H), 3.05-2.81 (m, 2H), 2.37-2.27 (m, 6H), 1.85-1.55 (m, 9H), 1.40-1.31 (m, 1H), 1.22-0.94 (m, 3H), 0.88-0.35 (in, 2H). MIS (ESI) m/z 869.8.

The following examples in table 10 were prepared according to scheme 11 using the procedures outlined in the synthesis of example 114 Isomer A and Isomer B.

TABLE 10

| | | | MS |
|---|---|---|---|
| Ex | Structure | Name | (M + 1) |
| 115 Isomer B | 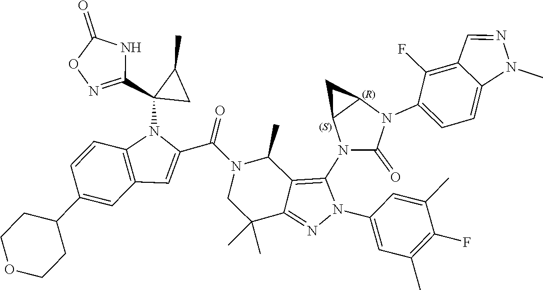 | 3-((1S,2S)-1-(2-((S)-3- ((1S,5R)-4-(4-fluoro-1- methyl-1H-indazol-5-yl)-3- oxo-2,4- diazabicyclo[3.1.0]hexan-2- yl)-2-(4-fluoro-3,5- dimethylphenyl)-4,7,7- trimethyl-4,5,6,7-tetrahydro- 2H-pyrazolo[4,3-c]pyridine-5- carbonyl)-5-(tetrahydro-2H- pyran-4-yl)-1H-indol-1-yl)-2- methylcyclopropyl)-1,2,4- oxadiazol-5(4H)-one or 3- ((1S,2S)-1-(2-((S)-3-((1R,5S)- 4-(4-fluoro-1-methyl-1H- indazol-5-yl)-3-oxo-2,4- diazabicyclo[3.1.0]hexan-2- yl)-2-(4-fluoro-3,5- dimethylphenyl)-4,7,7- | 897.6 |
| | | trimethyl-4,5,6,7-tetrahydro- | |
| | | 2H-pyrazolo[4,3-c]pyridine-5- | |
| | | carbonyl)-5-(tetrahydro-2H- | |
| | | pyran-4-yl)-1H-indol-1-yl)-2- | |
| | | methylcyclopropyl)-1,2,4- | |
| | | oxadiazol-5(4H)-one | |
| | or | | |
| | 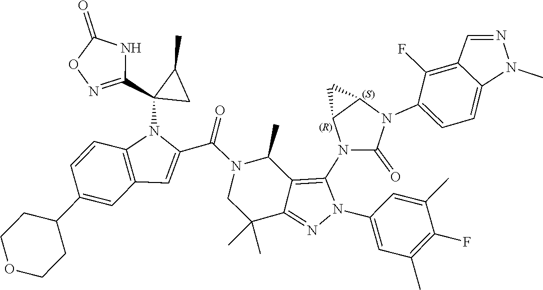 | | |
| 116 Isomer B | 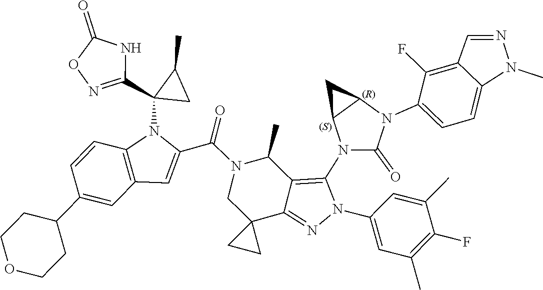 | 3-((1S,2S)-1-(2-((S)-3′- ((1S,5R)-4-(4-fluoro-1- methyl-1H-indazol-5-yl)-3- oxo-2,4- diazabicyclo[3.1.0]hexan-2- yl)-2′-(4-fluoro-3,5- dimethylphenyl)-4′-methyl- 2′,4′,5′,6′- tetrahydrospiro[cyclopropane- 1,7′-pyrazolo[4,3-c]pyridine]- 5′-carbonyl)-5-(tetrahydro-2H- pyran-4-yl)-1H-indol-1-yl)-2- methylcyclopropyl)-1,2,4- oxadiazol-5(4H)-one or 3- ((1S,2S)-1-(2-((S)-3′-((1R,5S)- 4-(4-fluoro-1-methyl-1H- indazol-5-yl)-3-oxo-2,4- diazabicyclo[3.1.0]hexan-2- yl)-2′-(4-fluoro-3,5- | 895.6 |
| | | dimethylphenyl)-4′-methyl- | |
| | | 2′,4′,5′,6′- | |
| | | tetrahydrospiro[cyclopropane- | |
| | | 1,7′-pyrazolo[4,3-c]pyridine]- | |
| | | 5′-carbonyl)-5-(tetrahydro-2H- | |
| | | pyran-4-yl)-1H-indol-1-yl)-2- | |
| | | methylcyclopropyl)-1,2,4- | |
| | | oxadiazol-5(4H)-one | |
| | or | | |
| | 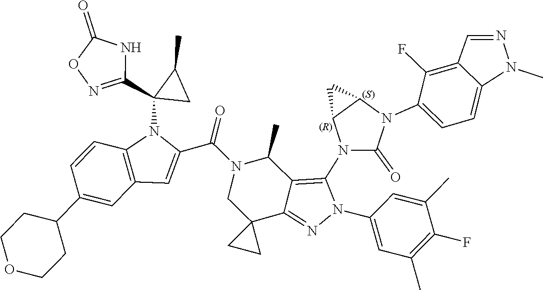 | | |
| 117 Isomer B | 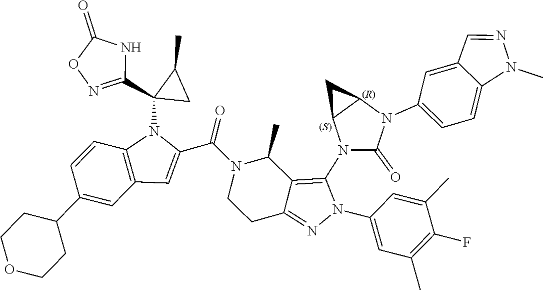 | 3-((1S,2S)-1-(2-((S)-2-(4- fluoro-3,5-dimethylphenyl)-4- methyl-3-((1S,5R)-4-(1- methyl-1H-indazol-5-yl)-3- oxo-2,4- diazabicyclo[3.1.0]hexan-2- yl)-4,5,6,7-tetrahydro-2H- pyrazolo[4,3-c]pyridine-5- carbonyl)-5-(tetrahydro-2H- pyran-4-yl)-1H-indol-1-yl)-2- methylcyclopropyl)-1,2,4- oxadiazol-5(4H)-one or 3- ((1S,2S)-1-(2-((S)-2-(4-fluoro- 3,5-dimethylphenyl)-4-methyl- 3-((1R,5S)-4-(1-methyl-1H- indazol-5-yl)-3-oxo-2,4- diazabicyclo[3.1.0]hexan-2- yl)-4,5,6,7-tetrahydro-2H- | 851.7 |
| | | pyrazolo[4,3-c]pyridine-5- | |
| | | carbonyl)-5-(tetrahydro-2H- | |
| | | pyran-4-yl)-1H-indol-1-yl)-2- | |
| | | methylcyclopropyl)-1,2,4- | |
| | | oxadiazol-5(4H)-one | |
| | or | | |
| | 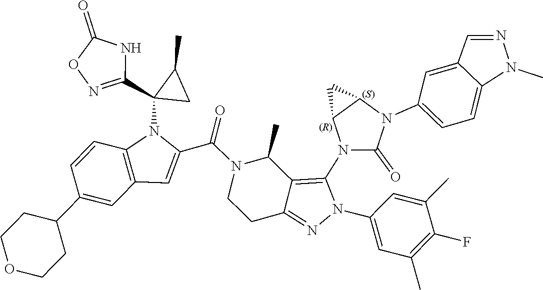 | | |
| 118 Isomer B | 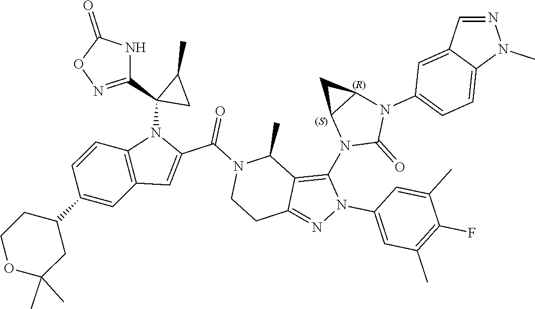 | 3-((1S,2S)-1-(5-((S)-2,2- dimethyltetrahydro-2H-pyran- 4-yl)-2-((S)-2-(4-fluoro-3,5- dimethylphenyl)-4-methyl-3- ((1S,5R)-4-(1-methyl-1H- indazol-5-yl)-3-oxo-2,4- diazabicyclo[3.1.0]hexan-2- yl)-4,5,6,7-tetrahydro-2H- pyrazolo[4,3-c]pyridine-5- carbonyl)-1H-indol-1-yl)-2- methylcyclopropyl)-1,2,4- oxadiazol-5(4H)-one or 3- ((1S,2S)-1-(5-((S)-2,2- dimethyltetrahydro-2H-pyran- 4-yl)-2-((S)-2-(4-fluoro-3,5- dimethylphenyl)-4-methyl-3- ((1R,5S)-4-(1-methyl-1H- indazol-5-yl)-3-oxo-2,4- diazabicyclo[3.1.0]hexan-2- yl)-4,5,6,7-tetrahydro-2H- | 879.7 |
| | | pyrazolo[4,3-c]pyridine-5- | |
| | | carbonyl)-1H-indol-1-yl)-2- | |
| | | methylcyclopropyl)-1,2,4- | |
| | | oxadiazol-5(4H)-one | |
| | or | | |
| | 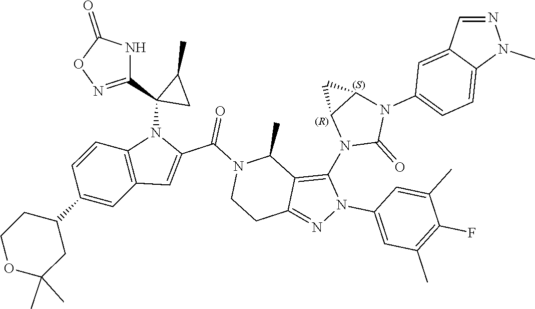 | | |
| 119 Isomer B | 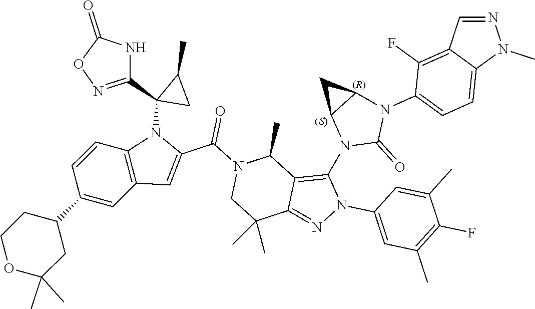 | 3-((1S,2S)-1-(5-((S)-2,2- dimethyltetrahydro-2H-pyran- 4-yl)-2-((S)-3-((1S,5R)-4-(4- fluoro-1-methyl-1H-indazol-5- yl)-3-oxo-2,4- diazabicyclo[3.1.0]hexan-2- yl)-2-(4-fluoro-3,5- dimethylphenyl)-4,7,7- trimethyl-4,5,6,7-tetrahydro- 2H-pyrazolo[4,3-c]pyridine-5- carbonyl)-1H-indol-1-yl)-2- methylcyclopropyl)-1,2,4- oxadiazol-5(4H)-one or 3- ((1S,2S)-1-(5-((S)-2,2- dimethyltetrahydro-2H-pyran- 4-yl)-2-((S)-3-((1R,5S)-4-(4- fluoro-1-methyl-1H-indazol-5- yl)-3-oxo-2,4- diazabicyclo[3.1.0]hexan-2- yl)-2-(4-fluoro-3,5- | 925.8 |
| | | dimethylphenyl)-4,7,7- | |
| | | trimethyl-4,5,6,7-tetrahydro- | |
| | | 2H-pyrazolo[4,3-c]pyridine-5- | |
| | | carbonyl)-1H-indol-1-yl)-2- | |
| | | methylcyclopropyl)-1,2,4- | |
| | | oxadiazol-5(4H)-one | |
| | or | | |
| | 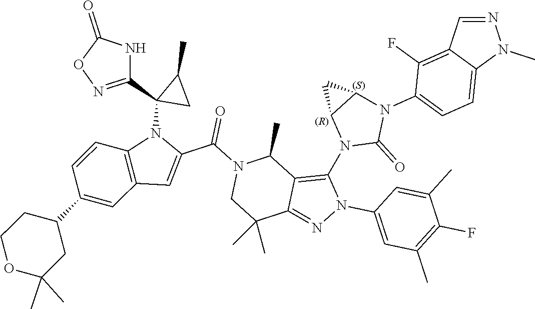 | | |
| 120 Isomer B | 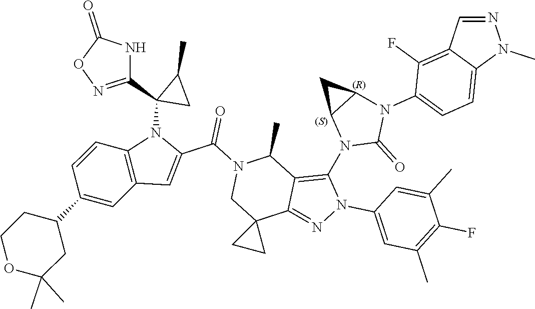 | 3-((1S,2S)-1-(5-((S)-2,2- dimethyltetrahydro-2H-pyran- 4-yl)-2-((S)-3′-((1S,5R)-4-(4- fluoro-1-methyl-1H-indazol-5- yl)-3-oxo-2,4- diazabicyclo[3.1.0]hexan-2- yl)-2′-(4-fluoro-3,5- dimethylphenyl)-4′-methyl- 2′,4′,5′,6′- tetrahydrospiro[cyclopropane- 1,7′-pyrazolo[4,3-c]pyridine]- 5′-carbonyl)-1H-indol-1-yl)-2- methylcyclopropyl)-1,2,4- oxadiazol-5(4H)-one or 3- ((1S,2S)-1-(5-((S)-2,2- dimethyltetrahydro-2H-pyran- 4-yl)-2-((S)-3′-((1R,5S)-4-(4- fluoro-1-methyl-1H-indazol-5- yl)-3-oxo-2,4- | 923.8 |
| | | diazabicyclo[3.1.0]hexan-2- | |
| | | yl)-2′-(4-fluoro-3,5- | |
| | | dimethylphenyl)-4′-methyl- | |
| | | 2′,4′,5′,6′- | |
| | | tetrahydrospiro[cyclopropane- | |
| | | 1,7′-pyrazolo[4,3-c]pyridine]- | |
| | | 5′-carbonyl)-1H-indol-1-yl)-2- | |
| | | methylcyclopropyl)-1,2,4- | |
| | | oxadiazol-5(4H)-one | |
| | or | | |
| | 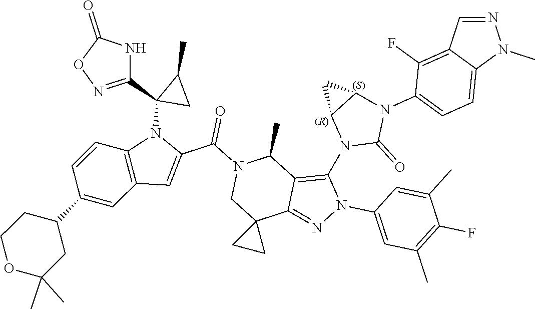 | | |
| 121 Isomer B | 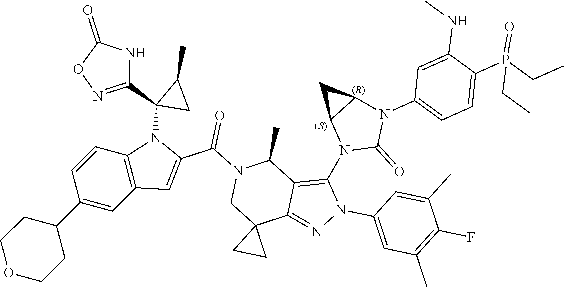 | 3-((1S,2S)-1-(2-((S)-3′- ((1S,5R)-4-(4- (diethylphosphoryl)-3- (methylamino)phenyl)-3-oxo- 2,4-diazabicyclo[3.1.0]hexan- 2-yl)-2′-(4-fluoro-3,5- dimethylphenyl)-4′-methyl- 2′,4′,5′,6′- tetrahydrospiro[cyclopropane- 1,7′-pyrazolo[4,3-c]pyridine]- 5′-carbonyl)-5-(tetrahydro-2H- pyran-4-yl)-1H-indol-1-yl)-2- methylcyclopropyl)-1,2,4- oxadiazol-5(4H)-one or 3- ((1S,2S)-1-(2-((S)-3′-((1R,5S)- 4-(4-(diethylphosphoryl)-3- (methylamino)phenyl)-3-oxo- 2,4-diazabicyclo[3.1.0]hexan- 2-yl)-2′-(4-fluoro-3,5- dimethylphenyl)-4′-methyl- | 956.8 |
| | | 2′,4′,5′,6′- | |
| | | tetrahydrospiro[cyclopropane- | |
| | | 1,7′-pyrazolo[4,3-c]pyridine]- | |
| | | 5′-carbonyl)-5-(tetrahydro-2H- | |
| | | pyran-4-yl)-1H-indol-1-yl)-2- | |
| | | methylcyclopropyl)-1,2,4- | |
| | | oxadiazol-5(4H)-one | |
| | or | | |
| | 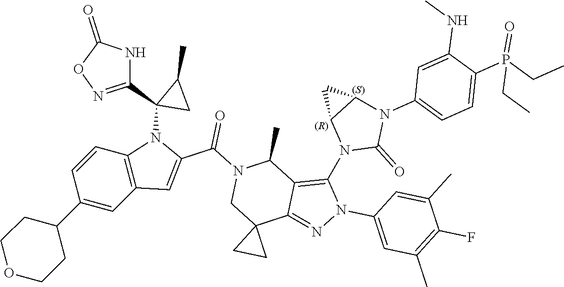 | | |
| 122 Isomer B | 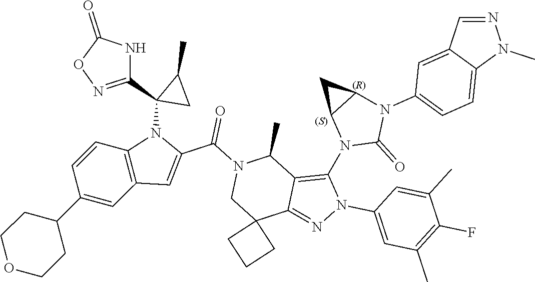 | 3-((1S,2S)-1-(2-((S)-3′- ((1R,5S)-4-(4-fluoro-1- methyl-1H-indazol-5-yl)-3- oxo-2,4- diazabicyclo[3.1.0]hexan-2- yl)-2′-(4-fluoro-3,5- dimethylphenyl)-4′-methyl- 2′,4′,5′,6′- tetrahydrospiro[cyclobutane- 1,7′-pyrazolo[4,3-c]pyridine]- 5′-carbonyl)-5-(tetrahydro-2H- pyran-4-yl)-1H-indol-1-yl)-2- methylcyclopropyl)-1,2,4- oxadiazol-5(4H)-one or 3- ((1S,2S)-1-(2-((S)-3′-((1S,5R)- 4-(4-fluoro-1-methyl-1H- indazol-5-yl)-3-oxo-2,4- diazabicyclo[3.1.0]hexan-2- | 909.4 |
| | | yl)-2′-(4-fluoro-3,5- | |
| | | dimethylphenyl)-4′-methyl- | |
| | | 2′,4′,5′,6′- | |
| | | tetrahydrospiro[cyclobutane- | |
| | | 1,7′-pyrazolo[4,3-c]pyridine]- | |
| | | 5′-carbonyl)-5-(tetrahydro-2H- | |
| | | pyran-4-yl)-1H-indol-1-yl)-2- | |
| | | methylcyclopropyl)-1,2,4- | |
| | | oxadiazol-5(4H)-one | |
| | or | | |
| | 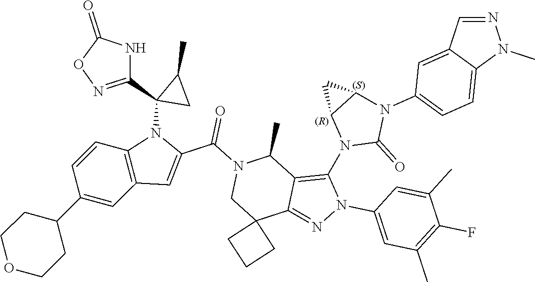 | | |
| 123 Isomer B | 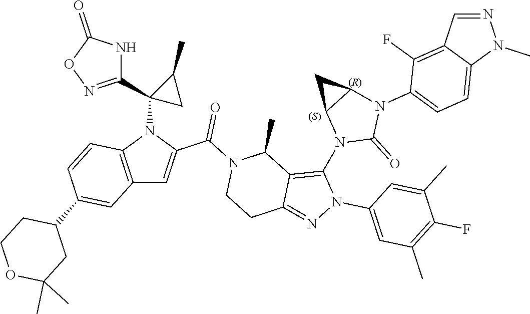 | 3-((1S,2S)-1-(5-((S)-2,2- dimethyltetrahydro-2H-pyran- 4-yl)-2-((S)-2-(4-fluoro-3,5- dimethylphenyl)-4-methyl-3- ((1S,5R)-4-(1-methyl-1H- indazol-5-yl)-3-oxo-2,4- diazabicyclo[3.1.0]hexan-2- yl)-4,5,6,7-tetrahydro-2H- pyrazolo[4,3-c]pyridine-5- carbonyl)-1H-indol-1-yl)-2- methylcyclopropyl)-1,2,4- oxadiazol-5(4H)-one or 3- ((1S,2S)-1-(5-((S)-2,2- dimethyltetrahydro-2H-pyran- 4-yl)-2-((S)-2-(4-fluoro-3,5- dimethylphenyl)-4-methyl-3- ((1R,5S)-4-(1-methyl-1H- indazol-5-yl)-3-oxo-2,4- diazabicyclo[3.1.0]hexan-2- yl)-4,5,6,7-tetrahydro-2H- | 897.6 |
| | | pyrazolo[4,3-c]pyridine-5- | |
| | | carbonyl)-1H-indol-1-yl)-2- | |
| | | methylcyclopropyl)-1,2,4- | |
| | | oxadiazol-5(4H)-one | |
| | or | | |
| | 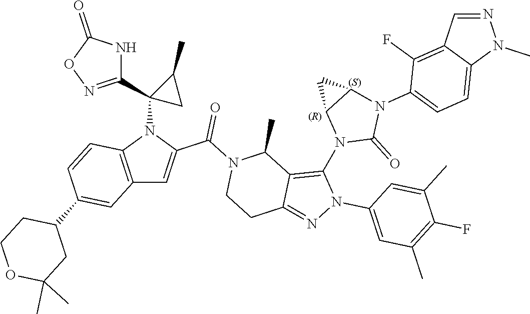 | | |
| 215 | 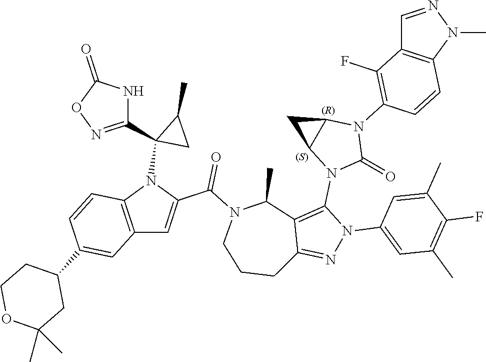 | 3-[(1S,2S)-1-[5-[(4S)-2,2- dimethyltetrahydropyran-4- yl]-2-[(4S)-2-(4-fluoro-3,5- dimethyl-phenyl)-3-[4-(4- fluoro-1-methyl-indazol-5-yl)- 3-oxo-2,4- diazabicyclo[3.1.0]hexan-2- yl]-4-methyl-4,6,7,8- tetrahydropyrazolo [4,3- c]azepin-1-ium-5- carbonyl]indol-1-yl]-2-methyl- cyclopropyl]-4H-1,2,4- oxadiazol-5-one formate | 911.4 |
| | or | | |
| | 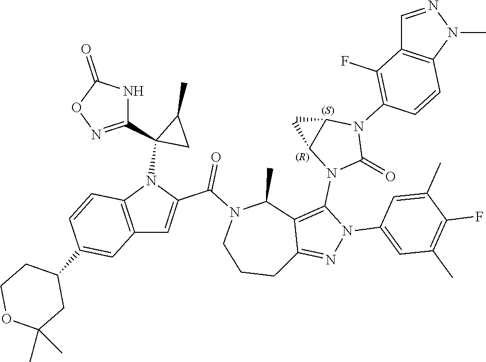 | | |

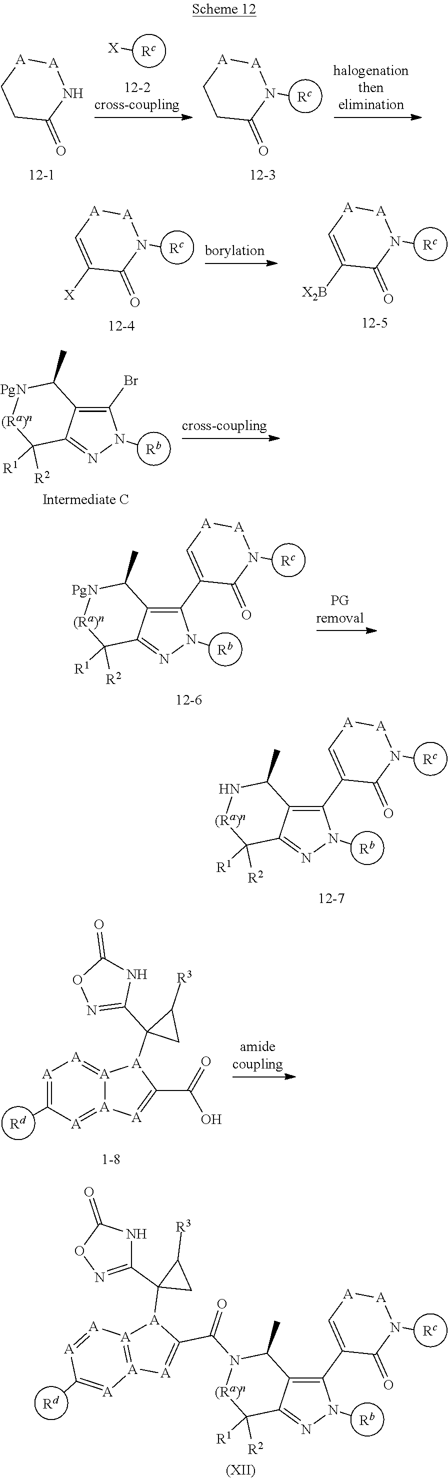

Compounds of formula (XII) are prepared from intermediate 12-1 by coupling with alkyl, aryl or heteroaryl halides or boronic acid or other boronate partners (12-2) via transition metal catalysis (e.g., Cu—or Pd-catalysis) or basic conditions. Subsequently, amide 12-3 is halogenated (e.g., treatment with PCl₅) then dehydrohalogenated (e.g., basic conditions) to provide 12-4. Alkenyl halide 12-4 is sequentially converted to boronate 12-5 that is then coupled with intermediate C to provide 12-6 via transition metal catalyzed cross-coupling conditions (e.g., Pd-catalysis). Removal of the protecting group (e.g., acidic conditions) of 12-6 provides amine 12-7 that is then coupled with carboxylic acid 1-8 to provide compounds of formula (XII).

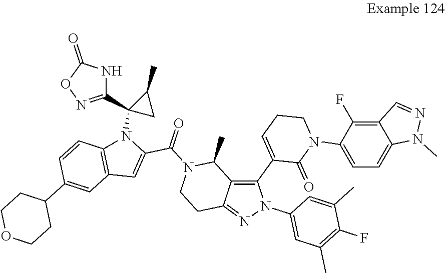 [SMILES: Cc1cc(-n2nc3c(c2C2=CCCN(c4ccc5c(cnn5C)c4F)C2=O)[C@H](C)N(C(=O)c2cc4cc(C5CCOCC5)ccc4n2[C@@]2(c4noc(=O)[nH]4)C[C@@H]2C)CC3)cc(C)c1F]

### 3-((1S,2S)-1-(2-((S)-3-(1-(4-fluoro-1-methyl-1H-indazol-5-yl)-2-oxo-1,2,5,6-tetrahydropyridin-3-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one (scheme 12)

### Step 1: 1-(4-fluoro-1-methyl-1H-indazol-5-yl)piperidin-2-one

A mixture of 5-bromo-4-fluoro-1-methyl-1H-indazole (2.2 g, 9.6 mmol), piperidin-2-one (1.4 g, 14 mmol), CuI (0.55 g, 2.9 mmol), K₂CO₃ (4.0 g, 29 mmol) and N1,N2-dimethylethane-1,2-diamine (0.25 g, 2.9 mmol) in DMF (45 mL) was stirred at 135° C. for 32 h and then cooled to RT. The mixture was filtered through Celite® and washed with DCM (3×50 mL). The filtrate was concentrated under reduced pressure. EtOAc (100 mL) and water (35 mL) were added and the layers were separated. The organic layer was washed with brine (2×30 mL), dried over Na₂SO₄, filtered, and concentrated under reduced pressure. The material was purified by reverse phase chromatography (5 to 100% MeCN/water, contains 0.1% formic acid) to afford the title compound. MS (ESI) m/z 248.2 [M+H]+

### Step 2: 3-chloro-1-(4-fluoro-1-methyl-1H-indazol-5-yl)-5,6-dihydropyridin-2(1H)-one

To a solution of 1-(4-fluoro-1-methyl-1H-indazol-5-yl)piperidin-2-one (1.0 g, 4.0 mmol) in chloroform (20 mL) was added PCl₅ (2.5 g, 12 mmol) slowly (via drop-wise addition over 15 min). The reaction mixture was stirred at 60° C. for 3 h. Then, the mixture was cooled to rt and slowly poured onto ice water (30 mL) with vigorous stirring and maintaining the temperature of the resulting mixture below 20° C. After stirring an additional 10 min, the mixture was extracted with DCM and the extracts were washed with aqueous sodium bicarbonate then brine, dried over sodium sulfate, and concentrated under reduced pressure to afford 3,3-dichloro-1-(4-fluoro-1-methyl-1H-indazol-5-yl)piperidin-2-one. MS (ESI) m/z 316.0, 318.0 [M+H]+

3,3-Dichloro-1-(4-fluoro-1-methyl-1H-indazol-5-yl)piperidin-2-one (2.8 g, 8.9 mmol) from the previous reaction was dissolved in DMF (35 mL). Lithium chloride (0.38 g, 8.9 mmol) and lithium carbonate (1.3 g, 18 mmol) were added, and the resulting mixture was stirred at 130° C. for 4 h under an atmosphere of nitrogen. After cooling to rt, the mixture was quenched by addition of H₂O and stirred for 5 min. The aqueous phase was extracted with EtOAc (2 x). The combined organic layers were washed with sat a NaHCO₃ (1 x) and brine (2 x), dried over sodium sulfate and concentrated under reduced pressure to afford the title compound. MS (ESI) m/z 280.1, 282.1 [M+H]+

### Step 3: 1-(4-fluoro-1-methyl-1H-indazol-5-yl)-3-(4,4,5,5-tetramethyl-1,3,2-dioxaborolan-2-yl)-5,6-dihydropyridin-2(1H)-one

To a solution of the 3-chloro-1-(4-fluoro-1-methyl-1H-indazol-5-yl)-5,6-dihydropyridin-2(1H)-one (151 mg, 0.540 mmol), bis(pinacolato)diborane (178 mg, 0.702 mmol) in toluene (5 mL) was added potassium acetate (159 mg, 1.62 mmol). Argon was bubbled through the mixture for 10 min, then BrettPhos Pd G3 (97.9 mg, 0.108 mmol) was added, and argon was again bubbled through the mixture for 5 min. The reaction mixture was stirred at 105° C. for 2 h, then cooled to rt to afford the title compound as a mixture for the next step. MS (ESI) m/z 372.3 [M+H]+

### Step 4: tert-butyl (S)-3-(1-(4-fluoro-1-methyl-1H-indazol-5-yl)-2-oxo-1,2,5,6-tetrahydropyridin-3-yl)-2-(4-fluoro-3,5-dimethylphenyl)-4-methyl-2,4,6,7-tetrahydro-5H-pyrazolo[4,3-c]pyridine-5-carboxylate

To a solution of 1-(4-fluoro-1-methyl-1H-indazol-5-yl)-3-(4,4,5,5-tetramethyl-1,3,2-dioxaborolan-2-yl)-5,6-dihydropyridin-2(1H)-one (190 mg, 0.512 mmol), tert-butyl (S)-3-bromo-2-(4-fluoro-3,5-dimethylphenyl)-4-methyl-2,4,6,7-tetrahydro-5H-pyrazolo[4,3-c]pyridine-5-carboxylate (179 mg, 0.409 mmol) in toluene (5.0 mL) was added the potassium phosphate, tribasic (326 mg, 1.54 mmol). Argon was bubbled through the mixture for 10 min, then BrettPhos Pd G3 (92.8 mg, 0.102 mmol) was added, and argon was again bubbled through the mixture for 5 min. The reaction mixture was stirred at 105° C. for 1 hour, then cooled to rt. The mixture was filtered through Celite® and washed with DCM (3×15 mL). The filtrate was concentrated under reduced pressure. The crude material was purified by reverse-phase chromatography (5 to 100% MeCN/water, contains 0.1% formic acid) to afford the title compound. MS (ESI) m/z 603.4 [M+H]+

### Step 5: (S)-1-(4-fluoro-1-methyl-1H-indazol-5-yl)-3-(2-(4-fluoro-3,5-dimethylphenyl)-4-methyl-4,5,6,7-tetrahydro-2H-pyrazolo[4,3-c]pyridin-3-yl)-5,6-dihydropyridin-2(1H)-one

A mixture of tert-butyl (S)-3-(1-(4-fluoro-1-methyl-1H-indazol-5-yl)-2-oxo-1,2,5,6-tetrahydropyridin-3-yl)-2-(4-fluoro-3,5-dimethylphenyl)-4-methyl-2,4,6,7-tetrahydro-5H-pyrazolo[4,3-c]pyridine-5-carboxylate (40 mg, 66 mol) in HCl (0.17 mL, 4 molar, 0.66 mmol) in dioxane under an atmosphere of nitrogen at rt was stirred at 23° C. for 1 hour, then Et₂O was added. The resulting precipitate was filtered and washed with Et₂O (2×7 mL) to afford the title compound. MS (ESI) m/z 503.3 [M+H]+

### Step 6: 3-((1S,2S)-1-(2-((S)-3-(1-(4-fluoro-1-methyl-1H-indazol-5-yl)-2-oxo-1,2,5,6-tetrahydropyridin-3-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one

To a solution of (S)-1-(4-fluoro-1-methyl-1H-indazol-5-yl)-3-(2-(4-fluoro-3,5-dimethylphenyl)-4-methyl-4,5,6,7-tetrahydro-2H-pyrazolo[4,3-c]pyridin-3-yl)-5,6-dihydropyridin-2(1H)-one hydrochloride salt (29.5 mg, 54.7 mol), 1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carboxylic acid (23.1 mg, 60.2 mol), and HATU (25.0 mg, 65.7 mol) in DMF (0.9 mL) was added DIPEA (21.2 mg, 164 mol). The mixture was stirred at 23° C. for 1 hour. Then, the reaction mixture was directly purified by reverse-phase HPLC (5 to 100% MeCN/water, contains 0.1% formic acid) to provide the title compound. ¹H NMR (400 MHz, methanol-d4): δH 7.85-8.08 (1H, m), 7.49-7.56 (2H, m), 7.41-7.43 (1H, m), 7.17-7.28 (3H, m), 7.05-7.08 (2H, m), 6.86 (1H, s), 5.30-5.80 (0.5H, m), 4.43-4.76 (0.5H, m), 4.04-4.07 (6H, m), 3.52-3.92 (4H, m), 2.54-3.17 (4H, m), 2.22-2.32 (7H, m), 1.71-1.81 (6H, m), 1.66-1.68 (1H, m), 1.52-1.55 (3H, m), 1.36-1.43 (1H, m), 1.21-1.24 (2H, m), 0.99-1.01 (1H, m). MS (ESI) m/z 868.5 [M+H]+.

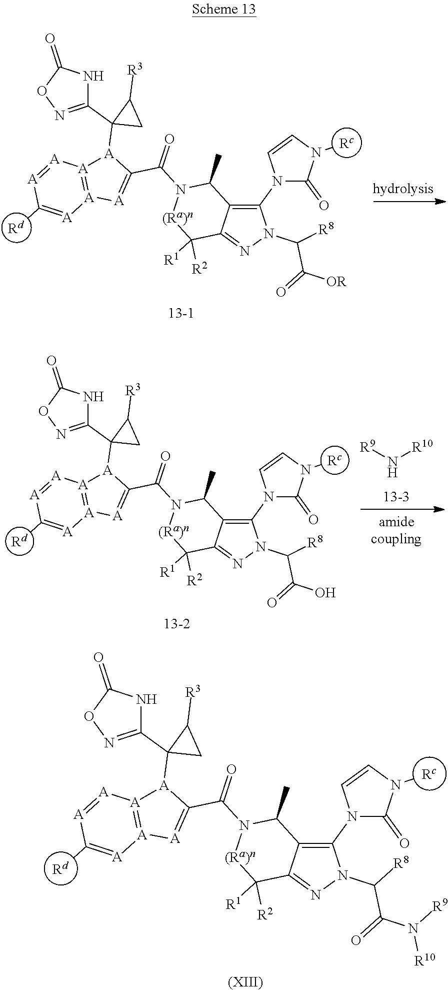

Compounds of formula (XIII) are prepared from intermediate 13-1 by hydrolysis of the ester (e.g., basic aqueous conditions). Subsequently, acid 13-2 is coupled with amines 13-3 to provide compounds of formula (XIII).

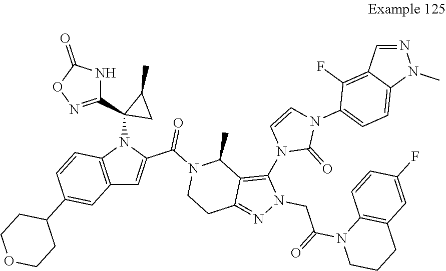 [SMILES: C[C@H]1c2c(nn(CC(=O)N3CCCc4cc(F)ccc43)c2-n2ccn(-c3ccc4c(cnn4C)c3F)c2=O)CCN1C(=O)c1cc2cc(C3CCOCC3)ccc2n1[C@@]1(c2noc(=O)[nH]2)C[C@@H]1C]

3-((1S,2S)-1-(2-((S)-3-(3-(4-fluoro-1-methyl-1H-indazol-5-yl)-2-oxo-2,3-dihydro-1H-imidazol-1-yl)-2-(2-(6-fluoro-3,4-dihydroquinolin-1(2H)-yl)-2-oxoethyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one (scheme 13)

### Step 1: 2-((S)-3-(3-(4-fluoro-1-methyl-1H-indazol-5-yl)-2-oxo-2,3-dihydro-1H-imidazol-1-yl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-2-yl)acetic acid

To a mixture of ethyl 2-((S)-3-(3-(4-fluoro-1-methyl-1H-indazol-5-yl)-2-oxo-2,3-dihydro-1H-imidazol-1-yl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-2-yl)acetate (900 mg, 1.099 mmol) in ethanol (12 mL), water (4 mL) and THF (4 mL) was added LiOH H₂O (138 mg, 3.30 mmol) in a glove box. The mixture was stirred at 20° C. for 1 h. The mixture was adjusted pH to 4-5 with HCl (2M). Then the crude residue was purified by reverse-phase HPLC (45-65% MeCN/water+0.1% TFA) to provide the title compound. LCMS (ESI) m/z: 791.3 [M+H]⁺. ¹H NMR (400 MHz, DMSO-d6) δ=11.86-11.68 (m, 1H), 8.31 (s, 1H), 7.68-7.63 (m, 1H), 7.61-7.50 (m, 2H), 7.44-7.37 (m, 1H), 7.31-7.24 (m, 1H), 7.16-7.10 (m, 1H), 7.00-6.63 (m, 3H), 5.65-5.51 (m, 1H), 4.97-4.88 (m, 2H), 4.41-4.28 (m, 1H), 4.16-4.04 (m, 4H), 3.97 (br d, J=10.7 Hz, 3H), 3.16-3.02 (m, 1H), 2.93-2.72 (m, 3H), 1.77-1.64 (m, 7H), 1.37-1.30 (m, 3H), 1.23-1.12 (m, 3H)

### Step 2: 3-((1S,2S)-1-(2-((S)-3-(3-(4-fluoro-1-methyl-1H-indazol-5-yl)-2-oxo-2,3-dihydro-1H-imidazol-1-yl)-2-(2-(6-fluoro-3,4-dihydroquinolin-1(2H)-yl)-2-oxoethyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one

To a vial containing 6-fluoro-1,2,3,4-tetrahydroquinoline (3.4 mg, 0.023 mmol) was added stock solutions of 2-((S)-3-(3-(4-fluoro-1-methyl-1H-indazol-5-yl)-2-oxo-2,3-dihydro-1H-imidazol-1-yl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-2-yl)acetic acid (6 mg, 0.008 mmol) in DMF (0.05 mL), HATU (4.3 mg, 0.011 mmol) in DMF (0.03 mL) and DIPEA (6.6 mL, 0.038 mmol) in DMF (0.05 mL). The resulting mixture was stirred at room temperature for 15 h. The reaction solution was filtered, and an additional 250 μL of DMF was used to wash the filter plate. The reaction was purified by reverse-phase hplc 30 to 70% MeCN/water+0.1% formic acid) to provide the title compound. LCMS (ESI) m/z: 924.9 [M+H]⁺. ¹H NMR (500 MHz, DMSO) δ 8.23-8.32 (m, 1H), 7.58-7.68 (m, 2H), 7.34-7.42 (m, 2H), 7.04-7.18 (m, 3H), 6.86-6.92 (s, 1H), 6.41-6.60 (m, 1H), 5.6 (s, 1H), 5.08-5.28 (m, 1H), 4.12 (s, 3H), 3.97 (d, J=9.9 Hz, 2H), 3.31-3.37 (m, 3H), 2.77-2.88 (m, 1H), 2.66-2.76 (m, 1H), 1.80-1.93 (m, 2H), 1.65-1.80 (m, 5H), 1.40-1.48 (m, 1H), 1.10-1.30 (m, 7H). The following examples in table 11 were prepared according to scheme 13 using the procedures outlined in the synthesis of examples 125.

TABLE 11

| | | | MS |
|---|---|---|---|
| Ex. | Structure | Name | (M + 1) |
| 126 | 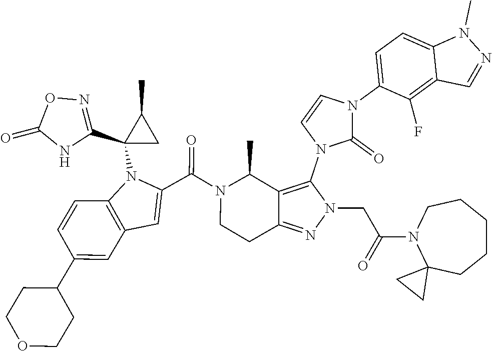 [SMILES: C[C@H]1c2c(nn(CC(=O)N3CCCCCC34CC4)c2-n2ccn(-c3ccc4c(cnn4C)c3F)c2=O)CCN1C(=O)c1cc2cc(C3CCOCC3)ccc2n1[C@@]1(c2noc(=O)[nH]2)C[C@@H]1C] | 3-((1S,2S)-1-(2-((S)-3-(3- (4-fluoro-1-methyl-1H- indazol-5-yl)-2-oxo-2,3- dihydro-1H-imidazol-1- yl)-4-methyl-2-(2-oxo-2- (4-azaspiro[2.6]nonan-4- yl)ethyl)-4,5,6,7- tetrahydro-2H- pyrazolo[4,3-c]pyridine- 5-carbonyl)-5- (tetrahydro-2H-pyran-4- yl)-1H-indol-1-yl)-2- methylcyclopropyl)- 1,2,4-oxadiazol-5(4H)- one | 898.8 |
| 127 | 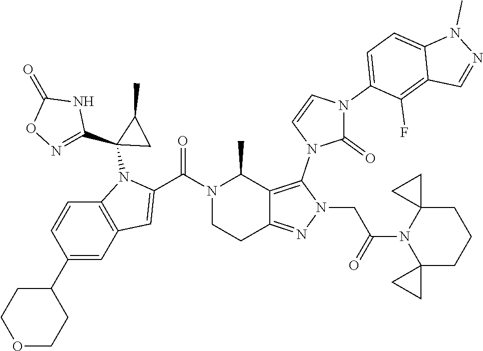 [SMILES: C[C@H]1c2c(nn(CC(=O)N3C4(CCCC35CC5)CC4)c2-n2ccn(-c3ccc4c(cnn4C)c3F)c2=O)CCN1C(=O)c1cc2cc(C3CCOCC3)ccc2n1[C@@]1(c2noc(=O)[nH]2)C[C@@H]1C] | 3-((1S,2S)-1-(2-((S)-2-(2- (4- azadispiro[2.1.25.33]decan- 4-yl)-2-oxoethyl)-3-(3- (4-fluoro-1-methyl-1H- indazol-5-yl)-2-oxo-2,3- dihydro-1H-imidazol-1- yl)-4-methyl-4,5,6,7- tetrahydro-2H- pyrazolo[4,3-c]pyridine- 5-carbonyl)-5- (tetrahydro-2H-pyran-4- yl)-1H-indol-1-yl)-2- methylcyclopropyl)- 1,2,4-oxadiazol-5(4H)- one | 910.8 |
| 128 | 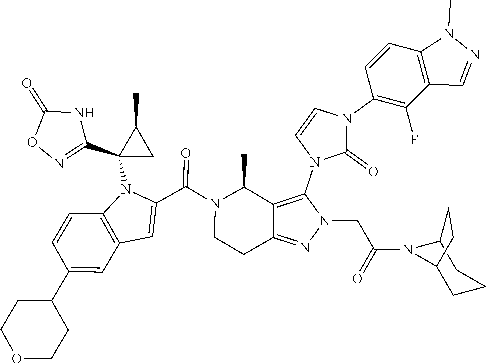 [SMILES: C[C@H]1c2c(nn(CC(=O)N3C4CCCC3CC4)c2-n2ccn(-c3ccc4c(cnn4C)c3F)c2=O)CCN1C(=O)c1cc2cc(C3CCOCC3)ccc2n1[C@@]1(c2noc(=O)[nH]2)C[C@@H]1C] | 3-((1S,2S)-1-(2-((S)-2-(2- ((1R,5S)-8- azabicyclo[3.2.1]octan-8- yl)-2-oxoethyl)-3-(3-(4- fluoro-1-methyl-1H- indazol-5-yl)-2-oxo-2,3- dihydro-1H-imidazol-1- yl)-4-methyl-4,5,6,7- tetrahydro-2H- pyrazolo[4,3-c]pyridine- 5-carbonyl)-5- (tetrahydro-2H-pyran-4- yl)-1H-indol-1-yl)-2- methylcyclopropyl)- 1,2,4-oxadiazol-5(4H)- one | 884.9 |
| 129 | 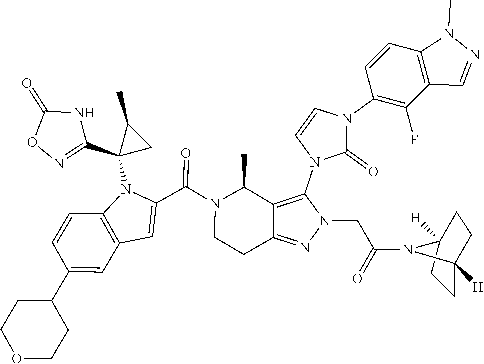 [SMILES: C[C@H]1c2c(nn(CC(=O)N3[C@H]4CC[C@@H]3CC4)c2-n2ccn(-c3ccc4c(cnn4C)c3F)c2=O)CCN1C(=O)c1cc2cc(C3CCOCC3)ccc2n1[C@@]1(c2noc(=O)[nH]2)C[C@@H]1C] | 3-((1S,2S)-1-(2-((S)-2-(2- ((1R,4R)-7- azabicyclo[2.2.1]heptan- 7-yl)-2-oxoethyl)-3-(3-(4- fluoro-1-methyl-1H- indazol-5-yl)-2-oxo-2,3- dihydro-1H-imidazol-1- yl)-4-methyl-4,5,6,7- tetrahydro-2H- pyrazolo[4,3-c]pyridine- 5-carbonyl)-5- (tetrahydro-2H-pyran-4- yl)-1H-indol-1-yl)-2- methylcyclopropyl)- 1,2,4-oxadiazol-5(4H)- one | 870.7 |
| 130 | 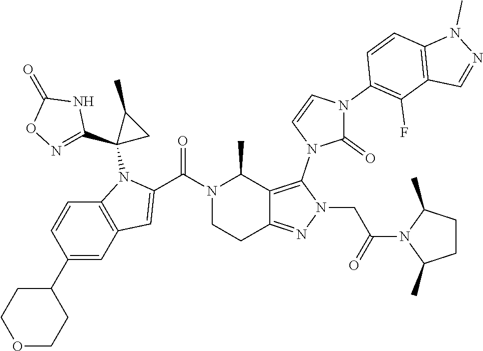 [SMILES: C[C@@H]1CC[C@H](C)N1C(=O)Cn1nc2c(c1-n1ccn(-c3ccc4c(cnn4C)c3F)c1=O)[C@H](C)N(C(=O)c1cc3cc(C4CCOCC4)ccc3n1[C@@]1(c3noc(=O)[nH]3)C[C@@H]1C)CC2] | 3-((1S,2S)-1-(2-((S)-2-(2- ((2S,5R)-2,5- dimethylpyrrolidin-1-yl)- 2-oxoethyl)-3-(3-(4- fluoro-1-methyl-1H- indazol-5-yl)-2-oxo-2,3- dihydro-1H-imidazol-1- yl)-4-methyl-4,5,6,7- tetrahydro-2H- pyrazolo[4,3-c]pyridine- 5-carbonyl)-5- (tetrahydro-2H-pyran-4- yl)-1H-indol-1-yl)-2- methylcyclopropyl)- 1,2,4-oxadiazol-5(4H)- one | 872.4 |
| 131 | 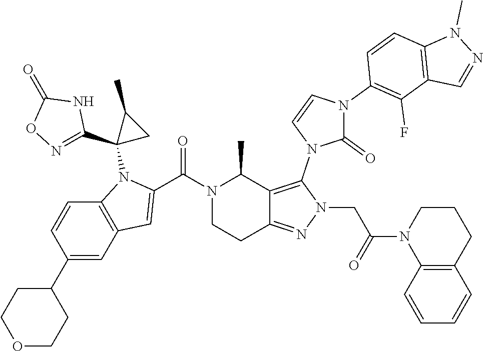 [SMILES: C[C@H]1c2c(nn(CC(=O)N3CCCc4ccccc43)c2-n2ccn(-c3ccc4c(cnn4C)c3F)c2=O)CCN1C(=O)c1cc2cc(C3CCOCC3)ccc2n1[C@@]1(c2noc(=O)[nH]2)C[C@@H]1C] | 3-((1S,2S)-1-(2-((S)-2-(2- (3,4-dihydroquinolin- 1(2H)-yl)-2-oxoethyl)-3- (3-(4-fluoro-1-methyl- 1H-indazol-5-yl)-2-oxo- 2,3-dihydro-1H-imidazol- 1-yl)-4-methyl-4,5,6,7- tetrahydro-2H- pyrazolo[4,3-c]pyridine- 5-carbonyl)-5- (tetrahydro-2H-pyran-4- yl)-1H-indol-1-yl)-2- methylcyclopropyl)- 1,2,4-oxadiazol-5(4H)- one | 906.4 |
| 132 | 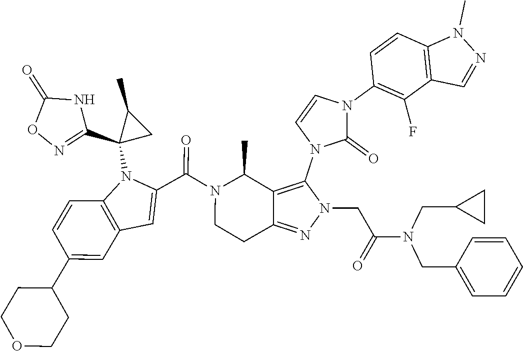 [SMILES: C[C@H]1c2c(nn(CC(=O)N(Cc3ccccc3)CC3CC3)c2-n2ccn(-c3ccc4c(cnn4C)c3F)c2=O)CCN1C(=O)c1cc2cc(C3CCOCC3)ccc2n1[C@@]1(c2noc(=O)[nH]2)C[C@@H]1C] | N-benzyl-N- (cyclopropylmethyl)-2- ((S)-3-(3-(4-fluoro-1- methyl-1H-indazol-5-yl)- 2-oxo-2,3-dihydro-1H- imidazol-1-yl)-4-methyl- 5-(1-((1S,2S)-2-methyl-1- (5-oxo-4,5-dihydro-1,2,4- oxadiazol-3- yl)cyclopropyl)-5- (tetrahydro-2H-pyran-4- yl)-1H-indole-2- carbonyl)-4,5,6,7- tetrahydro-2H- pyrazolo[4,3-c]pyridin-2- yl)acetamide | 934.4 |
| 133 | 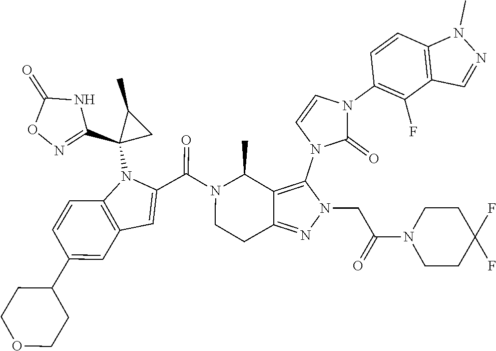 [SMILES: C[C@H]1c2c(nn(CC(=O)N3CCC(F)(F)CC3)c2-n2ccn(-c3ccc4c(cnn4C)c3F)c2=O)CCN1C(=O)c1cc2cc(C3CCOCC3)ccc2n1[C@@]1(c2noc(=O)[nH]2)C[C@@H]1C] | 3-((1S,2S)-1-(2-((S)-2-(2- (4,4-difluoropiperidin-1- yl)-2-oxoethyl)-3-(3-(4- fluoro-1-methyl-1H- indazol-5-yl)-2-oxo-2,3- dihydro-1H-imidazol-1- yl)-4-methyl-4,5,6,7- tetrahydro-2H- pyrazolo[4,3-c]pyridine- 5-carbonyl)-5- (tetrahydro-2H-pyran-4- yl)-1H-indol-1-yl)-2- methylcyclopropyl)- 1,2,4-oxadiazol-5(4H)- one | 894.2 |
| 134 | 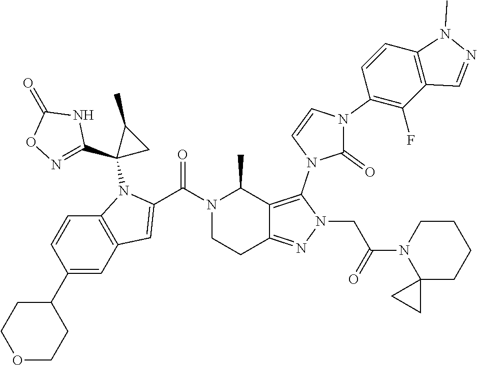 [SMILES: C[C@H]1c2c(nn(CC(=O)N3CCCCC34CC4)c2-n2ccn(-c3ccc4c(cnn4C)c3F)c2=O)CCN1C(=O)c1cc2cc(C3CCOCC3)ccc2n1[C@@]1(c2noc(=O)[nH]2)C[C@@H]1C] | 3-((1S,2S)-1-(2-((S)-3-(3- (4-fluoro-1-methyl-1H- indazol-5-yl)-2-oxo-2,3- dihydro-1H-imidazol-1- yl)-4-methyl-2-(2-oxo-2- (4-azaspiro[2.5]octan-4- yl)ethyl)-4,5,6,7- tetrahydro-2H- pyrazolo[4,3-c]pyridine- 5-carbonyl)-5- (tetrahydro-2H-pyran-4- yl)-1H-indol-1-yl)-2- methylcyclopropyl)- 1,2,4-oxadiazol-5(4H)- one | 884.4 |
| 135 | 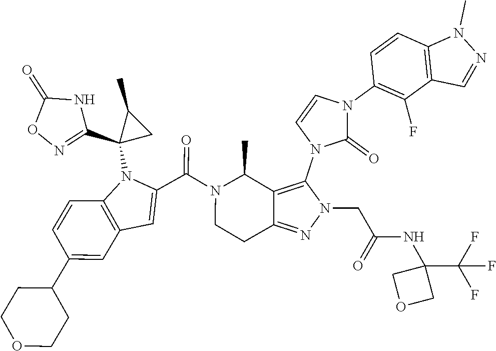 [SMILES: C[C@H]1c2c(nn(CC(=O)NC3(C(F)(F)F)COC3)c2-n2ccn(-c3ccc4c(cnn4C)c3F)c2=O)CCN1C(=O)c1cc2cc(C3CCOCC3)ccc2n1[C@@]1(c2noc(=O)[nH]2)C[C@@H]1C] | 2-((S)-3-(3-(4-fluoro-1- methyl-1H-indazol-5-yl)- 2-oxo-2,3-dihydro-1H- imidazol-1-yl)-4-methyl- 5-(1-((1S,2S)-2-methyl-1- (5-oxo-4,5-dihydro-1,2,4- oxadiazol-3- yl)cyclopropyl)-5- (tetrahydro-2H-pyran-4- yl)-1H-indole-2- carbonyl)-4,5,6,7- tetrahydro-2H- pyrazolo[4,3-c]pyridin-2- yl)-N-(3- (trifluoromethyl)oxetan- 3-yl)acetamide | 914.6 |
| 136 | 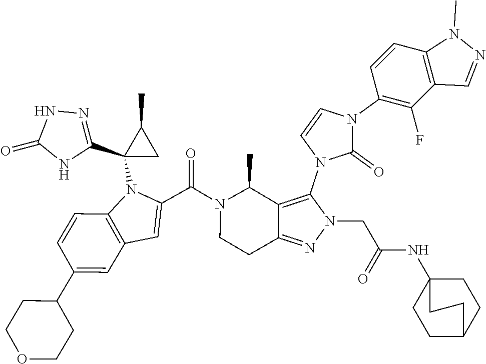 [SMILES: C[C@H]1c2c(nn(CC(=O)NC34CCC(CC3)CC4)c2-n2ccn(-c3ccc4c(cnn4C)c3F)c2=O)CCN1C(=O)c1cc2cc(C3CCOCC3)ccc2n1[C@@]1(c2n[nH]c(=O)[nH]2)C[C@@H]1C] | N-(bicyclo[2.2.2]octan-1- yl)-2-((S)-3-(3-(4-fluoro- 1-methyl-1H-indazol-5- yl)-2-oxo-2,3-dihydro- 1H-imidazol-1-yl)-4- methyl-5-(1-((1S,2S)-2- methyl-1-(5-oxo-4,5- dihydro-1,2,4-oxadiazol- 3-yl)cyclopropyl)-5- (tetrahydro-2H-pyran-4- yl)-1H-indole-2- carbonyl)-4,5,6,7- tetrahydro-2H- pyrazolo[4,3-c]pyridin-2- yl)acetamide | 898.6 |

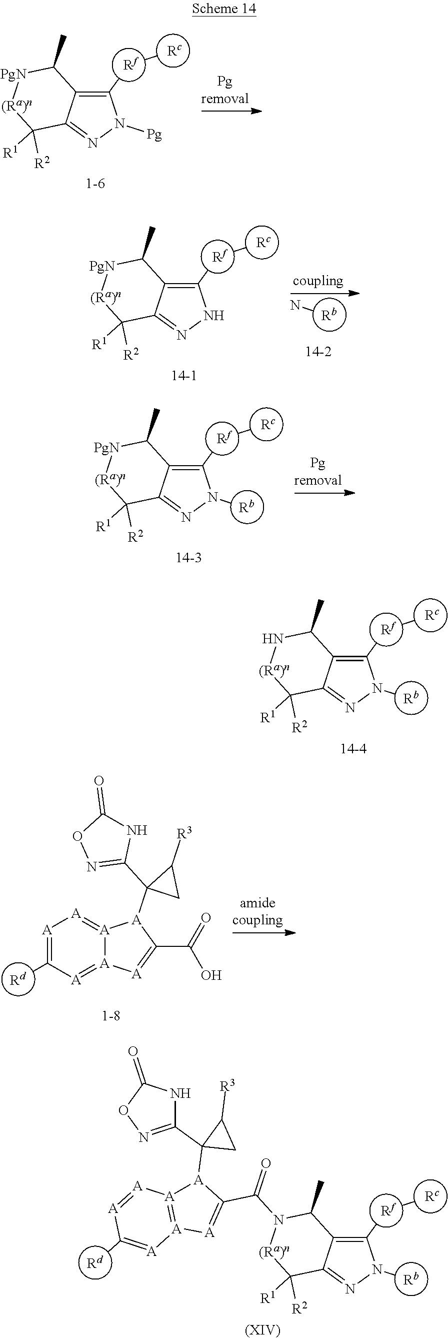

Compounds of formula (XIV) are prepared from intermediate 1-6 by removal of the protecting group on the nitrogen atom of the pyrazolyl moiety (e.g., benzyl group via hydrogenolysis) to provide azole 14-1. Subsequently, 14-1 is coupled with halides or other partners such as boronic acids, sulfonates or sulfones (14-2) via transition metal catalyzed cross-coupling (e.g., Pd—or Cu-catalysis), SNAr or alkylation (e.g., basic) conditions to furnish intermediate 14-3. Removal of the protecting group (e.g., acidic conditions) of 14-3 provides amine 14-4 that is then coupled with carboxylic acid 1-8 to provide compounds of formula (XIV).

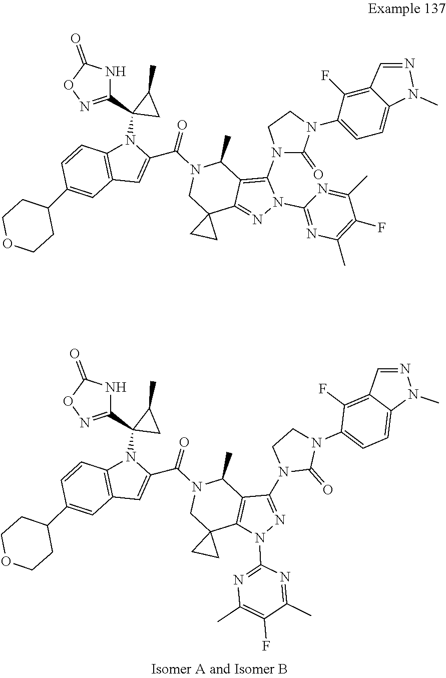 [SMILES: Cc1nc(-n2nc3c(c2N2CCN(c4ccc5c(cnn5C)c4F)C2=O)[C@H](C)N(C(=O)c2cc4cc(C5CCOCC5)ccc4n2[C@@]2(c4noc(=O)[nH]4)C[C@@H]2C)CC32CC2)nc(C)c1F ; Cc1nc(-n2nc(N3CCN(c4ccc5c(cnn5C)c4F)C3=O)c3c2C2(CC2)CN(C(=O)c2cc4cc(C5CCOCC5)ccc4n2[C@@]2(c4noc(=O)[nH]4)C[C@@H]2C)[C@H]3C)nc(C)c1F]

3-((1S,2S)-1-(2-((S)-3′-(3-(4-fluoro-1-methyl-1H-indazol-5-yl)-2-oxoimidazolidin-1-yl)-2′-(5-fluoro-4,6-dimethylpyrimidin-2-yl)-4′-methyl-2′,4′,5′,6′-tetrahydrospiro[cyclopropane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one and 3-((1S,2S)-1-(2-((S)-3′-(3-(4-fluoro-1-methyl-1H-indazol-5-yl)-2-oxoimidazolidin-1-yl)-1′-(5-fluoro-4,6-dimethylpyrimidin-2-yl)-4′-methyl-1′,4′,5′,6′-tetrahydrospiro[cyclopropane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one (scheme 14)

### Step 1: tert-butyl (S)-3′-(3-(4-fluoro-1-methyl-1H-indazol-5-yl)-2-oxoimidazolidin-1-yl)-4′-methyl-2′,4′-dihydrospiro[cyclopropane-1,7′-pyrazolo[4,3-c]pyridine]-5′(6′H)-carboxylate

To a 4 mL vial containing tert-butyl (S)-2′-benzyl-3′-(3-(4-fluoro-1-methyl-1H-indazol-5-yl)-2-oxo-2,3-dihydro-1H-imidazol-1-yl)-4′-methyl-2′,4′-dihydrospiro[cyclopropane-1,7′-pyrazolo[4,3-c]pyridine]-5′(6′H)-carboxylate (0.314 g, 538 mol) was added Pd/C Noblyst® P1070 (115 mg, 53.8 mol) followed by MeOH (6.28 mL). The vial was sealed, and a balloon of hydrogen gas was added. Hydrogen gas was bubbled through via subsurface bubbling for 20 seconds. Once the atmosphere exchange was complete, stirred at 60° C. for 15 h. The reaction was filtered over Celite® and the filter cake washed with MeOH. The filtrate was concentrated and the residue purified by silica gel chromatography (0-100% 3:1 EtOAc:EtOH in hexane) to provide the title compound. MS (ESI) m/z: 496.2 [M+H]. ¹H NMR (500 MHz, MeOD) δ 8.13 (s, 1H), 7.54-7.47 (m, 1H), 7.45 (d, J=8.7 Hz, 1H), 5.66 (s, 1H), 4.26 (s, 1H), 4.12 (s, 3H), 4.05 (dd, J=18.3, 10.9 Hz, 2H), 3.89 (d, J=8.7 Hz, 1H), 3.55 (s, 2H), 1.48 (s, 9H), 1.37 (d, J=6.3 Hz, 4H), 1.09 (s, 1H), 1.00 (s, 1H), 0.84 (s, 1H).

### Step 2: tert-butyl (S)-3′-(3-(4-fluoro-1-methyl-1H-indazol-5-yl)-2-oxoimidazolidin-1-yl)-2′-(5-fluoro-4,6-dimethylpyrimidin-2-yl)-4′-methyl-2′,4′-dihydrospiro[cyclopropane-1,7′-pyrazolo[4,3-c]pyridine]-5′(6′H)-carboxylate or tert-butyl (S)-3′-(3-(4-fluoro-1-methyl-1H-indazol-5-yl)-2-oxoimidazolidin-1-yl)-1′-(5-fluoro-4,6-dimethylpyrimidin-2-yl)-4′-methyl-1′,4′-dihydrospiro[cyclopropane-1,7′-pyrazolo[4,3-c]pyridine]-5′(6′H)-carboxylate

To a mixture of tert-butyl (S)-3′-(3-(4-fluoro-1-methyl-1H-indazol-5-yl)-2-oxoimidazolidin-1-yl)-4′-methyl-2′,4′-dihydrospiro[cyclopropane-1,7′-pyrazolo[4,3-c]pyridine]-5′(6′H)-carboxylate (58 mg, 0.12 mmol) in DMA (1.7 mL) was added 2-chloro-5-fluoro-4,6-dimethylpyrimidine (56 mg, 0.35 mmol) followed by potassium tert-butoxide (20 mg, 0.18 mmol). The reaction mixture was heated to 100° C. for 15 h. The reaction was cooled to RT and diluted with EtOAc and water. The organic layer was extracted 2× with EtOAc. The combined organic layer was dried with magnesium sulfate, filtered, and concentrated under reduced pressure. The crude mixture was purified by silica gel chromatography (0-100% 3:1 EtOAc:EtOH in hexane) to afford the title compounds as separated regioisomers. Data for isomer A (faster eluting regioisomer): MS (ESI) m/z: 620.6 [M+H]. Data for isomer B (slower eluting regioisomer): tert-butyl (S)-3′-(3-(4-fluoro-1-methyl-1H-indazol-5-yl)-2-oxoimidazolidin-1-yl)-1′-(5-fluoro-4,6-dimethylpyrimidin-2-yl)-4′-methyl-1′,4′-dihydrospiro[cyclopropane-1,7′-pyrazolo[4,3-c]pyridine]-5′(6′H)-carboxylate (slower eluting regioisomer) MS (ESI) m/z: 620.6 [M+H].

### Step 3: (S)-1-(4-fluoro-1-methyl-1H-indazol-5-yl)-3-(2′-(5-fluoro-4,6-dimethylpyrimidin-2-yl)-4′-methyl-2′,4′,5,6′-tetrahydrospiro[cyclopropane-1,7′-pyrazolo[4,3-c]pyridin]-3′-yl)imidazolidin-2-one or (S)-1-(4-fluoro-1-methyl-1H-indazol-5-yl)-3-(1′-(5-fluoro-4,6-dimethylpyrimidin-2-yl)-4′-methyl-1′,4′,5′,6′-tetrahydrospiro[cyclopropane-1,7′-pyrazolo[4,3-c]pyridin]-3′-yl)imidazolidin-2-one

Procedure for isomer A: To a mixture of tert-butyl (S)-3′-(3-(4-fluoro-1-methyl-1H-indazol-5-yl)-2-oxoimidazolidin-1-yl)-2′-(5-fluoro-4,6-dimethylpyrimidin-2-yl)-4′-methyl-2′,4′-dihydrospiro[cyclopropane-1,7′-pyrazolo[4,3-c]pyridine]-5′(6′H)-carboxylate or tert-butyl (S)-3′-(3-(4-fluoro-1-methyl-1H-indazol-5-yl)-2-oxoimidazolidin-1-yl)-1′-(5-fluoro-4,6-dimethylpyrimidin-2-yl)-4′-methyl-1′,4′-dihydrospiro[cyclopropane-1,7′-pyrazolo[4,3-c]pyridine]-5′(6′H)-carboxylate (faster eluting isomer from previous step) (16 mg, 0.026 mmol) in DCM (0.5 mL) was added HCl in dioxane (4M, 65 mL, 0.26 mmol). The reaction was stirred for 15 h at room temperature. Additional HCl in dioxane (4M, 97 mL, 0.39 mmol) was added and the reaction stirred at RT for 6 h. The reaction was concentrated under reduced pressure to give the title compound and used in the next step without purification. MS (ESI) m/z: 520.2 [M+H]+. Procedure for isomer B: To a mixture of a mixture of tert-butyl (S)-3′-(3-(4-fluoro-1-methyl-1H-indazol-5-yl)-2-oxoimidazolidin-1-yl)-2′-(5-fluoro-4,6-dimethylpyrimidin-2-yl)-4′-methyl-2′,4′-dihydrospiro[cyclopropane-1,7′-pyrazolo[4,3-c]pyridine]-5′(6′H)-carboxylate or tert-butyl (S)-3′-(3-(4-fluoro-1-methyl-1H-indazol-5-yl)-2-oxoimidazolidin-1-yl)-1′-(5-fluoro-4,6-dimethylpyrimidin-2-yl)-4′-methyl-1′,4′-dihydrospiro[cyclopropane-1,7′-pyrazolo[4,3-c]pyridine]-5′(6′H)-carboxylate (slower eluting isomer from previous step) (11 mg, 0.018 mmol) in DCM (0.5 mL) was added HCl in dioxane (4M, 44 mL, 0.18 mmol). The reaction was stirred for 15 h at room temperature. Additional HCl in dioxane (4M, 67 mL, 0.27 mmol) was added and the reaction stirred at RT for 6 h. The reaction was concentrated under reduced pressure to give the title compound used in the next step without purification. MS (ESI) m/z: 520.2 [M+H]+.

### Step 4: 3-((1S,2S)-1-(2-((S)-3′-(3-(4-fluoro-1-methyl-1H-indazol-5-yl)-2-oxoimidazolidin-1-yl)-2′-(5-fluoro-4,6-dimethylpyrimidin-2-yl)-4′-methyl-2′,4′,5′,6′-tetrahydrospiro[cyclopropane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one and 3-((1S,2S)-1-(2-((S)-3′-(3-(4-fluoro-1-methyl-1H-indazol-5-yl)-2-oxoimidazolidin-1-yl)-1′-(5-fluoro-4,6-dimethylpyrimidin-2-yl)-4′-methyl-1′,4′,5′,6′-tetrahydrospiro[cyclopropane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one

Procedure for isomer A: To a stirred solution of (S)-1-(4-fluoro-1-methyl-1H-indazol-5-yl)-3-(2′-(5-fluoro-4,6-dimethylpyrimidin-2-yl)-4′-methyl-2′,4′,5′,6′-tetrahydrospiro[cyclopropane-1,7′-pyrazolo[4,3-c]pyridin]-3′-yl)imidazolidin-2-one or (S)-1-(4-fluoro-1-methyl-1H-indazol-5-yl)-3-(1′-(5-fluoro-4,6-dimethylpyrimidin-2-yl)-4′-methyl-1′,4′,5′,6′-tetrahydrospiro[cyclopropane-1,7′-pyrazolo[4,3-c]pyridin]-3′-yl)imidazolidin-2-one (11 mg, 0.018 mmol) in DMF (0.5 mL) was added 1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carboxylic acid (16 mg, 0.026 mmol) in DMF (0.5 mL) was added 1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carboxylic acid (17 mg, 0.039 mmol), HATU (13 mg, 0.034 mmoL) and DIPEA (45 mL, 0.26 mmol). The reaction was stirred at RT for 15 h. The reaction mixture was filtered and purified by reverse phase chromatography (column: Waters™ CSH C18, 30×150 mm; method: 50-70% MeCN/water, with 0.1% Formic Acid) to provide the title compound. ¹H NMR (600 MHz, DMSO) δ 8.18-8.22 (m, 2H), 7.51-7.56 (m, 1H), 7.47-7.39 (m, 2H), 7.16 (d, J=8.5 Hz, 1H), 5.84-5.90 (m, 1H), 4.07 (s, 3H), 3.97 (d, J=9.5 Hz, 2H), 3.43-3.51 (m, 1H), 2.78-2.86 (m, 1H), 1.70-1.79 (m, 3H), 1.57-1.63 (m, 1H), 1.45-1.55 (m, 3H), 1.24-1.30 (m, 4H). 0.78-0.90 (m, 1H), 0.62-0.74 (m, 1H), 0.46-0.57 (m, 1H). MS (ESI) m/z 885.8. Procedure for isomer B: To a stirred solution of (S)-1-(4-fluoro-1-methyl-1H-indazol-5-yl)-3-(2′-(5-fluoro-4,6-dimethylpyrimidin-2-yl)-4′-methyl-2′,4′,5′,6′-tetrahydrospiro[cyclopropane-1,7′-pyrazolo[4,3-c]pyridin]-3′-yl)imidazolidin-2-one or (S)-1-(4-fluoro-1-methyl-1H-indazol-5-yl)-3-(1′-(5-fluoro-4,6-dimethylpyrimidin-2-yl)-4′-methyl-1′,4′,5′,6′-tetrahydrospiro[cyclopropane-1,7′-pyrazolo[4,3-c]pyridin]-3′-yl)imidazolidin-2-one (11 mg, 0.018 mmol) in DMF (0.5 mL) was added 1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carboxylic acid (12 mg, 0.027 mmol), HATU (8.9 mg, 0.023 mmoL) and DIPEA (31 mL, 0.18 mmol). The reaction was stirred at RT for 15 h. The reaction mixture was filtered and purified by reverse phase chromatography (Column: Waters™ CSH C18, 30×150 mm; Method: 50-70% MeCN/Water, with 0.1% Formic Acid) to provide the title compound. MS (ESI) m/z 886.0.

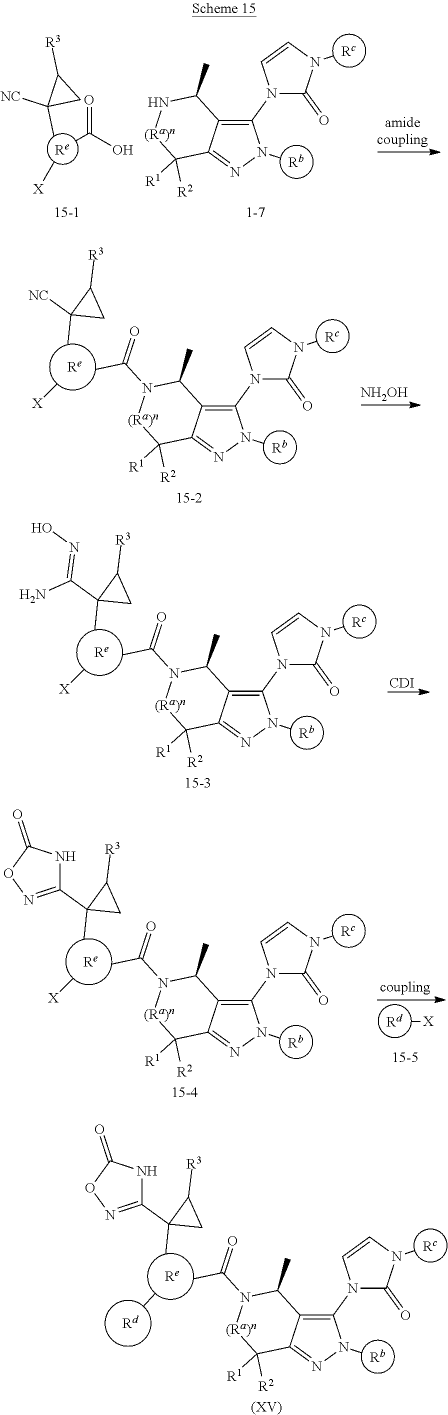

Compounds of formula (XV) are prepared from acid 15-1 by amide coupling with amine 1-7 to provide intermediate 15-2. Subsequently, 15-2 is treated with hydroxylamine to provide 15-3 that is then cyclized by treatment with CDI to provide 1,2,4-oxadiazolone 15-4. Then, 15-4 is coupled with boronic acids, boronates, sulfonates, halides, carboxylic acids, alcohols and other suitable partners (15-5) via transition metal catalyzed cross-coupling (e.g., Pd- or Cu-catalysis or Photoredox/Ni dual catalysis) to provide compounds of formula (XV).

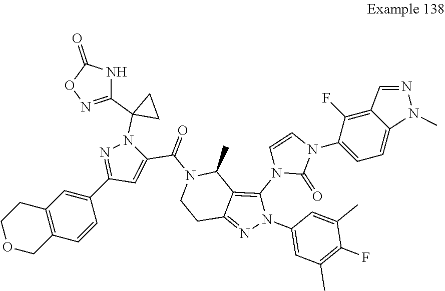 [SMILES: Cc1cc(-n2nc3c(c2-n2ccn(-c4ccc5c(cnn5C)c4F)c2=O)[C@H](C)N(C(=O)c2cc(-c4ccc5c(c4)CCOC5)nn2C2(c4noc(=O)[nH]4)CC2)CC3)cc(C)c1F]

### (S)-3-(1-(5-(3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-3-(isochroman-6-yl)-1H-pyrazol-1-yl)cyclopropyl)-1,2,4-oxadiazol-5(4H)-one (Scheme 15)

### Step 1: (S)-1-(5-(3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-3-(isochroman-6-yl)-1H-pyrazol-1-yl)cyclopropane-1-carbonitrile

To a solution of (S)-1-(4-fluoro-1-methyl-1H-indazol-5-yl)-3-(2-(4-fluoro-3,5-dimethylphenyl)-4-methyl-4,5,6,7-tetrahydro-2H-pyrazolo[4,3-c]pyridin-3-yl)-1,3-dihydro-2H-imidazol-2-one hydrochloride (48.6 mg, 92.4 mol), 3-bromo-1-(1-cyanocyclopropyl)-1H-pyrazole-5-carboxylic acid (28.4 mg, 111 mol) in CH₂Cl₂ (2.00 mL) was added diisopropylethylamine (95.6 mg, 739 mol) (stirred 1-2 min) then propylphosphonic anhydride (88.2 mg, 277 μmol). The mixture was stirred overnight at rt. Then, sat aq. NaHCO₃ (10 mL) was added, and the mixture was extracted with EtOAc (4×10 mL). The combined organic layers were dried over Na₂SO₄ and concentrated under reduced pressure. The crude mixture was purified by SiO₂ chromatography (0-80% EtOAc/Hex) to afford the title compound. MS (ESI) m/z 727.3, 729.3 [M+1]+

### Step 2: (S,Z)-1-(5-(3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-3-(isochroman-6-yl)-1H-pyrazol-1-yl)-N′-hydroxycyclopropane-1-carboximidamide or (S,E)-1-(5-(3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-3-(isochroman-6-yl)-1H-pyrazol-1-yl)-N′-hydroxycyclopropane-1-carboximidamide

A solution of (S)-1-(3-bromo-5-(3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-1H-pyrazol-1-yl)cyclopropane-1-carbonitrile (544 mg, 0.748 mmol), sodium bicarbonate (330 mg, 3.93 mmol) and hydroxylamine hydrochloride (260 mg, 0.15 mL, 3.74 mmol) in ethanol (10 mL) was stirred at 75° C. for 2 h. The reaction mixture was cooled to rt, filtered and the filtrate was concentrated under reduced pressure to afford the title compound. MS (ESI) m/z 760.4, 762.4 [M+1]+

### Step 3: (S)-3-(1-(5-(3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-3-(isochroman-6-yl)-1H-pyrazol-1-yl)cyclopropyl)-1,2,4-oxadiazol-5(4H)-one

To a mixture of methyl (S,Z)-1-(3-bromo-5-(3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-1H-pyrazol-1-yl)-N′-hydroxycyclopropane-1-carboximidamide (690 mg, 0.907 mmol) and 1,8-diazabicyclo[5.4.0]undec-7-ene (345 mg, 2.27 mmol) in DMSO (4 mL) was added N,N′-carbonyldiimidazole (368 mg, 2.27 mmol). The reaction mixture was stirred at 23° C. for 1 h. Then, the mixture was quenched by the addition of H₂O (8 mL) and extracted with EtOAc (10×20 mL). The combined organic layers were dried over Na₂SO₄, filtered, and concentrated under reduced pressure. The crude residue was purified by SiO₂ chromatography (0 to 15% MeOH/DCM) then reverse-phase HPLC (15 to 40% MeCN/H₂O) to afford the title compound. MS (ESI) m/z 786.3, 788.3 [M+1]+

### Step 4: (S)-3-(1-(5-(3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-3-(isochroman-6-yl)-1H-pyrazol-1-yl)cyclopropyl)-1,2,4-oxadiazol-5(4H)-one

A mixture of 3-(1-(3-bromo-5-((4S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-1H-pyrazol-1-yl)cyclopropyl)-1,2,4-oxadiazol-5(4H)-one (20 mg, 0.025 mmol), 2-(isochroman-6-yl)-4,4,5,5-tetramethyl-1,3,2-dioxaborolane (7.9 mg, 31 μmol), K₃PO₄ (22 mg, 0.10 mmol), and [1,1′-bis(di-tert-butylphosphino)ferrocene]dichloropalladium(II) (3.3 mg, 5.1 μmol) in DMF (0.85 mL) was heated to 70° C. under nitrogen atmosphere for 18 h. The reaction mixture was then filtered and purified by reverse-phase HPLC (40% to 90% MeCN/H₂O) to afford the title compound. 1H NMR (mixture of rotamers): (500 MHz, DMSO-d6) δ 8.31 (s, 2H), 8.24-8.12 (m, 3H), 7.70-7.61 (m, 7H), 7.54-7.33 (m, 4H), 7.17 (d, J=6.2 Hz, 6H), 7.13-6.84 (m, 12H), 6.54 (s, 3H), 5.76 (s, 1H), 5.56 (d, J=6.7 Hz, 2H), 5.21 (s, 1H), 4.68 (d, J=24.5 Hz, 7H), 4.28-4.04 (m, 11H), 3.94-3.72 (m, 7H), 3.02-2.90 (m, 3H), 2.88-2.80 (m, 4H), 2.81-2.60 (m, 6H), 2.50 (s, 13H), 2.32-2.16 (m, 17H), 1.78 (d, J=35.7 Hz, 6H), 1.66-1.47 (m, 6H), 1.39 (d, J=6.2 Hz, 3H), 1.21 (dd, J=43.7, 6.9 Hz, 7H). MS (ESI) m/z 840.6 [M+1]

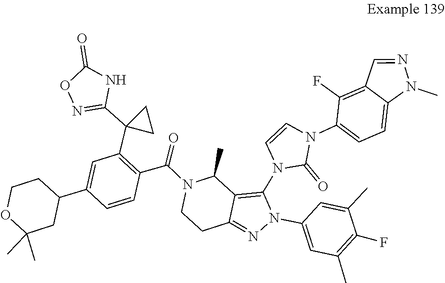 [SMILES: Cc1cc(-n2nc3c(c2-n2ccn(-c4ccc5c(cnn5C)c4F)c2=O)[C@H](C)N(C(=O)c2ccc(C4CCOC(C)(C)C4)cc2C2(c4noc(=O)[nH]4)CC2)CC3)cc(C)c1F]

### 3-(1-(5-((S)-2,2-dimethyltetrahydro-2H-pyran-4-yl)-2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)phenyl)cyclopropyl)-1,2,4-oxadiazol-5(4H)-one and 3-(1-(5-((R)-2,2-dimethyltetrahydro-2H-pyran-4-yl)-2-((S)-3-(3-(4-fluoro-1-methyl-H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)phenyl)cyclopropyl)-1,2,4-oxadiazol-5(4H)-one (scheme 15)

### Steps 1-3: (S)-3-(1-(5-bromo-2-(3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)phenyl)cyclopropyl)-1,2,4-oxadiazol-5(4H)-one

These steps were performed in a similar fashion as described for example 138 to provide the title compound. MS: LCMS (ESI) m/z 796.4.

### Step 4: 3-(1-(5-((S)-2,2-dimethyltetrahydro-2H-pyran-4-yl)-2-((S)-3-(3-(4-fluoro-1-methyl-1H-indazol-5-yl)-2-oxo-2,3-dihydro-1H-imidazol-1-yl)-2-(4-fluoro-3,5-dimethylphenyl-4-methyl-4,5,6,7-tetrahydro-2H-pyrazolo[4,3-c]pyridine-5-carbonyl)phenyl)cyclopropyl)-1,2,4-oxadiazol-5(4H)-one and 3-(1-(5-((R)-2,2-dimethyltetrahydro-2H-pyran-4-yl)-2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)phenyl)cyclopropyl)-1,2,4-oxadiazol-5(4H)-one

In a nitrogen-filled glove box, a 1 dram vial was charged with a stir bar, (S)-3-(1-(5-bromo-2-(3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)phenyl)cyclopropyl)-1,2,4-oxadiazol-5(4H)-one (25 mg, 0.031 mmol), 2,2-dimethyl-2H-pyran-4-carboxcylic acid (7.45 mg, 0.047 mmol), phthalimide (4.62 mg, 0.031 mmol), tetramethly-phenanthroline nickel di-bromide (2.95 mg, 6.28 3 mol), (Ir[dF(CF₃)ppy]₂(dtbpy))PF₆ (0.704 mg, 0.628 μmol), DMSO (1 ml), and 2-tert-butylL-1,1,3,3-tetramethylquanidine (0.016 ml, 0.078 mmol). The vial was sealed and stirred at 1000 rpm under irradiation with 450 nm LEDs at rt for 18 h. After the reaction was complete, the crude residue was purified by prep-HPLC (450 to 800 MeCN/pH 10 aqueous ammonium hydroxide gradient). ¹H NMR (499 MHz, CDCl₃) δ 11.43 (s, 1H), 8.15 (s, 1H), 7.60-7.38 (m, 2H), 7.23-7.04 (m, 2H), 6.63 (s, 1H), 6.50-6.26 (m, 2H), 6.15-5.77 (m, 3H), 4.14 (s, 3H), 3.96-3.65 (m, 4H), 3.48 (s, 1H), 3.06-2.94 (m, 2H), 2.94-2.83 (m, 1H), 2.44-2.22 (m, 5H), 2.06 (s, 1H), 1.84-1.68 (m, 4H), 1.58-1.50 (m, 3H), 1.47-1.24 (m, 6H), 1.18 (s, 2H), 0.94 (s, 1H). MS: LCMS (ESI) m/z=830.8. The following examples in table 12 were prepared according to scheme 15 using the procedures outlined in the synthesis of examples 138 or 139.

TABLE 12

| | | | MS |
|---|---|---|---|
| Ex. | Structure | Name | (M + 1) |
| 140 | 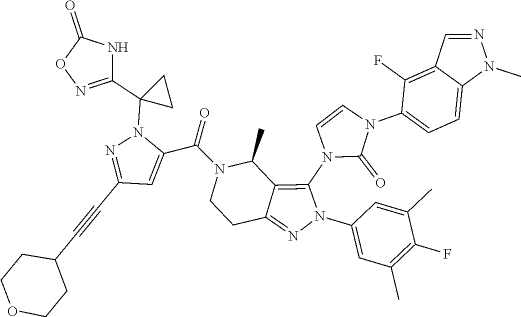 [SMILES: Cc1cc(-n2nc3c(c2-n2ccn(-c4ccc5c(cnn5C)c4F)c2=O)[C@H](C)N(C(=O)c2cc(C#CC4CCOCC4)nn2C2(c4noc(=O)[nH]4)CC2)CC3)cc(C)c1F] | (S)-3-(1-(5-(3-(3-(4-fluoro- 1-methyl-1H-indazol-5-yl)- 2-oxo-2,3-dihydro-1H- imidazol-1-yl)-2-(4-fluoro- 3,5-dimethylphenyl)-4- methyl-4,5,6,7-tetrahydro- 2H-pyrazolo[4,3- c]pyridine-5-carbonyl)-3- ((tetrahydro-2H-pyran-4- yl)ethynyl)-1H-pyrazol-1- yl)cyclopropyl)-1,2,4- oxadiazol-5(4H)-one | 816.3 |
| 141 | 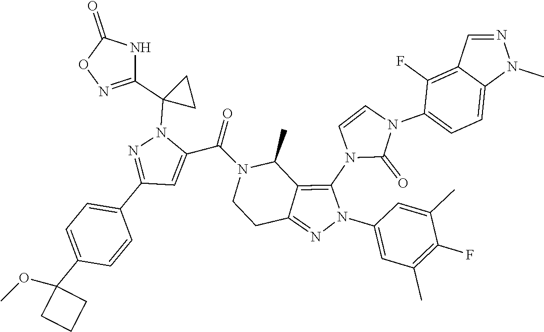 | (S)-3-(1-(5-(3-(3-(4-fluoro- 1-methyl-1H-indazol-5-yl)- 2-oxo-2,3-dihydro-1H- imidazol-1-yl)-2-(4-fluoro- 3,5-dimethylphenyl)-4- methyl-4,5,6,7-tetrahydro- 2H-pyrazolo[4,3- c]pyridine-5-carbonyl)-3- (4-(1- methoxycyclobutyl)phenyl)- 1H-pyrazol-1- yl)cyclopropyl)-1,2,4- oxadiazol-5(4H)-one | 868.4 |
| 142 | 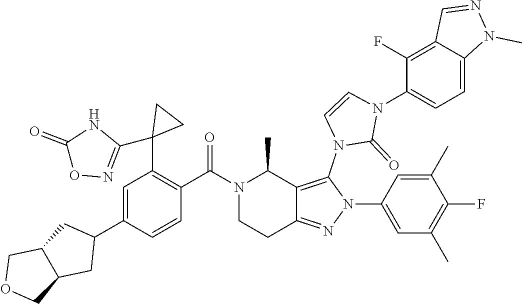 [SMILES: Cc1cc(-n2nc3c(c2-n2ccn(-c4ccc5c(cnn5C)c4F)c2=O)[C@H](C)N(C(=O)c2ccc(C4C[C@H]5COC[C@@H]5C4)cc2C2(c4noc(=O)[nH]4)CC2)CC3)cc(C)c1F] and 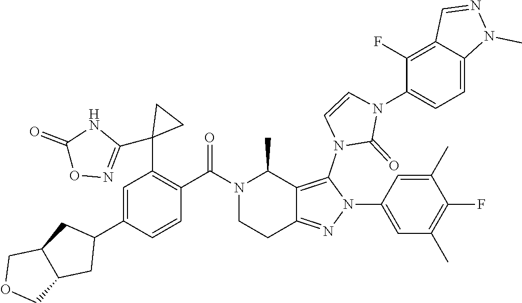 [SMILES: Cc1cc(-n2nc3c(c2-n2ccn(-c4ccc5c(cnn5C)c4F)c2=O)[C@H](C)N(C(=O)c2ccc(C4C[C@@H]5COC[C@H]5C4)cc2C2(c4noc(=O)[nH]4)CC2)CC3)cc(C)c1F] | 3-(1-(2-((S)-3-(3-(4-fluoro- 1-methyl-1H-indazol-5-yl)- 2-oxo-2,3-dihydro-1H- imidazol-1-yl)-2-(4-fluoro- 3,5-dimethylphenyl)-4- methyl-4,5,6,7-tetrahydro- 2H-pyrazolo[4,3- c]pyridine-5-carbonyl)-5- ((3aR,6aR)-hexahydro-1H- cyclopenta[c]furan-5- yl)phenyl)cyclopropyl)- 1,2,4-oxadiazol-5(4H)-one and 3-(1-(2-((S)-3-(3-(4- fluoro-1-methyl-1H- indazol-5-yl)-2-oxo-2,3- dihydro-1H-imidazol-1-yl)- 2-(4-fluoro-3,5- dimethylphenyl)-4-methyl- 4,5,6,7-tetrahydro-2H- pyrazolo[4,3-c]pyridine-5- carbonyl)-5-((3aS,6aS)- hexahydro-1H- cyclopenta[c]furan-5- yl)phenyl)cyclopropyl)- 1,2,4-oxadiazol-5(4H)-one | 828.7 |
| 143 | 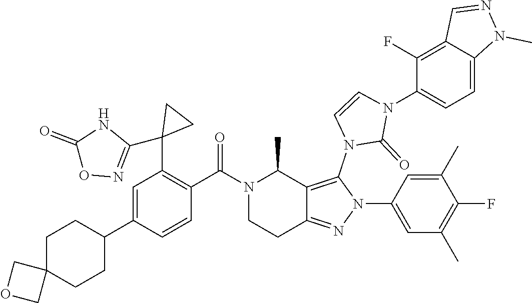 [SMILES: Cc1cc(-n2nc3c(c2-n2ccn(-c4ccc5c(cnn5C)c4F)c2=O)[C@H](C)N(C(=O)c2ccc(C4CCC5(CC4)COC5)cc2C2(c4noc(=O)[nH]4)CC2)CC3)cc(C)c1F] | (S)-3-(1-(2-(3-(3-(4-fluoro- 1-methyl-1H-indazol-5-yl)- 2-oxo-2,3-dihydro-1H- imidazol-1-yl)-2-(4-fluoro- 3,5-dimethylphenyl)-4- methyl-4,5,6,7-tetrahydro- 2H-pyrazolo[4,3- c]pyridine-5-carbonyl)-5- (2-oxaspiro[3.5]nonan-7- yl)phenyl)cyclopropyl)- 1,2,4-oxadiazol-5(4H)-one, | 842.8 |
| 144 | 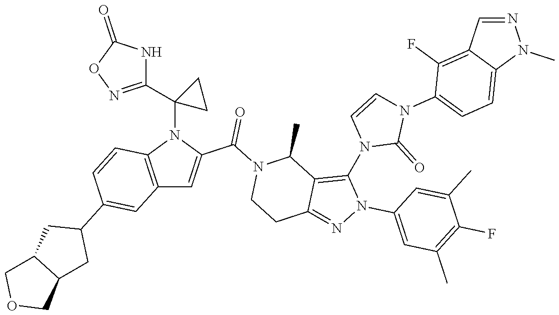 [SMILES: Cc1cc(-n2nc3c(c2-n2ccn(-c4ccc5c(cnn5C)c4F)c2=O)[C@H](C)N(C(=O)c2cc4cc(C5C[C@H]6COC[C@@H]6C5)ccc4n2C2(c4noc(=O)[nH]4)CC2)CC3)cc(C)c1F] and 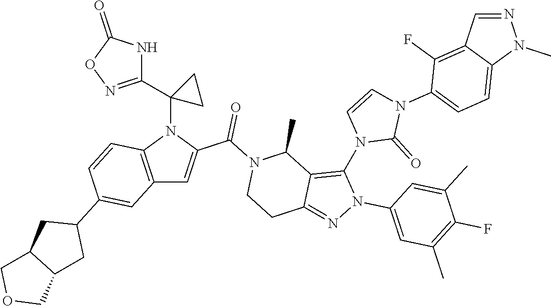 [SMILES: Cc1cc(-n2nc3c(c2-n2ccn(-c4ccc5c(cnn5C)c4F)c2=O)[C@H](C)N(C(=O)c2cc4cc(C5C[C@@H]6COC[C@H]6C5)ccc4n2C2(c4noc(=O)[nH]4)CC2)CC3)cc(C)c1F] | 3-(1-(2-((S)-3-(3-(4-fluoro- 1-methyl-1H-indazol-5-yl)- 2-oxo-2,3-dihydro-1H- imidazol-1-yl)-2-(4-fluoro- 3,5-dimethylphenyl)-4- methyl-4,5,6,7-tetrahydro- 2H-pyrazolo[4,3- c]pyridine-5-carbonyl)-5- ((3aR,6aR)-hexahydro-1H- cyclopenta[c]furan-5-yl)- 1H-indol-1- yl)cyclopropyl)-1,2,4- oxadiazol-5(4H)-one and 3- (1-(2-((S)-3-(3-(4-fluoro-1- methyl-1H-indazol-5-yl)-2- oxo-2,3-dihydro-1H- imidazol-1-yl)-2-(4-fluoro- 3,5-dimethylphenyl)-4- methyl-4,5,6,7-tetrahydro- 2H-pyrazolo[4,3- c]pyridine-5-carbonyl)-5- ((3aS,6aS)-hexahydro-1H- cyclopenta[c]furan-5-yl)- 1H-indol-1- yl)cyclopropyl)-1,2,4- oxadiazol-5(4H)-one | 867.7 |
| 145 | 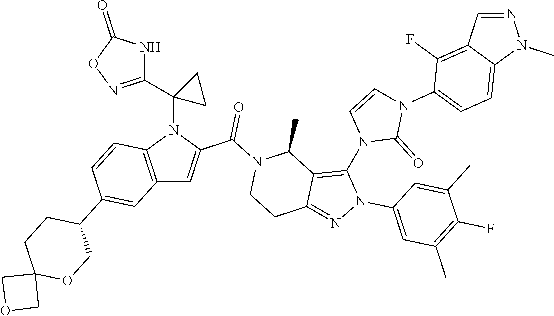 [SMILES: Cc1cc(-n2nc3c(c2-n2ccn(-c4ccc5c(cnn5C)c4F)c2=O)[C@H](C)N(C(=O)c2cc4cc([C@H]5CCC6(COC6)OC5)ccc4n2C2(c4noc(=O)[nH]4)CC2)CC3)cc(C)c1F] and 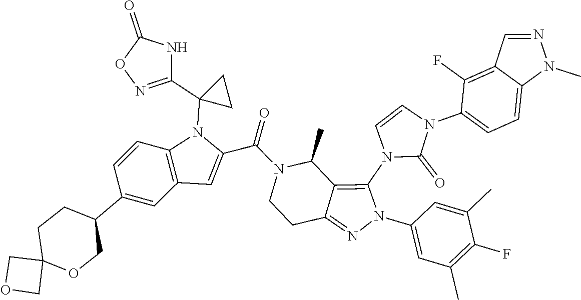 [SMILES: Cc1cc(-n2nc3c(c2-n2ccn(-c4ccc5c(cnn5C)c4F)c2=O)[C@H](C)N(C(=O)c2cc4cc([C@@H]5CCC6(COC6)OC5)ccc4n2C2(c4noc(=O)[nH]4)CC2)CC3)cc(C)c1F] | 3-(1-(2-((S)-3-(3-(4-fluoro- 1-methyl-1H-indazol-5-yl)- 2-oxo-2,3-dihydro-1H- imidazol-1-yl)-2-(4-fluoro- 3,5-dimethylphenyl)-4- methyl-4,5,6,7-tetrahydro- 2H-pyrazolo[4,3- c]pyridine-5-carbonyl)-5- ((R)-2,5- dioxaspiro[3.5]nonan-7-yl)- 1H-indol-1- yl)cyclopropyl)-1,2,4- oxadiazol-5(4H)-one and 3- (1-(2-((S)-3-(3-(4-fluoro-1- methyl-1H-indazol-5-yl)-2- oxo-2,3-dihydro-1H- imidazol-1-yl)-2-(4-fluoro- 3,5-dimethylphenyl)-4- methyl-4,5,6,7-tetrahydro- 2H-pyrazolo[4,3- c]pyridine-5-carbonyl)-5- ((S)-2,5- dioxaspiro[3.5]nonan-7-yl)- 1H-indol-1- yl)cyclopropyl)-1,2,4- oxadiazol-5(4H)-one | 883.8 |
| 146 | 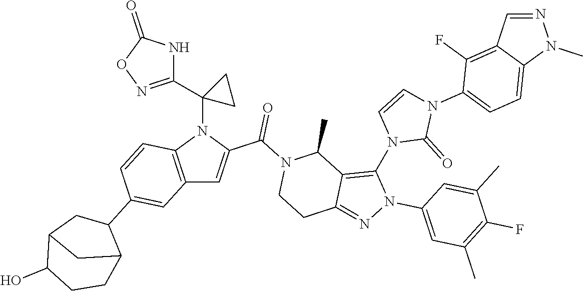 [SMILES: Cc1cc(-n2nc3c(c2-n2ccn(-c4ccc5c(cnn5C)c4F)c2=O)[C@H](C)N(C(=O)c2cc4cc(C5CC6CC5CCC6O)ccc4n2C2(c4noc(=O)[nH]4)CC2)CC3)cc(C)c1F] | 3-(1-(2-((S)-3-(3-(4-fluoro- 1-methyl-1H-indazol-5-yl)- 2-oxo-2,3-dihydro-1H- imidazol-1-yl)-2-(4-fluoro- 3,5-dimethylphenyl)-4- methyl-4,5,6,7-tetrahydro- 2H-pyrazolo[4,3- c]pyridine-5-carbonyl)-5- (2- hydroxybicyclo[3.2.1]octan- 6-yl)-1H-indol-1- yl)cyclopropyl)-1,2,4- oxadiazol-5(4H)-one | 881.6 |
| 147 | 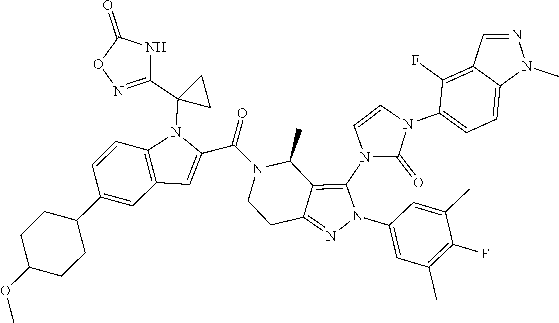 [SMILES: COC1CCC(c2ccc3c(c2)cc(C(=O)N2CCc4nn(-c5cc(C)c(F)c(C)c5)c(-n5ccn(-c6ccc7c(cnn7C)c6F)c5=O)c4[C@@H]2C)n3C2(c3noc(=O)[nH]3)CC2)CC1] | (S)-3-(1-(2-(3-(3-(4-fluoro- 1-methyl-1H-indazol-5-yl)- 2-oxo-2,3-dihydro-1H- imidazol-1-yl)-2-(4-fluoro- 3,5-dimethylphenyl)-4- methyl-4,5,6,7-tetrahydro- 2H-pyrazolo[4,3- c]pyridine-5-carbonyl)-5- (4-methoxycyclohexyl)-1H- indol-1-yl)cyclopropyl)- 1,2,4-oxadiazol-5(4H)-one | 869.8 |
| 216 Iso- mer A | 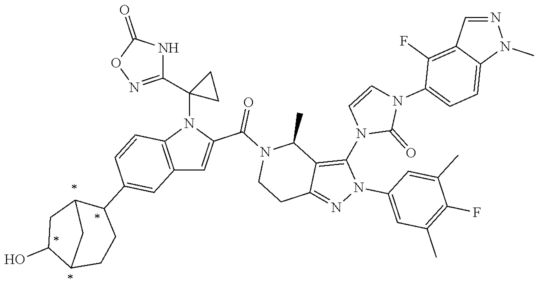 [SMILES: Cc1cc(-n2nc3c(c2-n2ccn(-c4ccc5c(cnn5C)c4F)c2=O)[C@H](C)N(C(=O)c2cc4cc(C5CCC6CC5CC6O)ccc4n2C2(c4noc(=O)[nH]4)CC2)CC3)cc(C)c1F] | See row below. | 881.7 |
| 216 | 3-(1-(2-((4S)-3-(3-(4-fluoro-1-methyl-1H-indazol-5-yl)-2-oxo-2,3-dihdyro-1H-imidazol-1-yl)- | | |
| Iso- | 2-(4-fluoro-3,5-dimethylphenyl)-4-methyl-4,5,6,7-tetrahydro-2H-pyrazolo[4,3-c]pyridine-5- | | |
| mer | carbonyl)-5-((1S,2S,5R,6S)-6-hydroxybicyclo[3.2.1]octan-2-yl)-1H-indol-1-yl)cyclopropyl)- | | |
| A | 1,2,4-oxadiazol-5(4H)-one or 3-(1-(2-((4S)-3-(3-(4-fluoro-1-methyl-1H-indazol-5-yl)-2-oxo- | | |
| | 2,3-dihydro-1H-imidazol-1-yl)-2-(4-fluoro-3,5-dimethylphenyl)-4-methyl-4,5,6,7-tetrahydro- | | |
| | 2H-pyrazolo[4,3-c]pyridine-5-carobnyl)-5-((1S,2S,5R,6R)-6-hydroxybicyclo[3.2.1]octan-2- | | |
| | yl)-1H-indol-1-yl)cyclopropyl)-1,2,4-oxadiazol-5(4H)-one or 3-(1-(2-((4S)-3-(3-(4-fluoro-1- | | |
| | methyl-1H-indazol-5-yl)-2-oxo-2,3-dihdyro-1H-imidazol-1-yl)-2-(4-fluoro-3,5- | | |
| | dimethylphenyl)-4-methyl-4,5,6,7-tetrahydro-2H-pyrazolo[4,3-c]pyridine-5-carbonyl)-5- | | |
| | ((1S,2R,5R,6S)-6-hydroxybicyclo[3.2.1]octan-2-yl)-1H-indol-1-yl)cyclopropyl)-1,2,4- | | |
| | oxadiazol-5(4H)-one or 3-(1-(2-((4S)-3-(3-(4-fluoro-1-methyl-1H-indaozl-5-yl)-2-oxo-2,3- | | |
| | dihydro-1H-imidazol-1-yl)-2-(4-fluoro-3,5-dimethylphenyl)-4-methyl-4,5,6,7-tetrahydro-2H- | | |
| | pyrazolo[4,3-c]pyridine-5-carbonyl)-5-((1S,2R,5R,6R)-6-hydroxybicyclo[3.2.1]octan-2-yl)- | | |
| | 1H-indol-1-yl)cyclopropyl)-1,2,4-oxadiazol-5(4H)-one | | |
| 216 Iso- mer B | 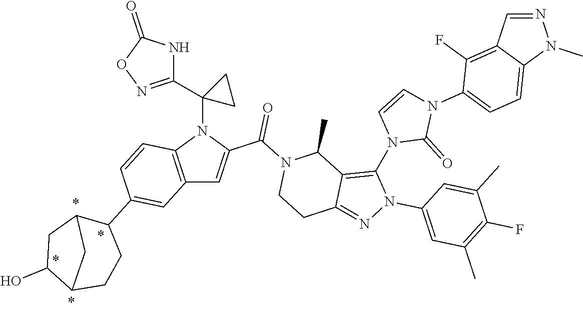 [SMILES: Cc1cc(-n2nc3c(c2-n2ccn(-c4ccc5c(cnn5C)c4F)c2=O)[C@H](C)N(C(=O)c2cc4cc(C5CCC6CC5CC6O)ccc4n2C2(c4noc(=O)[nH]4)CC2)CC3)cc(C)c1F] | See row below | 881.7 |
| 216 | 3-(1-(2-((4S)-3-(3-(4-fluoro-1-mehtyl-1H-indazol-5-yl)-2-oxo-2,3-dihdyro-1H-imidazol-1-yl)- | | |
| Iso- | 2-(4-fluoro-3,5-dimethylphenyl)-4-methyl-4,5,6,7-tetrahydro-2H-pyrazolo[4,3-c]pyridine-5- | | |
| mer | carbonyl)-5-((1S,2S,5R,6S)-6-hydroxybicyclo[3.2.1]octan-2-yl)-1H-indol-1-yl)cyclopropyl)- | | |
| B | 1,2,4-oxadiazol-5(4H)-one or 3-(1-(2-((4S)-3-(3-(4-fluoro-1-methyl-1H-indazol-5-yl)-2-oxo- | | |
| | 2,3-dihydro-1H-imidazol-1-yl)-2-(4-fluoro-3,5-dimethylphenyl)-4-methyl-4,5,6,7-tetrahydro- | | |
| | 2H-pyrazolo[4,3-c]pyridine-5-carbonyl)-5-((1S,2S,5R,6R)-6-hydroxybicyclo[3.2.1]octan-2- | | |
| | yl)-1H-indol-1-yl)cyclopropyl)-1,2,4-oxadiazol-5(4H)-one or 3-(1-(2-((4S)-3-(3-(4-fluoro-1- | | |
| | methyl-1H-indazol-5-yl)-2-oxo-2,3-dihydro-1H-imidazol-1-yl)-2-(4-fluoro-3,5- | | |
| | dimethylphenyl)-4-methyl-4,5,6,7-tetrahydro-2H-pyrazolo[4,3-c]pyridine-5-carbonyl)-5- | | |
| | ((1S,2R,5R,6S)-6-hydroxybicyclo[3.2.1]octan-2-yl)-1H-indol-1-yl)cyclopropyl)-1,2,4- | | |
| | oxadiazol-5(4H)-one or 3-(1-(2-((4S)-3-(3-(4-fluoro-1-methyl-1H-indazol-5-yl)-2-oxo-2,3- | | |
| | dihydro-1H-imidazol-1-yl)-2-(4-fluoro-3,5-dimethylphenyl)-4-methyl-4,5,6,7-tetrahydro-2H- | | |
| | pyrazolo[4,3-c]pyridine-5-carbonyl)-5-((1S,2R,5R,6R)-6-hydroxybicyclo[3.2.1]octan-2-yl)- | | |
| | 1H-indol-1-yl)cyclopropyl)-1,2,4-oxadiazol-5(4H)-one | | |
| 216 Iso- mer C | 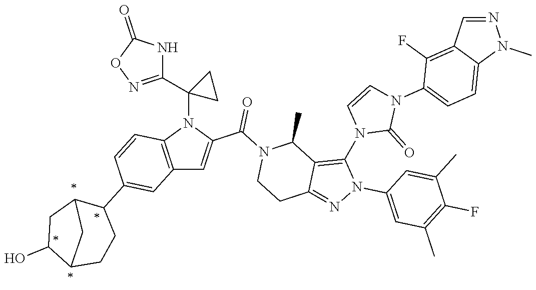 [SMILES: Cc1cc(-n2nc3c(c2-n2ccn(-c4ccc5c(cnn5C)c4F)c2=O)[C@H](C)N(C(=O)c2cc4cc(C5CCC6CC5CC6O)ccc4n2C2(c4noc(=O)[nH]4)CC2)CC3)cc(C)c1F] | see row below | 881.7 |
| 216 | 3-(1-(2-((4S)-3-(3-(4-fluoro-1-methyl-1H-indazol-5-yl)-2-oxo-2,3-dihydro-1H-imidazol-1-yl)- | | |
| Iso- | 2-(4-fluoro-3,5-dimethylphenyl)-4-methyl-4,5,6,7-tetrahydro-2H-pyrazolo[4,3-c]pyridine-5- | | |
| mer | carbonyl)-5-((1S,2S,5R,6S)-6-hydroxybicyclo[3.2.1]octan-2-y1)-1H-indol-1-yl)cyclopropyl)- | | |
| C | 1,2,4-oxadiazol-5(4H)-one or 3-(1-(2-((4S)-3-(3-(4-fluoro-1-methyl-1H-indazol-5-yl)-2-oxo- | | |
| | 2,3-dihydro-1H-imidazol-1-yl)-2-(4-fluoro-3,5-dimethylphenyl)-4-methyl-4,5,6,7-tetrahydro- | | |
| | 2H-pyrazolo[4,3-c]pyridine-5-carbonyl)-5-((1S,2S,5R,6R)-6-hydroxybicyclo[3.2.1]octan-2- | | |
| | yl)-1H-indol-1-yl)cyclopropyl)-1,2,4-oxadiazol-5(4H)-one or 3-(1-(2-((4S)-3-(3-(4-fluoro-1- | | |
| | methyl-1H-indazol-5-yl)-2-oxo-2,3-dihydro-1H-imidazol-1-yl)-2-(4-fluoro-3,5- | | |
| | dimethylphenyl)-4-methyl-4,5,6,7-tetrahydro-2H-pyrazolo[4,3-c]pyridine-5-carbonyl)-5- | | |
| | ((1S,2R,5R,6S)-6-hydroxybicyclo[3.2.1]octan-2-yl)-1H-indol-1-yl)cyclopropyl)-1,2,4- | | |
| | oxadiazol-5(4H)-one or 3-(1-(2-((4S)-3-(3-(4-fluoro-1-methyl-1H-indazol-5-yl)-2-oxo-2,3- | | |
| | dihydro-1H-imidazol-1-yl)-2-(4-fluoro-3,5-dimethylphenyl)-4-methyl-4,5,6,7-tetrahydro-2H- | | |
| | pyrazolo[4,3-c]pyridine-5-carbonyl)-5-((1S,2R,5R,6R)-6-hydroxybicyclo[3.2.1]octan-2-yl)- | | |
| | 1H-indol-1-yl)cyclopropyl)-1,2,4-oxadiazol-5(4H)-one | | |
| 216 Iso- mer D | 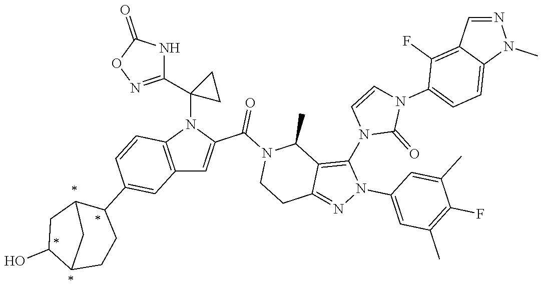 [SMILES: Cc1cc(-n2nc3c(c2-n2ccn(-c4ccc5c(cnn5C)c4F)c2=O)[C@H](C)N(C(=O)c2cc4cc(C5CCC6CC5CC6O)ccc4n2C2(c4noc(=O)[nH]4)CC2)CC3)cc(C)c1F] | See row below | 881.7 |
| 216 | 3-(1-(2-((4S)-3-(3-(4-fluoro-1-methyl-1H-indazol-5-yl)-2-oxo-2,3-dihydro-1H-imidazol-1-yl)- | | |
| Iso- | 2-(4-fluoro-3,5-dimethylphenyl)-4-methyl-4,5,6,7-tetrahydro-2H-pyrazolo[4,3-c]pyridine-5- | | |
| mer | carbonyl)-5-((1S,2S,5R,6S)-6-hydroxybicyclo[3.2.1]octan-2-yl)-1H-indol-1-yl)cyclopropyl)- | | |
| D | 1,2,4-oxadiazol-5(4H)-one or 3-(1-(2-((4S)-3-(3-(4-fluoro-1-methyl-1H-indazol-5-yl)-2-oxo- | | |
| | 2,3-dihydro-1H-imidazol-1-yl)-2-(4-fluoro-3,5-dimethylphenyl)-4-methyl-4,5,6,7-tetrahydro- | | |
| | 2H-pyrazolo[4,3-c]pyridine-5-carbonyl)-5-((1S,2S,5R,6R)-6-hydroxybicyclo[3.2.1 ]octan-2- | | |
| | yl)-1H-indol-1-yl)cyclopropyl)-1,2,4-oxadiazol-5(4H)-one or 3-(1-(2-((4S)-3-(3-(4-fluoro-1- | | |
| | methyl-1H-indazol-5-yl)-2-oxo-2,3-dihydro-1H-imidazol-1-yl)-2-(4-fluoro-3,5- | | |
| | dimethylphenyl)-4-methyl-4,5,6,7-tetrahydro-2H-pyrazolo[4,3-c]pyridine-5-carbonyl)-5- | | |
| | ((1S,2R,5R,6S)-6-hydroxybicyclo[3.2.1]octan-2-yl)-1H-indol-1-yl)cyclopropyl)-1,2,4- | | |
| | oxadiazol-5(4H)-one or 3-(1-(2-((4S)-3-(3-(4-fluoro-1-methyl-1H-indazol-5-yl)-2-oxo-2,3- | | |
| | dihydro-1H-imidazol-1-yl)-2-(4-fluoro-3,5-dimethylphenyl)-4-methyl-4,5,6,7-tetrahydro-2H- | | |
| | pyrazolo[4,3-c]pyridine-5-carbonyl)-5-((1S,2R,5R,6R)-6-hydroxybicyclo[3.2.1]octan-2-yl)- | | |
| | 1H-indol-1-yl)cyclopropyl)-1,2,4-oxadiazol-5(4H)-one | | |
| 217 | 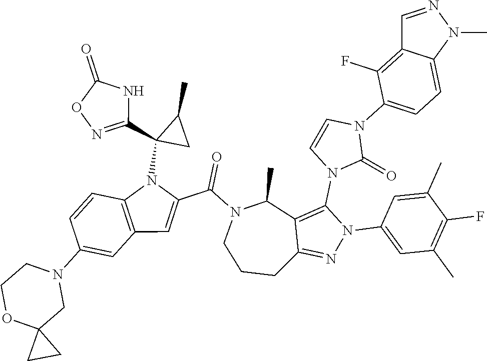 [SMILES: Cc1cc(-n2nc3c(c2-n2ccn(-c4ccc5c(cnn5C)c4F)c2=O)[C@H](C)N(C(=O)c2cc4cc(N5CCOC6(CC6)C5)ccc4n2[C@@]2(c4noc(=O)[nH]4)C[C@@H]2C)CCC3)cc(C)c1F] | 3-((1S,2S)-1-(2-((4S)-3-(3- (4-fluoro-1-methyl-1H- indazol-5-yl)-2-oxo-2,3- dihydro-1H-imidazol-1-yl)- 2-(4-fluoro-3,5- dimethylphenyl)-4-methyl- 2,4,5,6,7,8- hexahydropyrazolo[4,3- c]azepine-5-carbonyl)-5-(4- oxa-7-azaspiro[2.5]octan-7- yl)-1H-indol-1-yl)-2- methylcyclopropyl)-1,2,4- oxadiazol-5(4H)-one | 896.5 |
| 218 | 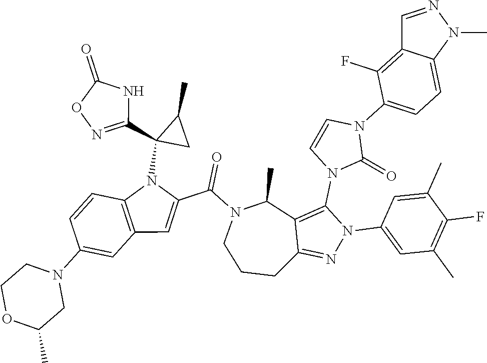 [SMILES: Cc1cc(-n2nc3c(c2-n2ccn(-c4ccc5c(cnn5C)c4F)c2=O)[C@H](C)N(C(=O)c2cc4cc(N5CCO[C@@H](C)C5)ccc4n2[C@@]2(c4noc(=O)[nH]4)C[C@@H]2C)CCC3)cc(C)c1F] | 3-((1S,2S)-1-(2-((4S)-3-(3- (4-fluoro-1-methyl-1H- indazol-5-yl)-2-oxo-2,3- dihydro-1H-imidazol-1-yl)- 2-(4-fluoro-3,5- dimethylphenyl)-4-methyl- 2,4,5,6,7,8- hexahydropyrazolo[4,3- c]azepine-5-carbonyl)-5- ((S)-2-methylmorpholino)- 1H-indol-1-yl)-2- methylcyclopropyl)-1,2,4- oxadiazol-5(4H)-one | 884.4 |
| 219 | 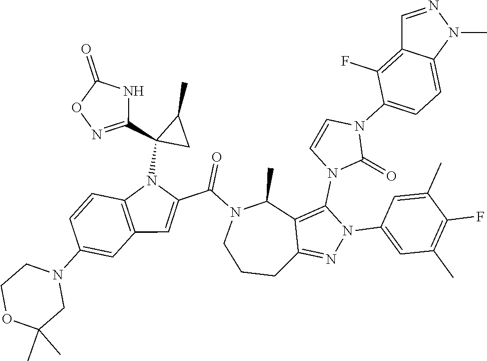 [SMILES: Cc1cc(-n2nc3c(c2-n2ccn(-c4ccc5c(cnn5C)c4F)c2=O)[C@H](C)N(C(=O)c2cc4cc(N5CCOC(C)(C)C5)ccc4n2[C@@]2(c4noc(=O)[nH]4)C[C@@H]2C)CCC3)cc(C)c1F] | 3-((1S,2S)-1-(5-(2,2- dimethylmorpholino)-2- ((4S)-3-(3-(4-fluoro-1- methyl-1H-indazol-5-yl)-2- oxo-2,3-dihydro-1H- imidazol-1-yl)-2-(4-fluoro- 3,5-dimethylphenyl)-4- methyl-2,4,5,6,7,8- hexahydropyrazolo[4,3- c]azepine-5-carbonyl)-1H- indol-1-yl)-2- methylcyclopropyl)-1,2,4- oxadiazol-5(4H)-one | 898.4 |
| 220 | 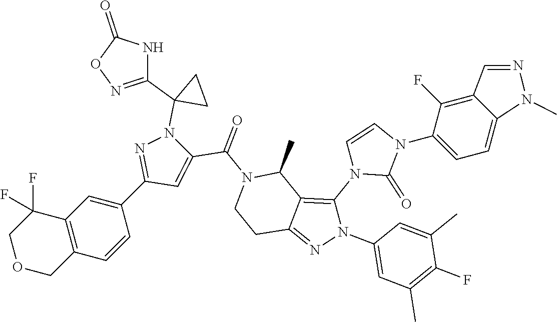 [SMILES: Cc1cc(-n2nc3c(c2-n2ccn(-c4ccc5c(cnn5C)c4F)c2=O)[C@H](C)N(C(=O)c2cc(-c4ccc5c(c4)C(F)(F)COC5)nn2C2(c4noc(=O)[nH]4)CC2)CC3)cc(C)c1F] | 3-(1-(3-(4,4- difluoroisochroman-6-yl)- 5-((4S)-3-(3-(4-fluoro-1- methyl-1H-indazol-5-yl)-2- oxo-2,3-dihydro-1H- imidazol-1-yl)-2-(4-fluoro- 3,5-dimethylphenyl)-4- methyl-4,5,6,7-tetrahydro- 2H-pyrazolo[4,3- c]pyridine-5-carbonyl)-1H- pyrazol-1-yl)cyclopropyl)- 1,2,4-oxadiazol-5(4H)-one | 876.3 |
| 221 | 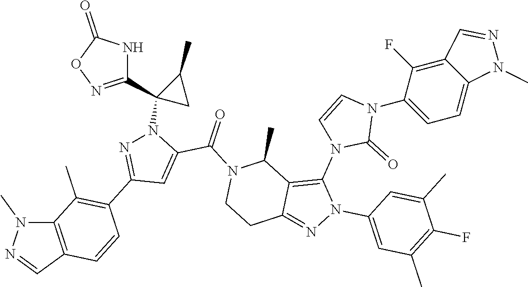 | 3-((1S,2S)-1-(3-(1,7- dimethyl-1H-indazol-6-yl)- 5-((4S)-3-(3-(4-fluoro-1- methyl-1H-indazol-5-yl)-2- oxo-2,3-dihydro-1H- imidazol-1-yl)-2-(4-fluoro- 3,5-dimethylphenyl)-4- methyl-4,5,6,7-tetrahydro- 2H-pyrazolo[4,3- c]pyridine-5-carbonyl)-1H- pyrazol-1-yl)-2- methylcyclopropyl)-1,2,4- oxadiazol-5(4H)-one | 864.5 |
| 353 | 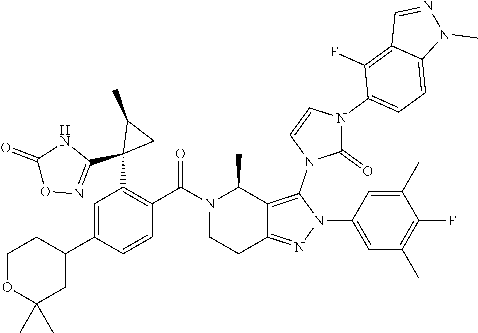 [SMILES: Cc1cc(-n2nc3c(c2-n2ccn(-c4ccc5c(cnn5C)c4F)c2=O)[C@H](C)N(C(=O)c2ccc(C4CCOC(C)(C)C4)cc2[C@@]2(c4noc(=O)[nH]4)C[C@@H]2C)CC3)cc(C)c1F] | 3-((1R,2S)-1-(5-((S)-2,2- dimethyltetrahydro-2H- pyran-4-yl)-2-((4S)-3-(3-(4- fluoro-1-methyl-1H- indazol-5-yl)-2-oxo-2,3- dihydro-1H-imidazol-1-yl)- 2-(4-fluoro-3,5- dimethylphenyl)-4-methyl- 4,5,6,7-tetrahydro-2H- pyrazolo[4,3-c]pyridine-5- carbonyl)phenyl)-2- methylcyclopropyl)-1,2,4- oxadiazol-5(4H)-one or 3- ((1R,2S)-1-(5-((S)-2,2- dimethyltetrahydro-2H- pyran-4-yl)-2-((4S)-3-(3-(4- fluoro-1-methyl-1H- indazol-5-yl)-2-oxo-2,3- dihydro-1H-imidazol-1-yl)- 2-(4-fluoro-3,5- dimethylphenyl)-4-methyl- | 844.0 |
| | | 4,5,6,7-tetrahydro-2H- | |
| | | pyrazolo[4,3-c]pyridine-5- | |
| | | carbonyl)phenyl)-2- | |
| | | methylcyclopropyl)-1,2,4- | |
| | | oxadiazol-5(4H)-one | |
| 364 | 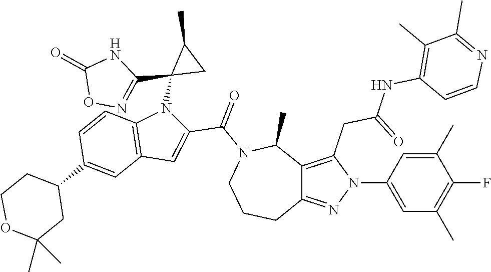 [SMILES: Cc1cc(-n2nc3c(c2CC(=O)Nc2ccnc(C)c2C)[C@H](C)N(C(=O)c2cc4cc([C@H]5CCOC(C)(C)C5)ccc4n2[C@@]2(c4noc(=O)[nH]4)C[C@@H]2C)CCC3)cc(C)c1F] | N-(2,3-dimethylpyridin-4- yl)-2-((S)-5-(5-((S)-2,2- dimethyltetrahydro-2H- pyran-4-yl)-1-((1S,2S)-2- methyl-1-(5-oxo-4,5- dihydro-1,2,4-oxadiazol-3- yl)cyclopropyl)-1H-indole- 2-carbonyl)-2-(4-fluoro- 3,5-dimethylphenyl)-4- methyl-2,4,5,6,7,8- hexahydropyrazolo[4,3- c]azepin-3-yl)acetamide | 829.4 |
| 365 | 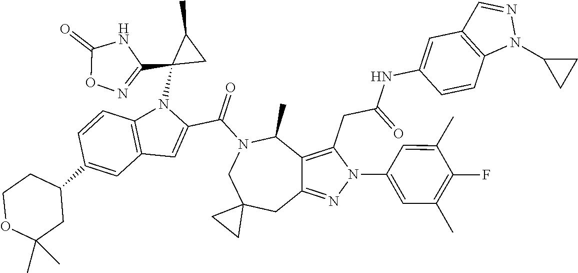 [SMILES: Cc1cc(-n2nc3c(c2CC(=O)Nc2ccc4c(cnn4C4CC4)c2)[C@H](C)N(C(=O)c2cc4cc([C@H]5CCOC(C)(C)C5)ccc4n2[C@@]2(c4noc(=O)[nH]4)C[C@@H]2C)CC2(CC2)C3)cc(C)c1F] | N-(1-cyclopropyl-1H- indazol-5-yl)-2-((S)-5′-(5- ((S)-2,2- dimethyltetrahydro-2H- pyran-4-yl)-1-((1S,2S)-2- methyl-1-(5-oxo-4,5- dihydro-1,2,4-oxadiazol-3- yl)cyclopropyl)-1H-indole- 2-carbonyl)-2′-(4-fluoro- 3,5-dimethylphenyl)-4′- methyl-2′,5′,6′,8′- tetrahydro-4′H- spiro[cyclopropane-1,7′- pyrazolo[4,3-c]azepin]-3′- yl)acetamide | 906.4 |
| 366 | 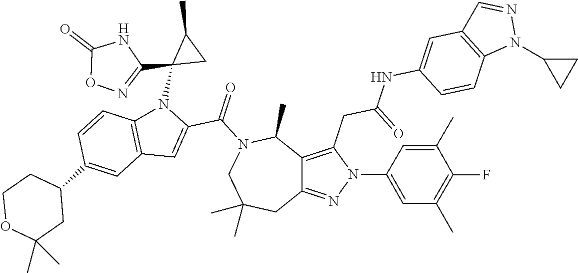 [SMILES: Cc1cc(-n2nc3c(c2CC(=O)Nc2ccc4c(cnn4C4CC4)c2)[C@H](C)N(C(=O)c2cc4cc([C@H]5CCOC(C)(C)C5)ccc4n2[C@@]2(c4noc(=O)[nH]4)C[C@@H]2C)CC(C)(C)C3)cc(C)c1F] | N-(1-cyclopropyl-1H- indazol-5-yl)-2-((S)-5-(5- ((S)-2,2- dimethyltetrahydro-2H- pyran-4-yl)-1-((1S,2S)-2- methyl-1-(5-oxo-4,5- dihydro-1,2,4-oxadiazol-3- yl)cyclopropyl)-1H-indole- 2-carbonyl)-2-(4-fluoro- 3,5-dimethylphenyl)-4,7,7- trimethyl-2,4,5,6,7,8- hexahydropyrazolo[4,3- c]azepin-3-yl)acetamide | 908.5 |
| 367 | 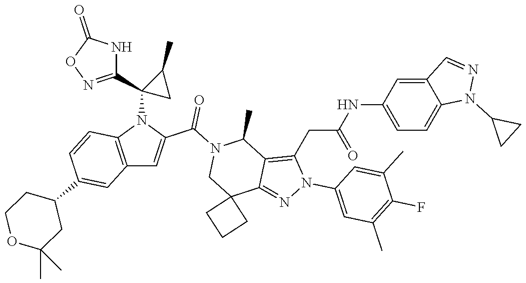 [SMILES: Cc1cc(-n2nc3c(c2CC(=O)Nc2ccc4c(cnn4C4CC4)c2)[C@H](C)N(C(=O)c2cc4cc([C@H]5CCOC(C)(C)C5)ccc4n2[C@@]2(c4noc(=O)[nH]4)C[C@@H]2C)CC32CCC2)cc(C)c1F] | N-(1-cyclopropyl-1H- indazol-5-yl)-2-((S)-5′-(5- ((S)-2,2- dimethyltetrahydro-2H- pyran-4-yl)-1-((1S,2S)-2- methyl-1-(5-oxo-4,5- dihydro-1,2,4-oxadiazol-3- yl)cyclopropyl)-1H-indole- 2-carbonyl)-2′-(4-fluoro- 3,5-dimethylphenyl)-4′- methyl-2′,4′,5′,6′- tetrahydrospiro[cyclobutane- 1,7′-pyrazolo[4,3- c]pyridin]-3′-yl)acetamide | 907.0 |
| 368 | 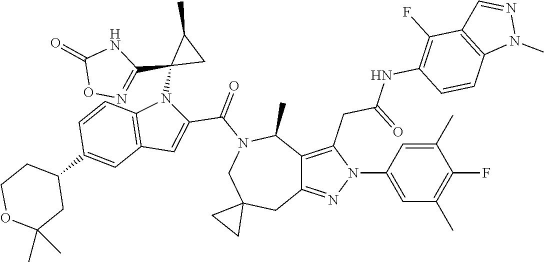 [SMILES: Cc1cc(-n2nc3c(c2CC(=O)Nc2ccc4c(cnn4C)c2F)[C@H](C)N(C(=O)c2cc4cc([C@H]5CCOC(C)(C)C5)ccc4n2[C@@]2(c4noc(=O)[nH]4)C[C@@H]2C)CC2(CC2)C3)cc(C)c1F] | 2-((S)-5′-(5-((S)-2,2- dimethyltetrahydro-2H- pyran-4-yl)-1-((1S,2S)-2- methyl-1-(5-oxo-4,5- dihydro-1,2,4-oxadiazol-3- yl)cyclopropyl)-1H-indole- 2-carbonyl)-2′-(4-fluoro- 3,5-dimethylphenyl)-4′- methyl-2′,5′,6′,8′- tetrahydro-4′H- spiro[cyclopropane-1,7'- pyrazolo[4,3-c]azepin]-3′- yl)-N-(4-fluoro-1-methyl- 1H-indazol-5-yl)acetamide | 898.6 |
| 369 | 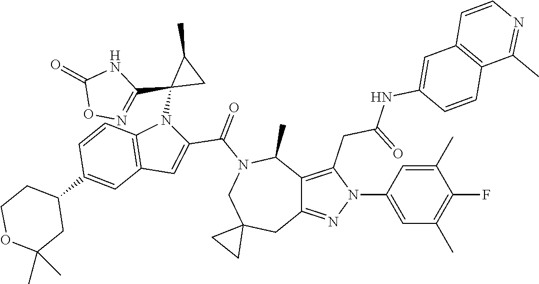 [SMILES: Cc1cc(-n2nc3c(c2CC(=O)Nc2ccc4c(C)nccc4c2)[C@H](C)N(C(=O)c2cc4cc([C@H]5CCOC(C)(C)C5)ccc4n2[C@@]2(c4noc(=O)[nH]4)C[C@@H]2C)CC2(CC2)C3)cc(C)c1F] | 2-((S)-5′-(5-((S)-2,2- dimethyltetrahydro-2H- pyran-4-yl)-1-((1S,2S)-2- methyl-1-(5-oxo-4,5- dihydro-1,2,4-oxadiazol-3- yl)cyclopropyl)-1H-indole- 2-carbonyl)-2′-(4-fluoro- 3,5-dimethylphenyl)-4′- methyl-2′,5′,6′,8′- tetrahydro-4′H- spiro[cyclopropane-1,7′- pyrazolo[4,3-c]azepin]-3′- yl)-N-(1-methylisoquinolin- 6-yl)acetamide | 892.4 |
| 370 | 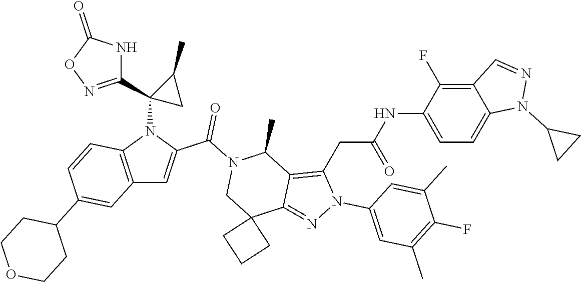 [SMILES: Cc1cc(-n2nc3c(c2CC(=O)Nc2ccc4c(cnn4C4CC4)c2F)[C@H](C)N(C(=O)c2cc4cc(C5CCOCC5)ccc4n2[C@@]2(c4noc(=O)[nH]4)C[C@@H]2C)CC32CCC2)cc(C)c1F] | N-(1-cyclopropyl-4-fluoro- 1H-indazol-5-yl)-2-((S)-2′- (4-fluoro-3,5- dimethylphenyl)-4′-methyl- 5′-(1-((1S,2S)-2-methyl-1- (5-oxo-4,5-dihydro-1,2,4- oxadiazol-3- yl)cyclopropyl)-5- (tetrahydro-2H-pyran-4-yl)- 1H-indole-2-carbonyl)- 2′,4′,5′,6′- tetrahydrospiro[cyclobutane- 1,7′-pyrazolo[4,3- c]pyridin]-3′-yl)acetamide | 896.7 |
| 371 | 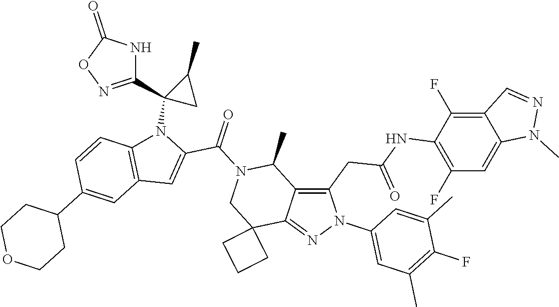 [SMILES: Cc1cc(-n2nc3c(c2CC(=O)Nc2c(F)cc4c(cnn4C)c2F)[C@H](C)N(C(=O)c2cc4cc(C5CCOCC5)ccc4n2[C@@]2(c4noc(=O)[nH]4)C[C@@H]2C)CC32CCC2)cc(C)c1F] | N-(4,6-difluoro-1-methyl- 1H-indazol-5-yl)-2-((S)-2′- (4-fluoro-3,5- dimethylphenyl)-4′-methyl- 5′-(1-((1S,2S)-2-methyl-1- (5-oxo-4,5-dihydro-1,2,4- oxadiazol-3- yl)cyclopropyl)-5- (tetrahydro-2H-pyran-4-yl)- 1H-indole-2-carbonyl)- 2′,4′,5′,6′- tetrahydrospiro[cyclobutane- 1,7′-pyrazolo[4,3- c]pyridin]-3′-yl)acetamide | 888.9 |
| 372 | 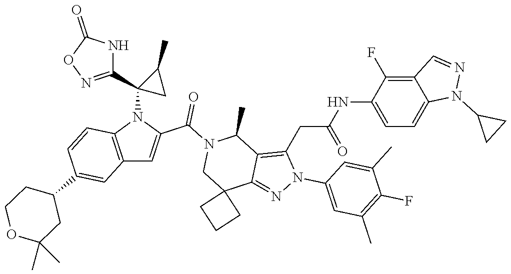 [SMILES: Cc1cc(-n2nc3c(c2CC(=O)Nc2ccc4c(cnn4C4CC4)c2F)[C@H](C)N(C(=O)c2cc4cc([C@H]5CCOC(C)(C)C5)ccc4n2[C@@]2(c4noc(=O)[nH]4)C[C@@H]2C)CC32CCC2)cc(C)c1F] | N-(1-cyclopropyl-4-fluoro- 1H-indazol-5-y1)-2-((S)-5′- (5-((S)-2,2- dimethyltetrahydro-2H- pyran-4-y1)-1-((1S,2S)-2- methyl-1-(5-oxo-4,5- dihydro-1,2,4-oxadiazol-3- yl)cyclopropyl)-1H-indole- 2-carbonyl)-2′-(4-fluoro- 3,5-dimethylphenyl)-4'- methyl-2′,4′,5′,6′- tetrahydrospiro[cyclobutane- 1,7′-pyrazolo[4,3- c]pyridin]-3'-yl)acetamide | 924.8 |
| 373 | 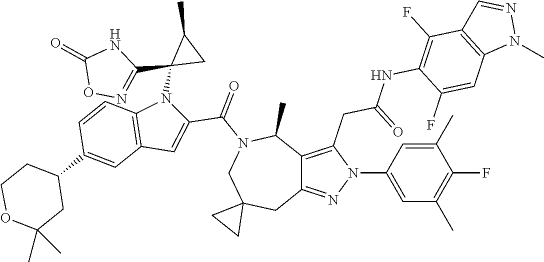 [SMILES: Cc1cc(-n2nc3c(c2CC(=O)Nc2c(F)cc4c(cnn4C)c2F)[C@H](C)N(C(=O)c2cc4cc([C@H]5CCOC(C)(C)C5)ccc4n2[C@@]2(c4noc(=O)[nH]4)C[C@@H]2C)CC2(CC2)C3)cc(C)c1F] | N-(4,6-difluoro-1-methyl- 1H-indazol-5-yl)-2-((S)-5′- (5-((S)-2,2- dimethyltetrahydro-2H- pyran-4-yl)-1-((1S,2S)-2- methyl-1-(5-oxo-4,5- dihydro-1,2,4-oxadiazol-3- yl)cyclopropyl)-1H-indole- 2-carbonyl)-2′-(4-fluoro- 3,5-dimethylphenyl)-4′- methyl-2′,5′,6′,8′- tetrahydro-4'H- spiro[cyclopropane-1,7′- pyrazolo[4,3-c]azepin]-3′- yl)acetamide | 916.7 |
| 374 | 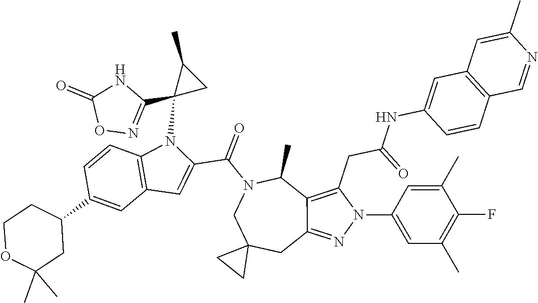 [SMILES: Cc1cc2cc(NC(=O)Cc3c4c(nn3-c3cc(C)c(F)c(C)c3)CC3(CC3)CN(C(=O)c3cc5cc([C@H]6CCOC(C)(C)C6)ccc5n3[C@@]3(c5noc(=O)[nH]5)C[C@@H]3C)[C@H]4C)ccc2cn1] | 2-((S)-5′-(5-((S)-2,2- dimethyltetrahydro-2H- pyran-4-y1)-1-((1S,2S)-2- methyl-1-(5-oxo-4,5- dihydro-1,2,4-oxadiazol-3- yl)cyclopropyl)-1H-indole- 2-carbonyl)-2′-(4-fluoro- 3,5-dimethylphenyl)-4'- methyl-2′,5′,6′,8′- tetrahydro-4′H- spiro[cyclopropane-1,7′- pyrazolo[4,3-c]azepin]-3′- yl)-N-(3-methylisoquinolin- 6-yl)acetamide | |
| 375 | 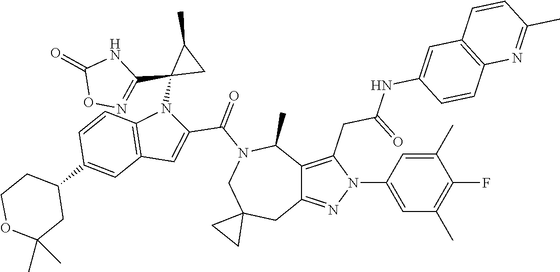 [SMILES: Cc1ccc2cc(NC(=O)Cc3c4c(nn3-c3cc(C)c(F)c(C)c3)CC3(CC3)CN(C(=O)c3cc5cc([C@H]6CCOC(C)(C)C6)ccc5n3[C@@]3(c5noc(=O)[nH]5)C[C@@H]3C)[C@H]4C)ccc2n1] | 2-((S)-5′-(5-((S)-2,2- dimethyltetrahydro-2H- pyran-4-yl)-1-((1S,2S)-2- methyl-1-(5-oxo-4,5- dihydro-1,2,4-oxadiazol-3- yl)cyclopropyl)-1H-indole- 2-carbonyl)-2′-(4-fluoro- 3,5-dimethylphenyl)-4′- methyl-2′,5′,6′,8′- tetrahydro-4′H- spiro[cyclopropane-1,7′- pyrazolo[4,3-c]azepin]-3′- yl)-N-(2-methylquinolin-6- yl)acetamide | 891.4 |
| 376 | 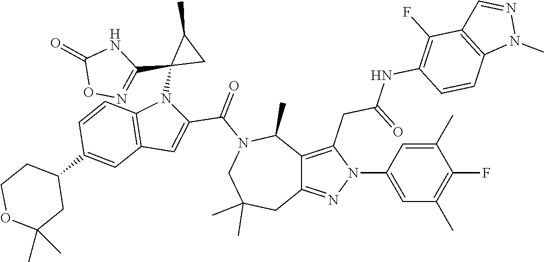 [SMILES: Cc1cc(-n2nc3c(c2CC(=O)Nc2ccc4c(cnn4C)c2F)[C@H](C)N(C(=O)c2cc4cc([C@H]5CCOC(C)(C)C5)ccc4n2[C@@]2(c4noc(=O)[nH]4)C[C@@H]2C)CC(C)(C)C3)cc(C)c1F] | 2-((S)-5-(5-((S)-2,2- dimethyltetrahydro-2H- pyran-4-yl)-1-((1S,2S)-2- methyl-1-(5-oxo-4,5- dihydro-1,2,4-oxadiazol-3- yl)cyclopropyl)-1H-indole- 2-carbonyl)-2-(4-fluoro- 3,5-dimethylphenyl)-4,7,7- trimethyl-2,4,5,6,7,8- hexahydropyrazolo[4,3- c]azepin-3-yl)-N-(4-fluoro- 1-methyl-1H-indazol-5- yl)acetamide | 900.4 |
| 417 | 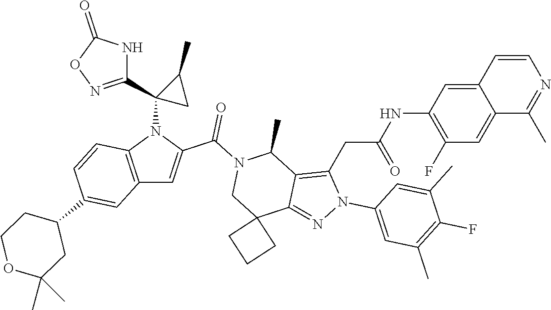 [SMILES: Cc1cc(-n2nc3c(c2CC(=O)Nc2cc4ccnc(C)c4cc2F)[C@H](C)N(C(=O)c2cc4cc([C@H]5CCOC(C)(C)C5)ccc4n2[C@@]2(c4noc(=O)[nH]4)C[C@@H]2C)CC32CCC2)cc(C)c1F] | N-(1-cyclopropyl-4-fluoro- 1H-indazol-5-y1)-2-((S)-5′- (5-((S)-2,2- dimethyltetrahydro-2H- pyran-4-yl)-1-((1S,2S)-2- methyl-1-(5-oxo-4,5- dihydro-1,2,4-oxadiazol-3- yl)cyclopropyl)-1H-indole- 2-carbonyl)-2′-(4-fluoro- 3,5-dimethylphenyl)-4′- methyl-2′,4′,5′,6′- tetrahydrospiro[cyclobutane- 1,7′-pyrazolo[4,3- c]pyridin]-3′-yl)acetamide | 909.4 |
| 418 | 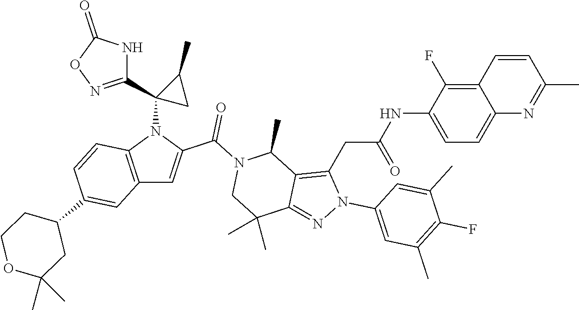 [SMILES: Cc1ccc2c(F)c(NC(=O)Cc3c4c(nn3-c3cc(C)c(F)c(C)c3)C(C)(C)CN(C(=O)c3cc5cc([C@H]6CCOC(C)(C)C6)ccc5n3[C@@]3(c5noc(=O)[nH]5)C[C@@H]3C)[C@H]4C)ccc2n1] | 2-((S)-5-(5-((S)-2,2- dimethyltetrahydro-2H- pyran-4-yl)-1-((1S,2S)-2- methyl-1-(5-oxo-4,5- dihydro-1,2,4-oxadiazol-3- yl)cyclopropyl)-1H-indole- 2-carbonyl)-2-(4-fluoro- 3,5-dimethylphenyl)-4,7,7- trimethyl-4,5,6,7- tetrahydro-2H- pyrazolo[4,3-c]pyridin-3- yl)-N-(5-fluoro-2- methylquinolin-6- yl)acetamide | 897.4 |
| 419 | 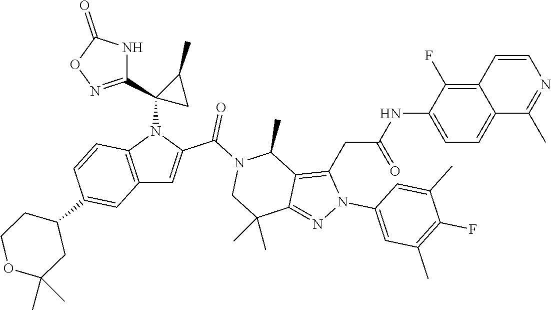 [SMILES: Cc1cc(-n2nc3c(c2CC(=O)Nc2ccc4c(C)nccc4c2F)[C@H](C)N(C(=O)c2cc4cc([C@H]5CCOC(C)(C)C5)ccc4n2[C@@]2(c4noc(=O)[nH]4)C[C@@H]2C)CC3(C)C)cc(C)c1F] | 2-((S)-5-(5-((S)-2,2- dimethyltetrahydro-2H- pyran-4-yl)-1-((1S,2S)-2- methyl-1-(5-oxo-4,5- dihydro-1,2,4-oxadiazol-3- yl)cyclopropyl)-1H-indole- 2-carbonyl)-2-(4-fluoro- 3,5-dimethylphenyl)-4,7,7- trimethyl-4,5,6,7- tetrahydro-2H- pyrazolo[4,3-c]pyridin-3- yl)-N-(5-fluoro-1- methylisoquinolin-6- yl)acetamide | 897.4 |
| 420 | 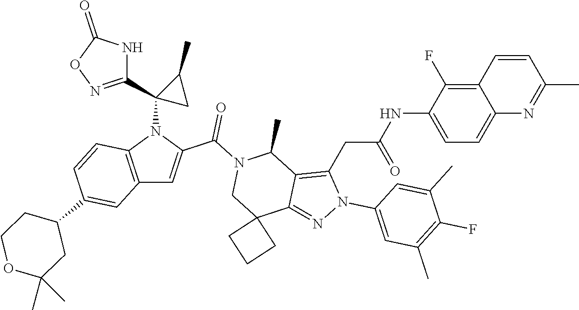 [SMILES: Cc1ccc2c(F)c(NC(=O)Cc3c4c(nn3-c3cc(C)c(F)c(C)c3)C3(CCC3)CN(C(=O)c3cc5cc([C@H]6CCOC(C)(C)C6)ccc5n3[C@@]3(c5noc(=O)[nH]5)C[C@@H]3C)[C@H]4C)ccc2n1] | 2-((S)-5′-(5-((S)-2,2- dimethyltetrahydro-2H- pyran-4-yl)-1-((1S,2S)-2- methyl-1-(5-oxo-4,5- dihydro-1,2,4-oxadiazol-3- yl)cyclopropyl)-1H-indole- 2-carbonyl)-2′-(4-fluoro- 3,5-dimethylphenyl)-4′- methyl-2′,4′,5′,6′- tetrahydrospiro[cyclobutane- 1,7′-pyrazolo[4,3- c]pyridin]-3′-y1)-N-(5- fluoro-2-methylquinolin-6- yl)acetamide | 909.4 |
| 421 | 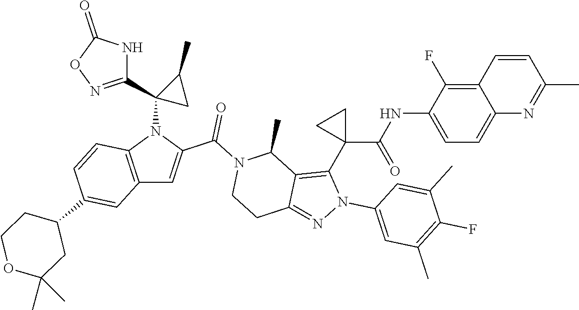 [SMILES: Cc1ccc2c(F)c(NC(=O)C3(c4c5c(nn4-c4cc(C)c(F)c(C)c4)CCN(C(=O)c4cc6cc([C@H]7CCOC(C)(C)C7)ccc6n4[C@@]4(c6noc(=O)[nH]6)C[C@@H]4C)[C@H]5C)CC3)ccc2n1] | 1-((S)-5-(5-((S)-2,2- dimethyltetrahydro-2H- pyran-4-yl)-1-((1S,2S)-2- methyl-1-(5-oxo-4,5- dihydro-1,2,4-oxadiazol-3- yl)cyclopropyl)-1H-indole- 2-carbonyl)-2-(4-fluoro- 3,5-dimethylphenyl)-4- methyl-4,5,6,7-tetrahydro- 2H-pyrazolo[4,3-c]pyridin- 3-yl)-N-(5-fluoro-2- methylquinolin-6- yl)cyclopropane-1- carboxamide | 896.1 |

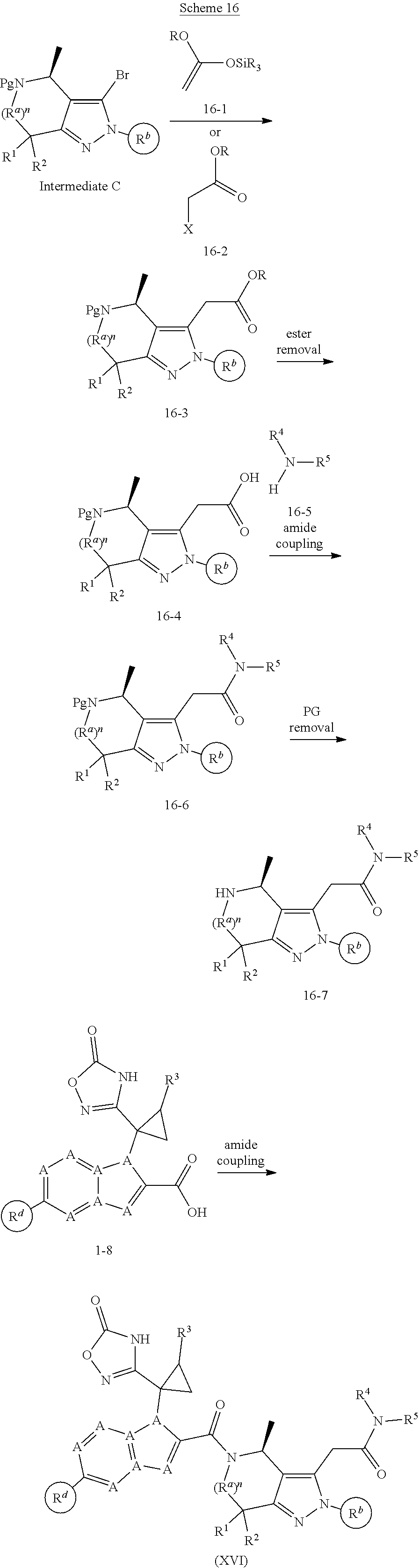

Compounds of formula (XVI) are prepared from intermediate C by coupling with silyl enol ethers (16-1) or metal enolates such as zinc enolates (16-2, depicted arbitrarily as one possible tautomer) via transition metal catalyzed cross-coupling conditions (e.g., Pd—or Cu-catalysis) to provide 16-3. Removal of the ester (e.g., basic aqueous conditions) of 16-3 furnishes carboxylic acid 16-4. Subsequently, 16-4 is coupled with amines 16-5 to provide amide 16-6. Removal of the protecting group (e.g., acidic conditions) of 16-6 provides amine 16-7 that is then coupled with carboxylic acid 1-8 to provide compounds of formula (XVI).

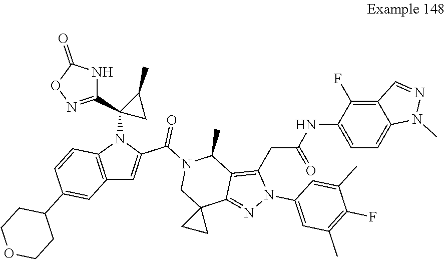 [SMILES: Cc1cc(-n2nc3c(c2CC(=O)Nc2ccc4c(cnn4C)c2F)[C@H](C)N(C(=O)c2cc4cc(C5CCOCC5)ccc4n2[C@@]2(c4noc(=O)[nH]4)C[C@@H]2C)CC32CC2)cc(C)c1F]

### N-(4-fluoro-1-methyl-1H-indazol-5-yl)-2-((S)-2′-(4-fluoro-3,5-dimethylphenyl)-4′-methyl-5′-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-2′,4′,5′,6′-tetrahydrospiro[cyclopropane-1,7′-pyrazolo[4,3-c]pyridin]-3′-yl)acetamide (scheme 16)

### Step 1: tert-butyl (S)-2′-(4-fluoro-3,5-dimethylphenyl)-3′-(2-methoxy-2-oxoethyl)-4′-methyl-2′,4′-dihydrospiro[cyclopropane-1,7′-pyrazolo[4,3-c]pyridine]-5′(6′H)-carboxylate

A mixture of tert-butyl (S)-3′-bromo-2′-(4-fluoro-3,5-dimethylphenyl)-4′-methyl-2′,4′-dihydrospiro[cyclopropane-1,7′-pyrazolo[4,3-c]pyridine]-5′(6′H)-carboxylate (100 mg, 0.215 mmol), lithium fluoride (33.5 mg, 1.29 mmol) and bis(tri-t-butylphosphine)palladium(0) (22.0 mg, 0.043 mmol) was placed under argon. To the vial were then added DMF (1.08 mL) and tert-butyl((1-methoxyvinyl)oxy)dimethylsilane (122 mg, 142 μL, 0.646 mmol). The mixture was stirred at 100° C. overnight. At this point, the reaction mixture was treated with additional lithium fluoride and bis(tri-t-butylphosphine)palladium(0). After placing under argon, to the mixture was added additional tert-butyl((1-methoxyvinyl)oxy)dimethylsilane. The reaction mixture was stirred at 100° C. for 20 min. The mixture was then cooled down and purified directly via silica gel column chromatography (0-80% EtOAc/hexanes) to afford the title compound as an oil. MS (ESI) m/z: 458.4 [M+H]+.

### Step 2: (S)-2-(5′-(tert-butoxycarbonyl)-2′-(4-fluoro-3,5-dimethylphenyl)-4′-methyl-2′,4′,5′,6′-tetrahydrospiro[cyclopropane-1,7′-pyrazolo[4,3-c]pyridin]-3′-yl)acetic acid

A mixture of tert-butyl (S)-2′-(4-fluoro-3,5-dimethylphenyl)-3′-(2-methoxy-2-oxoethyl)-4′-methyl-2′,4′-dihydrospiro[cyclopropane-1,7′-pyrazolo[4,3-c]pyridine]-5′(6′H)-carboxylate (68.0 mg, 0.149 mmol) and lithium hydroxide monohydrate (9.4 mg, 0.223 mmol) in methanol (0.74 mL) was stirred at rt overnight. The mixture was then treated with additional lithium hydroxide monohydrate (24.9 mg, 0.594 mmol) and water (0.20 mL), and stirred at 50° C. for 30 min. The reaction mixture was then concentrated and partitioned between 1 M aqueous HCl and DCM. The aqueous layer was separated and back-extracted with DCM twice. The combined organic layers were dried over MgSO₄, filtered, and concentrated to afford the title compound as a solid, which was used without further purification. MS (ESI) m/z: 444.4 [M+H]+.

### Step 3: tert-butyl (S)-3′-(2-((4-fluoro-1-methyl-1H-indazol-5-yl)amino)-2-oxoethyl)-2′-(4-fluoro-3,5-dimethylphenyl)-4′-methyl-2′,4′-dihydrospiro[cyclopropane-1,7′-pyrazolo[4,3-c]pyridine]-5′(6′H)-carboxylate

A mixture of (S)-2-(5′-(tert-butoxycarbonyl)-2′-(4-fluoro-3,5-dimethylphenyl)-4′-methyl-2′,4′,5′,6′-tetrahydrospiro[cyclopropane-1,7′-pyrazolo[4,3-c]pyridin]-3′-yl)acetic acid (30.0 mg, 0.068 mmol), 4-fluoro-1-methyl-1H-indazole-5-amine (16.8 mg, 0.101 mmol) and HATU (38.6 mg, 0.101 mmol) in DMF (0.48 mL) was treated with DIPEA (43.7 mg, 59 μL, 0.338 mmol) and stirred at rt for 10 min. The reaction mixture was then directly purified via silica gel column chromatography (0-100% EtOAc/hexanes) to afford the title compound as a solid. MS (ESI) m/z: 591.6 [M+H]+.

### Step 4: (S)—N-(4-fluoro-1-methyl-1H-indazol-5-yl)-2-(2′-(4-fluoro-3,5-dimethylphenyl)-4′-methyl-2′,4′,5,6′-tetrahydrospiro[cyclopropane-1,7′-pyrazolo[4,3-c]pyridin]-3′-yl)acetamide

A mixture of tert-butyl (S)-3′-(2-((4-fluoro-1-methyl-1H-indazol-5-yl)amino)-2-oxoethyl)-2′-(4-fluoro-3,5-dimethylphenyl)-4′-methyl-2′,4′-dihydrospiro[cyclopropane-1,7′-pyrazolo[4,3-c]pyridine]-5′(6′H)-carboxylate (15.0 mg, 0.025 mmol) and 4 M HCl/dioxane (0.13 mL, 0.51 mmol) was stirred at rt for 30 min. The reaction mixture was then concentrated to afford the title compound as a solid, which was used further without purification.

### Step 5: N-(4-fluoro-1-methyl-1H-indazol-5-yl)-2-((S)-2′-(4-fluoro-3,5-dimethylphenyl)-4′-methyl-5′-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-2′,4′,5′,6′-tetrahydrospiro[cyclopropane-1,7′-pyrazolo[4,3-c]pyridin]-3′-yl)acetamide

To crude (S)—N-(4-fluoro-1-methyl-1H-indazol-5-yl)-2-(2′-(4-fluoro-3,5-dimethylphenyl)-4′-methyl-2′,4′,5′,6′-tetrahydrospiro[cyclopropane-1,7′-pyrazolo[4,3-c]pyridin]-3′-yl)acetamide (0.025 mmol) from Step 4 was added 1-((1S,2S)-2-methyl-1-(5-oxo-4,5-dihydro-1,2,4-oxadiazol-3yl)cyclopropyl)-5-(tetrahydro-2H-pyran-4-yl)-1H-indole-2-carboxylic acid (14.6 mg, 0.038 mmol), HATU (14.5 mg, 0.038 mmol) and DMF (0.25 mL). The mixture was then treated with DIPEA (23.0 mg, 31 μL, 0.178 mmol) and stirred at rt overnight. The reaction mixture was then purified via prep-HPLC (60-90% MeCN/water+0.1% formic acid modifier) to afford the title compound as a solid. ¹H NMR (500 MHz, CD₃CN) δ 8.30-7.94 (m, 1H), 7.93-7.41 (m, 3H), 7.35-7.14 (m, 3H), 7.07 (d, J=6.3 Hz, 1H), 7.00-6.68 (m, 2H), 6.33-5.43 (m, 1H), 4.25-3.94 (m, 6H), 3.94-3.74 (m, 2H), 3.70-3.36 (m, 3H), 2.92-2.79 (m, 1H), 2.30-2.23 (m, 6H), 1.82-1.50 (m, 9H), 1.49-1.23 (m, 3H), 1.19-1.07 (m, 1H), 1.07-0.86 (m, 3H), 0.83-0.44 (in, 1H). MS (ESI) m/z: 856.8 [M+H]+. The following examples in table 13 were prepared according to scheme 16 using the procedures outlined in the synthesis of example 148.

TABLE 13

| | | | MS |
|---|---|---|---|
| Ex. | Structure | Name | (M + 1) |
| 149 | 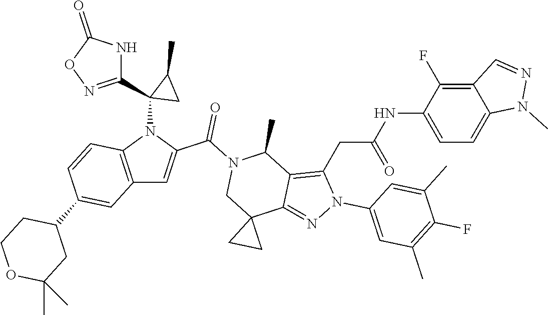 | 2-((S)-5′-(5-((S)-2,2- dimethyltetrahydro-2H- pyran-4-yl)-1-((1S,2S)-2- methyl-1-(5-oxo-4,5-dihydro- 1,2,4-oxadiazol-3- yl)cyclopropyl)-1H-indole-2- carbonyl)-2′-(4-fluoro-3,5- dimethylphenyl)-4′-methyl- 2′,4′,5′,6′- tetrahydrospiro[cyclopropane- 1,7′-pyrazolo[4,3-c]pyridin]- 3′-yl)-N-(4-fluoro-1-methyl- 1H-indazol-5-yl)acetamide | 884.8 |
| 150 | 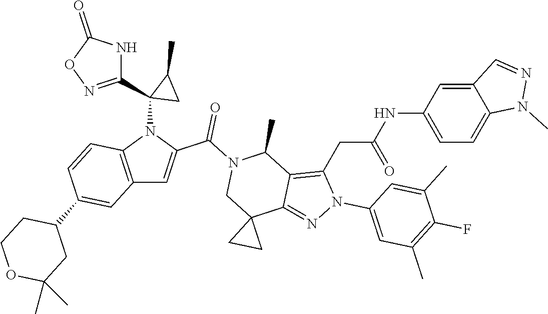 | 2-((S)-5′-(5-((S)-2,2- dimethyltetrahydro-2H- pyran-4-yl)-1-((1S,2S)-2- methyl-1-(5-oxo-4,5-dihydro- 1,2,4-oxadiazol-3- yl)cyclopropyl)-1H-indole-2- carbonyl)-2′-(4-fluoro-3,5- dimethylphenyl)-4'-methyl- 2′,4′,5′,6′- tetrahydrospiro[cyclopropane- 1,7′-pyrazolo[4,3-c]pyridin]- 3′-yl)-N-(1-methyl-1H- indazol-5-yl)acetamide | 866.8 |
| 151 | 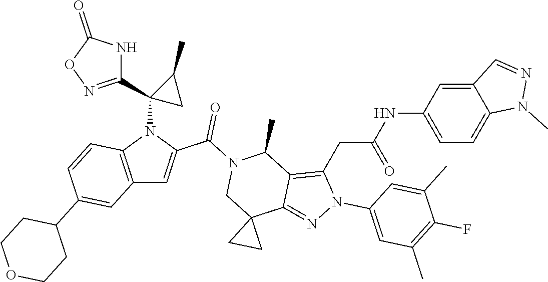 [SMILES: Cc1cc(-n2nc3c(c2CC(=O)Nc2ccc4c(cnn4C)c2)[C@H](C)N(C(=O)c2cc4cc(C5CCOCC5)ccc4n2[C@@]2(c4noc(=O)[nH]4)C[C@@H]2C)CC32CC2)cc(C)c1F] | 2-((S)-2'-(4-fluoro-3,5- dimethylphenyl)-4′-methyl-5′- (1-((1S,2S)-2-methyl-1-(5- oxo-4,5-dihydro-1,2,4- oxadiazol-3-yl)cyclopropyl)- 5-(tetrahydro-2H-pyran-4-yl)- 1H-indole-2-carbonyl)- 2′,4′,5′,6′- tetrahydrospiro[cyclopropane- 1,7′-pyrazolo[4,3-c]pyridin]- 3′-yl)-N-(1-methyl-1H- indazol-5-yl)acetamide | 838.8 |
| 152 | 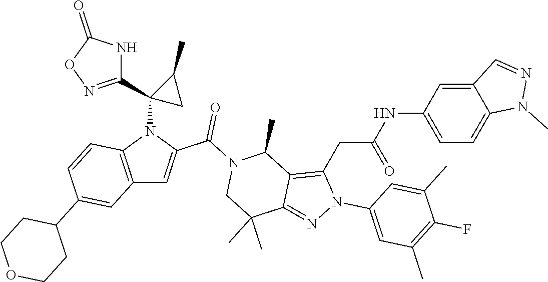 [SMILES: Cc1cc(-n2nc3c(c2CC(=O)Nc2ccc4c(cnn4C)c2)[C@H](C)N(C(=O)c2cc4cc(C5CCOCC5)ccc4n2[C@@]2(c4noc(=O)[nH]4)C[C@@H]2C)CC3(C)C)cc(C)c1F] | 2-((S)-2-(4-fluoro-3,5- dimethylphenyl)-4,7,7- trimethyl-5-(1-((1S,2S)-2- methyl-1-(5-oxo-4,5-dihydro- 1,2,4-oxadiazol-3- yl)cyclopropyl)-5- (tetrahydro-2H-pyran-4-yl)- 1H-indole-2-carbonyl)- 4,5,6,7-tetrahydro-2H- pyrazolo[4,3-c]pyridin-3-yl)- N-(1-methyl-1H-indazol-5- yl)acetamide | 840.6 |
| 153 | 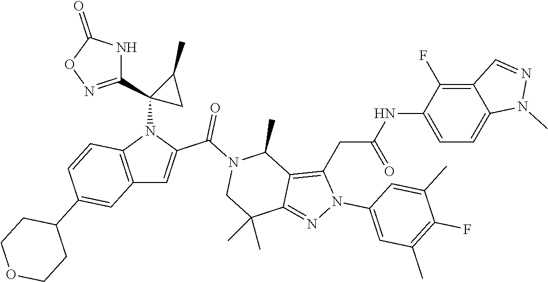 [SMILES: Cc1cc(-n2nc3c(c2CC(=O)Nc2ccc4c(cnn4C)c2F)[C@H](C)N(C(=O)c2cc4cc(C5CCOCC5)ccc4n2[C@@]2(c4noc(=O)[nH]4)C[C@@H]2C)CC3(C)C)cc(C)c1F] | N-(4-fluoro-1-methyl-1H- indazol-5-yl)-2-((S)-2-(4- fluoro-3,5-dimethylphenyl)- 4,7,7-trimethyl-5-(1-((1S,2S)- 2-methyl-1-(5-oxo-4,5- dihydro-1,2,4-oxadiazol-3- yl)cyclopropyl)-5- (tetrahydro-2H-pyran-4-yl)- 1H-indole-2-carbonyl)- 4,5,6,7-tetrahydro-2H- pyrazolo[4,3-c]pyridin-3- yl)acetamide | 858.6 |
| 154 | 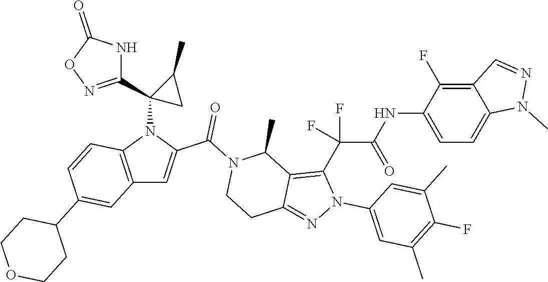 [SMILES: Cc1cc(-n2nc3c(c2C(F)(F)C(=O)Nc2ccc4c(cnn4C)c2F)[C@H](C)N(C(=O)c2cc4cc(C5CCOCC5)ccc4n2[C@@]2(c4noc(=O)[nH]4)C[C@@H]2C)CC3)cc(C)c1F] | 2,2-difluoro-N-(4-fluoro-1- methyl-1H-indazol-5-yl)-2- ((S)-2-(4-fluoro-3,5- dimethylphenyl)-4-methyl-5- (1-((1S,2S)-2-methyl-1-(5- oxo-4,5-dihydro-1,2,4- oxadiazol-3-yl)cyclopropyl)- 5-(tetrahydro-2H-pyran-4-yl)- 1H-indole-2-carbonyl)- 4,5,6,7-tetrahydro-2H- pyrazolo[4,3-c]pyridin-3- yl)acetamide | 866.6 |
| 222 | 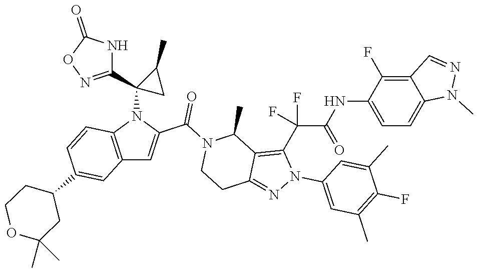 [SMILES: Cc1cc(-n2nc3c(c2C(F)(F)C(=O)Nc2ccc4c(cnn4C)c2F)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | 2-((S)-5-(5-((S)-2,2- dimethyltetrahydro-2H- pyran-4-yl)-1-((1S,2S)-2- methyl-1-(5-oxo-4,5-dihydro- 1,2,4-oxadiazol-3- yl)cyclopropyl)-1H-indole-2- carbonyl)-2-(4-fluoro-3,5- dimethylphenyl)-4-methyl- 4,5,6,7-tetrahydro-2H- pyrazolo[4,3-c]pyridin-3-yl)- 2,2-difluoro-N-(4-fluoro-1- methyl-1H-indazol-5- yl)acetamide | 894.6 |
| 223 | 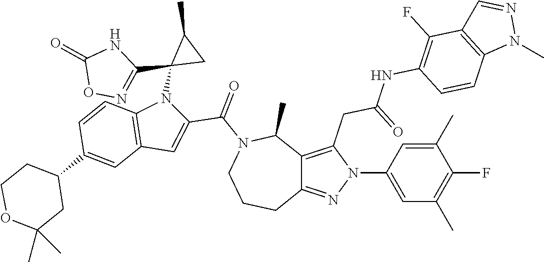 [SMILES: Cc1cc(-n2nc3c(c2CC(=O)Nc2ccc4c(cnn4C)c2F)[C@H](C)N(C(=O)c2cc4cc([C@H]5CCOC(C)(C)C5)ccc4n2[C@@]2(c4noc(=O)[nH]4)C[C@@H]2C)CCC3)cc(C)c1F] | 2-[(4S)-5-[5-[(4S)-2,2- dimethyltetrahydropyran-4- yl]-1-[(1S,2S)-2-methyl-1-(5- oxo-4H-1,2,4-oxadiazol-3- yl)cyclopropyl]indole-2- carbonyl]-2-(4-fluoro-3,5- dimethyl-phenyl)-4-methyl- 4,6,7,8- tetrahydropyrazolo[4,3- c]azepin-1-ium-3-yl]-N-(4- fluoro-1-methyl-indazol-5- yl)acetamide formate | 872.4 |
| 224 | 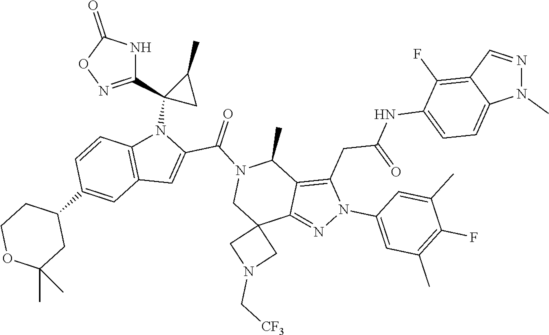 [SMILES: Cc1cc(-n2nc3c(c2CC(=O)Nc2ccc4c(cnn4C)c2F)[C@H](C)N(C(=O)c2cc4cc([C@H]5CCOC(C)(C)C5)ccc4n2[C@@]2(c4noc(=O)[nH]4)C[C@@H]2C)CC32CN(CC(F)(F)F)C2)cc(C)c1F] | 2-((S)-5′-(5-((S)-2,2- dimethyltetrahydro-2H- pyran-4-yl)-1-((1S,2S)-2- methyl-1-(5-oxo-4,5-dihydro- 1,2,4-oxadiazol-3- yl)cyclopropyl)-1H-indole-2- carbonyl)-2′-(4-fluoro-3- methylphenyl)-4′-methyl-1- (2,2,2-trifluoroethyl)- 2′,4′,5′,6′- tetrahydrospiro[azetidine- 3,7′-pyrazolo[4,3-c]pyridin]- 3′-yl)-N-(4-fluoro-1-methyl- 1H-indazol-5-yl)acetamide | 981.4 |
| 250 | 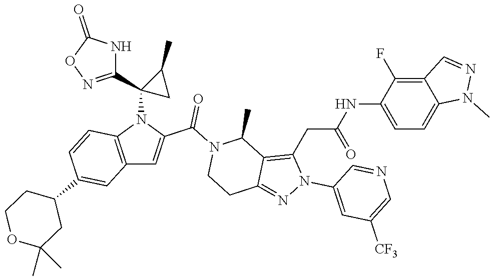 [SMILES: C[C@H]1c2c(nn(-c3cncc(C(F)(F)F)c3)c2CC(=O)Nc2ccc3c(cnn3C)c2F)CCN1C(=O)c1cc2cc([C@H]3CCOC(C)(C)C3)ccc2n1[C@@]1(c2noc(=O)[nH]2)C[C@@H]1C] | 2-((S)-5-(5-((S)-2,2- dimethyltetrahydro-2H- pyran-4-yl)-1-((1S,2S)-2- methyl-1-(5-oxo-4,5-dihydro- 1,2,4-oxadiazol-3- yl)cyclopropyl)-1H-indole-2- carbonyl)-4-methyl-2-(5- (trifluoromethyl)pyridin-3- yl)-4,5,6,7-tetrahydro-2H- pyrazolo[4,3-c]pyridin-3-yl)- N-(4-fluoro-1-methyl-1H- indazol-5-yl)acetamide | 881.2 |
| 338 | 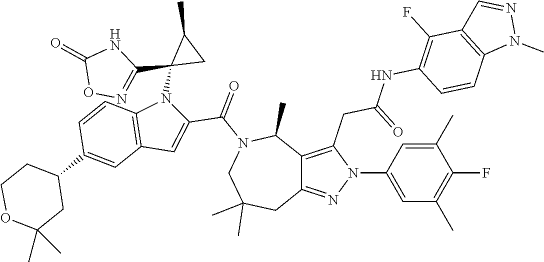 [SMILES: Cc1cc(-n2nc3c(c2CC(=O)Nc2ccc4c(cnn4C)c2F)[C@H](C)N(C(=O)c2cc4cc([C@H]5CCOC(C)(C)C5)ccc4n2[C@@]2(c4noc(=O)[nH]4)C[C@@H]2C)CC(C)(C)C3)cc(C)c1F] | 2-((S)-5-(5-((S)-2,2- dimethyltetrahydro-2H- pyran-4-yl)-1-((1S,2S)-2- methyl-1-(5-oxo-4,5-dihydro- 1,2,4-oxadiazol-3- yl)cyclopropyl)-1H-indole-2- carbonyl)-2-(4-fluoro-3,5- dimethylphenyl)-4,7,7- trimethyl-2,4,5,6,7,8- hexahydropyrazolo[4,3- c]azepin-3-yl)-N-(4-fluoro-1- methyl-1H-indazol-5- yl)acetamide | 900.4 |

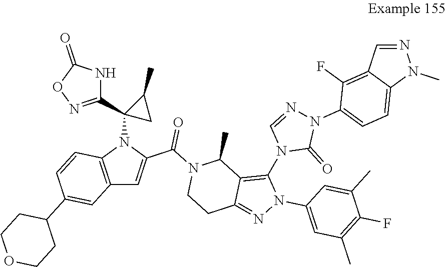 [SMILES: Cc1cc(-n2nc3c(c2-n2cnn(-c4ccc5c(cnn5C)c4F)c2=O)[C@H](C)N(C(=O)c2cc4cc(C5CCOCC5)ccc4n2[C@@]2(c4noc(=O)[nH]4)C[C@@H]2C)CC3)cc(C)c1F]

### 3-((1S,2S)-1-(2-((S)-3-(1-(4-fluoro-1-methyl-1H-indazol-5-yl)-5-oxo-1,5-dihydro-4H-1,2,4-triazol-4-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one

### Step 1: tert-butyl (S)-2-(4-fluoro-3,5-dimethylphenyl)-4-methyl-3-(5-oxo-1,5-dihydro-4H-1,2,4-triazol-4-yl)-2,4,6,7-tetrahydro-5H-pyrazolo[4,3-c]pyridine-5-carboxylate

To a solution of 4-nitrophenyl carbonochloridate (1.40 g, 6.94 mmol) in DCM (30 mL) at 0° C. was added tert-butyl (S)-3-amino-2-(4-fluoro-3,5-dimethylphenyl)-4-methyl-2,4,6,7-tetrahydro-5H-pyrazolo[4,3-c]pyridine-5-carboxylate (2 g, 5.34 mmol) and pyridine (0.734 mL, 9.08 mmol). The reaction mixture was stirred at 20° C. for 3 h, then N₂H₄·H₂O (3.51 g, 59.6 mmol) was added. The resulting mixture was stirred at 20° C. for 1 h, then poured into water (30 mL) and extracted with DCM (3×30 mL). The combined organic layers were washed with brine (20 mL), dried over Na₂SO₄ and concentrated under reduced pressure to provide tert-butyl (S)-2-(4-fluoro-3,5-dimethylphenyl)-3-(hydrazinecarboxamido)-4-methyl-2,4,6,7-tetrahydro-5H-pyrazolo[4,3-c]pyridine-5-carboxylate which was used in the next step directly without further purification. To a solution of tert-butyl (S)-2-(4-fluoro-3,5-dimethylphenyl)-3-(hydrazinecarboxamido)-4-methyl-2,4,6,7-tetrahydro-5H-pyrazolo[4,3-c]pyridine-5-carboxylate (500 mg, 1.16 mmol), triethoxymethane (223 mg, 1.50 mmol) in MeOH (10 mL) was added 4-methylbenzenesulfonic acid (20.0 mg, 0.116 mmol). The reaction mixture was stirred at 90° C. by microwave heating for 1 h. Then, the reaction mixture was purified by prep-HPLC (55% to 75% MeCN/H2O+0.1% TFA gradient) to provide the title compound. MS (ESI) m/z: 443.1 [M+H]+.

### Step 2: tert-butyl (S)-3-(1-(4-fluoro-1-methyl-1H-indazol-5-yl)-5-oxo-1,5-dihydro-4H-1,2,4-triazol-4-yl)-2-(4-fluoro-3,5-dimethylphenyl)-4-methyl-2,4,6,7-tetrahydro-5H-pyrazolo[4,3-c]pyridine-5-carboxylate

To a solution of tert-butyl (S)-2-(4-fluoro-3,5-dimethylphenyl)-4-methyl-3-(5-oxo-1,5-dihydro-4H-1,2,4-triazol-4-yl)-2,4,6,7-tetrahydro-5H-pyrazolo[4,3-c]pyridine-5-carboxylate (300 mg, 0.678 mmol), 5-bromo-4-fluoro-1-methyl-1H-indazole (311 mg, 1.36 mmol), (1S,2S)—N1,N2-dimethylcyclohexane-1,2-diamine (48.2 mg, 0.339 mmol) and potassium phosphate (576 mg, 2.71 mmol) in dioxane (10 mL) was added copper(I) iodide (25.8 mg, 0.136 mmol). The reaction mixture was stirred at 100° C. under an atmosphere of argon for 16 h. Then, the reaction mixture was cooled to rt, then the reaction poured into water (20 mL) and extracted with ethyl acetate (3×30 mL). The combined organic layers were washed with brine (3×20 mL), dried over Na₂SO₄ and concentrated under reduced pressure. The crude residue was purified by silica gel chromatography (0 to 50% EtOAc/Pet.ether) to provide the title compound. MS (ESI) m/z: 591.2 [M+H]+.

### Steps 3 and 4: 3-((1S,2S)-1-(2-((S)-3-(1-(4-fluoro-1-methyl-1H-indazol-5-yl)-5-oxo-1,5-dihydro-4H-1,2,4-triazol-4-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one

These steps were performed in a similar fashion as described for example 1 to provide the title compound as a TFA salt. ¹H NMR (400 MHz, MeOD) δ=8.25-8.17 (m, 1H), 8.14-7.90 (m, 1H), 7.60-7.37 (m, 4H), 7.30-7.07 (m, 3H), 6.94-6.83 (m, 1H), 5.91-5.36 (m, 1H), 4.62-4.46 (m, 1H), 4.14 (s, 5H), 3.73-3.50 (m, 3H), 3.26 (br s, 4H), 2.34-2.27 (m, 6H), 1.87-1.71 (m, 6H), 1.62-1.51 (m, 3H), 1.32-1.08 (m, 3H). MS (ESI) m/z: 856.3 [M+H]+.

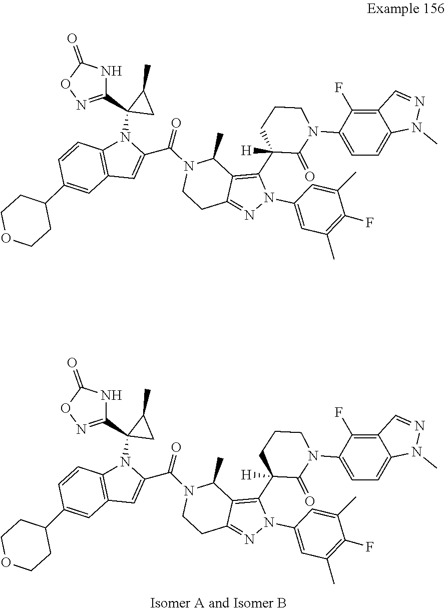 [SMILES: Cc1cc(-n2nc3c(c2[C@@H]2CCCN(c4ccc5c(cnn5C)c4F)C2=O)[C@H](C)N(C(=O)c2cc4cc(C5CCOCC5)ccc4n2[C@@]2(c4noc(=O)[nH]4)C[C@@H]2C)CC3)cc(C)c1F ; Cc1cc(-n2nc3c(c2[C@H]2CCCN(c4ccc5c(cnn5C)c4F)C2=O)[C@H](C)N(C(=O)c2cc4cc(C5CCOCC5)ccc4n2[C@@]2(c4noc(=O)[nH]4)C[C@@H]2C)CC3)cc(C)c1F]

### 3-((1S,2S)-1-(2-((S)-3-((S)-1-(4-fluoro-1-methyl-1H-indazol-5-yl)-2-oxopiperidin-3-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one and 3-((1S,2S)-1-(2-((S)-3-((R)-1-(4-fluoro-1-methyl-1H-indazol-5-yl)-2-oxopiperidin-3-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one

### Step 1: tert-butyl (S)-3-((S)-1-(4-fluoro-1-methyl-1H-indazol-5-yl)-2-oxopiperidin-3-yl)-2-(4-fluoro-3,5-dimethylphenyl)-4-methyl-2,4,6,7-tetrahydro-5H-pyrazolo[4,3-c]pyridine-5-carboxylate and tert-butyl (S)-3-((R)-1-(4-fluoro-1-methyl-1H-indazol-5-yl)-2-oxopiperidin-3-yl)-2-(4-fluoro-3,5-dimethylphenyl)-4-methyl-2,4,6,7-tetrahydro-5H-pyrazolo[4,3-c]pyridine-5-carboxylate

A solution of tert-butyl (S)-3-(1-(4-fluoro-1-methyl-1H-indazol-5-yl)-2-oxo-1,2,5,6-tetrahydropyridin-3-yl)-2-(4-fluoro-3,5-dimethylphenyl)-4-methyl-2,4,6,7-tetrahydro-5H-pyrazolo[4,3-c]pyridine-5-carboxylate (270 mg, 0.45 mmol) in EtOAc (5.0 mL) was purged with N₂. Platinum(IV) oxide (31 mg, 140 mmol) was added. The atmosphere was replaced with H₂ (via 3 purge-pump cycles) and stirred at rt for 16 h under an atmosphere of H₂. Then, the H₂ atmosphere was replaced (with an inert atmosphere) and the mixture was filtered through Celite® and washed with DCM (3×10 mL). The filtrate was concentrated under reduced pressure. The crude mixture of the two stereoisomers was purified by chiral SFC (column name: i-Cellulose-5 methanol/isocratic) to afford isomer A (faster eluting): MS (ESI) m/z 605.3 [M+H]+. Isomer B (slower eluting): MS (ESI) m/z 605.4 [M+H]+.

### Step 2: (S)-1-(4-fluoro-1-methyl-1H-indazol-5-yl)-3-((S)-2-(4-fluoro-3,5-dimethylphenyl)-4-methyl-4,5,6,7-tetrahydro-2H-pyrazolo[4,3-c]pyridin-3-yl)piperidin-2-one and (R)-1-(4-fluoro-1-methyl-1H-indazol-5-yl)-3-((S)-2-(4-fluoro-3,5-dimethylphenyl)-4-methyl-4,5,6,7-tetrahydro-2H-pyrazolo[4,3-c]pyridin-3-yl)piperidin-2-one

This step was performed for each isomer separately to provide the title compounds. A mixture of tert-butyl (S)-3-((S)-1-(4-fluoro-1-methyl-1H-indazol-5-yl)-2-oxopiperidin-3-yl)-2-(4-fluoro-3,5-dimethylphenyl)-4-methyl-2,4,6,7-tetrahydro-5H-pyrazolo[4,3-c]pyridine-5-carboxylate and tert-butyl (S)-3-((R)-1-(4-fluoro-1-methyl-1H-indazol-5-yl)-2-oxopiperidin-3-yl)-2-(4-fluoro-3,5-dimethylphenyl)-4-methyl-2,4,6,7-tetrahydro-5H-pyrazolo[4,3-c]pyridine-5-carboxylate (50 mg, 0.083 mmol) and 4M HCl/dioxane (0.21 mL, 0.83 mmol) was stirred at 25° C. for 1 h. Then, Et₂O (10 mL) was added to the mixture. The resulting solid precipitate was filtered and washed with Et₂O (2×5 mL) to provide the title compound. Data for isomer A: MS (ESI) m/z 505.3 [M+H]+. Data for isomer B: MS (ESI) m/z 505.3 [M+H]+.

### Step 3: 3-((1S,2S)-1-(2-((S)-3-((S)-1-(4-fluoro-1-methyl-1H-indazol-5-yl)-2-oxopiperidin-3-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one and 3-((1S,2S)-1-(2-((S)-3-((R)-1-(4-fluoro-1-methyl-1H-indazol-5-yl)-2-oxopiperidin-3-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one

This step was performed in a similar fashion as described for example 1 for each isomer separately to provide the title compounds. Data for Isomer A. ¹H NMR (400 MHz, methanol-d4): δH 8.10 (1H, s), 7.42-7.54 (3H, m), 7.23-7.29 (3H, m), 7.12 (1H, d, J=6.2 Hz), 6.86-6.94 (1H, m), 5.90-6.0 (0.5H, m), 5.42-5.50 (0.3H, m), 4.78-4.80 (0.3H, m), 4.40-4.43 (0.5H, m), 4.05-4.09 (4H, m), 4.03 (2H, s), 3.89 (1H, br s), 3.53-3.65 (4H, m), 2.85-3.16 (3H, m), 2.14-2.33 (9H, m), 1.46-1.89 (11H, m), 1.27 (2H, d, J=6.0 Hz), 1.04 (1H, d, J=6.1 Hz). MS (ESI) m/z 869.9 [M+H]+. Data for Isomer B. ¹H NMR (400 MHz, methanol-d4): δH 8.11 (0.6H, s), 7.96 (0.4H, s), 7.45-7.52 (2H, m), 7.38 (1H, d, J=8.7 Hz), 7.27 (1H, d, J=9.4 Hz), 7.13-7.18 (2H, m), 6.87 (0.5H, s), 6.82 (0.5H, s), 5.82-5.85 (0.5H, m), 5.42-5.44 (0.5H, m), 4.72-4.75 (0.5H, m), 4.40-4.43 (0.5H, m), 4.09 (3H, s), 4.04 (3H, s), 3.87-3.89 (1H, m), 3.74 (1H, br.s), 3.54-3.64 (4H, m), 3.44-3.46 (1H, m), 2.86-3.12 (4H, m), 2.31-2.35 (6H, m), 2.04 (2H, br.s), 1.74-1.88 (8H, m), 1.57-1.65 (1H, m), 1.19-1.41 (3H, m), 0.96-0.98 (1H, m). MS (ESI) m/z 869.9 [M+H]+.

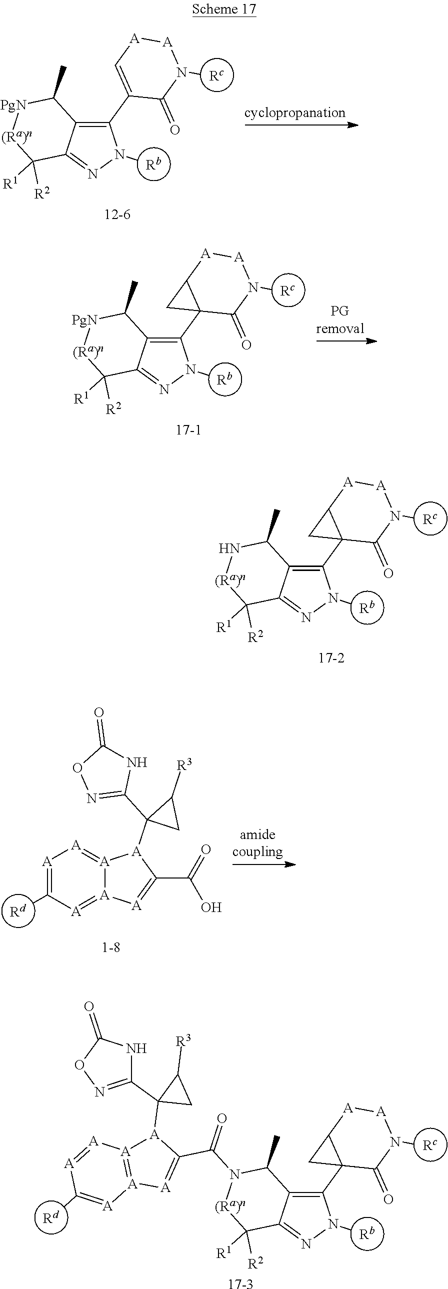

Compounds of formula 17-3 are prepared from intermediate 12-6 by cyclopropanation, followed by removal of the protecting group (e.g., acidic conditions) and afterwards coupling with carboxylic acid 1-8.

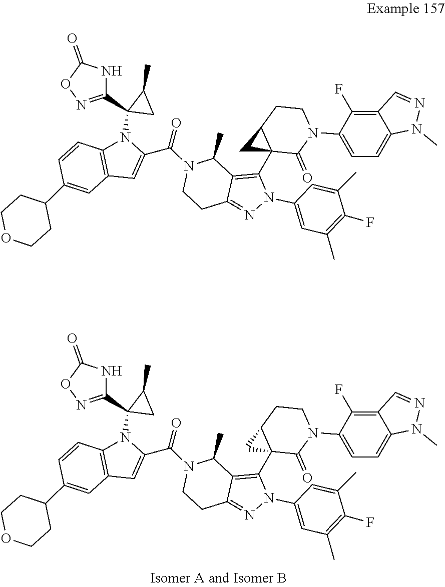 [SMILES: Cc1cc(-n2nc3c(c2[C@]24C[C@H]2CCN(c2ccc5c(cnn5C)c2F)C4=O)[C@H](C)N(C(=O)c2cc4cc(C5CCOCC5)ccc4n2[C@@]2(c4noc(=O)[nH]4)C[C@@H]2C)CC3)cc(C)c1F ; Cc1cc(-n2nc3c(c2[C@@]24C[C@@H]2CCN(c2ccc5c(cnn5C)c2F)C4=O)[C@H](C)N(C(=O)c2cc4cc(C5CCOCC5)ccc4n2[C@@]2(c4noc(=O)[nH]4)C[C@@H]2C)CC3)cc(C)c1F]

### 3-((1S,2S)-1-(2-((S)-3-((1S,6S)-3-(4-fluoro-1-methyl-1H-indazol-5-yl)-2-oxo-3-azabicyclo[4.1.0]heptan-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one and 3-((1S,2S)-1-(2-((S)-3-((1R,6R)-3-(4-fluoro-1-methyl-1H-indazol-5-yl)-2-oxo-3-azabicyclo[4.1.0]heptan-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one

### Step 1: tert-butyl (S)-3-((1S,6S)-3-(4-fluoro-1-methyl-1H-indazol-5-yl)-2-oxo-3-azabicyclo[4.1.0]heptan-1-yl)-2-(4-fluoro-3,5-dimethylphenyl)-4-methyl-2,4,6,7-tetrahydro-5H-pyrazolo[4,3-c]pyridine-5-carboxylate and tert-butyl (S)-3-((1R,6R)-3-(4-fluoro-1-methyl-1H-indazol-5-yl)-2-oxo-3-azabicyclo[4.1.0]heptan-1-yl)-2-(4-fluoro-3,5-dimethylphenyl)-4-methyl-2,4,6,7-tetrahydro-5H-pyrazolo[4,3-c]pyridine-5-carboxylate

To a suspension of sodium hydride (16 mg, 0.65 mmol) (60% in oil dispersion) in dry DMSO (4.0 mL) was added trimethyl(oxo)sulfonium iodide (140 mg, 0.65 mmol) at rt. The mixture was stirred for 30 min, then a solution of tert-butyl (S)-3-(1-(4-fluoro-1-methyl-1H-indazol-5-yl)-2-oxo-1,2,5,6-tetrahydropyridin-3-yl)-2-(4-fluoro-3,5-dimethylphenyl)-4-methyl-2,4,6,7-tetrahydro-5H-pyrazolo[4,3-c]pyridine-5-carboxylate (130 mg, 0.22 mmol) in DMSO (1.0 mL) was added slowly (drop-wise addition). The resulting mixture was stirred at 23° C. for 2 h, then poured into ice-water (10 mL), extracted with CH₂Cl₂. The organic layer was washed with brine, dried on MgSO₄ and concentrated under reduced pressure to afford the title compound. MS (ESI) m/z 616.7 (pk 1), 616.7 (pk 2) [M+H]+.

### Step 2: (1S,6S)-3-(4-fluoro-1-methyl-1H-indazol-5-yl)-1-((S)-2-(4-fluoro-3,5-dimethylphenyl)-4-methyl-4,5,6,7-tetrahydro-2H-pyrazolo[4,3-c]pyridin-3-yl)-3-azabicyclo[4.1.0]heptan-2-one and (1R,6R)-3-(4-fluoro-1-methyl-1H-indazol-5-yl)-1-((S)-2-(4-fluoro-3,5-dimethylphenyl)-4-methyl-4,5,6,7-tetrahydro-2H-pyrazolo[4,3-c]pyridin-3-yl)-3-azabicyclo[4.1.0]heptan-2-one

A mixture of tert-butyl (4S)-3-(3-(4-fluoro-1-methyl-1H-indazol-5-yl)-2-oxo-3-azabicyclo[4.1.0]heptan-1-yl)-2-(4-fluoro-3,5-dimethylphenyl)-4-methyl-2,4,6,7-tetrahydro-5H-pyrazolo[4,3-c]pyridine-5-carboxylate (97 mg, 0.16 mmol) and 4M HCl/dioxane (0.98 mL, 3.9 mmol) was stirred at 25° C. for 1 h. Then, Et₂O (10 mL) was added to mixture. The resulting solid precipitate was filtered and washed with Et₂O (2×10 mL) to provide title compounds as a mixture. MS (ESI) m/z 516.6 (pk 1), 516.6 (pk 2) [M+H]+.

### Step 4: 3-((1S,2S)-1-(2-((S)-3-((1S,6S)-3-(4-fluoro-1-methyl-1H-indazol-5-yl)-2-oxo-3-azabicyclo[4.1.0]heptan-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one and 3-((1S,2S)-1-(2-((S)-3-((1R,6R)-3-(4-fluoro-1-methyl-1H-indazol-5-yl)-2-oxo-3-azabicyclo[4.1.0]heptan-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one

To a solution of 3-(4-fluoro-1-methyl-1H-indazol-5-yl)-1-((S)-2-(4-fluoro-3,5-dimethylphenyl)-4-methyl-4,5,6,7-tetrahydro-2H-pyrazolo[4,3-c]pyridin-3-yl)-3-azabicyclo[4.1.0]heptan-2-one hydrochloride salt (82 mg, 0.15 mmol), 1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carboxylic acid (63 mg, 0.16 mmol) and HATU (68 mg, 0.18 mmol) in DMF (2.0 mL) was added DIPEA (77 mg, 0.59 mmol) at 23° C. The reaction mixture was stirred at 23° C. for 4 h. Then, the reaction mixture was purified by reverse phase prep-HPLC (5% to 100% MeCN/H₂O+0.1% TFA gradient) to provide the title compounds: Isomer A (faster eluting): ¹H NMR (CH₃OH-d4, 400 MHz): δH 8.08-8.13 (1H, m), 7.39-7.58 (3H, m), 7.16-7.30 (3H, m), 6.92-7.12 (0.5H, m), 6.68-6.85 (0.5H, m), 5.75-5.80 (0.6H, m), 5.30-5.38 (0.2H, m), 4.71-4.82 (0.5H, m), 4.439-4.41 (0.5H, m), 4.06-4.08 (5H, m), 3.37-3.77 (4H, m), 2.76-3.17 (4H, m), 2.28-2.40 (7H, m), 1.33-1.88 (13H, m), 1.00-1.58 (5H, m). MS (ESI) m/z: 882.0 [M+H]+. Isomer B (slower eluting): ¹H NMR (CH₃OH-d4, 400 MHz): δH 8.12 (1H, s), 7.43-7.54 (4H, m), 7.26-7.35 (2H, m), 7.00 (1H, br.s), 6.70-6.86 (1H, m), 5.79 (1H, br.s), 4.42-4.38 (1H, m), 4.03-4.11 (5H, m), 3.52-3.72 (4H, m), 3.11-3.13 (1H, m), 2.87-2.90 (2H, m), 2.67 (1H, br.s), 2.29-2.33 (6H, m), 2.22-2.24 (1H, m), 1.55-2.00 (12H, m), 0.81-1.32 (5H, m). MS (ESI) m/z: 882.0 [M+H]+. The following examples in table 14 were prepared using the procedures outlined in the synthesis of examples 156A and 156B.

TABLE 14

| | | | MS |
|---|---|---|---|
| Ex. | Structure | Name | (M + 1) |
| 158 Iso- mer A | 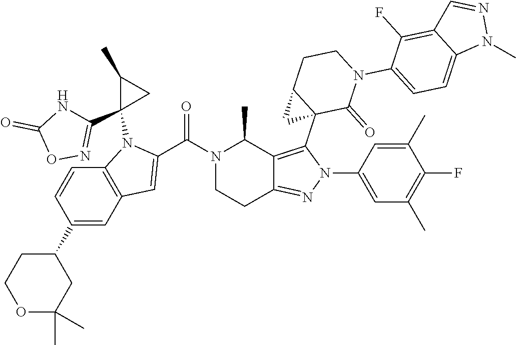 [SMILES: Cc1cc(-n2nc3c(c2[C@]2(C)C(=O)N(c4ccc5c(cnn5C)c4F)CC[C@H]2C)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | 3-((1S,2S)-1-(5-((S)-2,2- dimethyltetrahydro-2H-pyran-4-yl)- 2-((S)-3-((1R,6R)-3-(4-fluoro-1- methyl-1H-indazol-5-yl)-2-oxo-3- azabicyclo[4.1.0]heptan-1-yl)-2-(4- fluoro-3,5-dimethylphenyl)-4- methyl-4,5,6,7-tetrahydro-2H- pyrazolo[4,3-c]pyridine-5-carbonyl)- 1H-indol-1-yl)-2- methylcyclopropyl)-1,2,4-oxadiazol- 5(4H)-one or 3-((1S,2S)-1-(5-((S)- 2,2-dimethyltetrahydro-2H-pyran-4- yl)-2-((S)-3-((1S,6S)-3-(4-fluoro-1- methyl-1H-indazol-5-yl)-2-oxo-3- azabicyclo[4.1.0]heptan-1-yl)-2-(4- fluoro-3,5-dimethylphenyl)-4- methyl-4,5,6,7-tetrahydro-2H- pyrazolo[4,3-c]pyridine-5-carbonyl)- 1H-indol-1-yl)-2- methylcyclopropyl)-1,2,4-oxadiazol- 5(4H)-one | 910.6 |
| | or | | |
| | 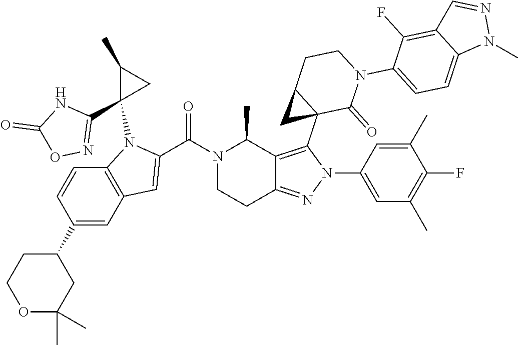 [SMILES: Cc1cc(-n2nc3c(c2[C@]24C[C@H]2CCN(c2ccc5c(cnn5C)c2F)C4=O)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | | |
| 158 Iso- mer B | 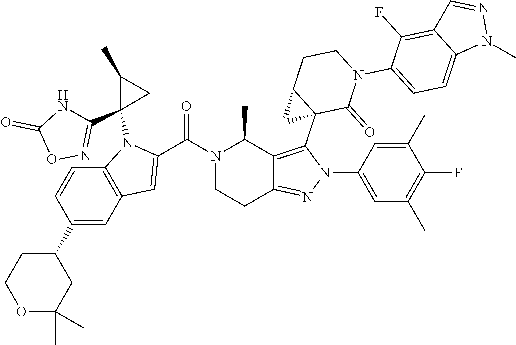 [SMILES: Cc1cc(-n2nc3c(c2[C@]2(C)C(=O)N(c4ccc5c(cnn5C)c4F)CC[C@H]2C)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | 3-((1S,2S)-1-(5-((S)-2,2- dimethyltetrahydro-2H-pyran-4-yl)- 2-((S)-3-((1R,6R)-3-(4-fluoro-1- methyl-1H-indazol-5-yl)-2-oxo-3- azabicyclo[4.1.0]heptan-1-yl)-2-(4- fluoro-3,5-dimethylphenyl)-4- methyl-4,5,6,7-tetrahydro-2H- pyrazolo[4,3-c]pyridine-5-carbonyl)- 1H-indol-1-yl)-2- methylcyclopropyl)-1,2,4-oxadiazol- 5(4H)-one or 3-((1S,2S)-1-(5-((S)- 2,2-dimethyltetrahydro-2H-pyran-4- yl)-2-((S)-3-((1S,6S)-3-(4-fluoro-1- methyl-1H-indazol-5-yl)-2-oxo-3- azabicyclo[4.1.0]heptan-1-yl)-2-(4- fluoro-3,5-dimethylphenyl)-4- methyl-4,5,6,7-tetrahydro-2H- pyrazolo[4,3-c]pyridine-5-carbonyl)- 1H-indol-1-yl)-2- methylcyclopropyl)-1,2,4-oxadiazol- 5(4H)-one | 910.6 |
| | or | | |
| | 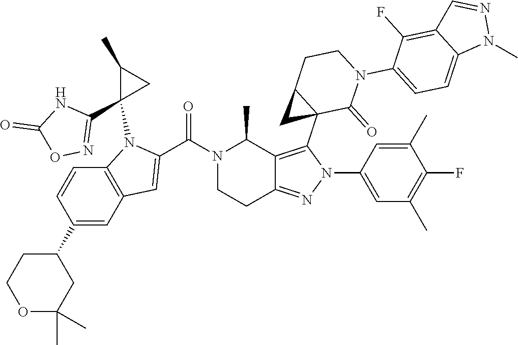 [SMILES: Cc1cc(-n2nc3c(c2[C@]24C[C@H]2CCN(c2ccc5c(cnn5C)c2F)C4=O)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | | |
| 159 Iso- mer A | 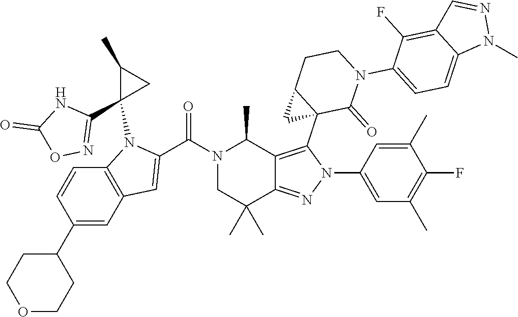 [SMILES: Cc1cc(-n2nc3c(c2[C@@]24C[C@@H]2CCN(c2ccc5c(cnn5C)c2F)C4=O)[C@H](C)N(C(=O)c2cc4cc(C5CCOCC5)ccc4n2[C@@]2(c4noc(=O)[nH]4)C[C@@H]2C)CC3(C)C)cc(C)c1F] | 3-((1S,2S)-1-(2-((S)-3-((1R,6R)-3- (4-fluoro-1-methyl-1H-indazol-5- yl)-2-oxo-3-azabicyclo[4.1.0]heptan- 1-yl)-2-(4-fluoro-3,5- dimethylphenyl)-4,7,7-trimethyl- 4,5,6,7-tetrahydro-2H-pyrazolo[4,3- c]pyridine-5-carbonyl)-5- (tetrahydro-2H-pyran-4-yl)-1H- indol-1-yl)-2-methylcyclopropyl)- 1,2,4-oxadiazol-5(4H)-one or 3- ((1S,2S)-1-(2-((S)-3-((1S,6S)-3-(4- fluoro-1-methyl-1H-indazol-5-y1)-2- oxo-3-azabicyclo[4.1.0]heptan-1-yl)- 2-(4-fluoro-3,5-dimethylphenyl)- 4,7,7-trimethyl-4,5,6,7-tetrahydro- 2H-pyrazolo[4,3-c]pyridine-5- carbonyl)-5-(tetrahydro-2H-pyran-4- yl)-1H-indol-1-yl)-2- methylcyclopropyl)-1,2,4-oxadiazol- 5(4H)-one | 910.6 |
| | or | | |
| | 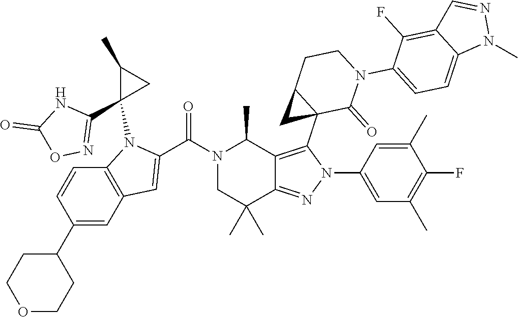 [SMILES: Cc1cc(-n2nc3c(c2[C@]24C[C@H]2CCN(c2ccc5c(cnn5C)c2F)C4=O)[C@H](C)N(C(=O)c2cc4cc(C5CCOCC5)ccc4n2[C@@]2(c4noc(=O)[nH]4)C[C@@H]2C)CC3(C)C)cc(C)c1F] | | |
| 159 Iso- mer B | 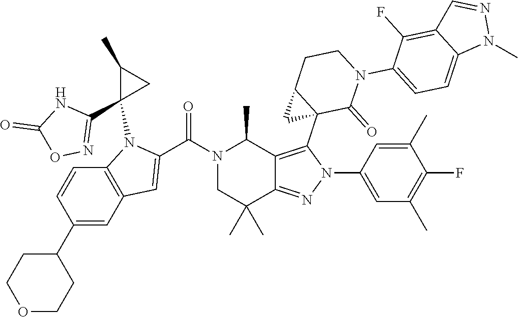 [SMILES: Cc1cc(-n2nc3c(c2[C@@]24C[C@@H]2CCN(c2ccc5c(cnn5C)c2F)C4=O)[C@H](C)N(C(=O)c2cc4cc(C5CCOCC5)ccc4n2[C@@]2(c4noc(=O)[nH]4)C[C@@H]2C)CC3(C)C)cc(C)c1F] | 3-((1S,2S)-1-(2-((S)-3-((1R,6R)-3- (4-fluoro-1-methyl-1H-indazol-5- yl)-2-oxo-3-azabicyclo[4.1.0]heptan- 1-yl)-2-(4-fluoro-3,5- dimethylphenyl)-4,7,7-trimethyl- 4,5,6,7-tetrahydro-2H-pyrazolo[4,3- c]pyridine-5-carbonyl)-5- (tetrahydro-2H-pyran-4-yl)-1H- indol-1-yl)-2-methylcyclopropyl)- 1,2,4-oxadiazol-5(4H)-one or 3- ((1S,2S)-1-(2-((S)-3-((1S,6S)-3-(4- fluoro-1-methyl-1H-indazol-5-yl)-2- oxo-3-azabicyclo[4.1.0]heptan-1-yl)- 2-(4-fluoro-3,5-dimethylphenyl)- 4,7,7-trimethyl-4,5,6,7-tetrahydro- 2H-pyrazolo[4,3-c]pyridine-5- carbonyl)-5-(tetrahydro-2H-pyran-4- yl)-1H-indol-1-yl)-2- methylcyclopropyl)-1,2,4-oxadiazol- 5(4H)-one | 910.6 |
| | or | | |
| | 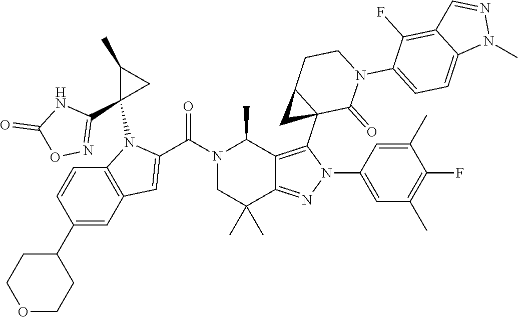 [SMILES: Cc1cc(-n2nc3c(c2[C@]24C[C@H]2CCN(c2ccc5c(cnn5C)c2F)C4=O)[C@H](C)N(C(=O)c2cc4cc(C5CCOCC5)ccc4n2[C@@]2(c4noc(=O)[nH]4)C[C@@H]2C)CC3(C)C)cc(C)c1F] | | |
| 225 Iso- mer A | 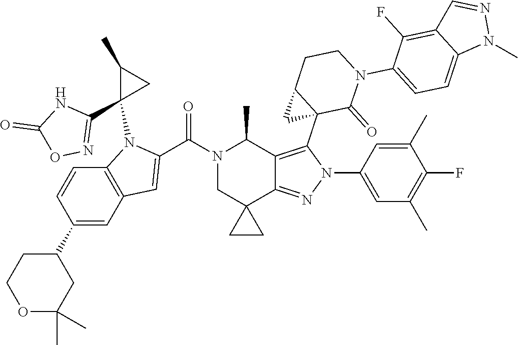 or 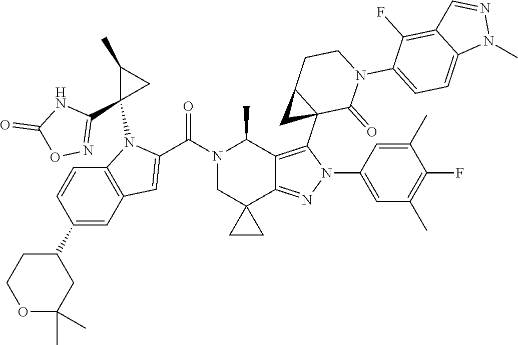 | 3-((1S,2S)-1-(5-((S)-2,2- dimethyltetrahydro-2H-pyran-4-yl)- 2-((S)-3′-((1R,6R)-3-(4-fluoro-1- methyl-1H-indazol-5-yl)-2-oxo-3- azabicyclo[4.1.0]heptan-1-yl)-2′-(4- fluoro-3,5-dimethylphenyl)-4′- methyl-2′,4′,5′,6′- tetrahydrospiro[cyclopropane-1,7′- pyrazolo[4,3-c]pyridine]-5′- carbonyl)-1H-indol-1-yl)-2- methylcyclopropyl)-1,2,4-oxadiazol- 5(4H)-one or 3-((1S,2S)-1-(5-((S)-2,2- dimethyltetrahydro-2H-pyran-4-yl)- 2-((S)-3′-((1S,6S)-3-(4-fluoro-1- methyl-1H-indazol-5-yl)-2-oxo-3- azabicyclo[4.1.0]heptan-1-yl)-2′-(4- fluoro-3,5-dimethylphenyl)-4′- methyl-2′,4′,5′,6′- tetrahydrospiro[cyclopropane-1,7′- pyrazolo[4,3-c]pyridine]-5′- carbonyl)-1H-indol-1-yl)-2- methylcyclopropyl)-1,2,4-oxadiazol- 5(4H)-one | 936.7 |
| 225 Iso- mer B | 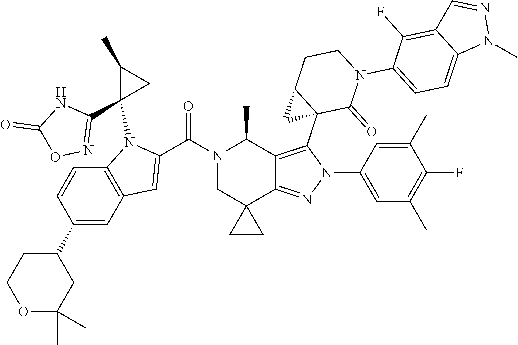 or 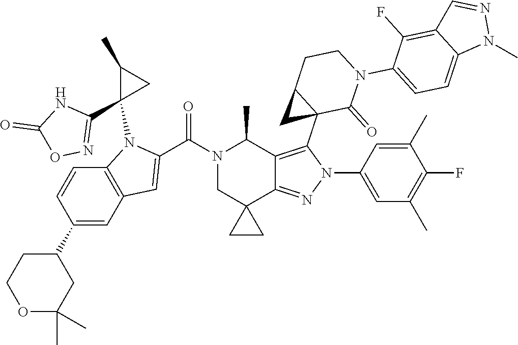 | 3-((1S,2S)-1-(5-((S)-2,2- dimethyltetrahydro-2H-pyran-4-yl)- 2-((S)-3′-((1R,6R)-3-(4-fluoro-1- methyl-1H-indazol-5-yl)-2-oxo-3- azabicyclo[4.1.0]heptan-1-yl)-2′-(4- fluoro-3,5-dimethylphenyl)-4′- methyl-2′,4′,5′,6′- tetrahydrospiro[cyclopropane-1,7′- pyrazolo[4,3-c]pyridine]-5′- carbonyl)-1H-indol-1-yl)-2- methylcyclopropyl)-1,2,4-oxadiazol- 5(4H)-one or 3-((1S,2S)-1-(5-((S)-2,2- dimethyltetrahydro-2H-pyran-4-yl)- 2-((S)-3′-((1S,6S)-3-(4-fluoro-1- methyl-1H-indazol-5-yl)-2-oxo-3- azabicyclo[4.1.0]heptan-1-yl)-2′-(4- fluoro-3,5-dimethylphenyl)-4′- methyl-2′,4′,5′,6′- tetrahydrospiro[cyclopropane-1,7′- pyrazolo[4,3-c]pyridine]-5′- carbonyl)-1H-indol-1-yl)-2- methylcyclopropyl)-1,2,4-oxadiazol- 5(4H)-one | 936.7 |
| 226 Iso- mer A | 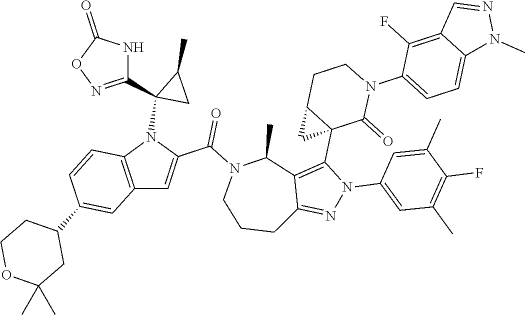 or 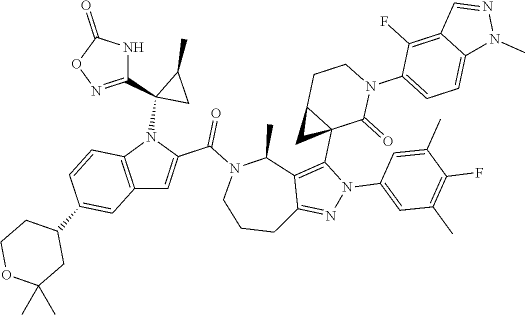 | 3-((1S,2S)-1-(5-((S)-2,2- dimethyltetrahydro-2H-pyran-4-yl)- 2-((S)-3-((1R,6R)-3-(4-fluoro-1- methyl-1H-indazol-5-yl)-2-oxo-3- azabicyclo[4.1.0]heptan-1-yl)-2-(4- fluoro-3,5-dimethylphenyl)-4- methyl-2,4,5,6,7,8- hexahydropyrazolo[4,3-c]azepine-5- carbonyl)-1H-indol-1-yl)-2- methylcyclopropyl)-1,2,4-oxadiazol- 5(4H)-one or 3-((1S,2S)-1-(5-((S)-2,2- dimethyltetrahydro-2H-pyran-4-yl)- 2-((S)-3-((1S,6S)-3-(4-fluoro-1- methyl-1H-indazol-5-yl)-2-oxo-3- azabicyclo[4.1.0]heptan-1-yl)-2-(4- fluoro-3,5-dimethylphenyl)-4- methyl-2,4,5,6,7,8- hexahydropyrazolo[4,3-c]azepine-5- carbonyl)-1H-indol-1-yl)-2- methylcyclopropyl)-1,2,4-oxadiazol- 5(4H)-one | 924.5 |
| 226 Iso- mer B | 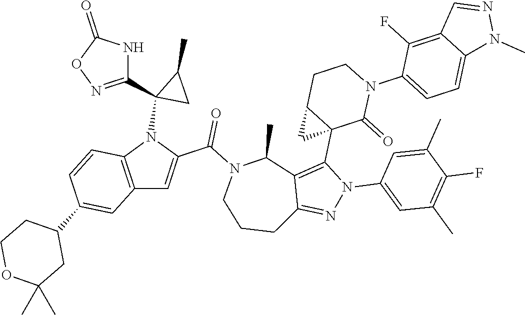 or 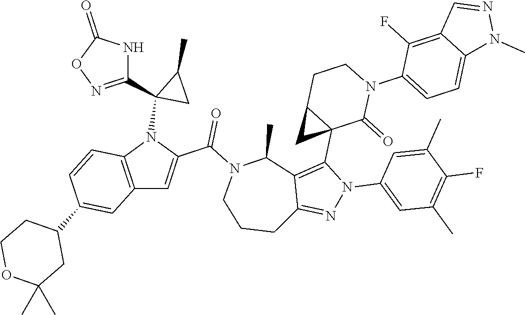 | 3-((1S,2S)-1-(5-((S)-2,2- dimethyltetrahydro-2H-pyran-4-yl)- 2-((S)-3-((1R,6R)-3-(4-fluoro-1- methyl-1H-indazol-5-yl)-2-oxo-3- azabicyclo[4.1.0]heptan-1-yl)-2-(4- fluoro-3,5-dimethylphenyl)-4- methyl-2,4,5,6,7,8- hexahydropyrazolo[4,3-c]azepine-5- carbonyl)-1H-indol-1-yl)-2- methylcyclopropyl)-1,2,4-oxadiazol- 5(4H)-one or 3-((1S,2S)-1-(5-((S)-2,2- dimethyltetrahydro-2H-pyran-4-yl)- 2-((S)-3-((1S,6S)-3-(4-fluoro-1- methyl-1H-indazol-5-yl)-2-oxo-3- azabicyclo[4.1.0]heptan-1-yl)-2-(4- fluoro-3,5-dimethylphenyl)-4- methyl-2,4,5,6,7,8- hexahydropyrazolo[4,3-c]azepine-5- carbonyl)-1H-indol-1-yl)-2- methylcyclopropyl)-1,2,4-oxadiazol- 5(4H)-one | 924.5 |

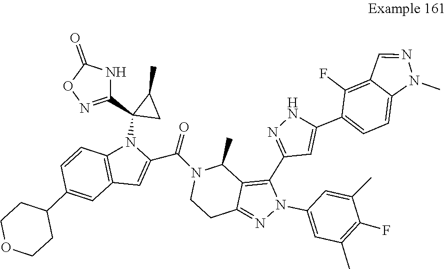 [SMILES: Cc1cc(-n2nc3c(c2-c2cc(-c4ccc5c(cnn5C)c4F)[nH]n2)[C@H](C)N(C(=O)c2cc4cc(C5CCOCC5)ccc4n2[C@@]2(c4noc(=O)[nH]4)C[C@@H]2C)CC3)cc(C)c1F]

### 3-((1S,2S)-1-(2-((S)-3-(5-(4-fluoro-1-methyl-1H-indazol-5-yl)-1H-pyrazol-3-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one

### Step 1: tert-butyl (S)-2-(4-fluoro-3,5-dimethylphenyl)-4-methyl-3-(4,4,5,5-tetramethyl-1,3,2-dioxaborolan-2-yl)-2,4,6,7-tetrahydro-5H-pyrazolo[4,3-c]pyridine-5-carboxylate

To a mixture of tert-butyl (S)-3-bromo-2-(4-fluoro-3,5-dimethylphenyl)-4-methyl-2,4,6,7-tetrahydro-5H-pyrazolo[4,3-c]pyridine-5-carboxylate (500 mg, 1.1 mmol) in THF (5.0 mL) was added n-BuLi (2.5M in hexane) (0.55 mL, 1.4 mmol) at −78° C. under an atmosphere of N₂. The reaction mixture was stirred at −78° C. for 0.5 h, then a solution of 2-isopropoxy-4,4,5,5-tetramethyl-1,3,2-dioxaborolane (42 mg, 2.3 mmol) in THF (2.0 mL) was added to the mixture. The mixture was stirred at −78° C. for 1 h, then quenched with H₂O (20 mL) at 0° C. and extracted with EtOAc (3×20 mL). The combined organic extracts were washed with brine (20 mL), dried over Na₂SO₄, filtered, and concentrated under reduced pressure to provide the title compound. MS (ESI) m/z: 485.9 [M+H+].

### Step 2: tert-butyl (S)-3-(5-bromo-1H-pyrazol-3-yl)-2-(4-fluoro-3,5-dimethylphenyl)-4-methyl-2,4,6,7-tetrahydro-5H-pyrazolo[4,3-c]pyridine-5-carboxylate

To a solution of (S)-(5-(tert-butoxycarbonyl)-2-(4-fluoro-3,5-dimethylphenyl)-4-methyl-4,5,6,7-tetrahydro-2H-pyrazolo[4,3-c]pyridin-3-yl)boronic acid (540 mg, 1.34 mmol), 3,5-dibromo-1H-pyrazole (280 mg, 1.24 mmol), and potassium phosphate tribasic (1.05 g, 4.96 mmol) in t-AmOH (4.0 mL) and water (1.0 mL) was added [1,1′-Bis(di-tert-butylphosphino)ferrocene]dichloropalladium(II) (81 mg, 0.12 mmol) under an atmosphere of N₂. The resulting mixture was stirred at 60° C. for 16 h. Then, the reaction mixture was purified by prep-HPLC (60 to 80% MeCN/H₂O+0.1% TFA). MS (ESI) m/z: 506.1 [M+H+].

### Step 3: tert-butyl (S)-3-(5-(4-fluoro-1-methyl-1H-indazol-5-yl)-1H-pyrazol-3-yl)-2-(4-fluoro-3,5-dimethylphenyl)-4-methyl-2,4,6,7-tetrahydro-5H-pyrazolo[4,3-c]pyridine-5-carboxylate

To a solution of tert-butyl (S)-3-(5-bromo-1H-pyrazol-3-yl)-2-(4-fluoro-3,5-dimethylphenyl)-4-methyl-2,4,6,7-tetrahydro-5H-pyrazolo[4,3-c]pyridine-5-carboxylate (14 mg, 0.028 mmol), 4-fluoro-1-methyl-5-(4,4,5,5-tetramethyl-1,3,2-dioxaborolan-2-yl)-1H-indazole (12 mg, 0.042 mmol), and potassium phosphate tribasic (18 mg, 0.083 mmol) in t-AmOH/H2O (5:1) (0.50 mL) was added [1,1′-Bis(di-tert-butylphosphino)ferrocene]dichloropalladium(II) (1.8 mg, 2.8 μmol) under an atmosphere of N₂. The resulting mixture was stirred at 80° C. for 16 h, then quenched with H₂O (10 mL) and extracted with EtOAc (3×10 mL). The combined organic extracts were dried over Na₂SO₄ and concentrated under reduced pressure. The crude residue was purified by prep-TLC (SiO2; 33% EtOAc/Pet.ether) to provide the title compound. MS (ESI) m/z: 574.3 [M+H+].

### Steps 4 and 5: 3-((1S,2S)-1-(2-((S)-3-(5-(4-fluoro-1-methyl-1H-indazol-5-yl)-1H-pyrazol-3-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one

These steps were performed in a similar fashion as described for example 1 to provide the title compound as a TFA salt. ¹H NMR (400 MHz, methanol-d4) δ 8.15-8.05 (m, 1H), 7.71 (dd, J=6.9, 8.6 Hz, 0.5H), 7.61-7.41 (m, 4H), 7.38 (d, J=8.8 Hz, 0.5H), 7.27 (s, 1H), 7.18-7.06 (m, 2H), 6.88 (d, J=8.1 Hz, 1H), 6.39-6.27 (m, 0.5H), 6.09-6.03 (m, 0.5H), 6.03-5.98 (m, 0.5H), 5.78-5.71 (m, 0.5H), 5.76-5.67 (m, 1H), 4.51-4.40 (m, 0.5H), 4.11-4.03 (m, 5H), 3.79-3.65 (m, 1H), 3.59 (dt, J=2.9, 11.3 Hz, 2H), 3.27-3.17 (m, 0.5H), 3.02-2.85 (m, 2H), 2.32-2.16 (m, 7H), 1.92-1.74 (m, 6H), 1.67-1.54 (m, 3H), 1.52-1.45 (m, 1H), 1.34-1.26 (m, 1H), 1.22 (br d, J=5.6 Hz, 1H), 1.10 (br d, J=5.4 Hz, 1H). MS (ESI) m/z: 839.3 [M+H+].

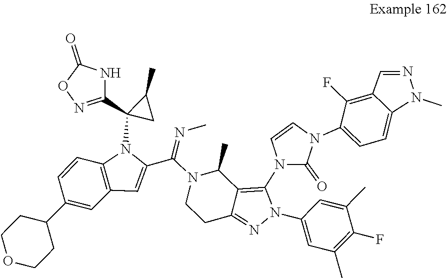 [SMILES: C/N=C(/c1cc2cc(C3CCOCC3)ccc2n1[C@@]1(c2noc(=O)[nH]2)C[C@@H]1C)N1CCc2nn(-c3cc(C)c(F)c(C)c3)c(-n3ccn(-c4ccc5c(cnn5C)c4F)c3=O)c2[C@@H]1C]

### 3-((1S,2S)-1-(2-((Z)—((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-2,4,6,7-tetrahydro-5H-pyrazolo[4,3-c]pyridin-5-yl)(methylimino)methyl)-5-(tetrahydro-2H-pyran-4-yl)-1H-indol-1-yl)-2-methylcyclopropyl)-1,2,4-oxadiazol-5(4H)-one

### Step 1: 3-((1S,2S)-1-(2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonothioyl)-5-(tetrahydro-2H-pyran-4-yl)-1H-indol-1-yl)-2-methylcyclopropyl)-1,2,4-oxadiazol-5(4H)-one

A solution of 3-((1S,2S)-1-(2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one (226 mg, 0.264 mmol) and Lawesson's reagent (321 mg, 0.793 mmol) in toluene (5 mL) was stirred at 100° C. for 3 h, then concentrated under reduced pressure. The crude residue was purified by prep-HPLC (65% to 85% MeCN/H₂O+0.1% TFA gradient) to afford the title compound. MS (ESI) m/z: 871.1 [M+H+].

### Step 2: 3-((1S,2S)-1-(2-((Z)—((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-2,4,6,7-tetrahydro-5H-pyrazolo[4,3-c]pyridin-5-yl)(methylimino)methyl)-5-(tetrahydro-2H-pyran-4-yl)-1H-indol-1-yl)-2-methylcyclopropyl)-1,2,4-oxadiazol-5(4H)-one

To a solution of 3-((1S,2S)-1-(2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonothioyl)-5-(tetrahydro-2H-pyran-4-yl)-1H-indol-1-yl)-2-methylcyclopropyl)-1,2,4-oxadiazol-5(4H)-one (10 mg, 0.011 mmol) in THF (1 mL) was added methanamine (0.36 mg, 0.011 mmol) and TEA (1.6 μL, 0.011 mmol). The mixture was stirred at 70° C., then cooled to rt, filtered, and concentrated under reduced pressure. The crude residue was purified by prep-HPLC (3600 to 56% MeCN/H₂O+0.1% TFA gradient) to provide the title compound as a TFA salt. ¹H NMR (400 MHz, MeOD) δ 8.18 (brd, J=3.9 Hz, 1H), 7.51-7.42 (m, 3H), 7.35 (br d, J=8.2 Hz, 1H), 7.26-7.11 (m, 3H), 6.97-6.51 (m, 3H), 4.16-4.00 (m, 6H), 3.68-3.53 (m, 3H), 2.93-2.79 (m, 4H), 2.31-2.26 (m, 6H), 1.87-1.78 (m, 4H), 1.51-1.46 (m, 1H), 1.38-1.26 (in, 8H). MS (ESI): m/z 868.3 [M+H+]. The following examples in table 15 were prepared according to the procedure outlined in the synthesis of example 162.

TABLE 15

| | | | MS |
|---|---|---|---|
| | | | (M + |
| Ex. | Structure | Name | 1) |
| 163 | 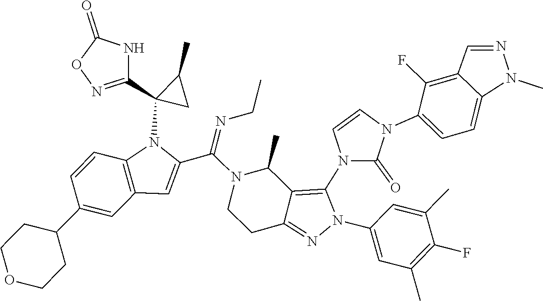 [SMILES: CC/N=C(/c1cc2cc(C3CCOCC3)ccc2n1[C@@]1(c2noc(=O)[nH]2)C[C@@H]1C)N1CCc2nn(-c3cc(C)c(F)c(C)c3)c(-n3ccn(-c4ccc5c(cnn5C)c4F)c3=O)c2[C@@H]1C] | 3-((1S,2S)-1-(2-((Z)- (ethylimino)((S)-3-(3-(4-fluoro- 1-methyl-1H-indazol-5-yl)-2- oxo-2,3-dihydro-1H-imidazol- 1-yl)-2-(4-fluoro-3,5- dimethylphenyl)-4-methyl- 2,4,6,7-tetrahydro-5H- pyrazolo[4,3-c]pyridin-5- yl)methyl)-5-(tetrahydro-2H- pyran-4-yl)-1H-indol-1-yl)-2- methylcyclopropyl)-1,2,4- oxadiazol-5(4H)-one | 882.3 |
| 164 | 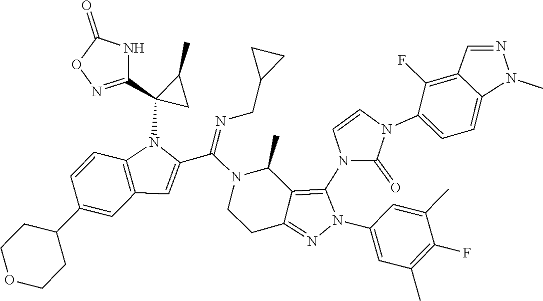 [SMILES: Cc1cc(-n2nc3c(c2-n2ccn(-c4ccc5c(cnn5C)c4F)c2=O)[C@H](C)N(/C(=N\CC2CC2)c2cc4cc(C5CCOCC5)ccc4n2[C@@]2(c4noc(=O)[nH]4)C[C@@H]2C)CC3)cc(C)c1F] | 3-((1S,2S)-1-(2-((Z)- ((cyclopropylmethyl)imino)((S)- 3-(3-(4-fluoro-1-methyl-1H- indazol-5-yl)-2-oxo-2,3- dihydro-1H-imidazol-1-yl)-2- (4-fluoro-3,5-dimethylphenyl)- 4-methyl-2,4,6,7-tetrahydro- 5H-pyrazolo[4,3-c]pyridin-5- yl)methyl)-5-(tetrahydro-2H- pyran-4-yl)-1H-indol-1-yl)-2- methylcyclopropyl)-1,2,4- oxadiazol-5(4H)-one | 908.3 |

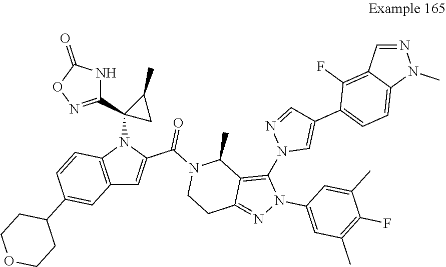 [SMILES: Cc1cc(-n2nc3c(c2-n2cc(-c4ccc5c(cnn5C)c4F)cn2)[C@H](C)N(C(=O)c2cc4cc(C5CCOCC5)ccc4n2[C@@]2(c4noc(=O)[nH]4)C[C@@H]2C)CC3)cc(C)c1F]

### 3-((1S,2S)-1-(2-((S)-3-(4-(4-fluoro-1-methyl-1H-indazol-5-yl)-1H-pyrazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one

### Step 1: (S)-2-(4-fluoro-3,5-dimethylphenyl)-4-methyl-3-(1H-pyrazol-1-yl)-4,5,6,7-tetrahydro-2H-pyrazolo[4,3-c]pyridine

To a solution of di-tert-butyl (S)-1-(5-(tert-butoxycarbonyl)-2-(4-fluoro-3,5-dimethylphenyl)-4-methyl-4,5,6,7-tetrahydro-2H-pyrazolo[4,3-c]pyridin-3-yl)hydrazine-1,2-dicarboxylate (75 mg, 0.13 mmol) in 2M HCl MeOH/MeOH(1:1) (2.0 mL) was added 1,1,3,3-tetramethoxypropane (42 mg, 0.25 mmol), and the reaction mixture was stirred for 1 h at 90° C. The reaction was purified by reverse-phase HPLC (20% to 40% MeCN/water+0.1% TFA) to provide the title compound. LCMS (ESI) m/z: 326.1 [M+H]+.

### Step 2: (S)-3-(4-bromo-1H-pyrazol-1-yl)-2-(4-fluoro-3,5-dimethylphenyl)-4-methyl-4,5,6,7-tetrahydro-2H-pyrazolo[4,3-c]pyridine

To a solution of (S)-2-(4-fluoro-3,5-dimethylphenyl)-4-methyl-3-(1H-pyrazol-1-yl)-4,5,6,7-tetrahydro-2H-pyrazolo[4,3-c]pyridine (39 mg, 0.12 mmol) in AcOH (1.0 mL) was added monopyridin-1-ium tribromide (230 mg, 0.72 mmol) and stirred for 16 h at 60° C. The reaction mixture was cooled to rt, concentrated under reduced pressure, then purified by reverse-phase HPLC (30% to 50% MeCN/water+0.1% TFA) to afford the title compound. LCMS (ESI) m/z: 404.0, 406.0 [M+H]+.

### Step 3: (2S,4S)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-6,11-dioxo-N-(pyrrolidin-3-yl)-1,2,3,4,6,11-hexahydrotetracene-2-carboxamide

To a solution of (S)-3-(4-bromo-1H-pyrazol-1-yl)-2-(4-fluoro-3,5-dimethylphenyl)-4-methyl-4,5,6,7-tetrahydro-2H-pyrazolo[4,3-c]pyridine (30 mg, 0.074 mmol) and 4-fluoro-1-methyl-5-(4,4,5,5-tetramethyl-1,3,2-dioxaborolan-2-yl)-1H-indazole (27 mg, 0.096 mmol) in 2-methyl-2-butanol (0.50 mL) was added PdCl₂(dtbpf) (4.8 mg, 7.4 μmol) under an atmosphere of N₂. Then, potassium phosphate tribasic (79 mg, 0.37 mmol) in water (0.13 mL) was added to the mixture. The resulting mixture was stirred at 80° C. for 16 h. The reaction mixture was diluted by water (10 mL) and extracted with EtOAc (3×10 mL). The combined organic layers were dried over Na₂SO₄, filtered, and concentrated under reduced pressure. The crude residue was purified by reverse-phase HPLC (30% to 50% MeCN/water+0.1% TFA) to afford the title compound. MS (ESI) m/z: 474.1 [M+H]+.

### Step 4: 3-((1S,2S)-1-(2-((S)-3-(4-(4-fluoro-1-methyl-1H-indazol-5-yl)-1H-pyrazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one

This step was performed for 2S,4S)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-6,11-dioxo-N-(pyrrolidin-3-yl)-1,2,3,4,6,11-hexahydrotetracene-2-carboxamide in a similar fashion as described for example 1 to provide the title compound. ¹H NMR (400 MHz, methanol-d4) δ=8.33-8.22 (m, 1H), 8.12 (s, 1H), 8.05-7.66 (m, 1H), 7.60-7.25 (m, 5H), 7.05-6.81 (m, 3H), 5.87-5.77 (m, 1H), 5.51-5.39 (m, 1H), 4.57-4.46 (m, 1H), 4.12-3.99 (m, 6H), 3.61-3.54 (m, 2H), 3.05-2.99 (m, 1H), 2.92-2.84 (m, 1H), 2.22-2.16 (m, 6H), 1.88-1.76 (m, 6H), 1.56 (td, J=1.6, 3.2 Hz, 2H), 1.44-1.38 (m, 2H), 1.33-1.29 (m, 1H), 1.29-1.10 (m, 4H). LCMS (ESI) m/z: 839.3 [M+H]+.

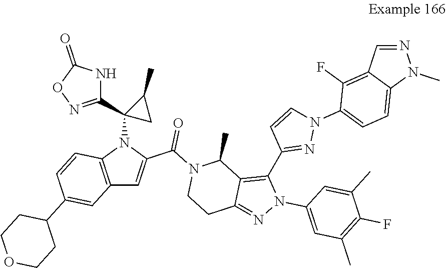 [SMILES: Cc1cc(-n2nc3c(c2-c2ccn(-c4ccc5c(cnn5C)c4F)n2)[C@H](C)N(C(=O)c2cc4cc(C5CCOCC5)ccc4n2[C@@]2(c4noc(=O)[nH]4)C[C@@H]2C)CC3)cc(C)c1F]

### (2S,4S)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-6,11-dioxo-N-(pyrrolidin-3-yl)-1,2,3,4,6,11-hexahydrotetracene-2-carboxamide

### Step 1: tert-butyl (S)-2-(4-fluoro-3,5-dimethylphenyl)-4-methyl-3-(1H-pyrazol-3-yl)-2,4,6,7-tetrahydro-5H-pyrazolo[4,3-c]pyridine-5-carboxylate

Tert-butyl (S)-3-bromo-2-(4-fluoro-3,5-dimethylphenyl)-4-methyl-2,4,6,7-tetrahydro-5H-pyrazolo[4,3-c]pyridine-5-carboxylate (300 mg, 0.684 mmol), (1H-pyrazol-3-yl)boronic acid (115 mg, 1.03 mmol), potassium phosphate tribasic (581 mg, 2.74 mmol), PdCl₂(dtbpf) (89.0 mg, 0.137 mmol) in t-AmOH (5.0 mL) and water (1.0 mL) at 15° C. was stirred at 80° C. for 16 h under an atmosphere of nitrogen. To the reaction mixture was added additional potassium phosphate (581 mg, 2.74 mmol), (1H-pyrazol-3-yl) boronic acid (153 mg, 1.369 mmol), and PdCl₂(dtbpf) (89 mg, 0.137 mmol), and the resulting mixture was stirred at 80° C. for 16 h. Then, the mixture was cooled to rt, filtered, diluted with water (20 mL) and then extracted with EtOAc (3×20 mL). The combined organic layers were washed with brine (20 mL), dried over Na₂SO₄, filtered, and concentrated under reduced pressure. The crude residue was purified by reverse-phase HPLC (62% to 92% MeCN/water+0.1% TFA) to afford the title compound. LCMS (ESI) m/z: 426.1 [M+H]+.

### Step 2: tert-butyl (S)-3-(1-(4-fluoro-1-methyl-1H-indazol-5-yl)-1H-pyrazol-3-yl)-2-(4-fluoro-3,5-dimethylphenyl)-4-methyl-2,4,6,7-tetrahydro-5H-pyrazolo[4,3-c]pyridine-5-carboxylate

To a solution of tert-butyl (S)-2-(4-fluoro-3,5-dimethylphenyl)-4-methyl-3-(1H-pyrazol-3-yl)-2,4,6,7-tetrahydro-5H-pyrazolo[4,3-c]pyridine-5-carboxylate (20 mg, 0.047 mmol), 5-bromo-4-fluoro-1-methyl-1H-indazole (22 mg, 0.094 mmol), (1S,2S)—N1,N2-dimethylcyclohexane-1,2-diamine (3.3 mg, 0.024 mmol) and K₂CO₃ (19 mg, 0.14 mmol) in NMP (0.5 mL) was added copper(I) iodide (1.8 mg, 9.4 μmol). The reaction mixture was stirred at 130° C. under N₂ for 16 h. After cooling to rt, the reaction mixture was poured into water (10 mL) and extracted with EtOAc (3×10 mL×3). The combined organic layers were washed with brine (10 mL), dried over Na₂SO₄, concentrated under reduced pressure. The crude residue was purified by prep-TLC (SiO₂, 33% EtOAc/Pet. ether) to afford the title compound. LCMS (ESI) m/z: 574.2 [M+H]+.

### Step 3 and 4: (2S,4S)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-6,11-dioxo-N-(pyrrolidin-3-yl)-1,2,3,4,6,11-hexahydrotetracene-2-carboxamide

These steps were performed for tert-butyl (S)-3-(1-(4-fluoro-1-methyl-1H-indazol-5-yl)-1H-pyrazol-3-yl)-2-(4-fluoro-3,5-dimethylphenyl)-4-methyl-2,4,6,7-tetrahydro-5H-pyrazolo[4,3-c]pyridine-5-carboxylate in a similar fashion as described for example 1 to provide the title compound. ¹H NMR (400 MHz, methanol-d4) δ=8.17 (br d, J=14.8 Hz, 1H), 8.10-7.90 (m, 1H), 7.88-7.53 (m, 2H), 7.50 (s, 2H), 7.33-7.09 (m, 4H), 6.97-6.86 (m, 1H), 6.13-6.00 (m, 1H), 6.13-5.89 (m, 1H), 4.14 (s, 3H), 4.09-4.02 (m, 2H), 3.61-3.55 (m, 2H), 3.04-2.98 (m, 1H), 2.91-2.82 (m, 1H), 2.30 (br d, J=6.6 Hz, 7H), 1.85-1.79 (m, 3H), 1.76 (br d, J=6.4 Hz, 2H), 1.64 (br d, J=6.6 Hz, 3H), 1.60-1.54 (m, 1H), 1.29 (s, 3H), 1.14-1.03 (m, 1H), 1.10-0.89 (m, 1H), 1.13-0.88 (m, 1H). LCMS (ESI) m/z: 839.3 [M+H]+.

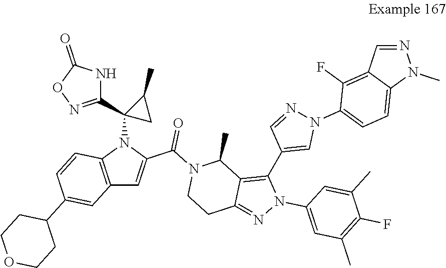 [SMILES: Cc1cc(-n2nc3c(c2-c2cnn(-c4ccc5c(cnn5C)c4F)c2)[C@H](C)N(C(=O)c2cc4cc(C5CCOCC5)ccc4n2[C@@]2(c4noc(=O)[nH]4)C[C@@H]2C)CC3)cc(C)c1F]

### 3-((1S,2S)-1-(2-((S)-3-(1-(4-fluoro-1-methyl-1H-indazol-5-yl)-1H-pyrazol-4-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one

These steps were performed for tert-butyl (S)-3-bromo-2-(4-fluoro-3,5-dimethylphenyl)-4-methyl-2,4,6,7-tetrahydro-5H-pyrazolo[4,3-c]pyridine-5-carboxylate and (1H-pyrazol-4-yl)boronic acid in a similar fashion as described for example 166 to provide the title compound. ¹H NMR (400 MHz, methanol-d4) δ 8.19 (s, 1H), 8.11-8.08 (m, 1H), 7.78-7.71 (m, 1H), 7.65 (s, 1H), 7.55-7.49 (m, 3H), 7.27 (d, J=9.2 Hz, 1H), 7.14-7.06 (m, 2H), 6.89 (s, 1H), 6.13-5.88 (m, 1H), 4.52-4.43 (m, 1H), 4.08-4.05 (m, 2H), 3.74-3.70 (m, 2H), 3.59-3.55 (m, 2H), 3.23-3.21 (m, 1H), 2.94-2.89 (m, 1H), 2.28 (s, 3H), 2.23 (s, 2H), 1.81 (d, J=10.8 Hz, 4H), 1.51 (d, J=6.4 Hz, 2H), 1.36 (d, J=3.2 Hz, 9H), 1.21-1.11 (m, 2H). LCMS (ESI) m/z: 839.3 [M+H]+.

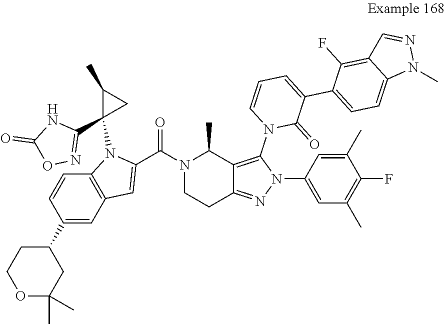 [SMILES: Cc1cc(-n2nc3c(c2-n2cccc(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F]

### 3-((1S,2S)-1-(5-((S)-2,2-dimethyltetrahydro-2H-pyran-4-yl)-2-((S)-3-(3-(4-fluoro-1-methyl-1H-indazol-5-yl)-2-oxopyridin-1(2H)-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one

### Step 1: tert-butyl (S)-2-(4-fluoro-3,5-dimethylphenyl)-3-(3-(methoxycarbonyl)-2-oxopyridin-1(2H)-yl)-4-methyl-2,4,6,7-tetrahydro-5H-pyrazolo[4,3-c]pyridine-5-carboxylate

To a solution of methyl 2-oxo-2H-pyran-3-carboxylate (100 mg, 0.65 mmol) in DMF (2.0 mL) under a N₂ atmosphere at 0° C. was added tert-butyl (S)-3-amino-2-(4-fluoro-3,5-dimethylphenyl)-4-methyl-2,4,6,7-tetrahydro-5H-pyrazolo[4,3-c]pyridine-5-carboxylate (243 mg, 0.649 mmol). After 2 h, the reaction mixture was allowed to warm to 25° C. and stirred for 15 min. Then, 1-(3-dimethylaminopropyl)-3-ethylcarbodiimide (130 mg, 0.84 mmol) and DMAP (20 mg, 0.16 mmol) were added. The reaction mixture was stirred at 25° C. for 12 h, then at 100° C. for 13 h by microwave heating. The reaction mixture was purified by reverse-phase HPLC (45 to 65% MeCN/H₂O+0.1% TFA) to afford the title compound. MS (ESI) m/z: 511.2 [M+H]+.

### Step 2: (S)-1-(5-(tert-butoxycarbonyl)-2-(4-fluoro-3,5-dimethylphenyl)-4-methyl-4,5,6,7-tetrahydro-2H-pyrazolo[4,3-c]pyridin-3-yl)-2-oxo-1,2-dihydropyridine-3-carboxylic acid

To a solution of tert-butyl (S)-2-(4-fluoro-3,5-dimethylphenyl)-3-(3-(methoxycarbonyl)-2-oxopyridin-1(2H)-yl)-4-methyl-2,4,6,7-tetrahydro-5H-pyrazolo[4,3-c]pyridine-5-carboxylate (10 mg, 0.020 mmol) in MeOH/THF(1:1) (0.5 mL) was added lithium hydroxide monohydrate (2.5 mg, 0.059 mmol) and the reaction mixture was stirred at 25° C. for 1 h. The reaction mixture was quenched by 1 M aq. HCl (1 mL), and extracted with EtOAc (3×1 mL). Then, the organic layers were combined, dried over Na₂SO₄, filtered and concentrated under reduced pressure to provide the title compound. MS (ESI) m/z: 497.2 [M+H]+.

### Step 3: tert-butyl (S)-3-(3-(4-fluoro-1-methyl-1H-indazol-5-yl)-2-oxopyridin-1(2H)-y)-2-(4-fluoro-3,5-dimethylphenyl)-4-methyl-2,4,6,7-tetrahydro-5H-pyrazolo[4,3-c]pyridine-5-carboxylate

To a solution of (S)-1-(5-(tert-butoxycarbonyl)-2-(4-fluoro-3,5-dimethylphenyl)-4-methyl-4,5,6,7-tetrahydro-2H-pyrazolo[4,3-c]pyridin-3-yl)-2-oxo-1,2-dihydropyridine-3-carboxylic acid (12 mg, 0.024 mmol), palladium(II) chloride (0.43 mg, 2.4 μmol), triphenylphosphine (1.3 mg, 4.8 μmol), and silver carbonate (13 mg, 0.048 mmol) in toluene/DMA (9:1, 0.20 mL) was added 5-bromo-4-fluoro-1-methyl-1H-indazole (11 mg, 0.048 mmol). The resulting mixture was stirred at 150° C. via microwave heating for 5 h. The reaction mixture was concentrated under reduced pressure, then the crude residue was purified by reverse-phase HPLC (58 to 78% MeCN/H2O 0.1% TFA) to afford the title compound. MS (ESI) m/z: 601.4 [M+H]+.

### Step 4: (S)-3-(4-fluoro-1-methyl-1H-indazol-5-yl)-1-(2-(4-fluoro-3,5-dimethylphenyl)-4-methyl-4,5,6,7-tetrahydro-2H-pyrazolo[4,3-c]pyridin-3-yl)pyridin-2(1H)-one

A solution of tert-butyl (S)-3-(3-(4-fluoro-1-methyl-1H-indazol-5-yl)-2-oxopyridin-1(2H)-yl)-2-(4-fluoro-3,5-dimethylphenyl)-4-methyl-2,4,6,7-tetrahydro-5H-pyrazolo[4,3-c]pyridine-5-carboxylate (16 mg, 0.027 mmol) in 2M HCl/dioxane (0.50 mL) was stirred at 25° C. for 16 h. Then, the reaction mixture was concentrated under reduced pressure to afford the title compound, which was used in the next step without further purification. MS (ESI) m/z: 501.3 [M+H]+.

### Step 5: 3-((1S,2S)-1-(5-((S)-2,2-dimethyltetrahydro-2H-pyran-4-yl)-2-((S)-3-(3-(4-fluoro-1-methyl-1H-indazol-5-yl)-2-oxopyridin-1(2H)-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one

To a solution of 5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carboxylic acid (15 mg, 0.036 mmol) and (S)-3-(4-fluoro-1-methyl-1H-indazol-5-yl)-1-(2-(4-fluoro-3,5-dimethylphenyl)-4-methyl-4,5,6,7-tetrahydro-2H-pyrazolo[4,3-c]pyridin-3-yl)pyridin-2(1H)-one (13 mg, 0.026 mmol) in DMF (0.50 mL) was added N-ethyl-N-isopropylpropan-2-amine (10 mg, 0.078 mmol). The mixture was stirred at 25° C. for 2 min. Then, HATU (15 mg, 0.039 mmol) was added, and the resulting mixture was stirred at 25° C. for 3 h. The reaction mixture was purified by reverse-phase HPLC (70 to 100% MeCN/H2O+0.1% TFA) to afford the title compound. ¹H NMR (400 MHz, acetonitrile-d3) δ 11.53-11.36 (m, 1H), 8.12-8.01 (m, 0.7H), 7.86 (s, 0.3H), 7.67-7.44 (m, 4H), 7.43-7.17 (m, 3H), 7.12-6.95 (m, 2H), 6.85-6.70 (m, 1H), 6.48 (t, J=6.9 Hz, 0.3H), 6.34 (t, J=6.9 Hz, 0.3H), 6.24 (t, J=6.9 Hz, 0.4H), 5.71-5.58 (m, 0.6H), 5.37-5.05 (m, 0.4H), 4.90-4.73 (m,.4H), 4.49-4.39 (m, 0.6H), 4.09-4.00 (m, 3H), 3.81-3.70 (m, 2H), 3.67-3.53 (m, 1H), 3.17-2.89 (m, 3H), 2.19-2.09 (m, 6H), 1.73-1.68 (m, 2H), 1.58-1.51 (m, 3H), 1.47-1.40 (m, 2H), 1.30-1.27 (m, 3H), 1.21-1.08 (m, 6H), 0.99-0.88 (m, 3H). MS (ESI) m/z: 894.3 [M+H]+.

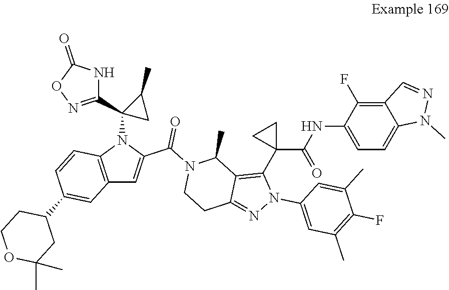

### 1-((S)-5-(5-((S)-2,2-dimethyltetrahydro-2H-pyran-4-yl)-1-((1 S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)-N-(4-fluoro-1-methyl-1H-indazol-5-yl)cyclopropane-1-carboxamide

### Step 1: methyl 2-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)acrylate

To a reaction vessel with methyl 2-((4S)-5-(5-(2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)acrylate (100 mg, 138 mol), potassium carbonate (19.1 mg, 138 mol), and paraformaldehyde (8.29 mg, 276 mol) was added DMF (1.4 mL). The reaction mixture was stirred at 100° C. for 1 h. Then the mixture was cooled to rt and purified by silica gel chromatography (0 to 100% EtOAc/Hexanes) to provide the title compound. MS (ESI) m/z 737.6 [M+H]+.

### Step 2: 2-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)acrylic acid

This step was performed in a similar fashion as described for example Xe from methyl 2-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)acrylate to provide the title compound. MS (ESI) m/z 723.6 [M+H]+.

### Step 3: 2-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)-N-(4-fluoro-1-methyl-1H-indazol-5-yl)acrylamide

This step was performed in a similar fashion as described for example Xe from 2-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)acrylic acid to provide the title compound. MS (ESI) m/z 870.6 [M+H]+.

### Step 4: 1-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)-N-(4-fluoro-1-methyl-1H-indazol-5-yl)cyclopropane-1-carboxamide

To a stirred suspension of trimethyl(oxo)sulfonium iodide (3.7 mg, 17 mol), 1-methyl-2,3,4,6,7,8-hexahydro-1H-pyrimido[1,2-a]pyrimidine (3.0 μL, 20 mol) in MeCN (100 L) was added 2-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)-N-(4-fluoro-1-methyl-1H-indazol-5-yl)acrylamide (13 mg, 15 mol) as a suspension in MeCN (900 L). The reaction mixture was stirred at 60° C. for 1 h. Then, the reaction mixture was concentrated under reduced pressure and purified by reverse-phase HPLC (55% to 75% MeCN/H2O+0.1% formic acid gradient) to provide the title compound. MS (ESI) m/z 884.4 [M+H]+.

The following examples in Table 16 were prepared using the procedures outlined in the synthesis of Example 169.

TABLE 16

| | | | MS |
|---|---|---|---|
| Ex. | Structure | Name | (M + 1) |
| 227 | 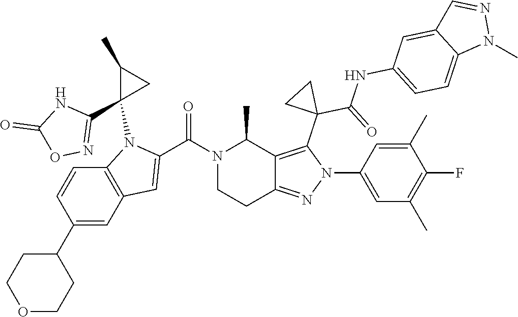 [SMILES: Cc1cc(-n2nc3c(c2C2(C(=O)Nc4ccc5c(cnn5C)c4)CC2)[C@H](C)N(C(=O)c2cc4cc(C5CCOCC5)ccc4n2[C@@]2(c4noc(=O)[nH]4)C[C@@H]2C)CC3)cc(C)c1F] | 1-[(4S)-2-(4-fluoro-3,5- dimethyl-phenyl)-4-methyl-5- [1-[(1S,2S)-2-methyl-1-(5- oxo-4H-1,2,4-oxadiazol-3- yl)cyclopropyl]-5- tetrahydropyran-4-yl-indole-2- carbonyl]-6,7-dihydro-4H- pyrazolo[4,3-c]pyridin-3-yl]- N-(1-methylindazol-5- yl)cyclopropanecarobxamide | 838.3 |
| 228 | 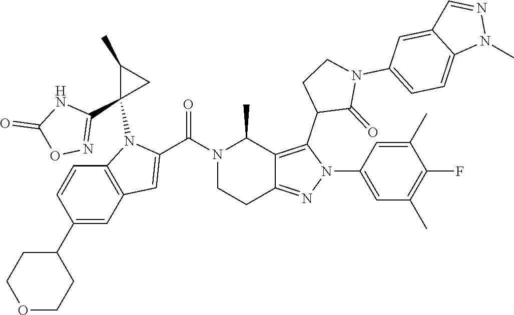 [SMILES: Cc1cc(-n2nc3c(c2C2CCN(c4ccc5c(cnn5C)c4)C2=O)[C@H](C)N(C(=O)c2cc4cc(C5CCOCC5)ccc4n2[C@@]2(c4noc(=O)[nH]4)C[C@@H]2C)CC3)cc(C)c1F] | 3-((1S,2S)-1-(2-((S)-2-(4- fluoro-3,5-dimethylphenyl)-4- methyl-3-((R)-1-(1-methyl- 1H-indazol-5-yl)-2- oxopyrrolidin-3-yl)-4,5,6,7 tetrahydro-2H-pyrazolo[4,3- c]pyridine-5-carbonyl)-5- (tetrahydro-2H-pyran-4-yl)- 1H-indol-1-yl)-2- methylcyclopropyl)-1,2,4- oxadiazol-5(2H)-one | 838.0 |

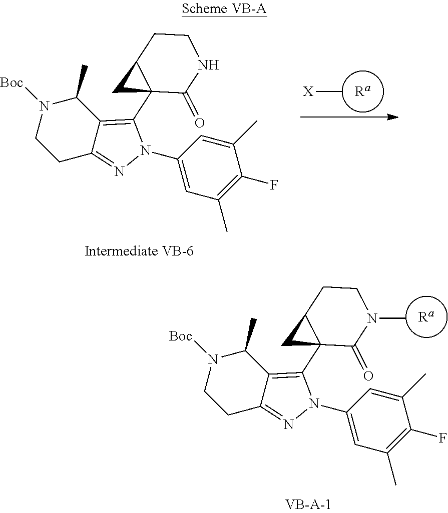

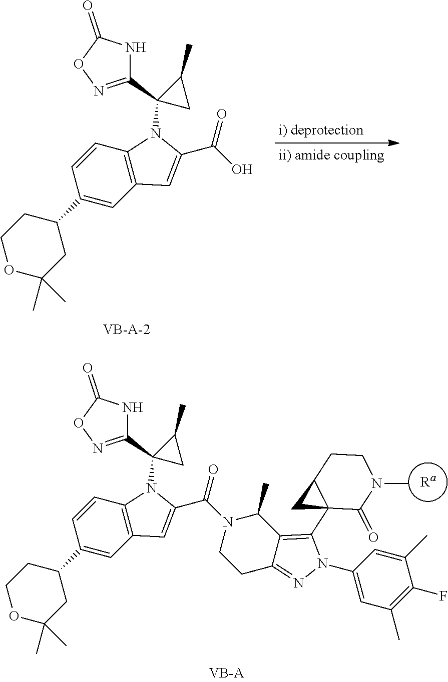 [SMILES: C[C@H]1C[C@]1(c1noc(=O)[nH]1)n1c(C(=O)O)cc2cc([C@H]3CCOC(C)(C)C3)ccc21 ; Cc1cc(-n2nc3c(c2[C@]24C[C@H]2CCN(C)C4=O)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F]

Compounds of formula VB-A are prepared from intermediate VB-6 by coupling with aryl halides via transition metal catalyzed cross-coupling conditions (e.g., Cu-catalysis) to provide VB-A-1. Removal of the Boc protecting group (e.g., acidic conditions) of VB-A-1 followed by coupling with carboxylic acid VB-A-2 provides compounds of formula VB-A.

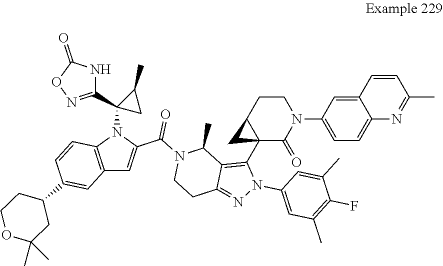 [SMILES: Cc1ccc2cc(N3CC[C@@H]4C[C@]4(c4c5c(nn4-c4cc(C)c(F)c(C)c4)CCN(C(=O)c4cc6cc([C@H]7CCOC(C)(C)C7)ccc6n4[C@@]4(c6noc(=O)[nH]6)C[C@@H]4C)[C@H]5C)C3=O)ccc2n1]

### 3-((1S,2S)-1-(5-((S)-2,2-dimethyltetrahydro-2H-pyran-4-yl)-2-((S)-2-(4-fluoro-3,5-dimethylphenyl)-4-methyl-3-((1S,6S)-3-(2-methylquinolin-6-yl)-2-oxo-3-azabicyclo[4.1.0]heptan-1-yl)-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one (Scheme VB-A)

### Step 1: tert-butyl (S)-2-(4-fluoro-3,5-dimethylphenyl)-4-methyl-3-((1S,6S)-3-(2-methylquinolin-6-yl)-2-oxo-3-azabicyclo[4.1.0]heptan-1-yl)-2,4,6,7-tetrahydro-5H-pyrazolo[4,3-c]pyridine-5-carboxylate

A mixture of tert-butyl (S)-2-(4-fluoro-3,5-dimethylphenyl)-4-methyl-3-((1S,6S)-2-oxo-3-azabicyclo[4.1.0]heptan-1-yl)-2,4,6,7-tetrahydro-5H-pyrazolo[4,3-c]pyridine-5-carboxylate (17 mg, 0.036 mmol), 6-bromo-2-methylquinoline (16 mg, 0.073 mmol), copper(I) iodide (6.9 mg, 0.036 mmol) and potassium phosphate (23 mg, 0.11 mmol) in toluene (0.36 mL) was sparged with argon for 5 min. After adding N,N′-dimethylethylenediamine (7.8 μL, 6.4 mg, 0.073 mmol), the reaction mixture was stirred under argon at 110° C. for 1 h. The mixture was cooled to rt and directly purified via silica gel column chromatography (0-100% (3:1 EtOAc/EtOH) in hexanes) to afford the title compound. MS (ESI): m/z 610.2 [M+H]+.

### Step 2: 3-((1S,2S)-1-(5-((S)-2,2-dimethyltetrahydro-2H-pyran-4-yl)-2-((S)-2-(4-fluoro-3,5-dimethylphenyl)-4-methyl-3-((1S,6S)-3-(2-methylquinolin-6-yl)-2-oxo-3-azabicyclo[4.1.0]heptan-1-yl)-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one

A mixture of tert-butyl (S)-2-(4-fluoro-3,5-dimethylphenyl)-4-methyl-3-((1S,6S)-3-(2-methylquinolin-6-yl)-2-oxo-3-azabicyclo[4.1.0]heptan-1-yl)-2,4,6,7-tetrahydro-5H-pyrazolo[4,3-c]pyridine-5-carboxylate (15 mg, 0.025 mmol) and 4M HCl in dioxane (0.19 mL, 0.74 mmol) was stirred at rt for 30 min. The reaction mixture was then concentrated. The residue was treated with 5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carboxylic acid (12 mg, 0.029 mmol), HATU (11 mg, 0.029 mmol) and DMF (0.16 mL). To the resultant mixture was added DIPEA (22 mg, 30 μL, 0.17 mmol). The reaction mixture was then stirred at rt overnight. The mixture was then syringe-filtered and purified via reverse phase prep-HPLC (40-70% MeCN/water+0.1% formic acid modifier) to provide the title compound. ¹H NMR (CD₃CN, 400 MHz, rotamers): δ 8.12 (1H, d, J=8.5 Hz), 7.92 (1H, d, J=8.9 Hz), 7.70 (1H, s), 7.60 (1H, d, J=8.9 Hz), 7.54-7.47 (2H, m), 7.37 (1H, d, J=8.5 Hz), 7.27 (1H, d, J=8.7 Hz), 7.15-7.07 (2H, m), 6.83 (1H, d, J=27.5 Hz), 5.71 (1H, dd, J=13.2, 6.3 Hz), 4.35 (1H, dd, J=14.1, 5.2 Hz), 3.80-3.45 (5H, m), 3.11-2.99 (2H, m), 2.92-2.82 (1H, m), 2.66 (3H, d, J=6.2 Hz), 2.34 (6H, d, J=13.7 Hz), 1.81-1.49 (13H, m), 1.27 (5H, d, J=7.6 Hz), 1.19-1.15 (6H, m). MS (ESI) m/z: 904.1 [M+H]+.

The following examples in Table 17 were prepared using the procedures outlined in the synthesis of Example 229.

TABLE 17

| | | | MS |
|---|---|---|---|
| Ex. | Structure | Name | (M + 1) |
| 230 | 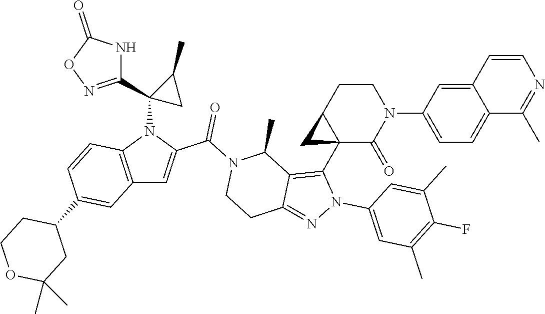 [SMILES: Cc1cc(-n2nc3c(c2[C@]24C[C@H]2CCN(c2ccc5c(C)nccc5c2)C4=O)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | 3-((1S,2S)-1-(5-((S)-2,2- dimethyltetrahydro-2H- pyran-4-yl)-2-((S)-2-(4- fluoro-3,5- dimethylphenyl)-4-methyl- 3-((1S,6S)-3-(1- methylisoquinolin-6-yl)-2- oxo-3- azabicyclo[4.1.0]heptan-1- yl)-4,5,6,7-tetrahydro-2H- pyrazolo[4,3-c]pyridine-5- carbonyl)-1H-indol-1-yl)-2- methylcyclopropyl)-1,2,4- oxadiazol-5(4H)-one | 904.1 |
| 231 | 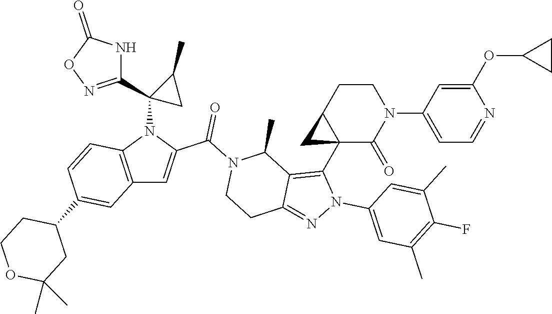 [SMILES: Cc1cc(-n2nc3c(c2[C@]24C[C@H]2CCN(c2ccnc(OC5CC5)c2)C4=O)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | 3-((1S,2S)-1-(2-((S)-3- ((1S,6S)-3-(2- cyclopropoxypyridin-4-yl)- 2-oxo-3- azabicyclo[4.1.0]heptan-1- yl)-2-(4-fluoro-3,5- dimethylphenyl)-4-methyl- 4,5,6,7-tetrahydro-2H- pyrazolo[4,3-c]pyridine-5- carbonyl)-5-((S)-2,2- dimethyltetrahydro-2H- pyran-4-yl)-1H-indol-1-yl)- 2-methylcyclopropyl)- 1,2,4-oxadiazol-5(4H)-one | 896.0 |
| 350 | 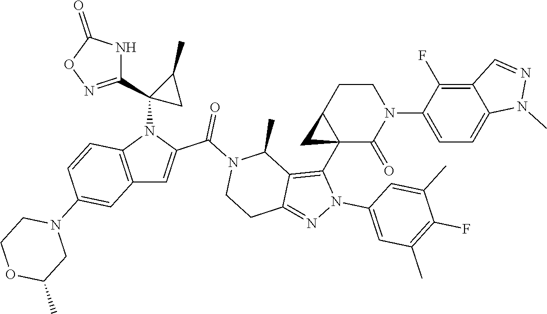 [SMILES: Cc1cc(-n2nc3c(c2[C@]24C[C@H]2CCN(c2ccc5c(cnn5C)c2F)C4=O)[C@H](C)N(C(=O)c2cc4cc(N5CCO[C@@H](C)C5)ccc4n2[C@@]2(c4noc(=O)[nH]4)C[C@@H]2C)CC3)cc(C)c1F] | 3-((1S,2S)-1-(2-((S)-3- ((1S,6S)-3-(4-fluoro-1- methyl-1H-indazol-5-yl)-2- oxo-3- azabicyclo[4.1.0]heptan-1- yl)-2-(4-fluoro-3,5- dimethylphenyl)-4-methyl- 4,5,6,7-tetrahydro-2H- pyrazolo[4,3-c]pyridine-5- carbonyl)-5-((S)-2- methylmorpholino)-1H- indol-1-yl)-2- methylcyclopropyl)-1,2,4- oxadiazol-5(4H)-one | 897.6 |
| 377 | 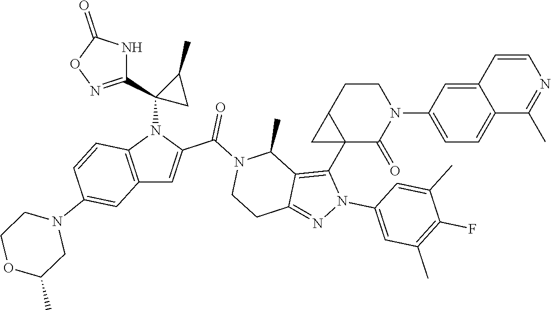 [SMILES: Cc1cc(-n2nc3c(c2C24CC2CCN(c2ccc5c(C)nccc5c2)C4=O)[C@H](C)N(C(=O)c2cc4cc(N5CCO[C@@H](C)C5)ccc4n2[C@@]2(c4noc(=O)[nH]4)C[C@@H]2C)CC3)cc(C)c1F] | 3-((1S,2S)-1-(2-((S)-2-(4- fluoro-3,5- dimethylphenyl)-4-methyl- 3-((1S,6S)-3-(1- methylisoquinolin-6-yl)-2- oxo-3- azabicyclo[4.1.0]heptan-1- yl)-4,5,6,7-tetrahydro-2H- pyrazolo[4,3-c]pyridine-5- carbonyl)-5-((S)-2- methylmorpholino)-1H- indol-1-yl)-2- methylcyclopropyl)-1,2,4- oxadiazol-5(4H)-one OR 3- ((1S,2S)-1-(2-((S)-2-(4- fluoro-3,5- dimethylphenyl)-4-methyl- 3-((1R,6R)-3-(1- methylisoquinolin-6-yl)-2- | 890.4 |
| | | oxo-3- | |
| | | azabicyclo[4.1.0]heptan-1- | |
| | | yl)-4,5,6,7-tetrahydro-2H- | |
| | | pyrazolo[4,3-c]pyridine-5- | |
| | | carbonyl)-5-((S)-2- | |
| | | methylmorpholino)-1H- | |
| | | indol-1-yl)-2- | |
| | | methylcyclopropyl)-1,2,4- | |
| | | oxadiazol-5(4H)-one | |
| 378 | 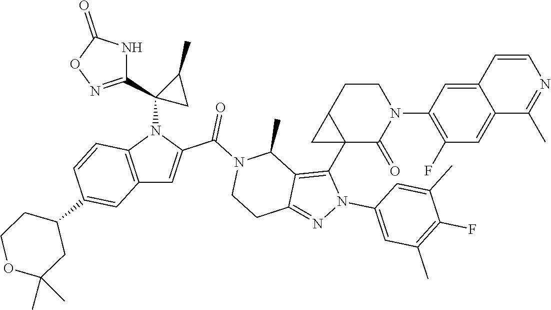 [SMILES: Cc1cc(-n2nc3c(c2C24CC2CCN(c2cc5ccnc(C)c5cc2F)C4=O)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | 3-((1S,2S)-1-(5-((S)-2,2- dimethyltetrahydro-2H- pyran-4-yl)-2-((S)-3- ((1R,6R)-3-(7-fluoro-1- methylisoquinolin-6-yl)-2- oxo-3- azabicyclo[4.1.0]heptan-1- yl)-2-(4-fluoro-3,5- dimethylphenyl)-4-methyl- 4,5,6,7-tetrahydro-2H- pyrazolo[4,3-c]pyridine-5- carbonyl)-1H-indol-1-yl)-2- methylcyclopropyl)-1,2,4- oxadiazol-5(4H)-one OR 3- ((1S,2S)-1-(5-((S)-2,2- dimethyltetrahydro-2H- pyran-4-yl)-2-((S)-3- ((1S,6S)-3-(7-fluoro-1- methylisoquinolin-6-yl)-2- | 921.3 |
| | | oxo-3- | |
| | | azabicyclo[4.1.0]heptan-1- | |
| | | yl)-2-(4-fluoro-3,5- | |
| | | dimethylphenyl)-4-methyl- | |
| | | 4,5,6,7-tetrahydro-2H- | |
| | | pyrazolo[4,3-c]pyridine-5- | |
| | | carbonyl)-1H-indol-1-yl)-2- | |
| | | methylcyclopropyl)-1,2,4- | |
| | | oxadiazol-5(4H)-one | |
| 379 | 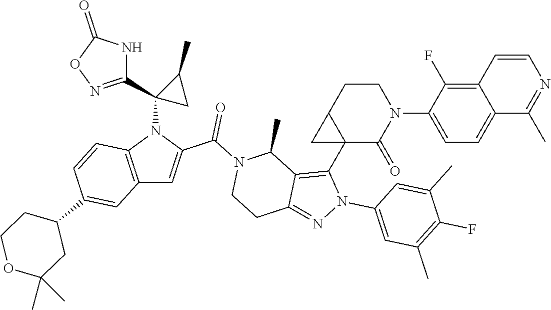 [SMILES: Cc1cc(-n2nc3c(c2C24CC2CCN(c2ccc5c(C)nccc5c2F)C4=O)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | 3-((1S,2S)-1-(5-((S)-2,2- dimethyltetrahydro-2H- pyran-4-yl)-2-((S)-3- ((1R,6R)-3-(5-fluoro-1- methylisoquinolin-6-yl)-2- oxo-3- azabicyclo[4.1.0]heptan-1- yl)-2-(4-fluoro-3,5- dimethylphenyl)-4-methyl- 4,5,6,7-tetrahydro-2H- pyrazolo[4,3-c]pyridine-5- carbonyl)-1H-indol-1-yl)-2- methylcyclopropyl)-1,2,4- oxadiazol-5(4H)-one OR 3- ((1S,2S)-1-(5-((S)-2,2- dimethyltetrahydro-2H- pyran-4-yl)-2-((S)-3- ((1S,6S)-3-(5-fluoro-1- methylisoquinolin-6-yl)-2- | 921.6 |
| | | oxo-3- | |
| | | azabicyclo[4.1.0]heptan-1- | |
| | | yl)-2-(4-fluoro-3,5- | |
| | | dimethylphenyl)-4-methyl- | |
| | | 4,5,6,7-tetrahydro-2H- | |
| | | pyrazolo[4,3-c]pyridine-5- | |
| | | carbonyl)-1H-indol-1-yl)-2- | |
| | | methylcyclopropyl)-1,2,4- | |
| | | oxadiazol-5(4H)-one | |
| 380 | 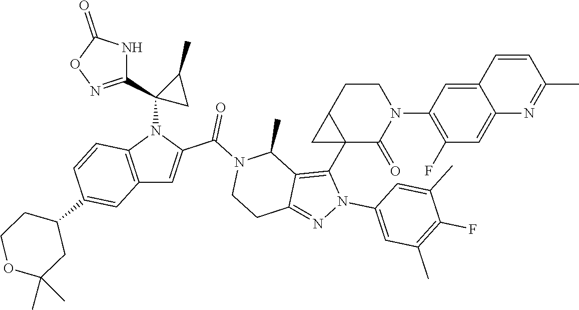 [SMILES: Cc1ccc2cc(N3CCC4CC4(c4c5c(nn4-c4cc(C)c(F)c(C)c4)CCN(C(=O)c4cc6cc([C@H]7CCOC(C)(C)C7)ccc6n4[C@@]4(c6noc(=O)[nH]6)C[C@@H]4C)[C@H]5C)C3=O)c(F)cc2n1] | 3-((1S,2S)-1-(5-((S)-2,2- dimethyltetrahydro-2H- pyran-4-yl)-2-((S)-3- ((1R,6R)-3-(7-fluoro-2- methylquinolin-6-yl)-2- oxo-3- azabicyclo[4.1.0]heptan-1- yl)-2-(4-fluoro-3,5- dimethylphenyl)-4-methyl- 4,5,6,7-tetrahydro-2H- pyrazolo[4,3-c]pyridine-5- carbonyl)-1H-indol-1-yl)-2- methylcyclopropyl)-1,2,4- oxadiazol-5(4H)-one OR 3- ((1S,2S)-1-(5-((S)-2,2- dimethyltetrahydro-2H- pyran-4-yl)-2-((S)-3- ((1S,6S)-3-(7-fluoro-2- methylquinolin-6-yl)-2- | 921.4 |
| | | oxo-3- | |
| | | azabicyclo[4.1.0]heptan-1- | |
| | | yl)-2-(4-fluoro-3,5- | |
| | | dimethylphenyl)-4-methyl- | |
| | | 4,5,6,7-tetrahydro-2H- | |
| | | pyrazolo[4,3-c]pyridine-5- | |
| | | carbonyl)-1H-indol-1-yl)-2- | |
| | | methylcyclopropyl)-1,2,4- | |
| | | oxadiazol-5(4H)-one | |
| 381 | 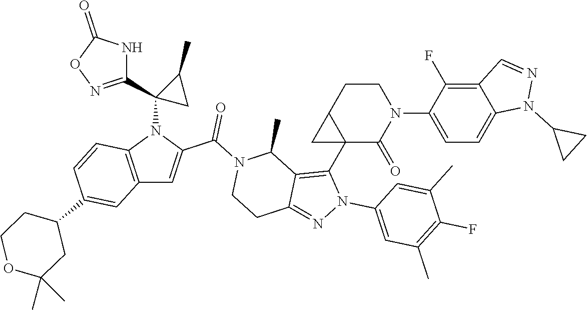 [SMILES: Cc1cc(-n2nc3c(c2C24CC2CCN(c2ccc5c(cnn5C5CC5)c2F)C4=O)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | 3-((1S,2S)-1-(2-((S)-3- ((1R,6R)-3-(1-cyclopropyl- 4-fluoro-1H-indazol-5-yl)- 2-oxo-3- azabicyclo[4.1.0]heptan-1- yl)-2-(4-fluoro-3,5- dimethylphenyl)-4-methyl- 4,5,6,7-tetrahydro-2H- pyrazolo[4,3-c]pyridine-5- carbonyl)-5-((S)-2,2- dimethyltetrahydro-2H- pyran-4-yl)-1H-indol-1-yl)- 2-methylcyclopropyl)- 1,2,4-oxadiazol-5(4H)-one OR 3-((1S,2S)-1-(2-((S)-3- ((1S,6S)-3-(1-cyclopropyl- 4-fluoro-1H-indazol-5-yl)- 2-oxo-3- azabicyclo[4.1.0]heptan-1- | 936.4 |
| | | yl)-2-(4-fluoro-3,5- | |
| | | dimethylphenyl)-4-methyl- | |
| | | 4,5,6,7-tetrahydro-2H- | |
| | | pyrazolo[4,3-c]pyridine-5- | |
| | | carbonyl)-5-((S)-2,2- | |
| | | dimethyltetrahydro-2H- | |
| | | pyran-4-yl)-1H-indol-1-yl)- | |
| | | 2-methylcyclopropyl)- | |
| | | 1,2,4-oxadiazol-5(4H)-one | |
| 382 | 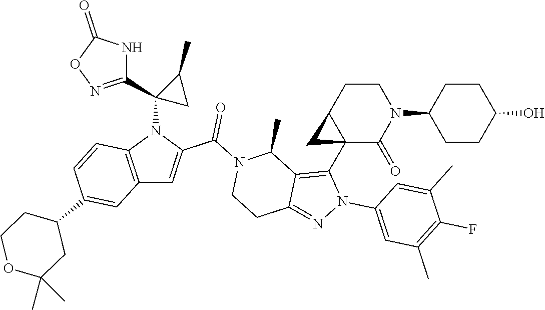 [SMILES: Cc1cc(-n2nc3c(c2[C@]24C[C@H]2CCN([C@H]2CC[C@H](O)CC2)C4=O)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | 3-((1S,2S)-1-(5-(2,2-(S)- dimethyltetrahydro-2H- pyran-4-yl)-2-((S)-2-(4- fluoro-3,5- dimethylphenyl)-3- ((1S,6S)-3-((1r,4S)-4- hydroxycyclohexyl)-2-oxo- 3-azabicyclo[4.1.0]heptan- 1-yl)-4-methyl-4,5,6,7- tetrahydro-2H- pyrazolo[4,3-c]pyridine-5- carbonyl)-1H-indol-1-yl)-2- methylcyclopropyl)-1,2,4- oxadiazol-5(4H)-one | 860.1 |
| 383 | 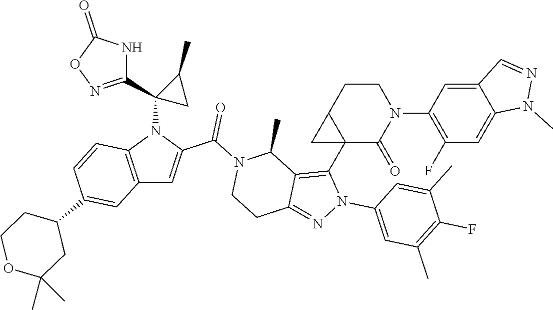 [SMILES: Cc1cc(-n2nc3c(c2C24CC2CCN(c2cc5cnn(C)c5cc2F)C4=O)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | 3-((1S,2S)-1-(5-((S)-2,2- dimethyltetrahydro-2H- pyran-4-yl)-2-((S)-3- ((1R,6R)-3-(6-fluoro-1- methyl-1H-indazol-5-yl)-2- oxo-3- azabicyclo[4.1.0]heptan-1- yl)-2-(4-fluoro-3,5- dimethylphenyl)-4-methyl- 4,5,6,7-tetrahydro-2H- pyrazolo[4,3-c]pyridine-5- carbonyl)-1H-indol-1-yl)-2- methylcyclopropyl)-1,2,4- oxadiazol-5(4H)-one OR 3- ((1S,2S)-1-(5-((S)-2,2- dimethyltetrahydro-2H- pyran-4-yl)-2-((S)-3- ((1S,6S)-3-(6-fluoro-1- methyl-1H-indazol-5-yl)-2- | 910.4 |
| | | oxo-3- | |
| | | azabicyclo[4.1.0]heptan-1- | |
| | | yl)-2-(4-fluoro-3,5- | |
| | | dimethylphenyl)-4-methyl- | |
| | | 4,5,6,7-tetrahydro-2H- | |
| | | pyrazolo[4,3-c]pyridine-5- | |
| | | carbonyl)-1H-indol-1-yl)-2- | |
| | | methylcyclopropyl)-1,2,4- | |
| | | oxadiazol-5(4H)-one | |
| 384 | 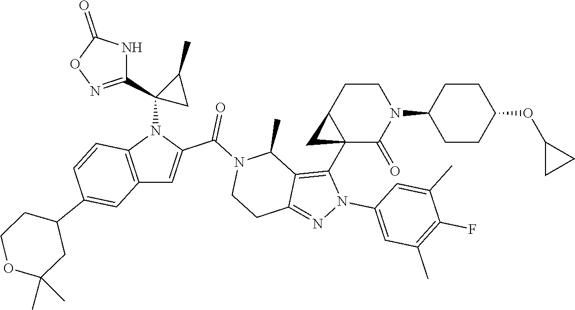 [SMILES: Cc1cc(-n2nc3c(c2[C@]24C[C@H]2CCN([C@H]2CC[C@H](OC5CC5)CC2)C4=O)[C@H](C)N(C(=O)c2cc4cc(C5CCOC(C)(C)C5)ccc4n2[C@@]2(c4noc(=O)[nH]4)C[C@@H]2C)CC3)cc(C)c1F] | 3-((1S,2S)-1-(5-(2,2- dimethyltetrahydro-2H- pyran-4-yl)-2-((S)-2-(4- fluoro-3,5- dimethylphenyl)-3- ((1S,6S)-3-((1r,4S)-4- hydroxycyclohexyl)-2-oxo- 3-azabicyclo[4.1.0]heptan- 1-yl)-4-methyl-4,5,6,7- tetrahydro-2H- pyrazolo[4,3-c]pyridine-5- carbonyl)-1H-indol-1-yl)-2- methylcyclopropyl)-1,2,4- oxadiazol-5(4H)-one | 900.6 |
| 422 | 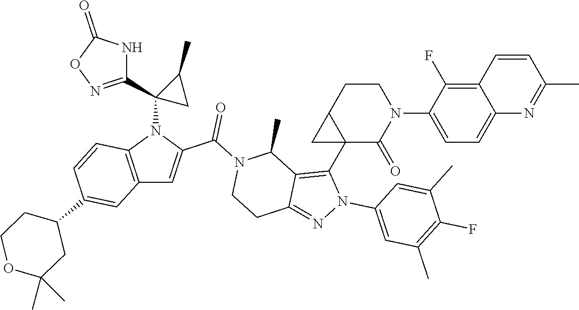 [SMILES: Cc1ccc2c(F)c(N3CCC4CC4(c4c5c(nn4-c4cc(C)c(F)c(C)c4)CCN(C(=O)c4cc6cc([C@H]7CCOC(C)(C)C7)ccc6n4[C@@]4(c6noc(=O)[nH]6)C[C@@H]4C)[C@H]5C)C3=O)ccc2n1] | 3-((1S,2S)-1-(5-((S)-2,2- dimethyltetrahydro-2H- pyran-4-yl)-2-((S)-3- ((1S,6S)-3-(5-fluoro-2- methylquinolin-6-yl)-2- oxo-3- azabicyclo[4.1.0]heptan-1- yl)-2-(4-fluoro-3,5- dimethylphenyl)-4-methyl- 4,5,6,7-tetrahydro-2H- pyrazolo[4,3-c]pyridine-5- carbonyl)-1H-indol-1-yl)-2- methylcyclopropyl)-1,2,4- oxadiazol-5(4H)-one OR 3- ((1S,2S)-1-(5-((S)-2,2- dimethyltetrahydro-2H- pyran-4-yl)-2-((S)-3- ((1R,6R)-3-(5-fluoro-2- methylquinolin-6-yl)-2- | 921.4 |
| | | oxo-3- | |
| | | azabicyclo[4.1.0]heptan-1- | |
| | | yl)-2-(4-fluoro-3,5- | |
| | | dimethylphenyl)-4-methyl- | |
| | | 4,5,6,7-tetrahydro-2H- | |
| | | pyrazolo[4,3-c]pyridine-5- | |
| | | carbonyl)-1H-indol-1-yl)-2- | |
| | | methylcyclopropyl)-1,2,4- | |
| | | oxadiazol-5(4H)-one | |

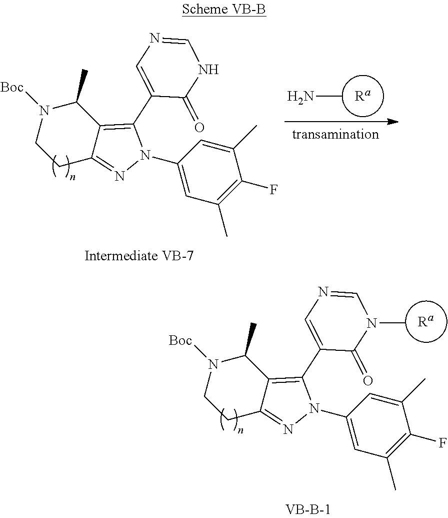

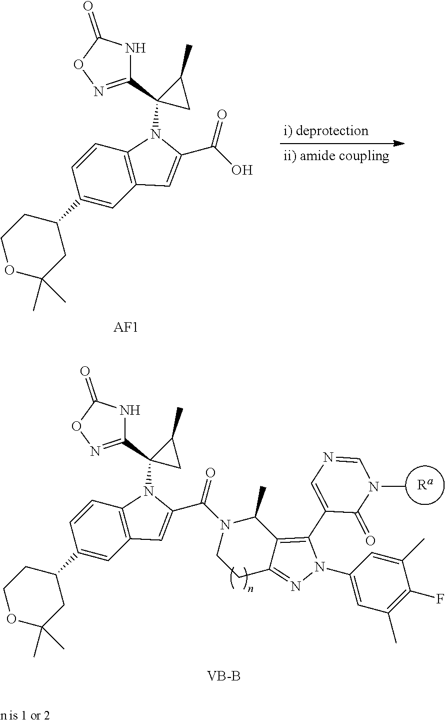

Compounds of formula VB—B are prepared from intermediate VB-7 by transamination with alkyl amines to provide VB—B-1. Removal of the Boc protecting group (e.g., acidic conditions) of VB—B-1 followed by coupling with carboxylic acid AF1 to provide compounds of formula VB—B.

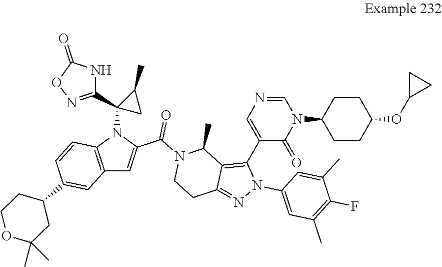 [SMILES: Cc1cc(-n2nc3c(c2-c2cncn([C@H]4CC[C@H](OC5CC5)CC4)c2=O)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F]

### 3-((1S,2S)-1-(2-((4S)-3-(1-((1r,4S)-4-cyclopropoxycyclohexyl)-6-oxo-1,6-dihydropyrimidin-5-yl)-2-(4-fluoro-3,5-dimethylphenyl)-4-methyl-4,5,6,7-tetrahydro-2H-pyrazolo[4,3-c]pyridine-5-carbonyl)-5-((S)-2,2-dimethyltetrahydro-2H-pyran-4-yl)-1H-indol-1-yl)-2-methylcyclopropyl)-1,2,4-oxadiazol-5(4H)-one (Scheme VB—B)

### Step 1: tert-butyl (4S)-3-(1-((1r,4S)-4-cyclopropoxycyclohexyl)-6-oxo-1,6-dihydropyrimidin-5-yl)-2-(4-fluoro-3,5-dimethylphenyl)-4-methyl-2,4,6,7-tetrahydro-5H-pyrazolo[4,3-c]pyridine-5-carboxylate

A mixture of tert-butyl (4S)-2-(4-fluoro-3,5-dimethylphenyl)-4-methyl-3-(6-oxo-1,6-dihydropyrimidin-5-yl)-2,4,6,7-tetrahydro-5H-pyrazolo[4,3-c]pyridine-5-carboxylate (43 mg, 0.094 mmol) and DIPEA (30 mg, 41 μL, 0.23 mmol) in DCE (3.1 mL) was treated with 2-chloro-1-methylpyridin-1-ium iodide (29 mg, 0.11 mmol). The reaction mixture was stirred at rt for 1 h. After adding (1r,4r)-4-cyclopropoxycyclohexan-1-amine (15 mg, 0.094 mmol) and additional DIPEA (30 mg, 41 μL, 0.23 mmol), the reaction mixture was stirred at 80° C. overnight. The mixture was concentrated and purified via silica gel column chromatography (0-100% (3:1 EtOAc/EtOH) in hexanes) to afford the title compound. MS (ESI): m/z 592.6 [M+H].

### Step 2: 3-((1S,2S)-1-(2-((4S)-3-(1-((1r,4S)-4-cyclopropoxycyclohexyl)-6-oxo-1,6-dihydropyrimidin-5-yl)-2-(4-fluoro-3,5-dimethylphenyl)-4-methyl-4,5,6,7-tetrahydro-2H-pyrazolo[4,3-c]pyridine-5-carbonyl)-5-((S)-2,2-dimethyltetrahydro-2H-pyran-4-yl)-1H-indol-1-yl)-2-methylcyclopropyl)-1,2,4-oxadiazol-5(4H)-one

A mixture of tert-butyl (4S)-3-(1-((1r,4S)-4-cyclopropoxycyclohexyl)-6-oxo-1,6-dihydropyrimidin-5-yl)-2-(4-fluoro-3,5-dimethylphenyl)-4-methyl-2,4,6,7-tetrahydro-5H-pyrazolo[4,3-c]pyridine-5-carboxylate (40 mg, 0.068 mmol) and 4M HCl in dioxane (0.26 mL, 1.0 mmol) was stirred at rt for 30 min. The reaction mixture was then concentrated. The residue was treated with 5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carboxylic acid (34 mg, 0.082 mmol), HATU (31 mg, 0.082 mmol) and DMF (0.34 mL). To the resultant mixture was added DIPEA (62 mg, 84 μL, 0.48 mmol). The reaction mixture was then stirred at rt for 3 h. The mixture was then directly purified via C18 reverse phase column chromatography (0-100% MeCN/water+0.1% formic acid modifier) to provide the title compound. ¹H NMR (500 MHz, CD₃CN) δ 11.39 (br s, 1H), 8.29 & 8.06 (rotameric s, 1H), 7.86-7.44 (m, 3H), 7.30-7.22 (m, 1H), 7.02 & 6.92 (rotameric d, J=6.3 Hz, 1H), 6.84 & 6.70 (rotameric s, 1H), 5.79 & 5.29 (rotameric q, J=6.7 Hz, 1H), 4.77 & 4.39 (rotameric dd, J=13-14, 5-6 Hz, 1H), 4.62-4.51 & 4.09-3.93 (rotameric m, 1H), 3.82-3.69 (m, 2H), 3.66-3.40 (m, 2H), 3.38-3.11 (m, 2H), 3.11-2.86 (m, 3H), 2.23-2.16 (m, 6H), 1.88-1.46 (m, 11H), 1.46-1.25 (m, 8H), 1.20 (s, 3H), 1.15-0.93 (m, 4H), 0.52-0.40 (m, 4H). MS (ESI) m/z: 885.4 [M+H]⁺.

The following examples in Table 18 were prepared using the procedures outlined in the synthesis of Example 232.

TABLE 18

| | | | MS |
|---|---|---|---|
| | | | (M + |
| Ex. | Structure | Name | 1) |
| 233 | 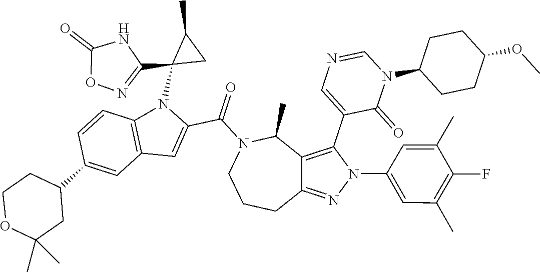 | 3-[(1S,2S)-1-[5-[(4S)-2,2- dimethyltetrahydropyran-4-yl]-2- [(4S)-2-(4-fluoro-3,5-dimethyl- phenyl)-3-[1-(4- methoxycyclohexyl)-6-oxo- pyrimidin-5-yl]-4-methyl-4,6,7,8- tetrahydropyrazolo[4,3-c]azepin- 1-ium-5-carbonyl]indol-1-yl]-2- methyl-cyclopropyl]-4H-1,2,4- oxadiazol-5-one formate | 873.8 |
| 337 | 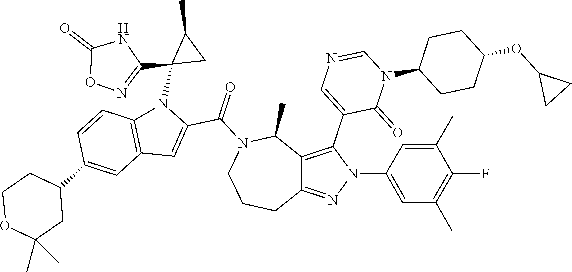 | 3-((1S,2S)-1-(2-((4S)-3-(1- ((1r,4S)-4- cyclopropoxycyclohexyl)-6-oxo- 1,6-dihydropyrimidin-5-yl)-2-(4- fluoro-3,5-dimethylphenyl)-4- methyl-2,4,5,6,7,8- hexahydropyrazolo[4,3- c]azepine-5-carbonyl)-5-((S)-2,2- dimethyltetrahydro-2H-pyran-4- yl)-1H-indol-1-yl)-2- methylcyclopropyl)-1,2,4- oxadiazol-5(4H)-one | 899.8 |

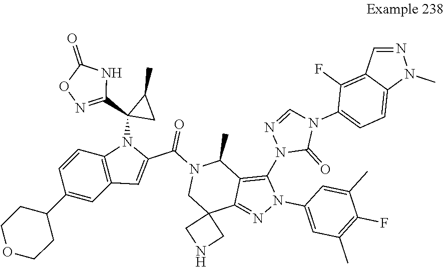 [SMILES: Cc1cc(-n2nc3c(c2-n2ncn(-c4ccc5c(cnn5C)c4F)c2=O)[C@H](C)N(C(=O)c2cc4cc(C5CCOCC5)ccc4n2[C@@]2(c4noc(=O)[nH]4)C[C@@H]2C)CC32CNC2)cc(C)c1F]

### 3-((1 S,2 S)-1-(2-((4′S)-3′-(3-(4-fluoro-1l-methyl-1H-indazol-5-yl)-2-oxo-2,3-dihydro-1H-imidazol-1-yl)-2′-(4-fluoro-3,5-dimethylphenyl)-4′-methyl-2′,4′,5′,6′-tetrahydrospiro[azetidine-3,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5 (4H)-one

### Step 1: 5′-benzyl 1-(tert-butyl) (S)-3′-(3-(2,2-dimethoxyethyl)-3-(4-fluoro-1-methyl-1H-indazol-5-yl)ureido)-2′-(4-fluoro-3,5-dimethylphenyl)-4′-methyl-2′,4′-dihydrospiro[azetidine-3,7′-pyrazolo[4,3-c]pyridine]-1,5′(6′H)-dicarboxylate

A solution of 5′-benzyl 1-(tert-butyl) (S)-3′-amino-2′-(4-fluoro-3,5-dimethylphenyl)-4′-methyl-2′,4′-dihydrospiro[azetidine-3,7′-pyrazolo[4,3-c]pyridine]-1,5′(6′H)-dicarboxylate (630 mg, 1.15 mmol) in THF (3.8 mL) was added dropwise to a solution of bis(trichloromethyl) carbonate (323 mg, 1.09 mmol) and DIPEA (1.04 g, 1.40 mL, 8.02 mmol) in THF (3.8 mL) kept at 0° C. in a flame-dried round-bottom flask. The reaction mixture was stirred at 0° C. for 2 h. Afterwards, a solution of N-(2,2-dimethoxyethyl)-4-fluoro-1-methyl-1H-indazol-5-amine (408 mg, 1.61 mmol) in THF (4.0 mL) was added dropwise at 0° C. The reaction mixture was slowly warmed to rt and stirred for 1 h. The mixture was then treated with 1M aqueous K₃PO₄ (20 mL) and EtOAc (20 mL). After mixing, the aqueous phase was extracted with EtOAc (2×20 mL). The combined organic layers were dried over MgSO₄, filtered, and concentrated under reduced pressure. The residue was purified via silica gel column chromatography (25-70% EtOAc/hexanes) to provide the title compound. MS (ESI) m/z: 829.7 [M+H]⁺.

### Step 2: 5′-benzyl 1-(tert-butyl) (4′S)-3′-(3-(4-fluoro-1-methyl-1H-indazol-5-yl)-2-oxo-2,3-dihydro-1H-imidazol-1-yl)-2′-(4-fluoro-3,5-dimethylphenyl)-4′-methyl-2′,4′-dihydrospiro[azetidine-3,7′-pyrazolo[4,3-c]pyridine]-1,5′(6′H)-dicarboxylate

A solution of 5′-benzyl 1-(tert-butyl) (S)-3′-(3-(2,2-dimethoxyethyl)-3-(4-fluoro-1-methyl-1H-indazol-5-yl)ureido)-2′-(4-fluoro-3,5-dimethylphenyl)-4′-methyl-2′,4′-dihydrospiro[azetidine-3,7′-pyrazolo[4,3-c]pyridine]-1,5′(6′H)-dicarboxylate (590 mg, 0.71 mmol) in dioxane (10 mL) was added to a solution of 4-methylbenzenesulfonic acid hydrate (149 mg, 0.78 mmol) in dioxane (25 mL) kept at 40° C. Mixture was stirred 40° C. for 18 h. Afterwards, 1M aqueous K₂CO₃ (30 mL), brine (30 mL) and EtOAc (30 mL) were added. After mixing, the aqueous phase was extracted with EtOAc (4×25 mL). The combined organic layers were washed with brine (25 mL), dried over MgSO₄, filtered, and concentrated under reduced pressure. The residue was purified via silica gel column chromatography (30-60% EtOAc/hexanes) to provide the title compound. MS (ESI) m/z: 665.6 [M−Boc+H]⁺.

### Step 3: tert-butyl (4′S)-3′-(3-(4-fluoro-1-methyl-1H-indazol-5-yl)-2-oxo-2,3-dihydro-1H-imidazol-1-yl)-2′-(4-fluoro-3,5-dimethylphenyl)-4′-methyl-2′,4′,5′,6′-tetrahydrospiro[azetidine-3,7′-pyrazolo[4,3-c]pyridine]-1-carboxylate

Triethylsilane (160 mg, 0.23 mL, 1.4 mmol) was added to a solution of 5′-benzyl 1-(tert-butyl) (4′S)-3′-(3-(4-fluoro-1-methyl-1H-indazol-5-yl)-2-oxo-2,3-dihydro-1H-imidazol-1-yl)-2′-(4-fluoro-3,5-dimethylphenyl)-4′-methyl-2′,4′-dihydrospiro[azetidine-3,7′-pyrazolo[4,3-c]pyridine]-1,5′(6′H)-dicarboxylate (270 mg, 0.35 mmol), triethylamine (39 mg, 54 μL, 0.39 mmol) and palladium(II) chloride (3.1 mg, 0.018 mmol) in THF (5.0 mL) under N₂. The mixture was stirred at rt for 1 h. The mixture was then diluted with MeCN (10 mL) and sequentially filtered through Celite® and a 45 μm syringe filter. The filtrate was diluted with toluene (40 mL) and concentrated under reduced pressure to yield the title compound, which was used in the next step without purification. MS (ESI) m/z: 631.6 [M+H]⁺.

### Step 4: tert-butyl (4′S)-3′-(3-(4-fluoro-1-methyl-1H-indazol-5-yl)-2-oxo-2,3-dihydro-1H-imidazol-1-yl)-2′-(4-fluoro-3,5-dimethylphenyl)-4′-methyl-5′-(1-((2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-2′,4′,5′,6′-tetrahydrospiro[azetidine-3,7′-pyrazolo[4,3-c]pyridine]-1-carboxylate

DIPEA (110 mg, 0.15 mL, 0.88 mmol) was added to a mixture of 1-((2S)-2-methyl-1-(5-oxo-4,5-dihydro-1,2,4-oxadiazol-3-yl)cyclopropyl)-5-(tetrahydro-2H-pyran-4-yl)-1H-indole-2-carboxylic acid (160 mg, 0.41 mmol), tert-butyl (4′S)-3′-(3-(4-fluoro-1-methyl-1H-indazol-5-yl)-2-oxo-2,3-dihydro-1H-imidazol-1-yl)-2′-(4-fluoro-3,5-dimethylphenyl)-4′-methyl-2′,4′,5′,6′-tetrahydrospiro[azetidine-3,7′-pyrazolo[4,3-c]pyridine]-1-carboxylate (370 mg, 50% wt, 0.29 mmol) and HATU (160 mg, 0.41 mmol) in DMA (7.4 mL). The reaction mixture was stirred at 60° C. for 1.5 h. The mixture was then diluted with EtOAc (40 mL), washed with brine (4×5 mL), dried over MgSO₄, filtered, and concentrated under reduced pressure. The residue was purified via silica gel column chromatography (50-90% EtOAc/hexanes) followed by purification via C18 reverse phase chromatography (30-95% MeCN/water) to provide the title compound. MS (ESI) m/z: 896.7 [M−Boc+H]⁺.

### Step 5: 3-((2S)-1-(2-((4′S)-3′-(3-(4-fluoro-1-methyl-1H-indazol-5-yl)-2-oxo-2,3-dihydro-1H-imidazol-1-yl)-2′-(4-fluoro-3,5-dimethylphenyl)-4′-methyl-2′,4′,5′,6′-tetrahydrospiro[azetidine-3,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one

Trifluoroacetic acid (2.2 g, 1.5 mL, 19 mmol) was added dropwise to a solution of tert-butyl (4′S)-3′-(3-(4-fluoro-1-methyl-1H-indazol-5-yl)-2-oxo-2,3-dihydro-1H-imidazol-1-yl)-2′-(4-fluoro-3,5-dimethylphenyl)-4′-methyl-5′-(1-((2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-2′,4′,5′,6′-tetrahydrospiro[azetidine-3,7′-pyrazolo[4,3-c]pyridine]-1-carboxylate (150 mg, 0.15 mmol) in DCM (1.5 mL) kept at 0° C. The mixture was stirred at 0° C. for 30 min. Afterwards, 1M aqueous K₃PO₄ (5 mL) was added, followed by saturated aqueous K₂CO₃ (until pH is over 9, ˜6 mL). The aqueous phase was filtered and extracted with EtOAc (4×10 mL). The combined organic layers were washed with 1M aqueous K₃PO₄ (5 mL) and brine (3×5 mL), combined with the solid residue from the previous filtration (redissolved with 2 mL MeCN), dried over MgSO₄, filtered, and concentrated under reduced pressure. The residue was purified via C18 reverse phase chromatography (30-95% MeCN/water with 10 mM ammonium bicarbonate modifier) to provide the title compound. ¹H NMR (400 MHz, CH₃CN-d₃, rotamers): δ_{H} 8.15 (0.5H, s), 8.05-8.07 (0.5H, m), 7.72 (0.5H, s), 7.59 (0.5H, d, J=8.6 Hz), 7.45-7.50 (2H, m), 7.15-7.36 (4H, m), 6.98-7.03 (1H, m), 6.84 (0.5H, br s), 6.72 (0.5H, s), 6.54-6.60 (1H, m), 6.33-6.47 (1H, m), 5.74 (0.5H, q, J=6.8 Hz), 5.29-5.54 (0.5H, m), 4.63-4.4.72 (1H, m), 4.20-4.36 (1H, m), 4.10 (2H, s), 4.04 (1H, s), 3.90-4.02 (3H, m), 3.29-3.61 (3H, m), 2.81-3.07 (2H, m), 2.53 (1H, s), 2.26-2.29 (5H, m), 2.19 (2H, s), 1.68-1.77 (4H, m), 1.47-1.60 (3H, m), 1.38 (3H, dd, J=6.5, 3.9 Hz), 1.30 (2H, s), 1.02 (1H, d, J=73.6 Hz). ¹⁹F NMR (376 MHz, CH₃CN-d₃, rotamers): δ_{F} −122.5-−122.9 (m; 1 F); −127.8-−127.9 (m, 1F). MS (ESI) m/z: 896.4 [M+H]⁺.

The following intermediates in Table 18.1 were prepared using the procedures outlined in the synthesis of intermediate VB-4.

TABLE 18.1

| | | | MS |
|---|---|---|---|
| Ex. | Structure | Name | (M + H) |
| 329 | 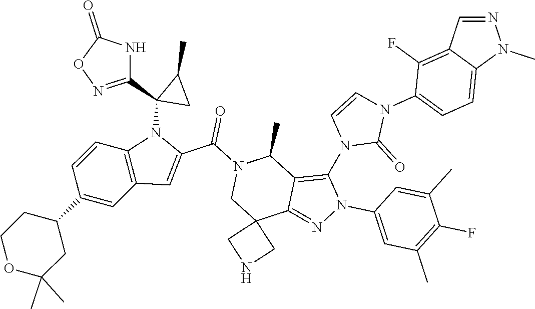 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)C[C@@H]2C)CC32CNC2)cc(C)c1F] | 3-((1S,2S)-1-(5-((S)-2,2- dimethyltetrahydro-2H-pyran-4- yl)-2-((4′S)-3′-(3-(4-fluoro-1- methyl-1H-indazol-5-yl)-2-oxo- 2,3-dihydro-1H-imidazol-1-yl)-2′- (4-fluoro-3,5-dimethylphenyl)-4′- methyl-2′,4′,5′,6′- tetrahydrospiro[azetidine-3,7′- pyrazolo[4,3-c]pyridine]-5′- carbonyl)-1H-indol-1-yl)-2- methylcyclopropyl)-1,2,4- oxadiazol-5(4H)-one | 924.6 |

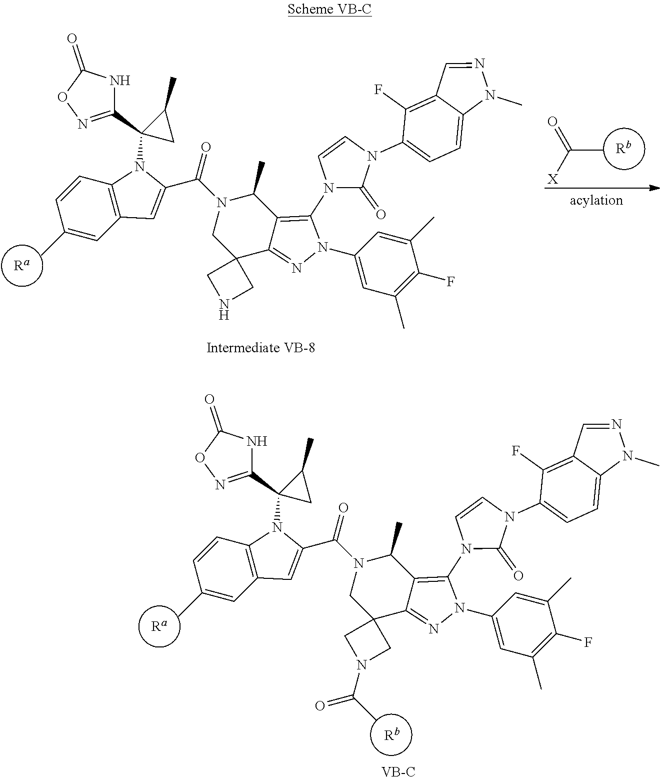

Compounds of formula VB—C are prepared from Intermediate VB-8 by acylation with an appropriate electrophile (e.g., acyl chloride, anhydride, etc.) to provide compounds of formula VB—C.

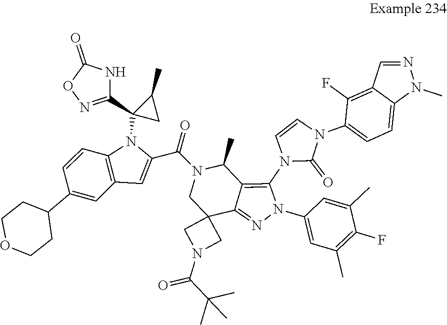

### 3-((1S,2S)-1-(2-((4′S)-3′-(3-(4-fluoro-1-methyl-1H-indazol-5-yl)-2-oxo-2,3-dihydro-1H-imidazol-1-yl)-2′-(4-fluoro-3,5-dimethylphenyl)-4′-methyl-1-pivaloyl-2′,4′,5′,6′-tetrahydrospiro[azetidine-3,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one (Scheme VB—C)

A mixture of 3-((1S,2S)-1-(2-((4′S)-3′-(3-(4-fluoro-1-methyl-1H-indazol-5-yl)-2-oxo-2,3-dihydro-1H-imidazol-1-yl)-2′-(4-fluoro-3,5-dimethylphenyl)-4′-methyl-2′,4′,5′,6′-tetrahydrospiro[azetidine-3,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one (6.2 mg, 0.0069 mmol) in DMF (0.07 mL) was sequentially treated with TEA (3.5 mg, 4.8 μL, 0.035 mmol) and pivaloyl chloride (1.3 mg, 1.3 μL, 0.0010 mmol). The mixture was stirred at rt for 30 min. The reaction mixture was then directly purified via C18 reverse phase column chromatography (10-100% MeCN/water+0.1% formic acid modifier) to provide the title compound. ¹H NMR (500 MHz, CD₃CN) δ 10.74 (br s, 1H), 8.18-7.97 (m, 1H), 7.62-7.07 (m, 7H), 6.93-6.32 (m, 3H), 5.70-5.30 (m, 1H), 5.22-5.09 (m, 1H), 4.98 (br m, 1H), 4.45 (br s, 2H), 4.14-3.91 (m, 6H), 3.78-3.44 (m, 3H), 2.93-2.81 (m, 1H), 2.34-2.24 (m, 6H), 1.79-1.39 (m, 9H), 1.37-1.02 (m, 13H). MS (ESI) m/z: 980.4 [M+H]⁺.

The following examples in Table 19 were prepared using the procedures outlined in the synthesis of Example 234.

TABLE 19

| | | | MS |
|---|---|---|---|
| Ex. | Structure | Name | (M + 1) |
| 235 | 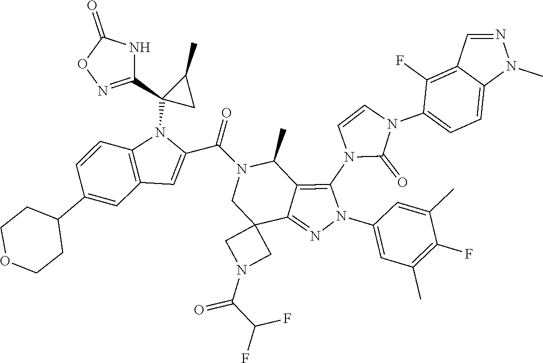 | 3-((1S,2S)-1-(2-((4′S)-1- (2,2-difluoroacetyl)-3′-(3- (4-fluoro-1-methyl-1H- indazol-5-yl)-2-oxo-2,3- dihydro-1H-imidazol-1- yl)-2′-(4-fluoro-3,5- dimethylphenyl)-4'- methyl-2′,4′,5′,6′- tetrahydrospiro[azetidine- 3,7′-pyrazolo[4,3- c]pyridine]-5′-carbonyl)-5- (tetrahydro-2H-pyran-4- yl)-1H-indol-1-yl)-2- methylcyclopropyl)-1,2,4- oxadiazol-5(4H)-one | 974.4 |
| 236 | 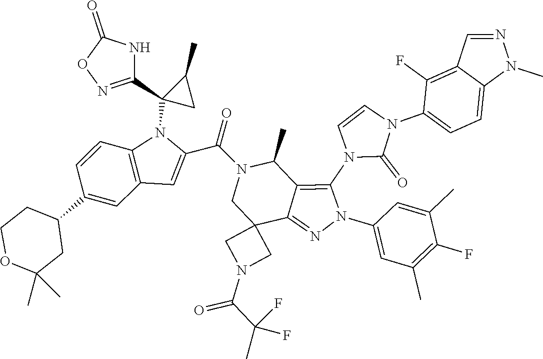 | 3-((1S,2S)-1-(2-((4′S)-1- (2,2-difluoropropanoyl)-3′- (3-(4-fluoro-1-methyl-1H- indazol-5-yl)-2-oxo-2,3- dihydro-1H-imidazol-1- yl)-2′-(4-fluoro-3,5- dimethylphenyl)-4′- methyl-2′,4′,5′,6′- tetrahydrospiro[azetidine- 3,7′-pyrazolo[4,3- c]pyridine]-5′-carbonyl)-5- ((S)-2,2- dimethyltetrahydro-2H- pyran-4-yl)-1H-indol-1- yl)-2-methylcyclopropyl)- 1,2,4-oxadiazol-5(4H)-one | 1029.0 (M + Na) |
| 237 | 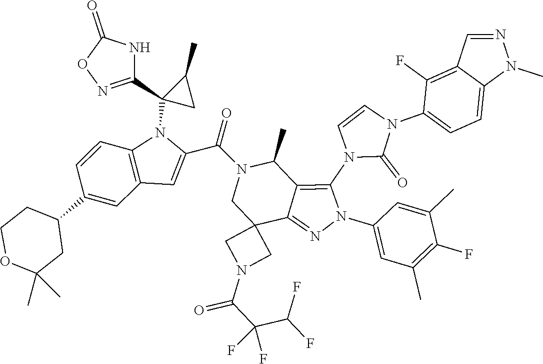 | 3-((1S,2S)-1-(5-((S)-2,2- dimethyltetrahydro-2H- pyran-4-yl)-2-((4′S)-3′-(3- (4-fluoro-1-methyl-1H- indazol-5-yl)-2-oxo-2,3- dihydro-1H-imidazol-1- yl)-2′-(4-fluoro-3,5- dimethylphenyl)-4′- methyl-1-(2,2,3,3- tetrafluoropropanoyl)- 2′,4′,5′,6′- tetrahydrospiro[azetidine- 3,7′-pyrazolo[4,3- c]pyridine]-5′-carbonyl)- 1H-indol-1-y1)-2- methylcyclopropyl)-1,2,4- oxadiazol-5(4H)-one | 1052.4 |
| 238 | 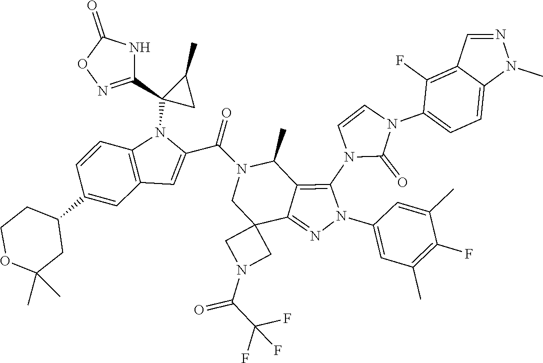 | 3-((1S,2S)-1-(5-((S)-2,2- dimethyltetrahydro-2H- pyran-4-yl)-2-((4′S)-3′-(3- (4-fluoro-1-methyl-1H- indazol-5-yl)-2-oxo-2,3- dihydro-1H-imidazol-1- yl)-2′-(4-fluoro-3,5- dimethylphenyl)-4′- methyl-1-(2,2,2- trifluoroacetyl)-2′,4′,5′,6′- tetrahydrospiro[azetidine- 3,7′-pyrazolo[4,3- c]pyridine]-5′-carbonyl)- 1H-indol-1-yl)-2- methylcyclopropyl)-1,2,4- oxadiazol-5(4H)-one | 1020.2 |
| 239 | 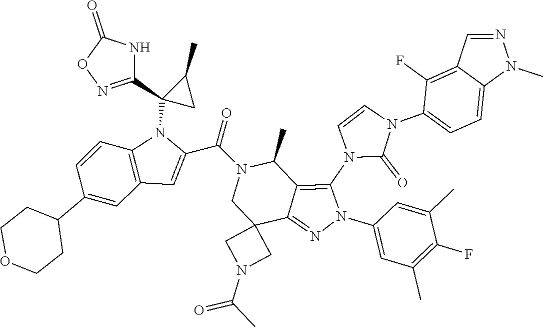 | 3-((1S,2S)-1-(2-((4′S)-1- acetyl-3′-(3-(4-fluoro-1- methyl-1H-indazol-5-yl)- 2-oxo-2,3-dihydro-1H- imidazol-1-yl)-2′-(4- fluoro-3,5- dimethylphenyl)-4′- methyl-2′,4′,5′,6′- tetrahydrospiro[azetidine- 3,7′-pyrazolo[4,3- c]pyridine]-5′-carbonyl)-5- (tetrahydro-2H-pyran-4- yl)-1H-indol-1-yl)-2- methylcyclopropyl)-1,2,4- oxadiazol-5(4H)-one | 938.8 |

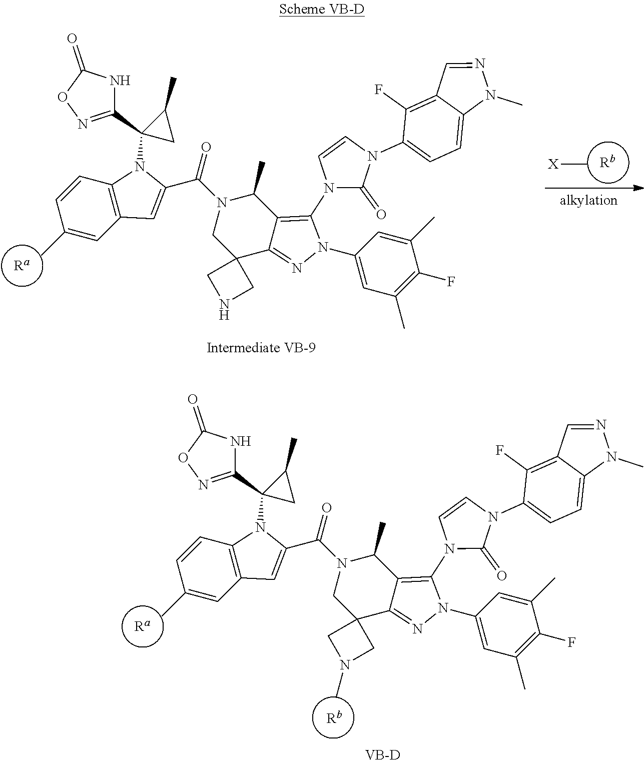

Compounds of formula VB-D are prepared from Intermediate VB-9 by alkylation with an appropriate electrophile (e.g., bromide, triflate etc.) to provide compounds of formula VB-D.

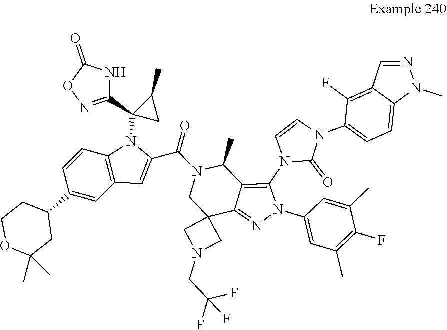

### 3-((1S,2S)-1-(5-((S)-2,2-dimethyltetrahydro-2H-pyran-4-yl)-2-((4′S)-3′-(3-(4-fluoro-1-methyl-1H-indazol-5-yl)-2-oxo-2,3-dihydro-1H-imidazol-1-yl)-2′-(4-fluoro-3,5-dimethylphenyl)-4′-methyl-1-(2,2,2-trifluoroethyl)-2′,4′,5′,6′-tetrahydrospiro[azetidine-3,7′-pyrazolo[4,3-c]pyridine]-5′-carbonyl)-1H-indol-1-yl)-2-methylcyclopropyl)-1,2,4-oxadiazol-5(4H)-one (Scheme VB-D)

A mixture of 3-((1S,2S)-1-(5-((S)-2,2-dimethyltetrahydro-2H-pyran-4-yl)-2-((4′S)-3′-(3-(4-fluoro-1-methyl-1H-indazol-5-yl)-2-oxo-2,3-dihydro-1H-imidazol-1-yl)-2′-(4-fluoro-3,5-dimethylphenyl)-4′-methyl-2′,4′,5′,6′-tetrahydrospiro[azetidine-3,7′-pyrazolo[4,3-c]pyridine]-5′-carbonyl)-1H-indol-1-yl)-2-methylcyclopropyl)-1,2,4-oxadiazol-5(4H)-one 2,2,2-trifluoroacetate (20 mg, 0.019 mmol), TEA (9.7 mg, 10 μL, 0.071 mmol) and 2,2,2-trifluoroethyl trifluoromethylsulfonate (4.4 mg, 2.8 μL, 0.019 mmol) in DMF (0.2 mL) was stirred at rt for 2 h. After adding additional 2,2,2-trifluoroethyl trifluoromethylsulfonate (3.3 mg, 2.8 μL, 0.014 mmol), the mixture was stirred at rt for another 2.5 h. Afterwards, the reaction mixture was directly purified via C18 reverse phase column chromatography (10-100% MeCN/water+0.1% TFA modifier) to provide the title compound as the TFA salt. ¹H NMR (500 MHz, CD₃CN) δ 11.27 (s, 1H), 8.14-7.97 (m, 1H), 7.60-7.34 (m, 3H), 7.32-7.09 (m, 4H), 6.90-6.81 (m, 1H), 6.77-6.54 (m, 1H), 6.55-6.32 (m, 1H), 5.70-4.79 (m, 2H), 4.20-3.93 (m, 5H), 3.83-3.59 (m, 4H), 3.50-3.00 (m, 5H), 2.33-2.26 (m, 6H), 1.77-1.39 (m, 9H), 1.33-0.94 (m, 9H). ¹⁹F NMR (471 MHz, CD₃CN) δ −71.48-−71.62 (m, 3F), −122.93 (s, 1F), −127.76-−127.89 (m, 1F). MS (ESI) m/z: 1006.4 [M+H]⁺.

The following Examples in Table 20 were prepared using the procedures outlined in the synthesis of Example 240.

TABLE 20

| | | | MS |
|---|---|---|---|
| Ex. | Structure | Name | (M + 1) |
| 241 | 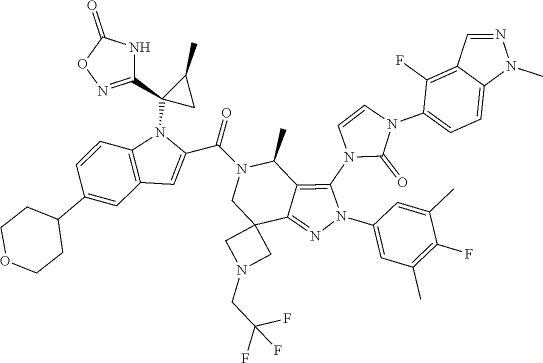 | 3-((1S,2S)-1-(2-((4′S)-3′- (3-(4-fluoro-1-methyl-1H- indazol-5-yl)-2-oxo-2,3- dihydro-1H-imidazol-1- yl)-2′-(4-fluoro-3,5- dimethylphenyl)-4′- methyl-1-(2,2,2- trifluoroethyl)-2′,4′,5′,6′- tetrahydrospiro[azetidine- 3,7′-pyrazolo[4,3- c]pyridine]-5′-carbonyl)-5- (tetrahydro-2H-pyran-4- yl)-1H-indol-1-yl)-2- methylcyclopropyl)-1,2,4- oxadiazol-5(4H)-one | 978.4 |
| 242 | 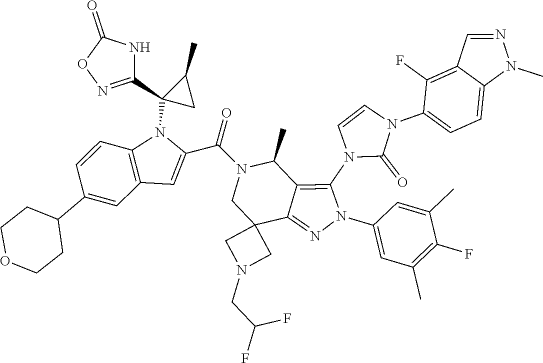 | 3-((1S,2S)-1-(2-((4′S)-1- (2,2-difluoroethyl)-3′-(3- (4-fluoro-1-methyl-1H- indazol-5-yl)-2-oxo-2,3- dihydro-1H-imidazol-1- yl)-2′-(4-fluoro-3,5- dimethylphenyl)-4′- methyl-2',4',5',6'- tetrahydrospiro[azetidine- 3,7'-pyrazolo [4,3- c]pyridine]-5'-carbonyl)-5- (tetrahydro-2H-pyran-4- yl)-1H-indol-1-yl)-2- methylcyclopropyl)-1,2,4- oxadiazol-5(4H)-one | 960.6 |
| 243 | 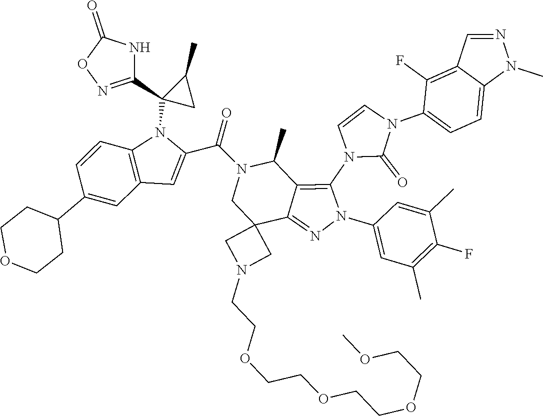 | 3-((1S,2S)-1-(2-((4′S)-3′- (3-(4-fluoro-1-methyl-1H- indazol-5-yl)-2-oxo-2,3- dihydro-1H-imidazol-1- yl)-2′-(4-fluoro-3,5- dimethylphenyl)-4′- methyl-1-(2,5,8,11- tetraoxatridecan-13-yl)- 2′,4′,5′,6′- tetrahydrospiro[azetidine- 3,7′-pyrazolo[4,3- c]pyridine]-5′-carbonyl)-5- (tetrahydro-2H-pyran-4- yl)-1H-indol-1-yl)-2- methylcyclopropyl)-1,2,4- oxadiazol-5(4H)-one | 1086.4 |
| 244 | 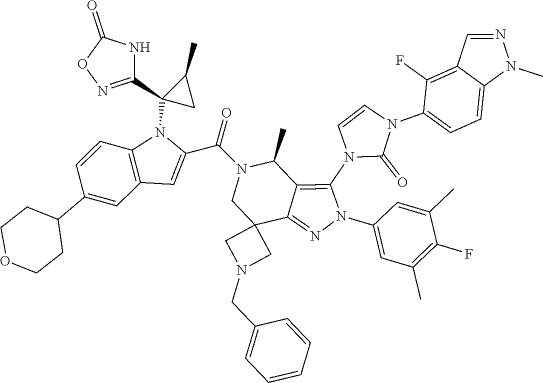 | 3-((1S,2S)-1-(2-((4′S)-1- benzyl-3′-(3-(4-fluoro-1- methyl-1H-indazol-5-yl)- 2-oxo-2,3-dihydro-1H- imidazol-1-yl)-2′-(4- fluoro-3,5- dimethylphenyl)-4′- methyl-2′,4′,5′,6′- tetrahydrospiro[azetidine- 3,7′-pyrazolo[4,3- c]pyridine]-5′-carbonyl)-5- (tetrahydro-2H-pyran-4- yl)-1H-indol-1-yl)-2- methylcyclopropyl)-1,2,4- oxadiazol-5(4H)-one | 986.6 |

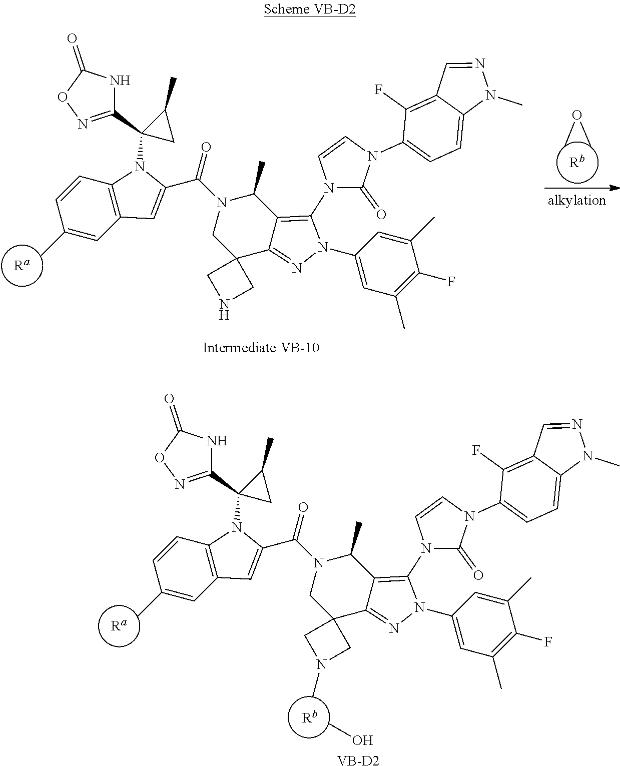

Compounds of formula VB-D2 are prepared from Intermediate VB-10 by alkylation with an appropriate epoxide to provide compounds of formula VB-D2.

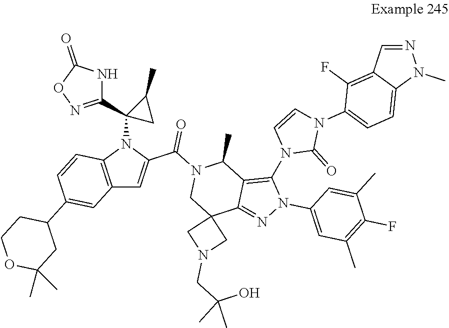 [SMILES: Cc1cc(-n2nc3c(c2-n2ccn(-c4ccc5c(cnn5C)c4F)c2=O)[C@H](C)N(C(=O)c2cc4cc(C5CCOC(C)(C)C5)ccc4n2[C@@]2(c4noc(=O)[nH]4)C[C@@H]2C)CC32CN(CC(C)(C)O)C2)cc(C)c1F]

### 3-((1S,2S)-1-(5-((S)-2,2-dimethyltetrahydro-2H-pyran-4-yl)-2-((4′S)-3′-(3-(4-fluoro-1-methyl-1H-indazol-5-yl)-2-oxo-2,3-dihydro-1H-imidazol-1-yl)-2′-(4-fluoro-3,5-dimethylphenyl)-1-(2-hydroxy-2-methylpropyl)-4′-methyl-2′,4′,5′,6′-tetrahydrospiro[azetidine-3,7′-pyrazolo[4,3-c]pyridine]-5′-carbonyl)-1H-indol-1-yl)-2-methylcyclopropyl)-1,2,4-oxadiazol-5(4H)-one (Scheme VB-D2)

A mixture of 3-((1S,2S)-1-(5-((S)-2,2-dimethyltetrahydro-2H-pyran-4-yl)-2-((4′S)-3′-(3-(4-fluoro-1-methyl-1H-indazol-5-yl)-2-oxo-2,3-dihydro-1H-imidazol-1-yl)-2′-(4-fluoro-3,5-dimethylphenyl)-4′-methyl-2′,4′,5′,6′-tetrahydrospiro[azetidine-3,7′-pyrazolo[4,3-c]pyridine]-5′-carbonyl)-1H-indol-1-yl)-2-methylcyclopropyl)-1,2,4-oxadiazol-5(4H)-one hydrochloride (5.1 mg, 0.0053 mmol), TEA (1.6 mg, 2.2 μL, 0.071 mmol) and 1,2-epoxy-isobutane (0.4 mg, 0.5 μL, 0.005 mmol) was stirred at 80° C. for 45 min. After adding additional and 1,2-epoxy-isobutane (0.4 mg, 0.5 μL, 0.005 mmol), the mixture was stirred at 80° C. for 20 min. Afterwards, 1,2-epoxy-isobutane (0.8 mg, 0.8 μL, 0.01 mmol) was added and the mixture was stirred at 60° C. overnight. After adding additional and 1,2-epoxy-isobutane (0.4 mg, 0.5 μL, 0.005 mmol), the mixture was stirred at 60° C. for 1 h, followed by stirring at 80° C. for 45 min. The reaction mixture was then directly purified via C18 reverse phase column chromatography (10-100% MeCN/water+0.1% TFA modifier) to provide the title compound as the TFA salt. ¹H NMR (500 MHz, CD₃CN) δ 8.17-7.99 (m, 1H), 7.63-7.09 (m, 7H), 6.95-6.27 (m, 3H), 5.54-4.55 (m, 7H), 4.04 (s, 3H), 3.84-3.45 (m, 3H), 3.43-3.00 (m, 2H), 2.28 (s, 6H), 1.77-1.38 (m, 1OH), 1.37-1.21 (m, 11H), 1.19 (s, 3H), 1.04-0.81 (m, 3H). MS (ESI) m/z: 996.5 [M+H]⁺.

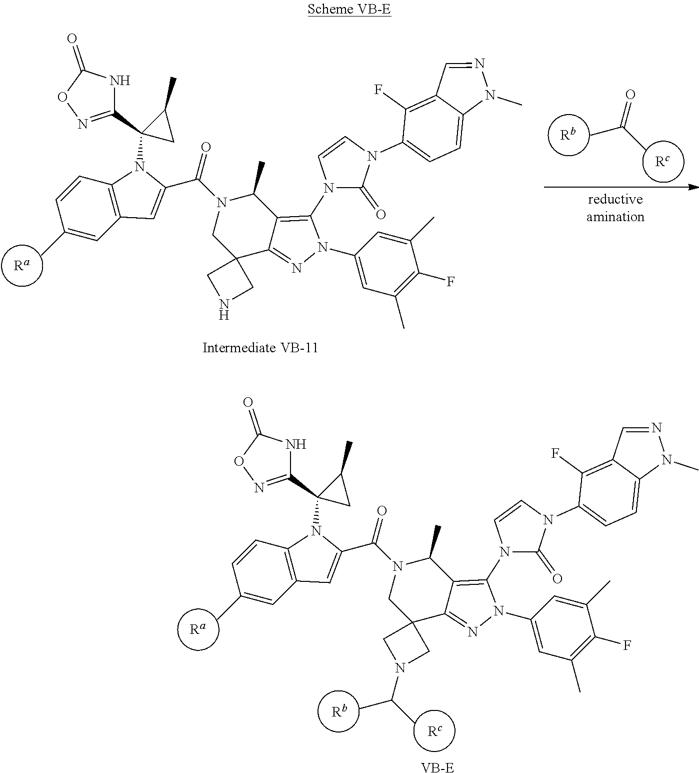

Compounds of formula VB-E are prepared from Intermediate VB-11 by reductive amination with aldehydes or ketones to provide compounds of formula (VB-E).

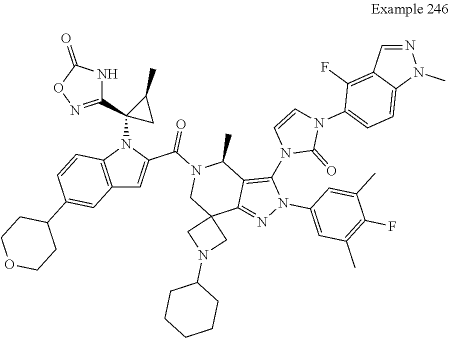 [SMILES: Cc1cc(-n2nc3c(c2-n2ccn(-c4ccc5c(cnn5C)c4F)c2=O)[C@H](C)N(C(=O)c2cc4cc(C5CCOCC5)ccc4n2[C@@]2(c4noc(=O)[nH]4)C[C@@H]2C)CC32CN(C3CCCCC3)C2)cc(C)c1F]

### 3-((1S,2S)-1-(2-((4′S)-1-cyclohexyl-3′-(3-(4-fluoro-1-methyl-1H-indazol-5-yl)-2-oxo-2,3-dihydro-1H-imidazol-1-yl)-2′-(4-fluoro-3,5-dimethylphenyl)-4′-methyl-2′,4′,5′,6′-tetrahydrospiro[azetidine-3,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one (Scheme VB-E)

A mixture of 3-((1S,2S)-1-(2-((4′S)-3′-(3-(4-fluoro-1-methyl-1H-indazol-5-yl)-2-oxo-2,3-dihydro-1H-imidazol-1-yl)-2′-(4-fluoro-3,5-dimethylphenyl)-4′-methyl-2′,4′,5′,6′-tetrahydrospiro[azetidine-3,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one (5.0 mg, 0.0056 mmol), cyclohexanone (1.6 mg, 1.7 μL, 0.017 mmol) and acetic acid (0.67 mg, 0.64 μL, 0.011 mmol) in DCE (0.06 mL) was stirred at rt for 5 min. After adding sodium triacetoxyborohydride (3.5 mg, 0.017 mmol), the reaction mixture was stirred at rt for 1 h. The mixture was then directly purified via C18 reverse-phase column chromatography (10-100% MeCN/water+0.100 TFA) to provide the title compound as the TFA salt. ¹H NMR (500 MHz, CD₃CN) δ 12.40 (br s, 1H), 11.04 (br s, 1H), 8.18-7.95 (m, 1H), 7.64-7.10 (m, 7H), 6.97-6.29 (m, 3H), 5.71-5.20 (m, 2H), 5.15-4.71 (m, 1H), 4.71-4.39 (m, 2H), 4.18-3.91 (m, 6H), 3.69 (d, J=13.1 Hz, 1H), 3.58-3.42 (m, 2H), 3.39-2.80 (m, 2H), 2.29 (s, 6H), 1.79-1.53 (m, 10H), 1.51-1.13 (m, 9H), 1.09-0.83 (in, 3H). MS (ESI) m/z: 978.4 [M+H]⁺.

The following example(s) in Table 21 were prepared using the procedures outlined in the synthesis of Example 246.

TABLE 21

| | | | MS |
|---|---|---|---|
| Ex | Structure | Name | (M + 1) |
| 247 | 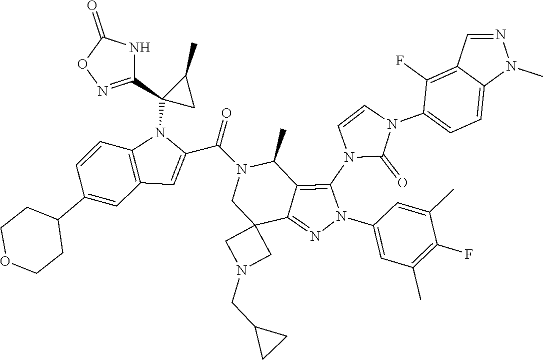 | 3-((1S,2S)-1-(2-((4′S)-1- (cyclopropylmethyl)-3′-(3- (4-fluoro-1-methyl-1H- indazol-5-yl)-2-oxo-2,3- dihydro-1H-imidazol-1- yl)-2′-(4-fluoro-3,5- dimethylphenyl)-4′- methyl-2′,4′,5′,6′- tetrahydrospiro[azetidine- 3,7′-pyrazolo[4,3- c]pyridine]-5′-carbonyl)-5- (tetrahydro-2H-pyran-4- yl)-1H-indol-1-yl)-2- methylcyclopropyl)-1,2,4- oxadiazol-5(4H)-one | 950.6 |

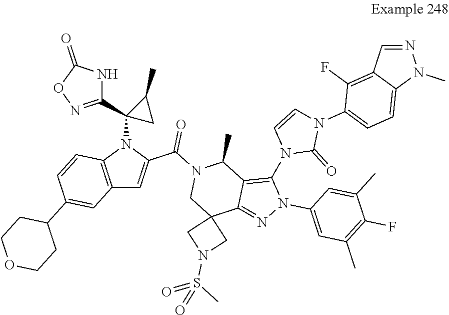 [SMILES: Cc1cc(-n2nc3c(c2-n2ccn(-c4ccc5c(cnn5C)c4F)c2=O)[C@H](C)N(C(=O)c2cc4cc(C5CCOCC5)ccc4n2[C@@]2(c4noc(=O)[nH]4)C[C@@H]2C)CC32CN(S(C)(=O)=O)C2)cc(C)c1F]

### 3-((1S,2S)-1-(2-((4′S)-3′-(3-(4-fluoro-1-methyl-1H-indazol-5-yl)-2-oxo-2,3-dihydro-1H-imidazol-1-yl)-2′-(4-fluoro-3,5-dimethylphenyl)-4′-methyl-1-(methylsulfonyl)-2′,4′,5′,6′-tetrahydrospiro[azetidine-3,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one

Methanesulfonic anhydride (2.3 mg, 13 mol) was added to a mixture of 3-((2S)-1-(2-((4′S)-3′-(3-(4-fluoro-1-methyl-1H-indazol-5-yl)-2-oxo-2,3-dihydro-1H-imidazol-1-yl)-2′-(4-fluoro-3,5-dimethylphenyl)-4′-methyl-2′,4′,5′,6′-tetrahydrospiro[azetidine-3,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one (10 mg, 11 mol) and pyridine (1.1 mg, 1.2 μL, 15 mol) in DCM (100 L) at room temperature in a flame-dried 2 mL conical vial. The mixture was stirred at room temperature for 2 h, then quenched with sat aqueous NaHCO₃ (0.5 mL) followed by addition of 0.5 mL DMSO. The mixture was concentrated under reduced pressure and directly loaded on a C18 column (12 g Biotage, liquid loading from DMSO) and eluted with a flow of 40-80% MeCN/H₂O (12 mL/min, 20 column volumes). ¹H NMR (400 MHz, CD₃CN): δ 8.07 (d; J=8.64 Hz; 1 H); 7.42-7.54 (m; 2.5 H); 7.17-7.35 (m; 5 H); 6.77-6.89 (m; 0.5 H); 6.60 (s; 0.5 H); 6.55 (s; 0.5 H); 6.46 (d; J=3.14 Hz; 0.5 H); 6.36 (d; J=3.37 Hz; 0.5 H); 5.68 (br s, 0.5H); 5.21-5.34 (m; 1 H); 4.82 (q; J=6.60 Hz; 0.5 H); 4.64 (d; J=8.78 Hz; 0.5 H); 4.55 (dd; J=14.11; 8.21 Hz; 1 H); 4.19 (dd; J=19.17; 7.51 Hz; 1 H); 4.01-4.13 (m; 6 H); 3.92 (d; J=8.93 Hz; 0.5 H); 3.69-3.78 (br m; 0.5 H); 3.48-3.54 (m; 2 H); 3.30 (d; J=13.35 Hz; 0.5 H); 3.00-3.07 (m; 3 H); 2.89 (br s; 1.5 H); 2.53 (s; 1.5 H); 2.28-2.32 (m; 5 H); 1.77-1.85 (m; 6 H); 1.58-1.66 (m; 3 H); 1.28-1.40 (m; 2.5 H); 1.02 (s; 1.5 H). MS (ESI) m/z: 972.5 [M+H]⁺.

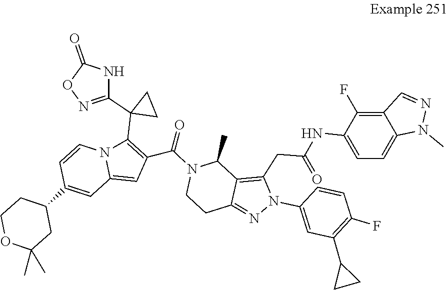 [SMILES: C[C@H]1c2c(nn(-c3ccc(F)c(C4CC4)c3)c2CC(=O)Nc2ccc3c(cnn3C)c2F)CCN1C(=O)c1cc2cc([C@H]3CCOC(C)(C)C3)ccn2c1C1(c2noc(=O)[nH]2)CC1]

### 2-((S)-2-(3-cyclopropyl-4-fluorophenyl)-5-(7-((S)-2,2-dimethyltetrahydro-2H-pyran-4-yl)-3-(1-(5-oxo-4,5-dihydro-1,2,4-oxadiazol-3-yl)cyclopropyl)indolizine-2-carbonyl)-4-methyl-4,5,6,7-tetrahydro-2H-pyrazolo[4,3-c]pyridin-3-yl)-N-(4-fluoro-1-methyl-1H-indazol-5-yl)acetamide

### Step 1: tert-butyl (S)-3-(2-(tert-butoxy)-2-oxoethyl)-2-(3-cyclopropyl-4-fluorophenyl)-4-methyl-2,4,6,7-tetrahydro-5H-pyrazolo[4,3-c]pyridine-5-carboxylate

Zinc (1.3 g, 0.25 mL, 20 mmol) was gently heated with a heat gun under an argon flow for 1 min. The heated solid was allowed to cool to room temperature. Anhydrous tetrahydrofuran (20 mL) and ethylene dibromide (0.19 g, 86 μL, 1.0 mmol) were added and the mixture was briefly heated with a heat gun to near reflux temperatures. The mixture was cooled to room temperature, after which chlorotrimethylsilane (0.11 g, 0.13 mL, 1.0 mmol) was added dropwise. The mixture was stirred at room temperature for 30 minutes. Tert-butyl bromoacetate (2.0 g, 1.5 mL, 1 eq, 10 mmol) was added dropwise and stirring was continued for 10 minutes. In a separate vial under argon atmosphere was added tert-butyl (S)-3-bromo-2-(3-cyclopropyl-4-fluorophenyl)-4-methyl-2,4,6,7-tetrahydro-5H-pyrazolo[4,3-c]pyridine-5-carboxylate (300 mg, 620 μmol) and anhydrous tetrahydrofuran (1.0 mL). The resulting solution was purged with argon for 5 minutes. Bis[tris(tert-butyl)phosphine]palladium (31.7 mg, 62.0 mol) was added and after 1 min, the previously prepared zincate mixture (2-(tert-butoxy)-2-oxoethyl)zinc(II) bromide (484 mg, 3.72 mL, 0.5 molar, 1.86 mmol) was added. The reaction mixture was placed in a preheated 70° C. heating block and stirred for 2 hours. After cooling to room temperature, water (0.1 mL) was added, and the suspension was filtered using a 0.45 μm nylon syringe filter. After concentration in vacuo, the residue was purified by flash column chromatography eluting with a gradient 5→50% ethyl acetate in heptane. MS (ESI) m/z: 486.3 [M+H]⁺.

### Step 2: tert-butyl (S)-2-(3-cyclopropyl-4-fluorophenyl)-3-(2-((4-fluoro-1-methyl-1H-indazol-5-yl)amino)-2-oxoethyl)-4-methyl-2,4,6,7-tetrahydro-5H-pyrazolo[4,3-c]pyridine-5-carboxylate

At room temperature, tert-butyl (S)-3-(2-(tert-butoxy)-2-oxoethyl)-2-(3-cyclopropyl-4-fluorophenyl)-4-methyl-2,4,6,7-tetrahydro-5H-pyrazolo[4,3-c]pyridine-5-carboxylate (194 mg, 400 mol) was dissolved in dichloromethane (1.5 mL) after which a 4.0 M HCl solution in 1,4-dioxane (146 mg, 999 μL, 4 molar, 4.00 mmol) was added. The mixture was stirred at room temperature for 2.5 hours. Additional 4.0 M HCl in 1,4-dioxane (72.8 mg, 499 μL, 4 molar, 2.00 mmol) was added, and the resulting mixture was stirred at room temperature for 1.5 hours. Additional 4.0 M HCl in 1,4-dioxane (72.8 mg, 499 μL, 4 molar, 2.00 mmol) was added, and the resulting mixture was stirred at room temperature for 2 hours. Additional 4.0 M HCl in 1,4-dioxane (146 mg, 999 μL, 4 molar, 4.00 mmol) was added, and the resulting mixture was stirred at room temperature for 16.5 hours. After concentration in vacuo, the residue obtained was dissolved in dichloromethane (3 mL). Di-tert-butyl dicarbonate (95.9 mg, 101 μL, 1.1 eq, 439 mol) and triethylamine (809 mg, 1.11 mL, 20 eq, 7.99 mmol) were added subsequently and, after stirring at room temperature for 2 hours the reaction mixture was concentrated in vacuo. The residue obtained was dissolved in anhydrous N-methyl-2-pyrrolidone (3.0 mL). 1-[bis(dimethylamino)methylene]-1H-1,2,3-triazolo[4,5-b]pyridinium 3-oxid hexafluorophosphate (183 mg, 480 mol) and N,N-diisopropylethylamine (207 mg, 279 μL, 1.60 mmol) were added subsequently and the resulting mixture was stirred at room temperature for 8 min. 4-fluoro-1-methyl-1H-indazol-5-amine (72.7 mg, 440 mol) was added, and the mixture was stirred at room temperature for 16 hours, followed by addition of 1-[bis(dimethylamino)methylene]-1H-1,2,3-triazolo[4,5-b]pyridinium 3-oxid hexafluorophosphate (183 mg, 480 mol) and N,N-diisopropylethylamine (103 mg, 139 μL, 800 mol). The resulting mixture was stirred at room temperature for 10 min, followed by addition of 4-fluoro-1-methyl-1H-indazol-5-amine (72.7 mg, 440 μmol). The resulting mixture was stirred at room temperature for 2.5 hours. The reaction mixture was filtered over a 0.45 μm nylon syringe filter, diluted with dimethyl sulfoxide (2.0 mL) and purified by preparative HPLC (Reprosil-pur Acidic C18-HD, 150*25 mm*10 m) using a gradient of 30→70% acetonitrile (0.1% v/v formic acid) in water (0.1% v/v formic acid). MS (ESI) m/z: 577.2 [M+H]⁺.

### Step 3: 2-((S)-5-(3-(1-cyanocyclopropyl)-7-((S)-2,2-dimethyltetrahydro-2H-pyran-4-yl)indolizine-2-carbonyl)-2-(3-cyclopropyl-4-fluorophenyl)-4-methyl-4,5,6,7-tetrahydro-2H-pyrazolo[4,3-c]pyridin-3-yl)-N-(4-fluoro-1-methyl-1H-indazol-5-yl)acetamide

A 4.0 M HCl solution in 1,4-dioxane (134 mg, 917 μL, 4 molar, 3.67 mmol) was added to a solution of tert-butyl (S)-2-(3-cyclopropyl-4-fluorophenyl)-3-(2-((4-fluoro-1-methyl-1H-indazol-5-yl)amino)-2-oxoethyl)-4-methyl-2,4,6,7-tetrahydro-5H-pyrazolo[4,3-c]pyridine-5-carboxylate (141 mg, 245 mol) in dichloromethane (2 mL). The resulting mixture was stirred at room temperature for 2.5 hours. The reaction mixture was concentrated in vacuo. In a separate vial, (S)-3-(1-cyanocyclopropyl)-7-(2,2-dimethyltetrahydro-2H-pyran-4-yl)indolizine-2-carboxylic acid (99.5 mg, 294 μmol) was dissolved in anhydrous N-methyl-2-pyrrolidone (2.0 mL) and 1-[bis(dimethylamino)methylene]-1H-1,2,3-triazolo[4,5-b]pyridinium 3-oxid hexafluorophosphate (121 mg, 319 mol) and N,N-diisopropylethylamine (158 mg, 213 μL, 1.23 mmol). The resulting mixture was stirred at room temperature for 15 minutes. This mixture was added to a solution of the previously obtained amine and N,N-diisopropylethylamine (158 mg, 213 μL, 1.23 mmol) in anhydrous N-methyl-2-pyrrolidone (0.5 mL). The resulting mixture was stirred at room temperature for 2 hours, after which it was filtered over 0.45 μm nylon syringe filter, diluted with dimethyl sulfoxide (2 mL) and purified by preparative HPLC (Reprosil-pur Acidic C18-HD, 150*25 mm*10 m) using a gradient of 30→70% acetonitrile (0.1% v/v formic acid) in water (0.1% v/v formic acid). MS (ESI) m/z: 797.2 [M+H]⁺.

### Step 4: 2-((S)-2-(3-cyclopropyl-4-fluorophenyl)-5-(7-((S)-2,2-dimethyltetrahydro-2H-pyran-4-yl)-3-(1-(5-oxo-4,5-dihydro-1,2,4-oxadiazol-3-yl)cyclopropyl)indolizine-2-carbonyl)-4-methyl-4,5,6,7-tetrahydro-2H-pyrazolo[4,3-c]pyridin-3-yl)-N-(4-fluoro-1-methyl-1H-indazol-5-yl)acetamide

A 50% aqueous hydroxylamine solution (82.9 mg, 76.9 μL, 50% wt, 1.25 mmol) was added to a solution of 2-((S)-5-(3-(1-cyanocyclopropyl)-7-((S)-2,2-dimethyltetrahydro-2H-pyran-4-yl)indolizine-2-carbonyl)-2-(3-cyclopropyl-4-fluorophenyl)-4-methyl-4,5,6,7-tetrahydro-2H-pyrazolo[4,3-c]pyridin-3-yl)-N-(4-fluoro-1-methyl-1H-indazol-5-yl)acetamide (100 mg, 125 μmol) in dimethyl sulfoxide (3.0 mL). The resulting mixture was stirred at room temperature for 17 hours. The reaction mixture was poured into water (30 mL), and the resulting mixture was extracted with ethyl acetate (3×30 mL). The combined organic extracts were dried over anhydrous Na₂SO₄ and concentrated in vacuo. The residue obtained was dissolved in in dimethyl sulfoxide (1.25 mL) after which 1,8-diazabicyclo(5.4.0)undec-7-ene (71.4 mg, 70.7 μL, 469 mol) and 1,1′-carbonyldiimidazole (60.8 mg, 375 mol) were added subsequently. The reaction mixture was stirred at room temperature for 4 hours, filtered over a 0.45 μm nylon syringe filter and purified by preparative HPLC (Reprosil-pur Basic C18-HD, 150*25 mm*10 m) using a gradient of 15→55% acetonitrile in water (10 mM NH₄HCO₃ pH 9.5). ¹H NMR (400 MHz, CDCl₃, rotamers) δ 11.97-11.31 (m, 1H), 8.14 (d, J=7.3 Hz, 1H), 8.07-7.89 (m, 2H), 7.82-7.37 (m, 1H), 7.31-7.16 (m, 2.5H), 7.14-6.94 (m, 2.5H), 6.65-6.45 (m, 1H), 6.42-6.31 (m, 1H), 6.23-5.66 (m, 1H), 5.41-4.83 (m, 0.5H), 4.69-4.18 (m, 1H), 4.11-4.01 (m, 3H), 3.92-3.71 (m, 3H), 3.64-3.46 (m, 1H), 3.45-3.19 (m, 1H), 3.18-2.76 (m, 3H), 2.68-2.40 (m, 0.5H), 2.19-2.01 (m, 1.5H), 1.96-1.87 (m, 0.5H), 1.81-1.37 (m, 8H), 1.37-1.22 (m, 6.5H), 1.21-1.10 (m, 1.5H), 1.02-0.85 (m, 2H), 0.76-0.63 (m, 2H); MS (ESI) m/z: 856.2 [M+H]⁺.

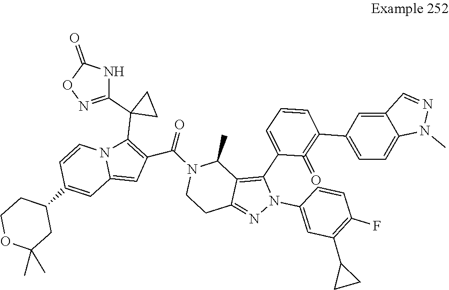

### 3-(1-(2-((4S)-2-(3-cyclopropyl-4-fluorophenyl)-4-methyl-3-(1-(1-methyl-1H-indazol-5-yl)-2-oxo-1,2-dihydropyridin-3-yl)-4,5,6,7-tetrahydro-2H-pyrazolo[4,3-c]pyridine-5-carbonyl)-7-((S)-2,2-dimethyltetrahydro-2H-pyran-4-yl)indolizin-3-yl)cyclopropyl)-1,2,4-oxadiazol-5(4H)-one

### Step 1: tert-butyl (4S)-2-(3-cyclopropyl-4-fluorophenyl)-3-(2-methoxypyridin-3-yl)-4-methyl-2,4,6,7-tetrahydro-5H-pyrazolo[4,3-c]pyridine-5-carboxylate

A solution of potassium triphosphate (556 mg, 2.62 mmol) in H₂O (2.5 mL) was added to a solution of (2-methoxypyridin-3-yl)boronic acid (267 mg, 1.75 mmol) and tert-butyl (S)-3-bromo-2-(3-cyclopropyl-4-fluorophenyl)-4-methyl-2,4,6,7-tetrahydro-5H-pyrazolo[4,3-c]pyridine-5-carboxylate (393 mg, 873 μmol) in tert-amyl alcohol (8.5 mL). The reaction mixture was sparged with argon for 10 min, before 1,1′-bis(di-tert-butylphosphino) ferrocene (2′-amino-1,1′-biphenyl-2-yl) palladium (II) methanesulfonate (73.0 mg, 87.3 μmol) was added. The reaction mixture was sparged with argon for an additional 5 min, heated 60° C., and stirred for 12 hours. After cooling to rt, the reaction mixture was filtered through a Celite® pad and washed with DCM (50 mL×3). The filtrate was concentrated, and the material was purified by flash chromatography (25 g silica gel cartridge, ISCO Combiflash, UV detection @254 nm) eluting with an increasing proportion of EtOAc (0 to 100%) in hexanes. MS (ESI) m/z: 479.5 [M+H]+.

### Step 2: tert-butyl (4S)-2-(3-cyclopropyl-4-fluorophenyl)-3-(2-methoxypyridin-3-yl)-4-methyl-2,4,6,7-tetrahydro-5H-pyrazolo[4,3-c]pyridine-5-carboxylate

A solution of tert-butyl-(4S)-2-(3-cyclopropyl-4-fluorophenyl)-3-(2-methoxypyridin-3-yl)-4-methyl-2,4,6,7-tetrahydro-5H-pyrazolo[4,3-c]pyridine-5-carboxylate (405 mg, 846 μmol) in acetic acid (5 mL) was treated with a solution of HBr (342 mg, 232 μL, 4.23 mmol, 33% wt in AcOH) followed with stirring at 60° C. for 30 min. The reaction mixture was then diluted with MeOH (15 mL), and the volatiles were removed under reduced pressure. The residue was dissolved in DCM (6 mL), then triethylamine (590 μL, 4.23 mmol) and Boc₂O (203 mg, 214 μL, 931 μmol) were then added. The reaction mixture was stirred at rt for 1 h. The volatiles were removed under reduced pressure, and the residue was purified by flash chromatography (25 g silica gel cartridge, ISCO Combiflash, UV detection @254 nm) eluting with an increasing proportion of MeOH (0 to 30%) in DCM. MS (ESI) m/z: 465.2 [M+H]⁺.

### Step 3. tert-butyl (4S)-2-(3-cyclopropyl-4-fluorophenyl)-4-methyl-3-(1-(1-methyl-1H-indazol-5-yl)-2-oxo-1,2-dihydropyridin-3-yl)-2,4,6,7-tetrahydro-5H-pyrazolo[4,3-c]pyridine-5-carboxylate

Potassium phosphate (252 mg, 1.19 mmol), copper(I) iodide (151 mg, 792 μmol), and methyl[2-(methylamino)ethyl]amine (34.9 mg, 42.5 μL, 396 μmol) were sequentially added to a solution of tert-butyl (4S)-2-(3-cyclopropyl-4-fluorophenyl)-4-methyl-3-(2-oxo-1,2-dihydropyridin-3-yl)-2,4,6,7-tetrahydro-5H-pyrazolo[4,3-c]pyridine-5-carboxylate 7 (184 mg, 396 μmol) and 5-bromo-1-methyl-1H-indazole (167 mg, 792 μmol) in 1,4-dioxane (2.5 mL) at rt. The resulting mixture was heated at 110° C. and stirred for 1 hour. After cooling to rt, the volatiles were removed under reduced pressure and the residue was purified by flash chromatography (25 g silica gel cartridge, ISCO Combiflash, UV detection @254 nm) eluting with an increasing proportion of EtOAc (0 to 100%) in hexanes. MS (ESI) m/z: 595.5 [M+H]⁺.

### Step 4: 3-((4S)-2-(3-cyclopropyl-4-fluorophenyl)-4-methyl-4,5,6,7-tetrahydro-2H-pyrazolo[4,3-c]pyridin-3-yl)-1-(1-methyl-1H-indazol-5-yl)pyridin-2(1H)-one

A solution of tert-butyl (4S)-2-(3-cyclopropyl-4-fluorophenyl)-4-methyl-3-(1-(1-methyl-1H-indazol-5-yl)-2-oxo-1,2-dihydropyridin-3-yl)-2,4,6,7-tetrahydro-5H-pyrazolo[4,3-c]pyridine-5-carboxylate (84 mg, 0.14 mmol) in DCM (2 mL) was treated with HCl (0.57 mL, 1.4 mmol, 2.5 M in Et₂O) at rt, followed with stirring for 12 h. The reaction mixture was quenched with a saturated aqueous solution of NaHCO₃ (5 mL), extracted with 25% solution of iPrOH in CHCl₃ (20 mL×2). The combined organic layers were dried over Na₂SO₄, filtered and concentrated. The residue was purified by flash chromatography (12 g silica gel cartridge, ISCO Combiflash, UV detection @254 nm) eluting with an increasing proportion of MeOH (0 to 30%) in DCM. MS (ESI) m/z: 495.3 [M+H]+.

### Step 5. 1-(2-((4S)-2-(3-cyclopropyl-4-fluorophenyl)-4-methyl-3-(1-(1-methyl-1H-indazol-5-yl)-2-oxo-1,2-dihydropyridin-3-yl)-4,5,6,7-tetrahydro-2H-pyrazolo[4,3-c]pyridine-5-carbonyl)-7-((R) 2,2-dimethyltetrahydro-2H-pyran-4-yl)indolizin-3-yl)cyclopropane-1-carbonitrile

DIPEA (81 μL, 0.47 mmol) was added to a solution of (R)-3-(1-cyanocyclopropyl)-7-(2,2-dimethyltetrahydro-2H-pyran-4-yl)indolizine-2-carboxylic acid (47 mg, 0.14 mmol), 3-((4S)-2-(3-cyclopropyl-4-fluorophenyl)-4-methyl-4,5,6,7-tetrahydro-2H-pyrazolo[4,3-c]pyridin-3-yl)-1-(1-methyl-1H-indazol-5-yl)pyridin-2(1H)-one (46 mg, 93 μmol) and HATU (53 mg, 0.14 mmol) in DMF (1.4 mL). The mixture was then heated at 60° C. and stirred for 4 hours. After cooling to rt, the reaction mixture was quenched by the addition of a saturated aqueous solution of NH₄Cl (5 mL), extracted with 25% solution of iPrOH in CHCl₃ (10 mL×3). The combined organic layers were washed with water (10 mL), brine (10 mL), dried over Na₂SO₄, filtered and concentrated. The residue was purified by flash chromatography (80 g silica gel cartridge, ISCO Combiflash, UV detection @254 nm) eluting with an increasing proportion of MeOH (0 to 30%) in DCM. m/z: 815.5 [M+H]⁺.

### Step 6. (Z)-1-(2-((4S)-2-(3-cyclopropyl-4-fluorophenyl)-4-methyl-3-(1-(1-methyl-1H-indazol-5-yl)-2-oxo-1,2-dihydropyridin-3-yl)-4,5,6,7-tetrahydro-2H-pyrazolo[4,3-c]pyridine-5-carbonyl)-7-((R)-2,2-dimethyltetrahydro-2H-pyran-4-yl)indolizin-3-yl)-N′-hydroxycyclopropane-1-carboximidamide

A solution of 1-(2-((4S)-2-(3-cyclopropyl-4-fluorophenyl)-4-methyl-3-(1-(1-methyl-1H-indazol-5-yl)-2-oxo-1,2-dihydropyridin-3-yl)-4,5,6,7-tetrahydro-2H-pyrazolo[4,3-c]pyridine-5-carbonyl)-7-((R)-2,2-dimethyltetrahydro-2H-pyran-4-yl)indolizin-3-yl)cyclopropane-1-carbonitrile (46 mg, 0.056 mmol) in ethanol (1 mL) was treated with hydroxylamine hydrochloride (22 mg, 0.31 mmol) and sodium bicarbonate (24 mg, 0.28 mmol) at rt. The resulting mixture was heated to 75° C. and stirred for 2 h. After cooling to rt, the reaction mixture was filtered and washed with ethanol (5 mL). The volatiles were removed under reduced pressure. The residue was used in the following step without further purification. m/z: 848.5 [M+H]⁺.

### Step 7. 3-(1-(2-((4S)-2-(3-cyclopropyl-4-fluorophenyl)-4-methyl-3-(1-(1-methyl-1H-indazol-5-yl)-2-oxo-1,2-dihydropyridin-3-yl)-4,5,6,7-tetrahydro-2H-pyrazolo[4,3-c]pyridine-5-carbonyl)-7-((R)-2,2-dimethyltetrahydro-2H-pyran-4-yl)indolizin-3-yl)cyclopropyl)-1,2,4-oxadiazol-5(4H)-one

N, N′-carbonyl diimidazole (23 mg, 0.14 mmol) was added to a solution of (Z)-1-(2-((4S)-2-(3-cyclopropyl-4-fluorophenyl)-4-methyl-3-(1-(1-methyl-1H-indazol-5-yl)-2-oxo-1,2-dihydropyridin-3-yl)-4,5,6,7-tetrahydro-2H-pyrazolo[4,3-c]pyridine-5-carbonyl)-7-((R)-2,2-dimethyltetrahydro-2H-pyran-4-yl)indolizin-3-yl)-N′-hydroxycyclopropane-1-carboximidamide (48 mg, 0.057 mmol) and 1,8-diazabicyclo [5.4.0]undec-7-ene (22 mg, 21 μL, 0.14 mmol) in DMSO (1.5 mL) at rt, followed with stirring for 12 h. After 12 h, additional N, N′-carbonyl diimidazole (23 mg, 0.14 mmol) and 1,8-diazabicyclo [5.4.0]undec-7-ene (22 mg, 21 μL, 0.14 mmol) were added and the reaction mixture was stirred for another 6 h at rt. The reaction mixture was diluted with H₂O (10 mL), washed with a 25% solution of iPrOH in CHCl₃ (25 mL×2). The combined organic layers were washed with brine (20 mL), dried over Na₂SO₄, filtered and concentrated under reduced pressure. The residue was purified on ACCQ prep (Column: Gemini 5 μm, NX—C18, 110 A, 150×21.1 mm; Flow rate 20 ml/min, eluent: increasing proportion (0-100%) of CH₃CN in 1% Amb). ¹H NMR (CH₃CN-d₃, 400 MHz): δ_{H} 7.98-8.23 (2H, m), 8.18-8.02 (2H, m), 7.73-7.54 (3H, m), 7.33-7.06 (4H, m), 6.93-6.84 (1H, m), 6.71 (1H, d, J=7.0 Hz), 6.50 (1H, s), 6.43-6.38 (1H, m), 6.21-5.88 (1H, m), 5.01-4.26 (1H, m), 4.10 (3H, d, J=3.9 Hz), 3.77 (2H, d, J=7.8 Hz), 3.55-3.34 (2H, m), 3.00-2.79 (2H, m), 2.52-2.05 (2H, m), 1.82-1.47 (5H, m), 1.40-1.20 (10H, m), 1.02-0.97 (2H, m), 0.58 (2H, dt, J=49.5, 4.9 Hz). ¹⁹F NMR (CH₃CN-d₃, 376 MHz): δ_{F} −123.7 (1F, m). m/z: 874.3 [M+H]⁺.

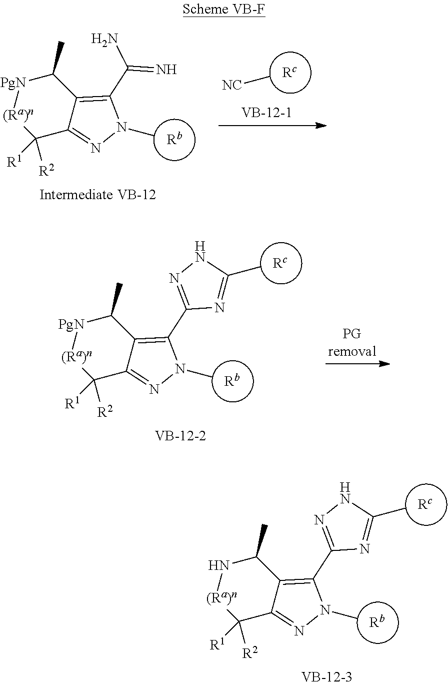

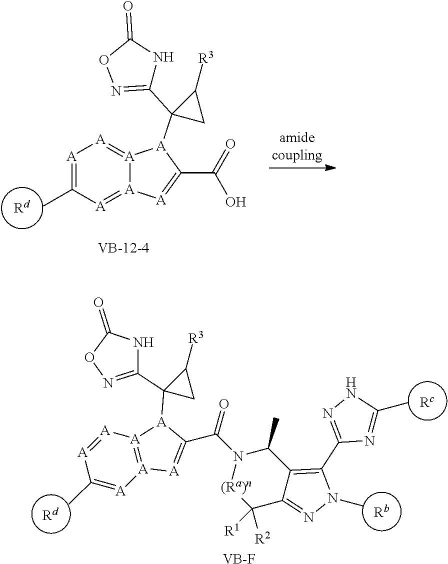

Compounds of formula VB—F are prepared from intermediate VB-12 by Cu-promoted coupling with Cyano-substituted reagents VB-12-1 providing triazoles VB-12-2. Removal of the protecting group (e.g., acidic conditions) of VB-12-2 provides amine VB-12-3 that is then coupled with carboxylic acid VB-12-4 to provide compounds of formula VB—F.

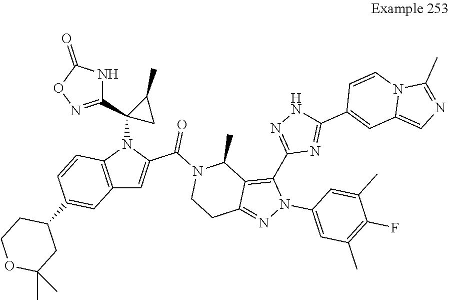 [SMILES: Cc1cc(-n2nc3c(c2-c2n[nH]c(-c4ccn5c(C)ncc5c4)n2)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F]

### 3-((1S,2S)-1-(5-((S)-2,2-dimethyltetrahydro-2H-pyran-4-yl)-2-((S)-2-(4-fluoro-3,5-dimethylphenyl)-4-methyl-3-(5-(3-methylimidazo[1,5-a]pyridin-7-yl)-1H-1,2,4-triazol-3-yl)-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one

### Step 1: tert-butyl (S)-2-(4-fluoro-3,5-dimethylphenyl)-4-methyl-3-(3-(3-methylimidazo[1,5-a]pyridin-7-yl)-1H-1,2,4-triazol-5-yl)-2,4,6,7-tetrahydro-5H-pyrazolo[4,3-c]pyridine-5-carboxylate

To a solution of tert-butyl (S)-3-carbamimidoyl-2-(4-fluoro-3,5-dimethylphenyl)-4-methyl-2,4,6,7-tetrahydro-5H-pyrazolo[4,3-c]pyridine-5-carboxylate (80 mg, 0.20 mmol), 3-methylimidazo[1,5-a]pyridine-7-carbonitrile (31 mg, 0.20 mmol) and Na₂CO₃ (84 mg, 0.80 mmol) in PhCH₃ (1 mL) was added Cu(OAc)₂ (36 mg, 8.2 μL, 0.20 mmol), and the resulting mixture was stirred at 110° C. under O₂ for 16 hour. The mixture was diluted with H₂O (8 mL) extracted with EtOAc (5 mL, 3×). The combined organic extracts were dried over anhydrous Na₂SO₄, filtered, and concentrated under reduced pressure. Then the crude product was purified by pre-HPLC (YMC-Actus Triart C18 150*30 mm*5 m Condition water(0.1% TFA)-ACN Begin B 28 End B 58 Gradient Time(min) 11 100% B Hold Time 1.1 Flow Rate(40 mL/min) Injections 2). m/z: 557.3 [M+H]⁺.

### Step 2: (S)-2-(4-fluoro-3,5-dimethylphenyl)-4-methyl-3-(3-(3-methylimidazo[1,5-a]pyridin-7-yl)-1H-1,2,4-triazol-5-yl)-4,5,6,7-tetrahydro-2H-pyrazolo[4,3-c]pyridine

To a solution of tert-butyl (S)-2-(4-fluoro-3,5-dimethylphenyl)-4-methyl-3-(3-(3-methylimidazo[1,5-a]pyridin-7-yl)-1H-1,2,4-triazol-5-yl)-2,4,6,7-tetrahydro-5H-pyrazolo[4,3-c]pyridine-5-carboxylate (37.00 mg, 66.47 mol)in dioxane (1 mL) was added 2M HCl-dioxane (2.423 mg, 33.23 μL, 2 molar, 66.47 mol), and the resulting mixture was stirred at 25° C. for 2 hour. The reaction was concentrated and used for the next step without further purification. m/z: 457.2 [M+H]⁺.

### Step 3: 3-((1S,2S)-1-(5-((S)-2,2-dimethyltetrahydro-2H-pyran-4-yl)-2-((S)-2-(4-fluoro-3,5-dimethylphenyl)-4-methyl-3-(5-(3-methylimidazo[1,5-a]pyridin-7-yl)-1H-1,2,4-triazol-3-yl)-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one

To a solution of (S)-2-(4-fluoro-3,5-dimethylphenyl)-4-methyl-3-(3-(3-methylimidazo[1,5-a]pyridin-7-yl)-1H-1,2,4-triazol-5-yl)-4,5,6,7-tetrahydro-2H-pyrazolo[4,3-c]pyridine (30.00 mg, 1 eq, 65.71 mol), 5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carboxylic acid (35.0 mg, 85.1 mol), DIPEA (42.47 mg, 57.2 μL, 328.6 mol) in DMF (1 mL) was added HATU (37.48 mg, 98.57 mol), and the resulting mixture was stirred at 25° C. for 16 hour. The crude product was purified by prep-HPLC (45% to 65% MeCN/H2O+0.1% TFA gradient). ¹H NMR (400 MHz, acetonitrile-d₃) δ 11.65-11.41 (m, 1H), 8.31 (s, 1H), 8.17-8.08 (m, 1H), 8.00 (d, J=6.9 Hz, 1H), 7.82 (s, 1H), 7.79-7.47 (m, 3H), 7.44 (d, J=8.6 Hz, 1H), 7.33-7.26 (m, 1H), 7.21 (d, J=6.9 Hz, 1H), 7.12 (d, J=6.2 Hz, 1H), 7.06 (d, J=6.3 Hz, 1H), 6.93-6.80 (m, 1H), 6.09-5.97 (m, 1H), 5.68-5.58 (m, 1H), 4.93-4.78 (m, 1H), 4.41 (br dd, J=5.4, 13.5 Hz, 1H), 3.85-3.64 (m, 3H), 3.16-2.95 (m, 3H), 2.88-2.78 (m, 4H), 1.87 (br d, J=6.7 Hz, 2H), 1.83-1.72 (m, 4H), 1.71-1.65 (m, 2H), 1.63-1.53 (m, 5H), 1.44-1.34 (m, 1H), 1.31-1.25 (m, 4H), 1.20 (s, 2H), 1.17 (d, J=6.0 Hz, 2H), 1.12 (s, 1H), 1.01 (d, J=6.1 Hz, 1H). m/z: 850.4 [M+H]⁺.

The following examples in table 22 were prepared according to scheme VB—F using the procedures outlined in the synthesis of example 253.

TABLE 22

| | | | MS |
|---|---|---|---|
| Ex. | Structure | Name | (M + 1) |
| 254 | 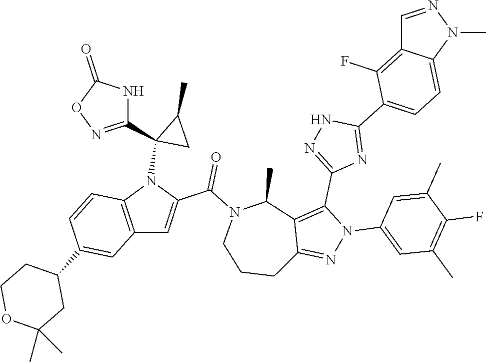 [SMILES: Cc1cc(-n2nc3c(c2-c2n[nH]c(-c4ccc5c(cnn5C)c4F)n2)[C@H](C)N(C(=O)c2cc4cc([C@H]5CCOC(C)(C)C5)ccc4n2[C@@]2(c4noc(=O)[nH]4)C[C@@H]2C)CCC3)cc(C)c1F] | 3-((1S,2S)-1-(5-((S)-2,2- dimethyltetrahydro-2H-pyran-4- yl)-2-((S)-3-(5-(4-fluoro-1- methyl-1H-indazol-5-yl)-1H- 1,2,4-triazol-3-yl)-2-(4-fluoro- 3,5-dimethylphenyl)-4-methyl- 2,4,5,6,7,8- hexahydropyrazolo[4,3- c]azepine-5-carbonyl)-1H-indol- 1-yl)-2-methylcyclopropyl)- 1,2,4-oxadiazol-5(4H)-one | 882.4 |
| 255 | 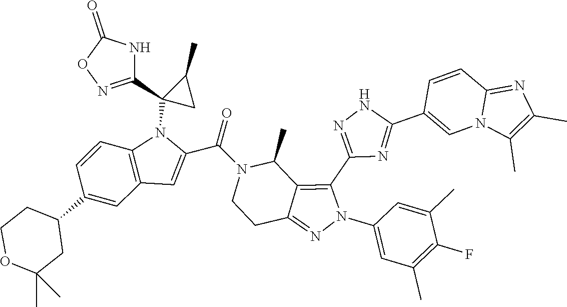 | 3-((1S,2S)-1-(2-((S)-3-(5-(2,3- dimethylimidazo[1,2-a]pyridin- 6-yl)-1H-1,2,4-triazol-3-yl)-2-(4- fluoro-3,5-dimethylphenyl)-4- methyl-4,5,6,7-tetrahydro-2H- pyrazolo[4,3-c]pyridine-5- carbonyl)-5-((S)-2,2- dimethyltetrahydro-2H-pyran-4- yl)-1H-indol-1-yl)-2- methylcyclopropyl)-1,2,4- oxadiazol-5(4H)-one | 864.4 |

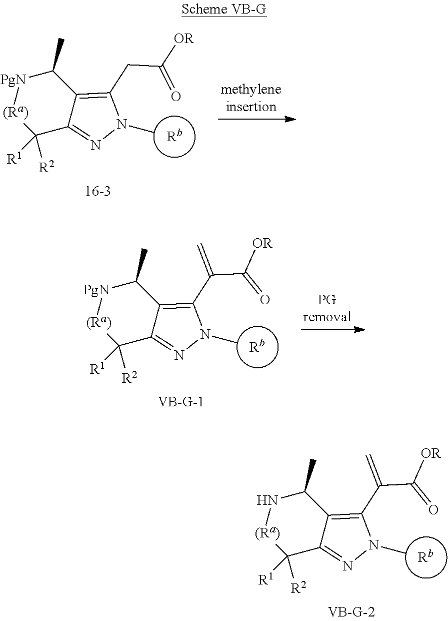

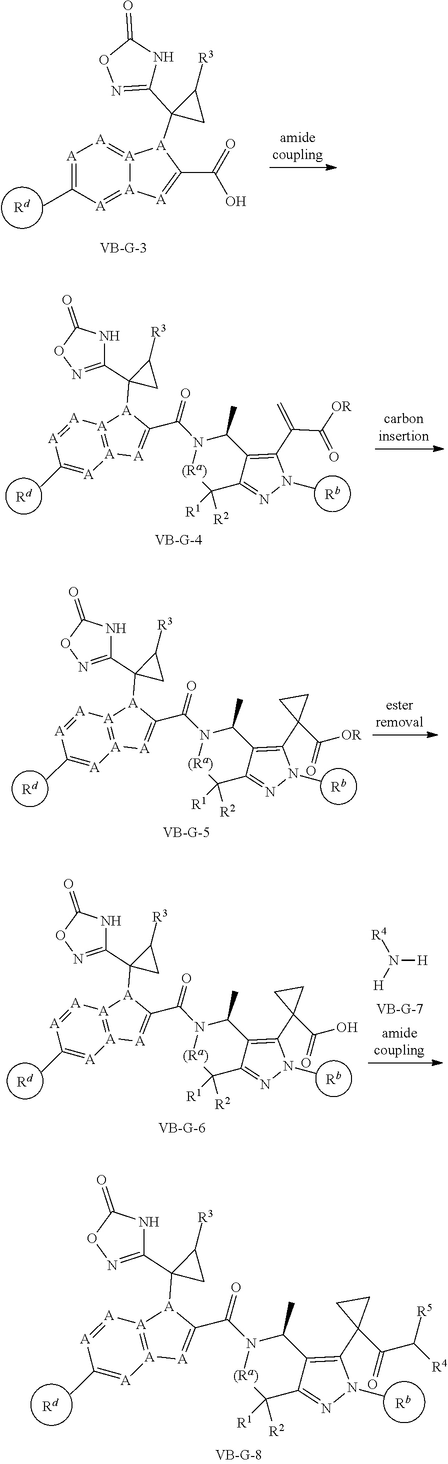

Compounds of formula VB-G-8 are prepared from methylene insertion using paraformaldehyde and base to first provide VB-G-1. Removal of the protecting group (e.g., acidic conditions) of VB-G-1 provides amine VB-G-2 that is then coupled with carboxylic acid VB-G-3 to provide VB-G-4. Carbon insertion using trimethyl(oxo)sulfonium iodide into VB-G-4 provides cyclopropane VB-G-5 which upon removal of the ester (e.g., basic aqueous conditions)furnishes carboxylic acid VB-G-6. Subsequently, VB-G-6 is coupled with amine VB-G-7 to provide compounds of the formula VB-G-8.

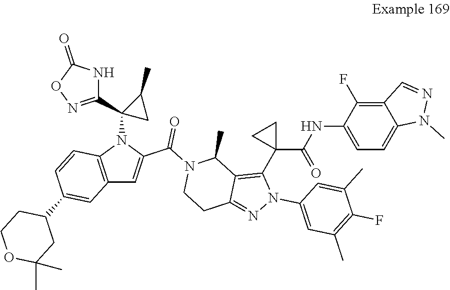

### 1-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)-N-(4-fluoro-1-methyl-1H-indazol-5-yl)cyclopropane-1-carboxamide

### Step 1: tert-butyl (S)-2-(4-fluoro-3,5-dimethylphenyl)-3-(3-methoxy-3-oxoprop-1-en-2-yl)-4-methyl-2,4,6,7-tetrahydro-5H-pyrazolo[4,3-c]pyridine-5-carboxylate

To a flask containing tert-butyl (S)-2-(4-fluoro-3,5-dimethylphenyl)-3-(2-methoxy-2-oxoethyl)-4-methyl-2,4,6,7-tetrahydro-5H-pyrazolo[4,3-c]pyridine-5-carboxylate (1.46 g, 3.38 mmol) was added paraformaldehyde (203 mg, 6.77 mmol) and potassium carbonate (468 mg, 3.38 mmol). The solids were suspended in DMF (33.8 mL) and then heated to 100° C. for 1 hour. The mixture was diluted with 10% LiCl aq. solution and extracted twice with EtOAc. The combined organic layers were washed with brine and then dried over Na₂SO₄, filtered, and concentrated under reduced pressure. The crude residue was purified by silica gel chromatography (0-80% hexanes/EtOAc) to provide the title compound. MS (ESI) m/z 444.4 [M+1]+.

### Step 2: methyl 2-(2-(4-fluoro-3,5-dimethylphenyl)-4,5,6,7-tetrahydro-2H-pyrazolo[4,3-c]pyridin-3-yl)acrylate

This step was performed in a similar fashion as in Example 1, Step 4 to provide the title compound. MS (ESI) m/z 344.1 [M+1]+.

### Step 3: methyl 2-(5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4,5,6,7-tetrahydro-2H-pyrazolo[4,3-c]pyridin-3-yl)acrylate

This step was performed in a similar fashion to Example 1, Step 5 employing 5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carboxylic acid as coupling partner to provide the title compound. MS (ESI) m/z 737.4 [M+1]+.

### Step 4: methyl 1-(5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4,5,6,7-tetrahydro-2H-pyrazolo[4,3-c]pyridin-3-yl)cyclopropane-1-carboxylate

To a stirred suspension of trimethyl(oxo)sulfonium iodide (39.4 mg, 179 mol) and methyl 2-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)acrylate (120 mg, 163 mol) in MeCN (2.04 mL) was added 1-methyl-2,3,4,6,7,8-hexahydro-1H-pyrimido[1,2-a]pyrimidine (52.5 mg, 49.2 μL, 95% Wt, 326 mol) at room temperature. The resultant reaction mixture was heated to 60° C. for 1 hour. The mixture was concentrated under reduced pressure and then purified by silica gel column chromatography (0-100% EtOAc:Hexanes) to afford the title compound. MS (ESI) m/z 751.5 [M+1]+.

### Step 5: 1-(5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4,5,6,7-tetrahydro-2H-pyrazolo[4,3-c]pyridin-3-yl)cyclopropane-1-carboxylic acid

To a solution of methyl 1-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)cyclopropane-1-carboxylate (61 mg, 81 mol) in THF (0.81 mL) and MeOH (0.41 mL) was added a solution of LiOH (19 mg, 0.81 mL, 1 M in water, 0.81 mmol). The resulting mixture was stirred at 60° C. for 1 hour. At this time NaOH (16 mg, 0.10 mL, 4 M in water, 0.41 mmol) was added followed by THF (0.81 mL) and MeOH (0.41 mL). The reaction mixture was stirred at 60° C. for 18 hours. The reaction mixture was concentrated under a stream of N₂(g), acidified with 1N HCl to a pH of 1 and then extracted with EtOAc, dried over Na₂SO₄, filtered, and concentrated under reduced pressure to provide the title compound which was not purified further. MS (ESI) m/z 737.5 [M+1]+.

### Step 6: 1-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)-N-(4-fluoro-1-methyl-1H-indazol-5-yl)cyclopropane-1-carboxamide

To a solution of 1-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)cyclopropane-1-carboxylic acid (20 mg, 27 μmol), 4-fluoro-1-methyl-1H-indazol-5-amine, HCl (16 mg, 81 μmol) and TCFH (9.1 mg, 33 μmol) in MeCN (0.600 mL) was added NMI (22 mg, 22 μL, 0.27 mmol). The reaction mixture was stirred at room temperature for 30 minutes. The mixture was concentrated under a stream of N₂(g) and then purified using silica gel column chromatography 0 EtOAc/Hexanes) to provide the title compound. MS (ESI) m/z 884.5 [M+1]+.

The following examples in Table 23 were prepared according to scheme VB-G using the procedures outlined in the synthesis of example 169 immediately preceding this paragraph. For some examples the order of steps may be modified.

TABLE 23

| | | | MS |
|---|---|---|---|
| | | | (M + |
| Ex. | Structure | Name | 1) |
| 260 | 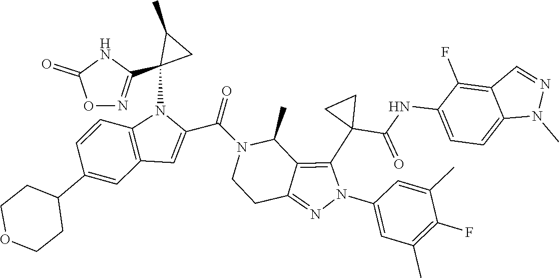 [SMILES: Cc1cc(-n2nc3c(c2C2(C(=O)Nc4ccc5c(cnn5C)c4F)CC2)[C@H](C)N(C(=O)c2cc4cc(C5CCOCC5)ccc4n2[C@@]2(c4noc(=O)[nH]4)C[C@@H]2C)CC3)cc(C)c1F] | N-(4-fluoro-1-methyl-1H-indazol-5- yl)-1-((S)-2-(4-fluoro-3,5- dimethylphenyl)-4-methyl-5-(1- ((1S,2S)-2-methyl-1-(5-oxo-4,5- dihydro-1,2,4-oxadiazol-3- yl)cyclopropyl)-5-(tetrahydro-2H- pyran-4-yl)-1H-indole-2-carbonyl)- 4,5,6,7-tetrahydro-2H-pyrazolo[4,3- c]pyridin-3-yl)cyclopropane-1- carboxamide | 856.4 |
| 262 | 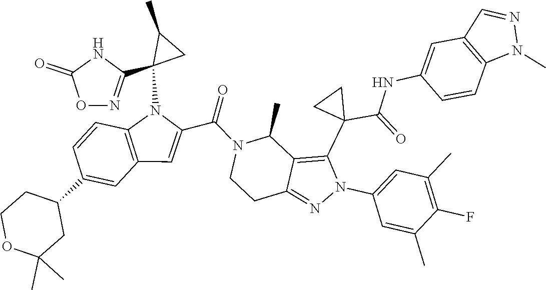 [SMILES: Cc1cc(-n2nc3c(c2C2(C(=O)Nc4ccc5c(cnn5C)c4)CC2)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | 1-((S)-5-(5-((S)-2,2- dimethyltetrahydro-2H-pyran-4-yl)- 1-((1S,2S)-2-methyl-1-(5-oxo-4,5- dihydro-1,2,4-oxadiazol-3- yl)cyclopropyl)-1H-indole-2- carbonyl)-2-(4-fluoro-3,5- dimethylphenyl)-4-methyl-4,5,6,7- tetrahydro-2H-pyrazolo[4,3- c]pyridin-3-yl)-N-(1-methyl-1H- indazol-5-yl)cyclopropane-1- carboxamide | 866.5 |
| 263 | 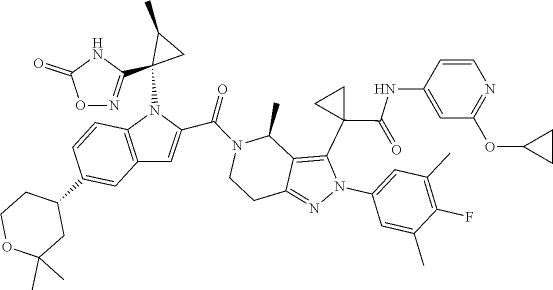 [SMILES: Cc1cc(-n2nc3c(c2C2(C(=O)Nc4ccnc(OC5CC5)c4)CC2)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | N-(2-cyclopropoxypyridin-4-yl)-1- ((S)-5-(5-((S)-2,2- dimethyltetrahydro-2H-pyran-4-yl)- 1-((1S,2S)-2-methyl-1-(5-oxo-4,5- dihydro-1,2,4-oxadiazol-3- yl)cyclopropyl)-1H-indole-2- carbonyl)-2-(4-fluoro-3,5- dimethylphenyl)-4-methyl-4,5,6,7- tetrahydro-2H-pyrazolo[4,3- c]pyridin-3-yl)cyclopropane-1- carboxamide | 869.5 |
| 264 | 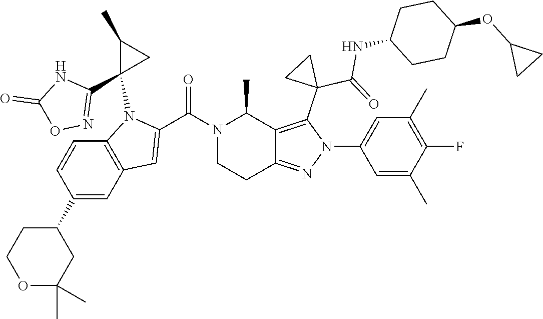 [SMILES: Cc1cc(-n2nc3c(c2C2(C(=O)N[C@H]4CC[C@H](OC5CC5)CC4)CC2)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | N-((1r,4S)-4- cyclopropoxycyclohexyl)-1-((S)-5- (5-((S)-2,2-dimethyltetrahydro-2H- pyran-4-yl)-1-((1S,2S)-2-methyl-1- (5-oxo-4,5-dihydro-1,2,4-oxadiazol- 3-yl)cyclopropyl)-1H-indole-2- carbonyl)-2-(4-fluoro-3,5- dimethylphenyl)-4-methyl-4,5,6,7- tetrahydro-2H-pyrazolo[4,3- c]pyridin-3-yl)cyclopropane-1- carboxamide | 875.0 |
| 265 | 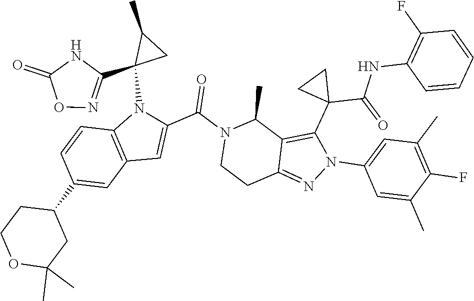 [SMILES: Cc1cc(-n2nc3c(c2C2(C(=O)Nc4ccccc4F)CC2)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | 1-((S)-5-(5-((S)-2,2- dimethyltetrahydro-2H-pyran-4-yl)- 1-((1S,2S)-2-methyl-1-(5-oxo-4,5- dihydro-1,2,4-oxadiazol-3- yl)cyclopropyl)-1H-indole-2- carbonyl)-2-(4-fluoro-3,5- dimethylphenyl)-4-methyl-4,5,6,7- tetrahydro-2H-pyrazolo[4,3- c]pyridin-3-yl)-N-(2- fluorophenyl)cyclopropane-1- carboxamide | 830.3 |
| 266 | 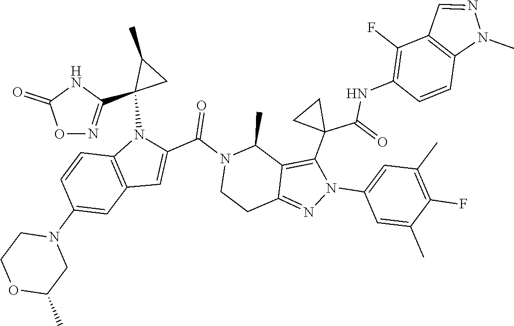 [SMILES: Cc1cc(-n2nc3c(c2C2(C(=O)Nc4ccc5c(cnn5C)c4F)CC2)[C@H](C)N(C(=O)c2cc4cc(N5CCO[C@@H](C)C5)ccc4n2[C@@]2(c4noc(=O)[nH]4)C[C@@H]2C)CC3)cc(C)c1F] | N-(4-fluoro-1-methyl-1H-indazol-5- yl)-1-((S)-2-(4-fluoro-3,5- dimethylphenyl)-4-methyl-5-(1- ((1S,2S)-2-methyl-1-(5-oxo-4,5- dihydro-1,2,4-oxadiazol-3- yl)cyclopropyl)-5-((S)-2- methylmorpholino)-1H-indole-2- carbonyl)-4,5,6,7-tetrahydro-2H- pyrazolo[4,3-c]pyridin-3- yl)cyclopropane-1-carboxamide | 871.4 |

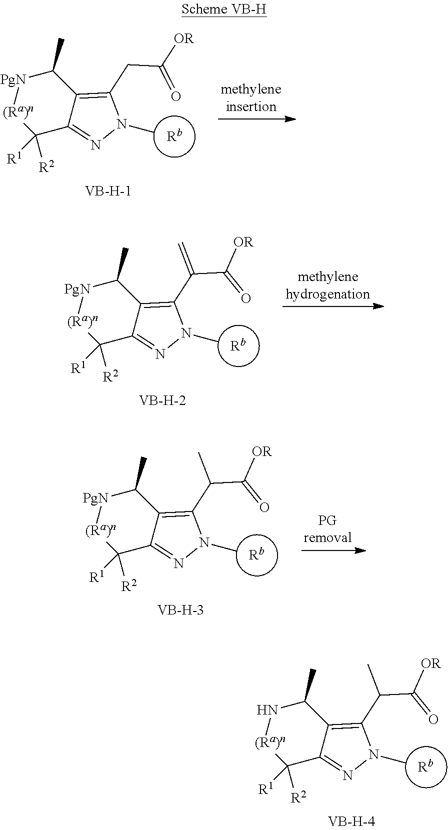

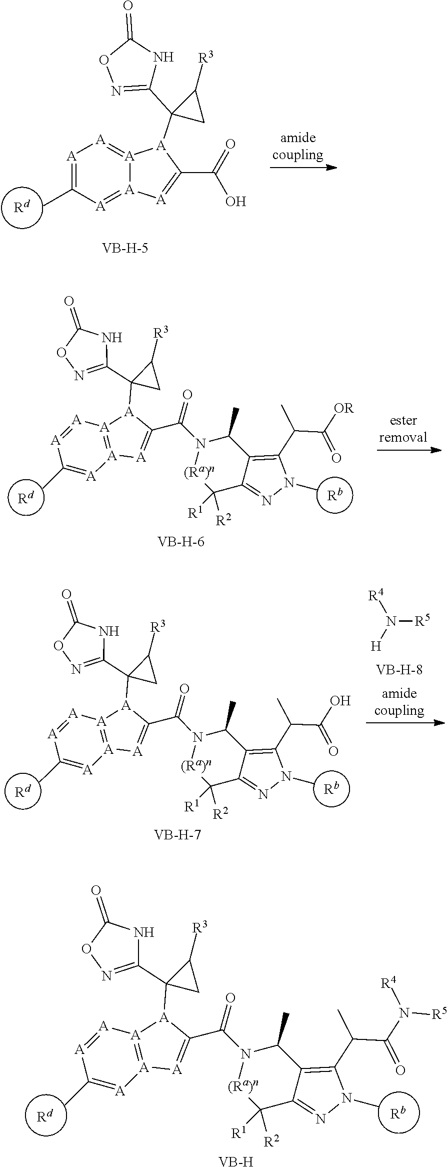

Compounds of formula VB—H are prepared from intermediate VB—H-1 through methylene insertion using paraformaldehyde and base to provide VB—H-2. Hydrogenation of VB—H-2 can proceed via metal mediated hydrogenation using metals such as Pd/C under hydrogen atmosphere to provide compound VB—H-3. Removal of the protecting group (e.g., acidic conditions) of VB—H-3 provides amine VB—H-4 that is then coupled with carboxylic acid VB—H-5 to provide VB—H-6. Removal of the ester (e.g., basic aqueous conditions) of VB—H-6 furnishes carboxylic acid VB—H-7. Subsequently, VB—H-7 is coupled with amine VB—H-8 to provide compounds of formula VB—H. Alternatively, the order of steps for some examples may be varied to facilitate the syntheses of the title compounds of formula VB—H.

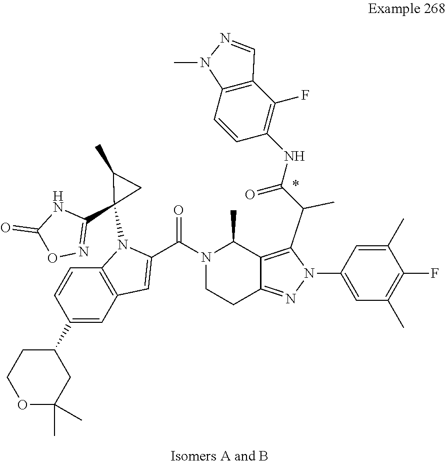 [SMILES: Cc1cc(-n2nc3c(c2C(C)C(=O)Nc2ccc4c(cnn4C)c2F)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F]

### (S)-2-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)-N-(4-fluoro-1-methyl-1H-indazol-5-yl)propenamide or (R)-2-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)-N-(4-fluoro-1-methyl-1H-indazol-5-yl)propenamide

### Step 1: methyl 2-((4S)-5-(5-(2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)acrylate

A vial was charged with methyl 2-((4S)-5-(5-(2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)acetate (100 mg, 138 mol), potassium carbonate (19.1 mg, 138 mol), and paraformaldehyde (8.29 mg, 276 mol) followed by addition of DMF (1.4 mL). The mixture was heated to 100° C. for 1 hour. It was then cooled to room temperature, concentrated, and purified by silica gel chromatography (0 to 100% EtOAc/Hexanes) to provide the title compound. MS (ESI) m/z 737.6 [M+H]+.

### Step 2: tert-butyl (4S)-2-(4-fluoro-3,5-dimethylphenyl)-3-(1-methoxy-1-oxopropan-2-yl)-4-methyl-2,4,6,7-tetrahydro-5H-pyrazolo[4,3-c]pyridine-5-carboxylatetert-butyl (4S)-2-(4-fluoro-3,5-dimethylphenyl)-3-(1-methoxy-1-oxopropan-2-yl)-4-methyl-2,4,6,7-tetrahydro-5H-pyrazolo[4,3-c]pyridine-5-carboxylate

A vial was charged with tert-butyl (S)-2-(4-fluoro-3,5-dimethylphenyl)-3-(3-methoxy-3-oxoprop-1-en-2-yl)-4-methyl-2,4,6,7-tetrahydro-5H-pyrazolo[4,3-c]pyridine-5-carboxylate (30 mg, 68 mol) and platinic oxide (3.1 mg, 0.30 μL, 14 μmol). Ethyl acetate (0.68 mL) was added then subjected to an atmosphere of H₂(g). Stirred at room temperature for 30 minutes. The reaction mixture was filtered through Celite®, rinsed with excess EtOAc and MeOH, then concentrated to afford the title compound. MS (ESI) m/z 446.4 [M+H]+.

Steps 3-6 were performed in a similar fashion as in Example 78 Steps 2-5. The final compounds were purified then separated by SFC (40% MeOH with 0.1% NH4OH) to provide the title compound. Peak 1: ¹H NMR (500 MHz, DMSO-d₆) δ 11.86 (d, J=102.1 Hz, 1H), 9.84-9.57 (m, 1H), 8.12 (s, 1H), 7.59-7.31 (m, 4H), 7.30-7.22 (m, 2H), 6.91 (d, J=5.5 Hz, 1H), 6.45-5.46 (m, 2H), 4.62-4.28 (m, 1H), 4.04 (d, J=10.0 Hz, 3H), 3.94-3.88 (m, 1H), 3.71 (d, J=6.0 Hz, 3H), 3.53-3.45 (m, 1H), 3.18-2.99 (m, 2H), 2.86 (d, J=15.3 Hz, 1H), 2.32-2.27 (m, 5H), 1.81-1.41 (m, 1OH), 1.32-1.23 (m, 6H), 1.20-1.13 (m, 4H), 0.98-0.80 (m, 1H). Peak 2: ¹H NMR (500 MHz, DMSO-d₆) δ 11.82 (s, 1H), 9.74-9.57 (m, 1H), 8.23-8.08 (m, 1H), 7.56-7.32 (m, 4H), 7.28-7.20 (m, 1H), 7.19-7.09 (m, 2H), 6.94-6.84 (m, 1H), 5.88-5.76 (m, 1H), 4.35-4.22 (m, 1H), 4.06 (s, 3H), 3.97 (q, J=7.2 Hz, 1H), 3.75-3.62 (m, 3H), 3.16-2.97 (m, 2H), 2.91-2.75 (m, 1H), 2.29-2.18 (m, 6H), 1.79-1.58 (m, 10H), 1.55-1.48 (m, 2H), 1.36-1.22 (m, 5H), 1.20-1.14 (m, 5H), 1.08-0.99 (m, 1H). MS (ESI) m/z 872.4 [M+H]+.

The following examples in Table 24 were prepared according to Scheme VB—H using the procedures outlined in the synthesis of example 268.

TABLE 24

| | | | MS |
|---|---|---|---|
| Ex. | Structure | Name | (M + 1) |
| 271 Isomer A (Whelk o-02 CO2: 20% Me OH with 0.1% NH4O Ac) | 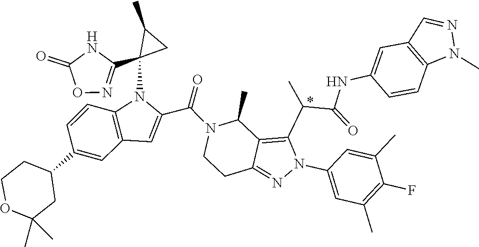 [SMILES: Cc1cc(-n2nc3c(c2C(C)C(=O)Nc2ccc4c(cnn4C)c2)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | (S)-2-((S)-5-(5-((S)-2,2- dimethyltetrahydro-2H-pyran-4- yl)-1-((1S,2S)-2-methyl-1-(5- oxo-4,5-dihydro-1,2,4- oxadiazol-3-yl)cyclopropyl)-1H- indole-2-carbonyl)-2-(4-fluoro- 3,5-dimethylphenyl)-4-methyl- 4,5,6,7-tetrahydro-2H- pyrazolo[4,3-c]pyridin-3-yl)-N- (1-methyl-1H-indazol-5- yl)propenamide or (R)-2-((S)-5- (5-((S)-2,2-dimethyltetrahydro- 2H-pyran-4-yl)-1-((1S,2S)-2- methyl-1-(5-oxo-4,5-dihydro- 1,2,4-oxadiazol-3- | 854.4 |
| | | yl)cyclopropyl)-1H-indole-2- | |
| | | carbonyl)-2-(4-fluoro-3,5- | |
| | | dimethylphenyl)-4-methyl- | |
| | | 4,5,6,7-tetrahydro-2H- | |
| | | pyrazolo[4,3-c]pyridin-3-yl)-N- | |
| | | (1-methyl-1H-indazol-5- | |
| | | yl)propanamide | |
| 271 Isomer B (Whelk o-02 CO2: 20% Me OH with 0.1% NH4O Ac) | 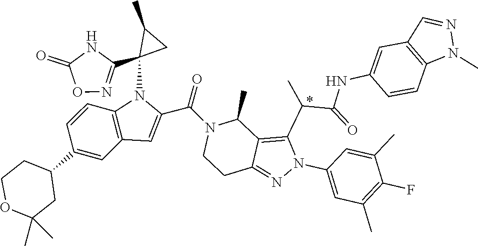 [SMILES: Cc1cc(-n2nc3c(c2C(C)C(=O)Nc2ccc4c(cnn4C)c2)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | (S)-2-((S)-5-(5-((S)-2,2- dimethyltetrahydro-2H-pyran-4- yl)-1-((1S,2S)-2-methyl-1-(5- oxo-4,5-dihydro-1,2,4- oxadiazol-3-yl)cyclopropyl)-1H- indole-2-carbonyl)-2-(4-fluoro- 3,5-dimethylphenyl)-4-methyl- 4,5,6,7-tetrahydro-2H- pyrazolo[4,3-c]pyridin-3-yl)-N- (1-methyl-1H-indazol-5- yl)propenamide or (R)-2-((S)-5- (5-((S)-2,2-dimethyltetrahydro- 2H-pyran-4-yl)-1-((1S,2S)-2- methyl-1-(5-oxo-4,5-dihydro- 1,2,4-oxadiazol-3- | 854.4 |
| | | yl)cyclopropyl)-1H-indole-2- | |
| | | carbonyl)-2-(4-fluoro-3,5- | |
| | | dimethylphenyl)-4-methyl- | |
| | | 4,5,6,7-tetrahydro-2H- | |
| | | pyrazolo[4,3-c]pyridin-3-yl)-N- | |
| | | (1-methyl-1H-indazol-5- | |
| | | yl)propanamide | |
| 273 Isomer A (Whelk o-02 CO2: 20% Me OH with 0.1% NH4O Ac) | 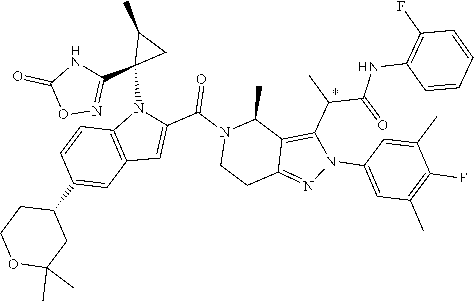 [SMILES: Cc1cc(-n2nc3c(c2C(C)C(=O)Nc2ccccc2F)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | (R)-2-((S)-5-(5-((S)-2,2- dimethyltetrahydro-2H-pyran-4- yl)-1-((1S,2S)-2-methyl-1-(5- oxo-4,5-dihydro-1,2,4- oxadiazol-3-yl)cyclopropyl)-1H- indole-2-carbonyl)-2-(4-fluoro- 3,5-dimethylphenyl)-4-methyl- 4,5,6,7-tetrahydro-2H- pyrazolo[4,3-c]pyridin-3-yl)-N- (2-fluorophenyl)propanamide or (S)-2-((S)-5-(5-((S)-2,2- dimethyltetrahydro-2H-pyran-4- yl)-1-((1S,2S)-2-methyl-1-(5- oxo-4,5-dihydro-1,2,4- oxadiazol-3-yl)cyclopropyl)-1H- indole-2-carbonyl)-2-(4-fluoro- 3,5-dimethylphenyl)-4-methyl- 4,5,6,7-tetrahydro-2H- | 818.4 |
| | | pyrazolo[4,3-c]pyridin-3-yl)-N- | |
| | | (2-fluorophenyl)propanamide | |
| 273 Isomer B (Whelk o-02 CO2: 20% Me OH with 0.1% NH4O Ac) | 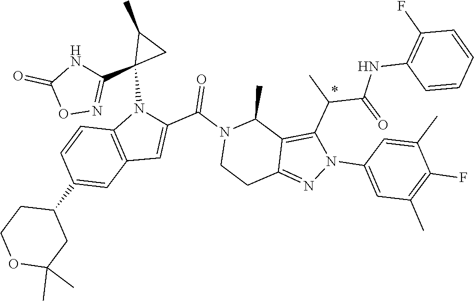 [SMILES: Cc1cc(-n2nc3c(c2C(C)C(=O)Nc2ccccc2F)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | (R)-2-((S)-5-(5-((S)-2,2- dimethyltetrahydro-2H-pyran-4- yl)-1-((1S,2S)-2-methyl-1-(5- oxo-4,5-dihydro-1,2,4- oxadiazol-3-yl)cyclopropyl)-1H- indole-2-carbonyl)-2-(4-fluoro- 3,5-dimethylphenyl)-4-methyl- 4,5,6,7-tetrahydro-2H- pyrazolo[4,3-c]pyridin-3-yl)-N- (2-fluorophenyl)propanamide or (S)-2-((S)-5-(5-((S)-2,2- dimethyltetrahydro-2H-pyran-4- yl)-1-((1S,2S)-2-methyl-1-(5- oxo-4,5-dihydro-1,2,4- oxadiazol-3-yl)cyclopropyl)-1H- indole-2-carbonyl)-2-(4-fluoro- 3,5-dimethylphenyl)-4-methyl- 4,5,6,7-tetrahydro-2H- | 818.4 |
| | | pyrazolo[4,3-c]pyridin-3-yl)-N- | |
| | | (2-fluorophenyl)propanamide | |
| 275 Isomer A (Prep- HPLC 70- 95% MeCN: H2O + 0.1% TFA) | 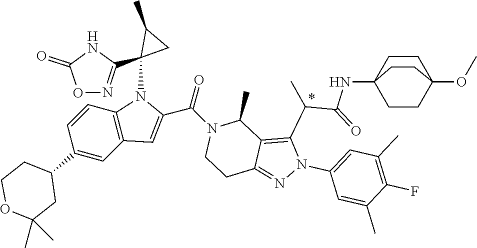 [SMILES: COC12CCC(NC(=O)C(C)c3c4c(nn3-c3cc(C)c(F)c(C)c3)CCN(C(=O)c3cc5cc([C@H]6CCOC(C)(C)C6)ccc5n3[C@@]3(c5noc(=O)[nH]5)C[C@@H]3C)[C@H]4C)(CC1)CC2] | (R)-2-((S)-5-(5-((S)-2,2- dimethyltetrahydro-2H-pyran-4- yl)-1-((1S,2S)-2-methyl-1-(5- oxo-4,5-dihydro-1,2,4- oxadiazol-3-yl)cyclopropyl)-1H- indole-2-carbonyl)-2-(4-fluoro- 3,5-dimethylphenyl)-4-methyl- 4,5,6,7-tetrahydro-2H- pyrazolo[4,3-c]pyridin-3-yl)-N- (4-methoxybicyclo[2.2.2]octan- 1-yl)propanamide or (S)-2-((S)- 5-(5-((S)-2,2- dimethyltetrahydro-2H-pyran-4- yl)-1-((1S,2S)-2-methyl-1-(5- oxo-4,5-dihydro-1,2,4- | 862.8 |
| | | oxadiazol-3-yl)cyclopropyl)-1H- | |
| | | indole-2-carbonyl)-2-(4-fluoro- | |
| | | 3,5-dimethylphenyl)-4-methyl- | |
| | | 4,5,6,7-tetrahydro-2H- | |
| | | pyrazolo[4,3-c]pyridin-3-yl)-N- | |
| | | (4-methoxybicyclo[2.2.2]octan- | |
| | | 1-yl)propanamide | |
| 275 Isomer B (Prep- HPLC 70- 95% MeCN: H2O + 0.1% TFA) | 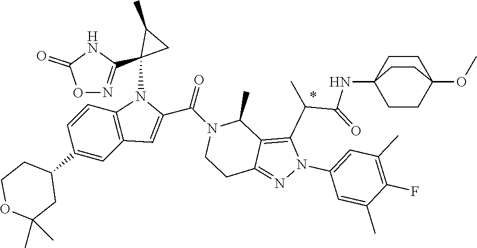 [SMILES: COC12CCC(NC(=O)C(C)c3c4c(nn3-c3cc(C)c(F)c(C)c3)CCN(C(=O)c3cc5cc([C@H]6CCOC(C)(C)C6)ccc5n3[C@@]3(c5noc(=O)[nH]5)C[C@@H]3C)[C@H]4C)(CC1)CC2] | (R)-2-((S)-5-(5-((S)-2,2- dimethyltetrahydro-2H-pyran-4- yl)-1-((1S,2S)-2-methyl-1-(5- oxo-4,5-dihydro-1,2,4- oxadiazol-3-yl)cyclopropyl)-1H- indole-2-carbonyl)-2-(4-fluoro- 3,5-dimethylphenyl)-4-methyl- 4,5,6,7-tetrahydro-2H- pyrazolo[4,3-c]pyridin-3-yl)-N- (4-methoxybicyclo[2.2.2]octan- 1-yl)propanamide or (S)-2-((S)- 5-(5-((S)-2,2- dimethyltetrahydro-2H-pyran-4- yl)-1-((1S,2S)-2-methyl-1-(5- oxo-4,5-dihydro-1,2,4- | 862.8 |
| | | oxadiazol-3-yl)cyclopropyl)-1H- | |
| | | indole-2-carbonyl)-2-(4-fluoro- | |
| | | 3,5-dimethylphenyl)-4-methyl- | |
| | | 4,5,6,7-tetrahydro-2H- | |
| | | pyrazolo[4,3-c]pyridin-3-yl)-N- | |
| | | (4-methoxybicyclo[2.2.2]octan- | |
| | | 1-yl)propanamide | |
| 277 Isomer A (Whelk o-02 CO2: 20% Me OH with 0.1% NH4O Ac) | 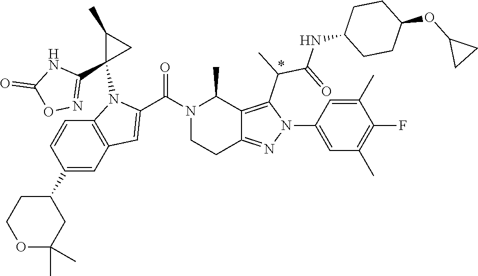 [SMILES: Cc1cc(-n2nc3c(c2C(C)C(=O)N[C@H]2CC[C@H](OC4CC4)CC2)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | (R)-N-((1r,4R)-4- cyclopropoxycyclohexyl)-2-((S)- 5-(5-((S)-2,2- dimethyltetrahydro-2H-pyran-4- yl)-1-((1S,2S)-2-methyl-1-(5- oxo-4,5-dihydro-1,2,4- oxadiazol-3-yl)cyclopropyl)-1H- indole-2-carbonyl)-2-(4-fluoro- 3,5-dimethylphenyl)-4-methyl- 4,5,6,7-tetrahydro-2H- pyrazolo[4,3-c]pyridin-3- yl)propanamide or (S)-N- ((1r,4S)-4- cyclopropoxycyclohexyl)-2-((S)- 5-(5-((S)-2,2- dimethyltetrahydro-2H-pyran-4- yl)-1-((1S,2S)-2-methyl-1-(5- | 862.5 |
| | | oxo-4,5-dihydro-1,2,4- | |
| | | oxadiazol-3-yl)cyclopropyl)-1H- | |
| | | indole-2-carbonyl)-2-(4-fluoro- | |
| | | 3,5-dimethylphenyl)-4-methyl- | |
| | | 4,5,6,7-tetrahydro-2H- | |
| | | pyrazolo[4,3-c]pyridin-3- | |
| | | yl)propanamide | |
| 277 Isomer B (Whelk o-02 CO2: 20% Me OH with 0.1% NH4O Ac) | 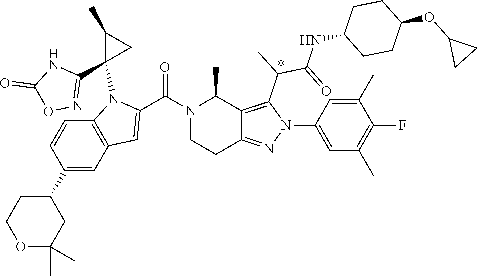 [SMILES: Cc1cc(-n2nc3c(c2C(C)C(=O)N[C@H]2CC[C@H](OC4CC4)CC2)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | (R)-N-((1r,4R)-4- cyclopropoxycyclohexyl)-2-((S)- 5-(5-((S)-2,2- dimethyltetrahydro-2H-pyran-4- yl)-1-((1S,2S)-2-methyl-1-(5- oxo-4,5-dihydro-1,2,4- oxadiazol-3-yl)cyclopropyl)-1H- indole-2-carbonyl)-2-(4-fluoro- 3,5-dimethylphenyl)-4-methyl- 4,5,6,7-tetrahydro-2H- pyrazolo[4,3-c]pyridin-3- yl)propanamide or (S)-N- ((1r,4S)-4- cyclopropoxycyclohexyl)-2-((S)- 5-(5-((S)-2,2- dimethyltetrahydro-2H-pyran-4- yl)-1-((1S,2S)-2-methyl-1-(5- | 862.5 |
| | | oxo-4,5-dihydro-1,2,4- | |
| | | oxadiazol-3-yl)cyclopropyl)-1H- | |
| | | indole-2-carbonyl)-2-(4-fluoro- | |
| | | 3,5-dimethylphenyl)-4-methyl- | |
| | | 4,5,6,7-tetrahydro-2H- | |
| | | pyrazolo[4,3-c]pyridin-3- | |
| | | yl)propanamide | |
| 279 Isomer A (Whelk o-02 CO2: 25% Me OH with 0.1% NH4O Ac) | 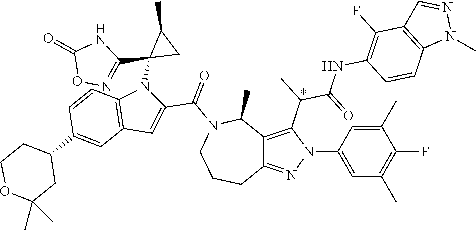 [SMILES: Cc1cc(-n2nc3c(c2C(C)C(=O)Nc2ccc4c(cnn4C)c2F)[C@H](C)N(C(=O)c2cc4cc([C@H]5CCOC(C)(C)C5)ccc4n2[C@@]2(c4noc(=O)[nH]4)C[C@@H]2C)CCC3)cc(C)c1F] | (R)-2-((S)-5-(5-((S)-2,2- dimethyltetrahydro-2H-pyran-4- yl)-1-((1S,2S)-2-methyl-1-(5- oxo-4,5-dihydro-1,2,4- oxadiazol-3-yl)cyclopropyl)-1H- indole-2-carbonyl)-2-(4-fluoro- 3,5-dimethylphenyl)-4-methyl- 2,4,5,6,7,8- hexahydropyrazolo[4,3- c]azepin-3-yl)-N-(4-fluoro-1- methyl-1H-indazol-5- yl)propanamide or (S)-2-((S)-5- (5-((S)-2,2-dimethyltetrahydro- 2H-pyran-4-yl)-1-((1S,2S)-2- | 886.8 |
| | | methyl-1-(5-oxo-4,5-dihydro- | |
| | | 1,2,4-oxadiazol-3- | |
| | | yl)cyclopropyl)-1H-indole-2- | |
| | | carbonyl)-2-(4-fluoro-3,5- | |
| | | dimethylphenyl)-4-methyl- | |
| | | 2,4,5,6,7,8- | |
| | | hexahydropyrazolo[4,3- | |
| | | c]azepin-3-yl)-N-(4-fluoro-1- | |
| | | methyl-1H-indazol-5- | |
| | | yl)propanamide | |
| 279 Isomer B (Whelk o-02 CO2: 25% Me OH with 0.1% NH4O Ac) | 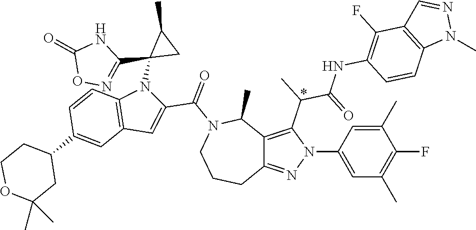 [SMILES: Cc1cc(-n2nc3c(c2C(C)C(=O)Nc2ccc4c(cnn4C)c2F)[C@H](C)N(C(=O)c2cc4cc([C@H]5CCOC(C)(C)C5)ccc4n2[C@@]2(c4noc(=O)[nH]4)C[C@@H]2C)CCC3)cc(C)c1F] | (R)-2-((S)-5-(5-((S)-2,2- dimethyltetrahydro-2H-pyran-4- yl)-1-((1S,2S)-2-methyl-1-(5- oxo-4,5-dihydro-1,2,4- oxadiazol-3-yl)cyclopropyl)-1H- indole-2-carbonyl)-2-(4-fluoro- 3,5-dimethylphenyl)-4-methyl- 2,4,5,6,7,8- hexahydropyrazolo[4,3- c]azepin-3-yl)-N-(4-fluoro-1- methyl-1H-indazol-5- yl)propanamide or (S)-2-((S)-5- (5-((S)-2,2-dimethyltetrahydro- 2H-pyran-4-yl)-1-((1S,2S)-2- | 886.8 |
| | | methyl-1-(5-oxo-4,5-dihydro- | |
| | | 1,2,4-oxadiazol-3- | |
| | | yl)cyclopropyl)-1H-indole-2- | |
| | | carbonyl)-2-(4-fluoro-3,5- | |
| | | dimethylphenyl)-4-methyl- | |
| | | 2,4,5,6,7,8- | |
| | | hexahydropyrazolo[4,3- | |
| | | c]azepin-3-yl)-N-(4-fluoro-1- | |
| | | methyl-1H-indazol-5- | |
| | | yl)propanamide | |
| 333 Isomer A (Cel- 3, CO2: 25- 50% MeOH with 20 mM NH3) | 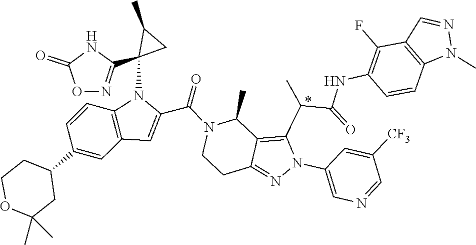 [SMILES: CC(C(=O)Nc1ccc2c(cnn2C)c1F)c1c2c(nn1-c1cncc(C(F)(F)F)c1)CCN(C(=O)c1cc3cc([C@H]4CCOC(C)(C)C4)ccc3n1[C@@]1(c3noc(=O)[nH]3)C[C@@H]1C)[C@H]2C] | (R)-2-((S)-5-(5-((S)-2,2- dimethyltetrahydro-2H-pyran-4- yl)-1-((1S,2S)-2-methyl-1-(5- oxo-4,5-dihydro-1,2,4- oxadiazol-3-yl)cyclopropyl)-1H- indole-2-carbonyl)-4-methyl-2- (5-(trifluoromethyl)pyridin-3- yl)-4,5,6,7-tetrahydro-2H- pyrazolo[4,3-c]pyridin-3-yl)-N- (4-fluoro-1-methyl-1H-indazol- 5-yl)propenamide or (S)-2-((S)- 5-(5-((S)-2,2- dimethyltetrahydro-2H-pyran-4- yl)-1-((1S,2S)-2-methyl-1-(5- oxo-4,5-dihydro-1,2,4- | 895.2 |
| | | oxadiazol-3-yl)cyclopropyl)-1H- | |
| | | indole-2-carbonyl)-4-methyl-2- | |
| | | (5-(trifluoromethyl)pyridin-3- | |
| | | yl)-4,5,6,7-tetrahydro-2H- | |
| | | pyrazolo[4,3-c]pyridin-3-yl)-N- | |
| | | (4-fluoro-1-methyl-1H-indazol- | |
| | | 5-yl)propanamide | |
| 333 Isomer B (Cel- 3, CO2: 25- 50% MeOH with 20 mM NH3) | 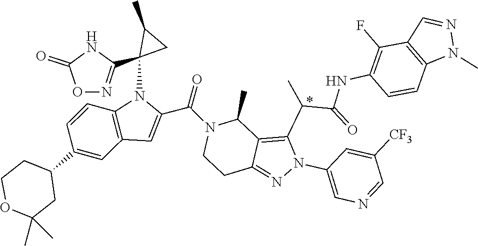 [SMILES: CC(C(=O)Nc1ccc2c(cnn2C)c1F)c1c2c(nn1-c1cncc(C(F)(F)F)c1)CCN(C(=O)c1cc3cc([C@H]4CCOC(C)(C)C4)ccc3n1[C@@]1(c3noc(=O)[nH]3)C[C@@H]1C)[C@H]2C] | (R)-2-((S)-5-(5-((S)-2,2- dimethyltetrahydro-2H-pyran-4- yl)-1-((1S,2S)-2-methyl-1-(5- oxo-4,5-dihydro-1,2,4- oxadiazol-3-yl)cyclopropyl)-1H- indole-2-carbonyl)-4-methyl-2- (5-(trifluoromethyl)pyridin-3- yl)-4,5,6,7-tetrahydro-2H- pyrazolo[4,3-c]pyridin-3-yl)-N- (4-fluoro-1-methyl-1H-indazol- 5-yl)propenamide or (S)-2-((S)- 5-(5-((S)-2,2- dimethyltetrahydro-2H-pyran-4- yl)-1-((1S,2S)-2-methyl-1-(5- oxo-4,5-dihydro-1,2,4- | 895.2 |
| | | oxadiazol-3-yl)cyclopropyl)-1H- | |
| | | indole-2-carbonyl)-4-methyl-2- | |
| | | (5-(trifluoromethyl)pyridin-3- | |
| | | yl)-4,5,6,7-tetrahydro-2H- | |
| | | pyrazolo[4,3-c]pyridin-3-yl)-N- | |
| | | (4-fluoro-1-methyl-1H-indazol- | |
| | | 5-yl)propanamide | |
| 334 Isomer A (Cel- 3, CO2: 25- 50% MeOH with 20 mM NH3) | 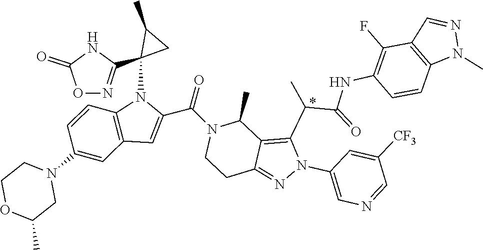 | (R)-N-(4-fluoro-1-methyl-1H- indazol-5-yl)-2-((S)-4-methyl-5- (1-((1S,2S)-2-methyl-1-(5-oxo- 4,5-dihydro-1,2,4-oxadiazol-3- yl)cyclopropyl)-5-((S)-2- methylmorpholino)-1H-indole-2- carbonyl)-2-(5- (trifluoromethyl)pyridin-3-yl)- 4,5,6,7-tetrahydro-2H- pyrazolo[4,3-c]pyridin-3- yl)propenamide or (S)-N-(4- fluoro-1-methyl-1H-indazol-5- yl)-2-((S)-4-methyl-5-(1- ((1S,2S)-2-methyl-1-(5-oxo-4,5- dihydro-1,2,4-oxadiazol-3- | 910.2 |
| | | yl)cyclopropyl)-5-((S)-2- | |
| | | methylmorpholino)-1H-indole-2- | |
| | | carbonyl)-2-(5- | |
| | | (trifluoromethyl)pyridin-3-yl)- | |
| | | 4,5,6,7-tetrahydro-2H- | |
| | | pyrazolo[4,3-c]pyridin-3- | |
| | | yl)propanamide | |
| 334 Isomer B (Cel- 3, CO2: 25- 50% MeOH with 20 mM NH3) | 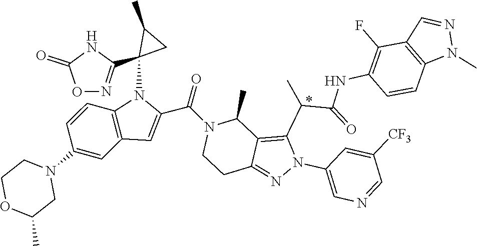 | (R)-N-(4-fluoro-1-methyl-1H- indazol-5-yl)-2-((S)-4-methyl-5- (1-((1S,2S)-2-methyl-1-(5-oxo- 4,5-dihydro-1,2,4-oxadiazol-3- yl)cyclopropyl)-5-((S)-2- methylmorpholino)-1H-indole-2- carbonyl)-2-(5- (trifluoromethyl)pyridin-3-yl)- 4,5,6,7-tetrahydro-2H- pyrazolo[4,3-c]pyridin-3- yl)propenamide or (S)-N-(4- fluoro-1-methyl-1H-indazol-5- yl)-2-((S)-4-methyl-5-(1- ((1S,2S)-2-methyl-1-(5-oxo-4,5- dihydro-1,2,4-oxadiazol-3- | 910.2 |
| | | yl)cyclopropyl)-5-((S)-2- | |
| | | methylmorpholino)-1H-indole-2- | |
| | | carbonyl)-2-(5- | |
| | | (trifluoromethyl)pyridin-3-yl)- | |
| | | 4,5,6,7-tetrahydro-2H- | |
| | | pyrazolo[4,3-c]pyridin-3- | |
| | | yl)propanamide | |

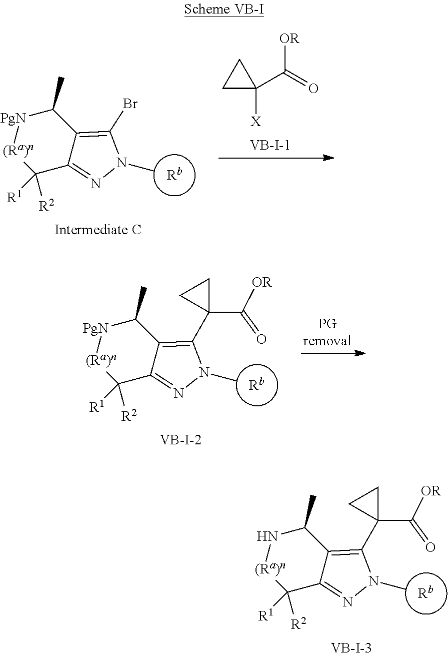

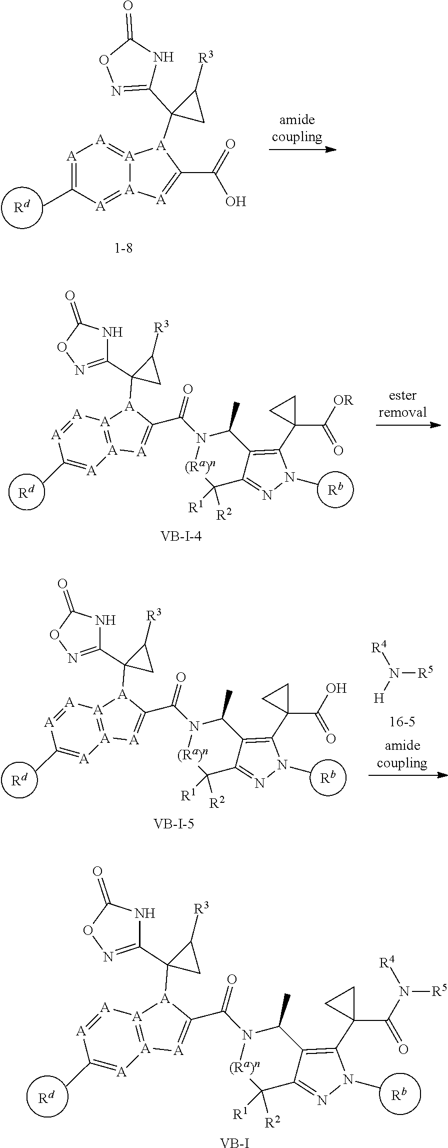

Compounds of formula VB—I are prepared from Intermediate C by coupling with metal enolates such as zinc enolates (16-VB—I-1, depicted arbitrarily as one possible tautomer) via transition metal catalyzed cross-coupling conditions (e.g., Pd—or Cu-catalysis) to provide VB—I-2. Removal of the protecting group (e.g., acidic conditions) of VB—I-2 provides amine VB—I-3 that is then coupled with carboxylic acid 1-8 to provide compounds of formula VB—I-4. Removal of the ester (e.g., basic aqueous conditions) of VB—I-4 furnishes carboxylic acid VB—I-5. Subsequently, 16-5 is coupled with VB—I-5 to provide VB—I. Alternatively, the order of steps for some examples may be varied to facilitate the syntheses of the title compounds of formula VB—I.

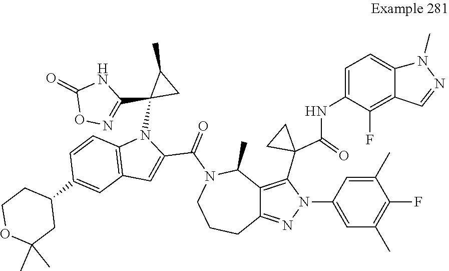

### 1-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-2,4,5,6,7,8-hexahydropyrazolo[4,3-c]azepin-3-yl)-N-(4-fluoro-1-methyl-1H-indazol-5-yl)cyclopropane-1-carboxamide

### Step 1: tert-butyl (S)-2-(4-fluoro-3,5-dimethylphenyl)-3-(1-(methoxycarbonyl) cyclopropyl)-4-methyl-2,6,7,8-tetrahydropyrazolo[4,3-c]azepine-5(4H)-carboxylate

To a mixture of tert-butyl (S)-3-bromo-2-(4-fluoro-3,5-dimethylphenyl)-4-methyl-2,6,7,8-tetrahydropyrazolo[4,3-c]azepine-5(4H)-carboxylate (5.00 g, 11.1 mmol, 1 equiv) and Bis(tri-tert-butylphosphine)palladium(0) (565 mg, 1.11 mmol, 0.1 equiv) was added a solution of (1-(methoxycarbonyl)cyclopropyl)zinc(II) bromide (0.35 M in THF, 41.1 mL, 14.4 mmol, 1.3 equiv) under argon atmosphere, and the resulting solution was heated to 60° C. for 18 h. The mixture was then cooled, diluted with EtOAc (50 mL), and sat. aq. NaHCO₃ (50 mL) was slowly added to form a precipitate. The mixture was filtered over Celite® and the resulting biphasic mixture was extracted with EtOAc (2×50 mL). The combined organic layers were dried over Na₂SO₄, concentrated under reduced pressure, and purified by flash column chromatography (0 to 100% EtOAc/hexanes) to give the title compound. MS (ESI) m/z: 472.2 [M+H]⁺

### Step 2: methyl 1-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-2,4,5,6,7,8-hexahydropyrazolo[4,3-c]azepin-3-yl)cyclopropane-1-carboxylate

To tert-butyl (S)-2-(4-fluoro-3,5-dimethylphenyl)-3-(1-(methoxycarbonyl)cyclopropyl)-4-methyl-2,6,7,8-tetrahydropyrazolo[4,3-c]azepine-5(4H)-carboxylate (0.80 g, 1.7 mmol, 1 equiv) was added HCl (4 M in dioxane, 8.4 mL, 34 mmol, 20 equiv), and the mixture was stirred for 30 min before being concentrated under reduced pressure. To the resulting foam was added Intermediate AF1 (768 mg, 1.86 mmol, 1.1 equiv), HATU (709 mg, 1.86 mmol, 1.1 equiv), DMF (5.5 mL, 0.3 M), and DIPEA (2.1 mL, 12 mmol, 7 equiv), and the mixture was stirred for 18 h. Then 10% aq. LiCl (20 mL) and EtOAc (20 mL) were added and the mixture extracted with EtOAc (3×30 mL). The combined organic layers were washed with brine (10 mL), dried over Na₂SO₄, and concentrated under reduced pressure. The crude mixture was purified by flash column chromatography (0-100% EtOAc/hexanes) to give the title compound. MS (ESI) m/z: 765.6 [M+H]⁺

### Step 3: 1-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-2,4,5,6,7,8-hexahydropyrazolo[4,3-c]azepin-3-yl)cyclopropane-1-carboxylic acid

To a solution of methyl 1-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-2,4,5,6,7,8-hexahydropyrazolo[4,3-c]azepin-3-yl)cyclopropane-1-carboxylate in THF (10 mL), water (10 mL), and MeOH (10 mL) was added NaOH (10 M, 3.4 mL, 34 mmol, 20 equiv), and the mixture was stirred at 60° C. for 18 h. Then the solution was cooled to ambient temperature and conc. HCl (2 mL) was added dropwise to bring the pH to 3. Aq. LiCl (10%, 30 mL) and EtOAc (30 mL) were then added, and the biphasic mixture was extracted with EtOAc (2×40 mL). The combined organic layers were dried over Na₂SO₄ and concentrated under reduced pressure. The crude mixture was purified by reverse phase C18 (100 g, 0 to 100% MeCN/water 0.1% formic acid) to give the title compound. MS (ESI) m/z: 751.2 [M+H]⁺

### Step 4: 1-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-2,4,5,6,7,8-hexahydropyrazolo[4,3-c]azepin-3-yl)-N-(4-fluoro-1-methyl-1H-indazol-5-yl)cyclopropane-1-carboxamide

To a solution of 1-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-2,4,5,6,7,8-hexahydropyrazolo[4,3-c]azepin-3-yl)cyclopropane-1-carboxylic acid (50.0 mg, 66.6 mol, 1 equiv), 1-methyl-1H-imidazole (32 L, 33 mg, 0.40 mmol, 6 equiv), and 4-fluoro-1-methyl-1H-indazol-5-amine (33.0 mg, 0.20 mmol, 3 equiv) in MeCN (6.6 mL, 0.01 M) was added TCFH (37.4 mg, 133 mol, 2 equiv) as a solution in MeCN (533 μL, 0.25 M). The mixture was stirred for 18 h, then concentrated and purified by reverse phase column chromatography on ACCQ-Prep C18, 22 mL/min, 0% to 100% MeCN/H₂O 0.1% formic acid to give the title compound. MS (ESI) m/z: 898.4 [M+H]⁺. ¹H NMR (500 MHz, CD₃CN) δ 11.32 (s, 1H), 8.04 & 8.02 (rotameric, 1H), 7.94 & 7.82 (rotameric, s, 1H), 7.54 (s, 1H), 7.50-7.45 & 7.41-7.36 (rotameric, m, 2H), 7.44 (d, J=4.2 Hz, 2H), 7.32-7.16 (in, 811), 7.13 (d, J=6.2 Hz, 211), 6.88 & 6.62 (rotameric, s, 1H), 6.61-6.54 & 6.14-6.02 (rotameric, m, 1H), 4.63 & 4.18 (rotameric, d, J=15 Hz, 1H), 4.03 & 3.93 (rotameric, s, 3H), 3.85-3.69 (in, 7H), 3.69-3.61 (in, 1H), 3.13-2.93 (in, 8H), 2.33 & 2.27 (rotameric, s, 6H), 2.09-1.97 (in, 5H), 1.87 (d, J=7.0 Hz, 3H), 1.82-1.78 (in, 2H), 1.68 (q, J=12.8 Hz, 12H), 1.60 (ddd, J=14.8, 10.8, 5.7 Hz, 5H), 1.57-1.49 (m, 3H), 1.27 (s, 7H), 1.18 (d, J=6.1 Hz, 11H), 1.10 (dd, J=9.5, 6.4 Hz, 1H), 0.99 (d, J=6.2 Hz, 3H), 0.93 (s, 1H), 0.77 (s, 1H), 0.48 (s, 1H), 0.20 (s, 1H).

The following examples in Table 25 were prepared using the procedures outlined in the synthesis of example 281. The order of steps for some examples may be varied from Scheme VB-1 to facilitate the syntheses of the title compounds of formula (XVI).

TABLE 25

| | | | MS |
|---|---|---|---|
| Ex. | Structure | Name | (M + 1) |
| 282 | 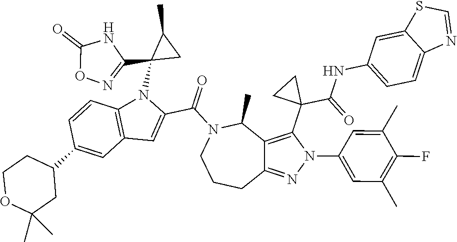 | N-(1,3-benzothiazol-3-ium-6- yl)-1-[(4S)-5-[5-[(4S)-2,2- dimethyltetrahydropyran-4- yl]-1-[(1S,2S)-2-methyl-1- (5-oxo-4H-1,2,4-oxadiazol-3- yl)cyclopropyl]indole-2- carbonyl]-2-(4-fluoro-3,5- dimethyl-phenyl)-4-methyl- 4,6,7,8-tetrahydropyrazolo [4,3-c]azepin-3-yl]cyclopro- panecarboxamide; 2,2,2- trifluoroacetate | 883.9 |
| 283 | 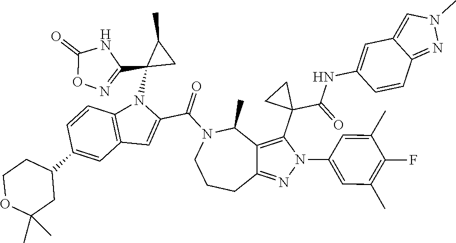 | 1-[(4S)-5-[5-[(4S)-2,2- dimethyltetrahydropyran-4- yl]-1-[(1S,2S)-2-methyl-1- (5-oxo-4H-1,2,4-oxadiazol- 3-yl)cyclopropyl]indole-2- carbonyl]-2-(4-fluoro-3,5- dimethyl-phenyl)-4-methyl- 4,6,7,8-tetrahydropyrazolo [4,3-c]azepin-1-ium-3-yl]- N-(2-methylindazol-5-yl) cyclopropanecarboxamide formate | 881.4 |
| 284 | 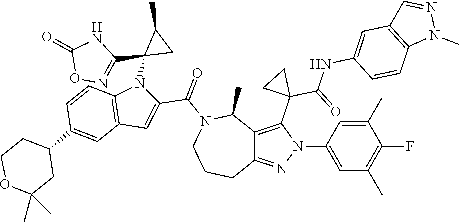 [SMILES: Cc1cc(-n2nc3c(c2C2(C(=O)Nc4ccc5c(cnn5C)c4)CC2)[C@H](C)N(C(=O)c2cc4cc([C@H]5CCOC(C)(C)C5)ccc4n2[C@@]2(c4noc(=O)[nH]4)C[C@@H]2C)CCC3)cc(C)c1F] | 1-[(4S)-5-[5-[(4S)-2,2- dimethyltetrahydropyran-4- yl]-1-[(1S,2S)-2-methyl-1- (5-oxo-4H-1,2,4-oxadiazol- 3-yl)cyclopropyl]indole-2- carbonyl]-2-(4-fluoro-3,5- dimethyl-phenyl)-4-methyl- 4,6,7,8-tetrahydropyrazolo [4,3-c]azepin-1-ium-3-yl]- N-(1-methylindazol-5-yl) cyclopropanecarboxamide formate | 880.6 |
| 285 | 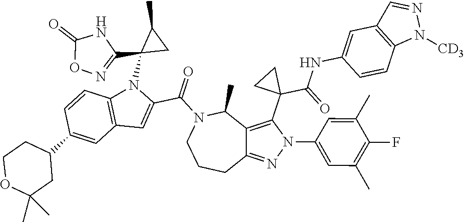 | 1-[(4S)-5-[5-[(4S)-2,2- dimethyltetrahydropyran- 4-yl]-1-[(1S,2S)-2-methyl- 1-(5-oxo-4H-1,2,4-oxadiazol- 3-yl)cyclopropyl]indole-2- carbonyl]-2-(4-fluoro-3,5- dimethyl-phenyl)-4-methyl- 4,6,7,8-tetrahydropyrazolo [4,3-c]azepin-1-ium-3-yl]- N-[1-(trideuteriomethyl) indazol-5-yl]cyclopropane- carboxamide formate | 883.6 |
| 286 | 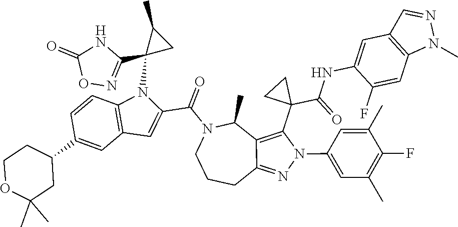 [SMILES: Cc1cc(-n2nc3c(c2C2(C(=O)Nc4cc5cnn(C)c5cc4F)CC2)[C@H](C)N(C(=O)c2cc4cc([C@H]5CCOC(C)(C)C5)ccc4n2[C@@]2(c4noc(=O)[nH]4)C[C@@H]2C)CCC3)cc(C)c1F] | 1-[(4S)-5-[5-[(4S)-2,2- dimethyltetrahydropyran- 4-yl]-1-[(1S,2S)-2-methyl- 1-(5-oxo-4H-1,2,4-oxadiazol- 3-yl)cyclopropyl]indole-2- carbonyl]-2-(4-fluoro-3,5- dimethyl-phenyl)-4-methyl- 4,6,7,8-tetrahydropyrazolo [4,3-c]azepin-1-ium-3-yl]- N-(6-fluoro-1-methyl- indazol-5-yl)cyclopropane- carboxamide formate | 898.6 |
| 287 | 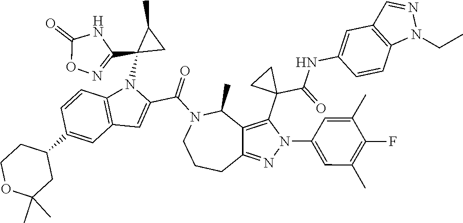 [SMILES: CCn1ncc2cc(NC(=O)C3(c4c5c(nn4-c4cc(C)c(F)c(C)c4)CCCN(C(=O)c4cc6cc([C@H]7CCOC(C)(C)C7)ccc6n4[C@@]4(c6noc(=O)[nH]6)C[C@@H]4C)[C@H]5C)CC3)ccc21] | 1-[(4S)-5-[5-[(4S)-2,2- dimethyltetrahydropyran- 4-yl]-1-[(1S,2S)-2-methyl- 1-(5-oxo-4H-1,2,4- oxadiazol-3-yl)cyclopropyl] indole-2-carbonyl]-2-(4- fluoro-3,5-dimethyl- phenyl)-4-methyl-4,6,7,8- tetrahydropyrazolo [4,3-c] azepin-1-ium-3-yl]-N-(1- ethylindazol-5-yl)cyclopro- panecarboxamide; 2,2,2-trifluoroacetate | 894.1 |
| 288 | 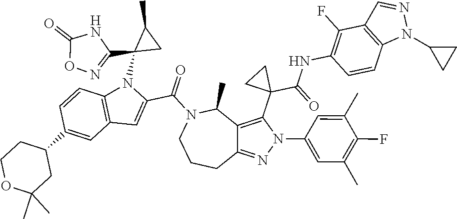 [SMILES: Cc1cc(-n2nc3c(c2C2(C(=O)Nc4ccc5c(cnn5C5CC5)c4F)CC2)[C@H](C)N(C(=O)c2cc4cc([C@H]5CCOC(C)(C)C5)ccc4n2[C@@]2(c4noc(=O)[nH]4)C[C@@H]2C)CCC3)cc(C)c1F] | N-(1-cyclopropyl-4-fluoro- indazol-5-yl)-1-[(4S)-5- [5-[(4S)-2,2-dimethyltetra- hydropyran-4-yl]-1-[(1S,2S)- 2-methyl-1-(5-oxo-4H-1,2,4- oxadiazol-3-yl)cyclopropyl] indole-2-carbonyl]-2-(4- fluoro-3,5-dimethyl-phenyl)- 4-methyl-4,6,7,8-tetrahydro- pyrazolo [4,3-c]azepin-1- ium-3-yl]cyclopropane- carboxamide formate | 924.6 |
| 289 | 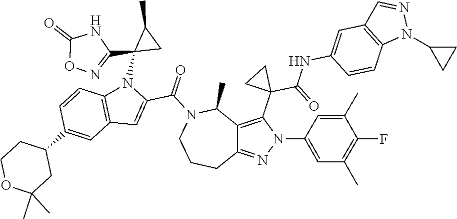 [SMILES: Cc1cc(-n2nc3c(c2C2(C(=O)Nc4ccc5c(cnn5C5CC5)c4)CC2)[C@H](C)N(C(=O)c2cc4cc([C@H]5CCOC(C)(C)C5)ccc4n2[C@@]2(c4noc(=O)[nH]4)C[C@@H]2C)CCC3)cc(C)c1F] | N-(1-cyclopropylindazol- 5-yl)-1-[(4S)-5-[5-[(4S)- 2,2-dimethyltetrahydro- pyran-4-yl]-1-[(1S,2S)- 2-methyl-1-(5-oxo-4H- 1,2,4-oxadiazol-3-yl) cyclopropyl]indole-2- carbonyl]-2-(4-fluoro- 3,5-dimethyl-phenyl)- 4-methyl-4,6,7,8-tetra- hydropyrazolo[4,3-c] azepin-1-ium-3-yl] cyclopropanecarboxamide formate | 906.6 |
| 290 | 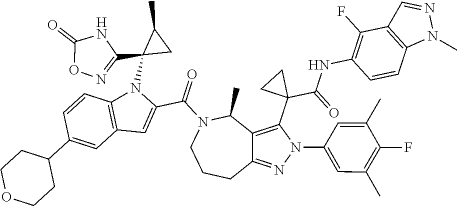 [SMILES: Cc1cc(-n2nc3c(c2C2(C(=O)Nc4ccc5c(cnn5C)c4F)CC2)[C@H](C)N(C(=O)c2cc4cc(C5CCOCC5)ccc4n2[C@@]2(c4noc(=O)[nH]4)C[C@@H]2C)CCC3)cc(C)c1F] | 1-[(4S)-2-(4-fluoro-3,5- dimethyl-phenyl)-4-methyl- 5-[1-[(1S,2S)-2-methyl-1- (5-oxo-4H-1,2,4-oxadiazol- 3-yl)cyclopropyl]-5-tetra- hydropyran-4-yl-indole-2- carbonyl]-4,6,7,8-tetra- hydropyrazolo[4,3-c] azepin-1-ium-3-yl]-N- (4-fluoro-1-methyl- indazol-5-yl)cyclopro- panecarboxamide formate | 870.4 |
| 291 | 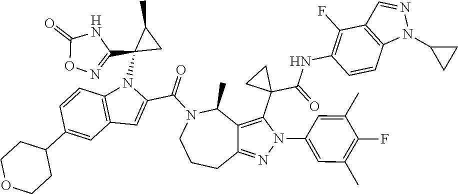 [SMILES: Cc1cc(-n2nc3c(c2C2(C(=O)Nc4ccc5c(cnn5C5CC5)c4F)CC2)[C@H](C)N(C(=O)c2cc4cc(C5CCOCC5)ccc4n2[C@@]2(c4noc(=O)[nH]4)C[C@@H]2C)CCC3)cc(C)c1F] | N-(1-cyclopropyl-4-fluoro- indazol-5-yl)-1-[(4S)-2- (4-fluoro-3,5-dimethyl- phenyl)-4-methyl-5-[1- [(1S,2S)-2-methyl-1-(5- oxo-4H-1,2,4-oxadiazol- 3-yl)cyclopropyl]-5-tetra- hydropyran-4-yl-indole- 2-carbonyl]-4,6,7,8- tetrahydropyrazolo [4,3-c]azepin-1-ium-3- yl]cyclopropane- | 896.4 |
| | | carboxamide formate | |
| 292 | 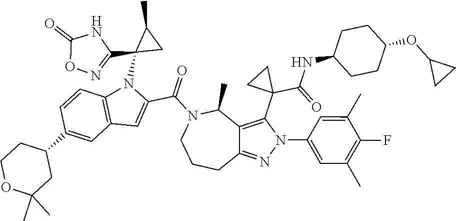 [SMILES: Cc1cc(-n2nc3c(c2C2(C(=O)N[C@H]4CC[C@H](OC5CC5)CC4)CC2)[C@H](C)N(C(=O)c2cc4cc([C@H]5CCOC(C)(C)C5)ccc4n2[C@@]2(c4noc(=O)[nH]4)C[C@@H]2C)CCC3)cc(C)c1F] | N-[4-(cyclopropoxy) cyclohexyl]-1-[(4S)-5- [5-[(4S)-2,2-dimethyl- tetrahydropyran-4-yl]- 1-[(1S,2S)-2-methyl-1- (5-oxo-4H-1,2,4- oxadiazol-3-yl)cyclopropyl] indole-2-carbonyl]-2-(4- fluoro-3,5-dimethyl- phenyl)-4-methyl-4,6,7,8- tetrahydropyrazolo[4,3-c] azepin-1-ium-3-yl]cyclo- propanecarboxamide; 2,2,2-trifluoroacetate | 888.4 |
| 293 | 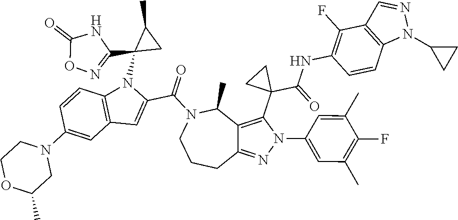 [SMILES: Cc1cc(-n2nc3c(c2C2(C(=O)Nc4ccc5c(cnn5C5CC5)c4F)CC2)[C@H](C)N(C(=O)c2cc4cc(N5CCO[C@@H](C)C5)ccc4n2[C@@]2(c4noc(=O)[nH]4)C[C@@H]2C)CCC3)cc(C)c1F] | N-(1-cyclopropyl-4- fluoro-indazol-5-yl)-1- [(4S)-2-(4-fluoro-3,5- dimethyl-phenyl)-4- methyl-5-[5-[(2S)-2- methylmorpholin-4- ium-4-yl]-1-[(1S,2S)- 2-methyl-1-(5-oxo-4H- 1,2,4-oxadiazol-3-yl) cyclopropyl]indole-2- carbonyl]- 4,6,7,8-tetrahydro- pyrazolo[4,3-c]azepin- 3-yl]cyclopropane- | 911.4 |
| | | carboxamide formate | |
| 294 | 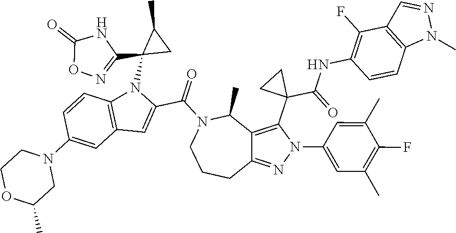 [SMILES: Cc1cc(-n2nc3c(c2C2(C(=O)Nc4ccc5c(cnn5C)c4F)CC2)[C@H](C)N(C(=O)c2cc4cc(N5CCO[C@@H](C)C5)ccc4n2[C@@]2(c4noc(=O)[nH]4)C[C@@H]2C)CCC3)cc(C)c1F] | 1-[(4S)-2-(4-fluoro- 3,5-dimethyl-phenyl)-4- methyl-5-[5-[(2S)-2- methylmorpholin-4-ium- 4-yl]-1-[(1S,2S)-2-methyl- 1-(5-oxo-4H-1,2,4-oxadiazol- 3-yl)cyclopropyl]indole-2- carbonyl]-4,6,7,8-tetrahydro- pyrazolo[4,3-c]azepin-3- yl]-N-(4-fluoro-1-methyl- indazol-5-yl)cyclopropane- carboxamide formate | 885.4 |
| 295 | 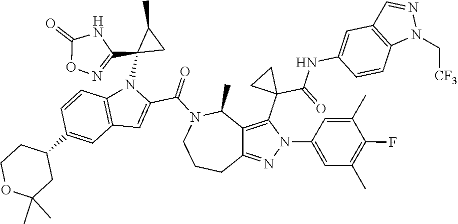 [SMILES: Cc1cc(-n2nc3c(c2C2(C(=O)Nc4ccc5c(cnn5CC(F)(F)F)c4)CC2)[C@H](C)N(C(=O)c2cc4cc([C@H]5CCOC(C)(C)C5)ccc4n2[C@@]2(c4noc(=O)[nH]4)C[C@@H]2C)CCC3)cc(C)c1F] | 1-[(4S)-5-[5-[(4S)-2,2- dimethyltetrahydropyran- 4-yl]-1-[(1S,2S)-2-methyl- 1-(5-oxo-4H-1,2,4-oxadiazol- 3-yl)cyclopropyl]indole-2- carbonyl]-2-(4-fluoro-3,5- dimethyl-phenyl)-4-methyl- 4,6,7,8-tetrahydropyrazolo [4,3-c]azepin-1-ium-3-yl]- N-[1-(2,2,2-trifluoroethyl) indazol-5-yl]cyclopropane- carboxamide formate | 948.6 |
| 296 | 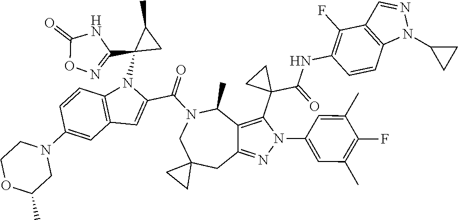 [SMILES: Cc1cc(-n2nc3c(c2C2(C(=O)Nc4ccc5c(cnn5C5CC5)c4F)CC2)[C@H](C)N(C(=O)c2cc4cc(N5CCO[C@@H](C)C5)ccc4n2[C@@]2(c4noc(=O)[nH]4)C[C@@H]2C)CC2(CC2)C3)cc(C)c1F] | N-(1-cyclopropyl-4- fluoro-indazol-5-yl)-1- [(4S)-2-(4-fluoro-3,5- dimethyl-phenyl)-4- methyl-5-[5-[(2S)-2- methylmorpholin-4- ium-4-yl]-1-[(1S,2S)- 2-methyl-1-(5-oxo-4H- 1,2,4-oxadiazol-3-yl) cyclopropyl]indole-2- carbonyl]spiro[6,8- dihydro-4H-pyrazolo [4,3-c]azepine-7,1′- | 937.6 |
| | | cyclopropane]-3-yl] | |
| | | cyclopropane- | |
| | | carboxamide formate | |
| 297 | 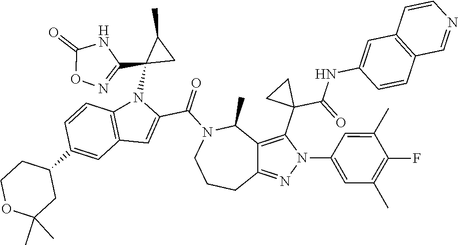 [SMILES: Cc1cc(-n2nc3c(c2C2(C(=O)Nc4ccc5cnccc5c4)CC2)[C@H](C)N(C(=O)c2cc4cc([C@H]5CCOC(C)(C)C5)ccc4n2[C@@]2(c4noc(=O)[nH]4)C[C@@H]2C)CCC3)cc(C)c1F] | 1-[(4S)-5-[5-[(4S)-2,2- dimethyltetrahydropyran- 4-yl]-1-[(1S,2S)-2-methyl- 1-(5-oxo-4H-1,2,4- oxadiazol-3-yl)cyclo- propyl]indole-2-carbonyl]- 2-(4-fluoro-3,5-dimethyl- phenyl)-4-methyl-4,6,7,8- tetrahydropyrazolo[4,3-c] azepin-3-yl]-N-isoquinolin- 2-ium-6-yl-cyclopropane- carboxamide formate | 877.4 |
| 298 | 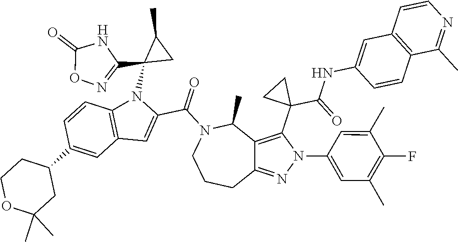 [SMILES: Cc1cc(-n2nc3c(c2C2(C(=O)Nc4ccc5c(C)nccc5c4)CC2)[C@H](C)N(C(=O)c2cc4cc([C@H]5CCOC(C)(C)C5)ccc4n2[C@@]2(c4noc(=O)[nH]4)C[C@@H]2C)CCC3)cc(C)c1F] | 1-[(4S)-5-[5-[(4S)-2,2- dimethyltetrahydropyran- 4-yl]-1-[(1S,2S)-2-methyl- 1-(5-oxo-4H-1,2,4-oxadiazol- 3-yl)cyclopropyl]indole- 2-carbonyl]-2-(4-fluoro- 3,5-dimethyl-phenyl)- 4-methyl-4,6,7,8- tetrahydropyrazolo [4,3-c]azepin-3-yl]-N-(1- methylisoquinolin-2-ium- 6-yl)cyclopropanecarboxamide formate | 891.3 |
| 299 Isomers A and B | 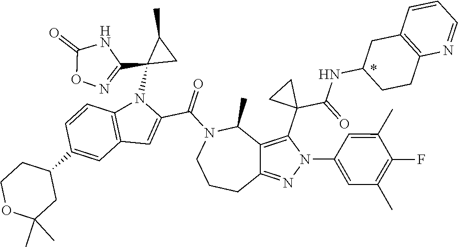 [SMILES: Cc1cc(-n2nc3c(c2C2(C(=O)NC4CCc5ncccc5C4)CC2)[C@H](C)N(C(=O)c2cc4cc([C@H]5CCOC(C)(C)C5)ccc4n2[C@@]2(c4noc(=O)[nH]4)C[C@@H]2C)CCC3)cc(C)c1F] | 1-((S)-5-(5-((S)-2,2- dimethyltetrahydro-2H- pyran-4-yl)-1-((1S,2S)-2- methyl-1-(5-oxo-4,5- dihydro-1,2,4-oxadiazol-3- yl)cyclopropyl)-1H-indole- 2-carbonyl)-2-(4-fluoro-3,5- dimethylphenyl)-4-methyl- 2,4,5,6,7,8-hexahydropyrazolo [4,3-c]azepin-3-yl)-N-((R)- 5,6,7,8-tetrahydroquinolin-6- yl)cyclopropane-1- carboxamide and 1-((S)-5- (5-((S)-2,2-dimethyltetrahydro- 2H-pyran-4-yl)-1-((1S,2S)- | 881.4 |
| | | 2-methyl-1-(5-oxo-4,5-dihydro- | |
| | | 1,2,4-oxadiazol-3-yl)cyclo- | |
| | | propyl)-1H-indole-2-carbonyl)- | |
| | | 2-(4-fluoro-3,5-dimethyl- | |
| | | phenyl)-4-methyl-2,4,5,6,7,8- | |
| | | hexahydropyrazolo[4,3-c] | |
| | | azepin-3-yl)-N-((S)-5,6,7,8- | |
| | | tetrahydroquinolin-6- | |
| | | yl)cyclopropane-1-carboxamide | |
| 300 Isomer A | 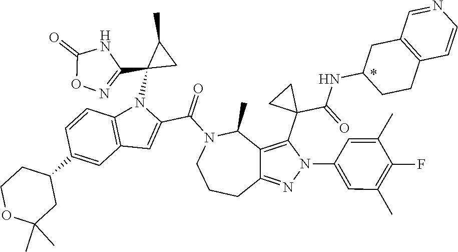 [SMILES: Cc1cc(-n2nc3c(c2C2(C(=O)NC4CCc5ccncc5C4)CC2)[C@H](C)N(C(=O)c2cc4cc([C@H]5CCOC(C)(C)C5)ccc4n2[C@@]2(c4noc(=O)[nH]4)C[C@@H]2C)CCC3)cc(C)c1F] | 1-((S)-5-(5-((S)-2,2- dimethyltetrahydro-2H- pyran-4-yl)-1-((1S,2S)-2- methyl-1-(5-oxo-4,5-dihydro- 1,2,4-oxadiazol-3-yl)cyclo- propyl)-1H-indole-2- carbonyl)-2-(4-fluoro-3,5- dimethylphenyl)-4-methyl- 2,4,5,6,7,8-hexahydropyrazolo [4,3-c]azepin-3-yl)-N-((R)- 5,6,7,8-tetrahydroisoquinolin- 7-yl)cyclopropane-1- carboxamide or 1-((S)-5-(5- ((S)-2,2-dimethyltetrahydro- 2H-pyran-4-yl)-1-((1S,2S)- 2-methyl-1-(5-oxo-4,5- | 881.4 |
| | | dihydro-1,2,4-oxadiazol-3- | |
| | | yl)cyclopropyl)-1H-indole- | |
| | | 2-carbonyl)-2-(4-fluoro-3,5- | |
| | | dimethylphenyl)-4-methyl- | |
| | | 2,4,5,6,7,8-hexahydropyrazolo | |
| | | [4,3-c]azepin-3-yl)-N-((S)- | |
| | | 5,6,7,8-tetrahydroisoquinolin-7- | |
| | | yl)cyclopropane-1-carboxamide | |
| 300 Isomer B | 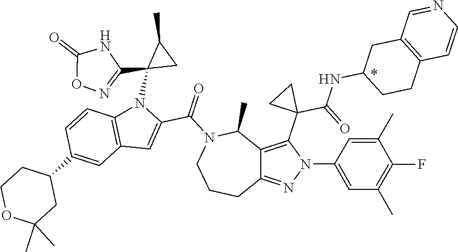 [SMILES: Cc1cc(-n2nc3c(c2C2(C(=O)NC4CCc5ccncc5C4)CC2)[C@H](C)N(C(=O)c2cc4cc([C@H]5CCOC(C)(C)C5)ccc4n2[C@@]2(c4noc(=O)[nH]4)C[C@@H]2C)CCC3)cc(C)c1F] | 1-((S)-5-(5-((S)-2,2- dimethyltetrahydro-2H- pyran-4-yl)-1-((1S,2S)-2- methyl-1-(5-oxo-4,5-dihydro- 1,2,4-oxadiazol-3-yl)cyclo- propyl)-1H-indole-2- carbonyl)-2-(4-fluoro-3,5- dimethylphenyl)-4-methyl- 2,4,5,6,7,8-hexahydropyrazolo [4,3-c]azepin-3-yl)-N-((R)- 5,6,7,8-tetrahydroisoquinolin- 7-yl)cyclopropane-1- carboxamide or 1-((S)-5- (5-((S)-2,2-dimethyltetrahydro- 2H-pyran-4-yl)-1-((1S,2S)- 2-methyl-1-(5-oxo-4,5- | 881.4 |
| | | dihydro-1,2,4-oxadiazol-3- | |
| | | yl)cyclopropyl)-1H-indole-2- | |
| | | carbonyl)-2-(4-fluoro-3,5- | |
| | | dimethylphenyl)-4-methyl- | |
| | | 2,4,5,6,7,8-hexahydro- | |
| | | pyrazolo[4,3-c]azepin- | |
| | | 3-yl)-N-((S)-5,6,7,8- | |
| | | tetrahydroisoquinolin-7- | |
| | | yl)cyclopropane-1- | |
| | | carboxamide | |
| 335 | 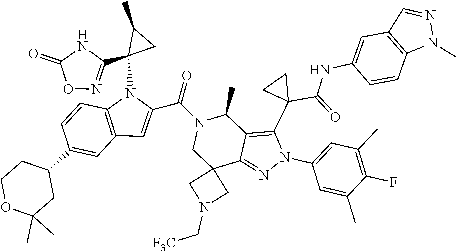 [SMILES: Cc1cc(-n2nc3c(c2C2(C(=O)Nc4ccc5c(cnn5C)c4)CC2)[C@H](C)N(C(=O)c2cc4cc([C@H]5CCOC(C)(C)C5)ccc4n2[C@@]2(c4noc(=O)[nH]4)C[C@@H]2C)CC32CN(CC(F)(F)F)C2)cc(C)c1F] | 1-((S)-5′-(5-((S)-2,2- dimethyltetrahydro-2H- pyran-4-yl)-1-((1S,2S)-2- methyl-1-(5-oxo-4,5-dihydro- 1,2,4-oxadiazol-3-yl)cyclo- propyl)-1H-indole-2- carbonyl)-2′-(4-fluoro-3,5- dimethylphenyl)-4′-methyl- 1-(2,2,2-trifluoroethyl)- 2′,4′,5′,6′-tetrahydrospiro [azetidine-3,7′-pyrazolo [4,3-c]pyridin]-3′-yl)-N- (4-fluoro-1-methyl-1H- indazol-5-yl)cyclopropane- 1-carboxamide | 1007.4 |
| 339 | 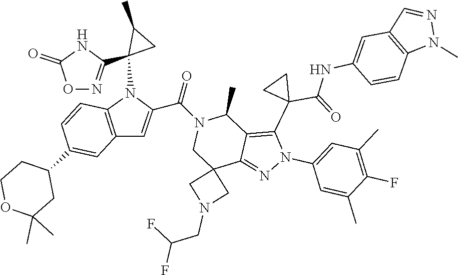 [SMILES: Cc1cc(-n2nc3c(c2C2(C(=O)Nc4ccc5c(cnn5C)c4)CC2)[C@H](C)N(C(=O)c2cc4cc([C@H]5CCOC(C)(C)C5)ccc4n2[C@@]2(c4noc(=O)[nH]4)C[C@@H]2C)CC32CN(CC(F)F)C2)cc(C)c1F] | 1-((S)-1-(2,2-difluoro- ethyl)-5′-(5-((S)-2,2- dimethyltetrahydro-2H- pyran-4-yl)-1-((1S,2S)-2- methyl-1-(5-oxo-4,5- dihydro-1,2,4-oxadiazol-3- yl)cyclopropyl)-1H-indole-2- carbonyl)-2′-(4-fluoro-3,5- dimethylphenyl)-4′-methyl- 2′,4′,5′,6′-tetrahydrospiro [azetidine-3,7′-pyrazolo [4,3-c]pyridin]-3′-yl)-N- (4-fluoro-1-methyl-1H- indazol-5-yl)cyclopropane- 1-carboxamide | 989.4 |
| 351 | 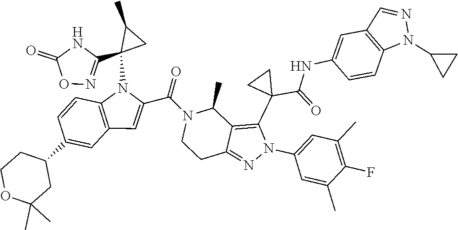 [SMILES: Cc1cc(-n2nc3c(c2C2(C(=O)Nc4ccc5c(cnn5C5CC5)c4)CC2)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | N-(1-cyclopropyl-1H- indazol-5-yl)-1-((S)-5-(5- ((S)-2,2-dimethyltetrahydro- 2H-pyran-4-yl)-1-((1S,2S)- 2-methyl-1-(5-oxo-4,5- dihydro-1,2,4-oxadiazol- 3-yl)cyclopropyl)-1H- indole-2-carbonyl)-2-(4- fluoro-3,5-dimethylphenyl)- 4-methyl-4,5,6,7-tetrahydro- 2H-pyrazolo[4,3-c]pyridin-3- yl)cyclopropane-1-carboxamide | 892.8 |
| 352 | 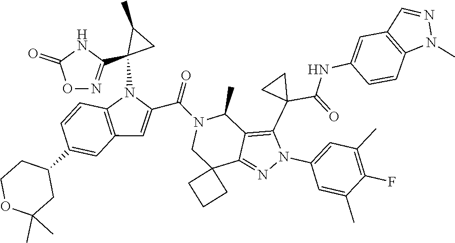 [SMILES: Cc1cc(-n2nc3c(c2C2(C(=O)Nc4ccc5c(cnn5C)c4)CC2)[C@H](C)N(C(=O)c2cc4cc([C@H]5CCOC(C)(C)C5)ccc4n2[C@@]2(c4noc(=O)[nH]4)C[C@@H]2C)CC32CCC2)cc(C)c1F] | 1-((S)-5′-(5-((S)-2,2- dimethyltetrahydro-2H- pyran-4-yl)-1-((1S,2S)-2- methyl-1-(5-oxo-4,5- dihydro-1,2,4-oxadiazol- 3-yl)cyclopropyl)-1H- indole-2-carbonyl)-2′- (4-fluoro-3,5-dimethyl- phenyl)-4′-methyl- 2′,4′,5′,6′-tetrahydrospiro [cyclobutane-1,7′- pyrazolo[4,3-c]pyridin]- 3′-yl)-N-(1-methyl-1H- indazol-5-yl)cyclopropane- 1-carboxamide | 896.7 |
| 385 | 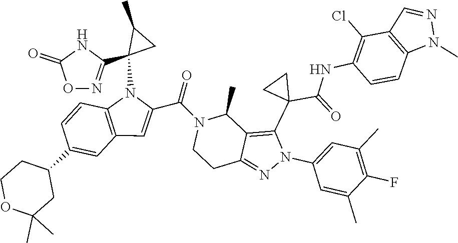 [SMILES: Cc1cc(-n2nc3c(c2C2(C(=O)Nc4ccc5c(cnn5C)c4Cl)CC2)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | N-(4-chloro-1-methyl- 1H-indazol-5-yl)-1-((S)- 5-(5-((S)-2,2-dimethyl- tetrahydro-2H-pyran-4- yl)-1-((1S,2S)-2-methyl- 1-(5-oxo-4,5-dihydro- 1,2,4-oxadiazol-3-yl) cyclopropyl)-1H-indole- 2-carbonyl)-2-(4-fluoro- 3,5-dimethylphenyl)-4- methyl-4,5,6,7-tetrahydro- 2H-pyrazolo[4,3-c]pyridin- 3-yl)cyclopropane-1- carboxamide | 900.2 |
| 386 | 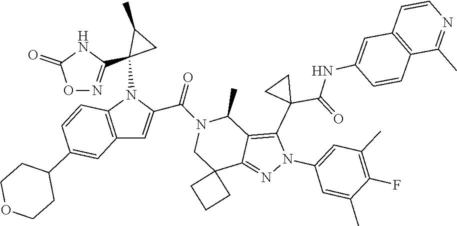 [SMILES: Cc1cc(-n2nc3c(c2C2(C(=O)Nc4ccc5c(C)nccc5c4)CC2)[C@H](C)N(C(=O)c2cc4cc(C5CCOCC5)ccc4n2[C@@]2(c4noc(=O)[nH]4)C[C@@H]2C)CC32CCC2)cc(C)c1F] | 1-((S)-2′-(4-fluoro-3,5- dimethylphenyl)-4′- methyl-5′-(1-((1S,2S)-2- methyl-1-(5-oxo-4,5- dihydro-1,2,4-oxadiazol- 3-yl)cyclopropyl)-5- (tetrahydro-2H-pyran- 4-yl)-1H-indole-2- carbonyl)-2′,4′,5′,6′- tetrahydrospiro[cyclo- butane-1,7′-pyrazolo [4,3-c]pyridin]-3′-yl)-N- (1-methylisoquinolin-6- yl)cyclopropane-1- | 889.7 |
| | | carboxamide | |
| 387 | 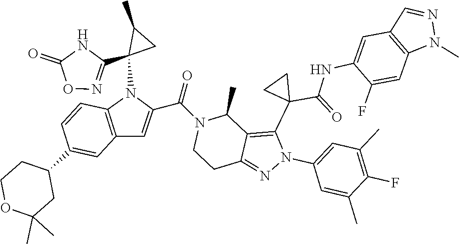 [SMILES: Cc1cc(-n2nc3c(c2C2(C(=O)Nc4cc5cnn(C)c5cc4F)CC2)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | 1-((S)-5-(5-((S)-2,2- dimethyltetrahydro-2H- pyran-4-yl)-1-((1S,2S)- 2-methyl-1-(5-oxo-4,5- dihydro-1,2,4-oxadiazol- 3-yl)cyclopropyl)-1H- indole-2-carbonyl)-2-(4- fluoro-3,5-dimethylphenyl)- 4-methyl-4,5,6,7-tetrahydro- 2H-pyrazolo[4,3-c]pyridin- 3-yl)-N-(6-fluoro-1- methyl-1H-indazol-5- yl)cyclopropane-1- carboxamide | 884.2 |
| 388 | 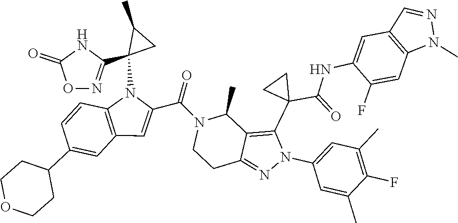 [SMILES: Cc1cc(-n2nc3c(c2C2(C(=O)Nc4cc5cnn(C)c5cc4F)CC2)[C@H](C)N(C(=O)c2cc4cc(C5CCOCC5)ccc4n2[C@@]2(c4noc(=O)[nH]4)C[C@@H]2C)CC3)cc(C)c1F] | N-(6-fluoro-1-methyl- 1H-indazol-5-yl)-1-((S)- 2-(4-fluoro-3,5-dimethyl- phenyl)-4-methyl-5-(1- ((1S,2S)-2-methyl-1-(5- oxo-4,5-dihydro-1,2,4- oxadiazol-3-yl)cyclopropyl)- 5-(tetrahydro-2H-pyran-4- yl)-1H-indole-2-carbonyl)- 4,5,6,7-tetrahydro-2H- pyrazolo[4,3-c]pyridin-3- yl)cyclopropane-1- carboxamide | 856.7 |
| 389 Isomer A | 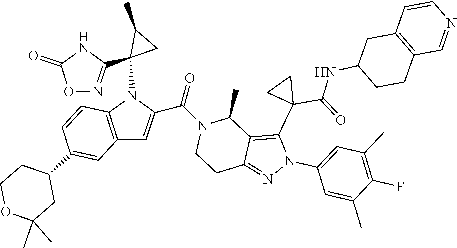 [SMILES: Cc1cc(-n2nc3c(c2C2(C(=O)NC4CCc5cnccc5C4)CC2)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | 1-((S)-5-(5-((S)-2,2- dimethyltetrahydro-2H- pyran-4-yl)-1-((1S,2S)- 2-methyl-1-(5-oxo-4,5- dihydro-1,2,4-oxadiazol- 3-yl)cyclopropyl)-1H- indole-2-carbonyl)-2- (4-fluoro-3,5-dimethyl- phenyl)-4-methyl-4,5,6,7- tetrahydro-2H-pyrazolo [4,3-c]pyridin-3-yl)-N- ((R)-5,6,7,8-tetrahydro- isoquinolin-6-yl)cyclo- propane-1-carboxamide OR | 867.7 |
| | | 1-((S)-5-(5-((S)-2,2- | |
| | | dimethyltetrahydro-2H- | |
| | | pyran-4-yl)-1-((1S,2S)- | |
| | | 2-methyl-1-(5-oxo- | |
| | | 4,5-dihydro-1,2,4-oxa- | |
| | | diazol-3-yl)cyclopropyl)- | |
| | | 1H-indole-2-carbonyl)- | |
| | | 2-(4-fluoro-3,5-dimethyl- | |
| | | phenyl)-4-methyl-4,5,6,7- | |
| | | tetrahydro-2H-pyrazolo | |
| | | [4,3-c]pyridin-3-yl)-N- | |
| | | ((S)-5,6,7,8-tetrahydro- | |
| | | isoquinolin-6-yl)cyclopro- | |
| | | pane-1-carboxamide | |
| 389 Isomer B | 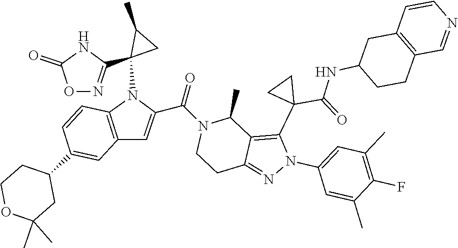 [SMILES: Cc1cc(-n2nc3c(c2C2(C(=O)NC4CCc5cnccc5C4)CC2)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | 1-((S)-5-(5-((S)-2,2- dimethyltetrahydro-2H- pyran-4-yl)-1-((1S,2S)-2- methyl-1-(5-oxo-4,5- dihydro-1,2,4-oxadiazol- 3-yl)cyclopropyl)-1H- indole-2-carbonyl)-2- (4-fluoro-3,5-dimethyl- phenyl)-4-methyl-4,5,6,7- tetrahydro-2H-pyrazolo [4,3-c]pyridin-3-yl)-N- ((R)-5,6,7,8-tetrahydro- isoquinolin-6-yl)cyclo- propane-1-carboxamide OR | 867.7 |
| | | 1-((S)-5-(5-((S)-2,2- | |
| | | dimethyltetrahydro-2H- | |
| | | pyran-4-yl)-1-((1S,2S)- | |
| | | 2-methyl-1-(5-oxo-4,5- | |
| | | dihydro-1,2,4-oxadiazol- | |
| | | 3-yl)cyclopropyl)-1H- | |
| | | indole-2-carbonyl)-2-(4- | |
| | | fluoro-3,5-dimethyl- | |
| | | phenyl)-4-methyl-4,5,6,7- | |
| | | tetrahydro-2H-pyrazolo | |
| | | [4,3-c]pyridin-3-yl)-N- | |
| | | ((S)-5,6,7,8-tetrahydro- | |
| | | isoquinolin-6-yl)cyclopro- | |
| | | pane-1-carboxamide | |
| 390 | 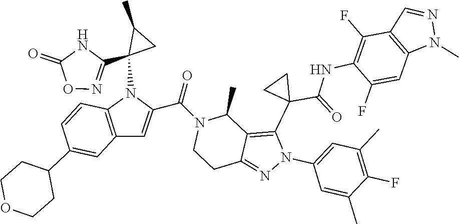 [SMILES: Cc1cc(-n2nc3c(c2C2(C(=O)Nc4c(F)cc5c(cnn5C)c4F)CC2)[C@H](C)N(C(=O)c2cc4cc(C5CCOCC5)ccc4n2[C@@]2(c4noc(=O)[nH]4)C[C@@H]2C)CC3)cc(C)c1F] | N-(4,6-difluoro-1-methyl- 1H-indazol-5-yl)-1-((S)- 2-(4-fluoro-3,5-dimethyl- phenyl)-4-methyl-5-(1- ((1S,2S)-2-methyl-1-(5- oxo-4,5-dihydro-1,2,4- oxadiazol-3-yl)cyclopropyl)- 5-(tetrahydro-2H-pyran-4- yl)-1H-indole-2-carbonyl)- 4,5,6,7-tetrahydro-2H- pyrazolo[4,3-c]pyridin-3- yl)cyclopropane-1- carboxamide | 874.6 |
| 391 | 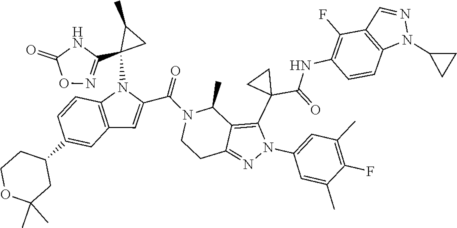 [SMILES: Cc1cc(-n2nc3c(c2C2(C(=O)Nc4ccc5c(cnn5C5CC5)c4F)CC2)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | N-(1-cyclopropyl-4- fluoro-1H-indazol-5- yl)-1-((S)-5-(5-((S)- 2,2-dimethyltetrahydro- 2H-pyran-4-yl)-1-((1S,2S)- 2-methyl-1-(5-oxo-4,5- dihydro-1,2,4-oxadiazol-3- yl)cyclopropyl)-1H-indole- 2-carbonyl)-2-(4-fluoro-3,5- dimethylphenyl)-4-methyl- 4,5,6,7-tetrahydro-2H- pyrazolo[4,3-c]pyridin-3-yl) cyclopropane-1-carboxamide | 910.8 |
| 392 | 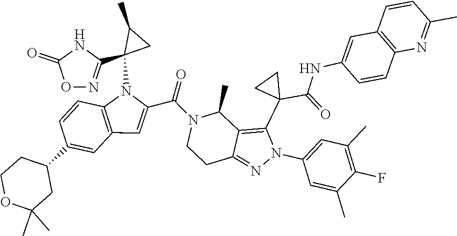 [SMILES: Cc1ccc2cc(NC(=O)C3(c4c5c(nn4-c4cc(C)c(F)c(C)c4)CCN(C(=O)c4cc6cc([C@H]7CCOC(C)(C)C7)ccc6n4[C@@]4(c6noc(=O)[nH]6)C[C@@H]4C)[C@H]5C)CC3)ccc2n1] | 1-((S)-5-(5-((S)-2,2- dimethyltetrahydro-2H- pyran-4-yl)-1-((1S,2S)-2- methyl-1-(5-oxo-4,5-dihydro- 1,2,4-oxadiazol-3-yl)cyclo- propyl)-1H-indole-2- carbonyl)-2-(4-fluoro-3,5- dimethylphenyl)-4-methyl- 4,5,6,7-tetrahydro-2H- pyrazolo[4,3-c]pyridin-3- yl)-N-(2-methylquinolin- 6-yl)cyclopropane-1- carboxamide | 877.7 |
| 393 | 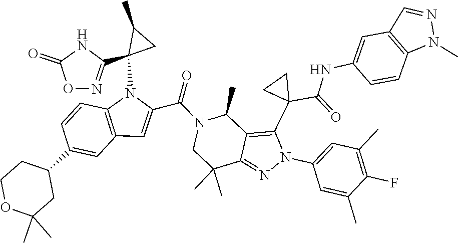 [SMILES: Cc1cc(-n2nc3c(c2C2(C(=O)Nc4ccc5c(cnn5C)c4)CC2)[C@H](C)N(C(=O)c2cc4cc([C@H]5CCOC(C)(C)C5)ccc4n2[C@@]2(c4noc(=O)[nH]4)C[C@@H]2C)CC3(C)C)cc(C)c1F] | 1-((S)-5-(5-((S)-2,2- dimethyltetrahydro-2H- pyran-4-yl)-1-((1S,2S)-2- methyl-1-(5-oxo-4,5- dihydro-1,2,4-oxadiazol- 3-yl)cyclopropyl)-1H-indole- 2-carbonyl)-2-(4-fluoro-3,5- dimethylphenyl)-4,7,7- trimethyl-4,5,6,7- tetrahydro-2H-pyrazolo [4,3-c]pyridin-3-yl)-N-(1- methyl-1H-indazol-5- yl)cyclopropane-1- carboxamide | 884.8 |
| 394 | 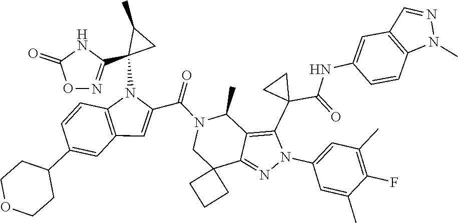 [SMILES: Cc1cc(-n2nc3c(c2C2(C(=O)Nc4ccc5c(cnn5C)c4)CC2)[C@H](C)N(C(=O)c2cc4cc(C5CCOCC5)ccc4n2[C@@]2(c4noc(=O)[nH]4)C[C@@H]2C)CC32CCC2)cc(C)c1F] | 1-((S)-2′-(4-fluoro-3,5- dimethylphenyl)-4′- methyl-5′-(1-((1S,2S)-2- methyl-1-(5-oxo-4,5- dihydro-1,2,4-oxadiazol- 3-yl)cyclopropyl)-5- (tetrahydro-2H-pyran-4- yl)-1H-indole-2- carbonyl)-2′,4′,5′,6′- tetrahydrospiro[cyclo- butane-1,7′-pyrazolo[4,3- c]pyridin]-3′-yl)-N- (1-methyl-1H-indazol-5- yl)cyclopropane-1- | 896.7 |
| | | carboxamide | |
| 395 | 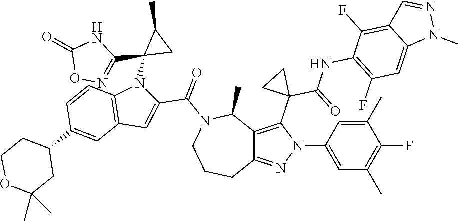 [SMILES: Cc1cc(-n2nc3c(c2C2(C(=O)Nc4c(F)cc5c(cnn5C)c4F)CC2)[C@H](C)N(C(=O)c2cc4cc([C@H]5CCOC(C)(C)C5)ccc4n2[C@@]2(c4noc(=O)[nH]4)C[C@@H]2C)CCC3)cc(C)c1F] | N-(4,6-difluoro-1- methyl-1H-indazol- 5-yl)-1-((S)-5-(5-((S)- 2,2-dimethyltetrahydro- 2H-pyran-4-yl)-1- ((1S,2S)-2-methyl-1- (5-oxo-4,5-dihydro- 1,2,4-oxadiazol-3- yl)cyclopropyl)-1H- indole-2-carbonyl)-2- (4-fluoro-3,5-dimethyl- phenyl)-4-methyl- 2,4,5,6,7,8-hexahydro- pyrazolo[4,3-c]azepin- | 916.7 |
| | | 3-yl)cyclopropane-1- | |
| | | carboxamide | |
| 396 | 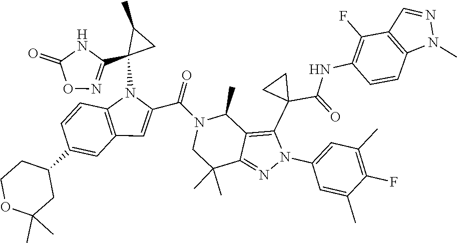 [SMILES: Cc1cc(-n2nc3c(c2C2(C(=O)Nc4ccc5c(cnn5C)c4F)CC2)[C@H](C)N(C(=O)c2cc4cc([C@H]5CCOC(C)(C)C5)ccc4n2[C@@]2(c4noc(=O)[nH]4)C[C@@H]2C)CC3(C)C)cc(C)c1F] | 1-((S)-5-(5-((S)-2,2- dimethyltetrahydro-2H- pyran-4-yl)-1-((1S,2S)- 2-methyl-1-(5-oxo-4,5- dihydro-1,2,4-oxadiazol- 3-yl)cyclopropyl)-1H- indole-2-carbonyl)-2-(4- fluoro-3,5-dimethylphenyl)- 4,7,7-trimethyl-4,5,6,7- tetrahydro-2H-pyrazolo [4,3-c]pyridin-3-yl)-N-(4- fluoro-1-methyl-1H-indazol- 5-yl)cyclopropane-1- carboxamide | 912.1 |
| 397 | 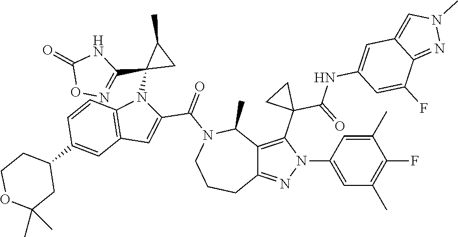 [SMILES: Cc1cc(-n2nc3c(c2C2(C(=O)Nc4cc(F)c5nn(C)cc5c4)CC2)[C@H](C)N(C(=O)c2cc4cc([C@H]5CCOC(C)(C)C5)ccc4n2[C@@]2(c4noc(=O)[nH]4)C[C@@H]2C)CCC3)cc(C)c1F] | 1-((S)-5-(5-((S)-2,2- dimethyltetrahydro-2H- pyran-4-yl)-1-((1S,2S)-2- methyl-1-(5-oxo-4,5-dihydro- 1,2,4-oxadiazol-3-yl)cyclo- propyl)-1H-indole-2-carbonyl)- 2-(4-fluoro-3,5-dimethyl- phenyl)-4-methyl-2,4,5,6,7,8- hexahydropyrazolo[4,3-c] azepin-3-yl)-N-(7-fluoro- 2-methyl-2H-indazol-5-yl) cyclopropane-1- carboxamide | 898.8 |
| 398 | 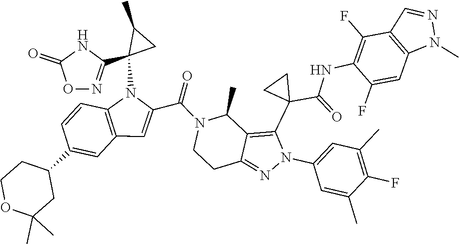 [SMILES: Cc1cc(-n2nc3c(c2C2(C(=O)Nc4c(F)cc5c(cnn5C)c4F)CC2)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | N-(4,6-difluoro-1 -methyl-1H-indazol-5- yl)-1-((S)-5-(5-((S)-2,2- dimethyltetrahydro-2H- pyran-4-yl)-1-((1S,2S)-2- methyl-1-(5-oxo-4,5- dihydro-1,2,4-oxadiazol- 3-yl)cyclopropyl)-1H- indole-2-carbonyl)-2-(4- fluoro-3,5-dimethylphenyl)- 4-methyl-4,5,6,7-tetrahydro- 2H-pyrazolo[4,3-c]pyridin- 3-yl)cyclopropane-1- carboxamide | 902.7 |
| 399 | 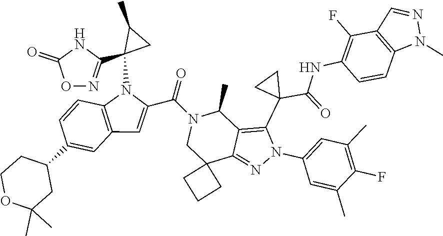 [SMILES: Cc1cc(-n2nc3c(c2C2(C(=O)Nc4ccc5c(cnn5C)c4F)CC2)[C@H](C)N(C(=O)c2cc4cc([C@H]5CCOC(C)(C)C5)ccc4n2[C@@]2(c4noc(=O)[nH]4)C[C@@H]2C)CC32CCC2)cc(C)c1F] | 1-((S)-5′-(5-((S)-2,2- dimethyltetrahydro-2H- pyran-4-yl)-1-((1S,2S)-2- methyl-1-(5-oxo-4,5- dihydro-1,2,4-oxadiazol- 3-yl)cyclopropyl)-1H- indole-2-carbonyl)-2′- (4-fluoro-3,5-dimethyl- phenyl)-4′-methyl- 2′,4′,5′,6′-tetrahydrospiro [cyclobutane-1,7′-pyrazolo [4,3-c]pyridin]-3′-yl)-N- (4-fluoro-1-methyl-1H- indazol-5-yl)cyclopropane- 1-carboxamide | 924.7 |
| 401 | 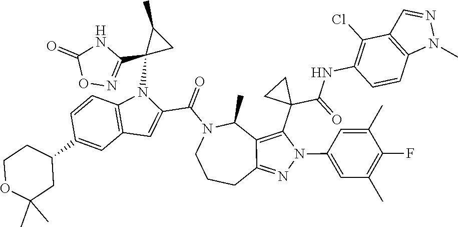 [SMILES: Cc1cc(-n2nc3c(c2C2(C(=O)Nc4ccc5c(cnn5C)c4Cl)CC2)[C@H](C)N(C(=O)c2cc4cc([C@H]5CCOC(C)(C)C5)ccc4n2[C@@]2(c4noc(=O)[nH]4)C[C@@H]2C)CCC3)cc(C)c1F] | N-(4-chloro-1-methyl- 1H-indazol-5-yl)-1- ((S)-5-(5-((S)-2,2-dimethyl- tetrahydro-2H-pyran-4- yl)-1-((1S,2S)-2-methyl- 1-(5-oxo-4,5-dihydro- 1,2,4-oxadiazol-3-yl) cyclopropyl)-1H-indole-2- carbonyl)-2-(4-fluoro-3,5- dimethylphenyl)-4-methyl- 2,4,5,6,7,8-hexahydro- pyrazolo[4,3-c]azepin- 3-yl)cyclopropane-1- carboxamide | 914.2 |
| 402 | 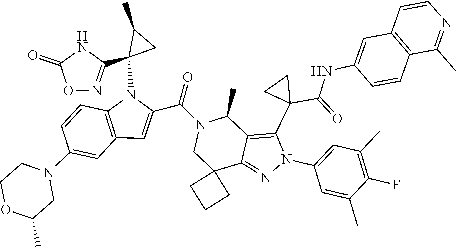 [SMILES: Cc1cc(-n2nc3c(c2C2(C(=O)Nc4ccc5c(C)nccc5c4)CC2)[C@H](C)N(C(=O)c2cc4cc(N5CCO[C@@H](C)C5)ccc4n2[C@@]2(c4noc(=O)[nH]4)C[C@@H]2C)CC32CCC2)cc(C)c1F] | 1-((S)-2′-(4-fluoro-3,5- dimethylphenyl)-4′- methyl-5′-(1-((1S,2S)-2- methyl-1-(5-oxo-4,5- dihydro-1,2,4-oxadiazol- 3-yl)cyclopropyl)-5-((S)- 2-methylmorpholino)- 1H-indole-2-carbonyl)- 2′,4′,5′,6′-tetrahydrospiro [cyclobutane-1,7′-pyrazolo [4,3-c]pyridin]-3′-yl)-N- (1-methylisoquinolin-6- yl)cyclopropane-1- carboxamide | 904.7 |

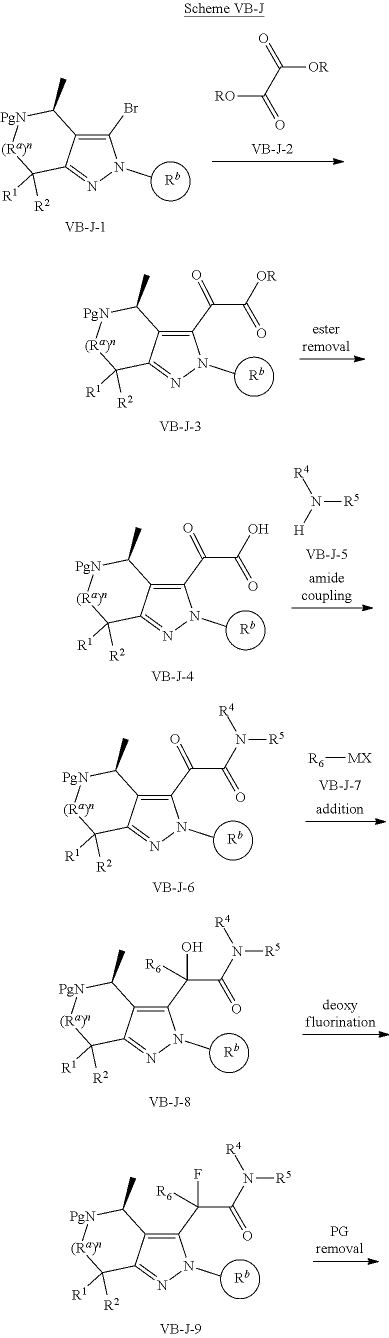

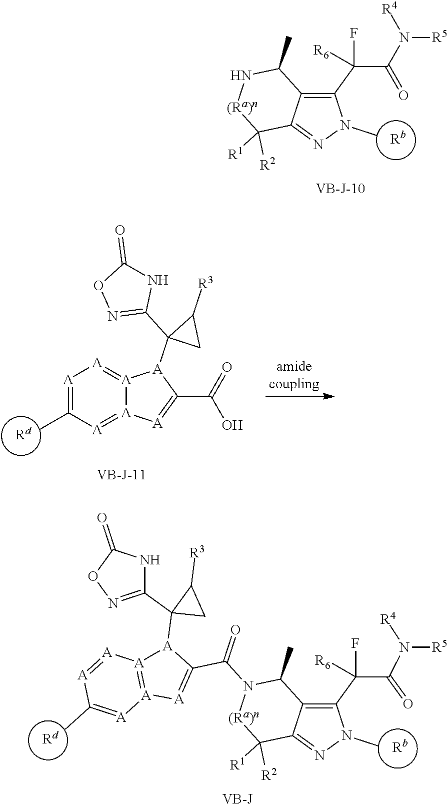

Compounds of formula (VB-J) are prepared from intermediate VB-J-1 by performing a metal-halogen exchange followed by trapping with dialkyl oxalate (VB-J-2). Removal of the ester (e.g., basic aqueous conditions) of VB-J-3 furnishes carboxylic acid VB-J-4. Subsequently, VB-J-4 is coupled with amines VB-J-5 to provide amide VB-J-6. Addition of organometallic reagent VB-J-7forms tertiary alcohol VB-J-8. Deoxyfluorination yields fluoroamide VB-J-9. Removal of the protecting group (e.g., acidic conditions) of VB-J-9provides amine VB-J-10 that is then coupled with carboxylic acid VB-J-11 to provide compounds of formula (VB-J).

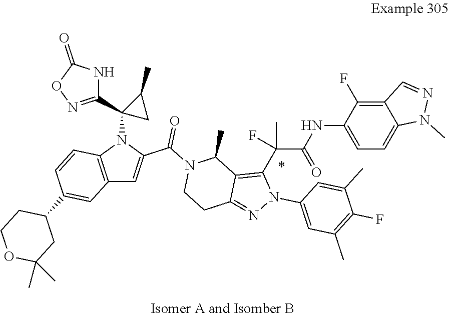 [SMILES: Cc1cc(-n2nc3c(c2C(C)(F)C(=O)Nc2ccc4c(cnn4C)c2F)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F]

### (R)-2-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)-2-fluoro-N-(4-fluoro-1-methyl-1H-indazol-5-yl)propenamide (305A) and (S)-2-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)-2-fluoro-N-(4-fluoro-1-methyl-1H-indazol-5-yl)propenamide (305B)

### Step 1: tert-butyl (S)-3-(2-ethoxy-2-oxoacetyl)-2-(4-fluoro-3,5-dimethylphenyl)-4-methyl-2,4,6,7-tetrahydro-5H-pyrazolo[4,3-c]pyridine-5-carboxylate

Tert-butyl (S)-3-bromo-2-(4-fluoro-3,5-dimethylphenyl)-4-methyl-2,4,6,7-tetrahydro-5H-pyrazolo[4,3-c]pyridine-5-carboxylate (1.47 g, 3.35 mmol) was added to a 40 mL vial equipped with a stir bar. The vial was capped, sparged with argon and THF (15 mL) was added. After cooling to −78° C., n-butyllithium in hexanes (322 mg, 3.12 mL, 1.6 molar, 5.02 mmol) was added dropwise. Five minutes later, diethyl oxalate (978 mg, 909 μL, 6.69 mmol) was added dropwise and the reaction was warmed to room temperature. The reaction was quenched with careful addition of sat. NH₄Cl solution and diluted with EtOAc. The mixture was extracted with EtOAc, and combined organic layers were washed with brine, dried with MgSO₄ and concentrated under reduced pressure. Purification was achieved by normal phase silica gel chromatography (40 g cartridge, 0-80% EtOAc/hexanes) to afford the product as an orange residue. MS (ESI) m/z: 460.2 [M+H]⁺.

### Step 2: (S)-2-(5-(tert-butoxycarbonyl)-2-(4-fluoro-3,5-dimethylphenyl)-4-methyl-4,5,6,7-tetrahydro-2H-pyrazolo[4,3-c]pyridin-3-yl)-2-oxoacetic acid

In a 20 mL vial, tert-butyl (S)-3-(2-ethoxy-2-oxoacetyl)-2-(4-fluoro-3,5-dimethylphenyl)-4-methyl-2,4,6,7-tetrahydro-5H-pyrazolo[4,3-c]pyridine-5-carboxylate (925 mg, 2.01 mmol) was dissolved in THF/MeOH=1:1 (1 mL). An aqueous solution of LiOH H₂O (422 mg, 10.1 mmol) in 0.5 mL of water was added to the organic solution. The mixture was stirred at 40° C. for 0.5 hours, at which time the mixture was acidified with 1 N HCl solution and diluted with EtOAc. The mixture was extracted with EtOAc and combined organic layers were washed with brine, dried with MgSO₄ and concentrated under reduced pressure. The crude was used in subsequent steps without further purification. MS (ESI) m/z: 432.2 [M+H]⁺.

### Step 3: tert-butyl (S)-3-(2-((4-fluoro-1-methyl-1H-indazol-5-yl)amino)-2-oxoacetyl)-2-(4-fluoro-3,5-dimethylphenyl)-4-methyl-2,4,6,7-tetrahydro-5H-pyrazolo[4,3-c]pyridine-5-carboxylate

In a 40 mL vial, (S)-2-(5-(tert-butoxycarbonyl)-2-(4-fluoro-3,5-dimethylphenyl)-4-methyl-4,5,6,7-tetrahydro-2H-pyrazolo[4,3-c]pyridin-3-yl)-2-oxoacetic acid (839 mg, 1.94 mmol), N-ethyl-N-isopropylpropan-2-amine (1.26 g, 9.72 mmol), HATU (887 mg, 2.33 mmol) were dissolved in DMF (1.5 mL) and 4-fluoro-1-methyl-1H-indazol-5-amine, HCl (470 mg, 2.33 mmol) was added. The mixture was stirred at room temperature for four hours, at which time LCMS analysis suggested consumption of starting material. The reaction concentrated and purified by normal phase silica gel chromatography using 0-100% EtOAc in hex over 15 min to afford the product. MS (ESI) m/z: 579.2 [M+H]⁺.

### Step 4: tert-butyl (4S)-3-(1-((4-fluoro-1-methyl-1H-indazol-5-yl)amino)-2-hydroxy-1-oxopropan-2-yl)-2-(4-fluoro-3,5-dimethylphenyl)-4-methyl-2,4,6,7-tetrahydro-5H-pyrazolo[4,3-c]pyridine-5-carboxylate

In a 20 mL vial, tert-butyl (S)-3-(2-((4-fluoro-1-methyl-1H-indazol-5-yl)amino)-2-oxoacetyl)-2-(4-fluoro-3,5-dimethylphenyl)-4-methyl-2,4,6,7-tetrahydro-5H-pyrazolo[4,3-c]pyridine-5-carboxylate (555 mg, 959 mol) was dissolved in THF (4.80 mL) and the solution was cooled to 0° C. Methylmagnesium bromide (355 mg, 3 molar, 2.97 mmol) was added dropwise. 30 minutes later, LCMS analysis suggested consumption of starting material and formation of product. The reaction was treated with aqueous NH₄Cl solution and extracted with EtOAc. Combined organic layers were washed with brine, dried with MgSO₄ and concentrated under reduced pressure. Purification was achieved by normal phase silica gel chromatography using 0-100% EtOAc in hexanes over 15 min to afford the product. MS (ESI) m/z: 595.2 [M+H]⁺.

### Step 5: tert-butyl (4S)-3-(2-fluoro-1-((4-fluoro-1-methyl-1H-indazol-5-yl)amino)-1-oxopropan-2-yl)-2-(4-fluoro-3,5-dimethylphenyl)-4-methyl-2,4,6,7-tetrahydro-5H-pyrazolo[4,3-c]pyridine-5-carboxylate

In a 20 mL dram vial, tert-butyl (4S)-3-(1-((4-fluoro-1-methyl-1H-indazol-5-yl)amino)-2-hydroxy-1-oxopropan-2-yl)-2-(4-fluoro-3,5-dimethylphenyl)-4-methyl-2,4,6,7-tetrahydro-5H-pyrazolo[4,3-c]pyridine-5-carboxylate (122 mg, 205 mol) was dissolved in DCM (2 mL). Deoxo-Fluor (99.9 mg, 87.5 μL, 50% wt, 226 μmol) was added dropwise at 0° C. The mixture was warmed to RT after addition. 10 min later, the reaction was quenched with saturated NaHCO₃ solution. The mixture was extracted with DCM and combined organic layers were dried with MgSO₄ and concentrated under reduced pressure. Purification was achieved by normal phase silica gel chromatography using 0-100% EtOAc in hexanes over 15 min to afford the product as a residue. MS (ESI) m/z: 597.4 [M+H]⁺.

### Step 6: 2-fluoro-N-(4-fluoro-1-methyl-1H-indazol-5-yl)-2-((S)-2-(4-fluoro-3,5-dimethylphenyl)-4-methyl-4,5,6,7-tetrahydro-2H-pyrazolo[4,3-c]pyridin-3-yl)propanamide, HCl

In a 20 mL vial, tert-butyl (4S)-3-(2-fluoro-1-((4-fluoro-1-methyl-1H-indazol-5-yl)amino)-1-oxopropan-2-yl)-2-(4-fluoro-3,5-dimethylphenyl)-4-methyl-2,4,6,7-tetrahydro-5H-pyrazolo[4,3-c]pyridine-5-carboxylate (149 mg, 250 mol) was treated with hydrogen chloride (91.2 mg, 626 μL, 4 molar in dioxane, 2.50 mmol) in dioxane at 25° C. The reaction mixture was stirred for 30 minutes at this temperature. The reaction was concentrated under reduced pressure and used directly in the next step. MS (ESI) m/z: 497.2 [M+H]⁺.

### Step 7: (R)-2-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)-2-fluoro-N-(4-fluoro-1-methyl-1H-indazol-5-yl)propenamide and (S)-2-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)-2-fluoro-N-(4-fluoro-1-methyl-1H-indazol-5-yl)propanamide

In a 4 mL vial, HATU (100 mg, 263.1 mol), 5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carboxylic acid (108.27 mg, 263.14 mol), 2-fluoro-N-(4-fluoro-1-methyl-1H-indazol-5-yl)-2-((S)-2-(4-fluoro-3,5-dimethylphenyl)-4-methyl-4,5,6,7-tetrahydro-2H-pyrazolo[4,3-c]pyridin-3-yl)propanamide, HCl (116.9 mg, 219.3 mol), diisopropylethylamine (340. mg, 453 μL, 2.63 mmol) were dissolved in DMF (0.4 mL) and the reaction was stirred at 25° C. for 1.5 hours. The reaction mixture was filtered (Nalgene®, PTFE 0.45) and purified by reverse phase prep-HPLC (70-95% MeCN/water+0.1% formic acid modifier) followed by prep-SFC (Berger MGII SFC, Daicel ChiralPak®IC, 250 (L)×30 (ID) mm, 85 mL/min, 30% EtOH CO₂ modifier) to afford the title compounds. 1H NMR (500 MHz, DMSO-d6) δ 11.8 (s, 1H), 10.0 (s, 1H), 8.16-8.08 (m, 1H), 7.52-6.92 (m, 7H), 5.99-5.97 (m, 1H), 4.32-4.31 (m, 1H), 4.09-4.05 (m, 6H), 3.72-3.70 (m, 3H), 3.12-2.80 (m, 3H), 2.22 (s, 6H), 1.94-1.51 (m, 11H), 1.27-1.18 (m, 9H) (faster eluting, peak 1), 11.8 (s, 1H), 10.1 (s, 1H), 8.16-8.10 (m, 1H), 7.52-6.93 (m, 7H), 5.93-5.92 (m, 1H), 4.32-4.31 (m, 1H), 4.09-4.05 (m, 3H), 3.72-3.70 (m, 3H), 3.12-2.77 (m, 3H), 2.22-2.20 (m, 6H), 1.90-1.51 (m, 11H), 1.27-1.16 (m, 9H) (slower eluting, peak 2). MS (ESI) m/z: 890.4 [M+H]⁺. The following examples in table Table 26 were prepared according to scheme VB-J using the procedures outlined in the synthesis of Example 305. For some examples, Step 5 of Example 305 was skipped.

TABLE 26

| | | | MS |
|---|---|---|---|
| Ex. | Structure | Name | (M + 1) |
| 306 Isomer A (Daicel Chiralpak ® Whelk O (R,R), 25 (L) × 3 (ID) cm, 50% MeOH | 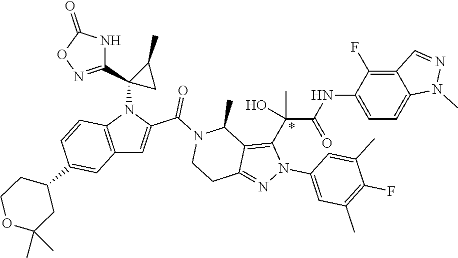 [SMILES: Cc1cc(-n2nc3c(c2C(C)(O)C(=O)Nc2ccc4c(cnn4C)c2F)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | (R)-2-((S)-5-(5-((S)-2,2- dimethyltetrahydro-2H-pyran-4-yl)-1- ((1S,2S)-2-methyl-1-(5-oxo-4,5-dihydro- 1,2,4-oxadiazol-3-yl)cyclopropyl)-1H- indole-2-carbonyl)-2-(4-fluoro-3,5- dimethylphenyl)-4-methyl-4,5,6,7- tetrahydro-2H-pyrazolo[4,3-c]pyridin-3- yl)-N-(4-fluoro-1-methyl-1H-indazol-5- yl)-2-hydroxypropanamide or (S)-2-((S)- 5-(5-((S)-2,2-dimethyltetrahydro-2H- pyran-4-yl)-1-((1S,2S)-2-methyl-1-(5- oxo-4,5-dihydro-1,2,4-oxadiazol-3- yl)cyclopropyl)-1H-indole-2-carbonyl)-2- (4-fluoro-3,5-dimethylphenyl)-4-methyl- 4,5,6,7-tetrahydro-2H-pyrazolo[4,3- c]pyridin-3-yl)-N-(4-fluoro-1-methyl-1H- | 888.4 |
| | | indazol-5-yl)-2-hydroxypropanamide | |
| 306 Isomer B | 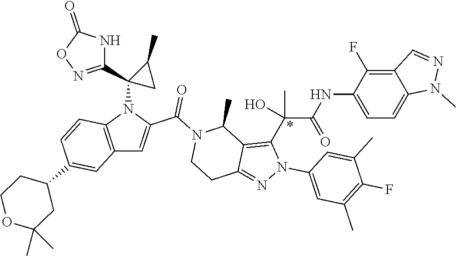 [SMILES: Cc1cc(-n2nc3c(c2C(C)(O)C(=O)Nc2ccc4c(cnn4C)c2F)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | (R)-2-((S)-5-(5-((S)-2,2- dimethyltetrahydro-2H-pyran-4-yl)-1- ((1S,2S)-2-methyl-1-(5-oxo-4,5-dihydro- 1,2,4-oxadiazol-3-yl)cyclopropyl)-1H- indole-2-carbonyl)-2-(4-fluoro-3,5- dimethylphenyl)-4-methyl-4,5,6,7- tetrahydro-2H-pyrazolo[4,3-c]pyridin-3- yl)-N-(4-fluoro-1-methyl-1H-indazol-5- yl)-2-hydroxypropanamide or (S)-2-((S)- 5-(5-((S)-2,2-dimethyltetrahydro-2H- pyran-4-yl)-1-((1S,2S)-2-methyl-1-(5- oxo-4,5-dihydro-1,2,4-oxadiazol-3- yl)cyclopropyl)-1H-indole-2-carbonyl)-2- (4-fluoro-3,5-dimethylphenyl)-4-methyl- 4,5,6,7-tetrahydro-2H-pyrazolo[4,3- c]pyridin-3-yl)-N-(4-fluoro-1-methyl-1H- | 888.4 |
| | | indazol-5-yl)-2-hydroxypropanamide | |
| 307 Isomer A (prep- HPLC 65- 95% MeCN: W2O + 0.1% TFA) | 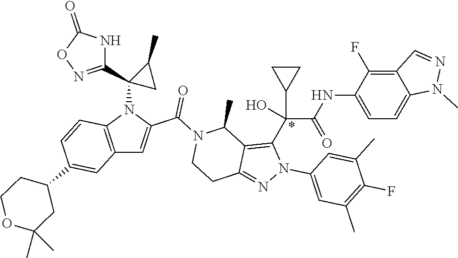 [SMILES: Cc1cc(-n2nc3c(c2C(O)(C(=O)Nc2ccc4c(cnn4C)c2F)C2CC2)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | (R)-2-cyclopropyl-2-((S)-5-(5-((S)-2,2- dimethyltetrahydro-2H-pyran-4-yl)-1- ((1S,2S)-2-methyl-1-(5-oxo-4,5-dihydro- 1,2,4-oxadiazol-3-yl)cyclopropyl)-1H- indole-2-carbonyl)-2-(4-fluoro-3,5- dimethylphenyl)-4-methyl-4,5,6,7- tetrahydro-2H-pyrazolo[4,3-c]pyridin-3- yl)-N-(4-fluoro-1-methyl-1H-indazol-5- yl)-2-hydroxyacetamide or (S)-2- cyclopropyl-2-((S)-5-(5-((S)-2,2- dimethyltetrahydro-2H-pyran-4-yl)-1- ((1S,2S)-2-methyl-1-(5-oxo-4,5-dihydro- 1,2,4-oxadiazol-3-yl)cyclopropyl)-1H- indole-2-carbonyl)-2-(4-fluoro-3,5- dimethylphenyl)-4-methyl-4,5,6,7- tetrahydro-2H-pyrazolo[4,3-c]pyridin-3- yl)-N-(4-fluoro-1-methyl-1H-indazol-5- yl)-2-hydroxyacetamide | 914.4 |
| 307 Isomer B (prep- HPLC 65- 95% MeCN: W2O + 0.1% TFA) | 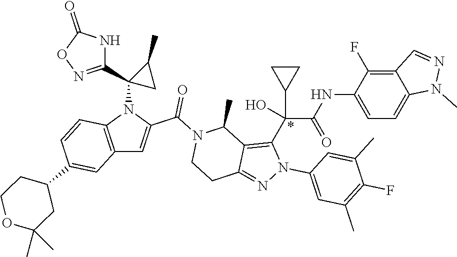 [SMILES: Cc1cc(-n2nc3c(c2C(O)(C(=O)Nc2ccc4c(cnn4C)c2F)C2CC2)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | (R)-2-cyclopropyl-2-((S)-5-(5-((S)-2,2- dimethyltetrahydro-2H-pyran-4-yl)-1- ((1S,2S)-2-methyl-1-(5-oxo-4,5-dihydro- 1,2,4-oxadiazol-3-yl)cyclopropyl)-1H- indole-2-carbonyl)-2-(4-fluoro-3,5- dimethylphenyl)-4-methyl-4,5,6,7- tetrahydro-2H-pyrazolo[4,3-c]pyridin-3- yl)-N-(4-fluoro-1-methyl-1H-indazol-5- yl)-2-hydroxyacetamide or (S)-2- cyclopropyl-2-((S)-5-(5-((S)-2,2- dimethyltetrahydro-2H-pyran-4-yl)-1- ((1S,2S)-2-methyl-1-(5-oxo-4,5-dihydro- 1,2,4-oxadiazol-3-yl)cyclopropyl)-1H- indole-2-carbonyl)-2-(4-fluoro-3,5- dimethylphenyl)-4-methyl-4,5,6,7- tetrahydro-2H-pyrazolo[4,3-c]pyridin-3- | 914.4 |
| | | yl)-N-(4-fluoro-1-methyl-1H-indazol-5- | |
| | | yl)-2-hydroxyacetamide | |
| 308 Isomer A (Whelk o-02, 30% MeOH in CO2 with 0.1% NH4OH) | 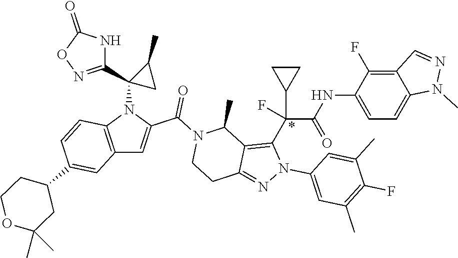 [SMILES: Cc1cc(-n2nc3c(c2C(F)(C(=O)Nc2ccc4c(cnn4C)c2F)C2CC2)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | (R)-2-cyclopropyl-2-((S)-5-(5-((S)-2,2- dimethyltetrahydro-2H-pyran-4-yl)-1- ((1S,2S)-2-methyl-1-(5-oxo-4,5-dihydro- 1,2,4-oxadiazol-3-yl)cyclopropyl)-1H- indole-2-carbonyl)-2-(4-fluoro-3,5- dimethylphenyl)-4-methyl-4,5,6,7- tetrahydro-2H-pyrazolo[4,3-c]pyridin-3- yl)-2-fluoro-N-(4-fluoro-1-methyl-1H- indazol-5-yl)acetamide or (S)-2- cyclopropyl-2-((S)-5-(5-((S)-2,2- dimethyltetrahydro-2H-pyran-4-yl)-1- ((1S,2S)-2-methyl-1-(5-oxo-4,5-dihydro- 1,2,4-oxadiazol-3-yl)cyclopropyl)-1H- indole-2-carbonyl)-2-(4-fluoro-3,5- dimethylphenyl)-4-methyl-4,5,6,7- tetrahydro-2H-pyrazolo[4,3-c]pyridin-3- | 916.4 |
| | | yl)-2-fluoro-N-(4-fluoro-1-methyl-1H- | |
| | | indazol-5-yl)acetamide | |
| 308 Isomer B (Whelk o-02, 30% MeOH in CO2 with 0.1% NH4OH) | 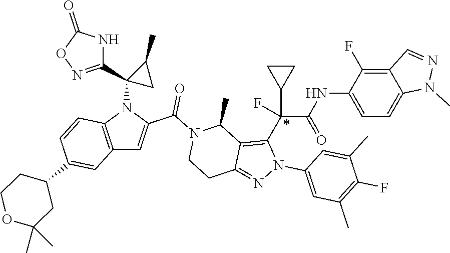 [SMILES: Cc1cc(-n2nc3c(c2C(F)(C(=O)Nc2ccc4c(cnn4C)c2F)C2CC2)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | (R)-2-cyclopropyl-2-((S)-5-(5-((S)-2,2- dimethyltetrahydro-2H-pyran-4-yl)-1- ((1S,2S)-2-methyl-1-(5-oxo-4,5-dihydro- 1,2,4-oxadiazol-3-yl)cyclopropyl)-1H- indole-2-carbonyl)-2-(4-fluoro-3,5- dimethylphenyl)-4-methyl-4,5,6,7- tetrahydro-2H-pyrazolo[4,3-c]pyridin-3- yl)-2-fluoro-N-(4-fluoro-1-methyl-1H- indazol-5-yl)acetamide or (S)-2- cyclopropyl-2-((S)-5-(5-((S)-2,2- dimethyltetrahydro-2H-pyran-4-yl)-1- ((1S,2S)-2-methyl-1-(5-oxo-4,5-dihydro- 1,2,4-oxadiazol-3-yl)cyclopropyl)-1H- indole-2-carbonyl)-2-(4-fluoro-3,5- dimethylphenyl)-4-methyl-4,5,6,7- tetrahydro-2H-pyrazolo[4,3-c]pyridin-3- | 916.4 |
| | | yl)-2-fluoro-N-(4-fluoro-1-methyl-1H- | |
| | | indazol-5-yl)acetamide | |
| 309 Isomer A (prep- HPLC 30- 45% MeCN: H2O + 0.1% FA) | 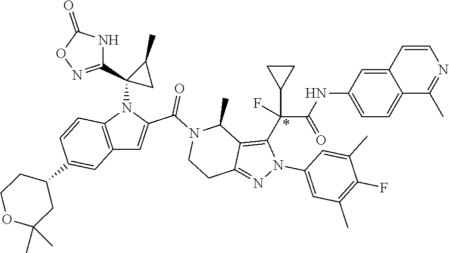 [SMILES: Cc1cc(-n2nc3c(c2C(F)(C(=O)Nc2ccc4c(C)nccc4c2)C2CC2)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | (R)-2-cyclopropyl-2-((S)-5-(5-((S)-2,2- dimethyltetrahydro-2H-pyran-4-yl)-1- ((1S,2S)-2-methyl-1-(5-oxo-4,5-dihydro- 1,2,4-oxadiazol-3-yl)cyclopropyl)-1H- indole-2-carbonyl)-2-(4-fluoro-3,5- dimethylphenyl)-4-methyl-4,5,6,7- tetrahydro-2H-pyrazolo[4,3-c]pyridin-3- yl)-2-fluoro-N-(1-methylisoquinolin-6- yl)acetamide or (S)-2-cyclopropyl-2-((S)- 5-(5-((S)-2,2-dimethyltetrahydro-2H- pyran-4-yl)-1-((1S,2S)-2-methyl-1-(5- oxo-4,5-dihydro-1,2,4-oxadiazol-3- yl)cyclopropyl)-1H-indole-2-carbonyl)-2- (4-fluoro-3,5-dimethylphenyl)-4-methyl- 4,5,6,7-tetrahydro-2H-pyrazolo[4,3- c]pyridin-3-yl)-2-fluoro-N-(1- | 909.4 |
| | | methylisoquinolin-6-yl)acetamide | |
| 309 Isomer B (prep- HPLC 30- 45% MeCN: H2O + 0.1% FA) | 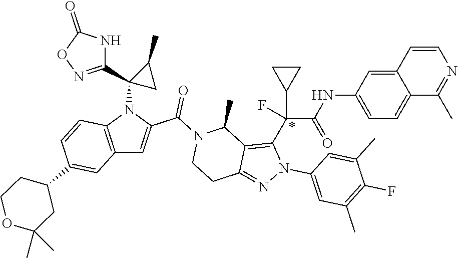 [SMILES: Cc1cc(-n2nc3c(c2C(F)(C(=O)Nc2ccc4c(C)nccc4c2)C2CC2)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | (R)-2-cyclopropyl-2-((S)-5-(5-((S)-2,2- dimethyltetrahydro-2H-pyran-4-yl)-1- ((1S,2S)-2-methyl-1-(5-oxo-4,5-dihydro- 1,2,4-oxadiazol-3-yl)cyclopropyl)-1H- indole-2-carbonyl)-2-(4-fluoro-3,5- dimethylphenyl)-4-methyl-4,5,6,7- tetrahydro-2H-pyrazolo[4,3-c]pyridin-3- yl)-2-fluoro-N-(1-methylisoquinolin-6- yl)acetamide or (S)-2-cyclopropyl-2-((S)- 5-(5-((S)-2,2-dimethyltetrahydro-2H- pyran-4-yl)-1-((1S,2S)-2-methyl-1-(5- oxo-4,5-dihydro-1,2,4-oxadiazol-3- yl)cyclopropyl)-1H-indole-2-carbonyl)-2- (4-fluoro-3,5-dimethylphenyl)-4-methyl- 4,5,6,7-tetrahydro-2H-pyrazolo[4,3- c]pyridin-3-yl)-2-fluoro-N-(1- | 909.4 |
| | | methylisoquinolin-6-yl)acetamide | |
| 403 | 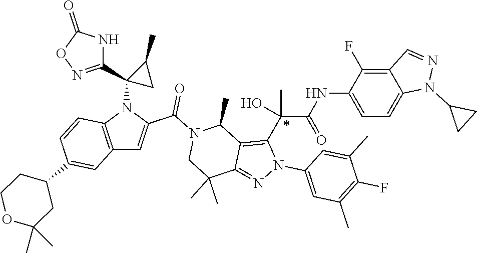 [SMILES: Cc1cc(-n2nc3c(c2C(C)(O)C(=O)Nc2ccc4c(cnn4C4CC4)c2F)[C@H](C)N(C(=O)c2cc4cc([C@H]5CCOC(C)(C)C5)ccc4n2[C@@]2(c4noc(=O)[nH]4)C[C@@H]2C)CC3(C)C)cc(C)c1F] | (S)-N-(1-cyclopropyl-4-fluoro-1H- indazol-5-yl)-2-((S)-5-(5-((S)-2,2- dimethyltetrahydro-2H-pyran-4-yl)-1- ((1S,2S)-2-methyl-1-(5-oxo-4,5-dihydro- 1,2,4-oxadiazol-3-yl)cyclopropyl)-1H- indole-2-carbonyl)-2-(4-fluoro-3,5- dimethylphenyl)-4,7,7-trimethyl-4,5,6,7- tetrahydro-2H-pyrazolo[4,3-c]pyridin-3- yl)-2-hydroxypropanamide OR (R)-N-(1- cyclopropyl-4-fluoro-1H-indazol-5-yl)-2- ((S)-5-(5-((S)-2,2-dimethyltetrahydro-2H- pyran-4-yl)-1-((1S,2S)-2-methyl-1-(5- oxo-4,5-dihydro-1,2,4-oxadiazol-3- yl)cyclopropyl)-1H-indole-2-carbonyl)-2- (4-fluoro-3,5-dimethylphenyl)-4,7,7- trimethyl-4,5,6,7-tetrahydro-2H- | 942.4 |
| | | pyrazolo[4,3-c]pyridin-3-yl)-2- | |
| | | hydroxypropanamide | |
| 404 | 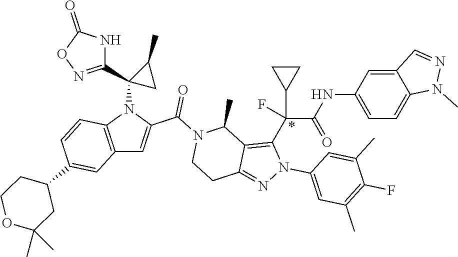 [SMILES: Cc1cc(-n2nc3c(c2C(F)(C(=O)Nc2ccc4c(cnn4C)c2)C2CC2)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F] | (S)-2-cyclopropyl-2-((S)-5-(5-((S)-2,2- dimethyltetrahydro-2H-pyran-4-yl)-1- ((1S,2S)-2-methyl-1-(5-oxo-4,5-dihydro- 1,2,4-oxadiazol-3-yl)cyclopropyl)-1H- indole-2-carbonyl)-2-(4-fluoro-3,5- dimethylphenyl)-4-methyl-4,5,6,7- tetrahydro-2H-pyrazolo[4,3-c]pyridin-3- yl)-2-fluoro-N-(1-methyl-1H-indazol-5- yl)acetamide OR (R)-2-cyclopropyl-2- ((S)-5-(5-((S)-2,2-dimethyltetrahydro-2H- pyran-4-yl)-1-((1S,2S)-2-methyl-1-(5- oxo-4,5-dihydro-1,2,4-oxadiazol-3- yl)cyclopropyl)-1H-indole-2-carbonyl)-2- (4-fluoro-3,5-dimethylphenyl)-4-methyl- 4,5,6,7-tetrahydro-2H-pyrazolo[4,3- c]pyridin-3-yl)-2-fluoro-N-(1-methyl-1H- indazol-5-yl)acetamide | 899.1 |

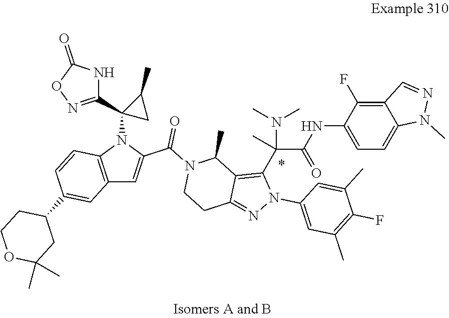 [SMILES: Cc1cc(-n2nc3c(c2C(C)(C(=O)Nc2ccc4c(cnn4C)c2F)N(C)C)[C@H](C)N(C(=O)c2cc4cc([C@H]5CCOC(C)(C)C5)ccc4n2[C@@]2(c4noc(=O)[nH]4)C[C@@H]2C)CC3)cc(C)c1F]

### (S)-2-(dimethylamino)-2-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)-N-(4-fluoro-1-methyl-1H-indazol-5-yl)propenamide or (R)-2-(dimethylamino)-2-((S)-5-(5-((S)-2,2-dimethyltetrahydro-2H-pyran-4-yl)-1-((1 S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)-N-(4-fluoro-1-methyl-1H-indazol-5-yl)propenamide

### Step 1: tert-butyl (4S)-3-(2-chloro-1-((4-fluoro-1-methyl-1H-indazol-5-yl)amino)-1-oxopropan-2-yl)-2-(4-fluoro-3,5-dimethylphenyl)-4-methyl-2,4,6,7-tetrahydro-5H-pyrazolo[4,3-c]pyridine-5-carboxylate

In a 1 dram vial, tert-butyl (4S)-3-(1-((4-fluoro-1-methyl-1H-indazol-5-yl)amino)-2-hydroxy-1-oxopropan-2-yl)-2-(4-fluoro-3,5-dimethylphenyl)-4-methyl-2,4,6,7-tetrahydro-5H-pyrazolo[4,3-c]pyridine-5-carboxylate (57 mg, 96 mol) was dissolved in DCM (1 mL) and diisopropylethylamine (25 mg, 0.19 mmol) was added. The solution was cooled to 0° C. and thionyl chloride (23 mg, 2 M in DCM, 0.19 mmol) was added dropwise. The reaction was stirred at 0° C. for 1.5 hours at which time the mixture was purified by silica gel column chromatography (0-100% EtOAc in hexanes) to provide the title compound. MS (ESI) m/z: 613.2 [M+H]+.

### Step 2: tert-butyl (4S)-3-(2-(dimethylamino)-1-((4-fluoro-1-methyl-1H-indazol-5-yl)amino)-1-oxopropan-2-yl)-2-(4-fluoro-3,5-dimethylphenyl)-4-methyl-2,4,6,7-tetrahydro-5H-pyrazolo[4,3-c]pyridine-5-carboxylate

In a vial, sodium iodide (11.7 mg, 78.0 mol), cesium carbonate (127 mg, 390 mol), dimethylamine (5.27 mg, 117 mol), tert-butyl (4S)-3-(2-chloro-1-((4-fluoro-1-methyl-1H-indazol-5-yl)amino)-1-oxopropan-2-yl)-2-(4-fluoro-3,5-dimethylphenyl)-4-methyl-2,4,6,7-tetrahydro-5H-pyrazolo[4,3-c]pyridine-5-carboxylate (47.8 mg, 78.0 mol) were dissolved in MeCN (0.4 mL) and stirred at 50° C. for 30 minutes. The reaction was concentrated under reduced pressure and purified by silica gel column chromatography (0-10% MeOH in DCM) to provide the title compound. MS (ESI) m/z: 622.2 [M+H]+.

### Step 3: 2-(dimethylamino)-N-(4-fluoro-1-methyl-1H-indazol-5-yl)-2-((S)-2-(4-fluoro-3,5-dimethylphenyl)-4-methyl-4,5,6,7-tetrahydro-2H-pyrazolo[4,3-c]pyridin-3-yl)propenamide

This step was performed in a similar manner as in Example 305, step 6 to provide the title compound. MS (ESI) m/z: 522.2 [M+H]+.

### Step 4: (S)-2-(dimethylamino)-2-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)-N-(4-fluoro-1-methyl-1H-indazol-5-yl)propenamide or (R)-2-(dimethylamino)-2-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)-N-(4-fluoro-1-methyl-1H-indazol-5-yl)propenamide

This step was performed in a similar manner as in Example 305, step 7 to provide the title compounds. Purified by prep-HPLC 65-75% MeCN:H2O+0.1% formic acid. Isomer A: MS (ESI) m/z: 916.1 [M+H]+. Isomer B: MS (ESI) m/z: 916.0 [M+H]+.

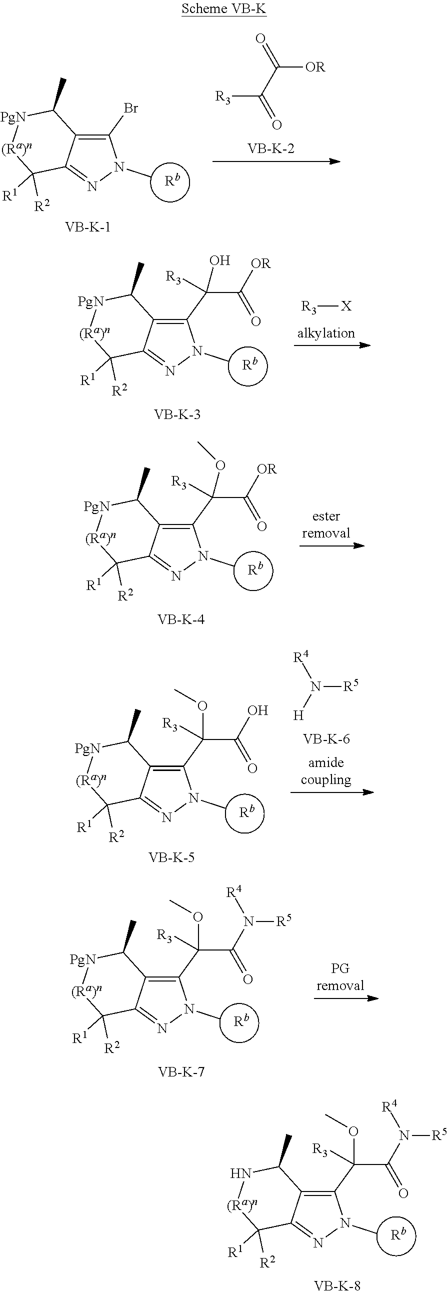

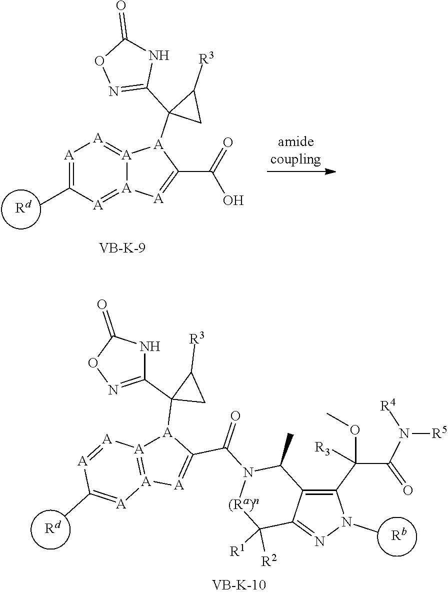

Compounds of formula (VB—K-10) are prepared from intermediate VB—K-1 by performing a metal-halogen exchange followed by trapping with alkyl pyruvate (VB—K-2). Alkylation of the alcohol with VB—K-3 affords ether VB—K-4. Removal of the ester (e.g., basic aqueous conditions) of VB—K-4 furnishes carboxylic acid VB—K-5. Subsequently, VB—K-5 is coupled with amine VB—K-6 to provide amide VB—K-7. Removal of the protecting group (e.g., acidic conditions) of VB—K-7 provides amine VB—K-8 that is then coupled with carboxylic acid VB—K-9 to provide compounds of formula (VB—K-10).

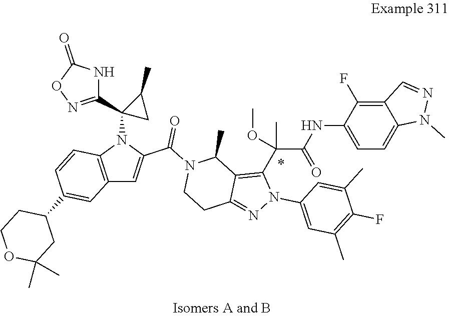 [SMILES: COC(C)(C(=O)Nc1ccc2c(cnn2C)c1F)c1c2c(nn1-c1cc(C)c(F)c(C)c1)CCN(C(=O)c1cc3cc([C@H]4CCOC(C)(C)C4)ccc3n1[C@@]1(c3noc(=O)[nH]3)C[C@@H]1C)[C@H]2C]

### (R)-2-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)-N-(4-fluoro-1-methyl-1H-indazol-5-yl)-2-methoxypropanamide (311A) or (S)-2-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)-N-(4-fluoro-1-methyl-1H-indazol-5-yl)-2-methoxypropanamide (311B)

### Step 1: tert-butyl (4S)-2-(4-fluoro-3,5-dimethylphenyl)-3-(2-hydroxy-1-methoxy-1-oxopropan-2-yl)-4-methyl-2,4,6,7-tetrahydro-5H-pyrazolo[4,3-c]pyridine-5-carboxylate

To a round bottom flask equipped with a stir bar was added tert-butyl (S)-3-bromo-2-(4-fluoro-3,5-dimethylphenyl)-4-methyl-2,4,6,7-tetrahydro-5H-pyrazolo[4,3-c]pyridine-5-carboxylate (3019 mg, 6.89 mmol). The flask was sealed, sparged with argon and THF (50 mL) was added. The flask was cooled to −78° C. and n-Butyllithium (706 mg, 6.89 mL, 1.6 molar, 11.0 mmol) was added dropwise. Three minutes later, methylpyruvate (1.76 g, 1.56 mL, 17.2 mmol) was added dropwise and the reaction was warmed to room temperature. The reaction was quenched with careful addition of sat. NH₄Cl solution and diluted with EtOAc. The mixture was extracted with EtOAc, and combined organic layers were washed with brine, dried with MgSO₄ and concentrated under reduced pressure. Purification was achieved by normal phase silica gel chromatography using 0-100% EtOAc in hexanes over 20 min to afford the product. MS (ESI) m/z: 462.2 [M+H]⁺.

### Step 2: tert-butyl (4S)-3-(1,2-dimethoxy-1-oxopropan-2-yl)-2-(4-fluoro-3,5-dimethylphenyl)-4-methyl-2,4,6,7-tetrahydro-5H-pyrazolo[4,3-c]pyridine-5-carboxylate

Silver (I) oxide (132 mg, 569 mol), tert-butyl (4S)-2-(4-fluoro-3,5-dimethylphenyl)-3-(2-hydroxy-1-methoxy-1-oxopropan-2-yl)-4-methyl-2,4,6,7-tetrahydro-5H-pyrazolo[4,3-c]pyridine-5-carboxylate (250 mg, 542 mol) and iodomethane (231 mg, 1.63 mmol) were added to a 20 mL vial and acetone (1 mL) was added. The suspension was stirred at 25° C. for 60 hours, at which time the suspension was filtered, and filtrates were concentrated under reduced pressure. Purification was achieved by normal phase silica gel chromatography using 0-100% EtOAc in hex over 15 min to afford the product. MS (ESI) m/z: 476.2 [M+H]⁺.

### Step 3: 2-((S)-5-(tert-butoxycarbonyl)-2-(4-fluoro-3,5-dimethylphenyl)-4-methyl-4,5,6,7-tetrahydro-2H-pyrazolo[4,3-c]pyridin-3-yl)-2-methoxypropanoic acid

In a 20 mL vial, tert-butyl (4S)-3-(1,2-dimethoxy-1-oxopropan-2-yl)-2-(4-fluoro-3,5-dimethylphenyl)-4-methyl-2,4,6,7-tetrahydro-5H-pyrazolo[4,3-c]pyridine-5-carboxylate (218 mg, 459 mol) was dissolved in THF (500 L). Lithium hydroxide (96.3 mg, 2.29 mmol) was dissolved in water (500 L) and added to the ester solution. The mixture was stirred at room temperature for six hours, at which time MeOH (500 L) was added and heated to 60° C. Two hours later, the reaction was cooled to room temperature and diluted with water, 1 N HCl solution and EtOAc. The mixture was extracted with EtOAc and combined organic layers were washed with brine, dried with MgSO₄ and concentrated under reduced pressure. MS (ESI) m/z: 462.2 [M+H]⁺.

### Step 4: tert-butyl (4S)-3-(1-((4-fluoro-1-methyl-1H-indazol-5-yl)amino)-2-methoxy-1-oxopropan-2-yl)-2-(4-fluoro-3,5-dimethylphenyl)-4-methyl-2,4,6,7-tetrahydro-5H-pyrazolo[4,3-c]pyridine-5-carboxylate

A vial was charged with 2-((S)-5-(tert-butoxycarbonyl)-2-(4-fluoro-3,5-dimethylphenyl)-4-methyl-4,5,6,7-tetrahydro-2H-pyrazolo[4,3-c]pyridin-3-yl)-2-methoxypropanoic acid (144 mg, 312 mol), 4-fluoro-1-methyl-1H-indazol-5-amine HCl (253 mg, 936 mol), 2-chloro-1-methyl-pyridin-1-ium iodide (87.7 mg, 343 mol), DCM (0.8 mL), and triethylamine (174 mg, 239 μL, 1.72 mmol). The reaction mixture was allowed to stir for 10 minutes at which time LCMS indicated complete consumption of starting material. The mixture was loaded directly onto a 12 g SiO₂ column eluting with 0-100% hexanes:EtOAc to yield the desired product. MS (ESI) m/z: 609.2 [M+H]⁺.

### Step 5: N-(4-fluoro-1-methyl-1H-indazol-5-yl)-2-((S)-2-(4-fluoro-3,5-dimethylphenyl)-4-methyl-4,5,6,7-tetrahydro-2H-pyrazolo[4,3-c]pyridin-3-yl)-2-methoxypropanamide

In a vial, tert-butyl (4S)-3-(1-((4-fluoro-1-methyl-1H-indazol-5-yl)amino)-2-methoxy-1-oxopropan-2-yl)-2-(4-fluoro-3,5-dimethylphenyl)-4-methyl-2,4,6,7-tetrahydro-5H-pyrazolo[4,3-c]pyridine-5-carboxylate (70 mg, 0.12 mmol) was treated with hydrogen chloride (42 mg, 0.29 mL, 4 molar, 1.2 mmol) in dioxane at 25° C. One hour later, LCMS analysis showed consumption of starting material and formation of product. The reaction was concentrated under reduced pressure and used directly in the next step. MS (ESI) m/z: 509.2 [M+H]⁺.

### Step 6: N-(4-fluoro-1-methyl-1H-indazol-5-yl)-2-((S)-2-(4-fluoro-3,5-dimethylphenyl)-4-methyl-4,5,6,7-tetrahydro-2H-pyrazolo[4,3-c]pyridin-3-yl)-2-methoxypropanamide

In a 4 mL vial, HATU (50 mg, 0.13 mmol), 5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carboxylic acid (54 mg, 0.13 mmol), N-(4-fluoro-1-methyl-1H-indazol-5-yl)-2-((S)-2-(4-fluoro-3,5-dimethylphenyl)-4-methyl-4,5,6,7-tetrahydro-2H-pyrazolo[4,3-c]pyridin-3-yl)-2-methoxypropanamide (61 mg, 0.12 mmol), diisopropylethylamine (0.16 g, 0.21 mL, 1.2 mmol) were dissolved in DMF (0.4 mL) and the vial was stirred at 25° C. overnight. The reaction mixture was filtered (Nalgene®, PTFE 0.45) and purified by reverse phase prep-HPLC (70-95% MeCN/water+0.1% formic acid modifier) followed by prep-SFC (Berger MGII SFC, Daicel ChiralPak®IC, 250 (L)×30 (ID) mm, 85 mL/min, 30% EtOH CO₂ modifier) to afford the title compounds as solids. NMR data MS (ESI) m/z: 902.4 [M+H]⁺.

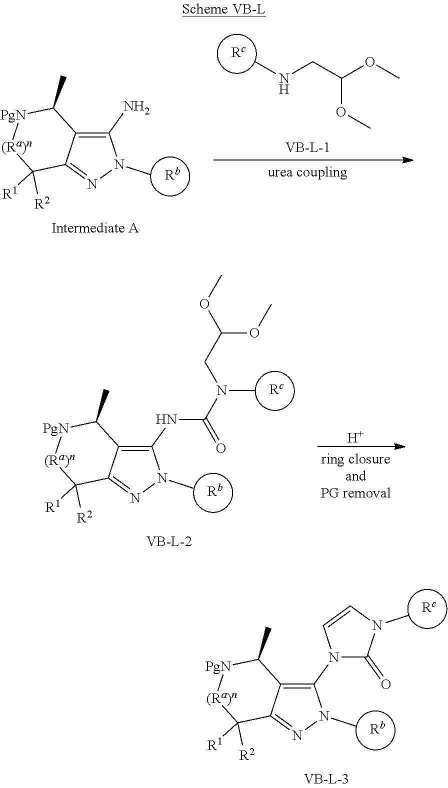

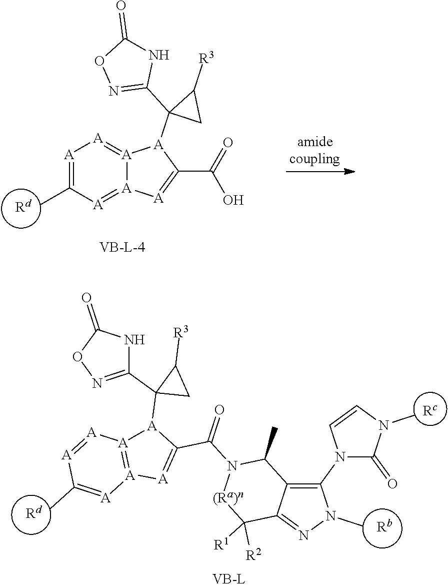

Compounds of formula VB-L are prepared from intermediate A by coupling with intermediate VB-L-1 in the presence of reagents like triphosgene to provide urea VB-L-2. Ring closure of VB-L-2 promoted by acidic conditions with spontaneous PG deprotection provides imidazol-2-one VB-L-3. Coupling with carboxylic acid VB-L-4 provides compounds of formula VB-L.

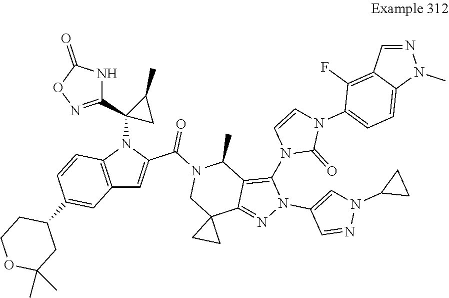 [SMILES: C[C@H]1c2c(nn(-c3cnn(C4CC4)c3)c2-n2ccn(-c3ccc4c(cnn4C)c3F)c2=O)C2(CC2)CN1C(=O)c1cc2cc([C@H]3CCOC(C)(C)C3)ccc2n1[C@@]1(c2noc(=O)[nH]2)C[C@@H]1C]

### 3-((1S,2S)-1-(2-((4′S)-2′-(1-cyclopropyl-1H-pyrazol-4-yl)-3′-(3-(4-fluoro-1-methyl-1H-indazol-5-yl)-2-oxo-2,3-dihydro-1H-imidazol-1-yl)-4′-methyl-2′,4′,5′,6′-tetrahydrospiro[cyclopropane-1,7′-pyrazolo[4,3-c]pyridine]-5′-carbonyl)-5-((S)-2,2-dimethyltetrahydro-2H-pyran-4-yl)-1H-indol-1-yl)-2-methylcyclopropyl)-1,2,4-oxadiazol-5(4H)-one (Scheme VB-L)

### Step 1: tert-butyl (S)-2′-(1-cyclopropyl-1H-pyrazol-4-yl)-3′-(3-(2,2-dimethoxyethyl)-3-(4-fluoro-1-methyl-1H-indazol-5-yl)ureido)-4′-methyl-2′,4′-dihydrospiro[cyclopropane-1,7′-pyrazolo[4,3-c]pyridine]-5′(6′H)-carboxylate

A solution of tert-butyl (S)-3′-amino-2′-(1-cyclopropyl-1H-pyrazol-4-yl)-4′-methyl-2′,4′-dihydrospiro[cyclopropane-1,7′-pyrazolo[4,3-c]pyridine]-5′(6′H)-carboxylate (80 mg, 0.21 mmol) in THF (0.5 mL) was added to solution of triphosgene (62 mg, 0.21 mmol) and DIPEA (0.16 g, 0.22 mL, 1.2 mmol) in THF (0.6 mL) at 0° C. The reaction mixture was then warmed to rt and stirred for 1 hour. A solution of N-(2,2-dimethoxyethyl)-4-fluoro-1-methyl-1H-indazol-5-amine (53 mg, 0.21 mmol) in THF (1.0 mL) was then added, followed by stirring at rt for 6 h. A saturated aqueous solution of NH₄Cl (5 mL) and EtOAc (10 mL) were added, the layers were mixed then separated, and the aqueous layer was back-extracted with EtOAc (10 mL×3). The combined organic layers were washed with brine (10 mL), dried over Na₂SO₄, filtered and concentrated under reduced pressure. The residue was purified by silica gel chromatography (0-100% EtOAc/hexanes) to provide the title compound. MS (ESI): m/z 664.4 [M+H]⁺.

### Step 2: 1-((4′S)-2′-(1-cyclopropyl-1H-pyrazol-4-yl)-4′-methyl-2′,4′,5′,6′-tetrahydrospiro[cyclopropane-1,7′-pyrazolo[4,3-c]pyridin]-3′-yl)-3-(4-fluoro-1-methyl-1H-indazol-5-yl)-1,3-dihydro-2H-imidazol-2-one

A mixture of (S)-2′-(1-cyclopropyl-1H-pyrazol-4-yl)-3′-(3-(2,2-dimethoxyethyl)-3-(4-fluoro-1-methyl-1H-indazol-5-yl)ureido)-4′-methyl-2′,4′-dihydrospiro[cyclopropane-1,7′-pyrazolo[4,3-c]pyridine]-5′(6′H)-carboxylate (63 mg, 0.095 mmol) and methanesulfonic acid (9.1 mg, 6.2 μL, 0.095 mmol) in THF (0.95 mL) was stirred at 60° C. for 17 h. Additional methanesulfonic acid (50 μL, 0.76 mmol) was added, followed by stirring at 60° C. for 3 h. After cooling to rt, a saturated aqueous solution of NaHCO₃ (5 mL) and EtOAc (10 mL) were added, the layers were mixed then separated, and the aqueous layer was back-extracted with EtOAc (10 mL×3). The combined organic layers were dried over Na₂SO₄, filtered and concentrated under reduced pressure to provide the title compound, which was used in the following step without further purification. MS (ESI): m/z 500.1 [M+H]⁺.

### Step 3: 3-((1S,2S)-1-(2-((4′S)-2′-(1-cyclopropyl-1H-pyrazol-4-yl)-3′-(3-(4-fluoro-1-methyl-1H-indazol-5-yl)-2-oxo-2,3-dihydro-1H-imidazol-1-yl)-4′-methyl-2′,4′,5′,6′-tetrahydrospiro[cyclopropane-1,7′-pyrazolo[4,3-c]pyridine]-5′-carbonyl)-5-((S)-2,2-dimethyltetrahydro-2H-pyran-4-yl)-1H-indol-1-yl)-2-methylcyclopropyl)-1,2,4-oxadiazol-5(4H)-one

A mixture of 1-((4′S)-2′-(1-cyclopropyl-1H-pyrazol-4-yl)-4′-methyl-2′,4′,5′,6′-tetrahydrospiro[cyclopropane-1,7′-pyrazolo[4,3-c]pyridin]-3′-yl)-3-(4-fluoro-1-methyl-1H-indazol-5-yl)-1,3-dihydro-2H-imidazol-2-one (36 mg, 0.072 mmol), 5-(2,2-dimethyltetrahydro-2H-pyran-4-yl)-1-((2S)-2-methyl-1-(5-oxo-4,5-dihydro-1,2,4-oxadiazol-3-yl)cyclopropyl)-1H-indole-2-carboxylic acid (33 mg, 0.079 mmol) and HATU (33 mg, 0.086 mmol) in DMF (0.90 mL was treated with DIPEA (28 mg, 38 μL, 0.22 mmol) and stirred at rt for 22 h. A saturated aqueous solution of NH₄Cl (4 mL) and EtOAc (5 mL) were added, and the layers were mixed and then separated. The aqueous layer was back-extracted with EtOAc (10 mL×3). The combined organic layers were washed with brine (7 mL×3), dried over Na₂SO₄, filtered and concentrated under reduced pressure. The residue was purified by silica gel chromatography (0-30% MeOH+1% NH40H modifier in DCM) to provide the title compound. ¹H-NMR (CD₃CN, 400 MHz, Rotamers) δ_{H} 11.43 (1H, s), 8.10 (1H, d, J=39.6 Hz), 7.68 (1H, d, J=29.0 Hz), 7.54-7.39 (3H, m), 7.28-7.15 (2H, m), 6.81-6.77 (1H, m), 6.63 (1H, t, J=2.9 Hz), 6.43 (1H, d, J=3.2 Hz), 5.78 (0.4H, q, J=6.3 Hz), 5.33 (0.6H, q, J=6.5 Hz), 4.19 (1H, d, J=13.5 Hz), 4.09-4.02 (3H, m), 3.87-3.72 (2H, m), 3.68-3.61 (1H, m), 3.06 (1H, t, J=12.7 Hz), 2.30-2.23 (1H, m), 2.11-2.10 (1H, m), 1.83-1.65 (3H, m), 1.62 (2H, d, J=6.9 Hz), 1.58-1.49 (3H, m), 1.48-1.40 (2H, m), 1.38-1.27 (7H, m), 1.19 (3H, s), 1.15 (1H, d, J=6.2 Hz), 1.12-0.93 (5H, m), 0.90-0.81 (1H, m). ¹⁹F NMR (CH₃CN-d3, 376 MHz): δ_{F}: ¹⁹F-NMR (CD₃CN, 376 MHz, Rotamers) 6-127.6 (1F, d, J=6.7 Hz), −127.6 (1F, d, J=6.1 Hz). MS (ESI): m/z 893.5 [M+H]⁺.

The following examples in Table 27 were prepared according to scheme VB-L using the procedures outlined in the synthesis of example 312.

TABLE 27

| | | | MS |
|---|---|---|---|
| Ex. | Structure | Name | (M + 1) |
| 313 | 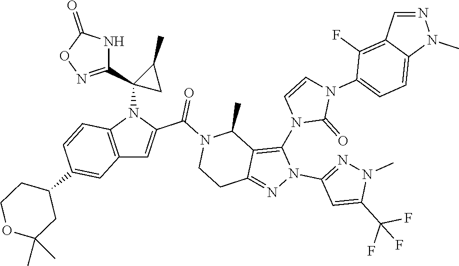 [SMILES: C[C@H]1c2c(nn(-c3cc(C(F)(F)F)n(C)n3)c2-n2ccn(-c3ccc4c(cnn4C)c3F)c2=O)CCN1C(=O)c1cc2cc([C@H]3CCOC(C)(C)C3)ccc2n1[C@@]1(c2noc(=O)[nH]2)C[C@@H]1C] | 3-((1S,2S)-1-(5-((S)-2,2- dimethyltetrahydro-2H-pyran-4- yl)-2-((4S)-3-(3-(4-fluoro-1- methyl-1H-indazol-5-yl)-2-oxo- 2,3-dihydro-1H-imidazol-1-yl)-4- methyl-2-(1-methyl-5- (trifluoromethyl)-1H-pyrazol-3- yl)-4,5,6,7-tetrahydro-2H- pyrazolo[4,3-c]pyridine-5- carbonyl)-1H-indol-1-yl)-2- methylcyclopropyl)-1,2,4- oxadiazol-5(4H)-one | 909.4 |
| 314 | 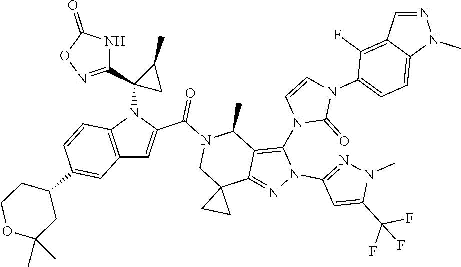 [SMILES: C[C@H]1c2c(nn(-c3cc(C(F)(F)F)n(C)n3)c2-n2ccn(-c3ccc4c(cnn4C)c3F)c2=O)C2(CC2)CN1C(=O)c1cc2cc([C@H]3CCOC(C)(C)C3)ccc2n1[C@@]1(c2noc(=O)[nH]2)C[C@@H]1C] | 3-((1S,2S)-1-(5-((S)-2,2- dimethyltetrahydro-2H-pyran-4- yl)-2-((4′S)-3′-(3-(4-fluoro-1- methyl-1H-indazol-5-yl)-2-oxo- 2,3-dihydro-1H-imidazol-1-yl)-4'- methyl-2′-(1-methyl-5- (trifluoromethyl)-1H-pyrazol-3- yl)-2′,4′,5′,6′- tetrahydrospiro[cyclopropane-1,7′- pyrazolo[4,3-c]pyridine]-5′- carbonyl)-1H-indol-1-yl)-2- methylcyclopropyl)-1,2,4- oxadiazol-5(4H)-one | 935.5 |
| 315 | 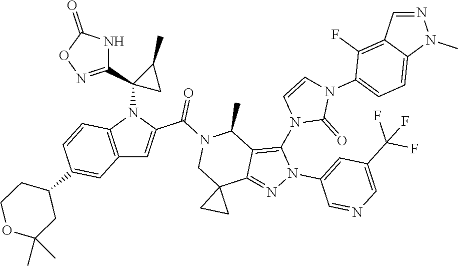 [SMILES: C[C@H]1c2c(nn(-c3cncc(C(F)(F)F)c3)c2-n2ccn(-c3ccc4c(cnn4C)c3F)c2=O)C2(CC2)CN1C(=O)c1cc2cc([C@H]3CCOC(C)(C)C3)ccc2n1[C@@]1(c2noc(=O)[nH]2)C[C@@H]1C] | 3-((1S,2S)-1-(5-((S)-2,2- dimethyltetrahydro-2H-pyran-4- yl)-2-((4′S)-3′-(3-(4-fluoro-1- methyl-1H-indazol-5-yl)-2-oxo- 2,3-dihydro-1H-imidazol-1-yl)-4'- methyl-2′-(5- (trifluoromethyl)pyridin-3-yl)- 2′,4′,5′,6′- tetrahydrospiro[cyclopropane-1,7′- pyrazolo[4,3-c]pyridine]-5′- carbonyl)-1H-indol-1-yl)-2- methylcyclopropyl)-1,2,4- oxadiazol-5(4H)-one | 932.6 |

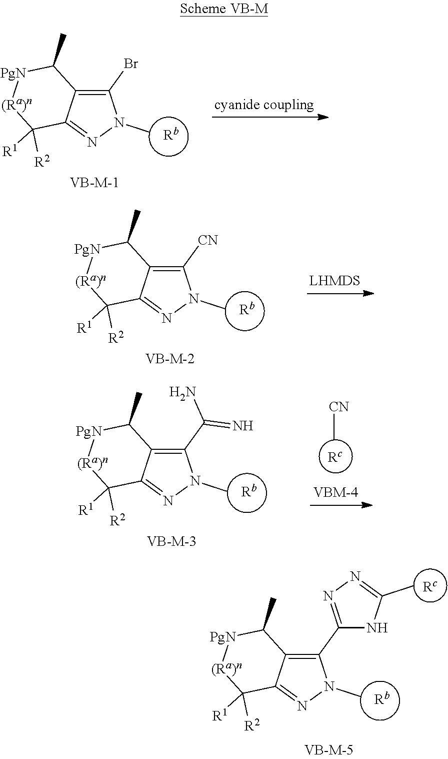

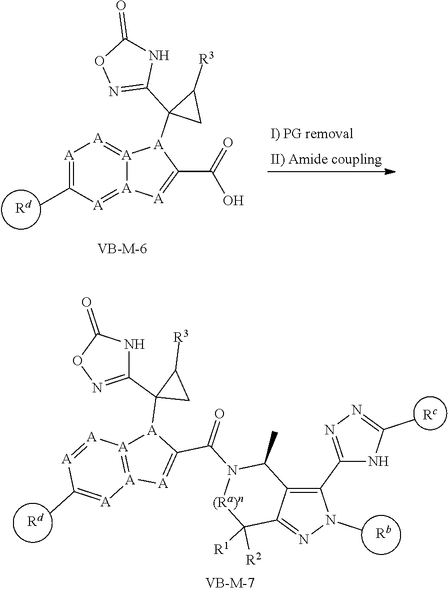

Compounds of formula VB-M-7 are prepared from Intermediate VB-M-1 through metal-mediated cyanide coupling to provide cyano VB-M-2. Addition of LHMDS to VB-M-2 provides VB-M-3. Addition of intermediates such as VB-M-4 in the presence of reagents like Cu(OAc)₂ to promote spontaneous oxidation provides triazoles of the form VB-M-5. Acid-mediated PG removal followed by coupling with carboxylic acid VB-M-6 provides compounds of formula VB-M-7.

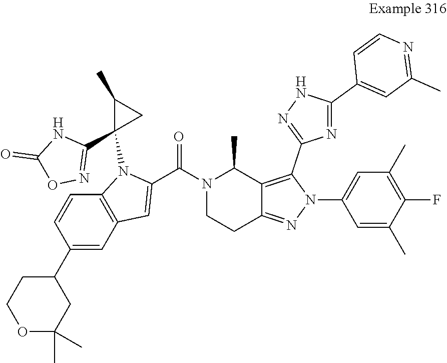 [SMILES: Cc1cc(-c2nc(-c3c4c(nn3-c3cc(C)c(F)c(C)c3)CCN(C(=O)c3cc5cc(C6CCOC(C)(C)C6)ccc5n3[C@@]3(c5noc(=O)[nH]5)C[C@@H]3C)[C@H]4C)n[nH]2)ccn1]

### 3-[(1S,2S)-1-[5-[(4S)-2,2-dimethyltetrahydropyran-4-yl]-2-[(4S)-2-(4-fluoro-3,5-dimethyl-phenyl)-4-methyl-3-[5-(2-methyl-4-pyridyl)-1H-1,2,4-triazol-4-ium-3-yl]-6,7-dihydro-4H-pyrazolo[4,3-c]pyridine-5-carbonyl]indol-1-yl]-2-methyl-cyclopropyl]-4H-1,2,4-oxadiazol-5-one formate

### Step 1: tert-butyl (S)-3-cyano-2-(4-fluoro-3,5-dimethylphenyl)-4-methyl-2,4,6,7-tetrahydro-5H-pyrazolo[4,3-c]pyridine-5-carboxylate

To a solution of tert-butyl (S)-3-bromo-2-(4-fluoro-3,5-dimethylphenyl)-4-methyl-2,4,6,7-tetrahydro-5H-pyrazolo[4,3-c]pyridine-5-carboxylate (200 mg, 0.456 mmol), potassium ferrocyanide (42.0 mg, 0.114 mmol) and sodium carbonate (48.4 mg, 0.456 mmol) in NMP (3 mL) was added chloro(2-dicyclohexylphosphino-2′,4′,6′-triisopropyl-1,1′-biphenyl)[2-(2′-amino-1,1′-biphenyl)]palladium(II) (35.9 mg, 0.046 mmol), and the resulting mixture was stirred at 100° C. for 18 h. The mixture was extracted with EtOAc (3×8 mL) and H₂O (8 mL). The combined organic extracts were washed with brine (20 mL), dried over anhydrous Na₂SO₄, filtered and concentrated under reduced pressure. The residue was purified by preparative HPLC to provide the title compound. MS (ESI) m/z: 385.0 [M+H]+.

### Step 2: tert-butyl (S)-3-carbamimidoyl-2-(4-fluoro-3,5-dimethylphenyl)-4-methyl-2,4,6,7-tetrahydro-5H-pyrazolo[4,3-c]pyridine-5-carboxylate

To a solution of tert-butyl (S)-3-cyano-2-(4-fluoro-3,5-dimethylphenyl)-4-methyl-2,4,6,7-tetrahydro-5H-pyrazolo[4,3-c]pyridine-5-carboxylate (230.00 mg, 598.24 mol) in THF (5 mL) was added lithium bis(trimethylsilyl)amide (1M, 3.58 mL, 3.58 mmol) at 0° C. The reaction mixture was stirred at 30° C. under the protection of N₂ for 16 hours. The reaction solution was quenched with HCl and was then concentrated under reduced pressure. The residue was purified by preparative HPLC to provide the title compound. MS (ESI) m/z: 402.2 [M+H]⁺.

### Step 3: tert-butyl (S)-2-(4-fluoro-3,5-dimethylphenyl)-4-methyl-3-(5-(2-methylpyridin-4-yl)-4H-1,2,4-triazol-3-yl)-2,4,6,7-tetrahydro-5H-pyrazolo[4,3-c]pyridine-5-carboxylate

To a solution of tert-butyl (S)-3-carbamimidoyl-2-(4-fluoro-3,5-dimethylphenyl)-4-methyl-2,4,6,7-tetrahydro-5H-pyrazolo[4,3-c]pyridine-5-carboxylate (20.53 mg, 51.13 mol), 2-methylisonicotinonitrile (6.041 mg, 51.13 mol) and Na₂CO₃ (21.68 mg, 204.5 mol) in PhCH₃ (1 mL) was added copper diacetate (9.288 mg, 51.13 mol) and the resulting mixture was stirred at 110° C. under O₂ for 16 h. The mixture was extracted with EtOAc (3×5 mL) and H₂O (8 mL). The combined organic extracts were dried over anhydrous Na₂SO₄, filtered and concentrated under reduced pressure. The residue was purified by preparative HPLC to provide the title compound. MS (ESI) m/z: 518.2 [M+H]⁺.

### Step 4: (S)-2-(4-fluoro-3,5-dimethylphenyl)-4-methyl-3-(3-(2-methylpyridin-4-yl)-1H-1,2,4-triazol-5-yl)-4,5,6,7-tetrahydro-2H-pyrazolo[4,3-c]pyridine

To a solution of tert-butyl (S)-2-(4-fluoro-3,5-dimethylphenyl)-4-methyl-3-(3-(2-methylpyridin-4-yl)-1H-1,2,4-triazol-5-yl)-2,4,6,7-tetrahydro-5H-pyrazolo[4,3-c]pyridine-5-carboxylate (21.1 mg, 40.8 mol) in dioxane (1 mL) was added 2M HCl-dioxane (2M, 20.4 L, 40.8 mol), and the resulting mixture was stirred at 25° C. for 2 hour. The reaction was concentration to provide the title compound, which was used in the next step without further purification. MS (ESI) m/z: 418.2 [M+H]⁺.

### Step 5: 3-((1S,2S)-1-(5-((S)-2,2-dimethyltetrahydro-2H-pyran-4-yl)-2-((S)-2-(4-fluoro-3,5-dimethylphenyl)-4-methyl-3-(5-(2-methylpyridin-4-yl)-1H-1,2,4-triazol-3-yl)-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one

To a solution of (S)-2-(4-fluoro-3,5-dimethylphenyl)-4-methyl-3-(3-(2-methylpyridin-4-yl)-1H-1,2,4-triazol-5-yl)-4,5,6,7-tetrahydro-2H-pyrazolo[4,3-c]pyridine (16.5 mg, 39.5 mol), 5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carboxylic acid (19.00 mg, 46.18 mol), DIPEA (34.4 μL, 98 mol) in DMF (1 mL) was added HATU (22.5 mg, 59.3 mol), and the resulting mixture was stirred at 25° C. for 16 hour. The reaction mixture was filtered and the pH was adjusted with TFA. The crude product was purified by preparative HPLC to provide the title compound. MS (ESI) m/z: 811.4 [M+H]⁺. ¹H NMR (400 MHz, acetonitrile-d₃) δ 11.58-11.43 (m, 1H), 8.70-8.47 (m, 1H), 8.27-8.13 (m, 1H), 7.95 (s, 1H), 7.57-7.50 (m, 2H), 7.44 (d, J=8.2 Hz, 1H), 7.28 (dd, J=1.4, 8.3 Hz, 1H), 7.22 (d, J=10.1 Hz, 1H), 7.15-7.00 (m, 2H), 6.90-6.81 (m, 1H), 6.03 (q, J=6.9 Hz, 1H), 5.68-5.58 (m, 1H), 4.81 (s, 1H), 4.47-4.37 (m, 1H), 3.78-3.71 (m, 2H), 3.13 (s, 1H), 3.10 (br s, 1H), 2.78-2.76 (m, 2H), 2.67 (s, 1H), 2.26-2.26 (m, 3H), 2.23 (d, J=1.9 Hz, 3H), 2.11 (td, J=2.5, 4.9 Hz, 1H), 1.85 (d, J=6.7 Hz, 1H), 1.81 (br d, J=5.2 Hz, 1H), 1.77 (td, J=2.5, 4.9 Hz, 1H), 1.73-1.68 (m, 2H), 1.62 (d, J=6.7 Hz, 3H), 1.59-1.55 (m, 1H), 1.30-1.27 (m, 3H), 1.22-1.14 (m, 6H), 1.01 (d, J=6.3 Hz, 1H)

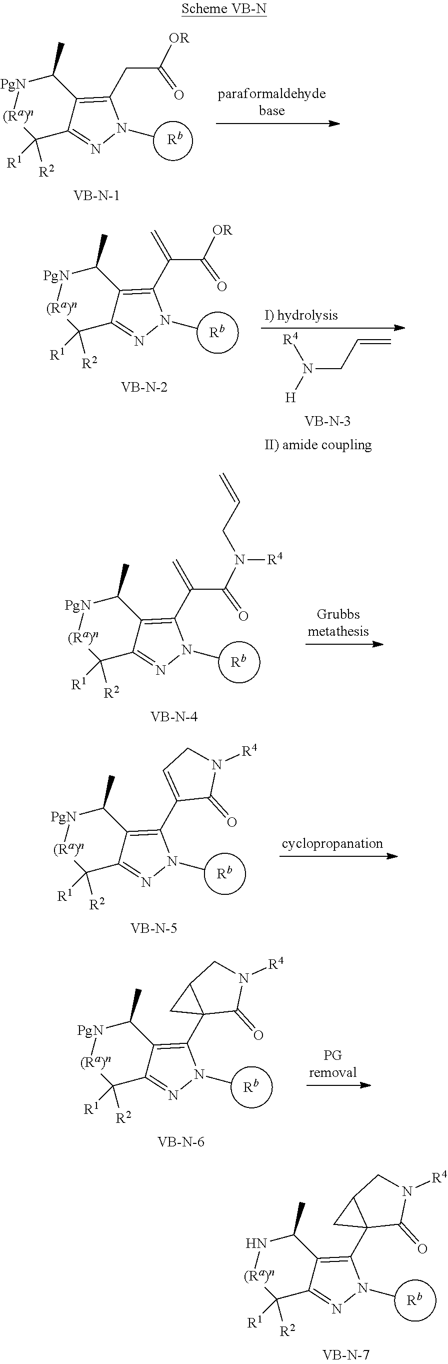

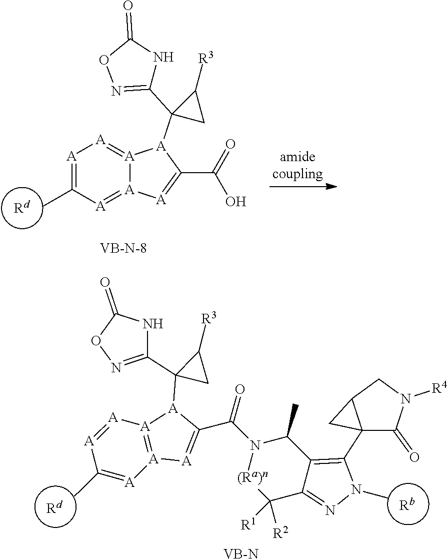

Compounds of formula (VB—N) are prepared from VB—N-1 by reacting with paraformaldehyde and base to provide exomethylene VB—N-2. Coupling with an amine partner provides VB—N-4 which can undergo a Ru-catalyzed metathesis reaction to provide VB—N-5. Photo-mediated cyclopropanation yields two isomers of VB—N-6 which can then undergo protecting group removal (e.g., acidic conditions) to provide amine VB—N-7 that is then coupled with carboxylic acid VB—N-8 to provide compounds of formula (VB—N). Alternatively, the order of steps for some examples may be varied to facilitate the syntheses of the title compounds of formula (VB—N).

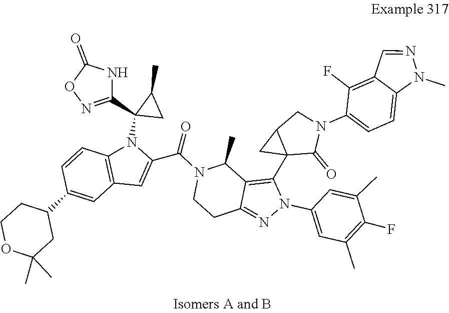

### 3-((1S,2S)-1-(5-((S)-2,2-dimethyltetrahydro-2H-pyran-4-yl)-2-((S)-3-((1R,5S)-3-(4-fluoro-1-methyl-1H-indazol-5-yl)-2-oxo-3-azabicyclo[3.1.0]hexan-1-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one (317A) and 3-((1S,2S)-1-(5-((S)-2,2-dimethyltetrahydro-2H-pyran-4-yl)-2-((S)-3-((1S,5R)-3-(4-fluoro-1-methyl-1H-indazol-5-yl)-2-oxo-3-azabicyclo[3.1.0]hexan-1-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one (317B)

### Step 1: tert-butyl (S)-2-(4-fluoro-3,5-dimethylphenyl)-3-(3-methoxy-3-oxoprop-1-en-2-yl)-4-methyl-2,4,6,7-tetrahydro-5H-pyrazolo[4,3-c]pyridine-5-carboxylate

To a vial containing tert-butyl (S)-2-(4-fluoro-3,5-dimethylphenyl)-3-(2-methoxy-2-oxoethyl)-4-methyl-2,4,6,7-tetrahydro-5H-pyrazolo[4,3-c]pyridine-5-carboxylate (300 mg, 695 μmol) was added paraformaldehyde (41.8 mg, 0.39 mmol) and potassium carbonate (96.1 mg, 695 μmol). The solids were suspended in DMF (1 mL) and heated to 100° C. for 2 hours. The reaction mixture was cooled to 20° C. and purified by silica gel chromatography (hexanes/EtOAc: 1/0 to 0/1) to provide the title compound. LCMS m/z[M+H]: 444.2.

### Step 2: (S)-2-(5-(tert-butoxycarbonyl)-2-(4-fluoro-3,5-dimethylphenyl)-4-methyl-4,5,6,7-tetrahydro-2H-pyrazolo[4,3-c]pyridin-3-yl)acrylic acid

To a solution of tert-butyl (S)-2-(4-fluoro-3,5-dimethylphenyl)-3-(2-methoxy-2-oxoethyl)-4-methyl-2,4,6,7-tetrahydro-5H-pyrazolo[4,3-c]pyridine-5-carboxylate (45 mg, 0.10 mmol) in THF (3 mL) was added lithium hydroxide hydrate (22 mg, 0.52 mmol) followed by water (3 mL). The reaction was stirred at 20° C. for 18 hours. To the reaction mixture was added 1N aqueous HCl to reach a pH of 0. Ethyl acetate (20 mL) was added, and the layers were separated. The aqueous layer was extracted with EtOAc (3×20 mL). The combined organic layers were dried over sodium sulfate, filtered, and concentrated under reduced pressure. The crude mixture was taken onto the next step without purification. LCMS m/z[M+H]: 418.4.

### Step 3: tert-butyl (S)-3-(3-(allyl(4-fluoro-1-methyl-1H-indazol-5-yl)amino)-3-oxoprop-1-en-2-yl)-2-(4-fluoro-3,5-dimethylphenyl)-4-methyl-2,4,6,7-tetrahydro-5H-pyrazolo[4,3-c]pyridine-5-carboxylate

To a solution of (S)-2-(5-(tert-butoxycarbonyl)-2-(4-fluoro-3,5-dimethylphenyl)-4-methyl-4,5,6,7-tetrahydro-2H-pyrazolo[4,3-c]pyridin-3-yl)acrylic acid (385 mg, 896 μmol) and N-allyl-4-fluoro-1-methyl-1H-indazol-5-amine hydrogen chloride (325 mg, 1.34 mmol) in DCM (8 mL) was added triethylamine (375 μL, 2.69 mmol) followed by 2-chloro-1-methyl-pyridin-1-ium iodide (275 mg, 1.08 mmol). The reaction mixture was stirred at 23° C. for 2 hours. The crude mixture was purified by C18 reverse phase column chromatography (5-100% MeCN/water+0.1% FA modifier) to afford the title compound. LCMS m/z[M+H]: 617.4

### Step 4: tert-butyl (S)-3-(1-(4-fluoro-1-methyl-1H-indazol-5-yl)-2-oxo-2,5-dihydro-1H-pyrrol-3-yl)-2-(4-fluoro-3,5-dimethylphenyl)-4-methyl-2,4,6,7-tetrahydro-5H-pyrazolo[4,3-c]pyridine-5-carboxylate

To a solution of tert-butyl (S)-3-(3-(allyl(4-fluoro-1-methyl-1H-indazol-5-yl)amino)-3-oxoprop-1-en-2-yl)-2-(4-fluoro-3,5-dimethylphenyl)-4-methyl-2,4,6,7-tetrahydro-5H-pyrazolo[4,3-c]pyridine-5-carboxylate (351 mg, 569 mol) in toluene (5 mL) was added benzylidene(dichloro)(1,3-dimesityl-2-imidazolidinylidene)ruthenium—tricyclohexylphosphine (48.3 mg, 56.9 μmol). The reaction mixture was heated to 80° C. for 12 hours. After cooling to 25° C., the reaction mixture was filtered through a Celite® pad and washed with DCM (15 mL). The filtrate was washed with a saturated aqueous solution of NH₄Cl (5 mL). The aqueous layer was extracted with DCM (10 mL×2) and the combined organic layers were washed with brine (10 mL), dried over Na₂SO₄, filtered and concentrated under reduced pressure. The residue was purified by reverse phase column chromatography (5-100% MeCN/water+0.1% FA modifier) to afford the title compound. LCMS m/z[M+H]: 589.3

### Step 5: tert-butyl (S)-3-((1R,5S)-3-(4-fluoro-1-methyl-1H-indazol-5-yl)-2-oxo-3-azabicyclo[3.1.0]hexan-1-yl)-2-(4-fluoro-3,5-dimethylphenyl)-4-methyl-2,4,6,7-tetrahydro-5H-pyrazolo[4,3-c]pyridine-5-carboxylate and tert-butyl (S)-3-((1S,5R)-3-(4-fluoro-1-methyl-1H-indazol-5-yl)-2-oxo-3-azabicyclo[3.1.0]hexan-1-yl)-2-(4-fluoro-3,5-dimethylphenyl)-4-methyl-2,4,6,7-tetrahydro-5H-pyrazolo[4,3-c]pyridine-5-carboxylate

To a solution of tert-butyl (S)-3-(1-(4-fluoro-1-methyl-1H-indazol-5-yl)-2-oxo-2,5-dihydro-1H-pyrrol-3-yl)-2-(4-fluoro-3,5-dimethylphenyl)-4-methyl-2,4,6,7-tetrahydro-5H-pyrazolo[4,3-c]pyridine-5-carboxylate (100 mg, 170 μmol) in DMSO was added 4CzIPN (13.4 mg, 17.0 μmol) and triethylammonium bis(catecholato)iodomethyl silicate (98.2 mg, 255 μmol). The resultant mixture was sparged with argon for 15 minutes. The vessel was then sealed and irradiated with 2 blue LED Kessil lamps (456 nm) for 16 hours. Additional 4CzIPN (13.4 mg, 17.0 μmol) and triethylammonium bis(catecholato)iodomethyl silicate (98.2 mg, 255 μmol) were added, and the reaction mixture was irradiated with 2 blue LED Kessil lamps (440 nm) for another 16 hours. The residue was purified by reverse phase column chromatography (30-90% MeCN/water) to afford the separated title compounds. Each diastereomer was then purified by silica gel column chromatography. Both products were purified separately using silica gel column chromatography (DCM/EtOAc: 1/0 to 3/20, 0.5% Et₃N) to provide the title diastereomers. Isomer A: LCMS m/z[M+H]: 603.3. Isomer B: LCMS m/z[M+H]: 603.3.

### Step 6: 3-((1S,2S)-1-(5-((S)-2,2-dimethyltetrahydro-2H-pyran-4-yl)-2-((S)-3-((1R,5S)-3-(4-fluoro-1-methyl-1H-indazol-5-yl)-2-oxo-3-azabicyclo[3.1.0]hexan-1-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one and 3-((1S,2S)-1-(5-((S)-2,2-dimethyltetrahydro-2H-pyran-4-yl)-2-((S)-3-((1S,5R)-3-(4-fluoro-1-methyl-1H-indazol-5-yl)-2-oxo-3-azabicyclo[3.1.0]hexan-1-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one

These steps were performed in a similar fashion as described for example 1, step 5 for each isomer separately to provide the title compounds. Data for isomer A: ¹H NMR (400 MHz, CD₃CN): δ 8.06-7.92 (1H, m), 7.54-7.44 (2H, m), 7.35-7.21 (2H, m), 7.17-7.04 (3H, m), 6.87-6.75 (1H, m), 5.76-5.70 (0.8H, m), 5.26-5.23 (0.2H, m), 4.78-4.74 (0.2H, m), 4.39-4.32 (0.8H, m), 4.05-3.96 (3H, m), 3.79-3.43 (4H, m), 3.10-2.81 (4H, m), 2.60-2.43 (2H, m), 2.36-2.24 (2H, m), 1.83-1.43 (1OH, m), 1.41-1.05 (13H, m), 1.02-0.77 (2H, m) MS (ESI) m/z: 896.5 [M+H]+. Data for isomer B: ¹H NMR (400 MHz, CD₃CN): δ 8.05-8.01 (1H, m), 7.52-7.48 (4H, m), 7.39-7.14 (4H, m), 6.82-6.80 (1H, m), 5.79-5.76 (0.8H, m), 5.23-5.49 (0.2H, m), 4.64-4.86 (0.2H, m), 4.37-4.33 (0.8H, m), 4.23-4.18 (0.8H, m), 4.05-4.00 (3.2H, m), 3.76-3.48 (4H, m), 3.09-2.84 (3.2H, m), 2.49-2.44 (0.8H, m), 2.33-2.23 (3H, m), 1.81-1.36 (9H, m), 1.35-1.04 (11H, m), 0.96-0.78 (4H, m).

The following examples in Table ZY were prepared according to Scheme VB—N using the procedures outlined in the synthesis of example 317.

TABLE ZY

| | | | MS |
|---|---|---|---|
| Ex. | Structure | Name | (M+1) |
| 409 | 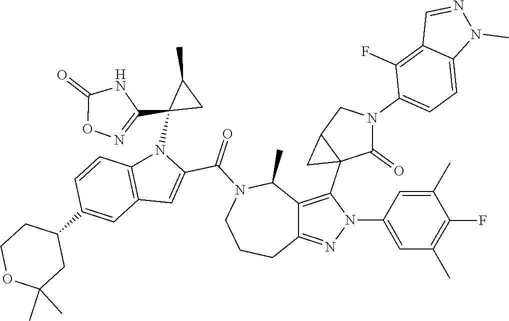 [SMILES: Cc1cc(-n2nc3c(c2C24CC2CN(c2ccc5c(cnn5C)c2F)C4=O)[C@H](C)N(C(=O)c2cc4cc([C@H]5CCOC(C)(C)C5)ccc4n2[C@@]2(c4noc(=O)[nH]4)C[C@@H]2C)CCC3)cc(C)c1F] | 3-((1S,2S)-1-(5-((S)-2,2- dimethyltetrahydro-2H-pyran-4-yl)- 2-((S)-3-((1S,5R)-3-(4-fluoro-1- methyl-1H-indazol-5-yl)-2-oxo-3- azabicyclo[3.1.0]hexan-1-yl)-2-(4- fluoro-3,5-dimethylphenyl)-4- methyl-2,4,5,6,7,8-hexahydropyrazolo [4,3-c]azepine-5-carbonyl)-1H-indol- 1-yl)-2-methylcyclopropyl)-1,2,4- oxadiazol-5(4H)-one OR 3-((1S,2S)- 1-(5-((S)-2,2-dimethyltetrahydro-2H- pyran-4-yl)-2-((S)-3-((1R,5S)-3-(4- fluoro-1-methyl-1H-indazol-5-yl)-2- oxo-3-azabicyclo[3.1.0]hexan-1-yl)- 2-(4-fluoro-3,5-dimethylphenyl)-4- methyl-2,4,5,6,7,8-hexahydropyrazolo [4,3-c]azepine-5-carbonyl)-1H-indol- 1-yl)-2-methylcyclopropyl)-1,2,4- oxadiazol-5(4H)-one | 910.4 |
| 410 | 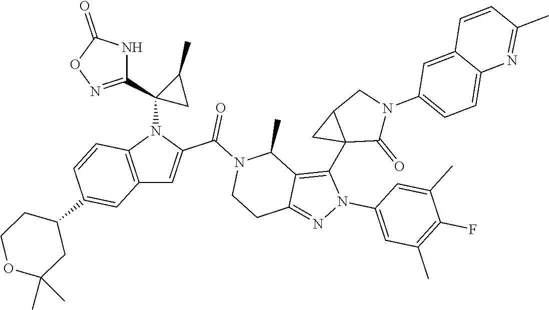 [SMILES: Cc1ccc2cc(N3CC4CC4(c4c5c(nn4-c4cc(C)c(F)c(C)c4)CCN(C(=O)c4cc6cc([C@H]7CCOC(C)(C)C7)ccc6n4[C@@]4(c6noc(=O)[nH]6)C[C@@H]4C)[C@H]5C)C3=O)ccc2n1] | 3-((1S,2S)-1-(5-((S)-2,2- dimethyltetrahydro-2H-pyran-4-yl)- 2-((S)-2-(4-fluoro-3,5-dimethylphenyl)- 4-methyl-3-((1S,5R)-3-(2- methylquinolin-6-yl)-2-oxo-3- azabicyclo[3.1.0]hexan-1-yl)- 4,5,6,7-tetrahydro-2H-pyrazolo[4,3-c] pyridine-5-carbonyl)-1H-indol-1-yl)-2- methylcyclopropyl)-1,2,4-oxadiazol- 5(4H)-one OR 3-((1S,2S)-1-(5-((S)- 2,2-dimethyltetrahydro-2H-pyran-4- yl)-2-((S)-2-(4-fluoro-3,5- dimethylphenyl)-4-methyl-3- ((1R,5S)-3-(2-methylquinolin-6-yl)- 2-oxo-3-azabicyclo[3.1.0]hexan-1- yl)-4,5,6,7-tetrahydro-2H- pyrazolo[4,3-c]pyridine-5-carbonyl)- 1H-indol-1-yl)-2-methylcyclopropyl)- 1,2,4-oxadiazol-5(4H)-one | 889.5 |

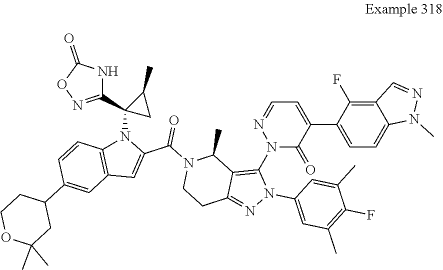 [SMILES: Cc1cc(-n2nc3c(c2-n2nccc(-c4ccc5c(cnn5C)c4F)c2=O)[C@H](C)N(C(=O)c2cc4cc(C5CCOC(C)(C)C5)ccc4n2[C@@]2(c4noc(=O)[nH]4)C[C@@H]2C)CC3)cc(C)c1F]

### 3-((1S,2S)-1-(5-(2,2-dimethyltetrahydro-2H-pyran-4-yl)-2-((4S)-3-(5-(4-fluoro-1-methyl-1H-indazol-5-yl)-6-oxopyridazin-1(6H)-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one

### Step 1: (S)-4,5-dichloro-2-(2-(4-fluoro-3,5-dimethylphenyl)-4-methyl-4,5,6,7-tetrahydro-2H-pyrazolo[4,3-c]pyridin-3-yl)pyridazin-3(2H)-one

To a solution of di-tert-butyl (S)-1-(5-(tert-butoxycarbonyl)-2-(4-fluoro-3,5-dimethylphenyl)-4-methyl-4,5,6,7-tetrahydro-2H-pyrazolo[4,3-c]pyridin-3-yl)hydrazine-1,2-dicarboxylate (755 mg, 1.28 mmol) in 12% HCl aq. (5 mL) was added (Z)-2,3-dichloro-4-oxobut-2-enoic acid (216 mg, 1.28 mmol), and the reaction mixture was stirred for 16 hours at 120° C. The reaction was then lyophilized to provide the title compound. LCMS m/z[M+H]: 423.8.

### Step 2: tert-butyl (S)-3-(4,5-dichloro-6-oxopyridazin-1(6H)-yl)-2-(4-fluoro-3,5-dimethylphenyl)-4-methyl-2,4,6,7-tetrahydro-5H-pyrazolo[4,3-c]pyridine-5-carboxylate

To a solution of (S)-4,5-dichloro-2-(2-(4-fluoro-3,5-dimethylphenyl)-4-methyl-4,5,6,7-tetrahydro-2H-pyrazolo[4,3-c]pyridin-3-yl)pyridazin-3(2H)-one (500 mg, 1.18 mmol) in THF (3 mL) and water (3 mL) was added Na₂CO₃ (188 mg, 1.78 mmol) and Boc₂O (0.275 mL, 1.18 mmol). The resultant mixture was stirred at 25° C. for 1.5 hours. The reaction mixture was diluted with ethyl acetate (5 mL) and aqueous 1 M HCl (3 mL). The organic layer was separated, and the aqueous phase was extracted with ethyl acetate (3×5 mL). The organic layers were combined and washed with brine, dried over sodium sulfate, and concentrated under reduced pressure. The crude residue was purified by silica gel chromatography (Pet. Ether/EtOAc: 1/0 to 65/35) to provide the title compound. LCMS m/z[M+H]: 523.7

### Step 3: tert-butyl (4S)-3-(4-chloro-5-(4-fluoro-1-methyl-1H-indazol-5-yl)-6-oxopyridazin-1(6H)-yl)-2-(4-fluoro-3,5-dimethylphenyl)-4-methyl-2,4,6,7-tetrahydro-5H-pyrazolo[4,3-c]pyridine-5-carboxylate and tert-butyl (4S)-3-(5-chloro-4-(4-fluoro-1-methyl-1H-indazol-5-yl)-6-oxopyridazin-1(6H)-yl)-2-(4-fluoro-3,5-dimethylphenyl)-4-methyl-2,4,6,7-tetrahydro-5H-pyrazolo[4,3-c]pyridine-5-carboxylate

To a N₂ flushed mixture of tert-butyl (4S)-3-(4,5-dichloro-6-oxopyridazin-1(6H)-yl)-2-(4-fluoro-3,5-dimethylphenyl)-4-methyl-2,4,6,7-tetrahydro-5H-pyrazolo[4,3-c]pyridine-5-carboxylate (420 mg, 804 mol) and tert-butyl (4S)-3-(4,5-dichloro-6-oxopyridazin-1(6H)-yl)-2-(4-fluoro-3,5-dimethylphenyl)-4-methyl-2,4,6,7-tetrahydro-5H-pyrazolo[4,3-c]pyridine-5-carboxylate (420 mg, 1 Eq, 804 mol) in toluene (4 mL) was added Pd(PPh₃)₄(27.9 mg, 24.1 mol) and a 2M aqueous solution of Na₂CO₃ (1.21 mL, 2.41 mmol). The reaction mixture was heated to 120° C. and stirred at this temperature for 2 hours under a N₂ atmosphere. After cooling to 25° C., the reaction mixture was diluted with water (7 mL), and extracted with EtOAc (3×5 mL). Then organic layers were combined, dried over Na₂SO₄, filtered, and concentrated under reduced pressure to provide the crude residue which was purified by silica gel chromatography (Pet. Ether/EtOAc: 1/0 to 66/33) to provide a mixture of stereoisomers. The two stereoisomers were separated by chiral SFC (column name: Daicel Chiralcel OD ethanol (0.1% NH₃H₂O/isocratic) to afford the title compound. MS (ESI) m/z 636.3 [M+H]+.

### Step 4: tert-butyl (S)-3-(3-(4-fluoro-1-methyl-1H-indazol-5-yl)-2-oxopyridin-1(2H)-yl)-2-(4-fluoro-3,5-dimethylphenyl)-4-methyl-2,4,6,7-tetrahydro-5H-pyrazolo[4,3-c]pyridine-5-carboxylate

To a solution of tert-butyl (4S)-3-(4-chloro-5-(4-fluoro-1-methyl-1H-indazol-5-yl)-6-oxopyridazin-1(6H)-yl)-2-(4-fluoro-3,5-dimethylphenyl)-4-methyl-2,4,6,7-tetrahydro-5H-pyrazolo[4,3-c]pyridine-5-carboxylate (70 mg, 0.11 mmol) in EtOAc (5 mL) was added potassium carbonate (30 mg, 0.22 mmol) and palladium (5% on carbon) (47 mg, 22 μmol). The reaction mixture was agitated in a hydrogen atmosphere (15 psi) for 24 hours at 20° C. The reaction mixture was filtered and concentrated under reduced pressure to provide the title compound which was used in the next step without further purification. MS (ESI) m/z 602.5 [M+H]+.

### Step 5: 5-chloro-4-(4-fluoro-1-methyl-1H-indazol-5-yl)-2-((4S)-2-(4-fluoro-3,5-dimethylphenyl)-4-methyl-4,5,6,7-tetrahydro-2H-pyrazolo[4,3-c]pyridin-3-yl)pyridazin-3(2H)-one

These steps were performed in a similar fashion as described for example 1, step 4 to provide the title compound. MS (ESI) m/z 502.5 [M+H]+.

### Step 6: 3-((1S,2S)-1-(5-(2,2-dimethyltetrahydro-2H-pyran-4-yl)-2-((4S)-3-(5-(4-fluoro-1-methyl-1H-indazol-5-yl)-6-oxopyridazin-1(6H)-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one

These steps were performed in a similar fashion as described for example 1, step 5 to provide the title compound. MS (ESI) m/z: 895.3 [M+H]+. 1H NMR (400 MHz, acetonitrile-d3) δ ppm 11.31-11.55 (m, 1H), 8.10 (s, 1H), 7.79-8.05 (m, 1H), 7.44-7.59 (m, 3H), 7.23-7.43 (m, 2H), 6.92-7.09 (m, 2H), 6.71-6.88 (m, 1H), 5.69 (m, 0.6 H), 5.10-5.24 (m, 0.4 H), 4.69-4.82 (m, 0.4 H), 4.41 (m, 0.6 H), 4.00-4.08 (m, 3H), 3.53-3.82 (m, 3H), 3.11-3.30 (m, 1 H), 2.88-3.10 (m, 2H), 2.16-2.27 (m, 6H), 1.75-1.82 (m, 1H), 1.60-1.74 (m, 4H), 1.50-1.59 (m, 3H), 1.46 (m, 2H), 1.28 (m, 4H), 1.09-1.21 (m, 5H), 0.95 (m, 1H).

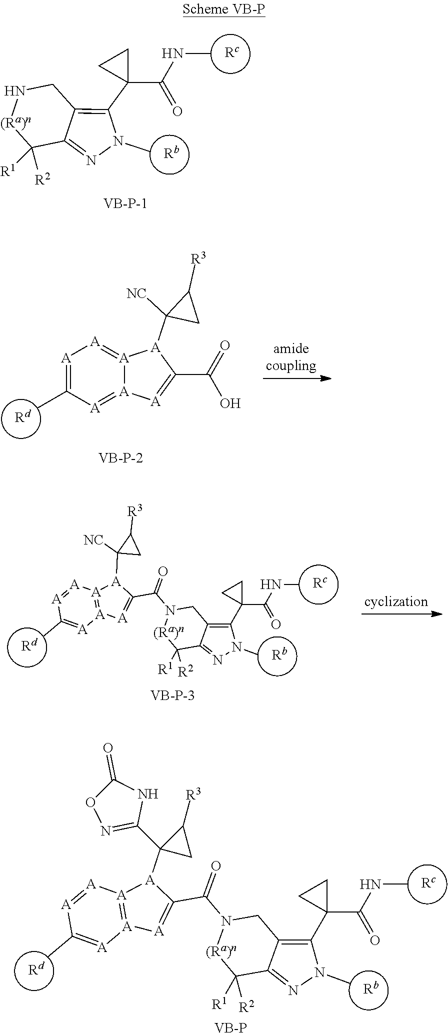

Compounds of formula VB—P are prepared from VB-P-1 through coupling with carboxylic acid VB—P-2 to provide VB—P-3 which can undergo a hydroxyl amine/CDI cyclization to provide compounds of formula VB—P.

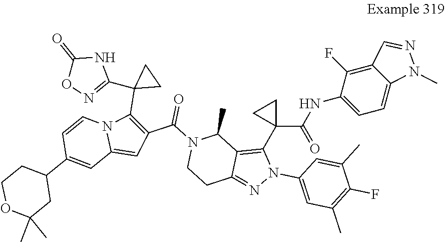 [SMILES: Cc1cc(-n2nc3c(c2C2(C(=O)Nc4ccc5c(cnn5C)c4F)CC2)[C@H](C)N(C(=O)c2cc4cc(C5CCOC(C)(C)C5)ccn4c2C2(c4noc(=O)[nH]4)CC2)CC3)cc(C)c1F]

### 1-((4S)-5-(7-(2,2-dimethyltetrahydro-2H-pyran-4-yl)-3-(1-(5-oxo-4,5-dihydro-1,2,4-oxadiazol-3-yl)cyclopropyl)indolizine-2-carbonyl)-2-(4-fluoro-3,5-dimethylphenyl)-4-methyl-4,5,6,7-tetrahydro-2H-pyrazolo[4,3-c]pyridin-3-yl)-N-(4-fluoro-1-methyl-1H-indazol-5-yl)cyclopropane-1-carboxamide

### Step 1: 1-((4S)-5-(7-(2,2-dimethyltetrahydro-2H-pyran-4-yl)-3-(1-(5-oxo-4,5-dihydro-1,2,4-oxadiazol-3-yl)cyclopropyl)indolizine-2-carbonyl)-2-(4-fluoro-3,5-dimethylphenyl)-4-methyl-4,5,6,7-tetrahydro-2H-pyrazolo[4,3-c]pyridin-3-yl)-N-(4-fluoro-1-methyl-1H-indazol-5-yl)cyclopropane-1-carboxamide

To a solution of 1-((4S)-5-(3-(1-cyanocyclopropyl)-7-(2,2-dimethyltetrahydro-2H-pyran-4-yl)indolizine-2-carbonyl)-2-(4-fluoro-3,5-dimethylphenyl)-4-methyl-4,5,6,7-tetrahydro-2H-pyrazolo[4,3-c]pyridin-3-yl)-N-(4-fluoro-1-methyl-1H-indazol-5-yl)cyclopropane-1-carboxamide (78 mg, 96 mol) in DMSO (0.18 mL) was added hydroxylamine (49 mg, 45 L, 50% wt, 20 eq, 0.74 mmol). The reaction mixture was then stirred at 60° C. for 1 hour. The mixture was cooled down to 25° C., diluted with EtOAc (20 mL) and washed with brine (2×10 mL). The organic layer was then dried over sodium sulfate, filtered, and concentrated under reduced pressure. The crude residue was dissolved in DMSO (0.18 mL), treated with DBU (14 L, 92 mol) and CDI (12 mg, 74 mol), and stirred for 1 hour at 50° C. The crude reaction mixture was purified via C18 reverse phase column chromatography (20-100% MeCN/water+0.1% FA modifier) to afford the title compound as the formic acid salt. ¹H NMR (500 MHz, CD₃CN) δ 8.18 (dd, J=30.4, 7.3 Hz, 1H), 8.05 (d, J=6.3 Hz, 1H), 7.97 (d, J=31.1 Hz, 1H), 7.43-7.16 (m, 4H), 6.75 (dd, J=7.4, 1.7 Hz, 1H), 6.53 (s, 1H), 5.87 (s, 1H), 4.94 (s, 1H), 4.29 (s, 1H), 4.05 (d, J=8.5 Hz, 3H), 3.84-3.70 (m, 2H), 3.50 (d, J=73.0 Hz, 1H), 3.17-2.70 (m, 3H), 2.34 (dd, J=11.5, 1.6 Hz, 7H), 1.77 (dd, J=19.1, 5.1 Hz, 5H), 1.70-1.43 (m, 5H), 1.37-1.09 (m, 8H).MS (ESI) m/z 870.4 [M+H]+.

The following examples in Table 28 were prepared according to scheme VB—P using the procedures outlined in the synthesis of example 319 with corresponding intermediates.

TABLE 28

| | | | MS |
|---|---|---|---|
| Ex | Structure | Name | (M+1) |
| 320 Isomer A (ChiralP ak IA, CO2: MeOH + 0.01% NH₄OH) | 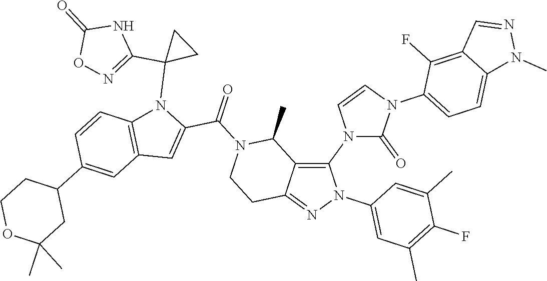 [SMILES: Cc1cc(-n2nc3c(c2-n2ccn(-c4ccc5c(cnn5C)c4F)c2=O)[C@H](C)N(C(=O)c2cc4cc(C5CCOC(C)(C)C5)ccc4n2C2(c4noc(=O)[nH]4)CC2)CC3)cc(C)c1F] | 3-(1-(7-(2,2- dimethyltetrahydro-2H- pyran-4-yl)-2-((S)-3-((R or S)-1-(4-fluoro-1-methyl-1H- indazol-5-yl)-2- oxopyrrolidin-3-yl)-2-(4- fluoro-3,5-dimethylphenyl)- 4-methyl-4,5,6,7-tetrahydro- 2H-pyrazolo[4,3-c]pyridine- 5-carbonyl)indolizin-3- yl)cyclopropyl)-1,2,4- oxadiazol-5(4H)-one | 870.4 |
| 321 Isomer B (ChiralP ak IA, CO2: MeOH + 0.01% NH₄OH) | 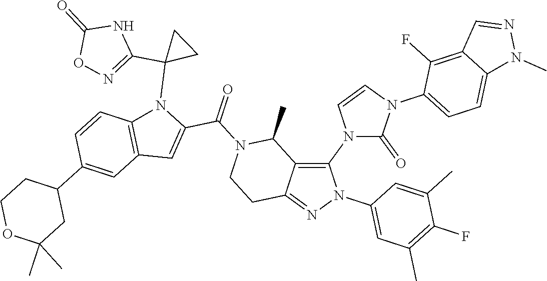 [SMILES: Cc1cc(-n2nc3c(c2-n2ccn(-c4ccc5c(cnn5C)c4F)c2=O)[C@H](C)N(C(=O)c2cc4cc(C5CCOC(C)(C)C5)ccc4n2C2(c4noc(=O)[nH]4)CC2)CC3)cc(C)c1F] | 3-(1-(7-(2,2- dimethyltetrahydro-2H- pyran-4-yl)-2-((S)-3-((R or S)-1-(4-fluoro-1-methyl-1H- indazol-5-yl)-2- oxopyrrolidin-3-yl)-2-(4- fluoro-3,5-dimethylphenyl)- 4-methyl-4,5,6,7-tetrahydro- 2H-pyrazolo[4,3-c]pyridine- 5-carbonyl)indolizin-3- yl)cyclopropyl)-1,2,4- oxadiazol-5(4H)-one | 870.4 |

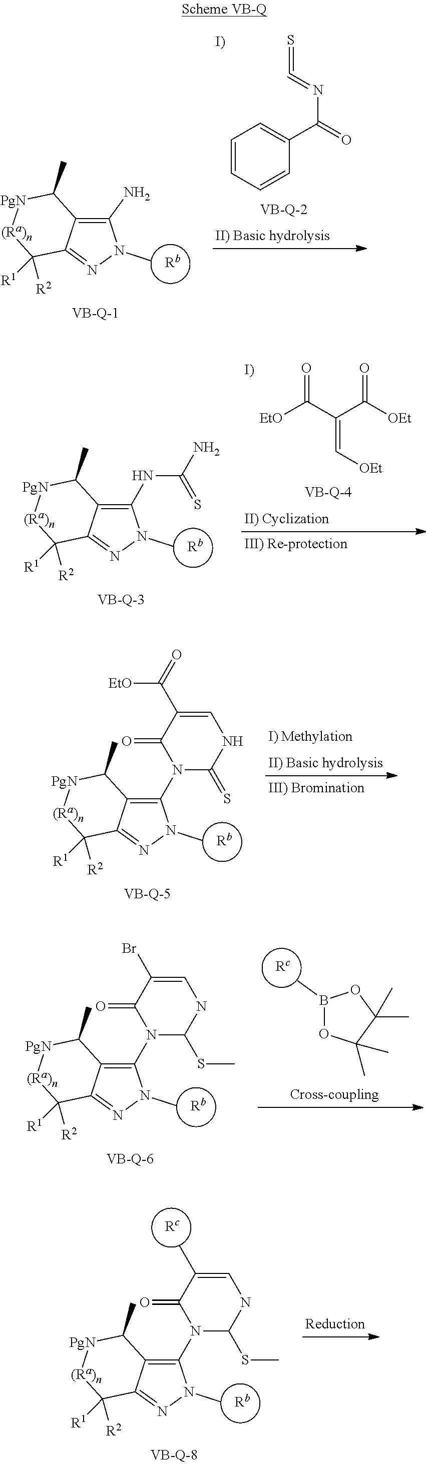

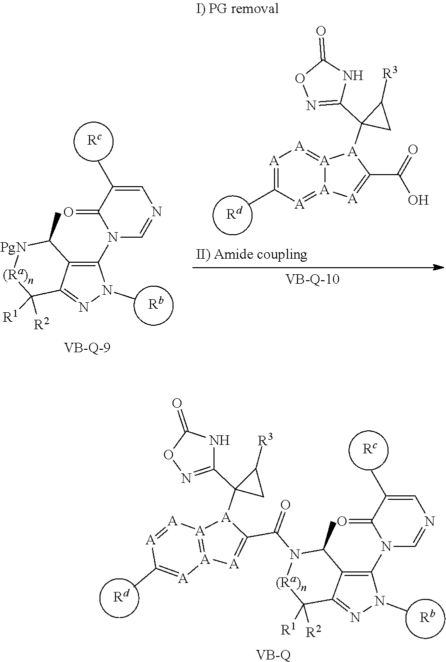

Compounds of formula VB-Q are prepared from VB-Q-1 by reacting with reagent VB-Q-2 which upon basic hydrolysis provides VB-Q-3. Reaction with VB-Q-4 followed by acid-mediated cyclization and re-protection of the amine provides compounds of the form VB-Q-5. Methylation of the sulfur unit using methyl iodide followed by basic hydrolysis of the ester and finally bromination provides compounds of the form VB-Q-6. Metal-mediated cross-coupling with various boronic acids or esters can provide compounds of the form VB-Q-8. Reduction of the sulfur unit under Pd/C conditions provides VB-Q-9 which upon protecting group removal (e.g., acidic conditions) and subsequent coupling with carboxylic acid VB-Q-10 provides compounds of formula VB-Q.

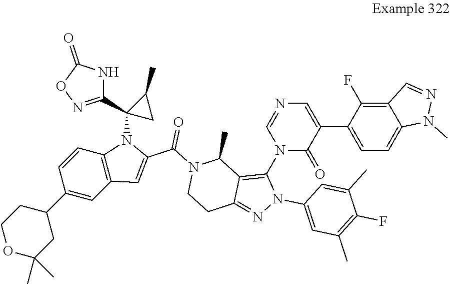 [SMILES: Cc1cc(-n2nc3c(c2-n2cncc(-c4ccc5c(cnn5C)c4F)c2=O)[C@H](C)N(C(=O)c2cc4cc(C5CCOC(C)(C)C5)ccc4n2[C@@]2(c4noc(=O)[nH]4)C[C@@H]2C)CC3)cc(C)c1F]

### 3-((1S,2S)-1-(5-((S)-2,2-dimethyltetrahydro-2H-pyran-4-yl)-2-((4S)-3-(5-(4-fluoro-1-methyl-1H-indazol-5-yl)-6-oxopyrimidin-1(6H)-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one

### Step 1: tert-butyl (S)-3-(3-benzoylthioureido)-2-(4-fluoro-3,5-dimethylphenyl)-4-methyl-2,4,6,7-tetrahydro-5H-pyrazolo[4,3-c]pyridine-5-carboxylate

To a solution of tert-butyl (S)-3-amino-2-(4-fluoro-3,5-dimethylphenyl)-4-methyl-2,4,6,7-tetrahydro-5H-pyrazolo[4,3-c]pyridine-5-carboxylate (1000 mg, 2.67 mmol) in acetone (15 mL) was added benzoyl isothiocyanate (480 mg, 2.94 mmol) at 25° C. The reaction mixture was stirred at 60° C. for 4 hours. The mixture was concentrated under reduced pressure to provide the crude title compound which was used in the next step without further purification. MS (ESI) m/z 538.4 [M+H]+

### Step 2: tert-butyl (S)-2-(4-fluoro-3,5-dimethylphenyl)-4-methyl-3-thioureido-2,4,6,7-tetrahydro-5H-pyrazolo[4,3-c]pyridine-5-carboxylate

To a solution of tert-butyl (S)-3-(3-benzoylthioureido)-2-(4-fluoro-3,5-dimethylphenyl)-4-methyl-2,4,6,7-tetrahydro-5H-pyrazolo[4,3-c]pyridine-5-carboxylate (5.0 g, 9.3 mmol) in MeOH (60 mL) was added K₂CO₃ (2.4 g, 18 mmol) at 20° C. Then the reaction mixture was stirred at 20° C. for 16 hours. The mixture was concentrated under reduced pressure to provide the crude product, which was purified by silica gel chromatography (Pet. Ether/EtOAc: 91/9) to provide the title compound. MS (ESI) m/z 434.3 [M+H]+

### Step 3: diethyl (S)-2-((3-(5-(tert-butoxycarbonyl)-2-(4-fluoro-3,5-dimethylphenyl)-4-methyl-4,5,6,7-tetrahydro-2H-pyrazolo[4,3-c]pyridin-3-yl)thioureido)methylene)malonate

To a solution of tert-butyl (S)-2-(4-fluoro-3,5-dimethylphenyl)-4-methyl-3-thioureido-2,4,6,7-tetrahydro-5H-pyrazolo[4,3-c]pyridine-5-carboxylate (2.9 g, 6.7 mmol), diethyl 2-(ethoxymethylene)malonate (1.4 g, 6.7 mmol) in anhydrous DMF (20 mL) was added K₂CO₃ (1.8 g, 13 mmol) and pyridine (2.2 mL, 27 mmol). The resultant mixture was stirred at 120° C. for 4 hours. The reaction was quenched with water (50 mL) and extracted with EtOAc (3×60 mL). The organic layers were combined, washed with brine (40 mL), dried over Na₂SO₄, and filtered. The crude solution was concentrated under reduced pressure and purified by silica gel column chromatography (Pet. ether/EtOAc: 2/1) to provide the title compound. MS (ESI) m/z 604.2 [M+H]+

### Step 4: ethyl 1-((4S)-2-(4-fluoro-3,5-dimethylphenyl)-4-methyl-4,5,6,7-tetrahydro-2H-pyrazolo[4,3-c]pyridin-3-yl)-4-oxo-2-thioxo-1,2,3,4-tetrahydropyrimidine-5-carboxylate

To a solution of diethyl (S)-2-((3-(5-(tert-butoxycarbonyl)-2-(4-fluoro-3,5-dimethylphenyl)-4-methyl-4,5,6,7-tetrahydro-2H-pyrazolo[4,3-c]pyridin-3-yl)thioureido)methylene)malonate (600 mg, 994 mol) in ethanol (6 mL) was added 12 M aq. HCl (1 mL, 0.01 mol). The resultant mixture was stirred at 60° C. for 36 hours. The reaction was concentrated under reduced pressure to provide the title compound. The crude material was used directly in the next step without further purification. MS (ESI) m/z 458.1 [M+H]+

### Step 5: tert-butyl (4S)-3-(5-(ethoxycarbonyl)-6-oxo-2-thioxo-3,6-dihydropyrimidin-1(2H)-yl)-2-(4-fluoro-3,5-dimethylphenyl)-4-methyl-2,4,6,7-tetrahydro-5H-pyrazolo[4,3-c]pyridine-5-carboxylate

To a solution of ethyl 3-((4S)-2-(4-fluoro-3,5-dimethylphenyl)-4-methyl-4,5,6,7-tetrahydro-2H-pyrazolo[4,3-c]pyridin-3-yl)-4-oxo-2-thioxo-1,2,3,4-tetrahydropyrimidine-5-carboxylate (450 mg, 984 mol) and triethylamine (548 μL, 3.93 mmol) in DCM (6 mL) was added Boc₂O (322 mg, 1.48 mmol). The resultant mixture was stirred at 25° C. for 16 hours. The reaction mixture was poured into water (10 mL), and extracted with EtOAc (3×15 mL), washed with brine (2×40 mL), dried over Na₂SO₄, filtered, and concentrated under reduced pressure to provide a crude residue, which was purified by silica gel chromatography (Pet.ether/EtOAc: 1/1) to provide the title compound. MS (ESI) m/z 558.2 [M+H]+.

### Step 6: tert-butyl (4S)-3-(5-(ethoxycarbonyl)-2-(methylthio)-6-oxopyrimidin-1(6H)-yl)-2-(4-fluoro-3,5-dimethylphenyl)-4-methyl-2,4,6,7-tetrahydro-5H-pyrazolo[4,3-c]pyridine-5-carboxylate

To a solution of tert-butyl (4S)-3-(5-(ethoxycarbonyl)-6-oxo-2-thioxo-3,6-dihydropyrimidin-1(2H)-yl)-2-(4-fluoro-3,5-dimethylphenyl)-4-methyl-2,4,6,7-tetrahydro-5H-pyrazolo[4,3-c]pyridine-5-carboxylate (122 mg, 218 mol) and Na₂CO₃ (69.4 mg, 655 mol) in DMF (2 mL) was added methyl iodide (42 μL, 655 mol). The resultant mixture was stirred at 20° C. for 16 hours. The reaction mixture was poured into water (3 mL), and extracted with EtOAc (3×5 mL), washed with brine (2×10 mL), dried over Na₂SO₄, filtered, and concentrated under reduced pressure to provide the crude residue, which was purified by silica gel chromatography (Pet.ether/EtOAc: 1/1) to provide the title compound. MS (ESI) m/z 572.2 [M+H]+.

### Step 7: 1-((4S)-5-(tert-butoxycarbonyl)-2-(4-fluoro-3,5-dimethylphenyl)-4-methyl-4,5,6,7-tetrahydro-2H-pyrazolo[4,3-c]pyridin-3-yl)-2-(methylthio)-6-oxo-1,6-dihydropyrimidine-5-carboxylic acid

To a solution of tert-butyl (4S)-3-(5-(ethoxycarbonyl)-2-(methylthio)-6-oxopyrimidin-1(6H)-yl)-2-(4-fluoro-3,5-dimethylphenyl)-4-methyl-2,4,6,7-tetrahydro-5H-pyrazolo[4,3-c]pyridine-5-carboxylate (300 mg, 525 mol) in THF (2 mL) and H₂O (2 mL) was added lithium hydroxide hydrate (66 mg, 1.6 mmol) and the mixture is stirred for 60 min at 15° C. The reaction mixture was quenched by 1 M aq. HCl (2 mL), and extracted with EtOAc (3×2 mL). The organic layers were combined, dried over Na₂SO₄, filtered, and concentrated under reduced pressure to provide the crude title compound which was used directly in the next step without further purification. MS (ESI) m/z 544.2 [M+H]+.

### Step 8: tert-butyl (4S)-3-(5-bromo-2-(methylthio)-6-oxopyrimidin-1(6H)-yl)-2-(4-fluoro-3,5-dimethylphenyl)-4-methyl-2,4,6,7-tetrahydro-5H-pyrazolo[4,3-c]pyridine-5-carboxylate

To a solution of 1-((4S)-5-(tert-butoxycarbonyl)-2-(4-fluoro-3,5-dimethylphenyl)-4-methyl-4,5,6,7-tetrahydro-2H-pyrazolo[4,3-c]pyridin-3-yl)-2-(methylthio)-6-oxo-1,6-dihydropyrimidine-5-carboxylic acid (262 mg, 482 mol) in MeCN (4 mL) was added potassium phosphate (716 mg, 3.37 mmol) and monopyridin-1-ium tribromide (482 mg, 1.51 mmol). The resultant reaction mixture was stirred at 80° C. for 16 hours. The reaction solution was filtered and concentrated under reduced pressure to provide the crude residue which was purified by prep-TLC on silica gel (EtOAc/Hexane: 1/1) to provide the title compound. MS (ESI) m/z 579.9 [M+H]+.

### Step 9: tert-butyl (4S)-3-(5-(4-fluoro-1-methyl-1H-indazol-5-yl)-2-(methylthio)-6-oxopyrimidin-1(6H)-yl)-2-(4-fluoro-3,5-dimethylphenyl)-4-methyl-2,4,6,7-tetrahydro-5H-pyrazolo[4,3-c]pyridine-5-carboxylate

To a mixture of tert-butyl (4S)-3-(5-bromo-2-(methylthio)-6-oxopyrimidin-1(6H)-yl)-2-(4-fluoro-3,5-dimethylphenyl)-4-methyl-2,4,6,7-tetrahydro-5H-pyrazolo[4,3-c]pyridine-5-carboxylate (67 mg, 0.12 mmol), 4-fluoro-1-methyl-5-(4,4,5,5-tetramethyl-1,3,2-dioxaborolan-2-yl)-1H-inda-le (48 mg, 0.17 mmol) and potassium phosphate (74 mg, 0.35 mmol) in 1,4-dioxane (2 mL) and water (0.4 mL) was added 1,1′-bis(diphenylphosphino)ferrocene-palladium(II) dichloride (8.5 mg, 12 mol). The resultant mixture was stirred at 80° C. for 1 hour under a N₂ atmosphere. The reaction mixture was filtered and extracted with EtOAc (3×3 mL) and H₂O (3 mL). The combined organic layers were washed with brine (5 mL), dried over Na₂SO₄, filtered, and concentrated under reduced pressure. The crude residue was purified by prep-TLC on silica gel (hexane/EtOAc: 3/1) to provide the title compound. MS (ESI) m/z 648.3 [M+H]+.

### Step 10: tert-butyl (4S)-3-(5-(4-fluoro-1-methyl-1H-indazol-5-yl)-6-oxopyrimidin-1(6H)-yl)-2-(4-fluoro-3,5-dimethylphenyl)-4-methyl-2,4,6,7-tetrahydro-5H-pyrazolo[4,3-c]pyridine-5-carboxylate

To a solution of tert-butyl (4S)-3-(5-(4-fluoro-1-methyl-1H-indazol-5-yl)-2-(methylthio)-6-oxopyrimidin-1(6H)-yl)-2-(4-fluoro-3,5-dimethylphenyl)-4-methyl-2,4,6,7-tetrahydro-5H-pyrazolo[4,3-c]pyridine-5-carboxylate (50 mg, 77 μmol) in MeOH (0.5 mL) was added Pd/C (8.2 mg, 10% wt, 7.7 mol) and triethylsilane (90 mg, 772 μmol). The resultant mixture was stirred at 30° C. for 16 hours. The reaction mixture was filtered and concentrated under reduced pressure to provide the crude residue that was purified by reverse phase column chromatography (60-90% MeCN/water+0.1% TFA modifier) to provide the title compound. MS (ESI) m/z 602.3 [M+H]+.

### Step 11: 5-(4-fluoro-1-methyl-1H-indazol-5-yl)-3-((4S)-2-(4-fluoro-3,5-dimethylphenyl)-4-methyl-4,5,6,7-tetrahydro-2H-pyrazolo[4,3-c]pyridin-3-yl)pyrimidin-4(3H)-one

A solution of tert-butyl (4S)-3-(5-(4-fluoro-1-methyl-1H-indazol-5-yl)-6-oxopyrimidin-1(6H)-yl)-2-(4-fluoro-3,5-dimethylphenyl)-4-methyl-2,4,6,7-tetrahydro-5H-pyrazolo[4,3-c]pyridine-5-carboxylate (20 mg, 33 mol) in 2 M HCl/dioxane (2 mL) was stirred at 30° C. for 2 hours. The reaction mixture was concentrated under reduced pressure to provide the crude title compound which was used directly in the next step without further purification. MS (ESI) m/z 502.2 [M+H]+.

### Step 12: 3-((1S,2S)-1-(5-((S)-2,2-dimethyltetrahydro-2H-pyran-4-yl)-2-((4S)-3-(5-(4-fluoro-1-methyl-1H-indazol-5-yl)-6-oxopyrimidin-1(6H)-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one

These steps were performed in a similar fashion as described for example 1 to provide the title compound. MS (ESI) m/z 895.4 [M+H]+. ¹H NMR (400 MHz, CD₃CN) δ ppm 11.22-11.57 (m, 1H), 8.20 (s, 0.3 H), 8.03-8.13 (m, 1.7 H), 7.82-8.02 (m, 1H), 7.56 (s, 2H), 7.38-7.47 (m, 1H), 7.37 (s, 0.5 H), 7.29 (br d, J=8.58 Hz, 1.2 H), 7.07 (t, J=6.79 Hz, 1.3 H), 6.94-7.04 (m, 1H), 6.74-6.88 (m, 1H), 5.63-5.84 (m, 0.6 H), 5.13-5.42 (m, 0.4 H), 4.71-4.89 (m, 0.4 H), 4.39-4.51 (m, 0.6 H), 4.00-4.10 (m, 3H), 3.58-3.81 (m, 3H), 2.87-3.24 (m, 3H), 2.21 (br s, 6H), 1.75-1.81 (m, 1H), 1.68-1.74 (m, 2H), 1.60-1.68 (m, 2H), 1.57 (br d, J=6.79 Hz, 2H), 1.47 (dd, J=18.12, 6.79 Hz, 2H), 1.25-1.31 (m, 4H), 1.16-1.21 (m, 3H), 1.14 (dd, J=5.96, 2.62 Hz, 2H), 1.08 (d, J=6.56 Hz, 0.5 H), 0.93 (d, J=6.32 Hz, 0.5 H).

The following examples in Table 29 were prepared according to scheme VB-Q using the procedures outlined in the synthesis of example 322.

TABLE 29

| | | | MS |
|---|---|---|---|
| Ex. | Structure | Name | (M+1) |
| 323 | 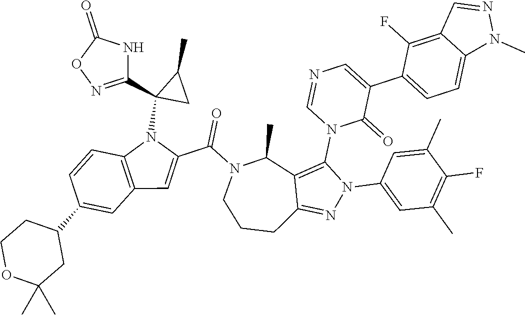 | 3-[(1S,2S)-1-[5-[(4S)-2,2- dimethyltetrahydropyran-4- yl]-2-[(4S)-2-(4-fluoro-3,5- dimethyl-phenyl)-3-[5-(4- fluoro-1-methyl-indazol-5- yl)-6-oxo-pyrimidin-1-yl]- 4-methyl-4,6,7,8- tetrahydropyrazolo[4,3- c]azepine-5-carbonyl]indol- 1-yl]-2-methyl- cyclopropyl]-4H-1,2,4- oxadiazol-5-one | 909.4 |

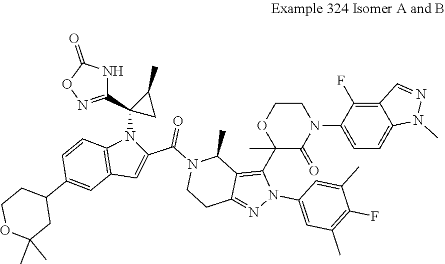 [SMILES: Cc1cc(-n2nc3c(c2C2(C)OCCN(c4ccc5c(cnn5C)c4F)C2=O)[C@H](C)N(C(=O)c2cc4cc(C5CCOC(C)(C)C5)ccc4n2[C@@]2(c4noc(=O)[nH]4)C[C@@H]2C)CC3)cc(C)c1F]

### (R)-2-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)-4-(4-fluoro-1-methyl-1H-indazol-5-yl)-2-methylmorpholin-3-one and (S)-2-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)-4-(4-fluoro-1-methyl-1H-indazol-5-yl)-2-methylmorpholin-3-one

### Step 1: tert-butyl (4S)-3-(1-ethoxy-2-hydroxy-1-oxopropan-2-yl)-2-(4-fluoro-3,5-dimethylphenyl)-4-methyl-2,4,6,7-tetrahydro-5H-pyrazolo[4,3-c]pyridine-5-carboxylate

To a solution of tert-butyl (S)-3-bromo-2-(4-fluoro-3,5-dimethylphenyl)-4-methyl-2,4,6,7-tetrahydro-5H-pyrazolo[4,3-c]pyridine-5-carboxylate (2.0 g, 4.6 mmol) in THF (20 mL) was added n-butyllithium (3.65 mL, 2.5 M in hexanes, 9.13 mmol) at −78° C. under a N₂ atmosphere. The resultant mixture was stirred at −78° C. for 1 hour at which time ethyl 2-oxopropanoate (1.06 g, 9.13 mmol) was added at −78° C. The resultant reaction mixture was stirred for 2 hours at −78° C. The mixture was quenched with aq. NH₄Cl solution (20 mL) at 0° C. and extracted with EtOAc (4×50 mL). The combined organic extracts were washed with brine (50 mL), dried over anhydrous Na₂SO₄, filtered and concentrated under reduced pressure. The crude residue was purified by reverse phase column chromatography (61-81% MeCN/water+0.05% TFA modifier) to provide the title compound. MS (ESI) m/z 476.3 [M+H]+.

### Step 2: 2-((S)-5-(tert-butoxycarbonyl)-2-(4-fluoro-3,5-dimethylphenyl)-4-methyl-4,5,6,7-tetrahydro-2H-pyrazolo[4,3-c]pyridin-3-yl)-2-hydroxypropanoic acid

To a solution of tert-butyl (4S)-3-(1-ethoxy-2-hydroxy-1-oxopropan-2-yl)-2-(4-fluoro-3,5-dimethylphenyl)-4-methyl-2,4,6,7-tetrahydro-5H-pyrazolo[4,3-c]pyridine-5-carboxylate (300 mg, 631 mol) in THF (3 mL) was added potassium trimethylsilanolate (162 mg, 1.26 mmol) at 25° C. The resultant mixture was stirred at 25° C. for 18 hours. The reaction mixture was concentrated to provide the crude title compound which was used directly in the next reaction without further purification. MS (ESI) m/z 448.1 [M+H]+.

### Step 3: tert-butyl (4S)-3-(1-((4-fluoro-1-methyl-1H-indazol-5-yl)amino)-2-hydroxy-1-oxopropan-2-yl)-2-(4-fluoro-3,5-dimethylphenyl)-4-methyl-2,4,6,7-tetrahydro-5H-pyrazolo[4,3-c]pyridine-5-carboxylate

To a solution of potassium 2-((S)-5-(tert-butoxycarbonyl)-2-(4-fluoro-3,5-dimethylphenyl)-4-methyl-4,5,6,7-tetrahydro-2H-pyrazolo[4,3-c]pyridin-3-yl)-2-hydroxypropanoate (282 mg, 581 mol), 4-fluoro-1-methyl-1H-indazol-5-amine hydrochloride (234 mg, 1.16 mmol) in DMF (1 mL) was added DIPEA (405 μL, 2.32 mmol) and HATU (331 mg, 871 μmol) at 20° C. The resultant reaction mixture was stirred at 20° C. for 16 hours. The crude residue was purified by reverse phase column chromatography (42-72% MeCN/water+0.1% TFA modifier) to provide the title compound. MS (ESI) m/z 595.3 [M+H]+.

### Step 4: tert-butyl (S)-3-((R)-4-(4-fluoro-1-methyl-1H-indazol-5-yl)-2-methyl-3-oxomorpholin-2-yl)-2-(4-fluoro-3,5-dimethylphenyl)-4-methyl-2,4,6,7-tetrahydro-5H-pyrazolo[4,3-c]pyridine-5-carboxylate and tert-butyl (S)-3-((S)-4-(4-fluoro-1-methyl-1H-indazol-5-yl)-2-methyl-3-oxomorpholin-2-yl)-2-(4-fluoro-3,5-dimethylphenyl)-4-methyl-2,4,6,7-tetrahydro-5H-pyrazolo[4,3-c]pyridine-5-carboxylate

To a solution of tert-butyl (4S)-3-(1-((4-fluoro-1-methyl-1H-indazol-5-yl)amino)-2-hydroxy-1-oxopropan-2-yl)-2-(4-fluoro-3,5-dimethylphenyl)-4-methyl-2,4,6,7-tetrahydro-5H-pyrazolo[4,3-c]pyridine-5-carboxylate (100 mg, 168 mol) in DCM (1 mL) was added potassium hydroxide (23.6 mg, 420 mol) at 0° C. followed by diphenyl(vinyl)sulfonium trifluoromethanesulfonate (76.2 mg, 210 mol). The resultant mixture was warmed to 25° C. and stirred for 16 hours. The reaction mixture was concentrated under reduced pressure and purified by reverse phase column chromatography (60-90% MeCN/water+0.05% NH₃·H₂O+10 mM NH₄·HCO₃ modifier) to provide title compound as a mixture of stereoisomers. The stereoisomers were separated by chiral SFC (column name: Daicel Chiralpak IG ethanol/isocratic to afford the title compounds. Isomer A: MS (ESI) m/z 621.4 [M+H]+. Isomer B: MS (ESI) m/z 621.4 [M+H]+.

### Step 5: (R)-4-(4-fluoro-1-methyl-1H-indazol-5-yl)-2-((S)-2-(4-fluoro-3,5-dimethylphenyl)-4-methyl-4,5,6,7-tetrahydro-2H-pyrazolo[4,3-c]pyridin-3-yl)-2-methylmorpholin-3-one and (S)-4-(4-fluoro-1-methyl-1H-indazol-5-yl)-2-((S)-2-(4-fluoro-3,5-dimethylphenyl)-4-methyl-4,5,6,7-tetrahydro-2H-pyrazolo[4,3-c]pyridin-3-yl)-2-methylmorpholin-3-one

These steps were performed in a similar fashion as described for example 1, step 4 to provide the title compounds. Isomer A: MS (ESI) m/z 521.4 [M+H]+. Isomer B: MS (ESI) m/z 521.4 [M+H]+.

### Step 6: (R)-2-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)-4-(4-fluoro-1-methyl-1H-indazol-5-yl)-2-methylmorpholin-3-one and (S)-2-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)-4-(4-fluoro-1-methyl-1H-indazol-5-yl)-2-methylmorpholin-3-one

These steps were performed in a similar fashion as described for example 1, step 5 to provide the title compounds. Isomer A: MS (ESI) m/z 914.4 [M+H]+¹H NMR (400 MHz, CD₃CN) δ ppm 11.40-11.76 (m, 1H), 8.10 (s, 1H), 7.49-7.58 (m, 1H), 7.41-7.48 (m, 0.5 H), 7.24-7.40 (m, 2H), 7.17 (br d, J=6.32 Hz, 1H), 7.13 (br d, J=6.32 Hz, 1H), 7.04-7.11 (m, 1.5 H), 6.82 (s, 0.5 H), 6.69 (s, 0.5 H), 5.99-6.13 (m, 0.5 H), 5.69 (m, 0.6 H), 4.72 (m, 0.5 H), 4.36 (m, 0.5 H), 4.18-4.27 (m, 0.6 H), 4.04-4.16 (m, 3H), 3.92-4.00 (m, 1H), 3.84-3.91 (m, 0.6 H), 3.70-3.83 (m, 3H), 3.52-3.69 (m, 1H), 3.40-3.49 (m, 0.6 H), 2.80-3.16 (m, 3H), 2.77 (br t, J=12.28 Hz, 0.4 H), 2.32 (br d, J=11.68 Hz, 6H), 1.83-1.91 (m, 2H), 1.75-1.82 (m, 3H), 1.42-1.73 (m, 4H), 1.28-1.36 (m, 6H), 1.14-1.27 (m, 6H), 0.91 (d, J=5.84 Hz, 1H) Isomer B: MS (ESI) m/z 914.4 [M+H]+¹H NMR (400 MHz, CD₃CN) δ ppm 11.35-11.95 (m, 1H), 7.90-8.15 (m, 1H), 7.51-7.59 (m, 1.5 H), 7.42-7.50 (m, 1H), 7.26-7.40 (m, 1H), 7.14-7.26 (m, 2H), 7.11 (d, J=6.32 Hz, 0.5 H), 6.99 (m, 0.7 H), 6.81-6.90 (m, 1H), 6.63 (br t, J=7.81 Hz, 0.3 H), 6.05-6.07 (m, 0.7 H), 5.69 (d, J=6.56 Hz, 0.3 H), 4.75 (m, 0.3 H), 4.37 (m, 0.7 H), 4.11-4.20 (m, 2H), 4.07 (s, 3H), 3.89-3.97 (m, 1H), 3.74-3.81 (m, 2H), 3.61-3.70 (m, 1H), 3.41-3.60 (m, 1H), 2.89-3.23 (m, 4H), 2.26-2.38 (m, 6H), 1.79-1.86 (m, 2H), 1.67-1.78 (m, 3 H), 1.49-1.66 (m, 6H), 1.29-1.33 (m, 3H), 1.20-1.27 (m, 3H), 1.15-1.20 (m, 3H), 0.97 (d, J=5.96 Hz, 1H)

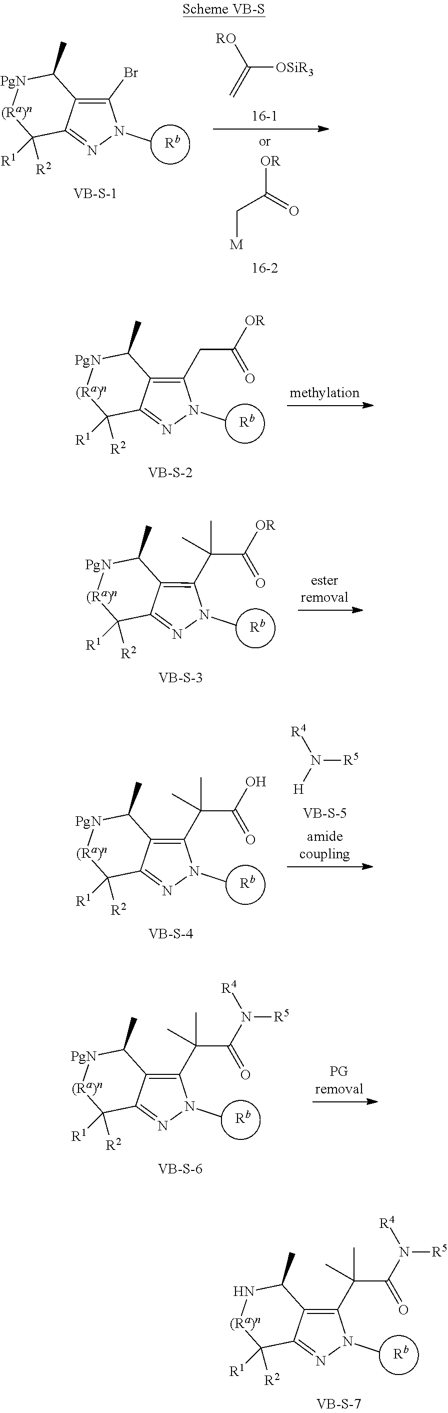

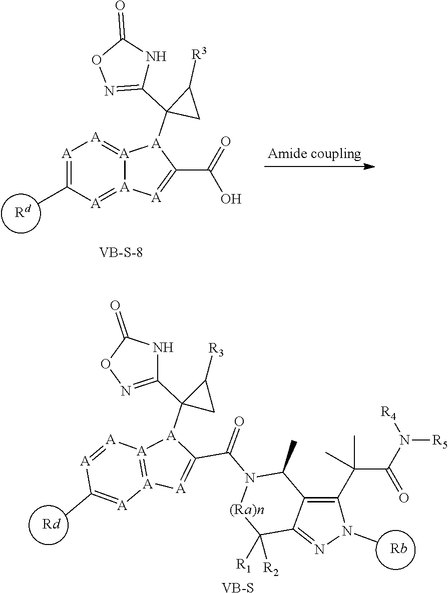

Compounds of formula VB—S are prepared from VB—S-1 by coupling with silyl enol ethers (16-1) or metal enolates such as zinc enolates (16-2, depicted arbitrarily as one possible tautomer) via transition metal catalyzed cross-coupling conditions (e.g., Pd—or Cu-catalysis) to provide VB—S-2. Methylation of the ester provides VB—S-3 which upon removal of the ester (e.g., basic aqueous conditions) furnishes carboxylic acid VB—S-4. Subsequently, VB—S-4 is coupled with amines VB—S-5 to provide amide VB—S-6. Removal of the protecting group (e.g., acidic conditions) of VB—S-6 provides amine VB—S-7 that is then coupled with carboxylic acid VB—S-8 to provide compounds of formula VB—S.

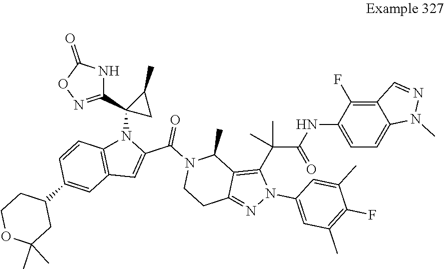

### 2-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)-N-(4-fluoro-1-methyl-1H-indazol-5-yl)-2-methylpropanamide

### Step 1: tert-butyl (S)-2-(4-fluoro-3,5-dimethylphenyl)-3-(2-methoxy-2-oxoethyl)-4-methyl-2,4,6,7-tetrahydro-5H-pyrazolo[4,3-c]pyridine-5-carboxylate

Step 1 was performed in a similar fashion as described for step 1 in example 78 to provide the title compound. MS (ESI) m/z 432.4 [M+H]+.

### Step 2: tert-butyl (S)-2-(4-fluoro-3,5-dimethylphenyl)-3-(1-methoxy-2-methyl-1-oxopropan-2-yl)-4-methyl-2,4,6,7-tetrahydro-5H-pyrazolo[4,3-c]pyridine-5-carboxylate

To a solution of tert-butyl (S)-2-(4-fluoro-3,5-dimethylphenyl)-3-(2-methoxy-2-oxoethyl)-4-methyl-2,4,6,7-tetrahydro-5H-pyrazolo[4,3-c]pyridine-5-carboxylate (340 mg, 788 mol) in THF (7 mL) was added sodium hydride (94.6 mg, 60% wt, 2.36 mmol) at 0° C., the reaction mixture was stirred at 0° C. for 30 min under a N₂ atmosphere. To the reaction mixture was added Mel (148 μL, 2.36 mmol) at 0° C. and the reaction mixture was warmed to 35° C. and stirred at this temperature for 16 hours under a N₂ atmosphere. The reaction mixture was quenched with aq. NH₄Cl (10 mL) and extracted with EtOAc (3×10 mL). The organic layers were combined, dried over Na₂SO₄, filtered, and concentrated under reduced pressure to provide the crude product which was purified by C18 reverse phase column chromatography (60-80% MeCN/water+0.1% TFA modifier) to provide the title compound. MS (ESI) m/z 460.8 [M+H]+.

### Step 3: (S)-2-(5-(tert-butoxycarbonyl)-2-(4-fluoro-3,5-dimethylphenyl)-4-methyl-4,5,6,7-tetrahydro-2H-pyrazolo[4,3-c]pyridin-3-yl)-2-methylpropanoic acid

To a solution of tert-butyl (S)-2-(4-fluoro-3,5-dimethylphenyl)-3-(1-methoxy-2-methyl-1-oxopropan-2-yl)-4-methyl-2,4,6,7-tetrahydro-5H-pyrazolo[4,3-c]pyridine-5-carboxylate (120 mg, 261 μmol) in THF (1 mL), H₂O (1 mL), and MeOH (0.5 mL) was added sodium hydroxide (104 mg, 2.61 mmol), the reaction mixture was stirred for 16 hours at 60° C. The reaction mixture was diluted with ethyl acetate (3 mL) and aqueous 1 M HCl (2 mL). The organic layer was separated, and the aqueous phase was extracted with ethyl acetate (3×3 mL). The organic layers were combined, washed with brine, dried over sodium sulfate, and concentrated to afford the crude title compound, which was used in the next step without further purification. MS (ESI) m/z 446.1 [M+H]+.

### Step 4: tert-butyl (S)-3-(1-((4-fluoro-1-methyl-1H-indazol-5-yl)amino)-2-methyl-1-oxopropan-2-yl)-2-(4-fluoro-3,5-dimethylphenyl)-4-methyl-2,4,6,7-tetrahydro-5H-pyrazolo[4,3-c]pyridine-5-carboxylate

To a solution of (S)-2-(5-(tert-butoxycarbonyl)-2-(4-fluoro-3,5-dimethylphenyl)-4-methyl-4,5,6,7-tetrahydro-2H-pyrazolo[4,3-c]pyridin-3-yl)-2-methylpropanoic acid (50 mg, 0.11 mmol), 4-fluoro-1-methyl-1H-indazol-5-amine (37 mg, 0.22 mmol), and 1-methyl-1H-imidazole (55 mg, 0.67 mmol) in MeCN (1 mL) was added TCFH (38 mg, 0.13 mmol), and the mixture was stirred at 15° C. for 16 hours. The reaction mixture was concentrated under reduced pressure to provide crude product, which was purified by flash silica gel chromatography (Biotage; 4 g Agela Silica Flash Column, Eluent of 0-35% EtOAc/Pet.ether gradient @30 mL/min) to provide the title compound. MS (ESI) m/z: 593.3 [M+H⁺].

### Step 5: (S)—N-(4-fluoro-1-methyl-1H-indazol-5-yl)-2-(2-(4-fluoro-3,5-dimethylphenyl)-4-methyl-4,5,6,7-tetrahydro-2H-pyrazolo[4,3-c]pyridin-3-yl)-2-methylpropanamide

These steps were performed in a similar fashion as described for example 1, step 4 to provide the crude title compound. MS (ESI) m/z 493.3 [M+H]+.

### Step 6: 2-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)-N-(4-fluoro-1-methyl-1H-indazol-5-yl)-2-methylpropanamide

These steps were performed in a similar fashion as described for example 1, step 5 to provide the title compound. MS (ESI) m/z 886.4 [M+H]+. ¹H NMR (400 MHz, CD₃CN) δ ppm 11.43-11.65 (m, 1H), 7.92 (s, 1H), 7.83 (s, 0.6 H), 7.67-7.72 (m, 0.4 H), 7.59 (s, 2.4 H), 7.25-7.37 (m, 2H), 7.03-7.12 (m, 2H), 6.82-6.91 (m, 1H), 6.72-6.77 (m, 0.3 H), 6.28-6.34 (m, 0.3 H), 6.03 (q, J=6.76 Hz, 0.7 H), 5.51-5.63 (in, 0.3 H), 4.65-4.79 (in, 0.3 H), 4.26-4.46 (in, 0.7 H), 3.92-4.06 (m, 3H), 3.52-3.83 (m, 3H), 2.82-3.16 (m, 3H), 2.21 (d, J=1.55 Hz, 6H), 1.83-1.87 (m, 1H), 1.77-1.82 (m, 1H), 1.68-1.77 (m, 3H), 1.60-1.68 (m, 5H), 1.48-1.60 (m, 4H), 1.28-1.34 (m, 4H), 1.25 (s, 1H), 1.18-1.23 (m, 3H), 1.15 (d, J=6.20 Hz, 2H), 0.96 (d, J=6.20 Hz, 1H), 0.68 (dd, J=9.54, 5.72 Hz.

The following examples in Table ZZ were prepared according to scheme VB—S using the procedures outlined in the synthesis of Example 327.

TABLE ZZ

| | | | MS |
|---|---|---|---|
| Ex | Structure | Name | (M+1) |
| 407 | 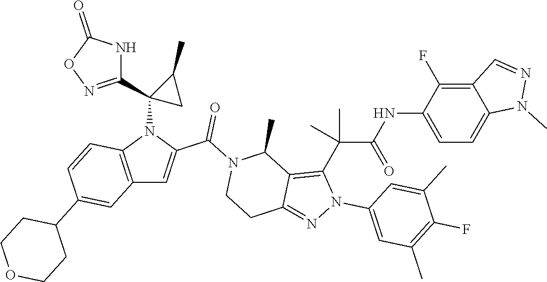 [SMILES: Cc1cc(-n2nc3c(c2C(C)(C)C(=O)Nc2ccc4c(cnn4C)c2F)[C@H](C)N(C(=O)c2cc4cc(C5CCOCC5)ccc4n2[C@@]2(c4noc(=O)[nH]4)C[C@@H]2C)CC3)cc(C)c1F] | N-(4-fluoro-1-methyl-1H-indazol-5- yl)-2-((S)-2-(4-fluoro-3,5- dimethylphenyl)-4-methyl-5-(1- ((1S,2S)-2-methyl-1-(5-oxo-4,5- dihydro-1,2,4-oxadiazol-3- yl)cyclopropyl)-5-(tetrahydro-2H- pyran-4-yl)-1H-indole-2-carbonyl)- 4,5,6,7-tetrahydro-2H-pyrazolo[4,3- c]pyridin-3-yl)-2- methylpropanamide | 858.4 |
| 411 | 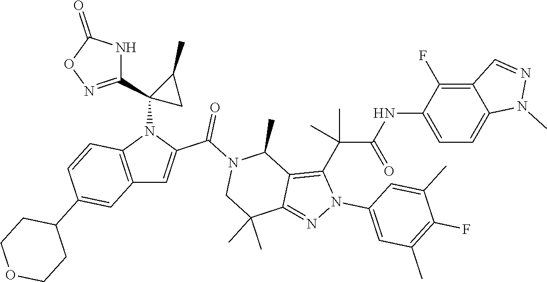 [SMILES: Cc1cc(-n2nc3c(c2C(C)(C)C(=O)Nc2ccc4c(cnn4C)c2F)[C@H](C)N(C(=O)c2cc4cc(C5CCOCC5)ccc4n2[C@@]2(c4noc(=O)[nH]4)C[C@@H]2C)CC3(C)C)cc(C)c1F] | N-(4-fluoro-1-methyl-1H-indazol-5- yl)-2-((S)-2-(4-fluoro-3,5- dimethylphenyl)-4,7,7-trimethyl-5-(1- ((1S,2S)-2-methyl-1-(5-oxo-4,5- dihydro-1,2,4-oxadiazol-3- yl)cyclopropyl)-5-(tetrahydro-2H- pyran-4-yl)-1H-indole-2-carbonyl)- 4,5,6,7-tetrahydro-2H-pyrazolo[4,3- c]pyridin-3-yl)-2- methylpropanamide | 885.2 |
| 412 | 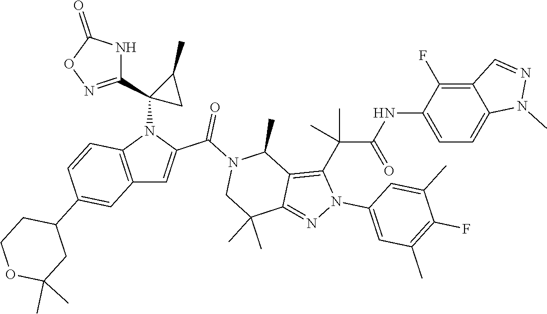 [SMILES: Cc1cc(-n2nc3c(c2C(C)(C)C(=O)Nc2ccc4c(cnn4C)c2F)[C@H](C)N(C(=O)c2cc4cc(C5CCOC(C)(C)C5)ccc4n2[C@@]2(c4noc(=O)[nH]4)C[C@@H]2C)CC3(C)C)cc(C)c1F] | 2-((S)-5-(5-((S)-2,2- dimethyltetrahydro-2H-pyran- 4-yl)-1-((1S,2S)-2-methyl-1- (5-oxo-4,5-dihydro-1,2,4- oxadiazol-3-yl)cyclopropyl)-1H- indole-2-carbonyl)-2-(4-fluoro-3,5- dimethylphenyl)-4,7,7-trimethyl- 4,5,6,7-tetrahydro-2H-pyrazolo[4,3- c]pyridin-3-yl)-N-(4-fluoro-1- methyl-1H-indazol-5-yl)-2- methylpropanamide | 914.4 |

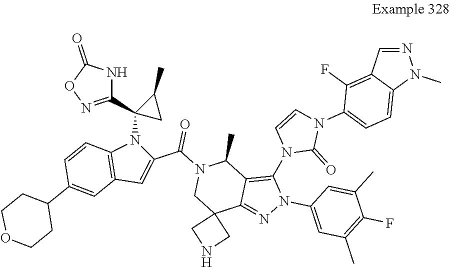 [SMILES: Cc1cc(-n2nc3c(c2-n2ccn(-c4ccc5c(cnn5C)c4F)c2=O)[C@H](C)N(C(=O)c2cc4cc(C5CCOCC5)ccc4n2[C@@]2(c4noc(=O)[nH]4)C[C@@H]2C)CC32CNC2)cc(C)c1F]

### 3-((1S,2S)-1-(2-((4′S)-3′-(3-(4-fluoro-1-methyl-1H-indazol-5-yl)-2-oxo-2,3-dihydro-1H-imidazol-1-yl)-2′-(4-fluoro-3,5-dimethylphenyl)-4′-methyl-2′,4′,5′,6′-tetrahydrospiro[azetidine-3,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one

### Step 1: 5′-benzyl 1-(tert-butyl) (S)-3′-(3-(2,2-dimethoxyethyl)-3-(4-fluoro-1-methyl-1H-indazol-5-yl)ureido)-2′-(4-fluoro-3,5-dimethylphenyl)-4′-methyl-2′,4′-dihydrospiro[azetidine-3,7′-pyrazolo[4,3-c]pyridine]-1,5′(6′H)-dicarboxylate

A solution of 5′-benzyl 1-(tert-butyl) (S)-3′-amino-2′-(4-fluoro-3,5-dimethylphenyl)-4′-methyl-2′,4′-dihydrospiro[azetidine-3,7′-pyrazolo[4,3-c]pyridine]-1,5′(6′H)-dicarboxylate (630 mg, 1.15 mmol) in THF (3.8 mL) was added dropwise to a solution of bis(trichloromethyl) carbonate (323 mg, 1.09 mmol) and DIPEA (1.04 g, 1.40 mL, 8.02 mmol) in THF (3.8 mL) kept at 0° C. in a flame-dried round-bottom flask. The reaction mixture was stirred at 0° C. for 2 h. Afterwards, a solution of N-(2,2-dimethoxyethyl)-4-fluoro-1-methyl-1H-indazol-5-amine (408 mg, 1.61 mmol) in THF (4.0 mL) was added dropwise at 0° C. The reaction mixture was slowly warmed to rt and stirred for 1 h. The mixture was then treated with 1M aqueous K₃PO₄ (20 mL) and EtOAc (20 mL). After mixing, the aqueous phase was extracted with EtOAc (2×20 mL). The combined organic layers were dried over MgSO₄, filtered, and concentrated under reduced pressure. The residue was purified via silica gel column chromatography (25-70% EtOAc/hexanes) to provide the title compound. MS (ESI) m/z: 829.7 [M+H]⁺.

### Step 2: 5′-benzyl 1-(tert-butyl) (4′S)-3′-(3-(4-fluoro-1-methyl-1H-indazol-5-yl)-2-oxo-2,3-dihydro-1H-imidazol-1-yl)-2′-(4-fluoro-3,5-dimethylphenyl)-4′-methyl-2′,4′-dihydrospiro[azetidine-3,7′-pyrazolo[4,3-c]pyridine]-1,5′(6′H)-dicarboxylate

A solution of 5′-benzyl 1-(tert-butyl) (S)-3′-(3-(2,2-dimethoxyethyl)-3-(4-fluoro-1-methyl-1H-indazol-5-yl)ureido)-2′-(4-fluoro-3,5-dimethylphenyl)-4′-methyl-2′,4′-dihydrospiro[azetidine-3,7′-pyrazolo[4,3-c]pyridine]-1,5′(6′H)-dicarboxylate (590 mg, 0.71 mmol) in dioxane (10 mL) was added to a solution of 4-methylbenzenesulfonic acid hydrate (149 mg, 0.78 mmol) in dioxane (25 mL) kept at 40° C. Mixture was stirred 40° C. for 18 h. Afterwards, 1M aqueous K₂CO₃ (30 mL), brine (30 mL) and EtOAc (30 mL) were added. After mixing, the aqueous phase was extracted with EtOAc (4×25 mL). The combined organic layers were washed with brine (25 mL), dried over MgSO₄, filtered, and concentrated under reduced pressure. The residue was purified via silica gel column chromatography (30-60% EtOAc/hexanes) to provide the title compound. MS (ESI) m/z: 665.6 [M−Boc+H]⁺.

### Step 3: tert-butyl (4′S)-3′-(3-(4-fluoro-1-methyl-1H-indazol-5-yl)-2-oxo-2,3-dihydro-1H-imidazol-1-yl)-2′-(4-fluoro-3,5-dimethylphenyl)-4′-methyl-2′,4′,5′,6′-tetrahydrospiro[azetidine-3,7′-pyrazolo]4,3-c]pyridine]-1-carboxylate

Triethylsilane (160 mg, 0.23 mL, 1.4 mmol) was added to a solution of 5′-benzyl 1-(tert-butyl) (4′S)-3′-(3-(4-fluoro-1-methyl-1H-indazol-5-yl)-2-oxo-2,3-dihydro-1H-imidazol-1-yl)-2′-(4-fluoro-3,5-dimethylphenyl)-4′-methyl-2′,4′-dihydrospiro[azetidine-3,7′-pyrazolo[4,3-c]pyridine]-1,5′(6′H)-dicarboxylate (270 mg, 0.35 mmol), triethylamine (39 mg, 54 μL, 0.39 mmol) and palladium(II) chloride (3.1 mg, 0.018 mmol) in THF (5.0 mL) under N₂. The mixture was stirred at rt for 1 h. The mixture was then diluted with MeCN (10 mL) and sequentially filtered through Celite® and a 45 μm syringe filter. The filtrate was diluted with toluene (40 mL) and concentrated under reduced pressure to yield the title compound, which was used in the next step without purification. MS (ESI) m/z: 631.6 [M+H]⁺.

### Step 4: tert-butyl (4′S)-3′-(3-(4-fluoro-1-methyl-1H-indazol-5-yl)-2-oxo-2,3-dihydro-1H-imidazol-1-yl)-2′-(4-fluoro-3,5-dimethylphenyl)-4′-methyl-5′-(1-((2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-2′,4′,5′,6′-tetrahydrospiro[azetidine-3,7′-pyrazolo[4,3-c]pyridine]-1-carboxylate

DIPEA (110 mg, 0.15 mL, 0.88 mmol) was added to a mixture of 1-((2S)-2-methyl-1-(5-oxo-4,5-dihydro-1,2,4-oxadiazol-3-yl)cyclopropyl)-5-(tetrahydro-2H-pyran-4-yl)-1H-indole-2-carboxylic acid (160 mg, 0.41 mmol), tert-butyl (4′S)-3′-(3-(4-fluoro-1-methyl-1H-indazol-5-yl)-2-oxo-2,3-dihydro-1H-imidazol-1-yl)-2′-(4-fluoro-3,5-dimethylphenyl)-4′-methyl-2′,4′,5′,6′-tetrahydrospiro[azetidine-3,7′-pyrazolo[4,3-c]pyridine]-1-carboxylate (370 mg, 50% wt, 0.29 mmol) and HATU (160 mg, 0.41 mmol) in DMA (7.4 mL). The reaction mixture was stirred at 60° C. for 1.5 h. The mixture was then diluted with EtOAc (40 mL), washed with brine (4×5 mL), dried over MgSO₄, filtered, and concentrated under reduced pressure. The residue was purified via silica gel column chromatography (50-90% EtOAc/hexanes) followed by purification via C18 reverse phase chromatography (30-95% MeCN/water) to provide the title compound. MS (ESI) m/z: 896.7 [M−Boc+H]⁺.

### Step 5: 3-((2S)-1-(2-((4′S)-3′-(3-(4-fluoro-1-methyl-1H-indazol-5-yl)-2-oxo-2,3-dihydro-1H-imidazol-1-yl)-2′-(4-fluoro-3,5-dimethylphenyl)-4′-methyl-2′,4′,5′,6′-tetrahydrospiro[azetidine-3,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one

Trifluoroacetic acid (2.2 g, 1.5 mL, 19 mmol) was added dropwise to a solution of tert-butyl (4′S)-3′-(3-(4-fluoro-1-methyl-1H-indazol-5-yl)-2-oxo-2,3-dihydro-1H-imidazol-1-yl)-2′-(4-fluoro-3,5-dimethylphenyl)-4′-methyl-5′-(1-((2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-2′,4′,5′,6′-tetrahydrospiro[azetidine-3,7′-pyrazolo[4,3-c]pyridine]-1-carboxylate (150 mg, 0.15 mmol) in DCM (1.5 mL) kept at 0° C. The mixture was stirred at 0° C. for 30 min. Afterwards, 1M aqueous K₃PO₄ (5 mL) was added, followed by saturated aqueous K₂CO₃ (until pH is over 9, ˜6 mL). The aqueous phase was filtered and extracted with EtOAc (4×10 mL). The combined organic layers were washed with 1M aqueous K₃PO₄ (5 mL) and brine (3×5 mL), combined with the solid residue from the previous filtration (redissolved with 2 mL MeCN), dried over MgSO₄, filtered, and concentrated under reduced pressure. The residue was purified via C18 reverse phase chromatography (30-95% MeCN/water with 10 mM ammonium bicarbonate modifier) to provide the title compound. ¹H NMR (400 MHz, CH₃CN-d₃, rotamers): δ_{H} 8.15 (0.5H, s), 8.05-8.07 (0.5H, m), 7.72 (0.5H, s), 7.59 (0.5H, d, J=8.6 Hz), 7.45-7.50 (2H, m), 7.15-7.36 (4H, m), 6.98-7.03 (1H, m), 6.84 (0.5H, br s), 6.72 (0.5H, s), 6.54-6.60 (1H, m), 6.33-6.47 (1H, m), 5.74 (0.5H, q, J=6.8 Hz), 5.29-5.54 (0.5H, m), 4.63-4.4.72 (1H, m), 4.20-4.36 (1H, m), 4.10 (2H, s), 4.04 (1H, s), 3.90-4.02 (3H, m), 3.29-3.61 (3H, m), 2.81-3.07 (2H, m), 2.53 (1H, s), 2.26-2.29 (5H, m), 2.19 (2H, s), 1.68-1.77 (4H, m), 1.47-1.60 (3H, m), 1.38 (3H, dd, J=6.5, 3.9 Hz), 1.30 (2H, s), 1.02 (1H, d, J=73.6 Hz). ¹⁹F NMR (376 MHz, CH₃CN-d₃, rotamers): δ_{F} −122.5-−122.9 (m; 1 F); −127.8-−127.9 (m, 1F). MS (ESI) m/z: 896.4 [M+H]⁺.

The following intermediates in Table 30 were prepared using the procedures outlined in the synthesis of Example 328.

TABLE 30

| | | | MS |
|---|---|---|---|
| Intermediate | Structure | Name | (M+H) |
| 329 | 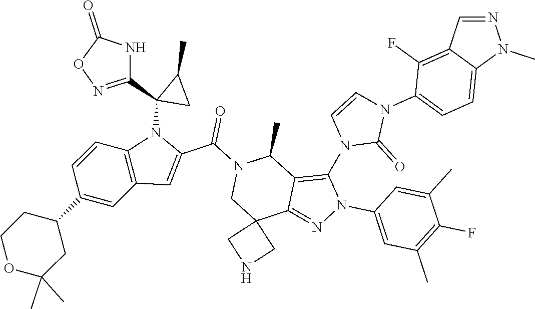 [SMILES: Cc1cc(-n2nc3c(c2-n2ccn(-c4ccc5c(cnn5C)c4F)c2=O)[C@H](C)N(C(=O)c2cc4cc([C@H]5CCOC(C)(C)C5)ccc4n2[C@@]2(c4noc(=O)[nH]4)C[C@@H]2C)CC32CNC2)cc(C)c1F] | 3-((1S,2S)-1-(5-((S)-2,2- dimethyltetrahydro-2H- pyran-4-yl)-2-((4′S)-3′-(3- (4-fluoro-1-methyl-1H- indazol-5-yl)-2-oxo-2,3- dihydro-1H-dimethylphenyl)- 4′-methyl-2′,4′,5′,6′- imidazol-1-yl)-2′-(4-fluoro-3,5- tetrahydrospiro[azetidine-3,7′- pyrazolo[4,3-c]pyridine]-5′- carbonyl)-1H-indol-1-yl)-2- methylcyclopropyl)- 1,2,4-oxadiazol-5(4H)-one | 924.6 |

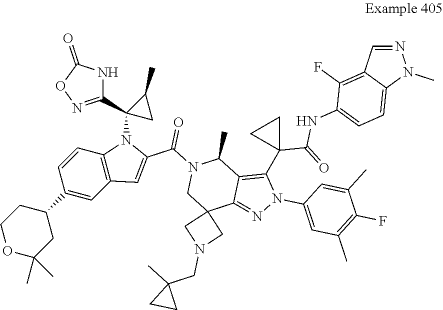 [SMILES: Cc1cc(-n2nc3c(c2C2(C(=O)Nc4ccc5c(cnn5C)c4F)CC2)[C@H](C)N(C(=O)c2cc4cc([C@H]5CCOC(C)(C)C5)ccc4n2[C@@]2(c4noc(=O)[nH]4)C[C@@H]2C)CC32CN(CC3(C)CC3)C2)cc(C)c1F]

### 1-((S)-5′-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2′-(4-fluoro-3,5-dimethylphenyl)-4′-methyl-1-((1-methylcyclopropyl)methyl)-2′,4′,5′,6′-tetrahydrospiro[azetidine-3,7′-pyrazolo[4,3-c]pyridin]-3′-yl)-N-(4-fluoro-1-methyl-1H-indazol-5-yl)cyclopropane-1-carboxamide

### Step 1: 5′-benzyl 1-(tert-butyl) (S)-2′-(4-fluoro-3,5-dimethylphenyl)-3′-(1-(methoxycarbonyl)cyclopropyl)-4′-methyl-2′,4′-dihydrospiro[azetidine-3,7′-pyrazolo[4,3-c]pyridine]-1,5′(6′H)-dicarboxylate

To a solution of 5′-benzyl 1-(tert-butyl) (S)-3′-bromo-2′-(4-fluoro-3,5-dimethylphenyl)-4′-methyl-2′,4′-dihydrospiro[azetidine-3,7′-pyrazolo[4,3-c]pyridine]-1,5′(6′H)-dicarboxylate (1200 mg, 1.96 mmol), bis[tris(tert-butyl)phosphine]palladium(0) (100 mg, 196 mol) in THF (12 mL) was added (1-(methoxycarbonyl)cyclopropyl)zinc(II) bromide (2.39 g, 37.6 mL, 0.26 molar, 9.8 mmol) under an atmosphere of nitrogen. The reaction mixture was warmed to 60° C. and stirred at 60° C. for 16 hours under nitrogen. The reaction mixture was quenched with sat. aq. ammonium chloride (20 mL), and extracted with EtOAc (3×20 mL). The organic layers were combined, dried over Na₂SO₄, filtered, and concentrated under reduced pressure to provide the crude product which was purified by silica gel chromatography (0-35% EtOAc/pet. ether) to provide the title compound. MS (ESI) m/z: 633.3 [M+H⁺].

### Step 2: benzyl (S)-2′-(4-fluoro-3,5-dimethylphenyl)-3′-(1-(methoxycarbonyl) cyclopropyl)-4′-methyl-2′,4′-dihydrospiro[azetidine-3,7′-pyrazolo[4,3-c]pyridine]-5′(6′H)-carboxylate

A solution of 5′-benzyl 1-(tert-butyl) (S)-2′-(4-fluoro-3,5-dimethylphenyl)-3′-(1-(methoxycarbonyl)cyclopropyl)-4′-methyl-2′,4′-dihydrospiro[azetidine-3,7′-pyrazolo[4,3-c]pyridine]-1,5′(6′H)-dicarboxylate (1.13 g, 1.79 mmol) in DCM (10 mL) and TFA (3 mL) was stirred at 20° C. for 16 hours. The reaction mixture was quenched with sat. aq. NaHCO₃ (20 mL), and extracted with DCM (3×15 mL). The organic layers were combined, dried over Na₂SO₄, filtered, and concentrated under reduced pressure to provide the title compound which was used directly in the next step without purification. MS (ESI) m/z: 533.4 [M+H⁺].

### Step 3: benzyl (S)-2′-(4-fluoro-3,5-dimethylphenyl)-3′-(1-(methoxycarbonyl) cyclopropyl)-4′-methyl-1-((1-methylcyclopropyl)methyl)-2′,4′-dihydrospiro[azetidine-3,7′-pyrazolo[4,3-c]pyridine]-5′(6′H)-carboxylate

To a mixture of benzyl (S)-2′-(4-fluoro-3,5-dimethylphenyl)-3′-(1-(methoxycarbonyl) cyclopropyl)-4′-methyl-2′,4′-dihydrospiro[azetidine-3,7′-pyrazolo[4,3-c]pyridine]-5′(6′H)-carboxylate (260 mg, 488 mol) and 1-methylcyclopropane-1-carbaldehyde (246 mg, 50% wt, 1.46 mmol) in DCE (6 mL) was added AcOH (58.6 mg, 55.9 μL, 976 mol). After stirring for 5 minutes at 20° C., sodium triacetoxyborohydride (310 mg, 217 μL, 1.46 mmol) was added. The resultant mixture was stirred at 20° C. for 16 hours. The mixture was filtered and concentrated to afford the crude residue which was purified by reverse phase HPLC (YMC-Actus Triart C18 H₂O(0.1% TFA)-ACN, 40-100%) to provide the title compound. MS (ESI) m/z: 601.7 [M+H⁺].

### Step 4: (S)-1-(5′-((benzyloxy)carbonyl)-2′-(4-fluoro-3,5-dimethylphenyl)-4′-methyl-1-((1-methylcyclopropyl)methyl)-2′,4′,5′,6′-tetrahydrospiro[azetidine-3,7′-pyrazolo[4,3-c]pyridin]-3′-yl)cyclopropane-1-carboxylic acid

This step was performed in a similar fashion to that of Example 281, step 3 to provide the title compound. MS (ESI) m/z: 587.7 [M+H⁺].

### Step 5: benzyl (S)-3′-(1-((4-fluoro-1-methyl-1H-indazol-5-yl)carbamoyl)cyclopropyl)-2′-(4-fluoro-3,5-dimethylphenyl)-4′-methyl-1-((1-methylcyclopropyl)methyl)-2′,4′-dihydrospiro[azetidine-3,7′-pyrazolo[4,3-c]pyridine]-5′(6′H)-carboxylate

This step was performed in a similar fashion to that of Example 281, step 4 to provide the title compound. MS (ESI) m/z: 734.4 [M+H⁺].

### Step 6: (S)—N-(4-fluoro-1-methyl-1H-indazol-5-yl)-1-(2′-(4-fluoro-3,5-dimethylphenyl)-4′-methyl-1-((1-methylcyclopropyl)methyl)-2′,4′,5′,6′-tetrahydrospiro[azetidine-3,7′-pyrazolo[4,3-c]pyridin]-3′-yl)cyclopropane-1-carboxamide

To a mixture of (S)—N-(4-fluoro-1-methyl-1H-indazol-5-yl)-1-(2′-(4-fluoro-3,5-dimethylphenyl)-4′-methyl-1-((1-methylcyclopropyl)methyl)-2′,4′,5′,6′-tetrahydrospiro[azetidine-3,7′-pyrazolo[4,3-c]pyridin]-3′-yl)cyclopropane-1-carboxamide (45.0 mg, 75.0 mol) and PdCl₂ (1.33 mg, 391 nL, 7.50 mol) in DCM (6 mL) was added TEA (22.8 mg, 31.4 μL, 225 mol) and triethylsilane (105 mg, 900 mol). After stirring for 16 hours at 20° C., the mixture was filtered and concentrated under reduced pressure to afford the crude residue, which was purified by reverse phase HPLC (YMC-Actus Triart C18 H₂O (0.1% TFA)-ACN 15-100%) to provide the title compound. MS (ESI) m/z: 600.4 [M+H⁺].

### Step 7: 1-((S)-5′-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2′-(4-fluoro-3,5-dimethylphenyl)-4′-methyl-1-((1-methylcyclopropyl)methyl)-2′,4′,5′,6′-tetrahydrospiro[azetidine-3,7′-pyrazolo[4,3-c]pyridin]-3′-yl)-N-(4-fluoro-1-methyl-1H-indazol-5-yl)cyclopropane-1-carboxamide

This reaction was performed in a similar fashion as to Example 1, step 5 to provide the title compound. MS (ESI) m/z: 993.4 [M+H⁺]. ¹H NMR (400 MHz, ACN-d₃) δ ppm 10.81-11.32 (m, 2H), 7.87 (br s, 2H), 7.68 (s, 1H), 7.47-7.57 (m, 1H), 7.25-7.45 (m, 4H), 7.03-7.13 (m, 1H), 5.53-5.77 (m, 1H), 5.13-5.49 (m, 1H), 4.86-5.08 (m, 1H), 4.62-4.75 (m, 1 H), 4.01-4.06 (m, 1H), 3.98 (s, 3H), 3.71-3.86 (m, 3H), 3.60-3.69 (m, 1H), 3.29 (br s, 1H), 3.08-3.18 (m, 1H), 2.33 (s, 6H), 1.90 (br d, J=6.56 Hz, 3H), 1.73-1.79 (m, 3H), 1.68-1.72 (m, 1H), 1.57-1.64 (m, 2H), 1.31 (s, 5H), 1.20 (s, 4H), 1.16 (br s, 3H), 0.97 (br d, J=4.17 Hz, 3H), 0.80-0.88 (m, 1H), 0.70-0.76 (m, 1H), 0.56-0.67 (m, 2H), 0.46-0.51 (m, 2H).

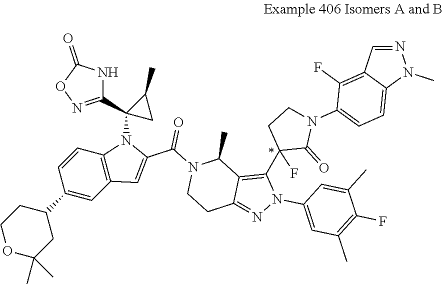

### 3-((1 S,2S)-1-(5-((S)-2,2-dimethyltetrahydro-2H-pyran-4-yl)-2-((S)-3-((R)-3-fluoro-1-(4-fluoro-1-methyl-1H-indazol-5-yl)-2-oxopyrrolidin-3-yl)-2-(4-fluoro-3,5-dimethylphenyl)-4-methyl-4,5,6,7-tetrahydro-2H-pyrazolo[4,3-c]pyridine-5-carbonyl)-1H-indol-1l-yl)-2-methylcyclopropyl)-1,2,4-oxadiazol-5(4H)-one AND 3-((1S,2S)-1-(5-((S)-2,2-dimethyltetrahydro-2H-pyran-4-yl)-2-((S)-3-((S)-3-fluoro-1-(4-fluoro-1-methyl-1H-indazol-5-yl)-2-oxopyrrolidin-3-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one

### Step 1: tert-butyl (4S)-3-(4-(((benzyloxy)carbonyl)amino)-1-methoxy-1-oxobutan-2-yl)-2-(4-fluoro-3,5-dimethylphenyl)-4-methyl-2,4,6,7-tetrahydro-5H-pyrazolo[4,3-c]pyridine-5-carboxylate

A solution of tert-butyl (S)-2-(4-fluoro-3,5-dimethylphenyl)-3-(3-methoxy-3-oxoprop-1-en-2-yl)-4-methyl-2,4,6,7-tetrahydro-5H-pyrazolo[4,3-c]pyridine-5-carboxylate (398 mg, 897 mol), K₂HPO₄ (188 mg, 1.08 mmol), (4,4′-Di-t-butyl-2,2′-bipyridine)bis[3,5-difluoro-2-(5-trifluoromethyl-2-pyridinyl-kN)phenyl-kC]iridium(III) hexafluorophosphate (10.1 mg, 8.97 mol) and ((benzyloxy)carbonyl)glycine (225 mg, 1.08 mmol) in DMF (6 mL) was stirred at 25° C. for 16 hours under 450 nm blue LED and nitrogen. The reaction mixture was quenched with NaHCO₃ sat. aq. (10 mL) and extracted with EtOAc (3×15 mL). The combined organics were washed with brine (20 mL), dried over Na₂SO₄, filtered, and concentrated under reduced pressure to provide crude product, which was purified by silica gel chromatography (hexane/EtOAc 75%) to provide the title compound. MS (ESI) m/z: 609.4 [M+H]⁺.

### Step 2: tert-butyl (4S)-2-(4-fluoro-3,5-dimethylphenyl)-4-methyl-3-(2-oxopyrrolidin-3-yl)-2,4,6,7-tetrahydro-5H-pyrazolo[4,3-c]pyridine-5-carboxylate

Tert-butyl (4S)-3-(4-(((benzyloxy)carbonyl)amino)-1-methoxy-1-oxobutan-2-yl)-2-(4-fluoro-3,5-dimethylphenyl)-4-methyl-2,4,6,7-tetrahydro-5H-pyrazolo[4,3-c]pyridine-5-carboxylate (75.00 mg, 1 Eq, 123.2 μmol) in MeOH (5 mL) was passed over Pd/C granular catalyst under 1 MPa of H₂ in a flow reactor (flow rate=30 mL/min) at 50.0° C. The reaction mixture was continuously collected from the reactor outlet, filtered, and concentrated to provide the crude title compound which was used in the next step without further purification. MS (ESI) m/z: 475.2 [M+H]⁺.

### Step 3: tert-butyl (4S)-2-(4-fluoro-3,5-dimethylphenyl)-4-methyl-3-(2-oxopyrrolidin-3-yl)-2,4,6,7-tetrahydro-5H-pyrazolo[4,3-c]pyridine-5-carboxylate

TEA (297 mg, 409 μL, 2.93 mmol) was added to solution of tert-butyl (4S)-3-(4-amino-1-methoxy-1-oxobutan-2-yl)-2-(4-fluoro-3,5-dimethylphenyl)-4-methyl-2,4,6,7-tetrahydro-5H-pyrazolo[4,3-c]pyridine-5-carboxylate (116 mg, 244 mol) in THF (2 mL) at 25° C. The reaction mixture was heated to 100° C. for 2 hours under microwave irradiation. The reaction mixture was filtered and concentrated under reduced pressure to provide crude product, which was purified by reverse phase HPLC (YMC-Actus Triart C18, H₂O(0.1% TFA)-ACN 35-100%) to provide the title compound. MS (ESI) m/z: 443.2 [M+H]⁺.

### Step 4: tert-butyl (4S)-3-(1-(4-fluoro-1-methyl-1H-indazol-5-yl)-2-oxopyrrolidin-3-yl)-2-(4-fluoro-3,5-dimethylphenyl)-4-methyl-2,4,6,7-tetrahydro-5H-pyrazolo[4,3-c]pyridine-5-carboxylate

To a solution of tert-butyl (4S)-2-(4-fluoro-3,5-dimethylphenyl)-4-methyl-3-(2-oxopyrrolidin-3-yl)-2,4,6,7-tetrahydro-5H-pyrazolo[4,3-c]pyridine-5-carboxylate (55.3 mg, 125 mol), 5-bromo-4-fluoro-1-methyl-1H-indazole (57.3 mg, 250 μmol), K₃PO₄ (79.6 mg, 31.1 μL, 375 μmol) and CuI (23.8 mg, 125 mol) in toluene (3 mL) was added N1,N2-dimethylethane-1,2-diamine (22.1 mg, 250 mol) at 25° C. The mixture was stirred at 110° C. for 16 hours. To the reaction mixture was added water and the mixture was extracted with EtOAc (3×10 mL). The combined organic layers were washed with brine (10 mL), dried over Na₂SO₄, filtered, and concentrated under reduced pressure to provide crude product, which was purified by prep-TLC (SiO₂, Hexane:EtOAc=1:1) to provide the title compound. MS (ESI) m/z calc'd: 591.2 [M+H]⁺.

### Step 5: tert-butyl (4S)-3-(1-(4-fluoro-1-methyl-1H-indazol-5-yl)-3-hydroxy-2-oxopyrrolidin-3-yl)-2-(4-fluoro-3,5-dimethylphenyl)-4-methyl-2,4,6,7-tetrahydro-5H-pyrazolo[4,3-c]pyridine-5-carboxylate

To a solution of tert-butyl (4S)-3-(1-(4-fluoro-1-methyl-1H-indazol-5-yl)-2-oxopyrrolidin-3-yl)-2-(4-fluoro-3,5-dimethylphenyl)-4-methyl-2,4,6,7-tetrahydro-5H-pyrazolo[4,3-c]pyridine-5-carboxylate (42.0 mg, 71.1 μmol) in THF (2 mL) was added TBAF (186 mg, 7.11 mL, 0.1 M, 711 mol) at 25° C. under air. The reaction mixture was stirred at 25° C. for 16 hours. The mixture was quenched with sat. aq. NH₄Cl (5 mL), extracted with EtOAc (3×10 mL), washed with brine (5 mL), dried over Na₂SO₄, filtered, and concentrated under reduced pressure to provide the crude title compound, which was used in the next step without further purification. MS(ESI)m/z: 607.2 (M+H)

### Step 6: tert-butyl (S)-3-((S)-3-fluoro-1-(4-fluoro-1-methyl-1H-indazol-5-yl)-2-oxopyrrolidin-3-yl)-2-(4-fluoro-3,5-dimethylphenyl)-4-methyl-2,4,6,7-tetrahydro-5H-pyrazolo[4,3-c]pyridine-5-carboxylate and tert-butyl (S)-3-((S)-3-fluoro-1-(4-fluoro-1-methyl-1H-indazol-5-yl)-2-oxopyrrolidin-3-yl)-2-(4-fluoro-3,5-dimethylphenyl)-4-methyl-2,4,6,7-tetrahydro-5H-pyrazolo[4,3-c]pyridine-5-carboxylate

To a solution of tert-butyl (4S)-3-(1-(4-fluoro-1-methyl-1H-indazol-5-yl)-3-hydroxy-2-oxopyrrolidin-3-yl)-2-(4-fluoro-3,5-dimethylphenyl)-4-methyl-2,4,6,7-tetrahydro-5H-pyrazolo[4,3-c]pyridine-5-carboxylate (21.4 mg, 35.3 μmol) in DCM (2 mL) was added N,N-diethyl-1,1,1-trifluoro-14-sulfanamine (11.4 mg, 70.5 μmol) at 0° C. The resultant mixture was stirred at 20° C. for 1 hour under nitrogen. The mixture was quenched by sat. aq. NaHCO₃ (8 mL) and extracted with DCM (3×10 mL). The combined organic layer was washed with brine, dried with Na₂SO₄, filtered, and concentrated under reduced pressure. The crude residue was purified by reverse phase HPLC (YMC-Actus Triart C18, H₂O (0.1% TFA)-ACN 55-100%) to provide the two isomers of the title compound. Isomer A: MS(ESI)m/z: 609.7 (M+H). Isomer B: MS(ESI)m/z: 609.7 (M+H).

### Step 7: 3-fluoro-1-(4-fluoro-1-methyl-1H-indazol-5-yl)-3-((S)-2-(4-fluoro-3,5-dimethylphenyl)-4-methyl-4,5,6,7-tetrahydro-2H-pyrazolo[4,3-c]pyridin-3-yl)pyrrolidin-2-one

This step was performed in a similar fashion as to that in Example 1, step 4 to provide the title compound. MS (ESI) m/z [M+H]⁺: 509.1

### Step 8: 3-((1S,2S)-1-(5-((S)-2,2-dimethyltetrahydro-2H-pyran-4-yl)-2-((4S)-3-(3-fluoro-1-(4-fluoro-1-methyl-1H-indazol-5-yl)-2-oxopyrrolidin-3-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one

This step was performed in a similar fashion as to that in Example 1, step 5 to provide the title compound. Isomer A (Step 6) MS (ESI) m/z calc'd: 902.3 [M+H]⁺. ¹H NMR (400 MHz, ACN-d₃) δ11.65-11.43 (m, 1H), 8.09-7.96 (m, 1H), 7.55-7.44 (m, 2H), 7.42-7.34 (m, 1H), 7.28-7.21 (m, 1H), 7.12-6.93 (m, 3H), 6.85-6.78 (m, 1H), 5.97 (q, J=6.2 Hz, 1H), 5.55-5.42 (m, 1H), 4.74 (br dd, J=6.1, 13.4 Hz, 1H), 4.36 (br dd, J=5.7, 14.1 Hz, 1H), 4.06-3.99 (m, 3H), 3.91-3.80 (m, 1H), 3.78-3.67 (m, 3H), 3.53-3.43 (m, 1H), 3.12-2.99 (m, 3H), 2.96-2.84 (m, 2H), 2.80 (br t, J=6.6 Hz, 1H), 2.72 (br t, J=6.6 Hz, 1H), 2.27-2.20 (m, 6H), 1.82-1.74 (m, 2H), 1.69 (br s, 1H), 1.63 (br s, 3H), 1.59-1.51 (m, 2H), 1.29 (s, 3H), 1.20-1.14 (m, 5H), 0.92 (d, J=6.0 Hz, 1H). Isomer B (Step 6) MS (ESI) m/z calc'd: 902.2 [M+H]⁺. ¹H NMR (400 MHz, ACN-d₃) δ 11.60-11.45 (m, 1H), 8.13-8.08 (m, 1H), 7.55-7.46 (m, 2H), 7.42 (dd, J=4.1, 8.8 Hz, 1H), 7.34-7.25 (m, 1H), 7.24-7.16 (m, 1H), 7.09-7.02 (m, 2H), 6.82 (s, 1H), 6.02 (q, J=6.5 Hz, 1H), 5.55 (q, J=6.4 Hz, 1H), 4.83-4.67 (m, 1H), 4.39-4.29 (m, 1H), 4.05 (d, J=10.8 Hz, 3H), 3.92-3.83 (m, 1H), 3.81-3.69 (m, 3H), 3.69-3.52 (m, 1H), 3.50-3.36 (m, 1H), 3.15-2.86 (m, 3H), 2.69-2.56 (m, 1H), 2.56-2.47 (m, 1H), 2.45-2.40 (m, 1H), 2.11 (td, J=2.4, 5.0 Hz, 1H), 1.84-1.78 (m, 1H), 1.77-1.75 (m, 1H), 1.74-1.68 (m, 3H), 1.67-1.62 (m, 3H), 1.60-1.53 (m, 2H), 1.47 (s, 1H), 1.44-1.34 (m, 1H), 1.31-1.26 (m, 4H), 1.24-1.03 (m, 6H), 0.99 (d, J=6.1 Hz, 1H)

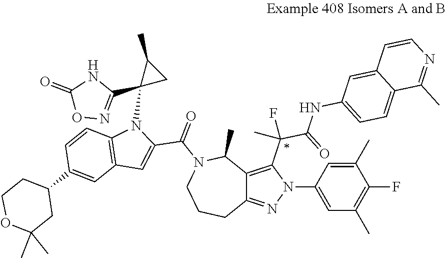 [SMILES: Cc1cc(-n2nc3c(c2C(C)(F)C(=O)Nc2ccc4c(C)nccc4c2)[C@H](C)N(C(=O)c2cc4cc([C@H]5CCOC(C)(C)C5)ccc4n2[C@@]2(c4noc(=O)[nH]4)C[C@@H]2C)CCC3)cc(C)c1F]

### (S)-2-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-2,4,5,6,7,8-hexahydropyrazolo[4,3-c]azepin-3-yl)-2-fluoro-N-(1-methylisoquinolin-6-yl)propenamide AND (R)-2-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-2,4,5,6,7,8-hexahydropyrazolo [4,3-c]azepin-3-yl)-2-fluoro-N-(1-methylisoquinolin-6-yl)propanamide

### Step 1: tert-butyl (S)-3-((R)-1-ethoxy-2-fluoro-1-oxopropan-2-yl)-2-(4-fluoro-3,5-dimethylphenyl)-4-methyl-2,6,7,8-tetrahydropyrazolo[4,3-c]azepine-5(4H)-carboxylate and tert-butyl (S)-3-((S)-1-ethoxy-2-fluoro-1-oxopropan-2-yl)-2-(4-fluoro-3,5-dimethylphenyl)-4-methyl-2,6,7,8-tetrahydropyrazolo[4,3-c]azepine-5(4H)-carboxylate

At −78° C. under a nitrogen atmosphere, a 2.0 M lithium diisopropylamide solution in tetrahydrofuran/heptane/ethylbenzene (181 mg, 845 μL, 2.0 molar, 1.7 mmol) was added to a mixture of tert-butyl (4S)-3-(1-ethoxy-1-oxopropan-2-yl)-2-(4-fluoro-3,5-dimethylphenyl)-4-methyl-2,6,7,8-tetrahydropyrazolo[4,3-c]azepine-5(4H)-carboxylate (400 mg, 845 mol) in anhydrous tetrahydrofuran (2.0 mL). The reaction mixture was stirred at −78° C. for 10 minutes and was then allowed to warm up to 0° C. over the course of 30 minutes. The reaction mixture was again cooled to −78° C. and a solution of N-fluorobis(phenylsulfonyl)amine (399 mg, 1.27 mmol) in anhydrous tetrahydrofuran (2.0 mL) was added. The reaction mixture was allowed to warm to room temperature and stirred for 1 hour. The reaction mixture was diluted with saturated aqueous NH₄Cl and extracted twice with ethyl acetate. The combined organic layers were washed with saturated aqueous NH₄Cl, dried over Na₂SO₄ and concentrated under reduced pressure. The residue obtained was purified by silica gel column chromatography (0-40% EtOAc:heptane) to provide the title compound. LCMS: MS (ESI) m/z: 492.3 [M+H]⁺.

### Step 2: (R)-2-((S)-5-(tert-butoxycarbonyl)-2-(4-fluoro-3,5-dimethylphenyl)-4-methyl-2,4,5,6,7,8-hexahydropyrazolo[4,3-c]azepin-3-yl)-2-fluoropropanoic acid AND (S)-2-((S)-5-(tert-butoxycarbonyl)-2-(4-fluoro-3,5-dimethylphenyl)-4-methyl-2,4,5,6,7,8-hexahydropyrazolo[4,3-c]azepin-3-yl)-2-fluoropropanoic acid

This step was performed in a similar fashion as to that in Example 78, step 4 to provide the title compound. LCMS: MS (ESI) m/z: 464.1 [M+H]⁺.

### Step 3: tert-butyl (S)-3-((R)-2-fluoro-1-((1-methylisoquinolin-6-yl)amino)-1-oxopropan-2-yl)-2-(4-fluoro-3,5-dimethylphenyl)-4-methyl-2,6,7,8-tetrahydropyrazolo[4,3-c]azepine-5(4H)-carboxylate and tert-butyl (S)-3-((S)-2-fluoro-1-((1-methylisoquinolin-6-yl)amino)-1-oxopropan-2-yl)-2-(4-fluoro-3,5-dimethylphenyl)-4-methyl-2,6,7,8-tetrahydropyrazolo[4,3-c]azepine-5(4H)-carboxylate

At room temperature under a nitrogen atmosphere, 2-((S)-5-(tert-butoxycarbonyl)-2-(4-fluoro-3,5-dimethylphenyl)-4-methyl-2,4,5,6,7,8-hexahydropyrazolo[4,3-c]azepin-3-yl)-2-fluoropropanoic acid (287 mg, 619 mol), 1-methylisoquinolin-6-amine (118 mg, 743 mol) and N,N,N′,N′-tetramethylchloroformamidinium-hexafluorophosphate (521 mg, 1.86 mmol) were mixed in anhydrous acetonitrile (5.0 mL). N-methylimidazole (508 mg, 491 μL, 6.19 mmol) was added and the reaction mixture was stirred at room temperature for 3 hours. The reaction mixture was diluted with saturated aqueous NH₄Cl and extracted twice with ethyl acetate. The combined organic layers were dried over Na₂SO₄ and concentrated under reduced pressure. The residue obtained was purified by preparative HPLC (Reprosil-pur Basic C18-HD, 40-80% acetonitrile in water (10 mM NH₄HCO₃ pH 9.5) to provide the title compounds. Isomer A (first eluting): LCMS: MS (ESI) m/z: 604.3 [M+H]⁺. Isomer B (second eluting): LCMS: MS (ESI) m/z: 604.3 [M+H]⁺.

### Step 4: (S)-2-fluoro-2-((S)-2-(4-fluoro-3,5-dimethylphenyl)-4-methyl-2,4,5,6,7,8-hexahydropyrazolo[4,3-c]azepin-3-yl)-N-(1-methylisoquinolin-6-yl)propenamide OR (R)-2-fluoro-2-((S)-2-(4-fluoro-3,5-dimethylphenyl)-4-methyl-2,4,5,6,7,8-hexahydropyrazolo[4,3-c]azepin-3-yl)-N-(1-methylisoquinolin-6-yl)propanamide

This step was performed in a similar fashion as to Example 78, step 2 to provide the title compound. LCMS: MS (ESI) m/z: 504.2 [M+H]⁺.

### Step 5: (S)-2-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-2,4,5,6,7,8-hexahydropyrazolo[4,3-c]azepin-3-yl)-2-fluoro-N-(1-methylisoquinolin-6-yl)propenamide and (R)-2-((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-2,4,5,6,7,8-hexahydropyrazolo[4,3-c]azepin-3-yl)-2-fluoro-N-(1-methylisoquinolin-6-yl)propanamide

This step was performed in a similar fashion as to Example 1, step 5 to provide the title compounds. Isomer A (first eluting, step 3) LCMS: MS (ESI) m/z: 897.7 [M+H]⁺. ¹H NMR (400 MHz, CDCl₃, rotamers) δ 11.09 (bs, 0.3H), 8.74 (d, J=8.4 Hz, 0.5H), 8.50 (d, J=2.2 Hz, 0.5H), 8.43 (d, J=5.8 Hz, 0.4H), 8.35 (d, J=5.8 Hz, 0.6H), 8.10 (dd, J=12.0, 9.0 Hz, 1.0H), 7.93 (d, J=2.1 Hz, 0.4H), 7.66-7.58 (m, 1.0H), 7.51-7.37 (m, 2.7H), 7.33-7.27 (m, 0.8H), 7.23-7.14 (m, 2.2H), 6.97 (d, J=6.2 Hz, 1.0H), 6.56 (s, 0.6H), 6.50 (s, 0.4H), 6.38 (q, J=7.1 Hz, 0.6H), 6.20 (q, J=6.9 Hz, 0.4H), 4.72-4.67 (m, 0.4H), 4.17-4.10 (m, 0.6H), 3.93-3.77 (m, 2.2H), 3.70-3.60 (m, 0.6H), 3.57-3.47 (m, 0.5H), 3.25-3.15 (m, 1.1H), 3.09-2.84 (m, 5.6H), 2.25 (d, J=2.0 Hz, 3.5H), 2.13 (d, J=2.2 Hz, 3.0H), 2.08-1.48 (m, 7.2H), 1.41-1.11 (m, 13.1H), 1.01 (d, J=6.3 Hz, 1.4H), 0.92-0.86 (m, 0.4H), 0.22 (d, J=6.3 Hz, 1.7H). Isomer B (second eluting, step 3): LCMS: MS (ESI) m/z: 897.7 [M+H]⁺. ¹H NMR (400 MHz, CDCl₃, rotamers) δ 11.19 (bs, 0.5H), 8.35 (t, J=6.2 Hz, 1.0H), 8.19 (d, J=2.2 Hz, 0.6H), 8.12 (d, J=9.0 Hz, 0.6H), 8.08 (d, J=6.7 Hz, 0.6H), 7.97 (d, J=9.0 Hz, 0.4H), 7.89 (d, J=2.1 Hz, 0.4H), 7.55 (dd, J=8.6, 3.5 Hz, 1.0H), 7.46-7.35 (m, 2.0H), 7.34-7.28 (m, 1.0H), 7.25-7.18 (m, 1.2H), 7.08 (d, J=6.1 Hz, 1.2H), 6.93 (dd, J=9.0, 2.2 Hz, 0.4H), 6.89-6.81 (m, 1.4H), 6.65 (s, 0.4H), 6.57 (s, 0.6H), 6.36 (q, J=7.1 Hz, 0.4H), 4.73-4.65 (m, 0.4H), 4.26-4.19 (m, 0.6H), 3.91-3.78 (m, 2.0H), 3.77-3.67 (m, 0.7H), 3.62-3.52 (m, 0.4H), 3.24-3.17 (m, 1.1H), 3.06-2.93 (m, 2.0H), 2.92-2.90 (m, 3.1H), 2.28-1.43 (m, 16.4H), 1.39-1.10 (m, 12.2H), 1.04-0.95 (m, 1.5H), 0.92-0.84 (m, 0.3H).

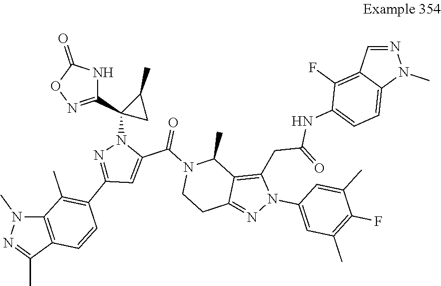

### 2-[(4S)-2-(4-fluoro-3,5-dimethyl-phenyl)-4-methyl-5-[2-[(1S,2S)-2-methyl-1-(5-oxo-4H-1,2,4-oxadiazol-3-yl)cyclopropyl]-5-(1,3,7-trimethylindazol-6-yl)pyrazole-3-carbonyl]-6,7-dihydro-4H-pyrazolo[4,3-c]pyridin-3-yl]-N-(4-fluoro-1-methyl-indazol-5-yl)acetamide

### Step 1: 2-((S)-5-(3-bromo-1-((1S,2S)-1-cyano-2-methylcyclopropyl)-1H-pyrazole-5-carbonyl)-2-(4-fluoro-3,5-dimethylphenyl)-4-methyl-4,5,6,7-tetrahydro-2H-pyrazolo[4,3-c]pyridin-3-yl)-N-(4-fluoro-1-methyl-1H-indazol-5-yl)acetamide

At room temperature under a nitrogen atmosphere, 3-bromo-1-((1S,2S)-1-cyano-2-methylcyclopropyl)-1H-pyrazole-5-carboxylic acid (350 mg, 1.30 mmol) and (S)—N-(4-fluoro-1-methyl-1H-indazol-5-yl)-2-(2-(4-fluoro-3,5-dimethylphenyl)-4-methyl-4,5,6,7-tetrahydro-2H-pyrazolo[4,3-c]pyridin-3-yl)acetamide hydrochloride (1.05 g, 1.30 mmol) were suspended in anhydrous acetonitrile (12.0 mL). [Chloro(dimethylamino)methylidene]-dimethylazanium hexafluorophosphate (473 mg, 1.68 mmol) and 1-methylimidazole (638 mg, 617 μL, 7.78 mmol) were added and the mixture was stirred at room temperature for 16 hours. The mixture was poured in water (80 mL) and extracted with dichloromethane (3×50 mL). The combined organic phases were dried over Na₂SO₄ and concentrated under reduced pressure. The residue obtained was purified by silica gel column chromatography (0-100% ethyl acetate:ethanol (3:1)/heptane) to provide the title compound LCMS: MS (ESI) m/z: 716.1, 718.1 [M+H]⁺.

### Step 2: 2-((S)-5-(3-bromo-1-((1S,2S)-1-(N-hydroxycarbamimidoyl)-2-methylcyclopropyl)-1H-pyrazole-5-carbonyl)-2-(4-fluoro-3,5-dimethylphenyl)-4-methyl-4,5,6,7-tetrahydro-2H-pyrazolo[4,3-c]pyridin-3-yl)-N-(4-fluoro-1-methyl-1H-indazol-5-yl)acetamide

At room temperature, a mixture of 2-((S)-5-(3-bromo-1-((1S,2S)-1-cyano-2-methylcyclopropyl)-1H-pyrazole-5-carbonyl)-2-(4-fluoro-3,5-dimethylphenyl)-4-methyl-4,5,6,7-tetrahydro-2H-pyrazolo[4,3-c]pyridin-3-yl)-N-(4-fluoro-1-methyl-1H-indazol-5-yl)acetamide (636 mg, 888 mol) in dimethylsulfoxide (9.54 mL) was purged with argon for 5 minutes after which an aqueous 50% solution of hydroxylamine (586 mg, 536 μL, 50% Wt, 8.88 mmol) was added. The mixture was stirred under argon atmosphere at room temperature for 20 hours. The reaction mixture was partitioned between ice cold brine (80 mL) and ethyl acetate (3×60 mL). The combined organic phases were washed using ice cold brine (2×30 mL), dried over Na₂SO₄ and concentrated under reduced pressure to provide the title compound. LCMS: MS (ESI) m/z: 749.2, 751.2 [M+H]⁺.

### Step 3: 2-((S)-5-(3-bromo-1-((1S,2S)-2-methyl-1-(5-oxo-4,5-dihydro-1,2,4-oxadiazol-3-yl)cyclopropyl)-1H-pyrazole-5-carbonyl)-2-(4-fluoro-3,5-dimethylphenyl)-4-methyl-4,5,6,7-tetrahydro-2H-pyrazolo[4,3-c]pyridin-3-yl)-N-(4-fluoro-1-methyl-1H-indazol-5-yl)acetamide

At room temperature under a nitrogen atmosphere, 2-((S)-5-(3-bromo-1-((1S,2S)-1-(N-hydroxycarbamimidoyl)-2-methylcyclopropyl)-1H-pyrazole-5-carbonyl)-2-(4-fluoro-3,5-dimethylphenyl)-4-methyl-4,5,6,7-tetrahydro-2H-pyrazolo[4,3-c]pyridin-3-yl)-N-(4-fluoro-1-methyl-1H-indazol-5-yl)acetamide (672 mg, 896 mol) and 1,8-diazabicyclo(5.4.0)undec-7-ene (512 mg, 507 μL, 3.36 mmol) were mixed in anhydrous dimethyl sulfoxide (3.36 mL). 1,1′-Carbonyldiimidazole (436 mg, 2.69 mmol) was added and the reaction mixture was stirred at room temperature for 2 hours. The mixture was poured into an aqueous 1.0 M hydrochloric acid solution (100 mL) and extracted with ethyl acetate (2×50 mL). The combined organic layers were washed with brine, dried over Na₂SO₄ and concentrated under reduced pressure. The residue obtained was purified by preparative HPLC (Reprosil-pur Basic C18-HD, 10-50% H₂O:MeCN (10 mM NH₄HCO₃ pH 9.5)) to provide the title compound. MS (ESI) m/z: 775.1, 777.1 [M+H]⁺

At room temperature under argon atmosphere, 2-((S)-5-(3-bromo-1-((1S,2S)-2-methyl-1-(5-oxo-4,5-dihydro-1,2,4-oxadiazol-3-yl)cyclopropyl)-1H-pyrazole-5-carbonyl)-2-(4-fluoro-3,5-dimethylphenyl)-4-methyl-4,5,6,7-tetrahydro-2H-pyrazolo[4,3-c]pyridin-3-yl)-N-(4-fluoro-1-methyl-1H-indazol-5-yl)acetamide (35 mg, 45 mol), 1,3,7-trimethyl-6-(4,4,5,5-tetramethyl-1,3,2-dioxaborolan-2-yl)-1H-indazole (19 mg, 68 mol) and potassium phosphate tribasic (29 mg, 0.14 mmol) were added to a mixture of 1,4-dioxane (0.36 mL) and water (90 L) under constant argon purging. After 5 minutes, tris (dibenzylideneacetone)dipalladium (6.2 mg, 6.8 mol) and bis(1,1-dimethylethyl)(methyl)phosphine tetrafluoroborate (3.4 mg, 14 mol) were added and the mixture was heated to 90° C. After 3 hours, the mixture was cooled to room temperature and purified using preparative HPLC (Reprosil-pur Basic C18-HD, 10-50% H₂O:MeCN (10 mM NH₄HCO₃ pH 9.5)) to provide the title compound. ¹H NMR (400 MHz, CDCl₃) δ 11.15 (s, 0.3H), 8.03 (s, 0.5H), 7.98-7.90 (m, 0.9H), 7.72-7.65 (m, 0.4H), 7.48 (d, J=8.3 Hz, 0.6H), 7.32-7.27 (m, 0.5H), 7.25-7.10 (m, 3.1H), 7.10-6.91 (m, 1.5H), 6.81 (d, J=9.0 Hz, 0.4H), 6.56-6.47 (m, 1.0H), 5.87 (q, J=6.5 Hz, 0.5H), 5.22 (q, J=6.6 Hz, 0.4H), 4.87 (dd, J=13.1, 5.0 Hz, 0.4H), 4.29 (s, 1.7H), 4.26-4.18 (m, 0.6H), 4.15 (s, 1.2H), 4.08 (s, 1.7H), 3.89-3.56 (m, 3.9H), 3.47-3.36 (m, 0.5H), 3.10-2.90 (m, 2.0H), 2.78 (s, 1.7H), 2.58 (s, 1.2H), 2.56-2.48 (m, 2.9H), 2.37-2.24 (m, 6.0H), 1.92 (t, J=7.1 Hz, 0.7H), 1.87-1.79 (m, 0.8H), 1.77-1.69 (m, 0.8H), 1.69-1.62 (m, 2.0), 1.52-1.35 (m, 1.0), 1.34-1.17 (m, 3.2H), 1.15-1.09 (m, 0.4H), 1.02 (d, J=6.3 Hz, 1.4H), 0.91-0.83 (m, 0.4H). LCMS: MS (ESI): 855.2 [M+H]⁺.

The following examples in table 31 were prepared according to scheme 15 using the procedures outlined in the synthesis of example 354.

TABLE 31

| | | | MS |
|---|---|---|---|
| Ex. | Structure | Name | (M+1) |
| 353 | 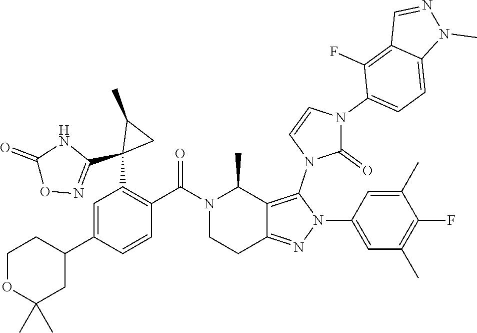 [SMILES: Cc1cc(-n2nc3c(c2-n2ccn(-c4ccc5c(cnn5C)c4F)c2=O)[C@H](C)N(C(=O)c2ccc(C4CCOC(C)(C)C4)cc2[C@@]2(c4noc(=O)[nH]4)C[C@@H]2C)CC3)cc(C)c1F] | 3-((1R,2S)-1-(5-((S)-2,2- dimethyltetrahydro-2H-pyran-4- yl)-2-((4S)-3-(3-(4-fluoro-1- methyl-1H-indazol-5-yl)-2-oxo- 2,3-dihydro-1H-imidazol-1-yl)-2- (4-fluoro-3,5-dimethylphenyl)-4- methyl-4,5,6,7-tetrahydro-2H- pyrazolo[4,3-c]pyridine-5- carbonyl)phenyl)-2- methylcyclopropyl)-1,2,4- oxadiazol-5(4H)-one or 3- ((1R,2S)-1-(5-((S)-2,2- dimethyltetrahydro-2H-pyran-4- yl)-2-((4S)-3-(3-(4-fluoro-1- methyl-1H-indazol-5-yl)-2-oxo- 2,3-dihydro-1H-imidazol-1-yl)-2- (4-fluoro-3,5-dimethylphenyl)-4- methyl-4,5,6,7-tetrahydro-2H- pyrazolo[4,3-c]pyridine-5- carbonyl)phenyl)-2- methylcyclopropyl)-1,2,4- oxadiazol-5(4H)-one | 844.0 |
| 354 | 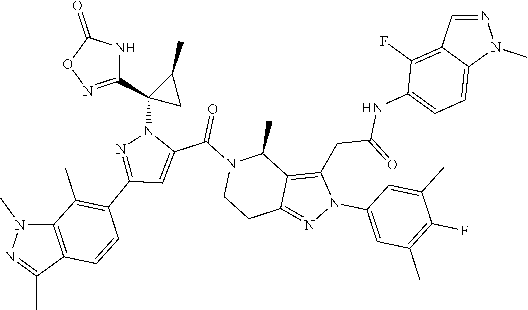 | 2-[(4S)-2-(4-fluoro-3,5-dimethyl- phenyl)-4-methyl-5-[2-[(1S,2S)- 2-methyl-1-(5-oxo-4H-1,2,4- oxadiazol-3-yl)cyclopropyl]-5- (1,3,7-trimethylindazol-6- yl)pyrazole-3-carbonyl]-6,7- dihydro-4H-pyrazolo[4,3- c]pyridin-3-yl]-N-(4-fluoro-1- methyl-indazol-5-yl)acetamide | 855.2 |
| 355 | 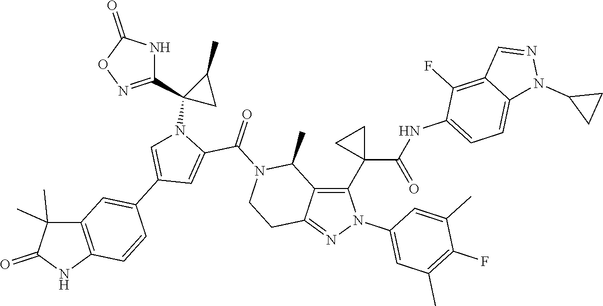 [SMILES: Cc1cc(-n2nc3c(c2C2(C(=O)Nc4ccc5c(cnn5C5CC5)c4F)CC2)[C@H](C)N(C(=O)c2cc(-c4ccc5c(c4)C(C)(C)C(=O)N5)cn2[C@@]2(c4noc(=O)[nH]4)C[C@@H]2C)CC3)cc(C)c1F] | N-(1-cyclopropyl-4-fluoro- indazol-5-yl)-1-[(4S)-5-[5-(3,3- dimethyl-2-oxo-indolin-5-yl)-2- [(1S,2S)-2-methyl-1-(5-oxo-4H- 1,2,4-oxadiazol-3- yl)cyclopropyl]pyrazole-3- carbonyl]-2-(4-fluoro-3,5- dimethyl-phenyl)-4-methyl-6,7- dihydro-4H-pyrazolo[4,3- c]pyridin-3- yl]cyclopropanecarboxamide | |
| 356 | 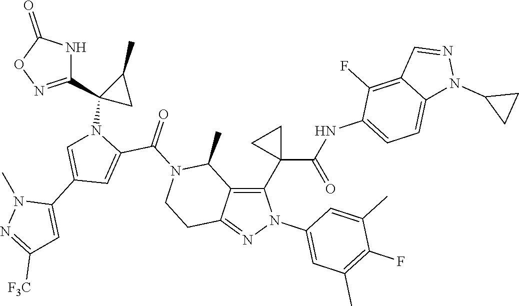 [SMILES: Cc1cc(-n2nc3c(c2C2(C(=O)Nc4ccc5c(cnn5C5CC5)c4F)CC2)[C@H](C)N(C(=O)c2cc(-c4cc(C(F)(F)F)nn4C)cn2[C@@]2(c4noc(=O)[nH]4)C[C@@H]2C)CC3)cc(C)c1F] | N-(1-cyclopropyl-4-fluoro- indazol-5-yl)-1-[(4S)-2-(4-fluoro- 3,5-dimethyl-phenyl)-4-methyl-5- [2-[(1S,2S)-2-methyl-1-(5-oxo- 4H-1,2,4-oxadiazol-3- yl)cyclopropyl]-5-[1-methyl-3- (trifluoromethyl)pyrazol-5- yl]pyrazole-3-carbonyl]-6,7- dihydro-4H-pyrazolo[4,3- c]pyridin-3- yl]cyclopropanecarboxamide | 897.4 |
| 357 | 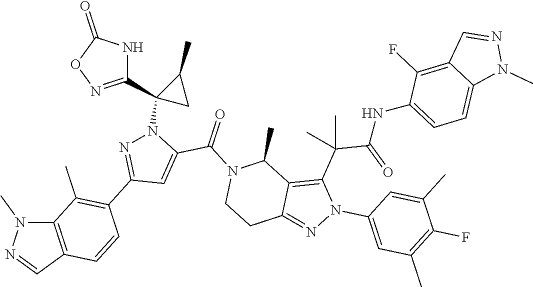 | 1-[(4S)-5-[5-(1,7- dimethylindazol-6-yl)-2-[(1S,2S)- 2-methyl-1-(5-oxo-4H-1,2,4- oxadiazol-3- yl)cyclopropyl]pyrazole-3- carbonyl]-2-(4-fluoro-3,5- dimethyl-phenyl)-4-methyl-6,7- dihydro-4H-pyrazolo[4,3- c]pyridin-3-yl]-N-(4-fluoro-1- methyl-indazol-5- yl)cyclopropanecarboxamide | 867.2 |
| 358 | 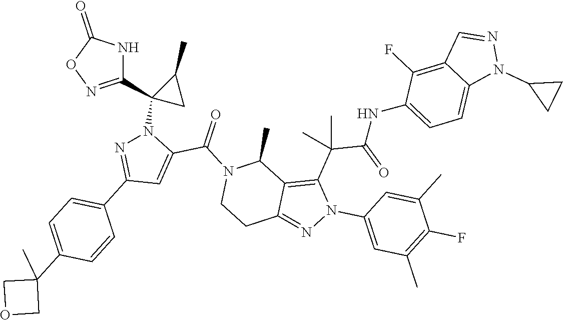 [SMILES: Cc1cc(-n2nc3c(c2C(C)(C)C(=O)Nc2ccc4c(cnn4C4CC4)c2F)[C@H](C)N(C(=O)c2cc(-c4ccc(C5(C)COC5)cc4)nn2[C@@]2(c4noc(=O)[nH]4)C[C@@H]2C)CC3)cc(C)c1F] | N-(1-cyclopropyl-4-fluoro- indazol-5-yl)-1-[(4S)-2-(4-fluoro- 3,5-dimethyl-phenyl)-4-methyl-5- [5-[4-(3-methyloxetan-3- yl)phenyl]-2-[(1S,2S)-2-methyl- 1-(5-oxo-4H-1,2,4-oxadiazol-3- yl)cyclopropyl]pyrazole-3- carbonyl]-6,7-dihydro-4H- pyrazolo[4,3-c]pyridin-3- yl]cyclopropanecarboxamide | |
| 359 | 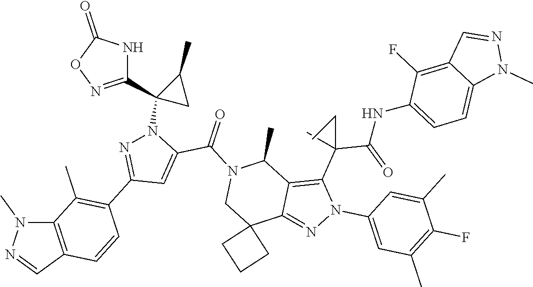 | N-(1-cyclopropyl-4-fluoro- indazol-5-yl)-1-[(4S)-5-[5-(1,7- dimethylindazol-6-yl)-2-[(1S,2S)- 2-methyl-1-(5-oxo-4H-1,2,4- oxadiazol-3- yl)cyclopropyl]pyrazole-3- carbonyl]-2-(4-fluoro-3,5- dimethyl-phenyl)-4-methyl-6,7- dihydro-4H-pyrazolo[4,3- c]pyridin-3- yl]cyclopropanecarboxamide | 907.2 |
| 360 | 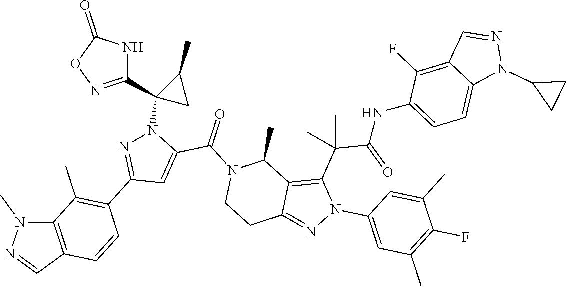 [SMILES: Cc1cc(-n2nc3c(c2C(C)(C)C(=O)Nc2ccc4c(cnn4C4CC4)c2F)[C@H](C)N(C(=O)c2cc(-c4ccc5cnn(C)c5c4C)nn2[C@@]2(c4noc(=O)[nH]4)C[C@@H]2C)CC3)cc(C)c1F] | N-(1-cyclopropyl-4-fluoro- indazol-5-yl)-1-[(4S)-5-[5-(1,7- dimethylindazol-6-yl)-2-[(1S,2S)- 2-methyl-1-(5-oxo-4H-1,2,4- oxadiazol-3- yl)cyclopropyl]pyrazole-3- carbonyl]-2-(4-fluoro-3,5- dimethyl-phenyl)-4-methyl-6,7- dihydro-4H-pyrazolo[4,3- c]pyridin-3- yl]cyclopropanecarboxamide | 893.2 |
| 361 | 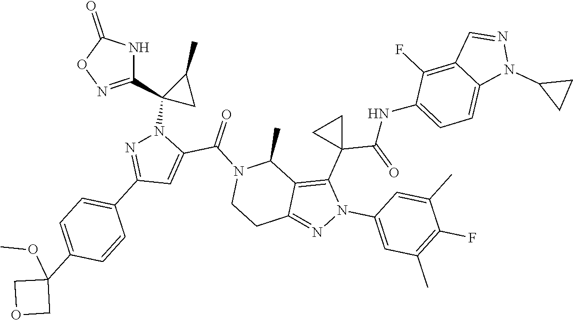 [SMILES: COC1(c2ccc(-c3cc(C(=O)N4CCc5nn(-c6cc(C)c(F)c(C)c6)c(C6(C(=O)Nc7ccc8c(cnn8C8CC8)c7F)CC6)c5[C@@H]4C)n([C@@]4(c5noc(=O)[nH]5)C[C@@H]4C)n3)cc2)COC1] | N-(1-cyclopropyl-4-fluoro- indazol-5-yl)-1-[(4S)-2-(4-fluoro- 3,5-dimethyl-phenyl)-5-[5-[4-(1- methoxycyclobutyl)phenyl]-2- [(1S,2S)-2-methyl-1-(5-oxo-4H- 1,2,4-oxadiazol-3- yl)cyclopropyl]pyrazole-3- carbonyl]-4-methyl-6,7-dihydro- 4H-pyrazolo[4,3-c]pyridin-3- yl]cyclopropanecarboxamide | 909.4 |
| 362 | 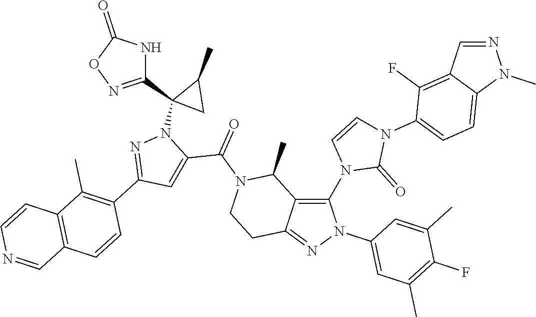 [SMILES: Cc1cc(-n2nc3c(c2-n2ccn(-c4ccc5c(cnn5C)c4F)c2=O)[C@H](C)N(C(=O)c2cc(-c4ccc5cnccc5c4C)nn2[C@@]2(c4noc(=O)[nH]4)C[C@@H]2C)CC3)cc(C)c1F] | 3-[(1S,2S)-1-[5-[(4S)-2-(4-fluoro- 3,5-dimethyl-phenyl)-3-[3-(4- fluoro-1-methyl-indazol-5-yl)-2- oxo-imidazol-1-yl]-4-methyl-6,7- dihydro-4H-pyrazolo[4,3- c]pyridine-5-carbonyl]-3-(5- methyl-6-isoquinolyl)pyrazol-1- yl]-2-methyl-cyclopropyl]-4H- 1,2,4-oxadiazol-5-one | 863.4 |
| 363 | 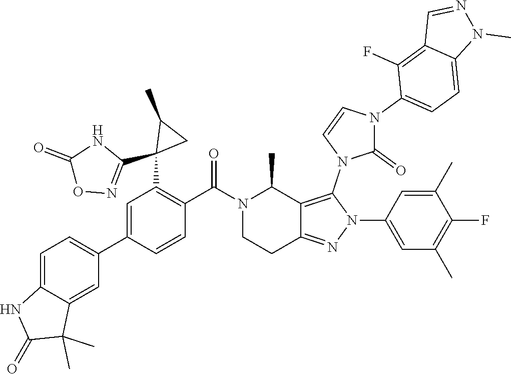 [SMILES: Cc1cc(-n2nc3c(c2-n2ccn(-c4ccc5c(cnn5C)c4F)c2=O)[C@H](C)N(C(=O)c2ccc(-c4ccc5c(c4)C(C)(C)C(=O)N5)cc2[C@@]2(c4noc(=O)[nH]4)C[C@@H]2C)CC3)cc(C)c1F] | 5-[4-[(4S)-2-(4-fluoro-3,5- dimethyl-phenyl)-3-[3-(4-fluoro- 1-methyl-indazol-2-ium-5-yl)-2- oxo-imidazol-1-yl]-4-methyl-6,7- dihydro-4H-pyrazolo[4,3- c]pyridin-1-ium-5-carbonyl]-3- [(1R,2S)-1-(5-hydroxy-1,2,4- oxadiazole-2,4-diium-3-yl)-2- methyl-cyclopropyl]phenyl]-3,3- dimethyl-indolin-2-one | 891.7 |
| 400 | 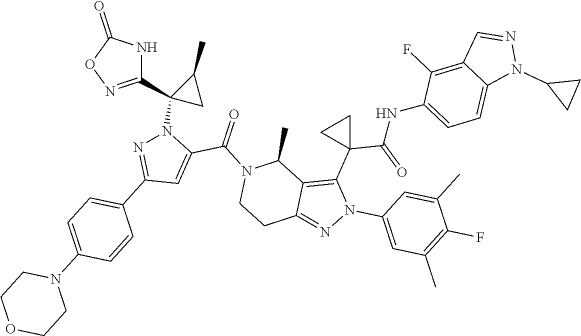 [SMILES: Cc1cc(-n2nc3c(c2C2(C(=O)Nc4ccc5c(cnn5C5CC5)c4F)CC2)[C@H](C)N(C(=O)c2cc(-c4ccc(N5CCOCC5)cc4)nn2[C@@]2(c4noc(=O)[nH]4)C[C@@H]2C)CC3)cc(C)c1F] | N-(1-cyclopropyl-4-fluoro-1H- indazol-5-yl)-1-((S)-2-(4-fluoro- 3,5-dimethylphenyl)-4-methyl-5- (1-((1S,2S)-2-methyl-1-(5-oxo- 4,5-dihydro-1,2,4-oxadiazol-3- yl)cyclopropyl)-3-(4- morpholinophenyl)-1H-pyrazole- 5-carbonyl)-4,5,6,7-tetrahydro- 2H-pyrazolo[4,3-c]pyridin-3- yl)cyclopropane-1-carboxamide | 910.4 |
| 414 | 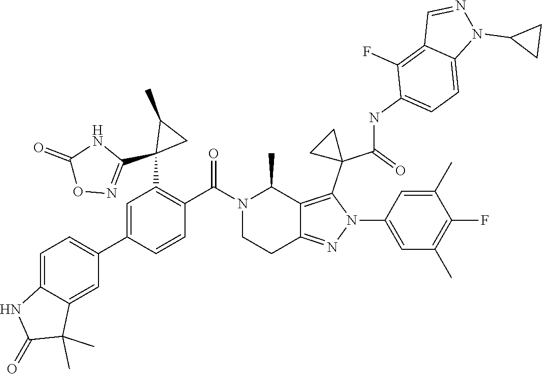 [SMILES: Cc1cc(-n2nc3c(c2C2(C(=O)Nc4ccc5c(cnn5C5CC5)c4F)CC2)[C@H](C)N(C(=O)c2ccc(-c4ccc5c(c4)C(C)(C)C(=O)N5)cc2[C@@]2(c4noc(=O)[nH]4)C[C@@H]2C)CC3)cc(C)c1F] | N-(1-cyclopropyl-4-fluoro-1H- indazol-5-yl)-1-((S)-5-(4-(3,3- dimethyl-2-oxoindolin-5-yl)-2- ((1R,2S)-2-methyl-1-(5-oxo-4,5- dihydro-1,2,4-oxadiazol-3- yl)cyclopropyl)benzoyl)-2-(4- fluoro-3,5-dimethylphenyl)-4- methyl-4,5,6,7-tetrahydro-2H- pyrazolo[4,3-c]pyridin-3- yl)cyclopropane-1-carboxamide | 918.4 |
| 415 | 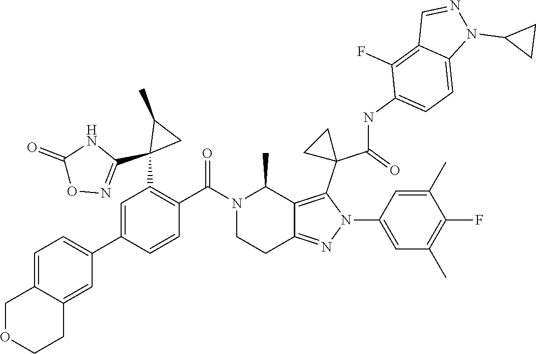 [SMILES: Cc1cc(-n2nc3c(c2C2(C(=O)Nc4ccc5c(cnn5C5CC5)c4F)CC2)[C@H](C)N(C(=O)c2ccc(-c4ccc5c(c4)CCOC5)cc2[C@@]2(c4noc(=O)[nH]4)C[C@@H]2C)CC3)cc(C)c1F] | N-(1-cyclopropyl-4-fluoro-1H- indazol-5-yl)-1-((S)-2-(4-fluoro- 3,5-dimethylphenyl)-5-(4- (isochroman-6-yl)-2-((1R,2S)-2- methyl-1-(5-oxo-4,5-dihydro- 1,2,4-oxadiazol-3- yl)cyclopropyl)benzoyl)-4- methyl-4,5,6,7-tetrahydro-2H- pyrazolo[4,3-c]pyridin-3- yl)cyclopropane-1-carboxamide | 891.2 |
| 416 | 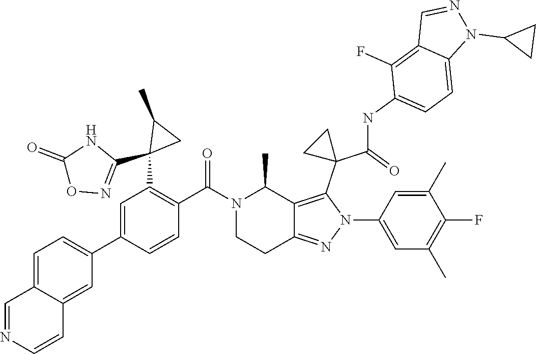 [SMILES: Cc1cc(-n2nc3c(c2C2(C(=O)Nc4ccc5c(cnn5C5CC5)c4F)CC2)[C@H](C)N(C(=O)c2ccc(-c4ccc5cnccc5c4)cc2[C@@]2(c4noc(=O)[nH]4)C[C@@H]2C)CC3)cc(C)c1F] | N-(1-cyclopropyl-4-fluoro-1H- indazol-5-yl)-1-((S)-2-(4-fluoro- 3,5-dimethylphenyl)-5-(4- (isoquinolin-6-yl)-2-((1R,2S)-2- methyl-1-(5-oxo-4,5-dihydro- 1,2,4-oxadiazol-3- yl)cyclopropyl)benzoyl)-4- methyl-4,5,6,7-tetrahydro-2H- pyrazolo[4,3-c]pyridin-3- yl)cyclopropane-1-carboxamide | 886.4 |
| 423 | 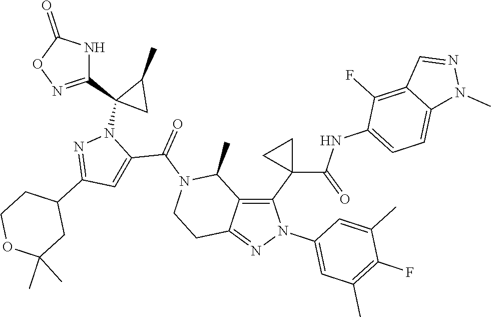 [SMILES: Cc1cc(-n2nc3c(c2C2(C(=O)Nc4ccc5c(cnn5C)c4F)CC2)[C@H](C)N(C(=O)c2cc(C4CCOC(C)(C)C4)nn2[C@@]2(c4noc(=O)[nH]4)C[C@@H]2C)CC3)cc(C)c1F] | 1-((S)-5-(3-((S)-2,2- dimethyltetrahydro-2H-pyran-4- yl)-1-((1S,2S)-2-methyl-1-(5- oxo-4,5-dihydro-1,2,4-oxadiazol- 3-yl)cyclopropyl)-1H-pyrazole-5- carbonyl)-2-(4-fluoro-3,5- dimethylphenyl)-4-methyl- 4,5,6,7-tetrahydro-2H- pyrazolo[4,3-c]pyridin-3-yl)-N- (4-fluoro-1-methyl-1H-indazol-5- yl)cyclopropane-1-carboxamide OR 1-((S)-5-(3-((S)-2,2- dimethyltetrahydro-2H-pyran-4- yl)-1-((1S,2S)-2-methyl-1-(5- oxo-4,5-dihydro-1,2,4-oxadiazol- 3-yl)cyclopropyl)-1H-pyrazole-5- carbonyl)-2-(4-fluoro-3,5- dimethylphenyl)-4-methyl- 4,5,6,7-tetrahydro-2H- pyrazolo[4,3-c]pyridin-3-yl)-N- (4-fluoro-1-methyl-1H-indazol-5- yl)cyclopropane-1-carboxamide | 835.0 |
| 424 | 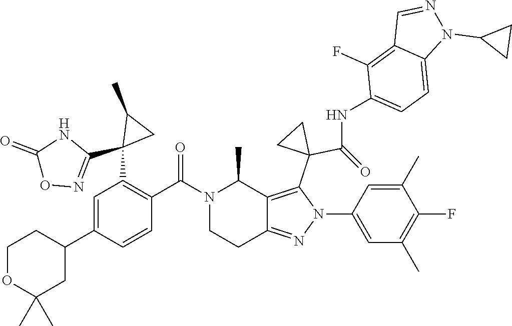 [SMILES: Cc1cc(-n2nc3c(c2C2(C(=O)Nc4ccc5c(cnn5C5CC5)c4F)CC2)[C@H](C)N(C(=O)c2ccc(C4CCOC(C)(C)C4)cc2[C@@]2(c4noc(=O)[nH]4)C[C@@H]2C)CC3)cc(C)c1F] | N-(1-cyclopropyl-4-fluoro-1H- indazol-5-yl)-1-((S)-5-(4-((S)-2,2- dimethyltetrahydro-2H-pyran-4- yl)-2-((1R,2S)-2-methyl-1-(5- oxo-4,5-dihydro-1,2,4-oxadiazol- 3-yl)cyclopropyl)benzoyl)-2-(4- fluoro-3,5-dimethylphenyl)-4- methyl-4,5,6,7-tetrahydro-2H- pyrazolo[4,3-c]pyridin-3- yl)cyclopropane-1-carboxamide AND N-(1-cyclopropyl-4-fluoro- 1H-indazol-5-yl)-1-((S)-5-(4-((S)- 2,2-dimethyltetrahydro-2H- pyran-4-yl)-2-((1R,2S)-2-methyl- 1-(5-oxo-4,5-dihydro-1,2,4- oxadiazol-3- yl)cyclopropyl)benzoyl)-2-(4- fluoro-3,5-dimethylphenyl)-4- methyl-4,5,6,7-tetrahydro-2H- pyrazolo[4,3-c]pyridin-3- yl)cyclopropane-1-carboxamide | 871.3 |
| 425 A | 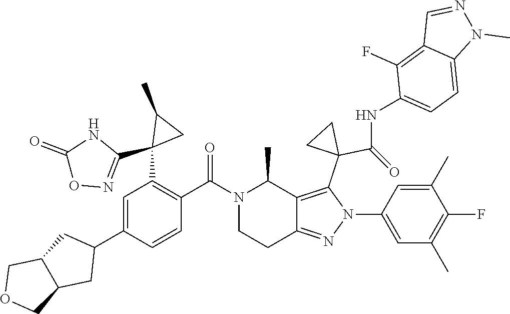 [SMILES: Cc1cc(-n2nc3c(c2C2(C(=O)Nc4ccc5c(cnn5C)c4F)CC2)[C@H](C)N(C(=O)c2ccc(C4C[C@H]5COC[C@@H]5C4)cc2[C@@]2(c4noc(=O)[nH]4)C[C@@H]2C)CC3)cc(C)c1F] OR 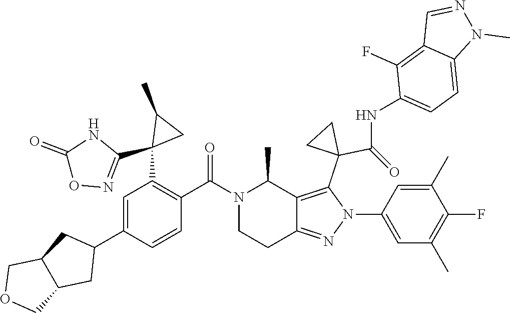 [SMILES: Cc1cc(-n2nc3c(c2C2(C(=O)Nc4ccc5c(cnn5C)c4F)CC2)[C@H](C)N(C(=O)c2ccc(C4C[C@@H]5COC[C@H]5C4)cc2[C@@]2(c4noc(=O)[nH]4)C[C@@H]2C)CC3)cc(C)c1F] | N-(4-fluoro-1-methyl-1H- indazol-5-yl)-1-((S)-2-(4-fluoro- 3,5-dimethylphenyl)-5-(4- ((3aS,6aS)-hexahydro-1H- cyclopenta[c]furan-5-yl)-2- ((1R,2S)-2-methyl-1-(5-oxo-4,5- dihydro-1,2,4-oxadiazol-3- yl)cyclopropyl)benzoyl)-4- methyl-4,5,6,7-tetrahydro-2H- pyrazolo[4,3-c]pyridin-3- yl)cyclopropane-1-carboxamide OR N-(4-fluoro-1-methyl-1H- indazol-5-yl)-1-((S)-2-(4-fluoro- 3,5-dimethylphenyl)-5-(4- ((3aR,6aR)-hexahydro-1H- cyclopenta[c]furan-5-yl)-2- ((1R,2S)-2-methyl-1-(5-oxo-4,5- dihydro-1,2,4-oxadiazol-3- yl)cyclopropyl)benzoyl)-4- methyl-4,5,6,7-tetrahydro-2H- pyrazolo[4,3-c]pyridin-3- yl)cyclopropane-1-carboxamide | 843.6 |
| 425 B | 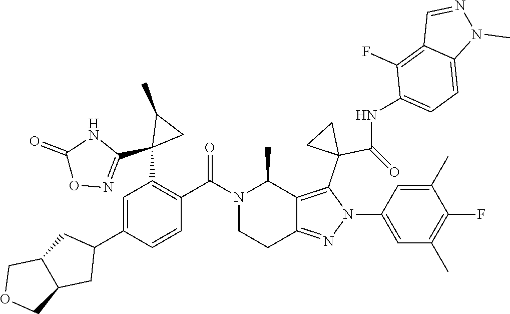 [SMILES: Cc1cc(-n2nc3c(c2C2(C(=O)Nc4ccc5c(cnn5C)c4F)CC2)[C@H](C)N(C(=O)c2ccc(C4C[C@H]5COC[C@@H]5C4)cc2[C@@]2(c4noc(=O)[nH]4)C[C@@H]2C)CC3)cc(C)c1F] OR 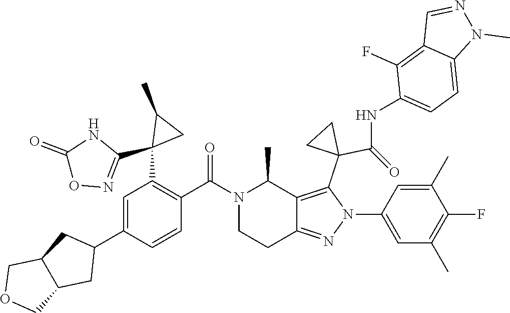 [SMILES: Cc1cc(-n2nc3c(c2C2(C(=O)Nc4ccc5c(cnn5C)c4F)CC2)[C@H](C)N(C(=O)c2ccc(C4C[C@@H]5COC[C@H]5C4)cc2[C@@]2(c4noc(=O)[nH]4)C[C@@H]2C)CC3)cc(C)c1F] | N-(4-fluoro-1-methyl-1H- indazol-5-yl)-1-((S)-2-(4-fluoro- 3,5-dimethylphenyl)-5-(4- ((3aS,6aS)-hexahydro-1H- cyclopenta[c]furan-5-yl)-2- ((1R,2S)-2-methyl-1-(5-oxo-4,5- dihydro-1,2,4-oxadiazol-3- yl)cyclopropyl)benzoyl)-4- methyl-4,5,6,7-tetrahydro-2H- pyrazolo[4,3-c]pyridin-3- yl)cyclopropane-1-carboxamide OR N-(4-fluoro-1-methyl-1H- indazol-5-yl)-1-((S)-2-(4-fluoro- 3,5-dimethylphenyl)-5-(4- ((3aR,6aR)-hexahydro-1H- cyclopenta[c]furan-5-yl)-2- ((1R,2S)-2-methyl-1-(5-oxo-4,5- dihydro-1,2,4-oxadiazol-3- yl)cyclopropyl)benzoyl)-4- methyl-4,5,6,7-tetrahydro-2H- pyrazolo[4,3-c]pyridin-3- yl)cyclopropane-1-carboxamide | |
| 426 A | 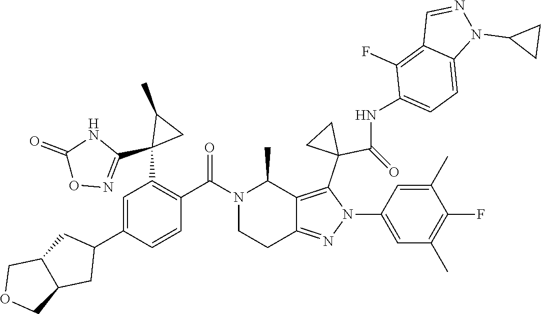 [SMILES: Cc1cc(-n2nc3c(c2C2(C(=O)Nc4ccc5c(cnn5C5CC5)c4F)CC2)[C@H](C)N(C(=O)c2ccc(C4C[C@H]5COC[C@@H]5C4)cc2[C@@]2(c4noc(=O)[nH]4)C[C@@H]2C)CC3)cc(C)c1F] OR 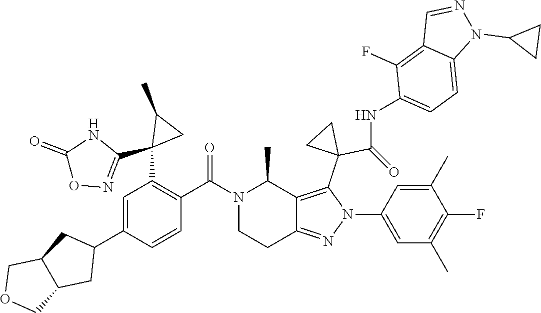 [SMILES: Cc1cc(-n2nc3c(c2C2(C(=O)Nc4ccc5c(cnn5C5CC5)c4F)CC2)[C@H](C)N(C(=O)c2ccc(C4C[C@@H]5COC[C@H]5C4)cc2[C@@]2(c4noc(=O)[nH]4)C[C@@H]2C)CC3)cc(C)c1F] | N-(1-cyclopropyl-4-fluoro-1H- indazol-5-yl)-1-((S)-2-(4-fluoro- 3,5-dimethylphenyl)-5-(4- ((3aS,6aS)-hexahydro-1H- cyclopenta[c]furan-5-yl)-2- ((1R,2S)-2-methyl-1-(5-oxo-4,5- dihydro-1,2,4-oxadiazol-3- yl)cyclopropyl)benzoyl)-4- methyl-4,5,6,7-tetrahydro-2H- pyrazolo[4,3-c]pyridin-3- yl)cyclopropane-1-carboxamide OR N-(1-cyclopropyl-4-fluoro- 1H-indazol-5-yl)-1 -((S)-2-(4- fluoro-3,5-dimethylphenyl)-5-(4- ((3aR,6aR)-hexahydro-1H- cyclopenta[c]furan-5-yl)-2- ((1R,2S)-2-methyl-1-(5-oxo-4,5- dihydro-1,2,4-oxadiazol-3- yl)cyclopropyl)benzoyl)-4- methyl-4,5,6,7-tetrahydro-2H- pyrazolo[4,3-c]pyridin-3- yl)cyclopropane-1-carboxamide | 869.6 |
| 426 B | 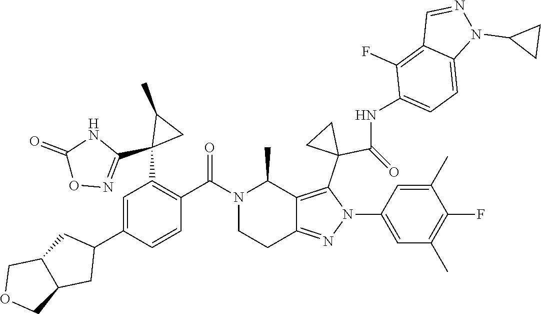 [SMILES: Cc1cc(-n2nc3c(c2C2(C(=O)Nc4ccc5c(cnn5C5CC5)c4F)CC2)[C@H](C)N(C(=O)c2ccc(C4C[C@H]5COC[C@@H]5C4)cc2[C@@]2(c4noc(=O)[nH]4)C[C@@H]2C)CC3)cc(C)c1F] OR 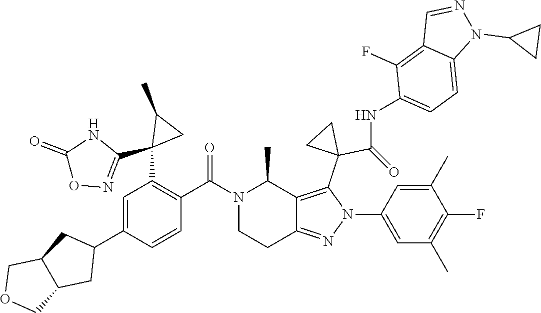 [SMILES: Cc1cc(-n2nc3c(c2C2(C(=O)Nc4ccc5c(cnn5C5CC5)c4F)CC2)[C@H](C)N(C(=O)c2ccc(C4C[C@@H]5COC[C@H]5C4)cc2[C@@]2(c4noc(=O)[nH]4)C[C@@H]2C)CC3)cc(C)c1F] | N-(1-cyclopropyl-4-fluoro-1H- indazol-5-yl)-1-((S)-2-(4-fluoro- 3,5-dimethylphenyl)-5-(4- ((3aS,6aS)-hexahydro-1H- cyclopenta[c]furan-5-yl)-2- ((1R,2S)-2-methyl-1-(5-oxo-4,5- dihydro-1,2,4-oxadiazol-3- yl)cyclopropyl)benzoyl)-4- methyl-4,5,6,7-tetrahydro-2H- pyrazolo[4,3-c]pyridin-3- yl)cyclopropane-1-carboxamide OR N-(1-cyclopropyl-4-fluoro- 1H-indazol-5-yl)-1 -((S)-2-(4- fluoro-3,5-dimethylphenyl)-5-(4- ((3aR,6aR)-hexahydro-1H- cyclopenta[c]furan-5-yl)-2- ((1R,2S)-2-methyl-1-(5-oxo-4,5- dihydro-1,2,4-oxadiazol-3- yl)cyclopropyl)benzoyl)-4- methyl-4,5,6,7-tetrahydro-2H- pyrazolo[4,3-c]pyridin-3- yl)cyclopropane-1-carboxamide | 869.6 |
| 427 A | 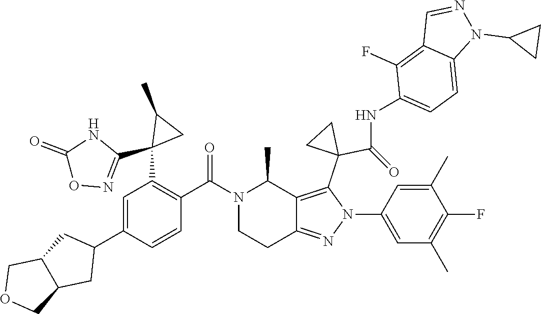 [SMILES: Cc1cc(-n2nc3c(c2C2(C(=O)Nc4ccc5c(cnn5C5CC5)c4F)CC2)[C@H](C)N(C(=O)c2ccc(C4C[C@H]5COC[C@@H]5C4)cc2[C@@]2(c4noc(=O)[nH]4)C[C@@H]2C)CC3)cc(C)c1F] OR 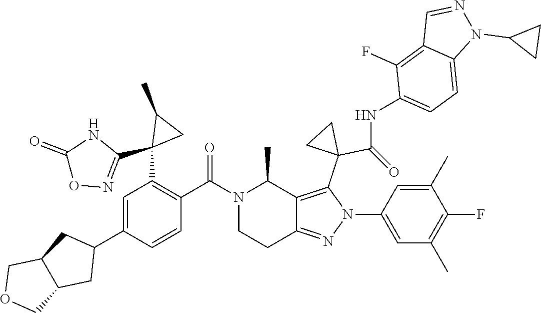 [SMILES: Cc1cc(-n2nc3c(c2C2(C(=O)Nc4ccc5c(cnn5C5CC5)c4F)CC2)[C@H](C)N(C(=O)c2ccc(C4C[C@@H]5COC[C@H]5C4)cc2[C@@]2(c4noc(=O)[nH]4)C[C@@H]2C)CC3)cc(C)c1F] | N-(1-cyclopropyl-4-fluoro-1H- indazol-5-yl)-1-((S)-5-(4-((S)-2,2- dimethyltetrahydro-2H-pyran-4- yl)-2-((1R,2S)-2-methyl-1-(5- oxo-4,5-dihydro-1,2,4-oxadiazol- 3-yl)cyclopropyl)benzoyl)-2-(4- fluoro-3,5-dimethylphenyl)-4- methyl-4,5,6,7-tetrahydro-2H- pyrazolo[4,3-c]pyridin-3- yl)cyclopropane-1-carboxamide OR N-(1-cyclopropyl-4-fluoro- 1H-indazol-5-yl)-1-((S)-5-(4-((S)- 2,2-dimethyltetrahydro-2H- pyran-4-yl)-2-((1R,2S)-2-methyl- 1-(5-oxo-4,5-dihydro-1,2,4- oxadiazol-3- yl)cyclopropyl)benzoyl)-2-(4- fluoro-3,5-dimethylphenyl)-4- methyl-4,5,6,7-tetrahydro-2H- pyrazolo[4,3-c]pyridin-3- yl)cyclopropane-1-carboxamide | 871.4 |
| 427 B | 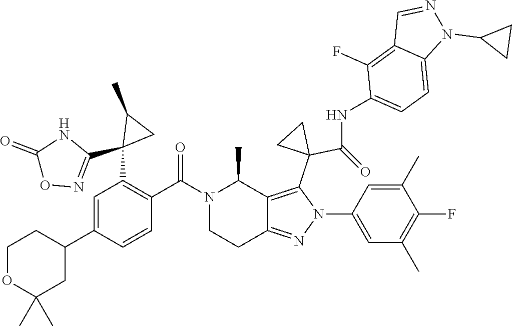 [SMILES: Cc1cc(-n2nc3c(c2C2(C(=O)Nc4ccc5c(cnn5C5CC5)c4F)CC2)[C@H](C)N(C(=O)c2ccc(C4CCOC(C)(C)C4)cc2[C@@]2(c4noc(=O)[nH]4)C[C@@H]2C)CC3)cc(C)c1F] OR 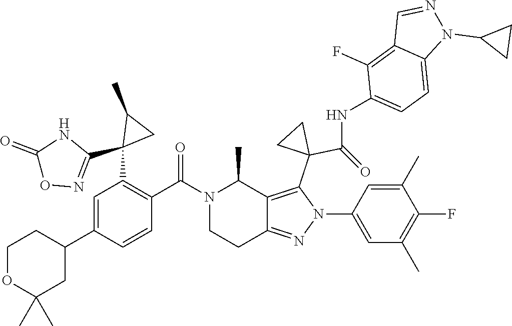 [SMILES: Cc1cc(-n2nc3c(c2C2(C(=O)Nc4ccc5c(cnn5C5CC5)c4F)CC2)[C@H](C)N(C(=O)c2ccc(C4CCOC(C)(C)C4)cc2[C@@]2(c4noc(=O)[nH]4)C[C@@H]2C)CC3)cc(C)c1F] | N-(1-cyclopropyl-4-fluoro-1H- indazol-5-yl)-1-((S)-5-(4-((S)-2,2- dimethyltetrahydro-2H-pyran-4- yl)-2-((1R,2S)-2-methyl-1-(5- oxo-4,5-dihydro-1,2,4-oxadiazol- 3-yl)cyclopropyl)benzoyl)-2-(4- fluoro-3,5-dimethylphenyl)-4- methyl-4,5,6, 7-tetrahydro-2H- pyrazolo[4,3-c]pyridin-3- yl)cyclopropane-1-carboxamide OR N-(1-cyclopropyl-4-fluoro- 1H-indazol-5-yl)-1-((S)-5-(4-((S)- 2,2-dimethyltetrahydro-2H- pyran-4-yl)-2-((1R,2S)-2-methyl- 1-(5-oxo-4,5-dihydro-1,2,4- oxadiazol-3- yl)cyclopropyl)benzoyl)-2-(4- fluoro-3,5-dimethylphenyl)-4- methyl-4,5,6,7-tetrahydro-2H- pyrazolo[4,3-c]pyridin-3- yl)cyclopropane-1-carboxamide | 871.4 |

### Biological Assays

To measure the effect of test articles on cAMP activation, CHO-K1 cells that overexpress human GLP-1 receptor (DiscoverX, 93-0300C2) were seeded in assay buffer containing 1×HBSS, 10 mM HEPES (pH 7.4), 0.5 mM IBMX, and 0.1% BSA. Test articles in a range of dose titrations were then added to the cells and incubated at 37° C. with 5% CO₂ for 30 minutes. Subsequently, cAMP levels were determined by using a Gs HiRange detection kit (Revvity, cat #62AM6PEC) under manufacturer recommended conditions. HTRF signals were measured on an EnVision plate reader. The ratiometric data were normalized as percentage of cAMP response elicited by a saturating concentration of human GLP-1 and analyzed using a 4-parameter logistic fit equation to derive EC₅₀ and % maximal response values.

| | GLP-1R | | GLP-1R | | GLP-1R |
|---|---|---|---|---|---|
| Ex. No. | EC50 (nM) | Ex. No. | EC50 (nM) | Ex. No. | EC50 (nM) |
| 1 | 0.11 | 45 | 0.29 | 96 | 0.22 |
| 2 | 0.17 | 46 | 0.52 | 97 | 0.34 |
| 3 | 0.12 | 47 | 0.43 | 98 | 0.06 |
| 4 | 0.08 | 48 | 0.15 | 99 | 0.07 |
| 5 | Not available | 49 | 0.08 | 100 | 0.09 |
| 6 | >126 | 50 | 0.48 | 101 | 0.14 |
| 7 | 0.39 | 51 | 0.53 | 102 | 0.11 |
| 8 | 0.62 | 52 | 0.25 | 103 | 0.23 |
| 9 | 0.58 | 53 | 0.53 | 104 | 0.05 |
| 10 | 0.90 | 54 | 0.25 | 105 | 0.32 |
| 11 | 0.83 | 55 | 0.12 | 106 | 0.71 |
| 12 | 0.13 | 56 | 0.12 | 107 | 0.40 |
| 13 | 0.38 | 57 | 0.33 | 108 | 0.44 |
| 14 | 0.06 | 58 | 0.56 | 109A | 11.41 |
| 15 | 0.12 | 59 | 6.13 | 109B | 83.33 |
| 16 | 0.27 | 60 | 0.88 | 110 | 0.16 |
| 17 | 0.56 | 61 | 1.23 | 111 | 1.86 |
| 18 | 0.18 | 62 | 0.73 | 112 | 0.15 |
| 19 | 0.04 | 63 | 0.44 | 113 | 1.78 |
| 20A | 3.11 | 64 | 0.19 | 114A | 0.25 |
| 20B | 2.56 | 65 | 0.84 | 114B | 0.09 |
| 21A | 0.88 | 66 | 0.50 | 115B | 0.22 |
| 21B | 0.88 | 67 | 0.70 | 116B | 0.11 |
| 22A | 0.64 | 68 | 1.38 | 117B | 0.20 |
| 22B | 0.91 | 69 | 0.20 | 118B | 0.20 |
| 23A | 0.88 | 70 | 0.48 | 119B | 0.27 |
| 23B | 0.60 | 71 | 0.24 | 120B | 0.30 |
| 24A | 1.10 | 72 | 0.35 | 121B | 0.12 |
| 24B | 126.60 | 73 | 0.07 | 122B | 0.49 |
| 25A | 0.77 | 74 | 0.04 | 123B | 0.12 |
| 25B | 1.45 | 75 | 0.12 | 124 | 0.38 |
| 26 | 0.21 | 76 | 0.20 | 125 | 0.24 |
| 27 | 0.26 | 77 | 0.23 | 126 | 0.25 |
| 28 | 0.21 | 78 | 0.03 | 127 | 0.45 |
| 29 | 0.15 | 79 | 0.44 | 128 | 0.46 |
| 30 | 0.81 | 80 | 0.09154 | 129 | 0.61 |
| 31 | 0.42 | 81 | 0.13 | 130 | 0.29 |
| 32 | 0.21 | 82 | 0.06 | 131 | 0.26 |
| 33 | 0.51 | 83 | 0.03 | 132 | 0.73 |
| 34 | 0.38 | 84 | 0.11 | 133 | 0.41 |
| 35 | 0.55 | 85 | 0.05 | 134 | 0.19 |
| 36 | 0.37 | 86 | 0.12 | 135 | 0.98 |
| 37 | 0.43 | 87 | 0.04 | 136 | 0.68 |
| 38 | 0.51 | 88 | 0.06 | 137A | 0.60 |
| 38 | 0.74 | 89 | 0.33 | 138 | 0.31 |
| 39 | 0.31 | 90 | 0.17 | 139 | 0.25 |
| 40 | 0.38 | 91 | 0.99 | 140 | 0.17 |
| 41 | 0.27 | 92 | 0.35 | 141 | 0.86 |
| 42 | 0.51 | 93 | 0.14 | 142 | 0.48 |
| 43 | 0.33 | 94 | 0.04 | 143 | 0.83 |
| 44 | 0.87 | 95 | 0.07 | 144 | 0.45 |
| 145 | 0.70 | 193 | 1.033 | 240 | 0.026 |
| 146 | 0.52 | 194 | 0.355 | 241 | 0.020 |
| 147 | 0.88 | 195 | 0.135 | 242 | 0.027 |
| 148 | 0.07 | 196 | 0.325 | 243 | 0.072 |
| 149 | 0.13 | 197 | 0.067 | 244 | 0.183 |
| 150 | 0.15 | 198 | 0.351 | 245 | 0.128 |
| 151 | 0.04 | 199 | 0.133 | 246 | 0.102 |
| 152 | 0.06 | 200 | 0.058 | 247 | 0.078 |
| 153 | 0.08 | 201 | 0.039 | 248 | 0.037 |
| 154 | 0.11 | 202 | 0.065 | 250 | 0.048 |
| 155 | 1.196 | 203 | 0.497 | 251 | 0.083 |
| 156B | 0.62 | 204 | 0.230 | 252 | 0.187 |
| 157A | 0.08 | 205 | 0.030 | 253 | 0.303 |
| 157B | 0.79 | 206 | 0.179 | 254 | 0.434 |
| 158A | 0.29 | 207 | 0.023 | 255 | 1.054 |
| 158B | 0.73 | 208 | 0.027 | 260 | 0.037 |
| 159A | 0.21 | 209 | 0.082 | 262 | 0.040 |
| 159B | 3.89 | 210 | 0.023 | 263 | 0.088 |
| 161 | 1.72 | 211 | 0.060 | 264 | 0.092 |
| 162 | 0.17 | 212 | 0.012 | 265 | 0.054 |
| 163 | 0.78 | 213 | 0.148 | 265 | 0.031 |
| 164 | 2.92 | 214 | 0.115 | 266 | 0.017 |
| 165 | 11.75 | 215 A | 0.298 | 268 A | 0.063 |
| | | or B | | | |
| 166 | 7.62 | 216A | 0.197 | 268 B | 0.050 |
| 167 | 126.60 | 216B | 1.135 | 271 A | 0.023 |
| 168 | 0.08 | 217 | 0.071 | 271 B | 0.031 |
| 169 | 0.55 | 218 | 0.055 | 273 A | 0.266 |
| 170 | 0.090 | 219 | 0.140 | 273 B | 0.054 |
| 171 | 0.120 | 220 | 0.469 | 275 A | 0.079 |
| 172 | 0.210 | 221 | 0.056 | 275 B | 0.360 |
| 173 | 0.257 | 222 | 0.193 | 277 A | 0.139 |
| 174 | 0.058 | 223 | 0.052 | 277 B | 0.057 |
| 175 | 0.062 | 224 | 0.036 | 279 A | 0.130 |
| 176 | 0.263 | 225 A | 0.220 | 279 B | 0.185 |
| 177 | 0.131 | 225 B | 1.820 | 281 | 0.109 |
| 178 | 0.129 | 226 A | 2.085 | 282 | 0.168 |
| 179 | 0.030 | 226 B | 0.298 | 283 | 0.078 |
| 180 | 0.026 | 227 | 0.014 | 284 | 0.060 |
| 181 | 0.216 | 228 | 0.219 | 285 | 0.033 |
| 182 | 0.154 | 229 | 0.046 | 286 | 0.022 |
| 183 | 0.360 | 230 | 0.035 | 287 | 0.055 |
| 184 | 3.400 | 231 | 0.148 | 288 | 0.042 |
| 185 | 0.158 | 232 | 0.571 | 289 | 0.097 |
| 186 | 0.049 | 233 | 0.601 | 290 | 0.056 |
| 187 | 0.118 | 234 | 0.028 | 291 | 0.024 |
| 188 | 0.042 | 235 | 0.022 | 292 | 0.538 |
| 189 | 0.158 | 236 | 0.112 | 293 | 0.066 |
| 190 | 0.179 | 237 | 0.190 | 294 | 0.059 |
| 191 | 0.294 | 238 | 0.147 | 295 | 0.345 |
| 192 | 0.786 | 239 | 0.016 | 296 | 0.133 |
| 297 | 0.201 | 342 | 0.131 | 392 | 0.060 |
| 298 | 0.142 | 343 | 0.227 | 393 | 0.070 |
| 299 | 0.070 | 344 | 0.515 | 394 | 0.073 |
| 300 A | 0.068 | 345 | 0.305 | 395 | 0.075 |
| 300 B | 0.135 | 346 | 0.489 | 396 | 0.077 |
| 305 A | 0.023 | 347 | 0.079 | 397 | 0.102 |
| 305 B | 0.022 | 348 | 0.092 | 398 | 0.106 |
| 307 A | 6.638 | 349 | 0.322 | 399 | 0.120 |
| 307 B | 0.114 | 350 | 0.108 | 400 | 0.123 |
| 308 A | 0.772 | 351 | 0.042 | 401 | 0.208 |
| 308 B | 0.041 | 352 | 0.096 | 402 | 0.218 |
| 309 A | 1.294 | 353 | 0.112 | 403 | 0.059 |
| 309 B | 0.043 | 354 | 0.038 | 404 | 0.061 |
| 310 A | 6.521 | 355 | 0.042 | 405 | 0.040 |
| 310 B | 0.145 | 356 | 0.070 | 406 A | 0.070 |
| 311 A | 0.156 | 357 | 0.077 | 406 B | 4.134 |
| 311 B | 0.386 | 358 | 0.079 | 407 | 0.045 |
| 312 | 0.309 | 359 | 0.081 | 408 B | 0.026 |
| 313 | 0.113 | 360 | 0.085 | 408 A | 0.456 |
| 314 | 0.430 | 361 | 0.092 | 409 | 0.099 |
| 315 | 0.163 | 362 | 0.142 | 410 | 0.340 |
| 316 | 0.070 | 363 | 0.119 | 411 | 0.070 |
| 317 A | 0.120 | 364 | 0.032 | 412 | 0.146 |
| 317 B | 0.583 | 365 | 0.053 | 413 | 0.299 |
| 318 | 0.544 | 366 | 0.058 | 414 | 0.028 |
| 319 | 0.046 | 367 | 0.059 | 415 | 0.059 |
| 320 A | 0.403 | 368 | 0.066 | 416 | 0.217 |
| 320 B | 6.818 | 369 | 0.067 | 417 | 0.163 |
| 322 | 0.068 | 370 | 0.098 | 418 | 0.067 |
| 323 | 0.167 | 371 | 0.118 | 419 | 0.061 |
| 324 A | 2.376 | 372 | 0.126 | 420 | 0.231 |
| 324 B | >126.6 | 373 | 0.148 | 421 | 0.035 |
| 325 | 0.441 | 374 | 0.150 | 422 | 0.366 |
| 326 | 0.619 | 375 | 0.156 | 423 | 0.027 |
| 327 | 0.026 | 376 | 0.163 | 424 | 0.131 |
| 328 | 0.045 | 377 | 0.042 | 425A | 0.039 |
| 330 | 0.121 | 378 | 0.049 | 425B | 0.116 |
| 331 A | 1.509 | 379 | 0.063 | | |
| 331 B | 0.193 | 380 | 0.124 | | |
| 332 | 0.339 | 381 | 0.147 | | |
| 333 A | 0.147 | 382 | 0.155 | | |
| 333 B | 0.046 | 383 | 0.168 | | |
| 334 A | 3.841 | 384 | 0.270 | | |
| 334 B | 0.215 | 385 | 0.030 | | |
| 335 | 0.039 | 386 | 0.031 | | |
| 336 | 0.053 | 387 | 0.031 | | |
| 337 | 2.135 | 388 | 0.032 | | |
| 338 | 0.163 | 389 A | 0.046 | | |
| 339 | 0.049 | 389 B | 0.038 | | |
| 340 | 0.274 | 390 | 0.038 | | |
| 341 | 0.581 | 391 | 0.059 | | |

### Pharmacokinetic Experiments

Plasma pharmacokinetic parameters for mean residence time (MRT) were determined in cynomolgus monkeys from IV administration studies. Two monkeys typically weighing 7 to 10 kg were fasted overnight prior to dosing. Compounds were prepared for IV dosing by addition to a vehicle, depending on the dose used. For a typical preparation, 0.2 mg per kg (IV) of the test compound was added to a vehicle comprised of 10% ethanol, 70% polyethylene glycol (PEG400), and 20% water. IV formulation was administered to two monkeys via the saphenous/cephalic vein using a butterfly set followed by a saline flush. Blood was collected from the saphenous or femoral vein, typically at pre-dose, and 0.03, 0.13, 0.25, 0.5, 1, 2, 4, 6, and 24 hours post-dose.

Blood samples were collected in K2EDTA tubes, stored on ice, and centrifuged. Plasma was stored at −70° C. until analysis. Plasma samples were extracted using protein precipitation and analyzed by liquid chromatography separation followed by mass spec detection (LC-MS/MS), using a standard curve for each compound. Plasma pharmacokinetic parameters were calculated by non-compartmental methods. Pharmacokinetic properties of compounds of the disclosure and a prior art compound are provided in the following table:

| Ex No. | MRT (h) |
|---|---|
| 1 | 5.059 |
| 3 | 2.639 |
| 4 | 1.482 |
| 15 | 2.474 |
| 26 | 2.688^{a} |
| 27 | 4.89 |
| 29 | 4.925 |
| 31 | 3.579 |
| 38 | 4.668 |
| 41 | 12.78 |
| 47 | 9.716 |
| 60 | 8.506 |
| 63 | 5.747 |
| 71 | 11.14 |
| 72 | 8.243 |
| 73 | 5.692 |
| 75 | 10.08 |
| 79 | 11.76 |
| 103 | 8.431 |
| 149 | 3.838 |
| 168 | 1.948 |
| 169 | 6.445 |
| 176 | 2.373 |
| 187 | 3.163 |
| 189 | 4.852 |
| 190 | 1.253 |
| 191 | 1.532 |
| 202 | 2.942 |
| 208 | 1.274 |
| 260 | 1.698 |
| 281 | 6.565 |
| 347 | 4.267 |
| 115B | 6.817 |
| 116B | 3.729 |
| 157A | 2.117 |
| 158A | 6.03 |
| 159A | 5.173 |
| 225A | 13 |
| 226B | 3.764 |
| 268B | 2.96 |
| orforglipron | 4.855^{b} |
| orforglipron | 8.94^{c} |

^{a}For the compound of Example 26, 0.1 mg per kg (IV) of the test compound was added to a vehicle comprised of 10% ethanol, 70% polyethylene glycol (PEG400), and 20% water.
^{b}For this orforglipron experiment, 0.1 mg per kg (IV) of the test compound was added to a vehicle comprised of 10% ethanol, 70% polyethylene glycol (PEG400), and 20% water
^{c}For this orforglipron experiment, 0.1 mg per kg (IV) of the test compound was added to a vehicle comprised of 10% DMSO, 40% polyethylene glycol (PEG400), and 50% water.

Mean residence time (MRT) is independent of dose in classical linear pharmacokinetics. However, MRT may change with dose if any elimination or distribution is concentration-dependent (nonlinear kinetics). In the present case, neither elimination nor distribution are expected to be concentration-dependent at doses of 0.1 mg per kg and 0.2 mg per kg.

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

What is claimed is:

1. A compound of Formula (I) 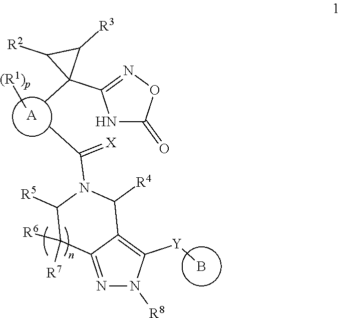
or a pharmaceutically acceptable salt thereof,
wherein:
R¹ is halogen, CN, OH, aminoalkyl, hydroxyalkyl, diaminoalkyl, dihydroxyalkyl, C₁-C₆ alkyl, C₁-C₆ heteroalkyl, C₃-C₈ cycloalkyl, 3-10 membered heterocycloalkyl, —C≡C-(3-10 membered heterocycloalkyl), —C≡C—(C₃-C₈ cycloalkyl), or -phenyl-(C₃-C₈ cycloalkyl), wherein R¹ is substituted with 0 to 5 substituents independently selected from halogen, CN, hydroxyl, C₁-C₆ alkyl, 3-10 membered heterocycloalkyl or C₃-C₈ cycloalkyl, wherein the C₁-C₆ alkyl, 3-10 membered heterocycloalkyl or C₃-C₈ cycloalkyl is substituted with one or more halogen atoms, C₁-C₂ alkyl, or C₁-C₆ alkyloxy;
R² is H or methyl;
R³ is H or methyl;
R⁴ is hydrogen or C₁-C₆ alkyl;
R⁵ is hydrogen or C₁-C₆ alkyl; or
R⁴ and R⁵, together with the atoms which they are attached, or R⁴ and R⁶, together with the atom which they are attached, form a bridged C₇-C₈ heterocyclic ring fused to the pyrazole;
each R⁶ is independently H, halo, or C₁-C₆ alkyl and each R⁷ is independently H, halo, or C₁-C₆ alkyl, or
R⁶ and R⁷ together with the atoms to which they are attached, form a C₃-C₆ cycloalkyl ring or a 3-6 membered heterocycloalkyl ring, wherein the C₃-C₆ cycloalkyl ring or the 3-6 membered heterocycloalkyl ring is substituted with 0 or 1 instances of R⁹, provided that if there are two R⁶ and R⁷ then only one set forms a ring and the other R⁶ and R⁷ is each independently H or C₁-C₆ alkyl;
R⁹ is H, C₁-C₆ alkyl, —SO₂—(C₁-C₆ alkyl), —((C₁-C₆ alkylenyl)O)ₜ(C₁-C₆ alkyl), —(C₁-C₆ alkylenyl)(C₃-C₈cycloalkyl), —(C)(O)(C₁-C₆ alkyl), —(CH₂)aryl, C₃-C₈ cycloalkyl, —(CH₂)(C₃-C₈ cycloalkyl), C(O)C₁-C₆ alkyl, —((C₁-C₆ alkylenyl)O)ₜ(C₁-C₆ alkyl), or S(O)₂(C₁-C₆ alkyl) wherein one or more of the hydrogen atoms of the C₁-C₆ alkyl may be substituted with fluoro or hydroxyl, and 0 or 1 of the carbons of the C₁-C₆ alkyl form a cyclopropyl ring, and wherein t is 1, 2, 3, 4 or 5;
n is 1 or 2;
p is 1, 2, 3, 4 or 5;
R⁴, R⁵, R⁶ and R⁷ are each independently substituted with 0 to 2 substituents independently selected from halogen, NH₂, aminoalkyl, diaminoalkyl, C₁-C₆ alkyl, and C₁-C₆ alkyl substituted with one or more halogen atoms, C₁₋₆ alkoxy, hydroxy, oxo, CONR^{w1}R^{w2}, SO₂N R^{w3}R^{w4}, SO₂R^{w5}, N(R^{w6})COR^{w7}, NR^{w8}SO₂R^{w9}, wherein R^{w1},R^{w2}, R^{w3}, R^{w4}, R^{w5}, R^{w6}, R^{w7},R^{w8}, and R^{w9}, are each individually selected from H and C₁-C₆ alkyl;
A is 5-10 membered heteroaryl or C₆-C₁₀ aryl;
X is O, S, or NR^{XX} wherein R^{XX} is H or C₁-C₃ alkyl, wherein C₁-C₃ alkyl is substituted with 0, 1, or 2 substituents selected from halogen and C₃-C₆ cycloalkyl; 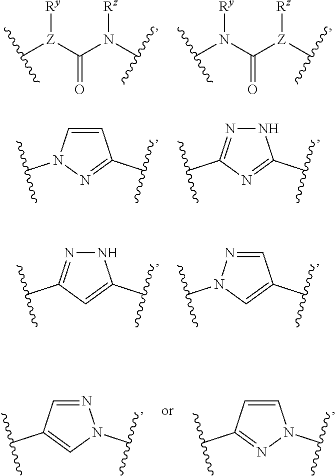
wherein the left [symbol] on Y is bound to the pyrazole moiety and the right [symbol] on Y is bound to B,
Z is C, CR^{x} or N;
R^{x} is H, halo, hydroxy, C₁-C₆ alkyl, C₃-C₆ cycloalkyl, C₁-C₆ alkoxy, or —O(C₃-C₆ cycloalkyl);
R^{y}, and R^{z}, together with the atoms which they are attached and optionally R^{x}, form a 5-8 membered heterocycloalkyl ring or a 5-6 membered heteroaryl; or
R^{y}, and R^{z} are each independently H, halo, C₁-C₆ alkyl or C₃-C₆ cycloalkyl; or
R^{x} and R^{y}, together with the carbon to which they are attached, form a spiro C₃-C₆ cycloalkyl ring and R^{z} is H, halo, or C₁-C₆ alkyl; or
R^{x} and R^{z}, together with the carbon to which they are attached, form a spiro C₃-C₆ cycloalkyl ring and R^{y} is H, halo, or C₁-C₆ alkyl; and
B is C₃-C₈ cycloalkyl, 5-13 membered heterocycloalkyl, aryl or heteroaryl, and B is substituted by 0, 1, 2, or 3 substituents, each independently selected from C₁-C₃ alkyl, halogen, hydroxyl, heteroalkyl, 5-9 membered heterocycloalkyl, —O(C₃-C₆ cycloalkyl), C₃-C₆ cycloalkyl, 5-9 membered heteroaryl, 5-9 membered aryl, —O(C₆-C₁₀ aryl), and phosphorus atom substituted with oxo and/or C₁-C₃ alkyl, wherein one or more of the hydrogen atoms of the C₁-C₃ alkyl or heteroalkyl may be substituted with deuterium or fluoro, and wherein the 5-9 membered heterocycloalkyl, 5-9 membered heteroaryl or 5-9 membered aryl is substituted with 0, 1, 2, or 3 substituents independently selected from —C₁-C₃ alkyl, —C₁-C₃ haloalkyl, —C₃-C₆ cycloalkyl, and oxo;
R⁸ is C₁-C₆ alkyl, 5-10 membered heterocycloalkyl, heteroaryl, or aryl, and R⁸ is substituted by 0, 1, 2, or 3 substituents, each independently selected from C₁-C₃ alkyl, C₃-C₆ cycloalkyl, —O(C₃-C₆ cycloalkyl), phenyl, halogen, —SO₂CH₃ and oxo, wherein one or more of the hydrogen atoms of the C₁-C₃ alkyl or C₃-C₆ cycloalkyl may be substituted with fluoro or chloro; or
R⁸ is R^{8a}R^{8b} and R^{8a} is 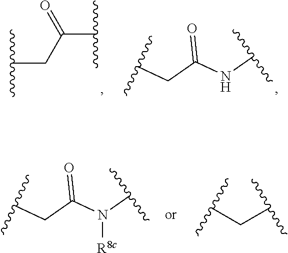
and R^{8b} is aryl, heteroaryl, 4-10 membered heterocycloalkyl, C₃-C₁₀ cycloalkyl, wherein R^{8b} is substituted by 0, 1, 2 or 3 substituents, each independently selected from C₁-C₃ alkyl, halogen, and oxo, wherein one or more of the hydrogen atoms of the C₁-C₃ alkyl may be substituted with fluoro or chloro; wherein if R^{8a} is then R^{8b} is 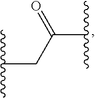 [SMILES: *CC(*)=O]
then R^{8b} is heteroaryl or 4-10 membered heterocycloalkyl with at least one nitrogen, and R^{8a} forms a bond with the nitrogen of the heteroaryl or 4-10 membered heterocycloalkyl;
wherein R^{8c}, when present, is C₁-C₆ alkyl, substituted with 0, 1, 2, or 3 substituents independently selected from halogen, C₃-C₆ cycloalkyl, phenyl or 3-8 membered heterocycloalkyl;
wherein at least one of (a)-(h) is true:
(a) R⁸ is other than optionally substituted C₆₋₁₀ aryl and optionally substituted or 5 to 10 membered heteroaryl;
(b) Y is other than 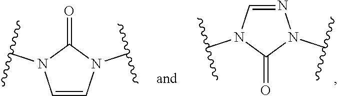 [SMILES: *n1ccn(*)c1=O ; *n1cnn(*)c1=O]
wherein the left [symbol] on Y is bound to the pyrazole moiety and the right [symbol] on Y is bound to B, and
(c) R¹ is other than optionally substituted 3 to 12 membered heterocycloalkyl and 5 to 10 membered heteroaryl;
(d) n is 1 and R⁶ is C₁-C₆ alkyl and R⁷ is C₁-C₆ alkyl, or
(e) n is 1 and R⁶ and R⁷ together with the atom to which they are attached, form a C₃-C₆ cycloalkyl ring or a 3-6 membered heterocycloalkyl ring;
(f) n is 2; and
(g) Y is other than 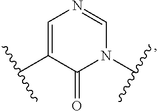 [SMILES: *c1cncn(*)c1=O]
or Y is 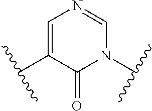 [SMILES: *c1cncn(*)c1=O]
and B is 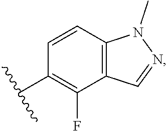 [SMILES: *c1ccc2c(cnn2C)c1F]
wherein the left [symbol] on Y is bound to the pyrazole moiety and the right [symbol] on Y is bound to B; and
(h) Y is other than 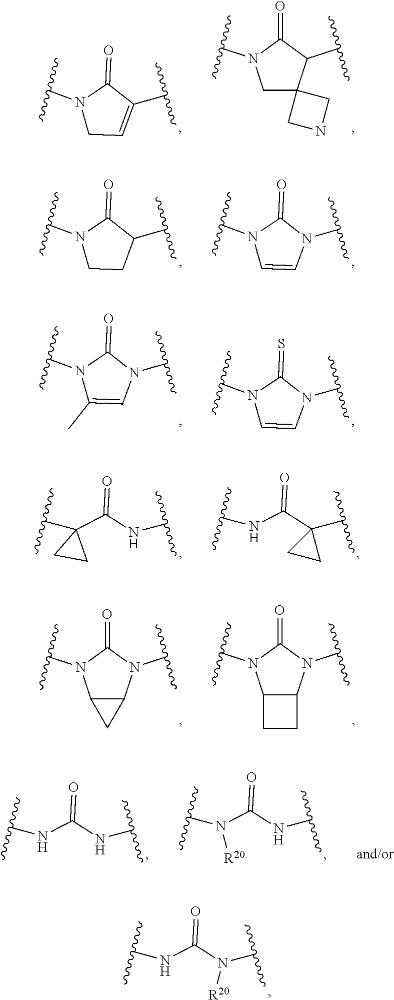
wherein the left [symbol] on Y is bound to the pyrazole moiety and the right [symbol] on Y is bound to B and R²⁰ is selected from C₁-C₃ alkyl or cyclopropyl.

2. The compound of claim 1, or a pharmaceutically acceptable salt thereof, wherein A is 6-9 membered heteroaryl or C₆-C₉ aryl.

3. The compound of claim 2, or a pharmaceutically acceptable salt thereof, wherein A is

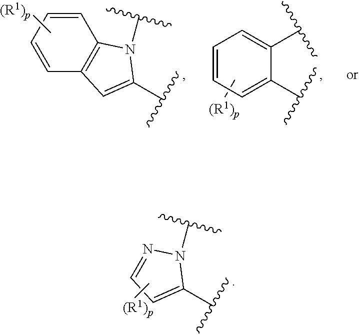

4. The compound of claim 3, or a pharmaceutically acceptable salt thereof, wherein

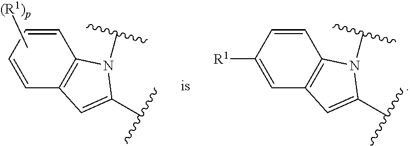

5. The compound of claim 3, or a pharmaceutically acceptable salt thereof, wherein A is

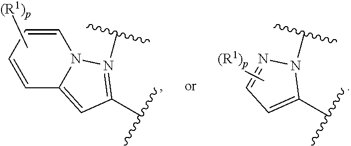

6. The compound of claim 3, or a pharmaceutically acceptable salt thereof, wherein

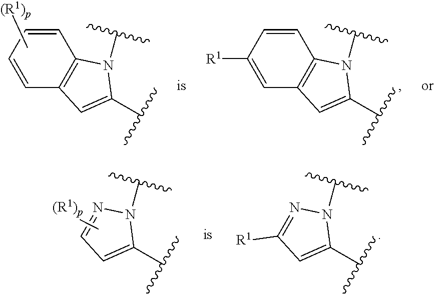

7. The compound of claim 3, or a pharmaceutically acceptable salt thereof, wherein

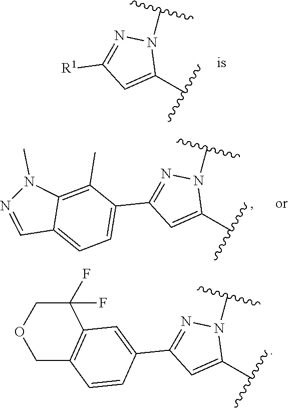

8. The compound of any one of claims 1 to 7, or a pharmaceutically acceptable salt thereof, wherein R¹ is 6-10 membered heterocycloalkyl or C₃-C₈ cycloalkyl, wherein R¹ is substituted with 0 to 5 substituents independently selected from a halogen, C₁₋₆ alkyl, C₁₋₆ alkyl substituted with one or more halogen atoms, and C₁₋₆ alkoxy.

9. The compound of claim 8, or a pharmaceutically acceptable salt thereof, wherein R¹ is 6-10 membered heterocycloalkyl, wherein R¹ is substituted with 0 to 2 substituents independently selected from C₁₋₆ alkyl.

10. The compound of claim 8, or a pharmaceutically acceptable salt thereof, wherein R¹ is 6-10 membered heterocycloalkyl, wherein R¹ is substituted with two methyl groups.

11. The compound of claim 8, or a pharmaceutically acceptable salt thereof, wherein R¹ is 6-10 membered heterocycloalkyl containing one or two oxygen atoms as ring members, wherein R¹ is substituted with 0 to 3 substituents independently selected from OH, C₁-C₆ alkyl, or C₁-C₆ alkyloxy.

12. The compound of claim 8, or a pharmaceutically acceptable salt thereof, wherein R¹ is C₃-C₈ cycloalkyl, wherein R¹ is substituted with 0 to 2 substituents independently selected from hydroxyl and C₁-C₆ alkoxy.

13. The compound of claim 8, or a pharmaceutically acceptable salt thereof, wherein R¹ is 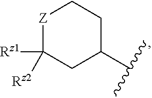
Z is O or CH(OCH₃); R^{z1} is H or C₁-C₆ alkyl and R^{z2} is H or C₁-C₆ alkyl.

14. The compound of claim 8, or a pharmaceutically acceptable salt thereof, wherein R¹ is 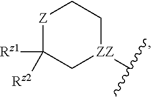
Z is O or CH(OCH₃); R^{z1} is H, halogen, or C₁-C₆ alkyl and R^{z2} is H, halogen or C₁-C₆ alkyl, and
ZZ is N.

15. The compound of claim 8, or a pharmaceutically acceptable salt thereof, wherein R¹ is tetrahydropyranyl, wherein R¹ is substituted with 0 or 2 methyl groups.

16. The compound of claim 8, or a pharmaceutically acceptable salt thereof, wherein R¹ is

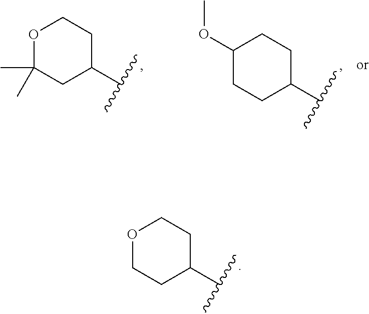

17. The compound of claim 8, or a pharmaceutically acceptable salt thereof, wherein R¹ is 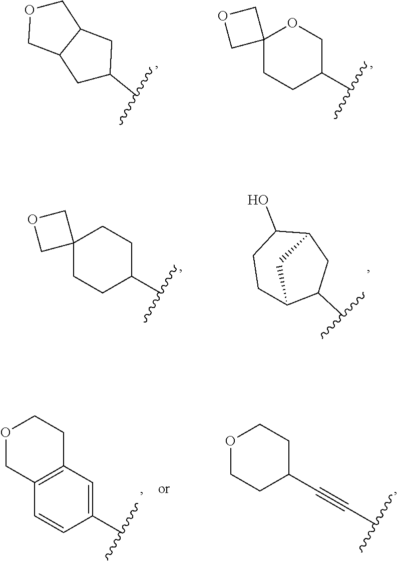 [SMILES: *C1CC2COCC2C1 ; *C1CCC2(COC2)OC1 ; *C1CCC2(CC1)COC2 ; *C1C[C@H]2C[C@@H]1CCC2O ; *c1ccc2c(c1)CCOC2 ; *C#CC1CCOCC1]
and R¹ is substituted with 0 to 3 substituents independently selected from halogen, C₁-C₆ alkyl, C₁-C₆ alkyl substituted with one or more halogen atoms, and C₁-C₆ alkoxy.

18. The compound of claim 8, or a pharmaceutically acceptable salt thereof, wherein R¹ is

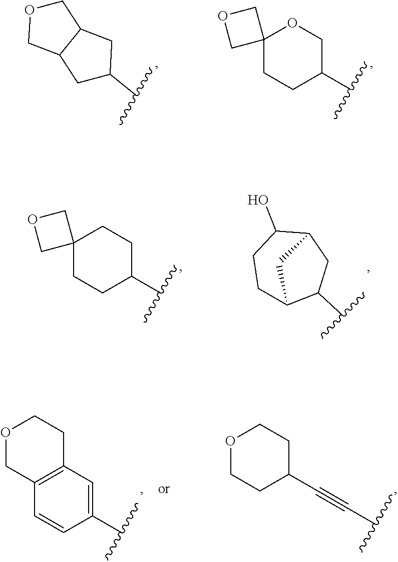 [SMILES: *C1CC2COCC2C1 ; *C1CCC2(COC2)OC1 ; *C1CCC2(CC1)COC2 ; *C1C[C@H]2C[C@@H]1CCC2O ; *c1ccc2c(c1)CCOC2 ; *C#CC1CCOCC1]

19. The compound of claim 8, or a pharmaceutically acceptable salt thereof, wherein R¹ is

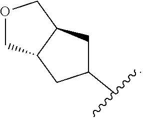 [SMILES: *C1C[C@@H]2COC[C@H]2C1]

20. The compound of claim 8, or a pharmaceutically acceptable salt thereof, wherein R¹ is

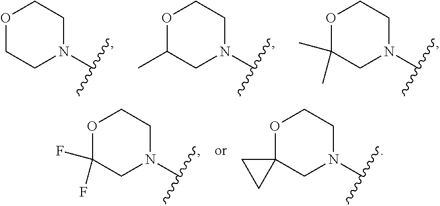

21. The compound of claim 8, or a pharmaceutically acceptable salt thereof, wherein R¹ is

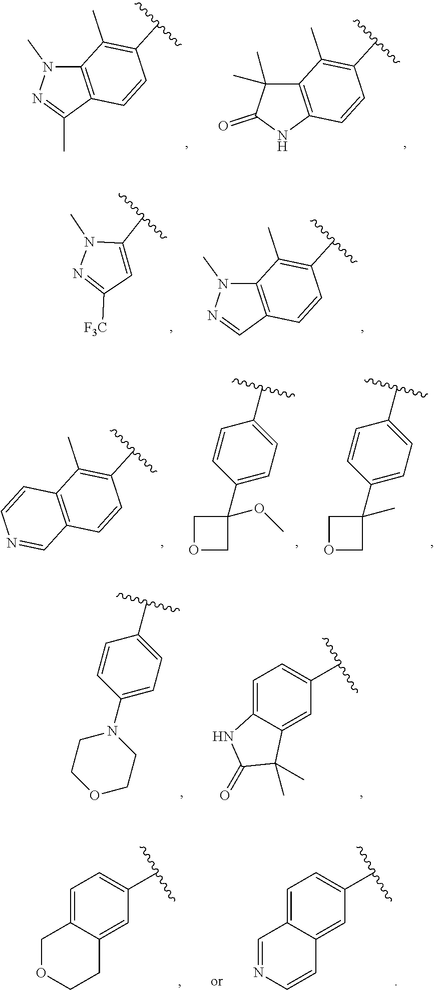

22. The compound of any one of claims 1 to 21, or a pharmaceutically acceptable salt thereof, wherein R² is H and R³ is H.

23. The compound of any one of claims 1 to 22, or a pharmaceutically acceptable salt thereof, wherein R² is H and R³ is methyl.

24. The compound of any one of claims 1 to 23, or a pharmaceutically acceptable salt thereof, wherein the compound of Formula I has the Formula Ia:

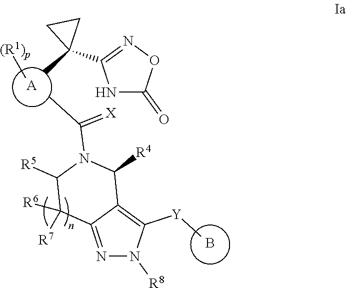

25. The compound of any one of claims 1 to 24, or a pharmaceutically acceptable salt thereof, wherein
R⁴ is hydrogen, methyl, or ethyl;
R⁵ is hydrogen or methyl;
R⁴ and R⁵, together with the atom which they are attached, or R⁴ and R⁶, together with the atom which they are attached, form a bridged C₇₋₈ heterocyclic ring fused to the pyrazole; and
R⁶ is H, halo, or methyl, and R⁷ is H, halo, or methyl, or
R⁶ and R⁷ together with the atom to which they are attached, form a cyclopropyl ring or a cyclobutyl ring provided that if there are two R⁶ and R⁷ then only one set forms a ring and the other R⁶ and R⁷ is each independently H or methyl.

26. The compound of any one of claims 1 to 25, or a pharmaceutically acceptable salt thereof, wherein R⁴ is methyl.

27. The compound of any one of claims 1 to 26, or a pharmaceutically acceptable salt thereof, wherein R⁵ is H.

28. The compound of any one of claims 1 to 25, or a pharmaceutically acceptable salt thereof, wherein R⁴ is methyl and R⁵ is H.

29. The compound of any one of claims 1 to 28, or a pharmaceutically acceptable salt thereof, wherein R⁶ is methyl, R⁷ is methyl, and n=1.

30. The compound of any one of claims 1 to 28, or a pharmaceutically acceptable salt thereof, wherein R⁶ is halo, R⁷ is halo, and n=1.

31. The compound of claim 30, or a pharmaceutically acceptable salt thereof, wherein R⁶ is fluoro, R⁷ is fluoro, and n=1.

32. The compound of any one of claims 1-25, 27 or 29-31, or a pharmaceutically acceptable salt thereof, wherein R⁴ and R⁶, together with the atom to which they are attached, form a bridged C₇-C₈ heterocyclic ring fused to the pyrazole.

33. The compound of claim 32, or a pharmaceutically acceptable salt thereof, wherein R⁴ and R⁶, together with the atom to which they are attached, form a bridged C₇-C₈ heterocyclic ring fused to the pyrazole, and n=1.

34. The compound of any one of claims 1-25 or 28-31, or a pharmaceutically acceptable salt thereof, wherein R⁴ and R⁵, together with the atom to which they are attached, form a bridged C₇-C₈ heterocyclic ring fused to the pyrazole.

35. The compound of claim 34, or a pharmaceutically acceptable salt thereof, wherein R⁴ and R⁵, together with the atom to which they are attached, form a bridged C₇₋₈ heterocyclic ring fused to the pyrazole, and n=1.

36. The compound of any one of claims 1 to 35, or a pharmaceutically acceptable salt thereof, wherein n is 1.

37. The compound of any one of claims 1 to 36, or a pharmaceutically acceptable salt thereof, wherein n is 2.

38. The compound of any one of claims 1 to 24, or a pharmaceutically acceptable salt thereof, wherein 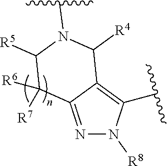
is

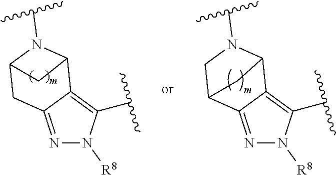

39. The compound of claim 38, or a pharmaceutically acceptable salt thereof, wherein 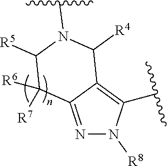
is 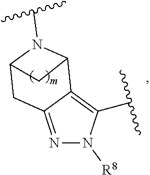
and m is 2.

40. The compound of any one of claims 1 to 24, or a pharmaceutically acceptable salt thereof, wherein 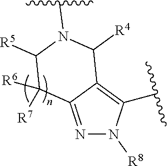
is

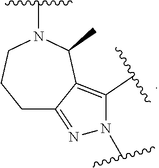 [SMILES: *c1c2c(nn1*)CCCN(*)[C@H]2C]

41. The compound of any one of claims 1 to 24, or a pharmaceutically acceptable salt thereof, wherein 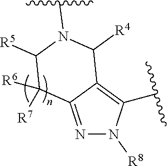
is

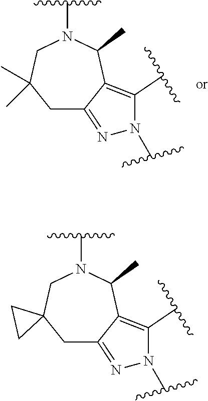 [SMILES: *c1c2c(nn1*)CC(C)(C)CN(*)[C@H]2C ; *c1c2c(nn1*)CC1(CC1)CN(*)[C@H]2C]

42. The compound of any one of claims 1 to 24, or a pharmaceutically acceptable salt thereof, wherein 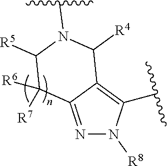
is 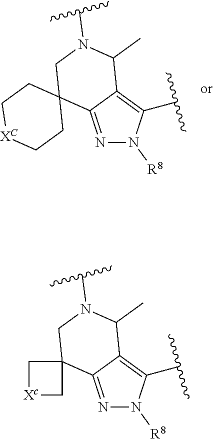
and X^{c} is O or NR⁹ wherein R⁹ is H, C₁-C₆ alkyl, SO₂—(C₁-C₆ alkyl), ((C₁-C₆ alkylenyl)O)ₜ(C₁-C₆ alkyl), —(C₁-C₆ alkylenyl)(C₃-C₈cycloalkyl), —(C)(O)(C₁-C₆ alkyl), —(CH₂)aryl, C₃-C₈ cycloalkyl, —(CH₂)(C₃-C₈ cycloalkyl), or C₁-C₆ alkyl, wherein one or more of the hydrogen atoms of the C₁-C₆ alkyl may be substituted with fluoro, and t is 1, 2, 3, 4 or 5.

43. The compound of claim 42, or a pharmaceutically acceptable salt thereof, wherein X^{c} is O, NH, N(C₁-C₆ alkyl), N(C)(O)(C₁-C₆ alkyl), N(SO₂—(C₁-C₆ alkyl)), or N((C₁-C₆ alkylenyl)O)ₜ(C₁-C₆ alkyl), wherein t is 1, 2, 3, 4 or 5.

44. The compound of claim 42 or 43, or a pharmaceutically acceptable salt thereof, wherein X^{c} is NR⁹ and R⁹ is —(C₁-C₆ alkylenyl)(C₃-C₈cycloalkyl), C₃-C₈ cycloalkyl, —C(O)C₁-C₆ alkyl, —((C₁-C₆ alkylenyl)O)ₜ(C₁-C₆ alkyl), or —S(O)₂(C₁-C₆ alkyl) wherein one or more of the hydrogen atoms of the C₁-C₆ alkyl may be substituted with fluoro or hydroxyl, and wherein t is 1, 2, 3, 4 or 5.

45. The compound of claim 42 or 43, or a pharmaceutically acceptable salt thereof, wherein X^{c} is NR⁹ and R⁹ is —(CH₂)aryl, —(CH₂)(C₃-C₈cycloalkyl), or C₁-C₆ alkyl wherein one or more of the hydrogen atoms of the C₁-C₆ alkyl may be substituted with fluoro.

46. The compound of claim 42 or 43, or a pharmaceutically acceptable salt thereof, wherein X^{c} is NR⁹ and R⁹ is H, —CH₂CF₃, —CH₂CHF₂, —CH₂C(CH₃)₂OH, cyclopentyl, phenylmethyl, —C(O)CH₃, —C(O)C(CH₃)₃, —C(O)CHF₂, —C(O)CF₂CHF₂, —(CH₂CH₂O)₄(CH₃), S(O)₂CH₃, or

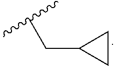 [SMILES: *CC1CC1]

47. The compound any one of claims 42 to 46, or a pharmaceutically acceptable salt thereof, wherein 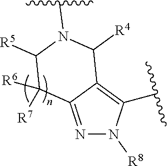
is

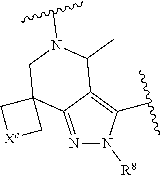

48. The compound of any one of claims 1 to 24, or a pharmaceutically acceptable salt thereof, wherein 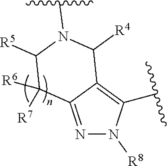
is 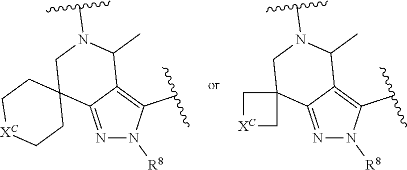
and X^{c} is O, NH, N(C₁-C₆ alkyl), N(C)(O)(C₁-C₆ alkyl), N(SO₂—(C₁-C₆ alkyl)).

49. The compound of any one of claims 1 to 48, or a pharmaceutically acceptable salt thereof, wherein 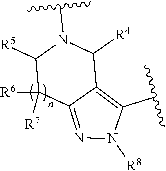
is

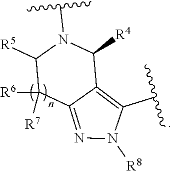

50. The compound of any one of claims 1 to 49, or a pharmaceutically acceptable salt thereof, wherein X is O.

51. The compound of any one of claims 1 to 49, or a pharmaceutically acceptable salt thereof, wherein X is N(CH₃) or

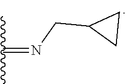 [SMILES: *=NCC1CC1]

52. The compound of any one of claims 1 to 51, or a pharmaceutically acceptable salt thereof, wherein R⁴, R⁵, R⁶ and R⁷ are each independently substituted with 0 to 2 substituents independently selected from halogen, —NH₂, aminoalkyl, diaminoalkyl, C₁-C₆ alkyl, and C₁-C₆ alkyl substituted with one or more halogen atoms, C₁-C₆ alkoxy, and hydroxy.

53. The compound of any one of claims 1 to 52, or a pharmaceutically acceptable salt thereof, wherein R^{y}, and R^{z}, together with the atom which they are attached, form a 5-7 membered heterocycloalkyl ring or a 5-6 membered heteroaryl.

54. The compound of any one of claims 1 to 52, or a pharmaceutically acceptable salt thereof, wherein R^{z} is H.

55. The compound of any one of claims 1 to 52, or a pharmaceutically acceptable salt thereof, wherein Y is 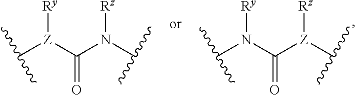
Z is CR^{x}, and R^{x} is H, halogen, or CH₃.

56. The compound of any one of claims 1 to 52, or a pharmaceutically acceptable salt thereof, wherein Y is 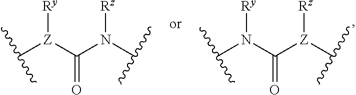
Z is CR^{x} and R^{x} is H and R^{y} is H.

57. The compound of any one of claims 1 to 52, or a pharmaceutically acceptable salt thereof, wherein Y is 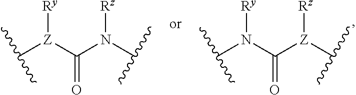
Z is CR^{x} and R^{x} is F and R^{y} is F.

58. The compound of and any one of claims 1 to 52, or a pharmaceutically acceptable salt thereof, wherein
Y is 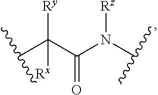
and
R^{x}, R^{y}, and R^{z} are each independently H, halogen, or CH₃.

59. The compound of any one of claims 52, 55 or 58, or a pharmaceutically acceptable salt thereof, wherein the halogen is fluoro.

60. The compound of any one of claims 1 to 52, or a pharmaceutically acceptable salt thereof, wherein Y is

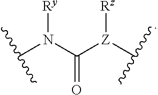

61. The compound of any one of claims 1 to 52, or a pharmaceutically acceptable salt thereof, wherein Y is 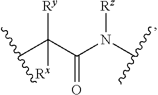
and
R^{x}, R^{y}, and R^{z} are each independently H, halogen, or CH₃; or R^{x} and R^{y}, together with the carbon to which they are attached, form a spiro C₃-C₆ cycloalkyl ring and R^{z} is H, halo, or C₁-C₆ alkyl.

62. The compound of any one of claims 1 to 52, or a pharmaceutically acceptable salt thereof, wherein Y is 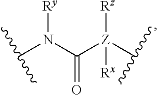
and
R^{x}, R^{y}, and R^{z} are each independently H, halogen, or CH₃.

63. The compound of any one of claims 1 to 52, or a pharmaceutically acceptable salt thereof, wherein
Y is 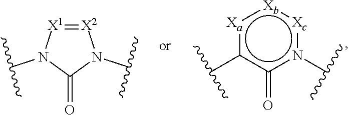
and
X¹ is N or C(H);
X² is N or C(H);
Xₐ is N or C(H);
X_{b} is N or C(H); and
X_{c} is N or C(H).

64. The compound of claim 63, or a pharmaceutically acceptable salt thereof, wherein at least one of X¹ or X² is N.

65. The compound of claim 63, or a pharmaceutically acceptable salt thereof, wherein at least one of Xₐ, X_{b}, and X_{c} is N.

66. The compound of any one of claims 1 to 52, or a pharmaceutically acceptable salt thereof, wherein Y is 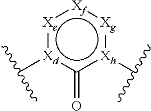
and 1-3 of X_{d}, Xₑ, X_{f}, X_{g}, and Xₕ, are nitrogen and the remaining are C or C(H).

67. The compound of claim 66, or a pharmaceutically acceptable salt thereof, wherein one or two of X_{d}, Xₑ, X_{f}, X_{g}, and Xₕ, are nitrogen and the remaining are C or C(H).

68. The compound of any one of claims 1 to 52, or a pharmaceutically acceptable salt thereof, wherein Y is:

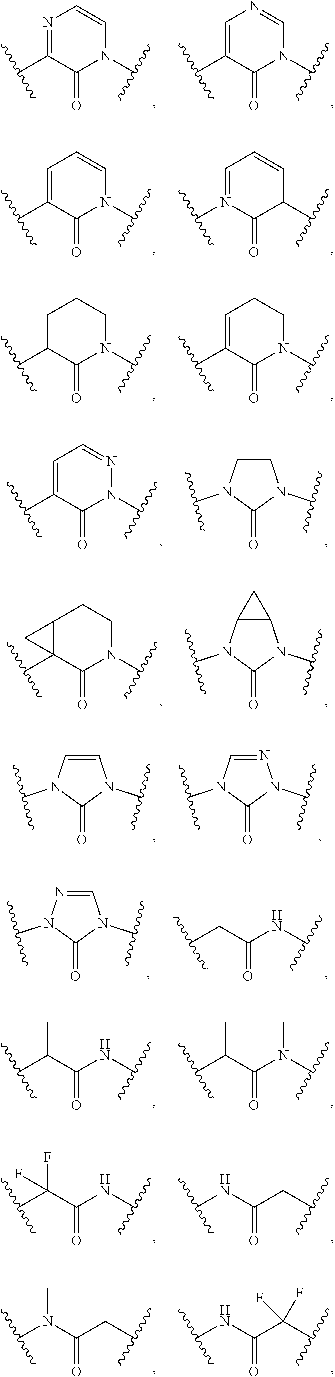

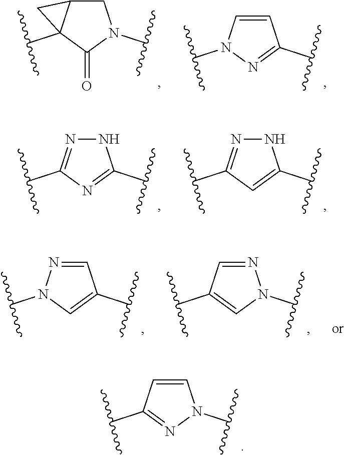

69. The compound of any one of claims 1 to 52, or a pharmaceutically acceptable salt thereof, wherein Y is

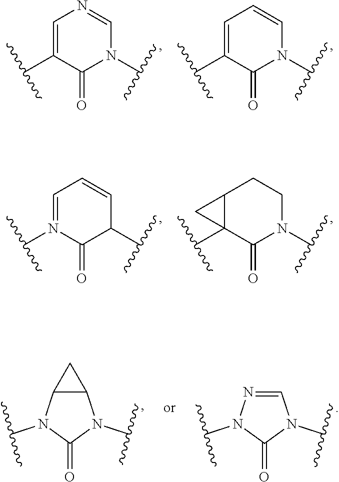

70. The compound of any one of claims 1 to 52, or a pharmaceutically acceptable salt thereof, wherein Y is:

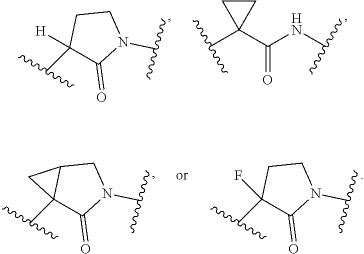 [SMILES: *C1CCN(*)C1=O ; *NC(=O)C1(*)CC1 ; *N1CC2CC2(*)C1=O ; *N1CCC(*)(F)C1=O]

71. The compound of any one of claims 1-20 or 22-52, or a pharmaceutically acceptable salt thereof, wherein Y is:

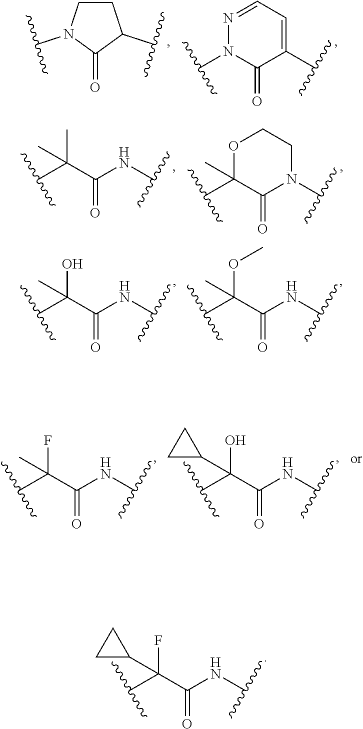

72. The compound of claim 68, or a pharmaceutically acceptable salt thereof, wherein Y is:

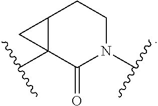 [SMILES: *N1CCC2CC2(*)C1=O]

73. The compound of claim 68, or a pharmaceutically acceptable salt thereof, wherein Y is:

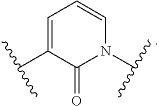 [SMILES: *c1cccn(*)c1=O]

74. The compound of claim 68, or a pharmaceutically acceptable salt thereof, wherein Y is:

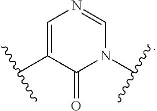 [SMILES: *c1cncn(*)c1=O]

75. The compound of claim 68, or a pharmaceutically acceptable salt thereof, wherein Y is:

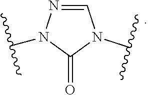 [SMILES: *n1cnn(*)c1=O]

76. The compound of claim 70, or a pharmaceutically acceptable salt thereof, wherein Y is:

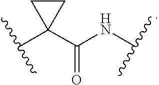 [SMILES: *NC(=O)C1(*)CC1]

77. The compound of claim 68, or a pharmaceutically acceptable salt thereof, wherein Y is:

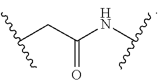 [SMILES: *CC(=O)N*]

78. The compound of claim 68, or a pharmaceutically acceptable salt thereof, wherein Y is

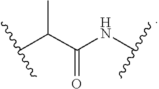 [SMILES: *NC(=O)C(*)C]

79. The compound of claim 71, or a pharmaceutically acceptable salt thereof, wherein Y is

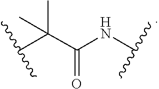 [SMILES: *NC(=O)C(*)(C)C]

80. The compound of claim 71, or a pharmaceutically acceptable salt thereof, wherein Y is

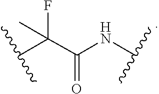 [SMILES: *NC(=O)C(*)(C)F]

81. The compound of claim 68, or a pharmaceutically acceptable salt thereof, wherein Y is 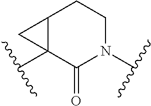 [SMILES: *N1CCC2CC2(*)C1=O]
and B is

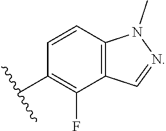 [SMILES: *c1ccc2c(cnn2C)c1F]

82. The compound of claim 68, or a pharmaceutically acceptable salt thereof, wherein Y is 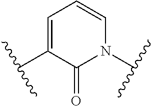 [SMILES: *c1cccn(*)c1=O]
and B is

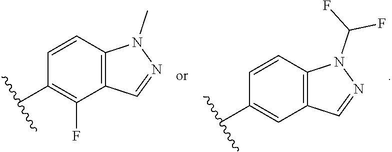 [SMILES: *c1ccc2c(cnn2C)c1F ; *c1ccc2c(cnn2C(F)F)c1]

83. The compound of claim 68, or a pharmaceutically acceptable salt thereof, wherein Y is 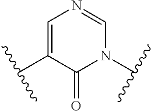 [SMILES: *c1cncn(*)c1=O]
and B is

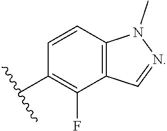 [SMILES: *c1ccc2c(cnn2C)c1F]

84. The compound of claim 68, or a pharmaceutically acceptable salt thereof, wherein Y is 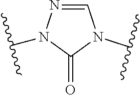 [SMILES: *n1cnn(*)c1=O]
and B is

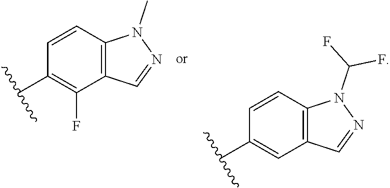 [SMILES: *c1ccc2c(cnn2C)c1F ; *c1ccc2c(cnn2C(F)F)c1]

85. The compound of any one of claims 72 to 80, or a pharmaceutically acceptable salt thereof, wherein
A is 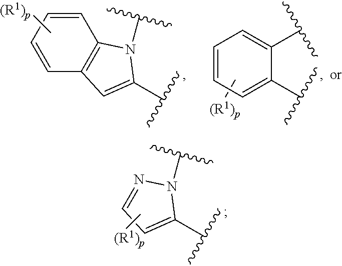
R¹ is 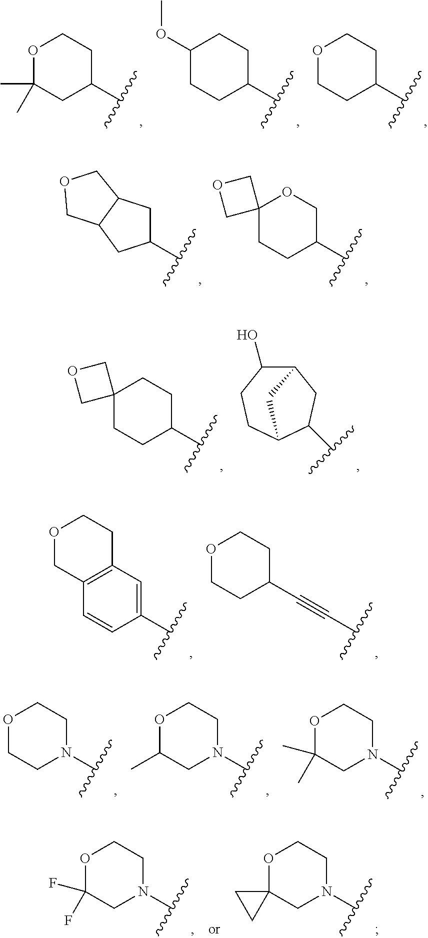
R² is H and R³ is H or methyl;
R⁴ is hydrogen, methyl, or ethyl;
R⁵ is hydrogen or methyl;
R⁴ and R⁵, together with the atom which they are attached, or R⁴ and R⁶, together with the atom which they are attached, form a bridged C₇₋₈ heterocyclic ring fused to the pyrazole; and
R⁶ is H, halo, or methyl, and R⁷ is H, halo, or methyl, or
R⁶ and R⁷ together with the atom to which they are attached, form a cyclopropyl ring or a cyclobutyl ring provided that if there are two R⁶ and R⁷ then only one set forms a ring and the other R⁶ and R⁷ is each independently H or methyl.

86. The compound of claim 85, or a pharmaceutically acceptable salt thereof, wherein B is 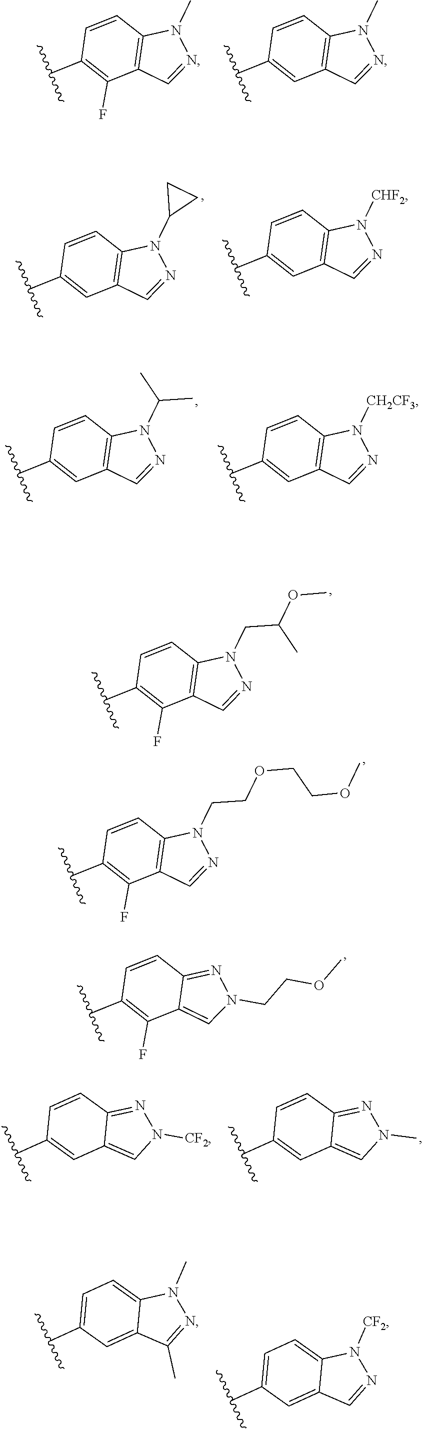 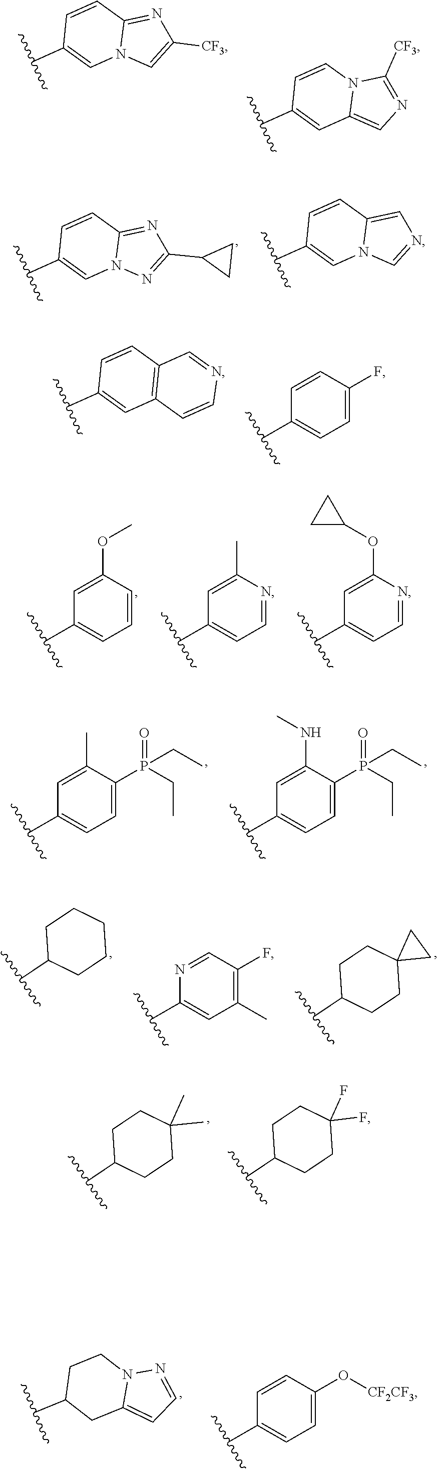 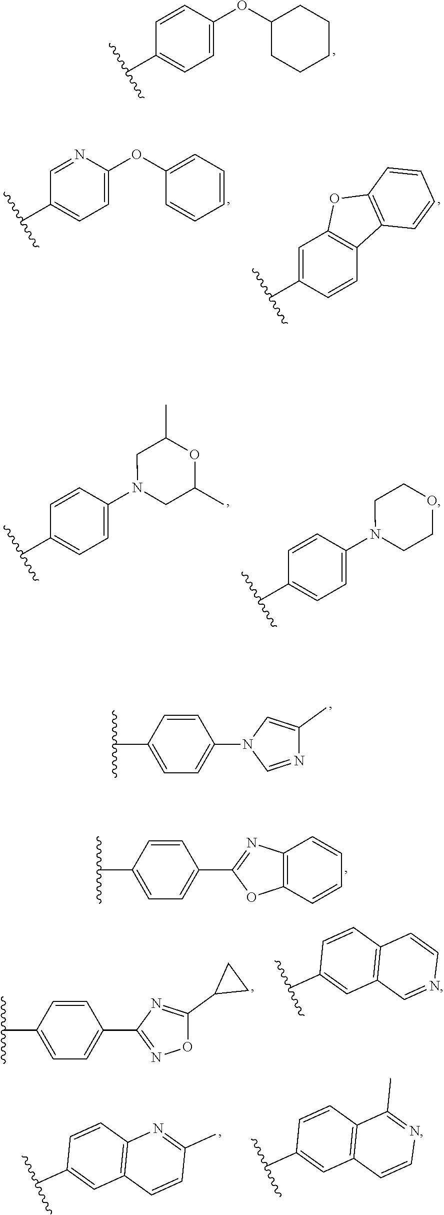 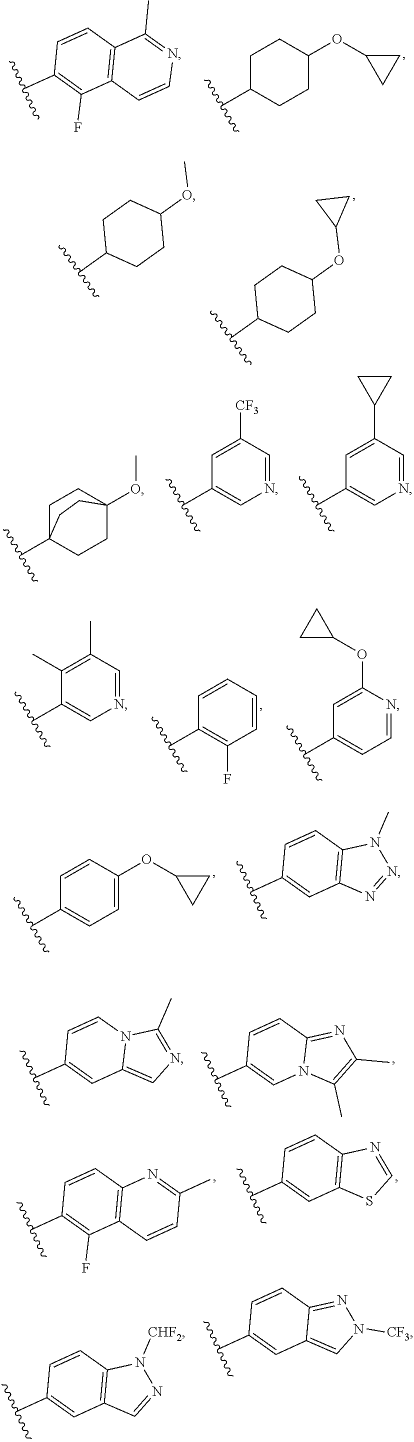 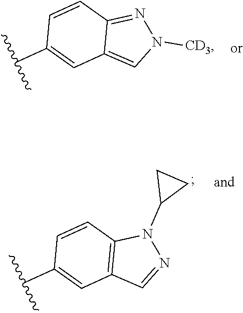 [SMILES: *c1ccc2nn(C([2H])([2H])[2H])cc2c1 ; *c1ccc2c(cnn2C2CC2)c1]
R⁸ is:

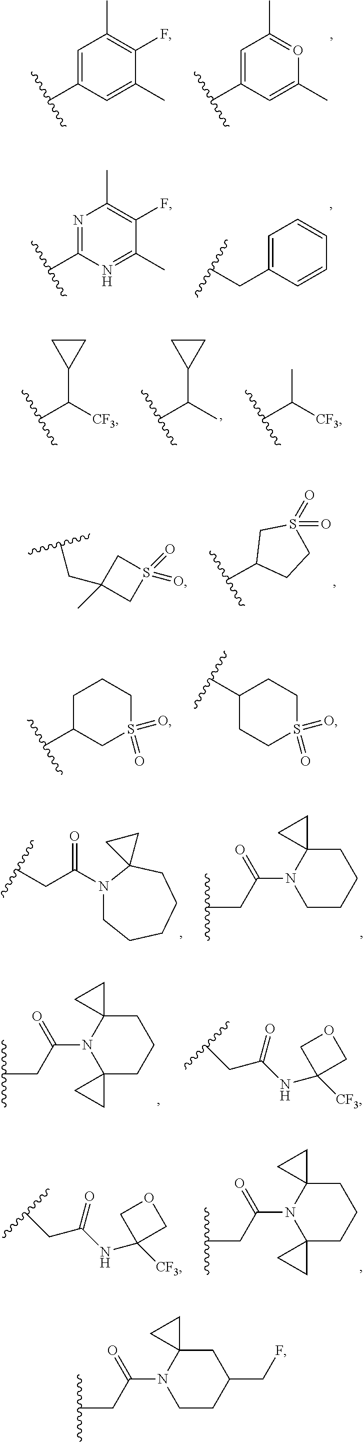

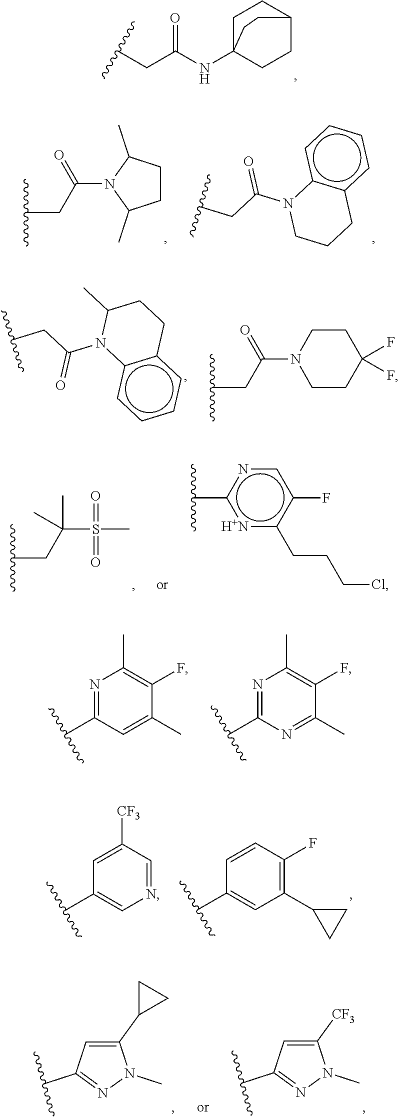

87. The compound of any one of claims 1 to 71, or a pharmaceutically acceptable salt thereof, wherein B is 5-13 membered heterocycloalkyl, aryl or heteroaryl, and B is substituted by 0, 1 or 2 substituents, each independently selected from C₁-C₃ alkyl, halo, —O(C₁-C₃ alkyl), —C₁-C₆ alkylenyl-O—(C₁-C₃ alkyl), C₁-C₅ alkylenyl-O—C₁-C₃ alkylenyl-O—C₁-C₃ alkyl, —NH(C₁-C₃ alkyl), —O(C₃-C₆ cycloalkyl), and —O(C₆-C₁₀ aryl), wherein one or more of the hydrogen atoms of the —C₁-C₃ alkyl or —O(C₁-C₃ alkyl) may be substituted with fluoro.

88. The compound of any one of claims 1 to 71, or a pharmaceutically acceptable salt thereof, wherein B is substituted by 1 or 2 substituents, each independently selected from fluoro, methyl, —CHF₂, cyclopropyl, —OCF₂CF₃,

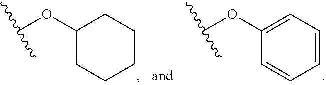 [SMILES: *OC1CCCCC1 ; *Oc1ccccc1]

89. The compound of any one of claims 1 to 71, or a pharmaceutically acceptable salt thereof, wherein B is 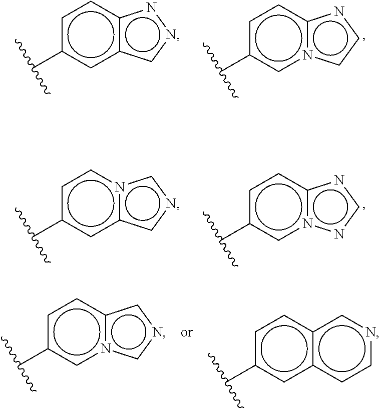
and B is substituted by 0, 1 or 2 substituents, as recited in claim 1.

90. The compound of claim 89, or a pharmaceutically acceptable salt thereof, wherein B is substituted by 1 or 2 instances of C₁-C₃ alkyl.

91. The compound of any one of claims 1 to 71, or a pharmaceutically acceptable salt thereof, wherein B is 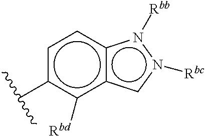
and
one of R^{bb} or R^{bc} is C₁-C₃ alkyl or heteroalkyl, and the other is not present,
R^{bd} is hydrogen or halogen,
wherein one or more of the hydrogen atoms of the C₁-C₃ alkyl or heteroalkyl may be substituted with fluoro.

92. The compound of claim 91, or a pharmaceutically acceptable salt thereof, wherein the heteroalkyl is —O(C₁-C₃ alkyl).

93. The compound of claim 87, or a pharmaceutically acceptable salt thereof, wherein B is 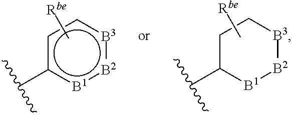
wherein at least two of B¹, B² and B³ are CH or CH₂, and the third is CH, CH₂, N or NH, and R^{be} represents one, or two optional ring substituents, which can be the same or different at each occurrence and wherein each occurrence of R^{be} is independently selected from C₁-C₃ alkyl, halo, —O(C₁-C₃ alkyl), —O(C₃-C₆ cycloalkyl), —O(C₆-C₁₀ aryl) and phosphorus atom substituted with oxo and/or C₁-C₃ alkyl, wherein one or more of the hydrogen atoms of the —C₁-C₃ alkyl or —O(C₁-C₃ alkyl) is optionally substituted with fluoro.

94. The compound of any one of claims 1-71 or 87-93, or a pharmaceutically acceptable salt thereof, wherein B is substituted with C₁-C₃ fluoroalkyl.

95. The compound of any one of claims 1-71 or 87-93, or a pharmaceutically acceptable salt thereof, wherein B is substituted by 1 or 2 substituents, each independently selected from fluoro, methyl, —CHF₂, CF₃, —CH₂CF₃, —OCF₂CF₃, —CH₂CH₂—O—CH₂CH₂—O—CH₃, —CH₂CH₂—O—CH₃, —OCH₃,

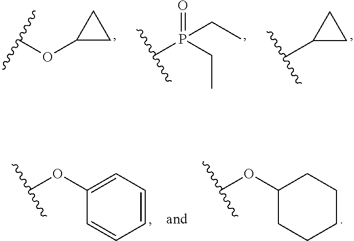

96. The compound of any one of claims 1-71 or 87-93, or a pharmaceutically acceptable salt thereof, wherein B is substituted by 1 or 2 substituents, each independently selected from fluoro, methyl, —CHF₂, CF₃, —CH₂CF₃, —OCF₂CF₃, —CH₂CH₂—O—CH₂CH₂—O—CH₃, —CH₂CH₂—O—CH₃, —OCH₃, —NH(CH₃),

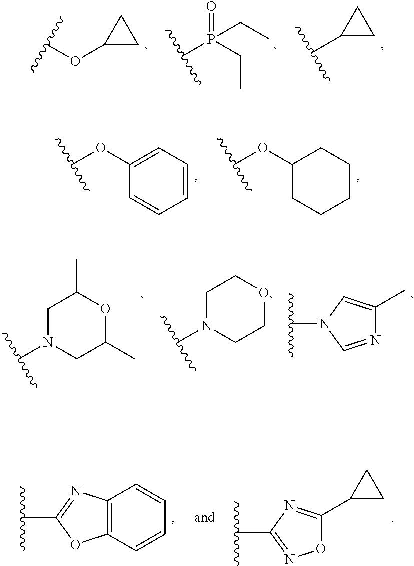

97. The compound of any one of claims 1 to 71, or a pharmaceutically acceptable salt thereof, wherein B is

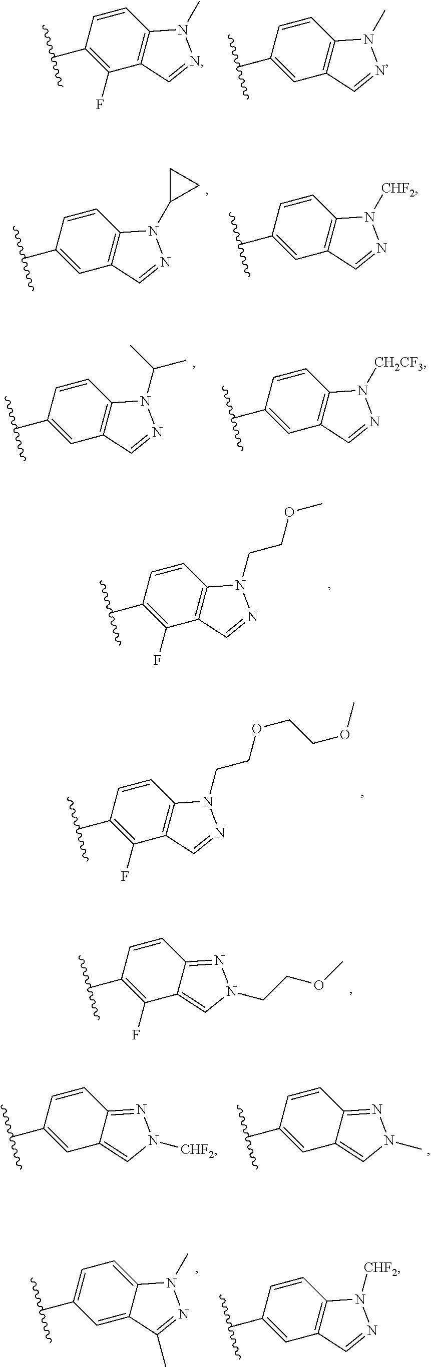

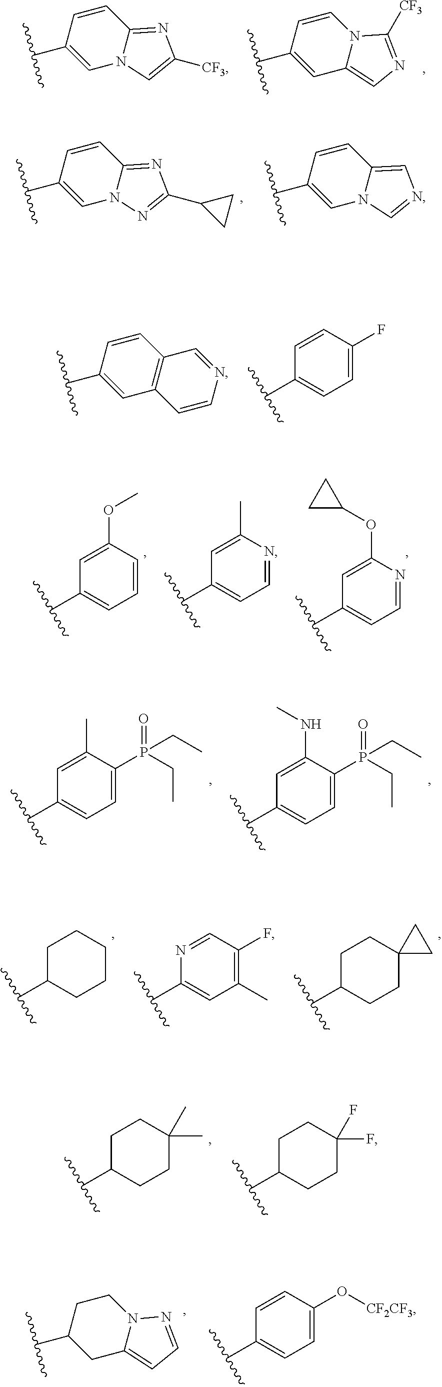

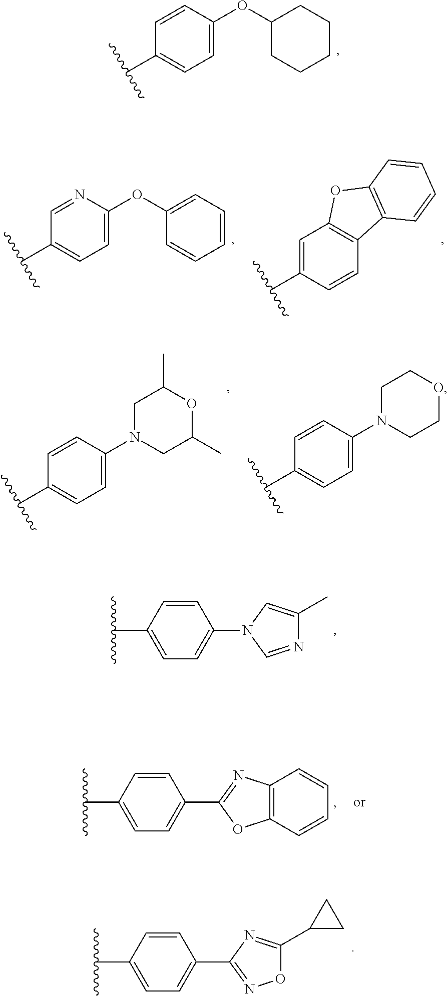

98. The compound of any one of claims 1 to 71, or a pharmaceutically acceptable salt thereof, wherein B is

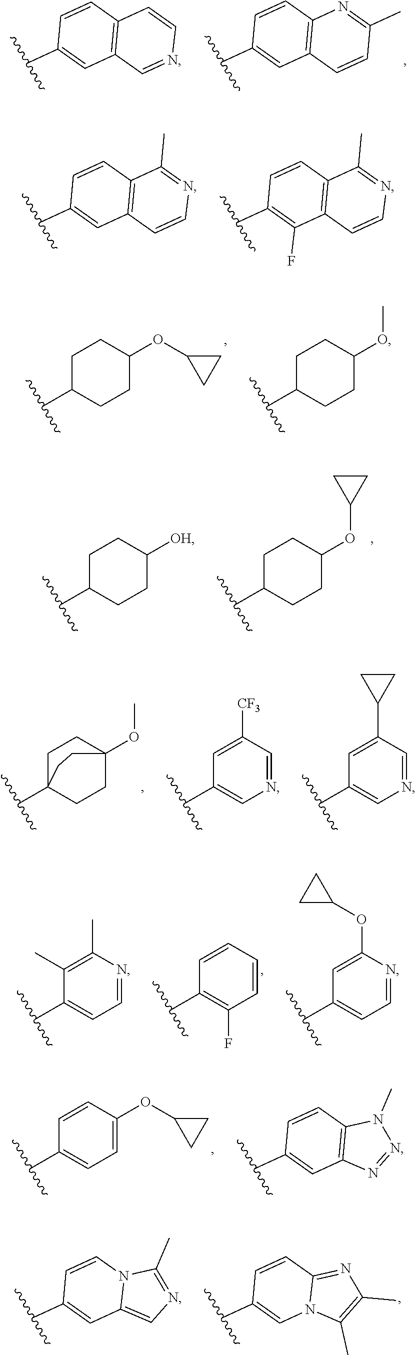

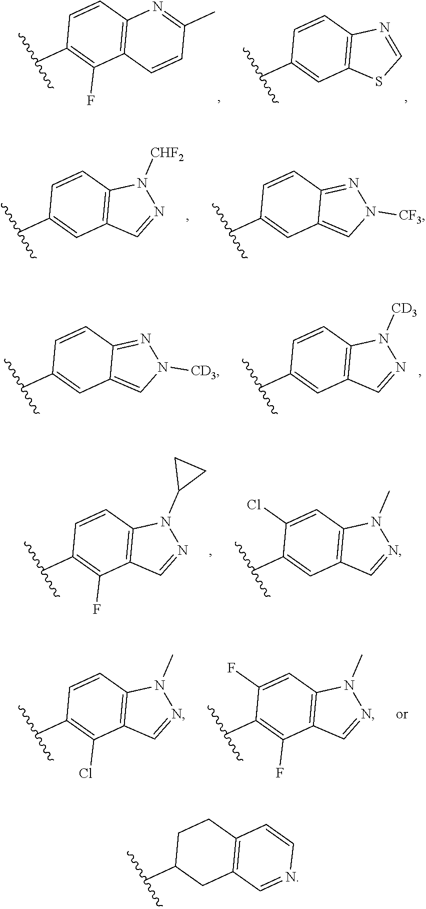

99. The compound of claim 98, or a pharmaceutically acceptable salt thereof, wherein B is

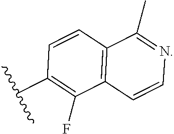 [SMILES: *c1ccc2c(C)nccc2c1F]

100. The compound of any one of claims 1-85 or 87-96, or a pharmaceutically acceptable salt thereof, wherein R⁸ is substituted by 1 or 2 substituents, each independently selected from fluoro, methyl, CF₃, cyclopropyl, and oxygen.

101. The compound of any one of claims 1 to 99, or a pharmaceutically acceptable salt thereof, wherein if R^{8a} is 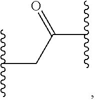 [SMILES: *CC(*)=O]
then R^{8b} is heteroaryl or 4-10 membered heterocycloalkyl with at least one nitrogen, and R^{8a} form a bond with the nitrogen of the heteroaryl or 4-10 membered heterocycloalkyl.

102. The compound of any one of claims 1 to 99, or a pharmaceutically acceptable salt thereof, wherein R⁸ is:

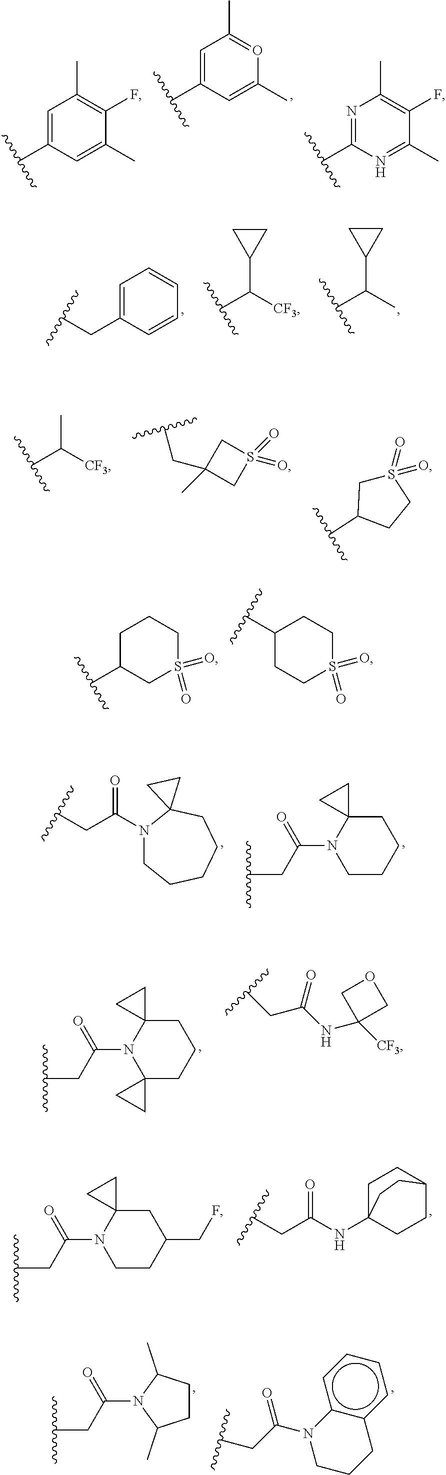

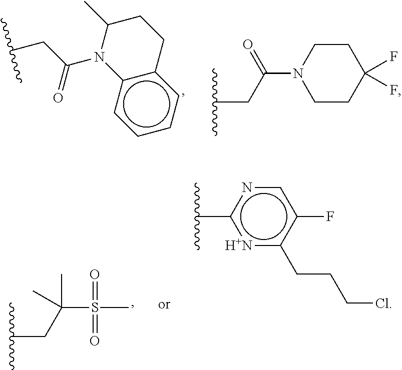

103. The compound of claim 100 or 101, or a pharmaceutically acceptable salt thereof, wherein R⁸ is:

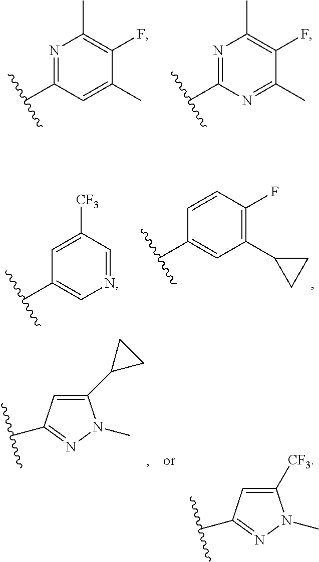

104. The compound of claim 102, or a pharmaceutically acceptable salt thereof wherein R⁸ is:

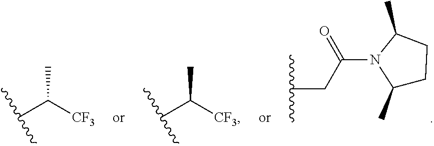

105. The compound of any one of claims 1-71 or 87-104, or a pharmaceutically acceptable salt thereof, wherein the compound of Formula I has the formula II, IIa, III, IV, IVa, IVb, V, Va, Vb, VI, VIa, VIb, VII, VIIa, or VIII and R^{z1} is H or C₁-C₆ alkyl and R^{z2} is H or C₁-C₆ alkyl,

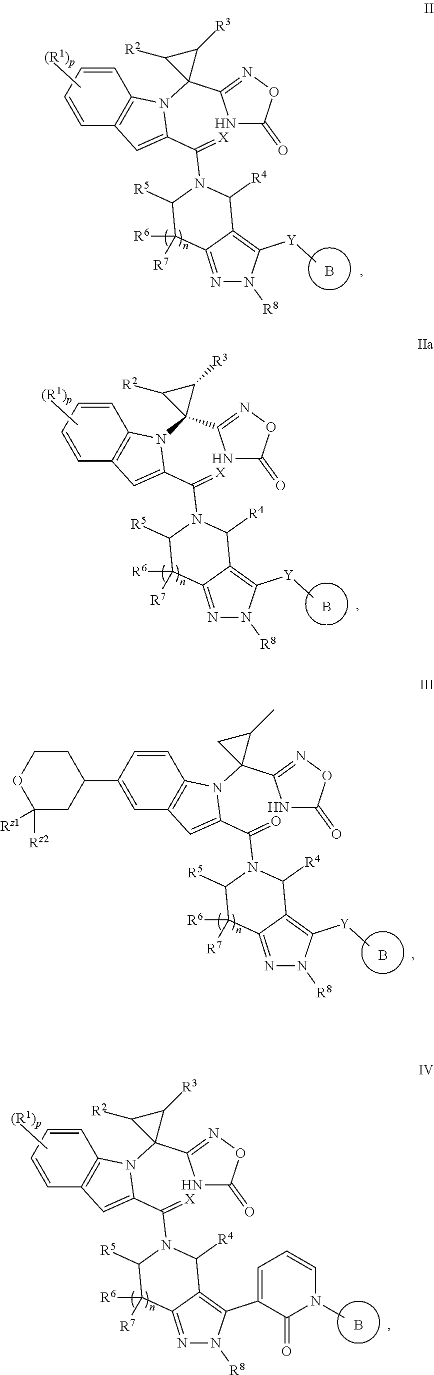

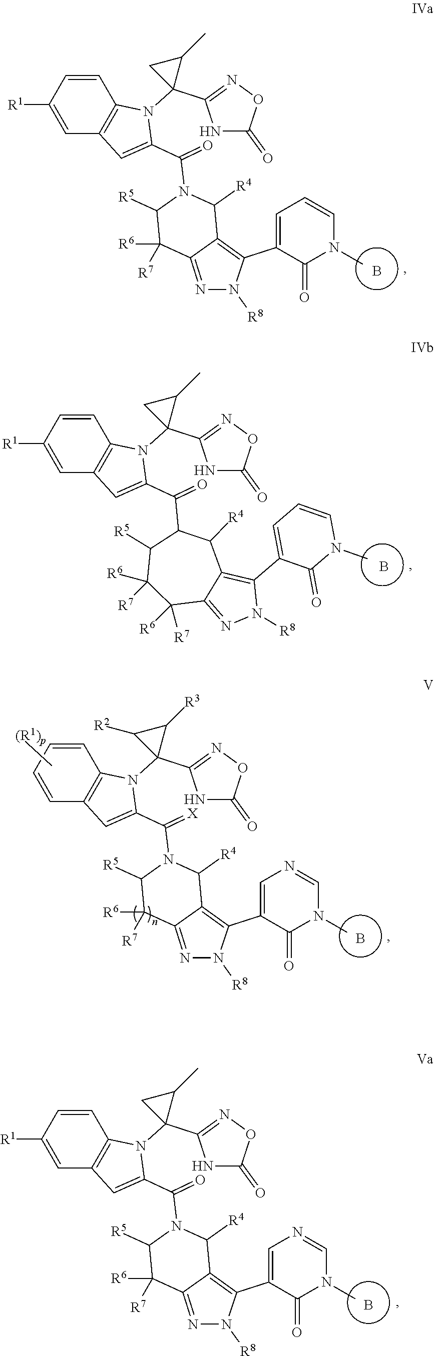

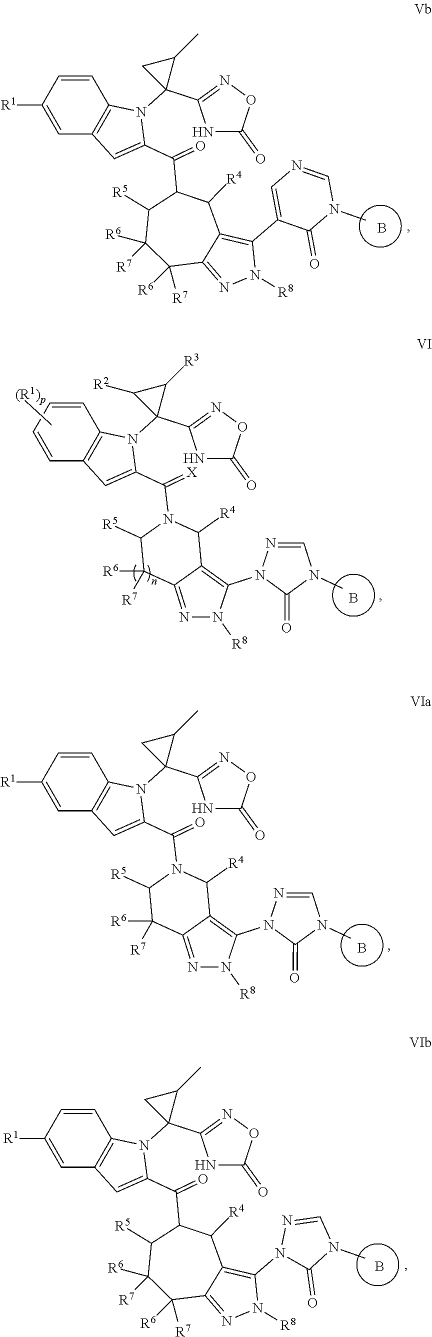

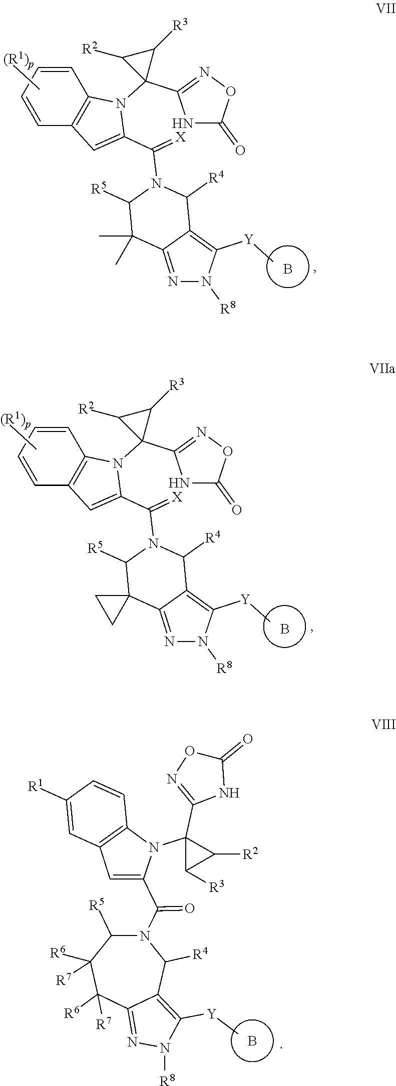

106. The compound of any one of claims 1-71, 87-98 or 100-104, or a pharmaceutically acceptable salt thereof, wherein the compound of Formula I has the formula IX, IXa or IXb:

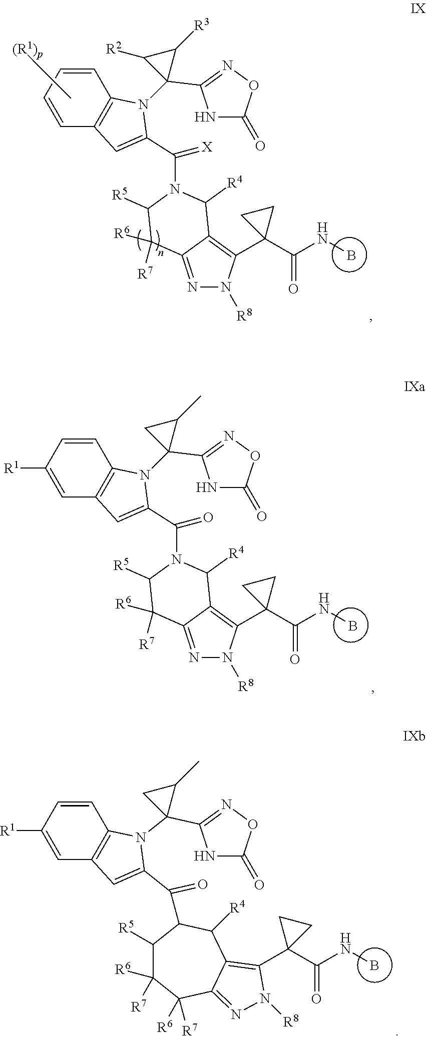

107. The compound of any one of claims 1-71 or 87-104, or a pharmaceutically acceptable salt thereof, wherein the compound of Formula I has the formula X, Xa or Xb:

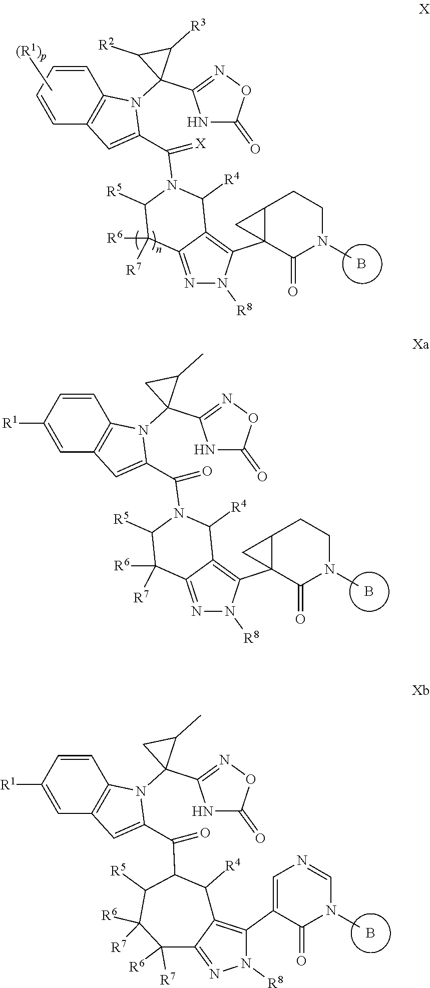

108. The compound of any one of claims 1-71, 87-98 or 100-106, or a pharmaceutically acceptable salt thereof, wherein
R⁸ is other than

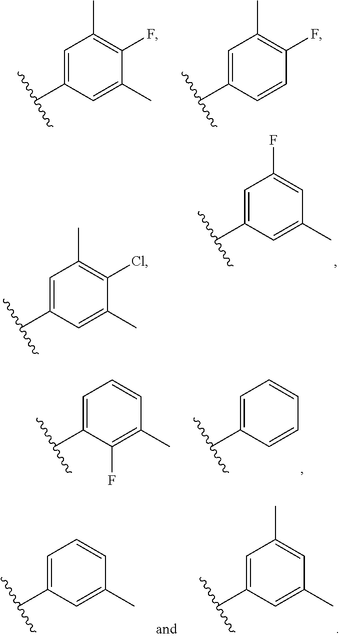

109. The compound of any one of claims 1-71, 87-98, 100-106 or 108, or a pharmaceutically acceptable salt thereof, wherein
R¹ is other than

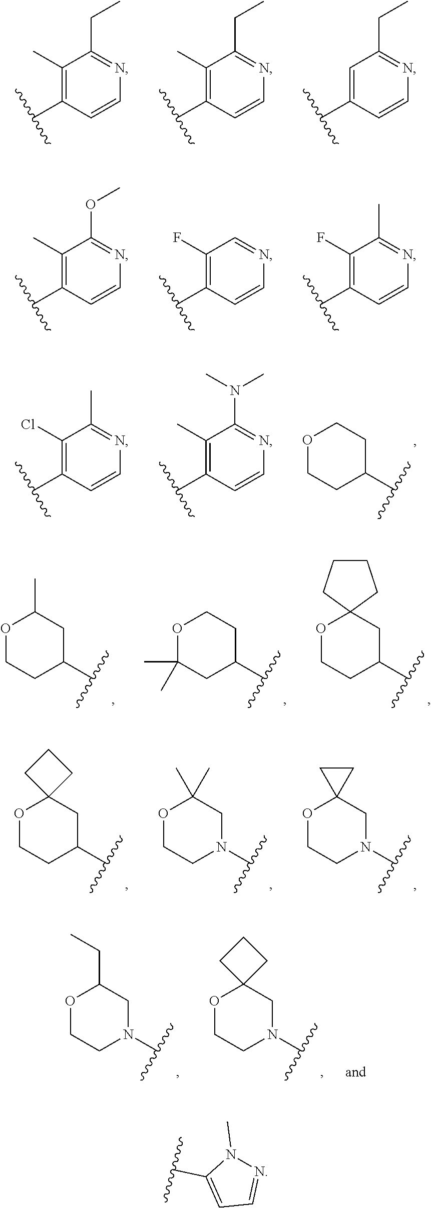

110. A compound selected from:
3-((1S,2S)-1-(2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4,7,7-trimethyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3′-(3-(4-fluoro-1-methyl-1H-indazol-5-yl)-2-oxo-2,3-dihydro-1H-imidazol-1-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-2,4,5,6,7,8-hexahydropyrazolo[4,3-c]azepine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-2,4,5,6,7,8-hexahydropyrazolo[4,3-c]azepine-5-carbonyl)-1H-indol-1-yl)-2-methylcyclopropyl)-1,2,4-oxadiazol-5(4H)-one;
ethyl 2-((S)-3-(3-(4-fluoro-1-methyl-1H-indazol-5-yl)-2-oxo-2,3-dihydro-1H-imidazol-1-yl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-2-yl)acetate;
(S)-3-(1-(5-bromo-2-(3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)cyclopropyl)-1,2,4-oxadiazol-5(4H)-one;
3-((1S,2S)-1-(2-((S)-2-benzyl-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-((S)-1-cyclopropylethyl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-((R)-1-cyclopropylethyl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-3-(3-(4-fluoro-1-methyl-1H-indazol-5-yl)-2-oxo-2,3-dihydro-1H-imidazol-1-yl)-4-methyl-2-(2-methyl-2-(methylsulfonyl)propyl)-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(5-(2,2-dimethyltetrahydro-2H-pyran-4-yl)-2-((S)-2-(1,1-dioxidotetrahydro-2H-thiopyran-4-yl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(2-((S)-3-(3-(4-fluoro-1-methyl-1H-indazol-5-yl)-2-oxo-2,3-dihydro-1H-imidazol-1-yl)-4-methyl-2-((3-methyl-1,1-dioxidothietan-3-yl)methyl)-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-3′-(3-(4-fluoro-1-methyl-1H-indazol-5-yl)-2-oxo-2,3-dihydro-1H-imidazol-1-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4,7,7-trimethyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
(4′S)-5′-(5-(2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-3′-(3-(4-fluoro-1-methyl-1H-indazol-5-yl)-2-oxo-2,3-dihydro-1H-imidazol-1-yl)-2′-(4-fluoro-3,5-dimethylphenyl)-4′-methyl-2′,4′,5′,6′-tetrahydrospiro[cyclobutane-1,7′-pyrazolo[4,3-c]pyridin]-1′-ium;
3-((1S,2S)-1-(2-((S)-3′-(3-(4-fluoro-1-methyl-1H-indazol-5-yl)-2-oxo-2,3-dihydro-1H-imidazol-1-yl)-2′-(4-fluoro-3,5-dimethylphenyl)-4′-methyl-2′,4′,5′,6′-tetrahydrospiro[cyclobutane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(5-(2,2-dimethyltetrahydro-2H-pyran-4-yl)-2-((S)-3′-(3-(4-fluoro-1-(2-methoxyethyl)-1H-indazol-5-yl)-2-oxo-2,3-dihydro-1H-imidazol-1-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-1H-indol-1-yl)-2-methylcyclopropyl)-1,2,4-oxadiazol-5(4H)-one;
3-((1S,2S)-1-(5-(2,2-dimethyltetrahydro-2H-pyran-4-yl)-2-((S)-3′-(3-(4-fluoro-2-(2-methoxyethyl)-2H-indazol-5-yl)-2-oxo-2,3-dihydro-1H-imidazol-1-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-1H-indol-1-yl)-2-methylcyclopropyl)-1,2,4-oxadiazol-5(4H)-one;
3-((1S,2S)-1-(5-(2,2-dimethyltetrahydro-2H-pyran-4-yl)-2-((S)-3′-(3-(4-fluoro-1-(2-(2-methoxyethoxy)ethyl)-1H-indazol-5-yl)-2-oxo-2,3-dihydro-1H-imidazol-1-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-1H-indol-1-yl)-2-methylcyclopropyl)-1,2,4-oxadiazol-5(4H)-one;
3-((1S,2S)-1-(2-((S)-3′-(3-(4-fluoro-1-methyl-1H-indazol-5-yl)-2-oxo-2,3-dihydro-1H-imidazol-1-yl)-2′-(4-fluoro-3,5-dimethylphenyl)-4′-methyl-2,2′,3,4′,5,5′,6,6′-octahydrospiro[pyran-4,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-((S)-1-cyclopropyl-2,2,2-trifluoroethyl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-((R)-1-cyclopropyl-2,2,2-trifluoroethyl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-((R)-1-cyclopropylethyl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-((S)-2,2-dimethyltetrahydro-2H-pyran-4-yl)-1H-indol-1-yl)-2-methylcyclopropyl)-1,2,4-oxadiazol-5(4H)-one;
3-((1S,2S)-1-(2-((S)-2-((S)-1-cyclopropylethyl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-((S)-2,2-dimethyltetrahydro-2H-pyran-4-yl)-1H-indol-1-yl)-2-methylcyclopropyl)-1,2,4-oxadiazol-5(4H)-one;
3-((1S,2S)-1-(2-((S)-2-((R)-1-cyclopropylethyl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-((S)-2,2-dimethyltetrahydro-2H-pyran-4-yl)-1H-indol-1-yl)-2-methylcyclopropyl)-1,2,4-oxadiazol-5(4H)-one;
3-((1S,2S)-1-(2-((S)-2-((S)-1-cyclopropylethyl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-((S)-2,2-dimethyltetrahydro-2H-pyran-4-yl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-2-((S)-1,1-dioxidotetrahydro-2H-thiopyran-3-yl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-2-((R)-1,1-dioxidotetrahydro-2H-thiopyran-3-yl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-2-((S)-1,1-dioxidotetrahydro-2H-thiopyran-3-yl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-2-((R)-1,1-dioxidotetrahydro-2H-thiopyran-3-yl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(2-((S)-2-((S)-1,1-dioxidotetrahydrothiophen-3-yl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-((R)-1,1-dioxidotetrahydrothiophen-3-yl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-((S)-1,1-dioxidotetrahydrothiophen-3-yl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-((R)-1,1-dioxidotetrahydrothiophen-3-yl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((4S,7R)-3-(3-(4-fluoro-1-methyl-1H-indazol-5-yl)-2-oxo-2,3-dihydro-1H-imidazol-1-yl)-2-(4-fluoro-3,5-dimethylphenyl)-2,4,5,6,7,8-hexahydro-4,7-epiminocyclohepta[c]pyrazole-9-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((4R,7S)-3-(3-(4-fluoro-1-methyl-1H-indazol-5-yl)-2-oxo-2,3-dihydro-1H-imidazol-1-yl)-2-(4-fluoro-3,5-dimethylphenyl)-2,4,5,6,7,8-hexahydro-4,7-epiminocyclohepta[c]pyrazole-9-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((4S,7R)-3-(3-(4-fluoro-1-methyl-1H-indazol-5-yl)-2-oxo-2,3-dihydro-1H-imidazol-1-yl)-2-(4-fluoro-3,5-dimethylphenyl)-2,4,5,6,7,8-hexahydro-4,7-epiminocyclohepta[c]pyrazole-9-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((4R,7S)-3-(3-(4-fluoro-1-methyl-1H-indazol-5-yl)-2-oxo-2,3-dihydro-1H-imidazol-1-yl)-2-(4-fluoro-3,5-dimethylphenyl)-2,4,5,6,7,8-hexahydro-4,7-epiminocyclohepta[c]pyrazole-9-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-((2R,4r,6S)-2,6-dimethyltetrahydro-2H-pyran-4-yl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-((2R,4s,6S)-2,6-dimethyltetrahydro-2H-pyran-4-yl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-((2R,4r,6S)-2,6-dimethyltetrahydro-2H-pyran-4-yl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-((2R,4s,6S)-2,6-dimethyltetrahydro-2H-pyran-4-yl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-(1-(4-fluoro-1-methyl-1H-indazol-5-yl)-2-oxo-1,2-dihydropyridin-3-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-3-(1-(4-fluoro-1-methyl-1H-indazol-5-yl)-2-oxo-1,2-dihydropyridin-3-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-2-(4-fluoro-3,5-dimethylphenyl)-4-methyl-3-(1-(1-methyl-1H-indazol-5-yl)-2-oxo-1,2-dihydropyridin-3-yl)-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-2-(4-fluoro-3,5-dimethylphenyl)-4-methyl-3-(1-(1-methyl-1H-indazol-5-yl)-6-oxo-1,6-dihydropyrimidin-5-yl)-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(2-((S)-3-(1-(1-(difluoromethyl)-1H-indazol-5-yl)-2-oxo-1,2-dihydropyridin-3-yl)-2-(4-fluoro-3,5-dimethylphenyl)-4,7,7-trimethyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-(1-(2-(difluoromethyl)-2H-indazol-5-yl)-2-oxo-1,2-dihydropyridin-3-yl)-2-(4-fluoro-3,5-dimethylphenyl)-4,7,7-trimethyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-(4-fluoro-3,5-dimethylphenyl)-4-methyl-3-(1-(1-methyl-1H-indazol-5-yl)-2-oxo-1,2-dihydropyridin-3-yl)-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-(4-fluoro-3,5-dimethylphenyl)-4,7,7-trimethyl-3-(1-(2-methyl-2H-indazol-5-yl)-2-oxo-1,2-dihydropyridin-3-yl)-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-(1-(1,3-dimethyl-1H-indazol-5-yl)-2-oxo-1,2-dihydropyridin-3-yl)-2-(4-fluoro-3,5-dimethylphenyl)-4,7,7-trimethyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-(4-fluoro-3,5-dimethylphenyl)-4,7,7-trimethyl-3-(1-(1-methyl-1H-indazol-5-yl)-2-oxo-1,2-dihydropyridin-3-yl)-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3′-(1-(1,3-dimethyl-1H-indazol-5-yl)-2-oxo-1,2-dihydropyridin-3-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2′-(4-fluoro-3,5-dimethylphenyl)-4′-methyl-3′-(1-(2-methyl-2H-indazol-5-yl)-2-oxo-1,2-dihydropyridin-3-yl)-2′,4′,5′,6′-tetrahydrospiro[cyclopropane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-(1-(4-fluoro-1-methyl-1H-indazol-5-yl)-2-oxo-1,2-dihydropyridin-3-yl)-2-(4-fluoro-3,5-dimethylphenyl)-4,7,7-trimethyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-3-(1-(4-fluoro-1-methyl-1H-indazol-5-yl)-2-oxo-1,2-dihydropyridin-3-yl)-2-(4-fluoro-3,5-dimethylphenyl)-4,7,7-trimethyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(2-((S)-3-(1-(2-(difluoromethyl)-2H-indazol-5-yl)-2-oxo-1,2-dihydropyridin-3-yl)-2-(4-fluoro-3,5-dimethylphenyl)-4,7,7-trimethyl-4,5,6,7-tetrahydro-2H-pyrazolo[4,3-c]pyridine-5-carbonyl)-5-((S)-2,2-dimethyltetrahydro-2H-pyran-4-yl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-2-(4-fluoro-3,5-dimethylphenyl)-4,7,7-trimethyl-3-(1-(1-methyl-1H-indazol-5-yl)-2-oxo-1,2-dihydropyridin-3-yl)-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(2-((S)-2′-(4-fluoro-3,5-dimethylphenyl)-4′-methyl-3′-(1-(1-methyl-1H-indazol-5-yl)-2-oxo-1,2-dihydropyridin-3-yl)-2′,4′,5′,6′-tetrahydrospiro[cyclopropane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-(4-fluoro-3,5-dimethylphenyl)-4-methyl-3-(2-oxo-1-(2-(trifluoromethyl)imidazo[1,2-a]pyridin-6-yl)-1,2-dihydropyridin-3-yl)-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-(1-(2-cyclopropyl-[1,2,4]triazolo[1,5-a]pyridin-6-yl)-2-oxo-1,2-dihydropyridin-3-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-(4-fluoro-3,5-dimethylphenyl)-4-methyl-3-(2-oxo-1-(3-(trifluoromethyl)imidazo[1,5-a]pyridin-7-yl)-1,2-dihydropyridin-3-yl)-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-(1-(1-(difluoromethyl)-1H-indazol-5-yl)-2-oxo-1,2-dihydropyridin-3-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-(1-(1-(difluoromethyl)-1H-indazol-5-yl)-2-oxo-1,2-dihydropyridin-3-yl)-2-(4-fluoro-3,5-dimethylphenyl)-4-methyl-4,5,6,7-tetrahydro-2H-pyrazolo[4,3-c]pyridine-5-carbonyl)-5-((S)-2,2-dimethyltetrahydro-2H-pyran-4-yl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-2-(4-fluoro-3,5-dimethylphenyl)-4-methyl-3-(2′-methyl-2-oxo-2H-[1,4′-bipyridin]-3-yl)-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-2-(4-fluoro-3,5-dimethylphenyl)-3-(1-(imidazo[1,5-a]pyridin-6-yl)-2-oxo-1,2-dihydropyridin-3-yl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(2-((S)-3′-(2′-cyclopropoxy-2-oxo-2H-[1,4′-bipyridin]-3-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-(2′-cyclopropoxy-2-oxo-2H-[1,4′-bipyridin]-3-yl)-2-(4-fluoro-3,5-dimethylphenyl)-4,7,7-trimethyl-4,5,6,7-tetrahydro-2H-pyrazolo[4,3-c]pyridine-5-carbonyl)-5-((S)-2,2-dimethyltetrahydro-2H-pyran-4-yl)-1H-indol-1-yl)-2-methylcyclopropyl)-1,2,4-oxadiazol-5(4H)-one;
3-((1S,2S)-1-(2-((S)-3-(2′-cyclopropoxy-2-oxo-2H-[1,4′-bipyridin]-3-yl)-2-(4-fluoro-3,5-dimethylphenyl)-4,7,7-trimethyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3′-(2′-cyclopropoxy-2-oxo-2H-[1,4′-bipyridin]-3-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-5-((S)-2,2-dimethyltetrahydro-2H-pyran-4-yl)-1H-indol-1-yl)-2-methylcyclopropyl)-1,2,4-oxadiazol-5(4H)-one;
3-((1S,2S)-1-(2-((S)-3-(2′-cyclopropoxy-2-oxo-2H-[1,4′-bipyridin]-3-yl)-2-(4-fluoro-3,5-dimethylphenyl)-4-methyl-4,5,6,7-tetrahydro-2H-pyrazolo[4,3-c]pyridine-5-carbonyl)-5-((S)-2,2-dimethyltetrahydro-2H-pyran-4-yl)-1H-indol-1-yl)-2-methylcyclopropyl)-1,2,4-oxadiazol-5(4H)-one;
3-((1S,2S)-1-(2-((S)-3-(2′-cyclopropoxy-2-oxo-2H-[1,4′-bipyridin]-3-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one
3-((1S,2S)-1-(2-((S)-2-(4-fluoro-3,5-dimethylphenyl)-3-(1-(isoquinolin-6-yl)-2-oxo-1,2-dihydropyridin-3-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-(1-(4-(diethylphosphoryl)-3-(methylamino)phenyl)-2-oxo-1,2-dihydropyridin-3-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-(4-fluoro-3,5-dimethylphenyl)-4-methyl-3-(1-(4-morpholinophenyl)-2-oxo-1,2-dihydropyridin-3-yl)-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-7,7-difluoro-2-(4-fluoro-3,5-dimethylphenyl)-4-methyl-3-(1-(1-methyl-1H-indazol-5-yl)-2-oxo-1,2-dihydropyridin-3-yl)-4,5,6,7-tetrahydro-2H-pyrazolo[4,3-c]pyridine-5-carbonyl)-5-((S)-2,2-dimethyltetrahydro-2H-pyran-4-yl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-2-(4-fluoro-3,5-dimethylphenyl)-4,7,7-trimethyl-3-(1-(1-methyl-1H-indazol-5-yl)-6-oxo-1,6-dihydropyrimidin-5-yl)-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-2-(4-fluoro-3,5-dimethylphenyl)-4-methyl-3-(1-(1-methyl-1H-indazol-5-yl)-2-oxo-1,2-dihydropyridin-3-yl)-2,4,5,6,7,8-hexahydropyrazolo[4,3-c]azepine-5-carbonyl)-1H-indol-1-yl)-2-methylcyclopropyl)-1,2,4-oxadiazol-5(4H)-one;
3-((1S,2S)-1-(2-((S)-2-(4-fluoro-3,5-dimethylphenyl)-3-(1-(3-methoxyphenyl)-2-oxo-1,2-dihydropyridin-3-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-(4-fluoro-3,5-dimethylphenyl)-4-methyl-3-(2-oxo-1-(1-(2,2,2-trifluoroethyl)-1H-indazol-5-yl)-1,2-dihydropyridin-3-yl)-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-(1-(1-cyclopropyl-1H-indazol-5-yl)-2-oxo-1,2-dihydropyridin-3-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-(2-(4-fluoro-1-methyl-1H-indazol-5-yl)-3-oxo-2,3-dihydropyridazin-4-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-(4-fluoro-3,5-dimethylphenyl)-4-methyl-3-(2-(1-methyl-1H-indazol-5-yl)-3-oxo-2,3-dihydropyridazin-4-yl)-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-(4-fluoro-3,5-dimethylphenyl)-4,7,7-trimethyl-3-(2-(1-methyl-1H-indazol-5-yl)-3-oxo-2,3-dihydropyridazin-4-yl)-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2′-(4-fluoro-3,5-dimethylphenyl)-4′-methyl-3′-(2-(1-methyl-1H-indazol-5-yl)-3-oxo-2,3-dihydropyridazin-4-yl)-2′,4′,5′,6′-tetrahydrospiro[cyclopropane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-(4-fluoro-3,5-dimethylphenyl)-4-methyl-3-(1-(1-methyl-1H-indazol-5-yl)-6-oxo-1,6-dihydropyrimidin-5-yl)-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3′-(1-(4-fluoro-1-methyl-1H-indazol-5-yl)-2-oxo-1,2-dihydropyridin-3-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-3-(1-(4-fluoro-1-methyl-1H-indazol-5-yl)-6-oxo-1,6-dihydropyrimidin-5-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(2-((S)-3-(4-(4-fluoro-1-methyl-1H-indazol-5-yl)-5-oxo-4,5-dihydro-1H-1,2,4-triazol-1-yl)-2-(4-fluoro-3,5-dimethylphenyl)-4,7,7-trimethyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-(4-(4-fluoro-1-methyl-1H-indazol-5-yl)-5-oxo-4,5-dihydro-1H-1,2,4-triazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
(S)-3-(1-(2-(3-(4-(4-fluoro-1-methyl-1H-indazol-5-yl)-5-oxo-4,5-dihydro-1H-1,2,4-triazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)cyclopropyl)-1,2,4-oxadiazol-5(4H)-one;
3-((1S,2S)-1-(2-((S)-3′-(4-(4-fluoro-1-methyl-1H-indazol-5-yl)-5-oxo-4,5-dihydro-1H-1,2,4-triazol-1-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-3-(4-(4-fluoro-1-methyl-1H-indazol-5-yl)-5-oxo-4,5-dihydro-1H-1,2,4-triazol-1-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-3′-(4-(4-fluoro-1-methyl-1H-indazol-5-yl)-5-oxo-4,5-dihydro-1H-1,2,4-triazol-1-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-1H-indol-1-yl)-2-methylcyclopropyl)-1,2,4-oxadiazol-5(4H)-one;
N-(4-fluoro-1-methyl-1H-indazol-5-yl)-2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)acetamide;
2-((4′S)-5′-(5-(2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2′-(4-fluoro-3,5-dimethylphenyl)-4′-methyl-2′,4′,5′,6′-tetrahydrospiro[cyclobutane-1,7′-pyrazolo[4,3-c]pyridin]-3′-yl)-N-(4-fluoro-1-methyl-1H-indazol-5-yl)acetamide;
N-(4-fluoro-1-methyl-1H-indazol-5-yl)-2-((S)-2′-(4-fluoro-3,5-dimethylphenyl)-4′-methyl-5′-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-2′,4′,5′,6′-tetrahydrospiro[cyclobutane-1,7′-pyrazolo[4,3-c]pyridin]-3′-yl)acetamide;
2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N-(isochroman-7-yl)acetamide;
2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N-(4-(3-methyl-2-oxoimidazolidin-1-yl)phenyl)acetamide;
2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N-(1-methyl-1H-benzo[d]imidazol-6-yl)acetamide;
2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N-(1-methyl-1H-benzo[d][1,2,3]triazol-5-yl)acetamide;
2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N-(1-isopropyl-1H-indazol-5-yl)acetamide;
2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N-(4-morpholinophenyl)acetamide;
2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N-(2-methyl-2H-indazol-5-yl)acetamide;
2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N-(2-methylpyridin-4-yl)acetamide;
2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N-((1r,4S)-4-hydroxycyclohexyl)acetamide;
N-((1r,4S)-4-cyclopropoxycyclohexyl)-2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)acetamide;
N-(4,4-difluorocyclohexyl)-2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)acetamide
2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N-(4-(2-methoxyethoxy)cyclohexyl)acetamide;
2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N-(trans-4-methoxycyclohexyl)acetamide;
2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N-(1-methyl-1H-indazol-5-yl)acetamide;
2-((4S)-5-(5-(2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)-N-(4-fluoro-1-methyl-1H-indazol-5-yl)acetamide;
N-cyclohexyl-2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)acetamide;
N-(dibenzo[bd,]furan-2-yl)-2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)acetamide N-(4-(diethylphosphoryl)-3-(methylamino)phenyl)-2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)acetamide;
2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N—((R)-4,5,6,7-tetrahydropyrazolo[1,5-a]pyridin-5-yl)acetamide or 2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N—((S)-4,5,6,7-tetrahydropyrazolo[1,5-a]pyridin-5-yl)acetamide;
2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N—((R)-4,5,6,7-tetrahydropyrazolo[1,5-a]pyridin-5-yl)acetamide;
2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N—((S)-4,5,6,7-tetrahydropyrazolo[1,5-a]pyridin-5-yl)acetamide;
2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N-(4-(4-methyl-1H-imidazol-1-yl)phenyl)acetamide;
3-((1S,2S)-1-(2-((S)-3-(5-(4-fluoro-1-methyl-1H-indazol-5-yl)-1H-1,2,4-triazol-3-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-3-(5-(4-fluoro-1-methyl-1H-indazol-5-yl)-1H-1,2,4-triazol-3-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
(S)-3-(1-(2-(3-(5-(4-fluoro-1-methyl-1H-indazol-5-yl)-1H-1,2,4-triazol-3-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)cyclopropyl)-1,2,4-oxadiazol-5(4H)-one;
3-((1S,2S)-1-(2-((S)-3′-(3-(4-fluoro-1-methyl-1H-indazol-5-yl)-1H-pyrazol-1-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-3′-(3-(4-fluoro-1-methyl-1H-indazol-5-yl)-1H-pyrazol-1-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-1H-indol-1-yl)-2-methylcyclopropyl)-1,2,4-oxadiazol-5(4H)-one;
3-((1S,2S)-1-(2-((S)-3-(3-(4-fluoro-1-methyl-1H-indazol-5-yl)-1H-pyrazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
(S)-3-(1-(2-(3-(3-(4-fluoro-1-methyl-1H-indazol-5-yl)-1H-pyrazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)cyclopropyl)-1,2,4-oxadiazol-5(4H)-one;
2,2-difluoro-2-(4-fluoro-1-methyl-1H-indazol-5-yl)-N—((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)acetamide:
2,2-difluoro-2-(4-fluoro-1-methyl-1H-indazol-3-yl)-N—((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)acetamide;
N—((S)-5-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2-(4-fluoro-3,5-dimethylphenyl)-4-methyl-4,5,6,7-tetrahydro-2H-pyrazolo[4,3-c]pyridin-3-yl)-2,2-difluoro-2-(4-fluoro-1-methyl-1H-indazol-5-yl)acetamide;
(S)-2-(4-fluoro-1-methyl-1H-indazol-5-yl)-N-(2-(4-fluoro-3,5-dimethylphenyl)-4-methyl-5-(1-(1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)acetamide;
2-(4-fluoro-1-methyl-1H-indazol-5-yl)-N—((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N-methylacetamide;
3-((1S,2S)-1-(2-((S)-3-((1S,5R)-4-(4-fluoro-1-methyl-1H-indazol-5-yl)-3-oxo-2,4-diazabicyclo[3.1.0]hexan-2-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-((1R,5S)-4-(4-fluoro-1-methyl-1H-indazol-5-yl)-3-oxo-2,4-diazabicyclo[3.1.0]hexan-2-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-((1S,5R)-4-(4-fluoro-1-methyl-1H-indazol-5-yl)-3-oxo-2,4-diazabicyclo[3.1.0]hexan-2-yl)-2-(4-fluoro-3,5-dimethylphenyl)-4,7,7-trimethyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-((1R,5S)-4-(4-fluoro-1-methyl-1H-indazol-5-yl)-3-oxo-2,4-diazabicyclo[3.1.0]hexan-2-yl)-2-(4-fluoro-3,5-dimethylphenyl)-4,7,7-trimethyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3′-((1S,5R)-4-(4-fluoro-1-methyl-1H-indazol-5-yl)-3-oxo-2,4-diazabicyclo[3.1.0]hexan-2-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3′-((1R,5S)-4-(4-fluoro-1-methyl-1H-indazol-5-yl)-3-oxo-2,4-diazabicyclo[3.1.0]hexan-2-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-(4-fluoro-3,5-dimethylphenyl)-4-methyl-3-((1S,5R)-4-(1-methyl-1H-indazol-5-yl)-3-oxo-2,4-diazabicyclo[3.1.0]hexan-2-yl)-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-(4-fluoro-3,5-dimethylphenyl)-4-methyl-3-((1R,5S)-4-(1-methyl-1H-indazol-5-yl)-3-oxo-2,4-diazabicyclo[3.1.0]hexan-2-yl)-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-2-(4-fluoro-3,5-dimethylphenyl)-4-methyl-3-((1S,5R)-4-(1-methyl-1H-indazol-5-yl)-3-oxo-2,4-diazabicyclo[3.1.0]hexan-2-yl)-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-2-(4-fluoro-3,5-dimethylphenyl)-4-methyl-3-((1R,5S)-4-(1-methyl-1H-indazol-5-yl)-3-oxo-2,4-diazabicyclo[3.1.0]hexan-2-yl)-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-3-((1S,5R)-4-(4-fluoro-1-methyl-1H-indazol-5-yl)-3-oxo-2,4-diazabicyclo[3.1.0]hexan-2-yl)-2-(4-fluoro-3,5-dimethylphenyl)-4,7,7-trimethyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-3-((1R,5S)-4-(4-fluoro-1-methyl-1H-indazol-5-yl)-3-oxo-2,4-diazabicyclo[3.1.0]hexan-2-yl)-2-(4-fluoro-3,5-dimethylphenyl)-4,7,7-trimethyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-3′-((1S,5R)-4-(4-fluoro-1-methyl-1H-indazol-5-yl)-3-oxo-2,4-diazabicyclo[3.1.0]hexan-2-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-3′-((1R,5S)-4-(4-fluoro-1-methyl-1H-indazol-5-yl)-3-oxo-2,4-diazabicyclo[3.1.0]hexan-2-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-1H-indol-1-yl)-2-methylcyclopropyl)-1,2,4-oxadiazol-5(4H)-one;
3-((1S,2S)-1-(2-((S)-3′-((1S,5R)-4-(4-(diethylphosphoryl)-3-(methylamino)phenyl)-3-oxo-2,4-diazabicyclo[3.1.0]hexan-2-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3′-((1R,5S)-4-(4-(diethylphosphoryl)-3-(methylamino)phenyl)-3-oxo-2,4-diazabicyclo[3.1.0]hexan-2-yl)-2′-(4-fluoro-3,5-dimethylphenyl)-4′-methyl-2′,4′,5′,6′-tetrahydrospiro[cyclopropane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3′-((1R,5S)-4-(4-fluoro-1-methyl-1H-indazol-5-yl)-3-oxo-2,4-diazabicyclo[3.1.0]hexan-2-yl)-2′-(4-fluoro-3,5-dimethylphenyl)-4′-methyl-2′,4′,5′,6′-tetrahydrospiro[cyclobutane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3′-((1S,5R)-4-(4-fluoro-1-methyl-1H-indazol-5-yl)-3-oxo-2,4-diazabicyclo[3.1.0]hexan-2-yl)-2′-(4-fluoro-3,5-dimethylphenyl)-4′-methyl-2′,4′,5′,6′-tetrahydrospiro[cyclobutane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-2-(4-fluoro-3,5-dimethylphenyl)-4-methyl-3-((1S,5R)-4-(1-methyl-1H-indazol-5-yl)-3-oxo-2,4-diazabicyclo[3.1.0]hexan-2-yl)-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-2-(4-fluoro-3,5-dimethylphenyl)-4-methyl-3-((1R,5S)-4-(1-methyl-1H-indazol-5-yl)-3-oxo-2,4-diazabicyclo[3.1.0]hexan-2-yl)-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(2-((S)-3-(1-(4-fluoro-1-methyl-1H-indazol-5-yl)-2-oxo-1,2,5,6-tetrahydropyridin-3-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-(3-(4-fluoro-1-methyl-1H-indazol-5-yl)-2-oxo-2,3-dihydro-1H-imidazol-1-yl)-2-(2-(6-fluoro-3,4-dihydroquinolin-1(2H)-yl)-2-oxoethyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-(3-(4-fluoro-1-methyl-1H-indazol-5-yl)-2-oxo-2,3-dihydro-1H-imidazol-1-yl)-4-methyl-2-(2-oxo-2-(4-azaspiro[2.6]nonan-4-yl)ethyl)-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-(2-(4-azadispiro[2.1.25.33]decan-4-yl)-2-oxoethyl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-(2-((1R,5S)-8-azabicyclo[3.2.1]octan-8-yl)-2-oxoethyl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-(2-((1R,4R)-7-azabicyclo[2.2.1]heptan-7-yl)-2-oxoethyl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-(2-((2S,5R)-2,5-dimethylpyrrolidin-1-yl)-2-oxoethyl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-2-(2-(3,4-dihydroquinolin-1(2H)-yl)-2-oxoethyl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
N-benzyl-N-(cyclopropylmethyl)-2-((S)-3-(3-(4-fluoro-1-methyl-1H-indazol-5-yl)-2-oxo-2,3-dihydro-1H-imidazol-1-yl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-2-yl)acetamide;
3-((1S,2S)-1-(2-((S)-2-(2-(4,4-difluoropiperidin-1-yl)-2-oxoethyl)-3-(3-(4-fluoro-1-methyl-1H-indazol-5-yl)-2-oxo-2,3-dihydro-1H-imidazol-1-yl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-(3-(4-fluoro-1-methyl-1H-indazol-5-yl)-2-oxo-2,3-dihydro-1H-imidazol-1-yl)-4-methyl-2-(2-oxo-2-(4-azaspiro[2.5]octan-4-yl)ethyl)-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
2-((S)-3-(3-(4-fluoro-1-methyl-1H-indazol-5-yl)-2-oxo-2,3-dihydro-1H-imidazol-1-yl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-2-yl)-N-(3-(trifluoromethyl)oxetan-3-yl)acetamide;
N-(bicyclo[2.2.2]octan-1-yl)-2-((S)-3-(3-(4-fluoro-1-methyl-1H-indazol-5-yl)-2-oxo-2,3-dihydro-1H-imidazol-1-yl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-2-yl)acetamide;
3-((1S,2S)-1-(2-((S)-3′-(3-(4-fluoro-1-methyl-1H-indazol-5-yl)-2-oxoimidazolidin-1-yl)-2′-(5-fluoro-4,6-dimethylpyrimidin-2-yl)-4′-methyl-2′,4′,5′,6′-tetrahydrospiro[cyclopropane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3′-(3-(4-fluoro-1-methyl-1H-indazol-5-yl)-2-oxoimidazolidin-1-yl)-1′-(5-fluoro-4,6-dimethylpyrimidin-2-yl)-4′-methyl-1′,4′,5′,6′-tetrahydrospiro[cyclopropane-1,7′-pyrazolo[4,3-c]pyridine]-5′-carbonyl)-5-(tetrahydro-2H-pyran-4-yl)-1H-indol-1-yl)-2-methylcyclopropyl)-1,2,4-oxadiazol-5(4H)-one;
(S)-3-(1-(5-(3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-3-(isochroman-6-yl)-1H-pyrazol-1-yl)cyclopropyl)-1,2,4-oxadiazol-5(4H)-one;
3-(1-(5-((S)-2,2-dimethyltetrahydro-2H-pyran-4-yl)-2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)phenyl)cyclopropyl)-1,2,4-oxadiazol-5(4H)-one;
3-(1-(5-((R)-2,2-dimethyltetrahydro-2H-pyran-4-yl)-2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)phenyl)cyclopropyl)-1,2,4-oxadiazol-5(4H)-one;
(S)-3-(1-(5-(3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-3-((tetrahydro-2H-pyran-4-yl)ethynyl)-1H-pyrazol-1-yl)cyclopropyl)-1,2,4-oxadiazol-5(4H)-one;
(S)-3-(1-(5-(3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-3-(4-(1-methoxycyclobutyl)phenyl)-1H-pyrazol-1-yl)cyclopropyl)-1,2,4-oxadiazol-5(4H)-one;
3-(1-(2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-((3aR,6aR)-hexahydro-1H-cyclopenta[c]furan-5-yl)phenyl)cyclopropyl)-1,2,4-oxadiazol-5(4H)-one;
3-(1-(2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-((3aS,6aS)-hexahydro-1H-cyclopenta[c]furan-5-yl)phenyl)cyclopropyl)-1,2,4-oxadiazol-5(4H)-one;
(S)-3-(1-(2-(3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(2-oxaspiro[3.5]nonan-7-yl)phenyl)cyclopropyl)-1,2,4-oxadiazol-5(4H)-one;
3-(1-(2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-((3aR,6aR)-hexahydro-1H-cyclopenta[c]furan-5-yl)-1H-indol-1-yl)cyclopropyl)-1,2,4-oxadiazol-5(4H)-one;
3-(1-(2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-((3aS,6aS)-hexahydro-1H-cyclopenta[c]furan-5-yl)-1H-indol-1-yl)cyclopropyl)-1,2,4-oxadiazol-5(4H)-one;
3-(1-(2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-((R)-2,5-dioxaspiro[3.5]nonan-7-yl)-1H-indol-1-yl)cyclopropyl)-1,2,4-oxadiazol-5(4H)-one;
3-(1-(2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-((S)-2,5-dioxaspiro[3.5]nonan-7-yl)-1H-indol-1-yl)cyclopropyl)-1,2,4-oxadiazol-5(4H)-one;
3-(1-(2-((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(2-hydroxybicyclo[3.2.1]octan-6-yl)-1H-indol-1-yl)cyclopropyl)-1,2,4-oxadiazol-5(4H)-one;
(S)-3-(1-(2-(3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-4,5,6,7-tetrahydro-2H-pyrazolo[4,3-c]pyridine-5-carbonyl)-5-(4-methoxycyclohexyl)-1H-indol-1-yl)cyclopropyl)-1,2,4-oxadiazol-5(4H)-one;
N-(4-fluoro-1-methyl-1H-indazol-5-yl)-2-((S)-2′-(4-fluoro-3,5-dimethylphenyl)-4′-methyl-5′-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-2′,4′,5′,6′-tetrahydrospiro[cyclopropane-1,7′-pyrazolo[4,3-c]pyridin]-3′-yl)acetamide;
2-((S)-5′-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2′-(4-fluoro-3,5-dimethylphenyl)-4′-methyl-2′,4′,5′,6′-tetrahydrospiro[cyclopropane-1,7′-pyrazolo[4,3-c]pyridin]-3′-yl)-N-(4-fluoro-1-methyl-1H-indazol-5-yl)acetamide;
2-((S)-5′-(5-((S)-2,2-dimethyltetrahydro-2H-pyran-4-yl)-1-((1S,2S)-2-methyl-1-(5-oxo-4,5-dihydro-1,2,4-oxadiazol-3-yl)cyclopropyl)-1H-indole-2-carbonyl)-2′-(4-fluoro-3,5-dimethylphenyl)-4′-methyl-2′,4′,5′,6′-tetrahydrospiro[cyclopropane-1,7′-pyrazolo[4,3-c]pyridin]-3′-yl)-N-(1-methyl-1H-indazol-5-yl)acetamide;
2-((S)-2′-(4-fluoro-3,5-dimethylphenyl)-4′-methyl-5′-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-2′,4′,5′,6′-tetrahydrospiro[cyclopropane-1,7′-pyrazolo[4,3-c]pyridin]-3′-yl)-N-(1-methyl-1H-indazol-5-yl)acetamide;
2-((S)-2-(4-fluoro-3,5-dimethylphenyl)-4,7,7-trimethyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)-N-(1-methyl-1H-indazol-5-yl)acetamide;
N-(4-fluoro-1-methyl-1H-indazol-5-yl)-2-((S)-2-(4-fluoro-3,5-dimethylphenyl)-4,7,7-trimethyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)acetamide;
2,2-difluoro-N-(4-fluoro-1-methyl-1H-indazol-5-yl)-2-((S)-2-(4-fluoro-3,5-dimethylphenyl)-4-methyl-5-(1-((1S,2S)-2-methyl-1-(5-oxo-4,5-dihydro-1,2,4-oxadiazol-3-yl)cyclopropyl)-5-(tetrahydro-2H-pyran-4-yl)-1H-indole-2-carbonyl)-4,5,6,7-tetrahydro-2H-pyrazolo[4,3-c]pyridin-3-yl)acetamide;
3-((1S,2S)-1-(2-((S)-3-(1-(4-fluoro-1-methyl-1H-indazol-5-yl)-5-oxo-1,5-dihydro-4H-1,2,4-triazol-4-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-((S)-1-(4-fluoro-1-methyl-1H-indazol-5-yl)-2-oxopiperidin-3-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-((R)-1-(4-fluoro-1-methyl-1H-indazol-5-yl)-2-oxopiperidin-3-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-((1S,6S)-3-(4-fluoro-1-methyl-1H-indazol-5-yl)-2-oxo-3-azabicyclo[4.1.0]heptan-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-((1R,6R)-3-(4-fluoro-1-methyl-1H-indazol-5-yl)-2-oxo-3-azabicyclo[4.1.0]heptan-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-3-((1R,6R)-3-(4-fluoro-1-methyl-1H-indazol-5-yl)-2-oxo-3-azabicyclo[4.1.0]heptan-1-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-3-((1S,6S)-3-(4-fluoro-1-methyl-1H-indazol-5-yl)-2-oxo-3-azabicyclo[4.1.0]heptan-1-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one 3-((1S,2S)-1-(5-((S)-2,2-dimethyltetrahydro-2H-pyran-4-yl)-2-((S)-3-((1R,6R)-3-(4-fluoro-1-methyl-1H-indazol-5-yl)-2-oxo-3-azabicyclo[4.1.0]heptan-1-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(5-((S)-2,2-dimethyltetrahydro-2H-pyran-4-yl)-2-((S)-3-((1S,6S)-3-(4-fluoro-1-methyl-1H-indazol-5-yl)-2-oxo-3-azabicyclo[4.1.0]heptan-1-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one;
3-((1S,2S)-1-(2-((S)-3-((1R,6R)-3-(4-fluoro-1-methyl-1H-indazol-5-yl)-2-oxo-3-azabicyclo[4.1.0]heptan-1-yl)-2-(4-fluoro-3,5-dimethylphenyl)-4,7,7-trimethyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-((1S,6S)-3-(4-fluoro-1-methyl-1H-indazol-5-yl)-2-oxo-3-azabicyclo[4.1.0]heptan-1-yl)-2-(4-fluoro-3,5-dimethylphenyl)-4,7,7-trimethyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-((1R,6R)-3-(4-fluoro-1-methyl-1H-indazol-5-yl)-2-oxo-3-azabicyclo[4.1.0]heptan-1-yl)-2-(4-fluoro-3,5-dimethylphenyl)-4,7,7-trimethyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-((1S,6S)-3-(4-fluoro-1-methyl-1H-indazol-5-yl)-2-oxo-3-azabicyclo[4.1.0]heptan-1-yl)-2-(4-fluoro-3,5-dimethylphenyl)-4,7,7-trimethyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-(5-(4-fluoro-1-methyl-1H-indazol-5-yl)-1H-pyrazol-3-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((Z)—((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-2,4,6,7-tetrahydro-5H-pyrazolo[4,3-c]pyridin-5-yl)(methylimino)methyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((Z)-(ethylimino)((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-2,4,6,7-tetrahydro-5H-pyrazolo[4,3-c]pyridin-5-yl)methyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((Z)-((cyclopropylmethyl)imino)((S)-3-(3-(4-fluoro-1-methyl-1H-indazol-5-yl)-2-oxo-2,3-dihydro-1H-imidazol-1-yl)-2-(4-fluoro-3,5-dimethylphenyl)-4-methyl-2,4,6,7-tetrahydro-5H-pyrazolo[4,3-c]pyridin-5-yl)methyl)-5-(tetrahydro-2H-pyran-4-yl)-1H-indol-1-yl)-2-methylcyclopropyl)-1,2,4-oxadiazol-5(4H)-one;
3-((1S,2S)-1-(2-((S)-3-(4-(4-fluoro-1-methyl-1H-indazol-5-yl)-1H-pyrazol-1-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one;
(2S,4S)-2,5,12-trihydroxy-7-methoxy-4-(((1S,3R,4aS,9S,9aR,10aS)-9-methoxy-1-methyloctahydro-1H-pyrano[4′,3′:4,5]oxazolo[2,3-c][1,4]oxazin-3-yl)oxy)-6,11-dioxo-N-(pyrrolidin-3-yl)-1,2,3,4,6,11-hexahydrotetracene-2-carboxamide;
3-((1S,2S)-1-(2-((S)-3-(1-(4-fluoro-1-methyl-1H-indazol-5-yl)-1H-pyrazol-4-yl)-2-(4-fluoro-3,5-dimethylphenyl)-4-methyl-4,5,6,7-tetrahydro-2H-pyrazolo[4,3-c]pyridine-5-carbonyl)-5-(tetrahydro-2H-pyran-4-yl)-1H-indol-1-yl)-2-methylcyclopropyl)-1,2,4-oxadiazol-5(4H)-one; and
3-((1S,2S)-1-(5-((S)-2,2-dimethyltetrahydro-2H-pyran-4-yl)-2-((S)-3-(3-(4-fluoro-1-methyl-1H-indazol-5-yl)-2-oxopyridin-1(2H)-yl)-2-(4-fluoro-3,5-dimethylphenyl)-4-methyl-4,5,6,7-tetrahydro-2H-pyrazolo[4,3-c]pyridine-5-carbonyl)-1H-indol-1-yl)-2-methylcyclopropyl)-1,2,4-oxadiazol-5(4H)-one,
or a pharmaceutically acceptable salt thereof.

111. A compound selected from:
3-[(1S,2S)-1-[5-[(4S)-2,2-dimethyltetrahydropyran-4-yl]-2-[(4S)-2-(4-fluoro-3,5-dimethyl-phenyl)-4-methyl-3-[3-(3-methylbenzimidazol-1-ium-5-yl)-2-oxo-imidazol-1-yl]-4,6,7,8-tetrahydropyrazolo[4,3-c]azepine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-4-methyl-3-[3-(1-methylbenzotriazol-5-yl)-2-oxo-imidazol-1-yl]-4,6,7,8-tetrahydropyrazolo[4,3-c]azepin-1-ium-5-carbonyl]indol-1-yl]-2-methyl-cyclopropyl]-4H-1,2,4-oxadiazol-5-one;
3-[(1S,2S)-1-[2-[(4S)-3-[3-(2,3-dimethylimidazo[1,2-a]pyridin-1-ium-6-yl)-2-oxo-imidazol-1-yl]-2-(4-fluoro-3,5-dimethyl-phenyl)-4-methyl-4,6,7,8-tetrahydropyrazolo[4,3-c]azepine-5-carbonyl]-5-[(4S)-2,2-dimethyltetrahydropyran-4-yl]indol-1-yl]-2-methyl-cyclopropyl]-4H-1,2,4-oxadiazol-5-one;
3-[(1S,2S)-1-[2-[(4S)-3-[3-[2-(difluoromethyl)indazol-5-yl]-2-oxo-imidazol-1-yl]-2-(4-fluoro-3,5-dimethyl-phenyl)-4-methyl-4,6,7,8-tetrahydropyrazolo[4,3-c]azepin-1-ium-5-carbonyl]-5-[(4S)-2,2-dimethyltetrahydropyran-4-yl]indol-1-yl]-2-methyl-cyclopropyl]-4H-1,2,4-oxadiazol-5-one;
3-[(1S,2S)-1-[2-[(4S)-3-[3-(2,3-dimethylpyridin-1-ium-4-yl)-2-oxo-imidazol-1-yl]-2-(4-fluoro-3,5-dimethyl-phenyl)-4-methyl-4,6,7,8-tetrahydropyrazolo[4,3-c]azepine-5-carbonyl]-5-[(4S)-2,2-dimethyltetrahydropyran-4-yl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-4-methyl-3-[3-(1-methylisoquinolin-2-ium-6-yl)-2-oxo-imidazol-1-yl]-4,6,7,8-tetrahydropyrazolo[4,3-c]azepine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[3-(4-fluoro-1-methyl-indazol-5-yl)-2-oxo-imidazol-1-yl]-4-methyl-spiro[6,8-dihydro-4H-pyrazolo[4,3-c]azepin-1-ium-7,1′-cyclopropane]-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(5-fluoro-4,6-dimethyl-pyridin-1-ium-2-yl)-3-[3-(4-fluoro-1-methyl-indazol-5-yl)-2-oxo-imidazol-1-yl]-4-methyl-4,6,7,8-tetrahydropyrazolo[4,3-c]azepine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-4-methyl-3-[2-oxo-3-[2-(2,2,2-trifluoroethyl)indazol-5-yl]imidazol-1-yl]-4,6,7,8-tetrahydropyrazolo[4,3-c]azepin-1-ium-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-4-methyl-3-[3-(1-methylindazol-5-yl)-2-oxo-imidazol-1-yl]-4,6,7,8-tetrahydropyrazolo[4,3-c]azepin-1-ium-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-4-methyl-3-[2-oxo-3-[1-(trideuteriomethyl)indazol-5-yl]imidazol-1-yl]-4,6,7,8-tetrahydropyrazolo[4,3-c]azepin-1-ium-5-carbonyl]indol-1-yl]-2-methyl-cyclopropyl]-4H-1,2,4-oxadiazol-5-one;
3-[(1S,2S)-1-[2-[(4S)-3-[3-(1-cyclopropylindazol-5-yl)-2-oxo-imidazol-1-yl]-2-(4-fluoro-3,5-dimethyl-phenyl)-4-methyl-4,6,7,8-tetrahydropyrazolo[4,3-c]azepin-1-ium-5-carbonyl]-5-[(4S)-2,2-dimethyltetrahydropyran-4-yl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[3-(4-fluoro-1-methyl-indazol-5-yl)-2-oxo-imidazol-1-yl]-4,7,7-trimethyl-6,8-dihydro-4H-pyrazolo[4,3-c]azepine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-4-methyl-3-[2-oxo-3-[1-(2,2,2-trifluoroethyl)indazol-5-yl]imidazol-1-yl]-4,6,7,8-tetrahydropyrazolo[4,3-c]azepin-1-ium-5-carbonyl]indol-1-yl]-2-methyl-cyclopropyl]-4H-1,2,4-oxadiazol-5-one;
3-[(1S,2S)-1-[5-(2,2-dimethylmorpholin-4-ium-4-yl)-2-[(4S)-4-ethyl-2-(4-fluoro-3,5-dimethyl-phenyl)-3-[3-(4-fluoro-1-methyl-indazol-5-yl)-2-oxo-imidazol-1-yl]-4,6,7,8-tetrahydropyrazolo[4,3-c]azepine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-3-[3-(4-fluoro-1-methyl-indazol-5-yl)-2-oxo-imidazol-1-yl]-4-methyl-2-[5-(trifluoromethyl)-3-pyridyl]-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[3-(4-fluoro-1-methyl-indazol-5-yl)-2-oxo-imidazol-1-yl]-4-methyl-spiro[4,6-dihydropyrazolo[4,3-c]pyridine-7,4′-tetrahydropyran]-5-carbonyl]indol-1-yl]-2-methyl-cyclopropyl]-4H-1,2,4-oxadiazol-5-one;
calcium 3-[(1S,2S)-1-[5-[(4S)-2,2-dimethyltetrahydropyran-4-yl]-2-[(4S)-2-(4-fluoro-3,5-dimethyl-phenyl)-4-methyl-3-[1-(2-methyl-6-quinolyl)-2-oxo-3-pyridyl]-6,7-dihydro-4H-pyrazolo[4,3-c]pyridine-5-carbonyl]indol-1-yl]-2-methyl-cyclopropyl]-1-oxa-2-aza-4-azanidacyclopent-2-en-5-one;
3-[(1S,2S)-1-[2-[(4S)-2-(4-fluoro-3,5-dimethyl-phenyl)-4-methyl-3-[1-(2-methyl-6-quinolyl)-2-oxo-3-pyridyl]-6,7-dihydro-4H-pyrazolo[4,3-c]pyridine-5-carbonyl]-5-[(2S)-2-methylmorpholin-4-yl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-4-methyl-3-[1-(2-methyl-6-quinolyl)-6-oxo-pyrimidin-5-yl]-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[1-(4-fluoro-1-methyl-indazol-5-yl)-6-oxo-pyrimidin-5-yl]-4-methyl-4,6,7,8-tetrahydropyrazolo[4,3-c]azepin-1-ium-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-4-methyl-3-[1-(1-methylisoquinolin-2-ium-6-yl)-2-oxo-3-pyridyl]-4,6,7,8-tetrahydropyrazolo[4,3-c]azepine-5-carbonyl]indol-1-yl]-2-methyl-cyclopropyl]-4H-1,2,4-oxadiazol-5-one;
3-[(1S,2S)-1-[2-[(4S)-3-[1-[2-(cyclopropoxy)pyridin-1-ium-4-yl]-2-oxo-3-pyridyl]-2-(4-fluoro-3,5-dimethyl-phenyl)-4-methyl-4,6,7,8-tetrahydropyrazolo[4,3-c]azepine-5-carbonyl]-5-[(4S)-2,2-dimethyltetrahydropyran-4-yl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[1-(4-fluoro-1-methyl-indazol-5-yl)-2-oxo-3-pyridyl]-4-methyl-4,6,7,8-tetrahydropyrazolo[4,3-c]azepin-1-ium-5-carbonyl]indol-1-yl]-2-methyl-cyclopropyl]-4H-1,2,4-oxadiazol-5-one;
3-[(1S,2S)-1-[2-[(4S)-2-(4-fluoro-3,5-dimethyl-phenyl)-3-[1-(4-fluoro-1-methyl-indazol-2-ium-5-yl)-2-oxo-3-pyridyl]-4-methyl-spiro[4,6-dihydropyrazolo[4,3-c]pyridine-7,1′-cyclobutane]-5-carbonyl]-5-tetrahydropyran-4-yl-indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[1-(5-fluoro-2-methyl-6-quinolyl)-2-oxo-3-pyridyl]-4-methyl-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[1-(4-fluoro-1-methyl-indazol-5-yl)-2-oxo-3-pyridyl]-4-methyl-1′-(2,2,2-trifluoroethyl)spiro[4,6-dihydropyrazolo[4,3-c]pyridine-7,3′-azetidin-1-ium]-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[1-(5-fluoro-1-methyl-isoquinolin-2-ium-6-yl)-2-oxo-3-pyridyl]-4-methyl-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[1-(4-fluoro-1-methyl-indazol-2-ium-5-yl)-2-oxo-3-pyridyl]-4-methyl-spiro[4,6-dihydropyrazolo[4,3-c]pyridine-7,1′-cyclobutane]-5-carbonyl]indol-1-yl]-2-methyl-cyclopropyl]-4H-1,2,4-oxadiazol-5-one;
N-(1-cyclopropyl-4-fluoro-indazol-5-yl)-2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-spiro[6,8-dihydro-4H-pyrazolo[4,3-c]azepin-1-ium-7,1′-cyclopropane]-3-yl]acetamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-spiro[6,8-dihydro-4H-pyrazolo[4,3-c]azepin-1-ium-7,1′-cyclopropane]-3-yl]-N-(1-methylindazol-5-yl)acetamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-spiro[6,8-dihydro-4H-pyrazolo[4,3-c]azepin-1-ium-7,1′-cyclopropane]-3-yl]-N-(6-fluoro-1-methyl-indazol-5-yl)acetamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-spiro[6,8-dihydro-4H-pyrazolo[4,3-c]azepin-1-ium-7,1′-cyclopropane]-3-yl]-N-(4-fluoro-1-methyl-indazol-5-yl)acetamide;
N-(1-methylindazol-5-yl)-2-[(4S)-4,7,7-trimethyl-5-[1-[(1S,2S)-2-methyl-1-(5-oxo-4H-1,2,4-oxadiazol-3-yl)cyclopropyl]-5-tetrahydropyran-4-yl-indole-2-carbonyl]-2-[1-methyl-5-(trifluoromethyl)pyrazol-3-yl]-4,6-dihydropyrazolo[4,3-c]pyridin-1-ium-3-yl]acetamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-4,7,7-trimethyl-2-[1-methyl-5-(trifluoromethyl)pyrazol-3-yl]-4,6-dihydropyrazolo[4,3-c]pyridin-1-ium-3-yl]-N-(1-methylindazol-5-yl)acetamide;
2-[(4S)-2-(4-fluoro-3,5-dimethyl-phenyl)-4-methyl-5-[1-[(1S,2S)-2-methyl-1-(5-oxo-4H-1,2,4-oxadiazol-3-yl)cyclopropyl]-5-tetrahydropyran-4-yl-indole-2-carbonyl]-6,7-dihydro-4H-pyrazolo[4,3-c]pyridin-3-yl]-N-(2-methylquinolin-1-ium-6-yl)acetamide;
2-[(4S)-2-(4-fluoro-3,5-dimethyl-phenyl)-4,7,7-trimethyl-5-[5-[(2S)-2-methylmorpholin-4-yl]-1-[(1S,2S)-2-methyl-1-(5-oxo-4H-1,2,4-oxadiazol-3-yl)cyclopropyl]indole-2-carbonyl]-4,6-dihydropyrazolo[4,3-c]pyridin-3-yl]-N-(2-methylquinolin-1-ium-6-yl)acetamide;
2-[(4S)-2-(4-fluoro-3,5-dimethyl-phenyl)-4,7,7-trimethyl-5-[5-[(2S)-2-methylmorpholin-4-ium-4-yl]-1-[(1S,2S)-2-methyl-1-(5-oxo-4H-1,2,4-oxadiazol-3-yl)cyclopropyl]indole-2-carbonyl]-4,6-dihydropyrazolo[4,3-c]pyridin-3-yl]-N-(1-methylindazol-5-yl)acetamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4,7,7-trimethyl-4,6-dihydropyrazolo[4,3-c]pyridin-1-ium-3-yl]-N-(4-fluoro-1-methyl-indazol-5-yl)acetamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4,7,7-trimethyl-4,6-dihydropyrazolo[4,3-c]pyridin-3-yl]-N-(2-methylquinolin-1-ium-6-yl)acetamide;
2-[(4S)-2-(4-fluoro-3,5-dimethyl-phenyl)-4-methyl-5-[1-[(1S,2S)-2-methyl-1-(5-oxo-4H-1,2,4-oxadiazol-3-yl)cyclopropyl]-5-tetrahydropyran-4-yl-indole-2-carbonyl]-6,7-dihydro-4H-pyrazolo[4,3-c]pyridin-1-ium-3-yl]-N-(4-methoxy-1-bicyclo[2.2.2]octanyl)acetamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4,7,7-trimethyl-4,6-dihydropyrazolo[4,3-c]pyridin-1-ium-3-yl]-N-(1-methylindazol-5-yl)acetamide;
2-[(4S)-2-(4-fluoro-3,5-dimethyl-phenyl)-4-methyl-5-[5-[(2S)-2-methylmorpholin-4-ium-4-yl]-1-[(1S,2S)-2-methyl-1-(5-oxo-4H-1,2,4-oxadiazol-3-yl)cyclopropyl]indole-2-carbonyl]-6,7-dihydro-4H-pyrazolo[4,3-c]pyridin-3-yl]-N-(4-fluoro-1-methyl-indazol-5-yl)acetamide;
2-[(4S)-2-(4-fluoro-3,5-dimethyl-phenyl)-4-methyl-5-[5-[(2R)-2-methylmorpholin-4-ium-4-yl]-1-[(1S,2S)-2-methyl-1-(5-oxo-4H-1,2,4-oxadiazol-3-yl)cyclopropyl]indole-2-carbonyl]-6,7-dihydro-4H-pyrazolo[4,3-c]pyridin-3-yl]-N-(4-fluoro-1-methyl-indazol-5-yl)acetamide;
N-[4-(cyclopropoxy)cyclohexyl]-2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4,7,7-trimethyl-4,6-dihydropyrazolo[4,3-c]pyridin-1-ium-3-yl]acetamide;
3-[(1S,2S)-1-[5-[(4S)-2,2-dimethyltetrahydropyran-4-yl]-2-[(4S)-2-(4-fluoro-3,5-dimethyl-phenyl)-3-[4-(4-fluoro-1-methyl-indazol-5-yl)-3-oxo-2,4-diazabicyclo[3.1.0]hexan-2-yl]-4-methyl-4,6,7,8-tetrahydropyrazolo[4,3-c]azepin-1-ium-5-carbonyl]indol-1-yl]-2-methyl-cyclopropyl]-4H-1,2,4-oxadiazol-5-one;
3-[1-[2-[(4S)-2-(4-fluoro-3,5-dimethyl-phenyl)-3-[3-(4-fluoro-1-methyl-indazol-5-yl)-2-oxo-imidazol-1-yl]-4-methyl-6,7-dihydro-4H-pyrazolo[4,3-c]pyridine-5-carbonyl]-5-[(1S,5R)-6-hydroxy-2-bicyclo[3.2.1]octanyl]indol-1-yl]cyclopropyl]-4H-1,2,4-oxadiazol-5-one;
3-[1-[2-[(4S)-2-(4-fluoro-3,5-dimethyl-phenyl)-3-[3-(4-fluoro-1-methyl-indazol-5-yl)-2-oxo-imidazol-1-yl]-4-methyl-6,7-dihydro-4H-pyrazolo[4,3-c]pyridine-5-carbonyl]-5-[(1S,5R)-6-hydroxy-2-bicyclo[3.2.1]octanyl]indol-1-yl]cyclopropyl]-4H-1,2,4-oxadiazol-5-one;
3-[(1S,2S)-1-[2-[(4S)-2-(4-fluoro-3,5-dimethyl-phenyl)-3-[3-(4-fluoro-1-methyl-indazol-5-yl)-2-oxo-imidazol-1-yl]-4-methyl-4,6,7,8-tetrahydropyrazolo[4,3-c]azepine-5-carbonyl]-5-(4-oxa-7-azoniaspiro[2.5]octan-7-yl)indol-1-yl]-2-methyl-cyclopropyl]-4H-1,2,4-oxadiazol-5-one;
3-[(1S,2S)-1-[2-[(4S)-2-(4-fluoro-3,5-dimethyl-phenyl)-3-[3-(4-fluoro-1-methyl-indazol-5-yl)-2-oxo-imidazol-1-yl]-4-methyl-4,6,7,8-tetrahydropyrazolo[4,3-c]azepine-5-carbonyl]-5-[(2S)-2-methylmorpholin-4-ium-4-yl]indol-1-yl]-2-methyl-cyclopropyl]-4H-1,2,4-oxadiazol-5-one;
3-[(1S,2S)-1-[5-(2,2-dimethylmorpholin-4-ium-4-yl)-2-[(4S)-2-(4-fluoro-3,5-dimethyl-phenyl)-3-[3-(4-fluoro-1-methyl-indazol-5-yl)-2-oxo-imidazol-1-yl]-4-methyl-4,6,7,8-tetrahydropyrazolo[4,3-c]azepine-5-carbonyl]indol-1-yl]-2-methyl-cyclopropyl]-4H-1,2,4-oxadiazol-5-one;
3-[1-[3-(4,4-difluoroisochroman-6-yl)-5-[(4S)-2-(4-fluoro-3,5-dimethyl-phenyl)-3-[3-(4-fluoro-1-methyl-indazol-5-yl)-2-oxo-imidazol-1-yl]-4-methyl-6,7-dihydro-4H-pyrazolo[4,3-c]pyridine-5-carbonyl]pyrazol-1-yl]cyclopropyl]-4H-1,2,4-oxadiazol-5-one;
3-[(1S,2S)-1-[3-(1,7-dimethylindazol-2-ium-6-yl)-5-[(4S)-2-(4-fluoro-3,5-dimethyl-phenyl)-3-[3-(4-fluoro-1-methyl-indazol-5-yl)-2-oxo-imidazol-1-yl]-4-methyl-6,7-dihydro-4H-pyrazolo[4,3-c]pyridine-5-carbonyl]pyrazol-1-yl]-2-methyl-cyclopropyl]-4H-1,2,4-oxadiazol-5-one;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-2,2-difluoro-N-(4-fluoro-1-methyl-indazol-5-yl)acetamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-1-ium-3-yl]-N-(4-fluoro-1-methyl-indazol-5-yl)acetamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-1′-(2,2,2-trifluoroethyl)spiro[4,6-dihydropyrazolo[4,3-c]pyridine-7,3′-azetidin-1-ium]-3-yl]-N-(4-fluoro-1-methyl-indazol-5-yl)acetamide;
3-[(1S,2S)-1-[5-[(4S)-2,2-dimethyltetrahydropyran-4-yl]-2-[(4S)-2-(4-fluoro-3,5-dimethyl-phenyl)-3-[(1S,6S)-3-(4-fluoro-1-methyl-indazol-5-yl)-2-oxo-3-azabicyclo[4.1.0]heptan-1-yl]-4-methyl-spiro[4,6-dihydropyrazolo[4,3-c]pyridine-7,1′-cyclopropane]-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[(1R,6R)-3-(4-fluoro-1-methyl-indazol-5-yl)-2-oxo-3-azabicyclo[4.1.0]heptan-1-yl]-4-methyl-spiro[4,6-dihydropyrazolo[4,3-c]pyridine-7,1′-cyclopropane]-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[3-(4-fluoro-1-methyl-indazol-5-yl)-2-oxo-3-azabicyclo[4.1.0]heptan-1-yl]-4-methyl-4,6,7,8-tetrahydropyrazolo[4,3-c]azepine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[3-(4-fluoro-1-methyl-indazol-5-yl)-2-oxo-3-azabicyclo[4.1.0]heptan-1-yl]-4-methyl-4,6,7,8-tetrahydropyrazolo[4,3-c]azepine-5-carbonyl]indol-1-yl]-2-methyl-cyclopropyl]-4H-1,2,4-oxadiazol-5-one;
1-[(4S)-2-(4-fluoro-3,5-dimethyl-phenyl)-4-methyl-5-[1-[(1S,2S)-2-methyl-1-(5-oxo-4H-1,2,4-oxadiazol-3-yl)cyclopropyl]-5-tetrahydropyran-4-yl-indole-2-carbonyl]-6,7-dihydro-4H-pyrazolo[4,3-c]pyridin-3-yl]-N-(1-methylindazol-5-yl)cyclopropanecarboxamide;
3-[(1S,2S)-1-[2-[(4S)-2-(4-fluoro-3,5-dimethyl-phenyl)-4-methyl-3-[1-(1-methylindazol-5-yl)-2-oxo-pyrrolidin-3-yl]-6,7-dihydro-4H-pyrazolo[4,3-c]pyridine-5-carbonyl]-5-tetrahydropyran-4-yl-indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-4-methyl-3-[(1S,6S)-3-(2-methyl-6-quinolyl)-2-oxo-3-azabicyclo[4.1.0]heptan-1-yl]-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-4-methyl-3-[(1S,6S)-3-(1-methyl-6-isoquinolyl)-2-oxo-3-azabicyclo[4.1.0]heptan-1-yl]-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[2-[(4S)-3-[(1S,6S)-3-[2-(cyclopropoxy)-4-pyridyl]-2-oxo-3-azabicyclo[4.1.0]heptan-1-yl]-2-(4-fluoro-3,5-dimethyl-phenyl)-4-methyl-6,7-dihydro-4H-pyrazolo[4,3-c]pyridine-5-carbonyl]-5-[(4S)-2,2-dimethyltetrahydropyran-4-yl]indol-1-yl]-2-methyl-cyclopropyl]-4H-1,2,4-oxadiazol-5-one;
3-[(1S,2S)-1-[2-[(4S)-3-[1-[4-(cyclopropoxy)cyclohexyl]-6-oxo-pyrimidin-5-yl]-2-(4-fluoro-3,5-dimethyl-phenyl)-4-methyl-6,7-dihydro-4H-pyrazolo[4,3-c]pyridine-5-carbonyl]-5-[(4S)-2,2-dimethyltetrahydropyran-4-yl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[1-(4-methoxycyclohexyl)-6-oxo-pyrimidin-5-yl]-4-methyl-4,6,7,8-tetrahydropyrazolo[4,3-c]azepin-1-ium-5-carbonyl]indol-1-yl]-2-methyl-cyclopropyl]-4H-1,2,4-oxadiazol-5-one;
3-[(1S,2S)-1-[2-[(4S)-1′-(2,2-dimethylpropanoyl)-2-(4-fluoro-3,5-dimethyl-phenyl)-3-[3-(4-fluoro-1-methyl-indazol-5-yl)-2-oxo-imidazol-1-yl]-4-methyl-spiro[4,6-dihydropyrazolo[4,3-c]pyridine-7,3′-azetidine]-5-carbonyl]-5-tetrahydropyran-4-yl-indol-1-yl]-2-methyl-cyclopropyl]-4H-1,2,4-oxadiazol-5-one;
3-[(1S,2S)-1-[2-[(4S)-1′-(2,2-difluoroacetyl)-2-(4-fluoro-3,5-dimethyl-phenyl)-3-[3-(4-fluoro-1-methyl-indazol-5-yl)-2-oxo-imidazol-1-yl]-4-methyl-spiro[4,6-dihydropyrazolo[4,3-c]pyridine-7,3′-azetidine]-5-carbonyl]-5-tetrahydropyran-4-yl-indol-1-yl]-2-methyl-cyclopropyl]-4H-1,2,4-oxadiazol-5-one;
3-[(1S,2S)-1-[2-[(4S)-1′-(2,2-difluoropropanoyl)-2-(4-fluoro-3,5-dimethyl-phenyl)-3-[3-(4-fluoro-1-methyl-indazol-5-yl)-2-oxo-imidazol-1-yl]-4-methyl-spiro[4,6-dihydropyrazolo[4,3-c]pyridine-7,3′-azetidine]-5-carbonyl]-5-[(4S)-2,2-dimethyltetrahydropyran-4-yl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[3-(4-fluoro-1-methyl-indazol-5-yl)-2-oxo-imidazol-1-yl]-4-methyl-1′-(2,2,3,3-tetrafluoropropanoyl)spiro[4,6-dihydropyrazolo[4,3-c]pyridine-7,3′-azetidine]-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[3-(4-fluoro-1-methyl-indazol-5-yl)-2-oxo-imidazol-1-yl]-4-methyl-1′-(2,2,2-trifluoroacetyl)spiro[4,6-dihydropyrazolo[4,3-c]pyridine-7,3′-azetidine]-5-carbonyl]indol-1-yl]-2-methyl-cyclopropyl]-4H-1,2,4-oxadiazol-5-one;
3-[(1S,2S)-1-[2-[(4S)-1′-acetyl-2-(4-fluoro-3,5-dimethyl-phenyl)-3-[3-(4-fluoro-1-methyl-indazol-5-yl)-2-oxo-imidazol-1-yl]-4-methyl-spiro[4,6-dihydropyrazolo[4,3-c]pyridine-7,3′-azetidine]-5-carbonyl]-5-tetrahydropyran-4-yl-indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[3-(4-fluoro-1-methyl-indazol-5-yl)-2-oxo-imidazol-1-yl]-4-methyl-1′-(2,2,2-trifluoroethyl)spiro[4,6-dihydropyrazolo[4,3-c]pyridine-7,3′-azetidin-1-ium]-5-carbonyl]indol-1-yl]-2-methyl-cyclopropyl]-4H-1,2,4-oxadiazol-5-one;
3-[(1S,2S)-1-[2-[(4S)-2-(4-fluoro-3,5-dimethyl-phenyl)-3-[3-(4-fluoro-1-methyl-indazol-5-yl)-2-oxo-imidazol-1-yl]-4-methyl-1′-(2,2,2-trifluoroethyl)spiro[4,6-dihydropyrazolo[4,3-c]pyridine-7,3′-azetidine]-5-carbonyl]-5-tetrahydropyran-4-yl-indol-1-yl]-2-methyl-cyclopropyl]-4H-1,2,4-oxadiazol-5-one;
3-[(1S,2S)-1-[2-[(4S)-1′-(2,2-difluoroethyl)-2-(4-fluoro-3,5-dimethyl-phenyl)-3-[3-(4-fluoro-1-methyl-indazol-5-yl)-2-oxo-imidazol-1-yl]-4-methyl-spiro[4,6-dihydropyrazolo[4,3-c]pyridine-7,3′-azetidin-1-ium]-5-carbonyl]-5-tetrahydropyran-4-yl-indol-1-yl]-2-methyl-cyclopropyl]-4H-1,2,4-oxadiazol-5-one;
3-[(1S,2S)-1-[2-[(4S)-2-(4-fluoro-3,5-dimethyl-phenyl)-3-[3-(4-fluoro-1-methyl-indazol-5-yl)-2-oxo-imidazol-1-yl]-1′-[2-[2-[2-(2-methoxyethoxy)ethoxy]ethoxy]ethyl]-4-methyl-spiro[4,6-dihydropyrazolo[4,3-c]pyridine-7,3′-azetidin-1-ium]-5-carbonyl]-5-tetrahydropyran-4-yl-indol-1-yl]-2-methyl-cyclopropyl]-4H-1,2,4-oxadiazol-5-one;
3-[(1S,2S)-1-[2-[(4S)-1′-benzyl-2-(4-fluoro-3,5-dimethyl-phenyl)-3-[3-(4-fluoro-1-methyl-indazol-5-yl)-2-oxo-imidazol-1-yl]-4-methyl-spiro[4,6-dihydropyrazolo[4,3-c]pyridine-7,3′-azetidin-1-ium]-5-carbonyl]-5-tetrahydropyran-4-yl-indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[3-(4-fluoro-1-methyl-indazol-5-yl)-2-oxo-imidazol-1-yl]-1′-(2-hydroxy-2-methyl-propyl)-4-methyl-spiro[4,6-dihydropyrazolo[4,3-c]pyridine-7,3′-azetidin-1-ium]-5-carbonyl]indol-1-yl]-2-methyl-cyclopropyl]-4H-1,2,4-oxadiazol-5-one;
3-[(1S,2S)-1-[2-[(4S)-1′-cyclohexyl-2-(4-fluoro-3,5-dimethyl-phenyl)-3-[3-(4-fluoro-1-methyl-indazol-5-yl)-2-oxo-imidazol-1-yl]-4-methyl-spiro[4,6-dihydropyrazolo[4,3-c]pyridine-7,3′-azetidin-1-ium]-5-carbonyl]-5-tetrahydropyran-4-yl-indol-1-yl]-2-methyl-cyclopropyl]-4H-1,2,4-oxadiazol-5-one;
3-[(1S,2S)-1-[2-[(4S)-1′-(cyclopropylmethyl)-2-(4-fluoro-3,5-dimethyl-phenyl)-3-[3-(4-fluoro-1-methyl-indazol-5-yl)-2-oxo-imidazol-1-yl]-4-methyl-spiro[4,6-dihydropyrazolo[4,3-c]pyridine-7,3′-azetidin-1-ium]-5-carbonyl]-5-tetrahydropyran-4-yl-indol-1-yl]-2-methyl-cyclopropyl]-4H-1,2,4-oxadiazol-5-one;
3-[(1S,2S)-1-[2-[(4S)-2-(4-fluoro-3,5-dimethyl-phenyl)-3-[3-(4-fluoro-1-methyl-indazol-5-yl)-2-oxo-imidazol-1-yl]-4-methyl-1′-methylsulfonyl-spiro[4,6-dihydropyrazolo[4,3-c]pyridine-7,3′-azetidine]-5-carbonyl]-5-tetrahydropyran-4-yl-indol-1-yl]-2-methyl-cyclopropyl]-4H-1,2,4-oxadiazol-5-one;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-4-methyl-2-[5-(trifluoromethyl)-3-pyridyl]-6,7-dihydro-4H-pyrazolo[4,3-c]pyridin-3-yl]-N-(4-fluoro-1-methyl-indazol-5-yl)acetamide;
2-[(4S)-2-(3-cyclopropyl-4-fluoro-phenyl)-5-[7-[(4S)-2,2-dimethyltetrahydropyran-4-yl]-3-[1-(5-oxo-4H-1,2,4-oxadiazol-3-yl)cyclopropyl]indolizine-2-carbonyl]-4-methyl-6,7-dihydro-4H-pyrazolo[4,3-c]pyridin-3-yl]-N-(4-fluoro-1-methyl-indazol-5-yl)acetamide;
3-[1-[2-[(4S)-2-(3-cyclopropyl-4-fluoro-phenyl)-4-methyl-3-[1-(1-methylindazol-5-yl)-2-oxo-3-pyridyl]-6,7-dihydro-4H-pyrazolo[4,3-c]pyridine-5-carbonyl]-7-[(4S)-2,2-dimethyltetrahydropyran-4-yl]indolizin-3-yl]cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-4-methyl-3-[5-(3-methylimidazo[1,5-a]pyridin-7-yl)-1H-1,2,4-triazol-4-ium-3-yl]-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[5-(4-fluoro-1-methyl-indazol-5-yl)-1H-1,2,4-triazol-4-ium-3-yl]-4-methyl-4,6,7,8-tetrahydropyrazolo[4,3-c]azepine-5-carbonyl]indol-1-yl]-2-methyl-cyclopropyl]-4H-1,2,4-oxadiazol-5-one;
3-[(1S,2S)-1-[2-[(4S)-3-[5-(2,3-dimethylimidazo[1,2-a]pyridin-6-yl)-1H-1,2,4-triazol-4-ium-3-yl]-2-(4-fluoro-3,5-dimethyl-phenyl)-4-methyl-6,7-dihydro-4H-pyrazolo[4,3-c]pyridine-5-carbonyl]-5-[(4S)-2,2-dimethyltetrahydropyran-4-yl]indol-1-yl]-2-methyl-cyclopropyl]-4H-1,2,4-oxadiazol-5-one;
1-[(4S)-2-(4-fluoro-3,5-dimethyl-phenyl)-4-methyl-5-[1-[(1S,2S)-2-methyl-1-(5-oxo-4H-1,2,4-oxadiazol-3-yl)cyclopropyl]-5-tetrahydropyran-4-yl-indole-2-carbonyl]-6,7-dihydro-4H-pyrazolo[4,3-c]pyridin-3-yl]-N-(4-fluoro-1-methyl-indazol-5-yl)cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-1-ium-3-yl]-N-(1-methylindazol-5-yl)cyclopropanecarboxamide;
N-[2-(cyclopropoxy)pyridin-1-ium-4-yl]-1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]cyclopropanecarboxamide;
N-[4-(cyclopropoxy)cyclohexyl]-1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-1-ium-3-yl]cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-1-ium-3-yl]-N-(2-fluorophenyl)cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-N-(2-fluorophenyl)cyclopropanecarboxamide;
1-[(4S)-2-(4-fluoro-3,5-dimethyl-phenyl)-4-methyl-5-[5-[(2S)-2-methylmorpholin-4-ium-4-yl]-1-[(1S,2S)-2-methyl-1-(5-oxo-4H-1,2,4-oxadiazol-3-yl)cyclopropyl]indole-2-carbonyl]-6,7-dihydro-4H-pyrazolo[4,3-c]pyridin-3-yl]-N-(4-fluoro-1-methyl-indazol-5-yl)cyclopropanecarboxamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-1-ium-3-yl]-N-(4-fluoro-1-methyl-indazol-5-yl)propanamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-1-ium-3-yl]-N-(4-fluoro-1-methyl-indazol-5-yl)propanamide;
N-(1-methylindazol-5-yl)-2-[rac-(4S)-5-[5-(2,2-dimethyltetrahydropyran-4-yl)-1-[rac-(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]propanamide;
N-(1-methylindazol-5-yl)-2-[rac-(4S)-5-[5-(2,2-dimethyltetrahydropyran-4-yl)-1-[rac-(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]propanamide;
N-(2-fluorophenyl)-2-[rac-(4S)-5-[5-(2,2-dimethyltetrahydropyran-4-yl)-1-[rac-(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]propanamide;
N-(2-fluorophenyl)-2-[rac-(4S)-5-[5-(2,2-dimethyltetrahydropyran-4-yl)-1-[rac-(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]propanamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-1-ium-3-yl]-N-(4-methoxy-1-bicyclo[2.2.2]octanyl)propanamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-1-ium-3-yl]-N-(4-methoxy-1-bicyclo[2.2.2]octanyl)propanamide;
N-[4-(cyclopropoxy)cyclohexyl]-2-[rac-(4S)-5-[5-(2,2-dimethyltetrahydropyran-4-yl)-1-[rac-(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]propanamide;
N-[4-(cyclopropoxy)cyclohexyl]-2-[rac-(4S)-5-[5-(2,2-dimethyltetrahydropyran-4-yl)-1-[rac-(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]propanamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-3-yl]-N-(4-fluoro-1-methyl-indazol-5-yl)propanamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-3-yl]-N-(4-fluoro-1-methyl-indazol-5-yl)propanamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-1-ium-3-yl]-N-(4-fluoro-1-methyl-indazol-5-yl)cyclopropanecarboxamide;
N-(1,3-benzothiazol-3-ium-6-yl)-1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-3-yl]cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-1-ium-3-yl]-N-(2-methylindazol-5-yl)cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-1-ium-3-yl]-N-(1-methylindazol-5-yl)cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-1-ium-3-yl]-N-[1-(trideuteriomethyl)indazol-5-yl]cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-1-ium-3-yl]-N-(6-fluoro-1-methyl-indazol-5-yl)cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-1-ium-3-yl]-N-(1-ethylindazol-5-yl)cyclopropanecarboxamide;
N-(1-cyclopropyl-4-fluoro-indazol-5-yl)-1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-1-ium-3-yl]cyclopropanecarboxamide;
N-(1-cyclopropylindazol-5-yl)-1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-1-ium-3-yl]cyclopropanecarboxamide;
1-[(4S)-2-(4-fluoro-3,5-dimethyl-phenyl)-4-methyl-5-[1-[(1S,2S)-2-methyl-1-(5-oxo-4H-1,2,4-oxadiazol-3-yl)cyclopropyl]-5-tetrahydropyran-4-yl-indole-2-carbonyl]-4,6,7,8-tetrahydropyrazolo[4,3-c]azepin-1-ium-3-yl]-N-(4-fluoro-1-methyl-indazol-5-yl)cyclopropanecarboxamide;
N-(1-cyclopropyl-4-fluoro-indazol-5-yl)-1-[(4S)-2-(4-fluoro-3,5-dimethyl-phenyl)-4-methyl-5-[1-[(1S,2S)-2-methyl-1-(5-oxo-4H-1,2,4-oxadiazol-3-yl)cyclopropyl]-5-tetrahydropyran-4-yl-indole-2-carbonyl]-4,6,7,8-tetrahydropyrazolo[4,3-c]azepin-1-ium-3-yl]cyclopropanecarboxamide;
N-[4-(cyclopropoxy)cyclohexyl]-1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-1-ium-3-yl]cyclopropanecarboxamide;
N-(1-cyclopropyl-4-fluoro-indazol-5-yl)-1-[(4S)-2-(4-fluoro-3,5-dimethyl-phenyl)-4-methyl-5-[5-[(2S)-2-methylmorpholin-4-ium-4-yl]-1-[(1S,2S)-2-methyl-1-(5-oxo-4H-1,2,4-oxadiazol-3-yl)cyclopropyl]indole-2-carbonyl]-4,6,7,8-tetrahydropyrazolo[4,3-c]azepin-3-yl]cyclopropanecarboxamide;
1-[(4S)-2-(4-fluoro-3,5-dimethyl-phenyl)-4-methyl-5-[5-[(2S)-2-methylmorpholin-4-ium-4-yl]-1-[(1S,2S)-2-methyl-1-(5-oxo-4H-1,2,4-oxadiazol-3-yl)cyclopropyl]indole-2-carbonyl]-4,6,7,8-tetrahydropyrazolo[4,3-c]azepin-3-yl]-N-(4-fluoro-1-methyl-indazol-5-yl)cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-1-ium-3-yl]-N-[1-(2,2,2-trifluoroethyl)indazol-5-yl]cyclopropanecarboxamide;
N-(1-cyclopropyl-4-fluoro-indazol-5-yl)-1-[(4S)-2-(4-fluoro-3,5-dimethyl-phenyl)-4-methyl-5-[5-[(2S)-2-methylmorpholin-4-ium-4-yl]-1-[(1S,2S)-2-methyl-1-(5-oxo-4H-1,2,4-oxadiazol-3-yl)cyclopropyl]indole-2-carbonyl]spiro[6,8-dihydro-4H-pyrazolo[4,3-c]azepine-7,1′-cyclopropane]-3-yl]cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-3-yl]-N-isoquinolin-2-ium-6-yl-cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-3-yl]-N-(1-methylisoquinolin-2-ium-6-yl)cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-3-yl]-N-(5,6,7,8-tetrahydroquinolin-1-ium-6-yl)cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-3-yl]-N-(5,6,7,8-tetrahydroisoquinolin-7-yl)cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-3-yl]-N-(5,6,7,8-tetrahydroisoquinolin-7-yl)cyclopropanecarboxamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-N-(4-fluoro-1-methyl-indazol-5-yl)-2-hydroxy-propanamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-2-fluoro-N-(4-fluoro-1-methyl-indazol-5-yl)propanamide;
2-cyclopropyl-2-[(4S)-5-[5-[(4S)-2,2-dimethyltetrahydropyran-4-yl]-1-[(1 S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-N-(4-fluoro-1-methyl-indazol-2-ium-5-yl)-2-hydroxy-acetamide;
2-cyclopropyl-2-[(4S)-5-[5-[(4S)-2,2-dimethyltetrahydropyran-4-yl]-1-[(1 S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-N-(4-fluoro-1-methyl-indazol-2-ium-5-yl)-2-hydroxy-acetamide;
2-cyclopropyl-2-[(4S)-5-[5-[(4S)-2,2-dimethyltetrahydropyran-4-yl]-1-[(1 S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-2-fluoro-N-(4-fluoro-1-methyl-indazol-5-yl)acetamide;
2-cyclopropyl-2-[(4S)-5-[5-[(4S)-2,2-dimethyltetrahydropyran-4-yl]-1-[(1 S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-2-fluoro-N-(4-fluoro-1-methyl-indazol-5-yl)acetamide;
2-cyclopropyl-2-[(4S)-5-[5-[(4S)-2,2-dimethyltetrahydropyran-4-yl]-1-[(1 S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-2-fluoro-N-(1-methyl-6-isoquinolyl)acetamide;
2-cyclopropyl-2-[(4S)-5-[5-[(4S)-2,2-dimethyltetrahydropyran-4-yl]-1-[(1 S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-2-fluoro-N-(1-methyl-6-isoquinolyl)acetamide;
[1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-2-[(4-fluoro-1-methyl-indazol-2-ium-5-yl)amino]-1-methyl-2-oxo-ethyl]-dimethyl-ammonium;
[1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-2-[(4-fluoro-1-methyl-indazol-2-ium-5-yl)amino]-1-methyl-2-oxo-ethyl]-dimethyl-ammonium;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-N-(4-fluoro-1-methyl-indazol-5-yl)-2-methoxy-propanamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-N-(4-fluoro-1-methyl-indazol-5-yl)-2-methoxy-propanamide;
3-[(1S,2S)-1-[2-[(4S)-2-(1-cyclopropylpyrazol-4-yl)-3-[3-(4-fluoro-1-methyl-indazol-5-yl)-2-oxo-imidazol-1-yl]-4-methyl-spiro[4,6-dihydropyrazolo[4,3-c]pyridine-7,1′-cyclopropane]-5-carbonyl]-5-[(4S)-2,2-dimethyltetrahydropyran-4-yl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-3-[3-(4-fluoro-1-methyl-indazol-5-yl)-2-oxo-imidazol-1-yl]-4-methyl-2-[1-methyl-5-(trifluoromethyl)pyrazol-3-yl]-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-3-[3-(4-fluoro-1-methyl-indazol-5-yl)-2-oxo-imidazol-1-yl]-4-methyl-2-[1-methyl-5-(trifluoromethyl)pyrazol-3-yl]spiro[4,6-dihydropyrazolo[4,3-c]pyridine-7,1′-cyclopropane]-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-3-[3-(4-fluoro-1-methyl-indazol-5-yl)-2-oxo-imidazol-1-yl]-4-methyl-2-[5-(trifluoromethyl)-3-pyridyl]spiro[4,6-dihydropyrazolo[4,3-c]pyridine-7,1′-cyclopropane]-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-4-methyl-3-[5-(2-methyl-4-pyridyl)-1H-1,2,4-triazol-4-ium-3-yl]-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[3-(4-fluoro-1-methyl-indazol-5-yl)-2-oxo-3-azabicyclo[3.1.0]hexan-1-yl]-4-methyl-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[3-(4-fluoro-1-methyl-indazol-5-yl)-2-oxo-3-azabicyclo[3.1.0]hexan-1-yl]-4-methyl-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[5-(4-fluoro-1-methyl-indazol-2-ium-5-yl)-6-oxo-pyridazin-1-yl]-4-methyl-6,7-dihydro-4H-pyrazolo[4,3-c]pyridine-5-carbonyl]indol-1-yl]-2-methyl-cyclopropyl]-4H-1,2,4-oxadiazol-5-one;
1-[(4S)-5-[7-(2,2-dimethyltetrahydropyran-4-yl)-3-[1-(5-oxo-4H-1,2,4-oxadiazol-3-yl)cyclopropyl]indolizine-2-carbonyl]-2-(4-fluoro-3,5-dimethyl-phenyl)-4-methyl-6,7-dihydro-4H-pyrazolo[4,3-c]pyridin-3-yl]-N-(4-fluoro-1-methyl-indazol-5-yl)cyclopropanecarboxamide;
3-[1-[7-(2,2-dimethyltetrahydropyran-4-yl)-2-[(4S)-2-(4-fluoro-3,5-dimethyl-phenyl)-3-[1-(4-fluoro-1-methyl-indazol-5-yl)-2-oxo-pyrrolidin-3-yl]-4-methyl-6,7-dihydro-4H-pyrazolo[4,3-c]pyridine-5-carbonyl]indolizin-3-yl]cyclopropyl]-4H-1,2,4-oxadiazol-5-one;
3-[1-[7-(2,2-dimethyltetrahydropyran-4-yl)-2-[(4S)-2-(4-fluoro-3,5-dimethyl-phenyl)-3-[1-(4-fluoro-1-methyl-indazol-5-yl)-2-oxo-pyrrolidin-3-yl]-4-methyl-6,7-dihydro-4H-pyrazolo[4,3-c]pyridine-5-carbonyl]indolizin-3-yl]cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[5-(4-fluoro-1-methyl-indazol-5-yl)-6-oxo-pyrimidin-1-yl]-4-methyl-6,7-dihydro-4H-pyrazolo[4,3-c]pyridin-1-ium-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[5-(4-fluoro-1-methyl-indazol-5-yl)-6-oxo-pyrimidin-1-yl]-4-methyl-4,6,7,8-tetrahydropyrazolo[4,3-c]azepine-5-carbonyl]indol-1-yl]-2-methyl-cyclopropyl]-4H-1,2,4-oxadiazol-5-one;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-1-ium-3-yl]-4-(4-fluoro-1-methyl-indazol-5-yl)-2-methyl-morpholin-3-one;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-1-ium-3-yl]-4-(4-fluoro-1-methyl-indazol-5-yl)-2-methyl-morpholin-3-one;
3-[(1S,2S)-1-[5-[(4S)-2,2-dimethyltetrahydropyran-4-yl]-2-[(4S)-2-(4-fluoro-3,5-dimethyl-phenyl)-4-methyl-3-[4-(1-methylindazol-2-ium-5-yl)-5-oxo-1,2,4-triazol-1-yl]-4,6,7,8-tetrahydropyrazolo[4,3-c]azepine-5-carbonyl]indol-1-yl]-2-methyl-cyclopropyl]-4H-1,2,4-oxadiazol-5-one;
3-[(1S,2S)-1-[2-[(4S)-2-(3-cyclopropyl-4-fluoro-phenyl)-3-[4-(4-fluoro-1-methyl-indazol-2-ium-5-yl)-5-oxo-1,2,4-triazol-1-yl]-4-methyl-4,6,7,8-tetrahydropyrazolo[4,3-c]azepine-5-carbonyl]-5-[(4S)-2,2-dimethyltetrahydropyran-4-yl]indol-1-yl]-2-methyl-cyclopropyl]-4H-1,2,4-oxadiazol-5-one;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-N-(4-fluoro-1-methyl-indazol-2-ium-5-yl)-2-methyl-propanamide;
3-[(1S,2S)-1-[2-[(4S)-2-(4-fluoro-3,5-dimethyl-phenyl)-3-[3-(4-fluoro-1-methyl-indazol-5-yl)-2-oxo-imidazol-1-yl]-4-methyl-spiro[4,6-dihydropyrazolo[4,3-c]pyridine-7,3′-azetidine]-5-carbonyl]-5-tetrahydropyran-4-yl-indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-4-ethyl-2-(4-fluoro-3,5-dimethyl-phenyl)-3-[3-(4-fluoro-1-methyl-indazol-5-yl)-2-oxo-imidazol-1-yl]-4,6,7,8-tetrahydropyrazolo[4,3-c]azepin-1-ium-5-carbonyl]indol-1-yl]-2-methyl-cyclopropyl]-4H-1,2,4-oxadiazol-5-one;
3-[(1S,2S)-1-[5-(2,2-dimethyltetrahydropyran-4-yl)-2-[(4S)-2-(4-fluoro-3,5-dimethyl-phenyl)-3-[1-(4-fluoro-1-methyl-indazol-5-yl)-2-oxo-pyrrolidin-3-yl]-4-methyl-6,7-dihydro-4H-pyrazolo[4,3-c]pyridin-1-ium-5-carbonyl]indol-1-yl]-2-methyl-cyclopropyl]-4H-1,2,4-oxadiazol-5-one;
3-[(1S,2S)-1-[5-(2,2-dimethyltetrahydropyran-4-yl)-2-[(4S)-2-(4-fluoro-3,5-dimethyl-phenyl)-3-[1-(4-fluoro-1-methyl-indazol-5-yl)-2-oxo-pyrrolidin-3-yl]-4-methyl-6,7-dihydro-4H-pyrazolo[4,3-c]pyridin-1-ium-5-carbonyl]indol-1-yl]-2-methyl-cyclopropyl]-4H-1,2,4-oxadiazol-5-one;
3-[(1S,2S)-1-[2-[(4S)-3-[2-[2-(cyclopropoxy)-4-pyridyl]-3-oxo-pyridazin-4-yl]-2-(4-fluoro-3,5-dimethyl-phenyl)-4-methyl-6,7-dihydro-4H-pyrazolo[4,3-c]pyridine-5-carbonyl]-5-[(4S)-2,2-dimethyltetrahydropyran-4-yl]indol-1-yl]-2-methyl-cyclopropyl]-4H-1,2,4-oxadiazol-5-one;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-4-methyl-2-[5-(trifluoromethyl)-3-pyridyl]-6,7-dihydro-4H-pyrazolo[4,3-c]pyridin-3-yl]-N-(4-fluoro-1-methyl-indazol-5-yl)propanamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-4-methyl-2-[5-(trifluoromethyl)-3-pyridyl]-6,7-dihydro-4H-pyrazolo[4,3-c]pyridin-3-yl]-N-(4-fluoro-1-methyl-indazol-5-yl)propanamide;
N-(4-fluoro-1-methyl-indazol-5-yl)-2-[(4S)-4,7,7-trimethyl-5-[5-[(2S)-2-methylmorpholin-4-yl]-1-[(1S,2S)-2-methyl-1-(5-oxo-4H-1,2,4-oxadiazol-3-yl)cyclopropyl]indole-2-carbonyl]-2-[5-(trifluoromethyl)pyridin-1-ium-3-yl]-4,6-dihydropyrazolo[4,3-c]pyridin-3-yl]propanamide;
N-(4-fluoro-1-methyl-indazol-5-yl)-2-[(4S)-4,7,7-trimethyl-5-[5-[(2S)-2-methylmorpholin-4-yl]-1-[(1S,2S)-2-methyl-1-(5-oxo-4H-1,2,4-oxadiazol-3-yl)cyclopropyl]indole-2-carbonyl]-2-[5-(trifluoromethyl)pyridin-1-ium-3-yl]-4,6-dihydropyrazolo[4,3-c]pyridin-3-yl]propanamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-1′-(2,2,2-trifluoroethyl)spiro[4,6-dihydropyrazolo[4,3-c]pyridine-7,3′-azetidin-1-ium]-3-yl]-N-(4-fluoro-1-methyl-indazol-5-yl)cyclopropanecarboxamide;
3-[(1S,2S)-1-[2-[(4S)-2-(5-cyclopropylpyridin-1-ium-3-yl)-3-[3-(4-fluoro-1-methyl-indazol-5-yl)-2-oxo-imidazol-1-yl]-4-methyl-6,7-dihydro-4H-pyrazolo[4,3-c]pyridine-5-carbonyl]-5-[(4S)-2,2-dimethyltetrahydropyran-4-yl]indol-1-yl]-2-methyl-cyclopropyl]-4H-1,2,4-oxadiazol-5-one;
3-[(1S,2S)-1-[2-[(4S)-3-[1-[4-(cyclopropoxy)cyclohexyl]-6-oxo-pyrimidin-5-yl]-2-(4-fluoro-3,5-dimethyl-phenyl)-4-methyl-4,6,7,8-tetrahydropyrazolo[4,3-c]azepin-1-ium-5-carbonyl]-5-[(4S)-2,2-dimethyltetrahydropyran-4-yl]indol-1-yl]-2-methyl-cyclopropyl]-4H-1,2,4-oxadiazol-5-one;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4,7,7-trimethyl-6,8-dihydro-4H-pyrazolo[4,3-c]azepin-1-ium-3-yl]-N-(4-fluoro-1-methyl-indazol-5-yl)acetamide;
1-[(4S)-1′-(2,2-difluoroethyl)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-spiro[4,6-dihydropyrazolo[4,3-c]pyridine-7,3′-azetidin-1-ium]-3-yl]-N-(4-fluoro-1-methyl-indazol-5-yl)cyclopropanecarboxamide;
3-[(1S,2S)-1-[2-[(4S)-2-(4-fluoro-3,5-dimethyl-phenyl)-4-methyl-3-[2-(1-methylindazol-5-yl)-3-oxo-pyridazin-4-yl]spiro[4,6-dihydropyrazolo[4,3-c]pyridine-7,1′-cyclopropane]-5-carbonyl]-5-[(2S)-2-methylmorpholin-4-ium-4-yl]indol-1-yl]-2-methyl-cyclopropyl]-4H-1,2,4-oxadiazol-5-one;
3-[(1S,2S)-1-[2-[(4S)-3-[1-[2-(difluoromethyl)indazol-5-yl]-2-oxo-3-pyridyl]-2-(5-fluoro-4,6-dimethyl-pyridin-1-ium-2-yl)-4-methyl-6,7-dihydro-4H-pyrazolo[4,3-c]pyridine-5-carbonyl]-5-[(4S)-2,2-dimethyltetrahydropyran-4-yl]indol-1-yl]-2-methyl-cyclopropyl]-4H-1,2,4-oxadiazol-5-one;
3-[(1S,2S)-1-[2-[(4S)-2-(4-fluoro-3,5-dimethyl-phenyl)-3-[2-(4-fluoro-1-methyl-indazol-5-yl)-3-oxo-pyridazin-4-yl]-4-methyl-6,7-dihydro-4H-pyrazolo[4,3-c]pyridine-5-carbonyl]-5-[(2S)-2-methylmorpholin-4-ium-4-yl]indol-1-yl]-2-methyl-cyclopropyl]-4H-1,2,4-oxadiazol-5-one;
3-[(1S,2S)-1-[5-(2,2-difluoromorpholin-4-ium-4-yl)-2-[(4S)-2-(4-fluoro-3,5-dimethyl-phenyl)-3-[1-(4-fluoro-1-methyl-indazol-5-yl)-2-oxo-3-pyridyl]-4-methyl-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[2-[(4S)-2-(4-fluoro-3,5-dimethyl-phenyl)-3-[1-(4-fluoro-1-methyl-indazol-5-yl)-2-oxo-3-pyridyl]-4-methyl-6,7-dihydro-4H-pyrazolo[4,3-c]pyridine-5-carbonyl]-5-morpholin-4-ium-4-yl-indol-1-yl]-2-methyl-cyclopropyl]-4H-1,2,4-oxadiazol-5-one;
3-[(1S,2S)-1-[2-[(4S)-2-(4-fluoro-3,5-dimethyl-phenyl)-3-[1-(4-fluoro-1-methyl-indazol-5-yl)-6-oxo-pyrimidin-5-yl]-4-methyl-6,7-dihydro-4H-pyrazolo[4,3-c]pyridine-5-carbonyl]-5-[(2S)-2-methylmorpholin-4-ium-4-yl]indol-1-yl]-2-methyl-cyclopropyl]-4H-1,2,4-oxadiazol-5-one;
3-[(1S,2S)-1-[2-[(4S)-2-(4-fluoro-3,5-dimethyl-phenyl)-3-[1-(4-fluoro-1-methyl-indazol-5-yl)-6-oxo-pyrimidin-5-yl]-4-methyl-6,7-dihydro-4H-pyrazolo[4,3-c]pyridine-5-carbonyl]-5-morpholin-4-ium-4-yl-indol-1-yl]-2-methyl-cyclopropyl]-4H-1,2,4-oxadiazol-5-one;
3-[(1S,2S)-1-[2-[(4S)-2-(4-fluoro-3,5-dimethyl-phenyl)-3-[1-(4-fluoro-1-methyl-indazol-5-yl)-2-oxo-3-pyridyl]-4-methyl-6,7-dihydro-4H-pyrazolo[4,3-c]pyridine-5-carbonyl]-5-[(2S)-2-methylmorpholin-4-ium-4-yl]indol-1-yl]-2-methyl-cyclopropyl]-4H-1,2,4-oxadiazol-5-one;
3-[(1S,2S)-1-[2-[(4S)-2-(4-fluoro-3,5-dimethyl-phenyl)-4,7,7-trimethyl-3-[1-(1-methylindazol-5-yl)-2-oxo-3-pyridyl]-4,6-dihydropyrazolo[4,3-c]pyridine-5-carbonyl]-5-[(2S)-2-methylmorpholin-4-ium-4-yl]indol-1-yl]-2-methyl-cyclopropyl]-4H-1,2,4-oxadiazol-5-one;
3-[(1S,2S)-1-[2-[(4S)-3-[1-[2-(cyclopropoxy)-4-pyridyl]-2-oxo-3-pyridyl]-2-(5-fluoro-4,6-dimethyl-pyridin-1-ium-2-yl)-4-methyl-6,7-dihydro-4H-pyrazolo[4,3-c]pyridine-5-carbonyl]-5-[(4S)-2,2-dimethyltetrahydropyran-4-yl]indol-1-yl]-2-methyl-cyclopropyl]-4H-1,2,4-oxadiazol-5-one; and
3-[(1S,2S)-1-[2-[(4S)-2-(4-fluoro-3,5-dimethyl-phenyl)-3-[(1S,6S)-3-(4-fluoro-1-methyl-indazol-5-yl)-2-oxo-3-azabicyclo[4.1.0]heptan-1-yl]-4-methyl-6,7-dihydro-4H-pyrazolo[4,3-c]pyridine-5-carbonyl]-5-[(2S)-2-methylmorpholin-4-ium-4-yl]indol-1-yl]-2-methyl-cyclopropyl]-4H-1,2,4-oxadiazol-5-one,
or a pharmaceutically acceptable salt thereof.

112. A compound selected from:
2-[(4S)-2-(4-fluoro-3,5-dimethyl-phenyl)-4-methyl-5-[1-[(1S,2S)-2-methyl-1-(5-oxo-4H-1,2,4-oxadiazol-3-yl)cyclopropyl]-5-tetrahydropyran-4-yl-indole-2-carbonyl]-6,7-dihydro-4H-pyrazolo[4,3-c]pyridin-3-yl]-N-(4-fluoro-1-methyl-indazol-2-ium-5-yl)-2-methyl-propanamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-3-yl]-2-fluoro-N-(1-methyl-6-isoquinolyl)propanamide;
3-[(1S,2S)-1-[5-[(4S)-2,2-dimethyltetrahydropyran-4-yl]-2-[(4S)-2-(4-fluoro-3,5-dimethyl-phenyl)-3-[3-fluoro-1-(4-fluoro-1-methyl-indazol-2-ium-5-yl)-2-oxo-pyrrolidin-3-yl]-4-methyl-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[3-fluoro-1-(4-fluoro-1-methyl-indazol-2-ium-5-yl)-2-oxo-pyrrolidin-3-yl]-4-methyl-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[3-(4-fluoro-1-methyl-indazol-2-ium-5-yl)-2-oxo-3-azabicyclo[3.1.0]hexan-1-yl]-4-methyl-4,6,7,8-tetrahydropyrazolo[4,3-c]azepine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-4-methyl-3-[3-(2-methylquinolin-1-ium-6-yl)-2-oxo-3-azabicyclo[3.1.0]hexan-1-yl]-6,7-dihydro-4H-pyrazolo[4,3-c]pyridine-5-carbonyl]indol-1-yl]-2-methyl-cyclopropyl]-4H-1,2,4-oxadiazol-5-one;
2-[(4S)-2-(4-fluoro-3,5-dimethyl-phenyl)-4,7,7-trimethyl-5-[1-[(1S,2S)-2-methyl-1-(5-oxo-4H-1,2,4-oxadiazol-3-yl)cyclopropyl]-5-tetrahydropyran-4-yl-indole-2-carbonyl]-4,6-dihydropyrazolo[4,3-c]pyridin-3-yl]-N-(4-fluoro-1-methyl-indazol-5-yl)-2-methyl-propanamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4,7,7-trimethyl-4,6-dihydropyrazolo[4,3-c]pyridin-3-yl]-N-(4-fluoro-1-methyl-indazol-5-yl)-2-methyl-propanamide;
3-[(1R,2S)-1-[5-(2,2-dimethyltetrahydropyran-4-yl)-2-[(4S)-2-(4-fluoro-3,5-dimethyl-phenyl)-3-[3-(4-fluoro-1-methyl-indazol-5-yl)-2-oxo-imidazol-1-yl]-4-methyl-6,7-dihydro-4H-pyrazolo[4,3-c]pyridin-1-ium-5-carbonyl]phenyl]-2-methyl-cyclopropyl]-4H-1,2,4-oxadiazol-5-one;
2-[(4S)-2-(4-fluoro-3,5-dimethyl-phenyl)-4-methyl-5-[2-[(1S,2S)-2-methyl-1-(5-oxo-4H-1,2,4-oxadiazol-3-yl)cyclopropyl]-5-(1,3,7-trimethylindazol-6-yl)pyrazole-3-carbonyl]-6,7-dihydro-4H-pyrazolo[4,3-c]pyridin-3-yl]-N-(4-fluoro-1-methyl-indazol-5-yl)acetamide;
N-(1-cyclopropyl-4-fluoro-indazol-5-yl)-1-[(4S)-5-[5-(3,3-dimethyl-2-oxo-indolin-5-yl)-2-[(1S,2S)-2-methyl-1-(5-oxo-4H-1,2,4-oxadiazol-3-yl)cyclopropyl]pyrazole-3-carbonyl]-2-(4-fluoro-3,5-dimethyl-phenyl)-4-methyl-6,7-dihydro-4H-pyrazolo[4,3-c]pyridin-3-yl]cyclopropanecarboxamide;
N-(1-cyclopropyl-4-fluoro-indazol-5-yl)-1-[(4S)-2-(4-fluoro-3,5-dimethyl-phenyl)-4-methyl-5-[2-[(1S,2S)-2-methyl-1-(5-oxo-4H-1,2,4-oxadiazol-3-yl)cyclopropyl]-5-[1-methyl-3-(trifluoromethyl)pyrazol-5-yl]pyrazole-3-carbonyl]-6,7-dihydro-4H-pyrazolo[4,3-c]pyridin-3-yl]cyclopropanecarboxamide;
1-[(4S)-5-[5-(1,7-dimethylindazol-6-yl)-2-[(1S,2S)-2-methyl-1-(5-oxo-4H-1,2,4-oxadiazol-3-yl)cyclopropyl]pyrazole-3-carbonyl]-2-(4-fluoro-3,5-dimethyl-phenyl)-4-methyl-6,7-dihydro-4H-pyrazolo[4,3-c]pyridin-3-yl]-N-(4-fluoro-1-methyl-indazol-5-yl)cyclopropanecarboxamide;
N-(1-cyclopropyl-4-fluoro-indazol-5-yl)-1-[(4S)-2-(4-fluoro-3,5-dimethyl-phenyl)-4-methyl-5-[5-[4-(3-methyloxetan-3-yl)phenyl]-2-[(1S,2S)-2-methyl-1-(5-oxo-4H-1,2,4-oxadiazol-3-yl)cyclopropyl]pyrazole-3-carbonyl]-6,7-dihydro-4H-pyrazolo[4,3-c]pyridin-3-yl]cyclopropanecarboxamide;
1-[(4S)-5-[5-(1,7-dimethylindazol-6-yl)-2-[(1S,2S)-2-methyl-1-(5-oxo-4H-1,2,4-oxadiazol-3-yl)cyclopropyl]pyrazole-3-carbonyl]-2-(4-fluoro-3,5-dimethyl-phenyl)-4-methyl-spiro[4,6-dihydropyrazolo[4,3-c]pyridine-7,1′-cyclobutane]-3-yl]-N-(4-fluoro-1-methyl-indazol-5-yl)cyclopropanecarboxamide;
N-(1-cyclopropyl-4-fluoro-indazol-5-yl)-1-[(4S)-5-[5-(1,7-dimethylindazol-6-yl)-2-[(1S,2S)-2-methyl-1-(5-oxo-4H-1,2,4-oxadiazol-3-yl)cyclopropyl]pyrazole-3-carbonyl]-2-(4-fluoro-3,5-dimethyl-phenyl)-4-methyl-6,7-dihydro-4H-pyrazolo[4,3-c]pyridin-3-yl]cyclopropanecarboxamide;
N-(1-cyclopropyl-4-fluoro-indazol-5-yl)-1-[(4S)-2-(4-fluoro-3,5-dimethyl-phenyl)-5-[5-[4-(1-methoxycyclobutyl)phenyl]-2-[(1S,2S)-2-methyl-1-(5-oxo-4H-1,2,4-oxadiazol-3-yl)cyclopropyl]pyrazole-3-carbonyl]-4-methyl-6,7-dihydro-4H-pyrazolo[4,3-c]pyridin-3-yl]cyclopropanecarboxamide;
3-[(1S,2S)-1-[5-[(4S)-2-(4-fluoro-3,5-dimethyl-phenyl)-3-[3-(4-fluoro-1-methyl-indazol-5-yl)-2-oxo-imidazol-1-yl]-4-methyl-6,7-dihydro-4H-pyrazolo[4,3-c]pyridine-5-carbonyl]-3-(5-methyl-6-isoquinolyl)pyrazol-1-yl]-2-methyl-cyclopropyl]-4H-1,2,4-oxadiazol-5-one;
5-[4-[(4S)-2-(4-fluoro-3,5-dimethyl-phenyl)-3-[3-(4-fluoro-1-methyl-indazol-2-ium-5-yl)-2-oxo-imidazol-1-yl]-4-methyl-6,7-dihydro-4H-pyrazolo[4,3-c]pyridin-1-ium-5-carbonyl]-3-[(1R,2S)-1-(5-hydroxy-1,2,4-oxadiazole-2,4-diium-3-yl)-2-methyl-cyclopropyl]phenyl]-3,3-dimethyl-indolin-2-one;
N-(2,3-dimethyl-4-pyridyl)-2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-3-yl]acetamide;
N-(1-cyclopropylindazol-5-yl)-2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-spiro[6,8-dihydro-4H-pyrazolo[4,3-c]azepin-1-ium-7,1′-cyclopropane]-3-yl]acetamide;
N-(1-cyclopropylindazol-2-ium-5-yl)-2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4,7,7-trimethyl-6,8-dihydro-4H-pyrazolo[4,3-c]azepin-3-yl]acetamide;
N-(1-cyclopropylindazol-5-yl)-2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-spiro[4,6-dihydropyrazolo[4,3-c]pyridine-7,1′-cyclobutane]-3-yl]acetamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-spiro[6,8-dihydro-4H-pyrazolo[4,3-c]azepin-1-ium-7,1′-cyclopropane]-3-yl]-N-(4-fluoro-1-methyl-indazol-5-yl)acetamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-spiro[6,8-dihydro-4H-pyrazolo[4,3-c]azepine-7,1′-cyclopropane]-3-yl]-N-(1-methylisoquinolin-2-ium-6-yl)acetamide;
N-(1-cyclopropyl-4-fluoro-indazol-5-yl)-2-[(4S)-2-(4-fluoro-3,5-dimethyl-phenyl)-4-methyl-5-[1-[(1S,2S)-2-methyl-1-(5-oxo-4H-1,2,4-oxadiazol-3-yl)cyclopropyl]-5-tetrahydropyran-4-yl-indole-2-carbonyl]spiro[4,6-dihydropyrazolo[4,3-c]pyridine-7,1′-cyclobutane]-3-yl]acetamide;
N-(4,6-difluoro-1-methyl-indazol-5-yl)-2-[(4S)-2-(4-fluoro-3,5-dimethyl-phenyl)-4-methyl-5-[1-[(1S,2S)-2-methyl-1-(5-oxo-4H-1,2,4-oxadiazol-3-yl)cyclopropyl]-5-tetrahydropyran-4-yl-indole-2-carbonyl]spiro[4,6-dihydropyrazolo[4,3-c]pyridine-7,1′-cyclobutane]-3-yl]acetamide;
N-(1-cyclopropyl-4-fluoro-indazol-5-yl)-2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-spiro[4,6-dihydropyrazolo[4,3-c]pyridine-7,1′-cyclobutane]-3-yl]acetamide;
N-(4,6-difluoro-1-methyl-indazol-5-yl)-2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-spiro[6,8-dihydro-4H-pyrazolo[4,3-c]azepin-1-ium-7,1′-cyclopropane]-3-yl]acetamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-spiro[6,8-dihydro-4H-pyrazolo[4,3-c]azepine-7,1′-cyclopropane]-3-yl]-N-(3-methylisoquinolin-2-ium-6-yl)acetamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-spiro[6,8-dihydro-4H-pyrazolo[4,3-c]azepine-7,1′-cyclopropane]-3-yl]-N-(2-methylquinolin-1-ium-6-yl)acetamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4,7,7-trimethyl-6,8-dihydro-4H-pyrazolo[4,3-c]azepin-1-ium-3-yl]-N-(4-fluoro-1-methyl-indazol-5-yl)acetamide;
3-[(1S,2S)-1-[2-[(4S)-2-(4-fluoro-3,5-dimethyl-phenyl)-4-methyl-3-[3-(1-methylisoquinolin-2-ium-6-yl)-2-oxo-3-azabicyclo[4.1.0]heptan-1-yl]-6,7-dihydro-4H-pyrazolo[4,3-c]pyridine-5-carbonyl]-5-[(2S)-2-methylmorpholin-4-yl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[3-(7-fluoro-1-methyl-isoquinolin-2-ium-6-yl)-2-oxo-3-azabicyclo[4.1.0]heptan-1-yl]-4-methyl-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[3-(5-fluoro-1-methyl-isoquinolin-2-ium-6-yl)-2-oxo-3-azabicyclo[4.1.0]heptan-1-yl]-4-methyl-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[3-(7-fluoro-2-methyl-quinolin-1-ium-6-yl)-2-oxo-3-azabicyclo[4.1.0]heptan-1-yl]-4-methyl-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[2-[(4S)-3-[3-(1-cyclopropyl-4-fluoro-indazol-2-ium-5-yl)-2-oxo-3-azabicyclo[4.1.0]heptan-1-yl]-2-(4-fluoro-3,5-dimethyl-phenyl)-4-methyl-6,7-dihydro-4H-pyrazolo[4,3-c]pyridine-5-carbonyl]-5-[(4S)-2,2-dimethyltetrahydropyran-4-yl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[(1S,6S)-3-(4-hydroxycyclohexyl)-2-oxo-3-azabicyclo[4.1.0]heptan-1-yl]-4-methyl-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[5-[(4S)-2,2-dimethyltetrahydropyran-4-yl]-2-[(4S)-2-(4-fluoro-3,5-dimethyl-phenyl)-3-[3-(6-fluoro-1-methyl-indazol-2-ium-5-yl)-2-oxo-3-azabicyclo[4.1.0]heptan-1-yl]-4-methyl-6,7-dihydro-4H-pyrazolo[4,3-c]pyridine-5-carbonyl]indol-1-yl]-2-methyl-cyclopropyl]-4H-1,2,4-oxadiazol-5-one;
3-[(1S,2S)-1-[2-[(4S)-3-[(1S,6S)-3-[4-(cyclopropoxy)cyclohexyl]-2-oxo-3-azabicyclo[4.1.0]heptan-1-yl]-2-(4-fluoro-3,5-dimethyl-phenyl)-4-methyl-6,7-dihydro-4H-pyrazolo[4,3-c]pyridine-5-carbonyl]-5-[(4S)-2,2-dimethyltetrahydropyran-4-yl]indol-1-yl]-2-methyl-cyclopropyl]-4H-1,2,4-oxadiazol-5-one
N-(4-chloro-1-methyl-indazol-5-yl)-1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-1-ium-3-yl]cyclopropanecarboxamide;
1-[(4S)-2-(4-fluoro-3,5-dimethyl-phenyl)-4-methyl-5-[1-[(1S,2S)-2-methyl-1-(5-oxo-4H-1,2,4-oxadiazol-3-yl)cyclopropyl]-5-tetrahydropyran-4-yl-indole-2-carbonyl]spiro[4,6-dihydropyrazolo[4,3-c]pyridine-7,1′-cyclobutane]-3-yl]-N-(1-methylisoquinolin-2-ium-6-yl)cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-1-ium-3-yl]-N-(6-fluoro-1-methyl-indazol-5-yl)cyclopropanecarboxamide;
1-[(4S)-2-(4-fluoro-3,5-dimethyl-phenyl)-4-methyl-5-[1-[(1S,2S)-2-methyl-1-(5-oxo-4H-1,2,4-oxadiazol-3-yl)cyclopropyl]-5-tetrahydropyran-4-yl-indole-2-carbonyl]-6,7-dihydro-4H-pyrazolo[4,3-c]pyridin-1-ium-3-yl]-N-(6-fluoro-1-methyl-indazol-5-yl)cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-N-(5,6,7,8-tetrahydroisoquinolin-2-ium-7-yl)cyclopropanecarboxamide;
N-(4,6-difluoro-1-methyl-indazol-5-yl)-1-[(4S)-2-(4-fluoro-3,5-dimethyl-phenyl)-4-methyl-5-[1-[(1S,2S)-2-methyl-1-(5-oxo-4H-1,2,4-oxadiazol-3-yl)cyclopropyl]-5-tetrahydropyran-4-yl-indole-2-carbonyl]-6,7-dihydro-4H-pyrazolo[4,3-c]pyridin-1-ium-3-yl]cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-N-(5,6,7,8-tetrahydroisoquinolin-2-ium-7-yl)cyclopropanecarboxamide;
N-(1-cyclopropyl-4-fluoro-indazol-5-yl)-1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-1-ium-3-yl]cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-N-(2-methylquinolin-1-ium-6-yl)cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4,7,7-trimethyl-4,6-dihydropyrazolo[4,3-c]pyridin-1-ium-3-yl]-N-(1-methylindazol-5-yl)cyclopropanecarboxamide;
1-[(4S)-2-(4-fluoro-3,5-dimethyl-phenyl)-4-methyl-5-[1-[(1S,2S)-2-methyl-1-(5-oxo-4H-1,2,4-oxadiazol-3-yl)cyclopropyl]-5-tetrahydropyran-4-yl-indole-2-carbonyl]spiro[4,6-dihydropyrazolo[4,3-c]pyridin-1-ium-7,1′-cyclobutane]-3-yl]-N-(1-methylindazol-5-yl)cyclopropanecarboxamide;
N-(4,6-difluoro-1-methyl-indazol-5-yl)-1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-1-ium-3-yl]cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4,7,7-trimethyl-4,6-dihydropyrazolo[4,3-c]pyridin-1-ium-3-yl]-N-(4-fluoro-1-methyl-indazol-5-yl)cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-1-ium-3-yl]-N-(7-fluoro-2-methyl-indazol-5-yl)cyclopropanecarboxamide;
N-(4,6-difluoro-1-methyl-indazol-5-yl)-1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-1-ium-3-yl]cyclopropanecarboxamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-spiro[4,6-dihydropyrazolo[4,3-c]pyridin-1-ium-7,1′-cyclobutane]-3-yl]-N-(4-fluoro-1-methyl-indazol-5-yl)cyclopropanecarboxamide;
N-(1-cyclopropyl-4-fluoro-indazol-5-yl)-1-[(4S)-2-(4-fluoro-3,5-dimethyl-phenyl)-4-methyl-5-[2-[(1S,2S)-2-methyl-1-(5-oxo-4H-1,2,4-oxadiazol-3-yl)cyclopropyl]-5-(4-morpholinophenyl)pyrazole-3-carbonyl]-6,7-dihydro-4H-pyrazolo[4,3-c]pyridin-3-yl]cyclopropanecarboxamide;
N-(4-chloro-1-methyl-indazol-5-yl)-1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-4,6,7,8-tetrahydropyrazolo[4,3-c]azepin-1-ium-3-yl]cyclopropanecarboxamide;
1-[(4S)-2-(4-fluoro-3,5-dimethyl-phenyl)-4-methyl-5-[5-[(2S)-2-methylmorpholin-4-yl]-1-[(1S,2S)-2-methyl-1-(5-oxo-4H-1,2,4-oxadiazol-3-yl)cyclopropyl]indole-2-carbonyl]spiro[4,6-dihydropyrazolo[4,3-c]pyridine-7,1′-cyclobutane]-3-yl]-N-(1-methylisoquinolin-2-ium-6-yl)cyclopropanecarboxamide;
N-(1-cyclopropyl-4-fluoro-indazol-2-ium-5-yl)-2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4,7,7-trimethyl-4,6-dihydropyrazolo[4,3-c]pyridin-3-yl]-2-hydroxy-propanamide;
2-cyclopropyl-2-[(4S)-5-[5-[(4S)-2,2-dimethyltetrahydropyran-4-yl]-1-[(1 S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-2-fluoro-N-(1-methylindazol-5-yl)acetamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-1′-[(1-methylcyclopropyl)methyl]spiro[4,6-dihydropyrazolo[4,3-c]pyridine-7,3′-azetidin-1-ium]-3-yl]-N-(4-fluoro-1-methyl-indazol-5-yl)cyclopropanecarboxamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-spiro[4,6-dihydropyrazolo[4,3-c]pyridine-7,1′-cyclobutane]-3-yl]-N-(5-fluoro-1-methyl-6-isoquinolyl)acetamide;
N-(1-cyclopropyl-4-fluoro-indazol-5-yl)-1-[(4S)-5-[4-(3,3-dimethyl-2-oxo-indolin-5-yl)-2-[(1R,2S)-2-methyl-1-(5-oxo-4H-1,2,4-oxadiazol-3-yl)cyclopropyl]benzoyl]-2-(4-fluoro-3,5-dimethyl-phenyl)-4-methyl-6,7-dihydro-4H-pyrazolo[4,3-c]pyridin-3-yl]cyclopropanecarboxamide;
N-(1-cyclopropyl-4-fluoro-indazol-5-yl)-1-[(4S)-2-(4-fluoro-3,5-dimethyl-phenyl)-5-[4-isochroman-6-yl-2-[(1R,2S)-2-methyl-1-(5-oxo-4H-1,2,4-oxadiazol-3-yl)cyclopropyl]benzoyl]-4-methyl-6,7-dihydro-4H-pyrazolo[4,3-c]pyridin-3-yl]cyclopropanecarboxamide;
N-(1-cyclopropyl-4-fluoro-indazol-5-yl)-1-[(4S)-2-(4-fluoro-3,5-dimethyl-phenyl)-5-[4-(6-isoquinolyl)-2-[(1R,2S)-2-methyl-1-(5-oxo-4H-1,2,4-oxadiazol-3-yl)cyclopropyl]benzoyl]-4-methyl-6,7-dihydro-4H-pyrazolo[4,3-c]pyridin-3-yl]cyclopropanecarboxamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-spiro[4,6-dihydropyrazolo[4,3-c]pyridine-7,1′-cyclobutane]-3-yl]-N-(7-fluoro-1-methyl-6-isoquinolyl)acetamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4,7,7-trimethyl-4,6-dihydropyrazolo[4,3-c]pyridin-3-yl]-N-(5-fluoro-2-methyl-6-quinolyl)acetamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4,7,7-trimethyl-4,6-dihydropyrazolo[4,3-c]pyridin-3-yl]-N-(5-fluoro-1-methyl-6-isoquinolyl)acetamide;
2-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-spiro[4,6-dihydropyrazolo[4,3-c]pyridine-7,1′-cyclobutane]-3-yl]-N-(5-fluoro-2-methyl-6-quinolyl)acetamide;
1-[(4S)-5-[5-[(4S)-2,2-dimethyltetrahydropyran-4-yl]-1-[(1S,2S)-2-methyl-1-(5-oxo-4H-1,2,4-oxadiazol-3-yl)cyclopropyl]indole-2-carbonyl]-2-(4-fluoro-3,5-dimethyl-phenyl)-4-methyl-6,7-dihydro-4H-pyrazolo[4,3-c]pyridin-3-yl]-N-(5-fluoro-2-methyl-quinolin-1-ium-6-yl)cyclopropanecarboxamide; and
3-[(1S,2S)-1-[5-[(4S)-2,2-dimethyltetrahydropyran-4-yl]-2-[(4S)-2-(4-fluoro-3,5-dimethyl-phenyl)-3-[3-(5-fluoro-2-methyl-quinolin-1-ium-6-yl)-2-oxo-3-azabicyclo[4.1.0]heptan-1-yl]-4-methyl-6,7-dihydro-4H-pyrazolo[4,3-c]pyridine-5-carbonyl]indol-1-yl]-2-methyl-cyclopropyl]-4H-1,2,4-oxadiazol-5-one,
or a pharmaceutically acceptable salt thereof.

113. A pharmaceutical composition which comprises a compound according to any one of claims 1 to 112, or a pharmaceutically acceptable salt thereof, and a pharmaceutically acceptable carrier.

114. A method for treating obesity or non-insulin-dependent diabetes mellitus (Type 2 diabetes) which comprises administering to a subject in need of such treatment (i) a therapeutically effective amount of a compound according to any one of claims 1 to 112, or a pharmaceutically acceptable salt thereof, or (ii) a pharmaceutical composition according to claim 113.

115. Use of a compound according to any one of claims 1 to 112, or a pharmaceutically acceptable salt thereof, in the manufacture of a medicament for treating obesity or non-insulin-dependent diabetes mellitus (Type 2 diabetes).
